(19) United States
(12) Patent Application Publication
(10) Pub. No.: US 2004/0033495 A1
(43) Pub. Date: Feb. 19, 2004

(54) Methods of diagnosis of angiogenesis, compositions and methods of screening for angiogenesis modulators

(75) Inventors: Richard Murray, Cupertino, CA (US); Richard Glynne, Palo Alto, CA (US); Susan R. Watson, El Cerrito, CA (US); Natasha Aziz, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Eos Biotechnology, Inc., South San Francisco, CA (US)

(21) Appl. No.: 10/211,462

(22) Filed: Aug. 1, 2002

Related U.S. Application Data
(60) Provisional application No. 60/310,025, filed on Aug. 3, 2001.
(60) Provisional application No. 60/334,244, filed on Nov. 29, 2001.

(51) Int. Cl. C12Q 1/68; G01N 33/574; C07H 21/04; C12P 21/02; C12N 5/06

(52) U.S. Cl. 435/6; 435/7.23; 435/69.1; 435/320.1; 435/325; 536/23.2

(57) ABSTRACT

Described herein are methods and compositions that can be used for diagnosis and treatment of angiogenic phenotypes and angiogenesis-associated diseases. Also described herein are methods that can be used to identify modulators of angiogenesis.

### CROSS-REFERENCES TO RELATED APPLICATIONS

This application claims priority to U.S. Ser. No 09/784,356, filed Feb. 14, 2001; U.S. Ser. No. 09/791,390, filed Feb. 22, 2001; U.S. Ser. No. 60/310,025, filed Aug. 3, 2001, and U.S. Ser. No. 60/334,244, filed Nov. 29, 2001, each of which is herein incorporated by reference in its entirety.

### FIELD OF THE INVENTION

The invention relates to the identification of nucleic acid and protein expression profiles and nucleic acids, products, and antibodies thereto that are involved in angiogenesis; and to the use of such expression profiles and compositions in diagnosis and therapy of angiogenesis. The invention further relates to methods for identifying and using agents and/or targets that modulate angiogenesis.

### BACKGROUND OF THE INVENTION

Both vasculogenesis, the development of an interactive vascular system comprising arteries and veins, and angiogenesis, the generation of new blood vessels, play a role in embryonic development. In contrast, angiogenesis is limited in a normal adult to the placenta, ovary, endometrium and sites of wound healing. However, angiogenesis, or its absence, plays an important role in the maintenance of a variety of pathological states. Some of these states are characterized by neovascularization, e.g., cancer, diabetic retinopathy, glaucoma, and age related macular degeneration. Others, e.g., stroke, infertility, heart disease, ulcers, and scleroderma, are diseases of angiogenic insufficiency.

Angiogenesis has a number of stages (see, e.g., Folkman, J.Natl Cancer Inst. 82:4-6, 1990; Firestein, J Clin Invest. 103:3-4, 1999; Koch, Arthritis Rheum.41:951-62, 1998; Carter, Oncologist 5(Suppl 1):51-4, 2000; Browder et al., Cancer Res. 60:1878-86, 2000; and Zhu and Witte, Invest New Drugs 17:195-212, 1999). The early stages of angiogenesis include endothelial cell protease production, migration of cells, and proliferation. The early stages also appear to require some growth factors, with VEGF, TGF-α, angiostatin, and selected chemokines all putatively playing a role. Later stages of angiogenesis include population of the vessels with mural cells (pericytes or smooth muscle cells), basement membrane production, and the induction of vessel bed specializations. The final stages of vessel formation include what is known as “remodeling”, wherein a forming vasculature becomes a stable, mature vessel bed. Thus, the process is highly dynamic, often requiring coordinated spatial and temporal waves of gene expression.

Conversely, the complex process may be subject to disruption by interfering with one or more critical steps. Thus, the lack of understanding of the dynamics of angiogenesis prevents therapeutic intervention in serious diseases such as those indicated. It is an object of the invention to provide methods that can be used to screen compounds for the ability to modulate angiogenesis. Additionally, it is an object to provide molecular targets for therapeutic intervention in disease states which either have an undesirable excess or a deficit in angiogenesis. The present invention provides solutions to both.

### SUMMARY OF THE INVENTION

The present invention provides compositions and methods for detecting or modulating angiogenesis associated sequences.

In one aspect, the invention provides a method of detecting an angiogenesis-associated transcript in a cell in a patient, the method comprising contacting a biological sample from the patient with a polynucleotide that selectively hybridized to a sequence at least 80% identical to a sequence as shown in Tables 1-8. In one embodiment, the biological sample is a tissue sample. In another embodiment, the biological sample comprises isolated nucleic acids, which are often mRNA.

In another embodiment, the method further comprises the step of amplifying nucleic acids before the step of contacting the biological sample with the polynucleotide. Often, the polynucleotide comprises a sequence as shown in Tables 1-8. The polynucleotide can be labeled, for example, with a fluorescent label and can be immobilized on a solid surface.

In other embodiments the patient is undergoing a therapeutic regimen to treat a disease associated with angiogenesis or the patient is suspected of having an angiogenesis-associated disorder.

In another aspect, the invention comprises an isolated nucleic acid molecule consisting of a polynucleotide sequence as shown in Tables 1-8. The nucleic acid molecule can be labeled, for example, with a fluorescent label,

In other aspects, the invention provides an expression vector comprising an isolated nucleic acid molecule consisting of a polynucleotide sequence as shown in Tables 1-8 or a host cell comprising the expression vector.

In another embodiment, the isolated nucleic acid molecule encodes a polypeptide having an amino acid sequence as shown in Table 8.

In another aspect, the invention provides an isolated polypeptide which is encoded by a nucleic acid molecule having polynucleotide sequence as shown in Tables 1-8. In one embodiment, the isolated polypeptide has an amino acid sequence as shown in Table 8.

In another embodiment, the invention provides an antibody that specifically binds a polypeptide that has an amino acid sequence as shown in Table 8 or which is encoded by a nucleotide sequence of Tables 1-8. The antibody can be conjugated or fused to an effector component such as a fluorescent label, a toxin, or a radioisotope. In some embodiments, the antibody is an antibody fragment or a humanized antibody.

In another aspect, the invention provides a method of detecting a cell undergoing angiogenesis in a biological sample from a patient, the method comprising contacting the biological sample with an antibody that specifically binds to a polypeptide that has an amino acid sequence as shown in Table 8 or which is encoded by a nucleotide sequence of Tables 1-8. In some embodiments, the antibody is further conjugated or fused to an effector component, for example, a fluorescent label.

In another embodiment, the invention provides a method of detecting antibodies specific to angiogenesis in a patient, the method comprising contacting a biological sample from the patient with a polypeptide which is encoded by a nucleotide sequence of Tables 1-8.

The invention also provides a method of identifying a compound that modulates the activity of an angiogenesis-associated polypeptide, the method comprising the steps of: (i) contacting the compound with a polypeptide that comprises at least 80% identity to an amino acid sequence as shown in Table 8 or which is encoded by a nucleotide sequence of Tables 1-8; and (ii) detecting an increase or a decrease in the activity of the polypeptide. In one embodiment, the polypeptide has an amino acid sequence as shown in Table 8 or is a polypeptide encoded by a nucleotide sequence of Tables 1-8. In another embodiment, the polypeptide is expressed in a cell.

The invention also provides a method of identifying a compound that modulates angiogenesis, the method comprising steps of: (i) contacting the compound with a cell undergoing angiogenesis; and (ii) detecting an increase or a decrease in the expression of a polypeptide sequence as shown in Table 8 or a polypeptide which is encoded by a nucleotide sequence of Tables 1-8. In one embodiment, the detecting step comprises hybridizing a nucleic acid sample from the cell with a polynucleotide that selectively hybridizes to a sequence at least 80% identical to a sequence as shown in Tables 1-8. In another embodiment, the method further comprises detecting an increase or decrease in the expression of a second sequence as shown in Table 8 or a polypeptide which is encoded by a nucleotide sequence of Tables 1-8.

In another embodiment, the invention provides a method of inhibiting angiogenesis in a cell that expresses a polypeptide at least 80% identical to a sequence as shown in Table 8 or which is 80% identical to a polypeptide encoded by a nucleotide sequence of Tables 1-8, the method comprising the step of contacting the cell with a therapeutically effective amount of an inhibitor of the polypeptide. In one embodiment, the polypeptide has an amino acid sequence shown in Table 8 or is a polypeptide which is encoded by a nucleotide sequence of Tables 1-8. In another embodiment, the inhibitor is an antibody.

In other embodiments, the invention provides a method of activating angiogenesis in a cell that expresses a polypeptide at least 80% identical to a sequence as shown in Table 8 or at least 80% identical to a polypeptide which is encoded by a nucleotide sequence of Tables 1-8, the method comprising the step of contacting the cell with a therapeutically effective amount of an activator of the polypeptide. In one embodiment, the polypeptide has an amino acid sequence shown in Table 8 or is a polypeptide which is encoded by a nucleotide sequence of Tables 1-8.

Other aspects of the invention will become apparent to the skilled artisan by the following description of the invention.

Tables 1-8 provide nucleotide sequence of genes that exhibit changes in expression levels as a function of time in tissue undergoing angiogenesis compared to tissue that is not.

### DESCRIPTION OF THE SPECIFIC EMBODIMENTS

In accordance with the objects outlined above, the present invention provides novel methods for diagnosis and treatment of disorders associated with angiogenesis (sometimes referred to herein as angiogenesis disorders or AD), as well as methods for screening for compositions which modulate angiogenesis. By “disorder associated with angiogenesis” or “disease associated with angiogenesis” herein is meant a disease state which is marked by either an excess or a deficit of blood vessel development. Angiogenesis disorders asociated with increased angiogenesis include, but are not limited to, cancer and proliferative diabetic retinopathy. Pathological states for which it may be desirable to increase angiogenesis include stroke, heart disease, infertility, ulcers, wound healing, ischemia, and scleradoma. Solid tumors typically require angiogenesis to support or sustain growth, e.g., breast, colon, lung, brain, bladder, and prostate tumors. Other AD include, e.g., arthritis, inflammatory bowel disease, diabetis retinopathy, macular degeneration, atherosclerosis, and psoriasis. Also provided are methods for treating AD.

Definitions

The term “angiogenesis protein” or “angiogenesis polynucleotide” refers to nucleic acid and polypeptide polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 96%, 97%, 98% or 99% or greater amino acid sequence identity, preferably over a region of over a region of at least about 25, 50, 100, 200, 500, 1000, or more amino acids, to an angiogenesis protein sequence of Table 8; (2) bind to antibodies, e.g., polyclonal antibodies, raised against an immunogen comprising an amino acid sequence of Table 8, and conservatively modified variants thereof; (3) specifically hybridize under stringent hybridization conditions to an anti-sense strand corresponding to a nucleic acid sequence of Tables 1-8 and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a sense sequence corresponding to one set out in Tables 1-8. A polynucleotide or polypeptide sequence is typically from a mammal including, but not limited to, primate, e.g., human; rodent, e.g., rat, mouse, hamster; cow, pig, horse, sheep, or any mammal. An “angiogenesis polypeptide” and an “angiogenesis polynucleotide,” include both naturally occurring or recombinant.

A “full length” angiogenesis protein or nucleic acid refers to an agiogenesis polypeptide or polynucleotide sequence, or a variant thereof, that contains all of the elements normally contained in one or more naturally occurring, wild type angiogenesis polynucleotide or polypeptide sequences. The “full length” may be prior to, or after, various stages of post-translation processing.

“Biological sample” as used herein is a sample of biological tissue or fluid that contains nucleic acids or polypeptides, e.g., of an angiogenic protein. Such samples include, but are not limited to, tissue isolated from primates, e.g., humans, or rodents, e.g., mice, and rats. Biological samples may also include sections of tissues such as biopsy and autopsy samples, and frozen sections taken for histologic purposes. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

“Providing a biological sample” means to obtain a biological sample for use in methods described in this invention. Most often, this will be done by removing a sample of cells from an animal, but can also be accomplished by using previously isolated cells (e.g., isolated by another person, at another time, and/or for another purpose), or by performing the methods of the invention in vivo. Archival tissues, having treatment or outcome histroy, will be particularly useful.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, 91%, 92%, 93%, 94%, 95% 96%, 97%, 98%, 99%, or higher identity over a specified region (e.g., SEQ ID NOS:1-229), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

A “host cell” is a naturally occurring cell or a transformed cell that contains an expression vector and supports the replication or expression of the expression vector. Host cells may be cultured cells, explants, cells in vivo, and the like. Host cells may be prokaryotic cells such as E. coli, or eukaryotic cells such as yeast, insect, amphibian, or mammalian cells such as CHO, HeLa, and the like (see, e.g., the American Type Culture Collection catalog or web site, www.atcc.org).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 25 to approximately 500 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed, usually by the noncovalent association of independent tertiary units. Anisotropic terms are also known as energy terms.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

An “effector” or “effector moiety” or “effector component” is a molecule that is bound (or linked, or conjugated), either covalently, through a linker or a chemical bond, or noncovalently, through ionic, van der Waals, electrostatic, or hydrogen bonds, to an antibody. The “effector” can be a variety of molecules including, for example, detection moieties including radioactive compounds, fluroescent compounds, an enzyme or substrate, tags such as epitope tags, a toxin; a chemotherapeutic agent; a lipase; an antibiotic; or a radioisotope emitting “hard” e.g., beta radiation.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. Alternatively, method using high affinity interactions may achieve the same results where one of a pair of binding partners binds to the other, e.g., biotin, streptavidin.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al

The phrase “functional effects” in the context of assays for testing compounds that modulate activity of an angiogenesis protein includes the determination of a parameter that is indirectly or directly under the influence of the angiogenesis protein, e.g., a functional, physical, or chemical effect, such as the ability to increase or decrease angiogenesis. It includes binding activity, the ability of cells to proliferate, expression in cells undergoing angiogenesis, and other characteristics of angiogenic cells. “Functional effects” include in vitro, in vivo, and ex vivo activities.

By “determining the functional effect” is meant assaying for a compound that increases or decreases a parameter that is indirectly or directly under the influence of an angiogenesis protein sequence, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties for the protein, measuring inducible markers or transcriptional activation of the angiogenesis protein; measuring binding activity or binding assays, e.g. binding to antibodies, and measuring cellular proliferation, particularly endothelial cell proliferation, cell viability, cell division especially of endothelial cells, lumen formation and capillary or vessel growth or formation. Determination of the functional effect of a compound on angiogenesis can also be performed using angiogenesis assays known to those of skill in the art such as an in vitro assays, e.g., in vitro endothelial cell tube formation assays, and other assays such as the chick CAM assay, the mouse corneal assay, and assays that assess vascularization of an implanted tumor. The functional effects can be evaluated by many means known to those skilled in the art, e.g., microscopy for quantitative or qualitative measures of alterations in morphological features, e.g., tube or blood vessel formation, measurement of changes in RNA or protein levels for angiogenesis-associated sequences, measurement of RNA stability, identification of downstream or reporter gene expression (CAT, luciferase, β-gal, GFP and the like), e.g., via chemiluminescence, fluorescence, colorimetric reactions, antibody binding, inducible markers, and ligand binding assays.

“Inhibitors”, “activators”, and “modulators” of angiogenic polynucleotide and polypeptide sequences are used to refer to activating, inhibitory, or modulating molecules identified using in vitro and in vivo assays of angiogenic polynucleotide and polypeptide sequences. Inhibitors are compounds that, e.g., bind to, partially or totally block activity, decrease, prevent, delay activation, inactivate, desensitize, or down regulate the activity or expression of angiogenesis proteins, e.g., antagonists. “Activators” are compounds that increase, open, activate, facilitate, enhance activation, sensitize, agonize, or up regulate angiogenesis protein activity. Inhibitors, activators, or modulators also include genetically modified versions of angiogenesis proteins, e.g., versions with altered activity, as well as naturally occurring and synthetic ligands, antagonists, agonists, antibodies, small chemical molecules and the like. Such assays for inhibitors and activators include, e.g., expressing the angiogenic protein in vitro, in cells, or cell membranes, applying putative modulator compounds, and then determining the functional effects on activity, as described above. Activators and inhibitors of angiogenesis can also be identified by incubating angiogenic cells with the test compound and determining increases or decreases in the expression of 1 or more angiogenesis proteins, e.g., 1, 2, 3, 4, 5, 10, 15, 20, 25, 30, 40, 50 or more angiogenesis proteins, such as angiogenesis proteins comprising the sequences set out in Table 8.

Samples or assays comprising angiogenesis proteins that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of a polypeptide is achieved when the activity value relative to the control is about 80%, preferably 50%, more preferably 25-0%. Activation of an angiogenesis polypeptide is achieved when the activity value relative to the control (untreated with activators) is 110%, more preferably 150%, more preferably 200-500% (i.e., two to five fold higher relative to the control), more preferably 1000-3000% higher.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The detailed description of the invention includes discussion of the following aspects of the invention:

Expression of angiogenesis-associated sequences

Informatics

Angiogenesis-associated sequences

Detection of angiogenesis sequence for diagnostic and therapeutic applications

Modulators of angiogenesis

Methods of identifying variant angiogenesis-associated sequences

Administration of pharmaceutical and vaccine compositions

Kits for use in diagnostic and/or prognostic applications.

Expression of Angiogenesis-associated Sequences

In one aspect, the expression levels of genes are determined in different patient samples for which diagnosis information is desired, to provide expression profiles. An expression profile of a particular sample is essentially a “fingerprint” of the state of the sample; while two states may have any particular gene similarly expressed, the evaluation of a number of genes simultaneously allows the generation of a gene expression profile that is unique to the state of the cell. That is, normal tissue may be distinguished from AD tissue. By comparing expression profiles of tissue in known different angiogenesis states, information regarding which genes are important (including both up- and down-regulation of genes) in each of these states is obtained. The identification of sequences that are differentially expressed in angiogenic versus non-angiogenic tissue allows the use of this information in a number of ways. For example, a particular treatment regime may be evaluated: does a chemotherapeutic drug act to down-regulate angiogenesis, and thus tumor growth or recurrence, in a particular patient. Similarly, diagnosis and treatment outcomes may be done or confirmed by comparing patient samples with the known expression profiles. Angiogenic tissue can also be analyzed to determine the stage of angiogenesis in the tissue. Furthermore, these gene expression profiles (or individual genes) allow screening of drug candidates with an eye to mimicking or altering a particular expression profile; for example, screening can be done for drugs that suppress the angiogenic expression profile. This may be done by making biochips comprising sets of the important angiogenesis genes, which can then be used in these screens. These methods can also be done on the protein basis; that is, protein expression levels of the angiogenic proteins can be evaluated for diagnostic purposes or to screen candidate agents. In addition, the angiogenic nucleic acid sequences can be administered for gene therapy purposes, including the administration of antisense nucleic acids, or the angiogenic proteins (including antibodies and other modulators thereof) administered as therapeutic drugs.

Thus the present invention provides nucleic acid and protein sequences that are differentially expressed in angiogenesis, herein termed “angiogenesis sequences”. As outlined below, angiogenesis sequences include those that are up-regulated (i.e. expressed at a higher level) in disorders associated with angiogenesis, as well as those that are down-regulated (i.e. expressed at a lower level). In a preferred embodiment, the angiogenesis sequences are from humans; however, as will be appreciated by those in the art, angiogenesis sequences from other organisms may be useful in animal models of disease and drug evaluation; thus, other angiogenesis sequences are provided, from vertebrates, including mammals, including rodents (rats, mice, hamsters, guinea pigs, etc.), primates, farm animals (including sheep, goats, pigs, cows, horses, etc). Angiogenesis sequences from other organisms may be obtained using the techniques outlined below.

Angiogenesis sequences can include both nucleic acid and amino acid sequences. In a preferred embodiment, the angiogenesis sequences are recombinant nucleic acids. By the term “recombinant nucleic acid” herein is meant nucleic acid, originally formed in vitro, in general, by the manipulation of nucleic acid e.g., using polymerases and endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of an angiogenesis protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below.

In a preferred embodiment, the angiogenesis sequences are nucleic acids. As will be appreciated by those in the art and is more fully outlined below, angiogenesis sequences are useful in a variety of applications, including diagnostic applications, which will detect naturally occurring nucleic acids, as well as screening applications; for example, biochips comprising nucleic acid probes to the angiogenesis sequences can be generated. In the broadest sense, then, by “nucleic acid” or “oligonucleotide” or grammatical equivalents herein means at least two nucleotides covalently linked together. A nucleic acid of the present invention will generally contain phosphodiester bonds, although in some cases, nucleic acid analogs are included that may have alternate backbones, comprising, for example, phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids. Modifications of the ribose-phosphate backbone may be done for a variety of reasons, for example to increase the stability and half-life of such molecules in physiological environments or as probes on a biochip.

As will be appreciated by those in the art, nucleic acid analogs may find use in the present invention. In addition, mixtures of naturally occurring nucleic acids and analogs can be made; alternatively, mixtures of different nucleic acid analogs, and mixtures of naturally occurring nucleic acids and analogs may be made.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched basepairs. DNA and RNA typically exhibit a 2-4° C. drop in Tₘ for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

The nucleic acids may be single stranded or double stranded, as specified, or contain portions of both double stranded or single stranded sequence. As will be appreciated by those in the art, the depiction of a single strand also defines the sequence of the complementary strand; thus the sequences described herein also provide the complement of the sequence. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

An angiogenesis sequence can be initially identified by substantial nucleic acid and/or amino acid sequence homology to the angiogenesis sequences outlined herein. Such homology can be based upon the overall nucleic acid or amino acid sequence, and is generally determined as outlined below, using either homology programs or hybridization conditions.

For identifying angiogenesis-associated sequences, the angiogenesis screen typically includes comparing genes identified in a modification of an in vitro model of angiogenesis as described in Hiraoka, Cell 95:365 (1998) with genes identified in controls. Samples of normal tissue and tissue undergoing angiogenesis are applied to biochips comprising nucleic acid probes. The samples are first microdissected, if applicable, and treated as is known in the art for the preparation of mRNA. Suitable biochips are commercially available, for example from Affymetrix. Gene expression profiles as described herein are generated and the data analyzed.

In a preferred embodiment, the genes showing changes in expression as between normal and disease states are compared to genes expressed in other normal tissues, including, but not limited to lung, heart, brain, liver, breast, kidney, muscle, prostate, small intestine, large intestine, spleen, bone and placenta. In a preferred embodiment, those genes identified during the angiogenesis screen that are expressed in any significant amount in other tissues are removed from the profile, although in some embodiments, this is not necessary. That is, when screening for drugs, it is usually preferable that the target be disease specific, to minimize possible side effects.

In a preferred embodiment, angiogenesis sequences are those that are up-regulated in angiogenesis disorders; that is, the expression of these genes is higher in the disease tissue as compared to normal tissue. “Up-regulation” as used herein means at least about a two-fold change, preferably at least about a three fold change, with at least about five-fold or higher being preferred. All accession numbers herein are for the GenBank sequence database and the sequences of the accession numbers are hereby expressly incorporated by reference. GenBank is known in the art, see, e.g., Benson, D A, et al., Nucleic Acids Research 26:1-7 (1998) and http://www.ncbi.nlm.nih.gov/. Sequences are also avialable in other databases, e.g., European Molecular Biology Laboratory (EMBL) and DNA Database of Japan (DDBJ). In addition, most preferred genes were found to be expressed in a limited amount or not at all in heart, brain, lung, liver, breast, kidney, prostate, small intestine and spleen.

In another preferred embodiment, angiogenesis sequences are those that are down-regulated in the angiogenesis disorder; that is, the expression of these genes is lower in angiogenic tissue as compared to normal tissue. “Down-regulation” as used herein means at least about a two-fold change, preferably at least about a three fold change, with at least about five-fold or higher being preferred.

Angiogenesis sequences according to the invention may be classified into discrete clusters of sequences based on common expression profiles of the sequences. Expression levels of angiogenesis sequences may increase or decrease as a function of time in a manner that correlates with the induction of angiogenesis. Alternatively, expression levels of angiogenesis sequences may both increase and decrease as a function of time. For example, expression levels of some angiogenesis sequences are temporarily induced or diminished during the switch to the angiogenesis phenotype, followed by a return to baseline expression levels. Tables 1-8 provides genes, the mRNA expression of which varies as a function of time in angiogenesis tissue when compared to normal tissue.

In a particularly preferred embodiment, angiogenesis sequences are those that are induced for a period of time, typically by positive angiogenic factors, followed by a return to the baseline levels. Sequences that are temporarily induced provide a means to target angiogenesis tissue, for example neovascularized tumors, at a particular stage of angiogenesis, while avoiding rapidly growing tissue that require perpetual vascularization. Such positive angiogenic factors include αFGF, βFGF, VEGF, angiogenin and the like.

Induced angiogenesis sequences also are further categorized with respect to the timing of induction. For example, some angiogenesis genes may be induced at an early time period, such as within 10 minutes of the induction of angiogenesis. Others may be induced later, such as between 5 and 60 minutes, while yet others may be induced for a time period of about two hours or more followed by a return to baseline expression levels.

In another preferred embodiment are angiogenesis sequences that are inhibited or reduced as a function of time followed by a return to “normal” expression levels. Inhibitors of angiogenesis are examples of molecules that have this expression profile. These sequences also can be further divided into groups depending on the timing of diminished expression. For example, some molecules may display reduced expression within 10 minutes of the induction of angiogenesis. Others may be diminished later, such as between 5 and 60 minutes, while others may be diminished for a time period of about two hours or more followed by a return to baseline. Examples of such negative angiogenic factors include thrombospondin and endostatin to name a few.

In yet another preferred embodiment are angiogenesis sequences that are induced for prolonged periods. These sequences are typically associated with induction of angiogenesis and may participate in induction and/or maintenance of the angiogenesis phenotype.

In another preferred embodiment are angiogenesis sequences, the expression of which is reduced or diminished for prolonged periods in angiogenic tissue. These sequences are typically angiogenesis inhibitors and their diminution is correlated with an increase in angiogenesis.

Informatics

The ability to identify genes that undergo changes in expression with time during angiogenesis can additionally provide high-resolution, high-sensitivity datasets which can be used in the areas of diagnostics, therapeutics, drug development, biosensor development, and other related areas. For example, the expression profiles can be used in diagnostic or prognostic evaluation of patients with angiogenesis-associated disease. Or as another example, subcellular toxicological information can be generated to better direct drug structure and activity correlation (see, Anderson, L., “Pharmaceutical Proteomics: Targets, Mechanism, and Function,” paper presented at the IBC Proteomics conference, Coronado, Calif. (Jun. 11-12, 1998)). Subcellular toxicological information can also be utilized in a biological sensor device to predict the likely toxicological effect of chemical exposures and likely tolerable exposure thresholds (see, U.S. Pat. No. 5,811,231). Similar advantages accrue from datasets relevant to other biomolecules and bioactive agents (e.g., nucleic acids, saccharides, lipids, drugs, and the like).

Thus, in another embodiment, the present invention provides a database that includes at least one set of data assay data. The data contained in the database is acquired, e.g., using array analysis either singly or in a library format. The database can be in substantially any form in which data can be maintained and transmitted, but is preferably an electronic database. The electronic database of the invention can be maintained on any electronic device allowing for the storage of and access to the database, such as a personal computer, but is preferably distributed on a wide area network, such as the World Wide Web.

The focus of the present section on databases that include peptide sequence data is for clarity of illustration only. It will be apparent to those of skill in the art that similar databases can be assembled for any assay data acquired using an assay of the invention.

The compositions and methods for identifying and/or quantitating the relative and/or absolute abundance of a variety of molecular and macromolecular species from a biological sample undergoing angiogenesis, i.e., the identification of angiogenesis-associated sequences described herein, provide an abundance of information, which can be correlated with pathological conditions, predisposition to disease, drug testing, therapeutic monitoring, gene-disease causal linkages, identification of correlates of immunity and physiological status, among others. Although the data generated from the assays of the invention is suited for manual review and analysis, in a preferred embodiment, prior data processing using high-speed computers is utilized.

An array of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which can be viewed as a tree structure or as the merger of two or more such tree structures.

The present invention provides a computer database comprising a computer and software for storing in computer-retrievable form assay data records cross-tabulated, e.g., with data specifying the source of the target-containing sample from which each sequence specificity record was obtained.

In an exemplary embodiment, at least one of the sources of target-containing sample is from a control tissue sample known to be free of pathological disorders. In a variation, at least one of the sources is a known pathological tissue specimen, e.g., a neoplastic lesion or another tissue specimen to be analyzed for angiogenesis. In another variation, the assay records cross-tabulate one or more of the following parameters for each target species in a sample: (1) a unique identification code, which can include, e.g., a target molecular structure and/or characteristic separation coordinate (e.g., electrophoretic coordinates); (2) sample source; and (3) absolute and/or relative quantity of the target species present in the sample.

The invention also provides for the storage and retrieval of a collection of target data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a protein expression fingerprint record comprising unique identifiers for at least 10 target data records cross-tabulated with target source.

When the target is a peptide or nucleic acid, the invention preferably provides a method for identifying related peptide or nucleic acid sequences, comprising performing a computerized comparison between a peptide or nucleic acid sequence assay record stored in or retrieved from a computer storage device or database and at least one other sequence. The comparison can include a sequence analysis or comparison algorithm or computer program embodiment thereof (e.g., FASTA, TFASTA, GAP, BESTFIT) and/or the comparison may be of the relative amount of a peptide or nucleic acid sequence in a pool of sequences determined from a polypeptide or nucleic acid sample of a specimen.

The invention also preferably provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 10BaseT), telephone line, ISDN line, wireless network, optical fiber, or other suitable signal tranmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

In a preferred embodiment, the invention provides a computer system for comparing a query target to a database containing an array of data structures, such as an assay result obtained by the method of the invention, and ranking database targets based on the degree of identity and gap weight to the target data. A central processor is preferably initialized to load and execute the computer program for alignment and/or comparison of the assay results. Data for a query target is entered into the central processor via an I/O device. Execution of the computer program results in the central processor retrieving the assay data from the data file, which comprises a binary description of an assay result.

The target data or record and the computer program can be transferred to secondary memory, which is typically random access memory (e.g., DRAM, SRAM, SGRAM, or SDRAM). Targets are ranked according to the degree of correspondence between a selected assay characteristic (e.g., binding to a selected affinity moiety) and the same characteristic of the query target and results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

The invention also preferably provides the use of a computer system, such as that described above, which comprises: (1) a computer; (2) a stored bit pattern encoding a collection of peptide sequence specificity records obtained by the methods of the invention, which may be stored in the computer; (3) a comparison target, such as a query target; and (4) a program for alignment and comparison, typically with rank-ordering of comparison results on the basis of computed similarity values.

Angiogenesis-associated Sequences

Angiogenesis proteins of the present invention may be classified as secreted proteins, transmembrane proteins or intracellular proteins. In one embodiment, the angiogenesis protein is an intracellular protein. Intracellular proteins may be found in the cytoplasm and/or in the nucleus or associated with the intracellular side of the plasma membrane. Intracellular proteins are involved in all aspects of cellular function and replication (including, e.g., signaling pathways); aberrant expression of such proteins often results in unregulated or disregulated cellular processes (see, e.g., Molecular Biology of the Cell, 3rd Edition, Alberts, Ed., Garland Pub., 1994). For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterizing proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs can be identified on the basis of primary sequence; thus, an analysis of the sequence of proteins may provide insight into both the enzymatic potential of the molecule and/or molecules with which the protein may associate.

In another embodiment, the angiogenesis sequences are transmembrane proteins. Transmembrane proteins are molecules that span a phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels and adenylyl cyclases contain numerous transmembrane domains. Many important cell surface receptors such as G protein coupled receptors (GPCRs) are classified as “seven transmembrane domain” proteins, as they contain 7 membrane spanning regions. Characteristics of transmembrane domains include approximately 20 consecutive hydrophobic amino acids that may be followed or flanked by charged amino acids. Therefore, upon analysis of the amino acid sequence of a particular protein, the localization and number of transmembrane domains within the protein may be predicted (see, e.g. PSORT web site http://psort.nibb.ac.jp/).

The extracellular domains of transmembrane proteins are diverse; however, conserved motifs are found repeatedly among various extracellular domains. Conserved structure and/or functions have been ascribed to different extracellular motifs. Many extracellular domains are involved in binding to other molecules. In one aspect, extracellular domains are found on receptors. Factors that bind the receptor domain include circulating ligands, which may be peptides, proteins, or small molecules such as adenosine and the like. For example, growth factors such as EGF, FGF and PDGF are circulating growth factors that bind to their cognate receptors to initiate a variety of cellular responses. Other factors include cytokines, mitogenic factors, neurotrophic factors and the like. Extracellular domains also bind to cell-associated molecules. In this respect, they mediate cell-cell interactions. Cell-associated ligands can be tethered to the cell for example via a glycosylphosphatidylinositol (GPI) anchor, or may themselves be transmembrane proteins. Extracellular domains also associate with the extracellular matrix and contribute to the maintenance of the cell structure.

Angiogenesis proteins that are transmembrane are particularly preferred in the present invention as they are readily accessible targets for immunotherapeutics, as are described herein. In addition, as outlined below, transmembrane proteins can be also useful in imaging modalities. Antibodies may be used to label such readily accessible proteins in situ. Alternatively, antibodies can also label intracellular proteins, in which case samples are typically permeablized to provide acess to intracellular proteins.

It will also be appreciated by those in the art that a transmembrane protein can be made soluble by removing transmembrane sequences, for example through recombinant methods. Furthermore, transmembrane proteins that have been made soluble can be made to be secreted through recombinant means by adding an appropriate signal sequence.

In another embodiment, the angiogenesis proteins are secreted proteins; the secretion of which can be either constitutive or regulated. These proteins have a signal peptide or signal sequence that targets the molecule to the secretory pathway. Secreted proteins are involved in numerous physiological events; by virtue of their circulating nature, they serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. Angiogenesis proteins that are secreted proteins are particularly preferred in the present invention as they serve as good targets for diagnostic markers, e.g., for blood or serum tests.

An angiogenesis sequence is typically initially identified by substantial nucleic acid and/or amino acid sequence homology or linkage to the angiogenesis sequences outlined herein. Such homology can be based upon the overall nucleic acid or amino acid sequence, and is generally determined as outlined below, using either homology programs or hybridization conditions. Typically, linked sequences on a mRNA are found on the same molecule.

As detailed in the definitions, percent identity can be determined using an algorithm such as BLAST. A preferred method utilizes the BLASTN module of WU-BLAST-2 set to the default parameters, with overlap span and overlap fraction set to 1 and 0.125, respectively. The alignment may include the introduction of gaps in the sequences to be aligned. In addition, for sequences which contain either more or fewer nucleotides than those of the nucleic acids of the figures, it is understood that the percentage of homology will be determined based on the number of homologous nucleosides in relation to the total number of nucleosides. Thus, for example, homology of sequences shorter than those of the sequences identified herein and as discussed below, will be determined using the number of nucleosides in the shorter sequence.

In one embodiment, the nucleic acid homology is determined through hybridization studies. Thus, e.g., nucleic acids which hybridize under high stringency to a nucleic acid of Tables 1-8, or its complement, or is also found on naturally occurring mRNAs is considered an angiogenesis sequence. In another embodiment, less stringent hybridization conditions are used; for example, moderate or low stringency conditions may be used, as are known in the art; see Ausubel, supra, and Tijssen, supra.

In addition, the angiogenesis nucleic acid sequences of the invention, e.g, the sequence in Tables 1-8, are fragments of larger genes, i.e. they are nucleic acid segments. “Genes” in this context includes coding regions, non-coding regions, and mixtures of coding and non-coding regions. Accordingly, as will be appreciated by those in the art, using the sequences provided herein, extended sequences, in either direction, of the angiogenesis genes can be obtained, using techniques well known in the art for cloning either longer sequences or the full length sequences; see Ausubel, et al., supra. Much can be done by informatics and many sequences can be clustered to include multiple sequences, e.g., systems such as UniGene (see, http://www.ncbi.nlm.nih.gov/UniGene/).

Once the angiogenesis nucleic acid is identified, it can be cloned and, if necessary, its constituent parts recombined to form the entire angiogenesis nucleic acid coding regions or the entire mRNA sequence. Once isolated from its natural source, e.g., contained within a plasmid or other vector or excised therefrom as a linear nucleic acid segment, the recombinant angiogenesis nucleic acid can be further-used as a probe to identify and isolate other angiogenesis nucleic acids, for example extended coding regions. It can also be used as a “precursor” nucleic acid to make modified or variant angiogenesis nucleic acids and proteins.

The angiogenesis nucleic acids of the present invention are used in several ways. In a first embodiment, nucleic acid probes to the angiogenesis nucleic acids are made and attached to biochips to be used in screening and diagnostic methods, as outlined below, or for administration, for example for gene therapy, vaccine, and/or antisense applications. Alternatively, the angiogenesis nucleic acids that include coding regions of angiogenesis proteins can be put into expression vectors for the expression of angiogenesis proteins, again for screening purposes or for administration to a patient.

In a preferred embodiment, nucleic acid probes to angiogenesis nucleic acids (both the nucleic acid sequences outlined in the figures and/or the complements thereof) are made. The nucleic acid probes attached to the biochip are designed to be substantially complementary to the angiogenesis nucleic acids, i.e. the target sequence (either the target sequence of the sample or to other probe sequences, for example in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. That is, generally whole genes are not used. In some embodiments, much longer nucleic acids can be used, up to hundreds of bases.

In a preferred embodiment, more than one probe per sequence is used, with either overlapping probes or probes to different sections of the target being used. That is, two, three, four or more probes, with three being preferred, are used to build in a redundancy for a particular target. The probes can be overlapping (i.e. have some sequence in common), or separate. In some cases, PCR primers may be used to amplify signal for higher sensitivity.

As will be appreciated by those in the art, nucleic acids can be attached or immobilized to a solid support in a wide variety of ways. By “immobilized” and grammatical equivalents herein is meant the association or binding between the nucleic acid probe and the solid support is sufficient to be stable under the conditions of binding, washing, analysis, and removal as outlined below. The binding can typically be covalent or non-covalent. By “non-covalent binding” and grammatical equivalents herein is meant one or more of electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant a material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses, plastics, etc. In general, the substrates allow optical detection and do not appreciably fluorescese. A preferred substrate is described in copending application entitled Reusable Low Fluorescent Plastic Biochip, U.S. application Ser. No. 09/270,214, filed Mar. 15, 1999, herein incorporated by reference in its entirety.

Generally the substrate is planar, although as will be appreciated by those in the art, other configurations of substrates may be used as well. For example, the probes may be placed on the inside surface of a tube, for flow-through sample analysis to minimize sample volume. Similarly, the substrate may be flexible, such as a flexible foam, including closed cell foams made of particular plastics.

In a preferred embodiment, the surface of the biochip and the probe may be derivatized with chemical functional groups for subsequent attachment of the two. Thus, for example, the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, for example using linkers as are known in the art; for example, homo- or hetero-bifunctional linkers as are well known (see 1994 Pierce Chemical Company catalog, technical section on cross-linkers, pages 155-200, incorporated herein by reference). In addition, in some cases, additional linkers, such as alkyl groups (including substituted and heteroalkyl groups) may be used.

In this embodiment, oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

In another embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques, such as those described in WO 95/25116; WO 95/35505; U.S. Pat. Nos. 5,700,637 and 5,445,934; and references cited within, all of which are expressly incorporated by reference; these methods of attachment form the basis of the Affimetrix GeneChip™ technology.

Often, amplification-based assays are performed to measure the expression level of angiogenesis-associated sequences. These assays are typically performed in conjunction with reverse transcription. In such assays, an angiogenesis-associated nucleic acid sequence acts as a template in an amplification reaction (e.g., Polymerase Chain Reaction, or PCR). In a quantitative amplification, the amount of amplification product will be proportional to the amount of template in the original sample. Comparison to appropriate controls provides a measure of the amount of angiogenesis-associated RNA. Methods of quantitative amplification are well known to those of skill in the art. Detailed protocols for quantitative PCR are provided, e.g., in Innis et al. (1990) PCR Protocols, A Guide to Methods and Applications, Academic Press, Inc. N.Y.).

In some embodiments, a TaqMan based assay is used to measure expression. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, for example, literature provided by Perkin-Elmer, e.g., www2.perkin-elmer.com).

Other suitable amplification methods include, but are not limited to, ligase chain reaction (LCR) (see, Wu and Wallace (1989) Genomics 4: 560, Landegren et al. (1988) Science 241: 1077, and Barringer et al. (1990) Gene 89: 117), transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

In a preferred embodiment, angiogenesis nucleic acids, e.g., encoding angiogenesis proteins are used to make a variety of expression vectors to express angiogenesis proteins which can then be used in screening assays, as described below. Expression vectors and recombinant DNA technology are well known to those of skill in the art (see, e.g., Ausubel, supra, and Gene Expression Systems, Fernandez & Hoeffler, Eds, Academic Press, 1999) and are used to express proteins. The expression vectors may be either self-replicating extrachromosomal vectors or vectors which integrate into a host genome. Generally, these expression vectors include transcriptional and translational regulatory nucleic acid operably linked to the nucleic acid encoding the angiogenesis protein. The term “control sequences” refers to DNA sequences used for the expression of an operably linked coding sequence in a particular host organism. Control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is typically accomplished by ligation at convenient restriction sites. If such sites do not exist, synthetic oligonucleotide adaptors or linkers are used in accordance with conventional practice. Transcriptional and translational regulatory nucleic acid will generally be appropriate to the host cell used to express the angiogenesis protein; for example, transcriptional and translational regulatory nucleic acid sequences from Bacillus are preferably used to express the angiogenesis protein in Bacillus. Numerous types of appropriate expression vectors, and suitable regulatory sequences are known in the art for a variety of host cells.

In general, transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, an expression vector may comprise additional elements. For example, the expression vector may have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art (e.g., Fernandez & Hoeffler, supra). See also Kitamura, et al. (1995) PNAS 92:9146-9150.

In addition, in a preferred embodiment, the expression vector contains a selectable marker gene to allow the selection of transformed host cells. Selection genes are well known in the art and will vary with the host cell used.

The angiogenesis proteins of the present invention are produced by culturing a host cell transformed with an expression vector containing nucleic acid encoding an angiogenesis protein, under the appropriate conditions to induce or cause expression of the angiogenesis protein. Conditions appropriate for angiogenesis protein expression will vary with the choice of the expression vector and the host cell, and will be easily ascertained by one skilled in the art through routine experimentation or optimization. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect and animal cells, including mammalian cells. Of particular interest are Saccharomyces cerevisiae and other yeasts, E. coli, Bacillus subtilis, Sf9 cells, C129 cells, 293 cells, Neurospora, BHK, CHO, COS, HeLa cells, HUVEC (human umbilical vein endothelial cells), THP1 cells (a macrophage cell line) and various other human cells and cell lines.

In a preferred embodiment, the angiogenesis proteins are expressed in mammalian cells. Mammalian expression systems are also known in the art, and include retroviral and adenoviral systems. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter (see, e.g., Fernandez & Hoeffler, supra). Typically, transcription termination and polyadenylation sequences recognized by mammalian cells are regulatory regions located 3′ to the translation stop codon and thus, together with the promoter elements, flank the coding sequence. Examples of transcription terminator and polyadenlytion signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, angiogenesis proteins are expressed in bacterial systems. Bacterial expression systems are well known in the art. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the angiogenesis protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others (e.g., Fernandez & Hoeffler, supra). The bacterial expression vectors are transformed into bacterial host cells using techniques well known in the art, such as calcium chloride treatment, electroporation, and others.

In one embodiment, angiogenesis proteins are produced in insect cells. Expression vectors for the transformation of insect cells, and in particular, baculovirus-based expression vectors, are well known in the art.

In a preferred embodiment, angiogenesis protein is produced in yeast cells. Yeast expression systems are well known in the art, and include expression vectors for Saccharomyces cerevisiae, Candida albicans and C. maltosa, Hansenula polymrorpha, Kluyveromyces fragilis and K. lactis, Pichia guillerimondii and P. pastoris, Schizosaccharomyces pombe, and Yarrowia lipolytica.

The angiogenesis protein may also be made as a fusion protein, using techniques well known in the art. Thus, for example, for the creation of monoclonal antibodies, if the desired epitope is small, the angiogenesis protein may be fused to a carrier protein to form an immunogen. Alternatively, the angiogenesis protein may be made as a fusion protein to increase expression, or for other reasons. For example, when the angiogenesis protein is an angiogenesis peptide, the nucleic acid encoding the peptide may be linked to another nucleic acid for expression purposes. Fusion with detection epitope tags can be made, e.g., with FLAG, His 6, myc, HA, etc.

In one embodiment, the angiogenesis nucleic acids, proteins and antibodies of the invention are labeled. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies, antigens, or epitope tags and c) colored or fluorescent dyes. The labels may be incorporated into the angiogenesis nucleic acids, proteins and antibodies at any position. For example, the label should be capable of producing, either directly or indirectly, a detectable signal. The detectable moiety may be a radioisotope, such as ³H, ¹⁴C, ³²p, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Accordingly, the present invention also provides angiogenesis protein sequences. An angiogenesis protein of the present invention may be identified in several ways. “Protein” in this sense includes proteins, polypeptides, and peptides. As will be appreciated by those in the art, the nucleic acid sequences of the invention can be used to generate protein sequences. There are a variety of ways to do this, including cloning the entire gene and verifying its frame and amino acid sequence, or by comparing it to known sequences to search for homology to provide a frame, assuming the angiogenesis protein has an identifiable motif or homology to some protein in the database being used. Generally, the nucleic acid sequences are input into a program that will search all three frames for homology. This is done in a preferred embodiment using the following NCBI Advanced BLAST parameters. The program is blastx or blastn. The database is nr. The input data is as “Sequence in FASTA format”. The organism list is “none”. The “expect” is 10; the filter is default. The “descriptions” is 500, the “alignments” is 500, and the “alignment view” is pairwise. The “Query Genetic Codes” is standard (1). The matrix is BLOSUM62; gap existence cost is 11, per residue gap cost is 1; and the lambda ratio is 0.85 default. This results in the generation of a putative protein sequence.

Also included within one embodiment of angiogenesis proteins are amino acid variants of the naturally occurring sequences, as determined herein. Preferably, the variants are preferably greater than about 75% homologous to the wild-type sequence, more preferably greater than about 80%, even more preferably greater than about 85% and most preferably greater than 90%. In some embodiments the homology will be as high as about 93 to 95 or 98%. As for nucleic acids, homology in this context means sequence similarity or identity, with identity being preferred. This homology will be determined using standard techniques well known in the art as are outlined above for the nucleic acid homologies.

Angiogenesis proteins of the present invention may be shorter or longer than the wild type amino acid sequences. Thus, in a preferred embodiment, included within the definition of angiogenesis proteins are portions or fragments of the wild type sequences. herein. In addition, as outlined above, the angiogenesis nucleic acids of the invention may be used to obtain additional coding regions, and thus additional protein sequence, using techniques known in the art.

In a preferred embodiment, the angiogenesis proteins are derivative or variant angiogenesis proteins as compared to the wild-type sequence. That is, as outlined more fully below, the derivative angiogenesis peptide will often contain at least one amino acid substitution, deletion or insertion, with amino acid substitutions being particularly preferred. The amino acid substitution, insertion or deletion may occur at any residue within the angiogenesis peptide.

Also included within one embodiment of angiogenesis proteins of the present invention are amino acid sequence variants. These variants typically fall into one or more of three classes: substitutional, insertional or deletional variants. These variants ordinarily are prepared by site specific mutagenesis of nucleotides in the DNA encoding the angiogenesis protein, using cassette or PCR mutagenesis or other techniques well known in the art, to produce DNA encoding the variant, and thereafter expressing the DNA in recombinant cell culture as outlined above. However, variant angiogenesis protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the angiogenesis protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed angiogenesis variants screened for the optimal combination of desired activity. Techniques for making substitution mutations at predetermined sites in DNA having a known sequence are well known, for example, M13 primer mutagenesis and PCR mutagenesis. Screening of the mutants is done using assays of angiogenesis protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the angiogenesis protein are desired, substitutions are generally made in accordance with the amino acid substitution chart provided in the definition section.

Substantial changes in function or immunological identity are made by selecting substitutions that are less conservative than those provided in the definition of “conservative substitution”. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analog, although variants also are selected to modify the characteristics of the angiogenesis proteins as needed. Alternatively, the variant may be designed such that the biological activity of the angiogenesis protein is altered. For example, glycosylation sites may be altered or removed.

Covalent modifications of angiogenesis polypeptides are included within the scope of this invention. One type of covalent modification includes reacting targeted amino acid residues of an angiogenesis polypeptide with an organic derivatizing agent that is capable of reacting with selected side chains or the N- or C-terminal residues of an angiogenesis polypeptide. Derivatization with bifunctional agents is useful, for instance, for crosslinking angiogenesis polypeptides to a water-insoluble support matrix or surface for use in the method for purifying anti-angiogenesis polypeptide antibodies or screening assays, as is more fully described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl, threonyl or tyrosyl residues, methylation of the γ-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of the angiogenesis polypeptide included within the scope of this invention comprises altering the native glycosylation pattern of the polypeptide. “Altering the native glycosylation pattern” is intended for purposes herein to mean deleting one or more carbohydrate moieties found in native sequence angiogenesis polypeptide, and/or adding one or more glycosylation sites that are not present in the native sequence angiogenesis polypeptide. Glycosylation patterns can be altered in many ways. For example the use of different cell types to express angiogenesis-associated sequences can result in different glycosylation patterns.

Addition of glycosylation sites to angiogenesis polypeptides may also be accomplished by altering the amino acid sequence thereof. The alteration may be made, for example, by the addition of, or substitution by, one or more serine or threonine residues to the native sequence angiogenesis polypeptide (for O-linked glycosylation sites). The angiogenesis amino acid sequence may optionally be altered through changes at the DNA level, particularly by mutating the DNA encoding the angiogenesis polypeptide at preselected bases such that codons are generated that will translate into the desired amino acids.

Another means of increasing the number of carbohydrate moieties on the angiogenesis polypeptide is by chemical or enzymatic coupling of glycosides to the polypeptide. Such methods are described in the art, e.g., in WO 87/05330 published Sep. 11, 1987, and in Aplin and Wriston, CRC Crit. Rev. Biochem., pp. 259-306 (1981).

Removal of carbohydrate moieties present on the angiogenesis polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of angiogenesis comprises linking the angiogenesis polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Angiogenesis polypeptides of the present invention may also be modified in a way to form chimeric molecules comprising an angiogenesis polypeptide fused to another, heterologous polypeptide or amino acid sequence. In one embodiment, such a chimeric molecule comprises a fusion of an angiogenesis polypeptide with a tag polypeptide which provides an epitope to which an anti-tag antibody can selectively bind. The epitope tag is generally placed at the amino-or carboxyl-terminus of the angiogenesis polypeptide. The presence of such epitope-tagged forms of an angiogenesis polypeptide can be detected using an antibody against the tag polypeptide. Also, provision of the epitope tag enables the angiogenesis polypeptide to be readily purified by affinity purification using an anti-tag antibody or another type of affinity matrix that binds to the epitope tag. In an alternative embodiment, the chimeric molecule may comprise a fusion of an angiogenesis polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; HIS6 and metal chelation tags, the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

Also included with an embodiment of angiogenesis protein are other angiogenesis proteins of the angiogenesis family, and angiogenesis proteins from other organisms, which are cloned and expressed as outlined below. Thus, probe or degenerate polymerase chain reaction (PCR) primer sequences may be used to find other related angiogenesis proteins from humans or other organisms. As will be appreciated by those in the art, particularly useful probe and/or PCR primer sequences include the unique areas of the angiogenesis nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art (e.g., Innis, PCR Protocols, supra).

In addition, as is outlined herein, angiogenesis proteins can be made that are longer than those encoded by the nucleic acids of the figures, e.g., by the elucidation of extended sequences, the addition of epitope or purification tags, the addition of other fusion sequences, etc.

Angiogenesis proteins may also be identified as being encoded by angiogenesis nucleic acids. Thus, angiogenesis proteins are encoded by nucleic acids that will hybridize to the sequences of the sequence listings, or their complements, as outlined herein.

In a preferred embodiment, when the angiogenesis protein is to be used to generate antibodies, e.g., for immunotherapy or immunodiagnosis, the angiogenesis protein should share at least one epitope or determinant with the full length protein. By “epitope” or “determinant” herein is typically meant a portion of a protein which will generate and/or bind an antibody or T-cell receptor in the context of MHC. Thus, in most instances, antibodies made to a smaller angiogenesis protein will be able to bind to the full-length protein, particularly linear epitopes. In a preferred embodiment, the epitope is unique; that is, antibodies generated to a unique epitope show little or no cross-reactivity. In a preferred embodiment, the epitope is selected from a protein sequence set out in Table 8.

Methods of preparing polyclonal antibodies are known to the skilled artisan (e.g., Coligan, supra; and Harlow & Lane, supra). Polyclonal antibodies can be raised in a mammal, e.g., by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the figures or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Tables 1-8, or fragment thereof, or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are monoclonal, preferably human or humanized, antibodies that have binding specificities for at least two different antigens or that have binding specificities for two epitopes on the same antigen. In one embodiment, one of the binding specificities is for a protein encoded by a nucleic acid Tables 1-8 or a fragment thereof, the other one is for any other antigen, and preferably for a cell-surface protein or receptor or receptor subunit, preferably one that is tumor specific. Alternatively, tetramer-type technology may create multivalent reagents.

In a preferred embodiment, the antibodies to angiogenesis protein are capable of reducing or eliminating a biological function of an angiogenesis protein, as is described below. That is, the addition of anti-angiogenesis protein antibodies (either polyclonal or preferably monoclonal) to angiogenic tissue (or cells containing angiogenesis) may reduce or eliminate the angiogenesis activity. Generally, at least a 25% decrease in activity is preferred, with at least about 50% being particularly preferred and about a 95-100% decrease being especially preferred.

In a preferred embodiment the antibodies to the angiogenesis proteins are humanized antibodies (e.g., Xenerex Biosciences, Mederex, Inc., Abgenix, Inc., Protein Design Labs, Inc.) Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

By immunotherapy is meant treatment of angiogenesis with an antibody raised against angiogenesis proteins. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen, leading to an immune response.

In a preferred embodiment the angiogenesis proteins against which antibodies are raised are secreted proteins as described above. Without being bound by theory, antibodies used for treatment, bind and prevent the secreted protein from binding to its receptor, thereby inactivating the secreted angiogenesis protein.

In another preferred embodiment, the angiogenesis protein to which antibodies are raised is a transmembrane protein. Without being bound by theory, antibodies used for treatment, bind the extracellular domain of the angiogenesis protein and prevent it from binding to other proteins, such as circulating ligands or cell-associated molecules. The antibody may cause down-regulation of the transmembrane angiogenesis protein. As will be appreciated by one of ordinary skill in the art, the antibody may be a competitive, non-competitive or uncompetitive inhibitor of protein binding to the extracellular domain of the angiogenesis protein. The antibody is also an antagonist of the angiogenesis protein. Further, the antibody prevents activation of the transmembrane angiogenesis protein. In one aspect, when the antibody prevents the binding of other molecules to the angiogenesis protein, the antibody prevents growth of the cell. The antibody may also be used to target or sensitize the cell to cytotoxic agents, including, but not limited to TNF-α, TNF-β, IL-1, INF-γ and IL-2, or chemotherapeutic agents including 5FU, vinblastine, actinomycin D, cisplatin, methotrexate, and the like. In some instances the antibody belongs to a sub-type that activates serum complement when complexed with the transmembrane protein thereby mediating cytotoxicity or antigen-dependent cytotoxicity (ADCC). Thus, angiogenesis is treated by administering to a patient antibodies directed against the transmembrane angiogenesis protein. Antibody-labeling may activate a co-toxin, localize a toxin payload, or otherwise provide means to locally ablate cells.

In another preferred embodiment, the antibody is conjugated or fused to an effector moiety. The effector moiety can be any number of molecules, including labelling moieties such as radioactive labels or fluorescent labels, or can be a therapeutic moiety. In one aspect the therapeutic moiety is a small molecule that modulates the activity of the angiogenesis protein. In another aspect the therapeutic moiety modulates the activity of molecules associated with or in close proximity to the angiogenesis protein. The therapeutic moiety may inhibit enzymatic activity such as protease or collagenase activity associated with angiogenesis, or be an attractant of other cells, such as NK cells.

In a preferred embodiment, the therapeutic moiety can also be a cytotoxic agent. In this method, targeting the cytotoxic agent to angiogenesis tissue or cells, results in a reduction in the number of afflicted cells, thereby reducing symptoms associated with angiogenesis. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against angiogenesis proteins, or binding of a radionuclide to a chelating agent that has been covalently attached to the antibody. Targeting the therapeutic moiety to transmembrane angiogenesis proteins not only serves to increase the local concentration of therapeutic moiety in the angiogenesis afflicted area, but also serves to reduce deleterious side effects that may be associated with the therapeutic moiety.

In another preferred embodiment, the angiogenesis protein against which the antibodies are raised is an intracellular protein. In this case, the antibody may be conjugated or fused to a protein which facilitates entry into the cell. In one case, the antibody enters the cell by endocytosis. In another embodiment, a nucleic acid encoding the antibody is administered to the individual or cell. Moreover, wherein the angiogenesis protein can be targeted within a cell, i.e., the nucleus, an antibody thereto contains a signal for that target localization, i.e., a nuclear localization signal.

The angiogenesis antibodies of the invention specifically bind to angiogenesis proteins. By “specifically bind” herein is meant that the antibodies bind to the protein with a K_{d} of at least about 0.1 mM, more usually at least about 1 μM, preferably at least about 0.1 μM or better, and most preferably, 0.01 μM or better. Selectivity of binding is also important.

In a preferred embodiment, the angiogenesis protein is purified or isolated after expression. Angiogenesis proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the angiogenesis protein may be purified using a standard anti-angiogenesis protein antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the angiogenesis protein. In some instances no purification will be necessary.

Once expressed and purified if necessary, the angiogenesis proteins and nucleic acids are useful in a number of applications. They may be used as immunoselection reagents, as vaccine reagents, as screening agents, etc.

Detection of Angiogenesis Sequence for Diagnostic and Therapeutic Applications

In one aspect, the RNAexpression levels of genes are determined for different cellular states in the angiogenesis phenotype. Expression levels of genes in normal tissue (i.e., not undergoing angiogenesis) and in angiogenesis tissue (and in some cases, for varying severities of angiogenesis that relate to prognosis, as outlined below) are evaluated to provide expression profiles. An expression profile of a particular cell state or point of development is essentially a “fingerprint” of the state. While two states may have any particular gene similarly expressed, the evaluation of a number of genes simultaneously allows the generation of a gene expression profile that is reflective of the state of the cell. By comparing expression profiles of cells in different states, information regarding which genes are important (including both up- and down-regulation of genes) in each of these states is obtained. Then, diagnosis may be performed or confirmed to determine whether a tissue sample has the gene expression profile of normal or angiogenesic tissue. This will provide for molecular diagnosis of related conditions.

“Differential expression,” or grammatical equivalents as used herein, refers to qualitative or quantitative differences in the temporal and/or cellular gene expression patterns within and among cells and tissue. Thus, a differentially expressed gene can qualitatively have its expression altered, including an activation or inactivation, in, e.g., normal versus angiogenic tissue. Genes may be turned on or turned off in a particular state, relative to another state thus permitting comparison of two or more statese. A qualitatively regulated gene will exhibit an expression pattern within a state or cell type which is detectable by standard techniques. Some genes will be expressed in one state or cell type, but not in both. Alternatively, the difference in expression may be quantitative, e.g., in that expression is increased or decreased; i.e., gene expression is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip™ expression arrays, Lockhart, Nature Biotechnology, 14:1675-1680 (1996), hereby expressly incorporated by reference. Other techniques include, but are not limited to, quantitative reverse transcriptase PCR, Northern analysis and RNase protection. As outlined above, preferably the change in expression (i.e., upregulation or downregulation) is at least about 50%, more preferably at least about 100%, more preferably at least about 150%, more preferably at least about 200%, with from 300 to at least 1000% being especially preferred.

Evaluation may be at the gene transcript, or the protein level. The amount of gene expression may be monitored using nucleic acid probes to the DNA or RNA equivalent of the gene transcript, and the quantification of gene expression levels, or, alternatively, the final gene product itself (protein) can be monitored, e.g., with antibodies to the angiogenesis protein and standard immunoassays (ELISAs, etc.) or other techniques, including mass spectroscopy assays, 2D gel electrophoresis assays, etc. Proteins corresponding to angiogenesis genes, i.e., those identified as being important in an angiogenesis phenotype, can be evaluated in an angiogenesis diagnostic test.

In a preferred embodiment, gene expression monitoring is performed simultaneously on a number of genes. Multiple protein expression monitoring can be performed as well. Similarly, these assays may be performed on an individual basis as well.

In this embodiment, the angiogenesis nucleic acid probes are attached to biochips as outlined herein for the detection and quantification of angiogenesis sequences in a particular cell. The assays are further described below in the example. PCR techniques can be used to provide greater sensitivity.

In a preferred embodiment nucleic acids encoding the angiogenesis protein are detected. Although DNA or RNA encoding the angiogenesis protein may be detected, of particular interest are methods wherein an mRNA encoding an angiogenesis protein is detected. Probes to detect mRNA can be a nucleotide/deoxynucleotide probe that is complementary to and hybridizes with the mRNA and includes, but is not limited to, oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding an angiogenesis protein is detected by binding the digoxygenin with an anti-digoxygenin secondary antibody and developed with nitro blue tetrazolium and 5-bromo-4-chloro-3-indoyl phosphate.

In a preferred embodiment, various proteins from the three classes of proteins as described herein (secreted, transmembrane or intracellular proteins) are used in diagnostic assays. The angiogenesis proteins, antibodies, nucleic acids, modified proteins and cells containing angiogenesis sequences are used in diagnostic assays. This can be performed on an individual gene or corresponding polypeptide level. In a preferred embodiment, the expression profiles are used, preferably in conjunction with high throughput screening techniques to allow monitoring for expression profile genes and/or corresponding polypeptides.

As described and defined herein, angiogenesis proteins, including intracellular, transmembrane or secreted proteins, find use as markers of angiogenesis. Detection of these proteins in putative angiogenesis tissue allows for detection or diagnosis of angiogenesis. In one embodiment, antibodies are used to detect angiogenesis proteins. A preferred method separates proteins from a sample by electrophoresis on a gel (typically a denaturing and reducing protein gel, but may be another type of gel, including isoelectric focusing gels and the like). Following separation of proteins, the angiogenesis protein is detected, e.g., by immunoblotting with antibodies raised against the angiogenesis protein. Methods of immunoblotting are well known to those of ordinary skill in the art.

In another preferred method, antibodies to the angiogenesis protein find use in in situ imaging techniques, e.g., in histology (e.g., Methods in Cell Biology: Antibodies in Cell Biology, volume 37 (Asai, ed. 1993)). In this method cells are contacted with from one to many antibodies to the angiogenesis protein(s). Following washing to remove non-specific antibody binding, the presence of the antibody or antibodies is detected. In one embodiment the antibody is detected by incubating with a secondary antibody that contains a detectable label. In another method the primary antibody to the angiogenesis protein(s) contains a detectable label, for example an enzyme marker that can act on a substrate. In another preferred embodiment each one of multiple primary antibodies contains a distinct and detectable label. This method finds particular use in simultaneous screening for a plurality of angiogenesis proteins. As will be appreciated by one of ordinary skill in the art, many other histological imaging techniques are also provided by the invention.

In a preferred embodiment the label is detected in a fluorometer which has the ability to detect and distinguish emissions of different wavelengths. In addition, a fluorescence activated cell sorter (FACS) can be used in the method.

In another preferred embodiment, antibodies find use in diagnosing angiogenesis from biological samples, such as blood, urine, sputum, or other bodily fluids. As previously described, certain angiogenesis proteins are secreted/circulating molecules. Blood samples, therefore, are useful as samples to be probed or tested for the presence of secreted angiogenesis proteins. Antibodies can be used to detect an angiogenesis protein by previously described immunoassay techniques including ELISA, immunoblotting (Western blotting), immunoprecipitation, BIACORE technology and the like. Conversely, the presence of antibodies may indicate an immune response against an endogenous angiogenesis protein.

In a preferred embodiment, in situ hybridization of labeled angiogenesis nucleic acid probes to tissue arrays is done. For example, arrays of tissue samples, including angiogenesis tissue and/or normal tissue, are made. In situ hybridization (see, e.g., Ausubel, supra) is then performed. When comparing the fingerprints between an individual and a standard, the skilled artisan can make a diagnosis, a prognosis, or a prediction based on the findings. It is further understood that the genes which indicate the diagnosis may differ from those which indicate the prognosis and molecular profiling of the condition of the cells may lead to distinctions between responsive or refractory conditions or may be predictive of outcomes.

In a preferred embodiment, the angiogenesis proteins, antibodies, nucleic acids, modified proteins and cells containing angiogenesis sequences are used in prognosis assays. As above, gene expression profiles can be generated that correlate to angiogenesis severity, in terms of long term prognosis. Again, this may be done on either a protein or gene level, with the use of genes being preferred. As above, angiogenesis probes may be attached to biochips for the detection and quantification of angiogenesis sequences in a tissue or patient. The assays proceed as outlined above for diagnosis. PCR method may provide more sensitive and accurate quantification.

In a preferred embodiment members of the three classes of proteins as described herein are used in drug screening assays. The angiogenesis proteins, antibodies, nucleic acids, modified proteins and cells containing angiogenesis sequences are used in drug screening assays or by evaluating the effect of drug candidates on a “gene expression profile” or expression profile of polypeptides. In a preferred embodiment, the expression profiles are used, preferably in conjunction with high throughput screening techniques to allow monitoring for expression profile genes after treatment with a candidate agent (e.g., Zlokarnik, et al., Science 279, 84-8 (1998); Heid, Genome Res 6:986-94, 1996).

In a preferred embodiment, the angiogenesis proteins, antibodies, nucleic acids, modified proteins and cells containing the native or modified angiogenesis proteins are used in screening assays. That is, the present invention provides novel methods for screening for compositions which modulate the angiogenesis phenotype or an identified physiological function of an angiogenesis protein. As above, this can be done on an individual gene level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the differentially expressed genes herein, a variety of assays may be executed. In a preferred embodiment, assays may be run on an individual gene or protein level. That is, having identified a particular gene as up regulated in angiogenesis, test compounds can be screened for the ability to modulate gene expression or for binding to the angiogenic protein. “Modulation” thus includes both an increase and a decrease in gene expression. The preferred amount of modulation will depend on the original change of the gene expression in normal versus tissue undergoing angiogenesis, with changes of at least 10%, preferably 50%, more preferably 100-300%, and in some embodiments 300-1000% or greater. Thus, if a gene exhibits a 4-fold increase in angiogenic tissue compared to normal tissue, a decrease of about four-fold is often desired; similarly, a 10-fold decrease in angiogenic tissue compared to normal tissue often provides a target value of a 10-fold increase in expression to be induced by the test compound.

The amount of gene expression may be monitored using nucleic acid probes and the quantification of gene expression levels, or, alternatively, the gene product itself can be monitored, e.g., through the use of antibodies to the angiogenesis protein and standard immunoassays. Proteomics and separation techniques may also allow quantification of expression.

In a preferred embodiment, gene expression or protein monitoring of a number of entitites, i.e., an expression profile, is monitored simultaneously. Such profiles will typically invove a plurality of those entitites described herein..

In this embodiment, the angiogenesis nucleic acid probes are attached to biochips as outlined herein for the detection and quantification of angiogenesis sequences in a particular cell. Alternatively, PCR may be used. Thus, a series, e.g., of microtiter plate, may be used with dispensed primers in desired wells. A PCR reaction can then be performed and analyzed for each well.

Modulators of Angiogenesis

Expression monitoring can be performed to identify compounds that modify the expression of one or more angiogenesis-associated sequences, e.g., a polynucleotide sequence set out in Tables 1-8. Generally, in a preferred embodiment, a test modulator is added to the cells prior to analysis. Moreover, screens are also provided to identify agents that modulate angiogenesis, modulate angiogenesis proteins, bind to an angiogenesis protein, or interfere with the binding of an angiogenesis protein and an antibody or other binding partner.

The term “test compound” or “drug candidate” or “modulator” or grammatical equivalents as used herein describes any molecule, e.g., protein, oligopeptide, small organic molecule, polysaccharide, polynucleotide, etc., to be tested for the capacity to directly or indirectly alter the angiogenesis phenotype or the expression of an angiogenesis sequence, e.g., a nucleic acid or protein sequence. In preferred embodiments, modulators alter expression profiles, or expression profile nucleic acids or proteins provided herein. In one embodiment, the modulator suppresses an angiogenesis phenotype, for example to a normal tissue fingerprint. In another embodiment, a modulator induced an angiogenesis phenotype. Generally, a plurality of assay mixtures are run in parallel with different agent concentrations to obtain a differential response to the various concentrations. Typically, one of these concentrations serves as a negative control, i.e., at zero concentration or below the level of detection.

In one aspect, a modulator will neutralize the effect of an angiogenesis protein. By “neutralize” is meant that activity of a protein is inhibited or blocked and thereby has substantially no effect on a cell.

In certain embodiments, combinatorial libraries of potential modulators will be screened for an ability to bind to an angiogenesis polypeptide or to modulate activity. Conventionally, new chemical entities with useful properties are generated by identifying a chemical compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et al. (1994) J. Med. Chem. 37(9): 1233-1251).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88), peptoids (PCT Publication No WO 91/19735, Dec. 26, 1991), encoded peptides (PCT Publication WO 93/20242, Oct. 14, 1993), random bio-oligomers (PCT Publication WO 92/00091, Jan. 9, 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083), antibody libraries (see, e.g., Vaughn et al. (1996) Nature Biotechnology, 14(3): 309-314), and PCT/US96/10287), carbohydrate libraries (see, e.g., Liang et al., (1996) Science, 274: 1520-1522, and U.S. Pat. No. 5,593,853), and small organic molecule libraries (see, e.g., benzodiazepines, Baum (1993) C&EN, January 18, page 33; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514; and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

The assays to identify modulators are amenable to high throughput screening. Preferred assays thus detect enhancement or inhibition of angiogenesis gene transcription, inhibition or enhancement of polypeptide expression, and inhibition or enhancement of polypeptide activity.

High throughput assays for the presence, absence, quantification, or other properties of particular nucleic acids or protein products are well known to those of skill in the art. Similarly, binding assays and reporter gene assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures, including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

In one embodiment, modulators are proteins, often naturally occurring proteins or fragments of naturally occurring proteins. Thus, e.g., cellular extracts containing proteins, or random or directed digests of proteinaceous cellular extracts, may be used. In this way libraries of proteins may be made for screening in the methods of the invention. Particularly preferred in this embodiment are libraries of bacterial, fungal, viral, and mammalian proteins, with the latter being preferred, and human proteins being especially preferred. Paticularly useful test compound will be directed to the class of proteins to which the target belongs, e.g., substrates for enzymes or ligands and receptors.

In a preferred embodiment, modulators are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

Modulators of angiogenesis can also be nucleic acids, as defined above.

As described above generally for proteins, nucleic acid modulating agents may be naturally occurring nucleic acids, random nucleic acids, or “biased” random nucleic acids. For example, digests of procaryotic or eucaryotic genomes may be used as is outlined above for proteins.

In a preferred embodiment, the candidate compounds are organic chemical moieties, a wide variety of which are available in the literature.

After the candidate agent has been added and the cells allowed to incubate for some period of time, the sample containing a target sequence to be analyzed is added to the biochip. If required, the target sequence is prepared using known techniques. For example, the sample may be treated to lyse the cells, using known lysis buffers, electroporation, etc., with purification and/or amplification such as PCR performed as appropriate. For example, an in vitro transcription with labels covalently attached to the nucleotides is performed. Generally, the nucleic acids are labeled with biotin-FITC or PE, or with cy3 or cy5.

In a preferred embodiment, the target sequence is labeled with, for example, a fluorescent, a chemiluminescent, a chemical, or a radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound target sequence. Unbound labeled streptavidin is typically removed prior to analysis.

As will be appreciated by those in the art, these assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions which allows formation of the label probe hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways. Components of the reaction may be added simultaneously, or sequentially, in different orders, with preferred embodiments outlined below. In addition, the reaction may include a variety of other reagents. These include salts, buffers, neutral proteins, e.g. albumin, detergents, etc. which may be used to facilitate optimal hybridization and detection, and/or reduce non-specific or background interactions. Reagents that otherwise improve the efficiency of the assay, such as protease inhibitors, nuclease inhibitors, anti-microbial agents, etc., may also be used as appropriate, depending on the sample preparation methods and purity of the target.

The assay data are analyzed to determine the expression levels, and changes in expression levels as between states, of individual genes, forming a gene expression profile.

Screens are performed to identify modulators of the angiogenesis phenotype. In one embodiment, screening is performed to identify modulators that can induce or suppress a particular expression profile, thus preferably generating the associated phenotype. In another embodiment, e.g., for diagnostic applications, having identified differentially expressed genes important in a particular state, screens can be performed to identify modulators that alter expression of individual genes. In an another embodiment, screening is performed to identify modulators that alter a biological function of the expression product of a differentially expressed gene. Again, having identified the importance of a gene in a particular state, screens are performed to identify agents that bind and/or modulate the biological activity of the gene product.

In addition screens can be done for genes that are induced in response to a candidate agent. After identifying a modulator based upon its ability to suppress an angiogenesis expression pattern leading to a normal expression pattern, or to modulate a single angiogenesis gene expression profile so as to mimic the expression of the gene from normal tissue, a screen as described above can be performed to identify genes that are specifically modulated in response to the agent. Comparing expression profiles between normal tissue and agent treated angiogenesis tissue reveals genes that are not expressed in normal tissue or angiogenesis tissue, but are expressed in agent treated tissue. These agent-specific sequences can be identified and used by methods described herein for angiogenesis genes or proteins. In particular these sequences and the proteins they encode find use in marking or identifying agent treated cells. In addition, antibodies can be raised against the agent induced proteins and used to target novel therapeutics to the treated angiogenesis tissue sample.

Thus, in one embodiment, a test compound is administered to a population of angiogenic cells, that have an associated angiogenesis expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e., a peptide) may be put into a viral construct such as an adenoviral or retroviral construct, and added to the cell, such that expression of the peptide agent is accomplished, e.g., PCT US97/01019. Regulatable gene therapy systems can also be used.

Once the test compound has been administered to the cells, the cells can be washed if desired and are allowed to incubate under preferably physiological conditions for some period of time. The cells are then harvested and a new gene expression profile is generated, as outlined herein.

Thus, for example, angiogenesis tissue may be screened for agents that modulate, e.g., induce or suppress the angiogenesis phenotype. A change in at least one gene, preferably many, of the expression profile indicates that the agent has an effect on angiogenesis activity. By defining such a signature for the angiogenesis phenotype, screens for new drugs that alter the phenotype can be devised. With this approach, the drug target need not be known and need not be represented in the original expression screening platform, nor does the level of transcript for the target protein need to change.

Measure of angiogenesis polypeptide activity, or of angiogenesis or the angiogenic phenotype can be performed using a variety of assays. For example, the effects of the test compounds upon the function of the anagiogenesis polypeptides can be measured by examining parameters described above. A suitable physiological change that affects activity can be used to assess the influence of a test compound on the polypeptides of this invention. When the functional consequences are determined using intact cells or animals, one can also measure a variety of effects such as, in the case of angiogenesis associated with tumors, tumor growth, neovascularization, hormone release, transcriptional changes to both known and uncharacterized genetic markers (e.g., northern blots), changes in cell metabolism such as cell growth or pH changes, and changes in intracellular second messengers such as cGMP. In the assays of the invention, mammalian angiogenesis polypeptide is typically used, e.g., mouse, preferably human.

A variety of angiogenesis assays are known to those of skill in the art. Various models have been employed to evaluate angiogenesis (e.g., Croix et al., Science 289:1197-1202, 2000 and Kahn et al., Amer. J. Pathol. 156:1887-1900). Assessement of angiogenesis in the presence of a potential modulator of angiogenesis can be performed using cell-cultre-based angiogenesis assays, e.g., endothelial cell tube formation assays, as well as other bioassays such as the chick CAM assay, the mouse corneal assay, and assays measuring the effect of administering potential modulators on implanted tumors. The chick CAM assay is described by O'Reilly, et al. Cell 79: 315-328, 1994. Briefly, 3 day old chicken embryos with intact yolks are separated from the egg and placed in a petri dish. After 3 days of incubation, a methylcellulose disc containing the protein to be tested is applied to the CAM of individual embryos. After about 48 hours of incubation, the embryos and CAMs are observed to determine whether endothelial growth has been inhibited. The mouse corneal assay involves implanting a growth factor-containing pellet, along with another pellet containing the suspected endothelial growth inhibitor, in the cornea of a mouse and observing the pattern of capillaries that are elaborated in the cornea. Angiogenesis can also be measured by determining the extent of neovascularization of a tumor. For example, carcinoma cells can be subcutaneously inoculated into athymic nude mice and tumor growth then monitored. The cancer cells are treated with an angiogenesis inhibitor, such as an antibody, or other compound that is exogenously administered, or can be transfected prior to inoculation with a polynucleotide inhibitor of angiogenesis. Immunoassays using endothelial cell-specific antibodies are typically used to stain for vascularization of tumor and the number of vessels in the tumor.

Assays to identify compounds with modulating activity can be performed in vitro. For example, an angiogenesis polypeptide is first contacted with a potential modulator and incubated for a suitable amount of time, e.g., from 0.5 to 48 hours. In one embodiment, the angiogenesis polypeptide levels are determined in vitro by measuring the level of protein or mRNA. The level of protein is measured using immunoassays such as western blotting, ELISA and the like with an antibody that selectively binds to the angiogenesis polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g.. fluorescently or radioactively labeled nucleic acids, radioactively or enzymatically labeled antibodies, and the like, as described herein.

Alternatively, a reporter gene system can be devised using the angiogenesis protein promoter operably linked to a reporter gene such as luciferase, green fluorescent protein, CAT, or β-gal. The reporter construct is typically transfected into a cell. After treatment with a potential modulator, the amount of reporter gene transcription, translation, or activity is measured according to standard techniques known to those of skill in the art.

In a preferred embodiment, as outlined above, screens may be done on individual genes and gene products (proteins). That is, having identified a particular differentially expressed gene as important in a particular state, screening of modulators of the expression of the gene or the gene product itself can be done. The gene products of differentially expressed genes are sometimes referred to herein as “angiogenesis proteins”. In preferred embodiments the angiogenesis protein comprises a sequence shown in Table 8. The angiogenesis protein may be a fragment, or alternatively, be the full length protein to a fragment shown herein.

Preferably, the angiogenesis protein is a fragment of approximately 14 to 24 amino acids long. More preferably the fragment is a soluble fragment. In one embodiment an angiogenesis protein is conjugated or fused to an immunogenic agent or BSA.

In one embodiment, screening for modulators of expression of specific genes is performed. Typically, the expression of only one or a few genes are evaluated. In another embodiment, screens are designed to first find compounds that bind to differentially expressed proteins. These compounds are then evaluated for the ability to modulate differentially expressed activity. Moreover, once initial candidate compounds are identified, variants can be further screened to better evaluate strucutre activity relationships.

In a preferred embodiment, binding assays are done. In general, purified or isolated gene product is used; that is, the gene products of one or more differentially expressed nucleic acids are made. For example, antibodies are generated to the protein gene products, and standard immunoassays are run to determine the amount of protein present. Alternatively, cells comprising the angiogenesis proteins can be used in the assays.

Thus, in a preferred embodiment, the methods comprise combining an angiogenesis protein and a candidate compound, and determining the binding of the compound to the angiogenesis protein. Preferred embodiments utilize the human angiogenesis protein, although other mammalian proteins may also be used, for example for the development of animal models of human disease. In some embodiments, as outlined herein, variant or derivative angiogenesis proteins may be used.

Generally, in a preferred embodiment of the methods herein, the angiogenesis protein or the candidate agent is non-diffusably bound to an insoluble support having isolated sample receiving areas (e.g. a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, teflon™, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the angiogenesis protein is bound to the support, and a test compound is added to the assay. Alternatively, the candidate agent is bound to the support and the angiogenesis protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the test modulating compound to the angiogenesis protein may be done in a number of ways. In a preferred embodiment, the compound is labelled, and binding determined directly, e.g., by attaching all or a portion of the angiogenesis protein to a solid support, adding a labelled candidate agent (e.g., a fluorescent label), washing off excess reagent, and determining whether the label is present on the solid support. Various blocking and washing steps may be utilized as appropriate.

By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin, etc. For the specific binding members, the complementary member would normally be labeled with a molecule which provides for detection, in accordance with known procedures, as outlined above. The label can directly or indirectly provide a detectable signal.

In some embodiments, only one of the components is labeled, e.g., the proteins (or proteinaceous candidate compounds) can be labeled. Alternatively, more than one component can be labeled with different labels, e.g., ¹²⁵I for the proteinsand a fluorophor for the compound. Proximity reagents, e.g., quenching or energy transfer reagents are also useful.

In one embodiment, the binding of the test compound is determined by competitive binding assay. The competitor is a binding moiety known to bind to the target molecule (i.e. an angiogenesis protein), such as an antibody, peptide, binding partner, ligand, etc. Under certain circumstances, there may be competitive binding between the compound and the binding moiety, with the binding moiety displacing the compound. In one embodiment, the test compound is labeled. Either the compound, or the competitor, or both, is added first to the protein for a time sufficient to allow binding, if present. Incubations may be performed at a temperature which facilitates optimal activity, typically between 4 and 40° C. Incubation periods are typically optimized, e.g., to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the test compound. Displacement of the competitor is an indication that the test compound is binding to the angiogenesis protein and thus is capable of binding to, and potentially modulating, the activity of the angiogenesis protein. In this embodiment, either component can be labeled. Thus, for example, if the competitor is labeled, the presence of label in the wash solution indicates displacement by the agent. Alternatively, if the test compound is labeled, the presence of the label on the support indicates displacement.

In an alternative embodiment, the test compound is added first, with incubation and washing, followed by the competitor. The absence of binding by the competitor may indicate that the test compound is bound to the angiogenesis protein with a higher affinity. Thus, if the test compound is labeled, the presence of the label on the support, coupled with a lack of competitor binding, may indicate that the test compound is capable of binding to the angiogenesis protein.

In a preferred embodiment, the methods comprise differential screening to identity agents that are capable of modulating the activitity of the angiogenesis proteins. In this embodiment, the methods comprise combining an angiogenesis protein and a competitor in a first sample. A second sample comprises a test compound, an angiogenesis protein, and a competitor. The binding of the competitor is determined for both samples, and a change, or difference in binding between the two samples indicates the presence of an agent capable of binding to the angiogenesis protein and potentially modulating its activity. That is, if the binding of the competitor is different in the second sample relative to the first sample, the agent is capable of binding to the angiogenesis protein.

Alternatively, differential screening is used to identify drug candidates that bind to the native angiogenesis protein, but cannot bind to modified angiogenesis proteins. The structure of the angiogenesis protein may be modeled, and used in rational drug design to synthesize agents that interact with that site. Drug candidates that affect the activity of an angiogenesis protein are also identified by screening drugs for the ability to either enhance or reduce the activity of the protein.

Positive controls and negative controls may be used in the assays. Preferably control and test samples are performed in at least triplicate to obtain statistically significant results. Incubation of all samples is for a time sufficient for the binding of the agent to the protein. Following incubation, samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc. which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in an order that provides for the requisite binding.

In a preferred embodiment, the invention provides methods for screening for a compound capable of modulating the activity of an angiogenesis protein. The methods comprise adding a test compound, as defined above, to a cell comprising angiogenesis proteins. Preferred cell types include almost any cell. The cells contain a recombinant nucleic acid that encodes an angiogenesis protein. In a preferred embodiment, a library of candidate agents are tested on a plurality of cells.

In one aspect, the assays are evaluated in the presence or absence or previous or subsequent exposure of physiological signals, for example hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, compounds that modulate angiogenesis agents are identified. Compounds with pharmacological activity are able to enhance or interfere with the activity of the angiogenesis protein. Once identified, similar structures are evaluated to identify critical structural feature of the compound.

In one embodiment, a method of inhibiting angiogenic cell division is provided. The method comprises administration of an angiogenesis inhibitor. In another embodiment, a method of inhibiting angiogenesis is provided. The method comprises administration of an angiogenesis inhibitor. In a further embodiment, methods of treating cells or individuals with angiogenesis are provided. The method comprises administration of an angiogenesis inhibitor.

In one embodiment, an angiogenesis inhibitor is an antibody as discussed above. In another embodiment, the angiogenesis inhibitor is an antisense molecule.

Polynucleotide Modulators of Angiogenesis

Antisense Polynucleotides

In certain embodiments, the activity of an angiogenesis-associated protein is downregulated, or entirely inhibited, by the use of antisense polynucleotide, i.e., a nucleic acid complementary to, and which can preferably hybridize specifically to, a coding mRNA nucleic acid sequence, e.g., an angiogenesis protein mRNA, or a subsequence thereof. Binding of the antisense polynucleotide to the mRNA reduces the translation and/or stability of the mRNA.

In the context of this invention, antisense polynucleotides can comprise naturally-occurring nucleotides, or synthetic species formed from naturally-occurring subunits or their close homologs. Antisense polynucleotides may also have altered sugar moieties or inter-sugar linkages. Exemplary among these are the phosphorothioate and other sulfur containing species which are known for use in the art. Analogs are comprehended by this invention so long as they function effectively to hybridize with the angiogenesis protein mRNA. See, e.g., Isis Pharmaceuticals, Carlsbad, Calif.; Sequitor, Inc., Natick, Mass.

Such antisense polynucleotides can readily be synthesized using recombinant means, or can be synthesized in vitro. Equipment for such synthesis is sold by several vendors, including Applied Biosystems. The preparation of other oligonucleotides such as phosphorothioates and alkylated derivatives is also well known to those of skill in the art.

Antisense molecules as used herein include antisense or sense oligonucleotides. Sense oligonucleotides can, e.g., be employed to block trancription by binding to the anti-sense strand. The antisense and sense oligonucleotide comprise a single-stranded nucleic acid sequence (either RNA or DNA) capable of binding to target mRNA (sense) or DNA (antisense) sequences for angiogenesis molecules. A preferred antisense molecule is for an angiogenesis sequences in Tables 1-8, or for a ligand or activator thereof. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

Ribozymes

In addition to antisense polynucleotides, ribozymes can be used to target and inhibit transcription of angiogenesis-associated nucleotide sequences. A ribozyme is an RNA molecule that catalytically cleaves other RNA molecules. Different kinds of ribozymes have been described, including group I ribozymes, hammerhead ribozymes, hairpin ribozymes, RNase P, and axhead ribozymes (see, e.g., Castanotto et al. (1994) Adv. in Pharmacology 25: 289-317 for a general review of the properties of different ribozymes).

The general features of hairpin ribozymes are described, e.g., in Hampel et al. (1990) Nucl. Acids Res. 18: 299-304; Hampel et al. (1990) European Patent Publication No. 0 360 257; U.S. Pat. No. 5,254,678. Methods of preparing are well known to those of skill in the art (see, e.g., Wong-Staal et al., WO 94/26877; Ojwang et al. (1993) Proc. Natl. Acad. Sci. USA 90: 6340-6344; Yamada et al. (1994) Human Gene Therapy 1: 39-45; Leavitt et al. (1995) Proc. Natl. Acad. Sci. USA 92: 699-703; Leavitt et al. (1994) Human Gene Therapy 5: 1151-120; and Yamada et al. (1994) Virology 205: 121-126).

Polynucleotide modulators of angiogenesis may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a polynucleotide modulator of angiogenesis may be introduced into a cell containing the target nucleic acid sequence, e.g., by formation of an polynucleotide-lipid complex, as described in WO 90/10448. It is understood that the use of antisense molecules or knock out and knock in models may also be used in screening assays as discussed above, in addition to methods of treatment.

Thus, in one embodiment, methods of modulating angiogenesis in cells or organisms are provided. In one embodiment, the methods comprise administering to a cell an anti-angiogenesis antibody that reduces or eliminates the biological activity of an endogeneous angiogenesis protein. Alternatively, the methods comprise administering to a cell or organism a recombinant nucleic acid encoding an angiogenesis protein. This may be accomplished in any number of ways. In a preferred embodiment, for example when the angiogenesis sequence is down-regulated in angiogenesis, such state may be reversed by increasing the amount of angiogenesis gene product in the cell. This can be accomplished, e.g., by overexpressing the endogeneous angiogenesis gene or administering a gene encoding the angiogenesis sequence, using known gene-therapy techniques, for example. In a preferred embodiment, the gene therapy techniques include the incorporation of the exogenous gene using enhanced homologous recombination (EHR), for example as described in PCT/US93/03868, hereby incorporated by reference in its entireity. Alternatively, for example when the angiogenesis sequence is up-regulated in angiogenesis, the activity of the endogeneous angiogenesis gene is decreased, for example by the administration of a angiogenesis antisense nucleic acid or other inhibitor, such as RNAi.

In one embodiment, the angiogenesis eproteins of the present invention may be used to generate polyclonal and monoclonal antibodies to angiogenesis proteins. Similarly, the angiogenesis proteins can be coupled, using standard technology, to affinity chromatography columns. These columns may then be used to purify angiogenesis antibodies useful for production, diagnostic, or therapeutic purposes. In a preferred embodiment, the antibodies are generated to epitopes unique to a angiogenesis protein; that is, the antibodies show little or no cross-reactivity to other proteins. The angiogenesis antibodies may be coupled to standard affinity chromatography columns and used to purify angiogenesis proteins. The antibodies may also be used as blocking polypeptides, as outlined above, since they will specifically bind to the angiogenesis protein.

Methods of Identifying Variant Angiogenesis-associated Sequences

Without being bound by theory, expression of various angiogenesis sequences is correlated with angiogenesis. Accordingly, disorders based on mutant or variant angiogenesis genes may be determined. In one embodiment, the invention provides methods for identifying cells containing variant angiogenesis genes, e.g., determining all or part of the sequence of at least one endogeneous angiogenesis genes in a cell. This may be accomplished using any number of sequencing techniques. In a preferred embodiment, the invention provides methods of identifying the angiogenesis genotype of an individual, e.g., determining all or part of the sequence of at least one angiogenesis gene of the individual. This is generally done in at least one tissue of the individual, and may include the evaluation of a number of tissues or different samples of the same tissue. The method may include comparing the sequence of the sequenced angiogenesis gene to a known angiogenesis gene, i.e., a wild-type gene.

The sequence of all or part of the angiogenesis gene can then be compared to the sequence of a known angiogenesis gene to determine if any differences exist. This can be done using any number of known homology programs, such as Bestfit, etc. In a preferred embodiment, the presence of a a difference in the sequence between the angiogenesis gene of the patient and the known angiogenesis gene correlates with a disease state or a propensity for a disease state, as outlined herein.

In a preferred embodiment, the angiogenesis genes are used as probes to determine the number of copies of the angiogenesis gene in the genome.

In another preferred embodiment, the angiogenesis genes are used as probes to determine the chromosomal localization of the angiogenesis genes. Information such as chromosomal localization finds use in providing a diagnosis or prognosis in particular when chromosomal abnormalities such as translocations, and the like are identified in the angiogenesis gene locus.

Administration of Pharmaceutical and Vaccine Compositions

In one embodiment, a therapeutically effective dose of an angiogenesis protein or modulator thereof, is administered to a patient. By “therapeutically effective dose” herein is meant a dose that produces effects for which it is administered. The exact dose will depend on the purpose of the treatment, and will be ascertainable by one skilled in the art using known techniques (e.g., Ansel et al., Pharmaceuitcal Dosage Forms and Drug Delivery, Lippincott, Williams & Wilkins Publishers, ISBN:0683305727; Lieberman (1992) Pharmaceutical Dosage Forms (vols. 1-3), Dekker, ISBN 0824770846, 082476918X, 0824712692, 0824716981; Lloyd (1999) The Art, Science and Technology of Pharmaceutical Compounding, Amer. Pharmacutical Assn, ISBN 0917330889; and Pickar (1999) Dosage Calculations, Delmar Pub, ISBN 0766805042). As is known in the art, adjustments for angiogenesis degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

The administration of the angiogenesis proteins and modulators thereof of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the angiogenesis proteins and modulators may be directly applied as a solution or spray.

The pharmaceutical compositions of the present invention comprise an angiogenesis protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that angiogenesis protein modulators (e.g., antibodies, antisense constructs, ribozymes, small organic molecules, etc.) when administered orally, should be protected from digestion. This is typically accomplished either by complexing the molecule(s) with a composition to render it resistant to acidic and enzymatic hydrolysis, or by packaging the molecule(s) in an appropriately resistant carrier, such as a liposome or a protection barrier. Means of protecting agents from digestion are well known in the art.

The compositions for administration will commonly comprise an angiogenesis protein modulator dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science, 15th ed., Mack Publishing Company, Easton, Pa. (1980) and Goodman and Gillman, The Pharmacologial Basis of Therapeutics, (Hardman, J. G, Limbird, L. E, Molinoff, P. B., Ruddon, R. W, and Gilman, A. G.,eds) The McGraw-Hill Companies, Inc., 1996).

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art, e.g., Remington's Pharmaceutical Science and Goodman and Gillman, The Pharmacologial Basis of Therapeutics, supra.

The compositions containing modulators of angiogenesis proteins can be administered for therapeutic or prophylactic treatments. In therapeutic applications, compositions are administered to a patient suffering from a disease (e.g., a cancer) in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the agents of this invention to effectively treat the patient. An amount of modulator that is capable of preventing or slowing the development of cancer in a mammal is referred to as a “prophylactically effective dose.” The particular dose required for a prophylactic treatment will depend upon the medical condition and history of the mammal, the particular cancer being prevented, as well as other factors such as age, weight, gender, administration route, efficiency, etc. Such prophylactic treatments may be used, e.g., in a mammal who has previously had cancer to prevent a recurrence of the cancer, or in a mammal who is suspected of having a significant likelihood of developing cancer.

It will be appreciated that the present angiogenesis protein-modulating compounds can be administered alone or in combination with additional angiogenesis modulating compounds or with other therapeutic agent, e.g., other anti-cancer agents or treatments.

In numerous embodiments, one or more nucleic acids, e.g., polynucleotides comprising nucleic acid sequences set forth in Tables 1-8, such as antisense polynucleotides or ribozymes, will be introduced into cells, in vitro or in vivo. The present invention provides methods, reagents, vectors, and cells useful for expression of angiogenesis-associated polypeptides and nucleic acids using in vitro (cell-free), ex vivo or in vivo (cell or organism-based) recombinant expression systems.

The particular procedure used to introduce the nucleic acids into a host cell for expression of a protein or nucleic acid is application specific. Many procedures for introducing foreign nucleotide sequences into host cells may be used. These include the use of calcium phosphate transfection, spheroplasts, electroporation, liposomes, microinjection, plasma vectors, viral vectors and any of the other well known methods for introducing cloned genomic DNA, cDNA, synthetic DNA or other foreign genetic material into a host cell (see, e.g., Berger and Kimmel, Guide to Molecular Cloning Techniques, Methods in Enzymology volume 152 Academic Press, Inc., San Diego, Calif. (Berger), F. M. Ausubel et al., eds., Current Protocols, a joint venture between Greene Publishing Associates, Inc. and John Wiley & Sons, Inc., (supplemented through 1999), and Sambrook et al., Molecular Cloning—A Laboratory Manual (2nd Ed.), Vol. 1-3, Cold Spring Harbor Laboratory, Cold Spring Harbor, N.Y., 1989.

In a preferred embodiment, angiogenesis proteins and modulators are administered as therapeutic agents, and can be formulated as outlined above. Similarly, angiogenesis genes (including both the full-length sequence, partial sequences, or regulatory sequences of the angiogenesis coding regions) can be administered in a gene therapy application. These angiogenesis genes can include antisense applications, either as gene therapy (i.e. for incorporation into the genome) or as antisense compositions, as will be appreciated by those in the art.

Angiogenesis polypeptides and polynucleotides can also be administered as vaccine compositions to stimulate HTL, CTL and antibody responses.. Such vaccine compositions can include, for example, lipidated peptides (e.g., Vitiello, A. et al., J. Clin. Invest. 95:341, 1995), peptide compositions encapsulated in poly(DL-lactide-co-glycolide) (“PLG”) microspheres (see, e.g., Eldridge, et al., Molec. Immunol. 28:287-294, 1991: Alonso et al., Vaccine 12:299-306, 1994; Jones et al., Vaccine 13:675-681, 1995), peptide compositions contained in immune stimulating complexes (ISCOMS) (see, e.g., Takahashi et al., Nature 344:873-875, 1990; Hu et al., Clin Exp Immunol. 113:235-243, 1998), multiple antigen peptide systems (MAPs) (see e.g., Tam, J. P., Proc. Natl. Acad. Sci. U.S.A. 85:5409-5413, 1988; Tam, J. P., J. Immunol. Methods 196:17-32, 1996), peptides formulated as multivalent peptides; peptides for use in ballistic delivery systems, typically crystallized peptides, viral delivery vectors (Perkus, M. E. et al., In: Concepts in vaccine development, Kaufmann, S. H. E., ed., p. 379, 1996; Chakrabarti, S. et al., Nature 320:535, 1986; Hu, S. L. et al., Nature 320:537, 1986; Kieny, M.-P. et al., AIDS Bio/Technology 4:790, 1986; Top, F. H. et al., J. Infect. Dis. 124:148, 1971; Chanda, P. K. et al., Virology 175:535, 1990), particles of viral or synthetic origin (e.g., Kofler, N. et al., J. Immunol. Methods. 192:25, 1996; Eldridge, J. H. et al., Sem. Hematol. 30:16, 1993; Falo, L. D., Jr. et al., Nature Med. 7:649, 1995), adjuvants (Warren, H. S., Vogel, F. R., and Chedid, L. A. Annu. Rev. Immunol. 4:369, 1986; Gupta, R. K. et al., Vaccine 11:293, 1993), liposomes (Reddy, R. et al., J. Immunol. 148:1585, 1992; Rock, K. L., Immunol. Today 17:131, 1996), or, naked or particle absorbed cDNA (Ulmer, J. B. et al., Science 259:1745, 1993; Robinson, H. L., Hunt, L. A., and Webster, R. G., Vaccine 11:957, 1993; Shiver, J. W. et al., In: Concepts in vaccine development, Kaufmann, S. H. E., ed., p. 423, 1996; Cease, K. B., and Berzofsky, J. A., Annu. Rev. Immunol. 12:923, 1994 and Eldridge, J. H. et al., Sem. Hematol. 30:16, 1993). Toxin-targeted delivery technologies, also known as receptor mediated targeting, such as those of Avant Immunotherapeutics, Inc. (Needham, Mass.) may also be used.

Vaccine compositions often include adjuvants. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Vaccines can be administered as nucleic acid compositions wherein DNA or RNA encoding one or more of the polypeptides, or a fragment thereof, is administered to a patient. This approach is described, for instance, in Wolff et. al., Science 247:1465 (1990) as well as U.S. Pat. Nos. 5,580,859; 5,589,466; 5,804,566; 5,739,118; 5,736,524; 5,679,647; WO 98/04720; and in more detail below. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

For therapeutic or prophylactic immunization purposes, the peptides of the invention can be expressed by viral or bacterial vectors. Examples of expression vectors include attenuated viral hosts, such as vaccinia or fowlpox. This approach involves the use of vaccinia virus, for example, as a vector to express nucleotide sequences that encode angiogenic polypeptides or polypeptide fragments. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover et al., Nature 351:456-460 (1991). A wide variety of other vectors useful for therapeutic administration or immunization e.g. adeno and adeno-associated virus vectors, retroviral vectors, Salmonella typhi vectors, detoxified anthrax toxin vectors, and the like, will be apparent to those skilled in the art from the description herein (see, e.g., Shata et al. (2000) Mol Med Today, 6: 66-71; Shedlock et al., J Leukoc Biol 68,:793-806, 2000; Hipp et al., In Vivo 14:571-85, 2000).

Methods for the use of genes as DNA vaccines are well known, and include placing an angiogenesis gene or portion of an angiogenesis gene under the control of a regulatable promoter or a tissue-specific promoter for expression in an angiogenesis patient. The angiogenesis gene used for DNA vaccines can encode full-length angiogenesis proteins, but more preferably encodes portions of the angiogenesis proteins including peptides derived from the angiogenesis protein. In one embodiment, a patient is immunized with a DNA vaccine comprising a plurality of nucleotide sequences derived from an angiogenesis gene. For example, angiogenesis-associated genes or sequence encoding subfragments of an angiogenesis protein are introduced into expression vectors and tested for their immunogenicity in the context of Class I MHC and an ability to generate cytotoxic T cell responses. This procedure provides for production of cytotoxic T cell responses against cells which present antigen, including intracellular epitopes.

In a preferred embodiment, the DNA vaccines include a gene encoding an adjuvant molecule with the DNA vaccine. Such adjuvant molecules include cytokines that increase the immunogenic response to the angiogenesis polypeptide encoded by the DNA vaccine. Additional or alternative adjuvants are available.

In another preferred embodiment angiogenesis genes find use in generating animal models of angiogenesis. When the angiogenesis gene identified is repressed or diminished in angiogenesic tissue, gene therapy technology, e.g., wherein antisense RNA directed to the angiogenesis gene will also diminish or repress expression of the gene. Animal models of angiogenesis find use in screening for modulators of an angiogenesis-associated sequence or modulators of angiogenesis. Similarly, transgenic animal technology including gene knockout technology, for example as a result of homologous recombination with an appropriate gene targeting vector, will result in the absence or increased expression of the angiogenesis protein. When desired, tissue-specific expression or knockout of the angiogenesis protein may be necessary.

It is also possible that the angiogenesis protein is overexpressed in angiogenesis. As such, transgenic animals can be generated that overexpress the angiogenesis protein. Depending on the desired expression level, promoters of various strengths can be employed to express the transgene. Also, the number of copies of the integrated transgene can be determined and compared for a determination of the expression level of the transgene. Animals generated by such methods find use as animal models of angiogenesis and are additionally useful in screening for modulators to treat angiogenesis or to evaluate a therapeutic entity.

Kits for Use in Diagnostic and/or Prognostic Applications

For use in diagnostic, research, and therapeutic applications suggested above, kits are also provided by the invention. In the diagnostic and research applications such kits may include any or all of the following: assay reagents, buffers, angiogenesis-specific nucleic acids or antibodies, hybridization probes and/or primers, antisense polynucleotides, ribozymes, dominant negative angiogenesis polypeptides or polynucleotides, small molecules inhibitors of angiogenesis-associated sequences etc. A therapeutic product may include sterile saline or another pharmaceutically acceptable emulsion and suspension base.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The present invention also provides for kits for screening for modulators of angiogenesis-associated sequences. Such kits can be prepared from readily available materials and reagents. For example, such kits can comprise one or more of the following materials: an angiogenesis-associated polypeptide or polynucleotide, reaction tubes, and instructions for testing angiogenic-associated activity. Optionally, the kit contains biologically active angiogenesis protein. A wide variety of kits and components can be prepared according to the present invention, depending upon the intended user of the kit and the particular needs of the user. Diagnosis would typically involve evaluation of a plurality of genes or products. The genes will be selected based on correlations with important parameters in disease which may be identified in historical or outcome data.

It is understood that the examples described above in no way serve to limit the true scope of this invention, but rather are presented for illustrative purposes. All publications, sequences of accession numbers, and patent applications cited in this specification are herein incorporated by reference as if each individual publication or patent application were specifically and individually indicated to be incorporated by reference.

### EXAMPLES

### Example 1

### Tissue Preparation, Labeling Chips, and Fingerprints

Purify Total RNA from Tissue Using TRIzol Reagent

Homogenize tissue samples in 1 ml of TRIzol per 50 mg of tissue using a Polytron 3100 homogenizer. The generator/probe used depends upon the tissue size. A generator that is too large for the amount of tissue to be homogenized will cause a loss of sample and lower RNA yield. TRIzol is added directly to frozen tissue, which is then homogenize. Following homogenization, insoluble material is removed by centrifugation at 7500×g for 15 min in a Sorvall superspeed or 12,000×g for 10 min. in an Eppendorf centrifuge at 4° C. The clear homogenate is transferred to a new tube for use. The samples may be frozen now at −60° to −70° C. (and kept for at least one month). The homogenate is mixed with 0.2 ml of chloroform per 1 ml of TRIzol reagent used in the original homogenization and incubated at room temp. for 2-3 minutes. The aqueous phase is then separated by centrifugation and transferred to a fresh tube and the RNA precipitated using isopropyl alcohol. The pellet is isolated by centrifugation, washed, air-dried, resuspended in an appropriate volume of DEPC H₂O, and the absorbance measured.

Purification of poly A+ mRNA from total RNA is performed as follows. Heat an oligotex suspension to 37° C. and mixing immediately before adding to RNA. The Elution Buffer is heated at 70° C. Warm up 2×Binding Buffer at 65° C. if there is precipitate in the buffer. Mix total RNA with DEPC-treated water, 2×Binding Buffer, and Oligotex according to Table 2 on page 16 of the Oligotex Handbook. Incubate for 3 minutes at 65° C. Incubate for 10 minutes at room temperature. Centrifuge for 2 minutes at 14,000 to 18,000 g. Remove supernatant without disturbing Oligotex pellet. A little bit of solution can be left behind to reduce the loss of Oligotex. Gently resuspend in Wash Buffer OW2 and pipet onto spin column. Centrifuge the spin column at full speed for 1 minute. Transfer spin column to a new collection tube and gently resuspend in Wash Buffer OW2 and centrifuge as describe herein. Transfer spin column to a new tube and elute with 20 to 100 ul of preheated (70° C.) Elution Buffer. Gently resuspend Oligotex resin by pipetting up and down. Centrifuge as above. Repeat elution with fresh elution buffer or use first eluate to keep the elution volume low. Read absorbance, using diluted Elution Buffer as the blank. Before proceeding with cDNA synthesis, precipitate the mRNA as follows: add 0.4 vol. of 7.5 M NH4OAc+2.5 vol. of cold 100% ethanol. Precipitate at −20° C. 1 hour to overnight (or 20-30 min. at −70° C). Centrifuge at 14,000-16,000×g for 30 minutes at 4° C. Wash pellet with 0.5 ml of 80% ethanol (−20° C.) then centrifuge at 14,000-16,000×g for 5 minutes at room temperature. Repeat 80% ethanol wash. Air dry the ethanol from the pellet in the hood.. Suspend pellet in DEPC H₂O at 1 ug/ul concentration.

To further Clean up total RNA using Qiagen's RNeasy kit, add no more than 100 ug to an RNeasy column. Adjust sample to a volume of 100 ul with RNase-free water. Add 350 ul Buffer RLT then 250 ul ethanol (100%) to the sample. Mix by pipetting (do not centrifuge) then apply sample to an RNeasy mini spin column. Centrifuge for 15 sec at >10,000 rpm. Transfer column to a new 2-ml collection tube. Add 500 ul Buffer RPE and centrifuge for 15 sec at >10,000 rpm. Discard flowthrough. Add 500 ul Buffer RPE and centrifuge for 15 sec at >10,000 rpm. Discard flowthrough then centrifuge for 2 min at maximum speed to dry column membrane. Transfer column to a new 1.5-ml collection tube and apply 30-50 ul of RNase-free water directly onto column membrane. Centrifuge 1 min at >10,000 rpm. Repeat elution. and read absorbance.

cDNA Synthesis Using Gibco's “SuperScript Choice System for cDNA Synthesis” Kit

First Strand cDNA synthesis is performed as follows. Use 5 ug of total RNA or 1 ug of polyA+ mRNA as starting material. For total RNA, use 2 ul of SuperScript RT. For polyA+ mRNA, use 1 ul of SuperScript RT. Final volume of first strand synthesis mix is 20 ul. RNA must be in a volume no greater than 10 ul. Incubate RNA with 1 ul of 100 pmol T7-T24 oligo for 10 min at 70 C. On ice, add 7 ul of: 4 ul 5×1st Strand Buffer, 2 ul of 0.1M DTT, and 1 ul of 10 mM dNTP mix. Incubate at 37 C. for 2 min then add SuperScript RT. Incubate at 37 C. for 1 hour.

For the second strand synthesis, place 1st strand reactions on ice and add: 91 ul DEPC H₂O; 30 ul 5×2nd Strand Buffer; 3 ul 100 mM dNTP mix; 1 ul 10 U/ul E.coli DNA Ligase; 4 ul 10 U/ul E.coli DNA Polymerase; and 1 ul 2 U/ul RNase H. Mix and incubate 2 hours at 16 C. Add 2 ul T4 DNA Polymerase. Incubate 5 min at 16 C. Add 10 ul of 0.5M EDTA. A further clean-up of DNA is performed using phenol:chloroform:isoamyl Alcohol (25:24:1) purification.

In vitro Transcription (IVT) and labeling with biotin is performed as follows: Pipet 1.5 ul of cDNA into a thin-wall PCR tube. Make NTP labeling mix by combining 2 ul T7 10×ATP (75 mM) (Ambion); 2 ul T7 10×GTP (75 mM) (Ambion); 1.5 ul T7 10×CTP (75 mM) (Ambion); 1.5 ul T7 10×UTP (75 mM) (Ambion); 3.75 ul 10 mM Bio-11-UTP (Boehringer-Mannheim/Roche or Enzo); 3.75 ul 10 mM Bio-16-CTP (Enzo); 2 ul 10×T7 transcription buffer (Ambion); and 2 ul 10×T7 enzyme mix (Ambion). The final volume is 20 ul. Incubate 6 hours at 37° C. in a PCR machine. The RNA can be furthered cleaned.

Fragmentation is performed as follows. 15 ug of labeled RNA is usually fragmented. Try to minimize the fragmentation reaction volume; a 10 ul volume is recommended but 20 ul is all right. Do not go higher than 20 ul because the magnesium in the fragmentation buffer contributes to precipitation in the hybridization buffer. Fragment RNA by incubation at 94 C. for 35 minutes in 1×Fragmentation buffer (5×Fragmentation buffer is 200 mM Tris-acetate, pH 8.1; 500 mM KOAc; 150 mM MgOAc). The labeled RNA transcript can be analyzed before and after fragmentation. Samples can be heated to 65° C. for 15 minutes and electrophoresed on 1% agarose/TBE gels to get an approximate idea of the transcript size range

For hybridization, 200 ul (10 ug cRNA) of a hybridization mix is put on the chip. If multiple hybridizations are to be done (such as cycling through a 5 chip set), then it is recommended that an initial hybridization mix of 300 ul or more be made. The hybridization mix is: fragment labeled RNA (50 ng/ul final conc.); 50 pM 948-b control oligo; 1.5 pM BioB; 5 pM BioC; 25 pM BioD; 100 pM CRE; 0.1 mg/ml herring sperm DNA; 0.5 mg/ml acetylated BSA; and 300 ul with 1×MES hyb buffer.

Labeling is performed as follows: The hybridization reaction includes non-biotinylated IVT (purified by RNeasy columns); IVT antisense RNA 4 μg:μl; random Hexamers (1 μg/μl) 4 μl and water to 14 ul. The reaciton is incubated at 70° C., 10 min. Reverse transcriptionis performed in the following reaction: 5×First Strand (BRL) buffer, 6 μl; 0.1 M DTT, 3 μl; 50×dNTP mix, 0.6 μl; H₂O, 2.4 μl; Cy3 or Cy5 dUTP (1 mM), 3 μl; SS RT II (BRL), 1 μl in a final volume of 16 μl. Add to hybridization reaction. Incubate 30 min., 42° C. Add 1 μl SSII and incubate another hour. Put on ice. 50×dNTP mix (25 mM of cold dATP, dCTP, and dGTP, 10 mM of dTTP: 25 μl each of 100 mM dATP, dCTP, and dGTP; 10 μl of 100 mM dTTP to 15 μl H2O. dNTPs from Pharmacia)

RNA degradation is performed as follows. Add 86 μl H2O, 1.5 μl 1M NaOH/2 mM EDTA and incubate at 65° C., 10 min.. For U-Con 30, 500 μl TE/sample spin at 7000 g for 10 min, save flow through for purification. For Qiagen purification, suspend u-con recovered material in 500 μl buffer PB and proceed using Qiagen protocol. For DNAse digestion, add 1 ul of 1/100 dil of DNAse/30 ul Rx and incubate at 37° C. for 15 min. Incubate at 5 min 95° C. to denature the DNAse/

For sample preparation, add Cot-1 DNA, 10 μl; 50×dNTPs, 1 μl; 20×SSC, 2.3 μl; Na pyro phosphate, 7.5 μl; 10 mg/ml Herring sperm DNA; 1 ul of 1/10 dilution to 21.8 final vol. Dry in speed vac. Resuspend in 15 μl H2O. Add 0.38 μl 10% SDS. Heat 95° C., 2 min and slow cool at room temp. for 20 min. Put on slide and hybridize overnight at 64° C. Washing after the hybridization: 3×SSC/0.03% SDS: 2 min., 37.5 mls 20×SSC+0.75 mls 10% SDS in 250 mls H2O; 1×SSC: 5 min., 12.5 mls 20×SSC in 250 mls H2O; 0.2×SSC: 5 min., 2.5 mls 20×SSC in 250 mls H2O. Dry slides and scan at appropiate PMT's and channels.

### Example 2

### A Model of Angiogenesis is Used to Determine Expression in Angiogenesis

In the model of angiogenesis used to determine expression of angiogenesis-associated sequences, human umbilical vein endothelial cells (HUVEC) were obtained, e.g., as passage 1 (p1) frozen cells from Cascade Biologics (Oregon) and grown in maintenance medium: Medium 199 (Life Technologies) supplemented with 20% pooled human serum, 100 mg/ml heparin and 75 mg/ml endothelial cell growth supplements (Sigma) and gentamicin (Life Technologies). An in vitro cell system model was used in which 2×10⁵ HUVECs were cultured in 0.5 ml 3 mgs/ml plasminogen-depleted fibrinogen (Calbiochem, San Diego, Calif.) that was polymerized by the addition of 1 unit of maintenance medium supplemented with 100 ng/ml VEGF and HGF and 10 ng/ml TGF-α (R&D Systems, Minneapolis, Minn.) added (growth medium). The growth medium was replaced every 2 days. Samples for RNA were collected, e.g., at 0, 2, 6, 15, 24, 48, and 96 hours of culture. The fibrin clots were placed in Trizol (Life Technologies) and disrupted using a Tissuemizer. Thereafter standard procedures were used for extracting the RNA (e.g., Example 1).

Angiogenesis associated sequences thus identified are shown in Tables 1-8. As indicated, some of the Accession numbers include expression sequence tags (ESTs). Thus, in one embodiment herein, genes within an expression profile, also termed expression profile genes, include ESTs and are not necessarily full length.

TABLE 1

| | |
|---|---|
| Pkey: | Unique Eos probeset identifier number |
| Accession: | Accession number used for previous patent filings |
| ExAccn: | Exemplar Accession number, Genbank accession number |
| UnigeneID: | Unigene number |
| Unigene Title: | Unigene gene title |

| Pkey | Accession | ExAccn | UnigeneID | UnigeneTitle |
|---|---|---|---|---|
| 134404 | AB000450 | AB000450 | Hs.82771 | vaccinia related kinase 2 |
| 121443 | AB002380 | AF180681 | Hs.6582 | Rho guanine exchange factor (GEF) 12 |
| 100082 | AB003103 | AA130080 | Hs.4295 | proteasome (prosome, macropain) 26S subunit, non-ATPase, 12 |
| 132817 | AB004884 | N27852 | Hs 57553 | tousled-like kinase 2 |
| 130150 | AF000573_rna1 | BE094848 | Hs.15113 | homogentisate 1,2-dioxygenase (homogentisate oxidase) |
| 100104 | AF008937 | AF008937 | Hs.102178 | syntaxin 16 |
| 130839 | AF009301 | AB011169 | Hs.20141 | similar to S. cerevisiae SSM4 |
| 427064 | AF009368 | AF029674 | Hs 173422 | KIAA1605 protein |
| 100113 | D00591 | NM_001269 | Hs 84746 | chromosome condensation 1 |
| 133980 | D00760 | AA294921 | Hs.250811 | v-ral simian leukemia viral oncogene homolog B (ras related; GTP |
| | | | | binding protein) |
| 100129 | D11139 | AA469369 | Hs 5831 | tissue inhibitor of metalloproteinase 1 (erythroid potentiating |
| | | | | activity, collagenase inhibitor) |
| 100154 | D14657 | H60720 | Hs 81892 | KIAA0101 gene product |
| 100169 | D14878 | AL037228 | Hs.82043 | D123 gene product |
| 101956 | D17716 | NM_002410 | Hs.121502 | mannosyl (alpha-1,6-)-glycoprotein beta-1,6-N-acetyl- |
| | | | | glucosaminyltransferase |
| 100190 | D21090 | M91401 | Hs.178658 | RAD23 (S. cerevisiae) homolog B |
| 134742 | D26135 | NM_001346 | Hs.89462 | diacylglycerol kinase, gamma (90 kD) |
| 100211 | D26528 | D26528 | Hs.123058 | DEAD/H (Asp-Glu-Ala-Asp/His) box polypeptide 7 (RNA helicase, 52 |
| | | | | kD) |
| 100238 | D30742 | L24959 | Hs.348 | calcium/calmodulin-dependent protein kinase IV |
| 130283 | D31762 | NM_012288 | Hs.153954 | TRAM-like protein |
| 134237 | D31765 | D31765 | Hs.170114 | KIAA0061 protein |
| 100248 | D31888 | NM_015156 | Hs 78398 | KIAA0071 protein |
| 100256 | D38128 | D25418 | Hs 393 | prostaglandin 12 (prostacyclin) receptor (IP) |
| 100262 | D38500 | D38500 | Hs.278468 | postmeiotic segregation increased 2-like 4 |
| 134329 | D38551 | N92036 | Hs 81848 | RAD21 (S. pombe) homolog |
| 100281 | D42087 | AF091035 | Hs 184627 | KIAA0118 protein |
| 100294 | D49396 | AA331881 | Hs 75454 | peroxiredoxin 3 |
| 100327 | D55640 | D55640 | | gb: Human monocyte PABL (pseudoautosomal boundary-like sequence) |
| | | | | mRNA, clone Mo2. |
| 100335 | D63391 | AW247529 | Hs 6793 | platelet-activating factor acetylhydrolase, isoform Ib, gamma |
| | | | | subunit (29 kD) |
| 134495 | D63477 | D63477 | Hs.84087 | KIAA0143 protein |
| 100338 | D63483 | D86864 | Hs.57735 | acetyl LDL receptor; SREC |
| 135152 | D64015 | M96954 | Hs 182741 | TIA1 cytotoxic granule-associated RNA-binding protein-like 1 |
| 134269 | D79990 | NM_014737 | Hs.80905 | Ras association (RaIGDS/AF-6) domain family 2 |
| 100372 | D79997 | NM_014791 | Hs.184339 | KIAA0175 gene product |
| 134304 | D80010 | BE613486 | Hs 81412 | lipin 1 |
| 100394 | D84276 | D84284 | Hs.66052 | CD38 antigen (p45) |
| 100405 | D86425 | AW291587 | Hs 82733 | nidogen 2 |
| 100418 | D86978 | D86978 | Hs.84790 | KIAA0225 protein |
| 133154 | D87012 | D87012 | Hs 194685 | topoisomerase (DNA) III beta |
| 134347 | D87075 | AF164142 | Hs.82042 | solute carrier family 23 (nucleobase transporters), member 1 |
| 128653 | D87432 | D87432 | Hs.10315 | solute carrier family 7 (cationic amino acid transporter, y+ |
| | | | | system), member 6 |
| 100438 | D87448 | AA013051 | Hs.91417 | topoisomerase (DNA) II binding protein |
| 134593 | D87845 | NM_000437 | Hs.234392 | platelet-activating factor acetylhydrolase 2 (40 kD) |
| 100481 | HG1098-HT1098 | X70377 | Hs.121489 | cystatin D |
| 100552 | HG2167-HT2237 | AA019521 | Hs.301946 | lysosomal |
| 100591 | HG2415-HT2511 | NM_004091 | Hs.231444 | Homo sapiens, Similar to hypothetical protein PRO1722, clone |
| | | | | MGC: 15692, mRNA, complete cds |
| 100652 | HG2825-HT2949 | BE613608 | Hs.142653 | ret finger protein |
| 100662 | HG2887-HT3031_r | AI368680 | Hs 816 | SRY (sex determining region Y)-box 2 |
| 100899 | HG4660-HT5073 | AL039123 | Hs.103042 | microtubule-associated protein 1B |
| 100905 | HG4704-HT5146 | L12260 | Hs.172816 | neuregulin 1 |
| 100945 | HG884-HT884 | AF002225 | Hs.180686 | ubiquitin protein ligase E3A (human papilloma virus E6-associated |
| | | | | protein, Angelman syndrome) |
| 100950 | HG919-HT919 | AF128542 | Hs.166846 | polymerase (DNA directed), epsilon |
| 100964 | J00212_f | J00212 | | Empirically selected from AFFX single probeset |
| 135407 | J04029 | J04029 | Hs.99936 | keratin 10 (epidermolytic hyperkeratosis; keratosis palmaris et |
| | | | | plantaris) |
| 130149 | J04031 | AW067805 | Hs.172665 | methylenetetrahydrofolate dehydrogenase (NADP + dependent), |
| | | | | methenyltetrahydrofolate |
| 131877 | J04088 | J04088 | Hs.156346 | topoisomerase (DNA) II alpha (170 kD) |
| 101016 | J04543 | J04543 | Hs.78637 | annexin A7 |
| 134786 | L06139 | T29618 | Hs.89640 | TEK tyrosine kinase, endothelial (venous malformations, multiple |
| | | | | cutaneous and mucosal) |
| 134100 | L07540 | AA460085 | Hs.171075 | replication factor C (activator 1) 5 (36.5 kD) |
| 134078 | L08895 | L08895 | Hs.78995 | MADS box transcription enhancer factor 2, polypeptide C (myocyte |
| | | | | enhancer factor 2C) |
| 101132 | L11239 | L11239 | Hs 36993 | gastrulation brain homeo box 1 |
| 134849 | L11353 | BE409525 | Hs.902 | neurofibromin 2 (bilateral acoustic neuroma) |
| 106432 | L13773 | AK000310 | Hs.17138 | hypothetical protein FLJ20303 |
| 101152 | L13800 | AI984625 | Hs 9884 | spindle pole body protein |
| 135397 | L14922 | L14922 | Hs.166563 | replication factor C (activator 1) 1 (145 kD) |
| 131687 | L15189 | BE297635 | Hs.3069 | heat shock 70 kD protein 9B (mortalin-2) |
| 101168 | L15388 | NM_005308 | Hs 211569 | G protein-coupled receptor kinase 5 |
| 421155 | L16895 | H87879 | Hs.102267 | lysyl oxidase |
| 101226 | L27476 | AF083892 | Hs.75608 | tight junction protein 2 (zona occludens 2) |
| 133975 | L27624 | C18356 | Hs.295944 | tissue factor pathway inhibitor 2 |
| 134739 | L32976 | NM_002419 | Hs.89449 | mitogen-activated protein kinase kinase kinase 11 |
| 130155 | L33404 | AA101043 | Hs.151254 | kallikrein 7 (chymotryptic, stratum corneum) |
| 440538 | L35263 | W76332 | Hs.79107 | mitogen-activated protein kinase 14 |
| 132813 | L37347 | BE313625 | Hs.57435 | solute carrier family 11 (proton-coupled divalent metal ion |
| | | | | transporters), member 2 |
| 101294 | L40371 | AF168418 | Hs.116784 | thyroid hormone receptor interactor 4 |
| 101300 | L40391 | BE535511 | Hs.74137 | transmembrane trafficking protein |
| 101310 | L41607 | L41607 | Hs.934 | glucosaminyl (N-acetyl) transferase 2, I-branching enzyme |
| 130344 | L77566 | AW250122 | Hs.154879 | DiGeorge syndrome critical region gene DGSI; likely ortholog of |
| | | | | mouse expressed sequence 2 embryonic lethal |
| 101381 | M13928 | AW675039 | Hs.1227 | aminolevulinate, delta-, dehydratase |
| 101668 | M14016 | AW005903 | Hs.78601 | uroporphyrinogen decarboxylase |
| 133780 | M14219 | AA557660 | Hs.76152 | decorin |
| 101396 | M15796 | BE267931 | Hs.78996 | proliferating cell nuclear antigen |
| 101447 | M21305 | M21305 | | gb: Human alpha satellite and satellite 3 junction DNA sequence. |
| 101458 | M22092 | M22092 | | gb: Human neural cell adhesion molecule (N-CAM) gene, exon SEC and |
| | | | | partial cds. |
| 101470 | M22898 | NM_000546 | Hs 1846 | tumor protein p53 (Li-Fraumeni syndrome) |
| 134604 | M22995 | NM_002884 | Hs.865 | RAP1A, member of RAS oncogene family |
| 101478 | M23379 | NM_002890 | Hs.758 | RAS p21 protein activator (GTPase activating protein) 1 |
| 406698 | M24364 | X03068 | Hs.73931 | major histocompatibility complex, class II, DQ beta 1 |
| 133519 | M24400 | AW583062 | Hs.74502 | chymotrypsinogen B1 |
| 131185 | M25753 | BE280074 | Hs.23960 | cyclin B1 |
| 134116 | M27691 | R84694 | Hs.79194 | cAMP responsive element binding protein 1 |
| 133999 | M28213 | AA535244 | Hs 78305 | RAB2, member RAS oncogene family |
| 130174 | M29550 | M29551 | Hs.151531 | protein phosphatase 3 (formerly 2B), catalytic subunit, beta |
| | | | | isoform (calcineurin A beta) |
| 129963 | M29971 | M29971 | Hs.1384 | O-6-methylguanine-DNA methyltransferase |
| 132983 | M30269 | M30269 | Hs.62041 | nidogen (enactin) |
| 133900 | M31158 | M31158 | Hs.77439 | protein kinase, cAMP-dependent, regulatory, type II, beta |
| 101543 | M31166 | M31166 | Hs.2050 | pentaxin-related gene, rapidly induced by IL-1 beta |
| 101545 | M31210 | BE246154 | Hs.154210 | endothelial differentiation, sphingolipid G-protein-coupled |
| | | | | receptor, 1 |
| 101620 | M55420 | S55271 | Hs 247930 | Epsilon, IgE |
| 134691 | M59979 | AW382987 | Hs.88474 | prostaglandin-endoperoxide synthase 1 (prostaglandin G/H |
| | | | | synthase and cyclooxygenase) |
| 133595 | M62810 | AA393273 | Hs.75133 | transcription factor 6-like 1 (mitochondrial transcription factor |
| | | | | 1-like) |
| 130425 | M63838 | AA243383 | Hs.155530 | interferon, gamma-inducible protein 16 |
| 101700 | M64710 | D90337 | Hs.247916 | natriuretic peptide precursor C |
| 101714 | M68874 | M68874 | Hs.211587 | phospholipase A2, group IVA (cytosolic, calcium-dependent) |
| 134246 | M74524 | D28459 | Hs.80612 | ubiquitin-conjugating enzyme E2A (RAD6 homolog) |
| 101760 | M80254 | M80254 | Hs.173125 | peptidylprolyl isomerase F (cyclophilin F) |
| 133948 | M81780_cds3 | X59960 | Hs.77813 | sphingomyelin phosphodiesterase 1, acid lysosomal (acid |
| | | | | sphingomyelinase) |
| 101791 | M83822 | M83822 | Hs 62354 | cell division cycle 4-like |
| 101812 | M86934 | BE439894 | Hs.78991 | DNA segment, numerous copies, expressed probes (GS1 gene) |
| 101813 | M87338 | NM_002914 | Hs.139226 | replication factor C (activator 1) 2 (40 kD) |
| 133396 | M96326_rna1 | M96326 | Hs 72885 | azurocidin 1 (cationic antimicrobial protein 37) |
| 135152 | M96954 | M96954 | Hs.182741 | TIA1 cytotoxic granule-associated RNA-binding protein-like 1 |
| 129026 | M98833 | AL120297 | Hs.108043 | Friend leukemia virus integration 1 |
| 101901 | S66793 | H38026 | Hs.308 | arrestin 3, retinal (X-arrestin) |
| 134831 | S72370 | AA853479 | Hs.89890 | pyruvate carboxylase |
| 134039 | S78569 | NM_002290 | Hs.78672 | laminin, alpha 4 |
| 134395 | S79873 | AA456539 | Hs 8262 | lysosomal |
| 101975 | S83325 | AA079717 | Hs.283664 | aspartate beta-hydroxylase |
| 101977 | S83364 | AF112213 | Hs 184062 | putative Rab5-interacting protein |
| 101978 | S83365 | BE561610 | Hs.5809 | putative transmembrane protein; homolog of yeast Golgi membrane |
| | | | | protein Yif1p (Yip1p-interacting factor) |
| 101998 | U01212 | U01212 | Hs.248153 | olfactory marker protein |
| 102003 | U01922 | U01922 | Hs.125565 | translocase of inner mitochondrial membrane 8 (yeast) homolog A |
| 102007 | U02556 | U02556 | Hs 75307 | t-complex-associated-testis-expressed 1-like |
| 102009 | U02680 | BE245149 | Hs 82643 | protein tyrosine kinase 9 |
| 416658 | U03272 | U03272 | Hs.79432 | fibrillin 2 (congenital contractural arachnodactyly) |
| 132951 | U04209 | AW821182 | Hs.61418 | microfibrillar-associated protein 1 |
| 135389 | U05237 | U05237 | Hs.99872 | fetal Alzheimer antigen |
| 102048 | U07225 | U07225 | Hs 339 | purinergic receptor P2Y, G-protein coupled, 2 |
| 130145 | U07620 | U34820 | Hs.151051 | mitogen-activated protein kinase 10 |
| 303153 | U09759 | U09759 | Hs.246857 | mitogen-activated protein kinase 9 |
| 420269 | U09820 | U72937 | Hs.96264 | alpha thalassemia/mental retardation syndrome X-linked (RAD54 |
| | | | | (S. cerevisiae) homolog) |
| 102095 | U11313 | U11313 | Hs.75760 | sterol carrier protein 2 |
| 102123 | U14518 | NM_001809 | Hs.1594 | centromere protein A (17 kD) |
| 102126 | U14575 | AW950870 | Hs.78961 | protein phosphatase 1, regulatory (inhibitor) subunit 8 |
| 102133 | U15173 | AU076845 | Hs.155596 | BCL2/adenovirus E1B 19 kD-interacting protein 2 |
| 102139 | U15932 | NM_004419 | Hs.2128 | dual specificity phosphatase 5 |
| 102162 | U18291 | AA450274 | Hs.1592 | CDC16 (cell division cycle 16, S. cerevisiae, homolog) |
| 102164 | U18300 | NM_000107 | Hs.77602 | damage-specific DNA binding protein 2 (48 kD) |
| 427653 | U18383 | AA159001 | Hs.180069 | nuclear respiratory factor 1 |
| 131817 | U20536 | U20536 | Hs.3280 | caspase 6, apoptosis-related cysteine protease |
| 102200 | U21551 | AA232362 | Hs.157205 | branched chain aminotransferase 1, cytosolic |
| 102210 | U23028 | BE619413 | Hs 2437 | eukaryotic translation initiation factor 2B, subunit 5 (epsilon, |
| | | | | 82 kD) |
| 102214 | U23752 | U23752 | Hs.32964 | SRY (sex determining region Y)-box 11 |
| 132811 | U25435 | U25435 | Hs.57419 | CCCTC-binding factor (zinc finger protein) |
| 131319 | U25997 | NM_003155 | Hs.25590 | stanniocalcin 1 |
| 102256 | U28251_cds2 | U28251 | Hs 53237 | ESTs, Highly similar to Z169_HUMAN ZINC FINGER PROTEIN 169 |
| | | | | [H. sapiens] |
| 132316 | U28831 | U28831 | Hs.44566 | KIAA1641 protein |
| 102269 | U30245 | U30245 | | gb: Human myelomonocytic specific protein (MNDA) gene, 5′ |
| | | | | flanking sequence and complete exon 1. |
| 134365 | U32315 | AA568906 | Hs 82240 | syntaxin 3A |
| 102293 | U32439 | AF090116 | Hs.79348 | regulator of G-protein signalling 7 |
| 102298 | U32849 | AA382169 | Hs.54483 | N-myc (and STAT) interactor |
| 102325 | U35139 | AI815867 | Hs 50130 | necdin (mouse) homolog |
| 302344 | U36764 | BE303044 | Hs.192023 | eukaryotic translation initiation factor 3, subunit 2 (beta, 36 |
| | | | | kD) |
| 102361 | U39400 | AA223616 | Hs.75859 | chromosome 11 open reading frame 4 |
| 102367 | U39657 | U39656 | Hs.118825 | mitogen-activated protein kinase kinase 6 |
| 102388 | U41344 | AA362907 | Hs.76494 | proline arginine-rich end leucine-rich repeat protein |
| 102394 | U41766 | NM_003816 | Hs.2442 | a disintegrin and metalloproteinase domain 9 (meltrin gamma) |
| 129829 | U41813 | AF010258 | Hs 127428 | homeo box A9 |
| 102251 | U41815 | NM_004398 | Hs.41706 | DEAD/H (Asp-Glu-Ala-Asp/His) box polypeptide 10 (RNA helicase) |
| 102409 | U43286 | BE300330 | Hs.118725 | selenophosphate synthetase 2 |
| 133746 | U44378 | AW410035 | Hs.75862 | MAD (mothers against decapentaplegic, Drosophila) homolog 4 |
| 102423 | U44754 | Z47542 | Hs.179312 | small nuclear RNA activating complex, polypeptide 1, 43 kD |
| 132828 | U47011_cds1 | AB014615 | Hs.57710 | fibroblast growth factor 8 (androgen-induced) |
| 130441 | U47077 | U63630 | Hs.155637 | protein kinase, DNA-activated, catalytic polypeptide |
| 102450 | U48251 | U48251 | Hs.75871 | protein kinase C binding protein 1 |
| 129350 | U50535 | U50535 | Hs.110630 | Human BRCA2 region, mRNA sequence CG006 |
| 102534 | U56833 | U96759 | Hs.198307 | von Hippel-Lindau binding protein 1 |
| 130457 | U58091 | AB014595 | Hs.155976 | cullin 4B |
| 135065 | U58837 | AA019401 | Hs 93909 | cyclic nucleotide gated channel beta 1 |
| 102560 | U59289 | R97457 | Hs 63984 | cadherin 13, H-cadherin (heart) |
| 102567 | U59863 | U63830 | Hs.146847 | TRAF family member-associated NFKB activator |
| 134305 | U67122 | U61397 | Hs 81424 | ubiquitin-like 1 (sentrin) |
| 102638 | U67319 | U67319 | Hs.9216 | caspase 7, apoptosis-related cysteine protease |
| 132736 | U68019 | AW081883 | Hs.288261 | Homo sapiens cDNA: FLJ23037 fis, clone LNG02036, highly similar |
| | | | | to HSU68019 Homo sapiens mad protein homolog (hMAD-3) mRNA |
| 133070 | U69611 | U92649 | Hs 64311 | a disintegrin and metalloproteinase domain 17 (tumor necrosis |
| | | | | factor, alpha, converting enzyme) |
| 102663 | U70322 | NM_002270 | Hs.168075 | karyopherin (importin) beta 2 |
| 134660 | U73524 | U73524 | Hs 87465 | ATP/GTP-binding protein |
| 102735 | U79267 | AF111106 | Hs.3382 | protein phosphatase 4, regulatory subunit 1 |
| 102741 | U79291 | AW959829 | Hs.83572 | hypothetical protein MGC14433 |
| 101175 | U82671_cds2 | U82671 | Hs.36980 | melanoma antigen, family A, 2 |
| 132164 | U84573 | AI752235 | Hs.41270 | procollagen-lysine, 2-oxoglutarate 5-dioxygenase (lysine |
| | | | | hydroxylase) 2 |
| 102823 | U90914 | D85390 | Hs.5057 | carboxypeptidase D |
| 102826 | U91316 | NM_007274 | Hs.8679 | cytosolic acyl coenzyme A thioester hydrolase |
| 102831 | U91932 | AA262170 | Hs.80917 | adaptor-related protein complex 3, sigma 1 subunit |
| 102846 | U96131 | BE264974 | Hs.6566 | thyroid hormone receptor interactor 13 |
| 129777 | U97018 | U97018 | Hs.12451 | echinoderm microtubule-associated protein-like |
| 134161 | U97188 | AA634543 | Hs.79440 | IGF-II mRNA-binding protein 3 |
| 134854 | V00503 | J03464 | Hs 179573 | collagen, type I, alpha 2 |
| 302363 | X04327 | AW163799 | Hs 198365 | 2,3-bisphosphoglycerate mutase |
| 133708 | X06389 | AI018666 | Hs.75667 | synaptophysin |
| 125701 | X07496 | T72104 | Hs.93194 | apolipoprotein A-I |
| 102915 | X07820 | X07820 | Hs.2258 | matrix metalloproteinase 10 (stromelysin 2) |
| 134656 | X14787 | AI750878 | Hs 87409 | thrombospondin 1 |
| 413858 | X15525_rna1 | NM_001610 | Hs.75589 | acid phosphatase 2, lysosomal |
| 102968 | X16396 | AU076611 | Hs.154672 | methylene tetrahydrofolate dehydrogenase (NAD + dependent), |
| | | | | methenyltetrahydrofolate cyclohydrolase |
| 102971 | X16609 | X16609 | Hs.183805 | ankyrin 1, erythrocytic |
| 134037 | X53586_rna1 | AI808780 | Hs.227730 | integrin, alpha 6 |
| 103023 | X53793 | AW500470 | Hs.117950 | multifunctional polypeptide similar to SAICAR synthetase and AIR |
| | | | | carboxylase |
| 103037 | X54936 | BE018302 | Hs.2894 | placental growth factor, vascular endothelial growth factor- |
| | | | | related protein |
| 130282 | X55740 | BE245380 | Hs.153952 | 5′ nucleotidase (CD73) |
| 134542 | X57025 | M14156 | Hs 85112 | insulin-like growth factor 1 (somatomedin C) |
| 128568 | X60673_rna1 | H12912 | Hs.274691 | adenylate kinase 3 |
| 103093 | X60708 | S79876 | Hs.44926 | dipeptidylpeptidase IV (CD26, adenosine deaminase complexing |
| | | | | protein 2) |
| 133606 | X62048 | U10564 | Hs.75188 | wee1 + (S. pombe) homolog |
| 129063 | X63097 | X63094 | Hs 283822 | Rhesus blood group, D antigen |
| 424460 | X63563 | BE275979 | Hs.296014 | polymerase (RNA) II (DNA directed) polypeptide B (140 kD) |
| 133227 | X64037 | AW977263 | Hs.68257 | general transcription factor IIF, polypeptide 1 (74 kD subunit) |
| 103181 | X69636 | X69636 | Hs.334731 | Homo sapiens, clone IMAGE: 3448306, mRNA, partial cds |
| 103184 | X69878 | U43143 | Hs.74049 | fms-related tyrosine kinase 4 |
| 103194 | X70649 | NM_004939 | Hs.78580 | DEAD/H (Asp-Glu-Ala-Asp/His) box polypeptide 1 |
| 103208 | X72841 | AW411340 | Hs.31314 | retinoblastoma-binding protein 7 |
| 129698 | X74987 | BE242144 | Hs.12013 | ATP-binding cassette, sub-family E (OABP), member 1 |
| 131486 | X83107 | F06972 | Hs.27372 | BMX non-receptor tyrosine kinase |
| 130729 | X84194 | AI963747 | Hs.18573 | acylphosphatase 1, erythrocyte (common) type |
| 103334 | X85753 | NM_001260 | Hs.25283 | cyclin-dependent kinase 8 |
| 132645 | X87870 | AI654712 | Hs.54424 | hepatocyte nuclear factor 4, alpha |
| 135094 | X89066 | NM_003304 | Hs.250687 | transient receptor potential channel 1 |
| 103352 | X89398_cds2 | H09366 | Hs.78853 | uracil-DNA glycosylase |
| 103353 | X89399 | X89399 | Hs.119274 | RAS p21 protein activator (GTPase activating protein) 3 |
| | | | | (Ins(1,3,4,5)P4-binding protein) |
| 132173 | X89426 | X89426 | Hs.41716 | endothelial cell-specific molecule 1 |
| 103371 | X91247 | X91247 | Hs.13046 | thioredoxin reductase 1 |
| 131584 | X91648 | AA598509 | Hs 29117 | purine-rich element binding protein A |
| 103376 | X92098 | AL036166 | Hs 323378 | coated vesicle membrane protein |
| 103378 | X92110 | AL119690 | Hs.153618 | HCGVIII-1 protein |
| 128510 | X94703 | X94703 | Hs.296371 | RAB28, member RAS oncogene family |
| 103410 | X96506 | AA158294 | Hs.334879 | DR1-associated protein 1 (negative cofactor 2 alpha) |
| 133490 | X97230_f | AF022044 | Hs.274601 | killer cell immunoglobulin-like receptor, three domains, long |
| | | | | cytoplasmic tail, 1 |
| 103438 | X98263 | AW175781 | Hs.152720 | M-phase phosphoprotein 6 |
| 103440 | X98296 | X98296 | Hs.77578 | ubiquitin specific protease 9, X chromosome (Drosophila fat |
| | | | | facets related) |
| 103452 | X99584 | NM_006936 | Hs 85119 | SMT3 (suppressor of mif two 3, yeast) homolog 1 |
| 133536 | Y00264 | W25797.comp | Hs.177486 | amyloid beta (A4) precursor protein (protease nexin-II, Alzheimer |
| | | | | disease) |
| 135185 | Y07566 | AW404908 | Hs 96038 | Ric (Drosophila)-like, expressed in many tissues |
| 118523 | Y07759 | Y07759 | Hs 170157 | myosin VA (heavy polypeptide 12, myoxin) |
| 134662 | Y07827 | NM_007048 | Hs 284283 | butyrophilin, subfamily 3, member A1 |
| 132083 | Y07867 | BE386490 | Hs.279663 | Pirin |
| 103500 | Y09443 | AW408009 | Hs.22580 | alkylglycerone phosphate synthase |
| 134389 | Y09858 | Y09858 | Hs 82577 | spindlin-like |
| 132084 | Y12394 | NM_002267 | Hs 3886 | karyopherin alpha 3 (importin alpha 4) |
| 103540 | Z11559 | NM_002197 | Hs.154721 | aconitase 1, soluble |
| 133152 | Z11695 | Z11695 | Hs.324473 | mitogen-activated protein kinase 1 |
| 103548 | Z15005 | Z15005 | Hs.75573 | centromere protein E (312 kD) |
| 103612 | Z46261 | BE336654 | Hs.70937 | H3 histone family, member A |
| 129092 | AA011243_s | D56365 | Hs 63525 | poly(rC)-binding protein 2 |
| 103692 | AA018418 | AW137912 | Hs.227583 | Homo sapiens chromosome X map Xp11.23 L-type calcium channel |
| | | | | alpha-1 subunit (CACNA1F) gene, complete cds; HSP27 pseudogene, |
| | | | | complete sequence; and JM1 protein, JM2 protein, and Hb2E genes, |
| | | | | complete cds |
| 103695 | AA018758 | AW207152 | Hs.186600 | ESTs |
| 129796 | AA018804 | BE218319 | Hs 5807 | GTPase Rab14 |
| 132258 | AA031993 | AA306325 | Hs.4311 | SUMO-1 activating enzyme subunit 2 |
| 132683 | AA044217 | BE264633 | Hs.143638 | WD repeat domain 4 |
| 131887 | AA046548 | W17064 | Hs.332848 | SWI/SNF related, matrix associated, actin dependent regulator of |
| | | | | chromatin, subfamily e, member 1 |
| 103723 | AA057447_s | BE274312 | Hs.214783 | Homo sapiens cDNA FLJ14041 fis, clone HEMBA1005780 |
| 453368 | AA058376 | W20296 | Hs.288178 | Homo sapiens cDNA FLJ11968 fis, clone HEMBB1001133 |
| 133260 | AA083572 | AA403045 | Hs.6906 | Homo sapiens cDNA FLJ23197 fis, clone REC00917 |
| 103765 | AA085696 | AA085696 | Hs.169600 | KIAA0826 protein |
| 103766 | AA088744 | AI920783 | Hs.191435 | ESTs |
| 103767 | AA089688 | BE244667 | Hs 296155 | CGI-100 protein |
| 132051 | AA091284 | AA393968 | Hs 180145 | HSPC030 protein |
| 103773 | AA092700 | AI219323 | Hs.101077 | ESTs, Weakly similar to T22363 hypothetical protein F47G9.4 - |
| | | | | Caenorhabditis elegans [C. elegans] |
| 135289 | AA092968 | AW372569 | Hs 9788 | hypothetical protein MGC10924 similar to Nedd4 WW-binding |
| | | | | protein 5 |
| 132729 | AA094800 | AW970843 | Hs.55682 | eukaryotic translation initiation factor 3, subunit 7 (zeta, |
| | | | | 66/67 kD) |
| 103794 | AA100219 | AF244135 | Hs.30670 | hepatocellular carcinoma-associated antigen 66 |
| 131471 | AA114885 | AA164842 | Hs.192619 | KIAA1600 protein |
| 134319 | AA129547 | BE304999 | Hs.75653 | fumarate hydratase |
| 103807 | AA133016 | AW958264 | Hs.103832 | similar to yeast Upf3, variant B |
| 119159 | AA149507 | AF142419 | Hs 15020 | homolog of mouse quaking QKI (KH domain RNA binding protein) |
| 129863 | AA151005 | BE379765 | Hs.129872 | sperm associated antigen 9 |
| 103850 | AA187101 | AA187101 | Hs 213194 | hypothetical protein MGC10895 |
| 103855 | AA195179_s | W02363 | Hs.302267 | hypothetical protein FLJ10330 |
| 322026 | AA203138 | AW024973 | Hs 283675 | NPD009 protein |
| 135300 | AA203645 | AA142922 | Hs.278626 | Arg/Abl-interacting protein ArgBP2 |
| 103861 | AA206236 | AA206236 | Hs.4944 | hypothetical protein FLJ12783 |
| 130634 | AA227621 | AI769067 | Hs.127824 | ESTs, Weakly similar to T28770 hypothetical protein W03D2.1 - |
| | | | | Caenorhabditis elegans [C. elegans] |
| 447735 | AA248283 | AA775268 | Hs.6127 | Homo sapiens cDNA: FLJ23020 fis, clone LNG00943 |
| 103909 | AA249611 | AA249611 | Hs.47438 | SH3 domain binding glutamic acid-rich protein |
| 131236 | AA282640 | AF043117 | Hs 24594 | ubiquitination factor E4B (homologous to yeast UFD2) |
| 134060 | AA287199 | D42039 | Hs.78871 | mesoderm development candidate 2 |
| 129013 | AA313990 | AA371156 | Hs.107942 | DKFZP564M112 protein |
| 129435 | AA314256 | AF151852 | Hs.111449 | CGI-94 protein |
| 103988 | AA314389 | AA314389 | Hs 42500 | ADP-ribosylation factor-like 5 |
| 104000 | AA324364 | AI146527 | Hs.80475 | polymerase (RNA) II (DNA directed) polypeptide J (13.3 kD) |
| 425284 | AA329211_s | AF155568 | Hs 155489 | NS1-associated protein 1 |
| 128629 | AA399187 | AL096748 | Hs.102708 | DKFZP434A043 protein |
| 133281 | AA421079 | AK001601 | Hs.69594 | high-mobility group 20A |
| 104104 | AA422029 | AA422029 | Hs.143640 | ESTs, Weakly similar to hyperpolarization-activated cyclic |
| | | | | nucleotide-gated channel hHCN2 [H. sapiens] |
| 108154 | AA425230 | NM_005754 | Hs.220689 | Ras-GTPase-activating protein SH3-domain-binding protein |
| 132091 | AA447052 | AW954243 | Hs.170218 | KIAA0251 protein |
| 135073 | AA452000 | W55956 | Hs.94030 | Homo sapiens mRNA; cDNA DKFZp586E1624 (from clone DKFZp586E1624) |
| 131367 | AA456687 | AI750575 | Hs 173933 | nuclear factor I/A |
| 129593 | AA487015_s | AI338247 | Hs.98314 | Homo sapiens mRNA; cDNA DKFZp586L0120 (from clone DKFZp586L0120) |
| 135266 | AB002326 | R41179 | Hs 97393 | KIAA0328 protein |
| 133505 | C01527 | AI630124 | Hs.324504 | Homo sapiens mRNA; cDNA DKFZp586J0720 (from clone DKFZp586J0720) |
| 132064 | C01714 | AA121098 | Hs 3838 | serum-inducible kinase |
| 134393 | C01811_f | W52642 | Hs.8261 | hypothetical protein FLJ22393 |
| 131427 | C02352_s | AF151879 | Hs.26706 | CGI-121 protein |
| 133435 | C02375 | AI929357 | Hs.323966 | Homo sapiens clone H63 unknown mRNA |
| 104282 | C14448 | C14448 | Hs 332338 | EST |
| 134827 | D16611_s | BE314037 | Hs.89866 | coproporphyrinogen oxidase (coproporphyria, harderoporphyria) |
| 130443 | D25216 | D25216 | Hs.155650 | KIAA0014 gene product |
| 131742 | D31352 | AA961420 | Hs.31433 | ESTs |
| 132837 | D58024_s | AA370362 | Hs 57958 | EGF-TM7-latrophilin-related protein |
| 130377 | D80897 | NM_014909 | Hs 155182 | KIAA1036 protein |
| 104334 | D82614 | D82614 | Hs.78771 | phosphoglycerate kinase 1 |
| 134593 | D87845 | NM_000437 | Hs.234392 | platelet-activating factor acetylhydrolase 2 (40 kD) |
| 134731 | D89377_i | D89377 | Hs.89404 | msh (Drosophila) homeo box homolog 2 |
| 129913 | H06583 | NM_001310 | Hs 13313 | cAMP responsive element binding protein-like 2 |
| 131670 | H40732 | H03514 | Hs.10130 | ESTs |
| 104394 | H46617 | AA129551 | Hs.172129 | Homo sapiens cDNA. FLJ21409 fis, clone COL03924 |
| 104402 | H56731 | H56731 | Hs.132956 | ESTs |
| 129781 | H75570 | AA306090 | Hs 124707 | ESTs |
| 129077 | H78886 | N74724 | Hs.108479 | ESTs |
| 104417 | H81241 | AI819448 | Hs.320861 | Kruppel-like factor 8 |
| 134927 | L36531 | L36531 | Hs.91296 | integrin, alpha 8 |
| 129280 | M63154 | M63154 | Hs.110014 | gastric intrinsic factor (vitamin B synthesis) |
| 134498 | M63180 | AW246273 | Hs.84131 | threonyl-tRNA synthetase |
| 104460 | M91504 | AW955705 | Hs 62604 | Homo sapiens, clone IMAGE: 4299322, mRNA, partial cds |
| 104488 | N56191 | N56191 | Hs.106511 | protocadherin 17 |
| 131248 | N78483 | AI038989 | Hs.332633 | Bardet-Biedl syndrome 2 |
| 129214 | N79268 | AL044335 | Hs 109526 | zinc finger protein 198 |
| 130017 | R14652 | AK000096 | Hs.143198 | inhibitor of growth family, member 3 |
| 104530 | R20459 | AK001676 | Hs.12457 | hypothetical protein FLJ10814 |
| 104534 | R22303 | R22303 | | gb: yh26b09.r1 Soares placenta Nb2HP Homo sapiens cDNA clone |
| | | | | IMAGE: 130841 5′, mRNA sequence. |
| 104544 | R33779 | AI091173 | Hs.222362 | ESTs, Weakly similar to p40 [H. sapiens] |
| 133328 | R36553 | AW452738 | Hs.265327 | hypothetical protein DKFZp761I141 |
| 104567 | R64534 | AA040620 | Hs.5672 | hypothetical protein AF140225 |
| 128562 | R66475 | AA923382 | Hs.101490 | ESTs |
| 129575 | R70621 | F08282 | Hs.278428 | progestin induced protein |
| 130776 | R79356 | AF167706 | Hs.19280 | cysteine-rich motor neuron 1 |
| 104599 | R84933 | AW815036 | Hs 151251 | ESTs |
| 104660 | RC_AA007160 | BE298665 | Hs.14846 | Homo sapiens mRNA; cDNA DKFZp564D016 (from clone DKFZp564D016) |
| 104667 | RC_AA007234_s | AI239923 | Hs.30098 | ESTs |
| 104718 | RC_AA018409 | AI143020 | Hs.36250 | ESTs, Weakly similar to I38022 hypothetical protein |
| | | | | [H. sapiens] |
| 104764 | RC_AA025351 | AI039243 | Hs 278585 | ESTs |
| 104786 | RC_AA027168 | AA027167 | Hs.10031 | KIAA0955 protein |
| 104787 | RC_AA027317 | AA027317 | | gb: ze97d11 s1 Soares_fetal_heart_NbHH19W Homo sapiens cDNA clone |
| | | | | IMAGE: 366933 3′ similar to contains Alu repetitive element;, |
| | | | | mRNA sequence. |
| 134079 | RC_AA029423 | AK001751 | Hs.171835 | hypothetical protein FLJ10889 |
| 104804 | RC_AA031357 | AI858702 | Hs 31803 | ESTs, Weakly similar to N-WASP [H. sapiens] |
| 104865 | RC_AA045136 | T79340 | Hs.22575 | B-cell CLL/lymphoma 6, member B (zinc finger protein) |
| 130828 | RC_AA053400 | AW631469 | Hs 203213 | ESTs |
| 104907 | RC_AA055829 | AA055829 | Hs 196701 | ESTs, Weakly similar to ALU1_HUMAN ALU SUBFAMILY J SEQUENCE |
| | | | | CONTAMINATION WARNING ENTRY [H. sapiens] |
| 104943 | RC_AA065217 | AF072873 | Hs 114218 | frizzled (Drosophila) homolog 6 |
| 105013 | RC_AA116054 | H63789 | Hs 296288 | ESTs, Weakly similar to KIAA0638 protein [H sapiens] |
| 105024 | RC_AA126311 | AA126311 | Hs.9879 | ESTs |
| 132592 | RC_AA129390 | AW803564 | Hs.288850 | Homo sapiens cDNA; FLJ22528 fis, clone HRC12825 |
| 105038 | RC_AA130273 | AW503733 | Hs.9414 | KIAA1488 protein |
| 105077 | RC_AA142919 | W55946 | Hs 234863 | Homo sapiens cDNA FLJ12082 fis, clone HEMBB1002492 |
| 105096 | RC_AA150205 | AL042506 | Hs.21599 | Kruppel-like factor 7 (ubiquitous) |
| 129215 | RC_AA176867 | AB040930 | Hs.126085 | KIAA1497 protein |
| 105169 | RC_AA180321 | BE245294 | Hs.180789 | S164 protein |
| 132796 | RC_AA180487 | NM_006283 | Hs.173159 | transforming, acidic coiled-coil containing protein 1 |
| 130401 | RC_AA187634 | BE396283 | Hs.173987 | eukaryotic translation initiation factor 3, subunit 1 (alpha, |
| | | | | 35 kD) |
| 105200 | RC_AA195399 | AA328102 | Hs.24641 | cytoskeleton associated protein 2 |
| 130114 | RC_AA234717 | AA233393 | Hs.14992 | hypothetical protein FLJ11151 |
| 105330 | RC_AA234743 | AW338625 | Hs.22120 | ESTs |
| 105337 | RC_AA234957 | AI468789 | Hs.23200 | myotubularin related protein 1 |
| 129385 | RC_AA235604 | AA172106 | Hs.110950 | Rag C protein |
| 105376 | RC_AA236559 | AW994032 | Hs.8768 | hypothetical protein FLJ10849 |
| 105397 | RC_AA242868 | AA814807 | Hs.7395 | hypothetical protein FLJ23182 |
| 131962 | RC_AA251776 | AK000046 | Hs.267448 | hypothetical protein FLJ20039 |
| 131991 | RC_AA251909 | AF053306 | Hs.36708 | budding uninhibited by benzimidazoles 1 (yeast homolog), beta |
| 128658 | RC_AA252672_s | BE397354 | Hs.324830 | diptheria toxin resistance protein required for diphthamide |
| | | | | biosynthesis (Saccharomyces)-like 2 |
| 105489 | RC_AA256157 | AA256157 | Hs 24115 | Homo sapiens cDNA FLJ14178 fis, clone NT2RP2003339 |
| 105508 | RC_AA256680 | AA173942 | Hs.326416 | Homo sapiens mRNA; cDNA DKFZp564H1916 (from clone DKFZp564H1916) |
| 105539 | RC_AA258873 | AB040884 | Hs.109694 | KIAA1451 protein |
| 135172 | RC_AA262727 | AB028956 | Hs.12144 | KIAA1033 protein |
| 131569 | RC_AA281451 | AL389951 | Hs.271623 | nucleoporin 50 kD |
| 132542 | RC_AA281545 | AL137751 | Hs 263671 | Homo sapiens mRNA; cDNA DKFZp434I0812 (from clone DKFZp434I0812), |
| | | | | partial cds |
| 105643 | RC_AA282069 | BE621719 | Hs.173802 | KIAA0603 gene product |
| 105659 | RC_AA283044 | AA283044 | Hs.25625 | hypothetical protein FLJ11323 |
| 105666 | RC_AA283930 | AA426234 | Hs.34906 | ESTs, Weakly similar to T17210 hypothetical protein |
| | | | | DKFZp434N041.1 [H sapiens] |
| 105674 | RC_AA284755 | AI609530 | Hs.279789 | histone deacetylase 3 |
| 105709 | RC_AA291268 | AI928962 | Hs.26761 | DKFZP586L0724 protein |
| 105722 | RC_AA291927 | AI922821 | Hs.32433 | ESTs |
| 105765 | RC_AA343514 | AA299688 | Hs 24183 | ESTs |
| 115951 | RC_AA398109 | BE546245 | Hs 301048 | sec13-like protein |
| 105962 | RC_AA405737 | AW880358 | Hs.339808 | hypothetical protein FLJ10120 |
| 105985 | RC_AA406610 | AA406610 | | gb: zv15b10.s1 Soares_NhHMPu_S1 Homo sapiens cDNA clone |
| | | | | IMAGE: 753691 3′ similar to gb: X02067 |
| 106008 | RC_AA411465 | AB033888 | Hs.8619 | SRY (sex determining region Y)-box 18 |
| 131216 | RC_AA416886 | AI815486 | Hs.243901 | Homo sapiens cDNA FLJ20738 fis, clone HEP08257 |
| 134222 | RC_AA424013 | AW855861 | Hs.8025 | Homo sapiens clone 23767 and 23782 mRNA sequences |
| 113689 | RC_AA424148 | AB037850 | Hs.16621 | DKFZP434I116 protein |
| 106141 | RC_AA424558 | AF031463 | Hs 9302 | phosducin-like |
| 130839 | RC_AA424961_s | AB011169 | Hs.20141 | similar to S. cerevisiae SSM4 |
| 106157 | RC_AA425367 | W37943 | Hs.34892 | KIAA1323 protein |
| 130777 | RC_AA425921 | AW135049 | Hs.285418 | Homo sapiens cDNA FLJ10643 fis, clone NT2RP2005753, highly |
| | | | | similar to Homo sapiens I-1 receptor |
| 130561 | RC_AA426220 | AB011095 | Hs.16032 | KIAA0523 protein |
| 106196 | RC_AA427735 | AA525993 | Hs.173699 | ESTs, Weakly similar to ALU1_HUMAN ALU SUBFAMILY J SEQUENCE |
| | | | | CONTAMINATION WARNING |
| 131878 | RC_AA430673 | AA083764 | Hs.6101 | hypothetical protein MGC3178 |
| 133200 | RC_AA432248 | AB037715 | Hs.183639 | hypothetical protein FLJ10210 |
| 106302 | RC_AA435896 | AA398859 | Hs.18397 | hypothetical protein FLJ23221 |
| 106328 | RC_AA436705 | AL079559 | Hs 28020 | KIAA0766 gene product |
| 450534 | RC_AA446561 | AI570189 | Hs.25132 | KIAA0470 gene product |
| 106423 | RC_AA448238 | AB020722 | Hs.16714 | Rho guanine exchange factor (GEF) 15 |
| 133442 | RC_AA448688 | AL137663 | Hs.7378 | Homo sapiens mRNA; cDNA DKFZp434G227 (from clone DKFZp434G227) |
| 439608 | RC_AA449756 | AW864696 | Hs 301732 | hypothetical protein MGC5306 |
| 106477 | RC_AA450303 | R23324 | Hs.41693 | DnaJ (Hsp40) homolog, subfamily B, member 4 |
| 106503 | RC_AA452411 | AB033042 | Hs.29679 | cofactor required for Sp1 transcriptional activation, subunit 3 |
| | | | | (130 kD) |
| 446999 | RC_AA454566 | AA151520 | Hs.334822 | hypothetical protein MGC4485 |
| 106543 | RC_AA454667 | AA676939 | Hs.69285 | neuropilin 1 |
| 130010 | RC_AA456437 | AA301116 | Hs.142838 | nucleolar phosphoprotein Nopp34 |
| 106589 | RC_AA456646 | AK000933 | Hs.28661 | Homo sapiens cDNA FLJ10071 fis, clone HEMBA1001702 |
| 106593 | RC_AA456826 | AW296451 | Hs.24605 | ESTs |
| 106596 | RC_AA456981 | AA452379 | Hs.293552 | ESTs, Moderately similar to ALU7_HUMAN ALU SUBFAMILY SQ SEQUENCE |
| | | | | CONTAMINATION |
| 134655 | RC_AA458959 | AF265208 | Hs.123090 | SWI/SNF related, matnx associated, actin dependent regulator of |
| | | | | chromatin, subfamily f, member 1 |
| 106636 | RC_AA459950 | AW958037 | Hs 286 | ribosomal protein L4 |
| 106654 | RC_AA460449 | AW075485 | Hs.286049 | phosphoserine aminotransferase |
| 131353 | RC_AA463910 | AW754182 | | gb: RC2-CT0321-131199-011-c01 CT0321 Homo sapiens cDNA, mRNA |
| | | | | sequence |
| 106707 | RC_AA464603 | AK000566 | Hs.98135 | hypothetical protein FLJ20559 |
| 131710 | RC_AA464606 | NM_015368 | Hs 30985 | pannexin 1 |
| 106717 | RC_AA465093 | AA600357 | Hs.239489 | TIA1 cytotoxic granule-associated RNA-binding protein |
| 131775 | RC_AA465692 | AB014548 | Hs 31921 | KIAA0648 protein |
| 106747 | RC_AA476473 | NM_007118 | Hs.171957 | triple functional domain (PTPRF interacting) |
| 106773 | RC_AA478109 | AA478109 | Hs.188833 | ESTs |
| 106781 | RC_AA478474 | AA330310 | Hs 24181 | ESTs |
| 106817 | RC_AA480889 | D61216 | Hs.18672 | ESTs |
| 106846 | RC_AA485223 | AB037744 | Hs 34892 | KIAA1323 protein |
| 106848 | RC_AA485254 | AA449014 | Hs 121025 | chromosome 11 open reading frame 5 |
| 106856 | RC_AA486183 | W58353 | Hs.285123 | Homo sapiens mRNA full length insert cDNA clone EUROIMAGE 2005779 |
| 418699 | RC_AA496936 | BE539639 | Hs.173030 | ESTs, Weakly similar to ALU8_HUMAN ALU SUBFAMILY SX SEQUENCE |
| | | | | CONTAMINATION WARNING |
| 107001 | RC_AA598589 | AI926520 | Hs.31016 | putative DNA binding protein |
| 130638 | RC_AA598831_f | AW021276 | Hs.17121 | ESTs |
| 107054 | RC_AA600150 | AI076459 | Hs.15978 | KIAA1272 protein |
| 107059 | RC_AA608545 | BE614410 | Hs.23044 | RAD51 (S. cerevisiae) homolog (E coli RecA homolog) |
| 107080 | RC_AA609210 | AL122043 | Hs 19221 | hypothetical protein DKFZp566G1424 |
| 107115 | RC_AA610108 | BE379623 | Hs.27693 | peptidylprolyl isomerase (cyclophilin)-like 1 |
| 107130 | RC_AA620582 | AB033106 | Hs.12913 | KIAA1280 protein |
| 107156 | RC_AA621239 | AA137043 | Hs.9663 | programmed cell death 6-interacting protein |
| 107174 | RC_AA621714 | BE122762 | Hs.25338 | ESTs |
| 130621 | RC_AA621718 | AW513087 | Hs.16803 | LUC7 (S. cerevisiae)-like |
| 107190 | RC_D19673 | AA836401 | Hs.5103 | ESTs |
| 132626 | RC_D25755_s | AW504732 | Hs.21275 | hypothetical protein FLJ11011 |
| 107217 | RC_D51095 | AL080235 | Hs.35861 | DKFZP586E1621 protein |
| 131610 | RC_D60272_i | AA357879 | Hs.29423 | scavenger receptor with C-type lectin |
| 129604 | T08879 | AF088886 | Hs.11590 | cathepsin F |
| 107295 | T34527 | AA186629 | Hs.80120 | UDP-N-acetyl-alpha-D-galactosamine.polypeptide N- |
| | | | | acetylgalactosaminyltransferase 1 (GalNAc-T1) |
| 107299 | T40327_s | BE277457 | Hs.30661 | hypothetical protein MGC4606 |
| 107315 | T62771_s | AA316241 | Hs.90691 | nucleophosmin/nucleoplasmin 3 |
| 107316 | T63174_s | T63174 | Hs.193700 | Homo sapiens mRNA; cDNA DKFZp586I0324 (from clone DKFZp586I0324) |
| 107328 | T83444 | AW959891 | Hs.76591 | KIAA0887 protein |
| 107334 | T93641 | T93597 | Hs.187429 | ESTs |
| 134715 | U48263 | U48263 | Hs.89040 | prepronociceptin |
| 128636 | U49065 | U49065 | Hs.102865 | interleukin 1 receptor-like 2 |
| 129938 | U79300 | AW003668 | Hs 135587 | Human clone 23629 mRNA sequence |
| 107375 | U88573 | BE011845 | Hs.251064 | high-mobility group (nonhistone chromosomal) protein 14 |
| 130074 | U93867 | AL038596 | Hs.250745 | polymerase (RNA) III (DNA directed) (62 kD) |
| 107387 | W01094 | D86983 | Hs.118893 | Melanoma associated gene |
| 132036 | W01568 | AL157433 | Hs.37706 | hypothetical protein DKFZp434E2220 |
| 107426 | W26853 | W26853 | Hs.291003 | hypothetical protein MGC4707 |
| 113857 | W27179 | AW243158 | Hs.5297 | DKFZP564A2416 protein |
| 135388 | W27965 | W27965 | Hs.99865 | epimorphin |
| 130419 | W36280_s | AF037448 | Hs.155489 | NS1-associated protein 1 |
| 107469 | W47063 | W47063 | Hs.94668 | ESTs |
| 132616 | W79060 | BE262677 | Hs.283558 | hypothetical protein PRO1855 |
| 107506 | W88550 | AB028981 | Hs.8021 | KIAA1058 protein |
| 132358 | X60486 | NM_003542 | Hs.46423 | H4 histone family, member G |
| 107522 | X78931_s | X78931 | Hs 99971 | zinc finger protein 272 |
| 125827 | Z14077_s | NM_003403 | Hs.97496 | YY1 transcription factor |
| 107582 | RC_AA002147 | AA002147 | Hs.59952 | EST |
| 107609 | RC_AA004711 | R75654 | Hs.164797 | hypothetical protein FLJ13693 |
| 107661 | RC_AA010383 | AA010383 | Hs 60389 | ESTs |
| 107714 | RC_AA015761 | AA015761 | Hs 60642 | ESTs |
| 107775 | RC_AA018772 | AW008846 | Hs.60857 | ESTs |
| 107832 | RC_AA021473_r | AA021473 | | gb: ze66c11.s1 Soares retina N2b4HR Homo sapiens cDNA clone |
| | | | | IMAGE: 363956 3′, mRNA sequence |
| 107859 | RC_AA024835 | AW732573 | Hs.47584 | potassium voltage-gated channel, delayed-rectifier, subfamily S, |
| | | | | member 3 |
| 124337 | RC_AA025858 | N23541 | Hs 281561 | Homo sapiens cDNA: FLJ23582 fis, clone LNG13759 |
| 107914 | RC_AA027229 | AA027229 | Hs.61329 | ESTs, Weakly similar to T16370 hypothetical protein F45E12.5 - |
| | | | | Caenorhabditis elegans [C. elegans] |
| 107935 | RC_AA029428 | AA029428 | Hs 61555 | ESTs |
| 116262 | RC_AA035143 | AI936442 | Hs.59838 | hypothetical protein FLJ10808 |
| 131461 | RC_AA035237 | AA992841 | Hs.27263 | KIAA1458 protein |
| 108007 | RC_AA039347 | AA039347 | Hs 61916 | EST |
| 108029 | RC_AA040740 | AA040740 | Hs.62007 | ESTs |
| 108040 | RC_AA041551 | AL121031 | Hs 159971 | SWI/SNF related, matrix associated, actin dependent regulator of |
| | | | | chromatin, subfamily b, member 1 |
| 108084 | RC_AA045513 | AA058944 | Hs.116602 | Homo sapiens, clone IMAGE: 4154008, mRNA, partial cds |
| 108088 | RC_AA045745 | AA045745 | Hs 62886 | ESTs |
| 108168 | RC_AA055348 | AI453137 | Hs.63176 | ESTs |
| 130719 | RC_AA056582_s | AA679262 | Hs.14235 | hypothetical protein FLJ20008, KIAA1839 protein |
| 108189 | RC_AA056697 | AW376061 | Hs.63335 | ESTs, Moderately similar to A46010 X-linked retinopathy protein |
| | | | | [H. sapiens] |
| 108190 | RC_AA056746 | AA056746 | Hs.63338 | EST |
| 108203 | RC_AA057678 | AW847814 | Hs.289005 | Homo sapiens cDNA: FLJ21532 fis, clone COL06049 |
| 108216 | RC_AA058681 | AA524743 | Hs.44883 | ESTs |
| 108217 | RC_AA058686 | AA058686 | Hs.62588 | ESTs |
| 108245 | RC_AA062840 | BE410285 | Hs 89545 | proteasome (prosome, macropain) subunit, beta type, 4 |
| 108277 | RC_AA064859 | AA064859 | | gb: zm50f03.s1 Stratagene fibroblast (937212) Homo sapiens cDNA |
| | | | | clone IMAGE: 529085 3′, mRNA |
| 108280 | RC_AA065069 | AA065069 | | gb: zm12e11.s1 Stratagene pancreas (937208) Homo sapiens cDNA |
| | | | | clone 3′, mRNA sequence |
| 108309 | RC_AA069923 | AA069818 | | gb: zm67e03.r1 Stratagene neuroepithelium (937231) Homo sapiens |
| | | | | cDNA clone 5′ similar to |
| 133739 | RC_AA070799_s | BE536554 | Hs.278270 | unactive progesterone receptor, 23 kD |
| 108340 | RC_AA070815 | AA069820 | Hs.180909 | peroxiredoxin 1 |
| 108403 | RC_AA075374 | AA075374 | | gb: zm87a01.s1 Stratagene ovarian cancer (937219) Homo sapiens |
| | | | | cDNA clone IMAGE: 544872 3′, mRNA sequence. |
| 108427 | RC_AA076382 | AA076382 | | gb: zm91g08.s1 Stratagene ovarian cancer (937219) Homo sapiens |
| | | | | cDNA clone IMAGE: 545342 3′, mRNA sequence. |
| 108435 | RC_AA078787 | T82427 | Hs.194101 | Homo sapiens cDNA: FLJ20869 fis, clone ADKA02377 |
| 108439 | RC_AA078986 | AA078986 | | gb: zm92h01.s1 Stratagene ovarian cancer (937219) Homo sapiens |
| | | | | cDNA clone IMAGE 545425 3′, mRNA sequence. |
| 108465 | RC_AA079393 | AA079393 | Hs.3462 | cytochrome c oxidase subunit VIIc |
| 108469 | RC_AA079487 | AA079487 | | gb: zm97f08.s1 Stratagene colon HT29 (937221) Homo sapiens cDNA |
| | | | | clone 3′, mRNA sequence |
| 108500 | RC_AA083207 | AA083207 | Hs.68270 | EST |
| 108501 | RC_AA083256 | AA083256 | | gb: zn08g12.s1 Stratagene hNT neuron (937233) Homo sapiens cDNA |
| | | | | clone 3′ similar to gb: M33308 |
| 108533 | RC_AA084415 | AA084415 | | gb: zn06g09.s1 Stratagene hNT neuron (937233) Homo sapiens cDNA |
| | | | | clone IMAGE: 546688 3′, mRNA |
| 108562 | RC_AA085274 | AA100796 | | gb: zm26c06.s1 Stratagene pancreas (937208) Homo sapiens cDNA |
| | | | | clone 3′ similar to gb: X15341 |
| 108589 | RC_AA088678 | AI732404 | Hs.68846 | ESTs |
| 130890 | RC_AA100925 | AI907537 | Hs.76698 | stress-associated endoplasmic reticulum protein 1; ribosome |
| | | | | associated membrane protein 4 |
| 134585 | RC_AA101255 | D14041 | Hs.278573 | H-2K binding factor-2 |
| 130385 | RC_AA126474 | AW067800 | Hs.155223 | stanniocalcin 2 |
| 108749 | RC_AA127017 | AA127017 | Hs.71052 | ESTs |
| 108807 | RC_AA129968 | AI652236 | Hs 49376 | hypothetical protein FLJ20644 |
| 108808 | RC_AA130240 | AA045088 | Hs.62738 | ESTs |
| 108833 | RC_AA131866 | AF188527 | Hs 61661 | ESTs, Weakly similar to AF174605 1 F-box protein Fbx25 |
| | | | | [H. sapiens] |
| 107290 | RC_AA132039 | W27740 | Hs 323780 | ESTs |
| 108846 | RC_AA132983 | AL117452 | Hs.44155 | DKFZP586G1517 protein |
| 108857 | RC_AA133250 | AK001468 | Hs.62180 | anillin (Drosophila Scraps homolog), actin binding protein |
| 131474 | RC_AA133583_s | L46353 | Hs.2726 | high-mobility group (nonhistone chromosomal) protein isoform I-C |
| 108894 | RC_AA135941 | AK001431 | Hs.5105 | hypothetical protein FLJ10569 |
| 108941 | RC_AA148650 | AA148650 | | gb: zo09e06.s1 Stratagene neuroepithelium NT2RAMI 937234 |
| | | | | Homo sapiens cDNA clone IMAGE 567202 3′, |
| 108968 | RC_AA151110 | AI304870 | Hs.188680 | ESTs |
| 108996 | RC_AA155754 | AW995610 | Hs.332436 | EST |
| 109001 | RC_AA156125 | AI056548 | Hs.72116 | hypothetical protein FLJ20992 similar to hedgehog-interacting |
| | | | | protein |
| 131183 | RC_AA156289 | AI611807 | Hs.285107 | hypothetical protein FLJ13397 |
| 109019 | RC_AA156997 | AA156755 | Hs.72150 | ESTs |
| 109022 | RC_AA157291 | AA157291 | Hs.21479 | ubinuclein 1 |
| 109023 | RC_AA157293 | AA157293 | Hs.72168 | ESTs |
| 109068 | RC_AA164293_f | AA164293 | Hs.72545 | ESTs |
| 109072 | RC_AA164676 | AI732585 | Hs 22394 | hypothetical protein FLJ10893 |
| 129021 | RC_AA167375 | AL044675 | Hs 173081 | KIAA0530 protein |
| 130346 | RC_AA167550 | H05769 | Hs.188757 | Homo sapiens, clone MGC: 5564, mRNA, complete cds |
| 109146 | RC_AA176589 | AA176589 | Hs.142078 | EST |
| 109172 | RC_AA180448 | AA180448 | Hs.144300 | EST |
| 131080 | RC_AA187144_s | NM_001955 | Hs 2271 | endothelin 1 |
| 129208 | RC_AA189170_f | AI587376 | Hs 109441 | MSTP033 protein |
| 109222 | RC_AA192757 | AA192833 | Hs 333512 | similar to rat myomegalin |
| 109300 | RC_AA205650 | AA418276 | Hs.170142 | ESTs |
| 109481 | RC_AA233342 | AA878923 | Hs.289069 | hypothetical protein FLJ21016 |
| 109485 | RC_AA233472 | BE619092 | Hs.28465 | Homo sapiens cDNA: FLJ21869 fis, clone HEP02442 |
| 109516 | RC_AA234110 | AI471639 | Hs.71913 | ESTs |
| 109537 | RC_D80981 | AI858695 | Hs 34898 | ESTs |
| 109556 | RC_F01660 | AI925294 | Hs 87385 | ESTs |
| 109577 | RC_F02206 | F02206 | Hs.296639 | Homo sapiens potassium channel subunit (HERG-3) mRNA, complete |
| | | | | cds |
| 109578 | RC_F02208 | F02208 | Hs.27214 | ESTs |
| 109595 | RC_F02544 | AA078629 | Hs 27301 | ESTs |
| 109625 | RC_F03918 | H29490 | Hs.22697 | ESTs |
| 131983 | RC_F04258_s | AF119665 | Hs.184011 | pyrophosphatase (inorganic) |
| 109648 | RC_F04600 | H17800 | Hs.7154 | ESTs |
| 109671 | RC_F08998 | R59210 | Hs.26634 | ESTs |
| 109699 | RC_F09605 | H18013 | Hs.167483 | ESTs |
| 109820 | RC_F11115 | AW016809 | Hs.323795 | ESTs |
| 109933 | RC_H06371 | R52417 | Hs.20945 | Homo sapiens clone 24993 mRNA sequence |
| 110014 | RC_H10995 | AL109666 | Hs 7242 | Homo sapiens mRNA full length insert cDNA clone EUROIMAGE 35907 |
| 110039 | RC_H11938 | H11938 | Hs.21907 | histone acetyltransferase |
| 110099 | RC_H16568 | R44557 | Hs.23748 | ESTs |
| 110107 | RC_H16772 | AW151660 | Hs.31444 | ESTs |
| 110155 | RC_H18951 | AI559626 | Hs 93522 | Homo sapiens mRNA for KIAA1647 protein, partial cds |
| 110197 | RC_H20859 | AW090386 | Hs.112278 | arrestin, beta 1 |
| 110223 | RC_H23747 | H19836 | Hs 31697 | ESTs |
| 110306 | RC_H38087 | H38087 | Hs 105509 | CTL2 gene |
| 110335 | RC_H40331 | H65490 | Hs.18845 | ESTs |
| 110342 | RC_H40567 | H40961 | Hs.33008 | ESTs |
| 110395 | RC_H46966 | AA025116 | Hs.33333 | ESTs |
| 110511 | RC_H56640_i | H56640 | Hs.221460 | ESTs |
| 110523 | RC_H57154 | AI040384 | Hs.19102 | ESTs, Weakly similar to organic anion transporter 1 |
| | | | | [H. sapiens] |
| 110715 | RC_H96712 | H96712 | Hs 269029 | ESTs |
| 110754 | RC_N20814 | AW302200 | Hs.6336 | KIAA0672 gene product |
| 130132 | RC_N25249 | U55936 | Hs.184376 | synaptosomal-associated protein, 23 kD |
| 131135 | RC_N27100 | NM_016569 | Hs.267182 | TBX3-iso protein |
| 134263 | RC_N39616 | AW973443 | Hs.8086 | RNA (guanine-7-) methyltransferase |
| 110938 | RC_N48982 | N48982 | Hs.38034 | Homo sapiens cDNA FLJ12924 fis, clone NT2RP2004709 |
| 110983 | RC_N51957 | NM_015367 | Hs.10267 | MIL1 protein |
| 115062 | RC_N52271 | AA253314 | Hs.154103 | LIM protein (similar to rat protein kinase C-binding enigma) |
| 111081 | RC_N59435 | AI146349 | Hs.271614 | CGI-112 protein |
| 111128 | RC_N64139 | AW505364 | Hs.19074 | LATS (large tumor suppressor, Drosophila) homolog 2 |
| 135244 | RC_N66981 | AI834273 | Hs.9711 | novel protein |
| 111216 | RC_N68640 | AW139408 | Hs.152940 | ESTs |
| 437562 | RC_N69352 | AB001636 | Hs 5683 | DEAD/H (Asp-Glu-Ala-Asp/His) box polypeptide 15 |
| 131002 | RC_N95226 | AL050295 | Hs.22039 | KIAA0758 protein |
| 111399 | RC_R00138 | AW270776 | Hs 18857 | ESTs |
| 111514 | RC_R07998 | R07998 | | gb: yf16g11.s1 Soares fetal liver spleen 1NFLS Homo sapiens cDNA |
| | | | | clone IMAGE: 127076 3′ similar to |
| 130182 | RC_R08929 | BE267033 | Hs.192853 | ubiquitin-conjugating enzyme E2G 2 (homologous to yeast UBC7) |
| 111574 | RC_R10307 | AI024145 | Hs.188526 | ESTs |
| 111804 | RC_R33354 | AA482478 | Hs 181785 | ESTs |
| 111831 | RC_R36083 | R36095 | Hs.268695 | ESTs |
| 129675 | RC_R37938_f | NM_015556 | Hs.172180 | KIAA0440 protein |
| 111904 | RC_R39330 | Z41572 | | gb: HSCZYB122 normalized infant brain cDNA Homo sapiens cDNA clone |
| | | | | c-zyb12, mRNA sequence |
| 133868 | RC_R40816_s | AB012193 | Hs.183874 | cullin 4A |
| 112033 | RC_R43162_s | R49031 | Hs.22627 | ESTs |
| 130987 | RC_R45698 | BE613269 | Hs 21893 | hypothetical protein DKFZp761N0624 |
| 112300 | RC_R54554 | H24334 | Hs.26125 | ESTs |
| 112513 | RC_R68425 | R68425 | Hs.13809 | hypothetical protein FLJ10648 |
| 112514 | RC_R68568 | R68568 | Hs.183373 | src homology 3 domain-containing protein HIP-55 |
| 112522 | RC_R68763 | R68857 | Hs 265499 | ESTs |
| 112540 | RC_R70467 | R69751 | | gb: yi40a10.s1 Soares placenta Nb2HP Homo sapiens cDNA clone 3′, |
| | | | | mRNA sequence |
| 130346 | RC_R73565 | H05769 | Hs.188757 | Homo sapiens, clone MGC: 5564, mRNA, complete cds |
| 129534 | RC_R73640 | AK002126 | Hs.11260 | hypothetical protein FLJ11264 |
| 112597 | RC_R78376 | R78376 | Hs.29733 | EST |
| 112732 | RC_R92453 | R92453 | Hs 34590 | ESTs |
| 131458 | RC_T03865 | BE297567 | Hs.27047 | hypothetical protein FLJ20392 |
| 112888 | RC_T03872 | AW195317 | Hs.107716 | hypothetical protein FLJ22344 |
| 131863 | RC_T10072 | AI656378 | Hs.33461 | ESTs |
| 112911 | RC_T10080 | AW732747 | Hs.13493 | like mouse brain protein E46 |
| 132215 | RC_T10132 | AL035703 | Hs 4236 | KIAA0478 gene product |
| 112931 | RC_T15343 | T02966 | Hs.167428 | ESTs |
| 112984 | RC_T23457 | T16971 | Hs.289014 | ESTs, Weakly similar to A43932 mucin 2 precursor, intestinal [H. |
| | | | | sapiens] |
| 112998 | RC_T23555 | H11257 | Hs 22968 | Homo sapiens clone IMAGE: 451939, mRNA sequence |
| 133376 | RC_T23670 | BE618768 | Hs.7232 | acetyl-Coenzyme A carboxylase alpha |
| 113026 | RC_T23948 | AA376654 | Hs 183684 | eukaryotic translation initiation factor 4 gamma, 2 |
| 113070 | RC_T33464 | AB032977 | Hs.6298 | KIAA1151 protein |
| 128970 | RC_T34413 | AI375672 | Hs 165028 | ESTs |
| 113074 | RC_T34611 | AK001335 | Hs.31137 | protein tyrosine phosphatase, receptor type, E |
| 113095 | RC_T40920 | AA828380 | Hs.126733 | ESTs |
| 113179 | RC_T55182 | BE622021 | Hs.152571 | ESTs, Highly similar to IGF-II mRNA-binding protein 2 |
| | | | | [H. sapiens] |
| 113337 | RC_T77453 | T77453 | Hs 302234 | ESTs |
| 113421 | RC_T84039 | AI769400 | Hs.189729 | ESTs |
| 113454 | RC_T86458 | AI022166 | Hs.16188 | ESTs |
| 113481 | RC_T87693 | T87693 | Hs.204327 | EST |
| 131441 | RC_T89350_s | AA302862 | Hs 90063 | neurocalcin delta |
| 113557 | RC_T90945 | H66470 | Hs.16004 | ESTs |
| 113559 | RC_T90987 | T79763 | Hs.14514 | ESTs |
| 113589 | RC_T91863 | AI078554 | Hs.15682 | ESTs |
| 113591 | RC_T91881 | T91881 | Hs.200597 | KIAA0563 gene product |
| 113619 | RC_T93783_s | R08665 | Hs 17244 | hypothetical protein FLJ13605 |
| 113683 | RC_T96687 | AB035335 | Hs 144519 | T-cell leukemia/lymphoma 6 |
| 113692 | RC_T96944 | AL360143 | Hs.17936 | DKFZP434H132 protein |
| 113702 | RC_T97307 | T97307 | | gb: ye53h05.s1 Soares fetal liver spleen 1NFLS Homo sapiens cDNA |
| | | | | clone IMAGE: 121497 3′, mRNA |
| 113717 | RC_T97764 | T99513 | Hs.187447 | ESTs |
| 113824 | RC_W48817 | AI631964 | Hs.34447 | ESTs |
| 113840 | RC_W58343 | R72137 | Hs.7949 | DKFZP586B2420 protein |
| 113844 | RC_W59949 | AI369275 | Hs 243010 | Homo sapiens cDNA FLJ14445 fis, clone HEMBB1001294, highly similar to |
| | | | | GTP-BINDING PROTEIN TC10 |
| 113902 | RC_W74644 | AA340111 | Hs.100009 | acyl-Coenzyme A oxidase 1, palmitoyl |
| 113904 | RC_W74761 | AF125044 | Hs.19196 | ubiquitin-conjugating enzyme HBUCE1 |
| 113905 | RC_W74802 | R81733 | Hs 33106 | ESTs |
| 113931 | RC_W81205 | BE255499 | Hs.3496 | hypothetical protein MGC15749 |
| 113932 | RC_W81237 | AA256444 | Hs.126485 | hypothetical protein FLJ12604; KIAA1692 protein |
| 131965 | RC_W90146_f | W79283 | Hs.35962 | ESTs |
| 114035 | RC_W92798 | W92798 | Hs.269181 | ESTs |
| 114106 | RC_Z38412 | AW602528 | | gb: RC5-BT0562-260100-011-A02 BT0562 Homo sapiens cDNA, mRNA |
| | | | | sequence |
| 133593 | RC_Z38709 | AI416988 | Hs.238272 | inositol 1,4,5-triphosphate receptor, type 2 |
| 114161 | RC_Z38904 | BE548222 | Hs.299883 | hypothetical protein FLJ23399 |
| 424949 | RC_Z39103 | AF052212 | Hs.153934 | core-binding factor, runt domain, alpha subunit 2, translocated |
| | | | | to, 2 |
| 129059 | RC_Z39930_f | AW069534 | Hs.279583 | CGI-81 protein |
| 128937 | RC_Z39939 | AA251380 | Hs.10726 | ESTs, Weakly similar to ALU1_HUMAN ALU SUBFAMILY J SEQUENCE |
| | | | | CONTAMINATION WARNING |
| 130983 | RC_Z40012_i | AI479813 | Hs.278411 | NCK-associated protein 1 |
| 114277 | RC_Z40377_s | AI052229 | Hs.25373 | ESTs, Weakly similar to T20410 hypothetical protein E02A10 2 - |
| | | | | Caenorhabditis elegans [C. elegans] |
| 114304 | RC_Z40820 | AI934204 | Hs.16129 | ESTs |
| 114364 | RC_Z41680 | AL117427 | Hs.172778 | Homo sapiens mRNA; cDNA DKFZp566P013 (from clone DKFZp566P013) |
| 132900 | RC_AA005112 | AA777749 | Hs 5978 | LIM domain only 7 |
| 129034 | RC_AA005432 | AA481157 | Hs.108110 | DKFZP547E2110 protein |
| 131881 | RC_AA010163 | AW361018 | Hs.3383 | upstream regulatory element binding protein 1 |
| 452461 | RC_AA026356 | N78223 | Hs.108106 | transcription factor |
| 114465 | RC_AA026901 | BE621056 | Hs 131731 | hypothetical protein FLJ11099 |
| 131376 | RC_AA036867 | AK001644 | Hs 26156 | hypothetical protein FLJ10782 |
| 101567 | RC_AA044644 | M33552 | Hs.56729 | lysosomal |
| 431555 | RC_AA046426 | AI815470 | Hs.260024 | Cdc42 effector protein 3 |
| 132944 | RC_AA054515 | T96641 | Hs.6127 | Homo sapiens cDNA: FLJ23020 fis, clone LNG00943 |
| 114618 | RC_AA084162 | AW979261 | Hs 291993 | ESTs |
| 130274 | RC_AA085749 | AA128376 | Hs 153884 | ATP binding protein associated with cell differentiation |
| 110330 | RC_AA098874 | AI288666 | Hs.16621 | DKFZP434I116 protein |
| 114648 | RC_AA101056 | AA101056 | | gb: zn25b03.s1 Stratagene neuroepithelium NT2RAMI 937234 |
| | | | | Homo sapiens cDNA clone IMAGE: 548429 3′ |
| 114658 | RC_AA102746 | AA102383 | Hs 249190 | tumor necrosis factor receptor superfamily, member 10a |
| 132456 | RC_AA114250_s | AB011084 | Hs.48924 | KIAA0512 gene product; ALEX2 |
| 131319 | RC_AA126561_s | NM_003155 | Hs 25590 | stanniocalcin 1 |
| 132225 | RC_AA128980_i | AA128980 | | gb: zo09a11.s1 Stratagene neuroepithelium NT2RAMI 937234 |
| | | | | Homo sapiens cDNA clone IMAGE: 567164 3′ |
| 132669 | RC_AA129757 | W38586 | Hs.293981 | guanine nucleotide binding protein (G protein), gamma 3, linked |
| 114709 | RC_AA129921 | AA397651 | Hs.301959 | proline synthetase co-transcribed (bacterial homolog) |
| 131973 | RC_AA133331 | AB018284 | Hs 158688 | KIAA0741 gene product |
| 114750 | RC_AA135958 | AA887211 | Hs.129467 | ESTs |
| 115714 | RC_AA136524_s | T19228 | Hs.172572 | hypothetical protein FLJ20093 |
| 114763 | RC_AA147044 | AA810755 | Hs.88977 | hypothetical protein dJ511E16 2 |
| 114767 | RC_AA148885 | AI859865 | Hs.154443 | minichromosome maintenance deficient (S cerevisiae) 4 |
| 114774 | RC_AA150043 | AV656017 | Hs.184325 | CGI-76 protein |
| 129388 | RC_AA151621 | AA662477 | Hs 110964 | hypothetical protein FLJ23471 |
| 129183 | RC_AA155743 | BE561824 | Hs 273369 | uncharacterized hematopoietic stem/progenitor cells protein |
| | | | | MDS027 |
| 128869 | RC_AA156335 | AA768242 | Hs.80618 | hypothetical protein |
| 130207 | RC_AA156336 | AF044209 | Hs.144904 | nuclear receptor co-repressor 1 |
| 114798 | RC_AA159181 | AA159181 | Hs.54900 | serologically defined colon cancer antigen 1 |
| 114800 | RC_AA159825 | Z19448 | Hs.131887 | ESTs, Weakly similar to T24396 hypothetical protein T03F6.2 - |
| | | | | Caenorhabditis elegans [C. elegans] |
| 114828 | RC_AA234185 | AA252937 | Hs.283522 | Homo sapiens mRNA; cDNA DKFZp434J1912 (from clone DKFZp434J1912) |
| 114846 | RC_AA234929 | BE018682 | Hs.166196 | ATPase, Class I, type 8B, member 1 |
| 114848 | RC_AA234935 | BE614347 | Hs.169615 | hypothetical protein FLJ20989 |
| 114902 | RC_AA236359 | AW275480 | Hs 39504 | hypothetical protein MGC4308 |
| 132271 | RC_AA236466 | AB030034 | Hs.115175 | sterile-alpha motif and leucine zipper containing kinase AZK |
| 114907 | RC_AA236535 | N29390 | Hs.13804 | hypothetical protein dJ462O23 2 |
| 135159 | RC_AA236935_s | U43374 | Hs.95631 | Human normal keratinocyte mRNA |
| 132204 | RC_AA236942 | AA235827 | Hs 42265 | ESTs |
| 114928 | RC_AA237018 | AA237018 | Hs 94869 | ESTs |
| 132481 | RC_AA237025 | W93378 | Hs 49614 | ESTs |
| 114932 | RC_AA242751 | AA971436 | Hs.16218 | KIAA0903 protein |
| 314162 | RC_AA242760 | BE041820 | Hs.38516 | Homo sapiens, clone MGC: 15887, mRNA, complete cds |
| 131006 | RC_AA242763 | AF064104 | Hs.22116 | CDC14 (cell division cycle 14, S. cerevisiae) homolog B |
| 114935 | RC_AA242809 | H23329 | Hs.290880 | ESTs, Weakly similar to ALU1_HUMAN ALU SUBFAMILY J SEQUENCE |
| | | | | CONTAMINATION |
| 132454 | RC_AA243133 | BE296227 | Hs.250822 | serine/threonine kinase 15 |
| 437754 | RC_AA243495 | R60366 | Hs.5822 | Homo sapiens cDNA: FLJ22120 fis, clone HEP18874 |
| 114957 | RC_AA243706 | AW170425 | Hs.87680 | ESTs |
| 114974 | RC_AA250848 | AW966931 | Hs.179662 | nucleosome assembly protein 1-like 1 |
| 114977 | RC_AA250868 | AW296978 | Hs.87787 | ESTs |
| 114995 | RC_AA251152 | AA769266 | Hs.193657 | ESTs |
| 115005 | RC_AA251544_s | AI760825 | Hs.111339 | ESTs |
| 417177 | RC_AA251792 | NM_004458 | Hs.81452 | fatty-acid-Coenzyme A ligase, long-chain 4 |
| 131889 | RC_AA252063 | NM_002589 | Hs.34073 | BH-protocadherin (brain-heart) |
| 115026 | RC_AA252144 | AA251972 | Hs.188718 | ESTs |
| 115045 | RC_AA252524 | AW014549 | Hs.58373 | ESTs |
| 115068 | RC_AA253461 | AW512260 | Hs.87767 | ESTs |
| 133138 | RC_AA255522 | AV657594 | Hs.181161 | Homo sapiens cDNA FLJ14643 fis, clone NT2RP2001597, weakly |
| | | | | similar to RYANODINE RECEPTOR, |
| 115114 | RC_AA256468 | AA527548 | Hs.7527 | small fragment nuclease |
| 129584 | RC_AA256528 | AV656017 | Hs.184325 | CGI-76 protein |
| 115137 | RC_AA257976 | AW968304 | Hs 56156 | ESTs |
| 134312 | RC_AA258296 | AB011151 | Hs.334659 | hypothetical protein MGC14139 |
| 115166 | RC_AA258409 | AF095727 | Hs 287832 | myelin protein zero-like 1 |
| 115167 | RC_AA258421 | AA749209 | Hs.43728 | hypothetical protein |
| 129807 | RC_AA262077 | Y11192 | Hs.5299 | aldehyde dehydrogenase 5 family, member A1 (succinate- |
| | | | | semialdehyde dehydrogenase) |
| 115239 | RC_AA278650 | BE251328 | Hs.73291 | hypothetical protein FLJ10881 |
| 115243 | RC_AA278766 | AA806600 | Hs.116665 | KIAA1842 protein |
| 100850 | RC_AA279667_s | AA836472 | Hs.297939 | cathepsin B |
| 126884 | RC_AA280791 | U49436 | Hs 286236 | KIAA1856 protein |
| 115322 | RC_AA280819 | L08895 | Hs.78995 | MADS box transcription enhancer factor 2, polypeptide C (myocyte |
| | | | | enhancer factor 2C) |
| 133626 | RC_AA280828 | AW836130 | Hs.75277 | hypothetical protein FLJ13910 |
| 115372 | RC_AA282195 | AW014385 | Hs 88678 | ESTs, Weakly similar to Unknown [H. sapiens] |
| 132825 | RC_AA283127_s | U82671 | Hs.57698 | Empirically selected from AFFX single probeset |
| 130269 | RC_AA284694 | F05422 | Hs 168352 | nucleoporin-like protein 1 |
| 129192 | RC_AA291137 | AA286914 | Hs.183299 | ESTs |
| 452598 | RC_AA291708 | AI831594 | Hs.68647 | ESTs, Weakly similar to ALU7_HUMAN ALU SUBFAMILY SQ SEQUENCE |
| | | | | CONTAMINATION WARNING |
| 132131 | RC_AA293495 | AF069291 | Hs 40539 | chromosome 8 open reading frame 1 |
| 115536 | RC_AA347193 | AK001468 | Hs.62180 | anillin (Drosophila Scraps homolog), actin binding protein |
| 132411 | RC_AA398474_s | AA059412 | Hs.47986 | hypothetical protein MGC10940 |
| 115575 | RC_AA398512 | AA393254 | Hs.43619 | ESTs |
| 115601 | RC_AA400277 | AA148984 | Hs.48849 | ESTs, Weakly similar to ALU4_HUMAN ALU SUBFAMILY SB2 SEQUENCE |
| | | | | CONTAMINATION WARNING |
| 103928 | RC_AA400896 | D14540 | Hs.199160 | myeloid/lymphoid or mixed-lineage leukemia (trithorax (Drosophila) |
| | | | | homolog) |
| 125819 | RC_AA404494 | AA044840 | Hs 251871 | CTP synthase |
| 115683 | RC_AA410345 | AF255910 | Hs 54650 | junctional adhesion molecule 2 |
| 115715 | RC_AA416733 | BE395161 | Hs 1390 | proteasome (prosome, macropain) subunit, beta type, 2 |
| 132952 | RC_AA425154 | AI658580 | Hs.61426 | Homo sapiens mesenchymal stem cell protein DSC96 mRNA, partial |
| | | | | cds |
| 115819 | RC_AA426573 | AA486620 | Hs 41135 | endomucin-2 |
| 132525 | RC_AA431418 | AW292809 | Hs.50727 | N-acetylglucosaminidase, alpha-(Sanfilippo disease IIIB) |
| 115895 | RC_AA436182 | AB033035 | Hs.51965 | KIAA1209 protein |
| 132333 | RC_AA437099 | AA192669 | Hs.45032 | ESTs |
| 115962 | RC_AA446585 | AI636361 | Hs.179520 | hypothetical protein MGC10702 |
| 115967 | RC_AA446887 | AI745379 | Hs.42911 | ESTs |
| 115974 | RC_AA447224 | BE513442 | Hs 238944 | hypothetical protein FLJ10631 |
| 115985 | RC_AA447709 | AA447709 | Hs.268115 | ESTs, Weakly similar to T08599 probable transcription factor |
| | | | | CA150 [H. sapiens] |
| 129254 | RC_AA453624 | AA252468 | Hs 1098 | DKFZp434J1813 protein |
| 133071 | RC_AA455044 | BE384932 | Hs.64313 | ESTs, Weakly similar to AF257182 1 G-protein-coupled receptor 48 |
| | | | | [H. sapiens] |
| 116095 | RC_AA456045 | AA043429 | Hs.62618 | ESTs |
| 122691 | RC_AA460454_s | R19768 | Hs.172788 | ALEX3 protein |
| 116210 | RC_AA476494 | BE622792 | Hs.172788 | ALEX3 protein |
| 116213 | RC_AA476738 | AA292105 | Hs 326740 | hypothetical protein MGC10947 |
| 134585 | RC_AA481422 | D14041 | Hs.278573 | H-2K binding factor-2 |
| 134790 | RC_AA482269 | BE002798 | Hs.287850 | integral membrane protein 1 |
| 116265 | RC_AA482595 | BE297412 | Hs.55189 | hypothetical protein |
| 129334 | RC_AA485084_s | AW157022 | Hs.4947 | hypothetical protein FLJ22584 |
| 116274 | RC_AA485431_s | AI129767 | Hs.182874 | guanine nucleotide binding protein (G protein) alpha 12 |
| 303150 | RC_AA489057 | AA887146 | Hs.8217 | stromal antigen 2 |
| 129945 | RC_AA489638 | BE514376 | Hs.165998 | PAI-1 mRNA-binding protein |
| 116331 | RC_AA491000 | N41300 | Hs.71616 | Homo sapiens mRNA; cDNA DKFZp586N1720 (from clone DKFZp586N1720) |
| 116333 | RC_AA491250 | AF155827 | Hs.203963 | hypothetical protein FLJ10339 |
| 132994 | RC_AA505133 | AA112748 | Hs.279905 | clone HQ0310 PRO0310p1 |
| 134577 | RC_AA598447 | BE244323 | Hs 85951 | exportin, tRNA (nuclear export receptor for tRNAs) |
| 116391 | RC_AA599243 | T86558 | Hs 75113 | general transcription factor IIIA |
| 116394 | RC_AA599574_i | NM_006033 | Hs.65370 | lipase, endothelial |
| 134531 | RC_AA600153 | AI742845 | Hs.110713 | DEK oncogene (DNA binding) |
| 116417 | RC_AA609309 | AW499664 | Hs.12484 | Human clone 23826 mRNA sequence |
| 116429 | RC_AA609710 | AF191018 | Hs.279923 | putative nucleotide binding protein, estradiol-induced |
| 116439 | RC_AA610068 | AA251594 | Hs.43913 | PIBF1 gene product |
| 116459 | RC_AA621399 | R80137 | Hs.302738 | Homo sapiens cDNA. FLJ21425 fis, clone COL04162 |
| 427505 | RC_AA621752 | AA361562 | Hs.178761 | 26S proteasome-associated pad1 homolog |
| 132699 | RC_C21523 | AW449822 | Hs.55200 | ESTs |
| 116541 | RC_D12160 | D12160 | Hs.249212 | polymerase (RNA) III (DNA directed) (155 kD) |
| 132557 | RC_D19708 | AA114926 | Hs.5122 | ESTs |
| 112259 | RC_D25801 | AA337548 | Hs 333402 | hypothetical protein MGC12760 |
| 116571 | RC_D45652 | D45652 | | gb: HUMGS02848 Human adult lung 3′ directed Mbol cDNA |
| | | | | Homo sapiens cDNA 3′, mRNA sequence. |
| 129815 | RC_D60208_f | BE565817 | Hs.26498 | hypothetical protein FLJ21657 |
| 421919 | RC_D80504_s | AJ224901 | Hs.109526 | zinc finger protein 198 |
| 116643 | RC_F03010 | AI367044 | Hs.153638 | myeloid/lymphoid or mixed-lineage leukemia 2 |
| 116661 | RC_F04247 | R61504 | | gb: yh16a03.s1 Soares infant brain 1NIB Homo sapiens cDNA |
| | | | | clone 3′ similar to contains Alu repetitive |
| 116715 | RC_F10966 | AL117440 | Hs 170263 | tumor protein p53-binding protein, 1 |
| 116729 | RC_F13700 | BE549407 | Hs 115823 | ribonuclease P, 40 kD subunit |
| 318709 | RC_H05063 | R52576 | Hs.285280 | Homo sapiens cDNA: FLJ22096 fis, clone HEP16953 |
| 134760 | RC_H16758 | NM_000121 | Hs.89548 | erythropoietin receptor |
| 116773 | RC_H17315_s | AI823410 | Hs.169149 | karyopherin alpha 1 (importin alpha 5) |
| 106425 | RC_H22556 | H24201 | Hs.247423 | adducin 2 (beta) |
| 116780 | RC_H22566 | H22566 | Hs.30098 | ESTs |
| 131978 | RC_H48459_s | AA355925 | Hs.36232 | KIAA0186 gene product |
| 116819 | RC_H53073 | H53073 | Hs.93698 | EST |
| 111428 | RC_H56559_s | AL031428 | Hs.174174 | KIAA0601 protein |
| 133175 | RC_H57957_s | AW955632 | Hs.66666 | ESTs, Weakly similar to S19560 proline-rich protein MP4 - mouse |
| | | | | [M. musculus] |
| 116844 | RC_H64938_s | H64938 | Hs.337434 | ESTs, Weakly similar to A46010 X-linked retinopathy protein |
| | | | | [H. sapiens] |
| 116845 | RC_H64973 | AA649530 | | gb: ns44f05.s1 NCI_CGAP_Alv1 Homo sapiens cDNA clone, mRNA |
| | | | | sequence |
| 116892 | RC_H69535 | AI573283 | Hs.38458 | ESTs |
| 116925 | RC_H73110 | H73110 | Hs 260603 | ESTs, Moderately similar to A47582 B-cell growth factor precursor |
| | | | | [H. sapiens] |
| 116981 | RC_H81783 | N29218 | Hs.40290 | ESTs |
| 131768 | RC_H86259 | AC005757 | Hs.31809 | hypothetical protein |
| 117031 | RC_H88353 | H88353 | | gb: yw21a02.s1 Morton Fetal Cochlea Homo sapiens cDNA clone |
| | | | | IMAGE: 252842 3′ similar to contains L1 |
| 117034 | RC_H88639 | U72209 | Hs.180324 | YY1-associated factor 2 |
| 132542 | RC_H88675 | AL137751 | Hs.263671 | Homo sapiens mRNA; cDNA DKFZp434I0812 (from clone DKFZp434I0812); |
| | | | | partial cds |
| 134403 | RC_H93708_s | AA334551 | Hs 82767 | sperm specific antigen 2 |
| 117280 | RC_N22107 | M18217 | Hs.172129 | Homo sapiens cDNA: FLJ21409 fis, clone COL03924 |
| 117344 | RC_N24046 | R19085 | Hs.210706 | Homo sapiens cDNA FLJ13182 fis, clone NT2RP3004070 |
| 117422 | RC_N27028 | AI355562 | Hs 43880 | ESTs, Weakly similar to A46010 X-linked retinopathy protein |
| | | | | [H sapiens] |
| 117475 | RC_N30205 | N30205 | Hs.93740 | ESTs, Weakly similar to I38022 hypothetical protein |
| | | | | [H. sapiens] |
| 117487 | RC_N30621 | N30621 | Hs.44203 | ESTs |
| 130207 | RC_N33258 | AF044209 | Hs.144904 | nuclear receptor co-repressor 1 |
| 117549 | RC_N33390 | N33390 | Hs 44483 | EST |
| 117683 | RC_N40180 | N40180 | | gb: yy44d02.s1 Soares_multiple_sclerosis_2NbHMSP Homo sapiens cDNA |
| | | | | clone IMAGE: 276387 3′ similar to |
| 117710 | RC_N45198 | N45198 | Hs.47248 | ESTs, Highly similar to similar to Cdc14B1 phosphatase |
| | | | | [H sapiens] |
| 104514 | RC_N45979_s | AF164622 | Hs.182982 | golgin-67 |
| 117791 | RC_N48325 | N48325 | Hs.93956 | EST |
| 117822 | RC_N48913 | AA706282 | Hs 93963 | ESTs |
| 129647 | RC_N49394 | AB018259 | Hs.118140 | KIAA0716 gene product |
| 117895 | RC_N50656 | AW450348 | Hs.93996 | ESTs, Highly similar to SORL_HUMAN SORTILIN-RELATED RECEPTOR |
| | | | | PRECURSOR [H. sapiens] |
| 131557 | RC_N50721 | AA317439 | Hs 28707 | signal sequence receptor, gamma (translocon-associated protein |
| | | | | gamma) |
| 133057 | RC_N53143 | AA465131 | Hs.64001 | Homo sapiens clone 25218 mRNA sequence |
| 118103 | RC_N55326 | AA401733 | Hs.184134 | ESTs |
| 118111 | RC_N55493 | N55493 | | gb: yv50c02.s1 Soares fetal liver spleen 1NFLS Homo sapiens cDNA |
| | | | | clone IMAGE: 246146 3′, mRNA |
| 118129 | RC_N57493 | N57493 | | gb: yy54c08.s1 Soares_multiple_sclerosis_2NbHMSP Homo sapiens cDNA |
| | | | | clone IMAGE: 277358 3′, mRNA |
| 118278 | RC_N62955 | N62955 | Hs 316433 | Homo sapiens cDNA FLJ11375 fis, clone HEMBA1000411, weakly similar |
| | | | | to ANKYRIN |
| 118329 | RC_N63520 | N63520 | | gb: yy62f01.s1 Soares_multiple_sclerosis_2NbHMSP Homo sapiens cDNA |
| | | | | clone IMAGE: 278137 3′, mRNA |
| 118336 | RC_N63604 | BE327311 | Hs.47166 | HT021 |
| 132457 | RC_N64166 | AB017365 | Hs.173859 | frizzled (Drosophila) homolog 7 |
| 118363 | RC_N64168 | AI183838 | Hs 48938 | hypothetical protein FLJ21802 |
| 118364 | RC_N64191 | N46114 | Hs.29169 | hypothetical protein FLJ22623 |
| 118475 | RC_N66845 | N66845 | | gb: za46c11.s1 Soares fetal liver spleen 1NFLS Homo sapiens cDNA |
| | | | | clone IMAGE 295604 3′ similar to |
| 118491 | RC_N67135 | AV647908 | Hs.90424 | Homo sapiens cDNA: FLJ23285 fis, clone HEP09071 |
| 118500 | RC_N67295 | W32889 | Hs.154329 | ESTs |
| 101663 | RC_N68399 | NM_003528 | Hs.2178 | H2B histone family, member Q |
| 118584 | RC_N68963 | AW136928 | | gb: UI-H-BI1-adp-d-08-0-UI.s1 NCI_CGAP_Sub3 Homo sapiens cDNA clone |
| | | | | 3′, mRNA sequence |
| 421983 | RC_N69331 | AI252640 | Hs.110364 | peptidylprolyl isomerase C (cyclophilin C) |
| 118661 | RC_N70777 | AL137554 | Hs 49927 | protein kinase NYD-SP15 |
| 118684 | RC_N71364_s | N71313 | Hs.163986 | Homo sapiens cDNA: FLJ22765 fis, clone KAIA1180 |
| 118689 | RC_N71545_s | AW390601 | Hs.184544 | Homo sapiens, clone IMAGE: 3355383, mRNA, partial cds |
| 118690 | RC_N71571 | N71571 | Hs.269142 | ESTs |
| 118766 | RC_N74456 | N74456 | Hs 50499 | EST |
| 118793 | RC_N75594 | N75594 | Hs.285921 | ESTs, Moderately similar to T47135 hypothetical protein |
| | | | | DKFZp761L0812.1 [H. sapiens] |
| 118817 | RC_N79035 | AI668658 | Hs.50797 | ESTs |
| 118844 | RC_N80279 | AL035364 | Hs.50891 | hypothetical protein |
| 118919 | RC_N91797 | AW452696 | Hs 130760 | myosin phosphatase, target subunit 2 |
| 129558 | RC_N92454 | AW580922 | Hs 180446 | karyopherin (importin) beta 1 |
| 132692 | RC_N94581 | AW191962 | Hs 249239 | collagen, type VIII, alpha 2 |
| 118996 | RC_N94746 | N94746 | Hs.274248 | hypothetical protein FLJ20758 |
| 119021 | RC_N98238 | N98238 | Hs.55185 | ESTs |
| 119039 | RC_R02384 | AI160570 | Hs.252097 | pregnancy specific beta-1-glycoprotein 6 |
| 119063 | RC_R16833 | R16833 | Hs.53106 | ESTs, Moderately similar to ALU1_HUMAN ALU SUBFAMILY J SEQUENCE |
| | | | | CONTAMINATION WARNING |
| 118523 | RC_R41828_s | Y07759 | Hs.170157 | myosin VA (heavy polypeptide 12, myoxin) |
| 119111 | RC_R43203 | T02865 | Hs.328321 | EST |
| 133970 | RC_R46395 | AA214228 | Hs.127751 | hypothetical protein |
| 119146 | RC_R58863 | R58863 | Hs.91815 | ESTs |
| 120296 | RC_R78248 | AW995911 | Hs 299883 | hypothetical protein FLJ23399 |
| 119239 | RC_T11483 | T11483 | | gb: CHR90049 Chromosome 9 exon Homo sapiens cDNA clone 111-1 |
| | | | | 5′ and 3′, mRNA sequence. |
| 119281 | RC_T16896 | AI692322 | Hs 65373 | ESTs, Weakly similar to T02345 hypothetical protein KIAA0324 |
| | | | | [H. sapiens] |
| 119298 | RC_T23820 | NM_001241 | Hs.155478 | cyclin T2 |
| 126502 | RC_T30222 | T10077 | Hs.13453 | hypothetical protein FLJ14753 |
| 135073 | RC_W15275_s | W55956 | Hs.94030 | Homo sapiens mRNA; cDNA DKFZp586E1624 (from clone DKFZp586E1624) |
| 119558 | RC_W38194 | W38194 | | Empirically selected from AFFX single probeset |
| 132736 | RC_W42414_s | AW081883 | Hs 288261 | Homo sapiens cDNA: FLJ23037 fis, clone LNG02036, highly similar |
| | | | | to HSU68019 Homo sapiens mad protein |
| 132173 | RC_W46577_s | X89426 | Hs.41716 | endothelial cell-specific molecule 1 |
| 134873 | RC_W49632_s | AA884471 | Hs 90449 | Human clone 23908 mRNA sequence |
| 119650 | RC_W57613 | R82342 | Hs.79856 | ESTs, Weakly similar to S65657 alpha-1C-adrenergic receptor |
| | | | | splice form 2 [H. sapiens] |
| 119654 | RC_W57759 | W57759 | | gb: zd20g11.s1 Soares_fetal_heart_NbHH19W Homo sapiens cDNA clone |
| | | | | IMAGE: 341252 3′ similar to |
| 119683 | RC_W61118 | W65379 | Hs.57835 | ESTs |
| 119694 | RC_W65344 | AA041350 | Hs.57847 | ESTs, Moderately similar to ICE4_HUMAN CASPASE-4 PRECURSOR |
| | | | | [H. sapiens] |
| 119718 | RC_W69216 | W69216 | Hs.92848 | ESTs |
| 133010 | RC_W69379 | AI287518 | Hs.62669 | Homo sapiens mRNA; cDNA DKFZp586D0923 (from clone DKFZp586D0923) |
| 119938 | RC_W86728 | AW014862 | Hs.58885 | ESTs |
| 120128 | RC_Z38499 | BE379320 | Hs.91448 | MKP-1 like protein tyrosine phosphatase |
| 120130 | RC_Z38630 | AA045767 | Hs.5300 | bladder cancer associated protein |
| 120148 | RC_Z39494 | F02806 | Hs.65765 | ESTs |
| 120155 | RC_Z39623 | Z39623 | Hs.65783 | ESTs |
| 131486 | RC_Z40071_s | F06972 | Hs.27372 | BMX non-receptor tyrosine kinase |
| 120183 | RC_Z40174 | AW082866 | Hs 65882 | ESTs |
| 120184 | RC_Z40182 | Z40182 | Hs.65885 | EST |
| 120211 | RC_Z40904 | Z40904 | Hs.66012 | EST |
| 120245 | RC_AA166965 | AW959615 | Hs.111045 | ESTs |
| 120247 | RC_AA167500 | AA167500 | Hs.103939 | EST |
| 120254 | RC_AA169599_s | W90403 | Hs.111054 | ESTs |
| 120259 | RC_AA171724 | AW014786 | Hs.192742 | hypothetical protein FLJ12785 |
| 120260 | RC_AA171739 | AK000061 | Hs.101590 | hypothetical protein |
| 120275 | RC_AA177105 | AA177105 | Hs 78457 | solute carrier family 25 (mitochondrial carrier; ornithine |
| | | | | transporter) member 15 |
| 120284 | RC_AA182626 | AA179656 | | gb: zp54e11.s1 Stratagene NT2 neuronal precursor 937230 |
| | | | | Homo sapiens cDNA clone 3′ similar to contains |
| 114056 | RC_AA186324 | AA188175 | Hs 82506 | KIAA1254 protein |
| 129507 | RC_AA192099 | AJ236885 | Hs.112180 | zinc finger protein 148 (pHZ-52) |
| 120302 | RC_AA192173 | AA837098 | Hs.269933 | ESTs |
| 120303 | RC_AA192415 | AI216292 | Hs 96184 | ESTs |
| 120305 | RC_AA192553 | AW295096 | Hs.101337 | uncoupling protein 3 (mitochondrial, proton carrier) |
| 120319 | RC_AA194851 | T57776 | Hs 191094 | ESTs |
| 133389 | RC_AA195520_s | AA195764 | Hs.72639 | ESTs |
| 120326 | RC_AA196300 | AA196300 | Hs 21145 | hypothetical protein RG083M05.2 |
| 134272 | RC_AA196517 | X76040 | Hs.278614 | protease, serine, 15 |
| 133145 | RC_AA196549 | H94227 | Hs.6592 | Homo sapiens, clone IMAGE: 2961368, mRNA, partial cds |
| 120327 | RC_AA196721 | AK000292 | Hs.278732 | hypothetical protein FLJ20285 |
| 106686 | RC_AA196729_i | N66397 | Hs.334825 | Homo sapiens cDNA FLJ14752 fis, clone NT2RP3003071 |
| 120328 | RC_AA196979 | AA923278 | Hs.290905 | ESTs, Weakly similar to protease [H. sapiens] |
| 120340 | RC_AA206828 | AA206828 | | gb: zq80b08.s1 Stratagene hNT neuron (937233) Homo sapiens cDNA |
| | | | | clone IMAGE: 647895 3′ similar to |
| 134292 | RC_AA207123 | AI906291 | Hs.81234 | immunoglobulin superfamily, member 3 |
| 131522 | RC_AA214539_i | AI380040 | Hs.239489 | TIA1 cytotoxic granule-associated RNA-binding protein |
| 129051 | RC_AA226914_s | AA227068 | Hs.108301 | nuclear receptor subfamily 2, group C, member 1 |
| 120375 | RC_AA227260 | AF028706 | Hs.111227 | Zic family member 3 (odd-paired Drosophila homolog, heterotaxy 1) |
| 120376 | RC_AA227469 | AA227469 | | gb: zr18a07.s1 Stratagene NT2 neuronal precursor 937230 |
| | | | | Homo sapiens cDNA clone IMAGE: 663732 3′, mRNA sequence |
| 120390 | RC_AA233122 | AA837093 | Hs.111460 | calcium/calmodulin-dependent protein kinase (CaM kinase) II delta |
| 303876 | RC_AA233334_s | U64820 | Hs.66521 | Machado-Joseph disease (spinocerebellar ataxia 3, |
| | | | | olivopontocerebellar ataxia 3, autosomal dominant, ataxin 3) |
| 132038 | RC_AA233347 | AI825842 | Hs.3776 | zinc finger protein 216 |
| 104463 | RC_AA233519 | T85825 | Hs.246885 | hypothetical protein FLJ20783 |
| 125750 | RC_AA233714 | AA018515 | Hs.264482 | Homo sapiens mRNA; cDNA DKFZp761A0411 (from clone DKFZp761A0411) |
| 120396 | RC_AA233796 | AA134006 | Hs 79306 | eukaryotic translation initiation factor 4E |
| 120409 | RC_AA235050_f | AA235050 | | gb: zs38e04.s1 Soares_NhHMPu_S1 Homo sapiens cDNA clone |
| | | | | IMAGE: 687486 3′ similar to gb: L07077 |
| 120414 | RC_AA235704 | AW137156 | Hs.181202 | hypothetical protein FLJ10038 |
| 120420 | RC_AA236031 | AI128114 | Hs.112885 | spinal cord-derived growth factor-B |
| 120422 | RC_AA236352 | AL133097 | Hs 301717 | hypothetical protein DKFZp434N1928 |
| 132221 | RC_AA236390_s | W94915 | Hs.42419 | ESTs |
| 120423 | RC_AA236453 | AA236453 | Hs.18978 | Homo sapiens cDNA: FLJ22822 fis, clone KAIA3968 |
| 120435 | RC_AA243370 | AA243370 | Hs.96450 | EST |
| 120453 | RC_AA250947 | AA250947 | Hs.170263 | tumor protein p53-binding protein, 1 |
| 120455 | RC_AA251083 | AA251720 | Hs.104347 | ESTs, Weakly similar to ALUC_HUMAN !!!! ALU CLASS C |
| | | | | WARNING ENTRY !!! [H. sapiens] |
| 120456 | RC_AA251113 | AA488750 | Hs.88414 | BTB and CNC homology 1, basic leucine zipper transcription |
| | | | | factor 2 |
| 120473 | RC_AA251973 | AA251973 | Hs.269988 | ESTs |
| 128922 | RC_AA252023 | AI244901 | Hs.9589 | ubiquilin 1 |
| 120477 | RC_AA252414 | AA252414 | Hs.43141 | DKFZP727C091 protein |
| 120479 | RC_AA252650 | AF006689 | Hs.110299 | mitogen-activated protein kinase kinase 7 |
| 120488 | RC_AA255523 | AW952916 | Hs.63510 | KIAA0141 gene product |
| 120510 | RC_AA258128 | AI796395 | Hs.111377 | ESTs |
| 120527 | RC_AA262105 | AA262105 | Hs.4094 | Homo sapiens cDNA FLJ14208 fis, clone NT2RP3003264 |
| 120528 | RC_AA262107 | AI923511 | Hs.104413 | ESTs |
| 120529 | RC_AA262235 | AI434823 | Hs.104415 | ESTs |
| 120541 | RC_AA278298 | W07318 | Hs 240 | M-phase phosphoprotein 1 |
| 131445 | RC_AA278529_i | NM_014264 | Hs.172052 | serine/threonine kinase 18 |
| 120544 | RC_AA278721 | BE548277 | Hs.103104 | ESTs |
| 120562 | RC_AA280036 | BE244580 | Hs 302267 | hypothetical protein FLJ10330 |
| 120569 | RC_AA280648 | AA807544 | Hs.24970 | ESTs, Weakly similar to B34323 GTP-binding protein Rab2 |
| | | | | [H sapiens] |
| 120571 | RC_AA280738 | AB037744 | Hs.34892 | KIAA1323 protein |
| 120572 | RC_AA280794 | H39599 | Hs.294008 | ESTs |
| 129434 | RC_AA280837 | AW967495 | Hs.186644 | ESTs |
| 130529 | RC_AA280886 | AA178953 | | gb: zp39e03.s1 Stratagene muscle 937209 Homo sapiens cDNA clone |
| | | | | 3′ similar to contains Alu repetitive |
| 120575 | RC_AA280934 | AW978022 | Hs.238911 | hypothetical protein DKFZp762E1511, KIAA1816 protein |
| 132635 | RC_AA281535 | AB020686 | Hs.54037 | ectonucleotide pyrophosphatase/phosphodiesterase 4 (putative |
| | | | | function) |
| 120591 | RC_AA281797_s | AF078847 | Hs.191356 | general transcription factor IIH, polypeptide 2 (44 kD subunit) |
| 120593 | RC_AA282047 | AA748355 | Hs.193522 | ESTs |
| 430275 | RC_AA283002 | Z11773 | Hs.237786 | zinc finger protein 187 |
| 117729 | RC_AA283709 | AA306166 | Hs.7145 | calpain 7 |
| 120609 | RC_AA283902 | AW978721 | Hs.266076 | ESTs, Weakly similar to A46010 X-linked retinopathy protein |
| | | | | [H. sapiens] |
| 132754 | RC_AA284108 | AI752244 | Hs.75309 | eukaryotic translation elongation factor 2 |
| 130315 | RC_AA284109 | AI241084 | Hs.154353 | nonselective sodium potassium/proton exchanger |
| 132614 | RC_AA284371 | AA284371 | Hs.118064 | similar to rat nuclear ubiquitous casein kinase 2 |
| 447503 | RC_AA284744_f | AA115496 | Hs 336898 | Homo sapiens, Similar to RIKEN cDNA 1810038N03 gene, clone |
| | | | | MGC: 9890, mRNA, complete cds |
| 135376 | RC_AA284784 | BE617856 | Hs.99756 | mitochondrial ribosome recycling factor |
| 120621 | RC_AA284840 | AW961294 | Hs.143818 | hypothetical protein FLJ23459 |
| 107868 | RC_AA286844 | AA286844 | Hs.61260 | hypothetical protein FLJ13164 |
| 129868 | RC_AA287032 | AW172431 | Hs.13012 | ESTs |
| 120644 | RC_AA287038 | AI869129 | Hs 96616 | ESTs |
| 120660 | RC_AA287546 | AA286785 | Hs.99677 | ESTs |
| 135370 | RC_AA287553_s | BE622187 | Hs 99670 | ESTs, Weakly similar to I38022 hypothetical protein |
| | | | | [H. sapiens] |
| 120661 | RC_AA287556 | AA287556 | Hs 263412 | ESTs, Weakly similar to ALUB_HUMAN !!!! ALU CLASS B |
| | | | | WARNING ENTRY !!! [H sapiens] |
| 129116 | RC_AA287564 | AB019494 | Hs 225767 | IDN3 protein |
| 131567 | RC_AA291015_s | AF015592 | Hs.28853 | CDC7 (cell division cycle 7, S. cerevisiae, homolog)-like 1 |
| 120699 | RC_AA291716 | AI683243 | Hs 97258 | ESTs, Moderately similar to S29539 ribosomal protein L13a, |
| | | | | cytosolic [H. sapiens] |
| 100690 | RC_AA291749_s | AA383256 | Hs.1657 | estrogen receptor 1 |
| 120726 | RC_AA293656 | AA293655 | Hs.97293 | ESTs |
| 120737 | RC_AA302430 | AL049176 | Hs.82223 | chordin-like |
| 120745 | RC_AA302809 | AA302809 | | gb: EST10426 Adipose tissue, white I Homo sapiens cDNA 3′ end, |
| | | | | mRNA sequence. |
| 135192 | RC_AA302820_s | U83993 | Hs.321709 | purinergic receptor P2X, ligand-gated ion channel, 4 |
| 120750 | RC_AA310499 | AI191410 | Hs 96693 | ESTs, Moderately similar to 2109260A B cell growth factor |
| | | | | [H. sapiens] |
| 120761 | RC_AA321890 | AA321890 | Hs.1265 | branched chain keto acid dehydrogenase E1, beta polypeptide (maple |
| | | | | syrup urine disease) |
| 120768 | RC_AA340589 | AA340589 | Hs 104560 | EST |
| 120769 | RC_AA340622 | AI769467 | Hs 96769 | ESTs |
| 135232 | RC_AA342457_i | AL038812 | Hs.96800 | ESTs, Moderately similar to ALU7_HUMAN ALU SUBFAMILY SQ SEQUENCE |
| | | | | CONTAMINATION |
| 133439 | RC_AA342828_s | Z23091 | Hs.73734 | glycoprotein V (platelet) |
| 120793 | RC_AA342864 | AA342864 | Hs.96812 | ESTs |
| 120796 | RC_AA342973 | AI247356 | Hs.96820 | ESTs |
| 120809 | RC_AA346495 | AA346495 | | gb: EST52657 Fetal heart II Homo sapiens cDNA 3′ end similar to |
| | | | | EST containing O family repeat, mRNA sequence. |
| 132459 | RC_AA347573 | AL120071 | Hs.48998 | fibronectin leucine rich transmembrane protein 2 |
| 120825 | RC_AA347614 | AI280215 | Hs.96885 | ESTs |
| 120827 | RC_AA347717 | AA382525 | Hs.132967 | Human EST clone 122887 mariner transposon Hsmar1 sequence |
| 120839 | RC_AA348913 | AA348913 | | gb: EST55442 Infant adrenal gland II Homo sapiens cDNA 3′ end |
| | | | | similar to EST containing Alu repeat, mRNA sequence. |
| 120850 | RC_AA349647 | AA349647 | Hs 96927 | Homo sapiens cDNA FLJ12573 fis, clone NT2RM4000979 |
| 120852 | RC_AA349773 | AA349773 | Hs.191564 | ESTs |
| 128852 | RC_AA350541_s | R40622 | Hs 106601 | ESTs |
| 135240 | RC_AA357159_i | AA357159 | Hs 96986 | EST |
| 120870 | RC_AA357172_i | AA357172 | Hs.292581 | ESTs, Moderately similar to ALU1_HUMAN ALU SUBFAMILY J SEQUENCE |
| | | | | CONTAMINATION WARNING |
| 134637 | RC_AA369856_s | U87309 | Hs.180941 | vacuolar protein sorting 41 (yeast homolog) |
| 120894 | RC_AA370132 | AA370132 | Hs.97063 | ESTs |
| 131854 | RC_AA370472_s | AF229839 | Hs 173202 | I-kappa-B-interacting Ras-like protein 1 |
| 120897 | RC_AA370867 | AA370867 | Hs.97079 | ESTs, Moderately similar to AF174605 1 F-box protein Fbx25 |
| | | | | [H. sapiens] |
| 120915 | RC_AA377296 | AL135556 | Hs.97104 | ESTs |
| 120935 | RC_AA383902 | AL048409 | Hs 97177 | ESTs, Weakly similar to ALU1_HUMAN ALU SUBFAMILY J SEQUENCE |
| | | | | CONTAMINATION WARNING |
| 120936 | RC_AA385934 | AA385934 | Hs.97184 | EST, Highly similar to (defline not available 7499603) |
| | | | | [C. elegans] |
| 120937 | RC_AA386255 | AA386255 | Hs.97186 | EST |
| 120938 | RC_AA386260 | AA386260 | Hs 104632 | EST |
| 129722 | RC_AA386266 | R20855 | Hs.5422 | glycoprotein M6B |
| 120960 | RC_AA398014 | AA398014 | Hs.104684 | EST |
| 120985 | RC_AA398222 | AI219896 | Hs.97592 | ESTs |
| 120988 | RC_AA398235 | AA398235 | Hs.97631 | ESTs |
| 121008 | RC_AA398348 | AA398348 | Hs.301720 | Human DNA sequence from clone RP11-251J8 on chromosome 13 Contains |
| | | | | ESTs, STSs, GSSs and a CpG |
| 121029 | RC_AA398482 | AA398482 | Hs.97641 | EST |
| 121032 | RC_AA398504 | AA393037 | Hs.161798 | ESTs |
| 121033 | RC_AA398505 | AA398505 | Hs 97360 | ESTs |
| 121034 | RC_AA398507 | AL389951 | Hs 271623 | nucleoporin 50 kD |
| 121035 | RC_AA398523 | AA398523 | Hs.210579 | ESTs |
| 121058 | RC_AA398625 | AA398625 | Hs.97391 | ESTs |
| 121060 | RC_AA398632 | AA398632 | Hs.97395 | ESTs |
| 121061 | RC_AA398633 | AA393288 | Hs.97396 | ESTs |
| 121091 | RC_AA398894 | AA398894 | Hs.97657 | ESTs, Moderately similar to ALU8_HUMAN ALU SUBFAMILY SX SEQUENCE |
| | | | | CONTAMINATION |
| 121092 | RC_AA398895 | AA398895 | Hs.97658 | EST |
| 121094 | RC_AA398900 | AA402505 | | gb: zt62h10.r1 Soares_testis_NHT Homo sapiens cDNA clone 5′, |
| | | | | mRNA sequence |
| 121096 | RC_AA398904 | AA398904 | Hs 332690 | ESTs |
| 121115 | RC_AA399122 | AA398187 | Hs.104682 | ESTs, Weakly similar to mitochondrial citrate transport protein |
| | | | | [H sapiens] |
| 121121 | RC_AA399371 | AA399371 | Hs 189095 | similar to SALL1 (sal (Drosophila)-like |
| 121122 | RC_AA399373 | AI126713 | Hs.192233 | ESTs, Highly similar to T00337 hypothetical protein KIAA0568 |
| | | | | [H. sapiens] |
| 121125 | RC_AA399441 | AL042981 | Hs 251278 | KIAA1201 protein |
| 121151 | RC_AA399636 | AA399636 | Hs.143629 | ESTs |
| 121153 | RC_AA399640 | AA399640 | Hs.97694 | ESTs |
| 121163 | RC_AA399680 | AI676062 | Hs.111902 | ESTs |
| 121176 | RC_AA400080 | AL121523 | Hs.97774 | ESTs |
| 121192 | RC_AA400262 | AA400262 | Hs.190093 | ESTs |
| 121223 | RC_AA400725 | AI002110 | Hs.97169 | ESTs, Weakly similar to dJ667H12.2.1 [H. sapiens] |
| 121227 | RC_AA400748 | AA400748 | Hs 97823 | Homo sapiens mRNA, cDNA DKFZp434D024 (from clone DKFZp434D024) |
| 121231 | RC_AA400780 | AA814948 | Hs.96343 | ESTs, Weakly similar to ALUC_HUMAN !!!! ALU CLASS C |
| | | | | WARNING ENTRY !!! [H sapiens] |
| 121278 | RC_AA401631 | AA037121 | Hs 98518 | Homo sapiens cDNA FLJ11490 fis, clone HEMBA1001918 |
| 121279 | RC_AA401688 | AA292873 | Hs.177996 | ESTs |
| 121282 | RC_AA401695 | AA401695 | Hs.97334 | ESTs |
| 121299 | RC_AA402227 | AA402227 | Hs.22826 | tropomodulin 3 (ubiquitous) |
| 121301 | RC_AA402329 | NM_006202 | Hs.89901 | phosphodiesterase 4A, cAMP-specific (dunce (Drosophila)-homolog |
| | | | | phosphodiesterase E2) |
| 121302 | RC_AA402398 | AA402587 | Hs.325520 | LAT1-3TM protein |
| 121304 | RC_AA402449 | AA293863 | Hs.97316 | EST |
| 121305 | RC_AA402468 | AA402468 | Hs.291557 | ESTs |
| 134721 | RC_AA403268_s | AK000112 | Hs.89306 | hypothetical protein FLJ20105 |
| 121323 | RC_AA403314 | AA291411 | Hs 97247 | ESTs |
| 121324 | RC_AA404229 | AA404229 | Hs.97842 | EST |
| 129047 | RC_AA404260 | AI768623 | Hs.108264 | ESTs |
| 131074 | RC_AA404271 | U16125 | Hs.181581 | glutamate receptor, ionotropic, kainate 1 |
| 121344 | RC_AA405026 | AA405026 | Hs.193754 | ESTs |
| 121348 | RC_AA405182 | AA405182 | Hs.97973 | ESTs |
| 121350 | RC_AA405237 | AA405237 | | gb: zt06e10.s1 NCI_CGAP_GCB1 Homo sapiens cDNA clone |
| | | | | IMAGE: 712362 3′ similar to contains Alu |
| 121400 | RC_AA406061 | AA406061 | Hs.98001 | EST |
| 121402 | RC_AA406063 | AA406063 | Hs 98003 | ESTs |
| 121403 | RC_AA406070 | AA406070 | Hs 98004 | EST |
| 121408 | RC_AA406137 | AA406137 | Hs.98019 | EST |
| 121431 | RC_AA406335 | AA035279 | Hs.176731 | ESTs |
| 132936 | RC_AA411801 | AL120659 | Hs 6111 | aryl-hydrocarbon receptor nuclear translocator 2 |
| 121471 | RC_AA411804 | AA411804 | Hs.261575 | ESTs |
| 121474 | RC_AA411833 | AA402335 | Hs.188760 | ESTs, Highly similar to Trad [H. sapiens] |
| 121526 | RC_AA412219 | AW665325 | Hs 98120 | ESTs |
| 121530 | RC_AA412259 | AA778658 | Hs.98122 | ESTs |
| 121558 | RC_AA412497 | AA412497 | | gb: zt95g12.s1 Soares_testis_NHT Homo sapiens cDNA clone |
| | | | | IMAGE: 730150 3′ similar to contains L1.t3 L1 |
| 121559 | RC_AA412498 | AI192044 | Hs.104778 | ESTs |
| 121584 | RC_AA416586 | AI024471 | Hs 98232 | ESTs |
| 121609 | RC_AA416867 | AA416867 | Hs.98185 | EST |
| 121612 | RC_AA416874 | AA416874 | Hs.98168 | ESTs |
| 121737 | RC_AA421133 | AA421133 | Hs.104671 | erythrocyte transmembrane protein |
| 121740 | RC_AA421138 | AA421138 | Hs.98334 | EST |
| 129194 | RC_AA422079 | AA150797 | Hs.109276 | latexin protein |
| 121784 | RC_AA423837 | T90789 | Hs.94308 | RAB35, member RAS oncogene family |
| 121802 | RC_AA424328 | AI251870 | Hs.188898 | ESTs |
| 121803 | RC_AA424339 | AI338371 | Hs.157173 | ESTs |
| 135286 | RC_AA424469_s | AW023482 | Hs.97849 | ESTs |
| 121806 | RC_AA424502 | AA424313 | Hs.98402 | ESTs |
| 129517 | RC_AA425004 | AW972853 | Hs 112237 | ESTs |
| 121845 | RC_AA425734 | AI732692 | Hs 165066 | ESTs, Moderately similar to ALU2_HUMAN ALU SUBFAMILY SB SEQUENCE |
| | | | | CONTAMINATION |
| 121853 | RC_AA425887 | AA425887 | Hs.98502 | hypothetical protein FLJ14303 |
| 121891 | RC_AA426456 | AA426456 | Hs.98469 | ESTs |
| 121895 | RC_AA427396 | AA427396 | | gb: zw33a02.s1 Soares ovary tumor NbHOT Homo sapiens cDNA clone |
| | | | | IMAGE: 771050 3′ similar to contains |
| 121899 | RC_AA427555 | R55341 | Hs.50421 | KIAA0203 gene product |
| 121917 | RC_AA428218 | AA406397 | Hs.98038 | ESTs |
| 121918 | RC_AA428242 | BE274689 | Hs.184175 | chromosome 2 open reading frame 3 |
| 121919 | RC_AA428281 | AA428281 | Hs.98560 | EST |
| 121941 | RC_AA428865 | AA428865 | Hs.98563 | ESTs |
| 121942 | RC_AA428994 | AW452701 | Hs.293237 | ESTs |
| 121970 | RC_AA429666 | AA429666 | Hs.98617 | EST |
| 121993 | RC_AA430181 | AW297880 | Hs.98661 | ESTs |
| 134660 | RC_AA430184_s | U73524 | Hs.87465 | ATP/GTP-binding protein |
| 126753 | RC_AA431288_s | AA306478 | Hs.95327 | CD3D antigen, delta polypeptide (TiT3 complex) |
| 122022 | RC_AA431293 | AA431293 | Hs.98716 | ESTs, Moderately similar to T42650 hypothetical protein |
| | | | | DKFZp434D0215.1 [H sapiens] |
| 122050 | RC_AA431478 | AI453076 | Hs.166109 | ELAV (embryonic lethal, abnormal vision, Drosophila)-like 2 |
| 122051 | RC_AA431492 | AA431492 | Hs 98742 | EST |
| 122055 | RC_AA431732 | AA431732 | Hs.98747 | EST |
| 122105 | RC_AA432278 | AW241685 | Hs.98699 | ESTs |
| 122125 | RC_AA434411 | AK000492 | Hs 98806 | hypothetical protein |
| 135235 | RC_AA435512_i | AW298244 | Hs 293507 | ESTs |
| 122162 | RC_AA435698 | AA628233 | Hs.79946 | cytochrome P450, subfamily XIX (aromatization of androgens) |
| 129406 | RC_AA435711 | AB018255 | Hs.111138 | KIAA0712 gene product |
| 318801 | RC_AA435815_s | U40763 | Hs.77965 | peptidyl-prolyl isomerase G (cyclophilin G) |
| 122186 | RC_AA435842 | AA398811 | Hs 104673 | ESTs |
| 122235 | RC_AA436475 | AA436475 | Hs.112227 | membrane-associated nucleic acid binding protein |
| 129131 | RC_AA436489 | AB026436 | Hs.177534 | dual specificity phosphatase 10 |
| 134664 | RC_AA442060 | AA256106 | Hs.87507 | ESTs |
| 122310 | RC_AA442079 | AW192803 | Hs.98974 | ESTs, Weakly similar to S65824 reverse transcriptase homolog |
| | | | | [H. sapiens] |
| 122334 | RC_AA443151 | BE465894 | Hs 98365 | ESTs, Weakly similar to LB4D_HUMAN NADP-DEPENDENT LEUKOTRIENE B4 |
| | | | | 12- |
| 122382 | RC_AA446133 | AA446440 | Hs 98643 | ESTs |
| 122425 | RC_AA447145 | AB007859 | Hs.100955 | KIAA0399 protein |
| 122431 | RC_AA447398 | AA447398 | Hs 99104 | ESTs |
| 122450 | RC_AA447643 | AA447643 | Hs.112095 | hypothetical protein DKFZp434F1819 |
| 302653 | RC_AA447742_s | AJ404468 | Hs.284259 | dynein, axonemal, heavy polypeptide 9 |
| 122477 | RC_AA448226 | AA448226 | Hs.324123 | ESTs |
| 122500 | RC_AA448825 | AA448825 | Hs.99190 | ESTs |
| 122522 | RC_AA449444 | AA299607 | Hs.98969 | ESTs |
| 122536 | RC_AA450087 | AF060877 | Hs.99236 | regulator of G-protein signalling 20 |
| 122538 | RC_AA450211 | AA450211 | Hs.99239 | ESTs |
| 122540 | RC_AA450244 | AA476741 | Hs.98279 | ESTs, Weakly similar to A43932 mucin 2 precursor, intestinal |
| | | | | [H sapiens] |
| 122560 | RC_AA452123 | AW392342 | Hs.283077 | centrosomal P4.1-associated protein; uncharacterized bone marrow |
| | | | | protein BM032 |
| 421919 | RC_AA452155 | AJ224901 | Hs.109526 | zinc finger protein 198 |
| 122562 | RC_AA452156 | AA452156 | | gb: zx29c03.s1 Soares_total_fetus_Nb2HF8_9w Homo sapiens cDNA clone |
| | | | | IMAGE: 787876 3′, mRNA |
| 122585 | RC_AA453036 | AI681654 | Hs.170737 | hypothetical protein FLJ23251 |
| 122608 | RC_AA453526 | AA453525 | Hs.143077 | ESTs |
| 122635 | RC_AA454085 | AA454085 | | gb: zx33a08 s1 Soares_total_fetus_Nb2HF8_9w Homo sapiens cDNA clone |
| | | | | IMAGE: 788246 3′ similar to |
| 122636 | RC_AA454103 | AW651706 | Hs.99519 | hypothetical protein FLJ14007 |
| 122653 | RC_AA454642 | AW009166 | Hs.99376 | ESTs |
| 122660 | RC_AA454935 | AI816827 | Hs.180069 | nuclear respiratory factor 1 |
| 122703 | RC_AA456323 | AA456323 | Hs 269369 | ESTs |
| 122724 | RC_AA457395 | AA457395 | Hs.99457 | ESTs |
| 122749 | RC_AA458850 | AA458850 | Hs.293372 | ESTs, Weakly similar to B34087 hypothetical protein |
| | | | | [H. sapiens] |
| 122772 | RC_AA459662 | AW117452 | Hs.99489 | ESTs |
| 131098 | RC_AA459668 | U66669 | Hs.236642 | 3-hydroxyisobutyryl-Coenzyme A hydrolase |
| 129045 | RC_AA459679_s | AI082883 | Hs.30732 | hypothetical protein FLJ13409; KIAA1711 protein |
| 122777 | RC_AA459702 | AK001022 | Hs.214397 | hypothetical protein FLJ10160 similar to insulin related |
| | | | | protein 2 |
| 135362 | RC_AA460017_f | AA978128 | Hs.99513 | ESTs, Weakly similar to T17454 diaphanous-related formin - mouse |
| | | | | [M. musculus] |
| 122798 | RC_AA460324 | AW366286 | Hs 145696 | splicing factor (CC1.3) |
| 122837 | RC_AA461509 | AA461509 | Hs.293565 | ESTs, Weakly similar to putative p150 [H. sapiens] |
| 122860 | RC_AA464414_i | AA464414 | | gb: zx78g01.s1 Soares ovary tumor NbHOT Homo sapiens cDNA clone |
| | | | | IMAGE: 809904 3′, mRNA sequence. |
| 122861 | RC_AA464428 | AA335721 | Hs.119394 | ESTs |
| 122910 | RC_AA470084 | AA470084 | Hs.98358 | ESTs |
| 132899 | RC_AA476606_s | AA476606 | Hs 59666 | SMAD in the antisense orientation |
| 122967 | RC_AA478521 | AA806187 | Hs 289101 | glucose regulated protein, 58 kD |
| 129560 | RC_AA478523 | AA317841 | Hs.7845 | hypothetical protein MGC2752 |
| 123009 | RC_AA479949 | AA535244 | Hs.78305 | RAB2, member RAS oncogene family |
| 128917 | RC_AA481252 | AI365215 | Hs 206097 | oncogene TC21 |
| 123081 | RC_AA485351 | AI815486 | Hs.243901 | Homo sapiens cDNA FLJ20738 fis, clone HEP08257 |
| 123133 | RC_AA487264 | AA487264 | Hs.154974 | Homo sapiens mRNA; cDNA DKFZp667N064 (from clone DKFZp667N064) |
| 123184 | RC_AA489072 | BE247767 | Hs.18166 | KIAA0870 protein |
| 129671 | RC_AA489630 | NM_014700 | Hs.119004 | KIAA0665 gene product |
| 123233 | RC_AA490225 | AW974175 | Hs.188751 | ESTs, Weakly similar to MAPB_HUMAN MICROTUBULE-ASSOCIATED PROTEIN |
| | | | | 1B [H. sapiens] |
| 123234 | RC_AA490227 | NM_001938 | Hs.16697 | down-regulator of transcription 1, TBP-binding (negative |
| | | | | cofactor 2) |
| 123236 | RC_AA490255 | AW968504 | Hs.123073 | CDC2-related protein kinase 7 |
| 123255 | RC_AA490890 | AA830335 | Hs.105273 | ESTs |
| 129503 | RC_AA490916_s | AW768399 | Hs.112157 | ESTs |
| 131043 | RC_AA490925 | AF084535 | Hs.22464 | epilepsy, progressive myoclonus type 2, Lafora disease (laforin) |
| 123259 | RC_AA490955 | AI744152 | Hs 283374 | ESTs, Weakly similar to CA15_HUMAN COLLAGEN ALPHA 1(V) CHAIN |
| | | | | PRECURSOR [H. sapiens] |
| 123284 | RC_AA495812 | AA488988 | Hs 293796 | ESTs |
| 123286 | RC_AA495824 | AA495824 | Hs.188822 | ESTs, Weakly similar to A46010 X-linked retinopathy protein |
| | | | | [H sapiens] |
| 123315 | RC_AA496369 | AA496369 | | gb: zv37d10.s1 Soares ovary tumor NbHOT Homo sapiens cDNA clone |
| | | | | IMAGE: 755827 3′ similar to contains |
| 129179 | RC_AA504125_s | AW969025 | Hs 109154 | ESTs |
| 131612 | RC_AA521473 | AU076668 | Hs 334884 | SEC10 (S. cerevisiae)-like 1 |
| 123421 | RC_AA598440 | AA598440 | Hs.291154 | EST, Weakly similar to I38022 hypothetical protein |
| | | | | [H. sapiens] |
| 123449 | RC_AA598899_i | AL049325 | Hs.112493 | Homo sapiens mRNA; cDNA DKFZp564D036 (from clone DKFZp564D036) |
| 129021 | RC_AA599244 | AL044675 | Hs.173081 | KIAA0530 protein |
| 132830 | RC_AA599694_s | NM_014777 | Hs.57730 | KIAA0133 gene product |
| 123497 | RC_AA600037 | AA765256 | Hs 135191 | ESTs, Weakly similar to unnamed protein product [H. sapiens] |
| 123604 | RC_AA609135 | AA609135 | Hs.293076 | ESTs |
| 129539 | RC_AA609582 | T47614 | Hs.323022 | ESTs, Highly similar to p60 katanin [H. sapiens] |
| 123712 | RC_AA609684 | AA609684 | Hs.112748 | Homo sapiens cDNA: FLJ21543 fis, clone COL06171 |
| 123731 | RC_AA609839 | AA609839 | | gb: ae62f01.s1 Stratagene lung carcinoma 937218 Homo sapiens cDNA |
| | | | | clone IMAGE: 951481 3′ similar to |
| 130725 | RC_AA609862 | T98807 | Hs.80248 | RNA-binding protein gene with multiple splicing |
| 123800 | RC_AA620423 | AA620423 | Hs.112862 | EST |
| 123841 | RC_AA620747 | AA620747 | Hs.112896 | ESTs |
| 123929 | RC_AA621364 | AA621364 | Hs.112981 | ESTs |
| 123978 | RC_C20653 | T89832 | Hs.170278 | ESTs |
| 133184 | RC_D20085 | AA001021 | Hs.6685 | thyroid hormone receptor interactor 8 |
| 132835 | RC_D20749 | Z83844 | Hs.5790 | hypothetical protein dJ37E16.5 |
| 132406 | RC_D51285_s | AL133731 | Hs.4774 | Homo sapiens mRNA, cDNA DKFZp761C1712 (from clone DKFZp761C1712) |
| 128695 | RC_D59972_i | NM_003478 | Hs.101299 | cullin 5 |
| 124028 | RC_F04112_f | F04112 | | gb: HSC2JH062 normalized infant brain cDNA Homo sapiens cDNA clone |
| | | | | c-2jh06 3′, mRNA sequence. |
| 124057 | RC_F13604 | AA902384 | Hs 73853 | bone morphogenetic protein 2 |
| 134899 | RC_H01662 | AI609045 | Hs.321775 | hypothetical protein DKFZp434D1428 |
| 130973 | RC_H05135_i | AI638418 | Hs.78580 | DEAD/H (Asp-Glu-Ala-Asp/His) box polypeptide 1 |
| 124106 | RC_H12245 | H12245 | | gb: ym17a12.r1 Soares infant brain 1NIB Homo sapiens cDNA clone |
| | | | | 3′, mRNA sequence |
| 124136 | RC_H22842 | H22842 | Hs.101770 | EST |
| 124165 | RC_H30894 | H30039 | Hs.107674 | ESTs |
| 131229 | RC_H43442_s | NM_015340 | Hs 2450 | leucyl-tRNA synthetase, mitochondrial |
| 124178 | RC_H45996 | BE463721 | Hs.97101 | putative G protein-coupled receptor |
| 129948 | RC_H69281_i | AI537162 | Hs.263988 | ESTs |
| 134374 | RC_H69485_f | N22687 | Hs.8236 | ESTs |
| 124254 | RC_H69899 | H69899 | | gb: yu70c12 s1 Weizmann Olfactory Epithelium Homo sapiens cDNA |
| | | | | clone IMAGE: 239158 3′ similar to |
| 129056 | RC_H70627_s | AI769958 | Hs.108336 | ESTs, Weakly similar to ALUE_HUMAN !!!! ALU CLASS E |
| | | | | WARNING ENTRY !!! [H. sapiens] |
| 100919 | RC_H73050_s | X54534 | Hs 278994 | Rhesus blood group, CcEe antigens |
| 130724 | RC_H73260 | AK001507 | Hs.306084 | Homo sapiens clone FLB6914 PRO1821 mRNA, complete cds |
| 100716 | RC_H77531_s | X89887 | Hs.172350 | HIR (histone cell cycle regulation defective, S. cerevisiae) |
| | | | | homolog A |
| 124274 | RC_H80552 | H80552 | Hs 102249 | EST |
| 129078 | RC_H80737_s | AI351010 | Hs.102267 | lysosomal |
| 124828 | RC_H93412 | AW952124 | Hs.13094 | presenilins associated rhomboid-like protein |
| 124315 | RC_H94892_s | NM_005402 | Hs.288757 | v-ral simian leukemia viral oncogene homolog A (ras related) |
| 100747 | RC_H95643_s | X04588 | Hs.85844 | neurotrophic tyrosine kinase, receptor, type 1 |
| 124324 | RC_H96552 | H96552 | Hs.159472 | Homo sapiens cDNA. FLJ22224 fis, clone HRC01703 |
| 452933 | RC_H97146 | AW391423 | Hs.288555 | Homo sapiens cDNA: FLJ22425 fis, clone HRC08686 |
| 132231 | RC_H99131_s | AA662910 | Hs.42635 | hypothetical protein DKFZp434K2435 |
| 129170 | RC_H99462_s | AW250380 | Hs.109059 | mitochondrial ribosomal protein L12 |
| 133143 | RC_H99837_s | AA094538 | Hs.272808 | putative transcription regulation nuclear protein; KIAA1689 |
| | | | | protein |
| 132963 | RC_N22140 | AA099693 | Hs.34851 | epsilon-tubulin |
| 135297 | RC_N22197 | AL118782 | Hs.300208 | Sec23-interacting protein p125 |
| 134347 | RC_N23756_s | AF164142 | Hs 82042 | solute carrier family 23 (nucleobase transporters), member 1 |
| 130365 | RC_N24134 | W56119 | Hs.155103 | eukaryotic translation initiation factor 1A, Y chromosome |
| 421642 | RC_N24195 | AF172066 | Hs.106346 | retinoic acid repressive protein |
| 439311 | RC_N26739 | BE270668 | Hs.151945 | mitochondrial ribosomal protein L43 |
| 124383 | RC_N27098 | N27098 | Hs.102463 | EST |
| 124387 | RC_N27637 | N27637 | Hs.109019 | ESTs |
| 129341 | RC_N33090 | AI193519 | Hs.226396 | hypothetical protein FLJ11126 |
| 129081 | RC_N35967 | AI364933 | Hs.168913 | serine/threonine kinase 24 (Ste20, yeast homolog) |
| 102827 | RC_N38959_f | BE244588 | Hs 6456 | chaperonin containing TCP1, subunit 2 (beta) |
| 124433 | RC_N39069 | AA280319 | Hs 288840 | PRO1575 protein |
| 124441 | RC_N46441 | AW450481 | Hs.161333 | ESTs |
| 132338 | RC_N48270_f | AA353868 | Hs.182982 | golgin-67 |
| 131403 | RC_N48365_s | AI473114 | Hs 26455 | ESTs |
| 124466 | RC_N51316 | R10084 | Hs.113319 | kinesin heavy chain member 2 |
| 132210 | RC_N51499_s | NM_007203 | Hs.42322 | A kinase (PRKA) anchor protein 2 |
| 124483 | RC_N53976 | AI821780 | Hs.179864 | ESTs |
| 124484 | RC_N54157 | H66118 | Hs.285520 | ESTs, Weakly similar to 2109260A B cell growth factor |
| | | | | [H. sapiens] |
| 124485 | RC_N54300 | AB040933 | Hs.15420 | KIAA1500 protein |
| 124494 | RC_N54831 | N54831 | Hs 271381 | ESTs, Weakly similar to I38022 hypothetical protein |
| | | | | [H. sapiens] |
| 129200 | RC_N59849 | N59849 | Hs.13565 | Sam68-like phosphotyrosine protein, T-STAR |
| 124527 | RC_N62132 | N79264 | Hs.269104 | ESTs |
| 124532 | RC_N62375 | N62375 | Hs.102731 | EST |
| 133213 | RC_N63138 | AA903424 | Hs.6786 | ESTs |
| 124539 | RC_N63172 | D54120 | Hs 146409 | cell division cycle 42 (GTP-binding protein, 25 kD) |
| 133651 | RC_N63772 | AI301740 | Hs.173381 | dihydropyrimidinase-like 2 |
| 129196 | RC_N63787 | BE296313 | Hs 265592 | ESTs, Weakly similar to I38022 hypothetical protein |
| | | | | [H. sapiens] |
| 124575 | RC_N68168 | N68168 | | gb: za11c01.s1 Soares fetal liver spleen 1NFLS Homo sapiens cDNA |
| | | | | clone 3′, mRNA sequence |
| 124576 | RC_N68201 | N68201 | Hs 269124 | ESTs, Weakly similar to I38022 hypothetical protein |
| | | | | [H sapiens] |
| 124577 | RC_N68300 | N68300 | | gb: za12g07.s1 Soares fetal liver spleen 1NFLS Homo sapiens cDNA |
| | | | | clone IMAGE: 292380 3′, mRNA |
| 124578 | RC_N68321 | N68321 | Hs.231500 | EST |
| 124593 | RC_N69575 | N69575 | Hs.102788 | ESTs |
| 128501 | RC_N75007 | AL133572 | Hs.199009 | protein containing CXXC domain 2 |
| 105691 | RC_N75542 | AI680737 | Hs.289068 | Homo sapiens cDNA FLJ11918 fis, clone HEMBB1000272 |
| 128473 | RC_N90066 | T78277 | Hs.100293 | O-linked N-acetylglucosamine (GlcNAc) transferase (UDP-N- |
| | | | | acetylglucosamine: polypeptide-N- |
| 128639 | RC_N91246 | AW582962 | Hs.102897 | CGI-47 protein |
| 124652 | RC_N92751 | W19407 | Hs.3862 | regulator of nonsense transcripts 2; DKFZP434D222 protein |
| 133137 | RC_N93214_s | AB002316 | Hs.65746 | KIAA0318 protein |
| 124671 | RC_N99148 | AK001357 | Hs.102951 | Homo sapiens cDNA FLJ10495 fis, clone NT2RP2000297, moderately |
| | | | | similar to ZINC FINGER PROTEIN |
| 133054 | RC_R07876 | AA464836 | Hs.291079 | ESTs, Weakly similar to T27173 hypothetical protein Y54G11A.9 - |
| | | | | Caenorhabditis elegans [C. elegans] |
| 130410 | RC_R10865_f | J00077 | Hs 155421 | alpha-fetoprotein |
| 124720 | RC_R11056 | R05283 | | gb: ye91c08.s1 Soares fetal liver spleen 1NFLS Homo sapiens cDNA |
| | | | | clone IMAGE: 125102 3′ similar to |
| 124722 | RC_R11488 | T97733 | Hs.185685 | ESTs |
| 129961 | RC_R22947 | R23053 | | gb: yh31a05.r1 Soares placenta Nb2HP Homo sapiens cDNA clone 5′ |
| | | | | similar to contains L1 repetitive element |
| 128944 | RC_R23930_s | AL137586 | Hs 52763 | anaphase-promoting complex subunit 7 |
| 132965 | RC_R26589_f | AI248173 | Hs.191460 | hypothetical protein MGC12936 |
| 133740 | RC_R37588_s | AW162919 | Hs 170160 | RAB2, member RAS oncogene family-like |
| 133074 | RC_R37613 | AL134275 | Hs 6434 | hypothetical protein DKFZp761F2014 |
| 124757 | RC_R38398 | H11368 | Hs.141055 | Homo sapiens clone 23758 mRNA sequence |
| 124762 | RC_R39179_f | AA553722 | Hs.92096 | ESTs, Moderately similar to A46010 X-linked retinopathy protein |
| | | | | [H sapiens] |
| 124773 | RC_R40923 | R45154 | Hs 106604 | ESTs |
| 135266 | RC_R41179 | R41179 | Hs 97393 | KIAA0328 protein |
| 131375 | RC_R41294_s | AW293165 | Hs 143134 | ESTs |
| 133753 | RC_R42307_f | NM_004427 | Hs.165263 | early development regulator 2 (homolog of polyhomeotic 2) |
| 128540 | RC_R43189_f | AW297929 | Hs.328317 | EST |
| 124785 | RC_R43306 | W38537 | Hs.280740 | hypothetical protein MGC3040 |
| 124792 | RC_R44357 | R44357 | Hs.48712 | hypothetical protein FLJ20736 |
| 124793 | RC_R44519 | R44519 | | gb: yg24h04.s1 Soares infant brain 1NIB Homo sapiens cDNA clone |
| | | | | IMAGE: 33350 3′, mRNA sequence. |
| 124799 | RC_R45088 | R45088 | | gb: yg38g04.s1 Soares infant brain 1NIB Homo sapiens cDNA clone |
| | | | | IMAGE 34896 3′, mRNA sequence. |
| 124812 | RC_R47948_i | R47948 | Hs 188732 | ESTs |
| 124821 | RC_R51524 | H87832 | Hs.7388 | kelch (Drosophila)-like 3 |
| 127274 | RC_R54950 | AW966158 | Hs.58582 | Homo sapiens cDNA FLJ12789 fis, clone NT2RP2001947 |
| 124835 | RC_R55241 | R55241 | Hs.101214 | EST |
| 124845 | RC_R59585 | R59585 | Hs 101255 | ESTs |
| 124847 | RC_R60044 | W07701 | Hs.304177 | Homo sapiens clone FLB8503 PRO2286 mRNA, complete cds |
| 440630 | RC_R60872 | BE561430 | Hs 239388 | Human DNA sequence from clone RP1-304B14 on chromosome 6. Contains |
| | | | | a gene for a novel protein and a part of a gene for a novel |
| | | | | protein with two isoforms. Contains ESTs, STSs, GSSs and a CpG |
| | | | | island |
| 124861 | RC_R66690 | R67567 | Hs.107110 | ESTs |
| 130141 | RC_R67266_s | NM_004455 | Hs.150956 | exostoses (multiple)-like 1 |
| 124879 | RC_R73588 | R73588 | Hs.101533 | ESTs |
| 124892 | RC_R79403 | AI970003 | Hs.23756 | hypothetical protein similar to swine acylneuraminate lyase |
| 124906 | RC_R87647 | H75964 | Hs.107815 | ESTs |
| 124922 | RC_R93622 | R93622 | Hs.12163 | eukaryotic translation initiation factor 2, subunit 2 (beta, 38 |
| | | | | kD) |
| 124940 | RC_R99599_s | AF068846 | Hs.103804 | heterogeneous nuclear ribonucleoprotein U (scaffold attachment |
| | | | | factor A) |
| 124941 | RC_R99612 | AI766661 | Hs.27774 | ESTs, Highly similar to AF161349 1 HSPC086 [H. sapiens] |
| 124943 | RC_T02888 | AW963279 | Hs.123373 | ESTs, Weakly similar to ALU1_HUMAN ALU SUBFAMILY J SEQUENCE |
| | | | | CONTAMINATION WARNING ENTRY [H sapiens] |
| 124947 | RC_TJ3170 | T03170 | Hs.100165 | ESTs |
| 124954 | RC_T10465 | AW964237 | Hs.6728 | KIAA1548 protein |
| 132924 | RC_T15418_f | U55184 | Hs.154145 | hypothetical protein FLJ11585 |
| 133113 | RC_T15597_f | BE383768 | Hs 65238 | 95 kDa retinoblastoma protein binding protein, KIAA0661 gene |
| | | | | product |
| 132975 | RC_T15652_i | R43504 | Hs.6181 | ESTs |
| 133235 | RC_T16898_s | AW960782 | Hs 6856 | ash2 (absent, small, or homeotic, Drosophila, homolog)-like |
| 131082 | RC_T26644_i | AI091121 | Hs.246218 | Homo sapiens cDNA: FLJ21781 fis, clone HEP00223 |
| 124980 | RC_T40841 | T40841 | Hs.98681 | ESTs |
| 124984 | RC_T47566_i | BE313210 | Hs.223241 | eukaryotic translation elongation factor 1 delta (guanine |
| | | | | nucleotide exchange protein) |
| 124991 | RC_T50116 | T50116 | | gb: yb77c10.s1 Stratagene ovary (937217) Homo sapiens cDNA clone |
| | | | | IMAGE: 77202 3′ similar to similar to SP: VE22_LAMBD P03756 EA22 |
| | | | | GENE, mRNA sequence. |
| 129475 | RC_T50145_s | NM_004477 | Hs.203772 | FSHD region gene 1 |
| 125000 | RC_T58615 | T58615 | Hs.110640 | ESTs |
| 132932 | RC_T59940_f | AW118826 | Hs.6093 | Homo sapiens cDNA: FLJ22783 fis, clone KAIA1993 |
| 129534 | RC_T63595 | AK002126 | Hs.11260 | hypothetical protein FLJ11264 |
| 125008 | RC_T64891 | T91251 | | gb: yd60a10.s1 Soares fetal liver spleen 1NFLS Homo sapiens cDNA |
| | | | | clone 3′, mRNA sequence |
| 125009 | RC_T64924 | T64924 | Hs.303046 | ESTs |
| 132940 | RC_T64933_r | T79136 | Hs.127243 | Homo sapiens mRNA for KIAA1724 protein, partial cds |
| 125017 | RC_T68875 | T68875 | | gb: yc30f05.s1 Stratagene liver (937224) Homo sapiens cDNA clone |
| | | | | IMAGE 82209 3′, mRNA sequence. |
| 125018 | RC_T69027 | T69027 | Hs 57475 | sex comb on midleg homolog 1 |
| 125020 | RC_T69924 | T69981 | | gb: yc19d03.r1 Stratagene lung (937210) Homo sapiens cDNA clone |
| | | | | 5′, mRNA sequence |
| 129891 | RC_T70353 | AI084813 | Hs.13197 | ESTs |
| 134204 | RC_T79780_s | AI873257 | Hs.7994 | hypothetical protein FLJ20551 |
| 125050 | RC_T79951 | AW970209 | Hs.111805 | ESTs |
| 125052 | RC_T80174_s | T85104 | Hs.222779 | ESTs, Moderately similar to similar to NEDD-4 [H. sapiens] |
| 125054 | RC_T80622 | T80622 | Hs.268601 | ESTs, Weakly similar to envelope [H. sapiens] |
| 125063 | RC_T85352 | T85352 | | gb: yd82d01.s1 Soares fetal liver spleen 1NFLS Homo sapiens cDNA |
| | | | | clone IMAGE 114721 3′ similar to contains Alu repetitive |
| | | | | element; contains L1 repetitive element;, mRNA sequence. |
| 125064 | RC_T85373 | T85373 | | gb: yd82f07.s1 Soares fetal liver spleen 1NFLS Homo sapiens cDNA |
| | | | | clone IMAGE: 114757 3′ similar to contains Alu repetitive |
| | | | | element, contains MER3 repetitive element;, mRNA sequence. |
| 125066 | RC_T86284 | T86284 | | gb: yd77b07.s1 Soares fetal liver spleen 1NFLS Homo sapiens cDNA |
| | | | | clone 3′ similar to contains Alu repetitive element;, mRNA |
| | | | | sequence |
| 112264 | RC_T89579_s | AL045364 | Hs 79353 | transcription factor Dp-1 |
| 125080 | RC_T90360 | T90360 | Hs.268620 | ESTs, Highly similar to ALU6_HUMAN ALU SUBFAMILY SP SEQUENCE |
| | | | | CONTAMINATION WARNING ENTRY [H. sapiens] |
| 125097 | RC_T94328_i | AW576389 | Hs 335774 | EST, Moderately similar to S65657 alpha-1C-adrenergic receptor |
| | | | | splice form 2 [H sapiens] |
| 125104 | RC_T95590 | T95590 | | gb: ye40a03 s1 Soares fetal liver spleen 1NFLS Homo sapiens cDNA |
| | | | | clone 3′ similar to gb\|M10817\|IGURRAA Iguana iguana |
| | | | | 5S (rRNA);, mRNA sequence |
| 135107 | RC_T97257_f | T97257 | Hs.337531 | ESTs, Moderately similar to I38022 hypothetical protein |
| | | | | [H. sapiens] |
| 129550 | RC_T97599_i | AA845462 | Hs.124024 | deltex (Drosophila) homolog 1 |
| 125118 | RC_T97620 | R10606 | | gb: yf35f11.s1 Soares fetal liver spleen 1NFLS Homo sapiens cDNA |
| | | | | clone IMAGE: 128877 3′ similar to contains Alu repetitive |
| | | | | element,, mRNA sequence. |
| 125120 | RC_T97775 | T97775 | Hs.100717 | EST |
| 134160 | RC_T98152 | T98152 | Hs.79432 | fibrillin 2 (congenital contractural arachnodactyly) |
| 125136 | RC_W31479 | AW962364 | Hs.129051 | ESTs |
| 125144 | RC_W37999 | AB037742 | Hs.24336 | KIAA1321 protein |
| 125150 | RC_W38240 | W38240 | | Empirically selected from AFFX single probeset |
| 104180 | RC_W40150 | AA247778 | Hs.119155 | Homo sapiens mRNA full length insert cDNA clone EUROIMAGE 814975 |
| 131987 | RC_W45435 | AW453069 | Hs 3657 | activity-dependent neuroprotective protein |
| 125178 | RC_W58202 | W93127 | Hs.31845 | ESTs |
| 125180 | RC_W58344 | W58469 | Hs.103120 | ESTs |
| 125182 | RC_W58650 | AA451755 | Hs.263560 | ESTs |
| 130588 | RC_W68736 | AL030996 | Hs.16411 | hypothetical protein LOC57187 |
| 125197 | RC_W69106 | AF086270 | Hs.278554 | heterochromatin-like protein 1 |
| 133497 | RC_W69111 | BE617303 | Hs.74266 | hypothetical protein MGC4251 |
| 100562 | RC_W69385_s | NM_006185 | Hs 301512 | nuclear mitotic apparatus protein 1 |
| 125639 | RC_W69399_s | Z97630 | Hs.226117 | H1 histone family, member 0 |
| 129232 | RC_W69459 | R98881 | Hs.109655 | sex comb on midleg (Drosophila)-like 1 |
| 101495 | RC_W72424 | W72424 | Hs.112405 | S100 calcium-binding protein A9 (calgranulin B) |
| 125209 | RC_W72724 | W72724 | Hs.103174 | ESTs, Weakly similar to TSP2_HUMAN THROMBOSPONDIN 2 PRECURSOR |
| | | | | [H. sapiens] |
| 125212 | RC_W72834 | AA746225 | Hs.103173 | ESTs |
| 129132 | RC_W73955 | BE383436 | Hs.108847 | hypothetical protein MGC2749 |
| 125223 | RC_W74701 | AI916269 | Hs.109057 | ESTs, Weakly similar to ALU5_HUMAN ALU SUBFAMILY SC SEQUENCE |
| | | | | CONTAMINATION WARNING ENTRY [H. sapiens] |
| 125225 | RC_W76540 | W74169 | Hs 16492 | DKFZP564G2022 protein |
| 125228 | RC_W79397 | AA033982 | Hs.110059 | ESTs, Weakly similar to I38022 hypothetical protein |
| | | | | [H. sapiens] |
| 132393 | RC_W85888 | AL135094 | Hs.47334 | hypothetical protein FLJ14495 |
| 125238 | RC_W86038 | N99713 | Hs.109514 | ESTs |
| 125247 | RC_W86881 | AA694191 | Hs.163914 | ESTs |
| 129296 | RC_W87804 | AI051967 | Hs.110122 | ESTs |
| 125263 | RC_W88942 | AA098878 | | gb: zn45g10 r1 Stratagene HeLa cell s3 937216 Homo sapiens cDNA |
| | | | | clone 5′, mRNA sequence |
| 125266 | RC_W90022 | W90022 | Hs.186809 | ESTs, Highly similar to LCT2_HUMAN LEUKOCYTE CELL-DERIVED |
| | | | | CHEMOTAXIN 2 PRECURSOR [H. sapiens] |
| 131321 | RC_W92272 | U91543 | Hs.25601 | chromodomain helicase DNA binding protein 3 |
| 131601 | RC_W92764_s | NM_007115 | Hs.29352 | tumor necrosis factor, alpha-induced protein 6 |
| 131677 | RC_W93040 | H05317 | Hs 283549 | ESTs |
| 120837 | RC_W93092 | BE149656 | Hs.306621 | Homo sapiens cDNA FLJ11963 fis, clone HEMBB1001051 |
| 125277 | RC_W93227 | W93227 | Hs.103245 | EST |
| 125278 | RC_W93523 | AI218439 | Hs 129998 | enhancer of polycomb 1 |
| 125280 | RC_W93659 | AI123705 | Hs.106932 | ESTs |
| 131856 | RC_W94003_s | W93949 | Hs 33245 | ESTs |
| 131844 | RC_W94401_s | AI419294 | Hs.324342 | ESTs |
| 125284 | RC_W94688 | NM_002666 | Hs.103253 | penlipin |
| 313447 | RC_W94787_s | AW016321 | Hs.82306 | destrin (actin depolymerizing factor) |
| 130799 | RC_Z38294_s | AB028945 | Hs.12696 | cortactin SH3 domain-binding protein |
| 125289 | RC_Z38311 | T34530 | Hs.4210 | Homo sapiens cDNA FLJ13069 fis, clone NT2RP3001752 |
| 128874 | RC_Z38465_s | H06245 | Hs.106801 | ESTs, Weakly similar to PC4259 ferritin associated protein |
| | | | | [H. sapiens] |
| 130966 | RC_Z38525_s | AW971018 | Hs.21659 | ESTs |
| 128875 | RC_Z38538_f | AB040923 | Hs.106808 | kelch (Drosophila)-like 1 |
| 133200 | RC_Z38551_s | AB037715 | Hs.183639 | hypothetical protein FLJ10210 |
| 130158 | RC_Z38783_s | AB032947 | Hs 151301 | Ca2 +-dependent activator protein for secretion |
| 125295 | RC_Z39113 | AB022317 | Hs 25887 | sema domain, immunoglobulin domain (Ig), transmembrane domain (TM) |
| | | | | and short cytoplasmic domain, (semaphorin) 4F |
| 125298 | RC_Z39255_f | AW972542 | Hs.289008 | Homo sapiens cDNA: FLJ21814 fis, clone HEP01068 |
| 125300 | RC_Z39591 | Z39591 | Hs.101376 | EST |
| 323122 | RC_Z39783_s | BE622770 | Hs.264915 | Homo sapiens cDNA FLJ12908 fis, clone NT2RP2004399 |
| 311463 | RC_Z39920 | R55344 | Hs.22142 | cytochrome b5 reductase b5R.2 |
| 130882 | RC_Z40166_f | AA497044 | Hs 20887 | hypothetical protein FLJ10392 |
| 128888 | RC_Z40388_s | AI760853 | Hs.241558 | ariadne (Drosophila) homolog 2 |
| 125310 | RC_Z40646 | R59161 | Hs 124953 | ESTs |
| 125315 | RC_Z41697 | R38110 | Hs.106296 | ESTs |
| 125317 | RC_Z99349 | Z99348 | Hs.112461 | ESTs, Weakly similar to I38022 hypothetical protein |
| | | | | [H. sapiens] |
| 135096 | RC_Z99394_s | AA081258 | Hs.132390 | zinc finger protein 36 (KOX 18) |
| 104786 | RC_AA027168 | AA027167 | Hs.10031 | KIAA0955 protein |
| 132837 | D58024_s | AA370362 | Hs 57958 | EGF-TM7-latrophilin-related protein |
| 120456 | RC_AA251113 | AA488750 | Hs.88414 | BTB and CNC homology 1, basic leucine zipper transcription |
| | | | | factor 2 |
| 132459 | RC_AA347573 | AL120071 | Hs.48998 | fibronectin leucine rich transmembrane protein 2 |
| 101545 | M31210 | BE246154 | Hs.154210 | endothelial differentiation, sphingolipid G-protein-coupled |
| | | | | receptor, 1 |
| 133505 | C01527 | AI630124 | Hs 324504 | Homo sapiens mRNA; cDNA DKFZp586J0720 (from clone DKFZp586J0720) |
| 132360 | RC_N62948_s | AW893660 | Hs.46440 | solute carrier family 21 (organic anion transporter), member 3 |
| 132738 | RC_W42674 | AK000738 | Hs 264636 | hypothetical protein FLJ20731 |
| 119586 | RC_W43000_s | AF088033 | Hs.159225 | ESTs |
| 129914 | RC_N31750_s | NM_012421 | Hs.13321 | rearranged L-myc fusion sequence |
| 130839 | AF009301 | AB011169 | Hs.20141 | similar to S cerevisiae SSM4 |
| 132813 | L37347 | BE313625 | Hs.57435 | solute carrier family 11 (proton-coupled divalent metal ion |
| | | | | transporters), member 2 |
| 134342 | M99564 | NM_000275 | Hs.82027 | oculocutaneous albinism II (pink-eye dilution (murine) homolog) |
| 131878 | RC_AA430673 | AA083764 | Hs.6101 | hypothetical protein MGC3178 |
| 105426 | RC_AA251297 | W20027 | Hs 23439 | ESTs |
| 132968 | RC_AA620722 | AF234532 | Hs 61638 | myosin X |
| 132173 | RC_W46577_s | X89426 | Hs 41716 | endothelial cell-specific molecule 1 |
| 113932 | RC_W81237 | AA256444 | Hs 126485 | hypothetical protein FLJ12604; KIAA1692 protein |
| 114452 | RC_AA020825 | AI369275 | Hs.243010 | Homo sapiens cDNA FLJ14445 fis, clone HEMBB1001294, highly |
| | | | | similar to GTP-BINDING PROTEIN TC10 |
| 115243 | RC_AA278766 | AA806600 | Hs.116665 | KIAA1842 protein |
| 134403 | RC_H93708_s | AA334551 | Hs 82767 | sperm specific antigen 2 |
| 129647 | RC_N49394 | AB018259 | Hs.118140 | KIAA0716 gene product |
| 111428 | RC_H56559_s | AL031428 | Hs 174174 | KIAA0601 protein |
| 115967 | RC_AA446887 | AI745379 | Hs.42911 | ESTs |
| 120726 | RC_AA293656 | AA293655 | Hs 97293 | ESTs |
| 114995 | RC_AA251152 | AA769266 | Hs 193657 | ESTs |
| 303876 | RC_AA233334_s | U64820 | Hs.66521 | Machado-Joseph disease (spinocerebellar ataxia 3, |
| | | | | olivopontocerebellar ataxia 3, autosomal dominant, ataxin 3) |
| 311463 | RC_Z39920 | R55344 | Hs.22142 | cytochrome b5 reductase b5R 2 |
| 120302 | RC_AA192173 | AA837098 | Hs.269933 | ESTs |
| 133071 | RC_AA455044 | BE384932 | Hs 64313 | ESTs, Weakly similar to AF257182 1 G-protein-coupled receptor 48 |
| | | | | [H. sapiens] |
| 121032 | RC_AA398504 | AA393037 | Hs.161798 | ESTs |
| 129829 | U41813 | AF010258 | Hs.127428 | homeo box A9 |
| 120245 | RC_AA166965 | AW959615 | Hs 111045 | ESTs |
| 120985 | RC_AA398222 | AI219896 | Hs.97592 | ESTs |
| 114184 | RC_Z39095 | R56434 | Hs 21062 | ESTs |
| 447503 | RC_AA284744_f | AA115496 | Hs.336898 | Homo sapiens, Similar to RIKEN cDNA 1810038N03 gene, clone MGC |
| | | | | 9890, mRNA, complete cds |
| 132837 | RC_AA428201 | AA370362 | Hs 57958 | EGF-TM7-latrophilin-related protein |
| 121034 | RC_AA398507 | AL389951 | Hs.271623 | nucleoporin 50 kD |
| 119718 | RC_W69216 | W69216 | Hs.92848 | ESTs |
| 120455 | RC_AA251083 | AA251720 | Hs.104347 | ESTs, Weakly similar to ALUC_HUMAN !!!! ALU CLASS C |
| | | | | WARNING ENTRY !!! [H. sapiens] |
| 125280 | RC_W93659 | AI123705 | Hs.106932 | ESTs |
| 132155 | RC_AA227903 | AK001607 | Hs.41127 | hypothetical protein FLJ13220 |
| 120609 | RC_AA283902 | AW978721 | Hs 266076 | ESTs, Weakly similar to A46010 X-linked retinopathy protein |
| | | | | [H. sapiens] |
| 121278 | RC_AA401631 | AA037121 | Hs 98518 | Homo sapiens cDNA FLJ11490 fis, clone HEMBA1001918 |
| 109023 | RC_AA157293 | AA57293 | Hs.72168 | ESTs |
| 129815 | RC_D60208_f | BE565817 | Hs 26498 | hypothetical protein FLJ21657 |
| 108061 | RC_AA043979 | AA043979 | Hs.62651 | EST |
| 113287 | RC_T66847 | T66847 | Hs.194040 | ESTs, Weakly similar to I38022 hypothetical protein |
| | | | | [H. sapiens] |
| 114082 | RC_Z38239 | AK001612 | Hs 26962 | Homo sapiens cDNA FLJ10750 fis, clone NT2RP3001929 |
| 116334 | RC_AA491457 | AL038450 | Hs.48948 | ESTs |
| 131486 | RC_Z40071_s | F06972 | Hs 27372 | BMX non-receptor tyrosine kinase |
| 107860 | RC_AA024961 | AA024961 | Hs.50730 | ESTs |
| 131263 | RC_AA443826 | AU077002 | Hs.24950 | regulator of G-protein signalling 5 |
| 132207 | RC_AA443294 | BE206939 | Hs.42287 | E2F transcription factor 6 |
| 129183 | RC_AA155743 | BE561824 | Hs 273369 | uncharacterized hematopoietic stem/progenitor cells protein MDS027 |
| 408431 | RC_T23708 | AI338631 | Hs.43266 | Homo sapiens cDNA: FLJ22536 fis, clone HRC13155 |
| 120575 | RC_AA280934 | AW978022 | Hs.238911 | hypothetical protein DKFZp762E1511; KIAA1816 protein |
| 132121 | RC_AA443284_s | NM_004529 | Hs.404 | myeloid/lymphoid or mixed-lineage leukemia (trithorax (Drosophila) |
| | | | | homolog); translocated to, 3 |
| 117657 | RC_N39074 | N39074 | Hs.44933 | ESTs |
| 134922 | RC_W04507_s | AI718295 | Hs.91161 | prefoldin 4 |
| 118523 | RC_R41828_s | Y07759 | Hs.170157 | myosin VA (heavy polypeptide 12, myoxin) |
| 116845 | RC_H64973 | AA649530 | | gb: ns44f05.s1 NCI_CGAP_Alv1 Homo sapiens cDNA clone, mRNA sequence |
| 115291 | RC_AA279943 | BE545072 | Hs.122579 | hypothetical protein FLJ10461 |
| 120326 | RC_AA196300 | AA196300 | Hs 21145 | hypothetical protein RG083M05 2 |
| 130174 | M29550 | M29551 | Hs 151531 | protein phosphatase 3 (formerly 2B), catalytic subunit, beta |
| | | | | isoform (calcineurin A beta) |
| 129131 | RC_AA436489 | AB026436 | Hs 177534 | dual specificity phosphatase-10 |
| 129868 | RC_AA287032 | AW172431 | Hs.13012 | ESTs |
| 118661 | RC_N70777 | AL137554 | Hs.49927 | protein kinase NYD-SP15 |
| 129829 | RC_AA496921 | AF010258 | Hs 127428 | homeo box A9 |
| 115985 | RC_AA447709 | AA447709 | Hs.268115 | ESTs, Weakly similar to T08599 probable transcription factor CA150 |
| | | | | [H. sapiens] |
| 134637 | RC_AA369856_s | U87309 | Hs 180941 | vacuolar protein sorting 41 (yeast homolog) |
| 132714 | RC_AA252598 | W39388 | Hs 55336 | Homo sapiens, clone MGC: 17421, mRNA, complete cds |
| 129771 | RC_H73237 | AL096748 | Hs.102708 | DKFZP434A043 protein |
| 123360 | RC_AA504784 | AA532718 | Hs.178604 | ESTs |
| 132902 | RC_AA490969 | AI936442 | Hs 59838 | hypothetical protein FLJ10808 |
| 113716 | RC_T97750 | AA001356 | Hs 18159 | ESTs |
| 113825 | RC_W48860 | AW014486 | Hs.22509 | ESTs |
| 130367 | RC_Z38501 | AL135301 | Hs.8768 | hypothetical protein FLJ10849 |
| 120541 | RC_AA278298 | W07318 | Hs.240 | M-phase phosphoprotein 1 |
| 116727 | RC_F13684 | R76472 | Hs.65646 | ESTs |
| 118219 | RC_N62231 | AA862391 | Hs 48494 | ESTs, Moderately similar to A46010 X-linked retinopathy protein |
| | | | | [H. sapiens] |
| 119767 | RC_W72562 | W72562 | Hs 58119 | ESTs |
| 128917 | RC_AA481252 | AI365215 | Hs 206097 | oncogene TC21 |
| 451553 | RC_AA020928 | AA018454 | Hs 269211 | ESTs |
| 132716 | RC_AA251288 | BE379595 | Hs.283738 | casein kinase 1, alpha 1 |
| 118525 | RC_N67861 | N67861 | Hs.49390 | ESTs |
| 114618 | RC_AA084162 | AW979261 | Hs 291993 | ESTs |
| 119743 | RC_W70242 | AA947552 | Hs.58086 | ESTs |
| 108154 | RC_AA425151_s | NM_005754 | Hs 220689 | Ras-GTPase-activating protein SH3-domain-binding protein |
| 122798 | RC_AA460324 | AW366286 | Hs.145696 | splicing factor (CC1.3) |
| 133746 | U44378 | AW410035 | Hs.75862 | MAD (mothers against decapentaplegic, Drosophila) homolog 4 |
| 119822 | RC_W74471 | AF086409 | Hs 301327 | ESTs |
| 122186 | RC_AA435842 | AA398811 | Hs.104673 | ESTs |
| 114941 | RC_AA243017 | AA236512 | Hs 87331 | ESTs |
| 118053 | RC_N53367 | N53391 | Hs.47629 | ESTs |
| 123234 | RC_AA490227 | NM_001938 | Hs.16697 | down-regulator of transcription 1, TBP-binding (negative |
| | | | | cofactor 2) |
| 129280 | M63154 | M63154 | Hs.110014 | gastric intrinsic factor (vitamin B synthesis) |
| 118995 | RC_N94591 | N94591 | Hs.323056 | ESTs |
| 116750 | RC_H05960 | AA760689 | Hs.92418 | ESTs |
| 129026 | M98833 | AL120297 | Hs 108043 | Friend leukemia virus integration 1 |
| 105127 | RC_AA158132 | AA045648 | Hs.301957 | nudix (nucleoside diphosphate linked moiety X)-type motif 5 |
| 114513 | RC_AA044825 | AA044873 | Hs.103446 | ESTs |
| 411856 | RC_T35697 | H67899 | Hs.4190 | Homo sapiens cDNA: FLJ23269 fis, clone COL09533 |
| 132036 | W01568 | AL157433 | Hs 37706 | hypothetical protein DKFZp434E2220 |
| 130091 | RC_W88999 | W88999 | | gb: zh70h03 s1 Soares_fetal_liver_spleen_1NFLS_S1 Homo sapiens cDNA |
| | | | | clone 3′, mRNA sequence |
| 414108 | U09564 | AI267592 | Hs.75761 | SFRS protein kinase 1 |
| 119881 | RC_W81456 | W81486 | Hs 58648 | ESTs |
| 117770 | RC_N47953 | AW957372 | Hs.46791 | ESTs, Weakly similar to I38022 hypothetical protein |
| | | | | [H. sapiens] |
| 119850 | RC_W80447 | AI247568 | Hs.58452 | ESTs |
| 115439 | RC_AA284561 | AI567972 | Hs.193090 | ESTs, Highly similar to AF161437 1 HSPC319 [H. sapiens] |
| 123107 | RC_AA486071 | AA225048 | Hs.104207 | ESTs |
| 406698 | M24364 | X03068 | Hs 73931 | major histocompatibility complex, class II, DQ beta 1 |
| 121231 | RC_AA400780 | AA814948 | Hs 96343 | ESTs, Weakly similar to ALUC_HUMAN !!!! ALU CLASS C |
| | | | | WARNING ENTRY !!! [H sapiens] |
| 132074 | AB002366 | AA478486 | Hs 3852 | KIAA0368 protein |
| 413670 | AB000115 | AB000115 | Hs.75470 | hypothetical protein, expressed in osteoblast |
| 125277 | RC_W93227 | W93227 | Hs.103245 | EST |
| 114056 | RC_AA186324 | AA188175 | Hs 82506 | KIAA1254 protein |
| 121153 | RC_AA399640 | AA399640 | Hs.97694 | ESTs |
| 121609 | RC_AA416867 | AA416867 | Hs 98185 | EST |
| 120661 | RC_AA287556 | AA287556 | Hs.263412 | ESTs, Weakly similar to ALUB_HUMAN !!!! ALU CLASS B |
| | | | | WARNING ENTRY !!! [H. sapiens] |
| 120850 | RC_AA349647 | AA349647 | Hs.96927 | Homo sapiens cDNA FLJ12573 fis, clone NT2RM4000979 |
| 124947 | RC_T03170 | T03170 | Hs.100165 | ESTs |
| 130529 | RC_AA280886 | AA178953 | | gb: zp39e03.s1 Stratagene muscle 937209 Homo sapiens cDNA clone |
| | | | | 3′ similar to contains Alu repetitive element;, mRNA sequence |
| 117683 | RC_N40180 | N40180 | | gb: yy44d02.s1 Soares_multiple_sclerosis_2NbHMSP Homo sapiens cDNA |
| | | | | clone IMAGE 276387 3′ similar to contains L1.t1 L1 repetitive |
| | | | | element;, mRNA sequence. |
| 120745 | RC_AA302809 | AA302809 | | gb: EST10426 Adipose tissue, white I Homo sapiens cDNA 3′ end, |
| | | | | mRNA sequence. |
| 120936 | RC_AA385934 | AA385934 | Hs.97184 | EST, Highly similar to (defline not available 7499603) |
| | | | | [C. elegans] |
| 112597 | RC_R78376 | R78376 | Hs.29733 | EST |
| 120183 | RC_Z40174 | AW082866 | Hs.65882 | ESTs |
| 120644 | RC_AA287038 | AI869129 | Hs.96616 | ESTs |
| 119023 | RC_N98488 | N98488 | | gb: zb82h01 s1 Soares_senescent_fibroblasts_NbHSF Homo sapiens cDNA |
| | | | | clone IMAGE: 310129 3′, mRNA sequence. |
| 107582 | RC_AA002147 | AA002147 | Hs.59952 | EST |
| 118249 | RC_N62580 | N62580 | Hs.322925 | EST, Weakly similar to putative p150 [H. sapiens] |
| 115022 | RC_AA252029 | AA252029 | Hs.87935 | ESTs |
| 117710 | RC_N45198 | N45198 | Hs.47248 | ESTs, Highly similar to similar to Cdc14B1 phosphatase |
| | | | | [H. sapiens] |
| 115341 | RC_AA281452 | AA281452 | Hs.88840 | EST, Weakly similar to granule cell marker protein |
| | | | | [M. musculus] |
| 118896 | RC_N90680 | N46213 | Hs.54642 | methionine adenosyltransferase II, beta |
| 121121 | RC_AA399371 | AA399371 | Hs.189095 | similar to SALL1 (sal (Drosophila)-like |
| 118329 | RC_N63520 | N63520 | | gb: yy62f01 s1 Soares_multiple_sclerosis_2NbHMSP Homo sapiens cDNA |
| | | | | clone IMAGE: 278137 3′, mRNA sequence. |
| 119496 | RC_W35416 | W35416 | Hs.156861 | ESTs, Moderately similar to A46010 X-linked retinopathy protein |
| | | | | [H sapiens] |
| 118111 | RC_N55493 | N55493 | | gb: yv50c02.s1 Soares fetal liver spleen 1NFLS Homo sapiens cDNA |
| | | | | clone IMAGE: 246146 3′, mRNA sequence. |
| 119062 | RC_R16698 | AW444881 | Hs.77829 | ESTs |
| 116710 | RC_F10577_f | F10577 | Hs.306088 | v-crk avian sarcoma virus CT10 oncogene homolog |
| 119261 | RC_T15956 | T15956 | Hs.65289 | EST |
| 122723 | RC_AA457380 | AA457380 | | gb: aa86b10.s1 Stratagene fetal retina 937202 Homo sapiens cDNA |
| | | | | clone IMAGE: 838171 3′ similar to contains L1.b3 L1 repetitive |
| | | | | element;, mRNA sequence |
| 117732 | RC_N46452 | N46452 | | gb: yy76h09.s1 Soares_multiple_sclerosis_2NbHMSP Homo sapiens cDNA |
| | | | | clone IMAGE: 279521 3′ similar to contains L1.t2 L1 repetitive |
| | | | | element;, mRNA sequence |
| 104787 | RC_AA027317 | AA027317 | | gb: ze97d11.s1 Soares_fetal_heart_NbHH19W Homo sapiens cDNA clone |
| | | | | IMAGE: 366933 3′ similar to contains Alu repetitive element;, |
| | | | | mRNA sequence |
| 100071 | A28102 | A28102 | | Human GABAa receptor alpha-3 subunit |
| 115819 | RC_AA426573 | AA486620 | Hs.41135 | endomucin-2 |
| 130882 | RC_Z40166_f | AA497044 | Hs 20887 | hypothetical protein FLJ10392 |
| 125225 | RC_W76540 | W74169 | Hs.16492 | DKFZP564G2022 protein |
| 108339 | RC_AA070801 | AW151340 | Hs.51615 | ESTs, Weakly similar to ALU7_HUMAN ALU SUBFAMILY SQ SEQUENCE |
| | | | | CONTAMINATION WARNING ENTRY [H. sapiens] |
| 100338 | D63483 | D86864 | Hs.57735 | acetyl LDL receptor, SREC |
| 121636 | RC_AA417027 | AA379203 | Hs.306654 | Homo sapiens cDNA FLJ13574 fis, clone PLACE1008625 |
| 103875 | RC_AA418387 | T26379 | Hs.48802 | Homo sapiens clone 23632 mRNA sequence |
| 118716 | RC_N73460 | AI658908 | Hs.118722 | fucosyltransferase 8 (alpha (1,6) fucosyltransferase) |
| 119763 | RC_W72450 | R54146 | Hs.10450 | Homo sapiens cDNA: FLJ22063 fis, clone HEP10326 |
| 121917 | RC_AA428218 | AA406397 | Hs.98038 | ESTs |
| 132806 | M91488 | AI699432 | Hs.278619 | hypothetical protein FLJ10099 |
| 130949 | Y10659 | AV656840 | Hs.285115 | interleukin 13 receptor, alpha 1 |
| 108806 | RC_AA129933 | AF070578 | Hs.71168 | Homo sapiens clone 24674 mRNA sequence |
| 133276 | RC_AA490478 | AW978439 | Hs 69504 | ESTs |
| 134760 | RC_H16758 | NM_000121 | Hs.89548 | erythropoietin receptor |
| 132867 | AA121287 | AF226667 | Hs 58553 | CTP synthase II |
| 132051 | AA091284 | AA393968 | Hs.180145 | HSPC030 protein |
| 114208 | RC_Z39301 | AL049466 | Hs.7859 | ESTs |
| 104094 | AA418187 | AA418187 | Hs.330515 | ESTs |
| 128718 | AA426361 | NM_002959 | Hs.281706 | sortilin 1 |
| 302032 | RC_N20407 | NM_001992 | Hs.128087 | coagulation factor II (thrombin) receptor |
| 115501 | RC_AA291553 | AA291553 | Hs.190086 | ESTs |
| 101997 | U01160 | AU076536 | Hs.50984 | sarcoma amplified sequence |
| 103708 | AA037206 | AA430591 | Hs.72071 | hypothetical protein FLJ20038 |
| 101899 | S59184 | S59184 | Hs.79350 | RYK receptor-like tyrosine kinase |
| 115839 | RC_AA429038 | BE300266 | Hs 28935 | transducin-like enhancer of split 1, homolog of Drosophila E(sp1) |
| 409459 | D50678 | D86407 | Hs.54481 | low density lipoprotein receptor-related protein 8, apolipoprotein |
| | | | | e receptor |
| 103563 | Z22534 | L02911 | Hs.150402 | Activin A receptor, type I (ACVR1) (ALK-2) |
| 123233 | RC_AA490225 | AW974175 | Hs 188751 | ESTs, Weakly similar to MAPB_HUMAN MICROTUBULE-ASSOCIATED |
| | | | | PROTEIN 1B [H. sapiens] |
| 121305 | RC_AA402468 | AA402468 | Hs.291557 | ESTs |
| 114798 | RC_AA159181 | AA159181 | Hs.54900 | serologically defined colon cancer antigen 1 |
| 133145 | RC_AA196549 | H94227 | Hs.6592 | Homo sapiens, clone IMAGE: 2961368, mRNA, partial cds |
| 131567 | RC_AA291015_s | AF015592 | Hs 28853 | CDC7 (cell division cycle 7, S. cerevisiae, homolog)-like 1 |
| 112300 | RC_R54554 | H24334 | Hs.26125 | ESTs |
| 129507 | RC_AA192099 | AJ236885 | Hs 112180 | zinc finger protein 148 (pHZ-52) |
| 121033 | RC_AA398505 | AA398505 | Hs.97360 | ESTs |
| 121151 | RC_AA399636 | AA399636 | Hs.143629 | ESTs |
| 121402 | RC_AA406063 | AA406063 | Hs.98003 | ESTs |
| 123203 | RC_AA489671 | AA352335 | Hs.65641 | hypothetical protein FLJ20073 |
| 132271 | RC_AA236466 | AB030034 | Hs.115175 | sterile-alpha motif and leucine zipper containing kinase AZK |
| 125197 | RC_W69106 | AF086270 | Hs.278554 | heterochromatin-like protein 1 |
| 114935 | RC_AA242809 | H23329 | Hs 290880 | ESTs, Weakly similar to ALU1_HUMAN ALU SUBFAMILY J SEQUENCE |
| | | | | CONTAMINATION WARNING ENTRY [H. sapiens] |
| 125279 | RC_W93640 | AW401809 | Hs.4779 | KIAA1150 protein |
| 108778 | RC_AA128548 | AF133123 | Hs.90847 | general transcription factor IIIC, polypeptide 3 (102 kD) |
| 108087 | RC_AA045709 | AA045708 | Hs.40545 | ESTs |
| 132466 | RC_N66810_s | AI597655 | Hs.49265 | ESTs |
| 133328 | R36553 | AW452738 | Hs.265327 | hypothetical protein DKFZp761l141 |
| 124057 | RC_F13604 | AA902384 | Hs.73853 | bone morphogenetic protein 2 |
| 124800 | RC_R45115 | AW864086 | Hs.138617 | thyroid hormone receptor interactor 12 |
| 121029 | RC_AA398482 | AA398482 | Hs 97641 | EST |
| 120663 | RC_AA287627 | AA827798 | Hs.105089 | ESTs |
| 102133 | U15173 | AU076845 | Hs.155596 | BCL2/adenovirus E1B 19 kD-interacting protein 2 |
| 108246 | RC_AA062855 | AI423132 | Hs.146343 | ESTs |
| 125226 | RC_W78134 | AA782536 | Hs.122647 | N-mynstoyltransferase 2 |
| 120260 | RC_AA171739 | AK000061 | Hs.101590 | hypothetical protein |
| 124906 | RC_R87647 | H75964 | Hs.107815 | ESTs |
| 109406 | RC_AA226877 | AA199883 | Hs.67624 | ESTs |
| 109271 | RC_AA195668 | AW137422 | Hs.86022 | ESTs |
| 125052 | RC_T80174_s | T85104 | Hs.222779 | ESTs, Moderately similar to similar to NEDD-4 [H. sapiens] |
| 109101 | RC_AA167708 | AW608930 | Hs.52184 | hypothetical protein FLJ20618 |
| 115241 | RC_AA278723 | AA648278 | Hs.193859 | ESTs |
| 117163 | RC_H97909 | N36861 | Hs.42344 | ESTs |
| 113530 | RC_T90313 | T90313 | Hs.16732 | ESTs |
| 120375 | RC_AA227260 | AF028706 | Hs.111227 | Zic family member 3 (odd-paired Drosophila homolog, heterotaxy 1) |
| 129435 | AA314256 | AF151852 | Hs.111449 | CGI-94 protein |
| 114864 | RC_AA235256 | AA135332 | Hs.71608 | ESTs |
| 103988 | AA314389 | AA314389 | Hs.42500 | ADP-ribosylation factor-like 5 |
| 131006 | RC_AA242763 | AF064104 | Hs 22116 | CDC14 (cell division cycle 14, S. cerevisiae) homolog B |
| 106781 | RC_AA478474 | AA330310 | Hs.24181 | ESTs |
| 106141 | RC_AA424558 | AF031463 | Hs.9302 | phosducin-like |
| 116213 | RC_AA476738 | AA292105 | Hs.326740 | hypothetical protein MGC10947 |
| 135266 | AB002326 | R41179 | Hs.97393 | KIAA0328 protein |
| 135058 | RC_AA430152 | AI379720 | Hs.93814 | hypothetical protein |
| 119908 | RC_W85844 | AA524470 | Hs.58753 | ESTs |
| 103695 | AA018758 | AW207152 | Hs 186600 | ESTs |
| 103978 | AA307443 | NM_016940 | Hs.34136 | chromosome 21 open reading frame 6 |
| 109485 | RC_AA233472 | BE619092 | Hs.28465 | Homo sapiens cDNA: FLJ21869 fis, clone HEP02442 |
| 129574 | AA458603 | AA026815 | Hs.11463 | UMP-CMP kinase |
| 115347 | RC_AA281528 | AA356792 | Hs.334824 | hypothetical protein FLJ14825 |
| 120765 | RC_AA338735 | AW961026 | Hs.96752 | ESTs, Weakly similar to ALU8_HUMAN ALU SUBFAMILY SX SEQUENCE |
| | | | | CONTAMINATION WARNING ENTRY [H. sapiens] |
| 121059 | RC_AA398628 | AA393283 | | gb: zt74e03 r1 Soares_testis_NHT Homo sapiens cDNA clone 5′, |
| | | | | mRNA sequence |
| 131887 | AA046548 | W17064 | Hs 332848 | SWI/SNF related, matrix associated, actin dependent regulator of |
| | | | | chromatin, subfamily e, member 1 |
| 112064 | RC_R43812 | AL049390 | Hs.22689 | Homo sapiens mRNA; cDNA DKFZp586O1318 (from clone DKFZp586O1318) |
| 115606 | RC_AA400465 | AI025829 | Hs.86320 | ESTs |
| 131750 | RC_H94855_s | NM_004349 | Hs 31551 | core-binding factor, runt domain, alpha subunit 2; translocated |
| | | | | to, 1; cyclin D-related |
| 102123 | U14518 | NM_001809 | Hs.1594 | centromere protein A (17 kD) |
| 129847 | RC_W46767 | N64025 | Hs 296178 | hypothetical protein FLJ22637 |
| 133809 | RC_AA235275 | AV649326 | Hs 76359 | catalase |
| 132210 | RC_N51499_s | NM_007203 | Hs 42322 | A kinase (PRKA) anchor protein 2 |
| 122356 | RC_AA443794 | AA443794 | Hs 98390 | ESTs |
| 114958 | RC_AA243708 | N20912 | Hs.42369 | ESTs |
| 103951 | AA287840 | AL353944 | Hs 50115 | Homo sapiens mRNA; cDNA DKFZp761J1112 (from clone DKFZp761J1112) |
| 134703 | RC_AA280704 | AF117065 | Hs.88764 | male-specific lethal-3 (Drosophila)-like 1 |
| 128727 | AA287864 | AI223335 | Hs 50651 | Janus kinase 1 (a protein tyrosine kinase) |
| 105743 | RC_AA293300_s | BE246502 | Hs.9598 | sema domain, immunoglobulin domain (Ig), transmembrane domain (TM) |
| | | | | and short cytoplasmic domain, (semaphorin) 4B |
| 103744 | AA076003 | AA079267 | | gb: zm97e10.s1 Stratagene colon HT29 (937221) Homo sapiens cDNA |
| | | | | clone 3′, mRNA sequence |
| 114348 | N80402 | AL050321 | Hs 301532 | CRP2 binding protein |
| 114009 | RC_W90067 | AI248544 | Hs.103000 | KIAA0831 protein |
| 134704 | RC_AA280849 | AA837124 | Hs.88780 | ESTs |
| 128629 | AA399187 | AL096748 | Hs.102708 | DKFZP434A043 protein |
| 104410 | H65925 | AI807519 | Hs.104520 | Homo sapiens cDNA FLJ13694 fis, clone PLACE2000115 |
| 110200 | RC_H21075 | H21075 | Hs 31802 | ESTs, Highly similar to A59266 unconventional myosin-15 |
| | | | | [H sapiens] |
| 124483 | RC_N53976 | AI821780 | Hs.179864 | ESTs |
| 101391 | M14648 | NM_002210 | Hs 295726 | integrin, alpha V (vitronectin receptor, alpha polypeptide, |
| | | | | antigen CD51) |
| 109657 | RC_F04826 | R60900 | Hs.26814 | ESTs |
| 117140 | RC_H96813 | H96813 | Hs.42241 | ESTs |
| 132937 | RC_AA233706_f | AW952912 | Hs 300383 | hypothetical protein MGC3032 |
| 129799 | R36410 | AW967473 | Hs.239114 | mannosidase, alpha, class 1A, member 2 |
| 105077 | RC_AA142919 | W55946 | Hs 234863 | Homo sapiens cDNA FLJ12082 fis, clone HEMBB1002492 |
| 100850 | RC_N58561_s | AA836472 | Hs.297939 | cathepsin B |
| 131043 | RC_AA490925 | AF084535 | Hs.22464 | epilepsy, progressive myoclonus type 2, Lafora disease (laforin) |
| 118417 | RC_N66048_f | AF080229 | | gb: Human endogenous retrovirus K clone 10.1 polymerase mRNA, |
| | | | | partial cds |
| 129254 | RC_AA243695 | AA252468 | Hs.1098 | DKFZp434J1813 protein |
| 119149 | RC_R58910 | BE304701 | Hs.65732 | ESTs |
| 133996 | AA091367 | AA380267 | Hs.78277 | DKFZP434F2021 protein |
| 110223 | RC_H23747 | H19836 | Hs 31697 | ESTs |
| 117626 | RC_N36090 | AK001757 | Hs.281348 | hypothetical protein FLJ10895 |
| 135286 | RC_AA424469_s | AW023482 | Hs.97849 | ESTs |
| 122967 | RC_AA478521 | AA806187 | Hs.289101 | glucose regulated protein, 58 kD |
| 131236 | AA282640 | AF043117 | Hs.24594 | ubiquitination factor E4B (homologous to yeast UFD2) |
| 128568 | AA463380 | H12912 | Hs.274691 | adenylate kinase 3 |
| 112888 | RC_T03872 | AW195317 | Hs 107716 | hypothetical protein FLJ22344 |
| 115192 | RC_AA261920 | AA741024 | Hs.88378 | ESTs |
| 118688 | RC_N71484 | AK000708 | Hs.169764 | hypothetical protein FLJ20701 |
| 122264 | RC_AA436837 | AA436837 | | gb: zv57g07.s1 Soares_testis_NHT Homo sapiens cDNA clone 3′, |
| | | | | mRNA sequence |
| 128981 | AA135452 | AA927177 | Hs.86041 | CGG triplet repeat binding protein 1 |
| 131042 | RC_R42457 | AI826288 | Hs.171637 | hypothetical protein MGC2628 |
| 103704 | AA028171 | AA028171 | Hs.151258 | hypothetical protein FLJ21062 |
| 121341 | AA233107 | AF035528 | Hs.153863 | MAD (mothers against decapentaplegic, Drosophila) homolog 6 |
| 106593 | RC_AA456826 | AW296451 | Hs 24605 | ESTs |
| 115195 | RC_AA262156 | AW968619 | Hs.155849 | ESTs |
| 115425 | RC_AA284071 | AA811895 | Hs 180680 | ESTs, Weakly similar to I54374 gene NF2 protein [H. sapiens] |
| 117258 | RC_N21299 | AF086041 | Hs.42975 | ESTs |
| 120209 | RC_Z40892 | F02951 | | gb: HSC1HB082 normalized infant brain cDNA Homo sapiens cDNA clone |
| | | | | c-1hb08 3′, mRNA sequence |
| 134082 | L16991 | L16991 | Hs.79006 | deoxythymidylate kinase (thymidylate kinase) |
| 104774 | RC_AA026066 | AW959755 | Hs 288896 | Homo sapiens cDNA FLJ12977 fis, clone NT2RP2006261 |
| 115625 | RC_AA401630 | AA059459 | Hs 62592 | ESTs |
| 104469 | N28707 | N28707 | Hs.154304 | Homo sapiens chromosome 19, BAC 282485 (CIT-B-344H19) |
| 107401 | W20054 | N91453 | Hs.102987 | ESTs |
| 111686 | RC_R21510 | R22039 | Hs.23217 | ESTs |
| 115300 | RC_AA280026 | AA280095 | Hs.88689 | ESTs |
| 115378 | RC_AA282292 | AA282292 | Hs.279841 | hypothetical protein FLJ10335 |
| 132224 | RC_H97819 | N41549 | Hs 285410 | ESTs |
| 113791 | M95767 | AI269096 | Hs 135578 | chitobiase, di-N-acetyl- |
| 129144 | AA004987 | AL137275 | Hs.20137 | hypothetical protein DKFZp434P0116 |
| 104448 | L44574 | NM_007331 | Hs.110457 | Wolf-Hirschhorn syndrome candidate 1 |
| 132084 | RC_T26981_s | NM_002267 | Hs 3886 | karyopherin alpha 3 (importin alpha 4) |
| 111831 | RC_R36083 | R36095 | Hs.268695 | ESTs |
| 114765 | RC_AA252163 | AA463550 | Hs.337532 | ESTs, Weakly similar to A47582 B-cell growth factor precursor |
| | | | | [H. sapiens] |
| 115029 | RC_AA252219 | AL137939 | Hs.40096 | ESTs |
| 100457 | H81492 | BE246400 | Hs.285176 | acetyl-Coenzyme A transporter |
| 104536 | R24011 | R24024 | Hs.158101 | Homo sapiens cDNA FLJ14673 fis, clone NT2RP2003714, moderately |
| | | | | similar to ZINC FINGER PROTEIN 91 |
| 116167 | RC_AA461562 | AI091731 | Hs.87293 | hypothetical protein FLJ20045 |
| 103889 | AA236771 | R85350 | Hs.101368 | ESTs |
| 131978 | RC_H48459_s | AA355925 | Hs.36232 | KIAA0186 gene product |
| 118843 | RC_N80181 | N80181 | Hs.221498 | ESTs |
| 120837 | RC_W93092 | BE149656 | Hs.306621 | Homo sapiens cDNA FLJ11963 fis, clone HEMBB1001051 |
| 133647 | D21852 | NM_015361 | Hs.268053 | KIAA0029 protein |
| 129521 | U41815 | AF071076 | Hs.112255 | nucleoporin 98 kD |
| 103746 | AA081876 | AA075000 | | gb: zm83c07.s1 Stratagene ovarian cancer (937219) Homo sapiens cDNA |
| | | | | clone 3′, mRNA sequence |
| 132019 | RC_AA134965_i | H56995 | Hs.37372 | Homo sapiens DNA binding peptide mRNA, partial cds |
| 132310 | RC_AA284107 | AA173223 | Hs.289044 | Homo sapiens cDNA FLJ12048 fis, clone HEMBB1001990 |
| 117367 | RC_N24954 | AI041793 | Hs 42502 | ESTs |
| 103743 | AA075998 | AA075998 | | gb: zm89b09.r1 Stratagene ovarian cancer (937219) Homo sapiens cDNA |
| | | | | clone 5′ similar to gb: M15887 ACYL-COA-BINDING PROTEIN |
| | | | | (HUMAN);, mRNA sequence |
| 103761 | AA085138 | AA765163 | | gb: nz79b10.s1 NCI_CGAP_GCB1 Homo sapiens cDNA clone 3′ similar |
| | | | | to gb: M34539 FK506-BINDING PROTEIN (HUMAN);, mRNA sequence |
| 130237 | L39060 | AA913909 | Hs.153088 | TATA box binding protein (TBP)-associated factor, RNA |
| | | | | polymerase I, A, 48 kD |
| 128752 | RC_N72879 | AA504428 | Hs 10487 | Homo sapiens, clone IMAGE: 3954132, mRNA, partial cds |
| 135162 | AA045930 | AI187925 | Hs.95667 | F-box protein 30 |
| 131386 | AA096412 | BE219898 | Hs.173135 | dual-specificity tyrosine-(Y)-phosphorylation regulated kinase 2 |
| 129021 | RC_AA599244 | AL044675 | Hs 173081 | KIAA0530 protein |
| 424274 | AA293634 | W73933 | Hs.283738 | casein kinase 1, alpha 1 |
| 129913 | H06583 | NM_001310 | Hs.13313 | cAMP responsive element binding protein-like 2 |
| 131888 | U79298 | AW294659 | Hs.34054 | Homo sapiens cDNA: FLJ22488 fis, clone HRC10948, highly similar to |
| | | | | HSU79298 Human clone 23803 mRNA |
| 118612 | RC_N69466 | AB037788 | Hs.224961 | cleavage and polyadenylation specific factor 2, 100 kD subunit |
| 322026 | AA203138 | AW024973 | Hs.283675 | NPD009 protein |
| 110892 | RC_N38882 | AL035301 | Hs.97375 | H. sapiens gene from PAC 106H8 |
| 111429 | RC_R01245 | AI038052 | Hs.19162 | ESTs, Weakly similar to I54374 gene NF2 protein [H sapiens] |
| 113334 | RC_T76962 | AW974666 | Hs.293024 | ESTs |
| 104091 | AA417310 | BE465093 | Hs.106101 | hypothetical protein FLJ22557 |
| 105246 | RC_AA226879 | AA226879 | | gb: zr19c09.s1 Stratagene NT2 neuronal precursor 937230 |
| | | | | Homo sapiens cDNA clone IMAGE: 663856 3′ similar to contains |
| | | | | Alu repetitive element;, mRNA sequence. |
| 113300 | RC_T67448 | T67448 | Hs.13101 | ESTs |
| 117147 | RC_H97225_s | AW901347 | Hs.38592 | hypothetical protein FLJ23342 |
| 121349 | RC_AA405205 | AA405205 | Hs 97960 | ESTs, Weakly similar to T51146 ring-box protein 1 |
| | | | | [H. sapiens] |
| 100294 | D49396 | AA331881 | Hs.75454 | peroxiredoxin 3 |
| 133999 | M28213 | AA535244 | Hs.78305 | RAB2, member RAS oncogene family |
| 133259 | AA278548 | BE379646 | Hs.6904 | Homo sapiens mRNA full length insert cDNA clone EUROIMAGE 2004403 |
| 129423 | AA371418 | AA204686 | Hs.234149 | hypothetical protein FLJ20647 |
| 131098 | RC_AA459668 | U66669 | Hs.236642 | 3-hydroxyisobutyryl-Coenzyme A hydrolase |
| 135272 | AA399391 | AI828337 | Hs.97591 | ESTs |
| 129155 | AA046865 | AI952677 | Hs.108972 | Homo sapiens mRNA; cDNA DKFZp434P228 (from clone DKFZp434P228) |
| 311291 | AA056319 | AA782601 | Hs.319817 | ESTs |
| 120750 | RC_AA310499 | AI191410 | Hs 96693 | ESTs, Moderately similar to 2109260A B cell growth factor |
| | | | | [H. sapiens] |
| 101002 | J04058 | AV655843 | Hs.169919 | electron-transfer-flavoprotein, alpha polypeptide (glutaric |
| | | | | aciduria II) |
| 133012 | AA099241 | AA847843 | Hs.62711 | Homo sapiens, clone IMAGE 3351295, mRNA |
| 103879 | AA228148_s | BE543269 | Hs.50252 | mitochondrial ribosomal protein L32 |
| 131281 | RC_AA443212 | AA251716 | Hs 25227 | ESTs |
| 115109 | RC_AA256383 | AJ249977 | Hs 88049 | protein kinase, AMP-activated, gamma 3 non-catalytic subunit |
| 118502 | RC_N67317 | AL157488 | Hs.50150 | Homo sapiens mRNA; cDNA DKFZp564B182 (from clone DKFZp564B182) |
| 134100 | L07540 | AA460085 | Hs.171075 | replication factor C (activator 1) 5 (36.5 kD) |
| 131869 | AA484944 | AW968547 | Hs.33540 | ESTs, Weakly similar to dJ309K20.4 [H. sapiens] |
| 115396 | RC_AA282985 | AA810854 | Hs.89081 | ESTs |
| 103860 | AA203742 | AW976877 | Hs.38057 | ESTs |
| 135089 | N75611_s | AI918035 | Hs.301198 | roundabout (axon guidance receptor, Drosophila) homolog 1 |
| 129938 | U79300 | AW003668 | Hs.135587 | Human clone 23629 mRNA sequence |
| 107508 | W90095 | N74925 | Hs 38761 | Homo sapiens cDNA. FLJ21564 fis, clone COL06452 |
| 103685 | AA005190 | AA158008 | Hs.292444 | ESTs |
| 125170 | AA203147 | AL020996 | Hs 8518 | selenoprotein N |
| 129179 | RC_AA504125_s | AW969025 | Hs.109154 | ESTs |
| 116262 | AA477046 | AI936442 | Hs 59838 | hypothetical protein FLJ10808 |
| 123009 | RC_AA479949 | AA535244 | Hs.78305 | RAB2, member RAS oncogene family |
| 131004 | D29833 | D29833 | Hs.2207 | salivary proline-rich protein |
| 103317 | X83441 | X83441 | Hs.166091 | ligase IV, DNA, ATP-dependent |
| 132814 | RC_C15251_f | D60730 | Hs.57471 | ESTs |
| 103992 | U77718 | BE018142 | Hs.300954 | Huntingtin interacting protein K |
| 109258 | X59710 | AL044818 | Hs 84928 | nuclear transcription factor Y, beta |
| 110754 | RC_N20814 | AW302200 | Hs.6336 | KIAA0672 gene product |
| 132727 | AA136382_s | N27495 | Hs.5565 | hypothetical protein FLJ22626 |
| 100341 | D63506 | AF032922 | Hs.8813 | syntaxin binding protein 3 |
| 134664 | AA256106 | AA256106 | Hs 87507 | ESTs |
| 103826 | AA165564 | AW162998 | Hs.24684 | KIAA1376 protein |
| 111678 | RC_R20628 | R38487 | Hs.169927 | ESTs |
| 101341 | L76159 | NM_004477 | Hs 203772 | FSHD region gene 1 |
| 115455 | RC_AA285068 | AA876002 | Hs.120551 | toll-like receptor 10 |
| 111192 | RC_AA477748 | AW021968 | Hs.109438 | Homo sapiens clone 24775 mRNA sequence |
| 129385 | RC_AA235604 | AA172106 | Hs.110950 | Rag C protein |
| 125050 | RC_T79951 | AW970209 | Hs.111805 | ESTs |
| 122105 | RC_AA432278 | AW241685 | Hs 98699 | ESTs |
| 121324 | RC_AA404229 | AA404229 | Hs.97842 | EST |
| 120938 | RC_AA386260 | AA386260 | Hs.104632 | EST |
| 115001 | RC_AA251376 | AA251376 | | gb: zs10a06.s1 NCI_CGAP_GCB1 Homo sapiens cDNA clone IMAGE: 684754 |
| | | | | 3′, mRNA sequence. |
| 124799 | RC_R45088 | R45088 | | gb: yg38g04.s1 Soares infant brain 1NIB Homo sapiens cDNA clone |
| | | | | IMAGE: 34896 3′, mRNA sequence. |
| 122724 | RC_AA457395 | AA457395 | Hs.99457 | ESTs |
| 117791 | RC_N48325 | N48325 | Hs.93956 | EST |
| 121895 | RC_AA427396 | AA427396 | | gb: zw33a02 s1 Soares ovary tumor NbHOT Homo sapiens cDNA clone |
| | | | | IMAGE: 771050 3′ similar to contains Alu repetitive element, |
| | | | | contains MER12.t2 MER12 repetitive element;, mRNA sequence. |
| 108244 | RC_AA062839 | AA062839 | | gb: zm05c09.s1 Stratagene corneal stroma (937222) Homo sapiens cDNA |
| | | | | clone IMAGE 513232 3′, mRNA sequence. |
| 117852 | RC_N49408 | AW877787 | Hs 136102 | KIAA0853 protein |
| 109298 | RC_AA205432 | R77854 | Hs.250693 | Krueppel-related zinc finger protein |
| 122432 | RC_AA447400 | AA447400 | Hs.187684 | ESTs, Weakly similar to B34087 hypothetical protein |
| | | | | [H. sapiens] |
| 124627 | RC_N74625 | N74625 | | gb: za55c03.s1 Soares fetal liver spleen 1NFLS Homo sapiens cDNA |
| | | | | clone IMAGE: 296452 3′ similar to gb: M14338 VITAMIN K- |
| | | | | DEPENDENT PROTEIN S PRECURSOR (HUMAN); contains OFR b3 OFR |
| | | | | repetitive element;, mRNA sequence |
| 115141 | RC_AA258071 | AA465131 | Hs.64001 | Homo sapiens clone 25218 mRNA sequence |
| 128636 | U49065 | U49065 | Hs 102865 | interleukin 1 receptor-like 2 |
| 115373 | RC_AA282197 | AA664862 | Hs.181022 | CGI-07 protein |
| 114651 | RC_AA101400 | AA101400 | Hs.189960 | ESTs |
| 132796 | RC_AA180487 | NM_006283 | Hs.173159 | transforming, acidic coiled-coil containing protein 1 |
| 103749 | RC_N35583 | AL135301 | Hs 8768 | hypothetical protein FLJ10849 |
| 107328 | T83444 | AW959891 | Hs.76591 | KIAA0887 protein |
| 115349 | RC_AA281563 | AF121176 | Hs 12797 | DEAD/H (Asp-Glu-Ala-Asp/His) box polypeptide 16 |
| 111490 | RC_R06862 | R06862 | | gb: yf11e09.s1 Soares fetal liver spleen 1NFLS Homo sapiens cDNA |
| | | | | clone IMAGE: 126568 3′ similar to contains L1 repetitive |
| | | | | element;, mRNA sequence. |
| 103763 | AA085354 | AA085291 | | gb: zn01g06.s1 Stratagene colon HT29 (937221) Homo sapiens cDNA |
| | | | | clone 3′ similar to contains Alu repetitive element;, mRNA |
| | | | | sequence. |
| 118791 | RC_N75520 | N75520 | Hs.261003 | ESTs, Moderately similar to B34087 hypothetical protein |
| | | | | [H sapiens] |
| 116644 | RC_F03032 | F03032 | Hs.290278 | ESTs, Weakly similar to B34087 hypothetical protein |
| | | | | [H. sapiens] |
| 116823 | RC_H56485 | AW204742 | Hs 143542 | ESTs, Highly similar to CSA_HUMAN COCKAYNE SYNDROME WD-REPEAT |
| | | | | PROTEIN CSA [H. sapiens] |
| 108940 | RC_AA148603 | AA148603 | | gb: zo09e04 s1 Stratagene neuroepithelium NT2RAMI 937234 |
| | | | | Homo sapiens cDNA clone IMAGE: 567198 3′, mRNA sequence. |
| 112218 | RC_R50057 | R50057 | Hs.272251 | Homo sapiens mRNA; cDNA DKFZp586M1418 (from clone DKFZp586M1418) |
| 116557 | RC_D20572_i | D20572 | Hs 90171 | EST |
| 133649 | U25849 | U25849 | Hs.75393 | acid phosphatase 1, soluble |
| 131745 | RC_C20746 | AI828559 | Hs.31447 | ESTs, Moderately similar to A46010 X-linked retinopathy protein |
| | | | | [H. sapiens] |
| 116801 | RC_H43879 | H43879 | | gb: yo69h09.s1 Soares breast SNbHBst Homo sapiens cDNA clone |
| | | | | IMAGE: 183233 3′, mRNA sequence. |
| 115006 | RC_AA251548 | AA251548 | Hs.87886 | EST |
| 123424 | RC_AA598500 | H29882 | Hs.162614 | ESTs |
| 120831 | RC_AA347919 | AA347919 | Hs.96889 | EST |
| 103691 | AA018298 | AA018298 | Hs.103332 | ESTs |
| 121555 | RC_AA412491 | AF025771 | Hs.50123 | zinc finger protein 189 |
| 111193 | RC_N67946 | N67946 | Hs 117569 | ESTs |
| 132061 | RC_AA058946 | AB020700 | Hs 3830 | KIAA0893 protein |
| 134575 | RC_AA194568J | AA194568 | Hs.85938 | EST |
| 115050 | RC_AA252794 | AA252794 | Hs.88009 | ESTs |
| 420208 | U31799 | BE276055 | Hs.95972 | silver (mouse homolog) like |
| 133735 | AC002045_xpt1 | R66740 | Hs.110613 | KIAA0220 protein |
| 128546 | Z21305 | NM_003478 | Hs.101299 | cullin 5 |
| 111946 | RC_R40697 | R40697 | Hs.76666 | C9orf10 protein |
| 124879 | RC_R73588 | R73588 | Hs.101533 | ESTs |
| 115683 | AA410345 | AF255910 | Hs.54650 | junctional adhesion molecule 2 |
| 103692 | AA018418 | AW137912 | Hs.227583 | Homo sapiens chromosome X map Xp11.23 L-type calcium channel |
| | | | | alpha-1 subunit (CACNA1F) gene, complete cds; HSP27 pseudogene, |
| | | | | complete sequence; and JM1 protein, JM2 protein, and Hb2E genes, |
| | | | | complete cds |
| 103767 | AA089688 | BE244667 | Hs 296155 | CGI-100 protein |
| 125266 | W90022 | W90022 | Hs.186809 | ESTs, Highly similar to LCT2_HUMAN LEUKOCYTE CELL-DERIVED |
| | | | | CHEMOTAXIN 2 PRECURSOR [H sapiens] |
| 135235 | AA435512 | AW298244 | Hs.293507 | ESTs |
| 134497 | RC_AA404494 | BE258532 | Hs.251871 | CTP synthase |
| 426754 | RC_AA278529_i | NM_014264 | Hs.172052 | serine/threonine kinase 18 |
| 412177 | RC_AA342828_s | Z23091 | Hs.73734 | glycoprotein V (platelet) |
| 132000 | RC_AA044644 | AW247017 | Hs 36978 | melanoma antigen, family A, 3 |
| 124738 | RC_AA044644 | T07568 | Hs.137158 | ESTs |
| 324000 | RC_AA196729_i | AA604749 | Hs.190213 | ESTs |
| 106896 | RC_AA196729_i | AW073202 | Hs.334825 | Homo sapiens cDNA FLJ14752 fis, clone NT2RP3003071 |
| 132000 | RC_AA025858 | AW247017 | Hs.36978 | melanoma antigen, family A, 3 |
| 129577 | RC_AA025858 | N75346 | Hs.82906 | CDC20 (cell division cycle 20, S. cerevisiae, homolog) |
| 107091 | RC_AA233519 | AI949109 | Hs 246885 | hypothetical protein FLJ20783 |
| 130296 | RC_N52271 | D31139 | Hs.154103 | LIM protein (similar to rat protein kinase C-binding enigma) |
| 102855 | RC_N68399 | NM_003528 | Hs.2178 | H2B histone family, member Q |
| 113689 | RC_AA098874 | AB037850 | Hs 16621 | DKFZP434I116 protein |
| 100939 | RC_AA279667_s | L04288 | Hs.297939 | cathepsin B |
| 130430 | RC_H22556 | W27893 | Hs 150580 | putative translation initiation factor |
| 106734 | RC_N45979_s | BE296690 | Hs.288173 | Homo sapiens cDNA: FLJ21747 fis, clone COLF5160, highly similar |
| | | | | to AF182198 Homo sapiens intersectin 2 long isoform (ITSN2) mRNA |
| 135148 | RC_AA431288_s | AA306478 | Hs 95327 | CD3D antigen, delta polypeptide (TiT3 complex) |
| 134221 | RC_AA609862 | BE280456 | Hs.80248 | RNA-binding protein gene with multiple splicing |
| 105376 | RC_N35583 | AW994032 | Hs.8768 | hypothetical protein FLJ10849 |
| 124541 | U77718 | AF112222 | Hs.44499 | pinin, desmosome associated protein |
| 134546 | AA203147 | AL020996 | Hs.8518 | selenoprotein N |
| 134000 | RC_W93092 | AW175787 | Hs 334841 | selenium binding protein 1 |
| 125656 | RC_W93092 | AW516428 | Hs.78687 | neutral sphingomyelinase (N-SMase) activation associated factor |
| 100939 | RC_N58561_s | L04288 | Hs.297939 | cathepsin B |
| 125656 | RC_W93092 | AW516428 | Hs.78687 | neutral sphingomyelinase (N-SMase) activation associated factor |
| 101779 | RC_W69385_s | BE543412 | Hs.250505 | retinoic acid receptor, alpha |
| 332489 | RC_R22947 | R23053 | NA | Hu01 Chip Redos |
| 133000 | RC_N38959_f | AL042444 | Hs.62402 | p21/Cdc42/Rac1-activated kinase 1 (yeast Ste20-related) |
| 125905 | RC_N38959_f | AI678638 | Hs.6456 | chaperonin containing TCP1, subunit 2 (beta) |
| 129000 | RC_H73050_s | AA744902 | Hs.107767 | hypothetical protein PRO1489 |
| 100920 | RC_H73050_s | X54534 | Hs.278994 | Rhesus blood group, CcEe antigens |

TABLE 1A

| | |
|---|---|
| Pkey: | Unique Eos probeset identifier number |
| CAT number: | Gene cluster number |
| Accession: | Genbank accession numbers |

| Pkey | CAT Number | Accession |
|---|---|---|
| 108469 | 116761_1 | AA079487 AA128547 AA128291 AA079587 AA079600 |
| 124106 | 125446_1 | H12245 AA094769 R14576 |
| 108501 | 13684_-12 | AA083256 |
| 108562 | 36375_1 | AA100796 AF020589 AA074629 AA075946 AA100849 AA085347 AA126309 |
| | | AA079311 AA079323 AA085274 |
| 125008 | 1802095_1 | T91251 T64891 T85665 |
| 125020 | 116017_1 | T69981 T69924 AA078476 |
| 125066 | 1814993_1 | T86284 T81933 |
| 116661 | 1532859_1 | R61504 F04247 |
| 125104 | 413347_1 | T95590 AA703278 H62764 |
| 124575 | 1666649_1 | N68168 N69188 N90450 |
| 125263 | 1547_2 | AA098878 W88942 |
| 116845 | 393481_1 | AA649530 AA659316 H64973 |
| 118417 | 37186_1 | AF080229 AF080231 AF080230 AF080232 AF080233 AF080234 BE550633 |
| | | AI636743 AW614951 BE467547 AI680833 AI633818 N29986 U87592 U87593 |
| | | U87590 U87591 S46404 U87587 AA463992 AW206802 AI970376 AI583718 |
| | | AI672574 N25695 AW665466 AI818326 AA126128 AI480345 AW013827 |
| | | AA248638 AI214968 AA204735 AA207155 AA206262 AA204833 AW003247 |
| | | AW496808 AI080480 AI631703 AI651023 AI867418 AW818140 AA502500 |
| | | AI206199 AI671282 AI352545 BE501030 AI652535 BE465762 AA206331 |
| | | AW451866 AA471088 AA206342 AA204834 AA206100 AW021661 AA332922 |
| | | N66048 AA703396 H92278 AW139734 H92683 U87589 U87595 H69001 |
| | | U87594 BE466420 AI624817 BE466611 AI206344 AA574397 AA348354 |
| | | AI493192 |
| 118584 | 532052_1 | AW136928 AI685655 BE218584 BE465078 N68963 AA975338 BE147199 |
| | | N76377 |
| 103743 | 112194_1 | AA075998 AA075999 AA070986 AA070896 AA129207 AA078942 AA070783 |
| | | AA078941 |
| 103744 | 114161_1 | AA079267 AA076003 |
| 103746 | 113452_1 | AA075000 AA081876 |
| 103761 | 114208_1 | AA765163 AW298222 AA126126 AA085138 AA076068 |
| 103763 | 48290_6 | AA085291 AA085354 |
| 120209 | 1531817_1 | F02951 Z40892 F04711 |
| 120284 | 158963_1 | AA179656 AA182626 AA182603 |
| 112540 | 1605263_1 | R69751 R70467 H69771 H80879 H80878 |
| 111904 | 1719336_1 | Z41572 R39330 |
| 121059 | 273450_1 | AA393283 AA398628 |
| 121094 | 275729_1 | AA402505 AA398900 |
| 114106 | 1182096_1 | AW602528 BE073859 Z38412 |
| 130091 | 23961_-3 | W88999 |
| 122264 | 296527_1 | AA436837 AA442594 |
| 108280 | 110682_1 | AA065069 AA085108 |
| 129961 | 1706092_1 | R23053 R79884 R76271 |
| 130529 | 158447_1 | AA178953 AA192740 |
| 108309 | 111495_1 | AA069818 AA069971 AA069923 AA069908 |
| 107832 | genbank_AA021473 | AA021473 |
| 123731 | genbank_AA609839 | AA609839 |
| 116571 | genbank_D45652 | D45652 |
| 132225 | genbank_AA128980 | AA128980 |
| 125017 | genbank_T68875 | T68875 |
| 125063 | genbank_T85352 | T85352 |
| 125064 | genbank_T85373 | T85373 |
| 100964 | entrez_J00212 | J00212 |
| 125118 | 149288_1 | R10606 T97620 AA576309 |
| 102269 | entrez_U30245 | U30245 |
| 125150 | NOT_FOUND_entrez_W38240 | W38240 |
| 116801 | genbank_H43879 | H43879 |
| 118111 | genbank_N55493 | N55493 |
| 118129 | genbank_N57493 | N57493 |
| 118329 | genbank_N63520 | N63520 |
| 118475 | genbank_N66845 | N66845 |
| 111490 | genbank_R06862 | R06862 |
| 111514 | genbank_R07998 | R07998 |
| 104534 | R22303_at | R22303 |
| 120340 | genbank_AA206828 | AA206828 |
| 120376 | genbank_AA227469 | AA227469 |
| 104787 | genbank_AA027317 | AA027317 |
| 120409 | genbank_AA235050 | AA235050 |
| 120745 | genbank_AA302809 | AA302809 |
| 120809 | genbank_AA346495 | AA346495 |
| 120839 | genbank_AA348913 | AA348913 |
| 113702 | genbank_T97307 | T97307 |
| 115001 | genbank_AA251376 | AA251376 |
| 122562 | genbank_AA452156 | AA452156 |
| 122635 | genbank_AA454085 | AA454085 |
| 108244 | genbank_AA062839 | AA062839 |
| 108277 | genbank_AA064859 | AA064859 |
| 122723 | genbank_AA457380 | AA457380 |
| 124028 | genbank_F04112 | F04112 |
| 108403 | genbank_AA075374 | AA075374 |
| 122860 | genbank_AA464414 | AA464414 |
| 108427 | genbank_AA076382 | AA076382 |
| 108439 | genbank_AA078986 | AA078986 |
| 131353 | 231290_1 | AW411259 H23555 AW015049 AI684275 AW015886 AW068953 AW014085 |
| | | AI027260 R52686 AA918278 AI129462 AA969360 N34869 AI948416 |
| | | AA534205 AA702483 AA705292 |
| 108533 | genbank_AA084415 | AA084415 |
| 117031 | genbank_H88353 | H88353 |
| 124254 | genbank_H69899 | H69899 |
| 101447 | entrez_M21305 | M21305 |
| 101458 | entrez_M22092 | M22092 |
| 124577 | genbank_N68300 | N68300 |
| 108940 | genbank_AA148603 | AA148603 |
| 108941 | genbank_AA148650 | AA148650 |
| 124627 | genbank_N74625 | N74625 |
| 124720 | 144582_1 | R05283 R11056 |
| 124793 | genbank_R44519 | R44519 |
| 124799 | genbank_R45088 | R45088 |
| 117683 | genbank_N40180 | N40180 |
| 117732 | genbank_N46452 | N46452 |
| 124991 | genbank_T50116 | T50116 |
| 119023 | genbank_N98488 | N98488 |
| 119239 | 95573_2 | T11483 T11472 |
| 119558 | NOT_FOUND_entrez_W38194 | W38194 |
| 119654 | genbank_W57759 | W57759 |
| 105246 | genbank_AA226879 | AA226879 |
| 121350 | genbank_AA405237 | AA405237 |
| 121558 | genbank_AA412497 | AA412497 |
| 105985 | genbank_AA406610 | AA406610 |
| 100071 | entrez_A28102 | A28102 |
| 114648 | genbank_AA101056 | AA101056 |
| 121895 | genbank_AA427396 | AA427396 |
| 100327 | entrez_D55640 | D55640 |
| 123315 | 714071_1 | AA496369 AA496646 |

Table 1A shows the accession numbers for those pkeys lacking unigeneID's for Tables 1. The pkeys in Table 7 lacking unigeneID's are represented within Tables 1-6A For each probeset we have listed the gene cluster number from which the oligonucleotides were designed. Gene clusters were compiled using sequences derived from Genbank ESTs and mRNAs. These sequences were clustered based on sequence similarity using Clustering and Alignment Tools (DoubleTwist, Oakland California). The Genbank accession numbers for sequences comprising each cluster are listed in the “Accession” column.

TABLE 2

| | |
|---|---|
| Pkey: | Unique Eos probeset identifier number |
| Accession: | Accession number used for previous patent filings |
| ExAccn: | Exemplar Accession number, Genbank accession number |
| UnigeneID: | Unigene number |
| Unigene Title: | Unigene gene title |

| Pkey | Accession | ExAccn | UnigeneID | UnigeneTitle |
|---|---|---|---|---|
| 100420 | 100420 | D86983 | Hs.118893 | Melanoma associated gene |
| 100484 | 100484 | NM_005402 | Hs.288757 | v-ral simian leukemia viral oncogene hom |
| 100991 | 100991 | J03836 | Hs.82085 | serine (or cysteine) proteinase inhibito |
| 101168 | 101168 | NM_005308 | Hs.211569 | G protein-coupled receptor kinase 5 |
| 101261 | 101261 | D30857 | Hs.82353 | protein C receptor, endothelial (EPCR) |
| 101447 | 101447 | M21305 | | gb: Human alpha satellite and satellite 3 |
| 101543 | 101543 | M31166 | Hs 2050 | pentaxin-related gene, rapidly induced b |
| 101560 | 101560 | AW958272 | Hs.347326 | intercellular adhesion molecule 2 |
| 101714 | 101714 | M68874 | Hs.211587 | phospholipase A2, group IVA (cytosolic, |
| 101838 | 101838 | BE243845 | Hs.75511 | connective tissue growth factor |
| 102012 | 102012 | BE259035 | Hs 118400 | singed (Drosophila)-like (sea urchin fas |
| 102164 | 102164 | NM_000107 | Hs.77602 | damage-specific DNA binding protein 2 (4 |
| 102283 | 102283 | AW161552 | Hs.83381 | guanine nucleotide binding protein 11 |
| 102564 | 102564 | U59423 | Hs 79067 | MAD (mothers against decapentaplegic, Dr |
| 102759 | 102759 | NM_005100 | Hs.788 | A kinase (PRKA) anchor protein (gravin) |
| 102804 | 102804 | NM_002318 | Hs.83354 | lysyl oxidase-like 2 |
| 102898 | 102898 | NM_002205 | Hs.149609 | integrin, alpha 5 (fibronectin receptor, |
| 103036 | 103036 | M13509 | Hs.83169 | matrix metalloproteinase 1 (interstitial |
| 103095 | 103095 | NM_005424 | Hs.78824 | tyrosme kinase with immunoglobulin and |
| 103166 | 103166 | AA159248 | Hs.180909 | peroxiredoxin 1 |
| 103280 | 103280 | U84722 | Hs.76206 | cadherin 5, type 2, VE-cadherin (vascula |
| 103850 | 103850 | AA187101 | Hs.213194 | hypothetical protein MGC10895 |
| 104592 | 104592 | AW630488 | Hs.25338 | protease, serine, 23 |
| 104786 | 104786 | AA027167 | Hs.10031 | KIAA0955 protein |
| 104865 | 104865 | T79340 | Hs 22575 | B-cell CLL/lymphoma 6, member B (zinc fi |
| 104952 | 104952 | AW076098 | Hs 345588 | desmoplakin (DPI, DPII) |
| 105178 | 105178 | AA313825 | Hs 21941 | AD036 protein |
| 105330 | 105330 | AW338625 | Hs 22120 | ESTs |
| 105729 | 105729 | H46612 | Hs.293815 | Homo sapiens HSPC285 mRNA, partial cds |
| 105977 | 105977 | AK001972 | Hs.30822 | hypothetical protein FLJ11110 |
| 106031 | 106031 | X64116 | Hs.171844 | Homo sapiens cDNA: FLJ22296 fis, clone H |
| 106155 | 106155 | AA425414 | Hs.33287 | nuclear factor I/B |
| 106423 | 106423 | AB020722 | Hs.16714 | Rho guanine exchange factor (GEF) 15 |
| 107174 | 107174 | BE122762 | Hs.25338 | ESTs |
| 107295 | 107295 | AA186629 | Hs 80120 | UDP-N-acetyl-alpha-D-galactosamine:polyp |
| 108756 | 108756 | AA127221 | Hs 117037 | ESTs |
| 108888 | 108888 | AA135606 | Hs.189384 | gb: zl10a05.s1 Soares_pregnant_uterus_NbH |
| 109166 | 109166 | AA219691 | Hs.73625 | RAB6 interacting, kinesin-like (rabkines |
| 109768 | 109768 | F06838 | Hs.14763 | ESTs |
| 110906 | 110906 | AA035211 | Hs.17404 | ESTs |
| 111006 | 111006 | BE387014 | Hs.166146 | Homer, neuronal immediate early gene, 3 |
| 111133 | 111133 | AW580939 | Hs.97199 | complement component C1q receptor |
| 113073 | 113073 | N39342 | Hs.103042 | microtubule-associated protein 1B |
| 113923 | 113923 | AW953484 | Hs.3849 | hypothetical protein FLJ22041 similar to |
| 115061 | 115061 | AI751438 | Hs.41271 | Homo sapiens mRNA full length insert cDN |
| 115145 | 115145 | AA740907 | Hs.88297 | ESTs |
| 115947 | 115947 | R47479 | Hs.94761 | KIAA1691 protein |
| 116339 | 116339 | AK000290 | Hs.44033 | dipeptidyl peptidase 8 |
| 116589 | 116589 | AI557212 | Hs.17132 | ESTs, Moderately similar to I54374 gene |
| 117023 | 117023 | AW070211 | Hs.102415 | Homo sapiens mRNA; cDNA DKFZp586N0121 (f |
| 117563 | 117563 | AF055634 | Hs.44553 | unc5 (C. elegans homolog) c |
| 118475 | 118475 | N66845 | | gb: za46c11.s1 Soares fetal liver spleen |
| 119073 | 119073 | BE245360 | Hs.279477 | ESTs |
| 119174 | 119174 | R71234 | | gb: yi54c08.s1 Soares placenta Nb2HP Homo |
| 119416 | 119416 | T97186 | | gb: ye50h09.s1 Soares fetal liver spleen |
| 121335 | 121335 | AA404418 | | gb: zw37e02.s1 Soares_total_fetus_Nb2HF8_{—} |
| 123160 | 123160 | AA488687 | Hs.284235 | ESTs, Weakly similar to I38022 hypotheti |
| 123523 | 123523 | AA608588 | | gb: ae54e06.s1 Stratagene lung carcinoma |
| 123964 | 123964 | C13961 | | gb: C13961 Clontech human aorta polyA + mR |
| 124315 | 124315 | NM_005402 | Hs.288757 | v-ral simian leukemia viral oncogene hom |
| 124669 | 124669 | AI571594 | Hs.102943 | hypothetical protein MGC12916 |
| 124875 | 124875 | AI887664 | Hs.285814 | sprouty (Drosophila) homolog 4 |
| 125103 | 125103 | AA570056 | Hs.122730 | ESTs, Moderately similar to KIAA1215 pro |
| 125565 | 125565 | R20840 | | gb: yg05c08.r1 Soares infant brain 1NIB H |
| 126511 | 126511 | T92143 | Hs.57958 | EGF-TM7-latrophilin-related protein |
| 126649 | 126649 | AA001860 | Hs.279531 | ESTs |
| 449602 | 449602 | AA001860 | Hs.279531 | ESTs |
| 127402 | 127402 | AA358869 | Hs.227949 | SEC13 (S. cerevisiae)-like 1 |
| 128992 | 128992 | H04150 | Hs.107708 | ESTs |
| 129188 | 129188 | NM_001078 | Hs.109225 | vascular cell adhesion molecule 1 |
| 129371 | 129371 | X06828 | Hs.110802 | von Willebrand factor |
| 129765 | 129765 | M86933 | Hs.1238 | amelogenin (Y chromosome) |
| 129884 | 129884 | AF055581 | Hs 13131 | lysosomal |
| 130639 | 130639 | AI557212 | Hs.17132 | ESTs, Moderately similar to I54374 gene |
| 130828 | 130828 | AW631469 | Hs.203213 | ESTs |
| 131080 | 131080 | NM_001955 | Hs.2271 | endothelin 1 |
| 131182 | 131182 | AI824144 | Hs.23912 | ESTs |
| 131573 | 131573 | AA040311 | Hs.28959 | ESTs |
| 131756 | 131756 | AA443966 | Hs 31595 | ESTs |
| 131881 | 131881 | AW361018 | Hs 3383 | upstream regulatory element binding prot |
| 132083 | 132083 | BE386490 | Hs 279663 | Pirin |
| 132358 | 132358 | NM_003542 | Hs.46423 | H4 histone family, member G |
| 132456 | 132456 | AB011084 | Hs 48924 | KIAA0512 gene product; ALEX2 |
| 132676 | 132676 | N92589 | Hs.261038 | ESTs, Weakly similar to I38022 hypotheti |
| 132718 | 132718 | NM_004600 | Hs.554 | Sjogren syndrome antigen A2 (60 kD, ribon |
| 132760 | 132760 | AA125985 | Hs.56145 | thymosin, beta, identified in neuroblast |
| 132968 | 132968 | AF234532 | Hs 61638 | myosin X |
| 133061 | 133061 | AI186431 | Hs.296638 | prostate differentiation factor |
| 133161 | 133161 | AW021103 | Hs.6631 | hypothetical protein FLJ20373 |
| 133260 | 133260 | AA403045 | Hs.6906 | Homo sapiens cDNA: FLJ23197 fis, clone R |
| 133491 | 133491 | BE619053 | Hs.170001 | eukaryotic translation initiation factor |
| 133550 | 133550 | AI129903 | Hs.74669 | vesicle-associated membrane protein 5 (m |
| 133614 | 133614 | NM_003003 | Hs.75232 | SEC14 (S. cerevisiae)-like 1 |
| 133691 | 133691 | M85289 | Hs.211573 | heparan sulfate proteoglycan 2 (perlecan |
| 133913 | 133913 | AU076964 | Hs.7753 | calumenin |
| 133985 | 133985 | L34657 | Hs 78146 | platelet/endothelial cell adhesion molec |
| 134088 | 134088 | AI379954 | Hs 79025 | KIAA0096 protein |
| 134299 | 134299 | AW580939 | Hs.97199 | complement component C1q receptor |
| 116470 | 116470 | AI272141 | Hs.83484 | SRY (sex determining region Y)-box 4 |
| 134989 | 134989 | AW968058 | Hs.92381 | nudix (nucleoside diphosphate linked moi |
| 135073 | 135073 | W55956 | Hs.94030 | Homo sapiens mRNA; cDNA DKFZp586E162 |
| 100114 | 100114 | X02308 | Hs.82962 | thymidylate synthetase |
| 100143 | 100143 | AU076465 | Hs 278441 | KIAA0015 gene product |
| 100208 | 100208 | NM_002933 | Hs.78224 | ribonuclease, RNase A family, 1 (pancrea |
| 100405 | 100405 | AW291587 | Hs.82733 | nidogen 2 |
| 100455 | 100455 | AW888941 | Hs.75789 | N-myc downstream regulated |
| 100618 | 100618 | AI752163 | Hs.114599 | collagen, type VIII, alpha 1 |
| 100658 | 100658 | U56725 | Hs.180414 | heat shock 70 kD protein 2 |
| 100718 | 100718 | BE295928 | Hs.75424 | inhibitor of DNA binding 1, dominant neg |
| 100828 | 100828 | AL048753 | Hs.303649 | small inducible cytokine A2 (monocyte ch |
| 100991 | 100991 | J03836 | Hs.82085 | serine (or cysteine) proteinase inhibito |
| 101110 | 101110 | AI439011 | Hs.86386 | myeloid cell leukemia sequence 1 (BCL2-r |
| 101156 | 101156 | AA340987 | Hs.75693 | prolylcarboxypeptidase (angiotensinase C |
| 101184 | 101184 | NM_001674 | Hs.460 | activating transcription factor 3 |
| 101317 | 101317 | L42176 | Hs.8302 | four and a half LIM domains 2 |
| 101345 | 101345 | NM_005795 | Hs.152175 | calcitonin receptor-like |
| 101475 | 101475 | BE410405 | Hs 76288 | calpain 2, (m/ll) large subunit |
| 101496 | 101496 | X12784 | Hs.119129 | collagen, type IV, alpha 1 |
| 101543 | 101543 | M31166 | Hs.2050 | pentaxin-related gene, rapidly induced b |
| 101560 | 101560 | AW958272 | Hs.347326 | intercellular adhesion molecule 2 |
| 101592 | 101592 | AF064853 | Hs.91299 | guanine nucleotide binding protein (G pr |
| 101634 | 101634 | AV650262 | Hs.75765 | GRO2 oncogene |
| 101682 | 101682 | AF043045 | Hs.81008 | filamin B, beta (actin-binding protein-2 |
| 101720 | 101720 | M69043 | Hs.81328 | nuclear factor of kappa light polypeptid |
| 101744 | 101744 | AI879352 | Hs.118625 | hexokinase 1 |
| 101837 | 101837 | M92843 | Hs 343586 | zinc finger protein homologous to Zfp-36 |
| 101840 | 101840 | AA236291 | Hs.183583 | serine (or cysteine) proteinase inhibito |
| 101864 | 101864 | BE392588 | Hs 75777 | transgelin |
| 101966 | 101966 | X96438 | Hs.76095 | immediate early response 3 |
| 102013 | 102013 | BE616287 | Hs.178452 | catenin (cadherin-associated protein), a |
| 102059 | 102059 | AI752666 | Hs.76669 | nicotinamide N-methyltransferase |
| 102283 | 102283 | AW161552 | Hs.83381 | guanine nucleotide binding protein 11 |
| 102378 | 102378 | AU076887 | Hs.28491 | spermidine/spermine N1-acetyltransferase |
| 102460 | 102460 | U48959 | Hs.211582 | myosin, light polypeptide kinase |
| 102499 | 102499 | BE243877 | Hs.76941 | ATPase, Na+/K+ transporting, beta 3 poly |
| 102560 | 102560 | R97457 | Hs.63984 | cadherin 13, H-cadherin (heart) |
| 102589 | 102589 | AU076728 | Hs 8867 | cysteine-rich, angiogenic inducer, 61 |
| 102645 | 102645 | AL119566 | Hs 6721 | lysosomal |
| 102693 | 102693 | AA532780 | Hs.183684 | eukaryotic translation initiation factor |
| 102759 | 102759 | NM_005100 | Hs.788 | A kinase (PRKA) anchor protein (gravin) |
| 102882 | 102882 | AI767736 | Hs.290070 | gelsolin (amyloidosis, Finnish type) |
| 102915 | 102915 | X07820 | Hs.2258 | matrix metalloproteinase 10 (stromelysin |
| 102960 | 102960 | AI904738 | Hs.76053 | DEAD/H (Asp-Glu-Ala-Asp/His) box polypep |
| 103020 | 103020 | X53416 | Hs.195464 | filamin A, alpha (actin-binding protein- |
| 103036 | 103036 | M13509 | Hs 83169 | matrix metalloproteinase 1 (interstitial |
| 103080 | 103080 | AU077231 | Hs.82932 | cyclin D1 (PRAD1: parathyroid adenomatos |
| 103138 | 103138 | X65965 | | gb: H. sapiens SOD-2 gene for manganese su |
| 103195 | 103195 | AA351647 | Hs 2642 | eukaryotic translation elongation factor |
| 103371 | 103371 | X91247 | Hs.13046 | thioredoxin reductase 1 |
| 103471 | 103471 | Y00815 | Hs.75216 | protein tyrosine phosphatase, receptor t |
| 104447 | 104447 | AW204145 | Hs.156044 | ESTs |
| 104783 | 104783 | AA533513 | Hs 93659 | protein disulfide isomerase related prot |
| 104865 | 104865 | T79340 | Hs 22575 | B-cell CLL/lymphoma 6, member B (zinc fi |
| 104894 | 104894 | AF065214 | Hs.18858 | phospholipase A2, group IVC (cytosolic, |
| 105113 | 105113 | AB037816 | Hs.8982 | Homo sapiens, clone IMAGE: 3506202, mRNA, |
| 105196 | 105196 | W84893 | Hs 9305 | angiotensin receptor-like 1 |
| 105263 | 105263 | AW388633 | Hs.6682 | solute carrier family 7, (cationic amino |
| 105330 | 105330 | AW338625 | Hs.22120 | ESTs |
| 105492 | 105492 | AI805717 | Hs 289112 | CGI-43 protein |
| 105594 | 105594 | AB024334 | Hs.25001 | tyrosine 3-monooxygenase/tryptophan 5-mo |
| 105732 | 105732 | AW504170 | Hs.274344 | hypothetical protein MGC12942 |
| 105882 | 105882 | W46802 | Hs 81988 | disabled (Drosophila) homolog 2 (mitogen |
| 106031 | 106031 | X64116 | Hs.171844 | Homo sapiens cDNA: FLJ22296 fis, clone H |
| 106222 | 106222 | AA356392 | Hs.21321 | Homo sapiens clone FLB9213 PR02474 mRNA, |
| 106263 | 106263 | W21493 | Hs.28329 | hypothetical protein FLJ14005 |
| 106366 | 106366 | AA186715 | Hs.336429 | RIKENcDNA9130422N19gene |
| 106634 | 106634 | W25491 | Hs 288909 | hypothetical protein FLJ22471 |
| 106793 | 106793 | H94997 | Hs 16450 | ESTs |
| 106842 | 106842 | AF124251 | Hs.26054 | novel SH2-containing protein 3 |
| 106890 | 106890 | AA489245 | Hs.88500 | mitogen-activated protein kinase 8 inter |
| 106974 | 106974 | AI817130 | Hs 9195 | Homo sapiens cDNA FLJ13698 fis, clone PL |
| 107061 | 107061 | BE147611 | Hs.6354 | stromal cell derived factor receptor 1 |
| 107216 | 107216 | D51069 | Hs.211579 | melanoma cell adhesion molecule |
| 107444 | 107444 | W28391 | Hs.343258 | proliferation-associated 2G4, 38 kD |
| 108507 | 108507 | AI554545 | Hs.68301 | ESTs |
| 108931 | 108931 | AA147186 | | gb: zo38d01.s1 Stratagene endothelial cel |
| 109195 | 109195 | AF047033 | Hs.132904 | solute carrier family 4, sodium bicarbon |
| 109456 | 109456 | AW956580 | Hs.42699 | ESTs |
| 110411 | 110411 | AW001579 | Hs.9645 | Homo sapiens mRNA for KIAA1741 protein, |
| 110906 | 110906 | M035211 | Hs.17404 | ESTs |
| 111091 | 111091 | AA300067 | Hs.33032 | hypothetical protein DKFZp434N185 |
| 111378 | 111378 | AW160993 | Hs.326292 | hypothetical gene DKFZp434A1 1 14 |
| 111769 | 111769 | AW629414 | Hs.24230 | ESTs |
| 112951 | 112951 | AA307634 | Hs.6650 | vacuolar protein sorting 45B (yeast homo |
| 113195 | 113195 | H83265 | Hs.8881 | ESTs, Weakly similar to S41044 chromosom |
| 113542 | 113542 | H43374 | Hs 7890 | Homo sapiens mRNA for KIM1671 protein, |
| 113847 | 113847 | NM_005032 | Hs.4114 | plastin 3 (T isoform) |
| 113947 | 113947 | W84768 | | gb: zh53d03.s1 Soares_fetal_liver_spleen_{—} |
| 115061 | 115061 | AI751438 | Hs.41271 | Homo sapiens mRNA full length insert cDN |
| 115870 | 115870 | NM_005985 | Hs.48029 | snail 1 (drosophila homolog), zinc finge |
| 116228 | 116228 | AI767947 | Hs 50841 | ESTs |
| 116314 | 116314 | AI799104 | Hs.178705 | Homo sapiens cDNA FLJ11333 fis, clone PL |
| 117023 | 117023 | AW070211 | Hs.102415 | Homo sapiens mRNA; cDNA DKFZp586N0121 (f |
| 117156 | 117156 | W73853 | | ESTs |
| 117280 | 117280 | M18217 | Hs.172129 | Homo sapiens cDNA: FLJ21409 fis, clone C |
| 119866 | 119866 | AA496205 | Hs.193700 | Homo sapiens mRNA; cDNA DKFZp586l0324 (f |
| 121314 | 121314 | W07343 | Hs 182538 | phospholipid scramblase 4 |
| 121822 | 121822 | AI743860 | | metallothionein 1E (functional) |
| 122331 | 122331 | AL133437 | Hs.110771 | Homo sapiens cDNA: FLJ21904 fis, clone H |
| 123160 | 123160 | AA488687 | Hs 284235 | ESTs, Weakly similar to I38022 hypotheti |
| 124059 | 124059 | BE387335 | Hs 283713 | ESTs, Weakly similar to S64054 hypotheti |
| 124358 | 124358 | AW070211 | Hs.102415 | Homo sapiens mRNA; cDNA DKFZp586N0121 (f |
| 124726 | 124726 | NM_003654 | Hs.104576 | carbohydrate (keratan sulfate Gal-6) sul |
| 125167 | 125167 | AL137540 | Hs 102541 | netrin 4 |
| 125307 | 125307 | AW580945 | Hs.330466 | ESTs |
| 107985 | 107985 | T40064 | Hs.71968 | Homo sapiens mRNA; cDNA DKFZp564F053 (fr |
| 125598 | 125598 | T40064 | Hs.71968 | Homo sapiens mRNA; cDNA DKFZp564F053 (fr |
| 413731 | 413731 | BE243845 | Hs.75511 | connective tissue growth factor |
| 116024 | 116024 | AA088767 | Hs.83883 | transmembrane, prostate androgen induced |
| 418000 | 418000 | M932794 | Hs.83147 | guanine nucleotide binding protein-like |
| 126399 | 126399 | AA088767 | Hs.83883 | transmembrane, prostate androgen induced |
| 127566 | 127566 | AI051390 | Hs.116731 | ESTs |
| 128453 | 128453 | X02761 | Hs 287820 | fibronectin 1 |
| 128515 | 128515 | BE395085 | Hs.10086 | type I transmembrane protein Fn14 |
| 128623 | 128623 | BE076608 | Hs.105509 | CTL2 gene |
| 128669 | 128669 | W28493 | Hs.180414 | heat shock 70 kD protein 8 |
| 128914 | 128914 | AW867491 | Hs.107125 | plasmalemma vesicle associated protein |
| 129188 | 129188 | NM_001078 | Hs.109225 | vascular cell adhesion molecule 1 |
| 129265 | 129265 | AA530892 | Hs.171695 | dual specificity phosphatase 1 |
| 129468 | 129468 | AW410538 | Hs.111779 | secreted protein, acidic, cysteine-rich |
| 101838 | 101838 | BE243845 | Hs.75511 | connective tissue growth factor |
| 129619 | 129619 | AA209534 | Hs.284243 | tetraspan NET-6 protein |
| 129762 | 129762 | AA453694 | Hs.12372 | tripartite motif protein TRIM2 |
| 130018 | 130018 | AA353093 | | metallothionein 1L |
| 130178 | 130178 | U20982 | Hs.1516 | insulin-like growth factor-binding prote |
| 130431 | 130431 | AW505214 | Hs.155560 | calnexin |
| 130553 | 130553 | AF062649 | Hs.252587 | pituitary tumor-transforming 1 |
| 130639 | 130639 | AI557212 | Hs.17132 | ESTs, Moderately similar to I54374 gene |
| 130686 | 130686 | BE548267 | Hs.337986 | Homo sapiens cDNA FLJ10934 fis, clone 0V |
| 130818 | 130818 | AW190920 | Hs.19928 | hypothetical protein SP329 |
| 130899 | 130899 | AI077288 | Hs.296323 | serum/glucocorticoid regulated kinase |
| 131080 | 131080 | NM_001955 | Hs.2271 | endothelin 1 |
| 131091 | 131091 | AJ271216 | Hs.22880 | dipeptidylpeptidase III |
| 131182 | 131182 | AI824144 | Hs 23912 | ESTs |
| 131319 | 131319 | NM_003155 | Hs 25590 | stanniocalcin 1 |
| 131328 | 131328 | AW939251 | Hs.25647 | v-fos FBJ murine osteosarcoma viral onco |
| 131328 | 131328 | AW939251 | Hs.25647 | v-fos FBJ murine osteosarcoma viral onco |
| 131555 | 131555 | T47364 | Hs 278613 | interferon, alpha-inducible protein 27 |
| 131573 | 131573 | AA040311 | Hs.28959 | ESTs |
| 131756 | 131756 | AA443966 | Hs 31595 | ESTs |
| 131909 | 131909 | NM_016558 | Hs.274411 | SCAN domain-containing 1 |
| 132046 | 132046 | AI359214 | Hs.179260 | chromosome 14 open reading frame 4 |
| 132151 | 132151 | BE379499 | Hs.173705 | Homo sapiens cDNA: FLJ22050 fis, clone H |
| 132187 | 132187 | AA235709 | Hs 4193 | DKFZP58601624 protein |
| 132314 | 132314 | AF112222 | Hs.323806 | pinin, desmosome associated protein |
| 132398 | 132398 | AA876616 | Hs.16979 | ESTs, Weakly similar to A43932 mucin 2 p |
| 132490 | 132490 | NM_001290 | Hs.4980 | LIM domain binding 2 |
| 132546 | 132546 | M24283 | Hs 168383 | intercellular adhesion molecule 1 (CD54) |
| 132716 | 132716 | BE379595 | Hs 283738 | casein kinase 1 , alpha 1 |
| 132883 | 132883 | AA373314 | Hs 5897 | Homo sapiens mRNA; cDNA DKFZp586P1622 (f |
| 132989 | 132989 | AA480074 | Hs.331328 | hypothetical protein FLJ13213 |
| 133071 | 133071 | BE384932 | Hs.64313 | ESTs, Weakly similar to AF257182 1 G-pro |
| 133099 | 133099 | W16518 | Hs.279518 | amyloid beta (A4) precursor-like protein |
| 133149 | 133149 | AA370045 | Hs 6607 | AXIN1 up-regulated |
| 133200 | 133200 | AB037715 | Hs 183639 | hypothetical protein FLJ10210 |
| 133260 | 133260 | AA403045 | Hs 6906 | Homo sapiens cDNA FLJ23197 fis, clone R |
| 133349 | 133349 | AW631255 | Hs.8110 | L-3-hydroxyacyl-Coenzyme A dehydrogenase |
| 133398 | 133398 | NM_000499 | Hs.72912 | cytochrome P450, subfamily I (aromatic c |
| 133454 | 133454 | BE547647 | Hs.177781 | hypothetical protein MGC5618 |
| 133491 | 133491 | BE619053 | Hs.170001 | eukaryotic translation initiation factor |
| 133517 | 133517 | NM_000165 | Hs.74471 | gap junction protein, alpha 1, 43 kD (con |
| 133538 | 133538 | NM_003257 | Hs.74614 | tight junction protein 1 (zona occludens |
| 133584 | 133584 | D90209 | Hs.181243 | activating transcription factor 4 (tax-r |
| 133617 | 133617 | BE244334 | Hs 75249 | ADP-ribosylation factor-like 6 interacti |
| 133671 | 133671 | AW503116 | Hs.301819 | zinc finger protein 146 |
| 133681 | 133681 | AI352558 | | tyrosine 3-monooxygenase/tryptophan 5-mo |
| 133730 | 133730 | BE242779 | Hs.179526 | upregulated by 1,25-dihydroxyvitamm D-3 |
| 133802 | 133802 | AW239400 | Hs 76297 | G protein-coupled receptor kinase 6 |
| 133838 | 133838 | BE222494 | Hs.180919 | inhibitor of DNA binding 2, dominant neg |
| 133889 | 133889 | U48959 | Hs 211582 | myosin, light polypeptide kinase |
| 133975 | 133975 | C18356 | Hs.295944 | tissue factor pathway inhibitor 2 |
| 134039 | 134039 | NM_002290 | Hs.78672 | laminin, alpha 4 |
| 134081 | 134081 | AL034349 | Hs.79005 | protein tyrosine phosphatase, receptor t |
| 134203 | 134203 | AA161219 | Hs.799 | diphtheria toxin receptor (heparin-bindi |
| 134299 | 134299 | AW580939 | Hs 97199 | complement component C1q receptor |
| 134339 | 134339 | R70429 | Hs.81988 | disabled (Drosophila) homolog 2 (mitogen |
| 134381 | 134381 | AI557280 | Hs.184270 | capping protein (actin filament) muscle |
| 134416 | 134416 | X68264 | Hs.211579 | melanoma cell adhesion molecule |
| 134558 | 134558 | NM_001773 | Hs 85289 | CD34 antigen |
| 134983 | 134983 | D28235 | Hs.196384 | prostaglandin-endoperoxide synthase 2 (p |
| 135052 | 135052 | AL136653 | Hs.93675 | decidual protein induced by progesterone |
| 135069 | 135069 | AA876372 | Hs.93961 | Homo sapiens mRNA; cDNA DKFZp667D095 (fr |
| 135073 | 135073 | W55956 | Hs 94030 | Homo sapiens mRNA; cDNA DKFZp586E1624 (f |
| 135196 | 135196 | C03577 | Hs.9615 | myosin regulatory light chain 2, smooth |
| 134404 | 134404 | AB000450 | Hs 82771 | vaccinia related kinase 2 |
| 100082 | 100082 | AA130080 | Hs 4295 | proteasome (prosome, macropain) 26S subu |
| 130150 | 130150 | BE094848 | Hs.15113 | homogentisate 1,2-dioxygenase (homogenti |
| 130839 | 130839 | AB011169 | Hs 20141 | similar to S. cerevisiae SSM4 |
| 100113 | 100113 | NM_001269 | Hs.84746 | chromosome condensation 1 |
| 100129 | 100129 | AA469369 | Hs.5831 | tissue inhibitor of metalloproteinase 1 |
| 100169 | 100169 | AL037228 | Hs.82043 | D123 gene product |
| 100190 | 100190 | M91401 | Hs.178658 | RAD23 (S. cerevisiae) homolog B |
| 100211 | 100211 | D26528 | Hs 123058 | DEAD/H (Asp-Glu-Ala-Asp/His) box polypep |
| 130283 | 130283 | NM_012288 | Hs.153954 | TRAM-like protein |
| 100248 | 100248 | NM_015156 | Hs 78398 | KIAA0071 protein |
| 100262 | 100262 | D38500 | Hs.278468 | postmeiotic segregation increased 2-like |
| 100281 | 100281 | AF091035 | Hs.184627 | KIAA0118 protein |
| 100327 | 100327 | D55640 | | gb: Human monocyte PABL (pseudoautosomal |
| 134495 | 134495 | D63477 | Hs 84087 | KIAA0143 protein |
| 135152 | 135152 | M96954 | Hs.182741 | TIA1 cytotoxic granule-associated RNA-bi |
| 100372 | 100372 | NM_014791 | Hs.184339 | KIAA01 75 gene product |
| 100394 | 100394 | D84284 | Hs.66052 | CD38 antigen (p45) |
| 100418 | 100418 | D86978 | Hs.84790 | KIAA0225 protein |
| 134347 | 134347 | AF164142 | Hs.82042 | solute earner family 23 (nucleobase tra |
| 100438 | 100438 | AA013051 | Hs 91417 | topoisomerase (DNA) II binding protein |
| 100481 | 100481 | X70377 | Hs.121489 | cystatin D |
| 100591 | 100591 | NM_004091 | Hs.231444 | Homo sapiens, Similar to hypothetical pr |
| 100662 | 100662 | AI368680 | Hs.816 | SRY (sex determining region Y)-box 2 |
| 100905 | 100905 | L12260 | Hs.172816 | neuregulin 1 |
| 100950 | 100950 | AF128542 | Hs 166846 | polymerase (DNA directed), epsilon |
| 135407 | 135407 | J04029 | Hs 99936 | keratin 10 (epidermolytic hyperkeratosis |
| 131877 | 131877 | J04088 | Hs.156346 | topoisomerase (DNA) II alpha (170 kD) |
| 134786 | 134786 | T29618 | Hs 89640 | TEK tyrosme kinase, endothelial (venous |
| 134078 | 134078 | L08895 | Hs.78995 | MADS box transcription enhancer factor 2 |
| 134849 | 134849 | BE409525 | Hs.902 | neurofibromin 2 (bilateral acoustic neur |
| 101152 | 101152 | AI984625 | Hs.9884 | spindle pole body protein |
| 131687 | 131687 | BE297635 | Hs.3069 | heat shock 70 kD protein 9B (mortalin-2) |
| 421155 | 421155 | H87879 | Hs.102267 | lysyl oxidase |
| 133975 | 133975 | C18356 | Hs.295944 | tissue factor pathway inhibitor 2 |
| 130155 | 130155 | AA101043 | Hs.151254 | kallikrein 7 (chymotryptic, stratum corn |
| 132813 | 132813 | BE313625 | Hs.57435 | solute carrier family 11 (proton-coupled |
| 101300 | 101300 | BE535511 | | transmembrane trafficking protein |
| 130344 | 130344 | AW250122 | Hs.154879 | DiGeorge syndrome critical region gene D |
| 101381 | 101381 | AW675039 | Hs.1227 | aminolevulinate, delta-, dehydratase |
| 133780 | 133780 | AA557660 | Hs.76152 | decorin |
| 101447 | 101447 | M21305 | | gb: Human alpha satellite and satellite 3 |
| 101470 | 101470 | NM_000546 | Hs.1846 | tumor protein p53 (Li-Fraumeni syndrome) |
| 101478 | 101478 | NM_002890 | Hs 758 | RAS p21 protein activator (GTPase activa |
| 133519 | 133519 | AW583062 | Hs.74502 | chymotrypsinogen B1 |
| 134116 | 134116 | R84694 | Hs.79194 | cAMP responsive element binding protein |
| 130174 | 130174 | M29551 | Hs.151531 | protein phosphatase 3 (formerly 2B), cat |
| 132983 | 132983 | M30269 | | nidogen (enactin) |
| 101543 | 101543 | M31166 | Hs.2050 | pentaxin-related gene, rapidly induced b |
| 101620 | 101620 | S55271 | Hs.247930 | Epsilon, IgE |
| 133595 | 133595 | AA393273 | Hs.75133 | transcription factor 6-like 1 (mitochond |
| 101700 | 101700 | D90337 | Hs 247916 | natriuretic peptide precursor C |
| 134246 | 134246 | D28459 | Hs.80612 | ubiquitin-conjugating enzyme E2A (RAD6 h |
| 133948 | 133948 | X59960 | Hs.77813 | sphingomyelin phosphodiesterase 1, acid |
| 133948 | 133948 | X59960 | Hs.77813 | sphingomyelin phosphodiesterase 1, acid |
| 133948 | 133948 | X59960 | Hs.77813 | sphingomyelin phosphodiesterase 1, acid |
| 101812 | 101812 | BE439894 | Hs.78991 | DNA segment, numerous copies, expressed |
| 133396 | 133396 | M96326 | Hs 72885 | azurocidin 1 (cationic antimicrobial pro |
| 129026 | 129026 | AL120297 | Hs.108043 | Friend leukemia virus integration 1 |
| 134831 | 134831 | AA853479 | Hs.89890 | pyruvate carboxylase |
| 134395 | 134395 | AA456539 | Hs.8262 | lysosomal |
| 101977 | 101977 | AF112213 | Hs.184062 | putative Rab5-interacting protein |
| 101998 | 101998 | U01212 | Hs.248153 | olfactory marker protein |
| 102007 | 102007 | U02556 | Hs.75307 | t-complex-associated-testis-expressed 1- |
| 416658 | 416658 | U03272 | Hs 79432 | fibrillin 2 (congenital contractural ara |
| 135389 | 135389 | U05237 | Hs 99872 | fetal Alzheimer antigen |
| 130145 | 130145 | U34820 | Hs.151051 | mitogen-activated protein kinase 10 |
| 420269 | 420269 | U72937 | Hs.96264 | alpha thalassemia/mental retardation syn |
| 102123 | 102123 | NM_001809 | Hs.1594 | centromere protein A (17 kD) |
| 102133 | 102133 | AU076845 | Hs.155596 | BCL2/adenovirus E1B 19 kD-interacting pro |
| 102162 | 102162 | AA450274 | Hs.1592 | CDC16 (cell division cycle 16, S. cerevi |
| 427653 | 427653 | AA159001 | Hs.180069 | nuclear respiratory factor 1 |
| 102200 | 102200 | AA232362 | Hs.157205 | branched chain aminotransferase 1, cytos |
| 102214 | 102214 | U23752 | Hs.32964 | SRY (sex determining region Y)-box 11 |
| 131319 | 131319 | NM_003155 | Hs 25590 | stanniocalcin 1 |
| 132316 | 132316 | U28831 | Hs.44566 | KIAA1641 protein |
| 134365 | 134365 | AA568906 | Hs.82240 | syntaxin 3A |
| 102298 | 102298 | AA382169 | Hs.54483 | N-myc (and STAT) interactor |
| 302344 | 302344 | BE303044 | Hs.192023 | eukaryotic translation initiation factor |
| 102367 | 102367 | U39656 | Hs.118825 | mitogen-activated protein kinase kinase |
| 102394 | 102394 | NM_003816 | Hs.2442 | a disintegrin and metalloproteinase doma |
| 129521 | 129521 | AF071076 | Hs.112255 | nucleoporin 98 kD |
| 102251 | 102251 | NM_004398 | Hs.41706 | DEAD/H (Asp-Glu-Ala-Asp/His) box polypep |
| 133746 | 133746 | AW410035 | Hs.75862 | MAD (mothers against decapentaplegic, Dr |
| 132828 | 132828 | AB014615 | Hs.57710 | fibroblast growth factor 8 (androgen-ind |
| 132828 | 132828 | AB014615 | Hs.57710 | fibroblast growth factor 8 (androgen-ind |
| 130441 | 130441 | U63630 | Hs.155637 | protein kinase, DNA-activated, catalytic |
| 129350 | 129350 | U50535 | Hs.110630 | Human BRCA2 region, mRNA sequence CG006 |
| 130457 | 130457 | AB014595 | Hs.155976 | cullin 4B |
| 102560 | 102560 | R97457 | Hs.63984 | cadherin 13, H-cadherin (heart) |
| 134305 | 134305 | U61397 | Hs.81424 | ubiquitin-like 1 (sentrin) |
| 132736 | 132736 | AW081883 | Hs.211578 | Homo sapiens cDNA: FLJ23037 fis, clone L |
| 102663 | 102663 | NM_002270 | Hs.168075 | karyopherin (importin) beta 2 |
| 102735 | 102735 | AF111106 | Hs.3382 | protein phosphatase 4, regulatory subuni |
| 101175 | 101175 | U82671 | Hs 36980 | melanoma antigen, family A, 2 |
| 132164 | 132164 | AI752235 | Hs 41270 | procollagen-lysine, 2-oxoglutarate 5-dio |
| 102826 | 102826 | NM_007274 | Hs.8679 | cytosolic acyl coenzyme A thioester hydr |
| 102846 | 102846 | BE264974 | Hs.6566 | thyroid hormone receptor interactor 13 |
| 134161 | 134161 | AA634543 | Hs.79440 | IGF-II mRNA-binding protein 3 |
| 302363 | 302363 | AW163799 | Hs.198365 | 2,3-bisphosphoglycerate mutase |
| 125701 | 125701 | T72104 | Hs.93194 | apolipoprotein A-I |
| 134656 | 134656 | AI750878 | Hs.87409 | thrombospondin 1 |
| 102968 | 102968 | AU076611 | Hs.154672 | methylene tetrahydrofolate dehydrogenase |
| 134037 | 134037 | AI808780 | Hs.227730 | integrin, alpha 6 |
| 103023 | 103023 | AW500470 | Hs.117950 | multifunctional polypeptide similar to S |
| 130282 | 130282 | BE245380 | Hs.153952 | 5′ nucleotidase (CD73) |
| 128568 | 128568 | H12912 | Hs 274691 | adenylate kinase 3 |
| 103093 | 103093 | S79876 | Hs 44926 | dipeptidylpeptidase IV (CD26, adenosine |
| 129063 | 129063 | X63094 | Hs.283822 | Rhesus blood group, D antigen |
| 133227 | 133227 | AW977263 | Hs.68257 | general transaction factor IIF, polype |
| 103184 | 103184 | U43143 | Hs.74049 | fms-related tyrosine kinase 4 |
| 103208 | 103208 | AW411340 | Hs.31314 | retinoblastoma-binding protein 7 |
| 131486 | 131486 | F06972 | Hs.27372 | BMX non-receptor tyrosine kinase |
| 103334 | 103334 | NM_001260 | Hs 25283 | cyclin-dependent kinase 8 |
| 135094 | 135094 | NM_003304 | Hs.250687 | transient receptor potential channel 1 |
| 103352 | 103352 | H09366 | Hs.78853 | uracil-DNA glycosylase |
| 132173 | 132173 | X89426 | Hs.41716 | endothelial cell-specific molecule 1 |
| 131584 | 131584 | AA598509 | Hs.29117 | purine-rich element binding protein A |
| 103378 | 103378 | AL119690 | Hs 153618 | HCGVIII-1 protein |
| 103410 | 103410 | AA158294 | Hs.295362 | DR 1-associated protein 1 (negative cofac |
| 103438 | 103438 | AW175781 | Hs.152720 | M-phase phosphoprotein 6 |
| 103452 | 103452 | NM_006936 | Hs 85119 | SMT3 (suppressor of mif two 3, yeast) ho |
| 135185 | 135185 | AW404908 | Hs.96038 | Ric (Drosophila)-like, expressed in many |
| 134662 | 134662 | NM_007048 | Hs.284283 | butyrophilin, subfamily 3, member A1 |
| 103500 | 103500 | AW408009 | Hs.22580 | alkylglycerone phosphate synthase |
| 132084 | 132084 | NM_002267 | Hs 3886 | karyopherin alpha 3 (importin alpha 4) |
| 133152 | 133152 | Z11695 | Hs.324473 | mitogen-activated protein kinase 1 |
| 103612 | 103612 | BE336654 | Hs.70937 | H3 histone family, member A |
| 103692 | 103692 | AW137912 | Hs.227583 | Homo sapiens chromosome X map Xp11 23 L- |
| 129796 | 129796 | BE218319 | Hs.5807 | GTPase Rab14 |
| 132683 | 132683 | BE264633 | Hs.143638 | WD repeat domain 4 |
| 103723 | 103723 | BE274312 | Hs.214783 | Homo sapiens cDNA FLJ14041 fis, clone HE |
| 133260 | 133260 | AA403045 | Hs.6906 | Homo sapiens cDNA: FLJ23197 fis, clone R |
| 103766 | 103766 | AI920783 | Hs 191435 | ESTs |
| 132051 | 132051 | AA393968 | Hs.180145 | HSPC030 protein |
| 135289 | 135289 | AW372569 | Hs.9788 | hypothetical protein MGC10924 similar to |
| 103794 | 103794 | AF244135 | Hs.30670 | hepatocellular carcinoma-associated anti |
| 134319 | 134319 | BE304999 | Hs.285754 | fumarate hydratase |
| 119159 | 119159 | AF142419 | Hs.15020 | homolog of mouse quaking QKI (KH domain |
| 103850 | 103850 | AA187101 | Hs.213194 | hypothetical protein MGC10895 |
| 322026 | 322026 | AW024973 | Hs 283675 | NPD009 protein |
| 103861 | 103861 | AA206236 | Hs.4944 | hypothetical protein FLJ12783 |
| 447735 | 447735 | AA775268 | Hs.6127 | Homo sapiens cDNA. FLJ23020 fis, clone L |
| 131236 | 131236 | AF043117 | Hs 24594 | ubiquitination factor E4B (homologous to |
| 129013 | 129013 | AA371156 | Hs 107942 | DKFZP564M112 protein |
| 103988 | 103988 | AA314389 | Hs.342849 | ADP-ribosylation factor-like 5 |
| 425284 | 425284 | AF155568 | Hs.348043 | NS1-associated protein 1 |
| 133281 | 133281 | AK001601 | Hs.69594 | high-mobility group 20A |
| 108154 | 108154 | NM_005754 | Hs.220689 | Ras-GTPase-activating protein SH3-domain |
| 135073 | 135073 | W55956 | Hs.94030 | Homo sapiens mRNA; cDNA DKFZp586E1624 (f |
| 129593 | 129593 | AI338247 | Hs.98314 | Homo sapiens mRNA; cDNA DKFZp586L0120 (f |
| 132064 | 132064 | AA121098 | Hs.3838 | serum-inducible kinase |
| 131427 | 131427 | AF151879 | Hs.26706 | CGI-121 protein |
| 104282 | 104282 | C14448 | Hs.332338 | EST |
| 130443 | 130443 | D25216 | Hs.155650 | KIAA0014 gene product |
| 132837 | 132837 | AA370362 | Hs.57958 | EGF-TM7-latrophilin-related protein |
| 104334 | 104334 | D82614 | Hs.78771 | phosphoglycerate kinase 1 |
| 134731 | 134731 | D89377 | Hs.89404 | msh (Drosophila) homeo box homolog 2 |
| 131670 | 131670 | H03514 | Hs.15589 | ESTs |
| 104402 | 104402 | H56731 | Hs.132956 | ESTs |
| 129077 | 129077 | N74724 | Hs.108479 | ESTs |
| 134927 | 134927 | L36531 | Hs.91296 | integrin, alpha 8 |
| 134498 | 134498 | AW246273 | Hs 84131 | threonyl-tRNA synthetase |
| 104488 | 104488 | N56191 | Hs.106511 | protocadherin 17 |
| 129214 | 129214 | AL044335 | Hs.109526 | zinc finger protein 198 |
| 104530 | 104530 | AK001676 | Hs.12457 | hypothetical protein FLJ10814 |
| 104544 | 104544 | AI091173 | Hs.222362 | ESTs, Weakly similar to p40 [H. sapiens] |
| 104567 | 104567 | AA040620 | Hs.5672 | hypothetical protein AF140225 |
| 129575 | 129575 | F08282 | Hs 278428 | progestin induced protein |
| 104599 | 104599 | AW815036 | Hs.151251 | ESTs |
| 104667 | 104667 | AI239923 | Hs 63931 | ESTs |
| 104764 | 104764 | AI039243 | Hs.278585 | ESTs |
| 104787 | 104787 | AA027317 | | gb: ze97d11.s1 Soares_fetal_heart_NbHH19W |
| 104804 | 104804 | AI858702 | Hs.31803 | ESTs, Weakly similar to N-WASP [H sapien |
| 130828 | 130828 | AW631469 | Hs.203213 | ESTs |
| 104943 | 104943 | AF072873 | Hs.114218 | frizzled (Drosophila) homolog 6 |
| 105024 | 105024 | AA126311 | Hs.9879 | ESTs |
| 105038 | 105038 | AW503733 | Hs.9414 | KIAA1488 protein |
| 105096 | 105096 | AL042506 | Hs.21599 | Kruppel-like factor 7 (ubiquitous) |
| 105169 | 105169 | BE245294 | Hs 180789 | S164 protein |
| 130401 | 130401 | BE396283 | Hs.173987 | eukaryotic translation initiation factor |
| 130114 | 130114 | AA233393 | Hs.14992 | hypothetical protein FLJ11151 |
| 105337 | 105337 | AI468789 | Hs.347187 | myotubularin related protein 1 |
| 105376 | 105376 | AW994032 | Hs.8768 | hypothetical protein FLJ10849 |
| 131962 | 131962 | AK000046 | Hs.343877 | hypothetical protein FLJ20039 |
| 128658 | 128658 | BE397354 | Hs 324830 | dipthena toxin resistance protein requi |
| 105508 | 105508 | AA173942 | Hs.326416 | Homo sapiens mRNA, cDNA DKFZp564H1916 (f |
| 135172 | 135172 | AB028956 | Hs.12144 | KIAA1033 protein |
| 132542 | 132542 | AL137751 | Hs 263671 | Homo sapiens mRNA; cDNA DKFZp43410812 (f |
| 105659 | 105659 | AA283044 | Hs.25625 | hypothetical protein FLJ11323 |
| 105674 | 105674 | AI609530 | Hs 279789 | histone deacetylase 3 |
| 105722 | 105722 | AI922821 | Hs.32433 | ESTs |
| 115951 | 115951 | BE546245 | Hs.301048 | sec13-like protein |
| 105985 | 105985 | AA406610 | | gb: zv15b10.s1 Soares_NhHMPu_S1 Homo sapi |
| 131216 | 131216 | AI815486 | Hs.243901 | Homo sapiens cDNA FLJ20738 fis, clone HE |
| 113689 | 113689 | AB037850 | Hs.16621 | DKFZP434I116 protein |
| 130839 | 130839 | AB011169 | Hs 20141 | similar to S cerevisiae SSM4 |
| 130777 | 130777 | AW135049 | Hs.26285 | Homo sapiens cDNA FLJ10643 fis, clone NT |
| 106196 | 106196 | AA525993 | Hs.173699 | ESTs, Weakly similar to ALU1_HUMAN ALU S |
| 133200 | 133200 | AB037715 | Hs 183639 | hypothetical protein FLJ10210 |
| 106328 | 106328 | AL079559 | Hs.28020 | KIAA0766 gene product |
| 106423 | 106423 | AB020722 | Hs 16714 | Rho guanine exchange factor (GEF) 15 |
| 439608 | 439608 | AW864696 | Hs.301732 | hypothetical protein MGC5306 |
| 106503 | 106503 | AB033042 | Hs 29679 | cofactor required for Sp1 transcriptiona |
| 106543 | 106543 | AA676939 | Hs.69285 | neuropilin 1 |
| 106589 | 106589 | AK000933 | Hs 28661 | Homo sapiens cDNA FLJ10071 fis, clone HE |
| 106596 | 106596 | AA452379 | | ESTs, Moderately similar to ALU7_HUMAN A |
| 106636 | 106636 | AW958037 | Hs 286 | ribosomal protein L4 |
| 131353 | 131353 | AW754182 | | gb: RC2-CT0321-131199-011-c01 CT0321 Homo |
| 131710 | 131710 | NM_015368 | Hs 30985 | pannexin 1 |
| 131775 | 131775 | AB014548 | Hs 31921 | KIAA0648 protein |
| 106773 | 106773 | AA478109 | Hs.188833 | ESTs |
| 106817 | 106817 | D61216 | Hs.18672 | ESTs |
| 106848 | 106848 | AA449014 | Hs.121025 | chromosome 11 open reading frame 5 |
| 418699 | 418699 | BE539639 | Hs.173030 | ESTs, Weakly similar to ALU8_HUMAN ALU S |
| 130638 | 130638 | AW021276 | Hs.17121 | ESTs |
| 107059 | 107059 | BE614410 | Hs 23044 | RAD51 (S. cerevisiae) homolog (E coli Re |
| 107115 | 107115 | BE379623 | Hs 27693 | peptidylprolyl isomerase (cyclophilin)-l |
| 107156 | 107156 | AA137043 | Hs.9663 | programmed cell death 6-interacting prot |
| 130621 | 130621 | AW513087 | Hs.16803 | LUC7 (S. cerevisiae)-like |
| 132626 | 132626 | AW504732 | Hs 21275 | hypothetical protein FLJ11011 |
| 131610 | 131610 | AA357879 | Hs.29423 | scavenger receptor with C-type lectin |
| 107295 | 107295 | AA186629 | Hs.80120 | UDP-N-acetyl-alpha-D-galactosamine polyp |
| 107315 | 107315 | AA316241 | Hs.90691 | nucleophosmin/nucleoplasmin 3 |
| 107328 | 107328 | AW959891 | Hs 76591 | KIAA0887 protein |
| 134715 | 134715 | U48263 | Hs.89040 | prepronociceptin |
| 129938 | 129938 | AW003668 | Hs.135587 | Human clone 23629 mRNA sequence |
| 130074 | 130074 | AL038596 | Hs.250745 | polymerase (RNA) III (DMA directed) (62k |
| 132036 | 132036 | AL157433 | Hs 37706 | hypothetical protein DKFZp434E2220 |
| 113857 | 113857 | AW243158 | Hs.5297 | DKFZP564A2416 protein |
| 130419 | 130419 | AF037448 | Hs.155489 | NS1-associated protein 1 |
| 132616 | 132616 | BE262677 | Hs.283558 | hypothetical protein PRO1855 |
| 132358 | 132358 | NM_003542 | Hs 46423 | H4 histone family, member G |
| 125827 | 125827 | NM_003403 | Hs 97496 | YY1 transcription factor |
| 107609 | 107609 | R75654 | Hs.164797 | hypothetical protein FLJ13693 |
| 107714 | 107714 | AA015761 | Hs.60642 | ESTs |
| 107832 | 107832 | AA021473 | | gb: ze66c11.s1 Soares retina N2b4HR Homo |
| 124337 | 124337 | N23541 | Hs.281561 | Homo sapiens cDNA: FLJ23582 fis, clone L |
| 129577 | 129577 | N75346 | Hs.306121 | CDC20 (cell division cycle 20, S. cerevi |
| 132000 | 132000 | AW247017 | Hs 36978 | melanoma antigen, family A, 3 |
| 107935 | 107935 | AA029428 | Hs.61555 | ESTs |
| 131461 | 131461 | AA992841 | Hs.27263 | KIAA1458 protein |
| 108029 | 108029 | AA040740 | Hs.62007 | ESTs |
| 108084 | 108084 | AA058944 | Hs.116602 | Homo sapiens, clone IMAGE:4154008, mRNA, |
| 108168 | 108168 | AI453137 | Hs.63176 | ESTs |
| 108189 | 108189 | AW376061 | Hs.63335 | ESTs, Moderately similar to A46010 X-lin |
| 108203 | 108203 | AW847814 | Hs.289005 | Homo sapiens cDNA: FLJ21532 fis, clone C |
| 108217 | 108217 | AA058686 | Hs.62588 | ESTs |
| 108277 | 108277 | AA064859 | | gb: zm50f03.s1 Stratagene fibroblast (937 |
| 108309 | 108309 | AA069818 | | gb: zm67e03.r1 Stratagene neuroepithelium |
| 108340 | 108340 | AA069820 | Hs.180909 | peroxiredoxin 1 |
| 108427 | 108427 | AA076382 | | gb: zm91g08.s1 Stratagene ovarian cancer |
| 108439 | 108439 | AA078986 | | gb: zm92h01.s1 Stratagene ovarian cancer |
| 108469 | 108469 | AA079487 | | gb: zm97f08.s1 Stratagene colon HT29 (937 |
| 108501 | 108501 | AA083256 | | gb: zn08g12 s1 Stratagene hNT neuron (937 |
| 108562 | 108562 | AA100796 | | gb: zm26c06 s1 Stratagene pancreas (93720 |
| 130890 | 130890 | AI907537 | Hs 76698 | stress-associated endoplasmic reticulum |
| 130385 | 130385 | AW067800 | Hs 155223 | stanniocalcin 2 |
| 108807 | 108807 | AI652236 | Hs 49376 | hypothetical protein FLJ20644 |
| 108833 | 108833 | AF188527 | Hs.61661 | ESTs, Weakly similar to AF174605 1 F-box |
| 108846 | 108846 | AL117452 | Hs 44155 | DKFZP586G1517 protein |
| 131474 | 131474 | L46353 | Hs.2726 | high-mobility group (nonhistone chromoso |
| 108941 | 108941 | AA148650 | | gb: zo09e06.s1 Stratagene neuroepithelium |
| 108996 | 108996 | AW995610 | Hs.332436 | EST |
| 131183 | 131183 | AI611807 | Hs.285107 | hypothetical protein FLJ13397 |
| 109022 | 109022 | AA157291 | Hs.21479 | ubinuclein 1 |
| 109068 | 109068 | AA164293 | Hs 72545 | ESTs |
| 129021 | 129021 | AL044675 | Hs.173081 | KIAA0530 protein |
| 109146 | 109146 | AA176589 | Hs.142078 | EST |
| 131080 | 131080 | NM_001955 | Hs.2271 | endothelin 1 |
| 109222 | 109222 | AA192833 | Hs 333512 | similar to rat myomegalin |
| 109481 | 109481 | AA878923 | Hs.289069 | hypothetical protein FLJ21016 |
| 109516 | 109516 | AI471639 | Hs.71913 | ESTs |
| 109556 | 109556 | AI925294 | Hs.87385 | ESTs |
| 109578 | 109578 | F02208 | Hs 27214 | ESTs |
| 109625 | 109625 | H29490 | Hs.22697 | ESTs |
| 109648 | 109648 | H17800 | Hs.7154 | ESTs |
| 109699 | 109699 | H18013 | Hs.167483 | ESTs |
| 109933 | 109933 | R52417 | Hs 20945 | Homo sapiens clone 24993 mRNA sequence |
| 110039 | 110039 | H11938 | Hs 21907 | histone acetyltransferase |

TABLE 2A

| | |
|---|---|
| Pkey: | Unique Eos probeset identifier number |
| CAT number: | Gene cluster number |
| Accession: | Genbank accession numbers |

| Pkey | CAT Number | Accession |
|---|---|---|
| 108469 | 116761_1 | AA079487 AA128547 AA128291 AA079587 AA079600 |
| 108501 | 13684_-12 | AA083256 |
| 108562 | 36375_1 | AA100796 AF020589 AA074629 AA075946 AA100849 AA085347 AA126309 AA079311 |
| | | AA079323 AA085274 |
| 101300 | 4669_1 | BE535511 M62098 AA306787 AW891766 AA348998 AA338869 AA344013 AW956561 |
| | | AW389343 AW403607 L40391 AW408435 AA121738 AI568978 H13317 R20373 |
| | | AW948724 AW948744 AA335023 AA436722 AA448690 C21404 AW884390 |
| | | AA345454 AA303292 AA174174 BE092290 T90614 AA035104 R76028 AA126924 |
| | | AA741086 AW022056 AW118940 AA121666 AI832409 AA683475 AI140901 |
| | | AI623576 AW519064 AW474125 AI953923 AI735349 AW150109 AI436154 |
| | | AW118130 AW270782 AI804073 N27434 AA876543 AA937815 AI051166 |
| | | AA505378 AI041975 AI335355 AI089540 AA662243 AI127912 AI925604 |
| | | AI250880 AI366874 AI564386 AI815196 AI683526 AI435885 AI160934 |
| | | H79030 AI801493 AA448691 AI673767 AI076042 AI804327 AA813438 |
| | | AA680002 AI274492 T16177 AI287337 AI935050 AA907805 AA911493 |
| | | AI589411 AI371358 AW576236 AI078866 AW516168 AA346372 AI560185 |
| | | AA471009 R75857 AA296025 AA523155 AA853168 AI696593 AI658482 |
| | | AI566601 AW072797 AA128047 AA035502 AW243274 AA992517 R43760 |
| 117156 | 145392_1 | W73853 AA928112 W77887 AW889237 AA148524 AI749182 AI754442 AI338392 |
| | | AI253102 AI079403 AI370541 AI697341 H97538 AW188021 AI927669 W72716 |
| | | AI051402 AI188071 AI335900 N21488 AW770478 W92522 AI691028 AI913512 |
| | | AI144448 W73819 AA604358 N28900 W95221 AI868132 H98465 AA148793 |
| 125565 | 1704098_1 | R20840 R20839 |
| 132983 | 11922_1 | M30269 NM_002508 X82245 AI078760 AW957003 D78945 M27445 AA650439 |
| | | AL048816 AV660256 AV660347 AA333052 BE295257 T60999 AA383049 |
| | | AW369677 Z26985 AW175704 AA343326 AW747957 AI818389 W17308 W17302 |
| | | H15591 AA371284 AA370412 W94966 BE384365 T28498 R80714 R16959 |
| | | H21723 AW835154 D56097 D56381 W21232 AA190565 AW379755 AW067895 |
| 133681 | 13893_1 | AI352558 Z82248 X78138 NM_003405 AU077248 AA223125 S80794 D78577 |
| | | AI124697 AW403970 BE614089 BE296713 BE621334 L20422 X80536 D54224 |
| | | D54950 X57345 N29226 AA127798 AA340253 F08031 AA192540 H67636 |
| | | AA321827 AW950283 AA084159 BE538808 AW401377 AA256774 C03366 |
| | | W46595 W47608 AA305009 H69431 H69456 AL120082 H11706 AA303717 |
| | | AA361357 H22042 H78020 AW999584 AA134368 AA322911 AA322961 H60980 |
| | | N85248 N31547 H79624 T11718 W85826 AW894663 AW894624 BE167441 |
| | | BE170015 AA304626 AW602163 AW998929 AA156681 AA151067 BE002724 |
| | | AA608688 H82692 BE155392 AW383636 BE155394 AA487004 AW383504 |
| | | AI342365 R82553 W16498 BE155344 AI143938 R69901 AA322873 AW340648 |
| | | R25364 AA367935 AI559406 AA033522 AA374252 AW835019 AI922133 |
| | | AI697089 N99662 AW189078 AI199076 AW151598 W59944 AA662875 W94022 |
| | | AA299055 AI039008 AI829449 AA583503 AI635674 AW131665 AI473820 |
| | | AW273118 AW900930 AA908944 AI688035 AW170272 AI082545 AW468176 |
| | | AI608761 AI082748 AI911682 AI248943 AI831016 AA192465 AI218477 |
| | | AA938406 AA385288 AI809817 AA905196 AI191245 AI470204 AI188296 |
| | | AI421367 AI125315 AI087141 AA629032 AA740589 AI554181 AA150830 |
| | | AI248541 AI077943 AA775958 AA864930 AI261476 AI123121 AI310394 |
| | | AA862331 AA872478 BE537084 AI205606 AA720684 AI872093 AW150042 |
| | | AL120538 AA219627 AA988608 C21397 AI359337 H25337 AI089749 |
| | | AA605146 AI359620 AA150478 AI359738 AW383642 AW995424 AI766457 |
| | | R56892 AI089839 W61343 N69107 W46459 AA565955 N20527 AI279782 |
| | | W46596 AA776573 H23204 AI866231 AI083995 N21530 AA126874 D82630 |
| | | W65437 AI086917 AW382095 AI086877 H69844 AW340217 W85827 L08439 |
| | | AA262704 AA505380 W47413 W94135 AA223241 AW089153 AA084101 BE538000 |
| | | AA096126 T28031 AA491574 R84813 AA774536 AW383522 AA155615 AW383529 |
| | | AA491520 AW028427 AA171496 AI469689 AW664539 AI811102 AI811116 |
| | | BE464590 BE350791 H78021 T15405 H21979 AA219489 H13301 AA505883 |
| | | AI864305 AI423963 AW084401 F04963 R69858 H67097 AI917740 AI655561 |
| | | H69864 AA033631 AW383484 AI886261 H25293 AA513281 AW271187 H11617 |
| | | N79982 AI174338 AI904207 AI904208 BE614558 W94127 W65436 AI272249 |
| | | AA700018 AI579932 AI085941 AW152629 |
| 121335 | 279548_1 | AA404418 AI217248 |
| 130018 | 18986_1 | AA353093 AW957317 AW872498 AI560785 AI289110 AW135512 X97261 |
| | | T68873 |
| 121822 | 244391_1 | AI743860 N49543 AW027759 BE349467 AI656284 BE463975 R35022 |
| | | AA370031 AW955302 AL042109 N53092 AI611424 AL079362 AI969290 |
| | | AI928016 BE394912 BE504220 BE467505 AI611611 AI611407 AI611452 |
| | | W56437 AI284566 AI583349 AW183058 AI308085 AI074952 AA437315 |
| | | AA628161 AW301728 AI150224 AA400137 AA437279 AI223355 AA639462 |
| | | AI261373 AI432414 AI984994 AI539335 AA401550 AA358757 AI609976 |
| | | AA442357 AA359393 AA437046 AA370301 AA429328 AW272055 AI580502 |
| | | AI832944 AI038530 AA425107 AI014986 AI148349 AW237721 AW779756 |
| | | AW137877 AI125293 AA400404 R28554 |
| 108309 | 111495_1 | AA069818 AA069971 AA069923 AA069908 |
| 107832 | genbank_AA021473 | AA021473 |
| 123523 | genbank_AA608588 | AA608588 |
| 123964 | genbank_C13961 | C13961 |
| 118475 | genbank_N66845 | N66845 |
| 104787 | genbank_AA027317 | AA027317 |
| 106596 | 304084_1 | AI583948 AA578212 AW303715 AA653450 AA456981 AI400385 W88533 |
| | | AI224133 AW272145 AA088686 R94698 |
| 113947 | genbank_W84768 | W84768 |
| 108277 | genbank_AA064859 | AA064859 |
| 108427 | genbank_AA076382 | AA076382 |
| 108439 | genbank_AA078986 | AA078986 |
| 131353 | 231290_1 | AW411259 H23555 AW015049 AI684275 AW015886 AW068953 AW014085 |
| | | AI027260 R52686 AA918278 AI129462 AA969360 N34869 AI948416 |
| | | AA534205 AA702483 AA705292 |
| 101447 | entrez_M21305 | M21305 |
| 108931 | genbank_AA147186 | AA147186 |
| 108941 | genbank_AA148650 | AA148650 |
| 103138 | entrez_X65965 | X65965 |
| 119174 | genbank_R71234 | R71234 |
| 119416 | genbank_T97186 | T97186 |
| 105985 | genbank_AA406610 | AA406610 |
| 100327 | entrez_D55640 | D55640 |

Table 2A shows the accession numbers for those pkeys lacking unigeneID's for Table 2. The pkeys in Table 7 lacking unigeneID's are represented within Tables 1-6A. For each probeset we have listed the gene cluster number from which the oligonucleotides were designed. Gene clusters were compiled using sequences derived from Genbank ESTs and mRNAs. These sequences were clustered based on sequence similarity using Clustering and Alignment Tools (DoubleTwist, Oakland California). The Genbank accession numbers for sequences comprising each cluster are listed in the “Accession” column.

TABLE 3

| | |
|---|---|
| Pkey: | Unique Eos probeset identifier number |
| Accession: | Accession number used for previous patent filings |
| ExAccn: | Exemplar Accession number, Genbank accession number |
| UnigeneID: | Unigene number |
| Unigene Title: | Unigene gene title |

| Pkey | Accession | ExAccn | UniGene | UnigeneTitle |
|---|---|---|---|---|
| 100405 | D86425 | AW291587 | Hs.82733 | nidogen 2 |
| 100420 | D86983 | D86983 | Hs 118893 | Melanoma associated gene |
| 100481 | HG1098-HT1098 | X70377 | Hs.121489 | cystatin D |
| 100484 | HG1103-HT1103 | NM_005402 | Hs 288757 | v-ral simian leukemia viral oncogene hom |
| 100718 | HG3342-HT3519 | BE295928 | Hs.75424 | inhibitor of DNA binding 1 , dominant neg |
| 100991 | J03764 | J03836 | Hs.82085 | serine (or cysteine) proteinase inhibito |
| 101097 | L06797 | BE245301 | Hs 89414 | chemokine (C—X—C motif), receptor 4 (fus |
| 101168 | L15388 | NM_005308 | Hs 211569 | G protein-coupled receptor kinase 5 |
| 101194 | L20971 | L20971 | Hs.188 | phosphodiesterase 4B, cAMP-specific (dun |
| 101261 | L35545 | D30857 | Hs.82353 | protein C receptor, endothelial (EPCR) |
| 101345 | L76380 | NM_005795 | Hs.152175 | calcitonin receptor-like |
| 101447 | M21305 | M21305 | | gb: Human alpha satellite and satellite 3 |
| 101485 | M24736 | AA296520 | Hs.89546 | selectin E (endothelial adhesion molecul |
| 101543 | M31166 | M31166 | Hs.2050 | pentaxin-related gene, rapidly induced b |
| 101550 | M31551 | Y00630 | Hs.75716 | serine (or cysteine) proteinase inhibito |
| 101560 | M32334 | AW958272 | Hs.347326 | intercellular adhesion molecule 2 |
| 101674 | M61916 | NM_002291 | Hs.82124 | laminin, beta 1 |
| 101714 | M68874 | M68874 | Hs 211587 | phospholipase A2, group IVA (cytosolic, |
| 101741 | M74719 | NM_003199 | Hs.326198 | transcription factor 4 |
| 101838 | M92934 | BE243845 | Hs.75511 | connective tissue growth factor |
| 101857 | M94856 | BE550723 | Hs.153179 | fatty acid binding protein 5 (psoriasis- |
| 102012 | U03057 | BE259035 | Hs.118400 | singed (Drosophila)-like (sea urchin fas |
| 102024 | U03877 | AA301867 | Hs.76224 | EGF-containing fibulin-like extracellula |
| 102164 | U18300 | NM_000107 | Hs.77602 | damage-specific DNA binding protein 2 (4 |
| 102241 | U27109 | NM_007351 | Hs.268107 | multimerin |
| 102283 | U31384 | AW161552 | Hs.83381 | guanine nucleotide binding protein 11 |
| 102303 | U33053 | U33053 | Hs.2499 | protein kinase C-like 1 |
| 102564 | U59423 | U59423 | Hs 79067 | MAD (mothers against decapentaplegic, Dr |
| 102663 | U70322 | NM_002270 | Hs.168075 | karyopherin (importin) beta 2 |
| 102759 | U81607 | NM_005100 | Hs 788 | A kinase (PRKA) anchor protein (gravin) |
| 102778 | U83463 | AF000652 | Hs.8180 | syndecan binding protein (syntenin) |
| 102804 | U89942 | NM_002318 | Hs.83354 | lysyl oxidase-like 2 |
| 102887 | X04729 | J03836 | Hs 82085 | serine (or cysteine) proteinase inhibito |
| 102898 | X06256 | NM_002205 | Hs.149609 | integrin, alpha 5 (fibronectin receptor, |
| 102915 | X07820 | X07820 | Hs.2258 | matrix metalloproteinase 10 (stromelysin |
| 103036 | X54925 | M13509 | Hs 83169 | matrix metalloproteinase 1 (interstitial |
| 103037 | X54936 | BE018302 | Hs.2894 | placental growth factor, vascular endoth |
| 103095 | X60957 | NM_005424 | Hs.78824 | tyrasine kinase with immunoglobulin and |
| 103158 | X67235 | BE242587 | Hs.118651 | hematopoietically expressed homeobox |
| 103166 | X67951 | AA159248 | Hs.180909 | peroxiredoxin 1 |
| 103185 | X69910 | NM_006825 | Hs.74368 | transmembrane protein (63 kD), endoplasmi |
| 103280 | X79981 | U84722 | Hs 76206 | cadherin 5, type 2, VE-cadherin (vascula |
| 103554 | Z18951 | AI878826 | Hs.74034 | caveolin 1, caveolae protein, 22 kD |
| 103850 | AA187101 | AA187101 | Hs.213194 | hypothetical protein MGC10895 |
| 104465 | N24990 | Z44203 | Hs.26418 | ESTs |
| 104592 | R81003 | AW630488 | Hs.25338 | protease, serine, 23 |
| 104764 | AA025351 | AI039243 | Hs.278585 | ESTs |
| 104786 | AA027168 | AA027167 | Hs.10031 | KIAA0955 protein |
| 104850 | M040465 | AL133035 | Hs.8728 | hypothetical protein DKFZp434G171 |
| 104865 | AA045136 | T79340 | Hs.22575 | B-cell CLL/lymphoma 6, member B (zinc fi |
| 104894 | M054087 | AF065214 | Hs.18858 | phospholipase A2, group IVC (cytosolic, |
| 104952 | AA071089 | AW076098 | Hs.345588 | desmoplakin (DPI, DPII) |
| 104974 | AA085918 | Y12059 | Hs.278675 | bromodomain-containing 4 |
| 105178 | AA187490 | AA313825 | Hs.21941 | AD036 protein |
| 105263 | AA227926 | AW388633 | Hs.6682 | solute carrier family 7, (cationic amino |
| 105330 | AA234743 | AW338625 | Hs.22120 | ESTs |
| 105376 | AA236559 | AW994032 | Hs.8768 | hypothetical protein FLJ10849 |
| 105729 | AA292694 | H46612 | Hs 293815 | Homo sapiens HSPC285 mRNA, partial cds |
| 105826 | AA398243 | AA478756 | Hs.194477 | E3 ubiquitin ligase SMURF2 |
| 105977 | AA406363 | AK001972 | Hs.30822 | hypothetical protein FLJ11110 |
| 106008 | AA411465 | AB033888 | Hs 8619 | SRY (sex determining region Y)-box 18 |
| 106031 | AA412284 | X64116 | Hs.171844 | Homo sapiens cDNA: FLJ22296 fis, clone H |
| 106124 | AA423987 | H93366 | Hs.7567 | Homo sapiens cDNA: FLJ21962 fis, clone H |
| 106155 | AA425309 | AA425414 | Hs 33287 | nuclear factor I/B |
| 106302 | AA435896 | AA398859 | Hs.18397 | hypothetical protein FLJ23221 |
| 106423 | AA448238 | AB020722 | Hs.16714 | Rho guanine exchange factor (GEF) 15 |
| 106793 | AA478778 | H94997 | Hs 16450 | ESTs |
| 107174 | AA621714 | BE122762 | Hs.25338 | ESTs |
| 107216 | D51069 | D51069 | Hs.211579 | melanoma cell adhesion molecule |
| 107295 | T34527 | AA186629 | Hs.80120 | UDP-N-acetyl-alpha-D-galactosamine-polyp |
| 107385 | U97519 | NM_005397 | Hs 16426 | podocalyxin-like |
| 108756 | AA127221 | AA127221 | Hs.117037 | ESTs |
| 108846 | AA132983 | AL117452 | Hs.44155 | DKFZP586G1517 protein |
| 108888 | AA135606 | AA135606 | Hs.189384 | gb: zl10a05.s1 Soares_pregnant_uterus_NbH |
| 109001 | AA156125 | AI056548 | Hs.72116 | hypothetical protein FLJ20992 similar to |
| 109166 | AA179845 | AA219691 | Hs.73625 | RAB6 interacting, kinesin-like (rabkines |
| 109456 | AA232645 | AW956580 | Hs 42699 | ESTs |
| 109768 | F10399 | F06838 | Hs.14763 | ESTs |
| 110107 | H16772 | AW151660 | Hs.31444 | ESTs |
| 110906 | N39584 | AA035211 | Hs.17404 | ESTs |
| 110984 | N52006 | AW613287 | Hs.80120 | UDP-N-acetyl-alpha-D-galactosamine polyp |
| 111006 | N53375 | BE387014 | Hs.166146 | Homer, neuronal immediate early gene, 3 |
| 111018 | N54067 | AI287912 | Hs.3628 | mitogen-activated protein kinase kinase |
| 111133 | N64436 | AW580939 | Hs.97199 | complement component C1q receptor |
| 111760 | R26892 | BE551929 | Hs.268754 | Homo sapiens cDNA FLJ11949 fis, clone HE |
| 113073 | T33637 | N39342 | Hs.103042 | microtubule-associated protein 1B |
| 113195 | T57112 | H83265 | Hs 8881 | ESTs, Weakly similar to S41044 chromosom |
| 113923 | W80763 | AW953484 | Hs 3849 | hypothetical protein FLJ22041 similar to |
| 114521 | AA046808 | AW139036 | Hs.108957 | 40S ribosomal protein S27 isoform |
| 115061 | AA253217 | AI751438 | Hs.41271 | Homo sapiens mRNA full length insert cDN |
| 115096 | AA255991 | AI683069 | Hs.175319 | ESTs |
| 115145 | M258138 | AA740907 | Hs.88297 | ESTs |
| 115819 | AA426573 | AA486620 | Hs.41135 | endomucin-2 |
| 115947 | AA443793 | R47479 | Hs 94761 | KIAA1691 protein |
| 116314 | AA490588 | AI799104 | Hs.178705 | Homo sapiens cDNA FLJ11333 fis, clone PL |
| 116339 | AA496257 | AK000290 | Hs.44033 | dipeptidyl peptidase 8 |
| 116430 | AA609717 | AK001531 | Hs 66048 | hypothetical protein FLJ10669 |
| 116589 | D59570 | AI557212 | Hs 17132 | ESTs, Moderately similar to I54374 gene |
| 116733 | F13787 | AL157424 | Hs.61289 | synaptojanin 2 |
| 117023 | H88157 | AW070211 | Hs.102415 | Homo sapiens mRNA; cDNA DKFZp586N0121 (f |
| 117186 | H98988 | H98988 | Hs.42612 | ESTs, Weakly similar to ALU1_HUMAN ALU S |
| 117563 | N34287 | AF055634 | Hs.44553 | unc5 (C. elegans homolog) c |
| 117997 | N52090 | N52090 | Hs.47420 | EST |
| 118475 | N66845 | N66845 | | gb: za46c11.s1 Soares fetal liver spleen |
| 118581 | N68905 | N68905 | | gb: za69b09.s1 Soares_fetal_lung_NbHL19W |
| 119073 | R32894 | BE245360 | Hs.279477 | ESTs |
| 119155 | R61715 | R61715 | Hs.310598 | ESTs, Moderately similar to ALU1_HUMAN A |
| 119174 | R71234 | R71234 | | gb: yi54c08.s1 Soares placenta Nb2HP Homo |
| 119221 | R98105 | C14322 | Hs 250700 | tryptase beta 1 |
| 119416 | T97186 | T97186 | | gb: ye50h09.s1 Soares fetal liver spleen |
| 119866 | W80814 | AA496205 | Hs.193700 | Homo sapiens mRNA; cDNA DKFZp586l0324 (f |
| 121335 | AA404418 | AA404418 | | gb: zw37e02.s1 Soares_total_fetus_Nb2HF8_{—} |
| 121381 | AA405747 | AW088642 | Hs 97984 | hypothetical protein FLJ22252 similar to |
| 123160 | AA488687 | AA488687 | Hs.284235 | ESTs, Weakly similar to I38022 hypotheti |
| 123473 | AA599143 | AA599143 | | gb: ae52d04.s1 Stratagene lung carcinoma |
| 123523 | AA608588 | AA608588 | | gb: ae54e06.s1 Stratagene lung carcinoma |
| 123533 | AA608751 | AA608751 | | gb: ae56h07.s1 Stratagene lung carcinoma |
| 123964 | C13961 | C13961 | | gb: C13961 Clontech human aorta polyA +mR |
| 124006 | D60302 | AI147155 | Hs.270016 | ESTs |
| 124315 | H94892 | NM_005402 | Hs.288757 | v-ral simian leukemia viral oncogene hom |
| 124659 | N93521 | AI680737 | Hs 289068 | Homo sapiens cDNA FLJ11918 fis, clone HE |
| 124669 | N95477 | AI571594 | Hs.102943 | hypothetical protein MGC12916 |
| 124847 | R60044 | W07701 | Hs.304177 | Homo sapiens clone FLB8503 PR02286 mRNA, |
| 124875 | R70506 | AI887664 | Hs 285814 | sprouty (Drosophila) homolog 4 |
| 125091 | T91518 | T91518 | | gb: ye20f05.s1 Stratagene lung (937210) H |
| 125103 | T95333 | AA570056 | Hs 122730 | ESTs, Moderately similar to KIAA1215 pro |
| 125355 | R45630 | R60547 | Hs 170098 | KIAA0372 gene product |
| 125565 | R20839 | R20840 | | gb: yg05c08.r1 Soares infant brain 1NIB H |
| 125590 | R23858 | R23858 | Hs.143375 | Homo sapiens, clone IMAGE:3840937, mRNA, |
| 126511 | AI024874 | T92143 | Hs.57958 | EGF-TM7-latrophilin-related protein |
| 126563 | W26247 | AA516391 | Hs 181368 | U5 snRNP-specific protein (220 kD), orth |
| 126649 | AA856990 | AA001860 | Hs.279531 | ESTs |
| 126872 | AA136653 | AW450979 | | gb: UI-H-BI3-ala-a-12-0-Ul.s1 NCI_CGAP_Su |
| 127402 | AA358869 | AA358869 | Hs 227949 | SEC13 (S. cerevisiae)-like 1 |
| 127651 | AI123976 | AA382523 | Hs.105689 | MSTP031 protein |
| 127759 | AI369384 | AI369384 | Hs.292441 | ESTs |
| 128062 | AA379500 | AA379621 | Hs 105547 | neural proliferation, differentiation an |
| 128992 | R49693 | H04150 | Hs.107708 | ESTs |
| 129046 | AA195678 | AB029290 | Hs.108258 | actin binding protein; macrophin (microf |
| 129188 | M30257 | NM_001078 | Hs.109225 | vascular cell adhesion molecule 1 |
| 129314 | AA028131 | BE622768 | Hs.290356 | mesoderm development candidate 1 |
| 129371 | M10321 | X06828 | Hs.110802 | von Willebrand factor |
| 129468 | J03040 | AW410538 | Hs.111779 | secreted protein, acidic, cysteine-rich |
| 129765 | M86933 | M86933 | Hs.1238 | amelogenin (Y chromosome) |
| 129805 | AA012933 | AA012848 | Hs.12570 | tubulin-specific chaperone d |
| 129884 | AA286710 | AF055581 | Hs 13131 | lysosomal |
| 130495 | AA243278 | AW250380 | Hs.109059 | mitochondrial ribosomal protein L12 |
| 130639 | D59711 | AI557212 | Hs.17132 | ESTs, Moderately similar to I54374 gene |
| 130657 | T94452 | AW337575 | Hs.201591 | ESTs |
| 130828 | AA053400 | AW631469 | Hs.203213 | ESTs |
| 130972 | AA370302 | D81866 | Hs.21739 | Homo sapiens mRNA; cDNA DKFZp586I1518 (f |
| 131080 | J05008 | NM_001955 | Hs 2271 | endothelin 1 |
| 131137 | U85193 | W27392 | Hs.33287 | nuclear factor I/B |
| 131182 | AA256153 | AI824144 | Hs.23912 | ESTs |
| 131486 | X83107 | F06972 | Hs 27372 | BMX non-receptor tyrosine kinase |
| 131573 | AA046593 | AA040311 | Hs.28959 | ESTs |
| 131647 | AA410480 | AA359615 | Hs.30089 | ESTs |
| 131756 | D45304 | AA443966 | Hs.31595 | ESTs |
| 131859 | M90657 | AW960564 | | transmembrane 4 superfamily member 1 |
| 131881 | AA010163 | AW361018 | Hs.3383 | upstream regulatory element binding prot |
| 132050 | AA136353 | AI267615 | Hs.38022 | ESTs |
| 132083 | Y07867 | BE386490 | Hs.279663 | Pirin |
| 132164 | U84573 | AI752235 | Hs 41270 | procollagen-lysine, 2-oxoglutarate 5-dio |
| 132358 | X60486 | NM_003542 | Hs.46423 | H4 histone family, member G |
| 132413 | AA132969 | AW361383 | Hs.260116 | metalloprotease 1 (pitrilysin family) |
| 132456 | AA114250 | AB011084 | Hs.48924 | KIAA0512 gene product; ALEX2 |
| 132490 | F13782 | NM_001290 | Hs.4980 | LIM domain binding 2 |
| 132676 | AA283035 | N92589 | Hs.261038 | ESTs, Weakly similar to I38022 hypotheti |
| 132687 | AB002301 | AB002301 | Hs.54985 | KIAA0303 protein |
| 132718 | AA056731 | NM_004600 | Hs.554 | Sjogren syndrome antigen A2 (60 kD, ribon |
| 132736 | U68019 | AW081883 | Hs 211578 | Homo sapiens cDNA: FLJ23037 fis, clone L |
| 132760 | H99198 | AA125985 | Hs 56145 | thymosin, beta, identified in neuroblast |
| 132933 | AA598702 | BE263252 | Hs.6101 | hypothetical protein MGC3178 |
| 132968 | N77151 | AF234532 | Hs.61638 | myosin X |
| 132994 | AA505133 | AA112748 | Hs 279905 | clone HQ0310PRO0310p1 |
| 133061 | AB000584 | AI186431 | Hs.296638 | prostate differentiation factor |
| 133147 | D12763 | AA026533 | Hs.66 | interleukin 1 receptor-like 1 |
| 133161 | AA253193 | AW021103 | Hs.6631 | hypothetical protein FLJ20373 |
| 133200 | AA432248 | AB037715 | Hs.183639 | hypothetical protein FLJ10210 |
| 133260 | AA083572 | AA403045 | Hs 6906 | Homo sapiens cDNA. FLJ23197 fis, clone R |
| 133363 | AA479713 | AI866286 | Hs 71962 | ESTs, Weakly similar to B36298 proline-r |
| 133491 | L40395 | BE619053 | Hs 170001 | eukaryotic translation initiation factor |
| 133517 | X52947 | NM_000165 | Hs.74471 | gap junction protein, alpha 1, 43 kD (con |
| 133550 | W80846 | AI129903 | Hs.74669 | vesicle-associated membrane protein 5 (m |
| 133607 | M34539 | BE273749 | | FK506-binding protein 1A (12 kD) |
| 133614 | D67029 | NM_003003 | Hs.75232 | SEC14 (S. cerevisiae)-like 1 |
| 133627 | U09587 | NM_002047 | Hs.75280 | glycyl-tRNA synthetase |
| 133691 | M85289 | M85289 | Hs 211573 | heparan sulfate proteoglycan 2 (perlecan |
| 133696 | D10522 | AI878921 | Hs.75607 | myristoylated alanine-rich protein kinas |
| 133913 | W84712 | AU076964 | Hs.7753 | calumenin |
| 133975 | D29992 | C18356 | Hs.295944 | tissue factor pathway inhibitor 2 |
| 133985 | L34657 | L34657 | Hs.78146 | platelet/endothelial cell adhesion molec |
| 134039 | S78569 | NM_002290 | Hs.78672 | laminin, alpha 4 |
| 134088 | D43636 | AI379954 | Hs.79025 | KIAA0096 protein |
| 134161 | U97188 | AA634543 | Hs.79440 | IGF-II mRNA-binding protein 3 |
| 134299 | AA487558 | AW580939 | Hs.97199 | complement component C1q receptor |
| 134416 | M28882 | X68264 | Hs.211579 | melanoma cell adhesion molecule |
| 134453 | X70683 | AI272141 | Hs.83484 | SRY (sex determining region Y)-box 4 |
| 134656 | X14787 | AI750878 | Hs 87409 | thrombospondin 1 |
| 134989 | AA236324 | AW968058 | Hs 92381 | nudix (nucleoside diphosphate linked moi |
| 135051 | C15324 | AI272141 | Hs 83484 | SRY (sex determining region Y)-box 4 |
| 135073 | AA452000 | W55956 | Hs.94030 | Homo sapiens mRNA; cDNA DKFZp586E1624 (f |
| 135349 | D83174 | AA114212 | Hs.9930 | serine (or cysteine) proteinase inhibito |
| 100114 | D00596 | X02308 | Hs.82962 | thymidylate synthetase |
| 100130 | D11428 | NM_00304 | Hs.103724 | peripheral myelin protein 22 |
| 100143 | D13640 | AU076465 | Hs 278441 | KIAA0015 gene product |
| 100168 | D14874 | H73444 | Hs.394 | adrenomedullin |
| 100208 | D26129 | NM_002933 | Hs.78224 | ribonuclease, RNase A family, 1 (pancrea |
| 100224 | D28476 | AL121516 | Hs.138617 | thyroid hormone receptor interactor 12 |
| 100405 | D86425 | AW291587 | Hs.82733 | nidogen 2 |
| 100420 | D86983 | D86983 | Hs.118893 | Melanoma associated gene |
| 100455 | D87953 | AW888941 | Hs.75789 | N-myc downstream regulated |
| 100529 | HG1862-HT1897 | BE313693 | Hs.334330 | calmodulin 2 (phosphorylase kinase, delt |
| 100618 | HG2614-HT2710 | AI752163 | Hs.114599 | collagen, type VIII, alpha 1 |
| 100619 | HG2639-HT2735 | N24433 | Hs.241567 | RNA binding motif, single stranded inter |
| 100658 | HG2855-HT2995 | U56725 | Hs.180414 | heat shock 70 kD protein 2 |
| 100676 | HG3044-HT3742 | X02761 | Hs 287820 | fibronectin 1 |
| 100718 | HG3342-HT3519 | BE295928 | Hs 75424 | inhibitor of DNA binding 1, dominant neg |
| 100752 | HG3543-HT3739 | T81309 | | insulin-like growth factor 2 (somatomedi |
| 100828 | HG4069-HT4339 | AL048753 | Hs.303649 | small inducible cytokine A2 (monocyte ch |
| 100850 | HG417-HT417 | AA836472 | Hs.297939 | cathepsin B |
| 100991 | J03764 | J03836 | Hs.82085 | serine (or cysteine) proteinase inhibito |
| 101097 | L06797 | BE245301 | Hs 89414 | chemokine (C—X—C motif), receptor 4 (fus |
| 101110 | L08246 | AI439011 | Hs.86386 | myeloid cell leukemia sequence 1 (BCL2-r |
| 101142 | L12711 | L12711 | Hs 89643 | transketolase (Wemicke-Korsakoff syndro |
| 101156 | L13977 | AA340987 | Hs.75693 | prolylcarboxypeptidase (angiotensinase C |
| 101168 | L15388 | NM_005308 | Hs.211569 | G protein-coupled receptor kinase 5 |
| 101184 | L19871 | NM_001674 | Hs 460 | activating transcription factor 3 |
| 101192 | L20859 | BE247295 | Hs.78452 | solute carrier family 20 (phosphate tran |
| 101317 | L42176 | L42176 | Hs.8302 | four and a half LIM domains 2 |
| 101336 | L49169 | NM_006732 | Hs.75678 | FBJ murine osteosarcoma viral oncogene h |
| 101345 | L76380 | NM_005795 | Hs.152175 | calcitonin receptor-like |
| 101400 | M15990 | M15990 | Hs 194148 | v-yes-1 Yamaguchi sarcoma viral oncogene |
| 101475 | M23254 | BE410405 | Hs.76288 | calpain 2, (m/ll) large subunit |
| 101485 | M24736 | AA296520 | Hs.89546 | selectin E (endothelial adhesion molecul |
| 101496 | M26576 | X12784 | Hs.119129 | collagen, type IV, alpha 1 |
| 101505 | M27396 | AA307680 | Hs 75692 | asparagine synthetase |
| 101543 | M31166 | M31166 | Hs 2050 | pentaxin-related gene, rapidly induced b |
| 101557 | M31994 | BE293116 | Hs.76392 | aldehyde dehydrogenase 1 family, member |
| 101560 | M32334 | AW958272 | Hs 347326 | intercellular adhesion molecule 2 |
| 101587 | M35878 | AI752416 | Hs.77326 | insulin-like growth factor binding prote |
| 101592 | M36429 | AF064853 | Hs 91299 | guanine nucleotide binding protein (G pr |
| 101633 | M57730 | NM_004428 | Hs.1624 | ephrin-A1 |
| 101634 | M57731 | AV650262 | Hs.75765 | GR02 oncogene |
| 101667 | M60858 | NM_005381 | | nucleolin |
| 101682 | M62994 | AF043045 | Hs 81008 | filamin B, beta (actin-binding protein-2 |
| 101714 | M68874 | M68874 | Hs 211587 | phospholipase A2, group IVA (cytosolic, |
| 101720 | M69043 | M69043 | Hs 81328 | nuclear factor of kappa light polypeptid |
| 101741 | M74719 | NM_003199 | Hs.326198 | transcription factor 4 |
| 101744 | M75126 | AI879352 | Hs.118625 | hexokinase 1 |
| 101793 | M84349 | W01076 | Hs.278573 | CD59 antigen p18-20 (antigen identified |
| 101837 | M92843 | M92843 | Hs.343586 | zinc finger protein homologous to Zfp-36 |
| 101838 | M92934 | BE243845 | Hs.75511 | connective tissue growth factor |
| 101840 | M93056 | AA236291 | Hs.183583 | serine (or cysteine) proteinase inhibito |
| 101857 | M94856 | BE550723 | Hs.153179 | fatty acid binding protein 5 (psoriasis- |
| 101864 | M95787 | BE392588 | Hs 75777 | transgelin |
| 101931 | S76965 | NM_006823 | Hs.75209 | protein kinase (cAMP-dependent, catalyti |
| 101966 | S81914 | X96438 | Hs.76095 | immediate early response 3 |
| 102012 | U03057 | BE259035 | Hs.118400 | singed (Drosophila)-like (sea urchin fas |
| 102013 | U03100 | BE616287 | Hs.178452 | catenin (cadherin-associated protein), a |
| 102024 | U03877 | AA301867 | Hs.76224 | EGF-containing fibulin-like extracellula |
| 102059 | U08021 | AI752666 | Hs 76669 | nicotinamide N-methyltransferase |
| 102121 | U14391 | NM_004998 | Hs.82251 | myosin IE |
| 102283 | U31384 | AW161552 | Hs.83381 | guanine nucleotide binding protein 11 |
| 102300 | U32944 | AI929721 | Hs.5120 | dynein, cytoplasmic, light polypeptide |
| 102378 | U40369 | AU076887 | Hs 28491 | spermidine/spermine N1-acetyltransferase |
| 102395 | U41767 | AU077005 | Hs 92208 | a disintegrin and metalloproteinase doma |
| 102460 | U48959 | U48959 | Hs.211582 | myosin, light polypeptide kinase |
| 102491 | U51010 | U51010 | | gb: Human nicotinamide N-methyltransferas |
| 102499 | U51478 | BE243877 | Hs 76941 | ATPase, Na+/K+ transporting, beta 3 poly |
| 102523 | U53445 | U53445 | Hs.15432 | downregulated in ovarian cancer 1 |
| 102560 | U59289 | R97457 | Hs 63984 | cadherin 13, H-cadherin (heart) |
| 102564 | U59423 | U59423 | Hs.79067 | MAD (mothers against decapentaplegic, Dr |
| 102589 | U62015 | AU076728 | Hs.8867 | cysteine-rich, angiogenic inducer, 61 |
| 102600 | U63825 | AI984144 | Hs.66713 | hepatitis delta antigen-interacting prot |
| 102645 | U67963 | AL119566 | Hs.6721 | lysosomal |
| 102687 | U73379 | NM_007019 | Hs 93002 | ubiquitin carrier protein E2-C |
| 102693 | U73824 | AA532780 | Hs.183684 | eukaryotic translation initiation factor |
| 102709 | U77604 | AA122237 | Hs.81874 | microsomal glutathione S-transferase 2 |
| 102759 | U81607 | NM_005100 | Hs.788 | A kinase (PRKA) anchor protein (gravin) |
| 102804 | U89942 | NM_002318 | Hs.83354 | lysyl oxidase-like 2 |
| 102882 | X04412 | AI767736 | Hs.290070 | gelsolin (amyloidosis, Finnish type) |
| 102907 | X06985 | BE409861 | Hs.202833 | heme oxygenase (decycling) 1 |
| 102915 | X07820 | X07820 | Hs.2258 | matrix metalloproteinase 10 (stromelysin |
| 102927 | X12876 | BE512730 | Hs.65114 | keratin 18 |
| 102960 | X15729 | AI904738 | Hs.76053 | DEAD/H (Asp-Glu-Ala-Asp/His) box polypep |
| 103011 | X52541 | AJ243425 | Hs.326035 | early growth response 1 |
| 103020 | X53416 | X53416 | Hs.195464 | filamin A, alpha (actin-binding protein- |
| 103029 | X54489 | AW800726 | Hs.789 | GR01 oncogene (melanoma growth stimulati |
| 103036 | X54925 | M13509 | Hs.83169 | matrix metalloproteinase 1 (interstitial |
| 103056 | X57206 | Y18024 | Hs.78877 | inositol 1,4,5-trisphosphate 3-kinase B |
| 103080 | X59798 | AU077231 | Hs 82932 | cyclin D1 (PRAD1 parathyroid adenomatos |
| 103095 | X60957 | NM_005424 | Hs 78824 | tyrosine kinase with immunoglobulin and |
| 103138 | X65965 | X65965 | | gb: H. sapiens SOD-2 gene for manganese su |
| 103176 | X69111 | AL021154 | Hs.76884 | inhibitor of DNA binding 3, dominant neg |
| 103195 | X70940 | AA351647 | Hs 2642 | eukaryotic translation elongation factor |
| 103347 | X87838 | AU077309 | Hs.171271 | catenin (cadherin-associated protein), b |
| 103371 | X91247 | X91247 | Hs.13046 | thioredoxin reductase 1 |
| 103432 | X97748 | X97748 | | gb: H. sapiens PTX3 gene promotor region |
| 103471 | Y00815 | Y00815 | Hs.75216 | protein tyrosine phosphatase, receptor t |
| 103967 | AA303711 | AL120051 | Hs 144700 | ephnn-B1 |
| 104447 | L44538 | AW204145 | Hs.156044 | ESTs |
| 104764 | AA025351 | AI039243 | Hs.278585 | ESTs |
| 104783 | AA027050 | AA533513 | Hs 93659 | protein disulfide isomerase related prot |
| 104798 | AA029462 | AW952619 | Hs.17235 | Homo sapiens clone TCCCIA00176 mRNA sequ |
| 104865 | AA045136 | T79340 | Hs.22575 | B-cell CLL/lymphoma 6, member B (zinc fi |
| 104877 | AA047437 | AI138635 | Hs 22968 | Homo sapiens clone IMAGE 451939, mRNA se |
| 104894 | AA054087 | AF065214 | Hs.18858 | phospholipase A2, group IVC (cytosolic, |
| 104952 | AA071089 | AW076098 | Hs.345588 | desmoplakin (DPI, DPII) |
| 105113 | AA156450 | AB037816 | Hs.8982 | Homo sapiens, clone IMAGE:3506202, mRNA, |
| 105178 | AA187490 | AA313825 | Hs.21941 | AD036 protein |
| 105196 | AA195031 | W84893 | Hs.9305 | angiotensin receptor-like 1 |
| 105215 | AA205724 | AA205759 | Hs.10119 | hypothetical protein FLJ14957 |
| 105263 | AA227926 | AW388633 | Hs 6682 | solute carrier family 7, (cationic amino |
| 105271 | AA227986 | AA807881 | Hs.25329 | ESTs |
| 105330 | AA234743 | AW338625 | Hs.22120 | ESTs |
| 105461 | AA253216 | BE539071 | Hs.69388 | hypothetical protein FLJ20505 |
| 105492 | AA256210 | AI805717 | Hs 289112 | CGI-43 protein |
| 105493 | AA256268 | AL047586 | Hs.10283 | RNA binding motif protein 8B |
| 105594 | AA279397 | AB024334 | Hs.25001 | tyrosine 3-monooxygenase/tryptophan 5-mo |
| 105727 | AA292379 | AL135159 | Hs.20340 | KIAA1002 protein |
| 105732 | AA292717 | AW504170 | Hs.274344 | hypothetical protein MGC12942 |
| 105767 | AA346551 | AW370946 | Hs 23457 | ESTs |
| 105882 | AA400292 | W46802 | Hs.81988 | disabled (Drosophila) homolog 2 (mitogen |
| 105936 | AA404338 | AI678765 | Hs 21812 | ESTs |
| 106031 | AA412284 | X64116 | Hs.171844 | Homo sapiens cDNA: FLJ22296 fis, clone H |
| 106124 | AA423987 | H93366 | Hs.7567 | Homo sapiens cDNA: FLJ21962 fis, clone H |
| 106222 | AA428594 | AA356392 | Hs.21321 | Homo sapiens clone FLB9213 PR02474 mRNA, |
| 106241 | AA430108 | BE019681 | Hs.6019 | Homo sapiens cDNA: FLJ21288 fis, clone C |
| 106263 | AA431462 | W21493 | Hs.28329 | hypothetical protein FLJ14005 |
| 106264 | AA431470 | AL046859 | Hs.3407 | protein kinase (cAMP-dependent, catalyti |
| 106366 | AA443756 | AA186715 | Hs.336429 | RIKEN cDNA 9130422N19 gene |
| 106454 | AA449479 | NM_014038 | Hs.5216 | HSPC028 protein |
| 106634 | AA459916 | W25491 | Hs 288909 | hypothetical protein FLJ22471 |
| 106724 | AA465226 | N48670 | Hs.28631 | Homo sapiens cDNA: FLJ22141 fis, clone H |
| 106793 | AA478778 | H94997 | Hs 16450 | ESTs |
| 106799 | AA479037 | BE313412 | Hs 7961 | Homo sapiens clone 25012 mRNA sequence |
| 106842 | AA482597 | AF124251 | Hs.26054 | novel SH2-containing protein 3 |
| 106868 | AA487561 | BE185536 | Hs.301183 | molecule possessing ankyrin repeats indu |
| 106890 | AA489245 | AA489245 | Hs.88500 | mitogen-activated protein kinase 8 inter |
| 106961 | AA504110 | AW243614 | Hs.18063 | Homo sapiens cDNA FLJ10768 fis, clone NT |
| 106974 | AA520989 | AI817130 | Hs.9195 | Homo sapiens cDNA FLJ13698 fis, clone PL |
| 107030 | AA599434 | AL117424 | Hs.25035 | chloride intracellular channel 4 |
| 107061 | AA608649 | BE147611 | Hs.6354 | stromal cell derived factor receptor 1 |
| 107086 | AA609519 | NM_012331 | Hs 26458 | methionine sulfoxide reductase A |
| 107216 | D51069 | D51069 | Hs.211579 | melanoma cell adhesion molecule |
| 107385 | U97519 | NM_005397 | Hs 16426 | podocalyxin-like |
| 107444 | W28391 | W28391 | Hs.343258 | proliferation-associated 2G4, 38 kD |
| 107985 | AA035638 | T40064 | Hs 71968 | Homo sapiens mRNA, cDNA DKFZp564F053 (fr |
| 108507 | AA083514 | AI554545 | Hs.68301 | ESTs |
| 108695 | AA121315 | AB029000 | Hs.70823 | KIAA1077 protein |
| 108931 | AA147186 | AA147186 | | gb: zo38d01.s1 Stratagene endothelial cel |
| 109001 | AA156125 | AI056548 | Hs.72116 | hypothetical protein FLJ20992 similar to |
| 109195 | AA188932 | AF047033 | Hs.132904 | solute carrier family 4, sodium bicarbon |
| 109390 | AA219653 | AW007485 | Hs.87125 | EH-domain containing 3 |
| 109456 | AA232645 | AW956580 | Hs.42699 | ESTs |
| 109737 | F10078 | AA055415 | Hs.13233 | ESTs, Moderately similar to A47582 B-cel |
| 110411 | H48032 | AW001579 | Hs.9645 | Homo sapiens mRNA for KIAA1741 protein, |
| 110660 | H82117 | AA782114 | Hs.28043 | ESTs |
| 110906 | N39584 | AA035211 | Hs.17404 | ESTs |
| 111018 | N54067 | AI287912 | Hs.3628 | mitogen-activated protein kinase kinase |
| 111091 | N59858 | AA300067 | Hs.33032 | hypothetical protein DKFZp434N185 |
| 111356 | N90933 | BE301871 | Hs.4867 | mannosyl (alpha-1,3-)-glycoprotein beta- |
| 111378 | N93764 | AW160993 | Hs.326292 | hypothetical gene DKFZp434A1114 |
| 111741 | R26124 | AB020653 | Hs 24024 | KIAA0846 protein |
| 111769 | R27957 | AW629414 | Hs.24230 | ESTs |
| 112318 | R55470 | AW083384 | Hs.11067 | ESTs, Highly similar to T46395 hypotheti |
| 112951 | T16550 | AA307634 | Hs.6650 | vacuolar protein sorting 45B (yeast homo |
| 113057 | T26674 | AW194301 | Hs 339283 | Human DNA sequence from clone RP1-187J11 |
| 113195 | T57112 | H83265 | Hs 8881 | ESTs, Weakly similar to S41044 chromosom |
| 113490 | T88700 | BE178110 | Hs.173374 | Homo sapiens cDNA FLJ10500 fis, clone NT |
| 113542 | T90527 | H43374 | Hs.7890 | Homo sapiens mRNA for KIAA1671 protein, |
| 113803 | W42789 | AW880709 | Hs 283683 | chromosome 8 open reading frame 4 |
| 113847 | W60002 | NM_005032 | Hs.4114 | plastin 3 (T isoform) |
| 113910 | W78175 | AA113262 | Hs.17901 | Homo sapiens, clone IMAGE:3937015, mRNA, |
| 113947 | W84768 | W84768 | | gb: zh53d03.s1 Soares_fetal_liver_spleen_{—} |
| 114047 | W94427 | AL035858 | Hs 3807 | FXYD domain-containing ion transport reg |
| 115061 | AA253217 | AI751438 | Hs.41271 | Homo sapiens mRNA full length insert cDN |
| 115819 | AA426573 | AA486620 | Hs.41135 | endomucin-2 |
| 115870 | AA432374 | NM_005985 | Hs.48029 | snail 1 (drosophila homolog), zinc finge |
| 115964 | AA446622 | AA987568 | Hs.74313 | KIAA1265 protein |
| 116228 | AA478771 | AI767947 | Hs 50841 | ESTs |
| 116264 | AA482594 | D51174 | Hs 272239 | lysosomal |
| 116314 | AA490588 | AI799104 | Hs 178705 | Homo sapiens cDNA FLJ11333 fis, clone PL |
| 116589 | D59570 | AI557212 | Hs.17132 | ESTs, Moderately similar to I54374 gene |
| 117023 | H88157 | AW070211 | Hs.102415 | Homo sapiens mRNA; cDNA DKFZp586N0121 (f |
| 117112 | H94648 | AW969999 | Hs 293658 | ESTs |
| 117156 | H97538 | W73853 | | ESTs |
| 117176 | H98670 | H45100 | Hs.49753 | uveal autoantigen with coiled coil domai |
| 117280 | N22107 | M18217 | Hs 172129 | Homo sapiens cDNA: FLJ21409 fis, clone C |
| 119559 | W38197 | W38197 | | Empirically selected from AFFX single pr |
| 119866 | W80814 | AA496205 | Hs.193700 | Homo sapiens mRNA; cDNA DKFZp586l0324 (f |
| 120655 | AA287347 | M305599 | Hs.238205 | hypothetical protein PRO2013 |
| 121314 | AA402799 | W07343 | Hs.182538 | phospholipid scramblase 4 |
| 121335 | AA404418 | AA404418 | | gb: zw37e02 s1 Soares_total_fetus_Nb2HF8_{—} |
| 121822 | AA425107 | AI743860 | | metallothionein 1E (functional) |
| 121835 | AA425435 | AB033030 | Hs.300670 | KIAA1204 protein |
| 122331 | AA42872 | AL133437 | Hs.110771 | Homo sapiens cDNA: FLJ21904 fis, clone H |
| 122577 | AA452860 | AA829725 | Hs.334437 | hypothetical protein MGC4248 |
| 123160 | AA488687 | AA488687 | Hs.284235 | ESTs, Weakly similar to I38022 hypotheti |
| 123486 | AA599674 | BE019072 | Hs.334802 | Homo sapiens cDNA FLJ14680 fis, clone NT |
| 124059 | F13673 | BE387335 | Hs.283713 | ESTs, Weakly similar to S64054 hypotheti |
| 124339 | H99093 | H99093 | Hs 343411 | DEAD/H (Asp-Glu-Ala-Asp/His) box polypep |
| 124358 | N22495 | AW070211 | Hs.102415 | Homo sapiens mRNA; cDNA DKFZp586N0121 (f |
| 124364 | N23031 | AF265555 | Hs.250646 | baculoviral IAP repeat-containing 6 |
| 124726 | R15740 | NM_003654 | Hs.104576 | carbohydrate (keratan sulfate Gal-6) sul |
| 124763 | R39610 | BE410405 | Hs.76288 | calpain 2, (m/ll) large subunit |
| 125167 | W45560 | AL137540 | Hs.102541 | netrin 4 |
| 125304 | Z39833 | AL359573 | Hs.124940 | GTP-binding protein |
| 125307 | Z40583 | AW580945 | Hs.330466 | ESTs |
| 125329 | AA825437 | AA825437 | Hs.58875 | ESTs |
| 125598 | R66613 | T40064 | Hs.71968 | Homo sapiens mRNA; cDNA DKFZp564F053 (fr |
| 125609 | AA868063 | AA868063 | Hs.104576 | carbohydrate (keratan sulfate Gal-6) sul |
| 418245 | AA128075 | AA088767 | Hs 83883 | transmembrane, prostate androgen induced |
| 127435 | N66570 | X69086 | Hs.286161 | Homo sapiens cDNA FLJ13613 fis, clone PL |
| 127566 | AI051390 | AI051390 | Hs.116731 | ESTs |
| 127619 | AA627122 | AA627122 | Hs 163787 | ESTs |
| 128453 | X02761 | X02761 | Hs 287820 | fibronectin 1 |
| 128495 | AF010193 | NM_005904 | Hs.100602 | MAD (mothers against decapentaplegic, Dr |
| 128515 | AA149044 | BE395085 | Hs 10086 | type I transmembrane protein Fn14 |
| 128580 | U82108 | U82108 | Hs.101813 | solute carrier family 9 (sodium/hydrogen |
| 128623 | D78676 | BE076608 | Hs.105509 | CTL2 gene |
| 128642 | L35240 | Z28913 | Hs.102948 | enigma (LIM domain protein) |
| 128669 | AA598737 | W28493 | Hs.180414 | heat shock 70 kD protein 8 |
| 128903 | R69417 | AW150717 | Hs.345728 | STAT induced STAT inhibitors 3 |
| 128914 | AA232837 | AW867491 | Hs.107125 | plasmalemma vesicle associated protein |
| 129087 | N72695 | AI348027 | Hs.108557 | hypothetical protein PP1057 |
| 129188 | M30257 | NM_001078 | Hs.109225 | vascular cell adhesion molecule 1 |
| 129226 | M96843 | BE222494 | Hs.180919 | inhibitor of DNA binding 2, dominant neg |
| 129265 | X68277 | AA530892 | Hs.171695 | dual specificity phosphatase 1 |
| 129345 | AA292440 | R22497 | Hs.110571 | growth arrest and DNA-damage-inducible, |
| 129468 | J03040 | AW410538 | Hs.111779 | secreted protein, acidic, cysteine-rich |
| 129488 | AA228107 | AW966728 | Hs.54642 | methionine adenosyltransferase II, beta |
| 129498 | AA449789 | AA449789 | Hs 75511 | connective tissue growth factor |
| 129557 | W01367 | AL045404 | Hs.46366 | KIAA0948 protein |
| 129619 | AA610116 | AA209534 | Hs.284243 | tetraspan NET-6 protein |
| 129627 | AA258308 | T40064 | Hs 71968 | Homo sapiens mRNA; cDNA DKFZp564F053 (fr |
| 129762 | AA460273 | AA453694 | Hs.12372 | tripartite motif protein TRIM2 |
| 129884 | AA286710 | AF055581 | Hs.13131 | lysosomal |
| 130018 | T68873 | AA353093 | | metallothionein 1L |
| 130147 | D63476 | D63476 | Hs.172813 | PAK-interacting exchange factor beta |
| 130178 | M62403 | U20982 | Hs.1516 | insulin-like growth factor-binding prote |
| 130282 | X55740 | BE245380 | Hs.153952 | 5′ nucleotidase (CD73) |
| 130431 | L10284 | AW505214 | Hs.155560 | calnexin |
| 130495 | AA243278 | AW250380 | Hs.109059 | mitochondrial ribosomal protein L12 |
| 130553 | AA430032 | AF062649 | Hs.252587 | pituitary tumor-transforming 1 |
| 130638 | H16402 | AW021276 | Hs.17121 | ESTs |
| 130639 | D59711 | AI557212 | Hs.17132 | ESTs, Moderately similar to I54374 gene |
| 130657 | T94452 | AW337575 | Hs 201591 | ESTs |
| 130686 | AA431571 | BE548267 | Hs.337986 | Homo sapiens cDNA FLJ10934 fis, clone 0V |
| 130776 | R79356 | AF167706 | Hs.19280 | cysteine-rich motor neuron 1 |
| 130818 | AA280375 | AW190920 | Hs.19928 | hypothetical protein SP329 |
| 130840 | Z49269 | BE048821 | Hs.20144 | small inducible cytokine subfamily A (Cy |
| 130899 | Z41740 | AI077288 | Hs.296323 | serum/glucocorticoid regulated kinase |
| 131002 | AA121543 | AL050295 | Hs.22039 | KIAA0758 protein |
| 131080 | J05008 | NM_001955 | Hs 2271 | endothelin 1 |
| 131084 | AA101878 | NM_017413 | Hs.303084 | apelin, peptide ligand for APJ receptor |
| 131091 | T35341 | AJ271216 | Hs.22880 | dipeptidylpeptidase III |
| 131107 | N87590 | BE620886 | Hs.75354 | GCN1 (general control of amino-acid synt |
| 131182 | AA256153 | AI824144 | Hs.23912 | ESTs |
| 131207 | W74533 | AF104266 | Hs.24212 | latrophilin |
| 131319 | U25997 | NM_003155 | Hs.25590 | stanniocalcin 1 |
| 131328 | V01512 | AW939251 | Hs.25647 | v-fos FBJ murine osteosarcoma viral onco |
| 131328 | V01512 | AW939251 | Hs.25647 | v-fos FBJ murine osteosarcoma viral onco |
| 131328 | V01512 | AW939251 | Hs 25647 | v-fos FBJ murine osteosarcoma viral onco |
| 131328 | V01512 | AW939251 | Hs 25647 | v-fos FBJ murine osteosarcoma viral onco |
| 131509 | X56681 | X56681 | Hs.2780 | jun D proto-oncogene |
| 131555 | AA161292 | T47364 | Hs.278613 | interferon, alpha-inducible protein 27 |
| 131564 | AA491465 | T93500 | Hs.28792 | Homo sapiens cDNA FLJ11041 fis, clone PL |
| 131573 | AA046593 | AA040311 | Hs 28959 | ESTs |
| 131692 | D50914 | BE559681 | Hs.30736 | KIAA01 24 protein |
| 131756 | D45304 | AA443966 | Hs.31595 | ESTs |
| 131859 | M90657 | AW960564 | | transmembrane 4 superfamily member 1 |
| 131909 | W69127 | NM_016558 | Hs.274411 | SCAN domain-containing 1 |
| 131915 | AA316186 | AI161383 | Hs.34549 | ESTs, Highly similar to S94541 1 clone 4 |
| 132046 | AA384503 | AI359214 | Hs.179260 | chromosome 14 open reading frame 4 |
| 132050 | AA136353 | AI267615 | Hs.38022 | ESTs |
| 132151 | AA044755 | BE379499 | Hs.173705 | Homo sapiens cDNA: FLJ22050 fis, clone H |
| 132164 | U84573 | AI752235 | Hs.41270 | procollagen-lysine, 2-oxoglutarate 5-dio |
| 132187 | AA058911 | AA235709 | Hs.4193 | DKFZP586O1624 protein |
| 132303 | AA620962 | BE177330 | Hs 325093 | Homo sapiens cDNA. FLJ21210 fis, clone C |
| 132314 | AA285290 | AF112222 | Hs.323806 | pinin, desmosome associated protein |
| 132358 | X60486 | NM_003542 | Hs.46423 | H4 histone family, member G |
| 132398 | R31641 | AA876616 | Hs.16979 | ESTs, Weakly similar to A43932 mucin 2 p |
| 132421 | AA489190 | AW163483 | Hs 48320 | double ring-finger protein, Dorfin |
| 132490 | F13782 | NM_001290 | Hs.4980 | LIM domain binding 2 |
| 132520 | AA257993 | AA257992 | Hs 50651 | Janus kinase 1 (a protein tyrosine kinas |
| 132546 | M24283 | M24283 | Hs.168383 | intercellular adhesion molecule 1 (CD54) |
| 132610 | AA443114 | AA160511 | Hs.5326 | amino acid system N transporter 2; porcu |
| 132716 | T35289 | BE379595 | Hs 283738 | casein kinase 1, alpha 1 |
| 132840 | N23817 | BE218319 | Hs.5807 | GTPaseRab14 |
| 132883 | AA047151 | AA373314 | Hs.5897 | Homo sapiens mRNA; cDNA DKFZp586P1622 (f |
| 132968 | N77151 | AF234532 | Hs.61638 | myosin X |
| 132989 | AA480074 | AA480074 | Hs.331328 | hypothetical protein FLJ13213 |
| 132999 | Y00787 | Y00787 | Hs.624 | interleukin 8 |
| 133071 | T99789 | BE384932 | Hs.64313 | ESTs, Weakly similar to AF257182 1 G-pro |
| 133076 | W84341 | AW946276 | Hs.6441 | Homo sapiens mRNA; cDNA DKFZp586J021 (fr |
| 133099 | L09209 | W16518 | Hs.279518 | amyloid beta (A4) precursor-like protein |
| 133147 | D12763 | AA026533 | Hs.66 | interleukin 1 receptor-like 1 |
| 133149 | T16484 | AA370045 | Hs.6607 | AXIN1 up-regulated |
| 133161 | AA253193 | AW021103 | Hs.6631 | hypothetical protein FLJ20373 |
| 133200 | AA432248 | AB037715 | Hs.183639 | hypothetical protein FLJ10210 |
| 133220 | X82200 | NM_006074 | Hs.318501 | Homo sapiens mRNA full length insert cDN |
| 133260 | AA083572 | AA403045 | Hs 6906 | Homo sapiens cDNA: FLJ23197 fis, clone R |
| 133295 | L00352 | AI147861 | Hs.213289 | low density lipoprotein receptor (famili |
| 133349 | N75791 | AW631255 | Hs 8110 | L-3-hydroxyacyl-Coenzyme A dehydrogenase |
| 133391 | X57579 | AW103364 | Hs.727 | inhibin, beta A (activin A, activin AB a |
| 133398 | X02612 | NM_000499 | Hs.72912 | cytochrome P450, subfamily I (aromatic c |
| 133436 | H44631 | BE294068 | Hs.737 | immediate early protein |
| 133454 | AA090257 | BE547647 | Hs.177781 | hypothetical protein MGC5618 |
| 133478 | X83703 | X83703 | Hs.31432 | cardiac ankyrin repeat protein |
| 133491 | L40395 | BE619053 | Hs.170001 | eukaryotic translation initiation factor |
| 133510 | AA227913 | AW880841 | Hs.96908 | p53-induced protein |
| 133517 | X52947 | NM_000165 | Hs.74471 | gap junction protein, alpha 1, 43 kD (con |
| 133526 | M11313 | AU077051 | Hs 74561 | alpha-2-macroglobulin |
| 133538 | L14837 | NM_003257 | Hs.74614 | tight junction protein 1 (zona occludens |
| 133562 | M60721 | M60721 | Hs.74870 | H2.0 (Drosophila)-like homeo box 1 |
| 133584 | D90209 | D90209 | Hs.181243 | activating transcription factor 4 (tax-r |
| 133590 | T67986 | T70956 | Hs.75106 | clusterin (complement lysis inhibitor, S |
| 133617 | AA148318 | BE244334 | Hs.75249 | ADP-ribosylation factor-like 6 interacti |
| 133651 | U97105 | AI301740 | Hs.173381 | dihydropyrimidinase-like 2 |
| 133671 | T25747 | AW503116 | Hs 301819 | zinc finger protein 146 |
| 133678 | K02574 | AW247252 | | nucleoside phosphorylase |
| 133681 | D78577 | AI352558 | | tyrosine 3-monooxygenase/tryptophan 5-mo |
| 133722 | X53331 | AW969976 | Hs.279009 | matrix Gla protein |
| 133730 | S73591 | BE242779 | Hs.179526 | upregulated by 1,25-dihydroxyvitamin D-3 |
| 133750 | X95735 | BE410769 | Hs.75873 | zyxin |
| 133802 | L16862 | AW239400 | Hs.76297 | G protein-coupled receptor kinase 6 |
| 133825 | U44975 | BE616902 | Hs.285313 | core promoter element binding protein |
| 133838 | M97796 | BE222494 | Hs.180919 | inhibitor of DNA binding 2, dominant neg |
| 133859 | U86782 | U86782 | Hs.178761 | 26S proteasome-associated pad1 homolog |
| 133889 | AA099391 | U48959 | Hs.211582 | myosin, light polypeptide kinase |
| 133960 | M19267 | M19267 | Hs 77899 | tropomyosin 1 (alpha) |
| 133975 | D29992 | C18356 | Hs.295944 | tissue factor pathway inhibitor 2 |
| 133977 | L19314 | AL125639 | Hs.250666 | hairy (Drosophila)-homolog |
| 134039 | S78569 | NM_002290 | Hs.78672 | laminin, alpha 4 |
| 134075 | U28811 | NM_012201 | Hs.78979 | Golgi apparatus protein 1 |
| 134081 | L77886 | AL034349 | Hs.79005 | protein tyrosine phosphatase, receptor t |
| 134164 | C14407 | AW245540 | Hs.79516 | brain abundant, membrane attached signal |
| 134203 | M60278 | AA161219 | Hs.799 | diphtheria toxin receptor (heparin-bindi |
| 134238 | R81509 | AA102179 | Hs.160726 | Homo sapiens cDNA FLJ11680 fis, clone HE |
| 134299 | AA487558 | AW580939 | Hs.97199 | complement component C1q receptor |
| 134332 | D86962 | D86962 | Hs 81875 | growth factor receptor-bound protein 10 |
| 134339 | AA478971 | R70429 | Hs.81988 | disabled (Drosophila) homolog 2 (mitogen |
| 134343 | D50683 | D50683 | Hs.82028 | transforming growth factor, beta recepto |
| 134381 | U56637 | AI557280 | Hs.184270 | capping protein (actin filament) muscle |
| 134403 | M61199 | AA334551 | | sperm specific antigen 2 |
| 134416 | M28882 | X68264 | Hs.211579 | melanoma cell adhesion molecule |
| 134493 | X15183 | M30627 | Hs 289088 | heat shock 90 kD protein 1, alpha |
| 134558 | S53911 | NM_001773 | Hs.85289 | CD34 antigen |
| 134817 | U20734 | AU076592 | Hs.198951 | jun B proto-oncogene |
| 134983 | D28235 | D28235 | Hs.196384 | prostaglandin-endoperoxide synthase 2 (p |
| 134989 | AA236324 | AW968058 | Hs.92381 | nudix (nucleoside diphosphate linked moi |
| 135052 | AA148923 | AL136653 | Hs.93675 | decidual protein induced by progesterone |
| 135062 | AA174183 | AK000967 | Hs 93872 | KIAA1682 protein |
| 135069 | AA456311 | AA876372 | Hs.93961 | Homo sapiens mRNA; cDNA DKFZp667D095 (fr |
| 135071 | L08069 | W27190 | Hs 94 | DnaJ (Hsp40) homolog, subfamily A, membe |
| 135073 | AA452000 | W55956 | Hs.94030 | Homo sapiens mRNA; cDNA DKFZp586E1624 (f |
| 135170 | AA282140 | T53169 | Hs 9587 | Homo sapiens cDNA: FLJ22290 fis, clone H |
| 135196 | J02854 | C03577 | Hs.9615 | myosin regulatory light chain 2, smooth |
| 135348 | AA442054 | U80983 | Hs.268177 | phospholipase C, gamma 1 (formerly subty |
| 134404 | AB000450 | AB000450 | Hs.82771 | vaccinia related kinase 2 |
| 439561 | AB002380 | AF180681 | Hs.6582 | Rho guanine exchange factor (GEF) 12 |
| 100082 | AB003103 | AA130080 | Hs.4295 | proteasome (prosome, macropain) 26S subu |
| 132817 | AB004884 | N27852 | Hs.57553 | tousled-like kinase 2 |
| 130150 | AF000573 | BE094848 | Hs.15113 | homogentisate 1,2-dioxygenase (homogenti |
| 100104 | AF008937 | AF008937 | | syntaxin 16 |
| 447973 | AF009301 | AB011169 | Hs.20141 | similar to S. cerevisiae SSM4 |
| 332613 | AF009368 | AF029674 | Hs 173422 | KIAA1605 protein |
| 100113 | D00591 | NM_001269 | Hs.84746 | chromosome condensation 1 |
| 133980 | D00760 | AA294921 | Hs.348024 | v-ral simian leukemia viral oncogene horn |
| 100129 | D11139 | AA469369 | Hs.5831 | tissue inhibitor of metalloproteinase 1 |
| 100154 | D14657 | H60720 | Hs 81892 | KIAA0101 gene product |
| 100169 | D14878 | AL037228 | Hs.82043 | D123 gene product |
| 129718 | D17716 | NM_002410 | Hs 121502 | mannosyl (alpha-1,6-)-glycoprotein beta- |
| 100190 | D21090 | M91401 | Hs.178658 | RAD23 (S. cerevisiae) homolog B |
| 134742 | D26135 | NM_001346 | Hs.89462 | diacylglycerol kinase, gamma (90 kD) |
| 100211 | D26528 | D26528 | Hs.123058 | DEAD/H (Asp-Glu-Ala-Asp/His) box polypep |
| 100238 | D30742 | L24959 | Hs 348 | calcium/calmodulin-dependent protein kin |
| 130283 | D31762 | NM_012288 | Hs.153954 | TRAM-like protein |
| 134237 | D31765 | D31765 | Hs.170114 | KIAA0061 protein |
| 100248 | D31888 | NM_015156 | Hs.78398 | KIAA0071 protein |
| 100256 | D38128 | D25418 | Hs.393 | prostaglandin 12 (prostacyclin) receptor |
| 100262 | D38500 | D38500 | Hs.278468 | postmeiotic segregation increased 2-like |
| 134329 | D38551 | N92036 | Hs.81848 | RAD21 (S. pombe) homolog |
| 100281 | D42087 | AF091035 | Hs.184627 | KIAA0118 protein |
| 100294 | D49396 | AA331881 | Hs 75454 | peroxiredoxin 3 |
| 100327 | D55640 | D55640 | | gb: Human monocyte PABL (pseudoautosomal |
| 100335 | D63391 | AW247529 | Hs.6793 | platelet-activating factor acetylhydrola |
| 134495 | D63477 | D63477 | Hs.84087 | KIAA0143 protein |
| 100338 | D63483 | D86864 | Hs.57735 | acetyl LDL receptor; SREC |
| 135152 | D64015 | M96954 | Hs.182741 | TIA1 cytotoxic granule-associated RNA-bi |
| 134269 | D79990 | NM_014737 | Hs.80905 | Ras association (RalGDS/AF-6) domain fam |
| 100372 | D79997 | NM_014791 | Hs.184339 | KIAA0175 gene product |
| 134304 | D80010 | BE613486 | Hs.81412 | lipin 1 |
| 100394 | D84276 | D84284 | Hs.66052 | CD38 antigen (p45) |
| 100405 | D86425 | AW291587 | Hs.82733 | nidogen 2 |
| 100418 | D86978 | D86978 | Hs.84790 | KIAA0225 protein |
| 133154 | D87012 | D87012 | Hs.194685 | topoisomerase (DNA) III beta |
| 134347 | D87075 | AF164142 | Hs.82042 | solute carrier family 23 (nucleobase tra |
| 444099 | D87432 | D87432 | Hs.10315 | solute carrier family 7 (cationic amino |
| 100438 | D87448 | AA013051 | Hs.91417 | topoisomerase (DNA) II binding protein |
| 134593 | D87845 | NM_000437 | Hs.234392 | platelet-activating factor acetylhydrola |
| 100481 | HG1098-HT1098 | X70377 | Hs.121489 | cystatin D |
| 100552 | HG2167-HT2237 | AA019521 | Hs.301946 | lysosomal |
| 100591 | HG2415-HT2511 | NM_004091 | Hs.231444 | Homo sapiens, Similar to hypothetical pr |
| 100652 | HG2825-HT2949 | BE613608 | Hs.142653 | ret finger protein |
| 100662 | HG2887-HT3031 | AI368680 | Hs 816 | SRY (sex determining region Y)-box 2 |
| 100899 | HG4660-HT5073 | AL039123 | Hs.103042 | microtubule-associated protein 1B |
| 100905 | HG4704-HT5146 | L12260 | Hs.172816 | neuregulin 1 |
| 100945 | HG884-HT884 | AF002225 | Hs.180686 | ubiquitin protein ligase E3A (human papi |
| 100950 | HG919-HT919 | AF128542 | Hs.166846 | polymerase (DNA directed), epsilon |
| 100964 | J00212 | J00212 | | Empirically selected from AFFX single pr |
| 135407 | J04029 | J04029 | Hs 99936 | keratin 10 (epidermolytic hyperkeratosis |
| 130149 | J04031 | AW067805 | Hs.172665 | methylenetetrahydrofolate dehydrogenase |
| 131877 | J04088 | J04088 | Hs.156346 | topoisomerase (DNA) II alpha (170 kD) |
| 101016 | J04543 | J04543 | Hs.78637 | annexin A7 |
| 134786 | L06139 | T29618 | Hs.89640 | TEK tyrosine kinase, endothelial (venous |
| 134100 | L07540 | AA460085 | Hs.171075 | replication factor C (activator 1) 5 (36 |
| 134078 | L08895 | L08895 | Hs.78995 | MADS box transcription enhancer factor 2 |
| 101132 | L11239 | L11239 | Hs.36993 | gastrulation brain homeo box 1 |
| 134849 | L11353 | BE409525 | Hs 902 | neurofibromin 2 (bilateral acoustic neur |
| 332736 | L13773 | Z83689 | Hs.114765 | myeloid/lymphoid or mixed-lineage leukem |
| 101152 | L13800 | AI984625 | Hs 9884 | spindle pole body protein |
| 135397 | L14922 | L14922 | Hs 166563 | replication factor C (activator 1) 1 (14 |
| 432642 | L15189 | BE297635 | Hs.3069 | heat shock 70 kD protein 9B (mortalin-2) |
| 101168 | L15388 | NM_005308 | Hs.211569 | G protein-coupled receptor kinase 5 |
| 421155 | L16895 | H87879 | Hs.102267 | lysyl oxidase |
| 101226 | L27476 | AF083892 | Hs.75608 | tight junction protein 2 (zona occludens |
| 415138 | L27624 | C18356 | Hs.295944 | tissue factor pathway inhibitor 2 |
| 134739 | L32976 | NM_002419 | Hs 89449 | mitogen-activated protein kinase kinase |
| 130155 | L33404 | AA101043 | Hs.151254 | kallikrein 7 (chymotryptic, stratum corn |
| 440538 | L35263 | W76332 | Hs.79107 | mitogen-activated protein kinase 14 |
| 409916 | L37347 | BE313625 | Hs.57435 | solute carrier family 11 (proton-coupled |
| 101294 | L40371 | AF168418 | Hs.116784 | thyroid hormone receptor interactor 4 |
| 101300 | L40391 | BE535511 | | transmembrane trafficking protein |
| 101310 | L41607 | L41607 | Hs 934 | glucosaminyl (N-acetyl) transferase 2, I |
| 130344 | L77566 | AW250122 | Hs.154879 | DiGeorge syndrome critical region gene D |
| 101381 | M13928 | AW675039 | Hs 1227 | aminolevulinate, delta-, dehydratase |
| 101381 | M13928 | AW675039 | Hs 1227 | aminolevulinate, delta-, dehydratase |
| 415678 | M14016 | AW005903 | Hs.78601 | uroporphyrinogen decarboxylase |
| 133780 | M14219 | AA557660 | Hs.76152 | decorin |
| 101396 | M15796 | BE267931 | Hs.78996 | proliferating cell nuclear antigen |
| 101447 | M21305 | M21305 | | gb: Human alpha satellite and satellite 3 |
| 101458 | M22092 | M22092 | | gb: Human neural cell adhesion molecule ( |
| 101470 | M22898 | NM_000546 | Hs.1846 | tumor protein p53 (Li-Fraumeni syndrome) |
| 134604 | M22995 | NM_002884 | Hs.865 | RAP1 A, member of RAS oncogene family |
| 101478 | M23379 | NM_002890 | Hs.758 | RAS p21 protein activator (GTPase activa |
| 133519 | M24400 | AW583062 | Hs.74502 | chymotrypsinogen B1 |
| 131185 | M25753 | BE280074 | Hs.23960 | cyclin B1 |
| 134116 | M27691 | R84694 | Hs.79194 | cAMP responsive element binding protein |
| 133999 | M28213 | AA535244 | Hs.78305 | RAB2, member RAS oncogene family |
| 130174 | M29550 | M29551 | Hs.151531 | protein phosphatase 3 (formerly 2B), cat |
| 129963 | M29971 | M29971 | Hs.1384 | O-6-methylguanine-DNA methyltransferase |
| 132983 | M30269 | M30269 | | nidogen (enactin) |
| 133900 | M31158 | M31158 | Hs 77439 | protein kinase, cAMP-dependent, regulato |
| 101543 | M31166 | M31166 | Hs 2050 | pentaxin-related gene, rapidly induced b |
| 101545 | M31210 | BE246154 | Hs.154210 | endothelial differentiation, sphingolipi |
| 101620 | M55420 | S55271 | Hs.247930 | Epsilon, IgE |
| 134691 | M59979 | AW382987 | Hs.88474 | prostaglandin-endoperoxide synthase 1 (p |
| 133595 | M62810 | AA393273 | Hs 75133 | transcription factor 6-like 1 (mitochond |
| 101700 | M64710 | D90337 | Hs.247916 | natriuretic peptide precursor C |
| 101714 | M68874 | M68874 | Hs.211587 | phospholipase A2, group IVA (cytosolic, |
| 134246 | M74524 | D28459 | Hs.80612 | ubiquitin-conjugating enzyme E2A (RAD6 h |
| 101760 | M80254 | M80254 | Hs.173125 | peptidylprolyl isomerase F (cyclophilin |
| 415022 | M81780 | X59960 | Hs.77813 | sphingomyelin phosphodiesterase 1, acid |
| 415022 | M81780 | X59960 | Hs.77813 | sphingomyelin phosphodiesterase 1, acid |
| 415022 | M81780 | X59960 | Hs.77813 | sphingomyelin phosphodiesterase 1, acid |
| 415022 | M81780 | X59960 | Hs.77813 | sphingomyelin phosphodiesterase 1, acid |
| 415022 | M81780 | X59960 | Hs.77813 | sphingomyelin phosphodiesterase 1, acid |
| 101791 | M83822 | M83822 | Hs.62354 | cell division cycle 4-like |
| 101812 | M86934 | BE439894 | Hs.78991 | DNA segment, numerous copies, expressed |
| 101813 | M87338 | NM_002914 | Hs.139226 | replication factor C (activator 1) 2 (40 |
| 133396 | M96326 | M96326 | Hs 72885 | azurocidin 1 (cationic antimicrobial pro |
| 428161 | M96954 | M96954 | Hs.182741 | TIA1 cytotoxic granule-associated RNA-bi |
| 129026 | M98833 | AL120297 | Hs.108043 | Friend leukemia virus integration 1 |
| 101901 | S66793 | H38026 | Hs 308 | arrestin 3, retinal (X-arrestin) |
| 134831 | S72370 | AA853479 | Hs.89890 | pyruvate carboxylase |
| 134039 | S78569 | NM_002290 | Hs 78672 | laminin, alpha 4 |
| 442355 | S79873 | AA456539 | Hs.8262 | lysosomal-associated membrane protein 2 |
| 101975 | S83325 | AA079717 | Hs 283664 | aspartate beta-hydroxylase |
| 101977 | S83364 | AF112213 | Hs.184062 | putative Rab5-interacting protein |
| 101978 | S83365 | BE561610 | Hs.5809 | putative transmembrane protein; homolog |
| 101998 | U01212 | U01212 | Hs.248153 | olfactory marker protein |
| 102003 | U01922 | U01922 | Hs 125565 | translocase of inner mitochondrial membr |
| 102007 | U02556 | U02556 | Hs 75307 | t-complex-associated-testis-expressed 1- |
| 102009 | U02680 | BE245149 | Hs.82643 | protein tyrosine kinase 9 |
| 416658 | U03272 | U03272 | Hs.79432 | fibrillin 2 (congenital contractural ara |
| 132951 | U04209 | AW821182 | Hs.61418 | microfibrillar-associated protein 1 |
| 135389 | U05237 | U05237 | Hs 99872 | fetal Alzheimer antigen |
| 102048 | U07225 | U07225 | Hs.339 | punnergic receptor P2Y, G-protein coupl |
| 130145 | U07620 | U34820 | Hs.151051 | mitogen-activated protein kinase 10 |
| 303153 | U09759 | U09759 | Hs.246857 | mitogen-activated protein kinase 9 |
| 420269 | U09820 | U72937 | Hs.96264 | alpha thalassemia/mental retardation syn |
| 102095 | U11313 | U11313 | Hs.75760 | sterol carrier protein 2 |
| 102123 | U14518 | NM_001809 | Hs.1594 | centromere protein A (17 kD) |
| 102126 | U14575 | AW950870 | Hs.78961 | protein phosphatase 1, regulatory (inhib |
| 102133 | U15173 | AU076845 | Hs.155596 | BCL2/adenovirus E1B 19 kD-interacting pro |
| 102139 | U15932 | NM_004419 | Hs 2128 | dual specificity phosphatase 5 |
| 102162 | U18291 | AA450274 | Hs.1592 | CDC16 (cell division cycle 16, S. cerevi |
| 102164 | U18300 | NM_000107 | Hs.77602 | damage-specific DNA binding protein 2 (4 |
| 427653 | U18383 | AA159001 | Hs.180069 | nuclear respiratory factor 1 |
| 131817 | U20536 | U20536 | Hs 3280 | caspase 6, apoptosis-related cysteine pr |
| 102200 | U21551 | AA232362 | Hs.157205 | branched chain aminotransferase 1, cytos |
| 102210 | U23028 | BE619413 | Hs.2437 | eukaryotic translation initiation factor |
| 102214 | U23752 | U23752 | Hs.32964 | SRY (sex determining region Y)-box 11 |
| 132811 | U25435 | U25435 | Hs.57419 | CCCTC-binding factor (zinc finger protei |
| 131319 | U25997 | NM_003155 | Hs.25590 | stanniocalcin 1 |
| 102256 | U28251 | U28251 | Hs.53237 | ESTs, Highly similar to Z169_HUMAN ZINC |
| 132316 | U28831 | U28831 | Hs 44566 | KIAA1641 protein |
| 102269 | U30245 | U30245 | | gb: Human myelomonocytic specific protein |
| 417526 | U32315 | AA568906 | Hs.82240 | syntaxin 3A |
| 102293 | U32439 | AF090116 | Hs 79348 | regulator of G-protein signalling 7 |
| 102298 | U32849 | AA382169 | Hs 54483 | N-myc (and STAT) interactor |
| 102325 | U35139 | AI815867 | Hs 50130 | necdin (mouse) homolog |
| 428734 | U36764 | BE303044 | Hs 192023 | eukaryotic translation initiation factor |
| 102361 | U39400 | AA223616 | Hs.75859 | chromosome 11 open reading frame 4 |
| 102367 | U39657 | U39656 | Hs.118825 | mitogen-activated protein kinase kinase |
| 102388 | U41344 | AA362907 | Hs 76494 | proline arginine-rich end leucine-rich r |
| 102394 | U41766 | NM_003816 | Hs 2442 | a disintegrin and metalloproteinase doma |
| 129829 | U41813 | AF010258 | Hs.127428 | homeo box A9 |
| 102409 | U43286 | BE300330 | Hs.118725 | selenophosphate synthetase 2 |
| 133746 | U44378 | AW410035 | Hs 75862 | MAD (mothers against decapentaplegic, Dr |
| 102423 | U44754 | Z47542 | Hs 179312 | small nuclear RNA activating complex, po |
| 132828 | U47011 | AB014615 | Hs.57710 | fibroblast growth factor 8 (androgen-ind |
| 132828 | U47011 | AB014615 | Hs.57710 | fibroblast growth factor 8 (androgen-ind |
| 132828 | U47011 | AB014615 | Hs 57710 | fibroblast growth factor 8 (androgen-ind |
| 132828 | U47011 | AB014615 | Hs.57710 | fibroblast growth factor 8 (androgen-ind |
| 425322 | U47077 | U63630 | Hs 155637 | protein kinase, DMA-activated, catalytic |
| 102450 | U48251 | U48251 | Hs.75871 | protein kinase C binding protein 1 |
| 129350 | U50535 | U50535 | Hs.110630 | Human BRCA2 region, mRNA sequence CG006 |
| 102534 | U56833 | U96759 | Hs.198307 | von Hippel-Lindau binding protein 1 |
| 130457 | U58091 | AB014595 | Hs.155976 | cullin 4B |
| 135065 | U58837 | AA019401 | Hs.93909 | cyclic nucleotide gated channel beta 1 |
| 102560 | U59289 | R97457 | Hs 63984 | cadherin 13, H-cadherin (heart) |
| 102567 | U59863 | U63830 | Hs.146847 | TRAF family member-associated NFKB activ |
| 417173 | U67122 | U61397 | Hs 81424 | ubiquitin-like 1 (sentrin) |
| 102638 | U67319 | U67319 | Hs.9216 | caspase 7, apoptosis-related cysteine pr |
| 132736 | U68019 | AW081883 | Hs.211578 | Homo sapiens cDNA: FLJ23037 fis, clone L |
| 133070 | U69611 | U92649 | Hs.64311 | a disintegrin and metalloproteinase doma |
| 102663 | U70322 | NM_002270 | Hs.168075 | karyophenn (importin) beta 2 |
| 134660 | U73524 | U73524 | Hs.87465 | ATP/GTP-binding protein |
| 102735 | U79267 | AF111106 | Hs.3382 | protein phosphatase 4, regulatory subuni |
| 102741 | U79291 | AW959829 | Hs.83572 | hypothetical protein MGC14433 |
| 130564 | U82671 | U82671 | Hs.36980 | melanoma antigen, family A, 2 |
| 130564 | U82671 | U82671 | Hs.36980 | melanoma antigen, family A, 2 |
| 132164 | U84573 | AI752235 | Hs.41270 | procollagen-lysine, 2-oxoglutarate 5-dio |
| 102823 | U90914 | D85390 | Hs.5057 | carboxypeptidase D |
| 102826 | U91316 | NM_007274 | Hs.8679 | cytosolic acyl coenzyme A thioester hydr |
| 102831 | U91932 | AA262170 | Hs.80917 | adaptor-related protein complex 3, sigma |
| 102846 | U96131 | BE264974 | Hs.6566 | thyroid hormone receptor interactor 13 |
| 129777 | U97018 | U97018 | Hs.12451 | echinoderm microtubule-associated protei |
| 134161 | U97188 | AA634543 | Hs 79440 | IGF-II mRNA-binding protein 3 |
| 134854 | V00503 | J03464 | Hs.179573 | collagen, type I, alpha 2 |
| 429257 | X04327 | AW163799 | Hs.198365 | 2,3-bisphosphoglycerate mutase |
| 413985 | X06389 | AI018666 | Hs.75667 | synaptophysin |
| 419768 | X07496 | T72104 | Hs.93194 | apolipoprotein A-l |
| 102915 | X07820 | X07820 | Hs 2258 | matrix metalloproteinase 10 (stromelysin |
| 134656 | X14787 | AI750878 | Hs 87409 | thrombospondin 1 |
| 413858 | X15525 | NM_001610 | Hs.75589 | acid phosphatase 2, lysosomal |
| 102968 | X16396 | AU076611 | Hs.154672 | methylene tetrahydrofolate dehydrogenase |
| 102971 | X16609 | X16609 | Hs 183805 | ankyrin 1, erythrocytic |
| 134037 | X53586 | AI808780 | Hs.227730 | integrin, alpha 6 |
| 134037 | X53586 | AI808780 | Hs.227730 | integrin, alpha 6 |
| 103023 | X53793 | AW500470 | Hs.117950 | multifunctional polypeptide similar to S |
| 103037 | X54936 | BE018302 | Hs 2894 | placental growth factor, vascular endoth |
| 130282 | X55740 | BE245380 | Hs.153952 | 5′ nucleotidase (CD73) |
| 134542 | X57025 | M14156 | Hs 85112 | insulin-like growth factor 1 (somatomedi |
| 128568 | X60673 | H12912 | Hs 274691 | adenylate kinase 3 |
| 128568 | X60673 | H12912 | Hs 274691 | adenylate kinase 3 |
| 103093 | X60708 | S79876 | Hs.44926 | dipeptidylpeptidase IV (CD26, adenosine |
| 413076 | X62048 | U10564 | Hs.75188 | wee1 (S. pombe) homolog |
| 129063 | X63097 | X63094 | Hs.283822 | Rhesus blood group, D antigen |
| 424460 | X63563 | BE275979 | Hs.296014 | polymerase (RNA) II (DNA directed) polyp |
| 411077 | X64037 | AW977263 | Hs.68257 | general transcription factor IIF, polype |
| 103181 | X69636 | X69636 | Hs.334731 | Homo sapiens, clone IMAGE:3448306, mRNA, |
| 103184 | X69878 | U43143 | Hs 74049 | fms-related tyrosine kinase 4 |
| 103194 | X70649 | NM_004939 | Hs 78580 | DEAD/H (Asp-Glu-Ala-Asp/His) box polypep |
| 103208 | X72841 | AW411340 | Hs.31314 | retinoblastoma-binding protein 7 |
| 129698 | X74987 | BE242144 | Hs.12013 | ATP-binding cassette, sub-family E (OABP |
| 131486 | X83107 | F06972 | Hs 27372 | BMX non-receptor tyrosine kinase |
| 130729 | X84194 | AI963747 | Hs 18573 | acylphosphatase 1, erythrocyte (common) |
| 103334 | X85753 | NM_001260 | Hs 25283 | cyclin-dependent kinase 8 |
| 132645 | X87870 | AI654712 | Hs.54424 | hepatocyte nuclear factor 4, alpha |
| 135094 | X89066 | NM_003304 | Hs 250687 | transient receptor potential channel 1 |
| 103352 | X89398 | H09366 | Hs.78853 | uracil-DNA glycosylase |
| 103352 | X89398 | H09366 | Hs.78853 | uracil-DNA glycosylase |
| 103353 | X89399 | X89399 | Hs.119274 | RAS p21 protein activator (GTPase activa |
| 132173 | X89426 | X89426 | Hs.41716 | endothehal cell-specific molecule 1 |
| 103371 | X91247 | X91247 | Hs.13046 | thioredoxin reductase 1 |
| 131584 | X91648 | AA598509 | Hs.29117 | purine-rich element binding protein A |
| 103376 | X92098 | AL036166 | Hs.323378 | coated vesicle membrane protein |
| 103378 | X92110 | AL119690 | Hs.153618 | HCGVIII-1 protein |
| 128510 | X94703 | X94703 | | RAB28, member RAS oncogene family |
| 103410 | X96506 | AA158294 | Hs.295362 | DR1-associated protein 1 (negative cofac |
| 133490 | X97230 | AF022044 | Hs.274601 | killer cell immunoglobulin-like receptor |
| 332689 | X97230 | AF022044 | Hs.274601 | killer cell immunoglobulin-like receptor |
| 103438 | X98263 | AW175781 | Hs.152720 | M-phase phosphoprotein 6 |
| 103440 | X98296 | X98296 | Hs.77578 | ubiquitin specific protease 9, X chromos |
| 103452 | X99584 | NM_006936 | Hs.85119 | SMT3 (suppressor of mif two 3, yeast) ho |
| 133536 | Y00264 | W25797.comp | Hs.177486 | amyloid beta (A4) precursor protein (pro |
| 420234 | Y07566 | AW404908 | Hs 96038 | Ric (Drosophila)-like, expressed in many |
| 426502 | Y07759 | Y07759 | Hs.170157 | myosin VA (heavy polypeptide 12, myoxin) |
| 134662 | Y07827 | NM_007048 | Hs.284283 | butyrophilin, subfamily 3, member A1 |
| 132083 | Y07867 | BE386490 | Hs.279663 | Pirin |
| 103500 | Y09443 | AW408009 | Hs.22580 | alkylglycerone phosphate synthase |
| 134389 | Y09858 | Y09858 | Hs.82577 | spindlin-like |
| 132084 | Y12394 | NM_002267 | Hs 3886 | karyopherin alpha 3 (importin alpha 4) |
| 103540 | Z11559 | NM_002197 | Hs.154721 | aconitase 1, soluble |
| 133152 | Z11695 | Z11695 | Hs.324473 | mitogen-activated protein kinase 1 |
| 103548 | Z15005 | Z15005 | Hs.75573 | centromere protein E (312 kD) |
| 103612 | Z46261 | BE336654 | Hs.70937 | H3 histone family, member A |
| 129092 | AA011243 | D56365 | Hs.63525 | poly(rC)-binding protein 2 |
| 103692 | AA018418 | AW137912 | Hs.227583 | Homo sapiens chromosome X map Xp11.23 L- |
| 103695 | AA018758 | AW207152 | Hs.186600 | ESTs |
| 129796 | AA018804 | BE218319 | Hs.5807 | GTPase Rab14 |
| 434993 | AA031993 | AA306325 | Hs.4311 | SUMO-1 activating enzyme subunit 2 |
| 132683 | AA044217 | BE264633 | Hs.143638 | WD repeat domain 4 |
| 131887 | AA046548 | W17064 | Hs.332848 | SWI/SNF related, matrix associated, acti |
| 103723 | AA057447 | BE274312 | Hs.214783 | Homo sapiens cDNA FLJ14041 fis, clone HE |
| 453368 | AA058376 | W20296 | Hs.288178 | Homo sapiens cDNA FLJ11968 fis, clone HE |
| 133260 | AA083572 | AA403045 | Hs.6906 | Homo sapiens cDNA: FLJ23197 fis, clone R |
| 103765 | AA085696 | AA085696 | Hs.169600 | KIAA0826 protein |
| 103766 | AA088744 | AI920783 | Hs.191435 | ESTs |
| 103767 | AA089688 | BE244667 | | CGI-100 protein |
| 132051 | AA091284 | AA393968 | Hs.180145 | HSPC030 protein |
| 103773 | AA092700 | AI219323 | Hs.101077 | ESTs, Weakly similar to T22363 hypotheti |
| 135289 | AA092968 | AW372569 | Hs.9788 | hypothetical protein MGC10924 similar to |
| 409659 | AA094800 | AW970843 | Hs 55682 | eukaryotic translation initiation factor |
| 103794 | AA100219 | AF244135 | Hs 30670 | hepatocellular carcinoma-associated anti |
| 131471 | AA114885 | AA164842 | Hs.192619 | KIAA1600 protein |
| 134319 | AA129547 | BE304999 | Hs.285754 | fumarate hydratase |
| 103807 | AA133016 | AW958264 | Hs.103832 | similar to yeast Upf3, variant B |
| 446392 | AA149507 | AF142419 | Hs.15020 | homolog of mouse quaking QKI (KH domain |
| 129863 | AA151005 | BE379765 | Hs.129872 | sperm associated antigen 9 |
| 103850 | AA187101 | AA187101 | Hs.213194 | hypothetical protein MGC10895 |
| 103855 | AA195179 | W02363 | | hypothetical protein FLJ10330 |
| 103861 | AA206236 | AA206236 | Hs.4944 | hypothetical protein FLJ12783 |
| 130634 | AA227621 | AI769067 | Hs.127824 | ESTs, Weakly similar to T28770 hypotheti |
| 447735 | AA248283 | AA775268 | Hs.6127 | Homo sapiens cDNA: FLJ23020 fis, clone L |
| 103909 | AA249611 | AA249611 | Hs.47438 | SH3 domain binding glutamic acid-rich pr |
| 458928 | AA282640 | AF043117 | Hs 24594 | ubiquitination factor E4B (homologous to |
| 415824 | AA287199 | D42039 | Hs.78871 | mesoderm development candidate 2 |
| 129013 | AA313990 | AA371156 | Hs.107942 | DKFZP564M112 protein |
| 129435 | AA314256 | AF151852 | Hs.111449 | CGI-94 protein |
| 103988 | AA314389 | AA314389 | Hs.342849 | ADP-ribosylation factor-like 5 |
| 104000 | AA324364 | AI146527 | Hs.80475 | polymerase (RNA) II (DNA directed) polyp |
| 425284 | AA329211 | AF155568 | Hs 348043 | NS1 -associated protein 1 |
| 128629 | AA399187 | AL096748 | Hs.102708 | DKFZP434A043 protein |
| 133281 | AA421079 | AK001601 | Hs.69594 | high-mobility group 20A |
| 104104 | AA422029 | AA422029 | Hs.143640 | ESTs, Weakly similar to hyperpolarizatio |
| 332455 | AA425230 | NM_005754 | Hs.220689 | Ras-GTPase-activating protein SH3-domain |
| 132091 | AA447052 | AW954243 | | KIAA0251 protein |
| 135073 | AA452000 | W55956 | Hs.94030 | Homo sapiens mRNA; cDNA DKFZp586E1624 (f |
| 131367 | AA456687 | AI750575 | Hs.173933 | nuclear factor I/A |
| 129593 | AA487015 | AI338247 | Hs 98314 | Homo sapiens mRNA; cDNA DKFZp586L0120 (f |
| 133505 | C01527 | AI630124 | Hs.324504 | Homo sapiens mRNA, cDNA DKFZp586J0720 (f |
| 132064 | C01714 | AA121098 | Hs 3838 | serum-inducible kinase |
| 442351 | C01811 | W52642 | Hs.8261 | hypothetical protein FLJ22393 |
| 131427 | C02352 | AF151879 | Hs 26706 | CGI-121 protein |
| 433892 | C02375 | AI929357 | Hs.323966 | Homo sapiens clone H63 unknown mRNA |
| 104282 | C14448 | C14448 | Hs 332338 | EST |
| 134827 | D16611 | BE314037 | Hs.89866 | coproporphyrinogen oxidase (coproporphyr |
| 425330 | D25216 | D25216 | Hs.155650 | KIAA0014 gene product |
| 131742 | D31352 | AA961420 | Hs.31433 | ESTs |
| 456935 | D58024 | AA370362 | Hs 57958 | EGF-TM7-latrophilin-related protein |
| 425218 | D80897 | NM_014909 | Hs 155182 | KIAA1036 protein |
| 104334 | D82614 | D82614 | Hs.78771 | phosphoglycerate kinase 1 |
| 134593 | D87845 | NM_000437 | Hs.234392 | platelet-activating factor acetylhydrola |
| 134731 | D89377 | D89377 | Hs 89404 | msh (Drosophila) homeo box homolog 2 |
| 445776 | H06583 | NM_001310 | Hs.13313 | cAMP responsive element binding protein- |
| 131670 | H40732 | H03514 | Hs.15589 | ESTs |
| 104394 | H46617 | AA129551 | Hs.172129 | Homo sapiens cDNA: FLJ21409 fis, clone C |
| 104402 | H56731 | H56731 | Hs.132956 | ESTs |
| 439130 | H75570 | AA306090 | Hs 124707 | ESTs |
| 129077 | H78886 | N74724 | Hs.108479 | ESTs |
| 104417 | H81241 | AI819448 | Hs.320861 | Kruppel-like factor 8 |
| 134927 | L36531 | L36531 | Hs.91296 | integrin, alpha 8 |
| 129280 | M63154 | M63154 | Hs.110014 | gastric intrinsic factor (vitamin B synt |
| 134498 | M63180 | AW246273 | Hs.84131 | threonyl-tRNA synthetase |
| 104460 | M91504 | AW955705 | Hs.62604 | Homo sapiens, clone IMAGE:4299322, mRNA, |
| 104488 | N56191 | N56191 | Hs.106511 | protocadherin 17 |
| 131248 | N78483 | AI038989 | Hs 332633 | Bardet-Biedl syndrome 2 |
| 130017 | R14652 | AK000096 | Hs.143198 | inhibitor of growth family, member 3 |
| 104530 | R20459 | AK001676 | Hs 12457 | hypothetical protein FLJ10814 |
| 104534 | R22303 | R22303 | | gb: yh26b09.r1 Soares placenta Nb2HP Homo |
| 104544 | R33779 | AI091173 | Hs 222362 | ESTs, Weakly similar to p40 [H. sapiens] |
| 133328 | R36553 | AW452738 | Hs.265327 | hypothetical protein DKFZp761I141 |
| 104567 | R64534 | AA040620 | Hs 5672 | hypothetical protein AF140225 |
| 129575 | R70621 | F08282 | Hs.278428 | progestin induced protein |
| 130776 | R79356 | AF167706 | Hs.19280 | cysteine-rich motor neuron 1 |
| 104599 | R84933 | AW815036 | Hs.151251 | ESTs |
| 104660 | AA007160 | BE298665 | Hs 14846 | Homo sapiens mRNA; cDNA DKFZp564D016 (fr |
| 104667 | AA007234 | AI239923 | Hs.63931 | ESTs |
| 104718 | AA018409 | AI143020 | Hs.36250 | ESTs, Weakly similar to I38022 hypotheti |
| 104764 | AA025351 | AI039243 | Hs 278585 | ESTs |
| 104786 | AA027168 | AA027167 | Hs.10031 | KIAA0955 protein |
| 104787 | AA027317 | AA027317 | | gb: ze97d11.s1 Soares_fetal_heart_NbHH19W |
| 134079 | AA029423 | AK001751 | Hs.171835 | hypothetical protein FLJ10889 |
| 104804 | AA031357 | AI858702 | Hs.31803 | ESTs, Weakly similar to N-WASP [H. sapien |
| 104865 | AA045136 | T79340 | Hs 22575 | B-cell CLL/lymphoma 6, member B (zinc fi |
| 130828 | AA053400 | AW631469 | Hs.203213 | ESTs |
| 104907 | AA055829 | AA055829 | Hs.196701 | ESTs, Weakly similar to ALU1_HUMAN ALU S |
| 104943 | AA065217 | AF072873 | Hs 114218 | frizzled (Drosophila) homolog 6 |
| 105013 | AA116054 | H63789 | Hs.296288 | ESTs, Weakly similar to KIAA0638 protein |
| 105024 | AA126311 | AA126311 | Hs.9879 | ESTs |
| 132592 | AA129390 | AW803564 | Hs.288850 | Homo sapiens cDNA: FLJ22528 fis, clone H |
| 105038 | AA130273 | AW503733 | Hs.9414 | KIAA1488 protein |
| 105077 | AA142919 | W55946 | Hs 234863 | Homo sapiens cDNA FLJ12082 fis, clone HE |
| 105096 | AA150205 | AL042506 | Hs.21599 | Kruppel-like factor 7 (ubiquitous) |
| 129215 | AA176867 | AB040930 | Hs.126085 | KIAA1497 protein |
| 105169 | AA180321 | BE245294 | Hs.180789 | S164 protein |
| 132796 | AA180487 | NM_006283 | Hs.173159 | transforming, acidic coiled-coil contain |
| 427210 | AA187634 | BE396283 | Hs.173987 | eukaryotic translation initiation factor |
| 105200 | AA195399 | AA328102 | Hs 24641 | cytoskeleton associated protein 2 |
| 130114 | AA234717 | AA233393 | Hs.14992 | hypothetical protein FLJ11151 |
| 105330 | AA234743 | AW338625 | Hs.22120 | ESTs |
| 105337 | AA234957 | AI468789 | Hs.347187 | myotubularin related protein 1 |
| 422040 | AA235604 | AA172106 | Hs.110950 | Rag C protein |
| 105376 | AA236559 | AW994032 | Hs.8768 | hypothetical protein FLJ10849 |
| 105397 | AA242868 | AA814807 | Hs.7395 | hypothetical protein FLJ23182 |
| 431679 | AA251776 | AK000046 | Hs 343877 | hypothetical protein FLJ20039 |
| 131991 | AA251909 | AF053306 | Hs.36708 | budding uninhibited by benzimidazoles 1 |
| 421305 | AA252672 | BE397354 | Hs.324830 | diptheria toxin resistance protein requi |
| 105489 | AA256157 | AA256157 | Hs.24115 | Homo sapiens cDNA FLJ14178 fis, clone NT |
| 105508 | AA256680 | AA173942 | Hs.326416 | Homo sapiens mRNA; cDNA DKFZp564H1916 (f |
| 105539 | AA258873 | AB040884 | Hs.109694 | KIAA1451 protein |
| 135172 | AA262727 | AB028956 | Hs.12144 | KIAA1033 protein |
| 131569 | AA281451 | AL389951 | Hs 271623 | nucleoporin 50 kD |
| 431129 | AA281545 | AL137751 | Hs.263671 | Homo sapiens mRNA; cDNA DKFZp434I0812 (f |
| 105643 | AA282069 | BE621719 | Hs.173802 | KIAA0603 gene product |
| 105659 | AA283044 | AA283044 | Hs.25625 | hypothetical protein FLJ11323 |
| 105666 | AA283930 | AA426234 | Hs.34906 | ESTs, Weakly similar to T17210 hypotheti |
| 105674 | AA284755 | AI609530 | Hs 279789 | histone deacetylase 3 |
| 105709 | AA291268 | AI928962 | Hs 26761 | DKFZP586L0724 protein |
| 105722 | AA291927 | AI922821 | Hs 32433 | ESTs |
| 105765 | AA343514 | AA299688 | Hs.24183 | ESTs |
| 115951 | AA398109 | BE546245 | Hs.301048 | sec13-like protein |
| 130884 | AA398109 | BE546245 | Hs.301048 | sec13-like protein |
| 105962 | AA405737 | AW880358 | Hs.339808 | hypothetical protein FLJ10120 |
| 105985 | AA406610 | AA406610 | | gb: zv15b10.s1 Soares_NhHMPu_S1 Homo sapi |
| 106008 | AA411465 | AB033888 | Hs 8619 | SRY (sex determining region Y)-box 18 |
| 457322 | AA416886 | AI815486 | Hs.243901 | Homo sapiens cDNA FLJ20738 fis, clone HE |
| 134222 | AA424013 | AW855861 | Hs 8025 | Homo sapiens clone 23767 and 23782 mRNA |
| 446954 | AA424148 | AB037850 | Hs.16621 | DKFZP434I116 protein |
| 106141 | AA424558 | AF031463 | Hs.9302 | phosducin-like |
| 447973 | AA424961 | AB011169 | Hs.20141 | similar to S. cerevisiae SSM4 |
| 106157 | AA425367 | W37943 | Hs.34892 | KIAA1323 protein |
| 428314 | AA425921 | AW135049 | Hs.26285 | Homo sapiens cDNA FLJ10643 fis, clone NT |
| 446727 | AA426220 | AB011095 | Hs.16032 | KIAA0523 protein |
| 106196 | AA427735 | AA525993 | Hs 173699 | ESTs, Weakly similar to ALU1_HUMAN ALU S |
| 457714 | AA430673 | AA083764 | | hypothetical protein MGC3178 |
| 133200 | AA432248 | AB037715 | Hs.183639 | hypothetical protein FLJ10210 |
| 106302 | AA435896 | AA398859 | Hs.18397 | hypothetical protein FLJ23221 |
| 106328 | AA436705 | AL079559 | Hs 28020 | KIAA0766 gene product |
| 450534 | AA446561 | AI570189 | Hs 25132 | KIAA0470 gene product |
| 106423 | AA448238 | AB020722 | Hs.16714 | Rho guanine exchange factor (GEF) 15 |
| 439608 | AA449756 | AW864696 | Hs.301732 | hypothetical protein MGC5306 |
| 106477 | AA450303 | R23324 | Hs.41693 | DnaJ (Hsp40) homolog, subfamily B, membe |
| 106503 | AA452411 | AB033042 | Hs.29679 | cofactor required for Sp1 transcriptiona |
| 446999 | AA454566 | AA151520 | | hypothetical protein MGC4485 |
| 106543 | AA454667 | AA676939 | Hs.69285 | neuropilin 1 |
| 442007 | AA456437 | AA301116 | Hs.142838 | nucleolar phosphoprotein Nopp34 |
| 106589 | AA456646 | AK000933 | Hs.28661 | Homo sapiens cDNA FLJ10071 fis, clone HE |
| 106593 | AA456826 | AW296451 | Hs 24605 | ESTs |
| 106596 | AA456981 | AA452379 | | ESTs, Moderately similar to ALU7_HUMAN A |
| 423064 | AA458959 | AF265208 | Hs.8740 | SWI/SNF related, matrix associated, acti |
| 106636 | AA459950 | AW958037 | Hs 286 | ribosomal protein L4 |
| 106654 | AA460449 | AW075485 | Hs.286049 | phosphoserine aminotransferase |
| 131353 | AA463910 | AW754182 | | gb: RC2-CT0321-131199-011-c01 CT0321 Homo |
| 106707 | AA464603 | AK000566 | Hs.98135 | hypothetical protein FLJ20559 |
| 452909 | AA464606 | NM_015368 | Hs.30985 | pannexin 1 |
| 106717 | AA465093 | AA600357 | Hs.239489 | TIA1 cytotoxic granule-associated RNA-bi |
| 453141 | AA465692 | AB014548 | Hs.31921 | KIAA0648 protein |
| 106747 | AA476473 | NM_007118 | Hs.171957 | triple functional domain (PTPRF interact |
| 106773 | AA478109 | AA478109 | Hs 188833 | ESTs |
| 106781 | AA478474 | AA330310 | Hs.24181 | ESTs |
| 106817 | AA480889 | D61216 | Hs.18672 | ESTs |
| 106846 | AA485223 | AB037744 | Hs.34892 | KIAA1323 protein |
| 106848 | AA485254 | AA449014 | Hs.121025 | chromosome 11 open reading frame 5 |
| 106856 | AA486183 | W58353 | Hs.285123 | Homo sapiens mRNA full length insert cDN |
| 418699 | AA496936 | BE539639 | Hs.173030 | ESTs, Weakly similar to ALU8_HUMAN ALU S |
| 107001 | AA598589 | AI926520 | Hs.31016 | putative DMA binding protein |
| 442853 | AA598831 | AW021276 | Hs.17121 | ESTs |
| 107054 | AA600150 | AI076459 | Hs.15978 | KIAA1272 protein |
| 107059 | AA608545 | BE614410 | Hs.23044 | RAD51 (S. cerevisiae) homolog (E coli Re |
| 107080 | AA609210 | AL122043 | Hs.19221 | hypothetical protein DKFZp566G1424 |
| 107115 | AA610108 | BE379623 | Hs 27693 | peptidylprolyl isomerase (cyclophilin)-l |
| 107130 | AA620582 | AB033106 | Hs.12913 | KIAA1280 protein |
| 107156 | AA621239 | AA137043 | Hs 9663 | programmed cell death 6-interacting prot |
| 107174 | AA621714 | BE122762 | Hs.25338 | ESTs |
| 130621 | AA621718 | AW513087 | Hs.16803 | LUC7 (S. cerevisiae)-like |
| 107190 | D19673 | AA836401 | Hs 87860 | ESTs |
| 132626 | D25755 | AW504732 | Hs.21275 | hypothetical protein FLJ11011 |
| 107217 | D51095 | AL080235 | Hs.35861 | DKFZP586E1621 protein |
| 332584 | D60272 | AA357879 | Hs 29423 | ESTs; Weakly similar to macrophage lecti |
| 444655 | T08879 | AF088886 | Hs.11590 | cathepsin F |
| 107295 | T34527 | AA186629 | Hs.80120 | UDP-N-acetyl-alpha-D-galactosamine polyp |
| 107299 | T40327 | BE277457 | Hs.30661 | hypothetical protein MGC4606 |
| 107315 | T62771 | AA316241 | Hs 90691 | nucleophosmin/nucleoplasmin 3 |
| 107316 | T63174 | T63174 | Hs.193700 | Homo sapiens mRNA; cDNA DKFZp586l0324 (f |
| 107328 | T83444 | AW959891 | Hs.76591 | KIAA0887 protein |
| 107334 | T93641 | T93597 | Hs.187429 | ESTs |
| 456340 | U48263 | U48263 | Hs 89040 | prepronociceptin |
| 128636 | U49065 | U49065 | Hs.102865 | interleukin 1 receptor-like 2 |
| 129938 | U79300 | AW003668 | Hs.135587 | Human clone 23629 mRNA sequence |
| 107375 | U88573 | BE011845 | Hs.251064 | high-mobility group (nonhistone chromoso |
| 130074 | U93867 | AL038596 | Hs.250745 | polymerase (RNA) III (DNA directed) (62k |
| 107387 | W01094 | D86983 | Hs 118893 | Melanoma associated gene |
| 132036 | W01568 | AL157433 | Hs.37706 | hypothetical protein DKFZp434E2220 |
| 107426 | W26853 | W26853 | Hs.291003 | hypothetical protein MGC4707 |
| 135388 | W27965 | W27965 | Hs 99865 | epimorphin |
| 130419 | W36280 | AF037448 | Hs.155489 | NS1-associated protein 1 |
| 107469 | W47063 | W47063 | Hs 94668 | ESTs |
| 434203 | W79060 | BE262677 | Hs.283558 | hypothetical protein PR01855 |
| 107506 | W88550 | AB028981 | Hs.8021 | KIAA1058 protein |
| 132358 | X60486 | NM_003542 | Hs 46423 | H4 histone family, member G |
| 107522 | X78931 | X78931 | Hs.99971 | zinc finger protein 272 |
| 456495 | Z14077 | NM_003403 | Hs 97496 | YY1 transcription factor |
| 107582 | AA002147 | AA002147 | Hs.59952 | EST |
| 107609 | AA004711 | R75654 | Hs.164797 | hypothetical protein FLJ13693 |
| 107661 | AA010383 | AA010383 | Hs.60389 | ESTs |
| 107714 | AA015761 | AA015761 | Hs.60642 | ESTs |
| 107775 | AA018772 | AW008846 | Hs.60857 | ESTs |
| 107832 | AA021473 | AA021473 | | gb: ze66c11.s1 Soares retina N2b4HR Homo |
| 107859 | AA024835 | AW732573 | Hs 47584 | potassium voltage-gated channel, delayed |
| 107914 | AA027229 | AA027229 | Hs.61329 | ESTs, Weakly similar to T16370 hypotheti |
| 107935 | AA029428 | AA029428 | Hs.61555 | ESTs |
| 410196 | AA035143 | AI936442 | Hs.59838 | hypothetical protein FLJ10808 |
| 131461 | AA035237 | AA992841 | Hs.27263 | KIAA1458 protein |
| 108007 | AA039347 | AA039347 | Hs.61916 | EST |
| 108029 | AA040740 | AA040740 | Hs.62007 | ESTs |
| 108040 | AA041551 | AL121031 | Hs.159971 | SWI/SNF related, matrix associated, acti |
| 108084 | AA045513 | AA058944 | Hs.116602 | Homo sapiens, clone IMAGE:4154008, mRNA, |
| 108088 | AA045745 | AA045745 | Hs 62886 | ESTs |
| 108168 | AA055348 | AI453137 | Hs.63176 | ESTs |
| 130719 | AA056582 | AA679262 | Hs.14235 | hypothetical protein FLJ20008; KIAA1839 |
| 108189 | AA056697 | AW376061 | Hs.63335 | ESTs, Moderately similar to A46010 X-lin |
| 108190 | AA056746 | AA056746 | Hs 63338 | EST |
| 108203 | AA057678 | AW847814 | Hs.289005 | Homo sapiens cDNA: FLJ21532 fis, clone C |
| 108216 | AA058681 | AA524743 | Hs.44883 | ESTs |
| 108217 | AA058686 | AA058686 | Hs.62588 | ESTs |
| 108245 | AA062840 | BE410285 | Hs.89545 | proteasome (prosome, macropain) subunit, |
| 108277 | AA064859 | AA064859 | | gb: zm50f03.s1 Stratagene fibroblast (937 |
| 108280 | AA065069 | AA065069 | | gb: zm12e11.s1 Stratagene pancreas (93720 |
| 108309 | AA069923 | AA069818 | | gb: zm67e03.r1 Stratagene neuroepithelium |
| 108340 | AA070815 | AA069820 | Hs 180909 | peroxiredoxin 1 |
| 108403 | AA075374 | AA075374 | | gb: zm87a01.s1 Stratagene ovarian cancer |
| 108427 | AA076382 | AA076382 | | gb: zm91g08.s1 Stratagene ovarian cancer |
| 108435 | AA078787 | T82427 | Hs.194101 | Homo sapiens cDNA: FLJ20869 fis, clone A |
| 108439 | AA078986 | AA078986 | | gb: zm92h01.s1 Stratagene ovarian cancer |
| 108465 | AA079393 | AA079393 | Hs.3462 | cytochrome c oxidase subunit VIIc |
| 108469 | AA079487 | AA079487 | | gb: zm97f08.s1 Stratagene colon HT29 (937 |
| 108500 | AA083207 | AA083207 | Hs.68270 | EST |
| 108501 | AA083256 | AA083256 | | gb: zn08g12.s1 Stratagene hNT neuron (937 |
| 108533 | AA084415 | AA084415 | | gb: zn06g09.s1 Stratagene hNT neuron (937 |
| 108562 | AA085274 | AA100796 | | gb: zm26c06.s1 Stratagene pancreas (93720 |
| 108589 | AA088678 | AI732404 | Hs.68846 | ESTs |
| 130890 | AA100925 | AI907537 | Hs 76698 | stress-associated endoplasmic reticulum |
| 432645 | AA101255 | D14041 | Hs.347340 | H-2K binding factor-2 |
| 130385 | AA126474 | AW067800 | Hs 155223 | stanniocalcin 2 |
| 108749 | AA127017 | AA127017 | Hs.71052 | ESTs |
| 108807 | AA129968 | AI652236 | Hs.49376 | hypothetical protein FLJ20644 |
| 108808 | AA130240 | AA045088 | Hs.62738 | ESTs |
| 108833 | AA131866 | AF188527 | Hs 61661 | ESTs, Weakly similar to AF174605 1 F-box |
| 108846 | AA132983 | AL117452 | Hs.44155 | DKFZP586G1 51 7 protein |
| 108857 | AA133250 | AK001468 | Hs 62180 | anillin (Drosophila Scraps homolog), act |
| 131474 | AA133583 | L46353 | Hs.2726 | high-mobility group (nonhistone chromoso |
| 108894 | AA135941 | AK001431 | Hs.5105 | hypothetical protein FLJ10569 |
| 108941 | AA148650 | AA148650 | | gb: zo09e06.s1 Stratagene neuroepithelium |
| 108968 | AA151110 | AI304870 | Hs.188680 | ESTs |
| 108996 | AA155754 | AW995610 | Hs.332436 | EST |
| 109001 | AA156125 | AI056548 | Hs 72116 | hypothetical protein FLJ20992 similar to |
| 131183 | AA156289 | AI611807 | Hs 285107 | hypothetical protein FLJ13397 |
| 109019 | AA156997 | AA156755 | Hs.72150 | ESTs |
| 109022 | AA157291 | AA157291 | Hs.21479 | ubinuclein 1 |
| 109023 | AA157293 | AA157293 | Hs.72168 | ESTs |
| 109068 | AA164293 | AA164293 | Hs.72545 | ESTs |
| 109072 | AA164676 | AI732585 | Hs.22394 | hypothetical protein FLJ10893 |
| 426981 | AA167375 | AL044675 | Hs.173081 | KIAA0530 protein |
| 130346 | AA167550 | H05769 | Hs.188757 | Homo sapiens, clone MGC.5564, mRNA, comp |
| 109146 | AA176589 | AA176589 | Hs.142078 | EST |
| 109172 | AA180448 | AA180448 | Hs 144300 | EST |
| 428438 | AA187144 | NM_001955 | Hs.2271 | endothelin 1 |
| 129208 | AA189170 | AI587376 | Hs 109441 | MSTP033 protein |
| 109222 | AA192757 | AA192833 | Hs.333512 | similar to rat myomegalin |
| 109300 | AA205650 | AA418276 | Hs.170142 | ESTs |
| 109481 | AA233342 | AA878923 | Hs.289069 | hypothetical protein FLJ21016 |
| 109485 | AA233472 | BE619092 | Hs.28465 | Homo sapiens cDNA: FLJ21869 fis, clone H |
| 109516 | AA234110 | AI471639 | Hs.71913 | ESTs |
| 109537 | D80981 | AI858695 | Hs 34898 | ESTs |
| 109556 | F01660 | AI925294 | Hs.87385 | ESTs |
| 109577 | F02206 | F02206 | Hs.296639 | Homo sapiens potassium channel subunit ( |
| 109578 | F02208 | F02208 | Hs.27214 | ESTs |
| 109595 | F02544 | AA078629 | Hs 27301 | ESTs |
| 109625 | F03918 | H29490 | Hs.22697 | ESTs |
| 428376 | F04258 | AF119665 | Hs 184011 | pyrophosphatase (inorganic) |
| 109648 | F04600 | H17800 | Hs 7154 | ESTs |
| 109671 | F08998 | R59210 | Hs.26634 | ESTs |
| 109699 | F09605 | H18013 | Hs.167483 | ESTs |
| 109820 | F11115 | AW016809 | Hs 119021 | ESTs |
| 109933 | H06371 | R52417 | Hs.20945 | Homo sapiens clone 24993 mRNA sequence |
| 110014 | H10995 | AL109666 | Hs.7242 | Homo sapiens mRNA full length insert cDN |
| 110039 | H11938 | H11938 | Hs.21907 | histone acetyltransferase |
| 110099 | H16568 | R44557 | Hs 23748 | ESTs |
| 110107 | H16772 | AW151660 | Hs 31444 | ESTs |
| 110155 | H18951 | AI559626 | Hs.93522 | Homo sapiens mRNA for KIAA1647 protein, |
| 110197 | H20859 | AW090386 | Hs.112278 | arrestin, beta 1 |
| 110223 | H23747 | H19836 | Hs.31697 | ESTs |
| 110306 | H38087 | H38087 | Hs.105509 | CTL2 gene |
| 110335 | H40331 | H65490 | Hs.18845 | ESTs |
| 110342 | H40567 | H40961 | Hs.33008 | ESTs |
| 110395 | H46966 | AA025116 | Hs.33333 | ESTs |
| 110511 | H56640 | H56640 | Hs.221460 | ESTs |
| 110523 | H57154 | AI040384 | Hs.19102 | ESTs, Weakly similar to organic anion tr |
| 110715 | H96712 | H96712 | Hs.269029 | ESTs |
| 110754 | N20814 | AW302200 | Hs.6336 | KIAA0672 gene product |
| 428454 | N25249 | U55936 | Hs.184376 | synaptosomal-associated protein, 23 kD |
| 431663 | N27100 | NM_016569 | Hs 267182 | TBX3-iso protein |
| 134263 | N39616 | AW973443 | Hs.8086 | RNA (guanine-7-) methyltransferase |
| 110938 | N48982 | N48982 | Hs 38034 | Homo sapiens cDNA FLJ12924 fis, clone NT |
| 110983 | N51957 | NM_015367 | Hs.10267 | MIL1 protein |
| 111081 | N59435 | AI146349 | Hs.271614 | CGI-112 protein |
| 111128 | N64139 | AW505364 | Hs.19074 | LATS (large tumor suppressor, Drosophila |
| 431548 | N66981 | AI834273 | Hs.9711 | novel protein |
| 111216 | N68640 | AW139408 | Hs.152940 | ESTs |
| 437562 | N69352 | AB001636 | Hs.5683 | DEAD/H (Asp-Glu-Ala-Asp/His) box polypep |
| 111399 | R00138 | AW270776 | Hs.18857 | ESTs |
| 111514 | R07998 | R07998 | | gb: yf16g11.s1 Soares fetal liver spleen |
| 428744 | R08929 | BE267033 | Hs.192853 | ubiquitin-conjugating enzyme E2G 2 (homo |
| 111574 | R10307 | AI024145 | Hs.188526 | ESTs |
| 111804 | R33354 | AA482478 | Hs 181785 | ESTs |
| 111831 | R36083 | R36095 | Hs.268695 | ESTs |
| 426773 | R37938 | NM_015556 | Hs 172180 | KIAA0440 protein |
| 111904 | R39330 | Z41572 | | gb: HSCZYB122 normalized infant brain cDN |
| 428371 | R40816 | AB012193 | Hs.183874 | cullin 4A |
| 112033 | R43162 | R49031 | Hs.22627 | ESTs |
| 130987 | R45698 | BE613269 | Hs.21893 | hypothetical protein DKFZp761N0624 |
| 112300 | R54554 | H24334 | Hs 26125 | ESTs |
| 112513 | R68425 | R68425 | Hs 13809 | hypothetical protein FLJ10648 |
| 112514 | R68568 | R68568 | Hs.183373 | src homology 3 domain-containing protein |
| 112522 | R68763 | R68857 | Hs.265499 | ESTs |
| 112540 | R70467 | R69751 | | gb: yi40a10 s1 Soares placenta Nb2HP Homo |
| 428655 | R73565 | H05769 | Hs 188757 | Homo sapiens, clone MGC:5564, mRNA, comp |
| 129534 | R73640 | AK002126 | Hs.11260 | hypothetical protein FLJ11264 |
| 112597 | R78376 | R78376 | Hs 29733 | EST |
| 112732 | R92453 | R92453 | Hs 34590 | ESTs |
| 451798 | T03865 | BE297567 | Hs 27047 | hypothetical protein FLJ20392 |
| 112888 | T03872 | AW195317 | Hs.107716 | hypothetical protein FLJ22344 |
| 131863 | T10072 | AI656378 | Hs.33461 | ESTs |
| 112911 | T10080 | AW732747 | Hs.13493 | like mouse brain protein E46 |
| 132215 | T10132 | AL035703 | Hs.4236 | KIAA0478 gene product |
| 112931 | T15343 | T02966 | Hs.167428 | ESTs |
| 112984 | T23457 | T16971 | Hs.289014 | ESTs, Weakly similar to A43932 mucin 2 p |
| 112998 | T23555 | H11257 | Hs.22968 | Homo sapiens clone IMAGE:451939, mRNA se |
| 133376 | T23670 | BE618768 | Hs.7232 | acetyl-Coenzyme A carboxylase alpha |
| 113026 | T23948 | AA376654 | | eukaryotic translation initiation factor |
| 113070 | T33464 | AB032977 | Hs.6298 | KIAA1151 protein |
| 410781 | T34413 | AI375672 | Hs.165028 | ESTs |
| 113074 | T34611 | AK001335 | Hs.31137 | protein tyrosine phosphatase, receptor t |
| 113095 | T40920 | AA828380 | Hs.126733 | ESTs |
| 113179 | T55182 | BE622021 | Hs.152571 | ESTs, Highly similar to IGF-II mRNA-bind |
| 113337 | T77453 | T77453 | Hs.302234 | ESTs |
| 113421 | T84039 | AI769400 | Hs.189729 | ESTs |
| 113454 | T86458 | AI022166 | Hs 16188 | ESTs |
| 113481 | T87693 | T87693 | Hs.204327 | EST |
| 453345 | T89350 | AA302862 | Hs 90063 | neurocalcin delta |
| 113557 | T90945 | H66470 | Hs.16004 | ESTs |
| 113559 | T90987 | T79763 | Hs.14514 | ESTs |
| 113589 | T91863 | AI078554 | Hs.15682 | ESTs |
| 113591 | T91881 | T91881 | Hs.200597 | KIAA0563 gene product |
| 113619 | T93783 | R08665 | Hs.17244 | hypothetical protein FLJ13605 |
| 113683 | T96687 | AB035335 | Hs.144519 | T-cell leukemia/lymphoma 6 |
| 113692 | T96944 | AL360143 | Hs.17936 | DKFZP434H132 protein |
| 113702 | T97307 | T97307 | | gb: ye53h05.s1 Soares fetal liver spleen |
| 113717 | T97764 | T99513 | Hs.187447 | ESTs |
| 113824 | W48817 | AI631964 | Hs.34447 | ESTs |
| 113840 | W58343 | R72137 | Hs.7949 | DKFZP586B2420 protein |
| 113844 | W59949 | AI369275 | Hs.243010 | Homo sapiens cDNA FLJ14445 fis, clone HE |
| 113902 | W74644 | AA340111 | Hs.100009 | acyl-Coenzyme A oxidase 1, palmitoyl |
| 113904 | W74761 | AF125044 | Hs.19196 | ubiquitin-conjugating enzyme HBUCE1 |
| 113905 | W74802 | R81733 | Hs.33106 | ESTs |
| 113931 | W81205 | BE255499 | Hs.3496 | hypothetical protein MGC15749 |
| 113932 | W81237 | AA256444 | Hs.126485 | hypothetical protein FLJ12604; KIAA1692 |
| 131965 | W90146 | W79283 | Hs.35962 | ESTs |
| 114035 | W92798 | W92798 | Hs.269181 | ESTs |
| 114106 | Z38412 | AW602528 | | gb: RC5-BT0562-260100-011-A02 BT0562 Homo |
| 457308 | Z38709 | AI416988 | Hs.238272 | inositol 1,4,5-triphosphate receptor, ty |
| 114161 | Z38904 | BE548222 | Hs.299883 | hypothetical protein FLJ23399 |
| 424949 | Z39103 | AF052212 | Hs 153934 | core-binding factor, runt domain, alpha |
| 457548 | Z39930 | AW069534 | Hs.279583 | CGI-81 protein |
| 128937 | Z39939 | AA251380 | Hs 10726 | ESTs, Weakly similar to ALU1_HUMAN ALU S |
| 432554 | Z40012 | AI479813 | Hs.278411 | NCK-associated protein 1 |
| 114277 | Z40377 | AI052229 | Hs.25373 | ESTs, Weakly similar to T20410 hypotheti |
| 114304 | Z40820 | AI934204 | Hs.16129 | ESTs |
| 114364 | Z41680 | AL117427 | Hs 172778 | Homo sapiens mRNA; cDNA DKFZp566P013 (fr |
| 432620 | AA005112 | AA777749 | Hs.5978 | LIM domain only 7 |
| 129034 | AA005432 | AA481157 | Hs.108110 | DKFZP547E2110 protein |
| 131881 | AA010163 | AW361018 | Hs 3383 | upstream regulatory element binding prot |
| 332421 | AA026356 | AI909968 | Hs.108106 | transcription factor |
| 114465 | AA026901 | BE621056 | Hs.131731 | hypothetical protein FLJ11099 |
| 451271 | AA036867 | AK001644 | Hs.26156 | hypothetical protein FLJ10782 |
| 332498 | AA044644 | AA303661 | | lymphocyte-specific protein 1 |
| 431555 | AA046426 | AI815470 | Hs.260024 | Cdc42 effector protein 3 |
| 132944 | AA054515 | T96641 | Hs.6127 | Homo sapiens cDNA: FLJ23020 fis, clone L |
| 114618 | AA084162 | AW979261 | Hs.291993 | ESTs |
| 332509 | AA085749 | AA128376 | Hs.153884 | ATP binding protein associated with cell |
| 114648 | AA101056 | AA101056 | | gb: zn25b03.s1 Stratagene neuroepithelium |
| 114658 | AA102746 | AA102383 | Hs 249190 | tumor necrosis factor receptor superfami |
| 132456 | AA114250 | AB011084 | Hs.48924 | KIAA0512 gene product; ALEX2 |
| 450847 | AA126561 | NM_003155 | Hs.25590 | stanniocalcin 1 |
| 132225 | AA128980 | AA128980 | | gb: zo09a11.s1 Stratagene neuroepithelium |
| 437197 | AA129757 | W38586 | | guanine nucleotide binding protein (G pr |
| 114709 | AA129921 | AA397651 | Hs.301959 | proline synthetase co-transcribed (bacte |
| 456926 | AA133331 | AB018284 | Hs.158688 | KIAA0741 gene product |
| 114750 | AA135958 | AA887211 | Hs.129467 | ESTs |
| 426806 | AA136524 | T19228 | Hs.172572 | hypothetical protein FLJ20093 |
| 114763 | AA147044 | AA810755 | Hs.102500 | hypothetical protein dJ511E16.2 |
| 114767 | AA148885 | AI859865 | Hs.154443 | minichromosome maintenance deficient (S. |
| 114774 | AA150043 | AV656017 | Hs.184325 | CGI-76 protein |
| 129388 | AA151621 | AA662477 | Hs.110964 | hypothetical protein FLJ23471 |
| 457742 | AA155743 | BE561824 | Hs.273369 | uncharacterized hematopoietic stem/proge |
| 456200 | AA156335 | AA768242 | Hs.80618 | hypothetical protein |
| 130207 | AA156336 | AF044209 | Hs 144904 | nuclear receptor co-repressor 1 |
| 114798 | AA159181 | AA159181 | Hs 54900 | serologically defined colon cancer antig |
| 114800 | AA159825 | Z19448 | Hs.131887 | ESTs, Weakly similar to T24396 hypotheti |
| 114828 | AA234185 | M252937 | Hs.283522 | Homo sapiens mRNA; cDNA DKFZp434J1912 (f |
| 114846 | AA234929 | BE018682 | Hs.166196 | ATPase, Class I, type 8B, member 1 |
| 114848 | AA234935 | BE614347 | Hs 169615 | hypothetical protein FLJ20989 |
| 114902 | AA236359 | AW275480 | Hs 39504 | hypothetical protein MGC4308 |
| 132271 | AA236466 | AB030034 | Hs.115175 | sterile-alpha motif and leucine zipper c |
| 114907 | AA236535 | N29390 | Hs.13804 | hypothetical protein dJ462O23.2 |
| 420170 | AA236935 | U43374 | Hs 95631 | Human normal keratinocyte mRNA |
| 132204 | AA236942 | AA235827 | Hs.42265 | ESTs |
| 114928 | AA237018 | M237018 | Hs.94869 | ESTs |
| 132481 | AA237025 | W93378 | Hs.49614 | ESTs |
| 114932 | AA242751 | AA971436 | Hs.16218 | KIAA0903 protein |
| 314162 | AA242760 | BE041820 | Hs 38516 | Homo sapiens, clone MGC:15887, mRNA, com |
| 131006 | AA242763 | AF064104 | Hs.22116 | CDC14 (cell division cycle 14, S. cerevi |
| 114935 | AA242809 | H23329 | Hs.290880 | ESTs, Weakly similar to ALU1_HUMAN ALU S |
| 408908 | AA243133 | BE296227 | Hs.250822 | serine/threonine kinase 15 |
| 437754 | AA243495 | R60366 | Hs.5822 | Homo sapiens cDNA FLJ22120 fis, clone H |
| 114957 | AA243706 | AW170425 | Hs.87680 | ESTs |
| 114974 | AA250848 | AW966931 | Hs.302649 | nucleosome assembly protein 1-like 1 |
| 114977 | AA250868 | AW296978 | Hs.87787 | ESTs |
| 114995 | AA251152 | AA769266 | Hs.193657 | ESTs |
| 115005 | AA251544 | AI760825 | Hs 153042 | ESTs |
| 417177 | AA251792 | NM_004458 | Hs.81452 | fatty-acid-Coenzyme A ligase, long-chain |
| 115026 | AA252144 | AA251972 | Hs 188718 | ESTs |
| 115045 | AA252524 | AW014549 | Hs.58373 | ESTs |
| 115068 | AA253461 | AW512260 | Hs.87767 | ESTs |
| 133138 | AA255522 | AV657594 | Hs.181161 | Homo sapiens cDNA FLJ14643 fis, clone NT |
| 332668 | AA255522 | AV657594 | Hs.181161 | ESTs |
| 115114 | AA256468 | AA527548 | Hs 7527 | small fragment nuclease |
| 129584 | AA256528 | AV656017 | Hs.184325 | CGI-76 protein |
| 115137 | AA257976 | AW968304 | Hs 56156 | ESTs |
| 417187 | AA258296 | AB011151 | Hs.334659 | hypothetical protein MGC14139 |
| 115166 | AA258409 | AF095727 | Hs.287832 | myelin protein zero-like 1 |
| 115167 | AA258421 | AA749209 | Hs.43728 | hypothetical protein |
| 436719 | AA262077 | Y11192 | Hs.5299 | aldehyde dehydrogenase 5 family, member |
| 115239 | AA278650 | BE251328 | Hs.73291 | hypothetical protein FLJ10881 |
| 115243 | AA278766 | AA806600 | Hs.116665 | KIAA1842 protein |
| 428419 | AA280791 | U49436 | | KIAA1856 protein |
| 115322 | AA280819 | L08895 | Hs.78995 | MADS box transcription enhancer factor 2 |
| 413303 | AA280828 | AW836130 | Hs.75277 | hypothetical protein FLJ13910 |
| 115372 | AA282195 | AW014385 | Hs.88678 | ESTs, Weakly similar to Unknown [H. sapie |
| 409962 | AA283127 | U82671 | Hs.57698 | Target CAT |
| 130269 | AA284694 | F05422 | Hs.168352 | nucleoporin-like protein 1 |
| 456570 | AA291137 | AA286914 | Hs.183299 | ESTs |
| 332675 | AA291708 | BE439944 | | ESTs |
| 407864 | AA293495 | AF069291 | Hs.40539 | chromosome 8 open reading frame 1 |
| 115536 | AA347193 | AK001468 | Hs.62180 | anillin (Drosophila Scraps homolog), act |
| 408799 | AA398474 | AA059412 | Hs.47986 | hypothetical protein MGC10940 |
| 115575 | AA398512 | AA393254 | Hs 43619 | ESTs |
| 115601 | AA400277 | AA148984 | Hs.48849 | ESTs, Weakly similar to ALU4_HUMAN ALU S |
| 434428 | AA400896 | D14540 | Hs.199160 | myeloid/lymphoid or mixed-lineage leukem |
| 115683 | AA410345 | AF255910 | Hs.54650 | junctional adhesion molecule 2 |
| 115715 | AA416733 | BE395161 | Hs.1390 | proteasome (prosome, macropain) subunit, |
| 132952 | AA425154 | AI658580 | Hs.61426 | Homo sapiens mesenchymal stem cell prate |
| 115819 | AA426573 | AA486620 | Hs.41135 | endomucin-2 |
| 409124 | AA431418 | AW292809 | Hs.50727 | N-acetylglucosaminidase, alpha- (Sanfili |
| 115895 | AA436182 | AB033035 | Hs.51965 | KIAA1209 protein |
| 458073 | AA437099 | AA192669 | Hs.45032 | ESTs |
| 115962 | AA446585 | AI636361 | Hs 179520 | hypothetical protein MGC10702 |
| 115967 | AA446887 | AI745379 | Hs.42911 | ESTs |
| 115974 | AA447224 | BE513442 | Hs.238944 | hypothetical protein FLJ10631 |
| 115985 | AA447709 | AA447709 | Hs 268115 | ESTs, Weakly similar to T08599 probable |
| 129254 | AA453624 | AA252468 | Hs.1098 | DKFZp434J1813 protein |
| 446730 | AA455044 | BE384932 | Hs.64313 | ESTs, Weakly similar to AF257182 1 G-pro |
| 116095 | AA456045 | AA043429 | Hs 62618 | ESTs |
| 426856 | AA460454 | R19768 | Hs.172788 | ALEX3 protein |
| 116210 | AA476494 | BE622792 | Hs.172788 | ALEX3 protein |
| 116213 | AA476738 | AA292105 | Hs 326740 | hypothetical protein MGC10947 |
| 432645 | AA481422 | D14041 | Hs 347340 | H-2K binding factor-2 |
| 116265 | AA482595 | BE297412 | Hs.55189 | hypothetical protein |
| 129334 | AA485084 | AW157022 | Hs 343551 | hypothetical protein FLJ22584 |
| 116274 | AA485431 | AI129767 | Hs.182874 | guanine nucleotide binding protein (G pr |
| 426002 | AA489638 | BE514376 | Hs.165998 | PAI-1 mRNA-binding protein |
| 116331 | AA491000 | N41300 | Hs 71616 | Homo sapiens mRNA; cDNA DKFZp586N1720 (f |
| 116333 | AA491250 | AF155827 | Hs.203963 | hypothetical protein FLJ10339 |
| 132994 | AA505133 | AA112748 | Hs.279905 | clone HQ0310 PRO0310p1 |
| 418538 | AA598447 | BE244323 | Hs.85951 | exportin, tRNA (nuclear export receptor |
| 116391 | AA599243 | T86558 | Hs 75113 | general transcription factor IIIA |
| 116394 | AA599574 | NM_006033 | Hs.65370 | lipase, endothelial |
| 134531 | AA600153 | AI742845 | Hs.110713 | DEK oncogene (DNA binding) |
| 116417 | AA609309 | AW499664 | | Human clone 23826 mRNA sequence |
| 116429 | AA609710 | AF191018 | Hs.279923 | putative nucleotide binding protein, est |
| 116439 | AA610068 | AA251594 | Hs.43913 | PIBF1 gene product |
| 116459 | AA621399 | R80137 | Hs.302738 | Homo sapiens cDNA: FLJ21425 fis, clone C |
| 427505 | AA621752 | AA361562 | Hs.178761 | 26S proteasome-associated pad1 homolog |
| 409633 | C21523 | AW449822 | Hs 55200 | ESTs |
| 116541 | D12160 | D12160 | Hs.249212 | polymerase (RNA) III (DNA directed) (155 |
| 132557 | D19708 | AA114926 | Hs.169531 | ESTs |
| 414964 | D25801 | AA337548 | Hs.333402 | hypothetical protein MGC12760 |
| 116571 | D45652 | D45652 | Hs 211604 | gb: HUMGS02848 Human adult lung 3′ direct |
| 451522 | D60208 | BE565817 | Hs.26498 | hypothetical protein FLJ21657 |
| 421919 | D80504 | AJ224901 | Hs 109526 | zinc finger protein 198 |
| 116643 | F03010 | AI367044 | Hs 153638 | myeloid/lymphoid or mixed-lineage leukem |
| 116661 | F04247 | R61504 | | gb: yh16a03.s1 Soares infant brain 1NIB H |
| 116715 | F10966 | AL117440 | Hs 170263 | tumor protein p53-binding protein, 1 |
| 116729 | F13700 | BE549407 | Hs.115823 | ribonuclease P, 40 kD subunit |
| 318709 | H05063 | R52576 | Hs.285280 | Homo sapiens cDNA: FLJ22096 fis, clone H |
| 418999 | H16758 | NM_000121 | Hs 89548 | erythropoietin receptor |
| 116773 | H17315 | AI823410 | Hs 343581 | karyopherin alpha 1 (importin alpha 5) |
| 116780 | H22566 | H22566 | Hs.63931 | ESTs |
| 453884 | H48459 | AA355925 | Hs.36232 | KIAA0186 gene product |
| 116819 | H53073 | H53073 | Hs 93698 | EST |
| 427278 | H56559 | AL031428 | Hs.174174 | KIAA0601 protein |
| 407833 | H57957 | AW955632 | Hs.66666 | ESTs, Weakly similar to S19560 praline-r |
| 116844 | H64938 | H64938 | Hs.337434 | ESTs, Weakly similar to A46010 X-linked |
| 116845 | H64973 | AA649530 | Hs.348148 | gb: ns44f05.s1 NCl_CGAP_Alv1 Homo sapiens |
| 116892 | H69535 | AI573283 | Hs.38458 | ESTs |
| 116925 | H73110 | H73110 | Hs 260603 | ESTs, Moderately similar to A47582 B-cel |
| 116981 | H81783 | N29218 | Hs 40290 | ESTs |
| 453133 | H86259 | AC005757 | Hs.31809 | hypothetical protein |
| 117031 | H88353 | H88353 | Hs.347265 | gb: yw21a02.s1 Morton Fetal Cochlea Homo |
| 117034 | H88639 | U72209 | | YY1-associated factor 2 |
| 431129 | H88675 | AL137751 | Hs.263671 | Homo sapiens mRNA; cDNA DKFZp434l0812 (f |
| 417861 | H93708 | AA334551 | | sperm specific antigen 2 |
| 117280 | N22107 | M18217 | Hs.172129 | Homo sapiens cDNA: FLJ21409 fis, clone C |
| 117344 | N24046 | R19085 | Hs.210706 | Homo sapiens cDNA FLJ13182 fis, clone NT |
| 117422 | N27028 | AI355562 | Hs.43880 | ESTs, Weakly similar to A46010 X-linked |
| 117475 | N30205 | N30205 | Hs.93740 | ESTs, Weakly similar to I38022 hypotheti |
| 117487 | N30621 | N30621 | Hs.44203 | ESTs |
| 117937 | N33258 | AF044209 | Hs.144904 | nuclear receptor co-repressor 1 |
| 130207 | N33258 | AF044209 | Hs.144904 | nuclear receptor co-repressor 1 |
| 117549 | N33390 | N33390 | Hs.44483 | EST |
| 117683 | N40180 | N40180 | | gb: yy44d02.s1 Soares_multiple_sclerosis_{—} |
| 117710 | N45198 | N45198 | Hs 47248 | ESTs, Highly similar to similar to Cdc14 |
| 117791 | N48325 | N48325 | Hs.93956 | EST |
| 117822 | N48913 | AA706282 | Hs.93963 | ESTs |
| 422544 | N49394 | AB018259 | Hs.118140 | KIAA0716 gene product |
| 117895 | N50656 | AW450348 | Hs.93996 | ESTs, Highly similar to SORL_HUMAN SORTI |
| 452259 | N50721 | AA317439 | Hs.28707 | signal sequence receptor, gamma (translo |
| 133057 | N53143 | AA465131 | Hs.64001 | Homo sapiens clone 25218 mRNA sequence |
| 118103 | N55326 | AA401733 | Hs.184134 | ESTs |
| 118111 | N55493 | N55493 | | gb: yv50c02 s1 Soares fetal liver spleen |
| 118129 | N57493 | N57493 | | gb: yy54c08.s1 Soares_multiple_sclerosis_{—} |
| 118278 | N62955 | N62955 | Hs.316433 | Homo sapiens cDNA FLJ11375 fis, clone HE |
| 118329 | N63520 | N63520 | | gb: yy62f01.s1 Soares_multiple_sclerosis_{—} |
| 118336 | N63604 | BE327311 | Hs.47166 | HT021 |
| 417098 | N64166 | AB017365 | Hs.173859 | frizzled (Drosophila) homolog 7 |
| 118363 | N64168 | AI183838 | Hs.48938 | hypothetical protein FLJ21802 |
| 118364 | N64191 | N46114 | Hs.29169 | hypothetical protein FLJ22623 |
| 118475 | N66845 | N66845 | | gb: za46c11 s1 Soares fetal liver spleen |
| 118491 | N67135 | AV647908 | Hs 90424 | Homo sapiens cDNA: FLJ23285 fis, clone H |
| 118500 | N67295 | W32889 | Hs.154329 | ESTs |
| 118584 | N68963 | AW136928 | | gb: Ul-H-Bl-adp-d-08-0-Ul.sl NCl_CGAP_Su |
| 456647 | N69331 | AI252640 | Hs 110364 | peptidylprolyl isomerase C (cyclophilin |
| 118661 | N70777 | AL137554 | Hs 49927 | protein kinase NYD-SP15 |
| 118684 | N71364 | N71313 | Hs.163986 | Homo sapiens cDNA: FLJ22765 fis, clone K |
| 118689 | N71545 | AW390601 | Hs.184544 | Homo sapiens, clone IMAGE:3355383, mRNA, |
| 118690 | N71571 | N71571 | Hs.269142 | ESTs |
| 118766 | N74456 | N74456 | Hs.50499 | EST |
| 118793 | N75594 | N75594 | Hs.285921 | ESTs, Moderately similar to T47135 hypot |
| 118817 | N79035 | AI668658 | Hs.50797 | ESTs |
| 118844 | N80279 | AL035364 | Hs.50891 | hypothetical protein |
| 118919 | N91797 | AW452696 | Hs.130760 | myosin phosphatase, target subunit 2 |
| 129558 | N92454 | AW580922 | Hs.180446 | karyophenn (importin) beta 1 |
| 407604 | N94581 | AW191962 | Hs 288061 | collagen, type VIII, alpha 2 |
| 118996 | N94746 | N94746 | Hs 274248 | hypothetical protein FLJ20758 |
| 119021 | N98238 | N98238 | Hs.55185 | ESTs |
| 119039 | R02384 | AI160570 | Hs.252097 | pregnancy specific beta-1-glycoprotein 6 |
| 119063 | R16833 | R16833 | Hs.53106 | ESTs, Moderately similar to ALU1_HUMAN A |
| 332622 | R41828 | R10674 | | CSR1 protein |
| 119111 | R43203 | T02865 | Hs.328321 | EST |
| 415115 | R46395 | AA214228 | Hs.127751 | hypothetical protein |
| 119146 | R58863 | R58863 | Hs.91815 | ESTs |
| 449224 | R78248 | AW995911 | Hs.299883 | hypothetical protein FLJ23399 |
| 119239 | T11483 | T11483 | | gb: CHR90049 Chromosome 9 exon Homo sapie |
| 119281 | T16896 | AI692322 | Hs 65373 | ESTs, Weakly similar to T02345 hypotheti |
| 119298 | T23820 | NM_001241 | Hs.155478 | cyclin T2 |
| 126502 | T30222 | T10077 | Hs.13453 | hypothetical protein FLJ14753 |
| 419983 | W15275 | W55956 | Hs 94030 | Homo sapiens mRNA; cDNA DKFZp586E1624 (f |
| 119558 | W38194 | W38194 | | Empirically selected from AFFX single pr |
| 429641 | W42414 | AW081883 | Hs.211578 | Homo sapiens cDNA: FLJ23037 fis, clone L |
| 419445 | W49632 | AA884471 | Hs.90449 | Human clone 23908 mRNA sequence |
| 119650 | W57613 | R82342 | Hs 79856 | ESTs, Weakly similar to S65657 alpha-1C- |
| 119654 | W57759 | W57759 | | gb: zd20g11.s1 Soares_fetal_heart_NbHH19W |
| 119683 | W61118 | W65379 | Hs.57835 | ESTs |
| 119694 | W65344 | AA041350 | Hs.57847 | ESTs, Moderately similar to ICE4_HUMAN C |
| 119718 | W69216 | W69216 | Hs.92848 | ESTs |
| 410365 | W69379 | AI287518 | | Homo sapiens mRNA; cDNA DKFZp586D0923 (f |
| 119938 | W86728 | AW014862 | Hs.58885 | ESTs |
| 120128 | Z38499 | BE379320 | Hs.91448 | MKP-1 like protein tyrosine phosphatase |
| 120130 | Z38630 | AA045767 | Hs.5300 | bladder cancer associated protein |
| 120148 | Z39494 | F02806 | Hs.65765 | ESTs |
| 120155 | Z39623 | Z39623 | Hs.65783 | ESTs |
| 451979 | Z40071 | F06972 | Hs.27372 | BMX non-receptor tyrosine kinase |
| 120183 | Z40174 | AW082866 | Hs 65882 | ESTs |
| 120184 | Z40182 | Z40182 | Hs.65885 | EST |
| 120211 | Z40904 | Z40904 | Hs.66012 | EST |
| 120245 | AA166965 | AW959615 | Hs.111045 | ESTs |
| 120247 | AA167500 | AA167500 | Hs.103939 | EST |
| 120254 | AA169599 | W90403 | Hs.111054 | ESTs |
| 120259 | AA171724 | AW014786 | Hs.192742 | hypothetical protein FLJ12785 |
| 120260 | AA171739 | AK000061 | Hs.101590 | hypothetical protein |
| 120275 | AA177105 | AA177105 | Hs.78457 | solute carrier family 25 (mitochondrial |
| 120284 | AA182626 | AA179656 | | gb: zp54e11.s1 Stratagene NT2 neuronal pr |
| 417735 | AA186324 | AA188175 | Hs.82506 | KIAA1254 protein |
| 422137 | AA192099 | AJ236885 | | zinc finger protein 148 (pHZ-52) |
| 120302 | AA192173 | AA837098 | Hs.269933 | ESTs |
| 120303 | AA192415 | AI216292 | Hs.96184 | ESTs |
| 120305 | AA192553 | AW295096 | Hs.101337 | uncoupling protein 3 (mitochondrial, pro |
| 120319 | AA194851 | T57776 | Hs 191094 | ESTs |
| 408729 | AA195520 | AA195764 | Hs.72639 | ESTs |
| 120326 | AA196300 | AA196300 | Hs.21145 | hypothetical protein RG083M05.2 |
| 133145 | AA196549 | H94227 | Hs.6592 | Homo sapiens, clone IMAGE:2961368, mRNA, |
| 120327 | AA196721 | AK000292 | Hs.130732 | hypothetical protein FLJ20285 |
| 120328 | AA196979 | AA923278 | Hs.290905 | ESTs, Weakly similar to protease [H. sapi |
| 120340 | AA206828 | AA206828 | | gb: zq80b08.s1 Stratagene hNT neuron (937 |
| 417122 | AA207123 | AI906291 | Hs.81234 | immunoglobulin superfamily, member 3 |
| 131522 | AA214539 | AI380040 | Hs.239489 | TIA1 cytotoxic granule-associated RNA-bi |
| 421787 | AA226914 | AA227068 | Hs.108301 | nuclear receptor subfamily 2, group C, m |
| 120375 | AA227260 | AF028706 | Hs 111227 | Zic family member 3 (odd-paired Drosophi |
| 120376 | AA227469 | AA227469 | | gb: zr18a07.s1 Stratagene NT2 neuronal pr |
| 120390 | AA233122 | AA837093 | Hs 111460 | calcium/calmodulin-dependent protein kin |
| 410804 | AA233334 | U64820 | Hs.66521 | Machado-Joseph disease (spinocerebellar |
| 434223 | AA233347 | AI825842 | Hs.3776 | zinc finger protein 216 |
| 312771 | AA233714 | AA018515 | Hs 264482 | Homo sapiens mRNA; cDNA DKFZp761A0411 (f |
| 120396 | AA233796 | AA134006 | Hs 79306 | eukaryotic translation initiation factor |
| 120409 | AA235050 | AA235050 | | gb: zs38e04.s1 Soares_NhHMPu_S1 Homo sapi |
| 120414 | AA235704 | AW137156 | Hs.181202 | hypothetical protein FLJ10038 |
| 120420 | AA236031 | AI128114 | Hs.112885 | spinal cord-derived growth factor-B |
| 120422 | AA236352 | AL133097 | Hs.301717 | hypothetical protein DKFZp434N1928 |
| 419326 | AA236390 | W94915 | Hs 42419 | ESTs |
| 120423 | AA236453 | AA236453 | Hs 18978 | Homo sapiens cDNA: FLJ22822 fis, clone K |
| 120435 | AA243370 | AA243370 | Hs.96450 | EST |
| 120453 | AA250947 | AA250947 | Hs.170263 | tumor protein p53-bindmg protein, 1 |
| 120455 | AA251083 | AA251720 | Hs.104347 | ESTs, Weakly similar to ALUC_HUMAN !!!! |
| 120456 | AA251113 | AA488750 | Hs.88414 | BTB and CNC homology 1, basic leucine zi |
| 120473 | AA251973 | AA251973 | Hs.269988 | ESTs |
| 128922 | AA252023 | AI244901 | Hs.9589 | ubiquilin 1 |
| 120477 | AA252414 | AA252414 | Hs.43141 | DKFZP727C091 protein |
| 120479 | AA252650 | AF006689 | Hs.110299 | mitogen-activated protein kinase kinase |
| 120488 | AA255523 | AW952916 | Hs.63510 | KIAA0141 gene product |
| 120510 | AA258128 | AI796395 | Hs.111377 | ESTs |
| 120527 | AA262105 | AA262105 | Hs.4094 | Homo sapiens cDNA FLJ14208 fis, clone NT |
| 120528 | AA262107 | AI923511 | Hs.104413 | ESTs |
| 120529 | AA262235 | AI434823 | Hs.104415 | ESTs |
| 120541 | AA278298 | W07318 | Hs.240 | M-phase phosphoprotein 1 |
| 120544 | AA278721 | BE548277 | Hs.103104 | ESTs |
| 120562 | AA280036 | BE244580 | Hs 342307 | hypothetical protein FLJ10330 |
| 120569 | AA280648 | AA807544 | Hs.24970 | ESTs, Weakly similar to B34323 GTP-bindi |
| 120571 | AA280738 | AB037744 | Hs.34892 | KIAA1323 protein |
| 120572 | AA280794 | H39599 | Hs 294008 | ESTs |
| 129434 | AA280837 | AW967495 | Hs.186644 | ESTs |
| 130529 | AA280886 | AA178953 | Hs 309648 | gb: zp39e03.s1 Stratagene muscle 937209 H |
| 120575 | AA280934 | AW978022 | Hs.238911 | hypothetical protein DKFZp762E1511; KIAA |
| 409339 | AA281535 | AB020686 | Hs.54037 | ectonucleotide pyrophosphatase/phosphodi |
| 120591 | AA281797 | AF078847 | Hs.191356 | general transcription factor IIH, polype |
| 120593 | AA282047 | AA748355 | Hs.193522 | ESTs |
| 430275 | AA283002 | Z11773 | Hs.237786 | zinc finger protein 187 |
| 440303 | AA283709 | AA306166 | Hs 7145 | calpain 7 |
| 120609 | AA283902 | AW978721 | Hs.266076 | ESTs, Weakly similar to A46010 X-linked |
| 409702 | AA284108 | AI752244 | | eukaryotic translation elongation factor |
| 456870 | AA284109 | AI241084 | Hs.154353 | nonselective sodium potassium/proton exc |
| 132614 | AA284371 | AA284371 | Hs.118064 | similar to rat nuclear ubiquitous casein |
| 458750 | AA284744 | AA115496 | Hs.336898 | Homo sapiens, Similar to RIKEN cDNA 1810 |
| 135376 | AA284784 | BE617856 | Hs.99756 | mitochondrial ribosome recycling factor |
| 120621 | AA284840 | AW961294 | Hs.143818 | hypothetical protein FLJ23459 |
| 452279 | AA286844 | AA286844 | Hs 61260 | hypothetical protein FLJ13164 |
| 332484 | AA287032 | AW172431 | Hs.13012 | ESTs |
| 120644 | AA287038 | AI869129 | Hs.96616 | ESTs |
| 120660 | AA287546 | AA286785 | Hs.99677 | ESTs |
| 135370 | AA287553 | BE622187 | Hs.99670 | ESTs, Weakly similar to I38022 hypotheti |
| 120661 | AA287556 | AA287556 | Hs.263412 | ESTs, Weakly similar to ALUB_HUMAN !!!! |
| 429828 | AA287564 | AB019494 | Hs.225767 | IDN3 protein |
| 452291 | AA291015 | AF015592 | Hs 28853 | CDC7 (cell division cycle 7, S. cerevisi |
| 120699 | AA291716 | AI683243 | Hs.97258 | ESTs, Moderately similar to S29539 ribos |
| 100690 | AA291749 | AA383256 | Hs.1657 | estrogen receptor 1 |
| 120726 | AA293656 | AA293655 | Hs.21198 | ESTs |
| 120737 | AA302430 | AL049176 | Hs.82223 | chordin-like |
| 120745 | AA302809 | AA302809 | | gb: EST10426 Adipose tissue, white I Homo |
| 443574 | AA302820 | U83993 | Hs.321709 | purinergic receptor P2X, ligand-gated 10 |
| 120750 | AA310499 | AI191410 | Hs 96693 | ESTs, Moderately similar to 2109260A B c |
| 120761 | AA321890 | AA321890 | | branched chain keto acid dehydrogenase E |
| 120768 | AA340589 | AA340589 | Hs 104560 | EST |
| 120769 | AA340622 | AI769467 | Hs.9475 | ESTs |
| 135232 | AA342457 | AL038812 | Hs.96800 | ESTs, Moderately similar to ALU7_HUMAN A |
| 120793 | AA342864 | AA342864 | Hs 96812 | ESTs |
| 120796 | AA342973 | AI247356 | Hs.96820 | ESTs |
| 120809 | AA346495 | AA346495 | | gb: EST52657 Fetal heart II Homo sapiens |
| 332633 | AA347573 | AL120071 | Hs.48998 | fibronectin leucine rich transmembrane p |
| 120825 | AA347614 | AI280215 | Hs.96885 | ESTs |
| 120827 | AA347717 | AA382525 | Hs.132967 | Human EST clone 122887 mariner transposo |
| 120839 | AA348913 | AA348913 | | gb: EST55442 Infant adrenal gland II Homo |
| 120850 | AA349647 | AA349647 | Hs.96927 | Homo sapiens cDNA FLJ12573 fis, clone NT |
| 120852 | AA349773 | AA349773 | Hs 191564 | ESTs |
| 128852 | AA350541 | R40622 | Hs.106601 | ESTs |
| 135240 | AA357159 | AA357159 | Hs.96986 | EST |
| 120870 | AA357172 | AA357172 | Hs.292581 | ESTs, Moderately similar to ALU1_HUMAN A |
| 120894 | AA370132 | AA370132 | Hs.97063 | ESTs |
| 435737 | AA370472 | AF229839 | Hs.173202 | I-kappa-B-interacting Ras-like protein 1 |
| 120897 | AA370867 | AA370867 | Hs 97079 | ESTs, Moderately similar to AF174605 1 F |
| 120915 | AA377296 | AL135556 | Hs.97104 | ESTs |
| 120935 | AA383902 | AL048409 | Hs.97177 | ESTs, Weakly similar to ALU1_HUMAN ALU S |
| 120936 | AA385934 | AA385934 | Hs 97184 | EST, Highly similar to (defline not aval |
| 120937 | AA386255 | AA386255 | Hs.97186 | EST |
| 120938 | AA386260 | AA386260 | Hs.104632 | EST |
| 417632 | AA386266 | R20855 | Hs.5422 | glycoprotein M6B |
| 120960 | AA398014 | AA398014 | Hs.104684 | EST |
| 120985 | AA398222 | AI219896 | Hs.97592 | ESTs |
| 120988 | AA398235 | AA398235 | Hs 97631 | ESTs |
| 121008 | AA398348 | AA398348 | Hs.130546 | Human DNA sequence from clone RP11-251J8 |
| 121029 | AA398482 | AA398482 | Hs 97641 | EST |
| 121032 | AA398504 | AA393037 | Hs.161798 | ESTs |
| 121033 | AA398505 | AA398505 | Hs.97360 | ESTs |
| 121034 | AA398507 | AL389951 | Hs 271623 | nucleoporin 50 kD |
| 121035 | AA398523 | AA398523 | Hs 210579 | ESTs |
| 121058 | AA398625 | AA398625 | Hs.97391 | ESTs |
| 121060 | AA398632 | AA398632 | Hs 97395 | ESTs |
| 121061 | AA398633 | AA393288 | Hs.97396 | ESTs |
| 121091 | AA398894 | AA398894 | Hs 97657 | ESTs, Moderately similar to ALU8_HUMAN A |
| 121092 | AA398895 | AA398895 | Hs.97658 | EST |
| 121094 | AA398900 | AA402505 | | gb: zt62h10.r1 Soares_testis_NHT Homo sap |
| 121096 | AA398904 | AA398904 | Hs.332690 | ESTs |
| 121115 | AA399122 | AA398187 | Hs.104682 | ESTs, Weakly similar to mitochondrial ci |
| 121121 | AA399371 | AA399371 | Hs.189095 | similar to SALL1 (sal (Drosophila)-like |
| 121122 | AA399373 | AI126713 | Hs.192233 | ESTs; Highly similar to T00337 hypotheti |
| 121125 | AA399441 | AL042981 | Hs.251278 | KIAA1201 protein |
| 121151 | AA399636 | AA399636 | Hs.143629 | ESTs |
| 121153 | AA399640 | AA399640 | Hs.97694 | ESTs |
| 121163 | AA399680 | AI676062 | Hs.111902 | ESTs |
| 121176 | AA400080 | AL121523 | Hs.97774 | ESTs |
| 121192 | AA400262 | AA400262 | Hs.190093 | ESTs |
| 121223 | AA400725 | AI002110 | Hs.97169 | ESTs, Weakly similar to dJ667H12.2.1 [H. |
| 121227 | AA400748 | AA400748 | Hs.97823 | Homo sapiens mRNA; cDNA DKFZp434D024 (fr |
| 121231 | AA400780 | AA814948 | Hs.96343 | ESTs, Weakly similar to ALUC_HUMAN !!!! |
| 121278 | AA401631 | AA037121 | Hs.98518 | Homo sapiens cDNA FLJ11490 fis, clone HE |
| 121279 | AA401688 | AA292873 | Hs 177996 | ESTs |
| 121282 | AA401695 | AA401695 | Hs 97334 | ESTs |
| 121299 | AA402227 | AA402227 | Hs.22826 | tropomodulin 3 (ubiquitous) |
| 121301 | AA402329 | NM_006202 | Hs.89901 | phosphodiesterase 4A, cAMP-specific (dun |
| 121302 | AA402398 | AA402587 | Hs 325520 | LAT1-3TM protein |
| 121304 | AA402449 | AA293863 | Hs.97316 | EST |
| 121305 | AA402468 | AA402468 | Hs 291557 | ESTs |
| 134721 | AA403268 | AK000112 | Hs.89306 | hypothetical protein FLJ20105 |
| 121323 | AA403314 | AA291411 | Hs.97247 | ESTs |
| 121324 | AA404229 | AA404229 | Hs.97842 | EST |
| 444422 | AA404260 | AI768623 | Hs.108264 | ESTs |
| 131074 | AA404271 | U16125 | Hs.181581 | glutamate receptor, ionotropic, kainate |
| 121344 | AA405026 | AA405026 | Hs.193754 | ESTs |
| 121348 | AA405182 | AA405182 | Hs.97973 | ESTs |
| 121350 | AA405237 | AA405237 | | gb: zt06e10.s1 NCl_CGAP_GCB1 Homo sapiens |
| 121400 | AA406061 | AA406061 | Hs.98001 | EST |
| 121402 | AA406063 | AA406063 | Hs 98003 | ESTs |
| 121403 | AA406070 | AA406070 | Hs 98004 | EST |
| 121408 | AA406137 | AA406137 | Hs.98019 | EST |
| 121431 | AA406335 | AA035279 | Hs.176731 | ESTs |
| 121471 | AA411804 | AA411804 | Hs.261575 | ESTs |
| 121474 | AA411833 | AA402335 | Hs.188760 | ESTs, Highly similar to Trad [H. sapiens] |
| 121526 | AA412219 | AW665325 | Hs.98120 | ESTs |
| 121530 | AA412259 | AA778658 | Hs.98122 | ESTs |
| 121558 | AA412497 | AA412497 | | gb: zt95g12.s1 Soares_testis_NHT Homo sap |
| 121559 | AA412498 | AI192044 | Hs.104778 | ESTs |
| 121584 | AA416586 | AI024471 | Hs.98232 | ESTs |
| 121609 | AA416867 | AA416867 | Hs.98185 | EST |
| 121612 | AA416874 | AA416874 | Hs.98168 | ESTs |
| 121737 | AA421133 | AA421133 | Hs 104671 | erythrocyte transmembrane protein |
| 121740 | AA421138 | AA421138 | Hs 143835 | EST |
| 436032 | AA422079 | AA150797 | Hs.109276 | latexin protein |
| 121784 | AA423837 | T90789 | Hs.94308 | RAB35, member RAS oncogene family |
| 121802 | AA424328 | AI251870 | Hs.188898 | ESTs |
| 121803 | AA424339 | AI338371 | Hs 157173 | ESTs |
| 135286 | AA424469 | AW023482 | Hs.97849 | ESTs |
| 332778 | AA424469 | AW023482 | Hs.97849 | ESTs |
| 121806 | AA424502 | AA424313 | Hs.98402 | ESTs |
| 129517 | AA425004 | AW972853 | Hs.112237 | ESTs |
| 121845 | AA425734 | AI732692 | Hs 165066 | ESTs, Moderately similar to ALU2_HUMAN A |
| 121853 | AA425887 | AA425887 | Hs.98502 | hypothetical protein FLJ14303 |
| 121891 | AA426456 | AA426456 | Hs.98469 | ESTs |
| 121895 | AA427396 | AA427396 | | gb: zw33a02.s1 Soares ovary tumor NbHOT H |
| 121899 | AA427555 | R55341 | Hs.50421 | KIAA0203 gene product |
| 121917 | AA428218 | AA406397 | Hs 139425 | ESTs |
| 121918 | AA428242 | BE274689 | Hs.184175 | chromosome 2 open reading frame 3 |
| 121919 | AA428281 | AA428281 | Hs.98560 | EST |
| 121941 | AA428865 | AA428865 | Hs 98563 | ESTs |
| 121942 | AA428994 | AW452701 | Hs.293237 | ESTs |
| 121970 | AA429666 | AA429666 | Hs.98617 | EST |
| 121993 | AA430181 | AW297880 | Hs 98661 | ESTs |
| 418706 | AA430184 | U73524 | Hs.87465 | ATP/GTP-binding protein |
| 122022 | AA431293 | AA431293 | Hs.98716 | ESTs, Moderately similar to T42650 hypot |
| 122050 | AA431478 | AI453076 | | ELAV (embryonic lethal, abnormal vision, |
| 122051 | AA431492 | AA431492 | Hs 98742 | EST |
| 122055 | AA431732 | AA431732 | Hs.98747 | EST |
| 122105 | AA432278 | AW241685 | Hs.98699 | ESTs |
| 122125 | AA434411 | AK000492 | Hs.98806 | hypothetical protein |
| 135235 | AA435512 | AW298244 | Hs 266195 | ESTs |
| 122162 | AA435698 | AA628233 | Hs.79946 | cytochrome P450, subfamily XIX (aromatiz |
| 422072 | AA435711 | AB018255 | Hs.111138 | KIAA0712 gene product |
| 415106 | AA435815 | U40763 | Hs 77965 | peptidyl-prolyl isomerase G (cyclophilin |
| 122186 | AA435842 | AA398811 | Hs.104673 | ESTs |
| 122235 | AA436475 | AA436475 | Hs.112227 | membrane-associated nucleic acid binding |
| 412970 | AA436489 | AB026436 | Hs 177534 | dual specificity phosphatase 10 |
| 419288 | AA442060 | AA256106 | Hs.87507 | ESTs |
| 122310 | AA442079 | AW192803 | Hs.98974 | ESTs, Weakly similar to S65824 reverse t |
| 122334 | AA443151 | BE465894 | Hs.98365 | ESTs, Weakly similar to LB4D HUMAN NADP- |
| 122382 | AA446133 | AA446440 | Hs 98643 | ESTs |
| 122425 | AA447145 | AB007859 | Hs.100955 | KIAA0399 protein |
| 122431 | AA447398 | AA447398 | Hs.99104 | ESTs |
| 122450 | AA447643 | AA447643 | Hs.112095 | hypothetical protein DKFZp434F1819 |
| 426284 | AA447742 | AJ404468 | Hs.284259 | dynein, axonemal, heavy polypeptide 9 |
| 122477 | AA448226 | AA448226 | Hs.324123 | ESTs |
| 122500 | AA448825 | AA448825 | Hs 99190 | ESTs |
| 122522 | AA449444 | AA299607 | Hs.98969 | ESTs |
| 122536 | AA450087 | AF060877 | Hs.99236 | regulator of G-protein signalling 20 |
| 122538 | AA450211 | AA450211 | Hs.99239 | ESTs |
| 122540 | AA450244 | AA476741 | Hs.98279 | ESTs, Weakly similar to A43932 mucin 2 p |
| 122560 | AA452123 | AW392342 | Hs.283077 | centrosomal P4.1 -associated protein; unc |
| 421919 | AA452155 | AJ224901 | Hs 109526 | zinc finger protein 198 |
| 122562 | AA452156 | AA452156 | | gb: zx29c03 s1 Soares_total_fetus_Nb2HF8_{—} |
| 122585 | AA453036 | AI681654 | Hs.170737 | hypothetical protein FLJ23251 |
| 122608 | AA453526 | AA453525 | Hs 143077 | ESTs |
| 122635 | AA454085 | AA454085 | | gb: zx33a08.s1 Soares_total_fetus_Nb2HF8_{—} |
| 122636 | AA454103 | AW651706 | Hs.99519 | hypothetical protein FLJ14007 |
| 122653 | AA454642 | AW009166 | Hs.99376 | ESTs |
| 122660 | AA454935 | AI816827 | Hs.180069 | nuclear respiratory factor 1 |
| 122703 | AA456323 | AA456323 | Hs.269369 | ESTs |
| 122724 | AA457395 | AA457395 | Hs.99457 | ESTs |
| 122749 | AA458850 | AA458850 | Hs 293372 | ESTs, Weakly similar to B34087 hypotheti |
| 122772 | AA459662 | AW117452 | Hs.99489 | ESTs |
| 430242 | AA459668 | U66669 | Hs.236642 | 3-hydroxyisobutyryl-Coenzyme A hydrolase |
| 429838 | AA459679 | AW904907 | Hs.30732 | hypothetical protein FLJ13409; KIAA1711 |
| 122777 | AA459702 | AK001022 | Hs 214397 | hypothetical protein FLJ10160 similar to |
| 135362 | AA460017 | AA978128 | Hs 99513 | ESTs, Weakly similar to T17454 diaphanou |
| 122798 | AA460324 | AW366286 | Hs.145696 | splicing factor (CC1. 3) |
| 122837 | AA461509 | AA461509 | Hs.293565 | ESTs, Weakly similar to putative p150 [H |
| 122860 | AA464414 | AA464414 | | gb: zx78g01 s1 Soares ovary tumor NbHOT H |
| 122861 | AA464428 | AA335721 | Hs 213628 | ESTs |
| 122910 | AA470084 | AA470084 | Hs.98358 | ESTs |
| 132899 | AA476606 | AA476606 | Hs.59666 | SMAD in the antisense orientation |
| 122967 | AA478521 | AA806187 | Hs.289101 | glucose regulated protein, 58 kD |
| 422845 | AA478523 | AA317841 | Hs.7845 | hypothetical protein MGC2752 |
| 123009 | AA479949 | AA535244 | Hs.78305 | RAB2, member RAS oncogene family |
| 128917 | AA481252 | AI365215 | Hs.206097 | oncogene TC21 |
| 123081 | AA485351 | AI815486 | Hs.243901 | Homo sapiens cDNA FLJ20738 fis, clone HE |
| 123133 | AA487264 | AA487264 | Hs.154974 | Homo sapiens mRNA; cDNA DKFZp667N064 (fr |
| 123184 | AA489072 | BE247767 | Hs.18166 | KIAA0870 protein |
| 332467 | AA489630 | NM_014700 | Hs.119004 | KIAA0665 gene product |
| 123233 | AA490225 | AW974175 | Hs.151875 | ESTs, Weakly similar to MAPB_HUMAN MICRO |
| 123234 | AA490227 | NM_001938 | Hs.16697 | down-regulator of transcription 1, TBP-b |
| 123236 | AA490255 | AW968504 | Hs.123073 | CDC2-related protein kinase 7 |
| 123255 | AA490890 | AA830335 | Hs.105273 | ESTs |
| 430015 | AA490916 | AW768399 | Hs.106357 | ESTs |
| 448892 | AA490925 | AF084535 | Hs 22464 | epilepsy, progressive myoclonus type 2, |
| 123259 | AA490955 | AI744152 | Hs.283374 | ESTs, Weakly similar to CA15_HUMAN COLLA |
| 123284 | AA495812 | AA488988 | Hs 293796 | ESTs |
| 123286 | AA495824 | AA495824 | Hs 188822 | ESTs, Weakly similar to A46010 X-linked |
| 123315 | AA496369 | AA496369 | | gb: zv37d10.s1 Soares ovary tumor NbHOT H |
| 457397 | AA504125 | AW969025 | Hs 109154 | ESTs |
| 433049 | AA521473 | AU076668 | Hs.334884 | SEC10 (S. cerevisiae)-like 1 |
| 123421 | AA598440 | AA598440 | Hs 291154 | EST, Weakly similar to I38022 hypothetic |
| 123449 | AA598899 | AL049325 | Hs.112493 | Homo sapiens mRNA, cDNA DKFZp564D036 (fr |
| 426981 | AA599244 | AL044675 | Hs.173081 | KIAA0530 protein |
| 409986 | AA599694 | NM_014777 | Hs.57730 | KIAA0133 gene product |
| 123497 | AA600037 | AA765256 | Hs.135191 | ESTs, Weakly similar to unnamed protein |
| 123604 | AA609135 | AA609135 | Hs 293076 | ESTs |
| 123712 | AA609684 | AA609684 | | Homo sapiens cDNA: FLJ21543 fis, clone C |
| 123731 | AA609839 | AA609839 | Hs.334437 | gb: ae62f01.s1 Stratagene lung carcinoma |
| 123800 | AA620423 | AA620423 | Hs.112862 | EST |
| 123841 | AA620747 | AA620747 | Hs 112896 | ESTs |
| 123929 | AA621364 | AA621364 | Hs.112981 | ESTs |
| 123978 | C20653 | T89832 | Hs.170278 | ESTs |
| 133184 | D20085 | AA001021 | Hs.6685 | thyroid hormone receptor interactor 8 |
| 132835 | D20749 | Z83844 | Hs.5790 | hypothetical protein dJ37E16.5 |
| 435147 | D51285 | AL133731 | Hs.4774 | Homo sapiens mRNA, cDNA DKFZp761C1712 (f |
| 128695 | D59972 | NM_003478 | Hs.101299 | cullin 5 |
| 124029 | F04112 | F04112 | Hs.312553 | gb: HSC2JH062 normalized infant brain cDN |
| 124057 | F13604 | AA902384 | Hs.73853 | bone morphogenetic protein 2 |
| 449316 | H01662 | AI609045 | Hs.321775 | hypothetical protein DKFZp434D1428 |
| 130973 | H05135 | AI638418 | Hs.1440 | DEAD/H (Asp-Glu-Ala-Asp/His) box polypep |
| 124106 | H12245 | H12245 | | gb: ym17a12.r1 Soares infant brain 1NIB H |
| 124136 | H22842 | H22842 | Hs.101770 | EST |
| 124165 | H30894 | H30039 | Hs 107674 | ESTs |
| 429627 | H43442 | NM_015340 | Hs.2450 | leucyl-tRNA synthetase, mitochondrial |
| 124178 | H45996 | BE463721 | Hs.97101 | putative G protein-coupled receptor |
| 129948 | H69281 | AI537162 | Hs 263988 | ESTs |
| 452114 | H69485 | N22687 | Hs 8236 | ESTs |
| 124 + D826254 | | H69899 | H69899 | gb: yu70c12 s1 Weizmann Olfactory Epithel |
| 129056 | H70627 | AI769958 | Hs 108336 | ESTs, Weakly similar to ALUE_HUMAN !!!! |
| 427580 | H73260 | AK001507 | Hs.44143 | Homo sapiens clone FLB6914 PRO1821 mRNA, |
| 426793 | H77531 | X89887 | Hs.172350 | HIR (histone cell cycle regulation defec |
| 124274 | H80552 | H80552 | Hs.102249 | EST |
| 129078 | H80737 | AI351010 | Hs.102267 | lysosomal |
| 457658 | H93412 | AW952124 | Hs.13094 | presenilins associated rhomboid-like pro |
| 124315 | H94892 | NM_005402 | Hs.288757 | v-ral simian leukemia viral oncogene horn |
| 437712 | H95643 | X04588 | Hs.85844 | neurotrophic tyrosine kinase, receptor, |
| 124324 | H96552 | H96552 | Hs.159472 | Homo sapiens cDNA: FLJ22224 fis, clone H |
| 452933 | H97146 | AW391423 | Hs.288555 | Homo sapiens cDNA: FLJ22425 fis, clone H |
| 132231 | H99131 | AA662910 | Hs.42635 | hypothetical protein DKFZp434K2435 |
| 421877 | H99462 | AW250380 | Hs.109059 | mitochondrial ribosomal protein L12 |
| 443123 | H99837 | AA094538 | Hs.272808 | putative transcription regulation nuclea |
| 132963 | N22140 | AA099693 | Hs 34851 | epsilon-tubulin |
| 420473 | N22197 | AL118782 | Hs.300208 | Sec23-interactmg protein p125 |
| 417381 | N23756 | AF164142 | Hs.82042 | solute carrier family 23 (nucleobase tra |
| 130365 | N24134 | W56119 | Hs.155103 | eukaryotic translation initiation factor |
| 456610 | N24195 | AF172066 | Hs.106346 | retinoic acid repressible protein |
| 439311 | N26739 | BE270668 | Hs.151945 | mitochondrial ribosomal protein L43 |
| 124383 | N27098 | N27098 | Hs.102463 | EST |
| 124387 | N27637 | N27637 | Hs.109019 | ESTs |
| 129341 | N33090 | AI193519 | Hs 226396 | hypothetical protein FLJ11126 |
| 419793 | N35967 | AI364933 | Hs.168913 | serine/threonine kinase 24 (Ste20, yeast |
| 124433 | N39069 | AA280319 | Hs 288840 | PRO1575 protein |
| 124441 | N46441 | AW450481 | Hs.161333 | ESTs |
| 132338 | N48270 | AA353868 | Hs 182982 | golgin-67 |
| 436575 | N48365 | AI473114 | | ESTs |
| 124466 | N51316 | R10084 | Hs.113319 | kinesin heavy chain member 2 |
| 408048 | N51499 | NM_007203 | Hs 42322 | A kinase (PRKA) anchor protein 2 |
| 124483 | N53976 | AI821780 | Hs.179864 | ESTs |
| 124484 | N54157 | H66118 | Hs.285520 | ESTs, Weakly similar to 2109260A B cell |
| 124485 | N54300 | AB040933 | Hs.15420 | KIAA1500 protein |
| 124494 | N54831 | N54831 | Hs 271381 | ESTs, Weakly similar to I38022 hypotheti |
| 129200 | N59849 | N59849 | Hs.13565 | Sam68-like phosphotyrosine protein, T-ST |
| 124527 | N62132 | N79264 | Hs.269104 | ESTs |
| 124532 | N62375 | N62375 | Hs.102731 | EST |
| 133213 | N63138 | AA903424 | Hs.6786 | ESTs |
| 124539 | N63172 | D54120 | Hs.146409 | cell division cycle 42 (GTP-binding prot |
| 129196 | N63787 | BE296313 | Hs.265592 | ESTs, Weakly similar to I38022 hypotheti |
| 124575 | N68168 | N68168 | | gb: za11c01 s1 Soares fetal liver spleen |
| 124576 | N68201 | N68201 | | ESTs, Weakly similar to I38022 hypotheti |
| 124577 | N68300 | N68300 | Hs.138485 | gb: za12g07 s1 Soares fetal liver spleen |
| 124578 | N68321 | N68321 | Hs.231500 | EST |
| 124593 | N69575 | N69575 | Hs.102788 | ESTs |
| 128501 | N75007 | AL133572 | Hs.199009 | protein containing CXXC domain 2 |
| 332434 | N75542 | AI680737 | Hs 289068 | Homo sapiens cDNA FLJ11918 fis; clone HE |
| 128473 | N90066 | T78277 | Hs.100293 | O-linked N-acetylglucosamine (GlcNAc) tr |
| 128639 | N91246 | AW582962 | Hs 102897 | CGI-47 protein |
| 124652 | N92751 | W19407 | Hs 3862 | regulator of nonsense transcripts 2; DKF |
| 133137 | N93214 | AB002316 | Hs.65746 | KIAA0318 protein |
| 124671 | N99148 | AK001357 | Hs.102951 | Homo sapiens cDNA FLJ10495 fis, clone NT |
| 133054 | R07876 | AA464836 | Hs.291079 | ESTs, Weakly similar to T27173 hypotheti |
| 425266 | R10865 | J00077 | Hs.155421 | alpha-fetoprotein |
| 124720 | R11056 | R05283 | | gb: ye91c08.s1 Soares fetal liver spleen |
| 124722 | R11488 | T97733 | Hs 185685 | ESTs |
| 128944 | R23930 | AL137586 | Hs.52763 | anaphase-promoting complex subunit 7 |
| 132965 | R26589 | AI248173 | Hs.191460 | hypothetical protein MGC12936 |
| 426504 | R37588 | AW162919 | Hs.170160 | RAB2, member RAS oncogene family-like |
| 438828 | R37613 | AL134275 | Hs 6434 | hypothetical protein DKFZp761F2014 |
| 124757 | R38398 | H11368 | Hs.141055 | Homo sapiens clone 23758 mRNA sequence |
| 124762 | R39179 | AA553722 | Hs.92096 | ESTs, Moderately similar to A46010 X-lin |
| 124773 | R40923 | R45154 | Hs.338439 | ESTs |
| 135266 | R41179 | R41179 | Hs 97393 | KIAA0328 protein |
| 427961 | R41294 | AW293165 | Hs.143134 | ESTs |
| 414303 | R42307 | NM_004427 | Hs.165263 | early development regulator 2 (homolog o |
| 128540 | R43189 | AW297929 | Hs.328317 | EST |
| 124785 | R43306 | W38537 | Hs.280740 | hypothetical protein MGC3040 |
| 124792 | R44357 | R44357 | Hs.48712 | hypothetical protein FLJ20736 |
| 124793 | R44519 | R44519 | | gb: yg24h04.s1 Soares infant brain 1NIB H |
| 124799 | R45088 | R45088 | | gb: yg38g04.s1 Soares infant brain 1NIB H |
| 124812 | R47948 | R47948 | Hs 188732 | ESTs |
| 124821 | R51524 | H87832 | Hs.7388 | kelch (Drosophila)-like 3 |
| 424123 | R54950 | AW966158 | Hs.58582 | Homo sapiens cDNA FLJ12789 fis, clone NT |
| 124835 | R55241 | R55241 | Hs.101214 | EST |
| 124845 | R59585 | R59585 | Hs.101255 | ESTs |
| 124847 | R60044 | W07701 | Hs.304177 | Homo sapiens clone FLB8503 PR02286 mRNA, |
| 440630 | R60872 | BE561430 | Hs.239388 | Human DNA sequence from clone RP1-304B14 |
| 124861 | R66690 | R67567 | Hs.107110 | ESTs |
| 332503 | R67266 | NM_004455 | Hs.150956 | exostoses (multiple)-like 1 |
| 124879 | R73588 | R73588 | Hs.101533 | ESTs |
| 124892 | R79403 | AI970003 | Hs.23756 | hypothetical protein similar to swine ac |
| 124906 | R87647 | H75964 | Hs 107815 | ESTs |
| 124922 | R93622 | R93622 | Hs.12163 | eukaryotic translation initiation factor |
| 124940 | R99599 | AF068846 | Hs.103804 | heterogeneous nuclear ribonucleoprotein |
| 124941 | R99612 | AI766661 | Hs 27774 | ESTs, Highly similar to AF161349 1 HSPC0 |
| 124943 | T02888 | AW963279 | Hs.123373 | ESTs, Weakly similar to ALU1_HUMAN ALU S |
| 124947 | T03170 | T03170 | Hs 100165 | ESTs |
| 124954 | T10465 | AW964237 | Hs.6728 | KIAA1548 protein |
| 456862 | T15418 | U55184 | Hs.154145 | hypothetical protein FLJ11585 |
| 410653 | T15597 | BE383768 | Hs.65238 | 95 kDa retinoblastoma protein binding pr |
| 418133 | T15652 | R43504 | Hs.6181 | ESTs |
| 440014 | T16898 | AW960782 | Hs 6856 | ash2 (absent, small, or homeotic, Drosop |
| 131082 | T26644 | AI091121 | Hs.246218 | Homo sapiens cDNA: FLJ21781 fis, clone H |
| 124980 | T40841 | T40841 | Hs.98681 | ESTs |
| 124984 | T47566 | BE313210 | Hs.334798 | eukaryotic translation elongation factor |
| 124991 | T50116 | T50116 | | gb: yb77c10 s1 Stratagene ovary (937217) |
| 457222 | T50145 | NM_004477 | Hs.203772 | FSHD region gene 1 |
| 125000 | T58615 | T58615 | Hs.235887 | ESTs |
| 132932 | T59940 | AW118826 | Hs 6093 | Homo sapiens cDNA: FLJ22783 fis, clone K |
| 444484 | T63595 | AK002126 | Hs.11260 | hypothetical protein FLJ11264 |
| 125008 | T64891 | T91251 | | gb: yd60a10.s1 Soares fetal liver spleen |
| 125009 | T64924 | T64924 | Hs.303046 | ESTs |
| 445384 | T64933 | T79136 | Hs.127243 | Homo sapiens mRNA for KIAA1724 protein, |
| 125017 | T68875 | T68875 | | gb: yc30f05.s1 Stratagene liver (937224) |
| 125018 | T69027 | T69027 | Hs.269481 | sex comb on midleg homolog 1 |
| 125020 | T69924 | T69981 | | gb: yc19d03.r1 Stratagene lung (937210) H |
| 437871 | T70353 | AI084813 | Hs.114088 | ESTs |
| 134204 | T79780 | AI873257 | Hs.7994 | hypothetical protein FLJ20551 |
| 125050 | T79951 | AW970209 | Hs.111805 | ESTs |
| 125052 | T80174 | T85104 | Hs.222779 | ESTs, Moderately similar to similar to N |
| 125054 | T80622 | T80622 | Hs.268601 | ESTs, Weakly similar to envelope [H. sapi |
| 125063 | T85352 | T85352 | | gb: yd82d01.s1 Soares fetal liver spleen |
| 125064 | T85373 | T85373 | | gb: yd82f07.s1 Soares fetal liver spleen |
| 125066 | T86284 | T86284 | | gb: yd77b07.s1 Soares fetal liver spleen |
| 416507 | T89579 | AL045364 | Hs.79353 | transcription factor Dp-1 |
| 125080 | T90360 | T90360 | Hs.268620 | ESTs, Highly similar to ALU6_HUMAN ALU S |
| 125097 | T94328 | AW576389 | Hs.335774 | EST, Moderately similar to S65657 alpha- |
| 125104 | T95590 | T95590 | | gb: ye40a03.s1 Soares fetal liver spleen |
| 135107 | T97257 | T97257 | Hs.94560 | ESTs, Moderately similar to I38022 hypot |
| 423122 | T97599 | AA845462 | Hs 124024 | deltex (Drosophila) homolog 1 |
| 125118 | T97620 | R10606 | Hs.269890 | gb: yf35f11.s1 Soares fetal liver spleen |
| 125120 | T97775 | T97775 | Hs.100717 | EST |
| 134160 | T98152 | T98152 | Hs 79432 | fibrillin 2 (congenital contractural ara |
| 125136 | W31479 | AW962364 | Hs.129051 | ESTs |
| 125144 | W37999 | AB037742 | Hs.24336 | KIAA1321 protein |
| 125150 | W38240 | W38240 | | Empirically selected from AFFX single pr |
| 450142 | W40150 | AW207469 | Hs 24485 | chondroitin sulfate proteoglycan 6 (bama |
| 131987 | W45435 | AW453069 | Hs 3657 | activity-dependent neuroprotective prote |
| 125178 | W58202 | W93127 | Hs.31845 | ESTs |
| 125180 | W58344 | W58469 | Hs.103120 | ESTs |
| 125182 | W58650 | AA451755 | Hs.263560 | ESTs |
| 446888 | W68736 | AL030996 | Hs 16411 | hypothetical protein LOC57187 |
| 125197 | W69106 | AF086270 | Hs.278554 | heterochromatin-like protein 1 |
| 133497 | W69111 | BE617303 | Hs 74266 | hypothetical protein MGC4251 |
| 429922 | W69399 | Z97630 | Hs 226117 | H1 histone family, member 0 |
| 129232 | W69459 | R98881 | Hs 109655 | sex comb on midleg (Drosophila)-like 1 |
| 422166 | W72424 | W72424 | Hs.112405 | S100 calcium-binding protein A9 (calgran |
| 125209 | W72724 | W72724 | Hs.103174 | ESTs, Weakly similar to TSP2_HUMAN THROM |
| 125212 | W72834 | AA746225 | Hs 103173 | ESTs |
| 456631 | W73955 | BE383436 | Hs.108847 | hypothetical protein MGC2749 |
| 125223 | W74701 | AI916269 | Hs.109057 | ESTs, Weakly similar to ALU5_HUMAN ALU S |
| 125225 | W76540 | W74169 | Hs.16492 | DKFZP564G2022 protein |
| 125228 | W79397 | AA033982 | Hs.110059 | ESTs, Weakly similar to I38022 hypotheti |
| 132393 | W85888 | AL135094 | Hs.47334 | hypothetical protein FLJ14495 |
| 125238 | W86038 | N99713 | Hs.109514 | ESTs |
| 125247 | W86881 | AA694191 | Hs.163914 | ESTs |
| 129296 | W87804 | AI051967 | Hs.110122 | ESTs |
| 125263 | W88942 | AA098878 | | gb: zn45g10.r1 Stratagene HeLa cell s3 93 |
| 125266 | W90022 | W90022 | Hs.186809 | ESTs, Highly similar to LCT2_HUMAN LEUKO |
| 450862 | W92272 | U91543 | Hs 25601 | chromodomain helicase DMA binding protei |
| 452401 | W92764 | NM_007115 | Hs.29352 | tumor necrosis factor, alpha-induced pro |
| 428243 | W93040 | H05317 | Hs.283549 | ESTs |
| 125277 | W93227 | W93227 | Hs.103245 | EST |
| 125278 | W93523 | AI218439 | Hs 129998 | enhancer of polycomb 1 |
| 125280 | W93659 | AI123705 | Hs 106932 | ESTs |
| 448205 | W94003 | W93949 | Hs.33245 | ESTs |
| 131844 | W94401 | AI419294 | Hs.324342 | ESTs |
| 125284 | W94688 | NM_002666 | Hs.103253 | perilipin |
| 417111 | W94787 | AW016321 | Hs.82306 | destrin (actin depolymerizing factor) |
| 445424 | Z38294 | AB028945 | Hs.12696 | cortactin SH3 domain-binding protein |
| 125289 | Z38311 | T34530 | Hs.4210 | Homo sapiens cDNA FLJ13069 fis, clone NT |
| 446313 | Z38465 | H06245 | Hs.106801 | ESTs, Weakly similar to PC4259 ferritin |
| 431342 | Z38525 | AW971018 | Hs 21659 | ESTs |
| 433227 | Z38538 | AB040923 | Hs.106808 | kelch (Drosophila)-like 1 |
| 428306 | Z38551 | AB037715 | Hs.183639 | hypothetical protein FLJ10210 |
| 424624 | Z38783 | AB032947 | Hs 151301 | Ca2 + dependent activator protein for secr |
| 125295 | Z39113 | AB022317 | Hs.25887 | sema domain, immunoglobulin domain (Ig), |
| 125298 | Z39255 | AW972542 | Hs.289008 | Homo sapiens cDNA: FLJ21814 fis, clone H |
| 125300 | Z39591 | Z39591 | Hs.101376 | EST |
| 448378 | Z39783 | BE622770 | Hs.264915 | Homo sapiens cDNA FLJ12908 fis, clone NT |
| 444582 | Z39920 | R55344 | Hs 22142 | cytochrome b5 reductase b5R.2 |
| 130882 | Z40166 | AA497044 | Hs.20887 | hypothetical protein FLJ10392 |
| 128888 | Z40388 | AI760853 | Hs.241558 | ariadne (Drosophila) homolog 2 |
| 125310 | Z40646 | R59161 | Hs.124953 | ESTs |
| 125315 | Z41697 | R38110 | Hs.106296 | ESTs |
| 125317 | Z99349 | Z99348 | Hs.112461 | ESTs, Weakly similar to I38022 hypotheti |
| 135096 | Z99394 | AA081258 | | zinc finger protein 36 (KOX 18) |

TABLE 3A

| Pkey | CAT Number | Accession |
|---|---|---|
| 108469 | 116761_1 | AA079487 AA128547 AA128291 AA079587 AA079600 |
| 124106 | 125446_1 | H12245 AA094769 R14576 |
| 108501 | 13684_-12 | AA083256 |
| 108562 | 36375_1 | AA100796 AF020589 AA074629 AA075946 AA100849 AA085347 AA126309 |
| | | AA079311 AA079323 AA085274 |
| 101300 | 4669_1 | BE535511 M62098 AA306787 AW891766 AA348998 AA338869 AA344013 |
| | | AW956561 AW389343 AW403607 L40391 AW408435 AA121738 AI568978 |
| | | H13317 R20373 AW948724 AW948744 AA335023 AA436722 AA448690 C21404 |
| | | AW884390 AA345454 AA303292 AA174174 BE092290 T90614 AA035104 |
| | | R76028 AA126924 AA741086 AW022056 AW118940 AA121666 AI832409 |
| | | AA683475 AI140901 AI623576 AW519064 AW474125 AI953923 AI735349 |
| | | AW150109 AI436154 AW118130 AW270782 AI804073 N27434 AA876543 |
| | | AA937815 AI051166 AA505378 AI041975 AI335355 AI089540 AA662243 |
| | | AI127912 AI925604 AI250880 AI366874 AI564386 AI815196 AI683526 |
| | | AI435885 AI160934 H79030 AI801493 AA448691 AI673767 AI076042 |
| | | AI804327 AA813438 AA680002 AI274492 T16177 AI287337 AI935050 |
| | | AA907805 AA911493 AI589411 AI371358 AW576236 AI078866 AW516168 |
| | | AA346372 AI560185 AA471009 R75857 AA296025 AA523155 AA853168 |
| | | AI696593 AI658482 AI566601 AW072797 AA128047 AA035502 AW243274 |
| | | AA992517 R43760 |
| 132091 | 94851_1 | AW954243 AA829930 AA412478 AA828434 AA814538 AI927418 AI192435 |
| | | W52897 AA443666 AA031913 AI683306 AA918481 AI183314 D83907 |
| | | AI206832 AA876122 D83836 D83838 D82533 AI761290 AI191125 |
| | | AI143749 AW771909 AI241436 AI767267 W56507 AA847787 AA568692 |
| | | T10502 AI247870 AA715017 AA643304 AA890233 AA811387 AA897470 |
| | | AA907729 AI708679 AI078010 AA452830 AW419160 AI783713 N80205 |
| | | W56778 AA676899 AI888718 N69930 AI338935 AI217580 AA639508 |
| | | AA575836 BE046852 AI312651 AI038406 AA628649 AA643838 AI493761 |
| | | AA032024 W38849 AA340178 AA447052 AA452969 W19369 AA296364 |
| | | H44229 W58767 C05751 C05835 AI741989 N98532 AW102617 AA412583 |
| | | AI922246 W38495 AA355375 AA928571 C06275 AA352500 N93132 |
| 117034 | 20113_1 | U72209 NM_005748 AI655607 AI052758 AA385199 AW956794 H88679 |
| | | AL135153 AI765644 AA384399 AW966458 AA568443 AA804610 AI873513 |
| | | H88639 Z25371 R63456 W44919 |
| 100752 | 33207_21 | T81309 BE019033 R94181 BE019198 NM_000612 J03242 AW411299 |
| | | BE300064 BE297544 R94182 AW630108 T53723 D58853 H78073 H80594 |
| | | BE299560 T48899 H70196 M17426 N77077 S77035 H58384 H61664 |
| | | H78540 T84527 C17198 H60255 H71980 R92644 W79050 X00910 M29645 |
| | | R91055 M17863 M17862 T71815 BE299561 BE464561 X06260 R94741 |
| | | T54216 C18594 BE262015 X06161 AW409889 AA378400 BE263228 |
| | | BE313278 R88116 BE313457 H43500 T48617 BE313761 H77309 |
| | | AI207601 X06159 H40413 X03425 T87663 R10627 X03562 M14118 |
| | | W03982 R97520 H81229 T83157 H83168 H48762 AA669898 BE263054 |
| | | H47289 AA022807 R11555 H74260 R76968 R28338 H72534 H72464 |
| | | H62031 N72478 N45355 AW411300 R89113 R69135 H58454 T83281 |
| | | R93476 H69645 H68015 T82229 H71089 T85121 H59939 W65299 N78176 |
| | | H53909 N72373 R21788 H04660 H59639 H61874 BE262219 T53614 |
| | | N73335 N50464 W00943 N77189 R89257 AA570502 R89432 R06366 |
| | | AA553480 AA776271 AA551359 AA551050 H51670 AA601052 BE299081 |
| | | H68198 H52276 BE207832 N91192 H70332 X07868 X07868 H69464 |
| | | H53782 H73710 R80435 AA553384 AW884176 N53475 T71662 AW954036 |
| | | AW954033 AA552931 H93206 AA430218 AA553476 AI918470 T54124 |
| | | BE207982 BE300177 N73994 AW882625 N39549 N53838 AA722389 |
| | | H71878 H58909 H37849 H78435 T47933 R77174 R83814 AA411890 |
| | | H94199 AA663208 BE205778 AA490137 H70492 R98232 H37800 AA679294 |
| | | H40341 H74238 H47290 H73231 T48618 AA025428 AI039521 H92969 |
| | | N59389 H80538 H72933 T90630 AA411891 N55000 H74225 AA340290 |
| | | AW957061 T54316 AA340437 H57125 H58908 H79027 H63450 N74623 |
| | | R93425 H68714 H68758 N68396 H48763 N69256 H57320 H53831 H53589 |
| | | N68833 N52453 H56048 H69870 H78074 R69253 R83375 T53615 H94330 |
| | | H58455 H90864 T47934 H74261 R89258 R97997 R91056 R28339 R86760 |
| | | H78235 R97521 H67692 H40358 AA022688 H52513 H59601 T88690 H65256 |
| | | H63397 W65397 AA553588 R19280 N52645 W73930 R06367 R21743 H72372 |
| | | N73921 AW883539 AW882639 T40616 H47084 R95723 AA634316 AA862781 |
| | | H77310 R91389 H93111 R92767 T54512 R89341 H70333 H57817 H82941 |
| | | H62032 N52638 H58385 T91796 H51086 AA340292 T49918 H81230 R36121 |
| | | N50411 T87664 N62436 N39340 AA665637 AA340446 H93377 H92973 |
| | | BE296290 BE269788 H61665 AA340444 N54605 AA454101 R10628 R94200 |
| | | AI200549 AA342640 BE298855 BE250229 T49916 H82008 N28278 |
| | | AW880662 H71268 N76791 H47685 H65255 W05198 AW889144 N76677 |
| | | H71702 H68036 H71915 R91612 R87807 H68059 AI133328 AI247866 |
| | | AA621443 AW881050 AA700847 AA340413 AW878608 AW881181 AW878249 |
| | | H71916 N54596 BE161581 AW878082 W04212 AW881040 AW885492 |
| | | AW880519 AA334887 AW878715 W06882 AW630222 AW885381 H70869 |
| | | AW381778 H47601 AW889982 H63868 AW884986 AW878713 AW878685 |
| | | R36391 AW878694 AA368070 C03393 AW878695 AW878705 AW878665 |
| | | AW878742 AW878620 AW878823 AW878688 R29048 AW878690 AW878686 |
| | | AW878810 AW878827 AW878733 AW878659 AW878749 AW878681 AW883353 |
| | | AW883277 AW883300 AW883565 AW883298 AW883143 AW883045 AW883482 |
| | | AW883352 AW883417 AW883357 AW883231 AW883474 AW883355 AW882620 |
| | | AW882533 AW883754 AW883139 AW882827 AW883641 AW883567 AW883481 |
| | | AW882983 AW882982 AW882465 AW883419 AW882466 AW883639 AW883230 |
| | | AW882981 AW882534 AW882874 AW882619 AW883480 AW882826 AW882831 |
| | | AW882835 AW882830 AW883563 AW882456 AW627642 |
| 116417 | 5418_11 | AW499664 AW500888 AL042095 AW576556 AW265424 AI521500 AA761333 |
| | | AA761319 AW291137 AA649040 AA769094 AA489664 AA635311 AW070509 |
| | | AA425658 AI381489 AA609309 AA134476 W74704 AI923640 AW084888 |
| | | H45700 AI985564 AW629495 AW614573 AI859571 AI693486 AA913892 |
| | | AI806164 AA909524 AW263513 AI356361 Z40708 AI332765 AI392620 |
| | | AA181060 AW118719 AW968804 AW263502 AW505314 AA036967 W74741 |
| | | R51139 H19364 H45751 Z44962 AW370823 H25650 T54007 AA453000 |
| | | AL045739 |
| 123712 | 374423_1 | AA609684 AA758732 |
| 117156 | 145392_1 | W73853 M928112 W77887 AW889237 AA148524 AI749182 AI754442 |
| | | AI338392 AI253102 AI079403 AI370541 AI697341 H97538 AW188021 |
| | | AI927669 W72716 AI051402 AI188071 AI335900 N21488 AW770478 |
| | | W92522 AI691028 AI913512 AI144448 W73819 AA604358 N28900 |
| | | W95221 AI868132 H98465 AA148793 |
| 125008 | 1802095_1 | T91251 T64891 T85665 |
| 125020 | 116017_1 | T69981 T69924 AA078476 |
| 125066 | 1814993_1 | T86284 T81933 |
| 116661 | 1532859_1 | R61504 F04247 |
| 125104 | 413347_1 | T95590 AA703278 H62764 |
| 124575 | 1666649_1 | N68168 N69188 N90450 |
| 125263 | 1547_2 | AA098878 W88942 |
| 131859 | 3672_1 | AW960564 AA092457 T55890 D56120 T92525 AI815987 BE182608 |
| | | BE182595 AW080238 M90657 AA347236 AW961686 AW176446 AA304671 |
| | | AW583735 T61714 AA316968 AI446615 AA343532 AA083489 AA488005 |
| | | W52095 W39480 N57402 D82638 W25540 W52847 D82729 D58990 |
| | | BE619182 AA315188 AA308636 AA112474 W76162 AA088544 H52265 |
| | | AA301631 H80982 AA113786 BE620997 AW651691 AA343799 BE613669 |
| | | BE547180 BE546656 F11933 AA376800 AW239185 AA376086 BE544387 |
| | | BE619041 AA452515 AA001806 AA190873 AA180483 AA159546 F00242 |
| | | AI940609 AI940602 AI189753 T97663 T66110 AW062896 AW062910 |
| | | AW062902 AI051622 AI828930 AA102452 AI685095 AI819390 AA557597 |
| | | AA383220 AI804422 AI633575 AW338147 AW603423 AW606800 AW750567 |
| | | AW510672 AI250777 AA083510 AW629109 AW513200 AA921353 AI677934 |
| | | AI148698 AI955858 AA173825 AA453027 AI027865 AW375542 AA454099 |
| | | AA733014 AI591384 R79300 R80023 AA843108 AA626058 AA844898 |
| | | AW375550 AA889018 AI474275 AW205937 AI052270 AW388117 AW388111 |
| | | AA699452 AI242230 N47476 H38178 AA366621 AA113196 AA130023 |
| | | H39740 T61629 AI885973 AW083671 AA179730 AA305757 AI285455 |
| | | N83956 AA216013 AA336155 AW999959 T97525 AA345349 T91762 |
| | | AA771981 AI285092 AI591386 BE392486 BE385852 AA682601 AI682884 |
| | | AA345840 T85477 AA292949 AA932079 AA098791 D82607 T48574 AW752038 |
| | | C06300 |
| 125565 | 1704098_1 | R20840 R20839 |
| 132983 | 11922_1 | M30269 NM_002508 X82245 AI078760 AW957003 D78945 M27445 AA650439 |
| | | AL048816 AV660256 AV660347 AA333052 BE295257 T60999 AA383049 |
| | | AW369677 Z26985 AW175704 AA343326 AW747957 AI818389 W17308 |
| | | W17302 H15591 AA371284 AA370412 W94966 BE384365 T28498 R80714 |
| | | R16959 H21723 AW835154 D56097 D56381 W21232 AA190565 AW379755 |
| | | AW067895 |
| 118584 | 532052_1 | AW136928 AI685655 BE218584 BE465078 N68963 AA975338 BE147199 |
| | | N76377 |
| 133607 | 1227_6 | BE273749 BE397561 BE387189 AL037858 AL037878 AI963094 BE259216 |
| | | AA011363 AL036189 BE562325 AA251169 BE617431 N98537 AA158093 |
| | | AL047800 M34539 NM_000801 AA312140 D16971 AA158904 AA307114 |
| | | AA312803 T09203 AW629686 AL048504 BE388578 AA220957 AA158364 |
| | | BE267385 AA294971 C18055 BE241757 AA115056 AI936769 BE378435 |
| | | BE206971 AW674924 BE622060 AA604674 AA115273 AW402159 AA338608 |
| | | BE568819 M80199 X55741 AA375111 AA376016 BE612671 AA805742 |
| | | AW405588 N25850 N44580 H06031 AW403549 BE536552 AA056726 |
| | | BE543239 AA082517 AI201645 AI201642 AI192622 N40104 AA370921 |
| | | BE547569 AI969602 AA302038 AI197890 AW268354 AI014938 W45448 |
| | | AI541395 AA037272 BE538826 AL039613 BE536130 AA299355 AW805147 |
| | | AW974624 H53220 AI471471 AA399303 AA007386 W35106 BE613277 |
| | | R12739 R12738 AA304342 AA687802 BE409581 AI498844 AV662092 |
| | | AW904105 AA011375 BE315214 H99302 BE537893 N32299 AW855829 |
| | | AI291320 BE078322 AI301395 AA303362 N32719 AA358328 AA357877 |
| | | AI952540 H56279 H02758 H02048 AW805233 R82224 AA410772 AA291352 |
| | | BE171109 N69935 BE169248 AA361173 H44978 BE617887 D52560 |
| | | AA084043 W03595 R67219 N36477 N42924 R67104 H44901 H79695 |
| | | W21105 AA393988 W30899 AA316096 BE622896 W46872 AA442678 |
| | | BE544893 BE540112 BE621873 AA338067 N55052 BE398154 BE621210 |
| | | AA740760 C03739 C03206 BE396692 AA482370 AA031614 AA301575 |
| | | AA304710 AA132153 AA029796 AA994960 H19567 AA442969 H49781 |
| | | H46871 AA035395 AA056185 AA149378 AA643080 AL135479 AA292329 |
| | | AA654337 AA041228 AA454888 AA025039 W58331 AA625981 T94941 |
| | | AA302448 H19900 AA218956 AA513790 AA563962 AA398076 W44441 |
| | | AA293276 W47373 AA625879 W30688 AA043029 T64284 R79151 AA304340 |
| | | AA485186 AA604939 R82470 AA421425 AW771456 AI339329 AA304424 |
| | | AA605236 AA936934 AA587673 AI209162 AI697301 AI479995 AI679814 |
| | | AI361950 AW189125 AI955888 AI986019 BE301019 AI084792 AI310211 |
| | | AW189307 AI022070 AW977204 AI146825 AW190163 AW303281 AI828345 |
| | | BE046043 AW029257 AA482268 AI246507 AI420729 AW084932 AW439514 |
| | | AI890487 AW439692 AI523896 AI186612 AI659953 AI889773 AA687527 |
| | | AW072694 AW262153 AW467371 AI613269 AI679238 D54404 AA158103 |
| | | AW105527 AW149739 AW150361 AW268387 AW117708 AI951682 AI687440 |
| | | AW674285 AA678365 AI587082 AA732095 AA019899 W45661 AA627300 |
| | | BE613304 AA765891 AA612935 AI814658 AW316916 R66594 AA514640 |
| | | AA025040 AA031472 AW732076 AA029797 AI244560 AI128734 AW381720 |
| | | AI092360 AI263283 AW613175 AI890675 AI720156 AW631348 AI635106 |
| | | AI278045 AA303979 AA703505 W45449 AW078661 AI292052 AW381707 |
| | | AI147854 AW381743 AA158905 AA303258 AA888144 AW195967 AA428706 |
| | | AA989559 AA617731 H19882 BE543418 AA830386 AA421302 W58652 |
| | | T94995 AI869743 AI679145 AW085971 N98425 AA765136 AI347027 |
| | | AI356955 AA928038 AI679717 AA458459 AA679281 AI367973 AI270041 |
| | | AA765135 AA732793 AI798447 AA668646 AA251008 AI984538 AI401737 |
| | | AA056186 BE043308 AW662375 AI302110 N50724 W96332 BE537047 |
| | | N26983 AI567172 AA765296 AW673237 N29784 AA534275 AA084044 |
| | | AW067973 AW300766 T63398 W46823 R39790 AI364185 AW298582 |
| | | AA454814 AW069878 N67751 H05982 N23140 AI362647 AI302086 |
| | | AI767772 N25755 H53114 AA706133 T93511 AA429291 AA935294 |
| | | AA987647 W02803 R66595 AI680795 W23673 AW440794 AA722872 |
| | | H49538 AW131042 AA531603 AA908665 AA040791 AA235312 W52205 |
| | | N93444 R82180 H02759 H79696 AW088894 H56079 AA961143 AW067776 |
| | | AW973745 AA016311 AW071227 AA017511 AI753994 W47374 T64155 |
| | | AA296092 AI698626 AA558158 AA296088 AW794259 H01963 AA149267 |
| | | AA485076 AA975856 H44938 AA035396 AI955555 H46289 AA486161 |
| | | AI631222 AA359047 AW794253 AI806962 AW243930 AA526145 AW878734 |
| | | AA018464 AA132031 R67220 R79152 AA296093 H54300 AI005160 |
| | | BE242548 AW992803 AW878644 AW878666 T27742 R82471 AW517604 |
| | | AW472738 AI282904 R39791 AA486098 AW467891 AW960520 AA551736 |
| | | AA056621 AW945197 R66373 AA554236 BE242202 AI904376 AI832590 |
| | | H19484 R00890 AI627677 AA302287 AI869451 AI734855 AI708073 |
| | | AI832902 AA585184 AW204299 AA055565 D12417 D11975 T63543 |
| | | AW664099 R54423 BE612712 T96340 T63985 AA598917 T40735 T64053 |
| | | AA149284 AW272548 AA363445 AA042893 AW300697 BE261973 T53501 |
| | | T53500 AW878729 AW878657 AW794391 AA069193 R01553 H44875 |
| | | AA385406 AA533968 M93060 AL135600 W96331 AA017651 AA018849 |
| | | AA017692 H85337 BE278690 AA731598 AA018512 AI076813 AI022644 |
| | | R02585 X52220 AW296894 AA825671 AI699321 AI393601 AW592611 |
| | | AI146747 AA608921 AA158365 AW590007 AA354519 D20081 R02704 |
| | | AW798339 M92422 AA094903 AA007676 |
| 133681 | 13893_1 | AI352558 Z82248 X78138 NM_003405 AU077248 AA223125 S80794 D78577 |
| | | AI124697 AW403970 BE614089 BE296713 BE621334 L20422 X80536 |
| | | D54224 D54950 X57345 N29226 AA127798 AA340253 F08031 AA192540 |
| | | H67636 AA321827 AW950283 AA084159 BE538808 AW401377 AA256774 |
| | | C03366 W46595 W47608 AA305009 H69431 H69456 AL120082 H11706 |
| | | AA303717 AA361357 H22042 H78020 AW999584 AA134368 AA322911 |
| | | AA322961 H60980 N85248 N31547 H79624 T11718 W85826 AW894663 |
| | | AW894624 BE167441 BE170015 AA304626 AW602163 AW998929 AA156681 |
| | | AA151067 BE002724 AA608688 H82692 BE155392 AW383636 BE155394 |
| | | AA487004 AW383504 AI342365 R82553 W16498 BE155344 AI143938 |
| | | R69901 AA322873 AW340648 R25364 AA367935 AI559406 AA033522 |
| | | AA374252 AW835019 AI922133 AI697089 N99662 AW189078 AI199076 |
| | | AW151598 W59944 AA662875 W94022 AA299055 AI039008 AI829449 |
| | | AA583503 AI635674 AW131665 AI473820 AW273118 AW900930 AA908944 |
| | | AI688035 AW170272 AI082545 AW468176 AI608761 AI082748 AI911682 |
| | | AI248943 AI831016 AA192465 AI218477 AA938406 AA385288 AI809817 |
| | | AA905196 AI191245 AI470204 AI188296 AI421367 AI125315 AI087141 |
| | | AA629032 AA740589 AI554181 AA150830 AI248541 AI077943 AA775958 |
| | | AA864930 AI261476 AI123121 AI310394 AA862331 AA872478 BE537084 |
| | | AI205606 AA720684 AI872093 AW150042 AL120538 AA219627 AA988608 |
| | | C21397 AI359337 H25337 AI089749 AA605146 AI359620 AA150478 |
| | | AI359738 AW383642 AW995424 AI766457 R56892 AI089839 W61343 |
| | | N69107 W46459 AA565955 N20527 AI279782 W46596 AA776573 H23204 |
| | | AI866231 AI083995 N21530 AA126874 D82630 W65437 AI086917 |
| | | AW382095 AI086877 H69844 AW340217 W85827 L08439 AA262704 |
| | | AA505380 W47413 W94135 AA223241 AW089153 AA084101 BE538000 |
| | | AA096126 T28031 AA491574 R84813 AA774536 AW383522 AA155615 |
| | | AW383529 AA491520 AW028427 AA171496 AI469689 AW664539 AI811102 |
| | | AI811116 BE464590 BE350791 H78021 T15405 H21979 AA219489 H13301 |
| | | AA505883 AI864305 AI423963 AW084401 F04963 R69858 H67097 |
| | | AI917740 AI655561 H69864 AA033631 AW383484 AI886261 H25293 |
| | | AA513281 AW271187 H11617 N79982 AI174338 AI904207 AI904208 |
| | | BE614558 W94127 W65436 AI272249 AA700018 AI579932 AI085941 |
| | | AW152629 |
| 134403 | 17037_1 | AA334551 BE008229 AA307537 AW961156 AW995894 AW995826 |
| | | NM_006751 M61199 AA045603 AL036372 AV645606 AI688095 AW351901 |
| | | AA101337 AA101345 N73342 BE018030 BE569044 AW841975 AA373388 |
| | | BE090412 H95440 N53845 R67867 AA093441 AA363427 H93708 AW023134 |
| | | AW994986 AW994989 BE090429 R23614 AI567932 H03726 H01101 |
| | | H01867 AA548743 AI671806 AW872949 AW872941 AA742447 AI199788 |
| | | AA045604 AI637465 AI741796 AW242217 AW131463 AI765302 AI683923 |
| | | AA889762 AI804889 AI986437 C06049 BE502340 AI695651 AI491970 |
| | | AA496804 AA281008 AA665699 AI473814 BE301445 AA707837 AA551925 |
| | | AI017348 AI208185 AA775203 AA156296 AA557463 H95441 AA768547 |
| | | AW769358 AA991197 AA181954 AI091389 AI147289 AW771837 AI638582 |
| | | AA844411 AI374750 T29320 AW951272 AW085923 H02834 AA843259 |
| | | AA814696 AW183290 AA158453 N68125 N69039 AA100423 AA101346 |
| | | AI918720 H01102 R67868 H01868 N66438 R46580 AI858433 AA599560 |
| | | AA187577 AA157481 AA361520 AL047827 AA158452 R21688 AW964874 |
| | | AA325161 R40871 AW752395 AW375924 R13355 AA281174 AA428908 |
| 135096 | 33756_2 | AA081258 AA160311 W17034 H83596 Z99393 AI831206 AW771108 |
| | | AW769214 N89775 AW161495 AW161522 AW160880 Z99394 AI814820 |
| 103767 | 34817_1 | BE244667 BE241813 BE242271 AA381943 NM_016040 AF151858 AW967497 |
| | | AW966873 AI824386 AW470133 AW015765 BE018650 AW503659 AI129838 |
| | | AI632346 AA013099 AW770511 BE219482 AI824135 AI867379 AA019348 |
| | | AA285143 AW087624 AI990100 AA251084 AI633962 AA287714 AA400773 |
| | | AI292112 AW469095 AA743312 AW117423 AA694551 AA885657 AA112675 |
| | | BE327333 AA082161 H03613 AA094735 AW500235 N28878 AA287713 |
| | | AW300233 AA826249 N46921 BE348728 AW505056 AW966879 AI521202 |
| | | AA393405 AI264668 AA910851 AA251721 AI470834 H03503 AA089688 |
| | | R58562 BE004728 AA668793 H27167 R54717 |
| 103855 | 84277_1 | W02363 N80298 AA304486 AW954799 AW805136 AW970817 AW373398 |
| | | AW875459 AA136805 AA683501 N73299 AW341082 AI632954 AA493369 |
| | | AI478433 AI037911 AW272169 AW043832 AA010683 AW629090 AW183622 |
| | | N64510 AW079953 AI554533 AA563670 AA010682 AW237610 AW419057 |
| | | AI470926 AI627833 AA195080 AA195179 AI471443 AW590266 AI168477 |
| | | AW771214 AI767341 AW340086 AW748455 AI280079 AI244821 AI381283 |
| | | AW300130 AW183374 AW195397 AA136706 AI824598 AW573004 Z98448 |
| | | AA905255 AI497883 |
| 126872 | 142696_1 | AW450979 AA136653 AA136656 AW419381 AA984358 AA492073 BE168945 |
| | | AA809054 AW238038 BE011212 BE011359 BE011367 BE011368 BE011362 |
| | | BE011215 BE011365 BE011363 |
| 113026 | 84431_1 | AA376654 W76367 AA318232 AI694545 AI742403 AI887383 AW204731 |
| | | AW874431 BE220997 AA114979 AA303838 AI002267 AW952031 W74801 |
| | | AA011287 AA115112 AI306385 R37677 AW571707 R59986 W94102 |
| | | AW197042 H10206 AW139819 AI686172 AI674165 R51633 AI367086 |
| | | T23948 H10833 H23002 H11743 R37085 Z39208 H22794 H11820 |
| | | R13817 Z43122 H10257 R88398 R18795 AA010848 R67191 H10875 |
| | | R67170 |
| 120284 | 158963_1 | AA179656 AA182626 AA182603 |
| 112540 | 1605263_1 | R69751 R70467 H69771 H80879 H80878 |
| 111904 | 1719336_1 | Z41572 R39330 |
| 121094 | 275729_1 | AA402505 AA398900 |
| 128510 | 19829_1 | X94703 NM_004249 R52316 T87420 N46403 Z36855 BE076834 |
| 114106 | 1182096_1 | AW602528 BE073859 Z38412 |
| 121335 | 279548_1 | AA404418 AI217248 |
| 120761 | 224903_1 | AA321890 R18000 |
| 122050 | 273507_2 | AI453076 AI376075 AI014836 AA628633 AA961066 AI150282 AI028574 |
| | | AI217182 AA732910 AA431478 AL041229 |
| 130018 | 18986_1 | AA353093 AW957317 AW872498 AI560785 AI289110 AW135512 X97261 |
| | | T68873 |
| 100104 | 19974_-3 | AF008937 |
| 121822 | 244391_1 | AI743860 N49543 AW027759 BE349467 AI656284 BE463975 R35022 |
| | | AA370031 AW955302 AL042109 N53092 AI611424 AL079362 AI969290 |
| | | AI928016 BE394912 BE504220 BE467505 AI611611 AI611407 AI611452 |
| | | W56437 AI284566 AI583349 AW183058 AI308085 AI074952 AA437315 |
| | | AA628161 AW301728 AI150224 AA400137 AA437279 AI223355 AA639462 |
| | | AI261373 AI432414 AI984994 AI539335 AA401550 AA358757 AI609976 |
| | | AA442357 AA359393 AA437046 AA370301 AA429328 AW272055 AI580502 |
| | | AI832944 AI038530 AA425107 AI014986 AI148349 AW237721 AW779756 |
| | | AW137877 AI125293 AA400404 R28554 |
| 108280 | 110682_1 | AA065069 AA085108 |
| 108309 | 111495_1 | AA069818 AA069971 AA069923 AA069908 |
| 107832 | genbank_AA021473 | AA021473 |
| 123523 | genbank_AA608588 | AA608588 |
| 123533 | genbank_AA608751 | AA608751 |
| 132225 | genbank_AA128980 | AA128980 |
| 125017 | genbank_T68875 | T68875 |
| 125063 | genbank_T85352 | T85352 |
| 125064 | genbank_T85373 | T85373 |
| 125091 | genbank_T91518 | T91518 |
| 100964 | entrez_J00212 | J00212 |
| 102269 | entrez_U30245 | U30245 |
| 125150 | NOT_FOUND_{—} | W38240 |
| | entrez_W38240 | |
| 123964 | genbank_C13961 | C13961 |
| 118111 | genbank_N55493 | N55493 |
| 118129 | genbank_N57493 | N57493 |
| 102491 | entrez_U51010 | U51010 |
| 118329 | genbank_N63520 | N63520 |
| 118475 | genbank_N66845 | N66845 |
| 118581 | genbank_N68905 | N68905 |
| 111514 | genbank_R07998 | R07998 |
| 104534 | R22303_at | R22303 |
| 120340 | genbank_AA206828 | AA206828 |
| 120376 | genbank_AA227469 | AA227469 |
| 104787 | genbank_AA027317 | AA027317 |
| 120409 | genbank_AA235050 | AA235050 |
| 120745 | genbank_AA302809 | AA302809 |
| 120809 | genbank_AA346495 | AA346495 |
| 120839 | genbank_AA348913 | AA348913 |
| 113702 | genbank_T97307 | T97307 |
| 106596 | 304084_1 | AI583948 AA578212 AW303715 AA653450 AA456981 AI400385 W88533 |
| | | AI224133 AW272145 AA088686 R94698 |
| 113947 | genbank_W84768 | W84768 |
| 122562 | genbank_AA452156 | AA452156 |
| 122635 | genbank_AA454085 | AA454085 |
| 108277 | genbank_AA064859 | AA064859 |
| 108403 | genbank_AA075374 | AA075374 |
| 122860 | genbank_AA464414 | AA464414 |
| 108427 | genbank_AA076382 | AA076382 |
| 108439 | genbank_AA078986 | AA078986 |
| 131353 | 231290_1 | AW411259 H23555 AW015049 AI684275 AW015886 AW068953 AW014085 |
| | | AI027260 R52686 AA918278 AI129462 AA969360 N34869 AI948416 |
| | | AA534205 AA702483 AA705292 |
| 108533 | genbank_AA084415 | AA084415 |
| 124254 | genbank_H69899 | H69899 |
| 101447 | entrez_M21305 | M21305 |
| 101458 | entrez_M22092 | M22092 |
| 101667 | 13349_1 | NM_005381 M60858 AW373732 AW373724 AW373689 AW373629 AW373609 |
| | | AW373776 AA187806 AW386946 AW374207 T05235 AA216203 AW385556 |
| | | AA306940 AA306526 AA315461 AL036757 AW373711 AW403124 AW403640 |
| | | AW377084 T27360 H62638 F06957 AW377051 AA554779 AA378568 |
| | | AA096007 AW352407 AW302637 F07929 H17433 AW382712 H05665 |
| | | F07292 N39875 AA089729 H62556 N42842 R12952 AW373735 AW364155 |
| | | AA056183 W39185 AW382708 N32488 AF114096 AW375993 AI133569 |
| | | W52561 AA603040 AA133710 AI928796 AW176370 AA827519 AW338437 |
| | | AA521142 T29341 AI800461 AW317002 AA703914 AA860830 AI859203 |
| | | AI445772 AA714334 AI817066 AI832027 AW510442 AI635802 AW088306 |
| | | AW068672 AW408555 AW467542 AA552657 AA152367 W32081 AA582124 |
| | | AA074040 AA931657 AI051154 AW410203 AI921644 H17434 AI832330 |
| | | AW404836 AI925038 AA088423 AA954166 AA580453 AW021292 AI267215 |
| | | AW080082 AW383778 AI933053 AI919097 W31557 N90245 AA931591 |
| | | AA563995 F36352 AA056184 AA476294 AA641327 AA533550 AI749630 |
| | | W58323 AA569119 AA508573 AI809050 AI378996 AA411362 AW407505 |
| | | AA938104 AA074041 AA632876 AW193748 AA507873 AI270128 AI472365 |
| | | AA411363 AI523216 AI719965 AI816302 AA182681 AI707990 AA133588 |
| | | AI758537 W60253 AI460308 AA135423 AI083904 F04188 N89693 |
| | | AW408776 AI678595 AI270568 AA722059 W58234 F33650 AA090547 |
| | | AA285108 AA425981 N85079 D20218 AI273980 AA159028 F03226 |
| | | AW247914 N26918 AW272741 N90109 H05666 N23327 AW247953 R44748 |
| | | AA962015 F03558 AI752394 AW409913 AW248396 AI816463 AI752393 |
| | | AA325370 AA263089 AI570130 AI971951 AI160658 AI357360 AW168686 |
| | | AL121075 AW050536 N21672 W67748 AA514242 AI127386 H14607 |
| | | AI185752 W79364 AA088520 AA152476 AW351940 AW373683 AI940524 |
| | | AW374953 T56500 N24329 AI940720 AW374933 AW374947 AW391913 |
| | | AL138337 AW376241 AW062943 F26666 AW410202 AW062958 F34529 |
| | | AW381807 AW393315 W17147 AW176359 AA664576 AW380424 AA306040 |
| | | AI745674 AW300951 AI188579 AI438973 AI305271 AA433818 M612807 |
| | | AI831809 AI940409 AA158663 AI572988 |
| 124576 | genbank_N68201 | N68201 |
| 108931 | genbank_AA147186 | AA147186 |
| 108941 | genbank_AA148650 | AA148650 |
| 124720 | 144582_1 | R05283 R11056 |
| 124793 | genbank_R44519 | R44519 |
| 124799 | genbank_R45088 | R45088 |
| 103138 | entrez_X65965 | X65965 |
| 117683 | genbank_N40180 | N40180 |
| 124991 | genbank_T50116 | T50116 |
| 103432 | entrez_X97748 | X97748 |
| 119174 | genbank_R71234 | R71234 |
| 119239 | 95573_2 | T11483 T11472 |
| 133678 | 11235_1 | AW247252 AA346143 NM_000270 AA381085 N91995 X00737 AA381079 |
| | | AA296473 AA296110 AA315735 AA311617 AA326750 AA376804 AW403290 |
| | | T95231 M13953 T47963 H82039 AA279899 AA627997 N76320 N99527 |
| | | H37842 W20095 AA457308 AW469547 AA724143 H83220 AA319496 W86334 |
| | | W30892 R89169 R99427 N41854 H47286 AA348094 AA045089 R63016 |
| | | AI922219 AI024906 AI096488 AI885005 AA194872 N90489 AI452544 |
| | | H72411 AA282427 AA430735 R68963 R22453 H70385 AW129369 AW467320 |
| | | AW519082 AA345018 AA582183 AI961789 R65918 N30611 AI979189 |
| | | AI280889 AW273191 R66531 AI285845 AI675927 AI421990 AW190879 |
| | | H37794 AA699667 H68427 AA954388 AI188757 AI140048 AA430382 |
| | | AI204151 AW247864 AA559099 AI431420 AA548276 AI149466 AA772669 |
| | | AA694388 AA724168 AA301651 AA281952 AA779925 AA234760 W86290 |
| | | AA913603 AW511745 AI500697 AA814922 AA835040 T47964 H53998 |
| | | AA975804 R98710 AI077604 N70252 R98084 AW250171 H69268 AI597614 |
| | | AA970746 AA972548 AI377116 R62962 H16737 R89070 AA731329 R66532 |
| | | N54354 AI818832 H81944 N71567 T95122 W86463 AA437095 AI431999 |
| | | AI915724 N63851 AI674743 AA457307 AA211475 N64444 AI799146 |
| | | H72853 R99335 H60413 AA770367 AA156105 AI269937 H64029 H89728 |
| | | R65819 AW470496 AI873318 AI735713 H82987 C02447 AI478666 T27651 |
| | | AI699770 AW025156 H69719 AI984717 N69225 AI459856 AA953577 |
| | | AI424691 H13843 R22404 AI873796 AI336002 N70898 AI420854 |
| | | AA541792 AA346142 AI000814 AI828348 AA045090 T51257 N90434 |
| | | H13890 N73184 AI708083 AA781606 AA329050 AA339985 R68964 H64795 |
| | | W04186 H16845 |
| 119416 | genbank_T97186 | T97186 |
| 119558 | NOT_FOUND_{—} | W38194 |
| | entrez_W38194 | |
| 119559 | NOT_FOUND_{—} | W38197 |
| | entrez_W38197 | |
| 119654 | genbank_W57759 | W57759 |
| 121350 | genbank_AA405237 | AA405237 |
| 121558 | genbank_AA412497 | AA412497 |
| 105985 | genbank_AA406610 | AA406610 |
| 114648 | genbank_AA101056 | AA101056 |
| 121895 | genbank_AA427396 | AA427396 |
| 100327 | entrez_D55640 | D55640 |
| 123315 | 714071_1 | AA496369 AA496646 |
| 123473 | genbank_AA599143 | AA599143 |
| Table 3A shows the accession numbers for those pkeys lacking unigeneID's for Table 3. The pkeys in Table 7 lacking unigeneID's are represented within Tables 1-6A. For each probeset we have listed the gene cluster number from which the oligonucleotides were designed. Gene clusters were compiled using sequences derived from Genbank ESTs and mRNAs. These sequences were clustered based on sequence similarity using Clustering and Alignment Tools (DoubleTwist, Oakland California). | | |
| # The Genbank accesssion numbers for sequences comprising each cluster are listed in the “Accession” column. | | |

Pkey: Unique Eos probeset identifier number
CAT number: Gene cluster number
Accession: Genbank accession numbers

TABLE 4

| Pkey | Accession | ExAccn | UniGene | UnigeneTitle |
|---|---|---|---|---|
| 100405 | D86425 | AW291587 | Hs.82733 | nidogen 2 |
| 100420 | D86983 | D86983 | Hs.118893 | Melanoma associated gene |
| 100481 | HG1098-HT1098 | X70377 | Hs.121489 | cystatin D |
| 100484 | HG1103-HT1103 | NM_005402 | Hs.288757 | v-ral simian leukemia viral oncogene hom |
| 100718 | HG3342-HT3519 | BE295928 | Hs 75424 | inhibitor of DNA binding 1, dominant neg |
| 100991 | J03764 | J03836 | Hs.82085 | serine (or cysteine) proteinase inhibito |
| 101097 | L06797 | BE245301 | Hs 89414 | chemokine (C—X—C motif), receptor 4 (fus |
| 101168 | L15388 | NM_005308 | Hs.211569 | G protein-coupled receptor kinase 5 |
| 101194 | L20971 | L20971 | Hs.188 | phosphodiesterase 4B, cAMP-specific (dun |
| 101261 | L35545 | D30857 | Hs.82353 | protein C receptor, endothelial (EPCR) |
| 101345 | L76380 | NM_005795 | Hs.152175 | calcitonin receptor-like |
| 101447 | M21305 | M21305 | | gb:Human alpha satellite and satellite 3 |
| 101485 | M24736 | AA296520 | Hs.89546 | selectin E (endothelial adhesion molecul |
| 101543 | M31166 | M31166 | Hs 2050 | pentaxin-related gene, rapidly induced b |
| 101550 | M31551 | Y00630 | Hs 75716 | serine (or cysteine) proteinase inhibito |
| 101560 | M32334 | AW958272 | Hs.347326 | intercellular adhesion molecule 2 |
| 101674 | M61916 | NM_002291 | Hs.82124 | laminin, beta 1 |
| 101714 | M68874 | M68874 | Hs.211587 | phospholipase A2, group IVA (cytosolic, |
| 101741 | M74719 | NM_003199 | Hs.326198 | transcription factor 4 |
| 101838 | M92934 | BE243845 | Hs.75511 | connective tissue growth factor |
| 101857 | M94856 | BE550723 | Hs.153179 | fatty acid binding protein 5 (psoriasis- |
| 102012 | U03057 | BE259035 | Hs.118400 | singed (Drosophila)-like (sea urchin fas |
| 102024 | U03877 | AA301867 | Hs.76224 | EGF-containing fibulin-like extracellula |
| 102164 | U18300 | NM_000107 | Hs 77602 | damage-specific DNA binding protein 2 (4 |
| 102241 | U27109 | NM_007351 | Hs.268107 | multimerin |
| 102283 | U31384 | AW161552 | Hs 83381 | guanine nucleotide binding protein 11 |
| 102303 | U33053 | U33053 | Hs.2499 | protein kinase C-like 1 |
| 102564 | U59423 | U59423 | Hs 79067 | MAD (mothers against decapentaplegic, Dr |
| 102663 | U70322 | NM_002270 | Hs.168075 | karyopherin (importin) beta 2 |
| 102759 | U81607 | NM_005100 | Hs.788 | A kinase (PRKA) anchor protein (gravin) |
| 102778 | U83463 | AF000652 | Hs 8180 | syndecan binding protein (syntenin) |
| 102804 | U89942 | NM_002318 | Hs.83354 | lysyl oxidase-like 2 |
| 102887 | X04729 | J03836 | Hs.82085 | serine (or cysteine) proteinase inhibito |
| 102898 | X06256 | NM_002205 | Hs.149609 | integrin, alpha 5 (fibronectin receptor, |
| 102915 | X07820 | X07820 | Hs.2258 | matrix metalloproteinase 10 (stromelysin |
| 103036 | X54925 | M13509 | Hs.83169 | matrix metalloproteinase 1 (interstitial |
| 103037 | X54936 | BE018302 | Hs.2894 | placental growth factor, vascular endoth |
| 103095 | X60957 | NM_005424 | Hs.78824 | tyrosine kinase with immunoglobulin and |
| 103158 | X67235 | BE242587 | Hs.118651 | hematopoietically expressed homeobox |
| 103166 | X67951 | AA159248 | Hs.180909 | peroxiredoxin 1 |
| 103185 | X69910 | NM_006825 | Hs.74368 | transmembrane protein (63 kD), endoplasmi |
| 103280 | X79981 | U84722 | Hs.76206 | cadherin 5, type 2, VE-cadherin (vascula |
| 103554 | Z18951 | AI878826 | Hs.74034 | caveolin 1, caveolae protein, 22 kD |
| 103850 | AA187101 | AA187101 | Hs.213194 | hypothetical protein MGC10895 |
| 104465 | N24990 | Z44203 | Hs.26418 | ESTs |
| 104592 | R81003 | AW630488 | Hs.25338 | protease, serine, 23 |
| 104764 | AA025351 | AI039243 | Hs 278585 | ESTs |
| 104786 | AA027168 | AA027167 | Hs.10031 | KIAA0955 protein |
| 104850 | AA040465 | AL133035 | Hs.8728 | hypothetical protein DKFZp434G171 |
| 104865 | AA045136 | T79340 | Hs 22575 | B-cell CLL/lymphoma 6, member B (zinc fi |
| 104894 | AA054087 | AF065214 | Hs 18858 | phospholipase A2, group IVC (cytosolic, |
| 104952 | AA071089 | AW076098 | Hs.345588 | desmoplakin (DPI, DPII) |
| 104974 | AA085918 | Y12059 | Hs.278675 | bromodomain-containing 4 |
| 105178 | AA187490 | AA313825 | Hs.21941 | AD036 protein |
| 105263 | AA227926 | AW388633 | Hs.6682 | solute carrier family 7, (cationic amino |
| 105330 | AA234743 | AW338625 | Hs.22120 | ESTs |
| 105376 | AA236559 | AW994032 | Hs.8768 | hypothetical protein FLJ10849 |
| 105729 | AA292694 | H46612 | Hs.293815 | Homo sapiens HSPC285 mRNA, partial cds |
| 105826 | AA398243 | AA478756 | Hs.194477 | E3 ubiquitin ligase SMURF2 |
| 105977 | AA406363 | AK001972 | Hs.30822 | hypothetical protein FLJ11110 |
| 106008 | AA411465 | AB033888 | Hs.8619 | SRY (sex determining region Y)-box 18 |
| 106031 | AA412284 | X64116 | Hs.171844 | Homo sapiens cDNA: FLJ22296 fis, clone H |
| 106124 | AA423987 | H93366 | Hs.7567 | Homo sapiens cDNA: FLJ21962 fis, clone H |
| 106155 | AA425309 | AA425414 | Hs.33287 | nuclear factor I/B |
| 106302 | AA435896 | AA398859 | Hs.18397 | hypothetical protein FLJ23221 |
| 106423 | AA448238 | AB020722 | Hs.16714 | Rho guanine exchange factor (GEF) 15 |
| 106793 | AA478778 | H94997 | Hs.16450 | ESTs |
| 107174 | AA621714 | BE122762 | Hs.25338 | ESTs |
| 107216 | D51069 | D51069 | Hs.211579 | melanoma cell adhesion molecule |
| 107295 | T34527 | AA186629 | Hs 80120 | UDP-N-acetyl-alpha-D-galactosamine polyp |
| 107385 | U97519 | NM_005397 | Hs.16426 | podocalyxin-like |
| 108756 | AA127221 | AA127221 | Hs.117037 | ESTs |
| 108846 | AA132983 | AL117452 | Hs.44155 | DKFZP586G1517 protein |
| 108888 | AA135606 | AA135606 | Hs.189384 | gb:zl10a05.s1 Soares_pregnant_uterus_NbH |
| 109001 | AA156125 | AI056548 | Hs.72116 | hypothetical protein FLJ20992 similar to |
| 109166 | AA179845 | AA219691 | Hs 73625 | RAB6 interacting, kinesin-like (rabkines |
| 109456 | AA232645 | AW956580 | Hs.42699 | ESTs |
| 109768 | F10399 | F06838 | Hs.14763 | ESTs |
| 110107 | H16772 | AW151660 | Hs.31444 | ESTs |
| 110906 | N39584 | AA035211 | Hs.17404 | ESTs |
| 110984 | N52006 | AW613287 | Hs.80120 | UDP-N-acetyl-alpha-D-galactosamine: polyp |
| 111006 | N53375 | BE387014 | Hs.166146 | Horner, neuronal immediate early gene, 3 |
| 111018 | N54067 | AI287912 | Hs.3628 | mitogen-activated protein kinase kinase |
| 111133 | N64436 | AW580939 | Hs.97199 | complement component C1q receptor |
| 111760 | R26892 | BE551929 | Hs.268754 | Homo sapiens cDNA FLJ11949 fis, clone HE |
| 113073 | T33637 | N39342 | Hs.103042 | microtubule-associated protein 1B |
| 113195 | T57112 | H83265 | Hs.8881 | ESTs, Weakly similar to S41044 chromosom |
| 113923 | W80763 | AW953484 | Hs.3849 | hypothetical protein FLJ22041 similar to |
| 114521 | AA046808 | AW139036 | Hs.108957 | 40S ribosomal protein S27 isoform |
| 115061 | AA253217 | AI751438 | Hs.41271 | Homo sapiens mRNA full length insert cDN |
| 115096 | AA255991 | AI683069 | Hs.175319 | ESTs |
| 115145 | AA258138 | AA740907 | Hs.88297 | ESTs |
| 115819 | AA426573 | AA486620 | Hs 41135 | endomucin-2 |
| 115947 | AA443793 | R47479 | Hs.94761 | KIAA1691 protein |
| 116314 | AA490588 | AI799104 | Hs.178705 | Homo sapiens cDNA FLJ11333 fis, clone PL |
| 116339 | AA496257 | AK000290 | Hs.44033 | dipeptidyl peptidase 8 |
| 116430 | AA609717 | AK001531 | Hs.66048 | hypothetical protein FLJ10669 |
| 116589 | D59570 | AI557212 | Hs.17132 | ESTs, Moderately similar to I54374 gene |
| 116733 | F13787 | AL157424 | Hs.61289 | synaptojanin 2 |
| 117023 | H88157 | AW070211 | Hs.102415 | Homo sapiens mRNA; cDNA DKFZp586N0121 (f |
| 117186 | H98988 | H98988 | Hs.42612 | ESTs, Weakly similar to ALU1_HUMAN ALU S |
| 117563 | N34287 | AF055634 | Hs.44553 | unc5 (C. elegans homolog) c |
| 117997 | N52090 | N52090 | Hs.47420 | EST |
| 118475 | N66845 | N66845 | | gb:za46c11 s1 Soares fetal liver spleen |
| 118581 | N68905 | N68905 | | gb:za69b09 s1 Soares_fetal_lung_NbHL19W |
| 119073 | R32894 | BE245360 | Hs.279477 | ESTs |
| 119155 | R61715 | R61715 | Hs 310598 | ESTs, Moderately similar to ALU1_HUMAN A |
| 119174 | R71234 | R71234 | | gb:yi54c08.s1 Soares placenta Nb2HP Homo |
| 119221 | R98105 | C14322 | Hs.250700 | tryptase beta 1 |
| 119416 | T97186 | T97186 | | gb:ye50h09.s1 Soares fetal liver spleen |
| 119866 | W80814 | AA496205 | Hs.193700 | Homo sapiens mRNA; cDNA DKFZp586I0324 (f |
| 121335 | AA404418 | AA404418 | | gb:zw37e02.s1 Soares_total_fetus_Nb2HF8_{—} |
| 121381 | AA405747 | AW088642 | Hs.97984 | hypothetical protein FLJ22252 similar to |
| 123160 | AA488687 | AA488687 | Hs.284235 | ESTs, Weakly similar to I38022 hypotheti |
| 123473 | AA599143 | AA599143 | | gb:ae52d04.s1 Stratagene lung carcinoma |
| 123523 | AA608588 | AA608588 | | gb:ae54e06.s1 Stratagene lung carcinoma |
| 123533 | AA608751 | AA608751 | | gb:ae56h07.s1 Stratagene lung carcinoma |
| 123964 | C13961 | C13961 | | gb:C13961 Clontech human aorta polyA + mR |
| 124006 | D60302 | AI147155 | Hs.270016 | ESTs |
| 124315 | H94892 | NM_005402 | Hs.288757 | v-ral simian leukemia viral oncogene hom |
| 124659 | N93521 | AI680737 | Hs.289068 | Homo sapiens cDNA FLJ11918 fis, clone HE |
| 124669 | N95477 | AI571594 | Hs.102943 | hypothetical protein MGC12916 |
| 124847 | R60044 | W07701 | Hs.304177 | Homo sapiens clone FLB8503 PRO2286 mRNA, |
| 124875 | R70506 | AI887664 | Hs.285814 | sprouty (Drosophila) homolog 4 |
| 125091 | T91518 | T91518 | | gb:ye20f05 s1 Stratagene lung (937210) H |
| 125103 | T95333 | AA570056 | Hs.122730 | ESTs, Moderately similar to KIAA1215 pro |
| 125355 | R45630 | R60547 | Hs.170098 | KIAA0372 gene product |
| 125565 | R20839 | R20840 | | gb:yg05c08.r1 Soares infant brain 1NIB H |
| 125590 | R23858 | R23858 | Hs.143375 | Homo sapiens, clone IMAGE:3840937, mRNA, |
| 423765 | R23858 | R23858 | Hs.143375 | Homo sapiens, clone IMAGE:3840937, mRNA, |
| 126511 | AI024874 | T92143 | Hs.57958 | EGF-TM7-latrophilin-related protein |
| 100286 | W26247 | BE247550 | Hs 86859 | growth factor receptor-bound protein 7 |
| 126563 | W26247 | AA516391 | Hs.181368 | U5 snRNP-specific protein (220 kD), orth |
| 126649 | AA856990 | AA001860 | Hs.279531 | ESTs |
| 449602 | AA856990 | AA001860 | Hs.279531 | ESTs |
| 126872 | AA136653 | AW450979 | | gb:UI-H-BI3-ala-a-12-0-UI.s1 NCI_CGAP_Su |
| 456000 | AA136653 | BE180876 | Hs 11614 | HSPC065 protein |
| 414221 | AA136653 | AW450979 | | gb:UI-H-BI3-ala-a-12-0-UI.s1 NCI_CGAP_Su |
| 127402 | AA358869 | AA358869 | Hs.227949 | SEC13(S. cerevisiae)-like 1 |
| 127651 | AI123976 | AA382523 | Hs 105689 | MSTP031 protein |
| 424806 | AI123976 | AA382523 | Hs.105689 | MSTP031 protein |
| 128062 | AA379500 | AA379621 | Hs 105547 | neural proliferation, differentiation an |
| 128992 | R49693 | H04150 | Hs.107708 | ESTs |
| 129046 | AA195678 | AB029290 | Hs.108258 | actin binding protein; macrophin (microf |
| 129188 | M30257 | NM_001078 | Hs.109225 | vascular cell adhesion molecule 1 |
| 129314 | AA028131 | BE622768 | Hs 290356 | mesoderm development candidate 1 |
| 129371 | M10321 | X06828 | Hs.110802 | von Willebrand factor |
| 129468 | J03040 | AW410538 | Hs 111779 | secreted protein, acidic, cysteine-rich |
| 129765 | M86933 | M86933 | Hs.1238 | amelogenin (Y chromosome) |
| 129805 | AA012933 | AA012848 | Hs 12570 | tubulin-specific chaperone d |
| 129884 | AA286710 | AF055581 | Hs.13131 | lysosomal |
| 130495 | AA243278 | AW250380 | Hs.109059 | mitochondrial ribosomal protein L12 |
| 130639 | D59711 | AI557212 | Hs.17132 | ESTs, Moderately similar to I54374 gene |
| 130657 | T94452 | AW337575 | Hs.201591 | ESTs |
| 130828 | AA053400 | AW631469 | Hs.203213 | ESTs |
| 130972 | AA370302 | D81866 | Hs.21739 | Homo sapiens mRNA, cDNA DKFZp586I1518 (f |
| 131080 | J05008 | NM_001955 | Hs.2271 | endothelin 1 |
| 131137 | U85193 | W27392 | Hs.33287 | nuclear factor I/B |
| 131182 | AA256153 | AI824144 | Hs.23912 | ESTs |
| 131486 | X83107 | F06972 | Hs 27372 | BMX non-receptor tyrosine kinase |
| 131573 | AA046593 | AA040311 | Hs.28959 | ESTs |
| 131647 | AA410480 | AA359615 | Hs.30089 | ESTs |
| 131756 | D45304 | AA443966 | Hs.31595 | ESTs |
| 131859 | AA90657 | AW960564 | | transmembrane 4 superfamily member 1 |
| 131881 | AA010163 | AW361018 | Hs.3383 | upstream regulatory element binding prot |
| 132050 | AA136353 | AI267615 | Hs 38022 | ESTs |
| 132083 | Y07867 | BE386490 | Hs.279663 | Pirin |
| 132164 | U84573 | AI752235 | Hs.41270 | procollagen-lysine, 2-oxoglutarate 5-dio |
| 132358 | X60486 | NM_003542 | Hs.46423 | H4 histone family, member G |
| 132413 | AA132969 | AW361383 | Hs 260116 | metalloprotease 1 (pitrilysin family) |
| 132456 | AA114250 | AB011084 | Hs.48924 | KIAA0512 gene product; ALEX2 |
| 132490 | F13782 | NM_001290 | Hs.4980 | LIM domain binding 2 |
| 132676 | AA283035 | N92589 | Hs.261038 | ESTs, Weakly similar to I38022 hypotheti |
| 132687 | AB002301 | AB002301 | Hs.54985 | KIAA0303 protein |
| 132718 | AA056731 | NM_004600 | Hs 554 | Sjogren syndrome antigen A2 (60 kD, ribon |
| 132736 | U68019 | AW081883 | Hs.211578 | Homo sapiens cDNA: FLJ23037 fis, clone L |
| 132760 | H99198 | AA125985 | Hs.56145 | thymosin, beta, identified in neuroblast |
| 132933 | AA598702 | BE263252 | Hs.6101 | hypothetical protein MGC3178 |
| 132968 | N77151 | AF234532 | Hs 61638 | myosin X |
| 132994 | AA505133 | AA112748 | Hs.279905 | clone HQ0310 PRO0310p1 |
| 133061 | AB000584 | AI186431 | Hs.296638 | prostate differentiation factor |
| 133147 | D12763 | AA026533 | Hs.66 | interleukin 1 receptor-like 1 |
| 133161 | AA253193 | AW021103 | Hs.6631 | hypothetical protein FLJ20373 |
| 133200 | AA432248 | AB037715 | Hs.183639 | hypothetical protein FLJ10210 |
| 133260 | AA083572 | AA403045 | Hs.6906 | Homo sapiens cDNA: FLJ23197 fis, clone R |
| 133363 | AA479713 | AI866286 | Hs.71962 | ESTs, Weakly similar to B36298 proline-r |
| 133491 | L40395 | BE619053 | Hs.170001 | eukaryotic translation initiation factor |
| 133517 | X52947 | NM_000165 | Hs.74471 | gap junction protein, alpha 1, 43 kD (con |
| 133550 | W80846 | AI129903 | Hs.74669 | vesicle-associated membrane protein 5 (m |
| 133607 | M34539 | BE273749 | | FK506-binding protein 1A (12 kD) |
| 133614 | D67029 | NM_003003 | Hs.75232 | SEC14 (S. cerevisiae)-like 1 |
| 133627 | U09587 | NM_002047 | Hs.75280 | glycyl-tRNA synthetase |
| 133691 | M85289 | M85289 | Hs 211573 | heparan sulfate proteoglycan 2 (perlecan |
| 133696 | D10522 | AI878921 | Hs.75607 | myristoylated alanine-rich protein kinas |
| 133913 | W84712 | AU076964 | Hs.7753 | calumenin |
| 133975 | D29992 | C18356 | Hs.295944 | tissue factor pathway inhibitor 2 |
| 133985 | L34657 | L34657 | Hs.78146 | platelet/endothelial cell adhesion molec |
| 134039 | S78569 | NM_002290 | Hs.78672 | laminin, alpha 4 |
| 134088 | D43636 | AI379954 | Hs 79025 | KIAA0096 protein |
| 134161 | U97188 | AA634543 | Hs.79440 | IGF-II mRNA-binding protein 3 |
| 134299 | AA487558 | AW580939 | Hs 97199 | complement component C1q receptor |
| 134416 | M28882 | X68264 | Hs 211579 | melanoma cell adhesion molecule |
| 116470 | X70683 | AI272141 | Hs.83484 | SRY (sex determining region Y)-box 4 |
| 134656 | X14787 | AI750878 | Hs.87409 | thrombospondin 1 |
| 134989 | AA236324 | AW968058 | Hs.92381 | nudix (nucleoside diphosphate linked moi |
| 135051 | C15324 | AI272141 | Hs.83484 | SRY (sex determining region Y)-box 4 |
| 135073 | AA452000 | W55956 | Hs.94030 | Homo sapiens mRNA; cDNA DKFZp586E1624 (f |
| 135349 | D83174 | AA114212 | Hs 9930 | serine (or cysteine) proteinase inhibito |
| 100114 | D00596 | X02308 | Hs.82962 | thymidylate synthetase |
| 100130 | D11428 | NM_000304 | Hs.103724 | peripheral myelin protein 22 |
| 100143 | D13640 | AU076465 | Hs.278441 | KIAA0015 gene product |
| 100168 | D14874 | H73444 | Hs.394 | adrenomedullin |
| 100208 | D26129 | NM_002933 | Hs 78224 | ribonuclease, RNase A family, 1 (pancrea |
| 100224 | D28476 | AL121516 | Hs.138617 | thyroid hormone receptor interactor 12 |
| 100405 | D86425 | AW291587 | Hs.82733 | nidogen 2 |
| 100420 | D86983 | D86983 | Hs.118893 | Melanoma associated gene |
| 100455 | D87953 | AW888941 | Hs.75789 | N-myc downstream regulated |
| 100529 | HG1862-HT1897 | BE313693 | Hs.334330 | calmodulin 2 (phosphorylase kinase, delt |
| 100618 | HG2614-HT2710 | AI752163 | Hs 114599 | collagen, type VIII, alpha 1 |
| 100619 | HG2639-HT2735 | N24433 | Hs.241567 | RNA binding motif, single stranded inter |
| 100658 | HG2855-HT2995 | U56725 | Hs.180414 | heat shock 70 kD protein 2 |
| 100676 | HG3044-HT3742 | X02761 | Hs.287820 | fibronectin 1 |
| 100718 | HG3342-HT3519 | BE295928 | Hs.75424 | inhibitor of DNA binding 1, dominant neg |
| 100752 | HG3543-HT3739 | T81309 | | insulin-like growth factor 2 (somatomedi |
| 100828 | HG4069-HT4339 | AL048753 | Hs.303649 | small inducible cytokine A2 (monocyte ch |
| 100850 | HG417-HT417 | AA836472 | Hs 297939 | cathepsin B |
| 100991 | J03764 | J03836 | Hs.82085 | serine (or cysteine) proteinase inhibito |
| 101097 | L06797 | BE245301 | Hs.89414 | chemokine (C—X—C motif), receptor 4 (fus |
| 101110 | L08246 | AI439011 | Hs.86386 | myeloid cell leukemia sequence 1 (BCL2-r |
| 101142 | L12711 | L12711 | Hs.89643 | transketolase (Wernicke-Korsakoff syndro |
| 101156 | L13977 | AA340987 | Hs.75693 | prolylcarboxypeptidase (angiotensinase C |
| 101168 | L15388 | NM_005308 | Hs.211569 | G protein-coupled receptor kinase 5 |
| 101184 | L19871 | NM_001674 | Hs.460 | activating transcription factor 3 |
| 101192 | L20859 | BE247295 | Hs 78452 | solute carrier family 20 (phosphate tran |
| 101317 | L42176 | L42176 | Hs.8302 | four and a half LIM domains 2 |
| 101336 | L49169 | NM_006732 | Hs 75678 | FBJ murine osteosarcoma viral oncogene h |
| 101345 | L76380 | NM_005795 | Hs.152175 | calcitonin receptor-like |
| 101400 | M15990 | M15990 | Hs.194148 | v-yes-1 Yamaguchi sarcoma viral oncogene |
| 101475 | M23254 | BE410405 | Hs.76288 | calpain 2, (m/ll) large subunit |
| 101485 | M24736 | AA296520 | Hs.89546 | selectin E (endothelial adhesion molecul |
| 101496 | M26576 | X12784 | Hs.119129 | collagen, type IV, alpha 1 |
| 101505 | M27396 | AA307680 | Hs 75692 | asparagine synthetase |
| 101543 | M31166 | M31166 | Hs.2050 | pentaxin-related gene, rapidly induced b |
| 101557 | M31994 | BE293116 | Hs.76392 | aldehyde dehydrogenase 1 family, member |
| 101560 | M32334 | AW958272 | Hs 347326 | intercellular adhesion molecule 2 |
| 101587 | M35878 | AI752416 | Hs 77326 | insulin-like growth factor binding prote |
| 101592 | M36429 | AF064853 | Hs.91299 | guanine nucleotide binding protein (G pr |
| 101633 | M57730 | NM_004428 | Hs.1624 | ephrin-A1 |
| 101634 | M57731 | AV650262 | Hs 75765 | GRO2 oncogene |
| 101667 | M60858 | NM_005381 | | nucleolin |
| 101682 | M62994 | AF043045 | Hs.81008 | filamin B, beta (actin-binding protein-2 |
| 101714 | M68874 | M68874 | Hs.211587 | phospholipase A2, group IVA (cytosolic, |
| 101720 | M69043 | M69043 | Hs.81328 | nuclear factor of kappa light polypeptid |
| 101741 | M74719 | NM_003199 | Hs.326198 | transcription factor 4 |
| 101744 | M75126 | AI879352 | Hs.118625 | hexokinase 1 |
| 101793 | M84349 | W01076 | Hs.278573 | CD59 antigen p18-20 (antigen identified |
| 101837 | M92843 | M92843 | Hs.343586 | zinc finger protein homologous to Zfp-36 |
| 101838 | M92934 | BE243845 | Hs.75511 | connective tissue growth factor |
| 101840 | M93056 | AA236291 | Hs.183583 | serine (or cysteine) proteinase inhibito |
| 101857 | M94856 | BE550723 | Hs 153179 | fatty acid binding protein 5 (psoriasis- |
| 101864 | M95787 | BE392588 | Hs.75777 | transgelin |
| 101931 | S76965 | NM_006823 | Hs.75209 | protein kinase (cAMP-dependent, catalyti |
| 101966 | S81914 | X96438 | Hs.76095 | immediate early response 3 |
| 102012 | U03057 | BE259035 | Hs.118400 | singed (Drosophila)-like (sea urchin fas |
| 102013 | U03100 | BE616287 | Hs.178452 | catenin (cadherin-associated protein), a |
| 102024 | U03877 | AA301867 | Hs.76224 | EGF-containing fibulin-like extracellula |
| 102059 | U08021 | AI752666 | Hs.76669 | nicotinamide N-methyltransferase |
| 102121 | U14391 | NM_004998 | Hs.82251 | myosin IE |
| 102283 | U31384 | AW161552 | Hs 83381 | guanine nucleotide binding protein 11 |
| 102300 | U32944 | AI929721 | Hs.5120 | dynein, cytoplasmic, light polypeptide |
| 102378 | U40369 | AU076887 | Hs.28491 | spermidine/spermine N1-acetyltransferase |
| 102395 | U41767 | AU077005 | Hs.92208 | a disintegrin and metalloproteinase doma |
| 102460 | U48959 | U48959 | Hs.211582 | myosin, light polypeptide kinase |
| 102491 | U51010 | U51010 | | gb:Human nicotinamide N-methyltransferas |
| 102499 | U51478 | BE243877 | Hs.76941 | ATPase, Na+/K+ transporting, beta 3 poly |
| 102523 | U53445 | U53445 | Hs 15432 | downregulated in ovarian cancer 1 |
| 102560 | U59289 | R97457 | Hs.63984 | cadherin 13, H-cadherin (heart) |
| 102564 | U59423 | U59423 | Hs.79067 | MAD (mothers against decapentaplegic, Dr |
| 102589 | U62015 | AU076728 | Hs.8867 | cysteine-rich, angiogenic inducer, 61 |
| 102600 | U63825 | AI984144 | Hs.66713 | hepatitis delta antigen-interacting prot |
| 102645 | U67963 | AL119566 | Hs 6721 | lysosomal |
| 102687 | U73379 | NM_007019 | Hs.93002 | ubiquitin carrier protein E2-C |
| 102693 | U73824 | AA532780 | Hs.183684 | eukaryotic translation initiation factor |
| 102709 | U77604 | AA122237 | Hs.81874 | microsomal glutathione S-transferase 2 |
| 102759 | U81607 | NM_005100 | Hs.788 | A kinase (PRKA) anchor protein (gravin) |
| 102804 | U89942 | NM_002318 | Hs.83354 | lysyl oxidase-like 2 |
| 102882 | X04412 | AI767736 | Hs 290070 | gelsolin (amyloidosis, Finnish type) |
| 102907 | X06985 | BE409861 | Hs.202833 | heme oxygenase (decycling) 1 |
| 102915 | X07820 | X07820 | Hs 2258 | matrix metalloproteinase 10 (stromelysin |
| 102927 | X12876 | BE512730 | Hs.65114 | keratin 18 |
| 102960 | X15729 | AI904738 | Hs.76053 | DEAD/H (Asp-Glu-Ala-Asp/His) box polypep |
| 103011 | X52541 | AJ243425 | Hs.326035 | early growth response 1 |
| 103020 | X53416 | X53416 | Hs.195464 | filamin A, alpha (actin-binding protein- |
| 103029 | X54489 | AW800726 | Hs.789 | GRO1 oncogene (melanoma growth stimulati |
| 103036 | X54925 | M13509 | Hs.83169 | matrix metalloproteinase 1 (interstitial |
| 103056 | X57206 | Y18024 | Hs.78877 | inositol 1,4,5-trisphosphate 3-kinase B |
| 103080 | X59798 | AU077231 | Hs.82932 | cyclin D1 (PRAD1: parathyroid adenomatos |
| 103095 | X60957 | NM_005424 | Hs.78824 | tyrosine kinase with immunoglobulin and |
| 103138 | X65965 | X65965 | | gb:H. sapiens SOD-2 gene for manganese su |
| 103176 | X69111 | AL021154 | Hs.76884 | inhibitor of DNA binding 3, dominant neg |
| 103195 | X70940 | AA351647 | Hs.2642 | eukaryotic translation elongation factor |
| 103347 | X87838 | AU077309 | Hs.171271 | catenin (cadherin-associated protein), b |
| 103371 | X91247 | X91247 | Hs 13046 | thioredoxin reductase 1 |
| 103432 | X97748 | X97748 | | gb:H. sapiens PTX3 gene promotor region. |
| 103471 | Y00815 | Y00815 | Hs.75216 | protein tyrosine phosphatase, receptor t |
| 103967 | AA303711 | AL120051 | Hs 144700 | ephrin-B1 |
| 104447 | L44538 | AW204145 | Hs.156044 | ESTs |
| 104764 | AA025351 | AI039243 | Hs.278585 | ESTs |
| 104783 | AA027050 | AA533513 | Hs.93659 | protein disulfide isomerase related prot |
| 104798 | AA029462 | AW952619 | Hs.17235 | Homo sapiens clone TCCCIA00176 mRNA sequ |
| 104865 | AA045136 | T79340 | Hs.22575 | B-cell CLL/lymphoma 6, member B (zinc fi |
| 104877 | AA047437 | AL138635 | Hs.22968 | Homo sapiens clone IMAGE:451939, mRNA se |
| 104894 | AA054087 | AF065214 | Hs.18858 | phospholipase A2, group IVC (cytosolic, |
| 104952 | AA071089 | AW076098 | Hs.345588 | desmoplakin (DPI, DPII) |
| 105113 | AA156450 | AB037816 | Hs 8982 | Homo sapiens, clone IMAGE: 3506202, mRNA, |
| 105178 | AA187490 | AA313825 | Hs 21941 | AD036 protein |
| 105196 | AA195031 | W84893 | Hs.9305 | angiotensin receptor-like 1 |
| 105215 | AA205724 | AA205759 | Hs.10119 | hypothetical protein FLJ14957 |
| 105263 | AA227926 | AW388633 | Hs.6682 | solute carrier family 7, (cationic amino |
| 105271 | AA227986 | AA807881 | Hs.25329 | ESTs |
| 105330 | AA234743 | AW338625 | Hs.22120 | ESTs |
| 105461 | AA253216 | BE539071 | Hs.69388 | hypothetical protein FLJ20505 |
| 105492 | AA256210 | AI805717 | Hs.289112 | CGI-43 protein |
| 105493 | AA256268 | AL047586 | Hs.10283 | RNA binding motif protein 8B |
| 105594 | AA279397 | AB024334 | Hs.25001 | tyrosine 3-monooxygenase/tryptophan 5-mo |
| 105727 | AA292379 | AL135159 | Hs.20340 | KIAA1002 protein |
| 105732 | AA292717 | AW504170 | Hs.274344 | hypothetical protein MGC12942 |
| 105767 | AA346551 | AW370946 | Hs.23457 | ESTs |
| 105882 | AA400292 | W46802 | Hs 81988 | disabled (Drosophila) homolog 2 (mitogen |
| 105936 | AA404338 | AI678765 | Hs.21812 | ESTs |
| 106031 | AA412284 | X64116 | Hs.171844 | Homo sapiens cDNA: FLJ22296 fis, clone H |
| 106124 | AA423987 | H93366 | Hs 7567 | Homo sapiens cDNA: FLJ21962 fis, clone H |
| 106222 | AA428594 | AA356392 | Hs.21321 | Homo sapiens clone FLB9213 PRO2474 mRNA, |
| 106241 | AA430108 | BE019681 | Hs.6019 | Homo sapiens cDNA: FLJ21288 fis, clone C |
| 106263 | AA431462 | W21493 | Hs 28329 | hypothetical protein FLJ14005 |
| 106264 | AA431470 | AL046859 | Hs.3407 | protein kinase (cAMP-dependent, catalyti |
| 106366 | AA443756 | AA186715 | Hs 336429 | RIKEN cDNA 9130422N19 gene |
| 106454 | AA449479 | NM_014038 | Hs 5216 | HSPC028 protein |
| 106634 | AA459916 | W25491 | Hs 288909 | hypothetical protein FLJ22471 |
| 106724 | AA465226 | N48670 | Hs.28631 | Homo sapiens cDNA: FLJ22141 fis, clone H |
| 106793 | AA478778 | H94997 | Hs.16450 | ESTs |
| 106799 | AA479037 | BE313412 | Hs.7961 | Homo sapiens clone 25012 mRNA sequence |
| 106842 | AA482597 | AF124251 | Hs.26054 | novel SH2-containing protein 3 |
| 106868 | AA487561 | BE185536 | Hs 301183 | molecule possessing ankynn repeats indu |
| 106890 | AA489245 | AA489245 | Hs.88500 | mitogen-activated protein kinase 8 inter |
| 106961 | AA504110 | AW243614 | Hs.18063 | Homo sapiens cDNA FLJ10768 fis, clone NT |
| 106974 | AA520989 | AI817130 | Hs 9195 | Homo sapiens cDNA FLJ13698 fis, clone PL |
| 107030 | AA599434 | AL117424 | Hs.25035 | chloride intracellular channel 4 |
| 107061 | AA608649 | BE147611 | Hs 6354 | stromal cell derived factor receptor 1 |
| 107086 | AA609519 | NM_012331 | Hs 26458 | methionine sulfoxide reductase A |
| 107216 | D51069 | D51069 | Hs.211579 | melanoma cell adhesion molecule |
| 107385 | U97519 | NM_005397 | Hs.16426 | podocalyxin-like |
| 107444 | W28391 | W28391 | Hs 343258 | proliferation-associated 2G4, 38 kD |
| 107985 | AA035638 | T40064 | Hs.71968 | Homo sapiens mRNA; cDNA DKFZp564F053 (fr |
| 108507 | AA083514 | AI554545 | Hs.68301 | ESTs |
| 108695 | AA121315 | AB029000 | Hs.70823 | KIAA1077 protein |
| 108931 | AA147186 | AA147186 | | gb:zo38d01.s1 Stratagene endothelial cel |
| 109001 | AA156125 | AI056548 | Hs 72116 | hypothetical protein FLJ20992 similar to |
| 109195 | AA188932 | AF047033 | Hs.132904 | solute carrier family 4, sodium bicarbon |
| 109390 | AA219653 | AW007485 | Hs.87125 | EH-domain containing 3 |
| 109456 | AA232645 | AW956580 | Hs.42699 | ESTs |
| 109737 | F10078 | AA055415 | Hs.13233 | ESTs, Moderately similar to A47582 B-cel |
| 110411 | H48032 | AW001579 | Hs.9645 | Homo sapiens mRNA for KIAA1741 protein, |
| 110660 | H82117 | AA782114 | Hs 28043 | ESTs |
| 110906 | N39584 | AA035211 | Hs.17404 | ESTs |
| 111018 | N54067 | AI287912 | Hs.3628 | mitogen-activated protein kinase kinase |
| 111091 | N59858 | AA300067 | Hs.33032 | hypothetical protein DKFZp434N185 |
| 111356 | N90933 | BE301871 | Hs.4867 | mannosyl (alpha-1,3-)-glycoprotein beta- |
| 111378 | N93764 | AW160993 | Hs.326292 | hypothetical gene DKFZp434A1114 |
| 111741 | R26124 | AB020653 | Hs.24024 | KIAA0846 protein |
| 111769 | R27957 | AW629414 | Hs.24230 | ESTs |
| 112318 | R55470 | AW083384 | Hs.11067 | ESTs, Highly similar to T46395 hypotheti |
| 112951 | T16550 | AA307634 | Hs.6650 | vacuolar protein sorting 45B (yeast homo |
| 113057 | T26674 | AW194301 | Hs.339283 | Human DMA sequence from clone RP1-187J11 |
| 113195 | T57112 | H83265 | Hs.8881 | ESTs, Weakly similar to S41044 chromosom |
| 113490 | T88700 | BE178110 | Hs.173374 | Homo sapiens cDNA FLJ10500 fis, clone NT |
| 113542 | T90527 | H43374 | Hs.7890 | Homo sapiens mRNA for KIAA1671 protein, |
| 113803 | W42789 | AW880709 | Hs.283683 | chromosome 8 open reading frame 4 |
| 113847 | W60002 | NM_005032 | Hs.4114 | plastin 3 (T isoform) |
| 113910 | W78175 | AA113262 | Hs.17901 | Homo sapiens, clone IMAGE:3937015, mRNA, |
| 113947 | W84768 | W84768 | | gb:zh53d03.s1 Soares_fetal_liver_spleen_{—} |
| 114047 | W94427 | AL035858 | Hs.3807 | FXYD domain-containing ion transport reg |
| 115061 | AA253217 | AI751438 | Hs.41271 | Homo sapiens mRNA full length insert cDN |
| 115819 | AA426573 | AA486620 | Hs.41135 | endomucin-2 |
| 115870 | AA432374 | NM_005985 | Hs 48029 | snail 1 (drosophila homolog), zinc finge |
| 115964 | AA446622 | AA987568 | Hs.74313 | KIAA1265 protein |
| 116228 | AA478771 | AI767947 | Hs 50841 | ESTs |
| 116264 | AA482594 | D51174 | Hs 272239 | lysosomal |
| 116314 | AA490588 | AI799104 | Hs.178705 | Homo sapiens cDNA FLJ11333 fis, clone PL |
| 116589 | D59570 | AI557212 | Hs 17132 | ESTs, Moderately similar to I54374 gene |
| 117023 | H88157 | AW070211 | Hs.102415 | Homo sapiens mRNA; cDNA DKFZp586N0121 (f |
| 117112 | H94648 | AW969999 | Hs.293658 | ESTs |
| 117156 | H97538 | W73853 | | ESTs |
| 117176 | H98670 | H45100 | Hs.49753 | uveal autoantigen with coiled coil domai |
| 117280 | N22107 | M18217 | Hs.172129 | Homo sapiens cDNA: FLJ21409 fis, clone C |
| 119559 | W38197 | W38197 | | Empirically selected from AFFX single pr |
| 119866 | W80814 | AA496205 | Hs.193700 | Homo sapiens mRNA; cDNA DKFZp586I0324 (f |
| 120655 | AA287347 | AA305599 | Hs.238205 | hypothetical protein PRO2013 |
| 121314 | AA402799 | W07343 | Hs.182538 | phospholipid scramblase 4 |
| 121335 | AA404418 | AA404418 | | gb:zw37e02.s1 Soares_total_fetus_Nb2HF8_{—} |
| 121822 | AA425107 | AI743860 | | metallothionein 1E (functional) |
| 121835 | AA425435 | AB033030 | Hs.300670 | KIAA1204 protein |
| 122331 | AA442872 | AL133437 | Hs.110771 | Homo sapiens cDNA: FLJ21904 fis, clone H |
| 122577 | AA452860 | AA829725 | Hs.334437 | hypothetical protein MGC4248 |
| 123160 | AA488687 | AA488687 | Hs 284235 | ESTs, Weakly similar to I38022 hypotheti |
| 123486 | AA599674 | BE019072 | Hs.334802 | Homo sapiens cDNA FLJ14680 fis, clone NT |
| 124059 | F13673 | BE387335 | Hs.283713 | ESTs, Weakly similar to S64054 hypotheti |
| 124339 | H99093 | H99093 | Hs.343411 | DEAD/H (Asp-Glu-Ala-Asp/His) box polypep |
| 124358 | N22495 | AW070211 | Hs.102415 | Homo sapiens mRNA, cDNA DKFZp586N0121 (f |
| 124364 | N23031 | AF265555 | Hs 250646 | baculoviral IAP repeat-containing 6 |
| 124726 | R15740 | NM_003654 | Hs.104576 | carbohydrate (keratan sulfate Gal-6) sul |
| 124763 | R39610 | BE410405 | Hs 76288 | calpain 2, (m/ll) large subunit |
| 125167 | W45560 | AL137540 | Hs.102541 | netrin 4 |
| 125304 | Z39833 | AL359573 | Hs 124940 | GTP-binding protein |
| 125307 | Z40583 | AW580945 | Hs.330466 | ESTs |
| 125329 | AA825437 | AA825437 | Hs 58875 | ESTs |
| 107985 | R66613 | T40064 | Hs.71968 | Homo sapiens mRNA; cDNA DKFZp564F053 (fr |
| 125598 | R66613 | T40064 | Hs.71968 | Homo sapiens mRNA; cDNA DKFZp564F053 (fr |
| 125609 | AA868063 | AA868063 | Hs.104576 | carbohydrate (keratan sulfate Gal-6) sul |
| 116024 | AA128075 | AA088767 | Hs.83883 | transmembrane, prostate androgen induced |
| 418000 | AA128075 | AA932794 | Hs.83147 | guanine nucleotide binding protein-like |
| 126399 | AA128075 | AA088767 | Hs 83883 | transmembrane, prostate androgen induced |
| 127435 | N66570 | X69086 | Hs.286161 | Homo sapiens cDNA FLJ13613 fis, clone PL |
| 127566 | AI051390 | AI051390 | Hs.116731 | ESTs |
| 127619 | AA627122 | AA627122 | Hs 163787 | ESTs |
| 434190 | AA627122 | AA627122 | Hs 163787 | ESTs |
| 128453 | X02761 | X02761 | Hs.287820 | fibronectin 1 |
| 128495 | AF010193 | NM_005904 | Hs 100602 | MAD (mothers against decapentaplegic, Dr |
| 128515 | AA149044 | BE395085 | Hs.10086 | type I transmembrane protein Fn14 |
| 128580 | U82108 | U82108 | Hs.101813 | solute carrier family 9 (sodium/hydrogen |
| 128623 | D78676 | BE076608 | Hs.105509 | CTL2 gene |
| 128642 | L35240 | Z28913 | Hs.102948 | enigma (LIM domain protein) |
| 128669 | AA598737 | W28493 | Hs.180414 | heat shock 70 kD protein 8 |
| 128903 | R69417 | AW150717 | Hs.345728 | STAT induced STAT inhibitor 3 |
| 128914 | AA232837 | AW867491 | Hs.107125 | plasmalemma vesicle associated protein |
| 129087 | N72695 | AI348027 | Hs.108557 | hypothetical protein PP1057 |
| 129188 | M30257 | NM_001078 | Hs.109225 | vascular cell adhesion molecule 1 |
| 129226 | M96843 | BE222494 | Hs.180919 | inhibitor of DNA binding 2, dominant neg |
| 129265 | X68277 | AA530892 | Hs.171695 | dual specificity phosphatase 1 |
| 129345 | AA292440 | R22497 | Hs.110571 | growth arrest and DNA-damage-inducible, |
| 129468 | J03040 | AW410538 | Hs.111779 | secreted protein, acidic, cysteine-rich |
| 129488 | AA228107 | AW966728 | Hs.54642 | methionine adenosyltransferase II, beta |
| 101838 | AA449789 | BE243845 | Hs.75511 | connective tissue growth factor |
| 413731 | AA449789 | BE243845 | Hs.75511 | connective tissue growth factor |
| 129557 | W01367 | AL045404 | Hs.46366 | KIAA0948 protein |
| 129619 | AA610116 | AA209534 | Hs.284243 | tetraspan NET-6 protein |
| 129627 | AA258308 | T40064 | Hs.71968 | Homo sapiens mRNA; cDNA DKFZp564F053 (fr |
| 129762 | AA460273 | AA453694 | Hs.12372 | tripartite motif protein TRIM2 |
| 129884 | AA286710 | AF055581 | Hs.13131 | lysosomal |
| 130018 | T68873 | AA353093 | | metallothionein 1L |
| 130147 | D63476 | D63476 | Hs.172813 | PAK-interacting exchange factor beta |
| 130178 | M62403 | U20982 | Hs.1516 | insulin-like growth factor-binding prate |
| 130282 | X55740 | BE245380 | Hs.153952 | 5′ nucleotidase (CD73) |
| 130431 | L10284 | AW505214 | Hs.155560 | calnexin |
| 130495 | AA243278 | AW250380 | Hs.109059 | mitochondrial ribosomal protein L12 |
| 130553 | AA430032 | AF062649 | Hs 252587 | pituitary tumor-transforming 1 |
| 130638 | H16402 | AW021276 | Hs.17121 | ESTs |
| 130639 | D59711 | AI557212 | Hs.17132 | ESTs, Moderately similar to I54374 gene |
| 130657 | T94452 | AW337575 | Hs.201591 | ESTs |
| 130686 | AA431571 | BE548267 | Hs.337986 | Homo sapiens cDNA FLJ10934 fis, clone OV |
| 130776 | R79356 | AF167706 | Hs.19280 | cysteine-rich motor neuron 1 |
| 130818 | AA280375 | AW190920 | Hs 19928 | hypothetical protein SP329 |
| 130840 | Z49269 | BE048821 | Hs.20144 | small inducible cytokine subfamily A (Cy |
| 130899 | Z41740 | AI077288 | Hs.296323 | serum/glucocorticoid regulated kinase |
| 131002 | AA121543 | AL050295 | Hs 22039 | KIAA0758 protein |
| 131080 | J05008 | NM_001955 | Hs.2271 | endothelin 1 |
| 131084 | AA101878 | NM_017413 | Hs.303084 | apelin; peptide ligand for APJ receptor |
| 131091 | T35341 | AJ271216 | Hs.22880 | dipeptidylpeptidase III |
| 131107 | N87590 | BE620886 | Hs 75354 | GCN1 (general control of amino-acid synt |
| 131182 | AA256153 | AI824144 | Hs.23912 | ESTs |
| 131207 | W74533 | AF104266 | Hs.24212 | latrophilin |
| 131319 | U25997 | NM_003155 | Hs.25590 | stanniocalcin 1 |
| 131328 | V01512 | AW939251 | Hs.25647 | v-fos FBJ murine osteosarcoma viral onco |
| 131509 | X56681 | X56681 | Hs.2780 | jun D proto-oncogene |
| 131555 | AA161292 | T47364 | Hs.278613 | interferon, alpha-inducible protein 27 |
| 131564 | AA491465 | T93500 | Hs 28792 | Homo sapiens cDNA FLJ11041 fis, clone PL |
| 131573 | AA046593 | AA040311 | Hs 28959 | ESTs |
| 131692 | D50914 | BE559681 | Hs.30736 | KIAA0124 protein |
| 131756 | D45304 | AA443966 | Hs 31595 | ESTs |
| 131859 | M90657 | AW960564 | | transmembrane 4 superfamily member 1 |
| 131909 | W69127 | NM_016558 | Hs.274411 | SCAN domain-containing 1 |
| 131915 | AA316186 | AI161383 | Hs.34549 | ESTs, Highly similar to S945411 clone 4 |
| 132046 | AA384503 | AI359214 | Hs.179260 | chromosome 14 open reading frame 4 |
| 132050 | AA136353 | AI267615 | Hs.38022 | ESTs |
| 132151 | AA044755 | BE379499 | Hs.173705 | Homo sapiens cDNA: FLJ22050 fis, clone H |
| 132164 | U84573 | AI752235 | Hs.41270 | procollagen-lysine, 2-oxoglutarate 5-dio |
| 132187 | AA058911 | AA235709 | Hs.4193 | DKFZP586O1624 protein |
| 132303 | AA620962 | BE177330 | Hs.325093 | Homo sapiens cDNA: FLJ21210 fis, clone C |
| 132314 | AA285290 | AF112222 | Hs.323806 | pinin, desmosome associated protein |
| 132358 | X60486 | NM_003542 | Hs.46423 | H4 histone family, member G |
| 132398 | R31641 | AA876616 | Hs.16979 | ESTs, Weakly similar to A43932 mucin 2 p |
| 132421 | AA489190 | AW163483 | Hs.48320 | double ring-finger protein, Dorfin |
| 132490 | F13782 | NM_001290 | Hs.4980 | LIM domain binding 2 |
| 132520 | AA257993 | AA257992 | Hs 50651 | Janus kinase 1 (a protein tyrosine kinas |
| 132546 | M24283 | M24283 | Hs.168383 | intercellular adhesion molecule 1 (CD54) |
| 132610 | AA443114 | AA160511 | Hs.5326 | amino acid system N transporter 2; porcu |
| 132716 | T35289 | BE379595 | Hs 283738 | casein kinase 1, alpha 1 |
| 132840 | N23817 | BE218319 | Hs.5807 | GTPase Rab14 |
| 132883 | AA047151 | AA373314 | Hs 5897 | Homo sapiens mRNA; cDNA DKFZp586P1622 (f |
| 132968 | N77151 | AF234532 | Hs.61638 | myosin X |
| 132989 | AA480074 | AA480074 | Hs.331328 | hypothetical protein FLJ13213 |
| 132999 | Y00787 | Y00787 | Hs.624 | interleukin 8 |
| 133071 | T99789 | BE384932 | Hs 64313 | ESTs, Weakly similar to AF257182 1 G-pro |
| 133076 | W84341 | AW946276 | Hs.6441 | Homo sapiens mRNA; cDNA DKFZp586J021 (fr |
| 133099 | L09209 | W16518 | Hs.279518 | amyloid beta (A4) precursor-like protein |
| 133147 | D12763 | AA026533 | Hs 66 | interleukin 1 receptor-like 1 |
| 133149 | T16484 | AA370045 | Hs.6607 | AXIN1 up-regulated |
| 133161 | AA253193 | AW021103 | Hs 6631 | hypothetical protein FLJ20373 |
| 133200 | AA432248 | AB037715 | Hs.183639 | hypothetical protein FLJ10210 |
| 133220 | X82200 | NM_006074 | Hs.318501 | Homo sapiens mRNA full length insert cDN |
| 133260 | AA083572 | AA403045 | Hs.6906 | Homo sapiens cDNA: FLJ23197 fis, clone R |
| 133295 | L00352 | AI147861 | Hs.213289 | low density lipoprotein receptor (famili |
| 133349 | N75791 | AW631255 | Hs.8110 | L-3-hydroxyacyl-Coenzyme A dehydrogenase |
| 133391 | X57579 | AW103364 | Hs.727 | inhibin, beta A (activin A, activin AB a |
| 133398 | X02612 | NM_000499 | Hs.72912 | cytochrome P450, subfamily I (aromatic c |
| 133436 | H44631 | BE294068 | Hs.737 | immediate early protein |
| 133454 | AA090257 | BE547647 | Hs 177781 | hypothetical protein MGC5618 |
| 133478 | X83703 | X83703 | Hs.31432 | cardiac ankyrin repeat protein |
| 133491 | L40395 | BE619053 | Hs.170001 | eukaryotic translation initiation factor |
| 133510 | AA227913 | AW880841 | Hs.96908 | p53-induced protein |
| 133517 | X52947 | NM_000165 | Hs.74471 | gap junction protein, alpha 1, 43 kD (con |
| 133526 | M11313 | AU077051 | Hs.74561 | alpha-2-macroglobulin |
| 133538 | L14837 | NM_003257 | Hs.74614 | tight junction protein 1 (zona occludens |
| 133562 | M60721 | M60721 | Hs.74870 | H2.0 (Drosophila)-like homeo box 1 |
| 133584 | D90209 | D90209 | Hs.181243 | activating transcription factor 4 (tax-r |
| 133590 | T67986 | T70956 | Hs.75106 | clusterin (complement lysis inhibitor, S |
| 133617 | AA148318 | BE244334 | Hs.75249 | ADP-ribosylation factor-like 6 interact |
| 133651 | U97105 | AI301740 | Hs.173381 | dihydropyrimidinase-like 2 |
| 133671 | T25747 | AW503116 | Hs.301819 | zinc finger protein 146 |
| 133678 | K02574 | AW247252 | | nucleoside phosphorylase |
| 133681 | D78577 | AI352558 | | tyrosine 3-monooxygenase/tryptophan 5-mo |
| 133722 | X53331 | AW969976 | Hs.279009 | matrix Gla protein |
| 133730 | S73591 | BE242779 | Hs 179526 | upregulated by 1,25-dihydroxyvitamin D-3 |
| 133750 | X95735 | BE410769 | Hs.75873 | zyxin |
| 133802 | L16862 | AW239400 | Hs 76297 | G protein-coupled receptor kinase 6 |
| 133825 | U44975 | BE616902 | Hs.285313 | core promoter element binding protein |
| 133838 | M97796 | BE222494 | Hs.180919 | inhibitor of DNA binding 2, dominant neg |
| 133859 | U86782 | U86782 | Hs.178761 | 26S proteasome-associated pad1 homolog |
| 133889 | AA099391 | U48959 | Hs 211582 | myosin, light polypeptide kinase |
| 133960 | M19267 | M19267 | Hs.77899 | tropomyosin 1 (alpha) |
| 133975 | D29992 | C18356 | Hs.295944 | tissue factor pathway inhibitor 2 |
| 133977 | L19314 | AI125639 | Hs.250666 | hairy (Drosophila)-homolog |
| 134039 | S78569 | NM_002290 | Hs.78672 | laminin, alpha 4 |
| 134075 | U28811 | NM_012201 | Hs.78979 | Golgi apparatus protein 1 |
| 134081 | L77886 | AL034349 | Hs.79005 | protein tyrosine phosphatase, receptor t |
| 134164 | C14407 | AW245540 | Hs.79516 | brain abundant, membrane attached signal |
| 134203 | M60278 | AA161219 | Hs.799 | diphtheria toxin receptor (heparin-bindi |
| 134238 | R81509 | AA102179 | Hs.160726 | Homo sapiens cDNA FLJ11680 fis, clone HE |
| 134299 | AA487558 | AW580939 | Hs.97199 | complement component C1q receptor |
| 134332 | D86962 | D86962 | Hs.81875 | growth factor receptor-bound protein 10 |
| 134339 | AA478971 | R70429 | Hs.81988 | disabled (Drosophila) homolog 2 (mitogen |
| 134343 | D50683 | D50683 | Hs 82028 | transforming growth factor, beta recepto |
| 134381 | U56637 | AI557280 | Hs.184270 | capping protein (actin filament) muscle |
| 134403 | M61199 | AA334551 | | sperm specific antigen 2 |
| 134416 | M28882 | X68264 | Hs.211579 | melanoma cell adhesion molecule |
| 134493 | X15183 | M30627 | Hs.289088 | heat shock 90 kD protein 1, alpha |
| 134558 | S53911 | NM_001773 | Hs.85289 | CD34 antigen |
| 134817 | U20734 | AU076592 | Hs.198951 | jun B proto-oncogene |
| 134983 | D28235 | D28235 | Hs.196384 | prostaglandin-endoperoxide synthase 2 (p |
| 134989 | AA236324 | AW968058 | Hs 92381 | nudix (nucleoside diphosphate linked moi |
| 135052 | AA148923 | AL136653 | Hs.93675 | decidual protein induced by progesterone |
| 135062 | AA174183 | AK000967 | Hs 93872 | KIAA1682 protein |
| 135069 | AA456311 | AA876372 | Hs.93961 | Homo sapiens mRNA, cDNA DKFZp667D095 (fr |
| 135071 | L08069 | W27190 | Hs.94 | DnaJ (Hsp40) homolog, subfamily A, membe |
| 135073 | AA452000 | W55956 | Hs.94030 | Homo sapiens mRNA; cDNA DKFZp586E1624 (f |
| 135170 | AA282140 | T53169 | Hs.9587 | Homo sapiens cDNA: FLJ22290 fis, clone H |
| 135196 | J02854 | C03577 | Hs.9615 | myosin regulatory light chain 2, smooth |
| 135348 | AA442054 | U80983 | Hs 268177 | phospholipase C, gamma 1 (formerly subty |

Pkey: Unique Eos probeset identifier number
Accession: Accession number used for previous patent filings
ExAccn: Exemplar Accession number, Genbank accession number
UnigeneID: Unigene number
Unigene Title: Unigene gene title

TABLE 4A

| Pkey | CAT Number | Accession |
|---|---|---|
| 100752 | 33207_21 | T81309 BE019033 R94181 BE019198 NM_000612 J03242 AW411299 BE300064 |
| | | BE297544 R94182 AW630108 T53723 D58853 H78073 H80594 BE299560 |
| | | T48899 H70196 M17426 N77077 S77035 H58384 H61664 H78540 T84527 |
| | | C17198 H60255 H71980 R92644 W79050 X00910 M29645 R91055 M17863 |
| | | M17862 T71815 BE299561 BE464561 X06260 R94741 T54216 C18594 |
| | | BE262015 X06161 AW409889 AA378400 BE263228 BE313278 R88116 |
| | | BE313457 H43500 T48617 BE313761 H77309 AI207601 X06159 H40413 |
| | | X03425 T87663 R10627 X03562 M14118 W03982 R97520 H81229 T83157 |
| | | H83168 H48762 AA669898 BE263054 H47289 AA022807 R11555 H74260 |
| | | R76968 R28338 H72534 H72464 H62031 N72478 N45355 AW411300 |
| | | R89113 R69135 H58454 T83281 R93476 H69645 H68015 T82229 H71089 |
| | | T85121 H59939 W65299 N78176 H53909 N72373 R21788 H04660 H59639 |
| | | H61874 BE262219 T53614 N73335 N50464 W00943 N77189 R89257 AA570502 |
| | | R89432 R06366 AA553480 AA776271 AA551359 AA551050 H51670 AA601052 |
| | | BE299081 H68198 H52276 BE207832 N91192 H70332 X07868 X07868 H69464 |
| | | H53782 H73710 R80435 AA553384 AW884176 N53475 T71662 AW954036 |
| | | AW954033 AA552931 H93206 AA430218 AA553476 AI918470 T54124 |
| | | BE207982 BE300177 N73994 AW882625 N39549 N53838 AA722389 H71878 |
| | | H58909 H37849 H78435 T47933 R77174 R83814 AA411890 H94199 |
| | | AA663208 BE205778 AA490137 H70492 R98232 H37800 AA679294 H40341 |
| | | H74238 H47290 H73231 T48618 AA025428 AI039521 H92969 N59389 |
| | | H80538 H72933 T90630 AA411891 N55000 H74225 AA340290 AW957061 |
| | | T54316 AA340437 H57125 H58908 H79027 H63450 N74623 R93425 H68714 |
| | | H68758 N68396 H48763 N69256 H57320 H53831 H53589 N68833 N52453 |
| | | H56048 H69870 H78074 R69253 R83375 T53615 H94330 H58455 H90864 |
| | | T47934 H74261 R89258 R97997 R91056 R28339 R86760 H78235 R97521 |
| | | H67692 H40358 AA022688 H52513 H59601 T88690 H65256 H63397 W65397 |
| | | AA553588 R19280 N52645 W73930 R06367 R21743 H72372 N73921 |
| | | AW883539 AW882639 T40616 H47084 R95723 AA634316 AA862781 H77310 |
| | | R91389 H93111 R92767 T54512 R89341 H70333 H57817 H82941 H62032 |
| | | N52638 H58385 T91796 H51086 AA340292 T49918 H81230 R36121 |
| | | N50411 T87664 N62436 N39340 AA665637 AA340446 H93377 H92973 |
| | | BE296290 BE269788 H61665 AA340444 N54605 AA454101 R10628 R94200 |
| | | AI200549 AA342640 BE298855 BE250229 T49916 H82008 N28278 AW880662 |
| | | H71268 N76791 H47685 H65255 W05198 AW889144 N76677 H71702 H68036 |
| | | H71915 R91612 R87807 H68059 AI133328 AI247866 AA621443 AW881050 |
| | | AA700847 AA340413 AW878608 AW881181 AW878249 H71916 N54596 |
| | | BE161581 AW878082 W04212 AW881040 AW885492 AW880519 AA334887 |
| | | AW878715 W06882 AW630222 AW885381 H70869 AW381778 H47601 AW889982 |
| | | H63868 AW884986 AW878713 AW878685 R36391 AW878694 AA368070 C03393 |
| | | AW878695 AW878705 AW878665 AW878742 AW878620 AW878823 AW878688 |
| | | R29048 AW878690 AW878686 AW878810 AW878827 AW878733 AW878659 |
| | | AW878749 AW878681 AW883353 AW883277 AW883300 AW883565 AW883298 |
| | | AW883143 AW883045 AW883482 AW883352 AW883417 AW883357 AW883231 |
| | | AW883474 AW883355 AW882620 AW882533 AW883754 AW883139 AW882827 |
| | | AW883641 AW883567 AW883481 AW882983 AW882982 AW882465 AW883419 |
| | | AW882466 AW883639 AW883230 AW882981 AW882534 AW882874 AW882619 |
| | | AW883480 AW882826 AW882831 AW882835 AW882830 AW883563 AW882456 |
| | | AW627642 |
| 117156 | 145392_1 | W73853 AA928112 W77887 AW889237 AA148524 AI749182 AI754442 |
| | | AI338392 AI253102 AI079403 AI370541 AI697341 H97538 AW188021 |
| | | AI927669 W72716 AI051402 AI188071 AI335900 N21488 AW770478 W92522 |
| | | AI691028 AI913512 AI144448 W73819 AA604358 N28900 W95221 AI868132 |
| | | H98465 AA148793 |
| 131859 | 3672_1 | AW960564 AA092457 T55890 D56120 T92525 AI815987 BE182608 BE182595 |
| | | AW080238 M90657 AA347236 AW961686 AW176446 AA304671 AW583735 |
| | | T61714 AA316968 AI446615 AA343532 AA083489 AA488005 W52095 W39480 |
| | | N57402 D82638 W25540 W52847 D82729 D58990 BE619182 AA315188 |
| | | AA308636 AA112474 W76162 AA088544 H52265 AA301631 H80982 AA113786 |
| | | BE620997 AW651691 AA343799 BE613669 BE547180 BE546656 F11933 |
| | | AA376800 AW239185 AA376086 BE544387 BE619041 AA452515 AA001806 |
| | | AA190873 AA180483 AA159546 F00242 AI940609 AI940602 AI189753 |
| | | T97663 T66110 AW062896 AW062910 AW062902 AI051622 AI828930 |
| | | AA102452 AI685095 AI819390 AA557597 AA383220 AI804422 AI633575 |
| | | AW338147 AW603423 AW606800 AW750567 AW510672 AI250777 AA083510 |
| | | AW629109 AW513200 AA921353 AI677934 AI148698 AI955858 AA173825 |
| | | AA453027 AI027865 AW375542 AA454099 AA733014 AI591384 R79300 |
| | | R80023 AA843108 AA626058 AA844898 AW375550 AA889018 AI474275 |
| | | AW205937 AI052270 AW388117 AW388111 AA699452 AI242230 N47476 |
| | | H38178 AA366621 AA113196 AA130023 H39740 T61629 AI885973 AW083671 |
| | | AA179730 AA305757 AI285455 N83956 AA216013 AA336155 AW999959 |
| | | T97525 AA345349 T91762 AA771981 AI285092 AI591386 BE392486 |
| | | BE385852 AA682601 AI682884 AA345840 T85477 AA292949 AA932079 |
| | | AA098791 D82607 T48574 AW752038 C06300 |
| 125565 | 1704098_1 | R20840 R20839 |
| 133607 | 1227_6 | BE273749 BE397561 BE387189 AL037858 AL037878 AI963094 BE259216 |
| | | AA011363 AL036189 BE562325 AA251169 BE617431 N98537 AA158093 |
| | | AL047800 M34539 NM_000801 AA312140 D16971 AA158904 AA307114 |
| | | AA312803 T09203 AW629686 AL048504 BE388578 AA220957 AA158364 |
| | | BE267385 AA294971 C18055 BE241757 AA115056 AI936769 BE378435 |
| | | BE206971 AW674924 BE622060 AA604674 AA115273 AW402159 AA338608 |
| | | BE568819 M80199 X55741 AA375111 AA376016 BE612671 AA805742 |
| | | AW405588 N25850 N44580 H06031 AW403549 BE536552 AA056726 BE543239 |
| | | AA082517 AI201645 AI201642 AI192622 N40104 AA370921 BE547569 |
| | | AI969602 AA302038 AI197890 AW268354 AI014938 W45448 AI541395 |
| | | AA037272 BE538826 AL039613 BE536130 AA299355 AW805147 AW974624 |
| | | H53220 AI471471 AA399303 AA007386 W35106 BE613277 R12739 R12738 |
| | | AA304342 AA687802 BE409581 AI498844 AV662092 AW904105 AA011375 |
| | | BE315214 H99302 BE537893 N32299 AW855829 AI291320 BE078322 AI301395 |
| | | AA303362 N32719 AA358328 AA357877 AI952540 H56279 H02758 H02048 |
| | | AW805233 R82224 AA410772 AA291352 BE171109 N69935 BE169248 |
| | | AA361173 H44978 BE617887 D52560 AA084043 W03595 R67219 N36477 |
| | | N42924 R67104 H44901 H79695 W21105 AA393988 W30899 AA316096 |
| | | BE622896 W46872 AA442678 BE544893 BE540112 BE621873 AA338067 |
| | | N55052 BE398154 BE621210 AA740760 C03739 C03206 BE396692 AA482370 |
| | | AA031614 AA301575 AA304710 AA132153 AA029796 AA994960 H19567 |
| | | AA442969 H49781 H46871 AA035395 AA056185 AA149378 AA643080 |
| | | AL135479 AA292329 AA654337 AA041228 AA454888 AA025039 W58331 |
| | | AA625981 T94941 AA302448 H19900 AA218956 AA513790 AA563962 |
| | | AA398076 W44441 AA293276 W47373 AA625879 W30688 AA043029 T64284 |
| | | R79151 AA304340 AA485186 AA604939 R82470 AA421425 AW771456 |
| | | AI339329 AA304424 AA605236 AA936934 AA587673 AI209162 AI697301 |
| | | AI479995 AI679814 AI361950 AW189125 AI955888 AI986019 BE301019 |
| | | AI084792 AI310211 AW189307 AI022070 AW977204 AI146825 AW190163 |
| | | AW303281 AI828345 BE046043 AW029257 AA482268 AI246507 AI420729 |
| | | AW084932 AW439514 AI890487 AW439692 AI523896 AI186612 AI659953 |
| | | AI889773 AA687527 AW072694 AW262153 AW467371 AI613269 AI679238 |
| | | D54404 AA158103 AW105527 AW149739 AW150361 AW268387 AW117708 |
| | | AI951682 AI687440 AW674285 AA678365 AI587082 AA732095 AA019899 |
| | | W45661 AA627300 BE613304 AA765891 AA612935 AI814658 AW316916 |
| | | R66594 AA514640 AA025040 AA031472 AW732076 AA029797 AI244560 |
| | | AI128734 AW381720 AI092360 AI263283 AW613175 AI890675 AI720156 |
| | | AW631348 AI635106 AI278045 AA303979 AA703505 W45449 AW078661 |
| | | AI292052 AW381707 AI147854 AW381743 AA158905 AA303258 AA888144 |
| | | AW195967 AA428706 AA989559 AA617731 H19882 BE543418 AA830386 |
| | | AA421302 W58652 T94995 AI869743 AI679145 AW085971 N98425 AA765136 |
| | | AI347027 AI356955 AA928038 AI679717 AA458459 AA679281 AI367973 |
| | | AI270041 AA765135 AA732793 AI798447 AA668646 AA251008 AI984538 |
| | | AI401737 AA056186 BE043308 AW662375 AI302110 N50724 W96332 |
| | | BE537047 N26983 AI567172 AA765296 AW673237 N29784 AA534275 |
| | | AA084044 AW067973 AW300766 T63398 W46823 R39790 AI364185 AW298582 |
| | | AA454814 AW069878 N67751 H05982 N23140 AI362647 AI302086 AI767772 |
| | | N25755 H53114 AA706133 T93511 AA429291 AA935294 AA987647 W02803 |
| | | R66595 AI680795 W23673 AW440794 AA722872 H49538 AW131042 AA531603 |
| | | AA908665 AA040791 AA235312 W52205 N93444 R82180 H02759 H79696 |
| | | AW088894 H56079 AA961143 AW067776 AW973745 AA016311 AW071227 |
| | | AA017511 AI753994 W47374 T64155 AA296092 AI698626 AA558158 |
| | | AA296088 AW794259 H01963 AA149267 AA485076 AA975856 H44938 |
| | | AA035396 AI955555 H46289 AA486161 AI631222 AA359047 AW794253 |
| | | AI806962 AW243930 AA526145 AW878734 AA018464 AA132031 R67220 |
| | | R79152 AA296093 H54300 AI005160 BE242548 AW992803 AW878644 |
| | | AW878666 T27742 R82471 AW517604 AW472738 AI282904 R39791 AA486098 |
| | | AW467891 AW960520 AA551736 AA056621 AW945197 R66373 AA554236 |
| | | BE242202 AI904376 AI832590 H19484 R00890 AI627677 AA302287 |
| | | AI869451 AI734855 AI708073 AI832902 AA585184 AW204299 AA055565 |
| | | D12417 D11975 T63543 AW664099 R54423 BE612712 T96340 T63985 |
| | | AA598917 T40735 T64053 AA149284 AW272548 AA363445 AA042893 |
| | | AW300697 BE261973 T53501 T53500 AW878729 AW878657 AW794391 |
| | | AA069193 R01553 H44875 AA385406 AA533968 M93060 AL135600 W96331 |
| | | AA017651 AA018849 AA017692 H85337 BE278690 AA731598 AA018512 |
| | | AI076813 AI022644 R02585 X52220 AW296894 AA825671 AI699321 |
| | | AI393601 AW592611 AI146747 AA608921 AA158365 AW590007 AA354519 |
| | | D20081 R02704 AW798339 M92422 AA094903 AA007676 |
| 133681 | 13893_1 | AI352558 Z82248 X78138 NM_003405 AU077248 AA223125 S80794 D78577 |
| | | AI124697 AW403970 BE614089 BE296713 BE621334 L20422 X80536 |
| | | D54224 D54950 X57345 N29226 AA127798 AA340253 F08031 AA192540 |
| | | H67636 AA321827 AW950283 AA084159 BE538808 AW401377 AA256774 |
| | | C03366 W46595 W47608 AA305009 H69431 H69456 AL120082 H11706 |
| | | AA303717 AA361357 H22042 H78020 AW999584 AA134368 AA322911 |
| | | AA322961 H60980 N85248 N31547 H79624 T11718 W85826 AW894663 |
| | | AW894624 BE167441 BE170015 AA304626 AW602163 AW998929 AA156681 |
| | | AA151067 BE002724 AA608688 H82692 BE155392 AW383636 BE155394 |
| | | AA487004 AW383504 AI342365 R82553 W16498 BE155344 AI143938 |
| | | R69901 AA322873 AW340648 R25364 AA367935 AI559406 AA033522 AA374252 |
| | | AW835019 AI922133 AI697089 N99662 AW189078 AI199076 AW151598 |
| | | W59944 AA662875 W94022 AA299055 AI039008 AI829449 AA583503 |
| | | AI635674 AW131665 AI473820 AW273118 AW900930 AA908944 AI688035 |
| | | AW170272 AI082545 AW468176 AI608761 AI082748 AI911682 AI248943 |
| | | AI831016 AA192465 AI218477 AA938406 AA385288 AI809817 AA905196 |
| | | AI191245 AI470204 AI188296 AI421367 AI125315 AI087141 AA629032 |
| | | AA740589 AI554181 AA150830 AI248541 AI077943 AA775958 AA864930 |
| | | AI261476 AI123121 AI310394 AA862331 AA872478 BE537084 AI205606 |
| | | AA720684 AI872093 AW150042 AL120538 AA219627 AA988608 C21397 |
| | | AI359337 H25337 AI089749 AA605146 AI359620 AA150478 AI359738 |
| | | AW383642 AW995424 AI766457 R56892 AI089839 W61343 N69107 W46459 |
| | | AA565955 N20527 AI279782 W46596 AA776573 H23204 AI866231 AI083995 |
| | | N21530 AA126874 D82630 W65437 AI086917 AW382095 AI086877 H69844 |
| | | AW340217 W85827 L08439 AA262704 AA505380 W47413 W94135 AA223241 |
| | | AW089153 AA084101 BE538000 AA096126 T28031 AA491574 R84813 |
| | | AA774536 AW383522 AA155615 AW383529 AA491520 AW028427 AA171496 |
| | | AI469689 AW664539 AI811102 AI811116 BE464590 BE350791 H78021 |
| | | T15405 H21979 AA219489 H13301 AA505883 AI864305 AI423963 AW084401 |
| | | F04963 R69858 H67097 AI917740 AI655561 H69864 AA033631 AW383484 |
| | | AI886261 H25293 AA513281 AW271187 H11617 N79982 AI174338 AI904207 |
| | | AI904208 BE614558 W94127 W65436 AI272249 AA700018 AI579932 |
| | | AI085941 AW152629 |
| 134403 | 17037_1 | AA334551 BE008229 AA307537 AW961156 AW995894 AW995826 NM_006751 |
| | | M61199 AA045603 AL036372 AV645606 AI688095 AW351901 AA101337 |
| | | AA101345 N73342 BE018030 BE569044 AW841975 AA373388 BE090412 |
| | | H95440 N53845 R67867 AA093441 AA363427 H93708 AW023134 AW994986 |
| | | AW994989 BE090429 R23614 AI567932 H03726 H01101 H01867 AA548743 |
| | | AI671806 AW872949 AW872941 AA742447 AI199788 AA045604 AI637465 |
| | | AI741796 AW242217 AW131463 AI765302 AI683923 AA889762 AI804889 |
| | | AI986437 C06049 BE502340 AI695651 AI491970 AA496804 AA281008 |
| | | AA665699 AI473814 BE301445 AA707837 AA551925 AI017348 AI208185 |
| | | AA775203 AA156296 AA557463 H95441 AA768547 AW769358 AA991197 |
| | | AA181954 AI091389 AI147289 AW771837 AI638582 AA844411 AI374750 |
| | | T29320 AW951272 AW085923 H02834 AA843259 AA814696 AW183290 |
| | | AA158453 N68125 N69039 AA100423 AA101346 AI918720 H01102 R67868 |
| | | H01868 N66438 R46580 AI858433 AA599560 AA187577 AA157481 AA361520 |
| | | AL047827 AA158452 R21688 AW964874 AA325161 R40871 AW752395 |
| | | AW375924 R13355 AA281174 AA428908 |
| 126872 | 142696_1 | AW450979 AA136653 AA136656 AW419381 M984358 AA492073 BE168945 |
| | | AA809054 AW238038 BE011212 BE011359 BE011367 BE011368 BE011362 |
| | | BE011215 BE011365 BE011363 |
| 121335 | 279548_1 | AA404418 AI217248 |
| 130018 | 18986_1 | AA353093 AW957317 AW872498 AI560785 AI289110 AW135512 X97261 |
| | | T68873 |
| 121822 | 244391_1 | AI743860 N49543 AW027759 BE349467 AI656284 BE463975 R35022 |
| | | AA370031 AW955302 AL042109 N53092 AI611424 AL079362 AI969290 |
| | | AI928016 BE394912 BE504220 BE467505 AI611611 AI611407 AI611452 |
| | | W56437 AI284566 AI583349 AW183058 AI308085 AI074952 AA437315 |
| | | AA628161 AW301728 AI150224 AA400137 AA437279 AI223355 AA639462 |
| | | AI261373 AI432414 AI984994 AI539335 AA401550 AA358757 AI609976 |
| | | AA442357 AA359393 AA437046 AA370301 AA429328 AW272055 AI580502 |
| | | AI832944 AI038530 AA425107 AI014986 AI148349 AW237721 AW779756 |
| | | AW137877 AI125293 AA400404 R28554 |
| 123523 | genbank_AA608588 | AA608588 |
| 123533 | genbank_AA608751 | AA608751 |
| 125091 | genbank_T91518 | T91518 |
| 123964 | genbank_C13961 | C13961 |
| 102491 | entrez_U51010 | U51010 |
| 118475 | genbank_N66845 | N66845 |
| 118581 | genbank_N68905 | N68905 |
| 113947 | genbank_W84768 | W84768 |
| 101447 | entrez_M21305 | M21305 |
| 101667 | 13349_1 | NM_005381 M60858 AW373732 AW373724 AW373689 AW373629 AW373609 |
| | | AW373776 AA187806 AW386946 AW374207 T05235 AA216203 AW385556 |
| | | AA306940 AA306526 AA315461 AL036757 AW373711 AW403124 AW403640 |
| | | AW377084 T27360 H62638 F06957 AW377051 AA554779 AA378568 AA096007 |
| | | AW352407 AW302637 F07929 H17433 AW382712 H05665 F07292 N39875 |
| | | AA089729 H62556 N42842 R12952 AW373735 AW364155 AA056183 W39185 |
| | | AW382708 N32488 AF114096 AW375993 AI133569 W52561 AA603040 |
| | | AA133710 AI928796 AW176370 AA827519 AW338437 AA521142 T29341 |
| | | AI800461 AW317002 AA703914 AA860830 AI859203 AI445772 AA714334 |
| | | AI817066 AI832027 AW510442 AI635802 AW088306 AW068672 AW408555 |
| | | AW467542 AA552657 AA152367 W32081 AA582124 AA074040 AA931657 |
| | | AI051154 AW410203 AI921644 H17434 AI832330 AW404836 AI925038 |
| | | AA088423 AA954166 AA580453 AW021292 AI267215 AW080082 AW383778 |
| | | AI933053 AI919097 W31557 N90245 AA931591 AA563995 F36352 AA056184 |
| | | AA476294 AA641327 AA533550 AI749630 W58323 AA569119 AA508573 |
| | | AI809050 AI378996 AA411362 AW407505 AA938104 AA074041 AA632876 |
| | | AW193748 AA507873 AI270128 AI472365 AA411363 AI523216 AI719965 |
| | | AI816302 AA182681 AI707990 AA133588 AI758537 W60253 AI460308 |
| | | AA135423 AI083904 F04188 N89693 AW408776 AI678595 AI270568 |
| | | AA722059 W58234 F33650 AA090547 AA285108 AA425981 N85079 D20218 |
| | | AI273980 AA159028 F03226 AW247914 N26918 AW272741 N90109 H05666 |
| | | N23327 AW247953 R44748 AA962015 F03558 AI752394 AW409913 AW248396 |
| | | AI816463 AI752393 AA325370 AA263089 AI570130 AI971951 AI160658 |
| | | AI357360 AW168686 AL121075 AW050536 N21672 W67748 AA514242 |
| | | AI127386 H14607 AI185752 W79364 AA088520 AA152476 AW351940 |
| | | AW373683 AI940524 AW374953 T56500 N24329 AI940720 AW374933 |
| | | AW374947 AW391913 AL138337 AW376241 AW062943 F26666 AW410202 |
| | | AW062958 F34529 AW381807 AW393315 W17147 AW176359 AA664576 |
| | | AW380424 AA306040 AI745674 AW300951 AI188579 AI438973 AI305271 |
| | | AA433818 AA612807 AI831809 AI940409 AA158663 AI572988 |
| 108931 | genbank_AA147186 | AA147186 |
| 103138 | entrez_X65965 | X65965 |
| 103432 | entrez_X97748 | X97748 |
| 119174 | genbank_R71234 | R71234 |
| 133678 | 11235_1 | AW247252 AA346143 NM_000270 AA381085 N91995 X00737 AA381079 |
| | | AA296473 AA296110 AA315735 AA311617 AA326750 AA376804 AW403290 |
| | | T95231 M13953 T47963 H82039 AA279899 AA627997 N76320 N99527 H37842 |
| | | W20095 AA457308 AW469547 AA724143 H83220 AA319496 W86334 W30892 |
| | | R89169 R99427 N41854 H47286 AA348094 AA045089 R63016 AI922219 |
| | | AI024906 AI096488 AI885005 AA194872 N90489 AI452544 H72411 AA282427 |
| | | AA430735 R68963 R22453 H70385 AW129369 AW467320 AW519082 AA345018 |
| | | AA582183 AI961789 R65918 N30611 AI979189 AI280889 AW273191 R66531 |
| | | AI285845 AI675927 AI421990 AW190879 H37794 AA699667 H68427 AA954388 |
| | | AI188757 AI140048 AA430382 AI204151 AW247864 AA559099 AI431420 |
| | | AA548276 AI149466 AA772669 AA694388 AA724168 AA301651 AA281952 |
| | | AA779925 AA234760 W86290 AA913603 AW511745 AI500697 AA814922 |
| | | AA835040 T47964 H53998 AA975804 R98710 AI077604 N70252 R98084 |
| | | AW250171 H69268 AI597614 AA970746 AA972548 AI377116 R62962 H16737 |
| | | R89070 AA731329 R66532 N54354 AI818832 H81944 N71567 T95122 |
| | | W86463 AA437095 AI431999 AI915724 N63851 AI674743 AA457307 |
| | | AA211475 N64444 AI799146 H72853 R99335 H60413 AA770367 AA156105 |
| | | AI269937 H64029 H89728 R65819 AW470496 AI873318 AI735713 H82987 |
| | | C02447 AI478666 T27651 AI699770 AW025156 H69719 AI984717 N69225 |
| | | AI459856 AA953577 AI424691 H13843 R22404 AI873796 AI336002 |
| | | N70898 AI420854 AA541792 AA346142 AI000814 AI828348 AA045090 |
| | | T51257 N90434 H13890 N73184 AI708083 AA781606 AA329050 AA339985 |
| | | R68964 H64795 W04186 H16845 |
| 119416 | genbank_T97186 | T97186 |
| 119559 | NOT_FOUND_{—} | W38197 |
| | entrez_W38197 | |
| 123473 | genbank_AA599143 | AA599143 |
| Table 4A shows the accession numbers for those pkeys lacking unigeneID's for Table 4. The pkeys in Table 7 lacking unigeneID's are represented within Tables 1-6A. For each probeset we have listed the gene cluster number from which the oligonucleotides were designed. Gene clusters were compiled using sequences derived from Genbank ESTs and mRNAs. These sequences were clustered based on sequence similarity using Clustering and Alignment Tools (DoubleTwist, Oakland California). | | |
| # The Genbank accession numbers for sequences comprising each cluster are listed in the “Accession” column. | | |

Pkey: Unique Eos probeset identifier number
CAT number: Gene cluster number
Accession: Accession number used for previous patent filings

TABLE 5

| | |
|---|---|
| Pkey: | Unique Eos probeset identifier number |
| Accession: | Accession number used for previous patent filings |
| ExAccn: | Exemplar Accession number, Genbank accession number |
| UnigeneID: | Unigene number |
| Unigene Title: | Unigene gene title |

| Pkey | Accession | ExAccn | UniGene | UnigeneTitle |
|---|---|---|---|---|
| 115819 | AA426573 | AA486620 | Hs.41135 | AA486620 |
| 132837 | D58024 | AA370362 | Hs.57958 | AA370362 |
| 101545 | M31210 | BE246154 | Hs.154210 | BE246154 |
| 102898 | X06256 | NM_002205 | Hs.149609 | NM_002205 |
| 101192 | L20859 | BE247295 | Hs 78452 | BE247295 |
| 102915 | X07820 | X07820 | Hs.2258 | X07820 |
| 105330 | AA234743 | AW338625 | Hs.22120 | AW338625 |
| 107385 | U97519 | NM_005397 | Hs.16426 | NM_005397 |
| 102024 | U03877 | AA301867 | Hs 76224 | AA301867 |
| 134416 | M28882 | X68264 | Hs.211579 | X68264 |
| 103036 | X54925 | M13509 | Hs.83169 | M13509 |
| 104865 | AA045136 | T79340 | Hs 22575 | T79340 |
| 106124 | AA423987 | H93366 | Hs.7567 | H93366 |
| 105330 | AA234743 | AW338625 | Hs 22120 | AW338625 |
| 109001 | AA156125 | AI056548 | Hs.72116 | AI056548 |
| 104764 | AA025351 | AI039243 | Hs.278585 | AI039243 |
| 133200 | AA432248 | AB037715 | Hs.183639 | AB037715 |
| 105263 | AA227926 | AW388633 | Hs.6682 | AW388633 |
| 105178 | AA187490 | AA313825 | Hs.21941 | AA313825 |
| 109456 | AA232645 | AW956580 | Hs.42699 | AW956580 |

TABLE 5A

| Pkey | CAT Number | Accession |
|---|---|---|
| 115819 | 10241_1 | AA486620 AF205940 AA297524 AB034695 AA081335 NM_016242 AA188323 |
| | | AA297537 H88204 AW953081 W31695 AW582203 AA248250 AW681211 |
| | | AA426230 AA464807 AA426155 N44141 AA347390 AA770661 AI333225 |
| | | N36136 AW665724 AA431894 AI374976 AI400254 AI338446 AA186695 |
| | | H88205 W04527 AA487066 AI051414 AA918383 AA426573 AA425620 |
| | | AW438654 AA090513 BE167284 BE167291 AI301726 |
| 102024 | 14505_1 | AA301867 AW957981 R27614 AA155808 AI920990 AI740711 AA301026 |
| | | AA301015 AI220981 AI857670 AI537140 AW015210 AA030000 W46890 |
| | | H44021 AI355967 AI651735 AA058479 AA146932 T58265 R85890 AA047810 |
| | | AA017387 AW026093 AA971133 AI827263 AI056416 AI355994 AI127691 |
| | | H46603 U03877 NM_004105 AA157357 H42844 AA146824 AA187709 |
| | | AA187269 AA304348 AA147292 AA361687 AA156041 AA330636 R32929 |
| | | AA321130 AW950260 AA082157 AA029129 AA303708 AA028155 D31561 |
| | | T84689 AA302493 BE153057 BE153181 W39408 AA187200 BE153250 |
| | | AW383337 AW382622 AW382647 AW750072 BE153060 AW382630 AW371865 |
| | | AW392464 AW382664 AW382658 AW382650 H61647 AW365075 AW365049 |
| | | AA373397 BE072779 BE072781 Z30254 W24381 BE153254 AA040442 |
| | | BE072729 BE072731 N94740 AA146945 AW802737 AI826799 AI085395 |
| | | R34034 H65140 AA082800 H88275 AA147824 R63882 W80899 AA296413 |
| | | AI765300 AI862426 AW022055 AW300003 AI743784 AI862635 AI985428 |
| | | AA147764 AW573245 AW190290 AI040898 D57613 N63457 AA148082 |
| | | AI028458 AA148110 AW814489 N75105 AW629443 AA704122 AW582220 |
| | | AA181240 AA057495 AI418224 AI261751 AW388595 AI472205 AW470672 |
| | | AA102546 AA789046 AA182416 AA062668 AW300732 AI288220 AA181982 |
| | | AA146825 AA028130 AI985522 AA303344 AA081313 N69082 AA182035 |
| | | AI867128 AA100902 AA605087 N67178 AW020324 AW890446 AI472191 |
| | | AI335691 AI597837 AI081143 AI335681 AA040443 AI128067 AI678244 |
| | | AA018303 AA157260 W80792 AI934590 AI096430 T54343 AI446350 |
| | | AA165196 AA780683 AA603631 AA047787 AA968580 AA912645 AW890504 |
| | | AW026913 D56983 H52088 AA156121 R30848 AW023036 AI590960 N67345 |
| | | AI753225 AI753283 AI183768 AA147818 H89101 AI362141 H89205 AI147711 |
| | | AA321129 AA668622 AA343479 AW069438 AI422376 AW629270 AA013413 |
| | | AI221948 AA970605 N52335 H38366 T91180 AA657841 AA017386 AA152227 |
| | | AA187593 AI913340 AI719313 AI969943 AI701271 AI004328 AI868348 |
| | | N93659 H65093 H25736 D57007 D56957 C00987 D61839 D56661 AI472137 |
| | | AI971002 D56971 BE048830 D57972 AI589286 AI361055 AI361071 |
| | | AI292223 AA155898 D57139 D57981 D57345 AI420034 D57332 D57959 |
| | | AA875933 R33493 N67558 D58353 AA188394 AA147966 AI160640 AI363165 |
| | | H40638 AA578137 AW950265 AA300943 AI128999 H46584 AA917355 N57820 |
| | | AA320504 H51959 H25737 |
| 101545 | 24607_1 | BE246154 M31210 NM_001400 AA193392 NM_016537 AF233365 AF022137 |
| | | H27787 AA370448 F05373 T27666 W21494 AA036907 AI249966 N93476 |
| | | F01623 AA304390 AA308808 |
| 109456 | 180633_1 | AW956580 AA886361 AI147670 AI090115 AI168683 AA232645 H99504 |
| | | AA374707 AA380875 AW139567 AI735132 BE439385 AW629780 N28322 |
| | | AA232789 AA232790 N73285 |
| 103036 | 17145_1 | M13509 X54925 NM_002421 M16567 X05231 M15996 W39354 AA186634 |
| | | AA852324 AA187507 AA081149 AA186524 AA187264 AA187361 AA386155 |
| | | AA186973 AA374217 U78045 AA081230 AA188049 AA186393 W56827 AA852602 |
| | | AA157468 AA308204 AA186754 AA186808 AA082516 AA304334 AW376428 |
| | | BE439384 AW376420 AA156273 T18504 AA186521 W49496 AW084608 |
| | | AA083575 AA372360 AW963590 AA132297 W47445 AA186376 AA157628 |
| | | AW003999 AI037890 AI858060 AI589010 AI743739 AI452673 AW304188 |
| | | AW117854 BE439933 AA157416 AW778966 AI038497 AA081006 AA100829 |
| | | AA181048 C02231 T27821 W23960 AW954802 AI471432 AW801296 AW801289 |
| | | AW801603 AW801523 AW801292 AW801542 AW801601 AA181134 AI445147 |
| | | AA191501 AA582862 N94407 AI147810 AA181880 W49497 W52714 AA188249 |
| | | AI932881 AI082493 AA503656 AA182682 AW801393 AA182830 AA181882 |
| | | AA182826 AI613182 N94510 W47343 AI085755 AI076956 AI918426 |
| | | AA081208 AI282835 AA147528 AI081490 AI654536 AA181875 AA081282 |
| | | AA186389 C06085 AA083542 AI800644 AA157642 AA101069 AA157752 |
| | | AA158121 AA143331 AA081283 AA852603 AA188296 AI932880 AW449628 |
| | | AA187348 C02091 AA514656 AA082736 AA308786 AA143201 M16567 |
| 133200 | 28960_1 | AB037715 AI351347 AI375796 AI884765 AL121124 W01068 AI807275 |
| | | T95240 R42807 AW515645 AI057314 AI033520 AA057671 N70215 AA054215 |
| | | AW204183 AA552149 T95130 AW796310 AI866520 AW275564 AW796308 |
| | | AI637901 AW197404 T78406 AA456232 AW206463 AA779800 AI052696 |
| | | AA026744 AA454623 AW470729 R45490 AW770258 AI038393 AI290170 |
| | | AA722734 AL121125 R41608 AI862414 AA838611 R45582 AI278083 |
| | | BE466849 BE219944 AA418030 BE041555 AA578572 T16528 AW006344 |
| | | Z39782 AI244848 AW137344 AA707400 AI032028 BE540464 AI094265 |
| | | AI184281 AA931890 AW382744 AW382729 AW020448 AW827237 AA431226 |
| | | AI672059 AW772345 N70172 AW022003 AI862704 H19344 R61511 AI080204 |
| | | H16566 AA432248 AI767980 T16688 AI984342 AI217478 AI767095 Z38551 |
| | | AI359566 AI361437 AI041000 R07033 H16608 H19054 R12874 R61567 |
| | | N98368 BE221199 Z42320 AA094554 R07078 AW860886 AA418090 R41262 |
| 132837 | 256666_1 | AA370362 AA364110 AW959554 AW371737 AW382068 AW604716 AW604713 |
| | | AA487827 AW371674 AA429137 BE503321 T93570 W72803 AI093076 |
| | | AA487977 AI241562 BE439445 AW204065 R51635 AI802994 T10362 W68553 |
| | | AI866215 AW152154 AA700716 AI127443 R15824 AI537587 AA953110 |
| | | D58024 AI520811 AA693670 AI453280 W76329 AW023955 AW022563 |
| 102898 | 24023_1 | NM_002205 X06256 M13918 BE070866 AW239485 AW996127 BE273894 |
| | | BE272590 BE410252 R25975 T11786 T11787 AA301142 AA301165 AW960506 |
| | | BE272819 AA386086 T39391 AA285303 AA370580 D58585 T58668 AA156213 |
| | | W24142 AA343323 AW796067 AA151197 AA376121 R94782 AA302363 H90357 |
| | | R82621 AA301677 H55997 AW796059 W92358 AL046458 AA471198 AA301952 |
| | | R46287 R82694 H03186 AA187706 R32562 R27094 R25947 R25320 AW949809 |
| | | H13505 H79049 R32403 H11213 R39710 H49765 H21142 H21006 AA417664 |
| | | W52075 N56771 AA284240 N98556 N30907 AA707335 AW603781 AI340367 |
| | | AI814584 AA524182 AA370076 AA418785 AA704082 AI806851 H25513 T56388 |
| | | AA419627 H03986 H20963 T56245 AI459715 AW973768 AI334096 AI693020 |
| | | T63414 R82646 AW167251 H55998 AI274916 AA778367 AI755253 AI033667 |
| | | AW083222 AA181979 R26865 AA661627 AA706329 AI798648 AA612799 |
| | | AI160180 AI274973 AI039264 AA301880 AI042429 AA307632 AI085688 |
| | | AI278366 AI498890 AA303865 AI954844 AA502380 AA156334 AA723480 |
| | | AI803584 AI581026 AA304584 N51038 R94702 R69814 AW150962 AI570049 |
| | | AA588807 AA151198 T53400 AI567709 AI185326 AA309205 AW338969 |
| | | R53903 AA991891 AA301643 AI493337 AI026049 H25514 AI741075 |
| | | R28632 AW166445 AI333068 H49978 H91267 AA558193 AW079663 AA627380 |
| | | AA807401 AI199956 AA666118 AI718216 AW193228 AI077745 AI500496 |
| | | AI266059 AW080383 R06468 R26757 R32404 AA716599 W92322 AI077734 |
| | | AI270181 R46198 AI217540 AA304045 AA305421 AW074445 AI468256 |
| | | AW089568 AW571605 BE162930 H41009 AA578313 AW874497 AA181284 |
| | | AA861947 T29451 D20841 T58618 AA418731 AI282500 AW081407 AA604560 |
| | | AA729855 AI262538 AI580225 |
| 102915 | 2903_2 | X07820 NM_002425 BE271570 AI263526 AW296143 AI829878 AI973162 |
| | | AI085155 AA857496 AA709305 C02220 |
| 134416 | 30694_1 | X68264 NM_006500 AF089868 BE257461 BE275425 AW997154 AI902799 |
| | | AI902803 M78206 AA085691 AW392972 AA325490 BE006161 AA349269 |
| | | AA323568 AL042548 AA191148 AA187703 AA322791 AJ297452 T11625 |
| | | AW366487 AA303513 AA186961 AA173480 N28330 N28379 W40320 AA187118 |
| | | H03695 AA402709 BE407476 H06354 BE276589 AA351284 AA379921 |
| | | AL138060 BE410587 AA113094 AA340481 BE277483 R21191 R79518 |
| | | N86170 AA320505 AA296065 AW951900 AA658897 AA650052 AA654304 |
| | | AA191691 N26649 AW080963 AI265800 N72019 AI453458 AA092563 |
| | | AA402310 AI439450 AI061054 AA302358 T71566 AA302047 AA303432 |
| | | N21289 H27357 AA303504 AI174583 AW151762 AA181958 AW880618 |
| | | AA630773 AI889539 AW901058 AI373405 AA341941 AA086217 AI675590 |
| | | AI653936 AA633570 AA987619 AI270656 N93847 N40689 AW517517 N20030 |
| | | W95985 AA303955 H89170 AA309917 N21642 AA373132 W38517 AI687806 |
| | | W76182 AA101065 AA036916 N45635 AI744510 AI669803 AI039157 |
| | | AI126355 AA634607 AW131120 AW196838 AA190601 AA911130 BE221320 |
| | | N92355 AA036752 H03696 AA588873 AI458868 AI041818 AA090477 |
| | | AI093248 AA304755 AL137942 AL044688 AI083709 AI150965 N88891 |
| | | AA635675 AA594898 W94657 AA182823 AW166205 F27886 R79246 F37329 |
| | | AA565697 AI075739 AI088654 AI094287 AI204256 AA095203 T93020 |
| | | AA688298 AA057324 N23442 AA075411 AA305046 AI031688 AI191503 |
| | | AA111887 AA112264 N27929 AA187509 AI375522 AI474006 H06297 |
| | | AI826177 N48880 H28333 AA075490 R22809 W79542 AI055934 AA042901 |
| | | AA173481 AA301986 W74531 AI051747 AA187715 AI888888 AA993017 |
| | | AI057530 T92954 N80227 AW273595 AI351260 AW170643 AW292979 |
| | | AA302605 AA302330 BE349495 AA328602 AA302361 AI470984 AA155943 |
| | | AA155914 |
| 105178 | 7792_1 | AA313825 AW960347 AF223468 NM_016613 AA186345 AA186508 AA081195 |
| | | AA147972 AA346943 AW961667 AA187222 AA187207 AW371052 AW449751 |
| | | AW748803 AW391606 AW371047 AW371057 AW371085 AW362895 AW371092 |
| | | AW377556 BE010930 AI016882 AA247878 C04398 C05158 F11398 AA188315 |
| | | H23385 R55086 H15346 AA029106 AA228114 H17005 F08498 Z43376 |
| | | AA095582 AA055186 AA463361 R15218 AA299132 AW103578 W21538 |
| | | AA428131 AA187115 AA157197 AA157167 AW371371 AA363562 AW965995 |
| | | N55663 Z17878 AA228023 AI140342 AA100927 AA496988 AA055917 |
| | | AI089303 AW014967 AW090248 AW338371 AW131066 D62963 D79713 |
| | | AI583950 AI336781 AI500705 AI471485 AW090239 D79784 D61847 |
| | | D62789 D61842 AI086327 AI273381 D61815 D63043 AI913548 AI280560 |
| | | AI510828 AA029996 C16343 C16513 AI075741 AW516308 AI804764 |
| | | AA948068 AI356588 AW103452 AW573063 Z39445 C16489 AI949870 |
| | | F04712 AA147823 AW026284 AI151538 AA081303 AA613890 AI251865 |
| | | AW086499 AA992111 AI862091 AI373465 BE502094 AI922270 AA884288 |
| | | AA157079 N56963 AW189145 AA428080 R55056 AA884068 AW771716 |
| | | AA186662 C16364 H15723 AI921181 AA156888 H17006 AA187490 AI400994 |
| | | AA346942 H28533 AW129047 R41656 H14636 AA995041 D58370 Z21131 |
| | | D58186 AI383271 AA643977 D58044 AI934302 AW779425 F09065 H14930 |
| | | AA890693 H23274 |
| 105263 | 178672_2 | AW388633 AW378440 AW388283 AW388339 AW388333 AW388414 AW388413 |
| | | AW388607 AW388453 AW388687 AW388480 AW388591 AW388711 AW388511 |
| | | AW388438 AW388570 AW388449 AI694383 AW237145 AI652991 AI964041 |
| | | AW366319 AW366321 AW961938 AW469211 AI634155 AI492186 AI624430 |
| | | AI677965 N26502 AI963871 AW378431 AW378421 AI015391 AW352126 |
| | | N59336 AI352317 AW197113 N67998 AW778935 AI476054 AI206626 |
| | | R37116 R40211 AA227926 AA639698 R38073 AI001745 T32854 AI619649 |
| | | AI423703 F10774 AW388615 T16595 H05894 |
| 105330 | 182497_1 | AW338625 R43226 R51640 AI307645 AI308100 AI085787 AI420357 |
| | | AI692610 AA877160 AI953366 AA234743 |
| 104764 | 90967_1 | AI039243 R68234 AA025351 AA971063 AI537757 AA025362 R81636 T86650 |
| 104865 | 102037_1 | T79340 AI742317 AW182676 AW451460 AI420964 R43284 AA088179 |
| | | AW590886 AW269529 AA045187 AI521736 AI827455 AA045136 AW271709 |
| | | AI004344 AA639631 AA744417 AA744218 AA045357 AA045351 |
| 106124 | 54542_1 | H93366 AI653547 AA336265 AW966175 BE566451 R71178 AI630656 |
| | | AA234331 N55039 AA305632 AW960431 R34044 R32254 AW020970 AW451281 |
| | | AW275041 AI636933 AI655640 AA423986 AA642466 AI684063 AI633876 |
| | | AI624897 AA814795 AW590328 AI889166 AW243541 AI439691 AW473445 |
| | | AI475516 AA741228 AI127534 AA165143 AI074714 AI654076 AA400674 |
| | | AI560249 N50709 AW438621 AI806810 AI434579 AI308184 AA423987 |
| | | AI141272 AI565586 AI338440 AA219628 AI246643 AI985809 AA724260 |
| | | AA633988 AI364172 AI798439 AI650801 R33503 AI435891 AA903649 |
| | | T96161 AA665538 AA219620 AI309962 AA400707 BE247066 R32178 |
| | | AI275962 AA661602 AW003197 BE466649 AA831198 AI620052 AI825387 |
| | | AI634037 AI670978 AI670979 AI655092 R32304 AA828858 AI382428 |
| | | AW023660 AA262892 T26891 AW089917 T26926 R32227 |
| 107385 | 6976_1 | NM_005397 U97519 AW899329 AI902387 AA077792 AA078525 AW376607 |
| | | AA077946 AA070415 BE208721 AW167958 BE293050 BE208240 AI648698 |
| | | AA101314 BE393348 BE305122 AA077591 BE274036 AA313687 BE392220 |
| | | BE378954 AA171461 AA464821 AW938242 AW938224 AW938243 AW938232 |
| | | AA147953 N64294 AA205218 AW305065 AW517478 AA307983 AA377023 |
| | | BE563629 R99976 N80294 T87719 T87928 AA496849 AA486344 AA204938 |
| | | AW370448 AA318242 AW964384 H92423 W95317 BE378774 BE391156 |
| | | AA349138 AA173095 AW513198 AA037672 AA148029 AA169726 W04791 |
| | | AA075508 BE382937 BE395034 AF139793 AA961734 N48612 H64714 |
| | | AW151251 AI565113 AI566881 AW087370 AA631168 AA622014 AW513098 |
| | | AI857810 AW152287 AI052596 AI983246 AA024856 AI912456 AI677938 |
| | | AW026403 AA972537 AI088497 AW999869 W94582 AI140166 AI160659 |
| | | AI566868 AA101263 AW190390 AW166466 AI401207 AI418156 AI625265 |
| | | AI146298 AW008592 BE223020 N58926 AI308797 AA037673 AI935992 |
| | | AI304706 AA024939 AI216589 AI610423 AI354621 AI500677 AI679389 |
| | | AI799310 N64508 AI128756 AI679897 AW589535 AA989333 AI500527 |
| | | AA565479 AA913529 AI923295 F21691 AA989376 AI699064 AA902447 |
| | | AI690910 AA772659 AA204983 AI337895 R99975 H65205 AA340766 |
| | | AI339441 AI913855 AA450293 AW192010 AA070416 N72401 AI371481 |
| | | AI247108 AI371261 AI364987 AI280171 AI269104 AI868756 AA909836 |
| | | AA983640 AI973271 AA913092 AI868205 AI144112 AI190975 N58085 |
| | | AI566638 N93405 AW150504 AW296846 AI687036 AA902984 AI824460 |
| | | AI625047 AA653148 AI611228 AW131922 AA862687 AA902519 C01732 |
| | | AW796045 AL044660 |
| 101192 | 15367_1 | BE247295 AW068092 AL041313 AA159244 NM_005415 L20859 AL135570 |
| | | W47073 AW516906 BE388271 BE408629 W46972 BE293646 BE256647 |
| | | AI075010 AL041095 AA285300 AL039560 AA368740 W26602 AA399344 |
| | | AA039235 W27631 AW834898 AW834914 R93390 AA378039 AV649660 T53674 |
| | | N98824 AA399974 AW843378 AA368267 R08256 AV653575 R27900 N48215 |
| | | AW366371 N45500 AV652967 AI889251 AI080457 N39021 AI738542 |
| | | AW242849 AI857471 AI859775 AI582830 R75850 N66564 AW341636 |
| | | AI499006 AI887217 AW026694 AW182840 AA039313 AA831346 AI393465 |
| | | AW069210 AI743830 AA744243 AA401310 AW439758 AW088152 R93391 |
| | | AA291379 AA225220 AW009358 AI192879 AA291202 AI565089 AA225089 |
| | | AA807688 AI052058 AI341641 AI066625 AA333864 AA159147 AI923912 |
| | | R75851 AI761143 AW768588 AA394195 AI288450 AW512564 AI452775 |
| | | AI056520 AA468602 AA872566 AI434739 AA291838 AI948623 AW768614 |
| | | AI374753 AW068174 AA884908 AI199346 AI199347 W94946 AI159995 |
| | | AA877642 AI280646 AI307610 AA403310 R08205 AW182123 AI000999 |
| | | R27808 AW026571 D20816 AI560350 T27667 AW960271 AI174628 AI432042 |
| | | AI424528 AA909562 T17342 AI783866 |
| 109001 | 146370_3 | AI056548 AW409843 AW263540 AA723669 AA909334 AA156120 AA157141 |
| | | AA156125 AW409866 W19499 AA157229 AW887435 |
| Table 5A shows the accession numbers for those pkeys lacking unigeneID's for Table 5. The pkeys in Table 7 lacking unigeneID's are represented within Tables 1-6A. For each probeset we have listed the gene cluster number from which the oligonucleotides were designed. Gene clusters were compiled using sequences derived from Genbank ESTs and mRNAs. These sequences were clustered based on sequence similarity using Clustering and Alignment Tools (DoubleTwist, Oakland California). | | |
| # The Genbank accession numbers for sequences comprising each cluster are listed in the “Accession” column. | | |

Pkey: Unique Eos probeset identifier number
CAT number: Gene cluster number
Accession: Genbank accession numbers

TABLE 6

| | |
|---|---|
| Pkey: | Unique Eos probeset identifier number |
| ExAccn. | Exemplar Accession number, Genbank accession number |
| UnigeneID. | Unigene number |
| Unigene Title. | Unigene gene title |
| AUC1: | 70^{th} percentile of average intensity (Al) for probeset at |
| | each of 2,6,15,24,48, and 96 hour timepoints minus 70^{th} |
| | percentile Al at 0 hrs, summed over 5 experiments. |
| AUC2. | AUC1/90^{th} percentile of Al for aorta, aortic valve, vein, and artery. |

| Pkey | Ex. Accn | UnigeneID | UnigeneTitle | AUC1 | AUC2 |
|---|---|---|---|---|---|
| 314941 | AA515902 | Hs.130650 | ESTs | 1038 | 9 |
| 327414 | | | predicted exon | 303.2 | 30.3 |
| 321911 | AF026944 | Hs.293797 | ESTs | 429.2 | 429 |
| 331578 | A1246482 | Hs.249989 | ESTs | 677.4 | 10.3 |
| 332466 | AB018259 | Hs.118140 | KIAA0716 gene product | 395.2 | 39.5 |
| 313513 | AW298600 | Hs.141840 | ESTs, Weakly similar to S59501 interfero | 324 | 32.4 |
| 320635 | N50617 | Hs.80506 | small nuclear ribonucleoprotein polypept | 394.8 | 39.5 |
| 326230 | | | predicted exon | 357.2 | 35.7 |
| 313556 | AA628517 | Hs.118502 | | 433.6 | 12 |
| 313665 | AW751201 | Hs.120932 | ESTs | −83 | 0.5 |
| 324852 | AI380792 | Hs 135104 | ESTs | 348.2 | 34.8 |
| 314372 | AL040178 | Hs.142003 | ESTs, Weakly similar to The KIAA0149 gen | −49.2 | 0.5 |
| 311877 | AA084248 | Hs.85339 | G protein-coupled receptor 39 | −1309 | 0.2 |
| 322262 | AA632012 | Hs.188746 | ESTs | −2478 | 1 |
| 312173 | A1821409 | Hs.304471 | ESTs, Highly similar to AF116865 1 hedge | −1025.8 | 1 |
| 319795 | AB037821 | Hs 146858 | protocadherin 10 | 203.6 | 5.2 |
| 313350 | AW591949 | Hs 57958 | ETL protein | 183.8 | 18.4 |
| 326759 | | | predicted exon | 1654.4 | 1.2 |
| 300318 | AW444502 | Hs.256982 | ESTs, Highly similar to AF116865 1 hedge | −346 | 1 |
| 313978 | AI870175 | Hs.13957 | ESTs | 576.6 | 2.3 |
| 306840 | A1077477 | Hs 307912 | EST | 56.4 | 0.4 |
| 310272 | AF216389 | Hs.148932 | semaphorin Rs, short form | −127.6 | 0 |
| 315044 | BE547674 | Hs 204169 | ESTs | −102.6 | 0 |
| 321325 | AB033100 | Hs 300646 | KIAA protein (similar to mouse paladin) | 1080.6 | 4.8 |
| 303251 | AF240635 | Hs.115897 | protocadherin 12 | 1270.8 | 5.3 |
| 302378 | AL109712 | Hs.296506 | Homo sapiens mRNA full length insert cDN | 915.8 | 15.8 |
| 315060 | AA551104 | Hs 189048 | ESTs, Moderately similarto ALUC_HUMAN ! | 1236.8 | 4.9 |
| 332048 | AW337575 | Hs 201591 | ESTs | 522.6 | 4.7 |
| 337214 | | | predicted exon | 269 | 26.9 |
| 311598 | AW023595 | Hs.232048 | ESTs | 796.4 | 20.2 |
| 304782 | AA582081 | | gb.nn32h08.s1 NCI_CGAP_Gas1 Homo sapiens | 316.4 | 10.5 |
| 312802 | AA644669 | Hs 193042 | ESTs | 349.6 | 7.6 |
| 302680 | AW192334 | Hs 38218 | ESTs | 638.6 | 63.9 |
| 317452 | AA972965 | Hs.135568 | ESTs | 360.8 | 361 |
| 318558 | AW402677 | Hs.146381 | RNA binding motif protein, X chromosome | 700.2 | 6.6 |
| 312149 | T90309 | Hs 269651 | ESTs | 274.2 | 7.5 |
| 319267 | F11802 | Hs.6818 | ESTs | 238.2 | 23.8 |
| 321510 | H75391 | Hs.255748 | ESTs | 231.8 | 23.2 |
| 326198 | | | predicted exon | 581.6 | 8.2 |
| 315730 | H25899 | Hs.201591 | ESTs | 281.6 | 9.7 |
| 310442 | AW072215 | Hs 208470 | ESTs | −213 | 0.3 |
| 331237 | W87874 | Hs.25277 | hypothetical protein FLJ21065 | 285 | 0.5 |
| 300469 | BE301708 | Hs.233955 | hypothetical protein FLJ20401 | 26.6 | 0.3 |
| 338316 | | | predicted exon | 1494.2 | 34.7 |
| 330968 | R44557 | Hs.23748 | ESTs | 975.8 | 1.8 |
| 331019 | NM_006033 | Hs.65370 | lipase, endothelial | 201.2 | 0.9 |
| 331261 | BE539976 | Hs.103305 | Homo sapiens mRNA; cDNA DKFZp434B0425 (f | 478.6 | 1.3 |
| 301822 | X17033 | Hs.271986 | integrin, alpha 2 (CD49B, alpha 2 subuni | 356.2 | 1.7 |
| 325544 | | | predicted exon | 1014.6 | 9.4 |
| 328700 | | | predicted exon | 627.4 | 62.7 |
| 322882 | AW248508 | Hs 279727 | Homo sapiens cDNA FLJ14035 fis, clone HE | 84.8 | 5.7 |
| 336034 | | | predicted exon | 782.6 | 78.3 |
| 316580 | AA938198 | Hs.146123 | hypothetical protein FLJ12972 | 746.4 | 13.8 |
| 309931 | AW341683 | | gb:hd13d01.x1 Soares_NFL_T_GBC_S1 Homo s | 134.8 | 13.5 |
| 330692 | R39288 | Hs 6702 | ESTs | 137 | 13.7 |
| 319962 | H06350 | Hs.135056 | Human DNA sequence from clone RP5-850E9 | 14.6 | 0.5 |
| 338033 | | | predicted exon | 540.6 | 14 |
| 314943 | Y00272 | Hs.184572 | cell division cycle 2, G1 to S and G2 to | −494.8 | 1 |
| 332640 | BE568452 | Hs 5101 | protein regulator of cytokinesis 1 | −600 | 1 |
| 338158 | | | predicted exon | 311.2 | 31.1 |
| 327036 | | | predicted exon | 351.8 | 35.2 |
| 302655 | AJ227892 | Hs.146274 | ESTs | 180.2 | 18 |
| 327568 | | | predicted exon | 229 | 22.9 |
| 324801 | AW770553 | Hs.14553 | sterol O-acyltransferase (acyl-Coenzyme | 161.2 | 16.1 |
| 317850 | AI681545 | Hs.152982 | hypothetical protein FLJ13117 | −690 | 1 |
| 322818 | AW043782 | Hs.293616 | ESTs | 126.4 | 4.5 |
| 324626 | AI685464 | Hs 292638 | ESTs | 170.2 | 17 |
| 317224 | X73608 | Hs.93029 | sparc/osteonectin, cwcv and kazal-like d | −80 | 0 |
| 310955 | AI476732 | Hs 263912 | ESTs | 466.8 | 46.7 |
| 315240 | R38772 | Hs.172619 | KIAA1106 protein | 277 | 27.7 |
| 338388 | | | predicted exon | 267.6 | 268 |
| 338442 | | | predicted exon | 256 | 25.6 |
| 318617 | AW247252 | Hs.75514 | nucleoside phosphorylase | 1247.8 | 24.2 |
| 338645 | | | predicted exon | 206 | 20.6 |
| 313135 | N58907 | Hs.162430 | ESTs | 204.8 | 20.5 |
| 324716 | BE169746 | Hs.12504 | hypothetical protein DKFZp761D081 | 203.6 | 20.4 |
| 330305 | | | predicted exon | 199.8 | 20 |
| 308248 | AI560919 | | gb.tq41g10.x1 NCI_CGAP_Ut1 Homo sapiens | 199.4 | 19.9 |
| 308886 | AI833240 | | gb.at76d10.x1 Barstead colon HPLRB7 Homo | 198.2 | 19.8 |
| 315622 | AI796144 | Hs 258188 | Homo sapiens cDNA FLJ11674 fis, clone HE | 191.2 | 19.1 |
| 323675 | R43240 | Hs.272168 | tumor differentially expressed 1 | 189.2 | 18.9 |
| 312164 | T91980 | Hs.221074 | ESTs | 187.6 | 18.8 |
| 300378 | Z45270 | Hs 235873 | hypothetical protein FLJ22672 | 271.6 | 18.7 |
| 317478 | AI343569 | Hs.107000 | Homo sapiens mRNA for WDC146, complete c | 187 | 18.7 |
| 317559 | AW452344 | Hs.129977 | ESTs | 184.2 | 18.4 |
| 317207 | AI873346 | Hs.214505 | ESTs | 182.8 | 18.3 |
| 334834 | | | predicted exon | 178.8 | 17.9 |
| 320925 | D62892 | | gb:HUM337C076 Clontech human aorta polyA | 177.2 | 17.7 |
| 303289 | AL121460 | Hs.272673 | hypothetical protein FLJ20508 | 316.4 | 17.6 |
| 328548 | | | predicted exon | 174.6 | 17.5 |
| 317108 | AA884000 | Hs.8173 | hypothetical protein FLJ10803 | 172.4 | 17.2 |
| 318013 | AI188183 | Hs 144078 | ESTs | 326 | 17.2 |
| 314299 | AW382682 | Hs 154840 | ESTs | 170.8 | 17.1 |
| 317702 | AW173339 | Hs.135665 | ESTs | 169.8 | 17 |
| 316094 | AW975920 | Hs.283361 | ESTs | 169.4 | 16.9 |
| 323706 | AA377578 | Hs.65234 | hypothetical protein FLJ20596 | 169.2 | 16.9 |
| 325843 | | | predicted exon | 321.4 | 16.9 |
| 316012 | AA764950 | Hs.119898 | ESTs | 1047.2 | 16.9 |
| 309687 | AW236154 | Hs.77385 | myosin,lightpolypeptide6,alkali,smoothmu | 168.2 | 16.8 |
| 323329 | AL134744 | Hs.10852 | ESTs | 168 | 16.8 |
| 312853 | W05086 | Hs.114256 | ESTs | 167.4 | 16.7 |
| 313070 | AI422023 | Hs.161338 | ESTs | 298.6 | 16.6 |
| 314096 | AW977642 | Hs.291742 | ESTs | 165.6 | 16.6 |
| 338728 | | | predicted exon | 165.4 | 16.5 |
| 316609 | AW292520 | Hs.122082 | ESTs | 165 | 16.5 |
| 305989 | AA888220 | | gb.oj15h01.s1 NCI_CGAP_Kid5 Homo sapiens | 164.6 | 16.5 |
| 312642 | AW052128 | | gb:wx26c02.x1 NCI_CGAP_Kid11 Homo sapien | 164 | 16.4 |
| 339236 | | | predicted exon | 163.6 | 16.4 |
| 317058 | AI217713 | Hs.147586 | ESTs | 161.8 | 16.2 |
| 311137 | AW207582 | Hs.196042 | ESTs | 582.2 | 16.2 |
| 310178 | AI936450 | Hs 147482 | ESTs | 161.2 | 16.1 |
| 320745 | H51696 | Hs.89278 | hypothetical protein FLJ11186 | 161 | 16.1 |
| 317336 | AW014637 | Hs.130212 | ESTs | 160 | 16 |
| 309871 | AW300366 | | gb.xs63b05.x1 NCI_CGAP_Kid11 Homo sapien | 159.8 | 16 |
| 302038 | AC004076 | Hs.129709 | Homo sapiens chromosome 19, cosmid R3021 | 159 | 15.9 |
| 332237 | N52883 | Hs.102676 | EST | 159 | 15.9 |
| 312362 | AW015994 | | gb.UI-H-BI0p-abh-g-09-0-UI.s1 NCI_CGAP_S | 158.6 | 15.9 |
| 331558 | N62401 | Hs 48531 | EST | 158.6 | 15.9 |
| 316215 | AI684535 | Hs.200811 | ESTs | 158.4 | 158 |
| 336059 | | | predicted exon | 157.4 | 15.7 |
| 302790 | AJ245245 | | gb.Homo sapiens mRNA for immunoglobulin | 155.8 | 15.6 |
| 328418 | | | predicted exon | 153.8 | 15.4 |
| 304229 | AK000149 | Hs.29493 | hypothetical protein FLJ20142 | 153.6 | 15.4 |
| 331606 | AW273285 | Hs.50802 | ESTs | 153 | 15.3 |
| 338962 | | | predicted exon | 664.4 | 15.3 |
| 317959 | AI204202 | Hs.130264 | ESTs | 152.6 | 15.3 |
| 336228 | | | predicted exon | 152.4 | 15.2 |
| 313534 | AW072916 | Hs.78743 | zinc finger protein 131 (clone pHZ-10) | 152.2 | 15.2 |
| 317404 | AI806867 | Hs.126594 | ESTs | 152.2 | 15.2 |
| 311943 | AI469911 | Hs.26498 | hypothetical protein FLJ21657 | 152 | 15.2 |
| 314680 | AI247425 | Hs.152182 | ESTs | 151.4 | 15.1 |
| 331484 | N29696 | Hs.44076 | EST | 151.2 | 15.1 |
| 338116 | | | predicted exon | 151.2 | 15.1 |
| 329863 | | | predicted exon | 150.6 | 15.1 |
| 315555 | AW452886 | Hs.239107 | ESTs | 149.6 | 15 |
| 317039 | AA868583 | Hs.126153 | ESTs | 149.6 | 15 |
| 331138 | R63816 | Hs.28445 | ESTs | 149.6 | 15 |
| 316561 | AI917222 | Hs 121655 | ESTs | 149.4 | 14.9 |
| 328695 | | | predicted exon | 149.2 | 14.9 |
| 302282 | BE396283 | Hs 173987 | eukaryotic translation initiation factor | 148.4 | 14.8 |
| 318781 | F11802 | Hs.6818 | ESTs | 148.2 | 14.8 |
| 323709 | AW297246 | Hs.288546 | Homo sapiens cDNA FLJ14190 fis, clone NT | 148 | 14.8 |
| 310790 | AW192063 | Hs 248865 | ESTs | 147.8 | 14.8 |
| 316833 | AW292614 | Hs 124367 | ESTs | 147.8 | 148 |
| 323176 | NM_007350 | Hs.82101 | pleckstrin homology-like domain, family | 229 | 14.8 |
| 324188 | AW274439 | Hs.252709 | ESTs | 147.6 | 14.8 |
| 317441 | AA922798 | Hs.196583 | ESTs | 147.4 | 14.7 |
| 317584 | AI825890 | Hs.220513 | ESTs | 146.8 | 14.7 |
| 321798 | AI308206 | Hs.181959 | ESTs | 146.8 | 14.7 |
| 304363 | AA206045 | | gb:zq77f05.s1 Stratagene hNT neuron (937 | 146.6 | 14.7 |
| 313952 | F20956 | | gb:HSPD05390 HM3 Homo sapiens cDNA clone | 146.6 | 14.7 |
| 301909 | AI702609 | Hs.15713 | ESTs | 263.8 | 14.7 |
| 309196 | AI904895 | Hs 9614 | nucleophosmin (nucleolar phosphoprotein | 146.2 | 14.6 |
| 321860 | N47474 | Hs 212631 | ESTs | 146.2 | 14.6 |
| 330187 | | | predicted exon | 146 | 14.6 |
| 323042 | AA463571 | Hs 172550 | polypyrimidine tract binding protein (he | 145.6 | 14.6 |
| 313636 | AA262397 | Hs.201366 | ESTs | 145.2 | 14.5 |
| 302437 | AB024729 | Hs.227473 | UDP-N-acetylglucosamine:a-1,3-D-mannosid | 145 | 14.5 |
| 318197 | AI473096 | Hs.133403 | ESTs | 144.8 | 145 |
| 302749 | M16951 | | gb.Human Ig mu-chain mRNA VDJ4-region, 5 | 144.6 | 14.5 |
| 322357 | AI734258 | Hs 245367 | ESTs, Weakly similar to ALU1_HUMAN ALU S | 144.6 | 14.5 |
| 300391 | AI927371 | Hs.288839 | hypothetical protein FLJ12178 | 144.4 | 14.4 |
| 326077 | | | predicted exon | 144.4 | 14.4 |
| 302004 | Y18264 | Hs.123094 | sal (Drosophila)-like 1 | 144 | 14.4 |
| 320668 | AA805666 | Hs.146217 | Homo sapiens cDNA: FLJ23077 fis, clone L | 144 | 14.4 |
| 331212 | T88693 | Hs.226410 | ESTs | 144 | 14.4 |
| 311268 | AI969727 | Hs.231859 | ESTs | 143.2 | 14.3 |
| 305159 | AA659166 | Hs.275668 | EST,WeaklysimilartoEF1D_HUMANELONGATIONF | 143 | 14.3 |
| 304510 | AA457391 | Hs.119122 | ribosomalproteinL13a | 142.8 | 14.3 |
| 320852 | AA772920 | Hs.303527 | ESTs | 142.8 | 14.3 |
| 330854 | AW291944 | Hs.122139 | ESTs | 142.8 | 14.3 |
| 318275 | AW449952 | Hs.190125 | basic-helix-loop-helix-PAS protein | 142.6 | 14.3 |
| 314992 | AI824879 | Hs.211286 | ESTs, Weakly similar to 1207289A reverse | 142.2 | 14.2 |
| 322631 | AA001697 | Hs.293565 | ESTs, Weakly similar to putative p150 [H | 142.2 | 14.2 |
| 332283 | R40855 | Hs.100839 | EST | 142 | 14.2 |
| 302894 | AA719572 | Hs 274441 | Homo sapiens mRNA; cDNA DKFZp434N011 (fr | 141.2 | 14.1 |
| 301808 | R35391 | Hs.252831 | reticulon 3 | 141 | 14.1 |
| 318608 | AI204491 | Hs.151502 | ESTs | 141 | 14.1 |
| 316499 | AW292947 | Hs.122872 | ESTs | 140.8 | 14.1 |
| 317011 | AI248760 | Hs 150276 | ESTs | 140.8 | 14.1 |
| 321840 | N45600 | Hs.46534 | Homo sapiens mRNA; cDNA DKFZp434P0714 (f | 140.8 | 14.1 |
| 327365 | | | predicted exon | 140.8 | 14.1 |
| 331264 | AA278898 | Hs.225979 | hypothetical protein similar to small G | 140.8 | 14.1 |
| 324545 | AW501944 | Hs.127243 | Homo sapiens mRNA for KIAA1724 protein, | 140.4 | 14 |
| 312986 | AA211586 | | gb.zn56d05.s1 Stratagene muscle 937209 H | 140.2 | 14 |
| 316053 | AA825814 | Hs.149065 | ESTs | 140.2 | 14 |
| 330723 | BE247449 | Hs.31082 | hypothetical protein FLJ10525 | 140.2 | 14 |
| 304876 | AA595765 | | gb:nj28g06.s1 NCI_CGAP_AA1 Homo sapiens | 139.8 | 14 |
| 311379 | AW134766 | Hs.202450 | ESTs | 139.8 | 14 |
| 318265 | AW019873 | Hs 146840 | ESTs | 139.8 | 14 |
| 324137 | AA393127 | Hs.222762 | ESTs | 139.8 | 14 |
| 328262 | | | predicted exon | 139.6 | 14 |
| 322349 | AK001279 | Hs.180171 | Homo sapiens cDNA FLJ10417 fis, clone NT | 139.4 | 13.9 |
| 323504 | AA280223 | Hs.130865 | ESTs | 139.4 | 13.9 |
| 304261 | AA059387 | | gb.zf66d01.s1 Soares retina N2b4HR Homo | 139.2 | 13.9 |
| 310489 | AW451493 | Hs.235516 | hypothetical protein PRO2955 | 139.2 | 13.9 |
| 335946 | | | predicted exon | 139.2 | 13.9 |
| 318155 | AI041546 | Hs.132133 | ESTs | 138.8 | 13.9 |
| 313796 | AI797169 | Hs 208486 | ESTs | 138.6 | 13.9 |
| 333977 | | | predicted exon | 138.6 | 13.9 |
| 324845 | AW969635 | Hs.283718 | ESTs | 138.2 | 13.8 |
| 331139 | R65706 | | gb:yi16g12.s1 Soares placenta Nb2HP Homo | 138.2 | 13.8 |
| 331131 | R54797 | | gb:yg87b07.s1 Soares infant brain 1NIB H | 669.6 | 13.8 |
| 321250 | H58539 | Hs.151692 | ESTs | 138 | 13.8 |
| 312498 | AA668782 | Hs.191284 | ESTs, Weakly similar to ALU1_HUMAN ALU S | 137.8 | 13.8 |
| 331252 | W52470 | Hs 34578 | alpha2,3-sialyltransferase | 137.8 | 138 |
| 337407 | | | predicted exon | 137.8 | 13.8 |
| 303973 | AW512014 | | gb:xx68a03.x1 NCI_CGAP_Lym12 Homo sapien | 137.4 | 13.7 |
| 314582 | AA412258 | Hs.188817 | ESTs | 137.4 | 13.7 |
| 327373 | | | predicted exon | 137.2 | 13.7 |
| 323367 | AA234591 | Hs.304123 | ESTs | 136.6 | 13.7 |
| 316207 | AA832065 | Hs.120260 | ESTs | 136.4 | 13.6 |
| 315231 | AA705809 | Hs.119922 | ESTs | 136.2 | 13.6 |
| 318592 | T39310 | Hs.1139 | cold shock domain protein A | 136.2 | 13.6 |
| 320906 | AW969706 | Hs 293332 | ESTs | 136.2 | 13.6 |
| 328937 | | | predicted exon | 136.2 | 13.6 |
| 329073 | | | predicted exon | 136.2 | 13.6 |
| 318231 | AV659082 | Hs.134228 | ESTs | 136 | 13.6 |
| 311992 | AL360200 | Hs 114145 | ESTs | 135.8 | 13.6 |
| 316497 | AA766457 | Hs 136849 | ESTs | 135.8 | 13.6 |
| 317677 | AA968594 | Hs.127868 | ESTs | 135.8 | 13.6 |
| 321680 | W02848 | Hs.93704 | ESTs | 135.8 | 13.6 |
| 326080 | | | predicted exon | 135.8 | 13.6 |
| 330938 | AF036943 | Hs.172619 | KIAA1106 protein | 135.8 | 13.6 |
| 306573 | AL134878 | Hs 119500 | nbosomal protein, large P2 | 135.6 | 13.6 |
| 307383 | AI223207 | Hs.147888 | EST | 135.6 | 13.6 |
| 311114 | AW449382 | Hs.195297 | ESTs | 135.6 | 13.6 |
| 320579 | R15138 | Hs.165570 | Homo sapiens clone 25052 mRNA sequence | 135 | 13.5 |
| 301328 | AA884104 | Hs.125546 | ESTs | 134.8 | 13.5 |
| 312063 | N58198 | Hs.182898 | ESTs | 134.8 | 13.5 |
| 323036 | H09604 | Hs 13268 | ESTs | 134.6 | 13.5 |
| 332776 | AF241850 | Hs 151428 | ret finger protein 2 | 134.4 | 13.4 |
| 332494 | AA282330 | Hs.145668 | ESTs | 134.2 | 13.4 |
| 334376 | | | predicted exon | 134.2 | 13.4 |
| 313264 | N93416 | Hs 118228 | ESTs | 133.6 | 13.4 |
| 313669 | AA351109 | Hs.5437 | Taxi (human T-cell leukemia virus type I | 133.2 | 13.3 |
| 312083 | T87398 | Hs.205816 | ESTs | 132.6 | 13.3 |
| 319354 | AA993807 | Hs.167367 | ESTs | 132.6 | 13.3 |
| 307414 | AI242106 | | gb:qh92a02.x1 Soares_NFL_T_GBC_S1 Homo s | 132.2 | 13.2 |
| 312771 | AA018515 | Hs.264482 | Apg12 (autophagy 12, S. cerevisiae)-like | 131.8 | 13.2 |
| 313004 | AI274963 | Hs.145900 | ESTs | 131.2 | 13.1 |
| 300995 | AW510641 | Hs.258018 | ESTs | 220.6 | 13 |
| 319323 | F12650 | Hs 13287 | ESTs | 125.4 | 12.5 |
| 329451 | | | predicted exon | 123.4 | 12.3 |
| 337603 | | | predicted exon | 572 | 12.2 |
| 312480 | R68651 | Hs.144997 | ESTs | 121.4 | 12.1 |
| 324934 | AW452051 | Hs 147546 | ESTs | 119.4 | 11.9 |
| 320723 | BE178025 | Hs 7942 | hypothetical protein FLJ20080 | 117 | 11.7 |
| 318188 | AI792566 | | gb:qi74f02.y5 NCI_CGAP_Ov26 Homo sapiens | 116.6 | 11.7 |
| 320873 | AF238869 | Hs 283955 | Homo sapiens clone GLSH-2 similar to gli | 112.8 | 11.3 |
| 331005 | BE003191 | Hs.119555 | ESTs | 112.6 | 11.3 |
| 304969 | AA614406 | | gb:np46f05.s1 NCI_CGAP_Br11 Homo sapiens | 112.4 | 11.2 |
| 319799 | AI139253 | Hs.227767 | zinc finger protein 41 | 111.2 | 11.1 |
| 302610 | AA347945 | Hs.256024 | ESTs | 111 | 11.1 |
| 309485 | AW130320 | Hs.108124 | ribosomalproteinS4,X-linked | 111 | 11.1 |
| 311880 | AW419225 | Hs.256247 | ESTs | 110.2 | 11 |
| 313981 | AW452334 | Hs.128148 | ESTs | 110.2 | 11 |
| 322442 | W49701 | Hs 29667 | ESTs | 109.4 | 10.9 |
| 315099 | AA806536 | Hs 291841 | ESTs | 109 | 10.9 |
| 304793 | AA583264 | Hs 182979 | nbosomalproteinL12 | 108.8 | 10.9 |
| 330815 | AA019211 | Hs.236463 | KIAA1238 protein | 108.8 | 10.9 |
| 304044 | T81656 | Hs.252259 | ribosomal protein S3 | 714.8 | 10.8 |
| 325222 | | | predicted exon | 135 | 10.8 |
| 325889 | | | predicted exon | 814.6 | 10.8 |
| 321447 | AW891130 | Hs.38173 | ESTs | 107.8 | 10.8 |
| 302990 | AA496212 | Hs 180182 | ESTs | 106.2 | 10.6 |
| 308106 | AI476803 | | gb.tj77e12.x1 Soares_NSF_F8_9W_OT_PA_P_S | 270.6 | 10.6 |
| 310536 | AI301041 | Hs.150174 | ESTs | 106 | 10.6 |
| 315257 | AW157431 | Hs 248941 | ESTs | 233 | 10.6 |
| 318787 | Z42313 | Hs 22657 | ESTs | 105.8 | 10.6 |
| 312306 | AI927226 | Hs.175610 | ESTs | 105.2 | 10.5 |
| 326788 | | | predicted exon | 104.4 | 10.4 |
| 312234 | AA830640 | Hs.206934 | ESTs | 104 | 10.4 |
| 314482 | AW085525 | Hs.134182 | ESTs | 234 | 10.4 |
| 323597 | AI185693 | Hs 135119 | ESTs | 102.4 | 10.2 |
| 302623 | AW836724 | Hs.194110 | hypothetical protein PRO2730 | 162.4 | 10.2 |
| 323594 | AI791531 | Hs.129993 | ESTs | 101 | 10.1 |
| 324315 | N55761 | Hs.194718 | zinc finger protein 265 | 100.2 | 10 |
| 314217 | AA256465 | Hs.188725 | ESTs | 99.2 | 9.9 |
| 320932 | AA554913 | Hs.162297 | ESTs | 98.2 | 9.8 |
| 327876 | | | predicted exon | 98.2 | 9.8 |
| 319736 | R17424 | Hs.6650 | vacuolar protein sorting 45B (yeast homo | 98 | 9.8 |
| 327747 | | | predicted exon | 97.6 | 9.8 |
| 327844 | | | predicted exon | 97.4 | 9.7 |
| 318200 | AI061192 | Hs.166517 | ESTs | 97.2 | 9.7 |
| 329414 | | | predicted exon | 97.2 | 9.7 |
| 318296 | AI089667 | Hs 270713 | ESTs | 121.4 | 9.7 |
| 307010 | AI140014 | | gb:qa68f09.x1 Soares_fetal_heart_NbHH19W | 295 | 9.7 |
| 319792 | AI138635 | Hs.22968 | ESTs | 385.4 | 9.6 |
| 305671 | AA811688 | Hs 82113 | dUTPpyrophosphatase | 96 | 9.6 |
| 329440 | | | predicted exon | 93.8 | 9.4 |
| 310381 | AI263059 | Hs.145594 | ESTs | 93.4 | 9.3 |
| 318824 | F06771 | Hs.27226 | ESTs | 93.4 | 9.3 |
| 328957 | | | predicted exon | 92.2 | 9.2 |
| 318804 | Z42549 | Hs.160893 | ESTs | 92 | 9.2 |
| 330836 | AA055611 | Hs.226568 | ESTs, Moderately similar to ALU4_HUMAN A | 92 | 9.2 |
| 324592 | AW752437 | Hs.325708 | ESTs | 91.8 | 9.2 |
| 311820 | AW274545 | Hs.254333 | ESTs | 91.4 | 9.1 |
| 321614 | H86161 | | gb:ys94b01.r1 Soares retina N2b5HR Homo | 91 | 9.1 |
| 330306 | | | predicted exon | 91 | 9.1 |
| 303096 | AL080276 | Hs.268562 | regulator of G-protein signalling 17 | 90 | 9 |
| 313275 | AI027604 | Hs.159650 | ESTs | 110.4 | 8.8 |
| 302593 | H54855 | Hs.36958 | ESTs | 88 | 8.8 |
| 321421 | BE465115 | Hs.171688 | ESTs | 86.2 | 8.6 |
| 330832 | AI133530 | Hs.62930 | ESTs | 456.4 | 8.6 |
| 311847 | AW301807 | Hs.297260 | ESTs | 86 | 8.6 |
| 322036 | BE002723 | Hs.301905 | Homo sapiens cDNA FLJ14080 fis, clone HE | 145.8 | 8.6 |
| 328688 | | | predicted exon | 85.6 | 8.6 |
| 325251 | | | predicted exon | 85.4 | 8.5 |
| 329088 | | | predicted exon | 85.4 | 8.5 |
| 322524 | W79027 | Hs.271762 | ESTs | 84 | 8.4 |
| 337953 | | | predicted exon | 451 | 8.3 |
| 323529 | AA284397 | Hs.201485 | Homo sapiens clone FLC0664 PRO2866 mRNA, | 82.6 | 8.3 |
| 307041 | AI144243 | | gb.qb85b12x1 Soares_fetal_heart_NbHH19W | 306.8 | 8.2 |
| 318285 | AI332454 | Hs.158412 | ESTs | 81.4 | 8.1 |
| 312021 | AA759263 | Hs 14041 | ESTs | 81 | 8.1 |
| 329350 | | | predicted exon | 81 | 8.1 |
| 326169 | | | predicted exon | 80.4 | 8 |
| 338038 | | | predicted exon | 1024.2 | 7.9 |
| 312549 | AI214510 | Hs.146304 | ESTs | 77.4 | 7.7 |
| 312542 | D60076 | | gb:HUMO84E10A Clontech human fetal brain | 76.8 | 7.7 |
| 320992 | AB026891 | Hs 225972 | solute carrier family 7, (cationic amino | 76 | 7.6 |
| 318596 | AI470235 | Hs 172698 | EST | 150.6 | 7.5 |
| 315650 | AA649042 | Hs.269615 | ESTs | 73.4 | 7.3 |
| 324328 | AA447276 | Hs.292020 | ESTs | 210.4 | 7.1 |
| 332622 | R10674 | Hs 128856 | CSR1 protein | 70.2 | 7 |
| 328229 | | | predicted exon | 69.4 | 6.9 |
| 319110 | T75260 | Hs.98321 | hypothetical protein FLJ14103 | 68.6 | 6.9 |
| 316133 | AI187742 | Hs 125562 | ESTs | 308.6 | 6.9 |
| 303992 | AW515800 | | gb hd88g01 x1 NCI_CGAP_GC6 Homo sapiens | 67.8 | 6.8 |
| 322675 | AA017656 | Hs.146580 | enolase 2, (gamma, neuronal) | 377.2 | 6.7 |
| 325753 | | | predicted exon | 105.2 | 6.6 |
| 312539 | AI004377 | Hs 200360 | Homo sapiens cDNA FLJ13027 fis, clone NT | 92.2 | 6.4 |
| 302592 | AA294921 | Hs.250811 | v-ral simian leukemia viral oncogene hom | 361.6 | 6.3 |
| 314578 | AA410183 | Hs.137475 | ESTs | 201.6 | 6.1 |
| 335986 | | | predicted exon | 108.6 | 6 |
| 321478 | AW402593 | Hs.123253 | hypothetical protein FLJ22009 | 528 | 6 |
| 305192 | AA666019 | | gb:ag44a04 s1 Jia bone marrow stroma Hom | 58.6 | 5.9 |
| 304275 | AA070605 | | gb:zm53h09.s1 Stratagene fibroblast (937 | 78.6 | 5.6 |
| 302779 | AJ235667 | | gb:Homo sapiens mRNA for immunoglobulin | 278.8 | 5.5 |
| 301976 | T97905 | Hs.77256 | enhancer of zeste (Drosophila) homolog 2 | 479.2 | 5.4 |
| 316021 | AW293399 | Hs.144904 | nuclear receptor co-repressor 1 | 792.4 | 5.3 |
| 320802 | 8E336699 | Hs.185055 | BENE protein | 2423.8 | 5.3 |
| 317282 | AI733112 | Hs.176101 | ESTs | 523.2 | 5.1 |
| 316827 | AI380429 | Hs.172445 | ESTs | 578 | 5.1 |
| 303190 | BE280787 | Hs.16079 | hypothetical protein FLJ10233 | 223 | 5.1 |
| 315587 | AI268399 | Hs.140489 | ESTs | 136.2 | 5 |
| 333122 | | | predicted exon | 399 | 5 |
| 310214 | AI220072 | Hs.165893 | ESTs | 234.4 | 4.9 |
| 320089 | D43945 | Hs.113274 | transcription factor EC | 68 | 4.9 |
| 309328 | AW024348 | Hs.233191 | EST, Weakly similar to A27217 glucose tr | 258.8 | 4.8 |
| 318971 | Z44067 | Hs.10957 | ESTs | 376.6 | 4.8 |
| 327220 | | | predicted exon | 47.4 | 4.7 |
| 315757 | AW014605 | Hs.179872 | ESTs | 177.4 | 4.7 |
| 320730 | R68869 | Hs.151072 | ESTs | 205.2 | 4.6 |
| 313339 | AI682536 | Hs.163495 | Homo sapiens cDNA FLJ13608 fis, clone PL | 260 | 4.5 |
| 318634 | T49598 | Hs.156832 | ESTs | 475.2 | 4.5 |
| 320955 | AW820035 | Hs.278679 | a disintegrin and metalloproteinase doma | 388.6 | 4.4 |
| 306605 | AI000497 | Hs.119500 | ribosomalprotein,largeP2 | 81.6 | 4.4 |
| 309349 | AW051913 | | gb.wx24a09 x1 NCI_CGAP_Kid11 Homo sapien | 102.4 | 4.3 |
| 306004 | AA889992 | Hs.2186 | eukaryotictranslationelongationfactor1ga | 451.2 | 4.2 |
| 330020 | | | predicted exon | 61.2 | 4.1 |
| 302308 | AW327279 | Hs.91379 | ribosomal protein L26 | 342 | 3.9 |
| 314648 | AW979268 | | gb:EST391378 MAGE resequences, MAGP Homo | 56.4 | 3.8 |
| 315131 | AI753709 | Hs.152484 | ESTs | 130.4 | 3.7 |
| 313690 | AI493591 | Hs.78146 | platelet/endothelial cell adhesion molec | 3179.6 | 3.6 |
| 333585 | | | predicted exon | 175.4 | 3.5 |
| 312911 | H93366 | Hs.7567 | Homo sapiens cDNA: FLJ21962 fis, clone H | 219 | 3.5 |
| 322966 | AA633669 | Hs.235920 | Homo sapiens cell recognition molecule C | 350.2 | 3.4 |
| 312492 | R71072 | Hs 191269 | ESTs | 322.8 | 3 |
| 318988 | Z44203 | Hs.26418 | ESTs | 25 | 2.5 |
| 332363 | AI123705 | Hs.106932 | ESTs | 773.4 | 2.5 |
| 324181 | AI025476 | Hs.131628 | ESTs | 634.8 | 2.4 |
| 311717 | AW205369 | Hs.312830 | ESTs | 54.2 | 2.4 |
| 321342 | AA127984 | Hs 222024 | transcription factor BMAL2 | 23.4 | 2.3 |
| 308852 | AI829848 | Hs 182937 | peptidylprolylisomeraseA(cyclophilinA) | 92 | 2.3 |
| 331466 | AA373210 | Hs.43047 | Homo sapiens cDNA FLJ13585 fis, clone PL | 494 | 2.3 |
| 320279 | AB033062 | Hs 134970 | DKFZP434N178 protein | 76.2 | 2.2 |
| 322221 | N24236 | Hs.179662 | nucleosome assembly protein 1-like 1 | 253.2 | 2.1 |
| 302925 | AL137449 | Hs.126666 | homeo box 84 | 136.6 | 2.1 |
| 331384 | AB041035 | Hs.93847 | NADPH oxidase 4 | 720 | 1.8 |
| 300938 | AA514416 | Hs.152320 | ESTs, Weakly similar to 1605244A erythro | 27 | 1.8 |
| 312695 | AW196663 | Hs 200242 | ESTs | 303.8 | 1.6 |
| 320223 | W35132 | Hs 267442 | ESTs | 189 | 1.5 |
| 332743 | AW247977 | Hs.87595 | translocase of inner mitochondrial membr | 14.4 | 1.4 |
| 331039 | AW378685 | Hs.18625 | Mitochondrial Acyl-CoA Thioesterase | 529.8 | 1.4 |
| 333123 | | | predicted exon | 396.2 | 1.4 |
| 328455 | | | predicted exon | 91.8 | 1.3 |
| 334458 | | | predicted exon | 406.4 | 1.3 |
| 313478 | AA643008 | Hs.192775 | ESTs | 413.4 | 1.1 |
| 309899 | AW338564 | Hs.217493 | annexinA2 | −30.8 | 1 |
| 311735 | AW294416 | Hs.144687 | Homo sapiens cDNA FLJ12981 fis, clone NT | −62.8 | 1 |
| 312953 | NM_001992 | Hs 128087 | coagulation factor II (thrombin) recepto | −73.6 | 1 |
| 313055 | AW367295 | Hs.241175 | ESTs | −438 | 1 |
| 313291 | AI267970 | Hs.150614 | ESTs, Weakly similar to ALU4_HUMAN ALU S | −63 | 1 |
| 315059 | AW275110 | Hs.271106 | ESTs | −67 | 1 |
| 322284 | AI792140 | Hs.49265 | ESTs | −395.2 | 1 |
| 322450 | AL121278 | Hs.25144 | ESTs | −1.6 | 1 |
| 324803 | AW975183 | Hs.292663 | ESTs | 4.4 | 1 |
| 331495 | AW970939 | Hs.291039 | ESTs | −282.8 | 1 |
| 333610 | | | predicted exon | −152.6 | 1 |
| 335093 | | | predicted exon | −23.2 | 1 |
| 339403 | | | predicted exon | −331.2 | 1 |
| 302820 | X04588 | Hs.85844 | neurotrophic tyrosine kinase, receptor, | 591.2 | 1 |
| 302270 | R56151 | Hs.93589 | Homo sapiens mRNA; cDNA DKFZp564B1162 (f | 276.6 | 1 |
| 323755 | AW300094 | Hs 136252 | ESTs | 135 | 0.9 |
| 326946 | | | predicted exon | 727.4 | 0.9 |
| 315343 | BE144306 | Hs.179891 | ESTs, Weakly similar to P4HA_HUMAN PROLY | 122.8 | 0.9 |
| 311168 | AK001270 | Hs 196086 | hypothetical protein FLJ10408 | 304 | 0.9 |
| 329732 | | | predicted exon | 109.2 | 0.9 |
| 321415 | BE621807 | Hs 3337 | transmembrane 4 superfamily member 1 | 414.8 | 0.7 |
| 333121 | | | predicted exon | 87.8 | 0.7 |
| 333120 | | | predicted exon | 379.8 | 0.7 |
| 330392 | AW797956 | Hs.75748 | proteasome (prosome, macropain) subunit, | 589.2 | 0.7 |
| 314711 | AA769365 | Hs 126058 | ESTs | −87 | 0.6 |
| 330865 | BE409857 | Hs.69499 | hypothetical protein | 347.4 | 0.6 |
| 333169 | | | predicted exon | −1182 | 0.6 |
| 335095 | | | predicted exon | 106.4 | 0.6 |
| 335815 | | | predicted exon | −156 | 0.6 |
| 330232 | | | predicted exon | 102.6 | 0.6 |
| 330823 | AA031565 | Hs.221255 | ESTs, Moderately similar to ALU5_HUMAN A | −62 | 0.5 |
| 331704 | F04225 | Hs.66032 | ESTs | −14.6 | 0.5 |
| 302642 | NM_016428 | Hs.130719 | NESH protein | 267.6 | 0.5 |
| 304484 | AA432067 | Hs.258373 | ESTs | 85 | 0.5 |
| 310230 | AK000377 | Hs.144840 | homolog of mouse C2PA | −70 | 0.4 |
| 301531 | AI077462 | Hs.134084 | ESTs | −195.4 | 0.4 |
| 306337 | AA954221 | Hs.73742 | nbosomalprotein,large,PO | −33.4 | 0.4 |
| 331327 | N46436 | Hs.109221 | ESTs | −392 | 0.4 |
| 332961 | | | predicted exon | −5.6 | 0.4 |
| 322796 | W31178 | Hs.154140 | Homo sapiens ovary-specific acidic prote | −880.6 | 0.3 |
| 328857 | | | predicted exon | 55.2 | 0.3 |
| 316342 | AA743935 | Hs.202329 | ESTs | 43.4 | 0.3 |
| 331263 | AW780192 | Hs.267596 | ESTs | −180.4 | 0.3 |
| 335987 | | | predicted exon | −134 | 0.3 |
| 311923 | T60843 | Hs.189679 | ESTs | 12.2 | 0.3 |
| 310522 | AW134529 | Hs 244647 | ESTs | −187.8 | 0.3 |
| 315363 | AA759190 | Hs.121454 | ESTs, Weakly similar to olfactory recept | 80 | 0.3 |
| 302032 | NM_001992 | Hs.128087 | coagulation factor II (thrombin) recepto | −877 | 0.3 |
| 313140 | BE265133 | Hs.217493 | annexin A2 | 95.4 | 0.3 |
| 310860 | AW015920 | Hs.161359 | ESTs | −239 | 0.3 |
| 317899 | AI952430 | Hs.150614 | ESTs, Weakly similar to ALU4_HUMAN ALU S | −715.2 | 0.3 |
| 328520 | | | predicted exon | −109.2 | 0.2 |
| 302406 | NM_012099 | Hs.211956 | CD3-epsilon-associated protein; antisens | 10 | 0.2 |
| 311804 | AI866921 | Hs.203349 | Homo sapiens cDNA FLJ12149 fis, clone MA | −252.6 | 0.2 |
| 315065 | AK001122 | Hs.105859 | hypothetical protein FLJ10260 | −46.2 | 0.2 |
| 314129 | AA228366 | Hs.115122 | ESTs | −308.8 | 0.2 |
| 335697 | | | predicted exon | −47.2 | 0.2 |
| 335989 | | | predicted exon | 89 | 0.2 |
| 320606 | AW867943 | Hs.127216 | hypothetical protein FLJ13465 | −205.6 | 0.2 |
| 329745 | | | predicted exon | 103 | 0.2 |
| 313628 | AW419069 | Hs.209670 | ESTs | −177.8 | 0.2 |
| 334616 | | | predicted exon | −936.6 | 0.2 |
| 308820 | AI821267 | Hs.207243 | EST | −1.2 | 0.2 |
| 320416 | AI026984 | Hs.293662 | ESTs | −18.4 | 0.2 |
| 335211 | | | predicted exon | −142 | 0.2 |
| 323629 | AA375957 | Hs.6682 | ESTs | −100 | 0.1 |
| 331420 | AW452904 | | gb:UI-H-BI3-aly-h-11-0-UI.s1 NCI_CGAP_Su | 83 | 0.1 |
| 315984 | AI015862 | Hs.131793 | ESTs | −250.6 | 0.1 |
| 332833 | | | predicted exon | −374.2 | 0.1 |
| 332607 | NM_002314 | Hs.36566 | LIM domain kinase 1 | −27.6 | 0.1 |
| 313467 | AA004879 | Hs.187820 | ESTs | −288.2 | 0.1 |
| 323333 | AV651680 | Hs.208558 | ESTs | −735.6 | 0.1 |
| 330775 | AW247020 | Hs.250747 | SUMO-1 activating enzyme subunit 1 | 53.6 | 0.1 |
| 333168 | | | predicted exon | −1041.8 | 0.1 |
| 332079 | AI308876 | Hs.103849 | ESTs | 19.4 | 0.1 |
| 322724 | AF161442 | Hs 191591 | Homo sapiens HSPC324 mRNA, partial cds | −123.6 | 0.1 |
| 303652 | AI799111 | Hs.64341 | ESTs | −46.4 | 0.1 |
| 303131 | AW081061 | Hs.103180 | DC2 protein | −156.4 | 0.1 |
| 320716 | AI479439 | Hs.171532 | ESTs | −146.6 | 0.1 |
| 300454 | AA659037 | Hs.163780 | ESTs | −304 | 0.1 |
| 312757 | AI285970 | Hs 183817 | ESTs | −445 | 0.1 |
| 312391 | R43707 | Hs.133159 | ESTs, Weakly similar to PIHUSD salivary | −111.8 | 0.1 |
| 308877 | AI832519 | | gb:at69h03.x1 Barstead colon HPLRB7 Homo | −149.6 | 0 |
| 311275 | AI659166 | Hs.207144 | ESTs | −62.6 | 0 |
| 302363 | AW163799 | Hs.198365 | 2,3-bisphosphoglycerate mutase | −15 | 0 |
| 321717 | AW956580 | Hs.42699 | ESTs | −1059.6 | 0 |
| 302638 | AA463798 | Hs.102696 | MCT-1 protein | −332.2 | 0 |
| 306352 | AA961367 | | gb:or52a05.s1 NCI_CGAP_GC3 Homo sapiens | 21.8 | 0 |
| 313798 | AI292148 | Hs.71622 | SWI/SNF related, matrix associated, acti | −97.2 | 0 |
| 320807 | AA135370 | Hs.188536 | Homo sapiens cDNA.FLJ21635 fis, clone C | −2222 | 0 |
| 320931 | AW262836 | Hs.252844 | ESTs | −881.6 | 0 |
| 332450 | AW288085 | Hs.11156 | hypothetical protein | 28.4 | 0 |
| 332535 | AF167706 | Hs 19280 | cysteine-rich motor neuron 1 | −722 | 0 |
| 335990 | | | predicted exon | −421 | 0 |
| 330746 | A8033888 | Hs 8619 | SRY (sex determining region Y)-box 18 | 35.4 | 0 |
| 316820 | AI627912 | Hs.130783 | Forssman synthetase | −373.6 | 0 |
| 337429 | | | predicted exon | −257 | 0 |
| 331192 | BE622021 | Hs.152571 | ESTs, Highly similar to IGF-II mRNA-bind | −33 | 0 |
| 330609 | AI346201 | Hs.76118 | ubiquitin carboxyl-terminal esterase L1 | −280 | 0 |
| 323593 | AI739435 | Hs.39168 | ESTs | −3627.6 | 0 |
| 302704 | AA531133 | Hs.4253 | hypothetical protein MGC2574 | −278.6 | 0 |
| 330534 | NM_004579 | Hs.82979 | mitogen-activating protein kinase kinase | −244 | 0 |
| 332374 | X91195 | Hs.100623 | phospholipase C, beta 3, neighbor pseudo | −1204.2 | 0 |
| 333221 | | | predicted exon | −189.6 | 0 |
| 335988 | | | predicted exon | −122.6 | 0 |
| 330574 | AI984144 | Hs 66713 | hepatitis delta antigen-interacting prot | −2257.4 | 0 |
| 312052 | BE621697 | Hs.14317 | nucleolar protein family A, member 3 (H/ | −359.2 | 0 |
| 319568 | AF131781 | Hs.84753 | hypothetical protein FLJ12442 | −874.6 | 0 |
| 337113 | | | predicted exon | −24.6 | 0 |
| 335149 | | | predicted exon | −191.8 | 0 |

TABLE 6A

| Pkey | CAT Number | Accession |
|---|---|---|
| 320925 | 1525201_1 | D62892 D79755 D62760 |
| 321614 | 87866_1 | H86161 AA054308 AA018955 |
| 313952 | 136885_1 | F20956 AA129374 AA133740 AW819878 |
| 314648 | 293660_1 | AW979268 AA878419 AA431342 AA431628 |
| 302749 | 458_107 | M16951 M16952 M16948 M16949 M16950 |
| 312362 | 764066_1 | AW015994 R39898 AW000978 AI598202 AI521706 |
| 312542 | 1522649_1 | D60076 D60259 D61037 |
| 312642 | 1005225_1 | AW052128 H51439 H51481 |
| 312986 | 171879_1 | AA211586 F35799 AA211641 F29720 AW937387 AW937408 |
| 329350 | c_x_hs | |
| 329414 | c_y_hs | |
| 329440 | c_y_hs | |
| 329451 | c_y_hs | |
| 338033 | CH22_6528FG__LINK_EM:AC00 | |
| 338038 | CH22_6535FG__LINK_EM:AC00 | |
| 338116 | CH22_6650FG__LINK_EM:AC00 | |
| 338158 | CH22_6700FG__LINK_EM:AC00 | |
| 329732 | c14_p2 | |
| 329745 | c14_p2 | |
| 308106 | AI476803 | |
| 329863 | c14_p2 | |
| 338316 | CH22_6944FG__LINK_EM:AC00 | |
| 308248 | AI560919 | |
| 338388 | CH22_7034FG__LINK_EM:AC00 | |
| 338442 | CH22_7109FG__LINK_EM:AC00 | |
| 338645 | CH22_7410FG__LINK_EM:AC00 | |
| 338728 | CH22_7527FG__LINK_EM:AC00 | |
| 308877 | AI832519 | |
| 338962 | CH22_7838FG__LINK_DJ32I10 | |
| 308886 | AI833240 | |
| 333120 | CH22_349FG_81_3_LINK_EM:A | |
| 333121 | CH22_350FG_81_4_LINK_EM:A | |
| 333122 | CH22_351FG_81_6_LINK_EM:A | |
| 333123 | CH22_352FG_81_7_LINK_EM:A | |
| 333168 | CH22_400FG_94_1_LINK_EM:A | |
| 333169 | CH22_401FG_94_2_LINK_EM:A | |
| 333221 | CH22_458FG_105_1_LINK_EM: | |
| 326077 | c17_hs | |
| 326080 | c17_hs | |
| 326169 | c17_hs | |
| 326198 | c17_hs | |
| 326230 | c17_hs | |
| 333585 | CH22_846FG_203_4_LINK_EM: | |
| 333610 | CH22_871FG 217_5_LINK_EM: | |
| 335093 | CH22_2423FG_492_3_LINK_EM | |
| 335095 | CH22_2425FG_492_5_LINK_EM | |
| 335149 | CH22_2484FG_499_5_LINK_EM | |
| 326759 | c20_hs | |
| 333977 | CH22_1254FG_309_6_LINK_EM | |
| 326788 | c20_hs | |
| 335211 | CH22_2550FG_511_2_LINK_EM | |
| 305192 | AA666019 | |
| 303973 | AW512014 | |
| 303992 | AW515800 | |
| 326946 | c21_hs | |
| 328229 | c_6_hs | |
| 328262 | c_6_hs | |
| 328418 | c_7_hs | |
| 328455 | c_7_hs | |
| 335697 | CH22_3058FG_596_12_LINK_E | |
| 328520 | c_7_hs | |
| 328548 | c_7_hs | |
| 335815 | CH22_3187FG_618_3_LINK_EM | |
| 328688 | c_7_hs | |
| 328695 | c_7_hs | |
| 307010 | AI140014 | |
| 337113 | CH22_5058FG_493_1_{—} | |
| 307041 | AI144243 | |
| 328700 | c_7_hs | |
| 335946 | CH22_3324FG_646_20_LINK_D | |
| 335986 | CH22_3366FG_654_10_LINK_D | |
| 335987 | CH22_3367FG_654_11_LINK_D | |
| 335988 | CH22_3368FG_654_12_LINK_D | |
| 335989 | CH22_3369FG_655_2_LINK_DJ | |
| 335990 | CH22_3370FG_655_4_LINK_DJ | |
| 337214 | CH22_5288FG_613_7_{—} | |
| 330020 | c16_p2 | |
| 305989 | AA888220 | |
| 328857 | c_7_hs | |
| 328937 | c_8_hs | |
| 328957 | c_8_hs | |
| 330187 | c_4_p2 | |
| 337407 | CH22_5607FG_755_1_{—} | |
| 337429 | CH22_5633FG_762_3_{—} | |
| 330232 | c_5_p2 | |
| 307414 | AI242106 | |
| 330305 | c_7_p2 | |
| 330306 | c_7_p2 | |
| 337603 | CH22_5896FG_LINK_C20H12. | |
| 337953 | CH22_6395FG_LINK_EM:AC00 | |
| 339236 | CH22_8181FG_LINK_BA354I1 | |
| 339403 | CH22_8384FG_LINK_BA232E1 | |
| 309349 | AW051913 | |
| 325222 | c10_hs | |
| 325251 | c10_hs | |
| 318188 | 956161_1 | AI792566 AI053836 AI054127 AI792489 AI288324 |
| 309871 | AW300366 | |
| 325544 | c12_hs | |
| 309931 | AW341683 | |
| 332833 | CH22_50FG_17_7_LINK_C20H1 | |
| 302779 | 33837_1 | AJ235667 AJ235666 AJ235664 AJ235665 AJ235668 AJ235669 AJ235670 |
| 302790 | 34168_1 | AJ245245 AJ245247 AJ245257 AJ245248 AJ245254 AJ245256 AJ245253 |
| | | AJ245203 AJ245250 AJ245252 AJ245243 AJ245204 AJ245201 AJ245206 |
| | | AJ245246 AJ245255 AJ245205 AJ245202 AJ245251 AJ245249 AJ245207 |
| | | AJ245244 |
| 332961 | CH22_185FG_48_18_LINK_EM: | |
| 325753 | c14_hs | |
| 327036 | c21_hs | |
| 325843 | c16_hs | |
| 325889 | c16_hs | |
| 304261 | AA059387 | |
| 304275 | AA070605 | |
| 334376 | CH22_1670FG_379_8_LINK_EM | |
| 327220 | c_1_hs | |
| 304363 | AA206045 | |
| 334458 | CH22_1757FG_391_2_LINK_EM | |
| 327365 | c_1_hs | |
| 327373 | c_2_hs | |
| 334616 | CH22_1923FG_411_15_LINK_E | |
| 327414 | c_2_hs | |
| 327568 | c_3_hs | |
| 336034 | CH22_3419FG_678_5_LINK_DJ | |
| 336059 | CH22_3445FG_684_2_LINK_DJ | |
| 334834 | CH22_2148FG_439_3_LINK_EM | |
| 304782 | AA582081 | |
| 304876 | AA595765 | |
| 327747 | c_5_hs | |
| 336228 | CH22_3626FG_730_4_LINK_DA | |
| 329073 | c_x_hs | |
| 329088 | c_x_hs | |
| 304969 | AA614406 | |
| 327844 | c_5_hs | |
| 327876 | c_6_hs | |
| 306352 | AA961367 | |
| 331131 | genbank_R54797 | R54797 |
| 331139 | Genbank_R65706 | R65706 |
| 331420 | 675963_1 | AW452904 AW449414 BE467906 AI298565 BE549932 BE326357 F04362 |
| Table 6A shows the accession numbers for those pkeys lacking unigeneID's for Table 6. The pkeys in Table 7 lacking unigeneID's are represented within Tables 1-6A. For each prabeset we have listed the gene cluster number from which the oligonucleotides were designed. Gene clusters were compiled using sequences derived from Genbank ESTs and mRNAs These sequences were clustered based on sequence similarity using Clustering and Alignment Tools (DoubleTwist, Oakland California). | | |
| # The Genbank accession numbers for sequences comprising each cluster are listed in the “Accession” column. | | |

Pkey: Unique Eos probeset identifier number
CAT number: Gene cluster number
Accession: Genbank accession numbers

TABLE 6B

| Pkey | Ref | Strand | Nt_position |
|---|---|---|---|
| 332961 | Dunham, I. et al | Plus | 2521424-2521555 |
| 333221 | Dunham, I. et al. | Plus | 3978070-3978187 |
| 333585 | Dunham, I. et al. | Plus | 6234778-6234894 |
| 333610 | Dunham, I. et al | Plus | 6547007-6547116 |
| 334376 | Dunham, I. et al. | Plus | 13902218-13902331 |
| 334458 | Dunham, I. et al. | Plus | 14353496-14353572 |
| 334616 | Dunham, I. et al. | Plus | 15176123-15176470 |
| 335149 | Dunham, I. et al. | Plus | 21497441-21497587 |
| 335211 | Dunham, I. et al. | Plus | 21774611-21774680 |
| 335697 | Dunham, I. et al. | Plus | 25481456-25481649 |
| 335986 | Dunham, I. et al. | Plus | 27967791-27967852 |
| 335987 | Dunham, I. et al. | Plus | 27971413-27971481 |
| 335988 | Dunham, I. et al. | Plus | 27977912-27978013 |
| 335989 | Dunham, I. et al | Plus | 27983788-27983860 |
| 335990 | Dunham, I. et al. | Plus | 27988532-27988608 |
| 336034 | Dunham, I. et al. | Plus | 29014404-29014590 |
| 337953 | Dunham, I. et al. | Plus | 6827029-6827125 |
| 338033 | Dunham, I. et al | Plus | 8092128-8092271 |
| 338038 | Dunham, I. et al | Plus | 8138219-8138392 |
| 338316 | Dunham, I. et al. | Plus | 17089711-17089988 |
| 338442 | Dunham, I. et al. | Plus | 19980640-19980698 |
| 338962 | Dunham, I. et al. | Plus | 29581892-29582020 |
| 332833 | Dunham, I. et al. | Minus | 1119848-1119705 |
| 333120 | Dunham, I. et al. | Minus | 3307508-3307427 |
| 333121 | Dunham, I. et al. | Minus | 3308446-3308358 |
| 333122 | Dunham, I. et al | Minus | 3309596-3309531 |
| 333123 | Dunham, I. et al. | Minus | 3310817-3310749 |
| 333168 | Dunham, I. et al. | Minus | 3729896-3729788 |
| 333169 | Dunham, I. et al. | Minus | 3730864-3730767 |
| 333977 | Dunham, I. et al. | Minus | 8722928-8722725 |
| 334834 | Dunham, I. et al. | Minus | 17182681-17182535 |
| 335093 | Dunham, I. et al. | Minus | 21297367-21297214 |
| 335095 | Dunham, I. et al. | Minus | 21292546-21292381 |
| 335815 | Dunham, I. et al. | Minus | 26320518-26320421 |
| 335946 | Dunham, I. et al. | Minus | 27487203-27487035 |
| 336059 | Dunham, I. et al. | Minus | 29184079-29183969 |
| 336228 | Dunham, I. et al. | Minus | 30904602-30904497 |
| 337113 | Dunham, I. et al. | Minus | 21233344-21233237 |
| 337214 | Dunham, I. et al. | Minus | 26095902-26095502 |
| 337407 | Dunham, I. et al. | Minus | 31886652-31886567 |
| 337429 | Dunham, I. et al. | Minus | 32086238-32086079 |
| 337603 | Dunham, I. et al. | Minus | 1299296-1299194 |
| 338116 | Dunham, I. et al. | Minus | 10614071-10613814 |
| 338158 | Dunham, I. et al. | Minus | 11794465-11794343 |
| 338388 | Dunham, I. et al. | Minus | 18662403-18662305 |
| 338645 | Dunham, I. et al. | Minus | 24063839-24063775 |
| 338728 | Dunham, I. et al. | Minus | 25949039-25948927 |
| 339236 | Dunham, I. et al. | Minus | 32773355-32773202 |
| 339403 | Dunham, I. et al. | Minus | 34050728-34050625 |
| 325222 | 6525287 | Minus | 22332-22473 |
| 325251 | 6682448 | Minus | 411693-411751 |
| 325544 | 6682452 | Plus | 171228-171286 |
| 325753 | 6682474 | Plus | 398512-398621 |
| 329745 | 6065779 | Plus | 174774-175142 |
| 329732 | 6065783 | Plus | 161252-161322 |
| 329863 | 6691797 | Plus | 196801-196971 |
| 325889 | 5867087 | Plus | 223829-223891 |
| 325843 | 6552453 | Minus | 7126-7232 |
| 330020 | 6671887 | Plus | 172397-172491 |
| 326198 | 5867215 | Minus | 80295-80674 |
| 326230 | 5867230 | Minus | 301868-301972 |
| 326169 | 5867255 | Minus | 128321-128388 |
| 326077 | 6682495 | Minus | 312108-312168 |
| 326080 | 6682495 | Plus | 478644-478847 |
| 326759 | 6249610 | Plus | 97216-97311 |
| 326788 | 6682503 | Plus | 277132-277335 |
| 326946 | 6004446 | Minus | 116677-116967 |
| 327036 | 6531965 | Plus | 319951-320040 |
| 327220 | 5867525 | Minus | 65701-65781 |
| 327365 | 6552412 | Minus | 118133-118198 |
| 327414 | 5867750 | Plus | 102461-102586 |
| 327373 | 5867792 | Minus | 8186-8742 |
| 327568 | 5867811 | Minus | 46152-46287 |
| 330187 | 6706138 | Plus | 212923-213020 |
| 327747 | 5867947 | Plus | 115322-115498 |
| 327844 | 6249582 | Minus | 18895-18958 |
| 330232 | 6013526 | Plus | 113655-113830 |
| 328229 | 5868105 | Minus | 120936-121053 |
| 327876 | 5868140 | Plus | 103882-104034 |
| 328262 | 6381906 | Plus | 11867-12027 |
| 328688 | 5868262 | Plus | 626030-626094 |
| 328700 | 5868264 | Plus | 764089-764203 |
| 328695 | 5868264 | Plus | 318632-318695 |
| 328418 | 5868409 | Minus | 258811-258894 |
| 328455 | 5868431 | Plus | 385576-385633 |
| 328520 | 5868477 | Plus | 1942075-1942246 |
| 328548 | 5868487 | Plus | 72301-72397 |
| 328857 | 6381927 | Minus | 80557-81051 |
| 330305 | 4877982 | Minus | 52269-52365 |
| 330306 | 4877982 | Plus | 96161-96233 |
| 328937 | 5868500 | Minus | 1448241-1448333 |
| 328957 | 6456773 | Plus | 219195-219297 |
| 329073 | 5868596 | Plus | 37838-37956 |
| 329088 | 5868608 | Plus | 116738-116950 |
| 329350 | 6456785 | Plus | 98911-98969 |
| 329414 | 5868874 | Plus | 942555-942643 |
| 329440 | 5868885 | Plus | 21943-22063 |
| 329451 | 5868887 | Plus | 25974-26048 |

Table 6B shows the genomic positioning for those pkeys lacking unigeneID's and accession numbers in Table 6. The pkeys in Table 7 lacking unigeneID's are represented within Tables 1-6B For each predicted exon, we have listed the genomic sequence source used for prediction. Nucleotide locations of each predicted exon are also listed.
Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402:489-495
Strand: Indicates DNA strand from which exons were predicted
Nt_position: Indicates nucleotide positions of predicted exons

TABLE 7
Table 7 depicts Seq ID No, Unigene ID, Unigene Title, Pkey, and ExAccn for all of the sequences in Table 8.
Seq ID No links the nucleic acid and protein sequence information in Table 8 to Table 7.

| PKey | ExAccn | Unigene ID | Unigene Tiltle | SEQ ID NO |
|---|---|---|---|---|
| 101545 | BE246154 | Hs. 154210 | endothelial differentiation, sphingolipi | Seq ID 1 & 2 |
| 115819 | AA486620 | Hs. 41135 | endomucin-2 | Seq ID 3 & 4 |
| 424503 | NM_002205 | Hs. 149609 | integrin, alpha 5 (fibronectin receptor, | Seq ID 5 & 6 |
| 102917 | Al016712 | Hs. 287797 | integrin, beta 1 (fibronectin receptor, | Seq ID 7 & 8 |
| 102915 | X07820 | Hs. 2258 | matrix metalloproteinase 10 (stromelysin | Seq ID 9 & 10 |
| 105330 | AW338625 | Hs 22120 | ESTs | Seq ID 11 & 12 |
| 107385 | NM_005397 | Hs. 16426 | podocalyxin-like | Seq ID 13 & 14 |
| 102024 | AA301867 | Hs. 76224 | EGF-containing fibulin-like extracellula | Seq ID 15 & 16 |
| 102024 | AA301867 | Hs. 76224 | EGE-containing fibulin-like extracellula | Seq ID 17 & 18 |
| 134416 | X68264 | Hs. 211579 | melanoma cell adhesion molecule | Seq ID 19 & 20 |
| 103036 | M13509 | Hs. 83169 | matrix metalloproteinase 1 (interstitial | Seq ID 21 & 22 |
| 104865 | T79340 | Hs. 22575 | B-cell CLL/lymphoma 6, member B (zinc fi | Seq ID 23 & 24 |
| 106124 | H93366 | Hs. 7567 | Homo sapiens cDNA: FLJ21962 fis, clone H | Seq ID 25 & 26 |
| 109001 | Al056548 | Hs. 72116 | hypothetical protein FLJ20992 similar to | Seq ID 27 & 28 |
| 104764 | Al039243 | Hs. 278585 | ESTs | Seq ID 29 & 30 |
| 133200 | AB037715 | Hs. 183639 | hypothetical protein FLJ10210 | Seq ID 31 & 32 |
| 105263 | AW388633 | Hs. 6682 | solute carrier family 7, (cationic amino | Seq ID 33 & 34 |
| 102892 | BE440042 | Hs. 83326 | matrix metalloproteinase 3 (stromelysin | Seq ID 35 & 36 |
| 109456 | AW956580 | Hs. 42699 | ESTs | Seq ID 37 & 38 |
| 110906 | AA035211 | Hs. 17404 | ESTs | Seq ID 39 & 40 |
| 119073 | BE245360 | Hs. 279477 | ESTs | Seq ID 41 & 42 |
| 132050 | Al267615 | Hs. 38022 | ESTs | Seq ID 43 & 44 |
| 132490 | NM_001290 | Hs. 4980 | LIM domain binding 2 | Seq ID 45 & 46 |
| 102283 | AW161552 | Hs. 83381 | guanine nucleotide binding protein 11 | Seq ID 47 & 48 |
| 101714 | M68874 | Hs. 211587 | phospholipase A2, group IVA (cytosolic, | Seq ID 49 & 50 |
| 133975 | C18356 | Hs. 295944 | tissue factor pathway inhibitor 2 | Seq ID 51 & 52 |
| 106793 | H94997 | Hs. 16450 | ESTs | Seq ID 53 & 54 |
| 118511 | N75620 | Hs. 43157 | ESTs | Seq ID 54 & 55 |
| 101447 | M21305 | | gb: Human alpha satellite and satellite 3 | Seq ID 56 & 57 |
| 314941 | AA515902 | Hs. 130650 | ESTs | Seq ID 58 & 59 |
| 332466 | AB018259 | Hs. 118140 | KIAA0716 gene product | Seq ID 60 & 61 |
| 313513 | AW298600 | Hs. 141840 | ESTs, Weakly similar to S59501 interfero | Seq ID 62 & 63 |
| 313556 | AA628517 | Hs. 118502 | ESTs | Seq ID 64 & 65 |
| 313665 | AW751201 | Hs. 51233 | ESTs | Seq ID 66 & 67 |
| 314372 | AL040178 | Hs. 142003 | ESTs | Seq ID 68 & 69 |
| 429276 | AF056085 | Hs. 198612 | G protein-coupled receptor 51 | Seq ID 70 & 71 |
| 101345 | NM_005795 | Hs. 152175 | calcitonin receptor-like | Seq ID 72 & 73 |
| 418994 | AA296520 | Hs. 89546 | selectin E (endothelial adhesion molecul | Seq ID 74 & 75 |
| 103850 | AA187101 | Hs. 213194 | hypothetical protein MGC10895 | Seq ID 76 & 77 |
| 133260 | AA403045 | Hs. 6906 | Homo sapiens cDNA: FLJ23197 fis, clone R | Seq ID 78 & 79 |
| 101097 | BE245301 | Hs. 89414 | chemokine (C-X-C motif, receptor 4 (fus | Seq ID 80 & 81 |
| 104786 | AA027167 | Hs. 10031 | KIAA0955 protein | Seq ID 82 & 83 |
| 132173 | X89426 | Hs. 41716 | endothelial cell-specific molecule 1 | Seq ID 84 & 85 |
| 100420 | D86983 | Hs. 118893 | Melanoma associated gene | Seq ID 86 & 87 |
| 111018 | A1287912 | Hs. 3628 | mitogen-activated protein kinase kinase | Seq ID 88 & 89 |
| 108507 | A1554545 | Hs. 68301 | ESTs | Seq ID 90 & 91 |
| 104894 | AF065214 | Hs. 18858 | phospholipase A2, group IVC (cytosolic, | Seq ID 92 & 93 |
| 118511 | N75620 | Hs. 43157 | ESTs | Seq ID 94 & 95 |
| 125609 | AA868063 | Hs. 104576 | carbohydrate (keratan sulfate Gal-6) sul | Seq ID 96 & 97 |
| 101543 | M31166 | Hs. 2050 | pentaxin-related gene, rapidly induced b | Seq ID 98 & 99 |
| 102241 | NM_007351 | Hs. 268107 | multimerin | Seq ID 100 & 101 |
| 101560 | AW958272 | Hs. 347326 | intercellular adhesion molecule 2 | Seq ID 102 & 103 |
| 103280 | U84722 | Hs. 76206 | cadherin 5, type 2, VE-cadherin (vascula | Seq ID 104 & 105 |
| 105826 | AA478756 | Hs. 194477 | E3 ubiquitin ligase SMURF2 | Seq ID 106 & 107 |
| 102804 | NM_002318 | Hs. 83354 | lysyl oxidase-like 2 | Seq ID 108 & 109 |
| 131647 | AA359615 | Hs. 30089 | ESTs | Seq ID 110 & 111 |
| 103095 | NM_005424 | Hs. 78824 | tyrosine kinase with immunoglobulin and | Seq ID 112 & 113 |
| 103037 | BE018302 | Hs. 2894 | placental growth factor, vascular endoth | Seq ID 114 & 115 |
| 100405 | AW291587 | Hs. 82733 | nidogen 2 | Seq ID 116 & 117 |
| 102012 | BE259035 | Hs. 118400 | singed (Drosophila)-like (sea urchin fas | Seq ID 118 & 119 |
| 101261 | D30857 | Hs. 82353 | protein C receptor, endothelial (EPCR) | Seq ID 120 & 121 |
| 105729 | H46612 | Hs. 293815 | Homo sapiens HSPC285 mRNA, partial cds | Seq ID 122 & 123 |
| 107216 | D51069 | Hs. 211579 | melanoma cell adhesion molecule | Seq ID 124 & 125 |
| 131080 | NM_001955 | Hs. 2271 | endothelin 1 | Seq ID 126 & 127 |
| 131486 | F06972 | Hs. 27372 | BMX non-receptor tyrosine kinase | Seq ID 128 & 129 |
| 134299 | AW580939 | Hs. 97199 | complement component C1q receptor | Seq ID 130 & 131 |
| 134983 | D28235 | Hs. 196384 | prostaglandin-endoperoxide synthase 2 (p | Seq ID 132 & 133 |
| 115827 | AA428000 | Hs. 283072 | actin related protein 2/3 complex, subun | Seq ID 134 & 135 |
| 133614 | NM_003003 | Hs. 75232 | SEC14 (S. cerevisiae)-like 1 | Seq ID 136 & 137 |
| 116483 | A1346201 | Hs. 76118 | ubiquitin carboxyl-terminal esterase L1 | Seq ID 138 & 139 |
| 132546 | M24283 | Hs. 168383 | intercellular adhesion molecule 1 (CD54) | Seq ID 140 & 141 |
| 133678 | AW247252 | NA | nucleoside phosphorylase | Seq ID 142 & 143 |
| 130184 | H58306 | Hs. 15165 | retinoic acid induced 14 | Seq ID 144 & 145 |
| 134786 | T29618 | Hs. 89640 | TEK tyrosine kinase, endothelial (venous | Seq ID 146 & 147 |
| 129371 | X06828 | Hs. 110802 | von Willebrand factor | Seq ID 148 & 149 |
| 418506 | AA084248 | Hs. 85339 | G protein-coupled receptor 39 | Seq ID 150 & 151 |
| 322262 | AA632012 | Hs. 188746 | ESTs | Seq ID 152 & 153 |
| 312173 | Al821409 | Hs. 304471 | EST | Seq ID 154 & 155 |
| 319795 | AB037821 | Hs. 146858 | protocadherin 10 | Seq ID 156 & 157 |
| 313978 | Al870175 | Hs. 13957 | ESTs | Seq ID 158 & 159 |
| 306840 | Al077477 | Hs. 307912 | ESTs | Seq ID 160 & 161 |
| 310272 | AF216389 | Hs. 148932 | sema domain, transmembrane domain (TM), | Seq ID 162 & 163 |
| 310272 | AF216389 | Hs. 148932 | sema domain, transmembrane domain (TM), | Seq ID 164 & 165 |
| 315044 | BE547674 | Hs. 204169 | ESTs, Weakly similar to S65657 alpha-1C- | Seq ID 166 & 167 |
| 321325 | AB033100 | Hs. 300646 | KIAA1274 protein (similar to mouse palad | Seq ID 168 & 169 |
| 321325 | AB033100 | Hs. 300646 | KIAA1274 protein (similar to mouse palad | Seq ID 170 & 171 |
| 303251 | AF240635 | Hs. 115897 | protocadherin 12 | Seq ID 172 & 173 |
| 302378 | AL109712 | Hs. 296506 | Homo sapiens mRNA full length insert cDN | Seq ID 174 & 175 |
| 319267 | F11802 | Hs. 6818 | ESTs | Seq ID 176 & 177 |
| 310442 | AW072215 | Hs. 208470 | ESTs | Seq ID l78 & 179 |
| 300469 | BE3O1708 | Hs. 233955 | hypothetical protein FLJ20401 | Seq ID 180 & 181 |
| 331237 | W87874 | Hs. 25277 | Homo sapiens cDNA FLJ10717 fis; clone NT | Seq ID 182 & 183 |
| 330968 | R44557 | Hs. 23748 | ESTs | Seq ID 184 & 185 |
| 301822 | X17033 | Hs. 271986 | integrin, alpha 2 (CD49B, alpha 2 subuni | Seq ID 186 & 187 |
| 422573 | AW297985 | Hs. 295726 | integrin, alpha V (vitronectin receptor | Seq ID 188 & 189 |
| 133061 | All86431 | Hs. 296638 | prostate differentiation factor | Seq ID 190 & 191 |
| 135235 | AW298244 | Hs. 266195 | ESTs | Seq ID 192 & 193 |
| 101192 | BE247295 | Hs. 78452 | solute carrier family 20 (phosphate tran | Seq ID 194 & 195 |
| 113195 | H83265 | Hs. 8881 | ESTs, Weakly similar to S41044 chromosom | Seq ID 196 & 197 |
| 101741 | NM_003199 | Hs. 326198 | transcription factor 4 | Seq ID 198 & 199 |
| 321911 | AF026944 | Hs. 293797 | ESTs | Seq ID 200 & 201 |
| 320635 | N50617 | Hs. 80506 | small nuclear ribonucleoprotein polypept | Seq ID 202 & 203 |
| 326230 | | | NM_017643: Homo sapiens hypothetical prot | Seq ID 204 & 205 |
| 132968 | AF234532 | Hs. 61638 | myosin X | Seq ID 206 & 207 |
| 135073 | W55956 | Hs. 94030 | Homo sapiens mRNA; cDNA DKFZp586E1624 (f | Seq ID 208 & 209 |
| 108937 | AL050107 | Hs. 24341 | transcriptional co-activator with PDZ-bi | Seq ID 210 & 211 |
| 116430 | AK001531 | Hs. 66048 | hypothetical protein FLJ10669 | Seq ID 212 & 213 |
| 104877 | Al138635 | Hs. 22968 | Homo sapiens clone IMAGE: 451939, mRNA se | Seq ID 214 & 215 |
| 122697 | AA420683 | Hs. 98321 | hypothetical protein FLJ141O3 | Seq ID 216 & 217 |
| 112522 | R68857 | Hs. 265499 | ESTs | Seq ID 218 & 219 |
| 304782 | AA582081 | | gb:nn32h08.s1 NCI_CGAP_Gas1 Homo sapiens | Seq ID 220 & 221 |
| 312802 | AA644669 | Hs. 193042 | ESTs | Seq ID 222 & 223 |
| 302680 | AW192334 | Hs. 38218 | ESTs | Seq ID 224 & 225 |
| 326198 | | | Phase 2 & 3 Exons | Seq ID 226 & 227 |
| 331019 | NM_006033 | Hs. 65370 | lipase; endothelial | Seq ID 228 & 229 |

Pkey: Unique Eos probeset identifier number
ExAccn: Exemplar Accession number, Genbank accession number
Unigene ID: Unigene number
Unigene Title: Unigene gene title
Seq.ID No: Sequence Identification Number found in Table 8

TABLE 8

Seq ID NO: 1 DNA sequence
Nucleic Acid Accession #: NM_001400
Coding sequence: 244-2208 (underlined sequences correspond to start
and stop codons))

| | |
|---|---|
| 1 11 21 31 41 51 | |
| \| \| \| \| \| \| | |
| GTCGGGGGCA GCAGCAAGAT GCGAAGCGAG CCGTACAGAT CCCGGGCTCT CCGAACGCAA | 60 |
| CTTCGCCCTG CTTGAGCGAG GCTGCGGTTT CCGAGGCCCT CTCCAGCCAA GGAAAAGCTA | 120 |
| CACAAAAAGC CTGGATCACT CATCGAACCA CCCCTGAAGC CAGTGAAGGC TCTCTCGCCT | 180 |
| CGCCCTCTAG CGTTCGTCTG GAGTAGCGCC ACCCCGGCTT CCTGGGGACA CAGGGTTGGC | 240 |
| ACCATGGGGC CCACCAGCGT CCCGCTGGTC AAGGCCCACC GCAGCTCGGT CTCTGACTAC | 300 |
| GTCAACTATG ATATCATCGT CCGGCATTAC AACTACACGG GAAAGCTGAA TATCAGCGCG | 360 |
| GACAAGGAGA ACAGCATTAA ACTGACCTCG GTGGTGTTCA TTCTCATCTG CTGCTTTATC | 420 |
| ATCCTGGAGA ACATCTTTGT CTTGCTGACC ATTTGGAAAA CCAAGAAATT CCACCGACCC | 480 |
| ATGTACTATT TTATTGGCAA TCTGGCCCTC TCAGACCTGT TGGCAGGAGT AGCCTACACA | 540 |
| GCTAACCTGC TCTTGTCTGG GGCCACCACC TACAAGCTCA CTCCCGCCCA GTGGTTTCTG | 600 |
| CGGGAAGGGA GTATGTTTGT GGCCCTGTCA GCCTCCGTGT TCAGTCTCCT CGCCATCGCC | 660 |
| ATTGAGCGCT ATATCACAAT GCTGAAAATG AAACTCCACA ACGGGAGCAA TAACTTCCGC | 720 |
| CTCTTCCTGC TAATCAGCGC CTGCTGGGTC ATCTCCCTCA TCCTGGGTGG CCTGCCTATC | 780 |
| ATGGGCTGGA ACTGCATCAG TGCGCTGTCC AGCTGCTCCA CCGTGCTGCC GCTCTACCAC | 840 |
| AAGCACTATA TCCTCTTCTG CACCACGGTC TTCACTCTGC TTCTGCTCTC CATCGTCATT | 900 |
| CTGTACTGCA GAATCTACTC CTTGGTCAGG ACTCGGAGCC GCCGCCTGAC GTTCCGCAAG | 960 |
| AACATTTCCA AGGCCAGCCG CAGCTCTGAG AAGTCGCTGG CGCTGCTCAA GACCGTAATT | 1020 |
| ATCGTCCTGA GCGTCTTCAT CGCCTGCTGG GCACCGCTCT TCATCCTGCT CCTGCTGGAT | 1080 |
| GTGGGCTGCA AGGTGAAGAC CTGTGACATC CTCTTCAGAG CGGAGTACTT CCTGGTGTTA | 1140 |
| GCTGTGCTCA ACTCCGGCAC CAACCCCATC ATTTACACTC TGACCAACAA GGAGATGCGT | 1200 |
| CGGGCCTTCA TCCGGATCAT GTCCTGCTGC AAGTGCCCGA GCGGAGACTC TGCTGGCAAA | 1260 |
| TTCAAGCGAC CCATCATCGC CGGCATGGAA TTCAGCCGCA GCAAATCGGA CAATTCCTCC | 1320 |
| CACCCCCAGA AAGACGAAGG GGACAACCCA GAGACCATTA TGTCTTCTGG AAACGTCAAC | 1380 |
| TCTTCTTCCT AGAACTGGAA GCTGTCCACC CACCGGAAGC GCTCTTTACT TGGTCGCTGG | 1440 |
| CCACCCCAGT GTTTGGAAAA AAATCTCTGG GCTTCGACTG CTGCCAGGGA GGAGCTGCTG | 1500 |
| CAAGCCAGAG GGAGGAAGGG GGAGAATACG AACAGCCTGG TGGTGTCGGG TGTTGGTGGG | 1560 |
| TAGAGTTAGT TCCTGTGAAC AATGCACTGG GAAGGGTGGA GATCAGGTCC CGGCCTGGAA | 1620 |
| TATATATTCT ACCCCCCTGG AGCTTTGATT TTGCACTGAG CCAAAGGTCT AGCATTGTCA | 1680 |
| AGCTCCTAAA GGGTTCATTT GGCCCCTCCT CAAAGACTAA TGTCCCCATG TGAAAGCGTC | 1740 |
| TCTTTGTCTG GAGCTTTGAG GAGATGTTTT CCTTCACTTT AGTTTCAAAC CCAAGTGAGT | 1800 |
| GTGTGCACTT CTGCTTCTTT AGGGATGCCC TGTACATCCC ACACCCCACC CTCCCTTCCC | 1860 |
| TTCATACCCC TCCTCAACGT TCTTTTACTT TATACTTTAA CTACCTGAGA GTTATCAGAG | 1920 |
| CTGGGGTTGT GGAATGATCG ATCATCTATA GCAAATAGGC TATGTTGAGT ACGTAGGCTG | 1980 |
| TGGGAAGATG AAGATGGTTT GGAGGTGTAA AACAATGTCC TTCGCTGAGG CCAAAGTTTC | 2040 |
| CATGTAAGCG GGATCCGTTT TTTGGAATTT GGTTGAAGTC ACTTTGATTT CTTTAAAAAA | 2100 |
| CATCTTTTCA ATGAAATGTG TTACCATTTC ATATCCATTG AAGCCGAAAT CTGCATAAGG | 2160 |
| AAGCCCACTT TATCTAAATG ATATTAGCCA GGATCCTTGG TGTCCTAGGA GAAACAGACA | 2220 |
| AGCAAAACAA AGTGAAAACC GAATGGATTA ACTTTTGCAA ACCAAGGGAG ATTTCTTAGC | 2280 |
| AAATGAGTCT AACAAATATG ACATCCGTCT TTCCCACTTT TGTTGATGTT TATTTCAGAA | 2340 |
| TCTTGTGTGA TTCATTTCAA GCAACAACAT GTTGTATTTT GTTGTGTTAA AAGTACTTTT | 2400 |
| CTTGATTTTT GAATGTATTT GTTTCAGGAA GAAGTCATTT TATGGATTTT TCTAACCCGT | 2460 |
| GTTAACTTTT CTAGAATCCA CCCTCTTGTG CCCTTAAGCA TTACTTTAAC TGGTAGGGAA | 2520 |
| CGCCAGAACT TTTAAGTCCA GCTATTCATT AGATAGTAAT TGAAGATATG TATAAATATT | 2580 |
| ACAAAGAATA AAAATATATT ACTGTCTCTT TAGTATGGTT TTCAGTGCAA TTAAACCCAG | 2640 |
| AGATGTCTTG TTTTTTTAAA AAGAATAGTA TTTAATAGGT TTCTGACTTT TGTGGATCAT | 2700 |
| TTTGCACATA GCTTTATCAA CTTTTAAACA TTAATAAACT GATTTTTTTA AAG | |

Seq ID NO: 2 Protein sequence:
Protein Accession #: NP_001391

| | |
|---|---|
| 1 11 21 31 41 51 | |
| \| \| \| \| \| \| | |
| MGPTSVPLVK AHRSSVSDYV NYDIIVRHYN YTGKLNISAD KENSIKLTSV VFILICCFII | 60 |
| LENIFVLLTI WKTKKFHRPM YYFIGNLALS DLLAGVAYTA NLLLSGATTY KLTPAQWFLR | 120 |
| EGSMFVALSA SVFSLLAIAI ERYITMLKMK LHNGSNNFRL FLLISACWVI SLILGGLPIM | 180 |
| GWNCISALSS CSTVLPLYHK HYILFCTTVF TLLLLSIVIL YCRIYSLVRT RSRRLTFRKN | 240 |
| ISKASRSSEK SLALLKTVII VLSVFIACWA PLFILLLLDV GCKVKTCDIL FRAEYFLVLA | 300 |
| VLNSGTNPII YTLTNKEMRR AFIRIMSCCK CPSGDSAGKF KRPIIAGMEF SRSKSONSSH | 360 |
| PQKDEGDNPE TIMSSGNVNS SS | |

Seq ID NO: 3 Nucleotide sequence:
Nucleic Acid Accession #: NM_016242
Coding sequence: 79-864 (underlined sequences correspond to start and
stop codons))

| | |
|---|---|
| 1 11 21 31 41 51 | |
| \| \| \| \| \| \| | |
| AAGGCCCTGC CAGCTTGGGA GGGAATTGTC CCTGCCTGCT TCTGGAGAAA GAAGATATTG | 60 |
| ACACCATCTA CGGGCACCAT GGAACTGCTT CAAGTCACCA TTCTTTTTCT TCTGCCCAGT | 120 |
| ATTTGCACCA GTAACACCAC AGGTGTTTTA GAGGCAGCTA ATAATTCACT TGTTGTTACT | 180 |
| ACAACAAAAC CATCTATAAC AACACCAAAC ACAGAATCAT TACAGAAAAA TGTTGTCACA | 240 |
| CCAACAACTG GAACAACTCC TAAAGGAACA ATCACCAATG AATTACTTAA AATGTCTCTG | 300 |
| ATGTCAACAG CTACTTTTTT AACAAGTAAA GATGAAGGAT TGAAAGCCAC AACCACTGAT | 360 |
| GTCAGGAAGA ATGACTCCAT CATTTCAAAC GTAACAGTAA CAAGTGTTAC ACTTCCCAAT | 420 |
| GCTGTTTCAA CATTACAAAG TTCCAAACCC AAGACTGAAA CTCAGAGTTC AATTAAAACA | 480 |
| ACAGAAATAC CAGGTAGTGT TCTACAACCA GATGCATCAC CTTCTAAAAC TGGTACATTA | 540 |
| ACCTCAATAC CAGTTACAAT TCCAGAAAAC ACCTCACAGT CTCAAGTAAT AGACACTGAG | 600 |
| GGTGGAAAAA ATGCAAGCAC TTCAGCAACC AGCCGGTCTT ATTCCAGTAT TATTTTGCCG | 660 |
| GTGGTTATTG CTTTGATTGT AATAACACTT TCAGTATTTG TTCTGGTGGG TTTGTACCGA | 720 |
| ATGTGCTGGA AGGCAGATCC GGGCACACCA GAAAATGGAA ATGATCAACC TCAGTCTGAT | 780 |
| AAAGAGAGCG TGAAGCTTCT TACCGTTAAG ACAATTTCTC ATGAGTCTGG TGAGCACTCT | 840 |
| GCACAAGGAA AAACCAAGAA CTGACAGCTT GAGGAATTCT CTCCACACCT AGCCAATAAT | 900 |
| TACGCTTAAT CTTCAGCTTC TATGCACCAA GCGTGGAAAA GGAGAAAGTC CTGCAGAATC | 960 |
| AATCCCGACT TCCATACCTG CTGCTGG | |

Seq ID NO: 4 Protein sequence:
Protein Accession #: NP_057326

| | |
|---|---|
| 1 11 21 31 41 51 | |
| \| \| \| \| \| \| | |
| MELLQVTILF LLPSICSSNS TCVLEAANNS LVVTTTKPSI TTPNTESLQK NVVTPTTGTT | 60 |
| PKGTITNELL KMSLMSTATF LTSKDEGLKA TTTDVRKNDS IISNVTVTSV TLPNAVSTLQ | 120 |
| SSKPKTETQS SIKTTEIPGS VLQPDASPSK TGTLTSIPVT IPENTSQSQV IDTEGGKNAS | 180 |
| TSATSRSYSS IILPVVIALI VITLSVFVLV GLYRMCWKAD PGTPENGNDQ PQSDKESVKL | 240 |
| LTVKTISHES GEHSAQGKTK N | |

Seq ID NO: 5 Nucleotide sequence:
Nucleic Acid Accession #: NM_0022|
Coding sequence: 24 . . . 3173 (underlined sequences correspond to
start and stop codons)

| | |
|---|---|
| 1 11 21 31 41 51 | |
| \| \| \| \| \| \| | |
| CAGGACAGGG AAGAGCGGGC GCTATGGGGA GCCGCACGCC AGAGTCCCCT CTCCACGCCG | 60 |
| TGCAGCTGCG CTGGGGCCCC CGGCGCCGAC CCCCGCTCGT GCCGCTGCTG TTGCTGCTCG | 120 |
| TGCCGCCGCC ACCCAGGGTC GGGGGCTTCA ACTTAGACGC GGAGGCCCCA GCAGTACTCT | 180 |
| CGGGGCCCCC GGGCTCCTTC TTCGGATTCT CAGTGGAGTT TTACCGGCCG GGAACAGACG | 240 |
| GGGTCAGTGT GCTGGTGGGA GCACCCAAGG CTAATACCAG CCAGCCAGGA GTGCTGCAGG | 300 |
| GTGGTGCTGT CTACCTCTGT CCTTGGGGTG CCAGCCCCAC ACAGTGCACC CCCATTGAAT | 360 |
| TTGACAGCAA AGGCTCTCGG CTCCTGGAGT CCTCACTGTC CAGCTCAGAG GGAGAGGAGC | 420 |
| CTGTGGAGTA CAAGTCCTTG CAGTGGTTCG GGGCAACAGT TCGAGCCCAT GGCTCCTCCA | 480 |
| TCTTGGCATG CGCTCCACTG TACAGCTGGC GCACAGAGAA GGAGCCACTG AGCGACCCCG | 540 |
| TGGGCACCTG CTACCTCTCC ACAGATAACT TCACCCGAAT TCTGGAGTAT GCACCCTGCC | 600 |
| GCTCAGATTT CAGCTGGGCA GCAGGACAGG GTTACTGCCA AGGAGGCTTC AGTGCCGAGT | 660 |
| TCACCAAGAC TGGCCGTGTG GTTTTAGGTG GACCAGGAAG CTATTTCTGG CAAGGCCAGA | 720 |
| TCCTGTCTGC CACTCAGGAG CAGATTGCAG AATCTTATTA CCCCGAGTAC CTGATCAACC | 780 |
| TGGTTCAGGG GCAGCTGCAG ACTCGCCAGG CCAGTTCCAT CTATGATGAC AGCTACCTAG | 840 |
| GATACTCTGT GGCTGTTGGT GAATTCAGTG GTGATGACAC AGAAGACTTT GTTGCTGGTG | 900 |
| TGCCCAAAGG GAACCTCACT TACGGCTATG TCACCATCCT TAATGGCTCA GACATTCGAT | 960 |
| CCCTCTACAA CTTCTCAGGG GAACAGATGG CCTCCTACTT TGGCTATGCA GTGGCCGCCA | 1020 |
| CAGACGTCAA TGGGGACGGG CTGGATGACT TGCTGGTGGG GGCACCCCTG CTCATGGATC | 1080 |
| GGACCCCTGA CGGGCGGCCT CAGGAGGTGG GCAGGGTCTA CGTCTACCTG CAGCACCCAG | 1140 |
| CCGGCATAGA GCCCACGCCC ACCCTTACCC TCACTGGCCA TGATGAGTTT GGCCGATTTG | 1200 |
| GCAGCTCCTT GACCCCCCTG GGGGACCTGG ACCAGGATGG CTACAATGAT GTGGCCATCG | 1260 |
| GGGCTCCCTT TGGTGGGGAG ACCCAGCAGG GAGTAGTGTT TGTATTTCCT GGGGGCCCAG | 1320 |
| GAGGGCTGGG CTCTAAGCCT TCCCAGGTTC TGCAGCCCCT GTGGGCAGCC AGCCACACCC | 1380 |
| CAGACTTCTT TGGCTCTGCC CTTCGAGGAG GCCGAGACCT GGATGGCAAT GGATATCCTG | 1440 |
| ATCTGATTGT GGGGTCCTTT GGTGTGGACA AGGCTGTGGT ATACAGGGGC CGCCCCATCG | 1500 |
| TGTCCGCTAG TGCCTCCCTC ACCATCTTCC CCGCCATGTT CAACCCAGAG GAGCGGAGCT | 1560 |
| GCAGCTTAGA GGGGAACCCT GTGGCCTGCA TCAACCTTAG CTTCTGCCTC AATGCTTCTG | 1620 |
| GAAAACACGT TGCTGACTCC ATTGGTTTCA CAGTGGAACT TCAGCTGGAC TGGCAGAAGC | 1680 |
| AGAAGGGAGG GGTACGGCGG GCACTGTTCC TGGCCTCCAG GCAGGCAACC CTGACCCAGA | 1740 |
| CCCTGCTCAT CCAGAATGGG GCTCGAGAGG ATTGCAGAGA GATGAAGATC TACCTCAGGA | 1800 |
| ACGAGTCAGA ATTTCGAGAC AAACTCTCGC CGATTCACAT CGCTCTCAAC TTCTCCTTGG | 1860 |
| ACCCCCAAGC CCCAGTGGAC AGCCACGGCC TCAGGCCAGC CCTACATTAT CAGAGCAAGA | 1920 |
| GCCGGATAGA GGACAAGGCT CAGATCTTGC TGGACTGTGG AGAAGACAAC ATCTGTGTGC | 1980 |
| CTGACCTGCA GCTGGAAGTG TTTGGGGAGC AGAACCATGT GTACCTGGGT GACAAGAATG | 2040 |
| CCCTGAACCT CACTTTCCAT GCCCAGAATG TGGGTGAGGG TGGCGCCTAT GAGGCTGAGC | 2100 |
| TTCGGGTCAC CGCCCCTCCA GAGGCTGAGT ACTCAGGACT CGTCAGACAC CCAGGGAACT | 2160 |
| TCTCCAGCCT GAGCTGTGAC TACTTTGCCG TGAACCAGAG CCGCCTGCTG GTGTGTGACC | 2220 |
| TGGGCAACCC CATGAAGGCA GGAGCCAGTC TGTGGGGTGG CCTTCGGTTT ACAGTCCCTC | 2280 |
| ATCTCCGGGA CACTAAGAAA ACCATCCAGT TTGACTTCCA GATCCTCAGC AAGAATCTCA | 2340 |
| ACAACTCGCA AAGCGACGTG GTTTCCTTTC GGCTCTCCGT GGAGGCTCAG GCCCAGGTCA | 2400 |
| CCCTGAACGG TGTCTCCAAG CCTGAGGCAG TGCTATTCCC AGTAAGCGAC TGGCATCCCC | 2460 |
| GAGACCAGCC TCAGAAGGAG GAGGACCTGG GACCTCCTGT CCACCATGTC TATGAGCTCA | 2520 |
| TCAACCAAGG CCCCAGCTCC ATTAGCCAGG GTGTGCTGGA ACTCAGCTGT CCCCAGGCTC | 2580 |
| TGGAACGTCA GCAGCTCCTA TATGTGACCA GAGTTACGGG ACTCAACTGC ACCACCAATC | 2640 |
| ACCCCATTAA CCCAAAGGGC CTGGAGTTGC ATCCCGAGGG TTCCCTGCAC CACCAGCAAA | 2700 |
| AACGGGAAGC TCCAAGCCGC AGCTCTGCTT CCTCGGGACC TCAGATCCTG AAATGCCCGG | 2760 |
| AGGCTGAGTG TTTCAGGCTG CGCTGTCAGC TCGGGCCCCT GCACCAACAA GAGAGCCAAA | 2820 |
| GTCTGCAGTT GCATTTCCGA GTCTGGGCCA AGACTTTCTT GCAGCGGGAG CACCAGCCAT | 2880 |
| TTAGCCTGCA GTGTGAGGCT GTGTACAAAG CCCTGAAGAT GCCCTACCGA ATCCTGCCTC | 2940 |
| GGCAGCTGCC CCAAAAAGAG CGTCAGGTGG CCACAGCTGT GCAATGGACC AAGGCAGAAG | 3000 |
| GCAGCTATGG CGTCCCACTG TGGATCATCA TCCTAGCCAT CCTGTTTGGC CTCCTGCTCC | 3060 |
| TAGGTCTACT CATCTACATC CTCTACAAGC TTGGATTCTT CAAACGCTCC CTCCCATATG | 3120 |
| GCACCGCCAT GGAAAAAGCT CAGCTCAAGC CTCCAGCCAC CTCTGATGCC TGAGTCCTCC | 3180 |
| CAATTTCAGA CTCCCATTCC TGAAGAACCA GTCCCCCCAC CCTCATTCTA CTGAAAAGGA | 3240 |
| GGGGTCTGGG TACTTCTTGA AGGTGCTGAC GGCCAGGGAG AAGCTCCTCT CCCCAGCCCA | 3300 |
| GAGACATACT TGAAGGGCCA GAGCCAGGGG GGTGAGGAGC TGGGGATCCC TCCCCCCCAT | 3360 |
| GCACTGTGAA GGACCCTTGT TTACACATAC CCTCTTCATG GATGGGGGAA CTCAGATCCA | 3420 |
| GGGACAGAGG CCCAGCCTCC CTGAAGCCTT TGCATTTTGG AGAGTTTCCT GAAACAACTG | 3480 |
| GAAAGATAAC TAGGAAATCC ATTCACAGTT CTTTGGGCCA GACATGCCAC AAGGACTTCC | 3540 |
| TGTCCAGCTC CAACCTGCAA AGATCTGTCC TCAGCCTTGC CAGAGATCCA AAAGAAGCCC | 3600 |
| CCAGTAAGAA CCTGGAACTT GGGGAGTTAA GACCTGGCAG CTCTGGACAG CCCCACCCTG | 3660 |
| GTGGGCCAAC AAAGAACACT AACTATGCAT GGTGCCCCAG GACCAGCTCA GCACAGATGC | 3720 |
| CACAAGGATA GATGCTGGCC CAGGGCCAGA GCCCAGCTCC AAGGGGAATC AGAACTCAAA | 3780 |
| TGGGGCCAGA TCCAGCCTGG GGTCTGGAGT TGATCTGGAA CCCAGACTCA GACATTGGCA | 3840 |
| CCAATCCAGG CAGATCCAGG ACTATATTTG GGCCTGCTCC AGACCTGATC CTGGAGGCCC | 3900 |
| AGTTCACCCT GATTTAGGAG AAGCCAGGAA TTTCCCAGGA CCTGAAGGGG CCATGATGGC | 3960 |
| AACAGATCTG GAACCTCAGC CTGGCCAGAC ACAGGCCCTC CCTGTTCCCC AGAGAAAGGG | 4020 |
| GAGCCCACTG TCCTGGGCCT GCAGAATTTG GGTTCTGCCT GCCAGCTGCA CTGATGCTGC | 4080 |
| CCCTCATCTC TCTGCCCAAC CCTTCCCTCA CCTTGGCACC AGACACCCAG GACTTATTTA | 4140 |
| AACTCTGTTG CAAGTGCAAT AAATCTGACC CAGTGCCCCC ACTGACCAGA ACTAGAAAAA | 4200 |
| AAAA | |

Seq ID NO: 6 Protein sequence:
Protein Accession #: NP_002196.1

| | |
|---|---|
| 1 11 21 31 41 51 | |
| \| \| \| \| \| \| | |
| MGSRTPESPL HAVQLRWGPR RRPPLVPLLL LLVPPPPRVG GFNLDAEAPA VLSGPPGSFF | 60 |
| GFSVEFYRPG TDGVSVLVGA PKANTSQPGV LQGGAVYLCP WGASPTQCTP IEFDSKGSRL | 120 |
| LESSLSSSEG EEPVEYKSLQ WFGATVRAHG SSILACAPLY SWRTEKEPLS DPVGTCYLST | 180 |
| DNFTRILEYA PCRSDFSWAA GQGYCQGGFS AEFTKTGRVV LGGPGSYFWQ GQILSATQEQ | 240 |
| IAESYYPEYL INLVQGQLQT RQASSIYDDS YLGYSVAVGE FSGDDTEDFV AGVPKGNLTY | 300 |
| GYVTILNGSD IRSLYNFSGE QMASYFGYAV AATDVNGDGL DDLLVGAPLL MDRTPDGRPQ | 360 |
| EVGRVYVYLQ HPAGIEPTPT LTLTGHDEFG RFGSSLTPLG DLDQDGYNDV AIGAPFGGET | 420 |
| QQGVVFVFPG GPGGLGSKPS QVLQPLWAAS HTPDFFGSAL RGGRDLDGNG YPDLIVGSFG | 480 |
| VDKAVVYRGR PIVSASASLT IFPAMFNPEE RSCSLEGNPV ACINLSFCLN ASGKHVADSI | 540 |
| GFTVELQLDW QKQKGGVRRA LFLASRQATL TQTLLIQNGA REDCREMKIY LRNESEFRDK | 600 |
| LSPIHIALNF SLDPQAPVDS HGLRPALHYQ SKSRIEDKAQ ILLDCGEDNI CVPDLQLEVF | 660 |
| GEQNHVYLGD KNALNLTFHA QNVGEGGAYE AELRVTAPPE AEYSGLVRHP GNFSSLSCDY | 720 |
| FAVNQSRLLV CDLGNPMKAG ASLWGGLRFT VPHLRDTKKT IQFDFQILSK NLNNSQSDVV | 780 |
| SFRLSVEAQA QVTLNGVSKP EAVLFPVSDW HPRDQPQKEE DLGPAVHHVY ELINQGPSSI | 840 |
| SQGVLELSCP QALEGQQLLY VTRVTGLNCT TNHPINPKGL ELDPEGSLHH QQKREAPSRS | 900 |
| SASSGPQILK CPEAECFRLR CELGPLHQQE SQSLQLHFRV WAKTFLQREH QPFSLQCEAV | 960 |
| YKALKMPYRI LPRQLPQKER QVATAVQWTK AEGSYGVPLW IIILAILFGL LLLGLLIYIL | 1020 |
| YKLGFFKRSL PYGTAMEKAQ LKPPATSDA | |

Seq ID NO: 7 Nucleotide sequence:
Nucleic Acid Accession #: NM_002211
Coding sequence: 104 . . . 2500 (underlined sequences correspond to
start and stop codons)

| | |
|---|---|
| 1 11 21 31 41 51 | |
| \| \| \| \| \| \| | |
| GTCCGCCAAA ACCTGCGCGG ATAGGGAAGA ACAGCACCCC GGCGCCGATT GCCGTACCAA | 60 |
| ACAAGCCTAA CGTCCGCTGG GCCCCGGACG CCGCGCGGAA AAGATGAATT TACAACCAAT | 120 |
| TTTCTGGATT GGACTGATCA GTTCAGTTTG CTGTGTGTTT GCTCAAACAG ATGAAAATAC | 180 |
| ATGTTTAAAA GCAAATGCCA AATCATGTGG AGAATGTATA CAAGCAGGGC CAAATTGTGG | 240 |
| GTGGTGCACA AATTCAACAT TTTTACAGGA AGGAATGCCT ACTTCTGCAC GATGTGATGA | 300 |
| TTTAGAAGCC TTAAAAAAGA AGGGTTGCCC TCCAGATGAC ATAGAAAATC CCAGAGGCTC | 360 |
| CAAAGATATA AAGAAAAATA AAAATGTAAC CAACCGTAGC AAAGGAACAG CAGACAAGCT | 420 |
| CAAGCCAGAG GATATTACTC AGATCCAACC ACAGCAGTTG GTTTTGCGAT TAAGATCAGG | 480 |
| GGAGCCACAG ACATTTACAT TAAAATTCAA GAGAGCTGAA GACTATCCCA TTGACCTCTA | 540 |
| CTACCTTATG GACCTGTCTT ATTCAATGAA AGACGATTTG GAGAATGTAA AAAGTCTTGG | 600 |
| AACAGATCTG ATGAATGAAA TGAGGAGGAT TACTtCGGAC TTCAGAATTG GATTTGGCTC | 660 |
| ATTTGTGGAA AAGACTGTGA TGCCTTACAT TAGCACAACA CCAGCTAAGC TCAGGAACCC | 720 |
| TTGCACAAGT GAACAGAACT GCACCACCCC ATTTAGCTAC AAAAATGTGC TCAGTCTTAC | 780 |
| TAATAAAGGA GAAGTATTTA ATGAACTTGT TGGAAAACAG CGCATATCTG GAAATTTGGA | 840 |
| TTCTCCAGAA GGTGGTTTCG ATGCCATCAT GCAAGTTGCA GTTTGTGGAT CACTGATTGG | 900 |
| CTGGAGGAAT GTTACACGGC TGCTGGTGTT TTCCACAGAT GCCGGGTTTC ACTTTGCTGG | 960 |
| AGATGGGAAA CTTGGTGGCA TTGTTTTACC AAATGATGGA CAATGTCACC TGGAAAATAA | 1020 |
| TATGTACACA ATGAGCCATT ATTATGATTA TCCTTCTATT GCTCACCTTG TCCAGAAACT | 1080 |
| GAGTGAAAAT AATATTCAGA CAATTTTTGC AGTTACTGAA GAATTTCAGC CTGTTTACAA | 1140 |
| GGAGCTGAAA AACTTGATCC CTAAGTCAGC AGTAGGAACA TTATCTGCAA ATTCTAGCAA | 1200 |
| TGTAATTCAG TTGATCATTG ATGCATACAA TTCCCTTTCC TCAGAAGTCA TTTTGGAAAA | 1260 |
| CGGCAAATTG TCAGAAGGAG TAACAATAAG TTACAAATCT TACTGCAAGA ACGGGGTGAA | 1320 |
| TGGAACAGGG GAAAATGGAA GAAAATGTTC CAATATTTCC ATTGGAGATG AGGTTCAATT | 1380 |
| TGAAATTAGC ATAACTTCAA ATAAGTGTCC AAAAAAGGAT TCTGACAGCT TTAAAATTAG | 1440 |
| GCCTCTGGGC TTTACGGAGG AAGTAGAGGT TATTCTTCAG TACATCTGTG AATGTGAATG | 1500 |
| CCAAAGCGAA GGCATCCCTG AAAGTCCCAA GTGTCATGAA GGAAATGGGA CATTTGAGTG | 1560 |
| TGGCGCGTGC AGGTGCAATG AAGGGCGTGT TGGTAGACAT TGTGAATGCA GCACAGATGA | 1620 |
| AGTTAACAGT GAAGACATGG ATGCTTACTG CAGGAAAGAA AACAGTTCAG AAATCTGCAG | 1680 |
| TAACAATGGA GAGTGCGTCT GCGGACAGTG TGTTTGTAGG AAGAGGGATA ATACAAATGA | 1740 |
| AATTTATTCT GGCAAATTCT GCGAGTGTGA TAATTTCAAC TGTGATAGAT CCAATGGCTT | 1800 |
| AATTTGTGGA GGAAATGGTG TTTGCAAGTG TCGTGTGTGT GAGTGCAACC CCAACTACAC | 1860 |
| TGGCAGTGCA TGTGACTGTT CTTTGGATAC TAGTACTTGT GAAGCCAGCA ACGGACAGAT | 1920 |
| CTGCAATGGC CGGGGCATCT GCGAGTGTGG TGTCTGTAAG TGTACAGATC CGAAGTTTCA | 1980 |
| AGGGCAAACG TGTGAGATGT GTCAGACCTG CCTTGGTGTC TGTGCTGAGC ATAAAGAATG | 2040 |
| TGTTCAGTGC AGAGCCTTCA ATAAAGGAGA AAAGAAAGAC ACATGCACAC AGGAATGTTC | 2100 |
| CTATTTTAAC ATTACCAAGG TAGAAAGTCG GGACAAATTA CCCCAGCCGG TCCAACCTGA | 2160 |
| TCCTGTGTCC CATTGTAAGG ACAAGGATGT TGACGACTGT TGGTTCTATT TTACGTATTC | 2220 |
| AGTGAATGGG AACAACGAGG TCATGGTTCA TGTTGTGGAG AATCCAGAGT GTCCCACTGG | 2280 |
| TCCAGACATC ATTCCAATTG TAGCTGGTGT GGTTGCTGGA ATTGTTCTTA TTGGCCTTGC | 2340 |
| ATTACTGCTG ATATGGAAGC TTTTAATGAT AATTCATGAC AGAAGGGAGT TTGCTAAATT | 2400 |
| TGAAAAGGAG AAAATGAATG CCAAATGGGA CACGGGTGAA AATCCTATTT ATAAGAGTGC | 2460 |
| CGTAACAACT GTGGTCAATC CGAAGTATGA GGGAAAATGA GTACTGCCCG TGCAAATCCC | 2520 |
| ACAACACTGA ATGCAAAGTA GCAATTTCCA TAGTCACAGT TAGGTAGCTT TAGGGCAATA | 2580 |
| TTGCCATGGT TTTACTCATG TGCAGGTTTT GAAAATGTAC AATATGTATA ATTTTTAAAA | 2640 |
| TGTTTTATTA TTTTGAAAAT AATGTTGTAA TTCATGCCAG GGACTGACAA AAGACTTGAG | 2700 |
| ACAGGATGGT TATTCTTGTC AGCTAAGGTC ACATTGTGCC TTTTTGACCT TTTCTTCCTG | 2760 |
| GACTATTGAA ATCAAGCTTA TTGGATTAAG TGATATTTCT ATAGCGATTG AAAGGGCAAT | 2820 |
| AGTTAAAGTA ATGAGCATGA TGAGAGTTTC TGTTAATCAT GTATTAAAAC TGATTTTTAG | 2880 |
| CTTTACATAT GTCAGTTTGC AGTTATGCAG AATCCAAAGT AAATGTCCTG CTAGCTAGTT | 2940 |
| AAGGATTGTT TTAAATCTGT TATTTTGCTA TTTGCCTGTT AGACATGACT GATGACATAT | 3000 |
| CTGAAAGACA AGTATGTTGA GAGTTGCTGG TGTAAAATAC GTTTGAAATA GTTGATCTAC | 3060 |
| AAAGGCCATG GGAAAAATTC AGAGAGTTAG GAAGGAAAAA CCAATAGCTT TAAAACCTGT | 3120 |
| GTGCCATTTT AAGAGTTACT TAATGTTTGG TAACTTTTAT GCCTTCACTT TACAAATTCA | 3180 |
| AGCCTTAGAT AAAAGAACCG AGCAATTTTC TGCTAAAAAG TCCTTGATTT AGCACTATTT | 3240 |
| ACATACAGGC CATACTTTAC AAAGTATTTG CTGAATGGGG ACCTTTTGAG TTGAATTTAT | 3300 |
| TTTATTATTT TTATTTTGTT TAATGTCTGG TGCTTTCTAT CACCTCTTCT AATCTTTTAA | 3360 |
| TGTATTTGTT TGCAATTTTG GGGTAAGACT TTTTTATGAG TACTTTTTCT TTGAAGTTTT | 3420 |
| AGCGGTCAAT TTGCCTTTTT AATGAACATG TGAAGTTATA CTGTGGCTAT GCAACAGCTC | 3480 |
| TCACCTACGC GAGTCTTACT TTGAGTTAGT GCCATAACAG ACCACTGTAT GTTTACTTCT | 3540 |
| CACCATTTGA GTTGCCCATC TTGTTTCACA CTAGTCACAT TCTTGTTTTA AGTGCCTTTA | 3600 |
| GTTTTAACAG TTCA | |

Seq ID NO: 8 Protein sequence:
Protein Accession #: NP_002202

| | |
|---|---|
| 1 11 21 31 41 51 | |
| \| \| \| \| \| \| | |
| MNLQPIFWIG LISSVCCVFA QTDENRCLKA NAKSCGECIQ AGPNCGWCTN STFLQEGMPT | 60 |
| SARCDDLEAL KKKGCPPDDI ENPRGSKDIK KNKNVTNRSK GTAEKLKPED ITQIQPQQLV | 120 |
| LRLRSGEPQT FTLKFKRAED YPIDLYYLMD LSYSMKDDLE NVKSLGTDLM NEMRRITSDF | 180 |
| RIGFGSFVEK TVMPYISTTP AKLRNPCTSE QNCTSPFSYK NVLSLTNKGE VFNELVGKQR | 240 |
| ISGNLDSPEG GFDAIMQVAV CGSLIGWRNV TRLLVFSTDA GFHFAGDGKL GGIVLPNDGQ | 300 |
| CHLENNMYTM SHYYDYPSIA HLVQKLSENN IQTIFAVTEE FQPVYKELKN LIPKSAVGTL | 360 |
| SANSSNVIQL IIDAYNSLSS EVILENGKLS EGVTISYKSY CKNGVNGTGE NGRKCSNISI | 420 |
| GDEVQFEISI TSNKCPKKDS DSFKIRPLGF TEEVEVILQY ICECECQSEG IPESPKCHEG | 480 |
| NGTFECGACR CNEGRVGRHC ECSTDEVNSE DMDAYCRKEN SSEICSNNGE CVCGQCVCRK | 540 |
| RDNTNEIYSG KFCECDNFNC DRSNGLICGG NGVCKCRVCE CNPNYTGSAC DCSLDTSTCE | 600 |
| ASNGQICHGR GICECGVCKC TDPKFQGQTC EMCQTCLGVC AEHKECVQCR AFNKGEKKDT | 660 |
| CTQECSYFNI TKVESRDKLP QPVQPDPVSH CKEKDVDDCW FYFTYSVNGN NEVMVHVVEN | 720 |
| PECPTGPDII PIVAGVVAGI VLIGLALLLI WKLLMIIHDR REFAKFEKEK MNAKWDTGEN | 780 |
| PIYKSAVTTV VNPKYEGK | |

Seq ID NO: 9 Nucleotide sequence:
Nucleic Acid Accession #:NM_002425
Coding sequence: 23 . . . 1453 (underlined sequences correspond to
start and stop codons)

| | |
|---|---|
| 1 11 21 31 41 51 | |
| \| \| \| \| \| \| | |
| AAAGAAGGTA AGGGCAGTGA GAATGATGCA TCTTGCATTC CTTGTGCTGT TGTGTCTGCC | 60 |
| AGTCTGCTCT GCCTATCCTC TGAGTGGGGC AGCAAAAGAG GAGGACTCCA ACAAGGATCT | 120 |
| TGCCCAGCAA TACCTAGAAA ACTACTACAA CCTCGAAAAG GATGTGAAAC AGTTTAGAAG | 180 |
| AAAGGACAGT AATCTCATTG TTAAAAAAAT CCAAGGAATG CAGAAGTTCC TTGGGTTGGA | 240 |
| GGTGACAGGG AAGCTAGACA CTGACACTCT GGAGGTGATG CGCAAGCCCA GGTGTGGAGT | 300 |
| TCCTGACGTT GGTCACTTCA GCTCCTTTCC TGGCATGCCG AAGTGGAGGA AAACCCACCT | 360 |
| TACATACAGG ATTGTGAATT ATACACCAGA TTTGCCAAGA GATGCTGTTG ATTCTGCCAT | 420 |
| TGAGAAAGCT CTGAAAGTCT GGGAAGAGGT GACTCCACTC ACATTCTCCA GGCTGTATGA | 480 |
| AGGAGAGGCT GATATAATGA TCTCTTTCGC AGTTAAAGAA CATGGAGACT TTTACTCTTT | 540 |
| TGATGGCCCA GGACACAGTT TGGCTCATGC CTACCCACCT GGACCTGGGC TTTATGGAGA | 600 |
| TATTCACTTT GATGATGATG AAAAATGGAC AGAAGATGCA TCAGGCACCA ATTTATTCCT | 660 |
| CGTTGCTGCT CATGAACTTG GCCACTCCCT GGGGCTCTTT CACTCAGCCA ACACTGAAGC | 720 |
| TTTGATGTAC CCACTCTACA ACTCATTCAC AGAGCTCGCC CAGTTCCGCC TTTCGCAAGA | 780 |
| TGATGTGAAT GGCATTCAGT CTCTCTACGG ACCTCCCCCT GCCTCTACTG AGGAACCCCT | 840 |
| GGTGCCCACA AAATCTGTTC CTTCGGGATC TGAGATGCCA GCCAAGTGTG ATCCTGCTTT | 900 |
| GTCCTTCGAT GCCATCAGCA CTCTGAGGGG AGAATATCTG TTCTTTAAAG ACAGATATTT | 960 |
| TTGGCGAAGA TCCCACTGGA ACCCTGAACC TGAATTTCAT TTGATTTCTG CATTTTGGCC | 1020 |
| CTCTCTTCCA TCATATTTGG ATGCTGCATA TGAAGTTAAC AGCAGGGACA CCGTTTTTAT | 1080 |
| TTTTAAAGGA AATGAGTTCT GGGCCATCAG AGGAAATGAG GTACAAGCAG GTTATCCAAG | 1140 |
| AGGCATCCAT ACCCTGGGTT TTCCTCCAAC CATAAGGAAA ATTGATGCAG CTGTTTCTGA | 1200 |
| CAAGGAAAAG AAGAAAACAT ACTTCTTTGC AGCGGACAAA TACTGGAGAT TTGATGAAAA | 1260 |
| TAGCCAGTCC ATGGAGCAAG GCTTCCCTAG ACTAATAGCT GATGACTTTC CAGGAGTTGA | 1320 |
| GCCTAAGGTT GATGCTGTAT TACAGGCATT TGGATTTTTC TACTTCTTCA GTGGATCATC | 1380 |
| ACAGTTTGAG TTTGACCCCA ATGCCAGGAT GGTGACACAC ATATTAAAGA GTAACAGCTG | 1440 |
| GTTACATTGC TAGGCGAGAT AGGGGGAAGA CAGATATGGG TGTTTTTAAT AAATCTAATA | 1500 |
| ATTATTCATC TAATGTATTA TGAGCCAAAA TGGTTAATTT TTCCTGCATG TTCTGTGACT | 1560 |
| GAAGAAGATG AGCCTTGCAG ATATCTGCAT GTGTCATGAA GAATGTTTCT GGAATTCTTC | 1620 |
| ACTTGCTTTT GAATTGCACT GAACAGAATT AAGAAATACT CATGTGCAAT AGGTGAGAGA | 1680 |
| ATGTATTTTC ATAGATGTGT TATTACTTCC TCAATAAAAA GTTTTATTTT GGGCCTGTTC | 1740 |
| CTT | |

Seq ID NO: 10 Protein sequence:
Protein Accession #: NP_002416

| | |
|---|---|
| 1 11 21 31 41 51 | |
| \| \| \| \| \| \| | |
| MMHLAFLVLL CLPVCSAYPL SGAAKEEDSN KDLAQQYLEK YYNLEKDVKQ FRRKDSNLIV | 60 |
| KKIQGMQKFL GLEVTGKLDT DTLEVMRKPR CGVPDVGHFS SFPGMPKWRK THLTYRIVNY | 120 |
| TPDLPRDAVD SAIEKALKVW EEVTPLTFSR LYEGEADIMI SFAVKEHGDF YSFDGPGHSL | 180 |
| AHAYPPGPGL YGDIHFDDDE KWTEDASGTN LFLVAAHELG HSLGLFHSAN TEALMYPLYN | 240 |
| SFTELAQFRL SQDDVNGIQS LYGPPPASTE EPLVPTKSVP SGSEMPAKCD PALSFDAIST | 300 |
| LRGEYLFFKD RYFWRRSHWN PEPEFHLISA FWPSLPSYLD AAYEVNSRDT VFIFKGNEFW | 360 |
| AIRGNEVQAG YPRGIHTLGF PPTIRKIDAA VSDKEKKKTY FFAADKYWRF DENSQSMEQG | 420 |
| FPRLIADDFP GVEPKVDAVL QAFGFFYFFS GSSQFEFDPN ARMVTHILKS NSWLHC | |

Seq ID NO: 11 Nucleotide sequence:
Nucleic Acid Accession #: XM_058189
Coding sequence: 169 . . . 774 (underlined sequences correspond to
start and stop codons)

| | |
|---|---|
| 1 11 21 31 41 51 | |
| \| \| \| \| \| \| | |
| GAAGACCAGC TCAGCTCTTC AGTTGTTGAT CATTGTCTAT TGTTCTCCAA ACAGTAAACC | 60 |
| AGTATTTCAC ACTGAGATTG TCGGCTGCGG GTATATTCCA ATTCCCCGTC TCCTCATGAA | 120 |
| TATGAAGTGA AGGGCTCTGA CCCTGGAAGT GGTTCTAAGC ACGGCAAAAT GGGGTCTCGG | 180 |
| AAGTGTCGAG GCTGCCTAAG TTGTTTGCTG ATTCCGCTTG CACTTTGGAG TATAATCGTG | 240 |
| AACATATTAT TGTATTTCCC GAATGGGCAA ACTTCCTATG CATCCAGCAA TAAACTCACC | 300 |
| AACTACGTGT GGTATTTTGA AGGAATCTGT TTCTCAGGCA TCATGATGCT TATAGTAACA | 360 |
| ACAGTTCTTC TGGTACTGGA GAATAATAAC AACTATAAAT GTTGCCACAG TGAAAACTGC | 420 |
| AGCAAAAAAT ATGTGACACT GCTGTCAATT ATCTTTTCTT CCCTCGGAAT TGCTTTTTCT | 480 |
| GGATACTGCC TGGTCATCTC TGCCTTGGGT CTTGTCCAAG GGCCATATTG CCGCACCCTT | 540 |
| GATGGCTGGG AGTATGCTTT TGAAGGCACT GCTGGACGTT TCCTTACAGA TTCTAGCATA | 600 |
| TGGATTCAGT GCCTGGAACC TGCACATGTT GTGGAGTGGA ACATCATTTT ATTTTCCATT | 660 |
| CTCATAACCC TCAGTGGGCT TCAAGTGATC ATCTGCCTCA TCAGAGTAGT CATGCAACTA | 720 |
| TCCAAGATAC TGTGTGGAAG CTATTCAGTG ATCTTCCAGC CTGGAATCAT TTGAATAAGG | 780 |
| ACAAAATGTT TTCCATTATC AAGACATGGC CATCTATCTA AATATTATAT CAACTGTGTA | 840 |
| GACTTGAGGG CAATATTGAA ATGATGGTGC TTTCTGCATT TGGTGTTTAT TTGTAAAAAA | 900 |
| TTTGCAGTCC TCACTGCACA TGCAAGTATA CCACCCTTCC ATTTAGTATG TTTTTTAAGT | 960 |
| AATATGCATC AGAAACTTCA GAAATACTTC TGCCCTTTGA TCAAACAAAT CCATTTCCAA | 1020 |
| GAATCTGTAC TAGGGAAGTA AATAAGAATA TGAGAGAAAC CTTTATGCAA ATATGTATAT | 1080 |
| TGCAACATTA TTTAATATTC TGGAAAATTG GAAACACCCC AAAATTCTAA ACTCAGAGGA | 1140 |
| AGGATTAAGT AAAGAGTGGT ACATACTGTA AATGTTTTCT GATATTAAAA AAAAAATTAA | 1200 |
| ATAAAAAATA AAGAGTACTA CATGGTTGTA AAA | |

Seq ID NO: 12 Protein sequence:
Protein Accession #: XP_058189

| | |
|---|---|
| 1 11 21 31 41 51 | |
| \| \| \| \| \| \| | |
| MGSRKCGGCL SCLLIPLALW SIIVNILLYF PNGQTSYASS NKLTNYVWYF EGICFSGIMM | 60 |
| LIVTTVLLVL ENNNNYKCCQ SENCSKKYVT LLSIIFSSLG IAFSGYCLVI SALGLVQGPY | 120 |
| CRTLDGWEYA FEGTAORFLT DSSIWIQCLE PAHVVEWNII LFSILITLSG LQVIICLIRV | 180 |
| VMQLSKILCG SYSVIFQPGI I | |

Seq ID NO: 13 Nucleotide sequence:
Nucleic Acid Accession #: NM_005397
Coding sequence: 251 . . . 1837 (underlined sequences correspond to
start and stop codons)

| | |
|---|---|
| 1 11 21 31 41 51 | |
| \| \| \| \| \| \| | |
| AAACGCCGCC CAGGACGCAG CCGCCGCCGC CGCCGCTCCT CTGCCACTGG CTCTGCGCCC | 60 |
| CAGCCCGGCT CTGCTGCAGC GGCAGGGAGG AAGAGCCGCC GCAGCGCGAC TCGGGAGCCC | 120 |
| CGGGCCACAG CCTGGCCTCC GGAGCCACCC ACAGGCCTCC CCGGGCCGCG CCCACGCTCC | 180 |
| TACCGCCCGG ACGCGCGGAT CCTCCGCCGG CACCGCAGCC ACCTGCTCCC GGCCCAGAGG | 240 |
| CGACGACACG ATGCGCTGCG CGCTGGCGCT CTCGGCGCTG CTGCTACTGT TGTCAACGCC | 300 |
| GCCGCTGCTG CCGTCGTCGC CGTCGCCGTC GCCGTCGCCG TCGCCCTCCC AGAATGCAAC | 360 |
| CCAGACTACT ACGGACTCAT CTAACAAAAC AGCACCGACT CCAGCATCCA GTGTCACCAT | 420 |
| CATGGCTACA GATACAGCCC AGCAGAGCAC AGTCCCCACT TCCAAGGCCA ACGAAATCTT | 480 |
| GGCCTCGGTC AAGGCGACCA CCCTTGGTGT ATCCAGTGAC TCACCGGGGA CTACAACCCT | 540 |
| GGCTCAGCAA GTCTCAGGCC CAGTCAACAC TACCGTGGCT AGAGGAGGCG GCTCAGGCAA | 600 |
| CCCTACTACC ACCATCGAGA GCCCCAAGAG CACAAAAAGT GCAGACACCA CTACAGTTGC | 660 |
| AACCTCCACA GCCACAGCTA AACCTAACAC CACAAGCAGC CAGAATGGAG CAGAAGATAC | 720 |
| AACAAACTCT GGGGGGAAAA GCAGCCACAG TGTGACCACA GACCTCACAT CCACTAAGGC | 780 |
| AGAACATCTG ACGACCCCTC ACCCTACAAG TCCACTTAGC CCCCGACAAC CCACTTTGAC | 840 |
| GCATCCTGTG GCCACCCCAA CAAGCTCGGG ACATGACCAT CTTATGAAAA TTTCAAGCAG | 900 |
| TTCAAGCACT GTGGCTATCC CTGGCTACAC CTTCACAAGC CCGGGGATGA CCACCACCCT | 960 |
| ACCGTCATCG GTTATCTCGC AAAGAACTCA ACAGACCTCC AGTCAGATGC CAGCCAGCTC | 1020 |
| TACGGCCCCT TCCTCCCAGG AGACAGTGCA GCCCACGAGC CCGGCAACGG CATTGAGAAC | 1080 |
| ACCTACCCTC CCAGAGACCA TGAGCTCCAG CCCCACAGCA GCATCAACTA CCCACCGATA | 1140 |
| CCCCAAAACA CCTTCTCCCA CTGTGGCTCA TGAGAGTAAC TGGGCAAAGT GTGAGGATCT | 1200 |
| TGAGACACAG ACACAGAGTG AGAAGCAGCT CGTCCTGAAC CTCACAGGAA ACACCCTCTG | 1260 |
| TGCAGGGGGC GCTTCGGATG AGAAATTGAT CTCACTGATA TGCCGAGCAG TCAAAGCCAC | 1320 |
| CTTCAACCCG GCCCAAGATA AGTGCGGCAT ACGGCTGGCA TCTGTTCCAG GAAGTCAGAC | 1380 |
| CGTGGTCGTC AAAGAAATCA CTATTCACAC TAAGCTCCCT GCCAAGGATG TGTACGAGCG | 1440 |
| GCTGAAGGAC AAATGGGATG AACTAAAGGA GGCAGGGGTC AGTGACATGA AGCTAGGGGA | 1500 |
| CCAGGGGCCA CCGGAGGAGG CCGAGGACCG CTTCAGCATG CCCCTCATCA TCACCATCGT | 1560 |
| CTGCATGGCG TCATTCCTGC TCCTCGTGGC GGCCCTCTAT GGCTGCTGCC ACCAGCGCCT | 1620 |
| CTCCCAGAGG AAGGACCAGC AGCGGCTAAC AGAGGAGCTG CAGACAGTGG AGAATGGTTA | 1680 |
| CCATGACAAC CCAACACTGG AAGTGATGGA GACCTCTTCT GAGATGCAGG AGAAGAAGGT | 1740 |
| GGTCAGCCTC AACGGGGAGC TGGGGGACAG CTGGATCGTC CCTCTGGACA ACCTGACCAA | 1800 |
| GGACGACCTG GATGAGGAGG AAGACACACA CCTCTAGTCC GGTCTGCCGG TGGCCTCCAG | 1860 |
| CAGCACCACA GAGCTCCAGA CCAACCACCC CAAGTGCCGT TTGGATGGGG AAGGGAAAGA | 1920 |
| CTGGGGAGGG AGAGTGAACT CCGAGGGGTG TCCCCTCCCA ATCCCCCCAG GGCCTTAATT | 1980 |
| TTTCCCTTTT CAACCTGAAC AAATCACATT CTGTCCAGAT TCCTCTTGTA AAATAACCCA | 2040 |
| CTAGTGCCTG AGCTCAGTGC TGCTGGATGA TGAGGGAGAT CAAGAAAAAG CCACGTAAGG | 2100 |
| GACTTTATAG ATGAACTAGT GGAATCCCTT CATTCTGCAG TGAGATTGCC GAGACCTGAA | 2160 |
| GAGGGTAAGT GACTTGCCCA AGGTCAGAGC CACTTGGTGA CAGAGCCAGG ATGAGAACAA | 2220 |
| AGATTCCATT TGCACCATGC CACACTGCTG TGTTCACATG TGCCTTCCGT CCAGAGCAGT | 2280 |
| CCCGGGCAGG GGTGAAACTC CAGCAGGTGG CTGGGCTGGA AAGGAGGGCA GGGCTACATC | 2340 |
| CTGGCTCGGT GGGATCTGAC GACCTGAAAG TCCAGCTCCC AAGTTTTCCT TCTCCTACCC | 2400 |
| CAGCCTCGTG TACCCATCTT CCCACCCTCT ATGTTCTTAC CCCTCCCTAC ACTCAGTGTT | 2460 |
| TGTTCCCACT TACTCTGTCC TGGGGCCTCT GGGATTAGCA CAGGTTATTC ATAACCTTGA | 2520 |
| ACCCCTTGTT CTGGATTCGG ATTTTCTCAC ATTTGCTTCG TCAGATGGGG GCTTAACCCA | 2580 |
| CACAGGTCTC CGTGCGTGAA CCAGGTCTGC TTAGGGGACC TGCGTGCAGG TGAGGAGAGA | 2640 |
| AGGGGACACT CGAGTCCAGG CTGGTATCTC AGGGCAGCTG ATGAGGGGTC AGCAGCAACA | 2700 |
| CTGGCCCATT GCCCCTGGCA CTCCTTGCAG AGGCCACCCA CGATCTTCTT TGGGCTTCCA | 2760 |
| TTTCCACCAG GGACTAAAAT CTGCTGTAGC TAGTGAGAGC AGCGTGTTCC TTTTGTTGTT | 2820 |
| CACTGCTCAG CTGATGGGAG TGATTCCCTG AGACCCAGTA TGAAAGAGCA GTGGCTGCAG | 2880 |
| GAGAGGCCTT CCCGGGGCCC CCCATCAGCG ATGTGTCTTC AGAGACAATC CATTAAAGCA | 2940 |
| GCCAGGAAGG ACAGGCTTTC CCCTGTATAT CATAGGAAAC TCAGGGACAT TTCAAGTTGC | 3000 |
| TGAGAGTTTT GTTATAGTTG TTTTCTAACC CAGCCCTCCA CTGCCAAAGG CCAAAAGCTC | 3060 |
| AGACAGTTGG CAGACGTCCA GTTAGCTCAT CTCACTCACT CTGATTCTCC TGTGCCACAG | 3120 |
| GAAAAGAGGG CCTGGAAAGC GCAGTGCATG CTGGGTGCAT GAAGGGCAGC CTGGGGGACA | 3180 |
| GACTGTTGTG GGAACGTCCC ACTGTCCTGG CCTGGAGCTA GGCCTTGCTG TTCCTCTTCT | 3240 |
| CTGTGAGCCT AGTGGGGCTG CTGCGGTTCT CTTGCAGTTT CTGGTGGCAT CTCAGGGGAA | 3300 |
| CACAAAAGCT ATGTCTATTC CCCAATATAG GACTTTTATG GGCTCGGCAG TTAGCTGCCA | 3360 |
| TGTAGAAGGC TCCTAAGCAG TGGGCATGGT GAGGTTTCAT CTGATTGAGA AGGGGGAATC | 3420 |
| CTGTGTGGAA TCTTGAACTT TCGCCATGGT CTCCATCGTT CTGGGCGTAA ATTCCCTGGG | 3480 |
| ATCAAGTAGG AAAATGGGCA GAACTGCTTA GGGGAATGAA ATTGCCATTT TTCGGGTGAA | 3540 |
| ACGCCACACC TCCAGGGTCT TAAGAGTCAG GCTCCGGCTG TAGTAGCTCT GATGAAATAG | 3600 |
| GCTATCCACT CGGGATGGCT TACTTTTTAA AAGGGTAGGG GGAGGGGCTG GGGAAGATCT | 3660 |
| GTCCTGCACC ATCTGCCTAA TTCCTTCCTC ACAGTCTGTA GCCATCTGAT ATCCTAGGGG | 3720 |
| GAAAAGGAAG GCCAGGGGTT CACATAGGGC CCCAGCGAGT TTCCCAGGAG TTAGAGGGAT | 3780 |
| GCGAGGCTAA CAAGTTCCAA AAACATCTGC CCCGATGCTC TAGTGTTTGG AGGTGGGCAG | 3840 |
| GATGGAGAAC AGTGCCTGTT TGGGGGAAAA CAGGAAATCT TGTTAGGCTT GAGTGAGGTG | 3900 |
| TTTGCTTCCT TCTTGCCCAG CGCTGGGTTC TCTCCACCCA GTAGGTTTTC TGTTGTGGTC | 3960 |
| CCGTGGGAGA GGCCAGACTG GATTATTCCT CCTTTGCTGA TCCTGGGTCA CACTTCACCA | 4020 |
| GCCAGGGCTT TTGACGGAGA CAGCAAATAG GCCTCTGCAA ATCAATCAAA GGCTGCAACC | 4080 |
| CTATGGCCTC TTGGAGACAG ATGATGACTG GCAAGGACTA GAGAGCAGGA GTGCCTGGCC | 4140 |
| AGGTCGGTCC TGACTCTCCT GACTCTCCAT CGCTCTGTCC AAGGAGAACC CGGAGAGGCT | 4200 |
| CTGGGCTGAT TCAGAGGTTA CTGCTTTATA TTCGTCCAAA CTGTGTTAGT CTAGGCTTAG | 4260 |
| GACAGCTTCA GAATCTGACA CCTTGCCTTG CTCTTGCCAC CAGGACACCT ATGTCAACAG | 4320 |
| GCCAAACAGC CATGCATCTA TAAAGGTCAT CATCTTCTGC CACCTTTACT GGGTTCTAAA | 4380 |
| TGCTCTCTGA TAATTCAGAG AGCATTGGGT CTGGGAAGAG GTAAGAGGAA CACTAGAAGC | 4440 |
| TCAGCATGAC TTAAACAGGT TGTAGCAAAG ACAGTTTATC ATCAACTCTT TCAGTGGTAA | 4500 |
| ACTGTGGTTT CCCCAAGCTG CACAGGAGGC CAGAAACCAC AAGTATCATG ACTAGGAAGC | 4560 |
| CTACTGTCAT GAGAGTGGGG AGACAGGCAG CAAAGCTTAT GAAGGAGGTA CAGAATATTC | 4620 |
| TTTGCGTTGT AAGACAGAAT ACGGGTTTAA TCTAGTCTAG GCRCCAGATT TTTTTCCCGC | 4680 |
| TTGATAAGGA AAGCTAGCAG AAAGTTTATT TAAACCACTT CTTGAGCTTT ATCTTTTTTG | 4740 |
| ACAATATACT GGAGAAACTT TGAAGAACAA GTTCAAACTG ATACATATAC ACATATTTTT | 4800 |
| TTGATAATGT AAATACAGTG ACCATGTTAA CCTACCCTGC ACTGCTTTAA GTGAACATAC | 4860 |
| TTTGAAAAAG CATTATGTTA GCTGAGTGAT GGCCAAGTTT TTTCTCTGGA CAGGAATGTA | 4920 |
| AATGTCTTAC TGGAAATGAC AAGTTTTTGC TTGATTTTTT TTTTTAAACA AAAAATGAAA | 4980 |
| TATAACAAGA CAAACTTATG ATAAAGTATT TGTCTTGTAG ATCAGGTGTT TTGTTTTGTT | 5040 |
| TTTTTAATTT TAAAATGCAA CCCTGCCCCC TCCCCAGCAA AGTCACAGCT CCATTTCAGT | 5100 |
| AAAGGTTGGA GTCAATATGC TCTGGTTGGC AGGCAACCCT GTAGTCATGG AGAAAGGTAT | 5160 |
| TTCAAGATCT AGTCCAATCT TTTTCTAGAG AAAAAGATAA TCTGAAGCTC ACAAAGATGA | 5220 |
| AGTGACTTCC TCAAAATCAC ATGGTTCAGG ACAGAAACAA GATTAAAACC TGGATCCACA | 5280 |
| GACTGTGCGC CTCAGAAGGA ATAATCGGTA AATTAAGAAT TGCTACTCGA AGGTGCCAGA | 5340 |
| ATGACACAAA GGACAGAATT CCTTTCCCAG TTGTTACCCT AGCAAGGCTA GGGAGGGCAT | 5400 |
| GAACACAAAC ATAAGAACTG GTCTTCTCAC ACTTTCTCTG AATCATTTAG GTTTAAGATG | 5460 |
| TAAGTGAACA ATTCTTTCTT TCTGCCAAGA AACAAAGTTT TGGATGAGCT TTTATATATG | 5520 |
| GAACTTACTC CAACAGGACT GAGGGACCAA GGAAACATGA TGGGGGAGGC AAGAGAGCGC | 5580 |
| AAAGAGTAAA ACTGTAGCAT AGCTTTTGTC ACGGTCACTA GCTGATCCCT CAGGTCTGCT | 5640 |
| GCAAACACAG CATGGAGGAC ACAGATGACT CTTTGGTGTT GGTCTTTTTG TCTGCAGTGA | 5700 |
| ATGTTCAACA GTTTGCCCAG GAACTGGGGG ATCATATATG TCTTAGTGGA CAGGGGTCTG | 5760 |
| AAGTACACTG GAATTTACTG AGAAACTTGT TTGTAAAAAC TATAGTTAAT AATTATTGCA | 5820 |
| TTTTCTTACA AAAATATATT TTGGAAAATT GTATACTGTC AATTAAAGT | |

Seq ID NO: 14 Protein sequence:
Protein Accession #: NP_005388

| | |
|---|---|
| 1 11 21 31 41 51 | |
| \| \| \| \| \| \| | |
| MRCALALSAL LLLLSTPPLL PSSPSPSPSP SPSQNATQTT TDSSNKTAPT PASSVTIMAT | 60 |
| DTAQQSTVPT SKANEILASV KATTLGVSSD SPGTTTLAQQ VSGPVNTTVA RGGGSGNPTT | 120 |
| TIESPKSTKS ADTTTVATST ATAKPNTTSS QNGAEDTTNS GGKSSHSVTT DLTSTKAEHL | 180 |
| TTPHPTSPLS PRQPTLTHPV ATPTSSGHDH LMKISSSSST VAIPGYTFTS PGMTTTLPSS | 240 |
| VISQRTQQTS SQMPASSTAP SSQETVQPTS PATALRTPTL PETMSSSPTA ASTTHRYPKT | 300 |
| PSPTVAHESN WAKCEDLETQ TQSEKQLVLN LTGNTLCAGG ASDEKLISLI CRAVKATFNP | 360 |
| AQDKCGIRLA SVPGSQTVVV KEITIHTKLP AKDVYERLKD KWDELKEAGV SDMKLGDQGP | 420 |
| PEEAEDRFSM PLIITIVCMA SFLLLVAALY GCCHQRLSQR KDQQRLTEEL QTVENGYHDN | 480 |
| PTLEVMETSS EMQEKKVVSL NGELGDSWIV PLDNLTKDDL DEEEDTHL | |

Seq ID NO: 15 Nucleotide sequence:
Nucleic Acid Accession #: NM_004105
Coding sequence: 150 . . . 1631 (underlined sequences correspond to
start and stop codons)

| | |
|---|---|
| 1 11 21 31 41 51 | |
| \| \| \| \| \| \| | |
| CTAGTATTCT ACTAGAACTG GAAGATTGCT CTCCGAGTTT TTTTTTTGTT ATTTTGTTAA | 60 |
| AAAATAAAAA GCTTGAGCAG CAATTCATAT TACTGTCACA GGTATTTTTG CTGTGCTGTG | 120 |
| CAAGGTAACT CTGCTAGCTA AGATTCACAA TGTPTGAAAGC CCTTTTCCTA ACTATGCTGA | 180 |
| CTCTGGCGCT GGTCAAGTCA CAGGACACCG AAGAAACCAT CACGTACACG CAATGCACTG | 240 |
| ACGGATATGA GTGGGATCCT GTGAGACAGC AATGCAAAGA TATTGATGAA TGTGACATTG | 300 |
| TCCCAGACGC TTGTAAAGGT GGAATGAAGT GTGTCAACCA CTATGGAGGA TACCTCTGCC | 360 |
| TTCCGAAAAC AGCCCAGATT ATTGTCAATA ATGAACAGCC TCAGCAGGAA ACACAACCAG | 420 |
| CAGAAGGAAC CTCAGGGGCA ACCACCGGGG TTGTAGCTGC CAGCAGCATG GCAACCAGTG | 480 |
| GAGTGTTGCC CGGGGGTGGT TTTGTGGCCA GTGCTGCTGC AGTCGCAGGC CCTGAAATGC | 540 |
| AGACTGGCCG AAATAACTTT GTCATCCGGC GGAACCCAGC TGACCCTCAG CGCATTCCCT | 600 |
| CCAACCCTTC CCACCGTATC CAGTGTGCAG CAGGCTACGA GCAAAGTGAA CACAACGTGT | 660 |
| GCCAAGACAT AGACGAGTGC ACTGCAGGGA CGCACAACTG TAGAGGAGAC CAAGTGTGCA | 720 |
| TCAATTTACG GGGATCCTTT GCATGTCAGT GCCCTCCTGG ATATCAGAAG CGAGGGGAGC | 780 |
| AGTGCGTAGA CATAGATGAA TGTACCATCC CTCCATATTG CCACCAAAGA TGCGTGAATA | 840 |
| CACCAGGCTG ATTTTATTGC CAGTGCAGTC CTGGGTTTCA ATTGGCAGCA AACAACTATA | 900 |
| CCTGCGTAGA TATAAATGAA TGTGATGCCA GCAATCAATG TGCTCAGCAG TGCTACAACA | 960 |
| TTCTTGGTTC ATTCATCTGT CAGTGCAATC AAGGATATGA GCTAAGCAGT GACAGGCTCA | 1020 |
| ACTGTGAAGA CATTGATGAA TGCAGAACCT CAAGCTACCT GTGTCAATAT CAATGTGTCA | 1080 |
| ATGAACCTGG GAAATTCTCA TGTATGTGCC CCCAGGGATA CCAAGTGGTG AGAAGTAGAA | 1140 |
| CATGTCAAGA TATAAATGAG TGTGAGACCA CAAATGAATG CCGGGAGGAT GAAATGTGTT | 1200 |
| GGAATTATCA TGGCGGCTTC CGTTGTTATC CACGAAATCC TTGTCAAGAT CCCTACATTC | 1260 |
| TAACACCAGA GAACCGATGT GTTTGCCCAG TCTCAAATGC CATGTGCCGA GAACTGCCCC | 1320 |
| AGTCAATAGT CTACAAATAC ATGAGCATCC GATCTGATAG GTCTGTGCCA TCAGACATCT | 1380 |
| TCCAGATACA GGCCACAACT ATTTATGCCA ACACCATCAA TACTTTTCGG ATTAAATCTG | 1440 |
| GAAATGAAAA TGGAGAGTTC TACCTACGAC AAACAAGTCC TGTAAGTGCA ATGCTTGTGC | 1500 |
| TCGTGAAGTC ATTATCAGGA CCAAGAGAAC ATATCGTGGA CCTGGAGATG CTGACAGTCA | 1560 |
| GCAGTATAGG GACCTTCCGC ACAAGCTCTG TGTTAAGATT GACAATAATA GTGGGGCCAT | 1620 |
| TTTCATTTTA GTCTTTTCTA AGAGTCAACC ACAGGCATTT AAGTCAGCCA AAGAATATTG | 1680 |
| TTACCTTAAA GCACTATTTT ATTTATAGAT ATATCTAGTG CATCTACATC TCTATACTGT | 1740 |
| ACACTCACCC ATAACAAACA ATTACACCAT GGTATAAAGT GGGCATTTAA TATGTAAAGA | 1800 |
| TTCAAAGTTT GTCTTTATTA CTATATGTAA ATTAGACATT AATCCACTAA ACTGGTCTTC | 1860 |
| TTCAAGAGAG CTAAGTATAC ACTATCTGGT GAAACTTGGA TTCTTTCCTA TAAAAGTGGG | 1920 |
| ACCAAGCAAT GATGATCTTC TGTGGTGCTT AAGGAAACTT ACTAGAGCTC CACTAACAGT | 1980 |
| CTCATAAGGA GGCAGCCATC ATAACCATTG AATAGCATGC AAGGGTAAGA ATGAGTTTTT | 2040 |
| AACTGCTTTG TAAGAAAATG GAAAAGGTCA ATAAAGATAT ATTTCTTTAG AAAATGGGGA | 2100 |
| TCTGCCATAT TTGTGTTGGT TTTTATTTTC ATATCCAGCC TAAAGGTGGT TGTTTATTAT | 2160 |
| ATAGTAATAA ATCATTGCTG TACAACATGC TGGTTTCTGT AGGGTATTTT TAATTTTGTC | 2220 |
| AGAAATTTTA GATTGTGAAT ATTTTGTAAA AAACAGTAAG CAAAATTTTC CAGAATTCCC | 2280 |
| AAAATGAACC AGATACCCCC TAGAAAATTA TACTATTGAG AAATCTATGG GGAGGATATG | 2340 |
| AGAAAATAAA TTCCTTCTAA ACCACATTGG AACTGACCTG AAGAAGCAAA CTCGGAAAAT | 2400 |
| ATAATAACAT CCCTGAATTC AGGCATTCAC AAGATGCAGA ACAAAATGGA TAAAAGGTAT | 2460 |
| TTCACTGGAG AAGTTTTAAT TTCTAAGTAA AATTTAAATC CTAACACTTC ACTAATTTAT | 2520 |
| AACTAAAATT TCTCATCTTC GTACTTGATG CTCACAGAGG AAGAAAATGA TGATGGTTTT | 2580 |
| TATTCCTGGC ATCCAGAGTG ACAGTGAACT TAAGCAAATT ACCCTCCTAC CCAATTCTAT | 2640 |
| GGAATATTTT ATACGTCTCC TTGTTTAAAA TCTGACTGCT TTACTTTGAT GTATCATATT | 2700 |
| TTTAAATAAA AATAAATATT CCTTTAGAAG ATCACTCTAA AA | |

Seq ID NO: 16 Protein sequence:
Protein Accession #: NP_004096

| | |
|---|---|
| 1 11 21 31 41 51 | |
| \| \| \| \| \| \| | |
| MLKALFLTML TLALVKSQDT EETITYTQCT DGYEWDPVRQ QCKDIDECDI VPDACKGGMK | 60 |
| CVNHYGGYLC LPKTAQIIVN NEQPQQETQP AEGTSGATTG VVAASSMATS GVLPGGGFVA | 120 |
| SAAAVAGPEM QTGRNNFVIR RNPADPQRIP SNPSHRIQCA AGYEQSEHNV CQDIDECTAG | 180 |
| THNCRADQVC INLRGSFACQ CPPGYQKRGE QCVDIDECTI PPYCHQRCVN TPGSFYCQCS | 240 |
| PGFQLAANNY TCVDINECDA SNQCAQQCYN ILGSFICQCN QGYELSSDRL NCEDIDECRT | 300 |
| SSYLCQYQCV NEPGKFSCMC PQGYQVVRSR TCQDINECET TNECREDEMC WNYHGGFRCY | 360 |
| PRNPCQDPYI LTPENRCVCP VSNAMCRELP QSIVYKYMSI RSDRSVPSGI FQIQATTIYA | 420 |
| NTINTFRIKS GNENGEFYLR QTSPVSAMLV LVKSLSGPRE HIVDLEMLTV SSIGTFRTSS | 480 |
| VLRLTIIVGP FSF | |

Seq ID NO: 17 Nucleotide sequence:
Nucleic Acid Accession #: NM_018894
Coding sequence: 27 . . . 1967 (underlined sequences correspond to
start and stop codons)

| | |
|---|---|
| 1 11 21 31 41 51 | |
| \| \| \| \| \| \| | |
| AAAACATTCA ACAAATTAAT GGGTGTAAGG AACTGGAAAA CCTGGACTCC TACCACATGC | 60 |
| AGATAAAACC AATAGAGTGC AGAATAAGAC TCAAGTCAAG TAAGTAACGT TAAACACCAT | 120 |
| AAAGACACAT GGCCTTCTTT GTGTACATGA CATGCATTCT CAACAATGCA CTGACGGATA | 180 |
| TGAGTGGGAT CCTGTGAGAC AGCAATGCAA AGATATTGAT GAATGTGACA TTGTCCCAGA | 240 |
| CGCTTGTAAA GGTGGAATGA AGTGTGTCAA CCACTATGGA GGATACCTCT GCCTTCCGAA | 300 |
| AACAGCCCAG ATTATTGTCA ATAATGAACA GCCTCAGCAG GAAACACAAC CAGCAGAAGG | 360 |
| AACCTCAGGG GCAACCACCG GGGTTGTAGC TGCCAGCAGC ATGGCAACCA GTGGAGTGTT | 420 |
| GCCCGGGGGT GGTTTTGTGG CCAGTGCTGC TGCAGTCGCA GGCCCTGAAA TGCACACTGG | 480 |
| CCGAAATAAC TTTGTCATCC GGCGGAACCC AGCTGACCCT CAGCGCATTC CCTCCAACCC | 540 |
| TTCCCACCGT ATCCAGTGTG CAGCAGGCTA CGAGCAAAGT GAACACAACG TGTGCCAAGA | 600 |
| CATAGACGAG TGCACTCCAG GGACGCACAA CTGTAGAGCA GACCAAGTGT GCATCAATTT | 660 |
| ACGGGGATCC TTTGCATGTC AGTGCCCTCC TGGATATCAG AAGCGAGGGG AGCAGTGCGT | 720 |
| AGACATAGAT GAATGTACCA TCCCTCCATA TTGCCACCAA AGATGCGTGA ATACACCAGG | 780 |
| CTCATTTTAT TGCCAGTGCA GTCCTGGGTT TCAATTGGCA GCAAACAACT ATACCTGCGT | 840 |
| AGATATAAAT GAATGTGATG CCAGCAATCA ATGTGCTCAG CAGTGCTACA ACATTCTTGG | 900 |
| TTCATTCATC TGTCAGTGCA ATCAAGGATA TGAGCTAAGC AGTGACAGGC TCAACTGTGA | 960 |
| AGACATTGAT GAATGCAGAA CCTCAAGCTA CCTGTGTCAA TATCAATGTG TCAATGAACC | 1020 |
| TGGGAAATTC TCATGTATGT GCCCCCAGGG ATACCAAGTG GTGAGAAGTA GAACATGTCA | 1080 |
| AGATATAAAT GAGTGTGAGA CCACAAATGA ATGCCGGGAG GATGAAATGT GTTGGAATTA | 1140 |
| TCATGGCGGC TTCCGTTGTT ATCCACGAAA TCCTTGTCAA GATCCCTACA TTCTAACACC | 1200 |
| AGAGAACCGA TGTGTTTGCC CAGTCTCAAA TGCCATGTGC CGAGAACTGC CCCAGTCAAT | 1260 |
| AGTCTACAAA TACATGAGCA TCCGATCTGA TAGGTCTGTG CCATCAGACA TCTTCCAGAT | 1320 |
| ACAGGCCACA ACTATTTATG CCAACACCAT CAATACTTTT CGGATTAAAT CTGGAAATGA | 1380 |
| AAATGGAGAG TTCTACCTAC GACAAACAAG TCCTGTAAGT GCAATGCTTG TGCTCGTGAA | 1440 |
| GTCATTATCA GGACCAAGAG AACATATCGT GGACCTGGAG ATGCTGACAG TCAGCAGTAT | 1500 |
| AGGGACCTTC CGCACAAGCT CTGTGTTAAG ATTGACAATA ATAGTGGGGC CATTTTCATT | 1560 |
| TTAGTCTTTT CTAAGAGTCA ACCACAGGCA TTTAAGTCAG CCAAAGAATA TTGTTACCTT | 1620 |
| AAAGCACTAT TTTATTTATA GATATATCTA GTGCATCTAC ATCTCTATAC TGTACACTCA | 1680 |
| CCCATAACAA ACAATTACAC CATGGTATAA AGTGGGCATT TAATATGTAA AGATTCAAAG | 1740 |
| TTTGTCTTTA TTACTATATG TAAATTAGAC ATTAATCCAC TAAACTGGTC TTCTTCAAGA | 1800 |
| GAGCTAAGTA TACACTATCT GGTGAAACTT GGATTCTTTC CTATAAAAGT GGGACCAAGC | 1860 |
| AATGATGATC TTCTGTGGTG CTTAAGGAAA CTTACTAGAG CTCCACTAAC AGTCTCATAA | 1920 |
| GGAGGCAGCC ATCATAACCA TTGAATAGCA TGCAAGGGTA AGAATGAGTT TTTAACTGCT | 1980 |
| TTGTAAGAAA ATGGAAAAGG TCAATAAAGA TATATTTCTT TAGAAAATGG GGATCTGCCA | 2040 |
| TATTTGTGTT GGTTTTTATT TTCATATCCA GCCTAAAGGT GGTTGTTTAT TATATAGTAA | 2100 |
| TAAATCATTG CTGTACAACA TGCTGGTTTC TGTAGGGTAT TTTTAATTTT GTCAGAAATT | 2160 |
| TTAGATTGTG AATATTTTGT AAAAAACAGT AAGCAAAATT TTCCAGAATT CCCAAAATGA | 2220 |
| ACCAGATACC CCCTAGAAAA TTATACTATT GAGAAATCTA TGGGGAGGAT ATGAGAAAAT | 2280 |
| AAATTCCTTC TAAACCACAT TGGAACTGAC CTGAAGAAGC AAACTCGGAA AATATAATAA | 2340 |
| CATCCCTGAA TTCAGGCATT CACAAGATGC AGAACAAAAT GGATAAAAGG TATTTCACTG | 2400 |
| GAGAAGTTTT AATTTCTAAG TAAAATTTAA ATCCTAACAC TTCACTAATT TATAACTAAA | 2460 |
| ATTTCTCATC TTCGTACTTG ATGCTCACAG AGGAAGAAAA TGATGATGGT TTTTATTCCT | 2520 |
| GGCATCCAGA GTGACAGTGA ACTTAAGCAA ATTACCCTCC TACCCAATTC TATGGAATAT | 2580 |
| TTTATACGTC TCCTTGTTTA AAATCTGACT GCTTTACTTT GATGTATCAT ATTTTTAAAT | 2640 |
| AAAAATAAAT ATTCCTTTAG AAGATCACTC TAAAA | |

Seq ID No: 18 Protein sequence:
Protein Accession #: NP_061489.1

| | |
|---|---|
| 1 11 21 31 41 51 | |
| \| \| \| \| \| \| | |
| MHSQQCTDGY EWDPVRQQCK DIDECDIVPD ACKGGMKCVN HYGGYLCLPK TAQIIVNNEQ | 60 |
| PQQETQPAEG TSGATTGVVA ASSMATSGVL PGGGFVASAA AVAGPEMQTG RNNFVIRRNP | 120 |
| ADPQRIPSNP SHRIQCAAGY EQSEHNVCQD IDECTAGTHN CRADQVCINL RGSFACQCPP | 180 |
| GYQKRGEQCV DIDECTIPPY CHQRCVNTPG SFYCQCSPGF QLAANNYTCV DINECDASNQ | 240 |
| CAQQCYNILG SFICQCNQGY ELSSDRLNCE DIDECRTSSY LCQYQCVNEP GKFSCMCPQG | 300 |
| YQVVRSRTCQ DINECETTNE CREDEMCWNY HGGFRCYPRN PCQDPYILTP ENRCVCPVSN | 360 |
| AMCRELPQSI VYKYMSIRSD RSVPSDIFQI QATTIYANTI NTFRIKSGNE NGEFYLRQTS | 420 |
| PVSAMLVLVK SLSGPREHIV DLEMLTVSSI GTFRTSSVLR LTIIVGPFSF | |

Seq ID NO: 19 Nucleotide sequence:
Nucleic Acid Accession #: NM_006500
Coding sequence: 27 . . . 1967 (underlined sequences correspond to
start and stop codons)

| | |
|---|---|
| 1 11 21 31 41 51 | |
| \| \| \| \| \| \| | |
| ACTTGCGTCT CGCCCTCCGG CCAAGCATGG GGCTTCCCAG GCTGGTCTGC GCCTTCTTGC | 60 |
| TCGCCGCCTG CTGCTGCTGT CCTCGCGTCG CGGGTGTGCC CGGAGAGGCT GAGCAGCCTG | 120 |
| CGCCTGAGCT GGTGGAGGTG GAAGTGGGCA GCACAGCCCT TCTGAAGTGC GGCCTCTCCC | 180 |
| AGTCCCAAGG CAACCTCAGC CATGTCGACT GGTTTTCTGT CCACAAGGAG AAGCGGACGC | 240 |
| TCATCTTCCG TGTGCGCCAG GGCCAGGGCC AGAGCGAACC TGGGGAGTAC GAGCAGCGGC | 300 |
| TCAGCCTCCA GGACAGAGGG GCTACTCTGG CCCTGACTCA AGTCACCCCC CAAGACGAGC | 360 |
| GCATCTTCTT GTGCCAGGGC AAGCGCCCTC GGTCCCAGGA GTACCGCATC CAGCTCCGCG | 420 |
| TCTACAAAGC TCCGGAGGAG CCAAACATCC AGGTCAACCC CCTGGGCATC CCTGTGAACA | 480 |
| GTAAGGAGCC TGAGGAGGTC GCTACCTGTG TAGGGAGGAA CGGGTACCCC ATTCCTCAAG | 540 |
| TCATCTGGTA CAAGAATGGC CGGCCTCTGA AGGAGGAGAA GAACCGGGTC CACATTCAGT | 600 |
| CGTCCCAGAC TGTGGAGTCG AGTGGTTTGT ACACCTTGCA GAGTATTCTG AAGGCACAGC | 660 |
| TGGTTAAAGA AGACAAAGAT GCCCAGTTTT ACTGTGAGCT CAACTACCGG CTGCCCAGTG | 720 |
| GGAACCACAT GAAGGAGTCC AGGGAAGTCA CCGTCCCTGT TTTCTACCCG ACAGAAAAAG | 780 |
| TGTCGCTGGA AGTGGAGCCC CTGGGAATGC TGAAGCAAGG GGACCGCGTG GAAATCAGGT | 840 |
| GTTTGGCTGA TGGCAACCCT CCACCACACT TCAGCATCAG CAAGCAGAAC CCCAGCACCA | 900 |
| GGGAGGCAGA GGAAGAGACA ACCAACGACA ACGGGGTCCT GGTGCTGGAG CCTGCCCGGA | 960 |
| AGGAACACAG TGGGCGCTAT GAATGTCAGG CCTGGAACTT GGACACCATG ATATCGCTGC | 1020 |
| TGAGTGAACC ACAGGAACTA CTGGTGAACT ATGTGTCTGA CGTCCGAGTG AGTCCCGCAG | 1080 |
| CCCCTGAGAG ACAGGAAGGC AGCAGCCTCA CCCTGACCTG TGAGGCAGAG AGTAGCCAGG | 1140 |
| ACCTCGAGTT CCAGTGGCTG AGAGAAGAGA CAGACCAGGT GCTGGAAAGG GGGCCTGTGC | 1200 |
| TTCAGTTGCA TGACCTGAAA CGGGAGGCAG GAGGCGGCTA TCGCTGCGTG GCGTCTGTGC | 1260 |
| CCAGCATACC CGGCCTGAAC CGCACACAGC TGGTCAAGCT GGCCATTTTT GGCCCCCCTT | 1320 |
| GGATGGCATT CAAGGAGAGG AAGGTGTGGG TGAAAGAGAA TATGGTGTTG AATCTGTCTT | 1380 |
| GTGAAGCGTC AGGGCACCCC CGGCCCACCA TCTCCTGGAA CGTCAACGGC ACGGCAAGTG | 1440 |
| AACAAGACCA AGATCCACAG CGAGTCCTGA GCACCCTGAA TGTCCTCGTG ACCCCGGAGC | 1500 |
| TGTTGGAGAC AGGTGTTGAA TGCACGGCCT CCAACGACCT GGGCAAAAAC ACCAGCATCC | 1560 |
| TCTTCCTGGA GCTGGTCAAT TTAACCACCC TCACACCAGA CTCCAACACA ACCACTGGCC | 1620 |
| TCAGCACTTC CACTGCCAGT CCTCATACCA GAGCCAACAG CACCTCCACA GAGAGAAAGC | 1680 |
| TGCCGGAGCC GGAGAGCCGG GGCGTGGTCA TCGTGGCTGT GATTGTGTGC ATCCTGGTCC | 1740 |
| TGGCGGTGCT GGGCGCTGTC CTCTATTTCC TCTATAAGAA GGGCAAGCTG CCGTGCAGGC | 1800 |
| GCTCAGGGAA GCAGGAGATC ACGCTGCCCC CGTCTCGTAA GACCGAACTT GTAGTTGAAG | 1860 |
| TTAAGTCAGA TAAGCTCCCA GAAGAGATGG GCCTCCTGCA GGGCAGCAGC GGTGACAAGA | 1920 |
| GGGCTCCGGG AGACCAGGGA GAGAAATACA TCGATCTGAG GCATTAGCCC CGAATCACTT | 1980 |
| CAGCTCCCTT CCCTGCCTGG ACCATTCCCA GCTCCCTGCT CACTCTTCTC TCAGCCAAAG | 2040 |
| CCTCCAAAGG GACTAGAGAG AAGCCTCCTG CTCCCCTCAC CTGCACACCC CCTTTCAGAG | 2100 |
| GGCCACTGGG TTAGGACCTG AGGACCTCAC TTGGCCCTGC AAGCCGCTTT TCAGGGACCA | 2160 |
| GTCCACCACC ATCTCCTCCA CGTTGAGTGA AGCTCATCCC AAGCAAGGAG CCCCAGTCTC | 2220 |
| CCGAGCGGGT AGGAGAGTTT CTTGCAGAAC GTGTTTTTTC TTTACACACA TTATGGCTGT | 2280 |
| AAATACCTGG CTCCTGCCAG CAGCTGAGCT GGGTAGCCTC TCTGAGCTGG TTTCCTGCCC | 2340 |
| CAAAGGCTGG CTTCCACCAT CCAGGTGCAC CACTCAAGTG AGGACACACC GGAGCCAGGC | 2400 |
| GCCTGCTCAT GTTGAAGTGC GCTGTTCACA CCCGCTCCGG AGAGCACCCC AGCGGCATCC | 2460 |
| AGAAGCAGCT GCAGTGTTGC TGCCACCACC CTCCTGCTCG CCTCTTCAAA GTCTCCTGTG | 2520 |
| ACATTTTTTC TTTGGTCAGA AGCCAGGAAC TGGTGTCATT CCTTAAAAGA TACGTGCCGG | 2580 |
| GGCCAGGTGT GGTGGCTCAC GCCTGTAATC CCAGCACTTT GGGAGGCCGA GGCGGGCGGA | 2640 |
| TCACAAAGTC AGGACGAGAC CATCCTGGCT AACACGGTGA AACCCTGTCT CTACTAAAAA | 2700 |
| TACAAAAAAA AATTAGCTAG GCGTAGTGGT TGGCACCTAT AGTCCCAGCT ACTCGGAAGG | 2760 |
| CTGAAGCAGG AGAATGGTAT GAATCCAGGA GGTGGAGCTT GCAGTGAGCC GAGACCGTGC | 2820 |
| CACTGCACTC CAGCCTGGGC AACACAGCGA GACTCCGTCT CGAGGAAAAA AAAAGAAAAG | 2880 |
| ACGCGTACCT GCGGTGAGGA AGCTGGGCGC TGTTTTCGAG TTCAGGTGAA TTAGCCTCAA | 2940 |
| TCCCCGTGTT CACTTGCTCC CATAGCCCTC TTGATGGATC ACGTAAAACT GAAAGGCAGC | 3000 |
| GGGGACCAGA CAAAGATGAG GTCTACACTG TCCTTCATGG GGATTAAAGC TATGGTTATA | 3060 |
| TTAGCACCAA ACTTCTACAA ACCAAGCTCA GGGCCCCAAC CCTAGAAGGG CCCAAATGAG | 3120 |
| AGAATGGTAC TTAGGGATGG AAAACGGGGC CTGGCTAGAG CTTCGGGTGT GTGTGTCTGT | 3180 |
| CTGTGTGTAT GCATACATAT GTGTGTATAT ATGGTTTTGT CAGGTGTGTA AATTTGCAAA | 3240 |
| TTGTTTCCTT TATATATGTA TGTATATATA TATATGAAAA TATATATATA TATGAAAAAT | 3300 |
| AAAGCTTAAT TGTCCCAGAA AATCATACAT TGCTTTTTTA TTCTACATGG GTACCACAGG | 3360 |
| AACCTGGGGG CCTGTGAAAC TACAACCAAA AGGCACACAA AACCGTTTCC AGTTGGCAGC | 3420 |
| AGAGATCAGG GGTTACCTCT GCTTCTGAGC AAATGGCTCA AGCTCTACCA GAGCAGACAG | 3480 |
| CTACCCTACT TTTCAGCAGC AAAACGTCCC GTATGACGCA GCACGAAGGG CCTGGCAGGC | 3540 |
| TGTTAGCAGG AGCTATGTCC CTTCCTATCG TTTCCGTCCA CTT | |

Seq ID NO: 20 Protein sequence:
Protein Accession #: NP_006491

| | |
|---|---|
| 1 11 21 31 41 51 | |
| \| \| \| \| \| \| | |
| MGLPRLVCAF LLAACCCCPR VAGVPGEAEQ PAPELVEVEV GSTALLKCGL SQSQGNLSHV | 60 |
| DWFSVHKEKR TLIFRVRQGQ GQSEPGEYEQ RLSLQDRGAT LALTQVTPQD ERIFLCQGKR | 120 |
| PRSQEYRIQL RVYKAPEEPN IQVNPLGIPV NSKEPEEVAT CVGRNGYPIP QVIWYKNGRP | 180 |
| LKEEKNRVHI QSSQTVESSG LYTLQSILKA QLVKEDKDAQ FYCELNYRLP SGNHMKESRE | 240 |
| VTVPVFYPTE KVWLEVEPVG MLKEGDRVEI RCLADGNPPP HFSISKQNPS TREAEEETTN | 300 |
| DNGVLVLEPA RKEHSGRYEC QAWNLDTMIS LLSEPQELLV NYVSDVRVSP AAPERQEGSS | 360 |
| LTLTCEAESS QDLEFQWLRE ETDQVLERGP VLQLHDLKRE AGGGYRCVAS VPSIPGLNRT | 420 |
| QLVKLAIFGP PWMAFKERKV WVKENMVLNL SCEASGHPRP TISWNVNGTA SEQDQDPQRV | 480 |
| LSTLNVLVTP ELLETGVECT ASNDLGKNTS ILFLELVNLT TLTPDSNTTT GLSTSTASPH | 540 |
| TRANSTSTER KLPEPESRGV VIVAVIVCIL VLAVLGAVLY FLYKKGKLPC RRSGKQEITL | 600 |
| PPSRKTELVV EVKSDKLPEE MGLLQGSSGD KRAPGDQGEK YIDLRH | |

Seq ID NO: 21 Nucleotide sequence:
Nucleic Acid Accession #: NM_002421
Coding sequence: 72 . . . 1481 (underlined sequences correspond to
start and stop codons)

| | |
|---|---|
| 1 11 21 31 41 51 | |
| \| \| \| \| \| \| | |
| GGGATATTGG AGTAGCAAGA GGCTGGGAAG CCATCACTTA CCTTGCACTG AGAAAGAAGA | 60 |
| CAAAGGCCAG TATGCACAGC TTTCCTCCAC TGCTGCTGCT GCTGTTCTGG GGTGTGGTGT | 120 |
| CTCACAGCTT CCCAGCGACT CTAGAAACAC AAGAGCAAGA TGTGGACTTA GTCCAGAAAT | 180 |
| ACCTGGAAAA ATACTACAAC CTGAAGAATG ATGGGAGGCA AGTTGAAAAG CGGAGAAATA | 240 |
| GTGGCCCAGT GGTTGAAAAA TTGAAGCAAA TGCAGGAATT CTTTGGGCTG AAAGTGACTG | 300 |
| GGAAACCAGA TGCTGAAACC CTGAAGGTGA TGAAGCAGCC CAGATGTGGA GTGCCTGATG | 360 |
| TGGCTCAGTT TGTCCTCACT GAGGGGAACC CTCGCTGGGA GCAAACACAT CTGACCTACA | 420 |
| GGATTGAAAA TTACACGCCA GATTTGCCAA GAGCAGATGT GGACCATGCC ATTGAGAAAG | 480 |
| CCTTCCAACT CTGGAGTAAT GTCACACCTC TGACATTCAC CAAGGTCTCT GAGGGTCAAG | 540 |
| CAGACATCAT GATATCTTTT GTCAGGGGAG ATCATCGGGA CAACTCTCCT TTTGATGGAC | 600 |
| CTGGAGGAAA TCTTGCTCAT GCTTTTCAAC CAGGCCCAGG TATTGGAGGG GATGCTCATT | 660 |
| TTGATGAAGA TGAAAGGTGG ACCAACAATT TCAGAGAGTA CAACTTACAT CGTGTTGCGG | 720 |
| CTCATGAACT CGGCCATTCT CTTGGACTCT CCCATTCTAC TGATATCGGG GCTTTGATGT | 780 |
| ACCCTAGCTA CACCTTCAGT GGTGATGTTC AGCTAGCTCA GGATGACATT GATGGCATCC | 840 |
| AAGCCATATA TGGACGTTCC CAAAATCCTG TCCAGCCCAT CGGCCCACAA ACCCCAAAAG | 900 |
| CGTGTGACAG TAAGCTAACC TTTGATGCTA TAACTACGAT TCGGGGAGAA GTGATGTTCT | 960 |
| TTAAAGACAG ATTCTACATG CGCACAAATC CCTTCTACCC GGAAGTTGAG CTCAATTTCA | 1020 |
| TTTCTGTTTT CTGGCCACAA CTGCCAAATG GGCTTGAAGC TGCTTACGAA TTTGCCGACA | 1080 |
| GAGATGAAGT CCGGTTTTTC AAAGGGAATA ACTACTGGGC TGTTCAGGGA CAGAATGTGC | 1140 |
| TACACGGATA CCCCAAGGAC ATCTACAGCT CCTTTGGCTT CCCTAGAACT GTGAAGCATA | 1200 |
| TCGATGCTGC TCTTTCTGAG GAAAACACTG GAAAAACCTA CTTCTTTGTT GCTAACAAAT | 1260 |
| ACTGGAGGTA TGATGAATAT AAACGATCTA TGGATCCAGG TTATCCCAAA ATGATAGCAC | 1320 |
| ATGACTTTCC TGGAATTGGC CACAAAGTTG ATGCAGTTTT CATGAAAGAT GGATTTTTCT | 1380 |
| ATTTCTTTCA TGGAACAAGA CAATACAAAT TTGATCCTAA AACGAAGAGA ATTTTGACTC | 1440 |
| TCCAGAAAGC TAATAGCTGG TTCAACTGCA GGAAAAATTG AACATTACTA ATTTGAATGG | 1500 |
| AAAACACATG GTGTGAGTCC AAAGAAGGTG TTTTCCTGAA GAACTGTCTA TTTTCTCAGT | 1560 |
| CATTTTTAAC CTCTAGAGTC ACTGATACAC AGAATATAAT CTTATTTATA CCTCAGTTTG | 1620 |
| CATATTTTTT TACTATTTAG AATGTAGCCC TTTTTGTACT GATATAATTT AGTTCCACAA | 1680 |
| ATGGTGGGTA CAAAAAGTCA AGTTTGTGGC TTATGGATTC ATATAGGCCA GAGTTGCAAA | 1740 |
| GATCTTTTCC AGAGTATGCA ACTCTGACGT TGATCCCAGA GAGCAGCTTC AGTGACAAAC | 1800 |
| ATATCCTTTC AAGACAGAAA GAGACAGGAG ACATGAGTCT TTGCCGGAGG AAAAGCAGCT | 1860 |
| CAAGAACACA TGTGCAGTCA CTGGTGTCAC CCTGGATAGG CAAGGGATAA CTCTTCTAAC | 1920 |
| ACAAAATAAG TGTTTTATGT TTGGAATAAA GTCAACCTTG TTTCTACTGT TTT | |

Seq ID NO: 22 Protein sequence:
Protein Accession #: NP_002412

| | |
|---|---|
| 1 11 21 31 41 51 | |
| \| \| \| \| \| \| | |
| MHSFPPLLLL LFWGVVSHSF PATLETQEQD VDLVQKYLEK YYNLKNDGRQ VEKRRNSGPV | 60 |
| VEKLKQMQEF FGLKVTGKPD AETLKVMKQP RCGVPDVAQF VLTEGNPRWE QTHLTYRIEN | 120 |
| YTPDLPRADV DHAIEKAFQL WSNVTPLTFT KVSEGQADIM ISFVRGDHRD NSPFDGPGGN | 180 |
| LAHAFQPGPG IGGDAHFDED ERWTNNFREY NLHRVAAHEL GHSLGLSHST DIGALMYPSY | 240 |
| TFSGDVQLAQ DDIDGIQAIY GRSQNPVQPI GPQTPKACDS KLTFDAITTI RGEVMFFKDR | 300 |
| FYMRTNPFYP EVELNFISVF WPQLPNGLEA AYEFADRDEV RFFKGNKYWA VQGQNVLHGY | 360 |
| PKDIYSSFGF PRTVKHIDAA LSEENTGKTY FFVANKYWRY DEYKRSMDPG YPKMIAHDFP | 420 |
| GIGHKVDAVF MKDGFFYFFH GTRQYKFDPK TKRILTLQKA NSWFNCRKN | |

Seq ID NO: 23 Nucleotide sequence:
Nucleic Acid Accession #: FGENESH predicted ORF
Coding sequence: 141-1580 (underlined sequences correspond to start
and stop codons)

| | |
|---|---|
| 1 11 21 31 41 51 | |
| \| \| \| \| \| \| | |
| TCTGCGTGTG CCGGGGCTAG GGGCTGGAAG TCCTGGCTCT AGTTGCACCT CGGAAGGAAA | 60 |
| AGGCAAACAG AGGAGGGAAG GCGTCTTAGG ACTGCCTGGA TCCAGAGCAC TTTCCTCGGC | 120 |
| CTCTACAGGC CTGTGTCGCT ATGGGTTCCC CCGCCGCCCC GGAGGGAGCG CTGGGCTACG | 180 |
| TCCGCGAGTT CACTCGCCAC TCCTCCGACG TGCTGGGCAA CCTCAACGAG CTGCGCCTGC | 240 |
| GCGGGATCCT CACTGACGTC ACGCTGCTGG TTGGCGGGCA ACCCCTCAGA GCACACAAGG | 300 |
| CAGTTCTCAT CGCCTGCAGT GGCTTCTTCT ATTCAATTTT CCGGGGCCGT GCGGGAGTCG | 360 |
| GGGTGGACGT GCTCTCTCTG CCCGGGGGTC CCGAAGCGAG AGGCTTCGCC CCTCTATTGG | 420 |
| ACTTCATGTA CACTTCGCGC CTGCGCCTCT CTCCAGCCAC TGCACCAGCA GTCCTAGCGG | 480 |
| CCGCCACCTA TTTGCAGATG GAGCACGTGG TCCAGGCATG CCACCGCTTC ATCCAGGCCA | 540 |
| GCTATGAACC TCTGGGCATC TCCCTGCGCC CCCTGGAAGC AGAACCCCCA ACACCCCCAA | 600 |
| CGGCCCCTCC ACCAGGTAGT CCCAGGCGCT CCGAAGGACA CCCAGACCCA CCTACTGAAT | 660 |
| CTCGAAGCTG CAGTCAAGGC CCCCCCAGTC CAGCCAGCCC TGACCCCAAG GCCTGCAACT | 720 |
| GGAAAAAGTA CAAGTACATC GTGCTAAACT CTCAGGCCTC CCAAGCAGGG AGCCTGGTCG | 780 |
| GGGACAGAAG TTCTGGTCAA CCTTGCCCCC AAGCCAGGCT CCCCAGTGGA GACGAGGCCT | 840 |
| CCAGCAGCAG CAGCAGCAGC AGCAGCAGCA GTGAAGAAGG ACCCATTCCT GGTCCCCAGA | 900 |
| GCAGGCTCTC TCCAACTGCT GCCACTGTGC AGTTCAAATG TGGGGCTCCA GCCAGTACCC | 960 |
| CCTACCTCCT CACATCCCAG GCTCAAGACA CCTCTGGATC ACCCTCTGAA CGGGCTCGTC | 1020 |
| CACTACCGGG AAGTGAATTT TTCAGCTGCC AGAACTGTGA GGCTGTGGCA GGGTGCTCAT | 1080 |
| CGGGGCTGGA CTCCTTGGTT CCTGGGGACG AAGACAAACC CTATAAGTGT CAGCTGTGCC | 1140 |
| GGTCTTCGTT CCGCTACAAG GGCAACCTTG CCAGTCATCG TACAGTGCAC ACAGGGGAAA | 1200 |
| AGCCTTACCA CTGCTCAATC TGCGGAGCCC GTTTTAACCG GCCAGCAAAC CTGAAAACGC | 1260 |
| ACAGCCGCAT CCATTCGGGA GAGAAGCCGT ATAAGTGTGA GACGTGCGGC TCGCGCTTTG | 1320 |
| TACAGGTGGC ACATCTGCGG GCGCACGTGC TGATCCACAC CGGGGAGAAC CCCTACCCTT | 1380 |
| GCCCTACCTG CGGAACCCGC TTCCGCCACC TGCAGACCCT CAAGAGCCAC GTTCGCATCC | 1440 |
| ACACCGGAGA GAAGCCTTAC CACTGCGACC CCTGTGGCCT GCATTTCCGG CACAAGAGTC | 1500 |
| AACTGCGGCT GCATCTGCGC CAGAAACACG GAGCTGCTAC CAACACCAAA GTGCACTACC | 1560 |
| ACATTCTCGG GGGGCCCTAG CTGAGCGCAG GCCCAGGCCC CACTTGCTTC CTGCGGGTGG | 1620 |
| GAAAGCTGCA GGCCCAGGCC TTGCTTCCCT ATCAGGCTTG GGCATAGGGG TGTGCCAGGC | 1680 |
| CACTTTGGTA TCAGAAATTG CCACCCTCTT AATTTCTCAC TGGGGAGAGC AGGGGTGGCA | 1740 |
| GATCCTGGCT AGATCTGCCT CTGTTTTGCT GGTCAAAACC TCTTCCCCAC AAGCCAGATT | 1800 |
| GTTTCTGAGG AGAGAGCTAG CTAGGGGCTG GGAAAGGGGA GAGATTGGAG TCCTGGTCTC | 1860 |
| CCTAAGGGAA TAGCCCTCCA CCTGTGGCCC CCATTGCATT CAGTTTATCT GTAAATATAA | 1920 |
| TTTATTGAGG CCTTTGGGTG GCACCGGGGC CTTCATTCGA TTGCATTTCC CACTCCCCTC | 1980 |
| TTCCACAAGT GTGATTAAAA GTGACCAGAA ACACAGAAGG TGAGATCACA GCTCTGCTGG | 2040 |
| CAGAGATTAC TAGCCCTTGG CTCTCTCGTT TGGCTTGGGT ATTTTATATT ATTTCTGTCA | 2100 |
| TAACTTTTAT CTTTAGAATT GTTCTTTCTC CTGTTTGTTT GCTTGTTAGT TTGTTTAAAA | 2160 |
| TGGAAAAAGG GGTTCTCTGT GTTCTGCCCC TGTAATTCTA GGTCTGGAAC CTTTATTTGT | 2220 |
| TCTAGGGCAG CTCTGGGAAC ATGCGGGATT GTGGAATTGG GTCAGGAACC CTCTCTGGTA | 2280 |
| TTCTGGATGT TGTAGGTTCT CTAGCAGTCT AGAAATGGAT ACAGACATTT CTCTGTTCTT | 2340 |
| CAAGGGTGAT AGGAACCATT ATGTTGAGCC CAAAATGGAA GTAATAATAA ATGCCTCCTG | 2400 |
| GAGGCTGTGG GTGTGGGGGA TTCTGTATCT GGATTCCGTA TCACTCCAAC TGGAGGCTGT | 2460 |
| GGGTGTGGGG GATTCTGTAT CTGGATTCCG TATCACTCCA AGTGGAGGCT GGCAGGTTTT | 2520 |
| TCTGCAAGAT GGTCCAGAAT CTAAAATGTC CCATTAATCT GGTCACTTGG GTTTGGCTCT | 2580 |
| GCTGTATCCA TCTATAGTGG TAGAGACCCA CCAGGGCTCA AGTGGAGTCC ATCATCCTCC | 2640 |
| CACGGGGGCC TGTTCTTAGC ACTGAGTTGA TCGCTCCATG GGGGAGAGAT CAGACATTCC | 2700 |
| TTATCAGAGA TGATGTGACC TTTTCTGACT CTGCCCAGTC TCTATGAATG TTATGGCCTA | 2760 |
| GGGAAGAATC ATGAAACTCT TTAGCTTGAT TAGATGGTAA ACAGTGTTAA CCCATCCTTT | 2820 |
| ACTACAGAGG CATATGGGTT TGAATGTTAC CTGGGGTTCT CTCTATTGAG TTGAGCCCCT | 2880 |
| TCTTCCTTTA GTGGGTTTTG GACATCTTCT GGCAAGTGTC CAGATGCCAG AACCTTCTTT | 2940 |
| TCCTCTAGAA GGGATGGTGC TTGGTAACCT TACCTTTTAA AAGCTGGGTC TGTGACCTGG | 3000 |
| TCTTCCCATC CCTGCATTCC TGTCTGGAAC CAGTGAATGC ATTAGAACCT TCCATAGGAA | 3060 |
| AAGAAAAGGG GCTGAGTTCC ATTCTGGGTT TGCTGTAGTT TGGTTGGGAT TATTGTTGGC | 3120 |
| ATTACAGATG TAAAAGATTG ACTAGCCCAT AGGCCAAAGG CCTGTTCTAG TTGACCAAGT | 3180 |
| TTCAAGTAGG ATTAAGAGGT TGGTTGAGGG GTGCAGTTTC TGGTGTAGGC CAGGTAGGTA | 3240 |
| GAAAGTGAGG AACAGGGTTG CCTCTTGGCT GGGTGGAGTC TCTGAAATGT TAGAAGAAGC | 3300 |
| GCTGAAGCCT TGATTGATAG TTCTGCCCCT TGTTGCCCTG GGGCTTATCT GATTATGGGA | 3360 |
| CGAGGGTAGA AAGTAAGAAG CACTTTTGAA TTTGTGGGGT AGAACTTCAA CAATAAGTCA | 3420 |
| GTTCTAGTGG CTGTCGCCTG GGGACTAGTG AGAAAGCTAC TCTTCTCCCT CTTCCCTCTT | 3480 |
| TCTCCCCATG GCCCCACTGC AGAATTAAAG AAGGAAGAAG GGAAGGCGGA GGAGTCTATA | 3540 |
| AGAAGGAATC ATGATTTCTA TTTAGCAGAT TGGATGGGCA GGTGGAGAAT GCCTGGGGGT | 3600 |
| AGAAATGTTA GATCTTGCAA CATCAGATCC TTGGAATAAA GAAGCCTCTC TGYGCWRAAA | 3660 |
| AAAAAAAAAA AAAAAA | |

Seq ID NO: 24 Protein sequence:
Protein Accession #: FGENESH predicted

| | |
|---|---|
| 1 11 21 31 41 51 | |
| \| \| \| \| \| \| | |
| MGSPAAPEGA LGYVREFTRH SSDVLGNLNE LRLRGILTDV TLLVGGQPLR AHKAVLIACS | 60 |
| GFFYSIFRGR AGVGVDVLSL PGGPEARGFA PLLDFMYTSR LRLSPATAPA VLAAATYLQM | 120 |
| EHVVQACHRF IQASYEPLGI SLRPLEAEPP TPPTAPPPGS PRRSEGHPDP PTESRSCSQG | 180 |
| PPSPASPDPK ACNWKKYKYI VLNSQASQAG SLVGERSSGQ PCPQARLPSG DEASSSSSSS | 240 |
| SSSSEEGPIP GPQSRLSPTA ATVQFKCGAP ASTPYLLTSQ AQDTSGSPSE RARPLPGSEF | 300 |
| FSCQNCEAVA GCSSGLDSLV PGDEDKPYKC QLCRSSFRYK GNLASHRTVH TGEKPYHCSI | 360 |
| CGARFNRPAN LKTHSRIHSG EKPYKCETCG SRFVQVAHLR AHVLIHTGEK PYPCPTCGTR | 420 |
| FRHLQTLKSH VRIHTGEKPY HCDPCGLHFR HKSQLRLHLR QKHGAATNTK VHYHILGGP | |

Seq ID NO: 25 Nucleotide sequence:
Nucleic Acid Accession #: U21551
Coding sequence: 1 . . . 1155 (underlined sequences correspond to
start and stop codons)

| | |
|---|---|
| 1 11 21 31 41 51 | |
| \| \| \| \| \| \| | |
| ATGGATTGCA GTAACGGATC GGCAGAGTGT ACCGGAGAAG GAGGATCAAA AGAGGTGGTG | 60 |
| GGGACTTTTA AGGCTAAAGA CCTAATAGTC ACACCAGCTA CCATTTTAAA GGAAAAACCA | 120 |
| GACCCCAATA ATCTGGTTTT TGGAACTGTG TTCACGGATC ATATGCTGAC GGTGGAGTGG | 180 |
| TCCTCAGAGT TTGGATGGGA GAAACCTCAT ATCAAGCCTC TTCAGAACCT GTCATTGCAC | 240 |
| CCTGGCTCAT CAGCTTTGCA CTATGCAGTG GAATTATTTG AAGGATTGAA GGCATTTCGA | 300 |
| GGAGTAGATA ATAAAATTCG ACTGTTTCAG CCAAACCTCA ACATGGATAG AATGTATCGC | 360 |
| TCTGCTGTGA GGGCAACTCT GCCGGTATTT GACAAAGAAG AGCTCTTAGA GTGTATTCAA | 420 |
| CAGCTTGTGA AATTGGATCA AGAATGGGTC CCATATTCAA CATCTGCTAG TCTGTATATT | 480 |
| CGTCCTGCAT TCATTGGAAC TGAGCCTTCT CTTGGAGTCA AGAAGCCTAC CAAAGCCCTG | 540 |
| CTCTTTGTAC TCTTGAGCCC AGTGGGACCT TATTTTTCAA GTGGAACCTT TAATCCAGTG | 600 |
| TCCCTGTGGG CCAATCCCAA GTATGTAAGA GCCTGGAAAG GTGGAACTGG GGACTGCAAG | 660 |
| ATGGGAGGGA ATTACGGCTC ATCTCTTTTT GCCCAATGTG AAGACGTAGA TAATGGGTGT | 720 |
| CAGCAGGTCC TGTGGCTCTA TGGCACAGAC CATCAGATCA CTGAAGTGGG AACTATGAAT | 780 |
| CTTTTTCTTT ACTGGATAAA TGAAGATGGA GAAGAAGAAC TGGCAACTCC TCCACTAGAT | 840 |
| GGCATCATTC TTCCAGGAGT GACAAGGCGG TGCATTCTGG ACCTGGCACA TCAGTGGGGT | 900 |
| GAATTTAAGG TGTCAGAGAG ATACCTCACC ATGGATGACT TGACAACAGC CCTGGAGGGG | 960 |
| AACAGAGTGA GAGAGATGTT TAGCTCTGGT ACAGCCTGTG TTGTTTGCCC AGTTTCTGAT | 1020 |
| ATACTGTACA AAGGCGAGAC AATACACATT CCAACTATGG AGAATGGTCC TAAGCTGGCA | 1080 |
| AGCCGCATCT TGAGCAAATT AACTGATATC CAGTATGGAA GAGAAGAGAG CGACTGGACA | 1140 |
| ATTGTGCTAT CCTGA | |

Seq ID NO: 26 Protein sequence:
Protein Accession #: AAB08528

| | |
|---|---|
| 1 11 21 31 41 51 | |
| \| \| \| \| \| \| | |
| MDCSNGSAEC TGEGGSKEVV GTFKAKDLIV TPATILKEKP DPNNLVFGTV FTDHMLTVEW | 60 |
| SSEFGWEKPH IKPLQNLSLH PGSSALHYAV ELFEGLKAFR GVDNKIRLFQ PNLNMDRMYR | 120 |
| SAVRATLPVF DKEELLECIQ QLVKLDQEWV PYSTSASLYI RPAFIGTEPS LGVKKPTKAL | 180 |
| LFVLLSPVGP YFSSGTFNPV SLWANPKYVR AWKGGTGDCK MGGNYGSSLF AQCEDVDNGC | 240 |
| QQVLWLYGRD HQITEVGTMN LFLYWINEDG EEELATPPLD GIILPGVTRR CILDLAHQWG | 300 |
| EFKVSERYLT MDDLTTALEG NRVREMFSSG TACVVCPVSD ILYKGETIHI PTMENGPKLA | 360 |
| SRILSKLTDI QYGREESDWT IVLS | |

Seq ID NO: 27 Nucleotide sequence:
Nucleic Acid Accession #: XM_039209
Coding sequence: 656 . . . 2758 (underlined sequences correspond to
start and stop codons)

| | |
|---|---|
| 1 11 21 31 41 51 | |
| \| \| \| \| \| \| | |
| TCGCGCGGGG GCCGCCCCCT CCCCTTCCCT CCACCCTGGG CGGGGGCGCG CGAGAAGCGG | 60 |
| TGACGTCAAG GGGCGCGCTG TCGCAGCACC TCCCCGCGCG CTAGTTAAAA AGAAGAAGAA | 120 |
| AAGAGGGAAC GAAACATGAG AGGCTGTGTG AGAAGCTGCA GCCGCCGGCA GAGGAGACCT | 180 |
| CAGCATCATC TAGAGCCCAG CGCTGGCCCT GCCTCCGCCT GCCCCGCCGC CGCCGTCGCC | 240 |
| GTTTCTGTTC CTGCTACTGT CCCACCTAAA CAACTCCCGT TACACGGACA AGTGAACATC | 300 |
| TGTGGCTGTC CTCTCCTTTT CTTCCTCCTC TTCCAACTCC TTCTCCTCCT CCCACTTCCC | 360 |
| AGCCGCAGCA GAAAGCCCCC AACCCAACTG ACACTGGCAC AACTGCAAAC GGTGTCATCC | 420 |
| GCACAACTTT ATCTCGCTCC TCGGGCTCCC CTAAGGCATT GGACCCATCG CCGCGTCTTT | 480 |
| TATTTTTTGC AAAGTTGCAT CGCTGTACAT ATTTTTGTCC CCGCCACCTC CCTCTGTCTC | 540 |
| TGGAGTGCCC TACAGCCCCG CAAACTCCTC CTGGAGCTGC GCCCTAGTGC CCCTGCTGGG | 600 |
| CAGTGGCGTT CCCCCCCATC CTCCCGCGCC CAGCCCCTGC TGCTCTGGGC AGACGATGCT | 660 |
| GAAGATGCTC TCCTTTAAGC TGCTGCTGCT GGCCGTGGCT CTGGGCTTCT TTGAAGGAGA | 720 |
| TGCTAAGTTT GGGGAAAGAA ACGAAGGGAG CGGAGCAAGG AGGAGAAGGT GCCTGAATCC | 780 |
| GAACCCCCCG AAGCGCCTGA AAAGGAGAGA CAGGAGGATG ATGTCCCAGC TGGAGCTGCT | 840 |
| GAGTGGGGGA GAGATGCTGT GCGGTGGCTT CTACCCTCGG CTGTCCTGCT GCCTGCGGAG | 900 |
| TGACAGCCCG GGGCTAGGGC GCCTGGAGAA TAAGATATTT TCTGTTACCA ACAACACAGA | 960 |
| ATGTGGGAAG TTACTGGAGG AAATCAAATG TGCACTTTGC TCTCCACATT CTCAAAGCCT | 1020 |
| GTTCCACTCA CCTGAGAGAG AAGTCTTGGA AAGAGACCTA GTACTTCCTC TGCTCTGCAA | 1080 |
| AGACTATTGC AAAGAATTCT TTTACACTTG CCGAGGCCAT ATTCCAGGTT TCCTTCAAAC | 1140 |
| AACTGCGGAT GAGTTTTGCT TTTACTATGC AAGAAAAGAT GGTGGGTTGT GCTTTCCAGA | 1200 |
| TTTTCCAAGA AAACAAGTCA GAGGACCAGC ATCTAACTAC TTGGACCAGA TGGAAGAATA | 1260 |
| TGACAAAGTG GAAGAGATCA GCAGAAAGCA CAAACACAAC TGCTTCTGTA TTCAGGAGGT | 1320 |
| TGTGAGTGGG CTGCGGCAGC CCGTTGGTGC CCTGCATAGT GGGGATGGCT CGCAACGTCT | 1380 |
| CTTCATTCTG GAAAAAGAAG GTTATGTGAA GATACTTACC CCTGAAGGAG AAATTTTCAA | 1440 |
| GGAGCCTTAT TTGGACATTC ACAAACTTGT TCAAAGTGGA ATAAAGGGAG GAGATGAAAG | 1500 |
| AGGACTGCTA AGCCTCGCAT TCCATCCCAA TTACAAGAAA AATGGAAAGT TGTATGTGTC | 1560 |
| CTATACCACC AACCAAGAAC GGTGGGCTAT CGGGCCTCAT GACCACATTC TTAGGGTTGT | 1620 |
| GGAATACACA GTATCCAGAA AAAATCCACA CCAAGTTGAT TTGAGAACAG CCAGAGTCTT | 1680 |
| TCTTGAAGTT GCAGAACTCC ACAGAAAGCA TCTGGGAGGA CAACTGCTCT TTGGCCCTGA | 1740 |
| CGGCTTTTTG TACATCATTC TTGGTGATGG GATGATTACA CTGGATGATA TGGAAGAAAT | 1800 |
| GGATGGGTTA AGTGATTTCA CAGGCTCAGT GCTACGGCTG GATGTGGACA CAGACATGTG | 1860 |
| CAACGTGCCT TATTCCATAC CAAGGAGCAA CCCACACTTC AACAGCACCA ACCAGCCCCC | 1920 |
| CGAAGTGTTT GCTCATGGGC TCCACGATCC AGGCAGATGT GCTGTGGATA GACATCCCAC | 1980 |
| TGATATAAAC ATCAATTTAA CGATACTGTG TTCAGACTCC AATGGAAAAA ACAGATCATC | 2040 |
| AGCCAGAATT CTACAGATAA TAAAGGGGAA AGATTATGAA AGTGAGCCAT CACTTTTAGA | 2100 |
| ATTCAAGCCA TTCAGTAATG CTCCTTTGGT TGGTGGATTT GTATACCGGG GCTGCCAGTC | 2160 |
| AGAAAGATTG TATGGAAGCT ACGTGTTTGG AGATCGTAAT GGGAATTTCC TAACTCTCCA | 2220 |
| GCAAAGTCCT GTGACAAAGC AGTGGCAAGA AAAACCACTC TGTCTCGGCA CTAGTGGGTC | 2280 |
| CTGTACAGGC TACTTTTCCG GTCACATCTT GGGATTTGGA GAACATGAAC TAGGTGAAGT | 2340 |
| TTACATTTTA TCAAGCAGTA AAAGTATGAC CCAGACTCAC AATGGAAAAC TCTACAAAAT | 2400 |
| TGTAGATCCC AAAAGACCTT TAATGCCTGA GGAATGCAGA GCCACGGTAC AACCTGCACA | 2460 |
| GACACTGACT TCAGAGTGCT CCAGGCTCTG TCGAAACGGC TACTGCACCC CCACGGCAAA | 2520 |
| GTGCTGCTGC ACTCCAGGCT GGGAGGGGGA CTTCTGCAGA ACTGCAAAAT GTGAGCCAGC | 2580 |
| ATGTCGTCAT GGAGGTGTCT GTGTTAGACC GAACAAGTGC CTCTGTAAAA AAGGATATCT | 2640 |
| TGGTCCTCAA TGTGAACAAG TGCACACAAA CATCCCCACA GTGACCACGC CAGGTATTCT | 2700 |
| TGATCAGATC ATTGACATCA CATCTTACTT GCTGGATCTA ACAAGTTACA TTGTATAGTT | 2760 |
| TCTGGGACTG TTTGAATATT CTATTCCAAT GGGCATTTAT TTTTTATCCT CTCATTAAAA | 2820 |
| AAAAAAGACT GTTATCCTGC TACACACTCC TGTGATTTCA TTCTCTTTTA TTAATTTAAA | 2880 |
| AATAATTTCC ACAAATGTGC AGATCCTCTG TGTGTATGTC AGCATGTTTC TTCACATATG | 2940 |
| CACATACACA TACTCATAAC CCCTATATGC GTTCTTCCAT AACACATGAT TTTTTAAAAT | 3000 |
| ATATACTTCC TTATGCAAAG TAATTTACAC AGAAATTCCA TTGTAAATTG ATAATGGATT | 3060 |
| TTTTATGTTA CTAGAAGAGA TTATTTGACT TCCCAGGAAT TTTCTGTCTG TAATCACTAA | 3120 |
| AGTCAACTTT AATACACTTT TGAAACAGTA CTGTGCAATC CGATGGATCT AATTAAAAAA | 3180 |
| AAGGCAATAT TTTTATATTA AAGTACTATA CTAGGAGAGA ATGTTTCAGA ACTCCCTGAT | 3240 |
| GAATTTCTAA GTGAGCAACT TGATATAAAA TTGTAATCTT CATTTTTGTC AGTGTATCCA | 3300 |
| GTTACAGAAT GCTACACACT TACCTTTTTA TTGGCTGACA AATCTGCTTA TTTCATCTTA | 3360 |
| ATCTCAACAT TGTTTTCAAG TGTTTTATAA TTAAATCATA ATAGCATATT TTAAAATCAA | 3420 |
| TCTTCCTAAA AGGTCTGCTT TTATTGTATA TTTTATTTAA CAATAGGCAC TGCGTTTGTG | 3480 |
| TTACATATTT ATATATTTTA TTTTATTTTT ATAATATAGA CATCACCTAG | |

Seq ID NO: 28 Protein sequence:
Protein Accession #: XP_039209

| | |
|---|---|
| 1 11 21 31 41 51 | |
| \| \| \| \| \| \| | |
| MLKMLSFKLL LLAVALGFFE GDAKFGERNE GSGARRRRCL NGNPPKRLKR RDRRMMSQLE | 60 |
| LLSGGEMLCG CFYPRLSCCL RSDSPGLGRL ENKIFSVTNN TECGKLLEEI KCALCSPHSQ | 120 |
| SLFHSPEREV LERDLVLPLL CKDYCKEFFY TCRGHIPGFL QTTADEFCFY YARKDGGLCF | 180 |
| PDFPRKQVRG PASNYLDQME EYDKVEEISR KHKNNCFCIQ EVVSCLRQPV CALHSGDGSQ | 240 |
| RLFILEKEGY VKILTPEGEI FKEPYLDIHK LVQSGIKGGD ERGLLSLAFH PNYKKNGKLY | 300 |
| VSYTTNQERW AIGPHDHILR VVEYTVSRKN PHQVDLRTAR VFLEVAELHR KHLGCQLLFG | 360 |
| PDGFLYIILG DGMITLDDME EMDGLSDFTG SVLRLDVDTD MCNVPYSIPR SNPHFNSTNQ | 420 |
| PPEVFAHGLH DPGRCAVDRH PTDTNINLTI LCSDSNGKNR SSARILQIIK GKDYESEPSL | 480 |
| LEFKPFSNGP LVGGFVYRGC QSERLYGSYV FGDRNGNFLT LQQSPVTKQW QEKPLCLGTS | 540 |
| GSCRGYFSGH ILGFGEDELG EVYILSSSKS MTQTHNGKLY KIVDPKRPLM PEECRATVQP | 600 |
| AQTLTSECSR LCRNGYCTPT GKCCCSPGWE GDFCRTAKCE PACRHGGVCV RPNKCLCKKG | 660 |
| YLGPQCEQVD RNIRRVTRAG ILDQIIDMTS YLLDLTSYIV | |

Seq ID NO: 29 Nucleotide sequence:
Nucleic Acid Accession #: NM_024756
Coding sequence: 75. . . 2924 (underlined sequences correspond to
start and stop codons)

| | |
|---|---|
| 1 11 21 31 41 51 | |
| \| \| \| \| \| \| | |
| AAGACAACGT CACTAGCAGT TTCTGGAGCT ACTTGCCAAG GCTGAGTGTG AGCTGAGCCT | 60 |
| GCCCCACCAC CAACATCATC CTGAGCTTGC TGTTCAGCCT TGGGGGCCCC CTGCGCTCGG | 120 |
| GGCTGCTGGG GGCATGGCCC CAGGCTTCCA GTACTAGCCT CTCTGATCTG CACAGCTCCA | 180 |
| GGACACCTGG GGTCTGGAAG CCAGAGGCTG AGGACACCAG CAAGGACCCC GTTGGACGTA | 240 |
| ACTGGTGCCC CTACCCAATG TCCAAGCTGG TCACCTTACT AGCTCTTTGC AAAACAGAGA | 300 |
| AATTCCTCAT CCACTCGCAG CAGCCGTGTC CGCAGGGAGC TCCAGACTGC CAGAAAGTCA | 360 |
| AAGTCATGTA CCGCATGGCC CACAAGCCAG TGTACCAGGT CAAGCAGAAG GTGCTGACCT | 420 |
| CTTTGGCCTG GAGGTGCTGC CCTGGCTACA CGGGCCCCAA CTGCGAGCAC CACGATTCCA | 480 |
| TGGCAATCCC TGAGCCTGCA GATCCTGGTG ACAGCCACCA GGAACCTCAG GATGGACCAG | 540 |
| TCAGCTTCAA ACCTGCCCAC CTTGCTGCAG TGATCAATGA GGTTGAGGTG CAACAGGAAC | 600 |
| AGCAGGAACA TCTGCTGGGA GATCTCCAGA ATGATGTGCA CCGGGTGGCA GACAGCCTGC | 660 |
| CAGGCCTGTG GAAAGCCCTG CCTGGTAACC TCACAGCTGC AGTGATGGAA GCAAATCAAA | 720 |
| CAGGGCACGA GTTCCCTGAT AGATCCTTGG AGCAGGTGCT GCTACCCCAC GTGGACACCT | 780 |
| TCCTACAAGT GCATTTCAGC CCCATCTGGA GGAGCTTTAA CCAAAGCCTG CACAGCCTTA | 840 |
| CCCAGGCCAT AAGAAACCTG TCTCTTGACG TGGAGGCCAA CCGCCAGGCC ATCTCCAGAG | 900 |
| TCCAGGACAG TGCCGTGGCC AGGGCTGACT TCCAGGAGCT TGGTGCCAAA TTTGAGGCCA | 960 |
| AGGTCCAGGA GAACACTCAG AGAGTGGGTC AGCTGCGACA GGACGTGGAG GACCGCCTGC | 1020 |
| ACGCCCAGCA CTTTACCCTG CACCGCTCGA TCTCAGACCT CCAAGCCGAT GTGGACACCA | 1080 |
| AATTGAAGAG GCTGCACAAG GCTCAGGAGG CCCCAGGGAC CAATGGCAGT CTGGTGTTGG | 1140 |
| CAACGCCTGG GGCTGGGGCA AGGCCTGAGC CGGACAGCCT GCAGGCCAGG CTGGGCCAGC | 1200 |
| TGCAGACGAA CCTCTCAGAG CTGCACATGA CCACGGCCCG CAGGGAGGAG GAGTTGCAGT | 1260 |
| ACACCCTGGA GGACATGAGG GCCACCCTGA CCCGGCACGT GGATGAGATC AAGGAACTGT | 1320 |
| ACTCCGAATC GGACGAGACT TTCGATCAGA TTAGCAAGGT GGAGCGGCAG GTGGAGGAGC | 1380 |
| TGCAGGTGAA CCACACGGCG CTCCGTGAGC TGCGCGTGAT CCTCATGGAG AAGTCTCTGA | 1440 |
| TCATCCAGGA GAACAAGCAG CAGCTGGAGC CGCAGCTCCT GGAGCTCAAC CTCACGCTGC | 1500 |
| AGCACCTGCA CGGTCGCCAT GCCGACCTCA TCAAGTACGT GAAGGACTGC AATTGCCAGA | 1560 |
| AGCTCTATTT AGACCTGGAC GTCATCCGGG AGGGCCAGAG GGACGCCACG CGTGCCCTGG | 1620 |
| AGGAGACCCA GGTGAGCCTG GACGAGCGGC GGCAGCTGGA CGGCTCCTCC CTGCAGGCCC | 1680 |
| TGCAGAACGC CGTGGACGCC GTGTCCCTCC CCGTGCACCC CCACAAAGCG CAGCGCGAGC | 1740 |
| GGGCGCGGGC GGCCACGTCG CCGCTCCCGA GCCAAGTGCA GGCGCTGGAT GACGAGGTGG | 1800 |
| GCGCGCTGAA GGCGGCCGCG GCCGAGGCCC GCCACGAGGT GCGCCAGCTG CACAGCGCCT | 1860 |
| TCGCCGCCCT GCTGCACCAC GCGCTGCGGC ACGAGGCGGT GCTGGCCGCG CTCTTCGGGG | 1920 |
| AGGAGGTGCT GGAGGAGATG TCTGAGCAGA CGCCGGGACC CCTGCCCCTG AGCTACGAGC | 1980 |
| AGATCCGCGT GGCCCTCCAG GACGCCGCTA GCGCGCTGCA GGAGCAGGCG CTCGCCTCGC | 2040 |
| ACGAGCTCGC CGCCCGAGTG ACGGCCCTGG AGCAGGCCTC GCAGCCCCCG CGGCCGGCAG | 2100 |
| AGCACCTGGA GCCCAGCCAC GACGCGGGCC GCGAGGAGGC CGCCACCACC GCCCTGGCCG | 2160 |
| GGCTGGCGCG GGAGCTCCAG ACCCTCAGCA ACGACGTCAA CAATGTCGGG CGGTGCTGCG | 2220 |
| AGGCCGAGGC CGGGGCCGGG GCCGCCTCCC TCAACGCCTC CCTTGACGGC CTCCACAACG | 2280 |
| CACTCTTCGC CACTCAGCGC AGCTTGGAGC AGCACCAGCG GCTCTTCCAC AGCCTCTTTG | 2340 |
| GGAACTTCCA AGGGCTCATG GAAGCCAACG TCAGCCTGCA CCTGCGGAAG CTGCAGACCA | 2400 |
| TGCTGAGCAG GAAAGGCAAG AAGCAGCACA AAGACCTGGA AGCTCCCCGG AACAGGGACA | 2460 |
| AGAAGGAAGC GGAGCCTTTG GTGGACATAC GGGTCACAGG GCCTGTGCCA GGTGCCTTGG | 2520 |
| GCGCCGCGCT CTGCGAGGCA GGATCCCCTG TCGCCTTCTA TGCCACCTTT TCAGAACGGA | 2580 |
| CGGCTGCCCT GCAGACAGTG AACTTCAACA CCACATACAT CAACATTGGC AGCAGCTACT | 2640 |
| TCCCTCAACA TCGCTACTTC CCAGCCCCTG AGCGTGGTCT CTACCTGTTT GCAGTGACCG | 2700 |
| TTGAATTTGG CCCAGGCCCA GGCACCGCGC AGCTGGTGTT TGGAGGTCAC CATCGGACTC | 2760 |
| CAGTCTGTAC CACTGCGCAG GGGAGTGGAA GCACAGCAAC GGTCTTTGCC ATGGCTGAGC | 2820 |
| TGCAGAAGGG TGAGCGAGTA TGGTTTGAGT TAACCCAGGG ATCAATAACA AAGAGAAGCC | 2880 |
| TGTCGGGCAC TGCATTTGGG GGCTTCCTGA TGTTTAAGAC CTGAACCCCA GCCCCAATCT | 2940 |
| GATCAGACAT CATGGACTCG CCCAGCTCTC CTCGGCCTGG CCCTCTGGCC AAGGATGGGC | 3000 |
| TGGAGGTCAT TCAGTTGGTC TGTCTCTTCC CTGGAAACCT TCTGCAAAGA TGGTGTGGTG | 3060 |
| TACGTGGCTT CCCTGTAACC ACATGGGGCT TGGCCATTTC TCCATGATGA GAAGGACTGG | 3120 |
| AATGCTTCTC CGGGCAGGAC ATGGTCCTAG GAAGCCTGAA CCTTGGCTTG GCATGCCTTC | 3180 |
| TCAGACAGCA CGGCCTGGGC TCCAACTCTT CACCACACCC TGTATTCTAC AACTTCTTTG | 3240 |
| GTGTTTTGCT CCTCCTGTGG TTGGAAACTT CTGTACAACA CTTTAAACTT TTCTCTTGCT | 3300 |
| TCCTCTTCTC TTCTCCCTTA TCGTATGATA GAAAGACATT CTTCCCCAGG AGGAATGTTT | 3360 |
| AAAATGGAGG CAACATTTTG GCCAACATTG GAAAGCACTA GAGGGCAATG GGATTAAACC | 3420 |
| AACCTGCTTG GTCTCTATTA GTCAGTAATG AAGACGACAG CCTGGCCAAC CAAGGGAAAG | 3480 |
| GAAATTAGTA TCTTTAGTTT CAGTCATTCC TTGTAGGATA TGGTTTAGCT GTGCCCCCAC | 3540 |
| CTAAAATATC ATCTTGAATT GTAATCCCTA TAATCCCCAC ATCAAGGGAG AGATCAGGTG | 3600 |
| GAGGTAATTG GATCTTGGGG GCGGTTCCCC CATGCTGTTC TTGTGATAGT TCTCACGAGA | 3660 |
| TCTGATGATT TTATAAGTTT GATAGTTCCT CCTGTGTTCA TTCTCCTTCC TGCCACCTTG | 3720 |
| TGAAGATGCC TTGGTTCCTC TTCACTGTCT GCCATGATTG TAAGTTTCCT GAGGCCTCCC | 3780 |
| CAGCCATGTG GAACAGTGAG TCAATTAAAC CTCTTTCCTT TATAAATT | |

Seq ID NO: 30 protein sequence:
Protein Accession #: NP_079032

| | |
|---|---|
| 1 11 21 31 41 51 | |
| \| \| \| \| \| \| | |
| MILSLLFSLG GPLGWGLLGA WAQASSTSLS DLQSSRTPGV WKAEAEDTSK DPVGRNWCPY | 60 |
| PMSKLVTLLA LCKTEKFLIH SQQPCPQGAP DCQKVKVMYR MAHKPVYQVK QKVLTSLAWR | 120 |
| CCPGYTGPNC EHHDSMAIPE PADPGDSHQE PQDGPVSFKP GHLAAVINEV EVQQEQQENL | 180 |
| LGDLQNDVHR VADSLPGLWK ALPGNLTAAV MEANQTGHEF PDRSLEQVLL PNVDTFLQVH | 240 |
| FSPIWRSFNQ SLHSLTQAIR NLSLDVEANR QAISRVQDSA VARADFQELG AKFEAKVQEN | 300 |
| TQRVGQLRQD VEDRLHAQHF TLHRSISELQ ADVDTKLKRL HKAQEAPGTN GSLVLATPGA | 360 |
| GARPEPDSLQ ARLGQLQRNL SELHMTTARR EEELQYTLED MRATLTRHVD EIKELYSESG | 420 |
| ETFDQISKVE RQVEELQVNH TALRELRVIL MEKSLIMEEN KEEVERQLLE LNLTLQHLQG | 480 |
| GHADLIKYVK DCNCQKLYLD LDVIREGQRD ATRALEETQV SLDERRQLDG SSLQALQNAV | 540 |
| DAVSLAVDAH KAEGERARAA TSRLRSQVQA LGDEVGALKA AAAEARHEVR QLHSAEAALL | 600 |
| EDALRHEAVL AALFGEEVLE EMSEQTPGPL PLSYEQIRVA LQDAASGLQE QALGWDELAA | 660 |
| RVTALEQASE PPRPAEHLEP SHDAGREEAA TTALAGLARE LQSLSNDVKN VGRCCEAEAG | 720 |
| AGAASLNASL DGLHNALFAT QRSLEQHQRL FHSLFGNFQG LMEANVSLDL GKLQTMLSRK | 780 |
| GKKQQKDLEA PRKRDKKEAE PLVDIRVTGP VPGALGAALW EAGSPVAFYA SFSEGTAALQ | 840 |
| TVKFNTTYIN IGSSYFPEHG YFRAPERGVY LFAVSVEFGP GPGTGQLVFG GHHRTPVCTT | 900 |
| GQGSGSTATV FAMAELQKGE RVWFELTQGS ITKRSLSGTA FGGFLMFKT | |

Seq ID NO: 31 Nucleotide sequence:
Nucleic Acid Accession #: AB037715
Coding sequence: 370 . . . 3489 (underlined sequences correspond to
start and stop codons)

| | |
|---|---|
| 1 11 21 31 41 51 | |
| \| \| \| \| \| \| | |
| GAACGCTCAC AGAACAGGCA GTGCAATTCC ATGTTCCTCT TAAGTATGTT AGCCCTACCG | 60 |
| GGAGCTGAGC TGGCCAGTCT ACTTGGAGAG GAAAAGTAGA TCTGGGGAAG GTGGAAGGGT | 120 |
| CAGTTCCTAA GTGACTTCCT CCTCGGGGAT GGTAAGGGCA TTTGCTGATC TCCAGTGACT | 180 |
| GCCTGGTGCC TCATGGTCAG ACTCGGCTGT CTCACTCCCA GATATCTGAT TTTGCAAAAA | 240 |
| GGGACACACC TATCTGCAGC AAAGAAGACA CTGACCAGAT TGCGAGCGGT GCTTTTGGAT | 300 |
| GCTCTGTAGC CACCCGGGGC CCAGGAGGAC TGACTCGGCA GCAGGATTCG TGCATGGGAA | 360 |
| TCGGAGACCA TGGCAGTGCA GCTGGTGCCC GACTCAGCTC TCCGCCTGCT GATGATCACG | 420 |
| GAGGGCCGCC GATGTCAAGT ACATCTTCTT GATGACAGGA AGCTGGAACT CCTAGTACAC | 480 |
| CCCAAGCTGT TGGCCAAGGA GCTTCTTGAC CTTGTGGCTT CTCACTTCAA TCTGAAGGAA | 540 |
| AAGGAGTACT TTGGAATAGC ATTCACACAT GAAACGGGAC ACTTAAACTG GCTTCAGCTA | 600 |
| GATCCAAGAG TATTGGAACA TGACTTCCCT AAAAAGTCAG GACCCGTGGT TTTATACTTT | 660 |
| TGTGTCAGGT TCTATATAGA AAGCATTTCA TACCTGAAGC ATAATGCTAC CATTGAGCTT | 720 |
| TTCTTTCTGA ACGCGAAGTC CTGCATCTAC AAGGAGCTTA TTGACCTTCA CAGCGAAGTC | 780 |
| GTGTTTGAAT TAGCTTCCTA TATTTTACAG GAGGCAAAGG GAGATTTTTC TAGCAATGAA | 840 |
| GTTGTGAGGA GTGACTTGAA GAAGCTGCCA GCCCTTCCCA CCCAACCCCT CAACGACCAC | 900 |
| CCTTCCCTGG CCTACTGTGA AGACAGACTC ATTGACCACT ACAAGAAACT GAACGGTCAG | 960 |
| ACAAGAGGTC AAGCAATCGT AAACTACATG ACCATCGTCG AGTCTCTCCC AACCTACGGG | 1020 |
| GTTCACTATT ATGCAGTGAA GGACAAGCAG GGCATACCAT GGTGGCTGCG CCTGAGCTAC | 1080 |
| AAAGGGATCT TCCAGTATGA CTACCATGAT AAAGTCAACC CAAGAAACAT ATTCCAATGG | 1140 |
| AGACAGTTGG AAAACCTGTA CTTCAGAGAA AAGAAGTTTT CCGTGGAAGT TCATGACCCA | 1200 |
| CGCAGGGCTT CAGTGACAAG CAGGACGTTT GGGCACAGCG GCATTGCAGT GCACACGTGC | 1260 |
| TATGCATGTC CCGCATTGAT CAAGTCCATC TGGGCTATGG CCATAAGCCA ACACCAGTTC | 1320 |
| TATCTGGACA GAAAGCAGAG TAAGTCCAAA ATCCATGCAG CACGCAGCCT GAGTGAGATC | 1380 |
| GCCATCGACC TGACCGAGAC GGGGACGCTG AAGACCTCGA AGCTGGCCAA CATGGCTAGC | 1440 |
| AAGGCGAAGA TCATCAGCGG CACCAGCCGC AGCCTGCTGT CTTCAGGTTC TCAGGAATCA | 1500 |
| GATAGCTCGC ACTCCCCCAA GAAGGACATG CTGGCTCCCT TGAAGTCCAG GCAGGAAGCT | 1560 |
| CTGGAGGAAA CCCTGCGTCA GAGGCTGGAG GAACTGAAGA AGCTGTGTCT CCGAGAAGCT | 1620 |
| GAGCTCACGG GCAAGCTGCC AGTAGAATAT CCCCTCGATC CAGGGGAGCA ACCACCCATT | 1680 |
| GTTCGGAGAA GAATAGGAAC AGCCTTCAAA CTGGATGAAC ACAAAATCCT GCCCAAAGGA | 1740 |
| GAGGAAGCTG ACCTCGAACG CCTGGAACGA GAGTTTGCCA TTCAGTCCCA GATTACGGAG | 1800 |
| GCCGCCCGCC GCCTAGCCAG TGACCCCAAC GTCACCAAAA AACTGAAGAA ACAAAGGAAA | 1860 |
| ACCTCGTATC TGAATGCACT GAAGAAACTC CACGAGATTG AAAATGCAAT CAATGACAAC | 1920 |
| CGCATCAACT CTGGGAAGAA ACCCACCCAG AGGGCTTCGC TGATCATAGA CGATGGAAAC | 1980 |
| ATTGCCAGTG AAGACAGCTC CCTCTCAGAT GCCCTTGTTC TTGAGCATGA AGACTCTCAG | 2040 |
| GTTACCAGCA CAATATCCCC CCTACATTCT CCTCACAAGG GACTCCCTCC TCGGCCACCG | 2100 |
| TCGCACAACA GGCCTCCTCC TCCCCAGTCC CTGGAGCGAC TCCGACACAT CCACTATCAC | 2160 |
| CGCAACGACT ATGACAAGTC ACCCATCAAG CCCAAAATGT GGAGTGAGTC CTCTTTAGAT | 2220 |
| GAACCCTATG AGAAGGTCAA GAAGCGCTCC TCTCACAGCC ATTCCAGCAG CCACAACCGC | 2280 |
| TTCCCCAGCA CAGGAAGCTG TCCGGAAGCC GCCCCAGCAA GCAACTCCTT GCAGAACAGC | 2340 |
| CCCATCCGCG GCCTCCCGCA CTGGAACTCC CAGTCCAGCA TGCCGTCCAC GCCAGACCTG | 2400 |
| CGGGTCCGGA GTCCCCACTA CGTCCATTCC ACGAGGTCGG TCGACATCAG CCCCACCCGA | 2460 |
| CTGCACAGCC TCGCACTGCA CTTTACGCAC CGGAGCTCCA GCCTGGAGTC CCAGCGCAAG | 2520 |
| CTCCTGGGCT CCGAAAACGA CACCGGCACC CCCGACTTCT ACACCCCGCG GACTCGTAGC | 2580 |
| AGCAACGGCT CAGACCCCAT GGACGACTGC TCGTCGTGCA CCAGCCACTC GAGCTCGCAG | 2640 |
| CACTACTACC CGGCGCAGAT GAACGCCAAC TACTCCACGC TCGCCGAGGA CTCGCCGTCC | 2700 |
| AAGGCGCGCC AGAGGCAGAG GCAGCGGCAG CGGGCGGCGG GCGCACTGGG CTCAGCCAGC | 2760 |
| TCGGGCACCA TGCCCAACCT GGCCGCGCGC GGGGGTGCGG GGGCCGCGGG GGGCGCGGCC | 2820 |
| GGCGGTGTGT ACCTCCACAG CCAGAGCCAG CCCAGCTCGC AGTACCGCAT CAAGGAGTAC | 2880 |
| CCGCTGTACA TCCACGGCCG CGCCACGCCC GTGGTGGTGC GCAGCCTGGA GACCGACCAG | 2940 |
| GAGTGCCACT ACAGCGTCAA CGCTCAGTTC AAGACGTCCA ACTCCTACAC GGCGGCCGGC | 3000 |
| CTGTTCAAGG AGACCTGCCG CCGCGGCGGC GGCGACGAGG GCGACACGGG CCGCCTGACG | 3060 |
| CCGTCGCGAT CGCAGATCCT GCGGACTCCG TCGCTGGGCC GCGAGGGCGC CCACGACAAG | 3120 |
| GGCGCGGGCC GTGCCGCCGT CTCAGACGAG CTGCGCCACT GGTACCAGCG TTCCACCGCC | 3180 |
| TCGCACAAGG AGCACAGCCC CCTGTCCCAC ACCAGCTCCA CCTCCTCGGA CAGCGGCTCG | 3240 |
| CAGTACAGCA CCTCCTCCCA GACCACCTTC GTGGCGCACA GCAGGCTCAC CAGGATGCCC | 3300 |
| CAGATGTGCA ACGCCACGTC AGCTGCCTTA CCTCAAACCC AGAGAAGCTC GACACCCTCA | 3360 |
| AGTGAAATTG GAGCCACCCC CCCAAGCAGC CCCCACCACA TCCTAACCTG CCACACTGGA | 3420 |
| GAAGCAACAG AAAACTCACC CATTCTCCAT CCCTCTGAGT CTCCACCTCA CCAAAGTACT | 3480 |
| GATGAATAGA GGAGCTACAA TGATAGCTGT TTCCTGGATT CCTCCCTCTA TCCAGAACTA | 3540 |
| GCTCATGTCC AGTCCTACGG GCAGGAAAAA GCCAAGCCCG GGACCCTCCT GTCAGCCACC | 3600 |
| CCGGCCTAAT CTGACCGCCT CAACGCCATT CTGAGATCAC CTCACTGCCT CTCATTTGCC | 3660 |
| TTACCCAGAC GCACCGTCAC CCTCCACCAG CTTTGGCCCT CAGCACTTTT TTTCTCCTGT | 3720 |
| CTCCGCATTC CCTCCCCCTT GAAAACCTGA CTGACGACAC ATTCTGGAAC GTTCCGGTCC | 3780 |
| CACTGTGTGT CCCCTGGCGC TCTTCCCCAT AGAGAGCCAG ACACCAATCC TCAATGGCAC | 3840 |
| CTTGGTGGCT TCCCTCTGCC ATCACAGCCC CTAGCCCACC AACCATCAGG CGGGCCAGCC | 3900 |
| GGCATCCAAT TCCTGCGGAT AAGTAGCGTT GCCAGAGAAC GGGAAAGGGG ACTTCGGTTA | 3960 |
| CAGGGTGACC CAGAAAGACG ATTCAGCTGT GTCCAGCCTG CCACCCATAC GTAGGCCAAC | 4020 |
| CAAGCACTTC ATGAAGAGGA CCCCTCGTCC CATATTCAGT TTACACCTGA AATATTCCTT | 4080 |
| GATGGGACAC CTTGTGGGGA TCGCTATGGG GGAAGGGGAG CTTGAGAAAG GAACTTCTCC | 4140 |
| ACACCAGAAA TGCATCGGAG GACCACAATC AGTTCTATCC TGCCAAACAT TAAAAATAAA | 4200 |
| TAAAAACATA AAAAATTAAG AGGGGCCAAG ACCAAGACAT TCTTTCTGCA AGGAAATTTC | 4260 |
| TTTTAAATTC TGAACTGCTA CTACACACAA GTGAAAGTCA ACCCTATGTA AACTGCTGTC | 4320 |
| CTCTCTCTAG CCCTCTCCCT TACTGGCCCA CTTCTCTCTC CCTAGAGAGC CTGAAAAACT | 4380 |
| GCCCCAATGC CACGGTAAAG CCCACCAAGT CTTGGCTGGC GTTGCTCACT CACAGTCGCC | 4440 |
| ATCCATCTGG ACACAAAGAG AGACCTGTGG GAGTCATAGA GGGTACTGTT AGCCCCCGTC | 4500 |
| CATGCAGGGG GTTCAGCCGA GCCCAAGACT CAAAGCTGCT TTCCTTTCAG GATTTCTAGT | 4560 |
| AACGTAAGGT CATAATGGCC AAAACTCGTT CTCTCTCATT AAACCAACCA GTAAAACCGT | 4620 |
| ATCCTATTTT TTTGCATAAG GTGTTTCATT TTCCTTTTTA TCGGAAACCA AGGGAAAACC | 4680 |
| ACATTGCGAT CCATTCAGTG TTTAACTGTC GTGGCTCATT TTCTCTTCCT TACCACTTGT | 4740 |
| GTGACAAAAG AGCTCAGATC CGACTTCTCC TATCTCTCAC TTATTCCAAG AACCCAACTA | 4800 |
| TGCCCTTAGG TAGAAAGATT TGACTCGTGT CTCTACTAGC CAACAGGCAG ACCACGGTTG | 4860 |
| AAAAAAATAT CAGCTCCCAA AGGGCCCATG TGTCTACATC ATCAGTTACT GTCATGCACC | 4920 |
| ACATTTGTGT GCACATACCA AAACAGGAGG AAAGAAGAAA AAAATTAATG TGTGGGAGCT | 4980 |
| GCACGTTTAC ATGTTTTCAC CTATGCTTCA AACACAACTG GAAAGCCATC AATCTTCAAA | 5040 |
| GGCCTCAAAA ATACTTTTAT AGTAACAAGT GCACCACTTT AGTTGGGTTA TTCAAGATCG | 5100 |
| CACAAAAACC TTTCCGCAGA GGTGCTATGC TGTGCTTTTG GCGCAAGTGG TGGGGGGATG | 5160 |
| GGGGTGGGGG TGGAATTTTT TTCTCACTCT AATGACTTCC TATTGGAAAG GCATTGACAG | 5220 |
| CCAGGGACAG GAGCCAGGGT GGGGGTAGTT TTGTGGGAAA GCAGAACTGA AGTTAGCTTA | 5280 |
| AGCATAAAAA CAAAGAAAAA TCTTCGCTTT TCATGTATCT GGAATCCAAG AATAACCATA | 5340 |
| GGCTCTACCA GACCAGGAGG GTAAGGATGG ACACTAAAAT GAAACAAATA CCAAGGTATT | 5400 |
| CCTTCTGCTG CAGCCTCCAG ACCACCGAGA GTCGAGCTCC GGCACACACA CACCTGGCCG | 5460 |
| GGACCCGGCA GGGACAAGGC GGGCCGTGGC CTCCTCCACC AAGTCTCTCT AGACAATTCA | 5520 |
| GGGCCTGCTT TCCCCAGCTC CATGCATGGC TGGACTGGTG ATTCCAGGGT GCAGAAGGGA | 5580 |
| TTCATATTCC CAGAACGCTT TAAGTGTACA CCTCCAGCAT AAAGAGATAC CGGTTACATT | 5640 |
| ATTAAATGAT TCTAGGGATT CACTGGGGGA TATTTTTGTT GCTTTTACTT TCATGGTTAG | 5700 |
| AGCTACAAAG AACAGTGATT TTTTTTTTTT CTCCCTTCCC CATTCAGAAA CATTATACAT | 5760 |
| TGCGCCATTT TTCTTTCTCC CAAAGAAGAT TCATGGATAG TCAGACTGAA CTGTGTGCAA | 5820 |
| CAGGAAAAGT CAAAAGGGAA AAGGCAGCTG ATGAGGTTAC ATGGTTACAT GTTCTACATC | 5880 |
| ATGCAGAGTA GCTTGAAATC TAGTCTGGAG AAAACTGGAT CAAGATTCTA GCCCACTGGA | 5940 |
| GTTGCAAGGA ATGAGAGGCA AAAATTCTAA AGATTTGGGT TATATTTTCA ACTTGGGCGA | 6000 |
| CAGAGAGAAA TGGAGAGCAG GAATTACAGT TCCAACAAAC ATCATGATAG TCTGGTAGTC | 6060 |
| AAGACAGAGA TTAAGTAAAA CAGGTTTTAC TGTTTAGCTG AGTTCAGTTA ATACAAAATG | 6120 |
| TACATAAAAC GTTAGTCCTT TGACACTGAC ATGATTAATG ATCAGTGTGG TGGGAAATGA | 6180 |
| TGTAGTTATT GTACACAAGC ACTTGCAAAC TCTTTATCCC TATTTCTTTA AAACAAAATA | 6240 |
| AGGTGAAATA CGAAGTCCTT GGTCTGATAT AAAGCCCCTA TTGGATTCTT CGGATGCGTA | 6300 |
| AAAGAAATTG CCTGTTTCAG CCAGAAGACT GGTGAAAACA CATACATCAG ACTATGTTGT | 6360 |
| GAGCCAGGTT GATTTTTTAT TTTATTATAT GCAGGTGAGT GTTGAAACTG TTAAAATTCC | 6420 |
| AATTTGTTTT CATTCAGTAT TAGTTTAGTT CTAAATATAG CAAACCCCAT CCAGGTGCTA | 6480 |
| TCAGATGACC AGTTACTGCT TAGTTAACTA GGTGTAAAGT TTTACATATA CATTAATTTC | 6540 |
| AATAGTTTAT TACAAGTTGT GTAAAATGGA CTCTAGTTTA ATAATGGGGG AAAAAAGATT | 6600 |
| AGGTTGCTCC TGAAACTGAC TGTAGACCAT GTAAAATGAT TTTACTGGAT TCTGTTCAAC | 6660 |
| TGTAATCAAT GAAAAAGATG TACGTTGTAG ACAAAGTTGC AGAATTAAAA AAACAAATCT | 6720 |
| GCTTTTAATT TATTCTTTTT CTATTAAGAA TTTCTATAGT ATCTTTACAT TTTGCAAAAC | 6780 |
| AGTGTTGTCA ACACTTATTA AAGCATTTTC AAAATG | |

Seq ID NO: 32 Protein sequence:
Protein Accession #: BAA92532

| | |
|---|---|
| 1 11 21 31 41 51 | |
| \| \| \| \| \| \| | |
| MAVQLVPDSA LGLLMMTEGR RCQVHLLDDR KLELLVQPKL LAKELLDLVA SHFNLKEKEY | 60 |
| FGIAFTGETG HLNWLQLDRR VLEHDFPKKS GPVVLYFCVR FYIESISYLK DNATIELFFL | 120 |
| NAKSCIYKEL IDVDSEVVFE LASYILQEAK GDFSSNEVVR SDLKKLPALP TQALKEHPSL | 180 |
| AYCEDRVTEH YKKLNGQTRG QAIVNYMSIV ESLPTYGVHY YAVKDKQGIP WWLGLSYKGH | 240 |
| FQYDYHDKVK PRKIFQWRQL ENLYFREKKF SVEVHDPRRA SVTRRTFGES GIAVHTWYAC | 300 |
| PALIKSIWAM AISQHQFYLD RKQSKSKIHA ARSLSEIAID LTETGTLKTS KLANMGSKGK | 360 |
| IISGSSGSLL SSGSQESDSS QSAKKDMLAA LKSRQEALEE TLRQRLEELK KLCLREAELT | 420 |
| GKLPVEYPLD PGEEPPIVRR RIGTAFKLDE QKILPKGEEA ELERLEREFA IQSQITEAAR | 480 |
| RLASDPNVSK KLKKQRKTSY LNALKKLQEI ENAINENRIK SGKKPTQRAS LIIDDGNIAS | 540 |
| EDSSLSDALV LEDEDSQVTS TISPLESPEK GLPPRPPSHN RPPPPQSLEG LRQMHYHRND | 600 |
| YDKSPIKPKM WSESSLDEPY ERVEKESSES HSSSHKRFPS TGSCAEAGGG SNSLQNSPIR | 660 |
| GLPEWNSQSS MPSTPDLRVR SPHYVHSTRS VDISPTRLHS LALHFRHRSS SLESQGKLLG | 720 |
| SENDTGSPDF YTPRTRSSNG SDPMDDCSSC TSHSSSEHYY PAQMNANYST LAEDSPSKAR | 780 |
| QRQRQRQRAA GALGSASSGS MPNLAARGGA GGAGGAGGGV YLHSQSQPSS QYRIKEYPLY | 840 |
| IEGGATPVVV RSLESDQECH YSVKAQFKTS NSYTAGGLFK ESWRGGGGDE GDTGRLTPSR | 900 |
| SQILRTPSLG REGAHDKGAG RAAVSDELRQ WYQESTASEK EHSRLSHTSS TSSDSGSQYS | 960 |
| TSSQSTFVAH SRVTRMPQMC KATSAALPQS QRSSTPSSEI GATPPSSPHH ILTWQTGEAT | 1020 |
| ENSPILDGSE SPPHQSTDE | |

Seq ID NO: 33 Nucleotide sequence:
Nucleic Acid Accession #: NM_014331
Coding sequence: 1 . . . 1506 (underlined sequences correspond to
start and stop codons)

| | |
|---|---|
| 1 11 21 31 41 51 | |
| \| \| \| \| \| \| | |
| ATGGTCAGAA AGCCTGTTGT GTCCACCATC TCCAAAGGAG GTTACCTGCA GCGAAATGTT | 60 |
| AACGGGAGGC TGCCTTCCCT GGGCAACAAG GAGCCACCTG GGCAGGAGAA AGTGCAGCTG | 120 |
| AAGAGGAAAG TCACTTTACT GAGGGGAGTC TCCATTATCA TTGGCACCAT CATTGGAGCA | 180 |
| GGAATCTTCA TCTCTCCTAA GGGCGTGCTC CAGAACACGG GCAGCGTGGG CATGTCTCTG | 240 |
| ACCATCTGGA CGGTGTGTGG GGTCCTGTCA CTATTTGGAG CTTTGTCTTA TGCTGAATTG | 300 |
| GGAACAACTA TAAAGAAATC TGGAGGTCAT TACACATATA TTTTGGAAGT CTTTGGTCCA | 360 |
| TTACCAGCTT TTGTACGAGT CTGGGTGGAA CTCCTCATAA TACGCCCTGC AGCTACTGCT | 420 |
| GTGATATCCC TGGCATTTGG ACGCTACATT CTGGAACCAT TTTTTATTCA ATGTGAAATC | 480 |
| CCTGAACTTG CGATCAAGCT CATTACAGCT GTGGGCATAA CTGTAGTGAT GGTCCTAAAT | 540 |
| AGCATGAGTG TCAGCTGGAG CGCCCGGATC CAGATTTTCT TAACCTTTTG CAAGCTCACA | 600 |
| GCAATTCTCA TAATTATAGT CCCTGGAGTT ATGCAGCTAA TTAAAGGTCA AACGCAGAAC | 660 |
| TTTAAAGACG CGTTTTCACC AACAGATTCA AGTATTACGC GGTTGCCACT GCCTTTTTAT | 720 |
| TATGGAATGT ATGCATATGC TGGCTGGTTT TACCTCAACT TTGTTACTGA AGAAGTAGAA | 780 |
| AACCCTGAAA AAACCATTCC CCTTGCAATA TGTATATCCA TGGCCATTGT CACCATTGGC | 840 |
| TATGTGCTGA CAAATGTGGC CTACTTTACG ACCATTAATG CTGAGGAGCT GCTGCTTTCA | 900 |
| AATGCAGTCC CACTCACCTT TTCTGAGCGG CTACTCGGAA ATTTCTCATT AGCAGTTCCG | 960 |
| ATCTTTGTTG CCCTCTCCTG CTTTGGCTCC ATGAACGGTG GTGTGTTTGC TGTCTCCAGG | 1020 |
| TTATTCTATG TTGCGTCTCG AGAGGGTCAC CTTCCAGAAA TCCTCTCCAT GATTCATGTC | 1080 |
| CGCAAGCACA CTCCTCTACC AGCTGTTATT GTTTTCCACC CTTTCACAAT GATAATGCTC | 1140 |
| TTCTCTGGAG ACCTCGACAG TCTTTTGAAT TTCCTCAGTT TTGCCAGGTG GCTTTTTATT | 1200 |
| GGGCTGGCAG TTGCTGGGCT GATTTATCTT CCATACAAAT GCCCAGATAT GCATCCTCCT | 1260 |
| TTCAACGTGC CACTCTTCAT CCCACCTTTG TTTTCCTTCA CATCCCTCTT CATCGTTCCC | 1320 |
| CTTTCCCTCT ATTCGGACCC ATTTAGTACA GGGATTGGCT TCGTCATCAC TCTGACTGGA | 1380 |
| GTCCCTGCGT ATTATCTCTT TATTATATGG GACAAGAAAC CCAGGTGGTT TAGAATAATG | 1440 |
| TCAGAGAAAA TAACCAGAAC ATTACAAATA ATACTGGAAG TTGTACCAGA AGAAGATAAG | 1500 |
| TTATGAACTA ATGGACTTGA GATCTTGCCA ATCTGCCCAA GGCCACACAC AAAATACCGA | 1560 |
| TTTTTACTTC ATTTTCTGAA AGTCTAGAGA ATTACAACTT TGGTGATAAA CAAAAGGAGT | 1620 |
| CAGTTATTTT TATTGATATA TTTTAGCATA TTCGAACTAA TTTCTAAGAA ATTTAGTTAT | 1680 |
| AACTCTATGT AGTTATAGAA AGTGAATATG CAGTTATTCT ATGAGTCGCA CAATTCTTGA | 1740 |
| GTCTCTGATA CCTACCTATT GGGGTTAGGA GAAAAGACTA GACAATTACT ATGTGGTGAT | 1800 |
| TCTCTACAAC ATATGTTAGC ACGGCAAAGA ACCTTCAAAT TGAAGACTGA GATTTTTCTG | 1860 |
| TATATATGGG TTTTGTAAAG ATGGTTTTAC ACACTACAGA TGTCTATACT GTGAAAAGTC | 1920 |
| TTTTCAATTC TGAAAAAAAG CATACATCAT GATTATGGCA AAGAGGAGAG AAAGAAATTT | 1980 |
| ATTTTACATT GACATTGCAT TGCTTCCCCT TAGATACCAA TTTAGATAAC AAACACTCAT | 2040 |
| GCTTTAATGG ATTATACCCA GAGCACTTTG AACAAAGGTC AGTGGGGATT GTTGAATACA | 2100 |
| TTAAAGAAGA GTTTCTAGGG GCTACTGTTT ATGAGACACA TCCAGGAGTT ATGTTTAAGT | 2160 |
| AAAAATCCTT GAGAATTTAT TATGTCAGAT GTTTTTTCAT TCATTATCAG GAAGTTTTAC | 2220 |
| TTATCTGTCA TTTTTTTTTT TCACATCAGT TTGATCAGGA AAGTGTATAA CACATCTTAG | 2280 |
| AGCAAGAGTT ACTITGGTAT TAAATCCTCA TTAGAACAAC CACCTGTTTC ACTAATAACT | 2340 |
| TACCCCTGAT GAGTCTATCT AAACATATGC ATTTTAAGCC TTCAAATTAC ATTATCAACA | 2400 |
| TGAGAGAAAT AACCAACAAA GAAGATGTTC AAAATAATAG TCCCATATCT GTAATCATAT | 2460 |
| CTACATGCAA TGTTAGTAAT TCTGAAGTTT TTTAAATTTA TGGCTATTTT TACACGATGA | 2520 |
| TGAATTTTGA CAGTTTGTGC ATTTTCTTTA TACATTTTAT ATTCTTCTGT TAAAATATCT | 2580 |
| CTTCAGATGA AACTGTCCAG ATTAATTAGG AAAAGGCATA TATTAACATA AAAATTGCAA | 2640 |
| AAGAAATGTC GCTGTAAATA AGATTTACAA CTGATGTTTC TAGAAAATTT CCACTTCTAT | 2700 |
| ATCTAGGCTT TGTCAGTAAT TTCCACACCT TAATTATCAT TCAACTTGCA AAAGAGACAA | 2760 |
| CTGATAAGAA GAAAATTGAA ATGAGAATCT GTGGATAAGT GTTTGTGTTC AGAAGATGTT | 2820 |
| GTTTTGCCAG TATTAGAAAA TACTGTGAGC CGGGCATGGT GGCTTACATC TGTAATCCCA | 2880 |
| GCACTTTGGG AGGCTGAGGG GGTGGATCAC CTGAGGTCGG GAGTTCTAGA CCAGCCTGAC | 2940 |
| CAACATGGAG AAACCCCATC TCTACTAAAA ATACAAAATT AGCTGGGCAT GCTGGCACAT | 3000 |
| GCTGGTAATC TCAGCTATTG AGGAGGCTGA GGCAGGAGAA TTGCTTGAAC CCGGGAGGCG | 3060 |
| GAGGTTGCAG TGAGCCAAGA TTGCACCACT GTACTCCAGC CTGGCTCACA AAGTCAGACT | 3120 |
| CCATCTCCAA AAAAAAAAAA AAAA | |

Seq ID NO: 34 Protein sequence:
Protein Accession 4: NP_055146

| | |
|---|---|
| 1 11 21 31 41 51 | |
| \| \| \| \| \| \| | |
| MVRKPVVSTH SKGGYLQGNV NGRLPSLGNK EPPGQEKVQL KRKVTLLRGV SIIIGTIIGA | 60 |
| GIFISPKGVL QNTGSVGMSL TIWTVCGVLS LFGALSYAEL GTTIKKSGGH YTYILEVFGP | 120 |
| LPAFVRVWVE LLIIRPAATA VISLAFGRYI LEPFFIQCEI PELAIKLITA VGITVVMVLN | 180 |
| SMSVSWSART QIFLTFCKLT AILIIIVPGV MQLIKGQTQN FKDAFSGRDS SITRLPLAFY | 240 |
| YGMYAYAGWF YLNFVTEEVE NPEKTIPLAI CISMAIVTIG YVLTNVAYFT TINAEELLLS | 300 |
| NAVAVTFSER LLGNFSLAVP IFVALSCFGS MNGGVFAVSR LFYVASREGH LPEILSMIHV | 360 |
| RKHTPLPAVI VLHPLTMIML FSGDLDSLLN FLSFARWLFI GLAVAGLIYL RYKCPDMHRP | 420 |
| FKVPLFIPAL FSFTCLFMVA LSLYSDPFST GIGFVITLTG VPAYYLFIIW DKKPRWFRIM | 480 |
| SEKITRTLQI ILEVVPEEDK L | |

Seq ID NO: 35 Nucleotide sequence:
Nucleic Acid Accession 4: NM_002422
Coding sequence: 64 . . . 1497 (underlined sequences correspond to
start and stop codons)

| | |
|---|---|
| 1 11 21 31 41 51 | |
| \| \| \| \| \| \| | |
| ACAAGGAGGC ACGCAAGACA GCAAGGCATA GAGACAACAT AGAGCTAAGT AAAGCCAGTG | 60 |
| GAAATGAAGA GTCTTCCAAT CCTACTGTTG CTGTGCGTGG CAGTTTGCTC AGCCTATCCA | 120 |
| TTGGATGGAG CTGCAAGGGG TGAGGACACC AGCATGAACC TTGTTCAGAA ATATCTAGAA | 180 |
| AACTACTACG ACCTCAAAAA AGATGTGAAA CAGTTTGTTA GGAGAAAGGA CAGTGGTCCT | 240 |
| GTTGTTAAAA AAATCCGAGA AATGCAGAAG TTCCTTGGAT TGGAGGTGAC GGGGAAGCTC | 300 |
| GACTCCGACA CTCTCCAGGT GATGCGCAAG CCCAGGTCTC GAGTTCCTGA TGTTGGTCAC | 360 |
| TTCAGAACCT TTCCTGGCAT CCCGAAGTGG AGGAAAACCC ACCTTACATA CAGGATTGTG | 420 |
| AATTATACAC CAGATTTGCC AAAAGATGCT GTTGATTCTG CTGTTGAGAA AGCTCTGAAA | 480 |
| GTCTGGGAAG AGCTCACTCC ACTCACATTC TCCAGGCTGT ATGAACGAGA GGCTGATATA | 540 |
| ATGATCTCTT TTGCAGTTAG AGAACATGGA GACTTTTACC CTTTTGATGG ACCTGGAAAT | 800 |
| GTTTTGCCCC ATGCCTATGC CCCTGGGCCA GGGATTAATG GACATCCCCA CTTTGATGAT | 660 |
| GATGAACAAT GGACAAAGGA TACAACAGGG ACCAATTTAT TTCTCGTTGC TGCTCATCAA | 720 |
| ATTGGCCACT CCCTGGGTCT CTTTCACTCA GCCAACACTG AAGCTTTGAT GTACCCACTC | 780 |
| TATCACTCAC TCACAGACCT GACTCGGTTC CGCCTGTCTC AAGATGATAT AAATGGCATT | 840 |
| CAGTCCCTCT ATGGACCTCC CCCTGACTCC CCTGAGACCC CCCTGGTACC CACGGAACCT | 900 |
| GTCCCTCCAC AACCTGGGAC GCCAGCCAAC TGTGATCCTG CTTTGTCCTT TGATGCTGTC | 960 |
| AGCACTCTGA GGGGAGAAAT CCTGATCTTT AAAGACAGGC ACTTTTGGCC CAAATCCCTC | 1020 |
| AGGAAGCTTG AACCTGAATT GCATTTGATC TCTTCATTTT GGCCATCTCT TCCTTCAGGC | 1080 |
| GTGGATGCCG CATATGAAGT TACTAGCAAG GACCTCGTTT TCATTTTTAA AGGAAATCAA | 1140 |
| TTCTGGGCCA TCAGAGGAAA TGAGGTACGA GCTGGATACC CAAGAGGCAT CCACACCCTA | 1200 |
| GGTTTCCCTC CAACCGTGAG GAAAATCGAT GCAGCCATTT CTGATAACGA AAAGAACAAA | 1260 |
| ACATATTTCT TTGTAGAGGA CAAATACTGG AGATTTGATC AGAAGACAAA TTCCATGGAG | 1320 |
| CCAGGCTTTC CCAAGCAAAT AGCTGAAGAC TTTCCAGGGA TTGACTCAAA GATTGATCCT | 1380 |
| GTTTTTGAAG AATTTGCGTT CTTTTATTTC TTTACTCCAT CTTCACACTT GGAGTTTGAC | 1440 |
| CCAAATGCAA AGAAAGTGAC ACACACTTTG AAGAGTAACA GCTGGCTTAA TTGTTGAAAG | 1500 |
| AGATATGTAG AAGGCACAAT ATGGGCACTT TAAATGAAGC TAATAATTCT TCACCTAAGT | 1560 |
| CTCTGTGAAT TGAAATGTTC GTTTTCTCCT GCCTGTGCTG TGACTCGAGT CACACTCAAG | 1620 |
| GGAACTTGAG CGTGAATCTG TATCTTGCCG GTCATTTTTA TGTTATTACA GGGCATTCAA | 1680 |
| ATGGGCTGCT GCTTAGCTTG CACCTTGTCA CATAGAGTGA TCTTTCCCAA GAGAACGCGA | 1740 |
| AGCACTCGTG TGCAACAGAC AAGTGACTGT ATCTGTGTAG ACTATTTCCT TATTTAATAA | 1800 |
| AGACGATTTG TCAGTTGTTT T | |

Seq ID NO: 36 Protein sequence:
Protein Accession #: NP_002413

| | |
|---|---|
| 1 11 21 31 41 51 | |
| \| \| \| \| \| \| | |
| MKSLPILLLL CVAVCSAYPL DGAARGEDTS MNLVQKYLEN YYDLKKDVKQ FVRRKDSGPV | 60 |
| VKKIREMQKF LGLEVTGKLD SDTLEVMRKP RCGVPDVGHF RTFPGIPKWR KTHLTYRIVN | 120 |
| YTPDLPKDAV DSAVEKALKV WEEVTPLTFS RLYEGEADIM ISFAVREHGD FYPFDGPGNV | 180 |
| LAHAYAPGPG INGDAHFDDD EQWTKDTTGT TNLFLVAAHE GHSLGLFHSA NTEALMYPLY | 240 |
| HSLTDLTRFR LSQDDINGIQ SLYGPPPDSP ETPLVPTEPV PPEPGTPANC DPALSFDAVS | 300 |
| TLRGEILIFK DRHFWRKSLR KLEPELHLIS SFWPSLPSGV DAAYEVTSKD LVFIFKCNQF | 360 |
| WAIRGNEVRA GYPRGIHTLG FPPTVRKIDA AISDKEKNKT YFFVEDKYWR FDEKRNSMEP | 420 |
| GFPKQIASDF PGIDSKIDAV FEEFGFFYFF TGSSQLEFDP NAKKVTHTLK SNSWLNC | |

Seq ID NO: 37 Nucleotide sequence:
Nucleic Acid Accession #: NM_003246
Coding sequence: 112 . . . 3624 (underlined sequences correspond to
start and stop codons)

| | |
|---|---|
| 1 11 21 31 41 51 | |
| \| \| \| \| \| \| | |
| GGACGCACAG GCATTCCCCG CGCCCCTCCA GCCCTCCCCC CCCTCGCCAC CGCTCCCGCC | 60 |
| CGCCGCGCTC CGGTACACAC AGGATCCCTG CTGGGCACCA ACAGCTCCAC CATGGGGCTG | 120 |
| GCCTGGCGAC TAGGCGTCCT GTTCCTGATG CATCTGTGTC GCACCAACCG CATTCCAGAG | 180 |
| TCTGGCGCAG ACAACAGCGT CTTTCACATC TTTGAACTCA CCGGGGCCCC CCGCAAGGGG | 240 |
| TCTGGGCGCC GACTGGTGAA GGGCCCCGAC CCTTCCAGCC CAGCTTTCCG CATCGAGGAT | 300 |
| GCCAACCTGA TCCCCCCTGT GCCTGATGAC AAGTTCCAAG ACCTGGTGGA TGCTGTGCGG | 360 |
| GCAGAAAAGG GTTTCCTCCT TCTGGCATCC CTGAGGCAGA TGAACAAGAC CCGCGGCACG | 420 |
| CTGCTGGCCC TGGAGCGGAA AGACCACTCT GGCCAGGTCT TCAGCGTGGT GTCCAATGGC | 480 |
| AAGGCGGGCA CCCTGGACCT CAGCCTGACC GTCCAAGGAA AGCAGCACGT CGTGTCTGTG | 540 |
| GAAGAAGCTC TCCTGGCAAC CGGCCAGTGC AAGAGCATCA CCCTCTTTGT CCACCAAGAC | 600 |
| AGGGCCCAGC TGTACATCGA CTGTGAAAAG ATGGAGAATG CTGAGTTGGA CGTCCCCATC | 660 |
| CAAAGCGTCT TCACCAGAGA CCTGGCCAGC ATCGCCAGAC TCCGCATCGC AAAGGGGGGC | 720 |
| GTCAATGACA ATTTCCAGGG GGTGCTGCAG AATGTGAGGT TTGTCTTTGG AACCACACCA | 780 |
| GAAGACATCC TCAGCAACAA AGGCTGCTCC ACCTCTACCA GTGTCCTCCT CACCCTTGAC | 840 |
| AACAACGTGG TGAATGGTTC CAGCCCTGCC ATCCGCACTA ACTACATTGG CCACAAGACA | 900 |
| AAGGACTTGC AAGCCATCTG CGGCATCTCC TGTGATGAGC TGTCCAGCAT GGTCCTGGAA | 960 |
| CTCACGGGCC TCCGCACCAT TGTCACCACG CTCCAGGACA GCATCCGCAA AGTGACTGAA | 1020 |
| GAGAACAAAG AGTTGGCCAA TGAGCTGAGG CGGCCTCCCC TATGCTATCA CAACGGAGTT | 1080 |
| CAGTACAGAA ATAACGAGGA ATGGACTGTT CATAGCTGCA CTGAGTGTCA CTGTCAGAAC | 1140 |
| TCAGTTACCA TCTGCAAAAA GGTGTCCTGC CCCATCATGC CCTGCTCCAA TGCCACAGTT | 1200 |
| CCTGATGGAG AATGCTGTCC TCGCTGTTGG CCCAGCGACT CTGCGGACGA TGGCTGGTCT | 1260 |
| CCATGGTCCG AGTGGACCTC CTGTTCTACG AGCTGTGGCA ATGGAATTCA GCAGCGCGGC | 1320 |
| CGCTCCTGCG ATAGCCTCAA CAACCGATGT CAGGGCTCCT CGGTCCAGAC ACGGACCTGC | 1380 |
| CACATTCAGG AGTGTGACAA AAGATTTAAA CAGGATGGTG GCTGGAGCCA CTGGTCCCCG | 1440 |
| TGGTCATCTT GTTCTGTGAC ATGTGCTGAT CGTGTGATCA CAAGGATCCG GCTCTGCAAC | 1500 |
| TCTCCCAGCC CCCAGATGAA TGGGAAACCC TGTGAACGCG AAGCGCGGGA GACCAAACCC | 1560 |
| TGCAAGAAAG ACGCCTGCCC CATCAATGGA GGCTGGGGTC CTTCGTCACC ATGGGACATC | 1620 |
| TGTTCTGTCA CCTGTGGAGG AGGGGTACAG AAACGTAGTC GTCTCTGCAA CAACCCCGCA | 1680 |
| CCCCAGTTTG GAGGCAAGGA CTGCGTTGGT GATGTAACAG AAAACCAGAT CTGCAACAAG | 1740 |
| CAGGACTGTC CAATTGATGG ATCCCTGTCC AATCCCTGCT TTGCCGGCGT GAAGTGTACT | 1800 |
| AGCTACCCTG ATGGCAGCTC GAAATCTGGT GCTTGTCCCC CTGGTTACAG TGGAAATGGC | 1860 |
| ATCCAGTGCA CAGATGTTGA TGAGTGCAAA CAAGTGCCTG ATGCCTGCTT CAACCACAAT | 1920 |
| GGAGAGCACC GCTGTGAGAA CACGCACCCC GGCTACAACT GCCTGCCCTG CCCCCCACGC | 1980 |
| TTCACCCGCT CACAGCCCTT CGGCCAGGGT GTCGAACATG CCACGGCCAA CAAACAGGTG | 2040 |
| TGCAAGCCCC GTAACCCCTG CACGGATGGG ACCCACGACT GCAACAAGAA CGCCAAGTGC | 2100 |
| AACTACCTCG GCCACTATAC CGACCCCATC TACCGCTGCG AGTGCAAGCC TGGCTACCCT | 2160 |
| GCCAATGGCA TCATCTGCGG GGAGGACACA GACCTGGATG GCTGGCCCAA TGAGAACCTG | 2220 |
| GTGTGCCTCC CCAATGCCAC TTACCACTGC AAAAAGGATA ATTGCCCCAA CCTTCCCAAC | 2280 |
| TCAGGGCAGG AAGACTATGA CAAGGATGGA ATTGGTGATG CCTGTGATGA TGACCATGAC | 2340 |
| AATGATAAAA TTCCACATCA CACGCACAAC TGTCCATTCC ATTACAACCC AGCTCAGTAT | 2400 |
| GACTATGACA CACATCATGT CCCACACCGC TGTCACAACT GTCCCTACAA CCACAACCCA | 2460 |
| GATCAGCCAG ACACAGACAA CAATGGGGAA GGAGACGCCT GTGCTGCAGA CATTCATCCA | 2520 |
| CACGGTATCC TCAATGAACG GGACAACTGC CAGTACGTCT ACAATGTGGA CCAGAGAGAC | 2580 |
| ACTGATATCC ATGGGGTTCC ACATCACTGT CACAATTGCC CCTTGGAACA CAATCCGGAT | 2640 |
| CAGCTGGACT CTGACTCAGA CCGCATTCCA CATACCTCTC ACAACAATCA GCATATTGAT | 2700 |
| GAACATCGCC ACCACAACAA TCTCCACAAC TCTCCCTATC TCCCCAATCC CAACCACGCT | 2760 |
| GACCATGACA AAGATCCCAA CCCACATGCC TCTGACCACC ATCATCACAA CGATGGCATT | 2820 |
| CCTCATCACA ACCACAACTC CACACTCCTC CCCAATCCCC ACCACAACCA CTCTGACGCC | 2880 |
| GATGGTCGAG GTGATGCCTG CAAAGATGAT TTTGACCATG ACAGTGTGCC ACACATCCAT | 2940 |
| GACATCTGTC CTGAGAATCT TCACATCACT GACACCGATT TCCCCCCATT CCAGATGATT | 3000 |
| CCTCTGGACC CCAAACCCAC ATCCCAAAAT GACCCTAACT GGGTTGTACG CCATCAGGCT | 3060 |
| AAAGAACTCG TCCAGACTGT CAACTGTGAT CCTGGACTCG CTGTAGGTTA TGATGAGTTT | 3120 |
| AATGCTGTGG ACTTCAGTGG CACCTTCTTC ATCAACACCC AAACGCACCA TGACTATGCT | 3180 |
| GGATTTGTCT TTGGCTACCA CTCCACCACC CCCTTTTATG TTCTGATGTG GAAGCAAGTC | 3240 |
| ACCCAGTCCT ACTGGGACAC CAACCCCACG AGGGCTCAGG GATACTCGGG CCTTTCTGTG | 3300 |
| AAAGTTGTAA ACTCCACCAC AGGGCCTGGC GAGCACCTGC CGAACGCCCT GTGGCACACA | 3360 |
| GGAAACACCC CTGCCCACCT CCGCACCCTG TGGCATGACC CTCCTCACAT AGGCTCCAAA | 3420 |
| GATTTCACCG CCTACAGATG GCGTCTCAGC CACAGGCCAA AGACGGGTTT CATTAGACTG | 3480 |
| GTGATGTATG AAGGGAAGAA AATCATGGCT GACTCAGGAC CCATCTATGA TAAAACCTAT | 3540 |
| GCTGGTGGTA GACTAGGGTT GTTTGTCTTC TCTCAACAAA TCGTGTTCTT CTCTGACCTG | 3600 |
| AAATACGAAT GTAGAGATCC CTAATCATCA AATTGTTGAT TGAAAGACTG ATCATAAACC | 3660 |
| AATGCTGGTA TTGCACCTTC TGGAACTATG GGCTTGAGAA AACCCCCAGG ATCACTTCTC | 3720 |
| CTTGGCTTCC TTCTTTTCTG TGCTTGCATC ACTGTGGACT CCTAGAACGT GCGACCTGCC | 3780 |
| TCAAGAAAAT GCAGTTTTCA AAAACAGACT CATCAGCATT CAGCCTCCAA TGAATAAGAC | 3840 |
| ATCTTCCAAG CATATAAACA ATTGCTTTGC TTTCCTTTTG AAAAACCATC TACTTGCTTC | 3900 |
| AGTTGGGAAG GTGCCCATTC CACTCTGCCT TTGTCACAGA GCACCGTGCT ATTGTGAGGC | 3960 |
| CATCTCTGAG CAGTGGACTC AAAAGCATTT TCAGGCATCT CAGAGAAGGG AGGACTCACT | 4020 |
| AGAATTAGCA AACAAAACCA CCCTCACATC CTCCTTCAGG AACACGGGGA GCAGAGGCCA | 4080 |
| AAGCACTAAG GGGAGGGCGC ATACCCGAGA CGATTGTATG AAGAAAATAT GGAGGAACTG | 4140 |
| TTACATGTTC GGTACTAAGT CATTTTCAGG CGATTGAAAG ACTATTGCTG CATTTCATGA | 4200 |
| TGCTGACTGG CGTTAGCTGA TTAACCCATG TAAATAGGCA CTTAAATAGA AGCAGGAAAG | 4260 |
| GGAGACAAAG ACTGGCTTCT GGACTTCCTC CCTGATCCCC ACCCTTACTC ATCACCTTGC | 4320 |
| AGTGGCCAGA ATTAGGGAAT CAGAATCAAA CCAGTGTAAG GCAGTGCTGG CTGCCATTGC | 4380 |
| CTGGTCACAT TGAAATTGGT GGCTTCATTC TACATGTACC TTCTCCACAT GTACCACGAA | 4440 |
| AATAGGAAAA CCTACCATCT CAGTGAGCAC CAGCTGCCTC CCAAAGGAGG GGCAGCCGTG | 4500 |
| CTTATATTTT TATGGTTACA ATGGCACAAA ATTATTATCA ACCTAACTAA AACATTCCTT | 4560 |
| TTCTCTTTTT TCCGTAATTA CTAGGTAGTT TTCTAATTCT CTCTTTTGGA AGTATGATTT | 4620 |
| TTTTAAAGTC TTTACGATGT AAAATATTTA TTTTTTACTT ATTCTGGAAG ATCTGGCTGA | 4680 |
| AGGATTATTC ATGGAACAGG AAGAAGCGTA AAGACTATCC ATGTCATCTT TGTTGAGACT | 4740 |
| CTTCGTGACT GTAAGATTGT AAATACAGAT TATTTATTAA CTCTGTTCTG CCTGGAAATT | 4800 |
| TAGGCTTCAT ACGGAAAGTG TTTGAGAGCA AGTAGTTGAC ATTTATCAGC AAATCTCTTG | 4860 |
| CAAGAACAGC ACAAGGAAAA TCAGTCTAAT AACCTCCTCT GCCCCTTCTG CTCACACTCC | 4920 |
| ATGTTATGGG ATTCCTTTTT TCTCTGTTTT ATCTTTTCAA GTGGAATTAG TTGGTTATCC | 4980 |
| ATTTGCAAAT GTTTTAAATT GCAAAGAAAG CCATGAGGTC TTCAATACTG TTTTACCCCA | 5040 |
| TCCCTTGTGC ATATTTCCAG CCAGAAGGAA ACCATATACA CTTTTTTCTT TCATTTTTCC | 5100 |
| AAAAGAGAAA AAAATGACAA AAGGTGAAAC TTACATACAA ATATTACCTC ATTTCTTGTG | 5160 |
| TGACTGAGTA AAGAATTTTT GGATCAAGCG GAAAGAGTTT AAGTGTCTAA CAAACTTAAA | 5220 |
| GCTACTGTAG TACCTAAAAA GTCACTGTTG TACATACCAT AAAAACTCTG CAGAGAAGTA | 5280 |
| TTCCCAATAA GCAAATACCA TTGAAATCTT AAATACAATT TCTGAAAGTT ATCTTTTTTT | 5340 |
| TCTATCATCT GGTATACCAT TGCTTTATTT TTATAAATTA TTTTCTCATT GCCATTGGAA | 5400 |
| TAGAATATTC ACATTGTGTA GATATGCTAT TTAAATAATT TATCACGAAA TACTGCCTGT | 5460 |
| AGAGTTAGTA TTTCTATTTT TATATAATGT TTCCACACTC AATTGAAGAA TTGTTGGTTT | 5520 |
| TTTCTTTTTT TTGTTTTTTT TTTTTTTTTT TTTTTTTTTG CTTTTGACCT CCCATTTTTA | 5580 |
| CTATTTGCCA ATACCTTTTT CTACGAATGT CCTTTTTTTT GTACACATTT TTATCCATTT | 5640 |
| TACATTCTAA AGCAGTGTAA GTTGTATATT ACTGTTTCTT ATGTACAAGG AACAACAATA | 5700 |
| AATCATATGG AAATTTATAT TT | |

Seq ID NO: 38 Protein sequence:
Protein Accession #: NP_003237

| | |
|---|---|
| 1 11 21 31 41 51 | |
| \| \| \| \| \| \| | |
| MGLAWGLGVL FLMHVCGTNR IPESGGDNSV PDIFELTGAA RKGSGRRLVK GPDPSSPAFR | 60 |
| IEDANLIPPV PDDKFQDLVD AVRAEKGFLL LASLRQMKKT RGTLLALERK DHSGQVFSVV | 120 |
| SNGKAGTLDL SLTVQGKQHV VSVEEALLAT GQWKSITLFV QEDRAQLYID CEKMENAELD | 180 |
| VPIQSVFTRD LASIARLRIA KGGVNDNFQG VLQNVRFVEG TTPEDILRNK CCSSSTSVLL | 240 |
| TLDNNVVWGS SPAIRTNYIG HKTKDLQAIC GISCDELSSM VLELRGLRTI VTTLQDSIRK | 300 |
| VTEENKELAN ELRRPPLCYH NGVQYRNNEE WTVDSCTECH CQNSVTICKK VSCPIMPCSN | 360 |
| ATVPDGECCP RCWPSDSADD GWSPWSEWTS CSTSCGNGIQ QRGRSCDSLN NRCEGSSVQT | 420 |
| RTCHIQECDK RFKQDGGWSH WSPWSSCSVT CGDGVITRIR LCNSPSPQMN GKPCEGEARE | 480 |
| TKACKKDACP INGGWGPWSP WDICSVTCGG GVQKRSRLCN NPAPQFGGKD CVGDVTENQI | 540 |
| CNKQDCPIDG CLSNPCFAGV KCTSYPDGSW KCGACPPGYS GNGIQCTDVD ECKEVPDACF | 600 |
| NHNGEHRCEN TDPGYNCLPC PPRFTGSQPF GQGVEHATAN KQVCKPRNPC TDGTHDCNKN | 660 |
| AKCNYLGHYS DPMYRCECKP GYAGNGIICG EDTDLDGWPN ENLVCVANAT YHCKKDNCPN | 720 |
| LPNSGQEDYD KDGIGDACDD DDDNDKIPDD RDNCPFHYNP AQYDYDRDDV GDRCDNCPYN | 780 |
| HNPDQADTDN NGEGDACAAD IDGDGILNER DNCQYVYNVD QRDTDMDGVC DQCDNCPLEH | 840 |
| NPDQLDSDSD RIGDTCDNNQ DIDEDGHQNN LDNCPYVPNA NQADHDKDGK GDACDHDDDN | 900 |
| DGIPDDKDNC RLVPNPDQKD SDGDGRGDAC KDDFDHDSVP DIDDICPENV DISETDFRRF | 960 |
| QMIPLDPKGT SQNDPNWVVR HQGKELVQTV NCDPGLAVGY DEFNAVDFSG TFFINTERDD | 1020 |
| DYAGFVFGYQ SSSRFYVVMW KQVTQSYWDT NPTRAQGYSG LSVKVVNSTT GPGEHLRNAL | 1080 |
| WHTGNTPGQV RTLWHDPRHI GWKDFTAYRW RLSHRPKTGF IRVVNYEGKK IMADSGPIYD | 1140 |
| KTYAGGRLGL FVFSQEMVFF SDLKYECRDP | |

Seq ID NO: 39 Nucleotide sequence:
Nucleic Acid Accession #: BC004299
Coding sequence: 69 . . . 1235 (underlined sequences correspond to
start and stop codons)

| | |
|---|---|
| 1 11 21 31 41 51 | |
| \| \| \| \| \| \| | |
| CCCGACCCGT GCGAGGGCCA GGTCCGCCCC TCCCCCGCCA GGCGAAGCGA GGCGACCCGC | 60 |
| GTGCGGCCAT CCCTTCCCTG CTGGGAGCCT ACCCTTGGCC CGAGGGTCTC GAGTGCCCCC | 120 |
| CCCTGGACGC CGAGCTGTCG GATGGACAAT CGCCGCCCGC CGTCCCCCGG CCCCCGGGGG | 180 |
| ACAAGGGCTC CGAGAGCCGT ATCCGGCGGC CCATGAACGC CTTCATGGTT TGGGCCAAGG | 240 |
| ACGAGAGGAA ACGCCTCCCA GTGCACAACC CGGACCTGCA CAACGCCGAG CTCAGCAAGA | 300 |
| TGCTGGGAAA GTCGTGGAAG GCGCTGACGC TGTCCCAGAA GAGGCCGTAC GTGGACGAGG | 360 |
| CGGAGCGGCT GCGCCTGCAG CACATGCAGG ACTACCCCAA CTACAAGTAC CGGCCGCGCA | 420 |
| GGAAGAAGCA GGCCAAGCGG CTGTGCAAGC GCGTGGACCC GGGCTTCCTT CTGAGCTCCC | 480 |
| TCTCCCGGGA CCAGAACGCC CTGCCGGAGA AGAGAAGCGG CAGCCGGGGG GCGCTGGGGG | 540 |
| AGAAGGAGGA CAGGGGTGAG TACTCCCCCG GCACTGCCCT GCCCAGCCTC CGGGGCTGCT | 600 |
| ACCACGAGGG GCCGGCTGGT GGTGGCGGCG GCGGCACCCC GAGCAGTGTG GACACGTACC | 660 |
| CGTACGGGCT GCCCACACCT CCTGAAATGT CTCCCCTGGA CGTGCTGGAG CCGGAGCAGA | 720 |
| CCTTCTTCTC CTCCCCCTGC CAGGAGGAGC ATGGCCATCC CCGCCGCATC CCCCACCTGC | 780 |
| CAGGGCACCC GTACTCACCG GAGTACGCCC CAAGCCCTCT CCACTGTAGC CACCCCCTGG | 840 |
| GCTCCCTGGC CCTTGGCCAG TCCCCCGGCG TCTCCATGAT GTCCCCTGTA CCCGGCTGTC | 900 |
| CCCCATCTCC TGCCTATTAC TCCCCGGCCA CCTACCACCC ACTCCACTCC AACCTCCAAG | 960 |
| CCCACCTGGG CCAGCTTTCC CCGCCTCCTG AGCACCCTGG CTTCGACGCC CTGGATCAAC | 1020 |
| TGAGCCAGGT GGAACTCCTG GGGGACATGG ATCGCAATGA ATTCGACCAG TATTTGAACA | 1080 |
| CTCCTGGCCA CCCAGACTCC GCCACAGGGG CCATGGCCCT CAGTGGGCAT GTTCCGGTCT | 1140 |
| CCCAGGTGAC ACCAACGGGT CCCACAGAGA CCAGCCTCAT CTCCGTCCTG GCTGATGCCA | 1200 |
| CGGCCACGTA CTACAACAGC TACAGTGTGT CATAGAGCTG GAGGCGCCCC GTCCGGTCAG | 1260 |
| CCCTCGCGCC CTCTCCTTCT TGTGCCTTGA GTGGCAGAGG AGCCGTCCAG CCACACCAGC | 1320 |
| TTTCCTCCCA CCGCTCAGGG CAGGGAGGTC TGAACTGCGG CCCCAGAGCC TTTGGCCTAA | 1380 |
| GCTGGACTCT CCTTATCCGA GTGCCGCCTC TATCCCCTTC CCCACGTTCC AGCCCCTGCA | 1440 |
| GCCCACATTT TAAGTATATT CCTTCAAGTG AGTTTTCCTC CAGCCCCTGA GAGTTGCTGT | 1500 |
| CTCCCAGTGG AATGTTCACT GACGTCTTTT CTTGGTAGCC ATCATCGAAA CTAATGGGGG | 1560 |
| GACAGACTTG ATAGCCAAGG TCCCTTCTGG TCCAGTTTTC TGATTTAGGG TTCTCTCAAG | 1620 |
| ATTAATAAAG GAAGATGGGG AAATTTGACT CATTAATGAG CTCGCTAACC TACGATCTGG | 1680 |
| TGATAATTTT GTGTGCACAG CCCAAGGACC ACGAGGCTTT CTGCACTTTC TGCACCCCCT | 1740 |
| TCCAAAGTGA CCACAAAATT TCAAAGGGAC TCATACAATT TGAGAAAAAA CAGTCAACCT | 1800 |
| GATTTGAGAA ATTAACCAGT ATGGCTAACT ATATCACAGA AAATGGGATT GAGTTAAAAC | 1860 |
| TATTTTATTT TAAATATACA TTTTAAAGCA GTTCTTTTTT TTTGTTAATT TGTTTATTAT | 1920 |
| ACACACACTT CAAGAGCCAC CGCGCCCAGC CTACATTTAT AATTTTCATT CTCTTTTACC | 1980 |
| TATAAAATTC AGTGTATTAG TTTCATTACA TAGGAGAAAT TATATTTCTA AACATTTTAT | 2040 |
| GATGTTTAAA AACAAAACAG GCTGTTGTAA AAAAAAAAAA AAAAAAAAA | |

Seq ID NO: 40 Protein sequence:
Protein Accession #: AAH04299

| | |
|---|---|
| 1 11 21 31 41 51 | |
| \| \| \| \| \| \| | |
| MASLLGAYPW PEGLECPALD AELSDGQSPP AVPRPPGDKG SESRIRRPMN AFMVWAKDER | 60 |
| KRLAVQNPDL HNAELSKMLG KSWKALTLSQ KRPYVDEAER LRLQHMQDYP NYKYRPRRKK | 120 |
| QAKRLCKRVD PGFLLSSLSR DQNALPEKRS GSRGALGEKE DRGEYSPGTA LPSLRGCYHE | 180 |
| GPAGGGGGGT PSSVDTYPYG LPTPPEMSPL DVLEPEQTFF SSPCQEEHGH PRRIPHLPGH | 240 |
| PYSPEYAPSP LHCSHPLGSL ALGQSPGVSM MSPVPGCPPS PAYYSPATYH PLHSNLQAHL | 300 |
| GQLSPPPEHP GFDALDQLSQ VELLGDMDRN EFDQYLNTPG HPDSATGAMA LSGHVPVSQV | 360 |
| TPTGPTETSL ISVLADATAT YYNSYSVS | |

Seq ID NO: 41 Nucleotide sequence:
Nucleic Acid Accession #: NM_004449
Coding sequence: 1 . . . 1389 (underlined sequences correspond to
start and stop codons)

| | |
|---|---|
| 1 11 21 31 41 51 | |
| \| \| \| \| \| \| | |
| ATGATTCAGA CTGTCCCGGA CCCAGCAGCT CATATCAAGG AAGCCTTATC AGTTGTGAGT | 60 |
| GAGGACCAGT CGTTGTTTGA GTGTGCCTAC GGAACGCCAC ACCTGGCTAA GACAGAGATG | 120 |
| ACCGCGTCCT CCTCCAGCGA CTATGGACAG ACTTCCAAGA TGAGCCCACG CGTCCCTCAG | 180 |
| CAGGATTGGC TGTCTCAACC CCCAGCCAGG GTCACCATCA AAATGGAATG TAACCCTAGC | 240 |
| CAGGTGAATG GCTCAAGGAA CTCTCCTGAT GAATGCAGTG TGGCCAAAGG CGGGAAGATG | 300 |
| GTGGGCAGCC CAGACACCGT TGGGATGAAC TACGGCAGCT ACATGGAGGA GAAGCACATG | 360 |
| CCACCCCCAA ACATGACCAC GAACGAGCGC AGAGTTATCG TGCCAGCAGA TCCTACGCTA | 420 |
| TGGAGTACAG ACCATGTGCG GCAGTGGCTG GAGTGGGCGG TGAAAGAATA TGGCCTTCCA | 480 |
| GACGTCAACA TCTTGTTATT CCAGAACATC GATGGGAAGG AACTGTGCAA GATGACCAAG | 540 |
| GACGACTTCC AGAGGCTCAC CCCCAGCTAC AACGCCGACA TCCTTCTCTC ACATCTCCAC | 600 |
| TACCTCAGAG AGACTCCTCT TCCACATTTG ACTTCAGATG ATGTTGATAA AGCCTTACAA | 660 |
| AACTCTCCAC GGTTAATGCA TGCTAGAAAC ACAGATTTAC CATATGAGCC CCCCAGGAGA | 720 |
| TCAGCCTGGA CCGGTCACGG CCACCCCACG CCCCAGTCGA AAGCTGCTCA ACCATCTCCT | 780 |
| TCCACAGTGC CCAAAACTGA AGACCAGCGT CCTCAGTTAG ATCCTTATCA GATTCTTGGA | 840 |
| CCAACAAGTA GCCGCCTTGC AAATCCAGGC AGTGGCCAGA TCCAGCTTTG GCAGTTCCTC | 900 |
| CTGGAGCTCC TGTCGGACAG CTCCAACTCC AGCTGCATCA CCTGGGAAGG CACCAACGGG | 960 |
| GAGTTCAAGA TGACGGATCC CGACGAGGTG GCCCGGCGCT GGGGAGAGCG GAAGAGCAAA | 1020 |
| CCCAACATGA ACTACGATAA GCTCAGCCGC GCCCTCCGTT ACTACTATGA CAAGAACATC | 1080 |
| ATGACCAAGG TCCATGGGAA GCGCTACGCC TACAAGTTCG ACTTCCACGG GATCGCCCAG | 1140 |
| GCCCTCCAGC CCCACCCCCC GGAGTCATCT CTGTACAAGT ACCCCTCAGA CCTCCCGTAC | 1200 |
| ATGGGCTCCT ATCACGCCCA CCCACAGAAG ATGAACTTTG TGGCGCCCCA CCCTCCAGCC | 1260 |
| CTCCCCGTGA CATCTTCCAG TTTTTTTGCT GCCCCAAACC CATACTGGAA TTCACCAACT | 1320 |
| GGGGGTATAT ACCCCAACAC TAGGCTCCCC ACCAGCCATA TGCCTTCTCA TCTGGGCACT | 1380 |
| TACTACTAA | |

Seq ID NO: 42 Protein sequence:
Protein Accession #: NP_004440

| | |
|---|---|
| 1 11 21 31 41 51 | |
| \| \| \| \| \| \| | |
| MIQTVPDPAA HIKEALSVVS EDQSLFECAY GTPHLAKTEM TASSSSDYGQ TSKMSPRVPQ | 60 |
| QDWLSQPPAR VTIKMECNPS QVNGSRNSPD ECSVAKGGKM VGSPDTVGMN YGSYMEEKHM | 120 |
| PPPNMTTNER RVIVPADPTL WSTDHVRQWL EWAVKEYGLP DVNILLFQNI DCKELCKMTK | 180 |
| DDFQRLTPSY NADILLSHLH YLRETPLPHL TSDDVDKALQ NSPRLMHARN TDLPYEPPRR | 240 |
| SAWTGHGHPT PQSKAAQPSP STVPKTEDQR PQLDPYQILG PTSSRLANPG SGQIQLWQFL | 300 |
| LELLSDSSNS SCITWEGTNG EFKNTDPDEV ARRWGERKSK PNMNYDKLSR ALRYYYDKNI | 360 |
| MTKVHGKRYA YKFDFNGIAQ ALQPHPPESS LYKYPSDLPY MGSYHAHPGK MNFVAPHPPA | 420 |
| LPVTSSSFFA APNPYWNSPT GGIYPNTRLP TSHMPSHLGT YY | |

Seq ID NO: 43 Nucleotide sequence:
Nucleic Acid Accession #: NM_005100
Coding sequence: 192 . . . 5537 (underlined sequences correspond to
start and stop codons)

| | |
|---|---|
| 1 11 21 31 41 51 | |
| \| \| \| \| \| \| | |
| CCTTCTTTTA AGGAGTTTGC CGCGAGCGCG TCTCCTTCAT TCGCAGGCTG GGCGCGTTCG | 60 |
| CAGTCGGCTG GCGGCGAAGG AAGGCGCTCT CGGGACCTCA CGGGCGCGCG TCTTTTGGCT | 120 |
| CTTGCCCCTG TCCCTGCGGC TTGGGGAAAG CGTAACCCGG CGGCTAGGCG CGGGAGAAGT | 180 |
| GCGGAGGAGC CATGGGCGCC GGGAGCTCCA CCGAGCAGCG CAGCCCGGA CAGCCGCCCG | 240 |
| AGGGGAGCTC CACGCCGGCT GAGCCCGAGC CCAGCGGCGG CGGCCCCTCG GCCGAGGCGG | 300 |
| CGCCAGACAC CACCGCGGAC CCCGCCATCG CTGCCTCGGA CCCCGCCACC AAGCTCCTAC | 360 |
| AGAAGAATGG TCAGCTGTCC ACCATCAATG GCGTAGCTGA GCAAGATGAG CTCAGCCTCC | 420 |
| AGGAGGGTGA CCTAAATGGC CAGAAAGGAG CCCTGAACGG TCAAGGAGCC CTAAACAGCC | 480 |
| AGGAGGAAGA AGAAGTCATT GTCACGGAGG TTGGACAGAG AGACTCTGAA GATGTGAGCG | 540 |
| AAAGAGACTC CGATAAAGAG ATGGCTACTA AGTCAGCGGT TGTTCACGAC ATCACAGATG | 600 |
| ATGGGCAGGA GGAGAACCGA AATATCGAAC AGATTCCTTC TTCAGAAAGC AATTTAGAAG | 660 |
| AGCTAACACA ACCCACTGAG TCCCAGGCTA ATGATATTGG ATTTAAGAAG GTGTTTAAGT | 720 |
| TTGTTGGCTT TAAATTCACT GTGAAAAAGG ATAAGACAGA GAAGCCTGAC ACTGTCCAGC | 780 |
| TACTCACTGT GAAGAAAGAT GAAGGGGAGG GAGCAGCAGG GGCTGGCGAC CACCAGGACC | 840 |
| CCAGCCTTGG GGCTGGAGAA GCAGCATCCA AAGAAAGCGA ACCCAAACAA TCTACAGAGA | 900 |
| AACCCGAAGA GACCCTGAAG CGTGAGCAAA GCCACGCAGA AATTTCTCCC CCAGCCGAAT | 960 |
| CTGGCCAAGC AGTGGAGGAA TGCAAAGAGG AAGGAGAAGA GAAACAAGAA AAAGAACCTA | 1020 |
| GCAAGTCTGC AGAATCTCCG ACTAGTCCCG TGACCAGTGA AACAGGATCA ACCTTCAAAA | 1080 |
| AATTCTTCAC TCAAGGTTGG GCCGGCTGGC GCAAAAAGAC CAGTTTCAGG AAGCCGAAGG | 1140 |
| AGGATGAAGT GGAAGCTTCA GAGAAGAAAA AGGAACAAGA GCCAGAAAAA GTAGACACAG | 1200 |
| AAGAAGACGG AAAGGCAGAG GTTGCCTCCG AGAAACTGAC CGCCTCCGAG CAAGCCCACC | 1260 |
| CACAGGAGCC GGCAGAAAGT GCCCACGAGC CCCGGTTATC AGCTGAATAT GAGAAAGTTG | 1320 |
| AGCTGCCCTC AGAGGAGCAA GTCAGTGGCT CGCAGGGACC TTCTGAAGAG AAACCTGCTC | 1380 |
| CGTTGGCGAC AGAAGTGTTT GATGAGAAAA TAGAAGTCCA CCAAGAAGAG GTTGTGGCCG | 1440 |
| AAGTCCACGT CAGCACCGTG GAGGAGAGAA CCGAAGAGCA GAAAACGGAG GTGGAAGAAA | 1500 |
| CAGCAGGGTC TGTGCCAGCT GAAGAATTGG TTGGAATGGA TGCAGAACCT CAGGAAGCCG | 1560 |
| AACCTGCCAA GGAGCTGGTG AAGCTCAAAG AAACGTGTGT TTCCGGAGAG GACCCTACAC | 1620 |
| AGGGAGCTGA CCTCAGTCCT GATGAGAAGG TGCTGTCCAA ACCCCCCGAA GGCGTTGTGA | 1680 |
| GTGAGGTGGA AATGCTGTCA TCACAGGAGA GAATGAAGGT GCAGGGAAGT CCACTAAAGA | 1740 |
| AGCTTTTTAC CAGCACTGGC TTAAAAAAGC TTTCTGGAAA GAAACAGAAA GGGAAAAGAG | 1800 |
| GAGGAGGAGA CGAGGAATCA GGGGAGCACA CTCAGGTTCC AGCCGATTCT CCGGACAGCC | 1860 |
| AGGAGGAGCA AAAGGGCGAG AGCTCTGCCT CATCCCCTGA GGAGCCCGAG GAGATCACGT | 1920 |
| GTCTGGAAAA GGGCTTAGCC GAGGTGCAGC AGGATGGGGA AGCTGAAGAA GGAGCTACTT | 1980 |
| CCGATGGAGA GAAAAAAAGA GAAGGTGTCA CTCCCTGGGC ATCATTCAAA AAGATGGTGA | 2040 |
| CGCCCAAGAA GCGTGTTAGA CGGCCTTCGG AAAGTGATAA AGAAGATGAG CTGGACAAGG | 2100 |
| TCAAGAGCGC TACCTTGTCT TCCACCGAGA GCACAGCCTC TGAAATGCAA GAAGAAATGA | 2160 |
| AAGGGAGCGT GGAAGAGCCA AAGCCGGAAG AACCAAAGCG CAAGGTGGAT ACCTCAGTAT | 2220 |
| CTTGGGAAGC TTTAATTTGT GTGGGATCAT CCAAGAAAAG AGCAAGGAGA AGGTCCTCTT | 2280 |
| CTGATGAGGA AGGGGGACCA AAAGCAATGG GAGGAGACCA CCAGAAAGCT GATGAGGCCG | 2340 |
| GAAAAGACAA AGAGACGGGG ACAGACGGGA TCCTTGCTGG TTCCCAAGAA CATGATCCAG | 2400 |
| GGCAGGGAAG TTCCTCCCCG GAGCAAGCTG GAAGCCCTAC CGAAGGGGAG GGCGTTTCCA | 2460 |
| CCTGGGAGTC ATTTAAAAGG TTAGTCACGC CAAGAAAAAA ATCAAAGTCC AAGCTGGAAG | 2520 |
| AGAAAAGCGA AGACTCCATA GCTGGGTCTG GTGTAGAACA TTCCACTCCA GACACTGAAC | 2580 |
| CCGGTAAAGA AGAATCCTGG GTCTCAATCA AGAAGTTTAT TCCTGGACGA AGGAAGAAAA | 2640 |
| GGCCAGATGG GAAACAAGAA CAAGCCCCTG TTGAAGACGC AGGGCCAACA GGGGCCAACG | 2700 |
| AAGATGACTC TGATGTCCCG GCCGTGGTCC CTCTGTCTGA GTATGATGCT GTAGAAAGGG | 2760 |
| AGAAAATGGA GGCACAGCAA GCCCAAAAAG GCGCAGAGCA GCCCGAGCAG AAGGCAGCCA | 2820 |
| CTGAGGTGTC CAAGGAGCTC AGCGAGAGTC AGGTTCATAT GATGGCAGCA GCTGTCGCTG | 2880 |
| ACGGGACGAG GGCAGCTACC ATTATTGAAG AAAGGTCTCC TTCTTGGATA TCTGCTTCAG | 2940 |
| TGACAGAACC TCTTGAACAA GTAGAAGCTG AAGCCGCACT GTTAACTGAG GAGGTATTGG | 3000 |
| AAAGAGAAGT AATTGCAGAA GAAGAACCCC CCACGGTTAC TGAACCTCTG CCAGAGAACA | 3060 |
| GAGAGGCCCG GGGCGACACG GTCGTTAGTG AGGCGGAATT GACCCCCGAA GCTGTGACAG | 3120 |
| CTGCAGAAAC TGCAGGGCCA TTGGGTTCCG AAGAAGGAAC CGAAGCATCT GCTGCTGAAG | 3180 |
| AGACCACAGA AATGGTGTCA GCAGTCTCCC AGTTAACCGA CTCCCCAGAC ACCACAGAGG | 3240 |
| AGGCCACTCC GGTGCAGGAG GTGGAAGGTG GCGTACCTGA CATAGAAGAG CAAGAGAGGC | 3300 |
| GGACTCAAGA GGTCCTCCAG GCAGTGGCAG AAAAAGTGAA AGAGGAATCC CAGCTGCCTG | 3360 |
| GCACCGGTGG GCCAGAAGAT GTGCTTCAGC CTGTGCAGAG AGCAGAGGCA GAAAGACCAG | 3420 |
| AAGAGCAGGC TGAAGCGTCG GGTCTGAAGA AAGAGACGGA TGTAGTGTTG AAAGTAGATG | 3480 |
| CTCAGGAGGC AAAAACTGAG CCTTTTACAC AAGGGAAGGT GGTGGGGCAG ACCACCCCAG | 3540 |
| AAAGCTTTGA AAAAGCTCCT CAAGTCACAG AGAGCATAGA GTCCAGTGAG CTTGTAACCA | 3600 |
| CTTGTCAAGC CGAAACCTTA GCTGGGGTAA AATCACAGGA GATGGTGATG GAACAGGCTA | 3660 |
| TCCCCCCTGA CTCGGTGGAA ACCCCTACAG ACAGTGAGAC TGATGGAAGC ACCCCCGTAG | 3720 |
| CCGACTTTGA CGCACCAGGC ACAACCCAGA AAGACGAGAT TGTGGAAATC CATGAGGAGA | 3780 |
| ATGAGGTCGC ATCTGGTACC CAGTCAGGGG GCACAGAAGC AGAGGCAGTT CCTGCACAGA | 3840 |
| AAGAGAGGCC TCCAGCACCT TCCAGTTTTG TGTTCCAGGA AGAAACTAAA GAACAATCAA | 3900 |
| AGATGGAAGA CACTCTAGAG CATACAGATA AAGAGGTGTC AGTGGAAACT GTATCCATTC | 3960 |
| TGTCAAAGAC TGAGGGGACT CAAGAGGCTG ACCAGTATGC TGATGAGAAA ACCAAAGACG | 4020 |
| TACCATTTTT CGAAGGACTT GAGGGGTCTA TAGACACAGG CATAACAGTC AGTCGGGAAA | 4080 |
| AGGTCACTGA AGTTGCCCTT AAAGGTGAAG GGACAGAAGA AGCTGAATGT AAAAAGGATG | 4140 |
| ATGCTCTTGA ACTGCAGAGT CACGCTAAGT CTCCTCCATC CCCCGTGGAG AGAGAGATGG | 4200 |
| TAGTTCAAGT CGAAAGGGAG AAAACAGAAG CAGAGCCAAC CCATGTGAAT GAAGAGAAGC | 4260 |
| TTGAGCACGA AACAGCTGTT ACCGTATCTG AAGAGGTCAG TAAGCAGCTC CTCCAGACAG | 4320 |
| TGAATGTGCC CATCATAGAT GGGGCAAAGG AAGTCAGCAG TTTGGAAGGA AGCCCTCCTC | 4380 |
| CCTGCCTAGG TCAAGAGGAG GCAGTATGCA CCAAAATTCA AGTTCAGAGC TCTGAGGCAT | 4440 |
| CATTCACTCT AACAGCGGCT GCAGAGGAGG AAAAGGTCTT AGGAGAAACT GCCAACATTT | 4500 |
| TAGAAACAGG TGAAACGTTG GAGCCTGCAG GTGCACATTT AGTTCTGGAA GAGAAATCCT | 4560 |
| CTGAAAAAAA TGAAGACTTT GCCGCTCATC CAGGGGAAGA TGCTGTGCCC ACAGGGCCCG | 4620 |
| ACTGTCAGGC AAAATCGACA CCAGTGATAG TATCTGCTAC TACCAAGAAA GGCTTAAGTT | 4680 |
| CCGACCTGGA AGGAGAGAAA ACCACATCAC TGAAGTGGAA GTCAGATGAA GTCGATGAGC | 4740 |
| AGGTTGCTTG CCAGGAGGTC AAAGTGAGTG TAGCAATTGA GGATTTAGAG CCTGAAAATG | 4800 |
| GGATTTTGGA ACTTGAGACC AAAAGCAGTA AACTTGTCCA AAACATCATC CAGACAGCCG | 4860 |
| TTGACCAGTT TGTACGTACA GAAGAAACAG CCACCGAAAT GTTGACGTCT GAGTTACAGA | 4920 |
| CACAAGCTCA CGTGATAAAA GCTGACAGCC AGGACGCTGG ACAGGAAACG GAGAAAGAAG | 4980 |
| GAGAGGAACC TCAGGCCTCT GCACAGGATG AAACACCAAT TACTTCAGCC AAAGAGGAGT | 5040 |
| CAGAGTCAAC CGCAGTGGGA CAAGCACATT CTGATATTTC CAAAGACATG AGTGAAGCCT | 5100 |
| CAGAAAAGAC CATGACTGTT GAGGTAGAAG GTTCCACTGT AAATGATCAG CAGCTGGAAG | 5160 |
| AGGTCGTCCT CCCATCTGAG GAAGAGGGAG GTGGAGCTGG AACAAAGTCT GTGCCAGAAG | 5220 |
| ATGATGGTCA TGCCTTGTTA GCAGAAAGAA TAGAGAAGTC ACTAGTTGAA CCGAAAGAAG | 5280 |
| ATGAAAAAGG TGATGATGTT GATGACCCTG AAAACCAGAA CTCAGCCCTG GCTGATACTG | 5340 |
| ATGCCTCAGG AGGCTTAACC AAAGAGTCCC CAGATACAAA TGGACCAAAA CAAAAAGAGA | 5400 |
| AGGAGGATGC CCAGGAAGTA GAATTGCAGG AAGGAAAAGT GCACAGTGAA TCAGATAAAG | 5460 |
| CGATCACACC CCAAGCACAG GAGGAGTTAC AGAAACAAGA GAGAGAATCT GCAAAGTCAG | 5520 |
| AACTTACAGA ATCTTAAAAC ATCATGCAGT TAAACTCATT GTCTGTTTGG AAGACCAGAA | 5580 |
| TGTGAAGACA AGTAGTAGAA GAAAATGAAT GCTGCTGCTG AGACTGAAGA CCAGTATTTC | 5640 |
| AGAACTTTGA GAATTGGAGA GCAGGCACAT CAACTGATCT CATTTCTAGA GAGCCCCTGA | 5700 |
| CAATCCTGAG GCTTCATCAG GAGCTAGAGC CATTTAACAT TTCCTCTTTC CAAGACCAAC | 5760 |
| CTACAATTTT CCCTTGATAA CCATATAAAT TCTGATTTAA GGTCCTAAAT TCTTAACCTG | 5820 |
| GAACTGGAGT TGGCAATACC TAGTTCTGCT TCTGAAACTG GACTATCATT CTTTACATAT | 5880 |
| TTATATGTAT GTTTTAAGTA GTCCTCCTGT ATCTATTGTA TATTTTTTTC TTAATGTTTA | 5940 |
| AGGAAATGTG CAGGATACTA CATGCTTTTT GTATCACACA GTATATGATG GGGCATGTGC | 6000 |
| CATAGTGCAG GCTTGGGGAG CTTTAAGCCT CAGTTATATA ACCCACAAAA AACAGAGCCT | 6060 |
| CCTAGATGTA ACATTCCTGA TCAAGGTACA ATTCTTTAAA ATTCACTAAT GATTGAGGTC | 6120 |
| CATATTTAGT GGTACTCTGA AATTGGTCAC TTTCCTATTA CACGGAGTGT GCCAAAACTA | 6180 |
| AAAAGCATTT TGAAACATAC AGAATGTTCT ATTGTCATTG CGAAATTTTG CTTTCTAACC | 6240 |
| CAGTGGAGGT TAGAAAGAAG TTATATTCTG GTAGCAAATT AACTTTACAT CCTTTTTCCT | 6300 |
| ACTTGTTATG GTTGTTTGGA CCGATAAGTG TGCTTAATCC TGAGGCAAAG TAGTGAATAT | 6360 |
| GTTTTATATG TTATGAAGAA AAGAATTGTT GTAAGTTTTT GATTCTACTC TTATATGCTG | 6420 |
| GACTGCATTC ACACATGGCA TGAAATAAGT CAGGTTCTTT ACAAATGGTA TTTTGATAGA | 6480 |
| TACTGGATTG TGTTTGTGCC ATATTTGTGC CATTCCTTTA AGAACAATGT TGCAACACAT | 6540 |
| TCATTTGGAT AAGTTGTGAT TTGACGACTG ATTTAAATAA AATATTTGCT TCACTTAAAA | 6600 |
| AAAAAAAA | |

Seq ID NO: 44 Protein sequence:
Protein Accession #: NP_005091

| | |
|---|---|
| 1 11 21 31 41 51 | |
| \| \| \| \| \| \| | |
| MGAGSSTSQR SPEQPPEGSS TPAEPEPSGG GPSAEAAPDT TADPAIAASD PATKLLQKNG | 60 |
| QLSTINGVAE QDELSLQEGD LNGQKGALNG QGALNSQEEE EVIVTEVGQR DSEDVSERDS | 120 |
| DKEMATKSAV VHDITDDGQE ENRNIEQIPS SESNLEELTQ PTESQANDIG FKKVFKFVGF | 180 |
| KFTVKKDKTE KPDTVQLLTV KKDEGEGAAG AGDHQDPSLG AGEAASKESE PKQSTEKPEE | 240 |
| TLKREQSHAE ISPPAESGQA VEECKEEGEE KQEKEPSKSA ESPTSPVTSE TGSTFKKFFT | 300 |
| QGWAGWRKKT SFRKPKEDEV EASEKEKEQE PEKVDTEEDG KAEVASEKLT ASEQANPQEP | 360 |
| AESAHEPRLS AEYEKVELPS EEQVSGSQGP SEEKPAPLAT EVFDEKIEVH QEEVVAEVHV | 420 |
| STVEERTEEQ KTEVEETAGS VPAEELVGMD AEPQEAEPAK ELVKLKETCV SGEDPTQGAD | 480 |
| LSPDEKVLSK PPEGVVSEVE MLSSQERMKV QGSPLKKLFT STGLKKLSGK KQKGKRGGGD | 540 |
| EESGEHTQVP ADSPDSQEEQ KGESSASSPE EPEEITCLEK GLAEVQQDGE AEEGATSDGE | 600 |
| KKREGVTPWA SFKKNVTPKK RVRRPSESDK EDELDKVKSA TLSSTESTAS EMQEEMKGSV | 660 |
| EEPKPEEPKR KVDTSVSWEA LICVGSSKKR ARRRSSSDEE GGPKAMGGDH QKADEAGKDE | 720 |
| ETGTDGILAG SQEHDPGQGS SSPEQAGSPT EGEGVSTWES FKRLVTPRKK SKSKLEEKSE | 780 |
| DSIAGSGVEN STPDTEPGKE ESWVSIKKFI PGRRKKRPDG KQEQAPVEDA GPTGANEDDS | 840 |
| DVPAVVPLSE YDAVEREKME AQQAQKGAEQ PEQKAATEVS KELSESQVHM MAAAVADGTR | 900 |
| AATIIEERSP SWISASVTEP LEQVEAEAAL LTEEVLEREV IAEEEPPTVT EPLPENREAR | 960 |
| GDTVVSEAEL TPEAVTAAET AGPLGSEEGT EASAAEETTE MVSAVSQLTD SPDTTEEATP | 1020 |
| VQEVEGGVPD IEEQERRTQE VLQAVAEKVK EESQLPGTGG PEDVLQPVQR AEAERPEEQA | 1080 |
| EASGLKKETD VVLKVDAQEA KTEPFTQGKV VGQTTPESFE KAPQVTESIE SSELVTTCQA | 1140 |
| ETLAGVKSQE MVMEQAIPPD SVETPTDSET DGSTPVADFD APGTTQKDEI VEIHEENEVA | 1200 |
| SGTQSGGTEA EAVPAQKERP PAPSSFVFQE ETKEQSKMED TLEHTDKEVS VETVSILSKT | 1260 |
| EGTQEADQYA DEKTKDVPFF EGLEGSIDTG ITVSREKVTE VALKGECTEE AECKKDDALE | 1320 |
| LQSHAKSPPS PVEREMVVQV EREKTEAEPT HVNEEKLEHE TAVTVSEEVS KQLLQTVNVP | 1380 |
| IIDGAKEVSS LEGSPPPCLG QEEAVCTKIQ VQSSEASFTL TAAAEEEKVL GETANILETG | 1440 |
| ETLEPAGANL VLEEKSSEKN EDFAAHPGED AVPTGPDCQA KSTPVIVSAT TKKGLSSDLE | 1500 |
| GEKTTSLKWK SDEVDEQVAC QEVKVSVAIE DLEPENGILE LETKSSKLVQ NIIQTAVDQF | 1560 |
| VETEETATEM LTSELQTQAH VIKADSQDAG QETEKEGEEP QASAQDETPI TSAKEESEST | 1620 |
| AVGQAHSDIS KDMSEASEKT MTVEVEGSTV NDQQLEEVVL PSEENGGGAG TKSVPEDDGN | 1680 |
| ALLAERIEKS LVEPKEDEKG DDVDGPENQN SALADTDASG GLTKESPDTN GPKQKEKEDA | 1740 |
| QEVELQEGKV HSESDKATTP QAQEELQKQE RESAKSELTE S | |

Seq ID NO: 45 Nucleotide sequence:
Nucleic Acid Accession #: NM_001290
Coding sequence: 110 . . . 1231 (underlined sequences correspond to
start and stop codons)

| | |
|---|---|
| 1 11 21 31 41 51 | |
| \| \| \| \| \| \| | |
| GTGAGCGTGT GTGCGTGCGT CTACTTTGTA CTGGGAAGAA CACAGCCCAT GTGCTCTGCA | 60 |
| TGGACGTTAC TGATACTCTG TTTAGCTTGA TTTTCGAAAA GCAGGCAAGA TGTCCAGCAC | 120 |
| ACCACATGAC CCCTTCTATT CTTCTCCTTT CGGCCCATTT TATAGGAGGC ATACACCATA | 180 |
| CATGGTACAG CCAGAGTACC GAATCTATGA GATGAACAAG AGACTGCAGT CTCGCACAGA | 240 |
| GGATAGTGAC AACCTCTGGT GGGACGCCTT TGCCACTGAA TTTTTTGAAG ATGACGCCAC | 300 |
| ATTAACCCTT TCATTTTGTT TGGAAGATGG ACCAAAGCGA TACACTATCG GCAGGACCCT | 360 |
| CATCCCCCGT TACTTTAGCA CTGTGTTTGA AGGAGGGGTG ACCGACCTGT ATTACATTCT | 420 |
| CAAACACTCG AAAGAGTCAT ACCACAACTC ATCCATCACG GTGGACTGCG ACCAGTGTAC | 480 |
| CATGGTCACC CAGCACGGGA AGCCCATGTT TACCAAGGTA TGTACAGAAG GCAGACTGAT | 540 |
| CTTGGAGTTC ACCTTTGATG ATCTCATGAG AATCAAAACA TGGCACTTTA GCATTAGACA | 600 |
| ATACCGAGAG TTAGTCCCGA GAAGCATCCT AGCCATGCAT GCACAAGATC CTCAGGTCCT | 660 |
| GGATCAGCTG TCCAAAAACA TCACCAGGAT GGGGCTAACA AACTTCACCC TCAACTACCT | 720 |
| CAGGTTGTGT GTAATATTGG AGCCAATGCA GGAACTGATG TCGAGACATA AAACTTACAA | 780 |
| CCTCAGTCCC CGAGACTGCC TGAAGACCTG CTTGTTTCAG AAGTGGCAGA GGATGGTGGC | 840 |
| TCCGCCAGCA GAACCCACAA GGCAACCAAC AACCAAACGG AGAAAAAGGA AAAATTCCAC | 900 |
| CAGCAGCACT TCCAACAGCA GCGCTGGGAA CAATGCAAAC AGCACTGGCA GCAAGAAGAA | 960 |
| GACCACAGCT GCAAACCTGA GTCTGTCCAG TCAGGTACCT GATGTGATGG TGGTAGGAGA | 1020 |
| GCCAACTCTG ATGGGAGGTG AGTTTGGGGA CGAGGACGAA AGGCTAATCA CTAGATTAGA | 1080 |
| AAACACGCAA TATGATGCGG CCAACGGCAT GGAGGAGGAG GAGGACTTCA ACAATTCACC | 1140 |
| CGCGCTGGGG AACAACAGCC CGTGGAACAG TAAACCTCCC GCCACTCAAG AGACCAAATC | 1200 |
| AGAAAACCCC CCACCCCAGG CTTCCCAATA AGATGATCGG CACCAGAATC CACTGTCAAT | 1260 |
| AGGCCCGTGG GTGATCATTA CAATTGCAAA TCTTTACTTA CAGGAGAGGA AACAGAAGAG | 1320 |
| ATAAAAACTT TTCCATGCAA ATATCTATTT CTAAACCACA ATGATCTGAT TTTCTTTCTT | 1380 |
| CTTTCTTTTT TTCTAATTGA GAGGATTATT CCCAGTAAGC TTCCATGACC CTTTCTTGGA | 1440 |
| GGCCTTCACA GGTAATACAG ATACTGGCAC TGATTGTAAT TAAAATGAGA GAAAACTCTA | 1500 |
| GCGCATCTTC TGGCACGGTT TTAACAACGT GTTTGTGTTG AATTTCCTTT TTATGCATCA | 1560 |
| AACGAAGGCC ATATTGTCCA TAAATGCTCA GTGCTCAGGA TCTCATTAAT ATGCCGAACC | 1620 |
| TAACTACAGA TGACTTTTTA ATATTGTAAA ATATTTTCTG CTTTTTGACT TGCATCTGAG | 1680 |
| AGTTTCTTGT TTCAGTAAAA AAAGAAAAGA CAAAAAAATC AGCTTTGGAA AGTAATTTAA | 1740 |
| ATGTACCTTA TTTTTTTTTT CTTTATGTTT TCTTTCATTG GGCAACAGCT AAGAGGGCCC | 1800 |
| AGCAAGGTAA TTTATGGTTG AGCTGATGTC AATTGGTTCT TGTCTTGAGT CGACTCAATT | 1860 |
| TAGCCCAAGT GCTGAAACAA GAAATGTCAT TTTTTTCATC AAAGACACCA GGGCAGATTT | 1920 |
| TTAAGTAAAG AAAGACAATT GGACCCTTAA GAATTTATGC ATTTGTAAAG TTGCTGTTGA | 1980 |
| TCCAAATATT TTCAAGCCAT GTAATCCATT GGTTTTGTGG GCAGTTTAAT AAACCTGAAC | 2040 |
| CTTTGTGTGT TTTCTAATTG TACCTGAGTT GACCATCCTT TCTTTTTATA GTATATTTCT | 2100 |
| TGTATGATAT TTTGTAAAGC TCTCACCTGG TTCTTTTATG GGGACTTTTC GTTTTTGGGC | 2160 |
| AACTCCAGTG TATTTATGTG AAACTTTATA AGAGAATTAA TTTTTCCATT TGCATATTAA | 2220 |
| TATGTTCCTC CACACATGTA AAGGCACAGT GGCTCCGTGT GTTAAAAAAC AGCTGTATTT | 2280 |
| TATGTATGCT TTACTGATAA GTGTGCCAAT AATAAACTGT GTTAATGACC | |

Seq ID NO: 46 Protein sequence:
Protein Accession #: NP_001281

| | |
|---|---|
| 1 11 21 31 41 51 | |
| \| \| \| \| \| \| | |
| MSSTPHDPFY SSPFGPFYRR HTPYMVQPEY RIYEMNKRLQ SRTEDSDNLW WGAFATEFFE | 60 |
| DDATLTLSFC LEDGPKRYTI GRTLIPRYFS TVFEGGVTDL YYILKNSKES YHNSSITVDC | 120 |
| DQCTMVTQHG KPMFTKVCTE GRLILEFTFD DLMRIKTWHF TIRQYRELVP RSILAMHAQD | 180 |
| PQVLDQLSKN ITRMGLTNFT LNYLRLCVIL EPMQELMSRH KTYNLSPRDC LKTCLFQKWQ | 240 |
| RMVAPPAEPT RQPTTKRRKR KNSTSSTSNS SAGNNANSTG SKKKTTAANL SLSSQVPDVM | 300 |
| VVGEPTLMGG EFGDEDERLI TRLENTQYDA ANGMDDEEDF NNSPALGNNS PWNSKPPATQ | 360 |
| ETKSENPPPQ ASQ | |

Seq ID NO: 47 Nucleotide sequence:
Nucleic Acid Accession #: NM_004126
Coding sequence: 108 . . . 329 (underlined sequences correspond to
start and stop codons)

| | |
|---|---|
| 1 11 21 31 41 51 | |
| \| \| \| \| \| \| | |
| GGCACGAGCT CGTGCCGGCC TTCAGTTGTT TCGGGACGCG CCGAGCTTCG CCGCTCTTCC | 60 |
| AGCGGCTCCG CTGCCAGAGC TAGCCCGAGC CCGGTTCTGG GGCGAAAATG CCTGCCCTTC | 120 |
| ACATCGAAGA TTTGCCAGAG AAGGAAAAAC TGAAAATGGA AGTTGAGCAG CTTCGCAAAG | 180 |
| AAGTGAAGTT GCAGAGACAA CAAGTGTCTA AATGTTCTGA AGAAATAAAG AACTATATTG | 240 |
| AAGAACGTTC TGGAGAGGAT CCTCTAGTAA AGGGAATTCC AGAAGACAAG AACCCCTTTA | 300 |
| AAGAAAAAGG CAGCTGTGTT ATTTCATAAA TAACTTGGGA GAAACTGCAT CCTAAGTGGA | 360 |
| AGAACTAGTT TGTTTTAGTT TTCCCAGATA AAACCAACAT GCTTTTTAAG GAAGGAAGAA | 420 |
| TGAAATTAAA AGGAGACTTT CTTAAGCACC ATATAGATAG GGTTATGTAT AAAAGCATAT | 480 |
| GTGCTACTCA TCTTTGCTCA CTATGCAGTC TTTTTTAAGA GAGCAGAGAG TATCAGATGT | 540 |
| ACAATTATGG AAATAAGAAC ATTACTTGAG CATGACACTT CTTTCAGTAT ATTGCTTGAT | 600 |
| GCTTCAAATA AAGTTTTGTC TT | |

Seq ID NO: 48 Protein sequence:
Protein Accession #: NP_004117

| | |
|---|---|
| 1 11 21 31 41 51 | |
| \| \| \| \| \| \| | |
| MPALNIEDLP EKEKLKMEVE QLRKEVKLQ RQQVSKCSEEI KNYIEERSGE GPLVKGIPED | 60 |
| KNPFKEKGSC VIS | |

Seq ID NO: 49 Nucleotide sequence:
Nucleic Acid Accession #: XM_051896
Coding sequence: 139 . . . 2388 (underlined sequences correspond to
sturt and stop codons)

| | |
|---|---|
| 1 11 21 31 41 51 | |
| \| \| \| \| \| \| | |
| GTTTTAAAGA CGCTAGAGTG CCAAAGAAGA CTTTGAAGTG TGAAAACATT TCCTGTAATT | 60 |
| GAAACCAAAA TGTCATTTAT AGATCCTTAC CAGCACATTA TAGTGGAGCA CCAGTATTCC | 120 |
| CACAAGTTTA CGGTAGTGGT GTTACGTGCC ACCAAAGTGA CAAAGGGGGC CTTTGGTGAC | 180 |
| ATGCTTGATA CTCCAGATCC CTATGTGGAA CTTTTTATCT CTACAACCCC TGACAGCAGG | 240 |
| AAGAGAACAA GACATTTCAA TAATGACATA AACCCTGTGT GGAATGAGAC CTTTGAATTT | 300 |
| ATTTTGGATC CTAATCAGGA AAATGTTTTG GAGATTACGT TAATGGATGC CAATTATGTC | 360 |
| ATGGATGAAA CTCTAGGGAC AGCAACATTT ACTGTATCTT CTATGAAGGT GGGAGAAAAG | 420 |
| AAAGAAGTTC CTTTTATTTT CAACCAAGTC ACTGAAATGG TTCTAGAAAT GTCTCTTGAA | 480 |
| GTTTGCTCAT GCCCAGACCT ACGATTTAGT ATGGCTGTGT GTCATCAGGA GAAGACTTTC | 540 |
| AGACAACAGA GAAAAGAACA CATAAGGGAG AGCATGAAGA AACTCTTGGG TCCAAAGAAT | 600 |
| AGTGAAGGAT TGCATTCTGC ACGTGATGTG CCTGTGGTAG CCATATTGGG TTCAGGTGGG | 660 |
| GGTTTCCGAG CCATGGTGGG ATTCTCTGGT GTGATGAAGG CATTATACGA ATCAGGAATT | 720 |
| CTGGATTGTG CTACCTACGT TGCTGGTCTT TGTGGCTCCA CCTGGTATAT GTCAACCTTG | 780 |
| TATTCTCACC CTGATTTTCC AGAGAAAGGG CCAGAGGAGA TTAATGAAGA ACTAATGAAA | 840 |
| AATGTTAGCC ACAATCCCCT TTTACTTCTC ACACCACAGA AAGTTAAAAG ATATGTTGAG | 900 |
| TCTTTATGGA AGAAGAAAAG CTCTGGACAA CCTGTCACCT TTACTGATAT CTTTGGGATC | 960 |
| TTAATACGAG AAACACTAAT TCATAATAGA ATGAATACTA CTCTGACCAG TTTGAAGGAA | 1020 |
| AAAGTTAATA CTGCACAATG CCCTTTACCT CTTTTCACCT GTCTTCATGT CAAACCTGAC | 1080 |
| GTTTCAGAGC TGATGTTTGC AGATTGGGTT GAATTTAGTC CATACGAAAT TGGCATGGCT | 1140 |
| AAATATGGTA CTTTTATGGC TCCCGACTTA TTTGGAAGCA AATTTTTTAT GGGAACAGTC | 1200 |
| GTTAAGAAGT ATGAAGAAAA CCCCTTGCAT TTCTTAATGG GTGTCTGGGG CAGTGCCTTT | 1260 |
| TCCATATTGT TCAACAGAGT TTTGGGCGTT TCTGGTTCAC AAAGCAGAGG CTCCACAATG | 1320 |
| GAGGAAGAAT TAGAAAATAT TACCACAAAG CATATTGTGA GTAATGATAG CTCGGACAGT | 1380 |
| GATGATGAAT CACACGAACC CAAAGGCACT GAAAATGAAG ATGCTGGAAG TGACTATCAA | 1440 |
| AGTGATAATC AAGCAAGTTG GATTCATCGT ATGATAATGG CCTTGGTGAG TGATTCAGCT | 1500 |
| TTATTCAATA CCAGAGAAGG ACGTGCTGGG AAGGTACACA ACTTCATGCT GGGCTTGAAT | 1560 |
| CTCAATACAT CTTATCCACT GTCTCCTTTG AGTGACTTTG CCACACAGGA CTCCTTTGAT | 1620 |
| GATGATGAAC TGGATGCAGC TGTAGCAGAT CCTGATGAAT TTGAGCGAAT ATATGAGCCT | 1680 |
| CTGGATGTCA AAAGTAAAAA GATTCATGTA GTGGACAGTG GGCTCACATT TAACCTGCCG | 1740 |
| TATCCCTTGA TACTGAGACC TCAGAGAGGG GTTGATCTCA TAATCTCCTT TGACTTTTCT | 1800 |
| GCAAGGCCAA GTGACTCTAG TCCTCCGTTC AAGGAACTTC TACTTGCAGA AAAGTGGGCT | 1860 |
| AAAATGAACA AGCTCCCCTT TCCAAAGATT GATCCTTATG TGTTTGATCG GGAAGGGCTG | 1920 |
| AAGGAGTGCT ATGTCTTTAA ACCCAAGAAT CCTGATATGG AGAAAGATTG CCCAACCATC | 1980 |
| ATCCACTTTG TTCTGGCCAA CATCAACTTC AGAAAGTACA GGGCTCCAGG TGTTCCAAGG | 2040 |
| GAAACTGAGG AAGAGAAAGA AATCGCTGAC TTTGATATTT TTGATGACCC AGAATCACCA | 2100 |
| TTTTCAACCT TCAATTTTCA ATATCCAAAT CAAGCATTCA AAAGACTACA TGATCTTATG | 2160 |
| CACTTCAATA CTCTGAACAA CATTGATGTG ATAAAAGAAG CCATGGTTGA AAGCATTGAA | 2220 |
| TATAGAAGAC AGAATCCATC TCGTTGCTCT GTTTCCCTTA GTAATGTTGA GGCAAGAAGA | 2280 |
| TTTTTCAACA AGGAGTTTCT AAGTAAACCC AAAGCATAGT TCATGTACTG GAAATGGCAG | 2340 |
| CAGTTTCTGA TGCTGAGGCA GTTTGCAATC CCATGACAAC TGGATTTAAA AGTACAGTAC | 2400 |
| AGATAGTCGT ACTGATCATG AGAGACTGGC TGATACTCAA AGTTGCAGTT ACTTAGCTGC | 2460 |
| ATGAGAATAA TACTATTATA AGTTAGGTTG ACAAATGATG TTGATTATGT AAGGATATAC | 2520 |
| TTAGCTACAT TTTCAGTCAG TATGAACTTC CTGATACAAA TGTAGGGATA TATACTGTAT | 2580 |
| TTTTAAACAT TTCTCACCAA CTTTCTTATG TGTGTTCTTT TTAAAAATTT TTTTTCTTTT | 2640 |
| AAAATATTTA ACAGTTCAAT CTCAATAAGA CCTCGCATTA TGTATGAATG TTATTCACTG | 2700 |
| ACTAGATTTA TTCATACCAT GAGACAACAC TATTTTTATT TATATATGCA TATATATACA | 2760 |
| TACATGAAAT AAATACATCA ATATAAAAAT | |

Seq ID NO: 50 Protein sequence:
Protein Accession #: XP_051896

| | |
|---|---|
| 1 11 21 31 41 51 | |
| \| \| \| \| \| \| | |
| MSFIDPYQHI IVEHQYSHKF TVVVLRATKV TKGAFGGMLD TPDPYVELFI STTPDSRKRT | 60 |
| RHFNNDINPV WNETFEFILD PNQENVLEIT LMDANYVMDE TLGTATFTVS SMKVGEKKEV | 120 |
| PFIFNQVTEM VLEMSLEVCS CPDLRFSMAL CDQEKTFRQQ RKEHIRESMK KLLGPKNSEG | 180 |
| LHSARDVPVV AILGSGGGFR ANVGFSGVMK ALYESGILDC ATYVAGLSGS TWYMSTLYSH | 240 |
| PDFPEKGPEE INEELMKNVS HNPLLLLTPQ KVKRYVESLW KKKSSGQPVT FTDIFGMLIG | 300 |
| ETLIHNRMNT TLSSLKEKVN TAQCPLPLFT CLHVKPDVSE LMFADWVEFS PYEIGMAKYG | 360 |
| TFMAPDLFGS KFFMGTVVKK YEENPLHFLM GVWGSAFSIL FNRVLGVSGS QSRGSTMEEE | 420 |
| LENITTKHIV SNDSSDSDDE SHEPKGTENE DAGSDYQSDN QASWIHRMIM ALVSDSALFN | 480 |
| TREGRAGKVH NFMLGLNLNT SYPLSPLSDF ATQDSFDDDE LDAAVADPDE FERIYEPLDV | 540 |
| KSKKIHVVDS GLTFNLPYPL ILRPQRGVDL IISFDFSARP SDSSPPFKEL LLAEKWAKMN | 600 |
| KLPFPKIDPY VFDREGLKEC YVFKPKNPDM EKDCPTIIHF VLANINFRKY KAPGVPRETE | 660 |
| EEKEIADFDI FDDPESPFST PNFQYPNQAF KRLHDLMHFN TLNNIDVIKE AMVESIEYRR | 720 |
| QNPSRCSVSL SNVEARRFFN KEFLSKPKA | |

Seq ID NO: 51 Nucleotide sequence:
Nucleic Acid Accession #: NM_006528
Coding sequence: 57 . . . 764 (underlined sequences correspond to
start and stop codons)

| | |
|---|---|
| 1 11 21 31 41 51 | |
| \| \| \| \| \| \| | |
| GCCGCCAGCG GCTTTCTCGG ACGCCTTGCC CAGCGGGCCG CCCGACCCCC TGCACCATGG | 60 |
| ACCCCGCTCG CCCCCTGGGG CTGTCGATTC TGCTGCTTTT CCTGACGGAG GCTGCACTGG | 120 |
| GCGATGCTGC TCAGGAGCCA ACAGGAAATA ACGCGGAGAT CTGTCTCCTG CCCCTAGACT | 180 |
| ACGGACCCTG CCGGGCCCTA CTTCTCCGTT ACTACTACGA CAGGTACACG CAGAGCTGCC | 240 |
| GCCAGTTCCT GTACGGGGGC TGCGAGGGCA ACGCCAACAA TTTCTACACC TGGGAGGCTT | 300 |
| GCGACGATGC TTGCTGGAGG ATAGAAAAAG TTCCCAAAGT TTGCCGGCTG CAAGTGAGTG | 360 |
| TGGACGACCA GTGTGAGGGG TCCACAGAAA AGTATTTCTT TAATCTAAGT TCCATGACAT | 420 |
| GTGAAAAATT CTTTTCCGGT GGGTGTCACC GGAACCGGAT TGAGAACAGG TTTCCAGATG | 480 |
| AAGCTACTTG TATGGGCTTC TGCGCACCAA AGAAAATTCC ATCATTTTGC TACAGTCCAA | 540 |
| AAGATGAGGG ACTGTGCTCT GCCAATGTGA CTCGCTATTA TTTTAATCCA AGATACAGAA | 600 |
| CCTGTGATGC TTTCACCTAT ACTGGCTGTG GAGGGAATGA CAATAACTTT GTTAGCAGGG | 660 |
| AGGATTGCAA ACGTGCATGT GCAAAAGCTT TGAAAAAGAA AAAGAAGATG CCAAAGCTTC | 720 |
| GCTTTGCCAG TAGAATCCGG AAAATTCGGA AGAAGCAATT TTAACATTC TTAATATGTC | 780 |
| ATCTTGTTTG TCTTTATGGC TTATTTGCCT TTATGGTTGT ATCTGAAGAA TAATATGACA | 840 |
| GCATGAGGAA ACAAATCATT GGTGATTTAT TCACCAGTTT TTATTAATAC AAGTCACTTT | 900 |
| TTCAAAAATT TGGATTTTTT TATATATAAC TAGCTGCTAT TCAAATGTGA GTCTACCATT | 960 |
| TTTAATTTAT GGTTCAACTG TTTGTGAGAC GAATTCTTGC AATGCATAAG ATATAAAAGC | 1020 |
| AAATATGACT CACTCATTTC TTGGGGTCGT ATTCCTGATT TCAGAAGAGG ATCATAACTG | 1080 |
| AAACAACATA AGACAATATA ATCATGTGCT TTTAACATAT TTGAGAATAA AAAGGACTAG | 1140 |
| CC | |

Seq ID NO: 52 Protein sequence:
Protein Accession #: NP_006519

| | |
|---|---|
| 1 11 21 31 41 51 | |
| \| \| \| \| \| \| | |
| MDPARPLGLS ILLLFLTEAA LGDAAQEPTG NNAEICLLPL DYGPCRALLL RYYYDRYTQS | 60 |
| CRQFLYGGCE GNANNFYTWE ACDDACWRIE KVPKVCRLQV SVDDQCEGST EKYFFNLSSM | 120 |
| TCEKFFSGGC HRNRIENRFP DEATCMGFCA PKKIPSFCYS PKDEGLCSAN VTRYYFNPRY | 180 |
| RTCDAFTYTG CGGNDNNFVS REDCKRACAK ALKKKKKMPK LRFASRIRKI RKKQF | |

Seq ID NO: 53 Nucleotide sequence:
Nucleic Acid Accession #: AA478778
Coding sequence: no ORF found

| | |
|---|---|
| 1 11 21 31 41 51 | |
| \| \| \| \| \| \| | |
| TATTTTTGTA CGTAAAATGA TTCTATTATG ACTGCCTTTG CATGTAGTAA TATGACAAAG | 60 |
| TGATCCTTCA TTATCACGGT ACACTATTGT TTACTTTTCA TCTGTAAATG TTTTATTGTT | 120 |
| ACTTTTTTAA AATGAATTTT TTTAAAACAA TCTAGCCATC ATCAAGGTGC TATAAGAGTT | 180 |
| GTATAAAAGA TATTTTTGGC ATTTCTAGGC AAGTATCAGC CAATAAGTAT GTTAGTGATA | 240 |
| TCACAGATTG TACCAACTAT TAACTATGTT AAATAAGTAT TCAGTTTCAT GTGATCTCTG | 300 |
| GGAAAAAAAT ATGCTGCCTT GGTGCTAATA TTGTATGTAT TTAAATGATC ATCTGACTCA | 360 |
| GAAATATAAA CACTTTTAAT GAAAGGGAGG AACGGAAGGA CAATTTCCAG TGCACAGAAT | 420 |
| CACTTGGATG AAATAAGACC AGCTCTTTAC CCTTATTTTT GGATATGCCT TTTTTGGAAG | 480 |
| AGACTTAGAC TTTATCCTTA TTGTTGTTAG TGTTGTTAAT ATTCGTTGCT TCAGCCCACG | 540 |
| GTGCCTTGGT CTCTCCACAA TCAAATGGAG GATCCCCCAA GCAGCTTCAT TACAGAGTGA | 600 |
| TATTGGGAAA GTGAGATCCT CTCACCATTT TGCCAAGATA CTCTAAAATG ACATCCAAGT | 660 |
| TTACCAGTAG AAAGACACAG GATGCACAGA ATGGGCATGA CCTTCAGCTC ACGAGCACAC | 720 |
| CTGGAGAAAT TCAGAACCAG GTTCTGAATC ATCACGATTG CCTTTTGCAT GAAAACATCG | 780 |
| GCTGGTGATG TGACTTCTCT TCAGGCCATG AGCCTAACAY CCTGCCGGTT TTCATGCCCG | 840 |
| CTGCAGTAAT GGACGTTTGT GTGAAGAAAT GAACTGTGGA GTACAAAATG CTTTGAGTCT | 900 |
| TTCCGATTGC TCATTAATTC ACTTTTTTGT TACTTCTTTC CAAAATGGAA GTGCTGAAGC | 960 |
| CATGGTCTTT CTGCCCCTCC AAGCTGATGA AGGGAAGCCT TTGCCAATGG CCCATGGAAG | 1020 |
| ACACTTGGTT TGAGAAACCC TGCCCACTTC CAAAGACCAA AGAGATTAGG AAAAGCCTGG | 1080 |
| CAGTATTCTC CAACTCCAAA CAAGCTCTAG AGTGCTCCAG GAAAAGTTAT ATTCAGTATA | 1140 |
| TGAATAAGTG TTATTCTCCA TTATTAATGT GTTCTGAAAA TATATTATGA ATAAATACAT | 1200 |
| CACCACACCC AAAAAAAAAA AAAAAAAAAA AAAA | |

Seq ID NO: 54 Nucleotide sequence:
Nucleic Acid Accession #: NM_020663
Coding sequence: 1 . . . 645 (underlined sequences correspond to start
and stop codons)

| | |
|---|---|
| 1 11 21 31 41 51 | |
| \| \| \| \| \| \| | |
| ATGAACTGCA AAGAGGGAAC TGACAGCAGC TGCGGCTGCA GGGGCAACGA CGAGAAGAAG | 60 |
| ATGTTGAAGT GTGTGGTGGT GGGGGACGGT GCCGTGGGGA AAACCTGCCT GCTGATGAGC | 120 |
| TACGCCAACG ACGCCTTCCC AGAGGAATAC GTGCCCACTG TGTTTGACCA CTATGCAGTT | 180 |
| ACTGTGACTG TGGGAGGCAA GCAACACTTG CTCGGACTGT ATGACACCGC GGGACAGGAG | 240 |
| GACTACAACC AGCTGAGGCC ACTCTCCTAC CCCAACACGG ATGTGTTTTT GATCTGCTTC | 300 |
| TCTGTCGTAA ACCCTGCCTC TTACCACAAT GTCCAGGAGG AATGGGTCCC CGAGCTCAAG | 360 |
| GACTGCATGC CTCACGTGCC TTATGTCCTC ATAGGGACCC AGATTGATCT CCGTGATGAC | 420 |
| CCAAAAACCT TGGCCCGTTT GCTGTATATG AAAGAGAAAC CTCTCACTTA CGAGCATGGT | 480 |
| GTGAAGCTCG CAAAAGCGAT CGGAGGACAG TGCTACTTGG AATGTTCAGC TCTGACTCAG | 540 |
| AAAGGTCTCA AAGCGGTTTT TGATGAAGCA ATCCTCACCA TTTTCCACCC CAAGAAAAAG | 600 |
| AAGAAACGCT GTTCTGAGGG TCACAGCTGC TGTTCAATTA TCTGA | |

Seq ID NO: 55 Protein sequence:
Protein Accession #: NP_065714

| | |
|---|---|
| 1 11 21 31 41 51 | |
| \| \| \| \| \| \| | |
| MNCKEGTDSS CGCRCNDEKK MLKCVVVGDG AVGKTCLLMS YANDAFPEEY VPTVFDHYAV | 60 |
| TVTVGGKQHL LGLYDTAGQE DYNQLRPLSY PNTDVFLICF SVVNPASYHN VQEEWVPELK | 120 |
| DCMPHVPYVL IGTQIDLRDD PKTLARLLYM KEKPLTYEHG VKLAKAIGAQ CYLECSALTQ | 180 |
| KGLKAVFDEA ILTIFHPKKK KKRCSEGHSC CSII | |

Seq ID NO: 56 Nucleotide sequence:
Nucleic Acid Accession #: fgenesh prediction
Coding sequence: 1-546 (underlined sequences correspond to start
and stop codons)

| | |
|---|---|
| 1 11 21 31 41 51 | |
| \| \| \| \| \| \| | |
| ATGGCCTTGG GCAGCTCCGC CCCTGTGGCT TTGCAGGGTA ATGCCCACTT CCCTGCTGCT | 60 |
| TTCATGGCTG GCATTAAGTG TCTGTGGCTT TTCCAGGTAG TCCCCCTGGG GCTCCCCGAG | 120 |
| TTGGTGCAAA GGCTCCTGGG TGGAGCTCGA ACTGAAACTC GCTTTGTGCC CGCAGCCCTG | 180 |
| CAGCTCGCCG GTGCCCTCGA CCTGCCCGCT GGGTCCTGTG CCTTTGAAGA GAGCACTTGC | 240 |
| GGCTTTGACT CCGTGTTGGC CTCTCTGCCG TGGATTTTAA ATGAGGAAGG CCAGCAACCT | 300 |
| TTCTGGTCCT CAGGAGACAT GTCTGACTGG GACTACTGGG TTGGCTGGCG GAAGTTAATT | 360 |
| CATTCTCCTC TGAGCACTCC AGGGTGGAGC AGGCAGGTTA GGCTCCAGTT GTTCCAGCTT | 420 |
| CAGTTTGTCA AAGGCCAGAA CTTGGACGTA ACAGTGTACT GCAGGCTCCA GGGCAGTGAG | 480 |
| AAACCCTTTG AAACTGGTTC CATGGTTCCA TTCACCTTCA TGTACTGGAT CCACCATGGA | 540 |
| AAGTAG | |

Seq ID NO: 57 Protein sequence:
Protein Accession #: fgenesh prediction

| | |
|---|---|
| 1 11 21 31 41 51 | |
| \| \| \| \| \| \| | |
| MALGSSAPVA LQGNAHFPAA FMAGIKCLWL FQVVPLGLPE LVQRLLGGAR TETRFVPAAL | 60 |
| QLAGALDLPA GSCAFEESTC GFDSVLASLP WILNEEGQQP FWSSGDMSDW DYWVGWRKLI | 120 |
| HSPLSTPGWS RQVRLQLFQL QFVKGQNLDV TVYCRLQGSE KPFETGSMVP FTFMYWIHHG | 180 |
| K | |

Seq ID NO: 58 Nucleotide sequence:
Nucleic Acid Accession #: XM_050478
Coding sequence: 27 . . . 4508 (underlined sequences correspond to
start and stop codons)

| | |
|---|---|
| 1 11 21 31 41 51 | |
| \| \| \| \| \| \| | |
| CCGGCGGCGC CTGAGCCCAG CCGAGGATGG AGAACCGGCC TGGGTCCTTC CAGTACGTCC | 60 |
| CTGTGCAGCT GCAAGGGGGG GCACCCTGGG GCTTCACCCT TAAGGGGGGT CTGGAACACT | 120 |
| GTGAGCCGCT CACAGTGTCT AAGATTGAAG ATGGAGGCAA GGCAGCTTTG TCCCAGAAGA | 180 |
| TGAGGACTGG TGATGAGCTG GTGAATATCA ATGGCACTCC ATTATATGGC TCCCGCCAAG | 240 |
| AGGCCCTCAT TCTCATCAAA GGCTCCTTCC GGATTCTCAA GCTGATTGTC AGGAGGAGGA | 300 |
| ACGCCCCTGT CAGTAGGCCG CACTCATGGC ATGTGGCCAA GCTGCTGGAG GGATGCCCTG | 360 |
| AAGCAGCCAC CACCATGCAT TTCCCTTCTG AAGCCTTCAG CTTGTCCTGG CATTCTGGCT | 420 |
| GCAACACAAG TGACGTGTGT GTGCAGTGGT GTCCACTCTC CCGGCATTGC AGCACCGAGA | 480 |
| AAAGCAGCTC CATTGGCAGC ATGGAGAGCC TGGAGCAACC AGGCCAAGCC ACCTATGAGA | 540 |
| GCCATCTGTT GCCTATTGAC CAGAACATGT ACCCTAACCA GCGTGACTCA GCCTACAGCT | 600 |
| CCTTCTCGGC CAGCTCAAAT GCTTCTGACT GTGCCCTTTC CCTCAGGCCA GAGGAGCCAG | 660 |
| CCTCTACAGA CTGCATCATG CAAGGCCCAG GGCCAACTAA GGCCCCCAGT GGCCGGCCTA | 720 |
| ATGTGGCTGA GACCTCAGGA GGTAGTCGGC GCACCAATGG GGGCCACCTG ACCCCCAGCT | 780 |
| CTCAGATGTC ATCCCGTCCA CAGGAGGGAT ACCAGTCAGG GCCCGCCAAA GCAGTCAGGG | 840 |
| GCCCACCACA ACCTCCAGTG AGGCGGGACA GCCTTCAGGC CTCCAGAGCC CAACTCCTCA | 900 |
| ATGGAGAGCA GCGCAGGGCA TCTGAGCCTG TGGTCCCCTT GCCACAGAAG GAGAAACTGA | 960 |
| GCTTAGAGCC TGTGCTACCC GCAAGGAACC CTAATAGGTT CTGTTGCCTC AGTGGGCATG | 1020 |
| ACCAAGTGAC AAGTGAGGGC CATCAGAACT GTGAGTTCAG TCAGCCTCCT GAATCCAGCC | 1080 |
| AACAGGGCTC TGAGCATCTA CTGATGCAGG CCTCAACCAA AGCTGTTGGA TCCCCAAAAG | 1140 |
| CCTGTGACAG AGCTTCCAGC GTGGATTCCA ACCCACTCAA TGAGGCTTCT GCAGAGCTAG | 1200 |
| CTAAGGCTTC TTTTGGCAGA CCTCCACATC TCATAGGACC CACAGGGCAT CGCCATAGTG | 1260 |
| CCCCTGAACA GCTGCTGGCA TCCCACCTGC AGCATGTGCA CCTTGATACC AGGGGCAGCA | 1320 |
| AAGGGATGGA GCTCCCACCC GTACAGGATG GGCACCAGTG GACTCTGTCC CCTTTGCACA | 1380 |
| GCAGCCACAA AGGGAAGAAA AGTCCATGCC CCCCTACAGG AGGAACCCAT GACCAGTCCA | 1440 |
| GCAAAGAAAG AAAGACCAGA CAAGTGGATG ACAGGTCTTT AGTTTTGGGA CACCAGAGCC | 1500 |
| AAAGCAGTCC CCCACATGGA GAGGCTGATG GACACCCCTC AGAAAAAGGT TTCCTGGACC | 1560 |
| CAAACAGAAC AAGCAGAGCA GCCAGTGAAT TGGCCAACCA GCAACCCTCT GCCTCTGGCT | 1620 |
| CCCTTGTTCA ACAAGCCACG GACTGTTCTT CAACCACTAA AGCAGCTAGT GGCACAGAGG | 1680 |
| CAGGTGAAGA AGGGGACAGC GAgCCCAAGG AGTGCAGCCG GATGGGTGGT AGGCGAAGTG | 1740 |
| GAGGGACCCG GGGCCGCTCG ATCCAAAACC GGCGGAAGAG TGAGCGTTTT GCTACCAATC | 1800 |
| TGCGTAATGA AATTCAGAGG AGGAAGGCCC AGCTCCAGAA AAGCAAGGGT CCCTTGTCAC | 1860 |
| AGCTGTGTGA CACTAAGGAG CCAGTGGAAG AGACCCAGGA GCCCCCAGAA AGTCCTCCAC | 1920 |
| TCACTGCCTC TAACACATCT CTTCTATCTT CATGTAAAAA ACCTCCCAGC CCCAGAGACA | 1980 |
| AGCTCTTCAA CAAAAGCATG ATGCTCAGAG CTAGGTCTTC CGAGTGCCTC AGCCAAGCCC | 2040 |
| CTGAGAGCCA TGAATCTAGG ACAGGCTTAG AGGGACGAAT AAGCCCTGGC CAGAGGCCTG | 2100 |
| GCCAGTCCTC TTTGGGCCTG AACACCTGGT GGAAAGCACC TGACCCATCC TCCTCAGACC | 2160 |
| CTGAGAAAGC ACATGCTCAC TGTGGAGTCC GTGGAGGTCA TTGGAGATGG TCTCCAGAGC | 2220 |
| ATAATTCACA GCCACTTGTG GCAGCAGCCA TGGAAGGCCC TTCCAACCCA GGTGACAACA | 2280 |
| AGGAATTGAA GGCTTCTACT GCTCAAGCTG GGGAGGATGC CATCCTCTTG CCTTTTGCAG | 2340 |
| ACAGAAGAAA GTTCTTTGAA GAGAGTAGGA AATCCTTATC TACATCTCAT TTGCCAGGTT | 2400 |
| TAACCACTCA TAGCAACAAG ACTTTTACCC AGAGACCAAA ACCTATAGAC CAAAACTTCC | 2460 |
| AGCCAATGAG CTCCAGCTGT AGGGAATTGA GGCGCCATCC CATGGACCAA TCATATCATT | 2520 |
| CCGCAGACCA ACCATATCAT GCCACAGACC AATCATATCA TTCCATGTCA CCCCTTCAGT | 2580 |
| CAGAAACTCC CACTTACTCA GAATGTTTTG CAAGCAAAGG TCTAGAAAAT TCCATGTGTT | 2640 |
| GTAAGCCACT ACACTGTGGT GATTTTGATT ACCACAGGAC CTGCTCTTAC TCCTGCAGTG | 2700 |
| TTCAAGGAGC TCTAGTCCAT GATCCTTGCA TTTATTGTTC TGGGGAAATC TGCCCTGCCT | 2760 |
| TGCTAAAGAG AAATATGATG CCAAATTGCT ACAACTGCCG GTGCCACCAC CACCAATGCA | 2820 |
| TTCGGTGTTC AGTTTGCTAT CATAATCCTC AGCACAGTGC CCTCGAGGAC AGCAGCTTGG | 2880 |
| CACCTGGCAA CACTTGGAAA CCCAGGAAGC TGACAGTGCA GGAATTTCCT GGGGACAAAT | 2940 |
| GGAATCCAAT AACAGGAAAC AGGAAGACCA GCCAGTCAGG GAGGGAAATG GCTCATTCCA | 3000 |
| AGACTAGCTT TTCATGGGCA ACCCCTTTCC ATCCTTGCCT TGAGAACCCA GCACTGGACT | 3060 |
| TGTCAAGCTA CCGAGCAATT TCTTCTCTTG ACCTCCTTGG AGACTTCAAA CATGCTTTGA | 3120 |
| AAAAATCAGA GGAAACTTCA GTTTATGAGG AGGGGAGCTC CCTTGCCTCC ATGCCCCACC | 3180 |
| CACTGCGCAG CCGTGCCTTC TCAGAGAGTC ACATCAGCTT GGCGCCCCAA AGCACCCGGG | 3240 |
| CCTGGGGGCA GCATAGGAGG GAGCTCTTTA GCAAAGGTGA TGAGACCCAG TCGGATCTTC | 3300 |
| TCGGAGCCAG GAAGAAGGCC TTTCCTCCTC CTCGCCCTCC TCCTCCCAAC TGGGAGAAGT | 3360 |
| ACAGGCTCTT TCGTGCAGCC CAGCAGCAGA AGCAGCAACA GCAGCAGCAG AAGCAACAGG | 3420 |
| AGGAGGAGGA GGAGGAGGAA GAAGAAGAAG AAGAGGAAGA GGAAGAGGAG GAGGAGGAGG | 3480 |
| CAGAGGAGGA GGAAGAGGAG CTGCCACCCC AGTATTTCAG TTCAGAAACC TCTGGTTCCT | 3540 |
| GTGCTCTCAA TCCTGAGGAG GTCCTAGAGC AGCCACAACC CCTCAGCTTT GGCCACCTGG | 3600 |
| AGGGCTCGAG ACAGGGTTCA CAAAGTGTCC CAGCAGAGCA AGAATCCTTT GCACTCCATT | 3660 |
| CCAGTGATTT CTTGCCTCCA ATAAGGGGTC ACTTGGGATC TCAACCTGAG CAGGCTCAGC | 3720 |
| CCCCTTGCTA CTATGGCATT GGTGGGCTTT GGAGGACATC GGGACAGGAA GCCACTGAAT | 3780 |
| CCGCCAAACA AGAGTTTCAG CACTTTTCGC CTCCTTCAGG GGCCCCAGGA ATCCCTACCT | 3840 |
| CTTACTCAGC TTATTACAAT ATTTCTGTGG CCAAGGCAGA GCTGCTGAAC AAACTGAAAG | 3900 |
| ACCAACCTGA GATGGCAGAG ATTGGCCTAG GAGAGGAGGA AGTTGACCAT GAACTGGCTC | 3960 |
| AAAAAAAGAT ACAGCTTATC GAAAGCATCA GCAGAAAACT TTCTGTCTTG CGGGAGGCCC | 4020 |
| AGCGAGGGCT GCTAGAGGAC ATCAATGCCA ATTCTGCCCT TGGGGAGGAG GTGGAGGCCA | 4080 |
| ACTTAAAAGC CGTCTGCAAA TCCAATGAAT TTGAAAAGTA CCACTTGTTT GTTGGGGACC | 4140 |
| TGGACAAAGT GGTCAACCTG TTGCTGTCAC TCTCTGGACG ACTGGCCCGG GTGGAGAATG | 4200 |
| CTCTGAACAG CATCGATTCA GAGGCCAACC AGGAGAAGTT GGTACTGATA GAGAAGAAGC | 4260 |
| AGCAGCTGAC GGGGCAGTTG GCAGATGCCA AGGAGCTGAA GGAGCACGTG GACCGCCGGG | 4320 |
| AGAAGTTGGT GTTTGGCATG GTCTCCCGCT ACCTGCCTCA GGACCAGCTC CAAGATTACC | 4380 |
| AGCACTTTGT CAAGATGAAA TCTGCTCTCA TCATTGAACA GCGAGAGCTG GAGGAGAAGA | 4440 |
| TCAAGCTCGG GGAAGAGCAA CTCAAATGTC TCAGGGAGAG TCTACTCCTG GGGCCCAGCA | 4500 |
| ATTTCTAATT CTACCAGCAC TCTGCCACAG CATCCCTGCC CAGCCATGTG GGAAGTGCTT | 4560 |
| TCAATCTTCT TTGTTAGCAG TTTCTCAGCA AGTAGATAGC AATTAGCAGT TTGTTCCAGC | 4620 |
| CCTCTACCCT GGATGTCTCT CACTACCCCT TCCCTAGCAG TGGTCCTAAC CAGCTAGGAG | 4680 |
| ACCCTGGGGA AGCCACAAGC TTCTACCCAA GGGAGCTGCA GCAAGGTGTG ATCTTAGAAC | 4740 |
| CACACTCTCC TTCCCACAGT TGCCAAGGGC AAGTACTTGC TGCACAGAGA ACCAAGGAAG | 4800 |
| TGCCTTCATT CTGCTTTGTA CTAGGACACC AAAGACATCA AGTACTCATC ACCCACCCAT | 4860 |
| ATCATCAACA GCCTCTAAAG GCTCAGAGGG AATCTGCCTT GCAGCTCTAC TCTGCCCCAG | 4920 |
| GGCTTGTGGC CAGCCATTTC TCACAGAGAG CTGGCTGCCT TGAGGGCATT CACCTGGCAC | 4980 |
| CAGTTTCAGG GCCTCACCCA AGCTTTGCAG GGGAAAGCAC AGAGGGAGGA ATTACACTGA | 5040 |
| AAAAAATGCA AGCAAAGGTT GAGTACCCCC AGGTGCCCCT TAGGAAGGAA CCAGGTTTAA | 5100 |
| ATAGGCTCTA CCCTTACCTT TCCCAGCAGC AAGTTCAGGG GAAGAGGCCT ACTCTTAGCC | 5160 |
| CTGGCTAGTG TGACCCTCTT CCTGTCCTAA GACTTTGGTC CTACCACCTC TTGTTTCATC | 5220 |
| TTTCCTTTAC ATTGCTGGGG GTTACCGCAG GTGCCTACCC CAGGGCTTCA CCATATGGGC | 5280 |
| CATTAATAGC TCTACTAAAA CTGACTTCTA GATGTAGGTT TCATTATTGG GGGAGGGGGT | 5340 |
| TCTTATTGTT ATATTTTAAA TGGCCTTTTG ATTTTATTTA TTTTTATGTT TTGATTATTT | 5400 |
| TTTTCTTTTT TAACTAATAA GGCGAGAAGA GGGAAGTTGG AGAGGGAAAA GTTAGCCCAG | 5460 |
| AAGGAAAGCA TTTTCTGCAG ATCAGCCTGA ATCCACCGTG GCTAGGCATA TTCTTGCTCT | 5520 |
| TCTCGTGTTG CTCACAACTA CCTGCCTGGA TGAATTTAGG AAAGTTGCAG GATACAAGGT | 5580 |
| TAAAACACAA GATCAAATGA ACAATCCGAA AATGTTATTA AGAAAACAGT TCCGGCCGGG | 5640 |
| CATGGTGGCT CACGCCTGAA ATCCCAGCAC TTTGGGAGGC CGAGGCAGGT GGATCACGAG | 5700 |
| GTCAGGAGAT CAAGACCATC CTGGCTAACA CGGTGAAACC CTATCTCTAC TAAAAATACA | 5760 |
| AAAAATTAGC CAGGTGTGGT GGCACGCACC AGTAGTCCCA GCTACTCGGG AGGCTGAGGC | 5820 |
| AGGAGAATTG CTTGAACCTG GAAGGCAGAG ATTGCAGTGA GCTGAGACCA CACCACTGCA | 5880 |
| CTCCATCCTG GGCAACAGAG TGAGACTTTG TCTCAAAAAG AAAGAAAGAA AGAAAGAAAG | 5940 |
| AAAGAAAGAA AGAAAAGAAA GAAAGAAAGA AAGAAAGAAA ACAGTTCCAT TTACAATAGC | 6000 |
| ATC | |

Seq ID NO: 59 Protein sequence:
Protein Accession #: XP_050478

| | |
|---|---|
| 1 11 21 31 41 51 | |
| \| \| \| \| \| \| | |
| MENRPCSFQY VPVQLQGGAP WGFTLKGCLE HCEPLTVSKI EDCGKAALSQ KMRTGDELVN | 60 |
| INGTPLYGSR QEALILIKGS FRILKLIVRR RNAPVSRPHS WHVAKLLECC PEAATTMHFP | 120 |
| SEAFSLSWHS CCNTSDVCVQ WCPLSRHCST EKSSSIGSME SLEQPCQATY ESHLLPIDQN | 180 |
| MYPNQRDSAY SSFSASSNAS DCALSLRPEE PASTDCIMQG PGPTKAPSGR PNVAETSGGS | 240 |
| RRTNGGHLTP SSQMSSRPQE GYQSGPAKAV RGPPQPPVRR DSLQASRAQL LNCEQRRASE | 300 |
| PVVPLPQKEK LSLEPVLPAR NPNRFCCLSG HDQVTSEGHQ NCEFSQPPES SQQGSEHLLM | 360 |
| QASTKAVGSP KACDRASSVD SNPLNEASAE LAKASFGRPP HLIGPTGHRH SAPEQLLASH | 420 |
| LQHVHLDTRC SKGMELPPVQ DGHQWTLSPL HSSHKGKKSP CPPTGGTHDQ SSKERKTRQV | 480 |
| DDRSLVLGHQ SQSSPPHGEA DGHPSEKGFL DFNRTSRAAS ELANQQPSAS GSLVQQATDC | 540 |
| SSTTKAASGT EAGEECGSEP KECSRMCCRR SGGTRCRSIQ NRRKSERFAT NLRNEIQRRK | 600 |
| AQLQKSKGPL SQLCDTKEPV EETQEPPESP PLTASNTSLL SSCKKPPSPR DKLFNKSMML | 660 |
| RARSSECLSQ APESHESRTG LEGRISPGQR PGQSSLGLNT WWKAPDPSSS DPEKAHAHCG | 720 |
| VRCGHWRWSP EHNSQPLVAA ANEGPSNPGD NKELKASTAQ ACEDAILLPF APPREFFEES | 780 |
| SKSLSTSHLP GLTTHSNKTF TQRPKPIDQN FQPMSSSCRE LRRHPMDQSY HSADQPYHAT | 840 |
| DQSYHSMSPL QSETPTYSEC FASKCLENSM CCKPLHCGDF DYHRTCSYSC SVQGALVHDP | 900 |
| CIYCSGEICP ALLKRNMMPN CYNCRCHHHQ CIRCSVCYHN PQHSALEDSS LAPGNTWKPR | 960 |
| KLTVQEFPGD KWNPITGNRK TSQSCREMAH SKTSFSWATP FHPCLENPAL DLSSYRAISS | 1020 |
| LDLLGDFKHA LKKSEETSVY EEGSSLASMP HFLRSRAFSE SHISLAPQST RAWGQERREL | 1080 |
| FSKGDETQSD LLGARKKAFP PPRFPPPNWE KYRLFRAAQQ QKQQQQQQKQ QEEEEEEEEE | 1140 |
| EEEEEEEEEE EAEEEEEELP PQYFSSETSC SCALNPEEVL EQPQPLSFGH LEGSRQGSQS | 1200 |
| VPAEQESFAL HSSDFLPPIR GHLCSQPEQA QPPCYYGIGG LWRTSGQEAT ESAKQEFQHF | 1260 |
| SPPSGAPGIP TSYSAYYNIS VAKAELLNKL KDQPEMAETG LGEEEVDHEL AQKKIQLIES | 1320 |
| ISRKLSVLRE AQRGLLEDIN ANSALGEEVE ANLKAVCKSN EFEKYHLFVG DLDKVVNLLL | 1380 |
| SLSGRLARVE NALNSIDSEA NQEKLVLIEK KQQLTGQLAD AKELKEHVDR REKLVFGMVS | 1440 |
| RYLPQDQLQD YQHFVKNKSA LIIEQRELEE KIKLGEEQLK CLRESLLLGP SHE | |

Seq ID NO: 60 Nucleotide sequence:
Nucleic Acid Accession #: NM_014705
Coding sequence: 192 . . . 2489 (underlined sequences correspond to
start and stop codons)

| | |
|---|---|
| 1 11 21 31 41 51 | |
| \| \| \| \| \| \| | |
| GGGAGAAGCT AGGAAAAAAT GTCTTTGAGC TGTGAGATGC TTGTATATTT TGAAAATATG | 60 |
| ATTATATGCA TGTGTTTGTA TTTTATGACT TGGATAATCT GAAAATCAAT TTGCTTTGTC | 120 |
| AATGCTTCCT GGATTAGAAT TCCACTATTT GGTCCCTATC CTAGTCTACT AAAGAAAATT | 180 |
| GAGCGGGAAA CATGGCGGGA AAGTGGCGTT TCATTAATTG CTACTGTAAC TCGTCTAATG | 240 |
| GAGAGGTTGT TAGATTACAG AACTTCTATA AGACTGAACT GAACAAGGAG GAGATGTATA | 300 |
| TACGCTACAT TCACAAACTC TATGATCTGC ATCTCAAAGC ACAGAACTTT ACAGAAGCTG | 360 |
| CATATACCCT CCTCTTATAT GACGAGCTAC TGGAATGGTC TGATCGGCCC CTCAGGGAGT | 420 |
| TCCTGACCTA CCCCATGCAA ACAGAATGGC AGCGCAAAGA GCACCTGCAC CTCACCATCA | 480 |
| TCCAGAACTT TGACAGAGGC AAATGTTGGG AGAATGGCAT TATCTTGTGC CGGAAGATTG | 540 |
| CAGAGCAGTA TGAGAGTTAT TATGACTACA GAAACCTGAG CAAGATGCGG ATGATGGAAG | 600 |
| CCTCTTTGTA TGACAAAATT ATGGACCAGC AACGTCTTGA ACCAGAGTTC TTCAGAGTTG | 660 |
| GATTTTATGG AAAAAAATTT CCATTTTTCT TAAGAAATAA GGAGTTTGTG TGTCGAGGGC | 720 |
| ATGACTACGA GAGGCTGGAA GCCTTCCAAC AGAGAATGCT GAACGAGTTC CCCCATGCCA | 780 |
| TCGCCATGCA GCACGCCAAC CAGCCCGATG AGACCATCTT CCAGGCAGAA GCTCAGTATT | 840 |
| TGCAGATATA TGCTGTGACT CCCATTCCAG AGAGCCAGGA GGTCCTGCAG AGAGAGGGTG | 900 |
| TTCCGGACAA CATCAAAAGC TTCTATAAAG TGAATCACAT CTGGAAATTC CGCTATGACC | 960 |
| GACCATTTCA CAAAGGCACA AAAGATAAAG AGAATGAATT CAAGAGTCTC TGGGTGGAGA | 1020 |
| GAACGTCATT ATACTTGGTG CAGAGTTTGC CTGGCATCTC TCGCTGGTTT GAAGTGGAAA | 1080 |
| AGCGTGAAGT GGTAGAAATG AGTCCTCTGG AAAATGCAAT TGAAGTGCTA GAAAATAAGA | 1140 |
| ATCAGCAGCT GAAGACTCTG ATTAGTCAGT GTCAGACAAG ACAGATGCAG AATATTAATC | 1200 |
| CCCTGACTAT GTGCCTGAAT GGAGTTATAG ATGCTGCAGT TAATGGTGGC GTTTCCAGGT | 1260 |
| ATCAAGAGGC ATTCTTTGTC AAAGAATATA TCTTAAGTCA CCCTGAAGAT GGGGAGAAAA | 1320 |
| TTGCACGATT AAGAGAGCTG ATGCTTGAGC AGGCACAGAT TCTGGAATTT GGTTTGGCCG | 1380 |
| TGCATGAGAA GTTTGTACCT CAAGATATGA GACCCCTTCA CAAAAAGCTG GTTGACCAAT | 1440 |
| TCTTTGTGAT GAAGTCGAGC TTAGGGATAC AGGAGTTCTC TGCTTGTATG CAAGCCAGTC | 1500 |
| CTGTCCATTT TCCTAATGGA AGCCCTCGTG TGTGTAGAAA CTCAGCACCT GCTTCTGTGA | 1560 |
| GCCCAGATGG TACCAGGGTA ATTCCTAGAC GCAGCCCGTT AAGTTACCCA GCTGTCAACC | 1620 |
| GATATTCTTC CTCCTCACTG TCCTCACAAG CTTCTGCTGA AGTAAGCAAT ATTACAGGGC | 1680 |
| AATCAGAAAG CTCTGATGAA GTCTTTAACA TGCAGCCAAG TCCATCTACC TCAAGCTTGA | 1740 |
| GTTCTACTCA CTCGGCTTCA CCTAATGTGA CAAGTTCTGC TCCATCGAGT GCCAGAGCTT | 1800 |
| CTCCTTTGTT GTCTGACAAA CACAAACATT CCCGAGAAAA CTCTTGCCTG TCACCAAGAG | 1860 |
| AGAGACCATG CAGTGCCATC TATCCAACAC CTGTGGAGCC TTCGCAGAGG ATGCTGTTTA | 1920 |
| ATCATATTGG AGACGGGGCC TTGCCACGCA GTGACCCAAA TCTCTCTGCA CCTGAAAAAG | 1980 |
| CTTCACCAGC AAGACACACG ACATCAGTAT CCCCCTCGCC TGCCGGGCGA TCTCCATTGA | 2040 |
| AGGGCTCTGT GCAGTCTTTC ACCCCCTCTC CAGTGGAGTA CCACTCGCCA GGACTCATCT | 2100 |
| CCAACTCCCC TGTCTTGTCG GGCAGCTACA GCAGTGGGAT TTCTTCTCTC AGCCGGTGCA | 2160 |
| GCACGTCGGA AACCTCAGGC TTTGAAAATC AGGTGAATGA ACAGTCGGCC CCCCTGCCGG | 2220 |
| TGCCAGTGCC GGTGCCCCTG CCGAGCTACG CCCGCGAGCA GCCAGTGCGC AAGGAGAGCA | 2280 |
| AGACTCCGCC CCCGTACAGC GTCTACGAGC GGACTCTGCG GCGCCCCGTC CCGCTACCTC | 2340 |
| ACAGCCTCTC CATCCCCGTC ACGTCGGAGC CGCCCGCGCT GCCCCCCAAG CCTCTGGCAG | 2400 |
| CGCGATCCAG CCACCTGGAG AATGGGGCCC GGAGGACTGA CCCCGGCCCG CGGCCCAGGC | 2460 |
| CCCTGCCCCG CAAGGTCTCT CAGTTATAAG TCACTTTTCT ATGTACCTGC GATGCATTCT | 2520 |
| TTGCCCGTTT ACAAAATAAG AAGTATGATG AGAAGACATT TAGTGTAGGC ACTTTAATAA | 2580 |
| CTTACTCAGC TCCTTCGATG AATGGAATTA AAACTTGCTT ATTAAATATC ATGTTGCACA | 2640 |
| ATATTAAAAG TTGCTGATCT AAAACGCCAG ATGTTAAATG AAGTATGGCT GAATTTCATT | 2700 |
| AAAACGTTTC TCATTTGGAA GTGGTAAATA GTGATAAAGA CTCCTTTTGT ACCTTTTTAT | 2760 |
| GTTCACTTTT TTTTATATAG TTTAATCTTA AAACCAATAC GATATTGTCA AACGATACAA | 2820 |
| TGTGTGACAA TGTTGTATCG TTTTTACTGA ATACTTGATA CTTGGAGAAA GCTTATTAAG | 2880 |
| TCAGTGCACA TCCTAACACA GTGGTCCTTA TTTTAGAAGA CTTCTGTAAA TAAGGCAAGG | 2940 |
| TTTATCAGTG CAGATCATCA GAATTAAAGT TCAAGCAGGC GAGCAAGACA GTATACTTAA | 3000 |
| GGGGTTGCAA AGCTTGGGAC TGGAAATTGT TTTGTTCTTG AAACAAAATA CTTCTTTAAG | 3060 |
| GTTGCTTTTG CTGTTTGACT GCTGTCTACA TTCGTAAAAT TCTATTTTGT GAATTGGTAG | 3120 |
| CTAAATCCCT TACTACCCTG ACACCGTGGT ATCTACTGTA TTTCTTTTCA AGGTGCAATT | 3180 |
| TGCTTCAGAG TTCCAATCAG CTAGATTAAG CAAGAGGCTC CAGAAGAAAT GTTTACTTGA | 3240 |
| ATTTTGCGCT TCCTTTCTTG ATACTTTCCT ATATAAAATT TGTCATTGAA CAAGAGCAAA | 3300 |
| TGCTGAAGTA TTAATGAGGC ACAAATGACT GTGCCCCATT AGCAAGAATT CAGGAATCAA | 3360 |
| TACAGACAGT ATTAAATTAA TAGCTTAAGT GAAGAAAAAA AAAAACTTAG TGAAAATGTA | 3420 |
| TTAGCACGAT TAAATGGCAA AAGGACTTAT AAAAGGCAAG GGCATTAACT TTCAGTCCTG | 3480 |
| CACAAAATAA AAAATTCCTC ACGACTCTCC ACTTTTACCA GTGGAGTTTG TCTTAGCTGA | 3540 |
| CCTGTCGTCT TTCTCTTGAA GGAGGATTGC TGTAGACTTC TCTAGCTTGA ATATTGCAAC | 3600 |
| ATAGCATCTT AGGTCTAGAT AGGGATGCTA ATGCCAGTTG TAGAAGTGTG AAAAAAGCAC | 3660 |
| CTTGTATGTA GTAATGTATT TTATATCTTT GTTTTTTCTT TTACTGACTG TTTATAACAC | 3720 |
| TCAATTGACA ATAGATATGA ACTGTATTTT AAATCATACT GTTAAATATT TTCCCTCTTT | 3780 |
| TGTTGGGAAG CTCATTTTAG TTTAACCATG TTTGTTTTGT TGGTAGCTTA CCTGGAAGGC | 3840 |
| AGTGACCACT TTTTTATATT CTCTTAATGA AACCATTCAG CAGGTATATG CTGTTGAGGC | 3900 |
| TGGTTATAGA GGTTTTCTAT AATAAATGTT CAAGTATTTT TGTATATAAC TGGTTAATTT | 3960 |
| TAATAAGAGA TACCATTATG TGTAAAAAAA AGTAAAAATA AACGCAAACA GTTGTTGATG | 4020 |
| CAGTATGATT GTTATAATTA TGCCAAATAC TTTACGTATG GAAAAAGAAT ATTTGTACAT | 4080 |
| ATGTGCTTTT AACAATTCTG CCATATTGAC TTTACAATTT TGAATGTCGG AAAAATTAAT | 4140 |
| ATATGTTAAA TATTTATGTT TAGTGAAAGT GTTCATAATT GAGAAAAGGA ACATATGCAT | 4200 |
| TTTAGCTTTG TATCTTGCAA GTTTTGCAGT CAGAAATTTT TTGAACTAGC TTTTGCTTTT | 4260 |
| GATAACACTT CGTGTTTGTA ACCACATTCA TATATATATA CATATATATG TGAAGCTCCA | 4320 |
| TATTTCTGTT GCTTTAAAGA AGTAAAACCT TCCATTTAAA TAAGATGACA TGCATAAGAT | 4380 |
| AACAAAGCTT CCTTGATTTC CTTTTCCTGT GTAATTTAAT AGATTTGTTG ACTAGTGCTT | 4440 |
| GGCCACATTA TAAATCAGTG TTATTTGCTC TTGGAGCCAT TTTTTAAAAA AAATTTTGGC | 4500 |
| AGTGAGCAGT TGAATTTATC TTGAATTTAT CATGTGTGTG TATTTCTGAA GCAGCTACAT | 4560 |
| AGCAGAACAT TTTAAGAGAT TCTGTTAGCC CACATGTTCA TGTTGGTTGC TGCTGAATGG | 4620 |
| TAAATATTAA ATAAAATTAC CAGATTAATC TT | |

Seq ID NO: 61 Protein sequence:
Protein Accession #: NP_055520

| | |
|---|---|
| 1 11 21 31 41 51 | |
| \| \| \| \| \| \| | |
| NAGKWRFINC YCNSSNGEVV RLQNFYKTEL NKEEMYIRYI HKLYGLHLKA QNFTEAAYTL | 60 |
| LLYDELLEWS DRPLREFLTY PMQTEWQRKE HTHLTIIQNF DRGKCWENGI ILCRKIAEQY | 120 |
| ESYYDYRNLS KMRMMEASLY DKIMDQQRLE PEFFRVGFYG KKFPFFLRNK EFVCRGHDYE | 180 |
| RLEAFQQRML NEFPHAIANQ HANQPDETIF QAEAQYLQTY AVTPIPESQS VLQREGVPDN | 240 |
| IKSFYKVNHI WKFRYDRPFH KGTKDKENEF KSLWVERTSL YLVQSLPGIS RWFEVEKREV | 300 |
| VEMSPLENAI EVLENKNQQL KTLISQCQTR QMQNINPLTM CLNGVIDAAV NGGVSRYQEA | 360 |
| FFVKEYILSH PEDGEKIARL RELMLEQAQI LEFGLAVHEK FVPQDMRPLH KKLVDQFFVM | 420 |
| KSSLGIQEFS ACMQASPVHF PNGSPRVCRN SAPASVSPDG TRVIPRRSPL SYPAVNRYSS | 480 |
| SSLSSQASAE VSNITGQSES SDEVFNMQPS PSTSSLSSTH SASPNVTSSA PSSARASPLL | 540 |
| SDKHKHSREN SCLSPRERPC SAIYPTPVEP SQRMLFNHIG DGALPRSDPN LSAPEKASPA | 600 |
| RHTTSVSPSP AGRSPLKGSV QSFTPSPVEY HSPGLISNSP VLSGSYSSGI SSLSRCSTSE | 660 |
| TSGFENQVNE QSAPLPVPVP VPVPSYGGEE PVRKESKTPP PYSVYERTLR RPVPLPHSLS | 720 |
| IPVTSEPPAL PPKPLAARSS HLENGARRTD PGPRPRPLPR KVSQL | |

Seq ID NO: 62 Nucleotide sequence:
Nucleic Acid Accession #: fgenesh prediction
Coding sequence: 1 . . . 2561 (underlined sequences correspond to
start and stop codons)

| | |
|---|---|
| 1 11 21 31 41 51 | |
| \| \| \| \| \| \| | |
| ATGGACCGAG GCCAGGGTAA GAGGGGCCGC GACGCCCGCA CTTGTTGCGG CGCCGGGCGG | 60 |
| GAAAGGGAGA CTGGACGATC TGAAGCCGGA GAGGAGGAGG GAGAGAGGCG GGCGGTGGGG | 120 |
| CGGGGGCTGA GGAACGCTCG GAGGGGACTG GGAGACGCGG CGCTTATGCA AAGGTGCCTT | 180 |
| CGGCTGCCGG GACAACCCGC CAGCAACCAG GTACAGCTCT CAGAGGTTCC ACAGAGGAAG | 240 |
| CTCAGGGTCC CTGAATCTCC CAGTGTGGCA GAGAAAGTGA AACTTGGTCA CCGATGCCTG | 300 |
| GAACTGCTGG AGCAGCTGCT CCCAGAGCTC ACCGGGCTGC TCAGCCTCCT GGACCACGAG | 360 |
| TACCTCAGCG ATACCACCCT GGAAAAGAAG ATGGCCGTGG CCTCCATCCT GCAGAGCCTG | 420 |
| CAGCCCCTTC CAGCAAAGGA GGTCTCCTAC CTGTATGTGA ACACAGCAGA CCTCCACTCG | 480 |
| GGGCCCAGCT TCGTGGAATC CCTCTTTGAA GAATTTGACT GTGACCTGAG TGACCTTCGG | 540 |
| GACATGCCAG AGGATGATGG GGAGCCCAGC AAAGGAGCCA GCCCTGAGCT AGCCAAGAGC | 600 |
| CCACGCCTGA GAAACGCGGC CGACCTGCCT CCACCGCTCC CCAACAAGCC TCCCCCTGAG | 660 |
| GACTACTATG AAGAGGCCCT TCCTCTGGGA CCCGGCAAGT CGCCTGAGTA CATCAGCTCC | 720 |
| CACAATGGCT GCAGCCCCTC ACACTCGATT GTGGATGGCT ACTATGAGGA CGCAGACAGC | 780 |
| AGCTACCCTG CAACCAGGGT GAACGGCGAG CTTAAGAGCT CCTATAATGA CTCTGACGCA | 840 |
| ATGAGCAGCT CCTATGAGTC CTACGATGAA GAGGAGGAGG AAGGGAAGAG CCCGCAGCCC | 900 |
| CGACACCAGT GGCCCTCAGA GGAGGCCTCC ATGCACCTGG TGAGGGAATG CAGGATATGT | 960 |
| GCCTTCCTGC TGCGGAAAAA GCGTTTCGGG CAGTGGGCCA AGCAGCTGAC GGTCATCAGG | 1020 |
| GAGGACCAGC TCCTGTGTTA CAAAAGCTCC AAGGATCGGC AGCCACATCT GAGGTTGGCA | 1080 |
| CTGGATACCT GCAGCATCAT CTACGTGCCC AAGGACAGCC GGCACAAGAG GCACGAGCTG | 1140 |
| CGTTTCACCC AGGGGGCTAC CGAGGTCTTG GTGCTGGCAC TGCAGAGCCG AGAGCAGGCC | 1200 |
| GAGGAGTGGC TGAAGGTCAT CCGAGAAGTG AGCAAGCCAG TTGGGGGAGC TGAGGGAGTG | 1260 |
| GAGGTCCCCA GATCCCCAGT CCTCCTGTGC AAGTTGGACC TGGACAAGAG GCTGTCCCAA | 1320 |
| GAGAAGCAGA CCTCAGATTC TGACAGCGTG GGTGTGGGTG ACAACTGTTC TACCCTTGGC | 1380 |
| CGCCGGGAGA CCTGTGATCA CGGCAAAGGG AAGAAGAGCA GCCTGGCAGA ACTGAAGGGC | 1440 |
| TCAATGAGCA GGGCTGCGGG CCGCAAGATC ACCCGTATCA TTGGCTTCTC CAAGAAGAAG | 1500 |
| ACACTGGCCG ATGACCTGCA GACGTCCTCC ACCGAGGAGG AGGTTCCCTG CTGTGGCTAC | 1560 |
| CTGAACGTGC TGGTGAACCA GGGCTGGAAG GAACGCTGGT GCCGCCTGAA GTGCAACACT | 1620 |
| CTGTATTTCC ACAAGGATCA CATGGACCTG CGAACCCATG TGAACGCCAT CGCCCTGCAA | 1680 |
| GGCTGTGAGG TGGCCCCGGG CTTTGGGCCC CGACACCCAT TTGCCTTCAG GATCCTGCGC | 1740 |
| AACCGGCAGG AGGTGGCCAT CTTGGAGGCA AGCTGTTCAG AGGACATGGG TCGCTGGCTC | 1800 |
| GGGCTGCTGC TGGTGGAGAT GGGCTCCAGA GTCACTCCGG AGGCGCTGCA CTATGACTAC | 1860 |
| GTGGATGTGG AGACCTTAAC CAGCATCGTC AGTGCTGGGC GCAACTCCTT CCTATATGCA | 1920 |
| AGATCCTGCC AGAATCAGTG GCCTGAGCCC CGAGTCTATG ATGATGTTCC TTATGAAAAG | 1980 |
| ATGCAGGACG AGGAGCCCGA GCGCCCCACA GGGGCCCAGG TGAAGCGTCA CGCCTCCTCC | 2040 |
| TGCAGTGAGA AGTCCCATCG TGTGGACCCG CAGGTCAAAG TCAAACGCCA CGCCTCCAGT | 2100 |
| GCCAATCAAT ACAAGTATGG CAAGAACCGA GCCGAGGAGG ATGCCCGGAG GTACTTGGTA | 2160 |
| GAAAAAGAGA AGCTGGAGAA AGAGAAAGAG ACGATTCGGA CAGAGCTGAT AGCACTGAGA | 2220 |
| CAGGAGAAGA GGGAACTGAA GGAAGCCATT CGGAGCAGCC CAGGAGCAAA ATTAAAGGCT | 2280 |
| CTGGAAGAAG CCGTGGCCAC CCTGGAAGCT CAGTGTCGGG CAAAGGAGGA GCGCCGGATT | 2340 |
| GACCTGGAGC TGAAGCTGGT GGCTGTGAAG GAGCGCTTGC AGCAGTCCCT GGCAGGAGGG | 2400 |
| CCAGCCCTGG GGCTCTCCGT GAGCAGCAAG CCCAAGAGTG GGCAACTCTC TGAGGAAGAT | 2460 |
| ACGCTCACCT CCAATGGTGC TCTCTCAGAG AGAACTTCTC TGACCTCATC TACACCAGGG | 2520 |
| CTTCTCAACC CCAACACTAC TGACATTTTG GACCAGTAA | |

Seq ID NO: 63 protein sequence:
Protein Accession #: fgenesh prediction

| | |
|---|---|
| 1 11 21 31 41 51 | |
| \| \| \| \| \| \| | |
| MDRGQGKRGR DARTCCGAGR ERETGRSEAG EEECERRAVG RGLRNARRGL GDAALMQRCL | 60 |
| RLPGQPASNQ VQLSEVPQRK LRVPESPSVA EKVKLGHRCL ELLEQLLPEL TGLLSLLDHE | 120 |
| YLSDTTLEKK MAVASILQSL QPLPAKEVSY LYVNTADLHS GPSFVESLFE EFDCDLSDLR | 180 |
| DMPEDDGEPS KGASPELAKS PRLRNAADLP PPLPNKPPPE DYYEEALPLG PGKSPEYISS | 240 |
| HNGCSPSHSI VGGYTEDADS SYPATRVNGE LKSSYNDSDA MSSSYESYDE EEEEGKSPQP | 300 |
| RHQWPSEEAS MHLVRECRIC AFLLRKKRFG QWAKQLTVIR EDQLLCYKSS KDRQPHLRLA | 360 |
| LDTCSIIYVP KDSRHKRHEL RFTQGATEVL VLALQSREQA EEWLKVIREV SKPVGGAEGV | 420 |
| EVPRSPVLLC KLDLDKRLSQ EKQTSDSDSV GVGDNCSTLG RRETCDHGKG KKSSLAELKG | 480 |
| SMSRAAGRKI TRIIGFSKKK TLADDLQTSS TEEEVPCCGY LNVLVNQGWK ERWCRLKCNT | 540 |
| LYFHKDHMDL RTHVNAIALQ GCEVAPGPGP RHPFAFRILR NRQEVAILEA SCSEDMGRWL | 600 |
| GLLLVEMGSR VTPEALHYDY VDVETLTSIV SAGRNSFLYA RSCQNQWPEP RVYDDVPYEK | 660 |
| MQDEEPERPT GAQVKRHASS CSEKSHRVDP QVKVKRHASS ANQYKYGKNR AEEDARRYLV | 720 |
| EKEKLEKEKE TIRTELIALR QEKRELKEAI RSSPGAKLKA LEEAVATLEA QCRAKEERRI | 780 |
| DLELKLVAVK ERLQQSLAGG PALGLSVSSK PKSGQLSEED TLTSNGALSE RTSLTSSTPG | 840 |
| LLNPNTTDIL DQ | |

Seq ID NO: 64 Nucleotide sequence:
Nucleic Acid Accession #: NM_004126.1
Coding sequence: 108-129(underlined sequences correspond to start
and stop codons)

| | |
|---|---|
| 1 11 21 31 41 51 | |
| \| \| \| \| \| \| | |
| GGCACGAGCT CGTGCCGGCC TTCAGTTGTT TCGGGACGCG CCGAGCTTCG CCGCTCTTCC | 60 |
| AGCGGCTCCG CTGCCAGAGC TAGCCCGAGC CCGGTTCTGG GGCGAAAATG CCTGCCCTTC | 120 |
| ACATCGAAGA TTTGCCAGAG AAGGAAAAAC TGAAAATGGA AGTTGAGCAG CTTCGCAAAG | 180 |
| AAGTGAAGTT GCAGAGACAA CAAGTGTCTA AATGTTCTGA AGAAATAAAG AACTATATTG | 240 |
| AAGAACGTTC TGGAGAGGAT CCTCTAGTAA AGGGAATTCC AGAAGACAAG AACCCCTTTA | 300 |
| AAGAAAAAGG CAGCTGTGTT ATTTCATAAA TAACTTGGGA GAAACTGCAT CCTAAGTGGA | 360 |
| AGAACTAGTT TGTTTTAGTT TTCCCAGATA AAACCAACAT GCTTTTTAAG GAAGGAAGAA | 420 |
| TGAAATTAAA AGGAGACTTT CTTAAGCACC ATATAGATAG GGTTATGTAT AAAAGCATAT | 480 |
| GTGCTACTCA TCTTTGCTCA CTATGCAGTC TTTTTTAAGA GAGCAGAGAG TATCAGATGT | 540 |
| ACAATTATGG AAATAAGAAC ATTACTTGAG CATGACACTT CTTTCAGTAT ATTGCTTGAT | 600 |
| GCTTCAAATA AAGTTTTGTC TT | |

Seq ID NO: 65 Protein sequence:
Protein Accession #: NP_004117

| | |
|---|---|
| 1 11 21 31 41 51 | |
| \| \| \| \| \| \| | |
| MPALHIEDLP EKEKLKMEVE QLRKEVKLQR QQVSKCSEEI KNYIEERSGE DPLVKGIPED | 60 |
| KNPFKEKGSC VIS | |

Seq ID NO: 66 Nucleotide sequence:
Nucleic Acid Accession #: NM_003842.1
Coding sequence: 1-1236 (underlined sequences correspond to start
and stop codons)

| | |
|---|---|
| 1 11 21 31 41 51 | |
| \| \| \| \| \| \| | |
| ATGGAACAAC GGGGACAGAA CGCCCCGGCC GCTTCGGGGG CCCGGAAAAG GCACGGCCCA | 60 |
| GGACCCAGGG AGGCGCGGGG AGCCAGGCCT GGGCCCCGGG TCCCCAAGAC CCTTGTGCTC | 120 |
| GTTGTCGCCG CGGTCCTGCT GTTGGTCTCA GCTCAGTCTG CTCTGATCAC CCAACAAGAC | 180 |
| CTAGCTCCCC AGCAGAGAGC GGCCCCACAA CAAAAGAGGT CCAGCCCCTC AGAGGGATTG | 240 |
| TGTCCACCTG GACACCATAT CTCAGAAGAC GGTAGAGATT GCATCTCCTG CAAATATGGA | 300 |
| CAGGACTATA GCACTCACTG GAATGACCTC CTTTTCTGCT TGCGCTGCAC CAGGTGTGAT | 360 |
| TCAGGTGAAG TGGAGCTAAG TCCCTGCACC ACGACCAGAA ACACAGTGTG TCAGTGCGAA | 420 |
| GAAGGCACCT TCCGGGAAGA AGATTCTCCT GAGATGTGCC GGAAGTGCCG CACAGGGTGT | 480 |
| CCCAGAGGGA TGGTCAAGGT CGGTGATTGT ACACCCTGGA GTGACATCGA ATGTGTCCAC | 540 |
| AAAGAATCAG GCATCATCAT AGGAGTCACA GTTGCAGCCG TAGTCTTGAT TGTGGCTGTG | 600 |
| TTTGTTTGCA AGTCTTTACT GTGGAAGAAA GTCCTTCCTT ACCTGAAAGG CATCTGCTCA | 660 |
| GGTGGTGGTG GGGACCCTGA GCGTGTGGAC AGAAGCTCAC AACGACCTGG GGCTGAGGAC | 720 |
| AATGTCCTCA ATGAGATCGT GAGTATCTTG CAGCCCACCC AGGTCCCTGA GCAGGAAATG | 780 |
| GAAGTCCAGG AGCCAGCAGA GCCAACAGGT GTCAACATGT TGTCCCCCGG GGAGTCAGAG | 840 |
| CATCTGCTGG AACCGGCAGA AGCTGAAAGG TCTCAGAGGA GGAGGCTGCT GGTTCCAGCA | 900 |
| AATGAAGGTG ATCCCACTGA GACTCTGAGA CAGTGCTTCG ATGACTTTGC AGACTTGGTG | 960 |
| CCCTTTGACT CCTGGGAGCC GCTCATGAGG AAGTTGGGCC TCATGGACAA TGAGATAAAG | 1020 |
| GTGGCTAAAG CTGAGGCAGC GGGCCACAGG GACACCTTGT ACACGATGCT GATAAAGTGG | 1080 |
| GTCAACAAAA CCGGGCGAGA TGCCTCTGTC CACACCCTGC TGGATGCCTT GGAGACGCTG | 1140 |
| GGAGAGAGAC TTGCCAAGCA GAAGATTGAG GACCACTTGT TGAGCTCTGG AAAGTTCATG | 1200 |
| TATCTAGAAG GTAATGCAGA CTCTGCCATG TCCTAA | |

Seq ID NO: 67 Protein sequence:
Protein Accession #: NP_003833.1

| | |
|---|---|
| 1 11 21 31 41 51 | |
| \| \| \| \| \| \| | |
| MEQRGQNAPA ASGARKRHGP GPREARGARP GPRVPKTLVL VVAAVLLLVS AESALITQQD | 60 |
| LAPQQRAAPQ QKRSSPSEGL CPPGHHISED GRDCISCKYG QDYSTNWNDL LFCLRCTRCD | 120 |
| SGEVELSPCT TTRNTVCQCE EGTFREEDSP EMCRKCRTGC PRGMVKVGDC TPWSDIECVH | 180 |
| KESGIIIGVT VAAVVLIVAV FVCKSLLWKK VLPYLKGICS GGGGDPERVD RSSQRPGAED | 240 |
| NVLNEIVSIL QPTQVPEQEM EVQEPAEPTG VNMLSPGESE NLLEPAEAER SQRRRLLVPA | 300 |
| NEGDPTETLR QCFGDFADLV PFDSWEPLMR KLGLMDNEIK VAKAEAAGHR DTLYTMLIKW | 360 |
| VNKTGRDASV HTLLDALETL GERLAKQKIE DHLLSSGKFM YLEGNADSAM S | |

Seq ID NO: 68 Nucleotide sequence:
Nucleic Acid Accession #: FGENESH predicted ORF
Coding sequence: 361-2220 (underlined sequences correspond to start
and stop codons)

| | |
|---|---|
| 1 11 21 31 41 51 | |
| \| \| \| \| \| \| | |
| GGCACCATCT GCTCCCTGCC CTGCCCAGAG GGCTTTCACG GACCCAACTG CTCCCAGGAA | 60 |
| TGTCGCTGCC ACAACGGCGG CCTCTGTGAC CGATTCACTG GGCAGTGCCG CTGCGCTCCG | 120 |
| GGTTACACTG GGGATCGGTG CCGGGAGGAG TGCCCGGTGG GCCGCTTTGG GCAGGACTGT | 180 |
| GCTGAGACGT GCGACTGCGC CCCGGACGCC CGTTGCTTCC CGGCCAACGG CGCATGTCTG | 240 |
| TGCGAACACG GCTTCACTGG GGACCGCTGC ACGGATCGCC TCTGCCCCGA CGGCTTCTAC | 300 |
| GGTCTCAGCT GCCAGGCCCC CTGCACCTGC GACCGGGAGC ACAGCCTCAG CTGCCACCCG | 360 |
| ATGAACGGGG AGTGCTCCTG CCTGCCGGGC TGGGCGGGCC TCCACTGCAA CGAGAGCTGC | 420 |
| CCGCAGGACA CGCATGGGCC AGGGTGCCAG GAGCACTGTC TCTGCCTGCA CGGTGGCGTC | 480 |
| TGCCAGGCTA CCAGCGGCCT CTGTCAGTGC GCGCCGGGTT ACACGGGCCC TCACTGTGCT | 540 |
| AGTCTTTGTC CTCCTGACAC CTACGGTGTC AACTGTTCTG CACGCTGCTC ATGTGAAAAT | 600 |
| GCCATCGCCT GCTCACCCAT CGACGGCGAG TGCGTCTGCA AGGAAGGTTG GCAGCGTGGT | 660 |
| AACTGCTCTG TGCCCTGCCC ACCCGGAACC TGGGGCTTCA GTTGCAATGC CAGCTGCCAG | 720 |
| TGTGCCCATG AGGCAGTCTG CAGCCCCCAA ACTGGAGCCT GTACCTGCAC CCCTGGGTGG | 780 |
| CATGGGGCCC ACTGCCAGCT GCCCTGTCCG AAGGGGCAGT TTGGAGAAGG TTGTGCCAGT | 840 |
| CGCTGTGACT GTGACCACTC TGATGGCTGT GACCCTGTTC ATGGACGCTG TCAGTGCCAG | 900 |
| GCTGGCTGGA TGGGTGCCCG CTGCCACCTG TCCTGCCCTG AGGGCTTATG GGGAGTCAAC | 960 |
| TGTAGCAACA CCTGCACCTG CAAGAATGGG GGCACCTGTC TCCCTGAGAA TGGCAACTGC | 1020 |
| GTGTGTGCAC CCGGATTCCG GGGCCCCTCC TGCCAGAGAT CCTGTCAGCC TGGCCGCTAT | 1080 |
| GGCAAACGCT GTGTGCCCTG CAAGTGCGCT AACCACTCCT TCTGCCACCC CTCGAACGGG | 1140 |
| ACCTGCTACT GCCTGGCTGG CTGGACAGGC CCCGACTGCT CCCAGCGCTG CCCTCTGGGG | 1200 |
| ACATTTGGTG CTAACTGCTC CCAGCCATGC CAGTGTGGTC CTGGAGAAAA GTGCCACCCA | 1260 |
| GAGACTGGGG CCTGTGTATG TCCCCCAGGG CACAGTGGTG CACCTTGCAG GATTGGAATC | 1320 |
| CAGGAGCCCT TTACTGTGAT GCCGACCACT CCAGTAGCGT ATAACTCGCT GGGTGCAGTG | 1380 |
| ATTGGCATTG CAGTGCTGGG GTCCCTTGTG GTAGCCCTGG TGGCACTGTT CATTGGCTAT | 1440 |
| CGGCACTGGC AAAAAGGCAA GGAGCACCAC CACCTGGCTG TGGCTTACAG CAGCGGGCGC | 1500 |
| CTGGACGGCT CCGAGTATGT CATGCCAGAT GTCCCTCCGA GCTACAGTCA CTACTACTCC | 1560 |
| AACCCCAGCT ACCACACCCT GTCGCAGTGC TCCCCAAACC CCCCACCCCC TAACAAGGTT | 1620 |
| CCAGGCCCGC TCTTTGCCAG CCTGCAGAAC CCTGAGCGGC CAGGTGGGGC CCAAGGGCAT | 1680 |
| GATAACCACA CCACCCTGCC TGCTGACTGG AAGCACCGCC GGGAGCCCCC TCCAGGGCCT | 1740 |
| CTGGACAGGG GGAGCAGCCG CCTGGACCGA AGCTACAGCT ATAGCTACAG CAATGGCCCA | 1800 |
| GCCCCATTCT ACAATAAAGG GCTCATCTCT GAAGAGGAGC TCGGGGCCAG TGTGGCTTCC | 1860 |
| CTGAGCAGTG AGAACCCATA TGCCACCATC CGGGACCTGC CCAGCTTGCC AGGGGGCCCC | 1920 |
| CGGGAGAGCA GCTACATGGA GATGAAAGGC CCTCCCTCAG GATCTCCCCC CAGGCAGCCT | 1980 |
| CCTCAGTTCT GGGACAGCCA GAGGCGGCGG CAACCCCAGC CACAGAGAGA CAGTGGCACC | 2040 |
| TACGAGCAGC CCAGCCCCCT GATCCAGGAC CGAGACTCTG TGGGCTCCCA GCCCCCTCTG | 2100 |
| CCTCCGGGCC TACCCCCCGG CCACTATGAC TCACCCAAGA ACAGCCACAT CCCTGGACAT | 2160 |
| TATGACTTGC CTCCAGTACG GCATCCCCCA TCACCTCCAC TTCGACGCCA GGACCGTTGA | |

Seq ID NO: 69 Protein sequence:
Protein Accession #: FGENESH prediction

| | |
|---|---|
| 1 11 21 31 41 51 | |
| \| \| \| \| \| \| | |
| GTICSLPCPE GFHGPNCSQE CRCHNGGLCD RFTGQCRCAP GYTGDRCREE CPVORFGQDC | 60 |
| AETCDCAPDA RCFPANGACL CEHGFTGDRC TDRLCPDGFY GLSCQAPCTC DREHSLSCHP | 120 |
| MNGECSCLPG WAGLHCNESC PQDTHGPGCQ EHCLCLHGGV CQATSGLCQC APGYTGPECA | 180 |
| SLCPPDTYGV NCSARCSCEN AIACSPIDCE CVCKEGWQRG NCSVPCPPGT WGFSCNASCQ | 240 |
| CAHEAVCSPQ TGACTCTPGW HGAHCQLPCP KGQFGEGCAS RCDCDHSDGC DPVHGRCQCQ | 300 |
| ASWMGARCHL SCPEGLWGVN CSNTCTCKNG GTCLPENGNC VCAPGFRGPS CQRSCQPGRY | 360 |
| GKRCVPCKCA NHSFCHPSNG TCYCLAGWTG PDCSQRCPLG TFGANCSQPC QCGPGEKCHP | 420 |
| ETGACVCPPG HSGAPCRIGI QEPFTVMPTT PVAYNSLGAV IGIAVLGSLV VALVALFIGY | 480 |
| RHWQKGKEHH HLAVAYSSGR LDGSEYVMPD VPPSYSHYYS NPSYHTLSQC SPNPPPPNKV | 540 |
| PGPLFASLQN PERPGGAQGH DNHTTLPADW KHRREPPPGP LDRGSSRLDR SYSYSYSNGP | 600 |
| GPFYNKGLIS EEELGASVAS LSSENPYATI RDLPSLPGGP RESSYMEMKG PPSGSPPRQP | 660 |
| PQFWDSQRRR QPQPQRDSGT YEQPSPLIHD RDSVGSQPPL PPGLPPGHYD SPKNSHIPGH | 720 |
| YDLPPVRHPP SPPLRRQDR | |

Seq ID NO: 70 Nucleotide sequence:
Nucleic Acid Accession #: NM_005458
Coding sequence: 1 . . . 2826 (underlined sequences correspond to
start and stop codons)

| | |
|---|---|
| 1 11 21 31 41 51 | |
| \| \| \| \| \| \| | |
| ATGGCTTCCC CGCGGAGGTC CGGGCAGCCA GGGCGGCCGC CGCCGCCGCC ACCGCCGCCC | 60 |
| GCGCGCCTGC TACTGCTACT GCTGCTGCCG CTGCTGCTGC CTCTGGCGCC CGGGGCCTGG | 120 |
| GGCTGGGCGC GGGGCGCCCC CCGGCCGCCG CCCAGCAGCC CGCCGCTCTC CATCATGGGC | 180 |
| CTCATGCCGC TCACCAAGGA GGTGGCCAAG GGCAGCATCG GGCGCGGTGT GCTCCCCGCC | 240 |
| GTGGAACTGG CCATCGAGCA GATCCGCAAC GAGTCACTCC TGCGCCCCTA CTTCCTCGAC | 300 |
| CTGCGGCTCT ATGACACGGA GTGCGACAAC GCAAAAGGGT TGAAAGCCTT CTACGATGCA | 360 |
| ATAAAATACG GGCCGAACCA CTTGATGGTG TTTGGAGGCG TCTGTCCATC CGTCACATCC | 420 |
| ATCATTGCAG AGTCCCTCCA AGGCTGGAAT CTGGTGCAGC TTTCTTTTGC TGCAACCACG | 480 |
| CCTGTTCTAG CCGATAAGAA AAAATACCCT TATTTCTTTC GGACCGTCCC ATCAGACAAT | 540 |
| GCGGTGAATC CAGCCATTCT GAAGTTGCTC AAGCACTACC AGTGGAAGCG CGTGGGCACG | 600 |
| CTGACGCAAG ACGTTCAGAG GTTCTCTGAG GTGCGGAATG ACCTGACTGG AGTTCTGTAT | 660 |
| GGCGAGGACA TTGAGATTTC AGACACCGAG AGCTTCTCCA ACGATCCCTG TACCAGTGTC | 720 |
| AAAAAGCTGA AGGGGAATGA TGTGCGGATC ATCCTTGGCC AGTTTGACCA GAATATGGCA | 780 |
| GCAAAAGTGT TCTGTTGTGC ATACGAGGAG AACATGTATG GTAGTAAATA TCAGTGGATC | 840 |
| ATTCCGGGCT GGTACGAGCC TTCTTGGTGG GAGCAGGTGC ACACGGAAGC CAACTCATCC | 900 |
| CGCTGCCTCC GGAAGAATCT GCTTGCTGCC ATGGAGGGCT ACATTGGCGT GGATTTCGAG | 960 |
| CCCCTGAGCT CCAAGCAGAT CAAGACCATC TCAGGAAAGA CTCCACAGCA GTATGAGAGA | 1020 |
| GAGTACAACA ACAAGCGGTC AGGCGTGGGG CCCAGCAAGT TCCAGGGGTA CGCCTACGAT | 1080 |
| GGCATCTGGG TCATCGCCAA GACACTGCAG AGGGCCATGG AGACACTGCA TGCCAGCAGC | 1140 |
| CGGCACCAGC GGATCCAGGA CTTCAACTAC ACGGACCACA CGCTGGGCAG GATCATCCTC | 1200 |
| AATGCCATGA ACGAGACCAA CTTCTTCGGG GTCACGGGTC AAGTTGTATT CCGGAATGGG | 1260 |
| GAGAGAATGG GGACCATTAA ATTTACTCAA TTTCAAGACA GCAGGGAGGT GAAGGTGGGA | 1320 |
| GAGTACAACG CTGTGGCCGA CACACTGGAG ATCATCAATG ACACCATCAG GTTCCAAGGA | 1380 |
| TCCGAACCAC CAAAAGACAA GACCATCATC CTGGAGCAGC TGCGGAAGAT CTCCCTACCT | 1440 |
| CTCTACAGCA TCCTCTCTGC CCTCACCATC CTCGGGATGA TCATGGCCAG TGCTTTTCTC | 1500 |
| TTCTTCAACA TCAAGAACCG GAATCAGAAG CTCATAAAGA TGTCGAGTCC ATACATGAAC | 1560 |
| AACCTTATCA TCCTTGGAGG GATGCTCTCC TATGCTTCCA TATTTCTCTT TGGCCTTGAT | 1620 |
| GGATCCTTTG TCTCTGAAAA GACCTTTGAA ACACTTTGCA CCGTCAGGAC CTGGATTCTC | 1680 |
| ACCGTCGGCT ACACGACCGC TTTTGGGGCC ATGTTTGCAA AGACCTGGAG AGTCCACGCC | 1740 |
| ATCTTCAAAA ATGTGAAAAT GAAGAAGAAG ATCATCAAGG ACCAGAAACT GCTTGTGATC | 1800 |
| GTGGGGGGCA TGCTGCTGAT CGACCTGTGT ATCCTGATCT GCTGGCAGGC TGTGGACCCC | 1860 |
| CTGCGAAGGA CAGTGGAGAA GTACAGCATG GAGCCGGACC CAGCAGGACG GGATATCTCC | 1920 |
| ATCCGCCCTC TCCTGGAGCA CTGTGAGAAC ACCCATATGA CCATCTGGCT TGGCATCGTC | 1980 |
| TATGCCTACA AGGGACTTCT CATGTTGTTC GGTTGTTTCT TAGCTTGGGA GACCCGCAAC | 2040 |
| GTCAGCATCC CCGCACTCAA CGACAGCAAG TACATCGGGA TGAGTGTCTA CAACGTGGGG | 2100 |
| ATCATGTGCA TCATCGGGGC CGCTGTCTCC TTCCTGACCC GGGACCAGCC CAATGTGCAG | 2160 |
| TTCTGCATCG TGGCTCTGGT CATCATCTTC TGCAGCACCA TCACCCTCTG CCTGGTATTC | 2220 |
| GTGCCGAAGC TCATCACCCT GAGAACAAAC CCAGATGCAG CAACGCAGAA CAGGCGATTC | 2280 |
| CAGTTCACTC AGAATCAGAA GAAAGAAGAT TCTAAAACGT CCACCTCGGT CACCAGTGTG | 2340 |
| AACCAAGCCA GCACATCCCG CCTGGAGGGC CTACAGTCAG AAAACCATCG CCTGCGAATG | 2400 |
| AAGATCACAG AGCTGGATAA AGACTTGGAA GAGGTCACCA TGCAGCTGCA GGACACACCA | 2460 |
| GAAAAGACCA CCTACATTAA ACAGAACCAC TACCAAGAGC TCAATGACAT CCTCAACCTG | 2520 |
| GGAAACTTCA CTGAGAGCAC AGATGGAGGA AAGGCCATTT TAAAAAATCA CCTCGATCAA | 2580 |
| AATCCCCAGC TACAGTGGAA CACAACAGAG CCCTCTCGAA CATGCAAAGA TCCTATAGAA | 2640 |
| GATATAAACT CTCCAGAACA CATCCAGCGT CGGCTGTCCC TCCAGCTCCC CATCCTCCAC | 2700 |
| CACGCCTACC TCCCATCCAT CGGAGGCGTG GACGCCAGCT GTGTCAGCCC CTGCGTCAGC | 2760 |
| CCCACCGCCA GCCCCCGCCA CAGACATGTG CCACCCTCCT TCCGAGTCAT GGTCTCGGGC | 2820 |
| CTGTAA | |

Seq ID NO: 71 protein sequence:
Protein Accession #: NP_005449

| | |
|---|---|
| 1 11 21 31 41 51 | |
| \| \| \| \| \| \| | |
| MASPRRSGQP GRPPPPPPPP ARLLLLLLLP LLLPLAPGAW GWARGAPRPP PSSPPLSIMG | 60 |
| LMPLTKEVAK GSTGRGVLPA VELATEQIRN ESLLRPYFLD LRLYDTECDN AKGLKAFYDA | 120 |
| IKYGPNHLMV FGGVCPSVTS IIAESLQGWN LVQLSFAATT PVLADKKKYP YFFRTVPSDN | 180 |
| AVNPAILKLL KHYQWKRVGT LTQDVQRFSE VRNDLTGVLY GEDIEISGTE SFSNDPCTSV | 240 |
| KKLKGNDVRI ILGQFDQNMA AKVFCCAYEE NMYGSKYQWI IPGWYEPSWW EQVHTEANSS | 300 |
| RCLRKNLLAA MEGYIGVDFE PLSSKQTKTI SGKTPQQYER EYNNKRSGVG PSKFHGYAYD | 360 |
| GIWVIAKTLQ RAMETLHASS RHQRIQDFNY TDHTLGRIIL NAMNETNFFG VTGQVVFRNG | 420 |
| ERMGTIKFTQ FQDSREVKVG EYNAVADTLE IINDTIRFQG SEPPKDKTII LEQLRKISLP | 480 |
| LYSILSALTI LGMIMASAFL FFNIKNRNQK LIKNSSPYMN NLIILGGMLS YASIELFGLD | 540 |
| GSFVSEKTFE TLCTVRTWIL TVGYTTAFGA MFAKTWRVHA IFKNVKMKKK IIKDQKLLVI | 600 |
| VGGMLLIDLC ILICWQAVDP LRRTVEKYSM EPGPAGRGIS IRPLLEHCEN THMTIWLGIV | 660 |
| YAYKGLLMLF GCFLAWETRN VSIPALNDSK YIGMSVYNVG IMCIIGAAVS FLTRDQPNVQ | 720 |
| FCIVALVIIF CSTITLCLVF VPKLITLRTN PDAATQNRRF QFTQNQKKED SKTSTSVTSV | 780 |
| NQASTSRLEG LQSENHRLRM KITELGKGLE EVTMQLQDTP EKTTYIKQNH YQELNDILNL | 840 |
| GNFTESTDGG KAILKNHLDQ NPQLQWNTTE PSRTCKDPIE DINSPEHIQR RLSLQLPILH | 900 |
| HAYLPSIGGV DASCVSPCVS PTASPRHRHV PPSFRVMVSG L | |

Seq ID NO: 72 Nucleotide sequence:
Nucleic Acid Accession #: NM_005795
Coding sequence: 522-1940 (underlined sequences correspond to start
and stop codons)

| | |
|---|---|
| 1 11 21 31 41 51 | |
| \| \| \| \| \| \| | |
| GCACGAGGGA ACAACCTCTC TCTCTSCAGC AGAGAGTGTC ACCTCCTGCT TTAGGACCAT | 60 |
| CAAGCTCTGC TAACTGAATC TCATCCTAAT TGCAGGATCA CATTGCAAAG CTTTCACTCT | 120 |
| TTCCCACCTT GCTTGTGGGT AAATCTCTTC TGCGGAATCT CAGAAAGTAA AGTTCCATCC | 180 |
| TGAGAATATT TCACAAAGAA TTTCCTTAAG AGCTGGACTG GGTCTTGACC CCTGGAATTT | 240 |
| AAGAAATTCT TAAAGACAAT GTCAAATATG ATCCAAGAGA AAATGTGATT TGAGTCTGGA | 300 |
| GACAATTGTG CATATCGTCT AATAATAAAA ACCCATACTA GCCTATAGAA AACAATATTT | 360 |
| GAATAATAAA AACCCATACT AGCCTATAGA AAACAATATT TGAAAGATTG CTACCACTAA | 420 |
| AAAGAAAACT ACTACAACTT GACAAGACTG CTGCAAACTT CAATTGGTCA CCACAACTTG | 480 |
| ACAAGGTTGC TATAAAACAA GATTGCTACA ACTTCTAGTT TATGTTATAC AGCATATTTC | 540 |
| ATTTGGGCTT AATGATGGAG AAAAAGTGTA CCCTGTATTT TCTGGTTCTC TTGCCTTTTT | 600 |
| TTATGATTCT TGTTACAGCA GAATTAGAAG AGAGTCCTGA GGACTCAATT CAGTTGGGAG | 660 |
| TTACTAGAAA TAAAATCATG ACAGCTCAAT ATGAATGTTA CCAAAAGATT ATGCAAGACC | 720 |
| CCATTCAACA AGCAGAAGGC GTTTACTGCA ACAGAACCTG GGATGGATGG CTCTGCTGGA | 780 |
| ACGATGTTGC AGCAGGAACT GAATCAATGC AGCTCTGCCC TGATTACTTT CAGGACTTTG | 840 |
| ATCCATCAGA AAAAGTTACA AAGATCTGTG ACCAAGATGG AAACTGGTTT AGACATCCAG | 900 |
| CAAGCAACAG AACATGGACA AATTATACCC AGTGTAATGT TAACACCCAC GAGAAAGTGA | 960 |
| AGACTGCACT AAATTTGTTT TACCTGACCA TAATTGGACA CGGATTGTCT ATTGCATCAC | 1020 |
| TGCTTATCTC GCTTGGCATA TTCTTTTATT TCAAGAGCCT AAGTTGCCAA AGGATTACCT | 1080 |
| TACACAAAAA TCTGTTCTTC TCATTTGTTT GTAACTCTGT TGTAACAATC ATTCACCTCA | 1140 |
| CTGCAGTGGC CAACAACCAG GCCTTAGTAG CCACAAATCC TGTTAGTTGC AAAGTGTCCC | 1200 |
| AGTTCATTCA TCTTTACCTG ATGGGCTGTA ATTACTTTTG GATGCTCTGT GAAGGCATTT | 1260 |
| ACCTACACAC ACTCATTGTG GTGGCCGTGT TTGCAGAGAA GCAACATTTA ATGTGGTATT | 1320 |
| ATTTTCTTGG CTGGGGATTT CCACTGATTC CTGCTTGTAT ACATGCCATT GCTAGAAGCT | 1380 |
| TATATTACAA TGACAATTGC TGGATCAGTT CTGATACCCA TCTCCTCTAC ATTATCCATG | 1440 |
| GCCCAATTTG TGCTGCTTTA CTGGTGAATC TTTTTTTCTT GTTAAATATT GTACGCGTTC | 1500 |
| TCATCACCAA GTTAAAAGTT ACACACCAAG CGGAATCCAA TCTGTACATG AAAGCTGTGA | 1560 |
| GAGCTACTCT TATCTTGGTG CCATTGCTTG GCATTGAATT TGTGCTGATT CCATGGCGAC | 1620 |
| CTGAAGGAAA GATTGCAGAG GAGGTATATG ACTACATGAT GCACATCCTT ATGCACTTCC | 1680 |
| AGGGTCTTTT GGTCTCTACC ATTTTCTGCT TCTTTAATGG AGAGGTTCAA GCAATTCTGA | 1740 |
| GAAGAAACTG GAATCAATAC AAAATCCAAT TTGGAAACAG CTTTTCCAAC TCAGAAGCTC | 1800 |
| TTCGTAGTGC GTCTTACACA GTGTCAACAA TCAGTGATGG TCCAGGTTAT ACTGATGACT | 1860 |
| GTCCTAGTGA ACACTTAAAT GGAAAAAGCA TCCATGATAT TGAAAATGTT CTCTTAAAAC | 1920 |
| CAGAAAATTT ATATAATTGA AAATAGAAGG ATGGTTGTCT CACTGTTTGG TGCTTCTCCT | 1980 |
| AACTCAAGGA CTTGGACCCA TGACTCTGTA GCCAGAAGAC TTCAATATTA AATGACTTTG | 2040 |
| GGGAATGTCA TAAAGAAGAG CCTTCACATG AAATTAGTAG TGTGTTGATA AGAGTGTAAC | 2100 |
| ATCCAGCTCT ATGTGGGAAA AAAGAAATCC TGGTTTGTAA TGTTTGTCAG TAAATACTCC | 2160 |
| CACTATGCCT GATGTGACGC TACTAACCTG ACATCACCAA GTGTGGAATT GGAGAAAAGC | 2220 |
| ACAATCAACT TTTCTGAGCT GGTGTAAGCC AGTTCCAGCA CACCATTGAT GAATTCAAAC | 2280 |
| AAATGGCTGT AAAACTAAAC ATACATGTTG GGCATGATTC TACCCTTATT CSCCCCAAGA | 2340 |
| GACCTAGCTA AGGTCTATAA ACATGAAGGG AAAATTAGCT TTTAGTTTTA AAACTCTTTA | 2400 |
| TCCCATCTTG ATTGGGGCAG TTGACTTTTT TTTTTTCCCA GAGTGCCGTA GTCCTTTTTG | 2460 |
| TAACTACCCT CTCAAATGGA CAATACCAGA AGTGAATTAT CCCTGCTGGC TTTCTTTTCT | 2520 |
| CTATGAAAAG CAACTGAGTA CAATTGTTAT GATCTACTCA TTTGCTGACA CATCAGTTAT | 2580 |
| ATCTTGTGGC ATATCCATTG TGGAAACTGG ATGAACAGGA TGTATAATAT GCAATCTTAC | 2640 |
| TTCTATATCA TTAGGAAAAC ATCTTAGTTG ATGCTACAAA ACACCTTGTC AACCTCTTCC | 2700 |
| TGTCTTACCA AACAGTGGGA GGGAATTCCT AGCTCTAAAT ATAAATTTTG TCCCTTCCAT | 2760 |
| TTCTACTGTA TAAACAAATT AGCAATCATT TTATATAAAG AAAATCAATG AAGGATTTCT | 2820 |
| TATTTTCTTG GAATTTTGTA AAAAGAAATT GTGAAAAATG AGCTTGTAAA TACTCCATTA | 2880 |
| TTTTATTTTA TAGTCTCAAA TCAAATACAT ACAACCTATG TAATTTTTAA AGCAAATATA | 2940 |
| TAATGCAACA ATGTGTGTAT GTTAATATCT GATACTGTAT CTGGGCTGAT TTTTTAAATA | 3000 |
| AAATAGAGTC TGGAATGCTA TATTTGGTAA ATATTTTAAA GACAACCAGA TGCCAGCATC | 3060 |
| AGAAGTCTGT TTGAGAACTA AGAGAACAGA AACATCTATC ATAAGATATA TTTATTTTAA | 3120 |
| AAACACAAGG TCACTATTTT ACTGAATATA TTTGTTTTGA TAACTCATAC CTTAATAATA | 3180 |
| GGTGTGTTTG ACATATTTCT TTTTTCATTT TGACAATGAA CTCACATTCT AATCCAGAAA | 3240 |
| TTTTAAACAA CTACTGTGAT AAATACCAAT CTGCTACTTT TATAGATTTT ACCCCATTAA | 3300 |
| AATATTACTT TACTGACTTT TACTATGTGA AGATATATAG CTTTGGAAAT GTCCCAGGCT | 3360 |
| ATTCAAGAAA TATAAAAAAC TAGAAGGATA CTATATATAC CATATACAAT GCTTTAATAT | 3420 |
| TTTAATAGAG CTACTGTATA TAATACAAAT TAGGGAAATA CTTGAATATA TCATTGAGAA | 3480 |
| AAAATTATTG TCAGATCTTA CTGAATTATT GTCAGACTTT ATTAAATAAA GATAGAAGAA | 3540 |
| AACCTTGCTA ATGAATTAAA GTGAAATTTG CATGGGATTC AGTTTCTCTA ATGTTATTTT | 3600 |
| CCGCTGAAAT CTCTAAAGAA CAAGAATGAC TTCAATTAGT AAAAGTCAAT TTTGGGAAAA | 3660 |
| GTCATGGGTA TCTGTTTTTT AAGTGTGTCA ATCTGATTAA AATGGATGAA ACAAATTACT | 3720 |
| TCAAATTGCT AACACAAATT ATCTAAATTC GTAACAATTA ACATATAGAA TGGTCTGGTC | 3840 |
| AGTACATTTA TAATTTATCT ATGCATGAAA AAGTATTGTT TTGTTTGAAA CATGAATTTC | 3900 |
| ATAGCAAGCT GCCATAGAAA GGA | |

Seq ID NO: 73 Protein sequence:
Protein Accession #: NM_005795

| | |
|---|---|
| 1 11 21 31 41 51 | |
| \| \| \| \| \| \| | |
| MLYSIFHLCL MMEKKCTLYF LVLLPFFMIL VTAELEESPE DSIQLCVTRN KIMTAQYECY | 60 |
| QKIMQGPIQQ AECVYCNRTW DCWLCWNDVA AGTESMQLCP DYFQDFDPSE KVTKICDQDG | 120 |
| NWFRHPASNR TWTNYTQCNV NTHEKVKTAL NLFYLTIICH GLSTASLLIS LGIFFYFKSL | 180 |
| SCQRITLHKN LFFSEVCNSV VTIIHLTAVA NNQALVATNP VSCKVSQFIH LYLMGCNYFW | 240 |
| MLCEGIYLHT LIVVAVFAEK QNLMWYYFLG WGFPLIPACI HAIARSLYYN DNCWISSDTH | 300 |
| LLYIIHGPIC AALLVNLFFL LNIVRVLITK LKVTHQAESN LYMKAVRATL ILVPLLCIEF | 360 |
| VLIPWRPECK IAEEVYDYIM HILMHFQGLL VSTIFCFFNG EVQAILRRNW NQYKIQFGNS | 420 |
| FSNSEALRSA SYTVSTISDG PGYSHDCPSE HLNGKSIHDI ENVLLKPENL YN | |

Seq ID NO: 74 Nucleotide sequence:
Nucleic Acid Accession #: NM_000450.1
Coding sequence: 117 . . . 1949 (underlined sequences correspond to
start and stop codons)

| | |
|---|---|
| 1 11 21 31 41 51 | |
| \| \| \| \| \| \| | |
| CCTGAGACAG AGGCAGCAGT GATACCCACC TGAGAGATCC TGTGTTTGAA CAACTGCTTC | 60 |
| CCAAAACGGA AAGTATTTCA AGCCTAAACC TTTGGGTGAA AAGAACTCTT GAAGTCATGA | 120 |
| TTGCTTCACA GTTTCTCTCA GCTCTCACTT TGGTGCTTCT CATTAAAGAG AGTGGAGCCT | 180 |
| GGTCTTACAA CACCTCCACG GAAGCTATGA CTTATGATGA GGCCAGTGCT TATTGTCAGC | 240 |
| AAAGGTACAC ACACCTGGTT GCAATTCAAA ACAAAGAAGA GATTGAGTAC CTAAACTCCA | 300 |
| TATTGAGCTA TTCACCAAGT TATTACTGGA TTGGAATCAG AAAAGTCAAC AATGTGTGGG | 360 |
| TCTGGGTAGG AACCCAGAAA CCTCTGACAG AAGAAGCCAA GAACTGGGCT CCAGGTGAAC | 420 |
| CCAACAATAG GCAAAAAGAT GAGGACTGCG TGGAGATCTA CATCAAGAGA GAAAAAGATG | 480 |
| TGGGCATGTG GAATGATGAG AGGTGCAGCA AGAAGAAGCT TGCCCTATGC TACACAGCTG | 540 |
| CCTGTACCAA TACATCCTGC AGTGGCCACG GTGAATGTGT AGAGACCATC AATAATTACA | 600 |
| CTTGCAAGTG TGACCCTGGC TTCAGTGGAC TCAAGTGTGA GCAAATTGTG AACTGTACAG | 660 |
| CCCTGGAATC CCCTGAGCAT GGAAGCCTGG TTTGCAGTCA CCCACTGGGA AACTTCAGCT | 720 |
| ACAATTCTTC CTGCTCTATC AGCTGTGATA GGGGTTACCT GCCAAGCAGC ATGGAGACCA | 780 |
| TGCAGTGTAT GTCCTCTGGA GAATGGAGTG CTCCTATTCC AGCCTGCAAT GTGGTTGAGT | 840 |
| GTGATGCTGT GACAAATCCA GCCAATGGGT TCGTGGAATG TTTCCAAAAC CCTGGAAGCT | 900 |
| TCCCATGGAA CACAACCTGT ACATTTGACT GTGAAGAAGG ATTTGAACTA ATGGGAGCCC | 960 |
| AGAGCCTTCA GTGTACCTCA TCTGGGAATT GGGACAACGA GAAGCCAACG TGTAAAGCTG | 1020 |
| TGACATGCAG GGCCGTCCGC CAGCCTCAGA ATGGCTCTGT GAGGTGCAGC CATTCCCCTG | 1080 |
| CTGGAGAGTT CACCTTCAAA TCATCCTGCA ACTTCACCTG TGAGGAAGGC TTCATGTTGC | 1140 |
| AGGGACCAGC CCAGGTTGAA TGCACCACTC AAGGGCAGTG GACACAGCAA ATCCCAGTTT | 1200 |
| GTGAAGCTTT CCAGTGCACA GCCTTGTCCA ACCCCGAGCG AGGCTACATG AATTGTCTTC | 1260 |
| CTAGTGCTTC TGGCAGTTTC CGTTATGGGT CCAGCTGTGA GTTCTCCTGT GAGCAGGGTT | 1320 |
| TTGTGTTGAA GGGATCCAAA AGGCTCCAAT GTGGCCCCAC AGGGGAGTGG GACAACGAGA | 1380 |
| AGCCCACATG TGAAGCTGTG AGATGCCATG CTGTCCACCA GCCCCCGAAG GGTTTGGTGA | 1440 |
| GGTGTGCTCA TTCCCCTATT GGAGAATTCA CCTACAAGTC CTCTTGTGCC TTCAGCTGTG | 1500 |
| AGGAGGGATT TGAATTATAT GGATCAACTC AACTTGAGTG CACATCTCAG GGACAATGGA | 1560 |
| CAGAAGAGGT TCCTTCCTGC CAAGTGGTAA AATGTTCAAG CCTGGCAGTT CCGGGAAAGA | 1620 |
| TCAACATCAG CTGCAGTGGG GAGCCCGTGT TTGGCACTGT GTGCAAGTTC GCCTGTCCTG | 1680 |
| AAGGATGGAC GCTCAATGGC TCTGCAGCTC GGACATGTGG AGCCACAGGA CACTGGTCTG | 1740 |
| GCCTGCTACC TACCTGTGAA GCTCCCACTG AGTCCAACAT TCCCTTGGTA GCTGGACTTT | 1800 |
| CTGCTGCTGG ACTCTCCCTC CTGACATTAG CACCATTTCT CCTCTGGCTT CGGAAATGCT | 1860 |
| TACGGAAAGC AAAGAAATTT GTTCCTGCCA GCAGCTGCCA AAGCCTTGAA TCAGACGGAA | 1920 |
| GCTACCAAAA GCCTTCTTAC ATCCTTTAAG TTCAAAAGAA TCAGAAACAG GTGCATCTGG | 1980 |
| GGAACTAGAG GGATACACTG AAGTTAACAG AGACAGATAA CTCTCCTCGG GTCTCTGGCC | 2040 |
| CTTCTTGCCT ACTATGCCAG ATGCCTTTAT GGCTGAAACC GCAACACCCA TCACCACTTC | 2100 |
| AATAGATCAA AGTCCAGCAG GCAAGGACGG CCTTCAACTG AAAAGACTCA GTGTTCCCTT | 2160 |
| TCCTACTCTC AGGATCAAGA AAGTGTTGGC TAATGAAGGG AAAGGATATT TTCTTCCAAG | 2220 |
| CAAAGGTGAA GAGACCAAGA CTCTGAAATC TCAGAATTCC TTTTCTAACT CTCCCTTGCT | 2280 |
| CGCTGTAAAA TCTTGGCACA GAAACACAAT ATTTTGTGGC TTTCTTTCTT TTGCCCTTCA | 2340 |
| CAGTGTTTCG ACAGCTGATT ACACAGTTGC TGTCATAAGA ATGAATAATA ATTATCCAGA | 2400 |
| GTTTAGAGGA AAAAAATGAC TAAAAATATT ATAACTTAAA AAAATGACAG ATGTTGAATG | 2460 |
| CCCACAGGCA AATGCATGGA GGGTTGTTAA TGGTGCAAAT CCTACTGAAT GCTCTGTGCG | 2520 |
| AGGGTTACTA TGCACAATTT AATCACTTTC ATCCCTATGG GATTCAGTGC TTCTTAAAGA | 2580 |
| GTTCTTAAGG ATTGTGATAT TTTTACTTGC ATTGAATATA TTATAATCTT CCATACTTCT | 2640 |
| TCATTCAATA CAAGTGTGGT AGGGACTTAA AAAACTTGTA AATGCTGTCA ACTATGATAT | 2700 |
| GGTAAAAGTT ACTTATTCTA GATTACCCCC TCATTGTTTA TTAACAAATT ATGTTACATC | 2760 |
| TGTTTTAAAT TTATTTCAAA AAGGGAAACT ATTGTCCCCT AGCAAGGCAT GATGTTAACC | 2820 |
| AGAATAAAGT TCTGAGTGTT TTTACTACAG TTGTTTTTTG AAAACATGGT AGAATTGGAG | 2880 |
| AGTAAAAACT GAATGGAAGG TTTGTATATT GTCAGATATT TTTTCAGAAA TATGTGGTTT | 2940 |
| CCACGATGAA AAACTTCCAT GAGGCCAAAC GTTTTGAACT AATAAAAGCA TAAATGCAAA | 3000 |
| CACACAAAGG TATAATTTTA TGAATGTCTT TGTTGGAAAA GAATACAGAA AGATGGATGT | 3060 |
| GCTTTGCATT CCTACAAAGA TGTTTGTCAG ATGTGATATG TAAACATAAT TCTTGTATAT | 3120 |
| TATGGAAGAT TTTAAATTCA CAATAGAAAC TCACCATGTA AAAGAGTCAT CTGGTAGATT | 3180 |
| TTTAACGAAT GAAGATGTCT AATAGTTATT CCCTATTTGT TTTCTTCTGT ATGTTAGGGT | 3240 |
| GCTCTGGAAG AGAGGAATGC CTGTGTGAGC AAGCATTTAT GTTTATTTAT AAGCAGATTT | 3300 |
| AACAATTCCA AAGGAATCTC CAGTTTTCAG TTGATCACTG GCAATGAAAA ATTCTCAGTC | 3360 |
| AGTAATTGCC AAAGCTGCTC TAGCCTTGAG GAGTGTGAGA ATCAAAACTC TCCTACACTT | 3420 |
| CCATTAACTT AGCATGTGTT GAAAAAAAAA GTTTCAGAGA AGTTCTGGCT GAACACTGGC | 3480 |
| AACGACAAAG CCAACAGTCA AAACAGAGAT GTGATAAGGA TCAGAACAGC AGAGGTTCTT | 3540 |
| TTAAAGGGGC AGAAAAACTC TGGGAAATAA GAGAGAACAA CTACTGTGAT CAGGCTATGT | 3600 |
| ATGGAATACA GTGTTATTTT CTTTGAAATT GTTTAAGTGT TGTAAATATT TATGTAAACT | 3660 |
| GCATTAGAAA TTAGCTGTGT GAAATACCAG TGTGGTTTGT GTTTGAGTTT TATTGAGAAT | 3720 |
| TTTAAATTAT AACTTAAAAT ATTTTATAAT TTTTAAAGTA TATATTTATT TAAGCTTATG | 3780 |
| TCAGACCTAT TTGACATAAC ACTATAAAGG TTGACAATAA ATGTGCTTAT GTTT | |

Seq ID NO: 75 Protein sequence:
Protein Accession #: NP_000441

| | |
|---|---|
| 1 11 21 31 41 51 | |
| \| \| \| \| \| \| | |
| MIASQELSAL TLVLLIKESG AWSYNTSTEA MTYDEASAYC QQRYTNLVAI QNKEEIEYLN | 60 |
| SILSYSPSYY WIGIRKVNNV WVWVGTQKPL TEEAKNWAPG EPNNRQKDED CVEIYIKREK | 120 |
| DVGMWNDERC SKKKLALCYT AACTNTSCSG HGECVETINN YTCKCDPGFS GLKCEQIVNC | 180 |
| TALESPEHGS LVCSHPLCNF SYNSSCSISC DRGYLPSSME TMQCMSSGEW SAPIPACNVV | 240 |
| ECDAVTNPAN GFVECFQNPG SFPWNTTCTF DCEEGFELMG AQSLQCTSSG NWDNEKPTCK | 300 |
| AVTCRAVRQP QNGSVRCSHS PAGEFTFKSS CNFTCEEGFM LQGPAQVECT TQGQWTQQIP | 360 |
| VCEAFQCTAL SNPERGYMNC LPSASGSFRY GSSCEFSCEQ GFVLKGSKRL QCGPTGEWDN | 420 |
| EKPTCEAVRC DAVHQPPKGL VRCAHSPIGE FTYKSSCAFS CEESFELYGS TQLECTSQGQ | 480 |
| WTEEVPSCQV VKCSSLAVPG KINMSCSGEP VFGTVCKFAC PEGWYLNGSA ARTCGATGHW | 540 |
| SGLLPTCEAP TESNIPLVAG LSAAGLSLLT LAPFLLWLRK CLRKAKKFVP ASSCQSLESD | 600 |
| GSYQKPSYIL | |

Seq ID NO: 76 Nucleotide sequence:
Nucleic Acid Accession #: NM_031439
Coding sequence: 69 . . . 1235 (underlined sequences correspond to
start and stop codons)

| | |
|---|---|
| 1 11 21 31 41 51 | |
| \| \| \| \| \| \| | |
| CCCGACCCGT GCGAGGGCCA GGTCCGCGCC TGCCCCGCCA GGCGAAGCGA GGCGACCCGC | 60 |
| GTGCGGCCAT GGCTTCGCTG CTGGGAGCCT ACCCTTGGCC CGAGGGTCTC GAGTGCCCGG | 120 |
| CCCTGGACGC CGAGCTGTCG GATGGACAAT CGCCGCCGGC CGTCCCCCGG CCCCCGGGGG | 180 |
| ACAAGGGCTC CGAGAGCCGT ATCCGGCGGC CCATGAACGC CTTCATGGTT TGGGCCAAGG | 240 |
| ACGAGAGGAA ACGGCTGGCA GTGCAGAACC CGGACCTGCA CAACGCCGAG CTCAGCAAGA | 300 |
| TGCTGGGAAA GTCGTGGAAG GCGCTGACGC TGTCCCAGAA GAGCCCGTAC GTGGACGAGG | 360 |
| CGGAGCGGCT GCGCCTGCAG CACATGCAGG ACTACCCCAA CTACAAGTAC CGGCCGCGCA | 420 |
| GGAAGAAGCA GGCCAAGCGG CTGTGCAAGC GCGTGGACCC GGGCTTCCTT CTGAGCTCCC | 480 |
| TCTCCCGGGA CCAGAACGCC CTGCCGGAGA AGAGAAGCGG CAGCCGGGGG GCGCTGGGGG | 540 |
| AGAAGGAGGA CAGGGGTGAG TACTCCCCCG GCACTGCCCT GCCCAGCCTC CGGGGCTGCT | 600 |
| ACCACGAGGG GCCGGCTGGT GGTGGCGGCG GCGGCACCCC GAGCAGTGTG GACACGTACC | 660 |
| CGTACGGGCT GCCCACACCT CCTGAAATGT CTCCCCTGGA CGTGCTGGAG CCGGAGCAGA | 720 |
| CCTTCTTCTC CTCCCCCTGC CAGGAGGAGC ATGGCCATCC CCGCCGCATC CCCCACCTGC | 780 |
| CAGGGCACCC GTACTCACCG GAGTACGCCC CAAGCCCTCT CCACTGTAGC CACCCCCTGG | 840 |
| GCTCCCTGGC CCTTGGCCAG TCCCCCGGCG TCTCCATGAT GTCCCCTGTA CCCGGCTGTC | 900 |
| CCCCATCTCC TGCCTATTAC TCCCCGGCCA CCTACCACCC ACTCCACTCC AACCTCCAAG | 960 |
| CCCACCTGGG CCAGCTTTCC CCGCCTCCTG AGCACCCTGG CTTCGACGCC CTGGATCAAC | 1020 |
| TGAGCCAGGT GGAACTCCTG GGGGACATGG ATCGCAATGA ATTCGACCAG TATTTGAACA | 1080 |
| CTCCTGGCCA CCCAGACTCC GCCACAGGGG CCATGGCCCT CAGTGGGCAT GTTCCGGTCT | 1140 |
| CCCAGGTGAC ACCAACGGGT CCCACAGAGA CCAGCCTCAT CTCCGTCCTG GCTGATGCCA | 1200 |
| CGGCCACGTA CTACAACAGC TACAGTGTGT CATAGAGCTG GAGGCGCCCC GTCCGGTCAG | 1260 |
| CCCTCGCGCC CTCTCCTTCT TGTGCCTTGA GTGGCAGAGG AGCCGTCCAG CCACACCAGC | 1320 |
| TTTCCTCCCA CCGCTCAGGG CAGGGAGGTC TGAACTGCGG CCCCAGAGCC TTTGGCCTAA | 1380 |
| GCTGGACTCT CCTTATCCGA CTGCCGCCTC TATCCCCTTC CCCACGTTCC AGCCCCTGCA | 1440 |
| GCCCACATTT TAAGTATATT CCTTCAAGTG AGTTTTCCTC CAGCCCCTGA GAGTTGCTGT | 1500 |
| CTCCCAGTGG AATGTTCACT GACGTCTTTT CTTGGTAGCC ATCATCGAAA CTAATGGGGG | 1560 |
| GACAGACTTG ATAGCCAAGG TCCCTTCTGG TCCAGTTTTC TGATTTAGGG TTCTCTCAAG | 1620 |
| ATTAATAAAG GAAGATGGGG AAATTTGACT CATTAATGAG CTCGCTAACC TACGATCTGG | 1680 |
| TGATAATTTT GTGTGCACAG CCCAAGGACC ACGAGGCTTT CTGCACTTTC TGCACCCCCT | 1740 |
| TCCAAAGTGA CCACAAAATT TCAAAGGGAC TCATACAATT TGAGAAAAAA CAGTCAACCT | 1800 |
| GATTTGAGAA ATTAACCAGT ATGGCTAACT ATATCACAGA AAATGGGATT GAGTTAAAAC | 1860 |
| TATTTTATTT TAAATATACA TTTTAAAGCA GTTCTTTTTT TTTGTTAATT TGTTTATTAT | 1920 |
| ACACACACTT CAAGAGCCAC CGCGCCCAGC CTACATTTAT AATTTTCATT CTCTTTTACC | 1980 |
| TATAAAATTC AGTGTATTAG TTTCATTACA TAGGAGAAAT TATATTTCTA AACATTTTAT | 2040 |
| GATGTTTAAA AACAAAACAG GCTGTTGTAA AAAAAAAAAA AAAAAAAAA | |

Seq ID NO: 77 Protein sequence:
Protein Accession #: NP_113627

| | |
|---|---|
| 1 11 21 31 41 51 | |
| \| \| \| \| \| \| | |
| MASLLCAYPW PEGLECPALD AELSDGQSPP AVPRPPGDKG SESRIRRPMN AFMVWAKDER | 60 |
| KRLAVQNPDL HNAELSKMLG KSWKALTLSQ KRPYVDEAER LRLQHMQDYP NYKYRPRRKK | 120 |
| QAKRLCKRVD PGFLLSSLSR DQNALPEKRS GSRGALGEKE DRGEYSPGTA LPSLRGCYHE | 180 |
| GPAGGGGGGT PSSVDTYPYG LPTPPEMSPL DVLEPEQTFF SSPCQEEHCH PRRIPHLPCH | 240 |
| PYSPEYAPSP LHCSHPLGSL ALGQSPGVSM MSPVPGCPPS PAYYSPATYH PLHSNLQAHL | 300 |
| GQLSPPPEHP GFDALDQLSQ VELLGDMDRN EFDQYLNTPG HPDSATGANA LSGHVPVSQV | 360 |
| TPTGPTETSL ISVLADATAT YYNSYSVS | |

Seq ID NO: 78 Nucleotide sequence:
Nucleic Acid Accession #: XM_035787
Coding sequence: 329 . . . 949 (underlined sequences correspond to
start and stop codons)

| | |
|---|---|
| 1 11 21 31 41 51 | |
| \| \| \| \| \| \| | |
| TGCCCCGCCC CGCTCCCCAG CGCCCCGGAA GTGATCTGTG GCGGCTGCTG CAGAGCCGCC | 60 |
| AGGAGGAGGG TGGATCTCCC CAGAGCAAAG CGTCGGAGTC CTCCTCCTCC TTCTCCTCCT | 120 |
| CCTCCTCCTC CTCCTCCAGC CGCCCAGGCT CCCCCGCCAC CCGTCAGACT CCTCCTTCGA | 180 |
| CCGCTCCCGG CGCGGGGCCT TCCAGGCGAC AAGGACCGAG TACCCTCCGG CCGGAGCCAC | 240 |
| CCAGCCGCGG CTTCCGGAGC CCTCGGGGCG GCGGACTGGC TCGCGGTGCA GATTCTTCTT | 300 |
| AATCCTTTGG TGAAAACTGA GACACAAAAT GGCTGCAAAT AAGCCCAAGG GTCAGAATTC | 360 |
| TTTGGCTTTA CACAAAGTCA TCATGGTGGG CAGTGGTGGC GTGGGCAAGT CAGCTCTGAC | 420 |
| TCTACAGTTC ATGTACGATG AGTTTGTGGA GGACTATGAG CCTACCAAAG CAGACAGCTA | 480 |
| TCGGAAGAAG GTAGTGCTAG ATGGGGAGGA AGTCCAGATC GATATCTTAG ATACAGCTGG | 540 |
| GCAGGAGGAC TACGCTGCAA TTAGAGACAA CTACTTCCGA AGTGGGGAGG GGTTCCTCTG | 600 |
| TGTTTTCTCT ATTACAGAAA TGGAATCCTT TGCAGCTACA GCTGACTTCA GGGAGCAGAT | 660 |
| TTTAAGAGTA AAAGAAGATG AGAATGTTCC ATTTCTACTG GTTGGTAACA AATCAGATTT | 720 |
| AGAAGATAAA AGACAGGTTT CTGTAGAAGA GGCAAAAAAC AGAGCTGAGC AGTGGAATGT | 780 |
| TAACTACGTG GAAACATCTG CTAAAACACG AGCTAATGTT GACAAGGTAT TTTTTGATTT | 840 |
| AATGACAGAA ATTCGAGCGA GAAAGATGGA AGACAGCAAA GAAAAGAATG GAAAAAAGAA | 900 |
| GAGGAAAAGT TTAGCCAAGA GAATCAGAGA AAGATGCTGC ATTTTATAAT CAAAGCCCAA | 960 |
| ACTCCTTTCT TATCTTGACC ATACTAATAA ATATAATTTA TAAGCATTGC CATTGAAGGC | 1020 |
| TTAATTGACT GAAATTACTT TAACATTTTG GAAATTGTTG TATATCACTA AAAGCATGAA | 1080 |
| TTGGAACTGC AATGAAAGTC AAATTTACTT TAAAAAGAAA TTAATATGGC TTCACCAAGA | 1140 |
| AGCAAAGTTC AACTTATTTC ATAATTGCCT ACATTTATCA TGGTCCTGAA TGTAGCGTGT | 1200 |
| AAGCTTGTGT TTCTTGGGCA GTCTTTCTTG AAATTGAAGA GGTGAAATGG GGGTGGGGAG | 1260 |
| TGGGAGGAAA GGTGACTTCC TCTGGTGTTT ATTATAAAGC TTAAATTTTA TATCATTTTA | 1320 |
| AAATGTCTTG GTCTTCTACT GCCTTGAAAA ATGACAATTG TGAACATGAT AGTTAAACTA | 1380 |
| CCACTTTTTT TAACCATTAT TATGCAAAAT TTAGAAGAAA AGTTATTGGC ATGGTTGTTG | 1440 |
| CATATAGTTA AACTGAGAGT AATTCATCTG TGAATCTGCT TTAATTACCT GGTGAGTAAC | 1500 |
| TTAGAAAAGT GGTGTAAACT TGTACATGGA ATTTTTTGAA TATGCCTTAA TTTAGAAACT | 1560 |
| GAAAAATATC TGGTTATATC ATTCTGGGTG TGTTCTTACT GACACCAGGG GTCCGCTGCC | 1620 |
| CCATGTGTCC TGGTGAGAAA ATATATGCCT GGCACAGCTT TTGTATAGAA AATTCTTGAG | 1680 |
| AAGTAACTGT CCGCTAGAAG TCTGTCCAAA TTTAAAATGT GTGCCATATT CTGGTTCTTG | 1740 |
| AAAATAAGAT TCCAGAGCTC TTTGATCGCT TTTAATAAAC TGCAAGTTCA TTTTAAATGA | 1800 |
| AGGGCCAGCA TATATACTTG CAAGATAATT TTCAGCTGCA AGGATTCAGC ACCAGTTATG | 1860 |
| TTTGAATGAA CCCTCCTTTT CTCTGAGATT CTGGTCCCTG GAAATCCCTT TCTGCTAGTG | 1920 |
| GTGAGCATGT AAGTGTTAAG TTTTTAATCT GGGAGCAGGG CATAGGAAGA AAATGTCAGT | 1980 |
| AGTGCTAATG CATTTTGCAC TAGAACGCTT CGGGAAAATA TTCATGCTTG CCATCTGTTC | 2040 |
| ATTTGTAAAT TTATATTCAT AAAGTTACAG TTTGATACAG GAATTATTAG GAGTAATTCT | 2100 |
| TTTCTGTTTC TGTTTATAAT GAAGAACACT GTAGCTACAT TTTCAGAAGT TAACATCAAG | 2160 |
| CCATCAAACC TGGGTATAGT GCAGAAAACG TGGCACACAC TGACCACACA TTAGGCTGTG | 2220 |
| TCACCATTGT GTGGTGTACC TGCTGGAAGA ATTCTAGCAT GCTACTTGGG GACATAATTT | 2280 |
| CAGTGGGAAA TATGCCACTG ACCGATTTTT TTTTTTTCCT CTTTGCAGTG GGGCTAGGAC | 2340 |
| AGTTGATTCA ACAAAGTATT TTTTTCTTTT TTCTCAGTCC TAATTTGAAC AGGTCAAAGA | 2400 |
| TGTGTTCAGG CATTCCAGGT AACAGGTGTG TATGTAAAGT TAAAAATAGG CTTTTTAGGA | 2460 |
| ACTCACTCTT TAGATATTTA CATCCAGCTT CTCATGTTAA ATATTTGTCC TTAAAGGGTT | 2520 |
| TGAGATGTAC ATCTTTCATT TCGTATTTCT CATAGGCTAT GCCATGTGCG GAATTCAAGT | 2580 |
| TACCAATGTA ACACTGGCCA GCGGGCCCAG CAATCTCCAT GTGTACTTAT TACAGTCTTA | 2640 |
| TTTAACCAGG GGTCCTAACC ACTAACATTG TGACTTTGCT TTGAGACCTT TCCTCTCCTG | 2700 |
| GGTACTGAGG TGCTATGAAG CCAACTGACA AAGATGCATC ACGTGTCTTA GGCTGATGCC | 2760 |
| ACTACCCGAT TTGTTTATTT GCAATTTGAG CCATTTAAAG ACCAATAAAC TTCCTTTTTT | |

Seq ID NO: 79 Protein sequence:
Protein Accession #: XP_035787

| | |
|---|---|
| 1 11 21 31 41 51 | |
| \| \| \| \| \| \| | |
| MAANKPKGQN SLALHKVIMV GSGGVGKSAL TLQFMYDEFV EDYEPTKADS YRKKVVLDGE | 60 |
| EVQIDILDTA GQEDYAAIRD NYFRSGEGFL CVFSITEMES FAATADFREQ ILRVKEDENV | 120 |
| PFLLVGNKSD LEDKRQVSVE EAKNRASQWN VNYVETSAKT RANVDKVFFD LMREIRARKM | 180 |
| EDSKEKNGKK KRKSLAKRIR ERCCIL | |

Seq ID NO: 80 Nucleotide sequence:
Nucleic Acid Accession #: NM_003467
Coding sequence: 89 . . . 1147 (underlined sequences correspond to
start and stop codons)

| | |
|---|---|
| 1 11 21 31 41 51 | |
| \| \| \| \| \| \| | |
| GTTTGTTGGC TGCGGCAGCA GGTAGCAAAG TGACGCCGAG GGCCTGAGTG CTCCAGTAGC | 60 |
| CACCGCATCT GGAGAACCAG CGGTTACCAT GGAGGGGATC AGTATATACACTTCAGATAA | 120 |
| CTACACCGAG GAAATGGGCT CAGGGGACTA TGACTCCATG AAGGAACCCT GTTTCCGTGA | 180 |
| AGAAAATGCT AATTTCAATA AAATCTTCCT GCCCACCATC TACTCCATCA TCTTCTTAAC | 240 |
| TGGCATTGTG GGCAATGGAT TGGTCATCCT GGTCATGGGT TACCAGAAGA AACTGAGAAG | 300 |
| CATGACGGAC AAGTACAGGC TGCACCTGTC AGTGGCCGAC CTCCTCTTTG TCATCACGCT | 360 |
| TCCCTTCTGG GCAGTTGATG CCGTGGCAAA CTGGTACTTT GGGAACTTCC TATGCAAGGC | 420 |
| AGTCCATGTC ATCTACACAG TCAACCTCTA CAGCAGTGTC CTCATCCTGG CCTTCATCAG | 480 |
| TCTGGACCGC TACCTGGCCA TCGTCCACGC CACCAACAGT CAGAGGCCAA GGAAGCTGTT | 540 |
| GGCTGAAAAG GTGGTCTATG TTGGCGTCTG GATCCCTGCC CTCCTGCTGA CTATTCCCGA | 600 |
| CTTCATCTTT GCCAACGTCA GTGAGGCAGA TGACAGATAT ATCTGTGACC GCTTCTACCC | 660 |
| CAATGACTTG TGGGTGGTTG TGTTCCAGTT TCAGCACATC ATGGTTGGCC TTATCCTGCC | 720 |
| TGGTATTGTC ATCCTGTCCT GCTATTGCAT TATCATCTCC AAGCTGTCAC ACTCCAAGGG | 780 |
| CCACCAGAAG CGCAAGGCCC TCAAGACCAC AGTCATCCTC ATCCTGGCTT TCTTCGCCTG | 840 |
| TTGGCTGCCT TACTACATTG GGATCAGCAT CGACTCCTTC ATCCTCCTGG AAATCATCAA | 900 |
| GCAAGGGTGT GAGTTTGAGA ACACTGTGCA CAAGTGGATT TCCATCACCG AGGCCCTAGC | 960 |
| TTTCTTCCAC TGTTGTCTGA ACCCCATCCT CTATGCTTTC CTTGGAGCCA AATTTAAAAC | 1020 |
| CTCTGCCCAG CACGCACTCA CCTCTGTGAG CAGAGGGTCC AGCCTCAAGA TCCTCTCCAA | 1080 |
| AGGAAAGCGA GGTGGACATT CATCTGTTTC CACTGAGTCT GAGTCTTCAA GTTTTCACTC | 1140 |
| CAGCTAACAC AGATGTAAAA GACTTTTTTT TATACGATAA ATAACTTTTT TTTAAGTTAC | 1200 |
| ACATTTTTCA GATATAAAAG ACTGACCAAT ATTGTACAGT TTTTATTGCT TGTTGGATTT | 1260 |
| TTGTCTTGTG TTTCTTTAGT TTTTGTGAAG TTTAATTGAC TTATTTATAT AAATTTTTTT | 1320 |
| TGTTTCATAT TGATGTGTGT CTAGGCAGGA CCTGTGGCCA AGTTCTTAGT TGCTGTATGT | 1380 |
| CTCGTGGTAG GACTGTAGAA AAGGGAACTG AACATTCCAG AGCGTGTAGT GAATCACGTA | 1440 |
| AAGCTAGAAA TGATCCCCAG CTGTTTATGC ATAGATAATC TCTCCATTCC CGTGGAACGT | 1500 |
| TTTTCCTGTT CTTAAGACGT GATTTTGCTG TAGAAGATGG CACTTATAAC CAAAGCCCAA | 1560 |
| AGTGGTATAG AAATGCTGGT TTTTCAGTTT TCAGGAGTGG GTTGATTTCA GCACCTACAG | 1620 |
| TGTACAGTCT TGTATTAAGT TGTTAATAAA AGTACATGTT AAACTTACTT AGTGTTATG | |

Seq ID NO: 81 Protein sequence:
Protein Accession #: NP_003458

| | |
|---|---|
| 1 11 21 31 41 51 | |
| \| \| \| \| \| \| | |
| NEGISIYTSD NYTEEMGSGD YDSMKEPCFR EENANFNKIF LPTIYSTIFL TGIVGNGLVI | 60 |
| LVMGYQKKLR SMTDKYRLHL SVADLLFVIT LPFWAVDAVA NWYFGNFLCK AVHVIYTVNL | 120 |
| YSSVLILAFI SLDRYLAIVH ATNSQRPRKL LAEKVVYVGV WIPALLLTIP GFIFANVSEA | 180 |
| DDRYICDRFY PNDLWVVVFQ FQHIMVGLIL PGIVILSCYC IIISKLSHSK GHQKRKALKT | 240 |
| TVILILAFFA CWLPYYIGIS IGSFILLETI KQGCEFENTV HKWISITEAL AFFNCCLNPI | 300 |
| LYAFLGAKFK TSAQHALTSV SRGSSLKILS KGKRCGHSSV STESESSSFH SS | |

Seq ID NO: 82 Nucleotide sequence:
Nucleic Acid Accession #: NM_014959
Coding sequence: 314 . . . 1609 (underlined sequences correspond to
start and stop codons)

| | |
|---|---|
| 1 11 21 31 41 51 | |
| \| \| \| \| \| \| | |
| CTGGTTCTCA ACTTCTTTTG AAATAATGTT CATAGAGAAG GAGGGCTGTC TGAGATTCGA | 60 |
| GGGAAACAAG CTCTCAGGAC TTCCGGTCGC CATGATGGCT GTGGGCGGTA AACGCGGTTA | 120 |
| GTGCAAGCAT CTGGGCCATC TTCAATGGTA AAAAAGATAC AGTAAAGACA TAAATACCAC | 180 |
| ATTTGACAAA TGGAAAAAAA GGAGTGTCCA GAAAAGAGTA GCAGCAGTGA GGAAGAGCTC | 240 |
| CCGAGACGGG TATACAGGGA GCTACCCTGT GTTTCTGAGA CCCTTTGTGA CATCTCACAT | 300 |
| TTTTTCCAAG AAGATGATGA GACAGAGGCA GAGCCATTAT TGTTCCGTGC TGTTCCTGAG | 360 |
| TGTCAACTAT CTGGGGGGGA CATTCCCAGG AGACATTTGC TCAGAAGAGA ATCAAATAGT | 420 |
| TTCCTCTTAT GCTTCTAAAG TCTGTTTTGA GATCGAAGAA GATTATAAAA ATCGTCAGTT | 480 |
| TCTGGGGCCT GAAGGAAATG TGGATGTTGA GTTGATTGAT AAGAGCACAA ACAGATACAG | 540 |
| CGTTTGGTTC CCCACTGCTG GCTGGTATCT GTGGTCAGCC ACAGGCCTCG GCTTCCTGGT | 600 |
| AAGGGATGAG GTCACAGTGA CGATTGCGTT TGGTTCCTGG AQTCAGCACC TGGCCCTGGA | 660 |
| CCTGCAGCAC CATGAACAGT GGCTGGTGGG CGGCCCCTTG TTTGATGTCA CTGCAGAGCC | 720 |
| AGAGGAGGCT GTCGCCGAAA TCCACCTCCC CCACTTCATC TCCCTCCAAG GTGAGGTGGA | 780 |
| CGTCTCCTGG TTTCTCGTTG CCCATTTTAA GAATGAAGGG ATGGTCCTGG AGCATCCAGC | 840 |
| CCGGGTGGAG CCTTTCTATG CTGTCCTGGA AAGCCCCAGC TTCTCTCTGA TGGGCATCCT | 900 |
| GCTGCGGATC GCCAGTGGGA CTCGCCTCTC CATCCCCATC ACTTCCAACA CATTGATCTA | 960 |
| TTATCACCCC CACCCCGAAG ATATTAAGTT CCACTTGTAC CTTGTCCCCA GCGACGCCTT | 1020 |
| GCTAACAAAG GCGATAGATG ATGAGGAAGA TCGCTTCCAT GGTGTGCGCC TGCAGACTTC | 1080 |
| GCCCCCAATG GAACCCCTGA ACTTTGGTTC CAGTTATATT GTGTCTAATT CTGCTAACCT | 1140 |
| GAAAGTAATG CCCAAGGAGT TGAAATTGTC CTACAGGAGC CCTGGAGAAA TTCAGCACTT | 1200 |
| CTCAAAATTC TATGCTGGGC AGATGAAGGA ACCCATTCAA CTTGAGATTA CTGAAAAAAG | 1260 |
| ACATGGGACT TTGGTGTGGG ATACTGAGGT GAAGCCAGTG GATCTCCAGC TTGTAGCTGC | 1320 |
| ATCAGCCCCT CCTCCTTTCT CAGGTGCAGC CTTTGTGAAG GAGAACCACC GGCAACTCCA | 1380 |
| AGCCAGGATG GGGGACCTGA AAGGGGTGCT CGATGATCTC CAGGACAATG AGGTTCTTAC | 1440 |
| TGAGAATGAG AAGGAGCTGG TGGAGCAGGA AAAGACACGG CAGAGCAAGA ATGAGGCCTT | 1500 |
| GCTGAGCATG GTGGAGAAGA AAGGGGACCT GGCCCTGGAC GTGCTCTTCA GAAGCATTAG | 1560 |
| TGAAAGGGAC CCTTACCTCG TGTCCTATCT TAGACAGGAG AATTTGTAAA ATGAGTCAGT | 1620 |
| TAGGTAGTCT GGAAGAGAGA ATCCAGCGTT CTCATTGGAA ATGGATAAAC AGAAATGTGA | 1680 |
| TCATTGATTT CAGTGTTCAA GACAGAAGAA GACTGGGTAA CATCTATCAC ACAGGCTTTC | 1740 |
| AGGACAGACT TGTAACCTGG CATGTACCTA TTGACTGTAT CCTCATGCAT TTTCCTCAAG | 1800 |
| AATGTCTGAA GAAGGTAGTA ATATTCCTTT TAAATTTTTT CCAACCATTG CTTGATATAT | 1860 |
| CACTATTTTA TCCATTGACA TGATTCTTGA AGACCCAGGA TAAAGGACAT CCGGATAGGT | 1920 |
| GTGTTTATGA AGGATGGGGC CTGGAAAGGC AACTTTTCCT GATTAATGTG AAAAATAATT | 1980 |
| CCTATGGACA CTCCGTTTGA AGTATCACCT TCTCATAACT AAAAGCAGAA AAGCTAACAA | 2040 |
| AAGCTTCTCA GCTGAGGACA CTCAAGGCAT ACATGATGAC AGTCTTTTTT TTTTTTGTAT | 2100 |
| GTTAGGACTT TAACACTTTA TCTATGGCTA CTGTTATTAG AACAATGTAA ATGTATTTGC | 2160 |
| TGAAAGAGAG CACAAAAATG GGAGAAAATG CAAACATGAG CAGAAAATAT TTTCCCACTG | 2220 |
| GTGTGTAGCC TGCTACAAGG AGTTGTTGGG TTAAATGTTC ATGGTCAACT CCAAGGAATA | 2280 |
| CTGAGATGAA ATGTGGTAAA TCAACTCCAC AGAACCACCA AAAAGAAAAT GAGGGTAATT | 2340 |
| CAGCTTATTC TGAGACAGAC ATTCCTGGCA ATGTACCATA CAAAAAATAA GCCAACTCTG | 2400 |
| ACATTTGGAT TCTACCATAG ACTCTGTCAT TTTGTAGCCA TTTCAGCTGT CTTTTGATTA | 2460 |
| ATGTTTTCGT GGCACACATA TTTCCATCCT TTTATGTTTA ATCTGTTTAA AACAAGTTCC | 2520 |
| TAGTAGACAC CATCTGGTTG AGTCAGTTTT TTTTATGGTG TATTTTGAAC CCATTCTGAT | 2580 |
| AGTCTCTTTT AACTGGAAGA TTTCAATTAC TTACGTTAAT GTAATTATTA ATATGTTAGG | 2640 |
| ATTTATCCTC AGTCAGCCAG TTTGTTATGT CTTTTCTATT CTACTGTTAT CACATTTGTA | 2700 |
| CCACTTAAAG TGGAATCTAG GCACTTTATC ACCATTTAGA TCCTATTACC TTTTCTCATC | 2760 |
| TAGGATATAG TTATCTTCTA CATAATCTTT CTGTATCTTA AAACCCATCA ATAAATTATT | 2820 |
| ATATATTTTC TACTTTTAAT CACTCAGAAG ATTTAAAAAA CTCATGAGAA GAGTAATCTG | 2880 |
| TTATGTTTTT CCAGATATTT ACCATTTCTG TTGCTCTTCC TTCATTATTT TCCAAATTTC | 2940 |
| GTTCTGCAAA TTTCCACTTC TTCTGATAGA CGTTTTTTAG TTCTTTTAGA GTGGTTCTGA | 3000 |
| TAGGTACAGA TTCTCTTATT TTTTGCTTCC TCTGAGGACA TCTTTTTCTC ACCTTCATTC | 3060 |
| TCAGTGATGT TTTTTGCTTG TAGTATTTTT AGTTGACATT GTTTTCTGTT CAGCAGTTTC | 3120 |
| CTTTTAGCTT CCGTATTTCC TGATGAGAAA TCTGCAGTCA TTCAAATTGT TGTTTCCCTG | 3180 |
| TATGTAGTGT GTCATTTTTC TGTCAGATTT CAAGGTATTT ATCTTTAGTT TTTAGCCATT | 3240 |
| TCATTATGTT GGGGATGAGT TTCCTTGTTT TATTCCCTTT GGAATTTGCT CCAATTCATA | 3300 |
| AATTTGCAGT TTTATGTCTT TTACCAAACT TAGAGGTTTT CAGCCTAATT TCTAAAAATA | 3360 |
| CTTTTTATTA GCCTGATTTT CATCTTTATA GGAAATAGTT TAAGTGATGA CAAGTTCCAA | 3420 |
| TAGCTTATAT GCCCAGAAGG CCTTCAAAAT AAGAATTTTG AAAGAATACA GAAAACAAAC | 3480 |
| TTTTATATCC TTCTCATGTC TTCTACTGTA AAATTCATAT GCTTTGCTAC TCTAAACCTA | 3540 |
| GTTTGAAATC AACAGTCTTG AGAATAGATG AAAATTTTGA TGAATAGTGG AATTCTTTTA | 3600 |
| AATGGAAACC TCTTACATGT GATTTTCCTT GCCATCTAGA AATAAACCAT AGTATTTATG | 3660 |
| TTGAATCAAT CAATATTATA TTTTGTTTTT TTCCTCCTCT TCTGAGACTC TTATTGTGGA | 3720 |
| AATGTTAGAC TTTTATGTTT TCCTAAATGT CCCTGATATT CTACTTATTT AGAACATCTT | 3780 |
| TTCATTTTTT CCATTATTCT GATTGGGTAA TTTTAATTTG TCTATTTTCA AATTTGCTGG | 3840 |
| AGTGTTCACC TGTTGTTGTC TGTGTCGTCC CACTGAGTGC ATTCACCACC TTTTAAATTT | 3900 |
| TGGTCACTGT ATGTATCAGT TCTAAAATTT CCATTTTGTT CTCTATATTT TAAATTTCTT | 3960 |
| GGCTTATATT CTATTTTCCT GCAAATGTGT CAGCATTTGC TTGTTTGAGC TTTTTTTTTT | 4020 |
| TCAAGACAGG GTCTCAACTC TGTTACCCAG GCTGGAGTGC AGTGGTGCGA TCTCAGCTCA | 4080 |
| CTGCAACCTC TGCCTCCTGG TTCAAGCGAT TATTGTGCCT CAGCCTCCTG AGTAGCTGGG | 4140 |
| ATTACAGGCA TGCACCACCA CAGCCCAGCT AATTTTTTGT ATTTTTAGTA GAGACAGAGT | 4200 |
| TTTGCTATGT TGGCCAGGCT GGTTTTGAAC TCCTCCCCTC AAGTGATCCA CCCACCTCAG | 4260 |
| CCTCCCAAAG TGCTGGGATT ACAGGCCACT ACACCTGGCA CATTTGAGTA TTTTTTTTTT | 4320 |
| TTTTTTTTTT TTGAGATGGA GTCTCGCTCT GTCATCTACG CTGGAGTGCA GTGGTGTGAT | 4380 |
| CTCAGCTCAC TGCAGCCTCT GTCTCCCGGG CTCAAGCGAT TCTCTTGCCT CAGCCTCCTG | 4440 |
| AGTAGCTAGG ACTACAGGTG CATGCCAACA CGCCCGGCTA ATTTTTTTAA AAAATATTTT | 4500 |
| TAGTAGAGAC AGGGTTTCAC CATTTTGGCC AGGATGGTCT CGATCTCCTG ACCTCATGAT | 4560 |
| CCACCCGCCT CGGCCTTCCA AAGTGCTGGG ATTACAGGCA TGAGCCACCG TGCCTGGCCT | 4620 |
| CATTTGAGTA TTTTTATAAT GTCTCTTTTA AAGTCTTTGT CAGATAATTC CACTGTACAT | 4680 |
| GTTATTCAGT GTTTGGTGTC CACTGAGTTG TCATTTGCCA GACAAGTGGA GATTTTTGCA | 4740 |
| GCTCATCCTT GTATTCTCAG TAGTTCCGAT ATGTACCCTC GACATGTGAA TGTTATCTTA | 4800 |
| TGAGACTCTG TTTTATTTGT ATCCAACAGA AGATGTTTAT TATTTATTTG GCTTTCTGTG | 4860 |
| AACTGAGGTC TTAATATCAG CTCATTTTAA AAGTCTTTGC AGTGGTATTC GGATCTATCC | 4920 |
| TGTGTGTGCC TATGAGATTG GGTGCAGTGT ATCCTGTTAG CTCCATTCTC AGGGCGTTTG | 4980 |
| AATGTGAATT AGGACCAGCG CAATGAATGC TCAAGTTGGG GTTGGGCGTT AGAATTCATA | 5040 |
| AAAGTCTTTA TATGCTCAG | |

Seq ID NO: 83 Protein sequenc:e
Protein Accession #: NP_055774

| | |
|---|---|
| 1 11 21 31 41 51 | |
| \| \| \| \| \| \| | |
| MMRQRQSHYC SVLFLSVNYL GGTFPGDICS EENQIVSSYA SKVCFEIEED YKNRQFLGPE | 60 |
| GNVDVELIDK STNRYSVWFP TAGWYLWSAT GLGFLVRDEV TVTIAFGSWS QHLALDLQHH | 120 |
| EQWLVGGPLF DVTAEPEEAV AEIHLPHFIS LQGEVDVSWF LVAHFKNEGM VLEHPARVEP | 180 |
| FYAVLESPSF SLMGILLRIA SGTRLSIPIT SNTLIYYHPH PEDIKFHLYL VPSDALLTKA | 240 |
| IDDEEDRFEG VRLQTSPPME PLNFGSSYIV SNSANLKVMP KELKLSYRSP GEIQHFSKFY | 300 |
| ACQMKEPIQL EITEKRHGTL VWDTEVKPVD LQLVAASAPP PFSGAAFVKE NHRQLQARMG | 360 |
| DLKGVLDDLQ DNEVLTENEK ELVEQEKTRQ SKNEALLSMV EKKGDLALDV LFRSISERDP | 420 |
| YLVSYLRQQN L | |

Seq ID NO: 84 Nucleotide sequence:
Nucleic Acid Accession #: NM_007036
Coding sequence: 56-610 (underlined sequences correspond to start
and stop codons)

| | |
|---|---|
| 1 11 21 31 41 51 | |
| \| \| \| \| \| \| | |
| CTTCCCACCA GCAAAGACCA CGACTGGAGA GCCGAGCCGG AGGCAGCTGG GAAACATGAA | 60 |
| GAGCGTCTTG CTGCTGACCA CGCTCCTCGT GCCTGCACAC CTGGTGGCCG CCTGGAGCAA | 120 |
| TAATTATGCG GTGGACTGCC CTCAACACTG TGACAGCAGT GAGTGCAAAA GCAGCCCGCG | 180 |
| CTGCAAGAGG ACAGTGCTCG ACGACTGTGG CTGCTGCCGA GTGTGCGCTG CAGGGCGGGG | 240 |
| AGAAACTTGC TACCGCACAG TCTCAGGCAT GGATGGCATG AACTGTGGCC CGGGGCTGAG | 300 |
| GTGTCAGCCT TCTAATCGGG AGGATCCTTT TGGTGAAGAG TTTGGTATCT GCAAAGACTG | 360 |
| TCCCTACGGC ACCTTCGGGA TGGATTGCAG AGAGACCTGC AACTGCCAGT CAGGCATCTG | 420 |
| TGACAGGGGG ACGGGAAAAT GCCTGAAATT CCCCTTCTTC CAATATTCAG TAACCAAGTC | 480 |
| TTCCAACAGA TTTGTTTCTC TCACGGAGCA TGACATGGCA TCTGGAGATG GCAATATTGT | 540 |
| GAGAGAAGAA GTTGTGAAAG AGAATGCTGC CGGGTCTCCC GTAATGAGGA AATGGTTAAA | 600 |
| TCCACGCTGA TCCCGGCTGT GATTTCTGAG AGAAGGCTCT ATTTTCGTGA TTGTTCAACA | 660 |
| CACAGCCAAC ATTTTAGGAA CTTTCTAGAT ATAGCATAAG TACATGTAAT TTTTGAAGAT | 720 |
| CCAAATTGTG ATGCATGGTG GATCCAGAAA ACAAAAAGTA GGATACTTAC AATCCATAAC | 780 |
| ATCCATATGA CTGAACACTT GTATGTGTTT GTTAAATATT CGAATGCATG TAGATTTGTT | 840 |
| AAATGTGTGT GTATAGTAAC ACTGAAGAAC TAAAAATGCA ATTTAGGTAA TCTTACATGG | 900 |
| AGACAGGTCA ACCAAAGAGG GAGCTAGGCA AAGCTGAAGA CCGCAGTGAG TCAAATTAGT | 960 |
| TCTTTGACTT TGATGTACAT TAATGTTGGG ATATGGAATG AAGACTTAAG AGCAGGAGAA | 1020 |
| GATGGGGAGG GGGTGGGAGT GGGAAATAAA ATATTTAGCC CTTCCTTGGT AGGTAGCTTC | 1080 |
| TCTAGAATTT AATTGTGCTT TTTTTTTTTT TTTGGCTTTG GGAAAAGTCA AAATAAAACA | 1140 |
| ACCAGAAAAC CCCTGAAGGA AGTAAGATGT TTGAAGCTTA TGGAAATTTG AGTAACAAAC | 1200 |
| AGCTTTGAAC TGAGAGCAAT TTCAAAAGGC TGCTGATGTA GTTCCCGGGT TACCTGTATC | 1260 |
| TGAAGGACGG TTCTGGGGCA TAGGAAACAC ATACACTTCC ATAAATAGCT TTAACGTATG | 1320 |
| CCACCTCAGA GATAAATCTA AGAAGTATTT TACCCACTGG TGGTTTGTGT GTGTATGAAG | 1380 |
| GTAAATATTT ATATATTTTT ATAAATAAAT GTGTTAGTGC AAGTCATCTT CCCTACCCAT | 1440 |
| ATTTATCATC CTCTTGAGGA AACAAATCTA GTATTATTTG TTGAAAATGG TTAGAATAAA | 1500 |
| AACCTATGAC TCTATAAGGT TTTCAAACAT CTGAGGCATG ATAAATTTAT TATCCATAAT | 1560 |
| TATAGGAGTC ACTCTGGATT TCAAAAAATG TCAAAAAATG AGCAACAGAG GGACCTTATT | 1620 |
| TAAACATAAG TGCTGTGACT TCGGTGAATT TTCAATTTAA GGTATGAAAA TAAGTTTTTA | 1680 |
| GGAGGTTTGT AAAAGAAGAA TCAATTTTCA GCAGAAAACA TGTCAACTTT AAAATATAGG | 1740 |
| TGGAATTAGG AGTATATTTg AAAGAATCTT AGCACAAACA GGACTGTTGT ACTAGATGTT | 1800 |
| CTTAGGAAAT ATCTCAGAAG TATTTTATTT GAAGTGAAGA ACTTATTTAA GAATTATTTC | 1860 |
| AGTATTTACC TGTATTTTAT TCTTGAAGTT GGCCAACAGA GTTGTGAATG TGTGTGGAAG | 1920 |
| GCCTTTGAAT GTAAAGCTGC ATAAGCTGTT AGGTTTTGTT TTAAAAGGAC ATGTTTATTA | 1980 |
| TTGTTCAATA AAAAAGAACA AGATAC | |

Seq ID NO: 85 Protein sequence:
Protein Accession #: NP_008967.1

| | |
|---|---|
| 1 11 21 31 41 51 | |
| \| \| \| \| \| \| | |
| MKSVLLLTTL LVPAHLVAAW SNNYAVDCPQ HCDSSECKSS PRCKRTVLDD CGCCRVCAAG | 60 |
| RGETCYRTVS GMDGMKCGPG LRCQPSNGED PFGEEFGICK DCPYGTFGMD CRETCNCQSG | 120 |
| ICDRGTGKCL KFPFFQYSVT KSSNRFVSLT EHDMASGDGN IVREEVVKEN AAGSPVMRKW | 180 |
| LNPR | |

Seq ID NO: 86 Nucleotide sequence:
Nucleic Acid Accession #: D86983
Coding sequence: 52-4491 (underlined sequences correspond to start and stop codons)

| | |
|---|---|
| 1 11 21 31 41 51 | |
| \| \| \| \| \| \| | |
| AGCCGGCCGT GGTGGCTCCG TGCGTCCGAG CGTCCGTCCG CGCCGTCGGC CATGGCCAAG | 60 |
| CGCTCCAGGG GCCCCGGGCG CCGCTGCCTG TTGGCGCTCG TGCTGTTCTG CGCCTGGGGG | 120 |
| ACGCTGGCCG TGGTGGCCCA GAACCCGGGC GCAGGGTGTC CGAGCCGCTG CCTGTGCTTC | 180 |
| CGCACCACCG TGCGCTGCAT GCATCTGCTG CTGGAGGCCG TGCCCGCCGT GGCGCCGCAG | 240 |
| ACCTCCATCC TAGATCTTCG CTTTAACAGA ATCAGAGAGA TCCAACCTGG GGCATTCAGG | 300 |
| CGGCTGAGGA ACTTGAACAC ATTGCTTCTC AATAATAATC AGATCAAGAG GATACCTAGT | 360 |
| GGAGCATTTG AAGACTTGGA AAATTTAAAA TATCTCTATC TGTACAAGAA TGAGATCCAG | 420 |
| TCAATTGACA GGCAAGCATT TAAGGGACTT GCCTCTCTAG AGCAACTATA CCTGCACTTT | 480 |
| AATCAGATAG AAACTTTGGA CCCAGATTCG TTCCAGCATC TCCCGAAGCT CGAGAGGCTA | 540 |
| TTTTTGCATA ACAACCGGAT TACACATTTA GTTCCAGGGA CATTTAATCA CTTGGAATCT | 600 |
| ATGAAGAGAT TGCGACTGGA CTCAAACACA CTTCACTGCG ACTGTGAAAT CCTGTGGTTG | 660 |
| GCGGATTTGC TGAAAACCTA CGCGGAGTCG GGGAACGCGC AGGCAGCGGC CATCTGTGAA | 720 |
| TATCCCAGAC GCATCCAGGG ACGCTCAGTG GCAACCATCA CCCCGGAAGA GCTGAACTGT | 780 |
| GAAAGGCCCC GGATCACCTC CGAGCCCCAG GACGCAGATG TGACCTCGGG GAACACCGTG | 840 |
| TACTTCACCT GCAGAGCCGA AGGCAACCCC AAGCCTGAGA TCATCTGGCT GCGAAACAAT | 900 |
| AATGAGCTGA GCATCAAGAC AGATTCCCGC CTAAACTTGC TGCACGATGG GACCCTGATG | 960 |
| ATCCAGAACA CACAGGAGAC AGACCAGGGT ATCTACCAGT GCATGGCAAA GAACGTGGCC | 1020 |
| GGAGAGGTGA AGACGCAAGA GGTGACCCTC AGGTACTTCG GGTCTCCAGC TCGACCCACT | 1080 |
| TTTGTAATCC AGCCACAGAA TACAGAGGTG CTGGTTGGGG AGAGCGTCAC GCTGGAGTGC | 1140 |
| AGCGCCACAG GCCACCCCCC GCCGCGGATC TCCTGGACGA GAGGTGACCG CACACCCTTG | 1200 |
| CCAGTTGACC CGCGGGTGAA CATCACGCCT TCTGGCGGGC TTTACATACA GAACGTCGTA | 1260 |
| CAGGGGGACA GCGGAGAGTA TGCGTGCTCT GCGACCAACA ACATTGACAG CGTCCATGCC | 1320 |
| ACCGCTTTCA TCATCGTCCA GGCTCTTCCT CAGTTCACTG TGACGCCTCA GGACAGAGTC | 1380 |
| GTTATTGAGG GCCAGACCGT GGATTTCCAG TGTGAAGCCA AGGGCAACCC GCCGCCCGTC | 1440 |
| ATCGCCTGGA CCAAGGGAGG GAGCCAGCTC TCCGTGGACC GGCGGCACCT GGTCCTGTCA | 1500 |
| TCGGGAACAC TTAGAATCTC TGGTGTTGCC CTCCACGACC AGGGCCAGTA CGAATGCCAG | 1560 |
| GCTGTCAACA TCATCGGCTC CCAGAAGGTC GTGGCCCACC TGACTGTGCA GCCCAGAGTC | 1620 |
| ACCCCAGTGT TTGCCAGCAT TCCCAGCGAC ACAACAGTGG AGGTGGGCGC CAATGTGCAG | 1680 |
| CTCCCGTGCA GCTCCCAGGG CGAGCCCGAG CCAGCCATCA CCTGGAACAA GGATGGGGTT | 1740 |
| CAGGTGACAG AAAGTGGAAA ATTTCACATC AGCCCTGAAG GATTCTTGAC CATCAATGAC | 1800 |
| GTTGGCCCTG CAGACGCAGG TCGCTATGAG TGTGTGGCCC GGAACACCAT TGGGTCGGCC | 1860 |
| TCGGTGAGCA TGGTGCTCAG TGTGAACGTT CCTGACGTCA GTCGAAATGG AGATCCGTTT | 1920 |
| GTAGCTACCT CCATCGTGGA AGCGATTGCG ACTGTTGACA GAGCTATAAA CTCAACCCGA | 1980 |
| ACACATTTGT TTGACAGCCG TCCTCGTTCT CCAAATGATT TGCTGGCCTT GTTCCGGTAT | 2040 |
| CCGAGGGATC CTTACACAGT TGAACAGGCA CGGGCGGGAG AAATCTTTGA ACGGACATTG | 2100 |
| CAGCTCATTC AGGAGCATGT ACAGCATGGC TTGATGGTCG ACCTCAACGG AACAAGTTAC | 2160 |
| CACTACAACG ACCTGGTGTC TCCACAGTAC CTGAACCTCA TCGCAAACCT GTCGGGCTGT | 2220 |
| ACCGCCCACC GGCGCGTGAA CAACTGCTCG GACATGTGCT TCCACCAGAA GTACCGGACG | 2280 |
| CACGACGGCA CCTGTAACAA CCTGCAGCAC CCCATGTGGG GCGCCTCGCT GACCGCCTTC | 2340 |
| GAGCGCCTGC TGAAATCCGT GTACGAGAAT GGCTTCAACA CCCCTCGGGG CATCAACCCC | 2400 |
| CACCGACTGT ACAACGGGCA CGCCCTTCCC ATGCCGCGCC TGGTGTCCAC CACCCTGATC | 2460 |
| GGGACGGAGA CCGTCACACC CGACGAGCAG TTCACCCACA TGCTGATGCA GTGGGGCCAG | 2520 |
| TTCCTGGACC ACGACCTCGA CTCCACGGTG GTGGCCCTGA GCCAGGCACG CTTCTCCGAC | 2580 |
| GCACAGCACT GCAGCAACGT GTGCAGCAAC GACCCCCCCT GCTTCTCTGT CATGATCCCC | 2640 |
| CCCAATGACT CCCGGGCCAG GAGCGGGGCC CGCTGCATGT TCTTCGTGCG CTCCAGCCCT | 2700 |
| GTGTGCGGCA GCGGCATGAC TTCGCTGCTC ATGAACTCCG TGTACCCGCG GGAGCAGATC | 2760 |
| AACCAGCTCA CCTCCTACAT CGACGCATCC AACGTGTACG GGAGCACGGA GCATGAGGCC | 2820 |
| CGCAGCATCC GCGACCTGGC CAGCCACCGC GGCCTGCTGC GGCAGGGCAT CGTGCAGCGG | 2880 |
| TCCGGGAAGC CGCTGCTCCC CTTCGCCACC GGGCCGCCCA CGGAGTGCAT GCGGGACGAG | 2940 |
| AACGAGAGCC CCATCCCCTG CTTCCTGGCC GGGGACCACC GCGCCAACGA GCAGCTGGGC | 3000 |
| CTGACCAGCA TGCACACGCT GTGGTTCCGC GAGCACAACC GCATTGCCAC GGAGCTGCTC | 3060 |
| AAGCTGAACC CGCACTGGGA CGGCGACACC ATCTACTATG AGACCAGGAA GATCGTGGGT | 3120 |
| GCGGAGATCC AGCACATCAC CTACCAGCAC TGGCTCCCGA AGATCCTGGG GGAGGTGGGC | 3180 |
| ATGAGGACGC TGGGAGAGTA CCACGGCTAC GACCCCGGCA TCAATGCTGG CATCTTCAAC | 3240 |
| GCCTTCGCCA CCGCGGCCTT CAGGTTTGGC CACACGCTTG TCAACCCACT GCTTTACCGG | 3300 |
| CTGGACGAGA ACTTCCAGCC CATTGCACAA GATCACCTCC CCCTTCACAA AGCTTTCTTC | 3360 |
| TCTCCCTTCC GGATTGTGAA TGAGGGCGGC ATCGATCCGC TTCTCAGGGG GCTGTTCGGG | 3420 |
| GTGGCGCGGA AAATGCGTGT GCCCTCGCAG CTGCTGAACA CGGAGCTCAC GGAGCGGCTG | 3480 |
| TTCTCCATGG CACACACGGT GGCTCTGGAC CTGGCGGCCA TCAACATCCA GCGGCGCCGG | 3540 |
| GACCACGGGA TCCCACCCTA CCACGACTAC AGGGTCTACT GCAATCTATC GGCGGCACAC | 3600 |
| ACGTTCGAGG ACCTGAAAAA TGAGATTAAA AACCCTGAGA TCCGGGAGAA ACTGAAAAGG | 3660 |
| TTGTATGGCT CGACACTCAA CATCGACCTG TTTCCGGCGC TCGTGGTGGA GGACCTGGTG | 3720 |
| CCTGGCAGCC GGCTGGGCCC CACCCTGATG TGTCTTCTCA GCACACAGTT CAAGCGCCTG | 3780 |
| CGAGATGGGG ACAGGTTGTG GTATGAGAAC CCTGGGGTGT TCTCCCCGGC CCAGCTGACT | 3840 |
| CAGATCAAGC AGACGTCGCT GGCCAGGATC CTATGCGACA ACGCGGACAA CATCACCCGG | 3900 |
| GTGCAGAGCG ACGTGTTCAG GGTGGCGGAG TTCCCTCACG GCTACGGCAG CTGTGACGAG | 3960 |
| ATCCCCAGGG TGGACCTCCG GGTGTGGCAG GACTGCTGTG AAGACTGTAG GACCAGGGGG | 4020 |
| CAGTTCAATG CCTTTTCCTA TCATTTCCGA GGCAGACGGT CTCTTGAGTT CAGCTACCAG | 4080 |
| GAGGACAAGC CGACCAAGAA AACAAGACCA CGGAAAATAC CCAGTGTTGG GAGACAGGGG | 4140 |
| GAACATCTCA GCAACAGCAC CTCAGCCTTC AGCACACGCT CAGATGCATC TGGGACAAAT | 4200 |
| GACTTCAGAG AGTTTGTTCT GGAAATGCAG AAGACCATCA CAGACCTCAG AACACAGATA | 4260 |
| AAGAAACTTG AATCACGGCT CAGTACCACA GAGTGCGTGG ATGCCGGGGG CGAATCTCAC | 4320 |
| GCCAACAACA CCAAGTGGAA AAAAGATGCA TGCACCATTT GTGAATGCAA AGACGGGCAG | 4380 |
| GTCACCTGCT TCGTGGAAGC TTGCCCCCCT GCCACCTGTG CTGTCCCCGT GAACATCCCA | 4440 |
| GGGGCCTGCT GTCCAGTCTG CTTACAGAAG AGGGCGGAGG AAAACCCCTA GGCTCCTGGG | 4500 |
| AGGCTCCTCA GAGTTTGTCT GCTGTGCCAT CGTGAGATCG GGTGCCCGAT GGCAGGGAGC | 4560 |
| TGCGGACTGC AGACCAGGAA ACACCCAGAA CTCGTGACAT TTCATGACAA CGTCCAGCTG | 4620 |
| GTGCTGTTAC AGAAGGCAGT GCAGGAGGCT TCCAACCAGA GCATCTGCGG AGAAGGAGGC | 4680 |
| ACAGCAGGTG CCTGAAGGGA AGCAGGCAGG AGTCCTAGCT TCACGTTAGA CTTCTCAGGT | 4740 |
| TTTTATTTAA TTCTTTTAAA ATGAAAAATT GGTGCTACTA TTAAATTGCA CAGTTGAATC | 4800 |
| ATTTAGGCGC CTAAATTGGT TTTGCCTCCC AACACCATTT CTTTTTAAAT AAAGCAGGAT | 4860 |
| ACCTCTATAT GTCAGCCTTG CCTTGTTCAG ATGCCAGGAG CCGGCAGACC TGTCACCCGC | 4920 |
| AGGTGGGGTG AGTCTCGGAG CTGCCAGAGG GGCTCACCGA AATCGGGGTT CCATCACAAG | 4980 |
| CTATGTTTAA AAAGAAAATT GGTGTTTGGC AAACGGAACA GAACCTTTGA TGAGAGCGTT | 5040 |
| CACAGGGACA CTGTCTGGGG GTGCAGTGCA AGCCCCCGGC CTCTTCCCTG GGAACCTCTG | 5100 |
| AACTCCTCCT TCCTCTGGGC TCTCTGTAAC ATTTCACCAC ACGTCAGCAT CTAATCCCAA | 5160 |
| GACAAACATT CCCGCTGCTC GAAGCAGCTG TATAGCCTGT GACTCTCCGT GTGTCAGCTC | 5220 |
| CTTCCACACC TGATTAGAAC ATTCATAAGC CACATTTAGA AACAGATTTG CTTTCAGCTG | 5280 |
| TCACTTGCAC ACATACTGCC TAGTTGTGAA CCAAATGTGA AAAAACCTCC TTCATCCCAT | 5340 |
| TGTGTATCTG ATACCTGCCG AGGGCCAAGG GTGTGTGTTG ACAACGCCGC TCCCAGCCGG | 5400 |
| CCCTGGTTGC GTCCACGTCC TGAACAAGAG CCGCTTCCGG ATGGCTCTTC CCAAGGGAGG | 5460 |
| AGGAGCTCAA GTGTCGGGAA CTGTCTAACT TCAGGTTGTG TGAGTGCGTT | |

Seq ID NO: 87 Protein sequence:
Protein Accession #: BAA13219

| | |
|---|---|
| 1 11 21 31 41 51 | |
| \| \| \| \| \| \| | |
| SRPWWLRASE RPSAPSAMAK RSRGPGRRCL LALVLFCAWG TLAVVAQKPG AGCPSRCLCF | 60 |
| RTTVRCMHLL LEAVPAVAPQ TSILDLRFNR IREIQPGAFR RLRNLNTLLL NNNQIKRIPS | 120 |
| GAFEDLENLK YLYLYKNEIQ SIDRQAFKGL ASLEQLYLHF NQIETLDPDS FQHLPKLERL | 180 |
| FLHNNRITHL VPGTFNHLES MKRLRLDSWT LHCDCEILWL ADLLKTYAES GNAQAAAICE | 240 |
| YPRRIQGRSV ATITPEELNC ERPRITSEPQ DADVTSGNTV YFTCRAEGNP KPEIIWLRNN | 300 |
| NELSMKTDSR LNLLDDGTLM IQNTQETDQG IYQCMAKNVA GEVKTQEVTL RYFGSPARPT | 360 |
| FVIQPQNTEV LVGESVTLEC SATGHPPPRI SWTRGDRTPL PVDPRVNITP SGGLYIQNVV | 420 |
| QGDSGEYACS ATNNIDSVHA TAFIIVQALP QFTVTPQDRV VIEGQTVDFQ CEAKGNPPPV | 480 |
| IAWTKGGSQL SVDRRHLVLS SGTLRISGVA LHDQGQYECQ AVNIIGSQKV VAHLTVQPRV | 540 |
| TPVFASIPSD TTVEVGANVQ LPCSSQGEPE PAITWNKDGV QVTESGKFHI SPEGFLTIWD | 600 |
| VGPADAGRYE CVARNTIGSA SVSMVLSVNV PDVSRNGDPF VATSIVEAIA TVDRAINSTR | 660 |
| THLFDSRPRS PNDLLALFRY PRDPYTVEQA RAGEIFERTL QLIQEHVQHG LMVDLNGTSY | 720 |
| HYNDLVSPQY LNLTANLSGC TAHRRVNNCS DMCFHQKYRT HDGTCNNLQH PMWGASLTAF | 780 |
| ERLLKSVYEN GFNTPRGINP HRLYNGHALP MPRLVSTTLI GTETVTPDEQ FTHMLMQWGQ | 840 |
| FLDHDLDSTV VALSQARFSD GQHCSNVCSN DPPCFSVMIP PNDSRARSGA RCMFFVRSSP | 900 |
| VCGSGMTSLL MNSVYPREQI NQLTSYIDAS NVYGSTEHEA RSIRDLASHR GLLRQGIVQR | 960 |
| SGKPLLPFAT GPPTECMRDE NESPIPCFLA GDHRANEQLG LTSMHTLWFR EHNRIATELL | 1020 |
| KLNPHWDGDT IYYETRKIVG AETQHITYQH WLPKILGEVG MRTLGEYHGY DPGINAGIFN | 1080 |
| APATAAFRFG HTLVNPLLYR LDENFQPIAQ DHLPLHKAFF SPFRIVNEGG IDPLLRGLFG | 1140 |
| VAGKMRVPSQ LLNTELTERL FSMAHTVALD LAAINIQRGR DHGIPPYHDY RVYCNLSAAH | 1200 |
| TFEDLKNEIK NPEIREKLKR LYGSTLNIDL FPALVVEDLV PGSRLGPTLM CLLSTQFKRL | 1260 |
| RDGDRLWYEN PGVFSPAQLT QIKQTSLARI LCDNADNITR VQSDVFRVAE FPHGYGSCDE | 1320 |
| IPRVDLRVWQ DCCEDCRTRG QFNAFSYHFR GRRSLEFSYQ EDKPTKKTRP RKIPSVGRQG | 1380 |
| EHLSNSTSAF STRSDASGTN DFREFVLEMQ KTITDLRTQI KKLESRLSTT ECVDAGGESH | 1440 |
| ANNTKWKKDA CTICECKDGQ VTCFVEACPP ATCAVPVNIP GACCPVCLQK RAEEKP | |

Seq ID NO: 88 DNA sequence:
Nucleic Acid Accession #: NM_004834.1
Coding sequence: 80-3577 (underlined sequences correspond to
start and stop codons)

| | |
|---|---|
| 1 11 21 31 41 51 | |
| \| \| \| \| \| \| | |
| AATTCGAGGA TCCGGGTACC ATGGCACAGA GCGACAGAGA CATTTATTGT TATTTGTTTT | 60 |
| TTGGTGGCAA AAAGGGAAAA TGGGGAACGA CTCCCCTGCA AAAAGTCTGG TGGACATCGA | 120 |
| CCTCTCCTCC CTGCGGGATC CTGCTGGGAT TTTTGAGCTG GTGGAAGTGG TTGGAAATGG | 180 |
| CACCTATGGA CAAGTCTATA AGGGTCGACA TGTTAAAACG GGTCAGTTGG CAGCCATCAA | 240 |
| AGTTATGGAT GTCACTGAGG ATGAAGAGGA AGAAATCAAA CTGGAGATAA ATATGCTAAA | 300 |
| GAAATACTCT CATCACAGAA ACATTGCAAC ATATTATGGT GCTTTCATCA AAAAGAGCCC | 360 |
| TCCAGGACAT GATGACCAAC TCTGGCTTGT TATGGAGTTC TGTGGGGCTG GGTCCATTAC | 420 |
| AGACCTTGTG AAGAACACCA AAGGGAACAC ACTCAAAGAA GACTGGATCG CTTACATCTC | 480 |
| CAGAGAAATC CTGAGGGGAC TGGCACATCT TCACATTCAT CATGTGATTC ACCGGGATAT | 540 |
| CAAGGGCCAG AATGTGTTGC TGACTGAGAA TGCAGAGGTG AAACTTGTTG ACTTTGGTGT | 600 |
| GAGTGCTCAG CTGGACAGGA CTGTGGGGCG GAGAAATACG TTCATAGGCA CTCCCTACTG | 660 |
| GATGGCTCCT GAGGTCATCG CCTGTGATGA GAACCCAGAT GCCACCTATG ATTACAGAAG | 720 |
| TGATCTTTGG TCTTGTGGCA TTACAGCCAT TGAGATGGCA GAAGGTGCTC CCCCTCTCTG | 780 |
| TGACATGCAT CCAATGAGAG CACTGTTTCT CATTCCCAGA AACCCTCCTC CCCGGGTGAA | 840 |
| GTCAAAAAAA TGGTCGAAGA AGTTTTTTAG TTTTATAGAA GGGTGCCTGG TGAAGAATTA | 900 |
| CATGCAGCGG CCCTCTACAG AGCAGCTTTT GAAACATCCT TTTATAAGGG ATCAGCCAAA | 960 |
| TGAAAGGCAA GTTAGAATCC AGCTTAAGGA TCATATAGAT CGTACCAGGA AGAAGAGAGG | 1020 |
| CGAGAAAGAT GAAACTGAGT ATGAGTACAG TGGGAGTGAG GAAGAAGAGG AGGAAGTGCC | 1080 |
| TGAACAGGAA GGAGAGCCAA GTTCCATTGT GAACGTGCCT GGTGAGTCTA CTCTTCGCCG | 1140 |
| AGATTTCCTG AGACTGCAGC AGGAGAACAA GGAACGTTCC GAGGCTCTTC GGAGACAACA | 1200 |
| GTTACTACAG GAGCAACAGC TCCGGGAGCA GGAAGAATAT AAAAGGCAAC TGCTGGCAGA | 1260 |
| GAGACAGAAG CGGATTGAGC AGCAGAAAGA ACAGAGGCGA CGGCTAGAAG AGCAACAAAG | 1320 |
| GAGAGAGCGG GAGGCTAGAA GGCAGCAGGA ACGTGAACAG CGAAGGAGAG AACAAGAAGA | 1380 |
| AAAGAGGCGT CTAGAGGAGT TGGAGAGAAG GCGCAAAGAA GAAGAGGAGA GGAGACGGGC | 1440 |
| AGAAGAAGAA AAGAGGAGAG TTGAAAGAGA ACAGGAGTAT ATCAGGCGAC AGCTAGAAGA | 1500 |
| GGAGCAGCGG CACTTGGAAG TCCTTCAGCA GCAGCTGCTC CAGGAGCAGG CCATGTTACT | 1560 |
| GCATGACCAT AGGAGGCCGC ACCCGCAGCA CTCGCAGCAG CCGCCACCAC CGCAGCAGGA | 1620 |
| AAGGAGCAAG CCAAGCTTCC ATGCTCCCGA GCCCAAAGCC CACTACGAGC CTGCTGACCG | 1680 |
| AGCGCGAGAG GTTCCTGTGA GAACAACATC TCGCTCCCCT GTTCTGTCCC GTCGAGATTC | 1740 |
| CCCACTGCAG GGCAGTGGGC AGCAGAATAG CCAGGCAGGA CAGAGAAACT CCACCAGTAT | 1800 |
| TGAGCCCAGG CTTCTGTGGG AGAGAGTGGA GAAGCTGGTG CCCAGACCTG GCAGTGGCAG | 1860 |
| CTCCTCAGGG TCCAGCAACT CAGGATCCCA GCCCGGGTCT CACCCTGGGT CTCAGAGTGG | 1920 |
| CTCCGGGGAA CGCTTCAGAG TGAGATCATC ATCCAAGTCT GAAGGCTCTC CATCTCAGCG | 1980 |
| CCTGGAAAAT GCAGTGAAAA AACCTGAAGA TAAAAAGGAA GTTTTCAGAC CCCTCAAGCC | 2040 |
| TGCTGGCGAA GTGGATCTGA CCGCACTGGC CAAAGAGCTT CGAGCAGTGG AAGATGTACG | 2100 |
| GCCACCTCAC AAAGTAACGG ACTACTCCTC ATCCAGTGAG GAGTCGGGGA CGACGGATGA | 2160 |
| GGAGGACGAC GATGTGGAGC AGGAAGGGGC TGACGAGTCC ACCTCAGGAC CAGAGGACAC | 2220 |
| CAGAGCAGCG TCATCTCTGA ATTTGAGCAA TGGTGAAACG GAATCTGTGA AAACCATGAT | 2280 |
| TGTCCATGAT GATGTAGAAA GTGAGCCGGC CATGACCCCA TCCAAGGAGG GCACTCTAAT | 2340 |
| CGTCCGCCAG ACTCAGTCCG CTAGTAGCAC ACTCCAGAAA CACAAATCTT CCTCCTCCTT | 2400 |
| TACACCTTTT ATAGACCCCA GATTACTACA GATTTCTCCA TCTAGCGGAA CAACAGTGAC | 2460 |
| ATCTGTGGTG GGATTTTCCT GTGATGGGAT GAGACCAGAA GCCATAAGGC AAGATCCTAC | 2520 |
| CCGGAAAGGC TCAGTGGTCA ATGTGAATCC TACCAACACT AGGCCACAGA GTGACACCCC | 2580 |
| GGAGATTCGT AAATACAAGA AGAGGTTTAA CTCTGAGATT CTGTGTGCTG CCTTATGGGG | 2640 |
| AGTGAATTTG CTAGTGGGTA CAGAGAGTGG CCTGATGCTG CTGGACAGAA GTGGCCAAGG | 2700 |
| GAAGGTCTAT CCTCTTATCA ACCGAAGACG ATTTCAACAA ATGGACGTAC TTGAGGGCTT | 2760 |
| GAATGTCTTG GTGACAATAT CTGGCAAAAA GGATAAGTTA CGTGTCTACT ATTTGTCCTG | 2820 |
| GTTAAGAAAT AAAATACTTC ACAATGATCC AGAAGTTGAG AAGAAGCAGG GATGGACAAC | 2880 |
| CGTAGGGGAT TTGGAAGGAT GTGTACATTA TAAAGTTGTA AAATATGAAA GAATCAAATT | 2940 |
| TCTGGTGATT GCTTTGAAGA GTTCTGTGGA AGTCTATGCG TGGGCACCAA AGCCATATCA | 3000 |
| CAAATTTATG GCCTTTAAGT CATTTGGAGA ATTGGTACAT AAGCCATTAC TGGTGGATCT | 3060 |
| CACTGTTGAG GAAGGCCAGA GGTTGAAAGT GATCTATGGA TCCTGTGCTG GATTCCATGC | 3120 |
| TGTTGATGTG GATTCAGGAT CAGTCTATGA CATTTATCTA CCAACACATG TAAGAAAGAA | 3180 |
| CCCACACTCT ATGATCCAGT GTAGCATCAA ACCCCATGCA ATCATCATCC TCCCCAATAC | 3240 |
| AGATGGAATG GAGCTTCTGG TGTGCTATGA AGATGAGGGG GTTTATGTAA ACACATATGG | 3300 |
| AAGGATCACC AAGGATGTAG TTCTACAGTG GGGAGAGATG CCTACATCAG TAGCATATAT | 3360 |
| TCGATCCAAT CAGACAATGG GCTGGGGAGA GAAGGCCATA GAGATCCGAT CTGTGGAAAC | 3420 |
| TGGTCACTTG GATGGTGTGT TCATGCACAA AAGGGCTCAA AGACTAAAAT TCTTGTGTGA | 3480 |
| ACGCAATGAC AAGGTGTTCT TTGCCTCTGT TCGGTCTGGT GGCAGCAGTC AGGTTTATTT | 3540 |
| CATGACCTTA GGCAGGACTT CTCTTCTGAG CTGGTAGAAG CAGTGTGATC CAGGGATTAC | 3600 |
| TGGCCTCCAG AGTCTTCAAG ATCCTGAGAA CTTGGAATTC CTTGTAACTG GAGCTCGGAG | 3660 |
| CTGCACCGAG GGCAACCAGG ACAGCTGTGT GTGCAGACCT CATGTGTTGG GTTCTCTCCC | 3720 |
| CTCCTTCCTG TTCCTCTTAT ATACCAGTTT ATCCCCATTC TTTTTTTTTT TCTTACTCCA | 3780 |
| AAATAAATCA AGGCTGCAAT GCAGCTGGTG CTGTTCAGAT TCCAAAAAAA AAAAAAAACC | 3840 |
| ATGGTACCCG GATCCTCGAA TTCC | |

Seq ID No: 89 Protein sequence:
Protein Accession #: NP_004825.1

| | |
|---|---|
| 1 11 21 31 41 51 | |
| \| \| \| \| \| \| | |
| MANDSPAKSL TDIDLSSLRD PAGIFELVEV VGNGTYGQVY KCRHVKTGQL AAIKVMDVTE | 60 |
| DEEEEIKLEI NMLKKYSNHR NIATYYGAFI KKSPPGHDDQ LWLVMEFCGA GSITDLVKNT | 120 |
| KGNTLKEDWI AYISREILRG LAHLHIHHVI HRDIKGQNVL LTENAEVKLV DFGVSAQLDR | 180 |
| TVGRRNTFIG TPYWMAPEVI ACDENPDATY DYRSDLWSCG ITAIEMAEGA PPLCDMHPMR | 240 |
| ALFLIPRNPP PRLKSKKWSK KFFSFIEGCL VKNYMQRPST EQLLKHPFIR DQPNERQVRI | 300 |
| QLKDHIDRTR KKRGEKDETE YEYSGSEEEE EEVPEQEGEP SSIVNVPGES TLRRDFLRLQ | 360 |
| QENKERSEAL RRQQLLQEQQ LREQEEYKRQ LLAERQKRIE QQKEQRRRLE EQQRREREAR | 420 |
| RQQEREQRRR EQEEKRRLEE LERRRKEEEE RRRAEEEKRR VEREQEYIRR QLEEEQRHLE | 480 |
| VLQQQLLQEQ AMlLHDHRRP HPQHSQQPPP PQQERSKPSF HAPEPKAHYE PADRAREVPV | 540 |
| RTTSRSPVLS RRDSPLQGSG QQNSQAGQRN STSIEPRLLW ERVEKLVPRP GSGSSSCSSN | 600 |
| SGSQPGSHPG SQSGSGERFR VRSSSKSEGS PSQRLENAVK KPEDKKEVFR PLKPAGEVDL | 660 |
| TALAKELRAV EDVRPPHKVT DYSSSSEESC TTDEEDDDVE QEGADESTSG PEDTRAASSL | 720 |
| NLSNGETESV KTMIVHDDVE SEPANTPSKE GTLIVRQTQS ASSTLQKHKS SSSFTPFIDP | 780 |
| RLLQISPSSG TTVTSVVGFS CDGMRPEAIR QDPTRKGSVV NVNPTMTRPQ SDTPEIRKYK | 840 |
| KRFNSEILCA ALWGVMLLVG TESGLMLLDR SGQGKVYPLI NRRRFQQMDV LECLNVLVTI | 900 |
| SGKKDKLRVY YLSWLRNKIL HNDPEVEKKQ GWTTVGDLEG CVHYKVVKYE RIKFLVIALK | 960 |
| SSVEVYAWAP KPYEKFMAFK SFCELVHKPL LVDLTVEEGQ RLKVIYGSCA GFHAVDVDSG | 1020 |
| SVYDIYLPTH VRKMPHSMIQ CSIKPHAIII LPNTDGMELL VCYEDEGVYV NTYGRITKDV | 1080 |
| VLQWGEMPTS VAYIRSNQTM GWGEKAIEIR SVETGHLDGV FMHKRAQRLK FLCERNDKVF | 1140 |
| FASVRSGGSS QVYFMTLGRT SLLSW | |

Seq ID NO: 90 DNA sequence:
Nucleic Acid Accession #: none found
Coding sequence: 2-71 (underlined sequences correspond to start
and stop codons)

| | |
|---|---|
| 1 11 21 31 41 51 | |
| \| \| \| \| \| \| | |
| TTACACTTCA ATTCCTTACA CGGTATTTCA AACAAACAGT TTTGCTGAGA GGAGCTTTTG | 60 |
| TCTCTCCTTA10 AGAAAATGTT TATAAAGCTG AAAGGAAATC AAACAGTAAT CTTAAAAATG | 120 |
| AAAACAAAAC AACCCAACAA CCTAGATAAC TACAGTGATC AGGGAGCACA GTTCAACTCC | 180 |
| TTGTTATGTT TTAGTCATAT CGCCTACTCA AACAGCTAAA TAACAACACC AGTGGCAGAT | 240 |
| AAAAATCACC ATTTATCTTT CAGCTATTAA TCTTTTGAAT GAATAAACTG TGACAAACAA | 300 |
| ATTAACATTT TTGAACATGA AAGGCAACTT CTGCACAATC CTGTATCCAA GCAAACTTTA | 360 |
| AATTATCCAC TTAATTATTA CTTAATCTTA AAAAAAATTA GAACCCAGAA CTTTTCAATG | 420 |
| AAGCATTTGA AAGTTGAAGT GGAATTTAGG AAAGCCATAA AAATATAAAT ACTGTTATCA | 480 |
| CAGCACCAGC AAGCCATAAT CTTTATACCT ATCAGTTCTA TTTCTATTAA CAGTAAAAAC | 540 |
| ATTAAGCAAG ATATAAGACT ACCTGCCCAA GAATTCAGTC TTTTTTCATT TTTGTTTTTC | 600 |
| TCAGTTCTGA GGATCTTAAT CGTCAAATTT TCTTTGGACT GCATTCCTCA CTACTTTTTG | 660 |
| CACAATGGTC TCACGTTCTC ACATTTGTTC TCGCGAATAA ATTCATAAAA GGTGTTAAGT | 720 |
| TCTGTGAATG TCTTTTTAAT TATGGCCATA ATTGTGCTTG ACTGGATAAA AACTTAAGTC | 780 |
| CACCCTTATG TTTATAATAA TTTCTTGAGA ACAGCAAACT GCATTTACCA TCGTAAAACA | 840 |
| ACATCTGACT TACGGGAGCT GCAGGGAAGT GGTGAGACAG TTCGAACGGC TCCTCAGAAA | 900 |
| TCCAGTGACC CAATTCTAAA GACCATAGCA CCTGCAAGTG ACACAACAAG CAGATTTATT | 960 |
| ATACATTTAT TAGCCTTAGC AGGCAATAAA CCAAGAATCA CTTTGAAGAC ACAGCAAAAA | 1020 |
| GTGATACACT CCGCAGATCT GAAATAGATG TGTTCTCAGA CAACAAAGTC CCTTCAGAAT | 1080 |
| CTTCATGTTG CATAAATGTT ATGAATATTA ATAAAAAGTT GATTGAGA | |

Seq ID No: 91 Protein sequence:
Protein Accession #: none found

1 11 21 31 41 51
| | | | | |
YTSIPYTVFQ TNSFAERSFC LSL

Seq ID NO: 92 DNA sequence:
Nucleic Acid Accession #: NM_003706.1
Coding sequence: 310-1935 (underlined sequences correspond to start
and stop codons)

| | |
|---|---|
| 1 11 21 31 41 51 | |
| \| \| \| \| \| \| | |
| CACGAGGCAG GGGCCATTTT ACCTCCAGGT TGGCCCTGCT CAGGACCAGG AGGAAACACC | 60 |
| TCCAGGCCGC GACCTCCTCC CACAGGGGGA AAAGGAAAGC AGGAGGACCA CAGAAGCTTT | 120 |
| GGCACCGAGG ATCCCCGCAG TCTTCACCCG CGGAGATTCC GGCTGAAGGA GCTGTCCAGC | 180 |
| GACTACACCG CTAAGCGCAG GGAGCCCAAG CCTCCGCACC GGATTCCGGA GCACAAGCTC | 240 |
| CACCGCGCAT GCGCACACGC CCCAGACCCA GGCTCAGGAG GACTGAGAAT TTTCTGACCG | 300 |
| CAGTGCACCA TGGGAAGCTC TGAAGTTTCC ATAATTCCTG GGCTCCAGAA AGAAGAAAAG | 360 |
| GCGGCCGTGG AGAGACGAAG ACTTCATGTG CTGAAAGCTC TGAAGAAGCT AAGGATTGAG | 420 |
| GCTGATGAGG CCCCAGTTGT TGCTGTGCTG GGCTCAGGCG GAGGACTGCG GGCTCACATT | 480 |
| GCCTGCCTTG GGGTCCTGAG TGAGATGAAA GAACAGGGCC TGTTGGATGC CGTCACGTAC | 540 |
| CTCGCAGGGG TCTCTGGATC CACTTGGGCA ATATCTTCTC TCTACACCAA TGATGGTGAC | 600 |
| ATGGAAGCTC TCGAGGCTGA CCTGAAACAT CGATTTACCC GACAGGAGTG GGACTTGGCT | 660 |
| AAGAGCCTAC AGAAAACCAT CCAAGCAGCG AGGTCTGAGA ATTACTCTCT GACCGACTTC | 720 |
| TGGGCCTACA TGGTTATCTC TAAGCAAACC AGAGAACTGC CGGAGTCTCA TTTGTCCAAT | 780 |
| ATGAAGAAGC CCGTGGAAGA AGGGACACTA CCCTACCCAA TATTTGCAGC CATTGACAAT | 840 |
| GACCTGCAAC CTTCCTGGCA GGAGGCAAGA SCACCAGAGA CCTGGTTCGA GTTCACCCCT | 900 |
| CACCACGCTG GCTTCTCTGC ACTGGGGGCC TTTGTTTCCA TAACCCACTT CGGAAGCAAA | 960 |
| TTCAAGAAGG GAAGACTGGT CAGAACTCAC CCTGAGAGAG ACCTGACTTT CCTGAGAGGT | 1020 |
| TTATGGGGAA GTGCTCTTGG TAACACTGAA GTCATTAGGG AATACATTTT TGACCAGTTA | 1080 |
| AGGAATCTGA CCCTGAAAGG TTTATGGAGA AGGGCTGTTG CTAATGCTAA AAGCATTGGA | 1140 |
| CACCTTATTT TTGCCCGATT ACTGAGGCTG CAAGAAAGTT CACAAGGGGA ACATCCTCCC | 1200 |
| CCAGAAGATG AAGGCGGTGA GCCTGAACAC ACCTGGCTGA CTGAGATGCT CGAGAATTGG | 1260 |
| ACCAGGACCT CCCTGGAAAA GCAGGAGCAG CCCCATGAGG ACCCCGAAAG GAAAGGCTCA | 1320 |
| CTCAGTAACT TGATGGATTT TGTGAAGAAA ACAGGCATTT GCGCTTCAAA GTGGGAATGG | 1380 |
| GGGACCACTC ACAACTTCCT GTACAAACAC GGTGGCATCC GGGACAAGAT AATGAGCAGC | 1440 |
| CGGAAGCACC TCCACCTGGT GGATGCTGGT TTAGCCATCA ACACTCCCTT CCCACTCGTG | 1500 |
| CTGCCCCCGA CGCGGGAGGT TCACCTCATC CTCTCCTTCG ACTTCAGTGC CGGAGATCCT | 1560 |
| TTCGAGACCA TCCGGGCTAC CACTGACTAC TGCCGCCGCC ACAAGATCCC CTTTCCCCAA | 1620 |
| GTAGAAGAGG CTGAGCTGGA TTTGTGGTCC AAGGCCCCCG CCAGCTGCTA CATCCTGAAA | 1680 |
| GGAGAAACTG GACCAGTGGT GATACATTTT CCCCTGTTCA ACATAGATGC CTGTGGAGGT | 1740 |
| GATATTGAGG CATGGAGTGA CACATACGAC ACATTCAAGC TTGCTGACAC CTACACTCTA | 1800 |
| GATGTGGTGG TGCTACTCTT GGCATTAGCC AAGAAGAATG TCAGGGAAAA CAAGAAGAAG | 1860 |
| ATCCTTAGAG AGTTGATGAA CGTGGCCGGG CTCTACTACC CGAAGGATAG TGCCCGAAGT | 1920 |
| TGCTGCTTGG CATAGATGAG CCTCAGCTTC CAGGGCACTG TGGGCCTGTT GGTCTACTAG | 1980 |
| GGCCCTGAAG TCCACCTGGC CTTCCTGTTC TTCACTCCCT TCAGCCACAC GCTTCATGGC | 2040 |
| CTTGAGTTCA CCTTGGCTGT CCTAACAGGG CCAATCACCA GTGACCAGCT AGACTGTGAT | 2100 |
| TTTGATAGCG TCATTCAGAA GAAGGTGTCC AAGGAGCTGA AGGTGGTGAA ATTTGTCCTG | 2160 |
| CAGGTCCCTC GGGAGATCCT GGAGCTGGAG CATGAGTGTC TGACAATCAG AAGCATCATG | 2220 |
| TCCAATGTCC AGATGGCCAG AATGAATGTG ATAGTTCAGA CCAATGCCTT CCACTGCTCC | 2280 |
| TTTATGACTG CACTTCTAGC CAGTAGCTCT GCACAAGTTA GCTCTGTAGA AGTAAGAACT | 2340 |
| TGGGCTTAAA TCATGGGCTA TCTCTCCACA GCCAAGTGGA GCTCTGAGAA TACAACAAGT | 2400 |
| GCTCAATAAA TGCTTGCTGA TTGACTGATG AAAAAAAAAA AAAAAAAAAA AAAAAAAAAA | 2460 |
| AAAAAAAAAA AAAAAAAAAA AAAAAAAAAA AAAA | |

Seq ID No: 93 Protein sequence:
Protein Accession #: NP_003697.1

| | |
|---|---|
| 1 11 21 31 41 51 | |
| \| \| \| \| \| \| | |
| MGSSEVSIIP GLQKEEKAAV ERRRLHVLKA LKKLRIEADE APVVAVLGSG GGLRAHIACL | 60 |
| GVLSEMKEQG LLDAVTYLAG VSGSTWAISS LYTNDGDMEA LEADLKHRFT RQEWDLAKSL | 120 |
| QKTIQAARSE NYSLTDFWAY MVISKQTREL PESHLSNMKK PVEEGTLPYP IFAAIDNDLQ | 180 |
| PSWQEARAPE TWFEFTPHHA GFSALGAFVS ITHFGSKFKK GRLVRTHPER DLTFLRGLWG | 240 |
| SALGNTEVIR EYIFDQLRNL TLKGLWRRAV ANAKSIGHLI FARLLRLQES SQGEHPPPED | 300 |
| EGGEPEHTWL TEMLENWTRT SLEKQEQPHE DPERKGSLSN LMDFVKKTGI CASKWEWGTT | 360 |
| HNFLYKHGGI RDKIMSSRKH LHLVDAGLAI NTPFPLVLPP TREVHLILSF DFSAGDPFET | 420 |
| IRATTDYCRR HKIPFPQVEE AELDLWSKAP ASCYILKGET GPVVIHFPLF NIDACGGDIE | 480 |
| AWSDTYDTFK LADTYTLDVV VLLLALAKKN VRENKKKILR ELMNVAGLYY PKDSARSCCL | 540 |
| A | |

Seq ID NO: 94 DNA sequence:
Nucleic Acid Accession #: AK027351
Coding sequence: 1-642 (underlined sequences correspond to start and
stop codons)

| | |
|---|---|
| 1 11 21 31 41 51 | |
| \| \| \| \| \| \| | |
| AGGGAAAAAA ACTCCATTAA AAAGCCCAGC TTTCCTCCAT GTTAGATGTG ACTTGGAAAA | 60 |
| TGAGAAAGAT TTAGCAAAAT TCCACCGTAT CTTTTGCCAG GCTAGAGACA GGGAGAGCAG | 120 |
| AGTAAAACCC TCAGGCTGCT GAAATTTCTA GGCTGTTAGG AAGCCCCTCG AATTCTGTGA | 180 |
| AAATGAGGGT TTCTTAACTC ACACTGAGAG CGGAAAGGGG CAGACCCTTT TCATAACTCC | 240 |
| CTCAAGTGTG TGTTACCTTT CTTTACCAGC ATGGTAAGCA ACAGGACATA TCCCAGCCTC | 300 |
| GGACATGTCT GTATGATCCA AGGTACCCAA AGTCAGACAG AGTAAACTCA AGCCTGGCAC | 360 |
| TGGCTTTCTG CCGCTTCATG TGCTTTGGAA AAAGCAGGAG AAGCAATAGC AGCAGGAGTC | 420 |
| CCCAGCAGCT GGAGCCGCAA GAATGAACTG CAAAGAGGGA ACTGACAGCA GCTGCGGCTG | 480 |
| CAGGGGCAAC GACGAGAAGA AGATGTTGAA GTGTGTGGTG GTGGGGGACG GTGCCGTGGG | 540 |
| GAAAACCTGC CTGCTGATGA GCTACGCCAA CGACGCCTTC CCAGAGGAAT ACGTGCCCAC | 600 |
| TGTGTTTGAC CACTATGCAG TTACTGTGAC TGTGGGAGGC AAGCAACACT TGCTCGGACT | 660 |
| GTATGACACC GCGGGACAGG AGGACTACAA CCAGCTGAGG CCACTCTCCT ACCCCAACAC | 720 |
| GGATGTGTTT TTGATCTGCT TCTCTGTCGT AAACCCTGCC TCTTACCACA ATGTCCAGGA | 780 |
| GGAATGGGTC CCCGAGCTCA AGGACTGCAT GCCTCACGTG CCTTATGTCC TCATAGGGAC | 840 |
| CCAGATTGAT CTCCGTGATG ACCCAAAAAC CTTGGCCCGT TTGCTGTATA TGAAAGAGAA | 900 |
| ACCTCTCACT TACGAGCATG GTGTGAAGCT CGCAAAAGCG ATCGGAGCAC AGTGCTACTT | 960 |
| GGAATGTTCA GCTCTGACTC AGAAAGGTCT CAAAGCGGTT TTTGATGAAG CAATCCTCAC | 1020 |
| CATTTTCCAC CCCAAGAAAA AGAAGAAACG CTGTTCTGAG GGTCACAGCT GCTGTTCAAT | 1080 |
| TATCTGAGGT TGTCTGGGAC CTGCCTCCAC CCCATCCAGG GATGAGAATG GCAGCCAATC | 1140 |
| TCTGTGGCCA AGCTCCAGCC AAAAAGGAGG GCACGACCAG AAAGGAACTC CCTTTGCACG | 1200 |
| GAGGCTTGCC CCATCACCCT CTGAGCCCTC CCAACACAGC ACACTAGTCA GCCCACTGCC | 1260 |
| ACGACCTCCC TGCCAGCCAG AAGCATCCGT ACTGCACGCT GTCTGAGAAT GCTGGGCCTG | 1320 |
| GATTGCAGAC AGTGCCGCTG CTGATCGCAT CAAAAACAAA GTCAAAGGCC ATCTCACATT | 1380 |
| TTACAAATCC CCAGCTCATG AACGTGAAGC TGATAGGAAA TCACCCCAGG GAACCCGAAA | 1440 |
| AAGAAACTTG ATTCCTCTAT TGCTGGCCTT ACTTGATGTC TTTTATAAAA CTTGGGACTA | 1500 |
| CAATACTAAC CTTTTTTTCT GAATCTGCTG TTCTACCCAT GTGTCTCACA TTCATTTGTA | 1560 |
| TTATTTCAAG AAATGTACTA ATTTCCAGTT CACTCAGGCC TTACTAATCC ATACCAAATT | 1620 |
| AGCCTAAAGA CAAGGCATTT TATATTCATT TCTATTTTCA GCATGTTTCT ACCAAAGCTA | 1680 |
| TTAGAACCAA CACGTACCTC TGAATGCCCG ATTATAAGAA GACATGAGAA GACTTTAAAA | 1740 |
| GTTTTGGAAA TTTACAGAGC CATGATTTTT GAACCTAATT GAAAGAAAAC CATCTGAATT | 1800 |
| GTTGCAGGTC CACATTTTTG CCAAAGATAC ACTCTATAGA TGCTTAGTAG TCGCCTGATT | 1860 |
| TTTTTCCATG TATTGCCACG ACAAACTAAA AATGAACTGT GTTTAAGAAT GTAGTATTTC | 1920 |
| TGTTTTTCAT CCAAGTTGAT TGGGGGAAGA ATATGGCAGG ATCCATCTTT TACAGTATTT | 1980 |
| TGTATTCAGT AAAGTGGACA TTCCTGCTCC TCCCTTCCCC CATTGCATGC CCTCTTCCTC | 2040 |
| CCTTGATTTC ACTTTCTCTC ATGCCCGGAT CCTTTTATTC TCCCCAGTTA TAACCCAGTT | 2100 |
| ATAAAAGAAA GATCTGAGCA TAAAGATACG TGTTTAAAAA TAACTAAAAG TAAAGGAAAG | 2160 |
| TGCCTTAATT TTTCTATTTG CTTCAACTGA AAGTGCTTCT CAGCTCGCCC CATGTAAGTT | 2220 |
| CTCATTCCAT GTAAATGACA TTTTCCAGTT ACAACTGGTA CTGAGATTTT GCCTCTCTCT | 2280 |
| TTCCTTACTC ATCCTCCCAA ATGTCTTTGT GGGAGCCATA TCAGTGGATA CCAAGCTCTG | 2340 |
| TATGCATTTG TCCCCTGCCC TCCACAATGT GTGACATAGA ACAGGGACTT TGGCCCTGGG | 2400 |
| AAAGCAAAAG CTCCCAGTAA GGAATCCTGT GCCCAATGAT GTAAAACAAT TCCAAACATC | 2460 |
| CAGGAATTTT TGTATCATAG AGCGAATTAC TTCCTATCTT TTCATTAGAG GCTATGAGGA | 2520 |
| CTTCTAATTA GTCTTAGTTG CTTATAAGTG CCCTGGAATC ACCCAGGTAG GCACTTAATT | 2580 |
| TTTTTTTCAG TTGCATGAGC AAAGTGCTTC TTAGTAGTGT GAAATTACAA CAACTTTAAG | 2640 |
| ACTTTCCAGA TTCAAGCTCC CACTGTTGGA AAAAGCCAGC CTTTCTAATC TCTTCTGCTA | 2700 |
| CTGGAATAAG CACTTAAGAA TTGCGTGATA GCCAGGCACC GTGGCTCATG CCTGTAATCC | 2760 |
| CAACACTTAG GGAGGCTGAG GTGGGTGGGC CGCTTGAGCT CAGGAGTTCA AGACCAGCCT | 2820 |
| GGGTAATATA GTGAGATCCT GTGTCTCTAT AAAAAAATTA AAAATTAGTC AGTTGTAGTG | 2880 |
| ACACATACCT GTAGTCCCAG CTACTCAGGA GGCTGAGGTG GAAGGATCAC TTGAGCCCAG | 2940 |
| AAGGTAAGGC TGCAGTGAGC TGTGACTGTG CCACTACACT CCAGCCTGAG TGACAGAGAA | 3000 |
| AGAACCTGTC AAAAAAAAAA AAAAAACAAC CTACATTTCA AGTACTATTT CCCTTCTCTC | 3060 |
| CCATCTAATT GCTAAAGATT TTCTTTCATA CGCACACACT CCAGTGACTG GAAAAACGGG | 3120 |
| AGTTTTCAGT CAAAGCTTGA CATTTAGAGA AAACAAGGAC TTTCTGCCTT TATAAATGGA | 3180 |
| AATCAACTGT GTATGAACTA TAACTCTGCA GAGGTTATGA ATTCATCCTT TACAAACAAT | 3240 |
| AATGAACTTT TAGTCCTGTA ATAAATGAAA TGTTATTAGG CAGCTTTGTT GCATGATTGC | 3300 |
| ATAGTTATAT CTTGCTAACG GGCCACTCAT TTCTCACTGA TGTGGATGAA AAAATGAGAG | 3360 |
| CAGTATGTTT CCAGGTGTGT GCACTCAACA GGCAAATAGC TCCCGAGGTC ACCACTTCCC | 3420 |
| TAATGGGCCA CAGGAAGTAA GTTGATCTTG ATGGGGAGAT CACGTCACCC AGAACCAGCA | 3480 |
| ACTGGATAGA GACTGTTGTT AGTGTCTGGG TAGAGCACAG GCTCCCAGGG GTCTTAAGAG | 3540 |
| CTAATTACTG AATAAAACAA TCTAGAACAA AGCAA | |

Seq ID No: 95 Protein sequence:
Protein Accession #: CAC06611.1

| | |
|---|---|
| 1 11 21 31 41 51 | |
| \| \| \| \| \| \| | |
| MNCKEGTDSS CGCRGNDEKK MLKCVVVGDG AVGKTCLLMS YANDAFPEEY VPTVFDHYAV | 60 |
| TVTVGGKQHL LGLYDTAGQE DYNQLRPLSY PNTDVFLICF SVVNPASYHN VQEEWVPELK | 120 |
| DCMPHVPYVL IGTQIDLRDD PKTLARLLYM KEKPLTYEHG VKLAKAIGAQ CYLECSALTQ | 180 |
| KGLKAVFDEA ILTIFHPKKK KKRCSEGHSC CSII | |

Seq ID NO: 96 DNA sequence:
Nucleic Acid Accession #: NM_003654.1
Coding sequence: 367-1602 (underlined sequences correspond to start and stop codons)

| | |
|---|---|
| 1 11 21 31 41 51 | |
| \| \| \| \| \| \| | |
| GGGGAGGGCG CGGGAGGCGG AGGATGCCGC CGCGGCTGCT GCCGCCGCCG CCACCCGCGG | 60 |
| GTCCCCGGCG ACCCTACTCC AGACCCGAGG ATGGAGCCGG CGCTGGGCGC TGCAGCTGCT | 120 |
| CCCGGCGCGT CCCCGACCAG GTAGCTGGTG TCACTTCGGT GTGGTTGGAA GAAGACTTTC | 180 |
| TCCCCAGCTG CATTCCCGGA GGCGCCCTTT CGACCTGGAG GCCGGGTCTG CTGGCCACAG | 240 |
| GGCTGCCGCA CTGGCTGGGA CTGCCAGCTG GGCCTGGAGA CGCTGGTGGC TGTGGACTCC | 300 |
| CCAGCTTGGA GCAGTCCCTC TTTGACCTCA CCCCTTGCAG AAGCAGCCCC ATGAAGGTGC | 360 |
| CCAGCCATGC AATGTTCCTG GAAGGCCGTC CTCCTCCTTG CCCTGGCCTC CATTGCCATC | 420 |
| CAGTACACGG CCATCCGCAC CTTCACCGCC AAGTCCTTTC ACACCTGCCC CGGGCTGGCA | 480 |
| GAGGCCGGGC TGGCCGAGCG ACTGTGCGAG GAGAGCCCCA CCTTCGCCTA CAACCTCTCC | 540 |
| CGCAAGACCC ACATCCTCAT CCTGGCCACC ACGCGCAGCG GCTCCTCCTT CGTGGGCCAG | 600 |
| CTCTTCAACC AGCACCTGGA CGTCTTCTAC CTGTTTGAGC CCCTCTACCA CGTCCAGAAC | 660 |
| ACGCTCATCC CCCGCTTCAC CCAGGGCAAG AGCCCGGCCG ACCGGCGGGT CATGCTAGGC | 720 |
| GCCAGCCGCG ACCTCCTGCG GAGCCTCTAC GACTGCGACC TCTACTTCCT GGAGAACTAC | 780 |
| ATCAAGCCGC CGCCGGTCAA CCACACCACC GACAGGATCT TCCGCCGCGG GGCCAGCCGG | 840 |
| GTCCTCTGCT CCCGGCCTGT GTGCGACCCT CCGGGGCCAG CCGACCTGGT CCTGGAGGAG | 900 |
| GGGGACTGTG TGCGCAAGTG CGGGCTACTC AACCTGACCG TGGCGGCCGA GGCGTGCCGC | 960 |
| GAGCGCAGCC ACGTGGCCAT CAAGACGGTG CGCGTGCCCG AGGTGAACGA CCTGCGCGCC | 1020 |
| CTGGTGGAAG ACCCGCGATT AAACCTCAAG GTCATCCAGC TGGTCCGAGA CCCCCGCGGC | 1080 |
| ATTCTGGCTT CGCGCAGCGA GACCTTCCGC GACACGTACC GGCTCTGGCG GCTCTGGTAC | 1140 |
| GGCACCGGGA GGAAACCCTA CAACCTGGAC GTGACGCAGC TGACCACGGT GTGCGAGGAC | 1200 |
| TTCTCCAACT CCGTGTCCAC CGGCCTCATG CGGCCCCCGT GGCTCAAGGG CAAGTACATG | 1260 |
| TTGGTGCGCT ACGAGGACCT GGCTCGGAAC CCTATGAAGA AGACCGAGGA GATCTACGGG | 1320 |
| TTCCTGGGCA TCCCGCTGGA CAGCCACGTG GCCCGCTGGA TCCAGAACAA CACGCGGGGC | 1380 |
| GACCCCACCC TGGGCAAGCA CAAATACGGC ACCGTGCGAA ACTCGGCGGC CACGGCCGAG | 1440 |
| AAGTGGCGCT TCCGCCTCTC CTACGACATC GTGGCCTTTG CCCAGAACGC CTGCCAGCAG | 1500 |
| GTGCTGGCCC AGCTGGGCTA CAAGATCGCC GCCTCGGAGG AGGAGCTGAA GAACCCCTCG | 1560 |
| GTCAGCCTGG TGGAGGAGCG GGACTTCCGC CCCTTCTCGT GACCCGGGCG GTGCGGGTGG | 1620 |
| GGGCGGGAGG CGCAAGGTGT CGGTTTTGAT AAAATGGACC GTTTTTAACT GTTGCCTTAT | 1680 |
| TAACCCCTCC CTCTCCCACC TCATCTTCGT GTCCTTCCTG CCCCCAGCTC ACCCCACTCC | 1740 |
| CTTCTGCCCC TTTTTTGTCT CTGAAATTTG CACTACGTCT TGGACGGGAA TCACTGGGGC | 1800 |
| AGAGGGCGCC TGAAGTAGGG TCCCGCCCCC CCCACCCCAT TCAGACACAT GGATGTTGGG | 1860 |
| TCTCTGTGCG GACGGTGACA ATGTTTACAA GCACCACATT TACACATCCA CACACGCACA | 1920 |
| CGGGCACTCG CGAGGCGACT TCTCAAGCTT TTGAATGGGT GAGTGGTCGG GTATCTAGTT | 1980 |
| TTTGCACTGT CTTACTATTC AAGGTAAGAG GATACAAACA AGAGGACCAC TTGTCTCTAA | 2040 |
| TTTATGAATG GTGTCCATCC TTTCCCCATC CCTGCCTCCT GCCCCTGACG CCCATTTCCC | 2100 |
| CCCTTAGAGC AGCGAAACTG CCCCCTCCTG CCCGCCCTTG CCTGTCGGTG AGGCAGGTTT | 2160 |
| TTACTGTGAG GTGAACGTGG ACCTGTTTCT GTTTCCAGTC TGTGGTGATG CTGTCTGTCT | 2220 |
| GTCTGAGTCT CGTGGCCGCC CCTGGACCAG TGATGACTGA TGAATCTTAT GAGCTTCTGA | 2280 |
| TTGATCTCGG GGTCCATCTG TGATATTTCT TTGTGCCAAA AAGAAAAAAA AAGAGTGGAT | 2340 |
| CAGTTTGCTA AATGAACATT GAAATTGAAA TGCTTTATCT GTGTTTTCTG TAAATAAAAG | 2400 |
| AGTGCAATAA TCACC | |

Seq ID No: 97 Protein sequence:
Protein Accession #: NP_003645.1

| | |
|---|---|
| 1 11 21 31 41 51 | |
| \| \| \| \| \| \| | |
| MQCSWKAVLL LALASIAIQY TAIRTFTAKS FHTCPGLAEA GLAERLCEES PTFAYNLSRK | 60 |
| THILILATTR SGSSFVGQLF NQHLDVFYLF EPLYHVQNTL IPRFTQGKSP ADRRVMLGAS | 120 |
| RDLLRSLYDC DLYFLENYIK PPPVNHTTDR IFRRGASRVL CSRPVCDPPG PADLVLEEGD | 180 |
| CVRKCGLLNL TVAAEACRER SHVAIKTVRV PEVNDLRALV EDPRLNLKVI QLVRDPRGIL | 240 |
| ASRSETFRDT YRLWRLWYGT GRKPYNLDVT QLTTVCEDFS NSVSTGLMRP PWLKGKYMLV | 300 |
| RYEDLARNPM KKTEEIYGFL GIPLGSHYAR WIQNNTRGDP TLGKHKYGTV RNSAATAEKW | 360 |
| RFRLSYDIVA FAQNACQQVL AQLGYKIAAS EEELKNPSVS LVEERDFRPF S | |

Seq ID NO: 98 DNA sequence:
Nucleic Acid Accession #: NM_002852.1
Coding sequence: 68-1213 (underlined sequences correspond to start
and stop codons)

| | |
|---|---|
| 1 11 21 31 41 51 | |
| \| \| \| \| \| \| | |
| CTCAAACTCA GCTCACTTGA GAGTCTCCTC CCGCCAGCTG TGGAAAGAAC TTTGCGTCTC | 60 |
| TCCAGCAATG CATCTCCTTG CGATTCTGTT TTGTGCTCTC TGGTCTGCAG TGTTGGCCGA | 120 |
| GAACTCGGAT GATTATGATC TCATGTATGT GAATTTGGAC AACGAAATAG ACAATGGACT | 180 |
| CCATCCCACT GAGGACCCCA CGCCGTGCGA CTGCGGTCAG GAGCACTCGG AATGGGACAA | 240 |
| GCTCTTCATC ATGCTGGAGA ACTCGCAGAT GAGAGAGCGC ATGCTGCTGC AAGCCACGGA | 300 |
| CGACGTCCTG CGGGGCGAGC TGCAGAGGCT GCGGGAGGAG CTGGGCCGGC TCGCGGAAAG | 360 |
| CCTGGCGAGG CCGTGCGCGC CGGGGGCTCC CGCAGAGGCC AGGCTGACCA GTGCTCTGGA | 420 |
| CGAGCTGCTG CAGGCGACCC GCGACGCGGG CCGCAGGCTG GCGCGTATGG AGGGCGCGGA | 480 |
| GGCGCAGCGC CCAGAGGAGG CGGGGCGCGC CCTGGCCGCG GTGCTAGAGG AGCTGCGGCA | 540 |
| GACGCGAGCC GACCTGCACG CGGTGCAGGG CTGGGCTGCC CGGAGCTGGC TGCCGGCAGG | 600 |
| TTGTGAAACA GCTATTTTAT TCCCAATGCG TTCCAAGAAG ATTTTTGGAA GCGTGCATCC | 660 |
| AGTGAGACCA ATGAGGCTTG AGTCTTTTAG TGCCTGCATT TGGGTCAAAG CCACAGATGT | 720 |
| ATTAAACAAA ACCATCCTGT TTTCCTATGG CACAAAGAGG AATCCATATG AAATCCAGCT | 780 |
| GTATCTCAGC TACCAATCCA TAGTGTTTGT GGTGGGTGGA GAGGAGAACA AACTGGTTGC | 840 |
| TGAAGCCATG GTTTCCCTGG GAAGGTGGAC CCACCTGTGC GGCACCTGGA ATTCAGAGGA | 900 |
| AGGGCTCACA TCCTTGTGGG TAAATGGTGA ACTGGCGGCT ACCACTGTTG AGATGGCCAC | 960 |
| AGGTCACATT GTTCCTGAGG GAGGAATCCT GCAGATTGGC CAAGAAAAGA ATGGCTGCTG | 1020 |
| TGTGGGTGGT GGCTTTGATG AAACATTAGC CTTCTCTGGG AGACTCACAG GCTTCAATAT | 1080 |
| CTGGGATAGT GTTCTTAGCA ATGAAGAGAT AAGAGAGACC GGAGGAGCAG AGTCTTGTCA | 1140 |
| CATCCGGGGG AATATTGTTG GGTGGGGAGT CACAGAGATC CAGCCACATG GAGGAGCTCA | 1200 |
| GTATGTTTCA TAAATGTTGT GAAACTCCAC TTGAAGCCAA AGAAAGAAAC TCACACTTAA | 1260 |
| AACACATGCC AGTTGGGGAG GTCTGAAAAC TCAGTGCATA ATAGGAACAC TTGAGACTAA | 1320 |
| TGAAAGAGAG AGTTGAGACC AATCTTTATT TGTACTGGCC AAATACTGAA TAAACAGTTG | 1380 |
| AAGGAAAGAC ATTGGAAAAA GCTTTTGAGG ATAATGTTAC TAGACTTTAT GCCATGGTGC | 1440 |
| TTTCAGTTTA ATGCTGTGTC TCTGTCAGAT AAACTCTCAA ATAATTAAAA AGGACTGTAT | 1500 |
| TGTTGAACAG AGGGACAATT GTTTTACTTT TCTTTGGTTA ATTTTGTTTT GGCCAGAGAT | 1560 |
| GAATTTTACA TTGGAAGAAT AACAAAATAA GATTTGTTGT CCATTGTTCA TTGTTATTGG | 1620 |
| TATGTACCTT ATTACAAAAA AAATGATCAA AACATATTTA TACTACAAGG TGACTTAACA | 1680 |
| ACTATAAATG TAGTTTATGT GTTATAATCG AATGTCACGT TTTTGAGAAG ATAGTCATAT | 1740 |
| AAGTTATATT GCAAAAGGGA TTTGTATTAA TTTAAGACTA TTTTTGTAAA GCTCTACTGT | 1800 |
| AAATAAAATA TTTTATAAAA CTAAAAAAAA AAAAAAA | |

Seq ID No: 99 Protein sequence:
Protein Accession #: NP_002843.1

| | |
|---|---|
| 1 11 21 31 41 51 | |
| \| \| \| \| \| \| | |
| MHLLAILFCA LWSAVLAENS DDYDLMYVNL DNEIDNGLHP TEDPTPCDCG QEESEWDKLF | 60 |
| IMLENSQMRE RMLLQATDDV LRGELQRLRE ELGRLAESLA RPCAPGAPAE ARLTSALDEL | 120 |
| LQATRDAGRR LARMEGAEAQ RPEEAGRALA AVLEELRQTR ADLHAVQGWA ARSWLPAGCE | 180 |
| TAILFPMRSK KIFGSVHPVR PMRLESFSAC IWVKATDVLN KTILFSYGTK RNPYEIQLYL | 240 |
| SYQSIVFVVG GEENKLVAEA MVSLGRWTHL CGTWNSEEGL TSLWVNCELA ATTVEMATGH | 300 |
| IVPEGGILQI GQEKNGCCVG GGFDETLAFS CRLTGFNIWD SVLSNEEIRE TGGAESCHIR | 360 |
| GNIVGWCVTE IQPHGGAQYV S | |

Seq ID NO: 100 DNA sequence:
Nucleic Acid Accession #: NM_007351.1
Coding sequence: 72-3758 (underlined sequences correspond to start and
stop codons)

| | |
|---|---|
| 1 11 21 31 41 51 | |
| \| \| \| \| \| \| | |
| CTGCTATCAA AAAGGCCATA AGGATTTTGT CCCCAAATTT CACATGAGCT ACCTTGCTTC | 60 |
| AAACTACTGA GATGAAGGGG GCAAGATTAT TTGTCCTTCT TTCTAGTTTA TGGAGTGGGG | 120 |
| GCATTGGGCT TAACAACAGT AAGCATTCTT GGACTATACC TGAGGATGGG AACTCTCAGA | 180 |
| AGACTATGCC TTCTGCTTCA GTTCCTCCAA ATAAAATACA AAGTTTGCAA ATACTGCCAA | 240 |
| CCACTCGGGT CATGTCGGCG GAGATAGCTA CAACTCCAGA GGCAAGAACT TCTGAAGACA | 300 |
| GTCTTCTTAA ATCAACACTG CCTCCCTCAG AAACAAGTGC ACCTGCTGAG GGTGTGAGAA | 360 |
| ATCAAACTCT CACATCCACA GAGAAAGCAG AAGGAGTGGT CAAGTTACAG AATCTTACCC | 420 |
| TCCCAACCAA CGCTAGCATC AAGTTCAATC CTGGAGCAGA ATCAGTGGTC CTTTCCAATT | 480 |
| CTACACTGAA ATTTCTTCAG AGCTTTGCCA GAAAGTCAAA TGAACAAGCA ACTTCTCTAA | 540 |
| ACACAGTTGG AGGCACTGGA GGCATTGGAG GCGTTGGAGG CACTGGAGGC GTGGGAAATC | 600 |
| GAGCCCCACG GGAAACATAC CTCAGCCGGG GTGACAGCAG TTCCAGCCAA AGAACTGACT | 660 |
| ACCAAAAATC AAATTTCGAA ACAACTAGAG GAAAGAATTG GTGTGCTTAT GTACATACCA | 720 |
| CGTTATCTCC CACAGTGACA TTGGACAACC AGGTCACTTA TGTCCCAGGT GGGAAAGGAC | 780 |
| CTTGTGGCTG GACCGGTGGA TCCTGTCCTC AGAGATCTCA GAAGATATCC AATCCTGTCT | 840 |
| ATAGGATGCA ACATAAAATT GTCACCTCAT TGGATTGGAG GTGCTGTCCT GGATACAGTG | 900 |
| GGCCGAAATG TCAACTAAGA GCCCAGGAAC AGCAAAGTTT GATACACACC AACCAGGCTG | 960 |
| AAAGTCATAC AGCTGTTGGC AGAGGAGTAG CTGAGCAGCA GCAGCAGCAA GGCTGTGGTG | 1020 |
| ACCCAGAAGT GATGCAAAAA ATGACTGATC AGGTGAACTA CCAGGCAATG AAACTGACTC | 1080 |
| TTCTGCAGAA GAAGATTGAC AATATTTCTT TGACTGTGAA TGATGTAAGG AACACTTACT | 1140 |
| CCTCCCTAGA AGGAAAAGTC AGCGAAGATA AAAGCAGAGA ATTTCAATCT CTTCTAAAAG | 1200 |
| GTCTAAAATC CAAAAGCATT AATGTACTGA TAAGAGACAT AGTAAGAGAA CAATTTAAAA | 1260 |
| TTTTTCAAAA TGACATGCAA GAGACTGTAG CACAGCTCTT CAAGACTGTA TCAAGTCTAT | 1320 |
| CAGAGGACCT CGAAAGCACC AGGCAAATAA TTCAAAAAGT TAATGAATCT GTGGTTTCAA | 1380 |
| TAGCAGCCCA GCAAAAGTTT GTTTTGGTGC AAGAGAATCG GCCCACTTTG ACTGATATAG | 1440 |
| TGGAACTAAG GAATCACATT GTGAATGTAA GGCAAGAAAT GACTCTTACA TGTGAGAAGC | 1500 |
| CTATTAAAGA ACTAGAAGTA AAGCAGACTC ATTTAGAAGG TGCTCTAGAA CAGGAACACT | 1560 |
| CAAGAAGCAT TCTGTATTAT GAATCCCTCA ATAAAACTCT TTCTAAATTG AAGGAAGTAC | 1620 |
| ATGAGCAGCT TTTATCAACT GAACAGGTAT CAGACCAGAA GAATGCTCCA GCTGCTGAGT | 1680 |
| CAGTTAGCAA TAATGTCACT GAGTACATGT CTACTTTACA TGAAAATATA AAGAAGCAGA | 1740 |
| GTTTGATGAT GCTGCAAATG TTTGAAGATT TGCACATTCA AGAAAGCAAG ATTAACAATC | 1800 |
| TCACCGTCTC TTTGGAGATG GAGAAAGAGT CTCTCAGAGG TGAATGTGAA GACATGTTAT | 1860 |
| CCAAATGCAG AAATGATTTT AAATTTCAAC TTAAGGACAC AGAAGAGAAT TTACATGTGT | 1920 |
| TAAATCAAAC ATTGGCTGAA GTTCTCTTTC CAATGGACAA TAAGATGGAC AAAATGAGTG | 1980 |
| AGCAACTAAA TGATTTGACT TATGATATGG AGATCCTTCA ACCCTTGCTT GAGCAGGGAG | 2040 |
| CATCACTCAG ACAGACAATG ACATATGAAC AACCAAAGGA AGCAATAGTG ATAAGGAAAA | 2100 |
| AGATAGAAAA TCTGACTAGT GCTGTCAATA GTCTAAATTT TATTATCAAA GAACTTACAA | 2160 |
| AAAGACACAA CTTACTTAGA AATGAAGTAC AGGGTCGTGA TGATGCCTTA GAAAGACGTA | 2220 |
| TCAATGAATA TGCCTTAGAA ATGGAAGATG GCCTCAATAA GACAATGACT ATTATAAATA | 2280 |
| ATGCTATTGA TTTCATTCAA GATAACTATG CCCTAAAAGA GACTTTAAGT ACTATTAAGG | 2340 |
| ATAATAGTGA GATCCATCAT AAATGTACCT CCGATATGGA AACTATTTTG ACATTTATTC | 2400 |
| CTCAGTTCCA CCGTCTGAAT GATTCTATTC AGACTTTGGT CAATGACAAT CAGAGATATA | 2460 |
| ACTTTGTTTT GCAAGTCGCC AAGACCCTTG CAGGTATTCC CAGAGATGAG AAACTAAATC | 2520 |
| AGTCCAACTT CCAAAAGATG TATCAAATGT TCAATGAAAC CACTTCCCAA GTGAGAAAAT | 2580 |
| ACCAGCAAAA TATGAGTCAT TTGGAAGAAA AACTACTCTT AACTACCAAG ATTTCCAAAA | 2640 |
| ATTTTGAGAC TCGGTTGCAA GACATTGAGT CTAAAGTTAC CCAGACGCTC ATACCTTATT | 2700 |
| ATATTTCAGT TAAAAAAGGC AGTGTAGTTA CAAATGAGAG AGATCAGGCT CTTCAACTGC | 2760 |
| AAGTATTAAA TTCCAGATTT AAGGCGTTGG AAGCAAAATC TATCCATCTT TCAATTAACT | 2820 |
| TCTTTTCGCT TAACAAAACT CTCCACGAAG TTTTAACAAT GTGTCACAAT GCTTCTACAA | 2880 |
| GTGTGTCAGA ACTGAATGCT ACCATCCCTA AGTGGATAAA ACATTCCCTG CCAGATATTC | 2940 |
| AACTTCTTCA GAAAGGTCTA ACAGAATTTG TGGAACCAAT AATTCAAATA AAAACTCAAG | 3000 |
| CTGCCCTATC TAATTCAACT TGTTGTATAG ATCGATCGTT GCCTGGTAGT CTGGCAAATG | 3060 |
| TTGTCAAGTC TCAGAAGCAA GTAAAATCAT TGCCAAAGAA AATTAACGCA CTTAAGAAAC | 3120 |
| CAACGGTAAA TCTTACCACA GTCCTGATAG GCCGGACTCA AAGAAACACG GACAACATAA | 3180 |
| TATATCCTGA GGAGTATTCA AGCTGTAGTC GGCATCCGTG CCAAAATGGG GGCACGTGCA | 3240 |
| TAAATGGAAG AACTAGCTTT ACCTGTGCCT GCAGACATCC TTTTACTGGT GACAACTGCA | 3300 |
| CTATCAAGCT TGTGGAAGAA AATGCTTTAG CTCCAGATTT TTCCAAAGGA TCTTACAGAT | 3360 |
| ATGCACCCAT GGTGGCATTT TTTGCATCTC ATACGTATGG AATGACTATA CCTGGTCCTA | 3420 |
| TCCTGTTTAA TAACTTGGAT GTCAATTATG GAGCTTCATA TACCCCAAGA ACTGGAAAAT | 3480 |
| TTAGAATTCC GTATCTTGGA GTATATGTTT TCAAGTACAC CATCGAGTCA TTTAGTGCTC | 3540 |
| ATATTTCTGG ATTTTTAGTG GTTGATGGAA TAGACAAGCT TGCATTTGAG TCTGAAAATA | 3600 |
| TTAACAGTGA AATACACTGT GATAGGGTTT TAACTGGGGA TGCCTTATTA GAATTAAATT | 3660 |
| ATGGGCAGGA AGTCTGGTTA CGACTTGCAA AAGGAACAAT TCCAGCCAAG TTTCCCCCTG | 3720 |
| TTACTACATT TAGTGGCTAT TTATTATATC GTACATAAGT TAGTATGAAA AACAGACTAT | 3780 |
| CACCTTTATT GAGAAACAGC CAGTGTTTTC ATTTATCTTT GCTTGCACAT CTGCTCTGTT | 3840 |
| TTGGTTTTTC TACAGGAAAT GAAAATCAAC TTGTTTTTTT AATATGAGTA AACTTGTATG | 3900 |
| TCTATTTTAT AAAATTATTT GAATATTGTT TAATGTCTGA ATATGAAAGA GTTCTTGATC | 3960 |
| CTAAAGAAAT TTAGTGGCAC AGAAAACAAA GTGAATTTGT TAGCATAATT ATTCCTATTC | 4020 |
| TTATTTCTTC ATTTTAAGTC ATTGCAATGG AAAGTAATAT TATAAAACGG TAATTACAAC | 4080 |
| ATATTATCAG TCACAGTTTT CTTTCCAATT AAACACTTAA CTTTTGTTAT TCCCTGTATA | 4140 |
| TAAATATATA ACACACATTT TCTAGATTCA CAAATTTAAA TAAATTACTC AAAAAATG | |

Seq ID No: 101 Protein sequence:
Protein Accession #: NP_031377.1

| | |
|---|---|
| 1 11 21 31 41 51 | |
| \| \| \| \| \| \| | |
| MKGARLFVLL SSLWSGGIGL NNSKHSWTIP EDGNSQKTMP SASVPPNKIQ SLQILPTTRV | 60 |
| MSAEIATTPS ARTSEDSLLK STLPPSETSA PAEGVRNQTL TSTEKAEGVV KLQNLTLPTN | 120 |
| ASIKFNPGAE SVVLSNSTLK FLQSFARKSN EQATSLNTVG GTGGIGGVGG TGGVGNRAPR | 180 |
| ETYLSRGDSS SSQRTDYQKS NFETTRGKNW CAYVHTRLSP TVTLDNQVTY VPGGKGPCGW | 240 |
| TGGSCPQRSQ KISNPVYRMQ HKIVTSLDWR CCPGYSGPKC QLRAQEQQSL IHTNQAESHT | 300 |
| AVGRGVAEQQ QQQGCGDPEV MQKMTDQVNY QAMKLTLLQK KIDNISLTVN DVRNTYSSLE | 360 |
| GKVSEDKSRE FQSLLKGLKS KSINVLIRDT VREQFKIFQN DMQETVAQLF KTVSSLSEDL | 420 |
| ESTRQIIQKV NESVVSIAAQ QKFVLVQENR PTLTDIVELR NHIVNVRQEM TLTCEKPIKE | 480 |
| LEVKQTHLEG ALEQEHSRSI LYYESLNKTL SKLKEVHEQL LSTEQVSDQK NAPAAESVSN | 540 |
| NVTEYMSTLH ENIKKQSLMM LQMFEDLHIQ ESKINNLTVS LEMEKESLRG ECEDMLSKCR | 600 |
| NDFKFQLKDT EENLHVLNQT LAEVLFPMDN KMDKMSEQLN DLTYDMEILQ PLLEQGASLR | 660 |
| QTMTYEQPKE AIVIRKKIEN LTSAVNSLNP IIKELTKRHN LLRNEVQGRD DALERRINEY | 720 |
| ALEMEDGLNK TMTIINNAID FIQDNYALKE TLSTIKDNSE IHHKCTSDME TILTFIPQFH | 780 |
| RLNDSIQTLV NDNQRYNFVL QVAKTLAGIP RDEKLNQSNF QKMYQMFNET TSQVRKYQQN | 840 |
| MSHLEEKLLL TTKISKNEET RLQDIESKVT QTLTPYYISV KKGSVVTNER DQALQLQVLN | 900 |
| SRFKALEAKS IHLSINFFSL NKTLHEVLTM CHNASTSVSE LNATIPKWIK HSLPDIQLLQ | 960 |
| KGLTEFVEPI IQIKTQAALS NSTCCIDRSL PGSLANVVKS QKQVKSLPKK INALKKPTVN | 1020 |
| LTTVLIGRTQ RNTDNIIYPE EYSSCSRNPC QNGGTCINGR TSFTCACRHP FTGDNCTIKL | 1080 |
| VEENALAPDF SKGSYRYAPM VAFFASHTYG MTIPGPILFN NLDVNYGASY TPRTGKFRIP | 1140 |
| YLGVYVFKYT IESFSAHISG FLVVDGIDKL AFESENINSE IHCDRVLTGD ALLELNYCQE | 1200 |
| VWLRLAKGTI PAKFPPVTTF SGYLLYRT | |

Seq ID NO: 102 DNA sequence:
Nucleic Acid Accession #: NM_000873.2
Coding sequence: 57-884 (underlined sequences correspond to start and
stop codons)

| | |
|---|---|
| 1 11 21 31 41 51 | |
| \| \| \| \| \| \| | |
| ATCTCCCTCC AGGCAGCCCT TGGCTGGTCC CTGCGAGCCC GTGGAGACTG CCAGAGATGT | 60 |
| CCTCTTTCGG TTACAGGACC CTGACTGTGG CCCTCTTCAC CCTGATCTGC TGTCCAGGAT | 120 |
| CGGATGAGAA GGTATTCGAG GTACACGTGA GGCCAAAGAA GCTGGCGGTT GAGCCCAAAG | 180 |
| GGTCCCTCGA GGTCAACTGC AGCACCACCT GTAACCAGCC TGAAGTGGGT GGTCTGGAGA | 240 |
| CCTCTCTAAA TAAGATTCTG CTGGACGAAC AGGCTCAGTG GAAACATTAC TTGGTCTCAA | 300 |
| ACATCTCCCA TGACACGGTC CTCCAATGCC ACTTCACCTG CTCCGGGAAG CAGGAGTCAA | 360 |
| TGAATTCCAA CGTCAGCGTG TACCAGCCTC CAAGGCAGGT CATCCTGACA CTGCAACCCA | 420 |
| CTTTGGTGGC TGTGGGCAAG TCCTTCACCA TTGAGTGCAG GGTGCCCACC GTGGAGCCCC | 480 |
| TGGACAGCCT CACCCTCTTC CTGTTCCGTG GCAATGAGAC TCTGCACTAT GAGACCTTCG | 540 |
| GGAAGGCAGC CCCTGCTCCG CAGGAGGCCA CAGCCACATT CAACAGCACG GCTGACAGAG | 600 |
| AGGATGGCCA CCGCAACTTC TCCTGCCTGG CTGTGCTGGA CTTGATGTCT CGCGGTGGCA | 660 |
| ACATCTTTCA CAAACACTCA GCCCCGAAGA TGTTGGAGAT CTATGAGCCT GTGTCGGACA | 720 |
| GCCAGATGGT CATCATAGTC ACGGTGGTGT CGGTGTTGCT GTCCCTGTTC GTGACATCTG | 780 |
| TCCTGCTCTG CTTCATCTTC GGCCAGCACT TGCGCCAGCA GCGGATGGGC ACCTACGGGG | 840 |
| TGCGAGCGGC TTGGAGGAGG CTGCCCCAGG CCTTCCGGCC ATAGCAACCA TGAGTGGCAT | 900 |
| GGCCACCACC ACGGTGGTCA CTGGAACTCA GTGTGACTCC TCAGGGTTGA GGTCCAGCCC | 960 |
| TGGCTGAAGG ACTGTGACAG GCAGCAGAGA CTTGGGACAT TGCCTTTTCT AGCCCGAATA | 1020 |
| CAAACACCTG GACTT | |

Seq ID No: 103 Protein sequence:
Protein Accession #: NP_000864.1

| | |
|---|---|
| 1 11 21 31 41 51 | |
| \| \| \| \| \| \| | |
| MSSFGYRTLT VALFTLICCP GSDEKVFEV HVRPKKLAVEP KCSLEVNCST TCNQPEVGGL | 60 |
| ETSLNKILLD EQAQWKHYLV SNISHDTVL QCHFTCSGKQE SMNSNVSVYQ PPRQVILTLQ | 120 |
| PTLVAVGKSF TIECRVPTVE PLDSLTLFL FRGNETLHYET RGKAAPAPQE ATATFNSTAD | 180 |
| REDGHPNFSC LAVLDLMSRG GNIFNKHSAP KMLEIYEPVS DSQMVIIVTV VSVLLSLFVT | 240 |
| SVLLCFIFGQ HLRQQRMGTY GVRAAWRRLP QAFRP | |

Seq ID NO: 104 DNA sequence:
Nucleic Acid Accession #: NM_001795.2
Coding sequence: 121-2475 (underlined sequences correspond to start
and stop codons)

| | |
|---|---|
| 1 11 21 31 41 51 | |
| \| \| \| \| \| \| | |
| GACGGTCGGC TGACAGGCTC CACAGAGCTC CACTCACGCT CAGGCCCTGG ACGGACAGGC | 60 |
| AGTCCAACGG AACAGAAACA TCCCTCAGCC CCACAGGCAC GATCTGTTCC TCCTGGGAAG | 120 |
| ATGCAGAGGC TCATGATGCT CCTCGCCACA TCGGGCGCCT GCCTGGGCCT GCTGGCAGTG | 180 |
| GCAGCAGTGG CAGCAGCAGG TGCTAACCCT GCCCAACGGG ACACCCACAG CCTGCTGCCC | 240 |
| ACCCACCGGC GCCAAAAGAG AGATTGGATT TGGAACCAGA TGCACATTGA TGAAGAGAAA | 300 |
| AACACCTCAC TTCCCCATCA TGTAGGCAAG ATCAAGTCAA GCGTGAGTCG CAAGAATGCC | 360 |
| AAGTACCTGC TCAAAGGAGA ATATGTGGGC AAGGTCTTCC GGGTCGATGC AGAGACAGGA | 420 |
| GACGTGTTCG CCATTGAGAG GCTGGACCGG GAGAATATCT CAGAGTACCA CCTCACTGCT | 480 |
| GTCATTGTGG ACAAGGACAC TGGTGAAAAC CTGGAGACTC CTTCCAGCTT CACCATCAAA | 540 |
| GTTCATGACG TGAACGACAA CTGGCCTGTG TTCACGCATC GGTTGTTCAA TGCGTCCGTG | 600 |
| CCTGAGTCGT CGGCTGTGGG GACCTCAGTC ATCTCTGTGA CAGCAGTGGA TGCAGACGAC | 660 |
| CCCACTGTGG GAGACCACGC CTCTGTCATG TACCAAATCC TGAAGGGGAA AGAGTATTTT | 720 |
| GCCATCGATA ATTCTGGACG TATTATCACA ATAACGAAAA GCTTGGACCG AGAGAAGCAG | 780 |
| GCCAGGTATG AGATCGTGGT GGAAGCGCGA GATGCCCAGG GCCTCCGGGG GGACTCGGGC | 840 |
| ACGGCCACCG TGCTGGTCAC TCTGCAAGAC ATCAATGACA ACTTCCCCTT CTTCACCCAG | 900 |
| ACCAAGTACA CATTTGTCGT GCCTGAAGAC ACCCGTGTGG GCACCTCTGT GGGCTCTCTG | 960 |
| TTTGTTGAGG ACCCAGATGA GCCCCAGAAC CGGATGACCA AGTACAGCAT CTTGCGGGGC | 1020 |
| GACTACCAGG ACGCTTTCAC CATTGAGACA AACCCCGCCC ACAACGAGGG CATCATCAAG | 1080 |
| CCCATGAAGC CTCTGGATTA TGAATACATC CAGCAATACA GCTTCATCGT CGAGGCCACA | 1140 |
| GACCCCACCA TCGACCTCCG ATACATGAGC CCTCCCGCGG GAAACAGAGC CCAGGTCATT | 1200 |
| ATCAACATCA CAGATGTGGA CGAGCCCCCC ATTTTCCAGC AGCCTTTCTA CCACTTCCAG | 1260 |
| CTGAAGGAAA ACCAGAAGAA GCCTCTGATT GGCACAGTGC TGGCCATGGA CCCTGATGCG | 1320 |
| GCTAGGCATA GCATTGGATA CTCCATCCGC AGGACCACTG ACAAGGGCCA GTTCTTCCGA | 1380 |
| GTCACAAAAA AGGGGGACAT TTACAATGAG AAAGAACTGG ACAGAGAAGT CTACCCCTGG | 1440 |
| TATAACCTGA CTGTGGAGGC CAAAGAACTG GATTCCACTG GAACCCCCAC AGGAAAAGAA | 1500 |
| TCCATTGTGC AAGTCCACAT TGAAGTTTTG GATGAGAATG ACAATGCCCC GGAGTTTGCC | 1560 |
| AAGCCCTACC AGCCCAAAGT GTGTGAGAAC GCTGTCCATG GCCAGCTGGT CCTGCAGATC | 1620 |
| TCCGCAATAG ACAAGGACAT AACACCACGA AACGTGAAGT TCAAATTCAC CTTGAATACT | 1680 |
| GAGAACAACT TTACCCTCAC GGATAATCAC GATAACACGG CCAACATCAC AGTCAAGTAT | 1740 |
| GGGCAGTTTG ACCGGGAGCA TACCAAGGTC CACTTCCTAC CCGTGGTCAT CTCAGACAAT | 1800 |
| GGGATGCCAA GTCGCACGGG CACCAGCACG CTGACCGTGG CCGTGTGCAA GTGCAACGAG | 1860 |
| CAGGGCGAGT TCACCTTCTG CGAGGATATG GCCGCCCAGG TGGGCGTGAG CATCCAGGCA | 1920 |
| GTGGTAGCCA TCTTACTCTG CATCCTCACC ATCACAGTGA TCACCCTGCT CATCTTCCTG | 1980 |
| CGGCGGCGGC TCCGGAAGCA GGCCCGCGCG CACGGCAAGA GCGTGCCGGA GATCCACGAG | 2040 |
| CAGCTGGTCA CCTACGACGA GGAGGGCGGC GGCGAGATGG ACACCACCAG CTACGATGTG | 2100 |
| TCGGTGCTCA ACTCGGTGCG CCGCGGCGGG GCCAAGCCCC CGCGGCCCGC GCTGGACGCC | 2160 |
| CGGCCTTCCC TCTATGCGCA GGTGCAGAAG CCACCGAGGC ACGCGCCTGG GGCACACGGA | 2220 |
| GGGCCCGGGG AGATGGCAGC CATGATCGAG GTGAAGAAGG ACGAGGCGGA CCACGACGGC | 2280 |
| GACGGCCCCC CCTACGACAC GCTGCACATC TACGGCTACG AGGGCTCCGA GTCCATAGCC | 2340 |
| GAGTCCCTCA GCTCCCTGGG CACCGACTCA TCCGACTCTG ACGTGGATTA CGACTTCCTT | 2400 |
| AACGACTGGG GACCCAGGTT TAAGATGCTG GCTGAGCTGT ACGGCTCGGA CCCCCGGGAG | 2460 |
| GAGCTGCTGT ATTAGGCGGC CGAGGTCACT CTGGGCCTGG GGACCCAAAC CCCCTGCAGC | 2520 |
| CCAGGCCAGT CAGACGCCAG GCACCACAGC CTCCAAAAAT GGCAGTGACT CCCCAGCCCA | 2580 |
| GCACCCCTTC CTCGTGGGTC CCAGAGACCT CATCAGCCTT GGGATAGCAA ACTCCAGGTT | 2640 |
| CCTGAAATAT CCAGGAATAT ATGTCAGTGA TGACTATTCT CAAATGCTGG CAAATCCAGG | 2700 |
| CTGGTGTTCT GTCTGGGCTC AGACATCCAC ATAACCCTGT CACCCACAGA CCGCCGTCTA | 2760 |
| ACTCAAAGAC TTCCTCTGGC TCCCCAAGGC TGCAAAGCAA AACAGACTGT GTTTAACTGC | 2820 |
| TGCAGGGTCT TTTTCTAGGG TCCCTGAACG CCCTGGTAAG GCTGGTGAGG TCCTGGTGCC | 2880 |
| TATCTGCCTG GAGGCAAAGG CCTGGACAGC TTGACTTGTG GGGCAGGATT CTCTGCAGCC | 2940 |
| CATTCCCAAG GGAGACTGAC CATCATGCCC TCTCTCGGGA GCCCTAGCCC TGCTCCAACT | 3000 |
| CCATACTCCA CTCCAAGTGC CCCACCACTC CCCAACCCCT CTCCAGGCCT GTCAAGAGGG | 3060 |
| AGGAAGGGGC CCCATGGCAG CTCCTGACCT TGGGTCCTGA AGTGACCTCA CTGGCCTGCC | 3120 |
| ATGCCAGTAA CTGTGCTGTA CTGAGCACTG AACCACATTC AGGGAAATGG CTTATTAAAC | 3180 |
| TTTGAAGCAA CTGTGAATTC ATTCTGCAGG GGCAGTGGAG ATCAGGAGTG ACAGATCACA | 3240 |
| GGGTGAGGGC CACCTCCACA CCCACCCCCT CTGGAGAAGG CCTGGAAGAG CTGAGACCTT | 3300 |
| GCTTTGAGAC TCCTCAGCAC CCCTCCAGTT TTGCCTGAGA AGGGGCAGAT GTTCCCGGAG | 3360 |
| CAGAAGACGT CTCCCCTTCT CTGCCTCACC TGGTCGCCAA TCCATGCTCT CTTTCTTTTC | 3420 |
| TCTGTCTACT CCTTATCCCT TGGTTTAGAG GAACCCAAGA TGTGGCCTTT AGCAAAACTG | 3480 |
| GACAATGTCC AAACCCACTC ATGACTGCAT GACGGAGCCG AGCCATGTGT CTTTACACCT | 3540 |
| CGCTGTTGTC ACATCTCAGG GAACTGACCC TCAGGCACAC CTTGCAGAAG GCAAGGCCCT | 3600 |
| GCCCTGCCCA ACCTCTGTGG TCACCCATGC ATCTTCCACT GGAACGTTTC ACTGCAAACA | 3660 |
| CACCTTGGAG AAGTGGCATC AGTCAACAGA GAGGGGCAGC GAAGGAGACA CCAAGCTCAC | 3720 |
| CCTTCGTCAT GGACCGAGGT TCCCACTCTG GGCAAAGCCC CTCACACTGC AAGGGATTGT | 3780 |
| AGATAACACT GACTTGTTTG TTTTAACCAA TAACTAGCTT CTTATAATGA TTTTTTTACT | 3840 |
| AATGATACTT ACAAGTTTCT AGCTCTCACA GACATATAGA ATAAGGGTTT TTGCATAATA | 3900 |
| AGCAGGTTGT TATTTAGGTT AACAATATTA ATTCAGGTTT TTTAGTTGGA AAAACAATTC | 3960 |
| CTGTAACCTT CTATTTTCTA TAATTGTAGT AATTGCTCTA CAGATAATGT CTATATATTG | 4020 |
| GCCAAACTGG TGCATGACAA GTACTGTATT TTTTTATACC TAAATAAAGA AAAATCTTTA | 4080 |
| GCCTGGGCAA CAAAAAAA | |

Seq ID No: 105 Protein sequence:
Protein Accession #: NP_001786.1

| | |
|---|---|
| 1 11 21 31 41 51 | |
| \| \| \| \| \| \| | |
| MQRLMMLLAT SGACLGLLAV AAVAAAGANP AQRDTHSLLP THRRQKRDWI WNQMHIDEEK | 60 |
| NTSLPHHVGK IKSSVSRKNA KYLLKGEYVG KVPRVDAETG DVFAIERLDR ENISEYHLTA | 120 |
| VIVDKDTGEN LETPSSFTIK VHDVNDNWPV FTHRLFNASV PESSAVGTSV ISVTAVDADD | 180 |
| PTVGDHASVM YQILKGKEYF AIDNSGRIIT ITKSLDREKQ ARYEIVVEAR DAQCLRGDSG | 240 |
| TATVLVTLQD INDNFPFFTQ TKYTFVVPED TRVGTSVGSL FVEDPDEPQN RMTKYSILRG | 300 |
| DYQDAFTIET NPAHNEGIIK PMKPLDYEYI QQYSFIVEAT DPTIDLRYMS PPAGNRAQVI | 360 |
| INITDVDEPP IFQQPFYHFQ LKENQKKPLI GTVLAMDPDA ARESIGYSIR RTSDKGQFFR | 420 |
| VTKKGDIYNE KELDREVYPW YNLTVEAKEL DSTGTPTGKE SIVQVHIEVL DENDNAPEFA | 480 |
| KPYQPKVCEN AVHGQLVLQI SAIDKDITPR NVKFKFTLNT ENNFTLTDNN DNTANITVKY | 540 |
| GQFDREHTKV HFLPVVISDN GMPSRTGTST LTVAVCKCNE QGEFTFCEDM AAQVGVSIQA | 600 |
| VVAILLCILT ITVITLLIFL RRRLRKQARA HGKSVPEIHE QLVTYDEEGG GEMDTTSYDV | 660 |
| SVLNSVRRGG AKPPRPALDA RPSLYAQVQK PPRHAPGAHG GPGEMAAMIE VKKDEADHDG | 720 |
| DGPPYDTLNI YGYEGSESIA ESLSSLGTDS SDSDVDYDFL NDWGPRFKML AELYGSDPRE | 780 |
| ELLY | |

Seq ID NO: 106 DNA sequence:
Nucleic Acid Accession #: none found
Coding sequence: 1-474 (underlined sequences correspond to start and
stop codons)

| | |
|---|---|
| 1 11 21 31 41 51 | |
| \| \| \| \| \| \| | |
| ACAGTACTCT GTGCAAAAAA CCTGGTGAAA AAGGATTTTT TCCGACTTCC TGATCCATTT | 60 |
| GCTAAGGTGG TGGTTGATGG ATCTGGGCAA TGCCATTCTA CAGATACTGT GAAGAATACG | 120 |
| CTTGATCCAA AGTGGAATCA GCATTATGAC CTGTATATTG GAAAGTCTGA TTCAGTTACG | 180 |
| ATCAGTGTAT GGAATCACAA GAAGATCCAT AAGAAACAAG GTGCTGGATT TCTCGGTTGT | 240 |
| GTTCGTCTTC TTTCCAATGC CATCAACCGC CTCAAAGACA CTGGTTATCA GAGGTTGGAT | 300 |
| TTATGCAAAC TCGGGCCAAA TCACAATGAT ACAUTTAGAG GACAGATACT AGTAAGTCTT | 360 |
| CAGTCCAGAG ACCGAATAGG CACAGGAGGA CAAGTTGTGG ACTGCAGTCG TTTATTTGAT | 420 |
| AACGATTTAC CAGACGGAGC TCATTATTTG TGGACTTGGA AAGATAGATG TTAATGACTG | 480 |
| GAAGGTAAAC ACCCGGTTAA AACACTGTAC ACCAGACAGC AACATTGTCA AATGGTTCTG | 540 |
| GAAAGCTGTG GAGTTTTTTG ATGAAGAGCG ACGAGCAAGA TTGCTTCAGT TTGTGACAGG | 600 |
| ATCCTCTCGA GTGCCTCTGC AGGGCTTCAA AGCATTGCAA GGTGCTGCAG GCCCGAGACT | 660 |
| CTTTACCATA CACCAGATTG ATGCCTGCAC TAACAACCTG CCGAAAGCCC ACACTTGCTT | 720 |
| CAATCGAATA GACATTCCAC CCTATGAAAG CTATGAAAAG CTATATGAAA AGCTGCTAAC | 780 |
| AGCCATTGAA GAAACATGTG GATTTGCTGT GGAATGACAA GCTTCAAGGA TTTACCCAGG | 840 |
| AC | |

Seq ID No: 107 Protein sequence:
Protein Accession #: none found

| | |
|---|---|
| 1 11 21 31 41 51 | |
| \| \| \| \| \| \| | |
| TVLCAKNLVK KDFFRLPDPF AKVVVDGSGQ CNSTDTVKNT LDPKWNQHYD LYIGESGSYT | 60 |
| ISVNNHKKIH KKQGAGFLGC VRLLSNAINR LKDTGYQRLD LCKLGPNDND TVRGQIVVSL | 120 |
| QSRDRIGTGG QVVDCSRLFD NDLPDGAHYL WTWKDRC | |

Seq ID NO: 108 DNA sequence:
Nucleic Acid Accession #: NM_002318.1
Coding sequence: 248-2572 (underlined sequences correspond to start
and stop codons)

| | |
|---|---|
| 1 11 21 31 41 51 | |
| \| \| \| \| \| \| | |
| ACTCCAGCGC GCGGCTACCT ACGCTTGGTG CTTCCTTTCT CCAGCCATCG GAGACCAGAG | 60 |
| CCGCCCCCTC TGCTCGAGAA AGGGGCTCAG CGGCGGCGGA AGCGGAGGGG GACCACCGTG | 120 |
| GAGAGCGCGG TCCCAGCCCG GCCACTGCGG ATCCCTGAAA CCAAAAAGCT CCTGCTGCTT | 180 |
| CTGTACCCCG CCTGTCCCTC CCAGCTGCGC AGGGCCCCTT CGTGGGATCA TCAGCCCGAA | 240 |
| GACAGGGATG GAGAGGCCTC TGTGCTCCCA CCTCTGCAGC TGCCTGGCTA TGCTGGCCCT | 300 |
| CCTGTCCCCC CTGAGCCTGG CACAGTATGA CAGCTGGCCC CATTACCCCG AGTACTTCCA | 360 |
| GCAACCGGCT CCTGAGTATC ACCAGCCCCA GGCCCCCGCC AACGTGGCCA AGATTCAGCT | 420 |
| GCGCCTGGCT GGGCAGAAGA GGAAGCACAG CGAGGGCCGG GTGGAGGTGT ACTATGATGG | 480 |
| CCAGTGGGGC ACCGTGTGCG ATGACGACTT CTCCATCCAC GCTGCCCACG TCGTCTGCCG | 540 |
| GGAGCTGGGC TATGTGGAGG CCAAGTCCTG GACTGCCAGC TCCTCCTACG GCAAGGGAGA | 600 |
| AGGGCCCATC TGGTTAGACA ATCTCCACTG TACTGGCAAC GAGGCGACCC TTGCAGCATG | 660 |
| CACCTCCAAT GGCTGGGGCG TCACTGACTG CAAGCACACG GAGGATGTCG GTGTGGTGTG | 720 |
| CAGCGACAAA AGGATTCCTG GGTTCAAATT TGACAATTCG TTGATCAACC AGATAGAGAA | 780 |
| CCTGAATATC CAGGTGGAGG ACATTCGGAT TCGAGCCATC CTCTCAACCT ACCGCAAGCG | 840 |
| CACCCCAGTG ATGGAGGGCT ACGTGGAGGT GAAGGAGGGC AAGACCTGGA AGCAGATCTG | 900 |
| TGACAAGCAC TGGACGGCCA AGAATTCCCG CGTGGTCTGC GGCATGTTTG GCTTCCCTGG | 960 |
| GGAGAGGACA TACAATACCA AAGTGTACAA AATGTTTGCC TCACGGAGGA AGCAGCGCTA | 1020 |
| CTGGCCATTC TCCATGGACT GCACCGGCAC AGAGGCCCAC ATCTCCAGCT GCAAGCTGGG | 1080 |
| CCCCCAGGTG TCACTGGACC CCATGAAGAA TGTCACCTGC GAGAATGGGC TGCCGGCCGT | 1140 |
| GGTGAGTTGT GTGCCTGGGC AGGTCTTCAG CCCTGACGGA CCCTCGAGAT TCCGGAAAGC | 1200 |
| ATACAAGCCA GAGCAACCCC TGGTGCGACT GAGAGGCGGT GCCTACATCG GGGAGGGCCG | 1260 |
| CGTGGAGGTG CTCAAAAATG GAGAATGGGG GACCGTCTGC GACGACAAGT GGGACCTGGT | 1320 |
| GTCGGCCAGT GTGGTCTGCA GAGAGCTGGG CTTTGGGAGT GCCAAAGAGG CAGTCACTGG | 1380 |
| CTCCCGACTG GGGCAAGGGA TCGGACCCAT CCACCTCAAC GAGATCCAGT GCACAGGCAA | 1440 |
| TGAGAAGTCC ATTATAGACT GCAAGTTCAA TGCCGAGTCT CAGGGCTGCA ACCACGAGGA | 1500 |
| GGATGCTGGT GTGAGATGCA ACACCCCTGC CATGGGCTTG CAGAAGAAGC TGCGCCTGAA | 1560 |
| CGGCGGCCGC AATCCCTACG AGGGCCGAGT GGAGGTGCTG GTGGAGAGAA ACGGGTCCCT | 1620 |
| TGTGTGGGGG ATGGTGTGTG GCCAAAACTG GGGCATCGTG GAGGCCATGG TGGTCTGCCG | 1680 |
| CCAGCTGGGC CTGGGATTCG CCAGCAACGC CTTCCAGGAG ACCTGGTATT GGCACGGAGA | 1740 |
| TGTCAACAGC AACAAAGTGG TCATGAGTGG AGTGAAGTGC TCGGGAACGG AGCTGTCCCT | 1800 |
| GGCGCACTGC CGCCACGACG GGGAGGACGT GGCCTGCCCC CAGGGCGGAG TGCAGTACGG | 1860 |
| GGCCGGAGTT GCCTGCTCAG AAACCGCCCC TGACCTGGTC CTCAATGCGG AGATGGTGCA | 1920 |
| GCAGACCACC TACCTGGAGG ACCGGCCCAT GTTCATGCTG CAGTGTGCCA TGGAGGAGAA | 1980 |
| CTGCCTCTCG GCCTCAGCCG CGCAGACCGA CCCCACCACG GGCTACCGCC GGCTCCTGCG | 2040 |
| CTTCTCCTCC CAGATCCACA ACAATGGCCA GTCCGACTTC CGGCCCAAGA ACGGCCGCCA | 2100 |
| CGCGTGGATC TGGCACGACT GTCACAGGCA CTACCACAGC ATGGAGGTGT TCACCCACTA | 2160 |
| TGACCTGCTG AACCTCAATG GCACCAAGGT GGCAGAGGGC CACAAGGCCA GCTTCTGCTT | 2220 |
| GGAGGACACA GAATGTGAAG GAGACATCCA GAAGAATTAC GAGTGTGCCA ACTTCGGCGA | 2280 |
| TCAGGGCATC ACCATGGGCT GCTGGGACAT GTACCGCCAT GACATCGACT GCCAGTGGGT | 2340 |
| TGACATCACT GACGTGCCCC CTGGAGACTA CCTGTTCCAG GTTGTTATTA ACCCCAACTT | 2400 |
| CGAGGTTGCA GAATCCGATT ACTCCAACAA CATCATGAAA TGCAGGAGCC GCTATGACGG | 2460 |
| CCACCGCATC TGGATGTACA ACTGCCACAT AGGTGGTTCC TTCAGCGAAG AGACGGAAAA | 2520 |
| AAAGTTTGAG CACTTCAGCG GGCTCTTAAA CAACCAGCTG TCCCCGCAGT AAAGAAGCCT | 2580 |
| GCGTGGTCAA CTCCTGTCTT CAGGCCACAC CACATCTTCC ATGGGACTTC CCCCCAACAA | 2640 |
| CTGAGTCTGA ACGAATGCCA CGTGCCCTCA CCCAGCCCGG CCCCCACCCT GTCCAGACCC | 2700 |
| CTACAGCTGT GTCTAAGCTC AGGAGGAAAG GGACCCTCCC ATCATTCATG GGGGGCTGCT | 2760 |
| ACCTGACCCT TGGGGCCTGA GAAGGCCTTG GGGGGGTGGG GTTTGTCCAC AGAGCTGCTG | 2820 |
| GAGCAGCACC AAGAGCCAGT CTTGACCGGG ATGAGGCCCA CAGACAGGTT GTCATCAGCT | 2880 |
| TGTCCCATTC AAGCCACCGA GCTCACCACA GACACAGTGG AGCCGCGCTC TTCTCCAGTG | 2940 |
| ACACGTGGAC AAATGCGGGC TCATCAGCCC CCCCAGAGAG GGTCAGGCCG AACCCCATTT | 3000 |
| CTCCTCCTCT TAGGTCATTT TCAGCAAACT TGAATATCTA GACCTCTCTT CCAATGAAAC | 3060 |
| CCTCCAGTCT ATTATAGTCA CATAGATAAT GGTGCCACGT GTTTTCTGAT TTGGTGAGCT | 3120 |
| CAGACTTGGT GCTTCCCTCT CCACAACCCC CACCCCTTGT TTTTCAAGAT ACTATTATTA | 3180 |
| TATTTTCACA GACTTTTGAA GCACAAATTT ATTGGCATTT AATATTGGAC ATCTGGGCCC | 3240 |
| TTGGAAGTAC AAATCTAAGG AAAAACCAAC CCACTGTGTA AGTGACTCAT CTTCCTGTTG | 3300 |
| TTCCAATTCT GTGGGTTTTT GATTCAACGG TGCTATAACC AGGGTCCTGG GTGACAGGGC | 3360 |
| GCTCACTGAG CACCATGTGT CATCACAGAC ACTTACACAT ACTTGAAACT TGGAATAAAA | 3420 |
| GAAAGATTTA TG | |

Seq ID No: 109 Protein sequence:
Protein Accession #: NP_002309.1

| | |
|---|---|
| 1 11 21 31 41 51 | |
| \| \| \| \| \| \| | |
| MERPLCSHLC SCLAMLALLS PLSLAQYDSW PHYPEYFQQP APEYHQPQAP ANVAKIQLRL | 60 |
| AGQKRKNSEG RVEVYYDGQW GTVCDDDFSI HAAHVVCREL GYVEAKSWTA SSSYGKGEGP | 120 |
| IWLDNLHCTG NEATLAACTS NGWGVTDCKH TEDVGVVCSD KRIPGFKFDN SLINQIENLN | 180 |
| IQVEDIRIRA ILSTYRKRTP VMEGYVEVKE GKTWRQICDK HWTAKNSRVV CGMFGFPGER | 240 |
| TYNTKVYKMF ASRRKQRYWP FSMDCTGTEA HISSCKLGPQ VSLDPNKNVT CENGLPAVVS | 300 |
| CVPGQVFSPD GPSRFRKAYK PEQPLVRLRG GAYIGEGRVE VLKNGEWGTV CDDKWDLVSA | 360 |
| SVVCRELGFG SAKEAVTGSR LGQGIGPIHL NEIQCTGNEK SIIDCKFNAE SQGCNHEEDA | 420 |
| GVRCNTPAMG LQKKLRLNGG RNPYEGRVEV LVERNGSLVW GMVCGQNWGI VEANVVCRQL | 480 |
| GLGFASNAFQ ETWYWHGDVN SNKVVMSGVK CSGTELSLAN CRHDGEDVAC PQGGVQYGAG | 540 |
| VACSETAPDL VLNAEMVQQT TYLEDRPMFM LQCAMEENCL SASAAQTDPT TGYRRLLRFS | 600 |
| SQIHNNGQSD FRPKNGRHAW IWHDCHRHYH SMEVFTHYDL LNLNGTKVAE GHKASFCLED | 660 |
| TECEGDIQKN YECANFGDQG ITMGCWDMYR HDIDCQWVDI TDVPPGDYLF QVVINPNFEV | 720 |
| AESDYSNNIM KCRSRYDCHR IWMYNCHICG SFSEETEKKF EHFSGLLNNQ LSPQ | |

Seq ID NO: 110 DNA sequence:
Nucleic Acid Accession #: none found, CAT_73007_3
Coding sequence: 1-495 (underlined sequences correspond to start and
stop codons)

| | |
|---|---|
| 1 11 21 31 41 51 | |
| \| \| \| \| \| \| | |
| CGGACGCGTG GGTCGACCCA CGCGTCCGCC CACGCGTCCG TATGGACAGA GCCTCCACTG | 60 |
| GCTGCTGCCT GCCCGCCACA TACCCAGCTG ACATGGGCAC CGCAGGAGCC ATGCAGCTGT | 120 |
| CTGGGTGATC CTGGGCTTCC TCCTGTTCCG AGGCCACAAC TCCCAGCCCA CAATGACCCA | 180 |
| ACCTCTAGCT CTCAGGGAGG CCTTGGCCGT CTAAGTCTGA CCACAGAGCC AGTTTCTTCC | 240 |
| ACCCAGGATA CATCCCTTCC TCAGAGGCTA ACAGGCCAAG CCATCTGTCC AGCACTGGTA | 300 |
| CCCAGGCGCA GGTGTCCCCA GCAGTGGAAG AGACGGAGGC ACAAGCAGAG ACACATTTCA | 360 |
| ACTGTTCCCC CCAATTCAAC CACCATGAGC CTGAGCATGA GGGAAGATGC GACCATCCTG | 420 |
| CCAGCCCCAC GTCAGAGACT GTGCTCACTG TGGCTGCATT TGGGATGGAG TCGGGTGGAG | 480 |
| GCCCACTCTG GCTAGGGGGC GGCAGGCTGA GAGCTCACCT GTTCAGCAGA GAAGTGGAAC | 540 |
| CACTTTGCTC CTGGAGCCTG TCTACCACAG TGTTATCAGC TTCATTGTCA TCCTGGTGGT | 600 |
| GTGGTGATCA TCCTAGTTGG TGTGGTCAGC CTGAGGGTTC AGTGTCGGAA GAGCAAGGAG | 660 |
| TCTGAAGATC CCAGAACCTG GGAGTACAGG GCGTGTCTGA CAAGCTGGTC ACAGACCATG | 720 |
| GCGAGAACGA CAGCATCGCC CATTATCACA TGGAAGACAT CACACGACTT AGGGCAACAC | 780 |
| GCACTCAGCA GCGAGCATCA AACGAGCCTA CGCATGGCCC AGACTGAGAG CAAGCACAAA | 840 |
| GGGC | |

Seq ID No: 111 Protein sequence:
Protein Accession #: none found, CAT_73007_3

| | |
|---|---|
| 1 11 21 31 41 51 | |
| \| \| \| \| \| \| | |
| RTRGSTHASA HASVWTEPPL AAACPPHTQL TWAPQEPCSC LGDPGLPPVP RPQLPAHNDP | 60 |
| TSSSQGGLGG LSLTTEPVSS TQDTSLPQRL TGQAICPALV PRRRCPQQWK RRRHKQRHIS | 120 |
| TVPPNSTTMS LSMREDATIL PAPRQRLCSL WLHLGWSRVE AHSG | |

Seq ID NO: 112 DNA sequence:
Nucleic Acid Accession #: NM_005424.1
Coding sequence: 37-3453 (underlined sequences correspond to start and
stop codons)

| | |
|---|---|
| 1 11 21 31 41 51 | |
| \| \| \| \| \| \| | |
| CGCTCGTCCT GGCTGGCCTG GGTCGGCCTC TGGAGTATGG TCTGGCGGGT GCCCCCTTTC | 60 |
| TTGCTCCCCA TCCTCTTCTT GGCTTCTCAT GTGGGCGCGG CGGTGGACCT GACGCTGCTG | 120 |
| GCCAACCTGC GGCTCACGGA CCCCCAGCGC TTCTTCCTGA CTTGCGTGTC TGGGGAGGCC | 180 |
| GGGGCGGGGA GGGGCTCGGA CGCCTGGGGC CCGCCCCTGC TGCTGGAGAA GGACGACCGT | 240 |
| ATCGTGCGCA CCCCGCCCGG GCCACCCCTG CGCCTGGCGC GCAACGGTTC GCACCAGGTC | 300 |
| ACGCTTCGCG GCTTCTCCAA GCCCTCGGAC CTCGTGGGCG TCTTCTCCTG CGTGGGCGGT | 360 |
| GCTGGGGCGC GGCCCACGCG CGTCATCTAC GTGCACAACA GCCCTGGAGC CCACCTGCTT | 420 |
| CCAGACAAGG TCACACACAC TGTGAACAAA GGTGACACCG CTGTACTTTC TGCACGTGTG | 480 |
| CACAAGGAGA AGCAGACAGA CGTGATCTGC AAGAGCAACG GATCCTACTT CTACACCCTG | 540 |
| GACTGGCATG AAGCCCAGGA TGGGCGGTTC CTGCTGCAGC TCCCAAATGT GCAGCCACCA | 600 |
| TCGAGCGGCA TCTACAGTGC CACTTACCTG GAAGCCAGCC CCCTGGGCAG CGCCTTCTTT | 660 |
| CGGCTCATCG TGCGGGGTTG TGCCGCTGGG CGCTGGGGGC CAGGCTGTAC CAAGGAGTGC | 720 |
| CCAGGTTGCC TACATGGAGG TGTCTGCCAC GACCATGACG GCGAATGTGT ATGCCCCCCT | 780 |
| GGCTTCACTG GCACCCGCTG TGAACAGGCC TGCAGAGAGG GCCGTTTTGG GCAGAGCTGC | 840 |
| CAGGAGCAGT GCCCAGGCAT ATCAGGCTGC CGGGGCCTCA CCTTCTGCCT CCCAGACCCC | 900 |
| TATGGCTGCT CTTGTGGATC TGGCTGGAGA GGAAGCCAGT GCCAAGAAGC TTGTGCCCCT | 960 |
| GGTCATTTTG GGGCTGATTG CCGACTCCAG TGCCAGTGTC AGAATGGTGG CACTTGTGAC | 1020 |
| CGGTTCAGTG GTTGTGTCTG CCCCTCTGGG TGGCATGGAG TGCACTGTGA GAAGTCAGAC | 1080 |
| CGGATCCCCC AGATCCTCAA CATGGCCTCA GAACTGGAGT TCAACTTAGA GACGATGCCC | 1140 |
| CGGATCAACT GTGCAGCTGC AGGGAACCCC TTCCCCGTGC GGGGCAGCAT AGAGCTACGC | 1200 |
| AAGCCAGACG GCACTGTGCT CCTGTCCACC AAGGCCATTG TGGAGCCAGA GAAGACCACA | 1260 |
| GCTGAGTTCG AGGTGCCCCG CTTGGTTCTT GCGGACAGTG GGTTCTGGGA GTGCCGTGTG | 1320 |
| TCCACATCTG GCGGCCAAGA CAGCCGGCGC TTCAAGGTCA ATGTGAAAGT GCCCCCCGTG | 1380 |
| CCCCTGGCTG CACCTCGGCT CCTGACCAAG CAGAGCCGCC AGCTTGTGGT CTCCCCGCTG | 1440 |
| GTCTCGTTCT CTGGGGATGG ACCCATCTCC ACTGTCCGCC TGCACTACCG GCCCCAGGAC | 1500 |
| AGTACCATGG ACTGGTCGAC CATTGTGGTG GACCCCAGTG AGAACGTGAC GTTAATGAAC | 1560 |
| CTGAGGCCAA AGACAGGATA CAGTGTTCGT GTGCAGCTGA GCCGGCCAGG GGAAGGAGGA | 1620 |
| GAGGGGGCCT GGGGGCCTCC CACCCTCATG ACCACAGACT GTCCTGAGCC TTTGTTGCAG | 1680 |
| CCGTGGTTGG AGGGCTGGCA TGTGGAAGGC ACTGACCGGC TGCGAGTGAG CTGGTCCTTG | 1740 |
| CCCTTGGTGC CCGGGCCACT GGTGGGCGAC GGTTTCCTGC TGCGCCTGTG GGACGGGACA | 1800 |
| CGGGGGCAGG AGCGGCGGGA GAACGTCTCA TCCCCCCAGG CCCGCACTGC CCTCCTGACG | 1860 |
| GGACTCACGC CTGGCACCCA CTACCAGCTG GATGTGCAGC TCTACCACTG CACCCTCCTG | 1920 |
| GGCCCGGCCT CGCCCCCTGC ACACGTGCTT CTGCCCCCCA GTGGGCCTCC AGCCCCCCGA | 1980 |
| CACCTCCACG CCCAGGCCCT CTCAGACTCC GAGATCCAGC TGACATGGAA GCACCCGGAG | 2040 |
| GCTCTGCCTG GGCCAATATC CAAGTACGTT GTGGAGGTGC AGGTGGCTCG GGGTGCAGGA | 2100 |
| GACCCACTGT GGATAGACGT GGACAGGCCT GAGGAGACAA GCACCATCAT CCGTGGCCTC | 2160 |
| AACGCCAGCA CGCGCTACCT CTTCCGCATG CGGGCCAGCA TTCAGGGGCT CGGGGACTGG | 2220 |
| AGCAACACAG TAGAAGAGTC CACCCTGGGC AACGGGCTGC AGGCTGAGGG CCCAGTCCAA | 2280 |
| GAGAGCCGGG CAGCTGAAGA GGGCCTGGAT CAGCAGCTGA TCCTGGCGGT GGTGGGCTCC | 2340 |
| GTGTCTGCCA CCTGCCTCAC CATCCTGGCC GCCCTTTTAA CCCTGGTGTG CATCCGCAGA | 2400 |
| AGCTGCCTGC ATCGGAGACG CACCTTCACC TACCAGTCAG GCTCGGGCGA CGAGACCATC | 2460 |
| CTGCAGTTCA GCTCAGGGAC CTTGACACTT ACCCGGCGGC CAAAACTGCA GCCCGAGCCC | 2520 |
| CTGAGCTACC CAGTGCTAGA GTGGGAGGAC ATCACCTTTG AGGACCTCAT CGGGGAGGGG | 2580 |
| AACTTCGGCC AGGTCATCCG GGCCATGATC AAGAAGGACG GGCTGAAGAT GAACGCAGCC | 2640 |
| ATCAAAATGC TGAAAGAGTA TGCCTCTGAA AATGACCATC GTGACTTTGC GGGAGAACTG | 2700 |
| GAAGTTCTGT GCAAATTGGG GCATCACCCC AACATCATCA ACCTCCTGGG GGCCTGTAAG | 2760 |
| AACCGAGGTT ACTTGTATAT CGCTATTGAA TATGCCCCCT ACGGGAACCT GCTAGATTTT | 2820 |
| CTGCGGAAAA GCCGCGTCCT AGAGACTGAC CCAGCTTTTG CTCGAGAGCA TGGGACAGCC | 2880 |
| TCTACCCTTA GCTCCCGGCA GCTGCTGCGT TTCGCCAGTG ATGCGGCCAA TGGCATGCAG | 2940 |
| TACCTGAGTG AGAAGCAGTT CATCCACASG GACCTGGCTG CCCGGAATGT GCTGGTCGGA | 3000 |
| GAGAACCTAG CCTCCAAGAT TGCAGACTTC GGCCTTTCTC GGGGAGAGGA GGTTTATGTG | 3060 |
| AAGAAGACGA TGGGGCGTCT CCCTGTGCGC TGGATGGCCA TTGAGTCCCT GAACTACAGT | 3120 |
| GTCTATACCA CCAAGAGTGA TGTCTGGTCC TTTGGAGTCC TTCTTTGGGA GATAGTGAGC | 3180 |
| CTTGGAGGTA CACCCTACTG TGGCATGACC TGTGCCGAGC TCTATGAAAA GCTGCCCCAG | 3240 |
| GGCTACCGCA TGGAGCAGCC TCGAAACTGT GACGATGAAG TGTACGAGCT GATGCGTCAG | 3300 |
| TGCTGGCGGG ACCGTCCCTA TGAGCGACCC CCCTTTGCCC AGATTGCGCT ACAGCTAGGC | 3360 |
| CGCATGCTGG AAGCCAGGAA GGCCTATGTG AACATGTCGC TGTTTGAGAA CTTCACTTAC | 3420 |
| GCGGGCATTG ATGCCACAGC TGAGGAGGCC TGAGCTGCCA TCCAGCCAGA ACGTGGCTCT | 3480 |
| GCTGGCCGGA GCAAACTCTG CTGTCTAACC TGTGACCAGT CTGACCCTTA CAGCCTCTGA | 3540 |
| CTTAAGCTGC CTCAAGGAAT TTTTTTAACT TAAGGGAGAA AAAAAGGGAT CTGGGGATGG | 3600 |
| GGTGGGCTTA GGGGAACTGG GTTCCCATGC TTTGTAGGTG TCTCATAGCT ATCCTGGGCA | 3660 |
| TCCTTCTTTC TAGTTCAGCT GCCCCACAGG TGTGTTTCCC ATCCCACTGC TCCCCCAACA | 3720 |
| CAAACCCCCA CTCCAGCTCC TTCGCTTAAG CCAGCACTCA CACCACTAAC ATGCCCTGTT | 3780 |
| CAGCTACTCC CACTCCCGGC CTGTCATTCA GAAAAAAATA AATGTTCTAA TAAGCTCCAA | 3840 |

Seq ID No: 113 Protein sequence:
Protein Accession #: NP_005415.1

| | |
|---|---|
| 1 11 21 31 41 51 | |
| \| \| \| \| \| \| | |
| MVWRVPPFLL PILFLASHVC AAVDLTLLAN LRLTDPQRFF LTCVSGEAGA GRGSDAWGPP | 60 |
| LLLEKDDRIV RTPPGPPLRL ARNGSHQVTL RGFSKPSDLV GVFSCVCCAG ARRTRVIYVH | 120 |
| NSPGAHLLPD KVTHTVNKGD TAVLSARVHK EKQTDVIWKS NGSYFYTLDW NEAQDGRYLL | 180 |
| QLPNVQPPSS GIYSATYLEA SPLGSAFFRL IVRGCGAGRW GPGCTKECPG CLHCCVCHDH | 240 |
| DCECVCPPGF TGTRCEQACR EGRFGQSCQE QCPGISGCRG LTFCLPDPYG CSCGSGWRGS | 300 |
| QCQEACAPGH FGADCRLQCQ CQNGGTCDRF SGCVCPSGWH GVHCEKSDRI PQILNMASEL | 360 |
| EFNLETMPRI NCAAAGNPFP VRGSIELRKP DGTVLLSTKA IVEPEKTTAE FEVPRLVLAD | 420 |
| SGFWECRVST SGGQDSRRFK VNVKVPPVPL AAPRLLTKQS RQLVVSPLVS FSGDGPISTV | 480 |
| RLHYRPQDST MDWSTIVVDP SENVTLMNLR PKTGYSVRVQ LSRPGEGGEG AWCPPTLMTT | 540 |
| DCPEPLLQPW LEGWHVEGTD RLRVSWSLPL VPCPLVCDGF LLRLWDGTRG QERRENVSSP | 600 |
| QARTALLTGL TPGTHYQLDV QLYHCTLLGP ASPPAHVLLP PSGPPAPRHL HAQALSDSEI | 660 |
| QLTWKHPEAL PGPISKYVVE VQVAGGAGDP LWIDVDRPEE TSTIIRGLNA STRYLFRMRA | 720 |
| SIQGLGDWSN TVEESTLGNG LQAEGPVQES RAAEEGLDQQ LILAVVGSVS ATCLTILAAL | 780 |
| LTLVCIRRSC LHRRRTFTYQ SGSGNETILQ FSSGTLTLTR RPKLQPEPLS YPVLEWEDIT | 840 |
| FEDLIGEGNF GQVIRANIKK DGLKNNAAIK MLKEYASEND HRDFACELEV LCKLGHHPNI | 900 |
| INLLGACKNR GYLYIAIEYA PYGNLLDFLR KSRVLETDPA FAREHGTAST LSSRQLLRFA | 960 |
| SDAANGMQYL SEKQFIHRDL AARNVLVGEN LASKIADFGL SRGEEVYVKK TMGRLPVRWM | 1020 |
| AIESLNYSVY TTKSDVWSFG VLLWEIVSLG GTPYCGMTCA ELYEKLPQGY RMEQPRNCDD | 1080 |
| EVYELMRQCW RDRPYERPPF AQIALQLGRM LEARKAYVNM SLFENFTYAG IDATAEEA | |

Seq ID NO: 114 DNA sequence:
Nucleic Acid Accession #: NM_002632.1
Coding sequence: 322-771 (underlined sequences correspond to start and stop codons)

| | |
|---|---|
| 1 11 21 31 41 51 | |
| \| \| \| \| \| \| | |
| GGGATTCGGG CCGCCCAGCT ACGGGAGGAC CTGGAGTGGC ACTGGGCGCC CGACGGACCA | 60 |
| TCCCCGGGAC CCGCCTGCCC CTCGGCGCCC CGCCCCGCCG GGCCGCTCCC CGTCGGGTTC | 120 |
| CCCAGCCACA GCCTTACCTA CGGGCTCCTG ACTCCGCAAG GCTTCCAGAA GATGCTCGAA | 180 |
| CCACCGGCCG GGGCCTCGGG GCAGCAGTGA GGGAGGCGTC CAGCCCCCCA CTCAGCTCTT | 240 |
| CTCCTCCTGT GCCAGGGGCT CCCCGGGGGA TGAGCATGGT GGTTTTCCCT CGGAGCCCCC | 300 |
| TGGCTGGGGA CGTCTGAGAA GATGCCGGTC ATGACGCTGT TCCCTTGCTT CCTGCAGCTC | 360 |
| CTGGCCGGGC TGGCGCTGCC TGCTGTGCCC CCCCAGCAGT GGGCCTTGTC TGCTGGGAAC | 420 |
| GGCTCGTCAG AGGTGGAAGT GGTACCCTTC CAGGAAGTGT GGGGCCGCAG CTACTGCCGG | 480 |
| GCGCTGGAGA GGCTGGTGGA CGTCGTGTCC GAGTACCCCA GCGAGGTGGA GCACATGTTC | 540 |
| AGCCCATCCT GTGTCTCCCT GCTGCGCTGC ACCGGCTGCT GCCGCGATGA GAATCTGCAC | 600 |
| TGTGTGCCGG TGGAGACGGC CAATGTCACC ATGCAGCTCC TAAAGATCCG TTCTGGGGAC | 660 |
| CGGCCCTCCT ACGTGCAGCT GACGTTCTCT CAGCACGTTC GCTGCGAATG CCGGCCTCTG | 720 |
| CGGGAGAAGA TGAAGCCGGA AAGGTGCGGC GATGCTGTTC CCCGGAGGTA ACCCACCCCT | 780 |
| TGGAGGAGAG AGACCCCGCA CCCGGCTCCT GTATTTATTA CCGTCACACT CTTCAGTGAC | 840 |
| TCCTGCTGGT ACCTGCCCTC TATTTATTAG CCAACTGTTT CCCTGCTGAA TGCCTCGCTC | 900 |
| CCTTCAAGAC GAGGGGCAGG GAAGGACAGG ACCCTCAGGA ATTCAGTGCC TTCAACAACG | 960 |
| TGAGAGAAAG AGAGAAGCCA GCCACAGACC CCTGGGAGCT TCCGCTTTGA AAGAAGCAAG | 1020 |
| ACACGTGGCC TCGTGAGGGG CAAGCTAGGC CCCAGAGGCC CTGGAGGTCT CCAGGGGCCT | 1080 |
| GCAGAAGGAA AGAAGGGGGC CCTGCTACCT GTTCTTGGGC CTCAGGCTCT GCACAGACAA | 1140 |
| GCAGCCCTTG CTTTCGGAGC TCCTGTCCAA AGTAGGGATG CGGATTCTGC TGGGGCCGCC | 1200 |
| ACGGCCTGGT GGTGGGAAGG CCGGCAGCGG GCGGAGGGGA TTCAGCCACT TCCCCCTCTT | 1260 |
| CTTCTGAAGA TCAGAACATT CAGCTCTGGA GAACAGTGGT TGCCTGGGCG CTTTTGCCAC | 1320 |
| TCCTTGTCCC CCGTGATCTC CCCTCACACT TTGCCATTTG CTTGTACTGG GACATTGTTC | 1380 |
| TTTCCGGCCG AGCTGCCACC ACCCTGCCCC CACTAAGAGA CACATACAGA GTGGGCCCCG | 1440 |
| GGCTGGAGAA AGAGCTGCCT GGATGAGAAA CAGCTCAGCC AGTGGGGATG AGGTCACCAG | 1500 |
| GGGAGGAGCC TGTGCGTCCC AGCTGAAGGC AGTGGCAGGG GAGCAGGTTC CCCAAGGGCC | 1560 |
| CTGGCACCCC CACAAGCTGT CCCTGCAGGG CCATCTGACT GCCAAGCCAG ATTCTCTTGA | 1620 |
| ATAAAGTATT CTAGTGTGGA AACGC | |

Seq ID No: 115 Protein sequence:
Protein Accession #: NP_002623.1

| | |
|---|---|
| 1 11 21 31 41 51 | |
| \| \| \| \| \| \| | |
| MPVMRLFPCF LQLLACLALP AVPPQQWALS AGNGSSEVEV VPFQEVWGRS YCRALERLVD | 60 |
| VVSEYPSEVE HMFSPSCVSL LRCTGCCGDE NLHCVPVETA NVTMQLLKIR SGDRPSYVEL | 120 |
| TFSQHVRCEC RPLREKNKPE RCGDAVPRR | |

Seq ID NO: 116 DNA sequence:
Nucleic Acid Accession #: NM_007361.1
Coding sequence: 1-4131 (underlined sequences correspond to start and
stop codone)

| | |
|---|---|
| 1 11 21 31 41 51 | |
| \| \| \| \| \| \| | |
| ATGGAGGGGG ACCGGGTGGC CGGGCGGCCG GTGCTGTCGT CGTTACCAGT GCTACTGCTG | 60 |
| CTGCAGTTGC TAATGTTGCG GGCCGCGGCG CTGCACCCAG ACGAGCTCTT CCCACACGGG | 120 |
| GAGTCGTGGT GGGACCAGCT CCTGCAGGAA GGCGACGACG TAAAGCTCAG CCGTGGTGAA | 180 |
| GCTGGCGAAT CCCCTGCACT TCTTACGAAG CCCGATTCAG CAACCTCTAC GTGGGCACCA | 240 |
| ACGGCATCAT CTCCACTCAG GACTTCCCCA GGGAAACGCA GTATGTGGAC TATGATTTCC | 300 |
| CCACCGACTT CCCGGCCATC GCCCCTTTTC TGGCGGACAT CGACACGAGC CACGGCAGAG | 360 |
| GCCGAGTCCT GTACCGAGAG GACACCTCCC CCGCAGTGCT GGGCCTGGCC GCCCGCTATG | 420 |
| TGCGCGCTGG CTTCCCGCGC TCTGCGCGCT TTTTACCCCC ACCCACGCCT TCCTGGCCAC | 480 |
| CTGGGAGCAG GTAGGCGCTT ACGAGGAGGT CAAACGCGGG CGCTGCCCTC GGGAGAGCTG | 540 |
| AACACTTTCC AGGCAGTTTT GGCATCTGAT GGGTCTGATA GCTACGCCCT CTTTCTTTAT | 600 |
| CCTGCCAACG GCCTGCAGTT CCTTGGAACC CGCCCCAAAG AGTCTTACAA TGTCCAGCTT | 660 |
| CAGCTTCCAG CTCGGGTGGG CTTCTGCCGA GGGGAGGCTG ATGATCTGAA GTCAGAAGGA | 720 |
| CCATATTTCA GCTTGACTAG CACTGAACAG TCTGTGAAAA ATCTCTATCA ACTAAGCAAC | 780 |
| CTGGGGATCC CTGGAGTGTG GGCTTTCCAT ATCGGCAGCA CTTCCCCGTT GGACAATGTC | 840 |
| AGGCCAGCTG CAGTTGGAGA CCTTTCCGCT GCCCACTCTT CTGTTCCCCT GGGACGTTCC | 900 |
| TTCAGCCATG CTACAGCCCT GGAAAGTGAC TATAATGAGG ACAATTTGGA TTACTACGAT | 960 |
| GTGAATGAGG AGGAAGCTGA ATACCTTCCG GGTGAACCAG AGGAGGCATT GAATGGCCAC | 1020 |
| AGCAGCATTG ATGTTTCCTT CCAATCCAAA GTGGATACAA AGCCTTTAGA GGAATCTTCC | 1080 |
| ACCTTGGATC CTCACACCAA AGAAGGAACA TCTCTGGGAG AGGTAGGGGG CCCAGATTTA | 1140 |
| AAAGGCCAAG TTGAGCCCTG GGATGAGAGA GAGACCAGAA GCCCAGCTCC ACCAGAGGTA | 1200 |
| GACAGAGATT CACTGGCTCC TTCCTGGGAA ACCCCACCAC CGTACCCCGA AAACGGAAGC | 1260 |
| ATCCAGCCCT ACCCAGATGG AGGGCCAGTG CCTTCGGAAA TGGATGTTCC CCCAGCTCAT | 1320 |
| CCTGAAGAAG AAATTGTTCT TCGAAGTTAC CCTGCTTCAG GTCACACTAC ACCCTTAAGT | 1380 |
| CGAGGGACGT ATGAGGTGGG ACTGGAAGAC AACATAGGTT CCAACACCGA GGTCTTCACG | 1440 |
| TATAATGCTG CCAACAAGGA AACCTGTGAA CACAACCACA GACAATGCTC CCGGCATGCC | 1500 |
| TTCTGCACGG ACTATGCCAC TGGCTTCTGC TGCCACTGCC AATCCAAGTT TTATGGAAAT | 1560 |
| GGGAAGCACT GTCTGCCTGA GGGGGCACCT CACCGAGTGA ATGGGAAAGT GAGTGGCCAC | 1620 |
| CTCCACGTGG GCCATACACC CGTGCACTTC ACTGATGTGG ACCTGCATGC GTATATCGTG | 1680 |
| GGCAATGATG GCAGAGCCTA CACGGCCATC AGCCACATCC CACAGCCAGC AGCCCAGGCC | 1740 |
| CTCCTCCCCC TCACACCAAT TGGAGGCCTG TTTGGCTGGC TCTTTGCTTT AGAAAAACCT | 1800 |
| GGCTCTGAGA ACGGCTTCAG CCTCGCAGGT GCTGCCTTTA CCCATGACAT GGAAGTTACA | 1860 |
| TTCTACCCGG GAGAGGAGAC GGTTCGTATC ACTCAAACTG CTGAGGGACT TGACCCAGAG | 1920 |
| AACTACCTGA GCATTAAGAC CAACATTCAA GGCCAGGTGC CTTACGTCCC AGCAAATTTC | 1980 |
| ACAGCCCACA TCTCTCCCTA CAAGGAGCTG TACCACTACT CCGACTCCAC TGTGACCTCT | 2040 |
| ACAAGTTCCA GAGACTACTC TCTGACTTTT GGTGCAATCA ACCAAACATG GTCCTACCGC | 2100 |
| ATCCACCAGA ACATCACTTA CCAGGTGTGC AGGCACGCCC CCAGACACCC GTCCTTCCCC | 2160 |
| ACCACCCAGC AGCTGAACGT GGACCGGGTC TTTGCCTTGT ATAATGATGA AGAAAGAGTG | 2220 |
| CTTAGATTTG CTGTGACCAA TCAAATTGGC CCGGTCAAAG AAGATTCAGA CCCCACTCCG | 2280 |
| GTGAATCCTT GCTATGATGG GAGCCACATG TGTGACACAA CAGCACGGTG CCATCCAGGG | 2340 |
| ACAGGTGTAG ATTACACCTG TGAGTGCGCA TCTGGGTACC AGGGAGATGG ACGGAACTGT | 2400 |
| GTGGATGAAA ATGAATGTGC AACTGGCTTT CATCGCTGTG GCCCCAACTC TGTATGTATC | 2460 |
| AACTTCCCTG GAAGCTACAG GTGTGAGTGC CGGAGTGGTT ATGAGTTTGC AGATGACCGG | 2520 |
| CATACTTGCA TCTTGATCAC CCCACCTGCC AACCCCTGTG AGGATGGCAG TCATACCTGT | 2580 |
| GCTCCTGCTG GGCAGGCCCG GTGTGTTCAC CATGGAGGCA GCACGTTCAG CTGTGCCTGC | 2640 |
| CTGCCTGGTT ATGCCGGCGA TGGGCACCAG TGCACTGATG TAGATGAATG CTCAGAAAAC | 2700 |
| AGATGTCACC CTGCAGCTAC CTGCTACAAT ACTCCTGGTT CCTTCTCCTG CCGTTGTCAA | 2760 |
| CCCGGATATT ATGGGGATGG ATTTCAGTGC ATACCTGACT CCACCTCAAG CCTGACACCC | 2820 |
| TGTGAACAAC AGCAGCGCCA TGCCCAGGCC CAGTATGCCT ACCCTGGGGC CCGGTTCCAC | 2880 |
| ATCCCCCAAT GCGACGAGCA GGGCAACTTC CTGCCCCTAC AGTGTCATGG CAGCACTGGT | 2940 |
| TTCTGCTGGT GCGTGGACCC TGATGGTCAT GAAGTTCCTG GTACCCAGAC TCCACCTGGC | 3000 |
| TCCACCCCGC CTCACTGTGG ACCATCACCA GAGCCCACCC AGAGGCCCCC GACCATCTGT | 3060 |
| GAGCGCTGGA GGGAAAACCT GCTGGAGCAC TACGGTGGCA CCCCCCGAGA TGACCAGTAC | 3120 |
| GTGCCCCAGT GCGATGACCT GGGCCACTTC ATCCCCCTGC AGTGCCACGG AAAGAGCGAC | 3180 |
| TTCTGCTGGT GTGTGGACAA AGATGGCAGA GAGGTGCAGG GCACCCGCTC CCAGCCAGGC | 3240 |
| ACCACCCCTG CGTGTATACC CACCGTCGCT CCACCCATGG TCCGGCCCAC GCCCCGGCCA | 3300 |
| GATGTGACCC CTCCATCTGT GGGCACCTTC CTGCTCTATA CTCAGGGCCA GCAGATTGGC | 3360 |
| TACTTACCCC TCAATGGCAC CAGGCTTCAG AAGGATGCAG CTAAGACCCT GCTGTCTCTG | 3420 |
| CATGGCTCCA TAATCGTGGG AATTGATTAC GACTGCCGGG AGAGGATGGT GTACTGGACA | 3480 |
| GATGTTGCTG GACGGACAAT CAGCCGTGCC GGTCTGGAAC TGGGAGCAGA GCCTGAGACG | 3540 |
| ATCGTGAATT CAGGTCTGAT AAGCCCTGAA GGACTTGCCA TAGACCACAT CCGCAGAACA | 3600 |
| ATGTACTGGA CGGACAGTGT CCTGGATAAG ATAGAGAGCG CCCTGCTGGA TGGCTCTGAG | 3660 |
| CGCAAGGTCC TCTTCTACAC AGATCTGGTG AATCCCCGTG CCATCGCTGT GGATCCAATC | 3720 |
| CGAGGCAACT TGTACTGGAC AGACTGGAAT AGAGAAGCTC CTAAAATTGA AACGTCATCT | 3780 |
| TTAGATGGAG AAAACAGAAG AATTCTGATC AATACAGACA TTGGATTGCC CAATGGCTTA | 3840 |
| ACCTTTGACC CTTTCTCTAA ACTGCTCTGC TGGGCAGATG CAGGAACCAA AAAACTGGAG | 3900 |
| TGTACACTAC CTGATGGAAC TGGACGGCGT GTCATTCAAA ACAACCTCAA GTACCCCTTC | 3960 |
| AGCATCGTAA GCTATGCAGA TCACTTCTAC CACACAGACT GGAGGAGGGA TGGTGTTGTA | 4020 |
| TCAGTAAATA AACATAGTGG CCAGTTTACT GATGAGTATC TCCCAGAACA ACGATCTCAC | 4080 |
| CTCTACGGGA TAACTGCAGT CTACCCCTAC TGCCCAACAG GAAGAAAGTA AGTACAGTAA | 4140 |
| TGTAAAGGAA GACTTGGAGT TTACAATCAG AACCTGGACC CTAAAGAACA GTGACTGCAA | 4200 |
| AGGCAAAGAA AGTAAAAAAG GAATTGGCCA TTAGACGTTC CTGAGCATCC AAGATGAACA | 4260 |
| TTTTGTAGTG CAAAAAGACT TTTGTGAAAA GCTGATACCT CAATCTTTAC TACTGTATTT | 4320 |
| TTAAAAATGA AGGTTGTTAT TGCAAGTTTA AAAAGGTAAC AGAATTTTAA CTGTTGCTTA | 4380 |
| TTAAAGCAAC TTCTTGTAAA CATTTATCAT TAATATTTAA AAGATCAAAT TCATTCAACT | 4440 |
| AAGAATTAGA GTTTAAGACT CTAAACCTGA TTTTTGCCAT GGATTCCTTC TGGCCAAGAA | 4500 |
| ATTAAAGCAC ATGTGATCAA TATAACAATA TAATCCTAAA CCTTGACAGT TGGAGAAGCC | 4560 |
| AATGCAGAAC TGATGGGAAA GGACCAATTA TTTATAGTTT CCCAACAAAA GTTCTAAGAT | 4620 |
| TTTTTACCTC TGCATCAGTG CATTTCTATT TATATCAAAA GGTGCTAAAA TGATTCAATT | 4680 |
| TGCATTTTCT GATCCTGTAG TGCCTCTATA GAAGTACCCA CAGAAAGTAA AGTATCACAT | 4740 |
| TTATAAATAC CAAAGATGTA ACAATTTTAA AATTTTCTAG ATTACTCCAA TAAAGTGTTT | 4800 |
| TAAGTTTAAA AAAAAAAAAA AAAAAAAAA | |

Seq ID No: 117 Protein sequence:
Protein Accession #: NP_031387.1

| | |
|---|---|
| 1 11 21 31 41 51 | |
| \| \| \| \| \| \| | |
| MEGDRVAGRP VLSSLPVLLL LQLLMLRAAA LHPDELFPHG ESWWDQLLQE GDDVKLSRGE | 60 |
| AGESPALLTK PDSATSTWAP TASSPLRTSP GKRSMWTMIS PPTSRPSPLF WRTSTRATAE | 120 |
| AESCTERTPP PQCWAWPPAM CALASRALRA FYPHPRLPGH LGAGRRLRCG QTRALPSGEL | 180 |
| NTFQAVLASD GSDSYALFLY PANGLQFLGT RPKESYNVQL QLPARVGFCR GEADDLKSEG | 240 |
| PYFSLTSTEQ SVKNLYQLSN LGIPGVWAFH IGSTSPLDNV RPAAVGDLSA AHSSVPLGRS | 300 |
| FSHATALESD YNEDNLDYYD VNEEEAEYLP GEPEEALNGH SSIDVSFQSK VDTKPLEESS | 360 |
| TLDPHTKEGT SLGEVGGPDL KGQVEPWDER ETRSPAPPEV DRDSLAPSWE TPPPYPENGS | 420 |
| IQPYPDGGPV PSEMDVPPAH PEEETVLRSY PASGHTTPLS RGTYEVGLED NIGSNTEVFT | 480 |
| YNAANKETCE HNHRQCSRHA FCTDYATGFC CHCQSKFYGN GKHCLPEGAP HRVNGKVSGH | 540 |
| LHVGNTPVHF TDVDLHAYIV GNDGRAYTAI SNIPQPAAQA LLPLTPIGGL FGWLFALEKP | 600 |
| GSENGFSLAG AAFTHDMEVT FYPGEETVRI TQTAEGLDPE NYLSIKTNIQ GQVPYVPANF | 660 |
| TAHISPYKEL YHYSDSTVTS TSSRDYSLTF GAINQTWSYR IHQNITYQVC RHAPRHPSFP | 720 |
| TTQQLNVDRV FALYNDEERV LREAVTNQIG PVKEDSDPTP VNPCYDGSHM CDTTARCHPG | 780 |
| TGVDYTCECA SGYQGDGRNC VDENECATGF HRCGPNSVCI NLPGSYRCEC RSGYEFADDR | 840 |
| HTCILITPPA NPCEDGSHTC APAGQARCVH HGGSTFSCAC LPGYAGDGHQ CTDVDECSEN | 900 |
| RCHPAATCYN TPGSFSCRCQ PGYYGDGFQC IPDSTSSLTP CEQQQRHAQA QYAYPGARFH | 960 |
| IPQCDEQGNF LPLQCHGSTG FCWCVDPDGH EVPGTQTPPG STPPHCGPSP EPTQRPPTIC | 1020 |
| ERWRENLLEH YGGTPRDDQY VPQCDDLGHF IPLQCHGKSD FCWCVDKDGR EVQGTRSQPG | 1080 |
| TTPACIPTVA PPMVRPTPRP DVTPPSVGTF LLYTQGQQIG YLPLNGTRLQ KDAAKTLLSL | 1140 |
| HGSIIVGIDY DCRERMVYWT DVAGRTISRA GLELGAEPET IVNSGLISPE GLAIDHIRRT | 1200 |
| MYWTDSVLDK IESALLDGSE RKVLFYTDLV NPRAIAVDPI RGNLYWTDWN REAPKIETSS | 1260 |
| LDGENRRILI NTDIGLPNGL TFDPFSKLLC WADAGTKKLE CTLPDGTGRR VIQNNLKYPF | 1320 |
| SIVSYADHFY HTDWRRDGVV SVNKESGQFT DEYLPEQRSH LYGITAVYPY CPTGRK | |

Seq ID NO: 118 DNA sequence:
Nucleic Acid Accession #: NM_003088.1
Coding sequence: 112-1593 (underlined sequences correspond to start
and stop codons)

| | |
|---|---|
| 1 11 21 31 41 51 | |
| \| \| \| \| \| \| | |
| GCGGAGGGTG CGTGCGGGCC GCGGCAGCCG AACAAAGGAG CAGGGGCGCC GCCGCAGGGA | 60 |
| CCCGCCACCC ACCTCCCGGG GCCGCGCAGC GGCCTCTCGT CTACTGCCAC CATGACCGCC | 120 |
| AACGGCACAG CCGAGGCGGT GCAGATCCAG TTCGGCCTCA TCAACTGCGG CAACAAGTAC | 180 |
| CTGACGGCCG AGGCGTTCGG GTTCAAGGTG AACGCGTCCG CCAGCAGCCT GAAGAAGAAG | 240 |
| CAGATCTGGA CGCTGGAGCA GCCCCCTGAC GAGGCGGGCA GCGCGGCCGT GTGCCTGCGC | 300 |
| AGCCACCTGG GCCGCTACCT GGCGGCGGAC AAGGACGGCA ACGTGACCTG CGAGCGCGAG | 360 |
| GTGCCCGGTC CCGACTGCCG TTTCCTCATC GTGGCGCACG ACGACGGTCG CTGGTCGCTG | 420 |
| CAGTCCGAGG CGCACCGGCG CTACTTCGGC GGCACCGAGG ACCGCCTGTC CTGCTTCGCC | 480 |
| CAGACGGTGT CCCCCGCCGA GAAGTGGAGC GTGCACATCG CCATGCACCC TCAGGTCAAC | 540 |
| ATCTACAGTG TCACCCCTAA GCGCTACGCG CACCTGAGCG CGCGGCCGGC CGACQAGATC | 600 |
| GCCGTGGACC GCGACGTGCC CTGGGGCGTC GACTCGCTCA TCACCCTCGC CTTCCAGGAC | 660 |
| CAGCGCTACA GCGTCCAGAC CGCCGACCAC CGCTTCCTGC GCCACGACGG GCGCCTGGTG | 720 |
| GCGCGCCCCG AGCCGGCCAC TGGCTACACG CTGGAGTTCC GCTCCGGCAA GGTGGCCTTC | 780 |
| CGCGACTGCG AGGGCCGTTA CCTGGCGCCG TCGGGGCCCA GCGGCACGCT CAAGGCGGGC | 840 |
| AAGGCCACCA AGGTGGGCAA GGACGAGCTC TTTGCTCTGG AGCAGAGCTG CGCCCAGGTC | 900 |
| GTGCTGCAGG CGGCCAACGA GAGGAACGTG TCCACGCGCC AGGGTATGGA CCTGTCTGCC | 960 |
| AATCAGGACG AGGAGACCGA CCAGGAGACC TTCCAGCTGG AGATCGACCG CGACACCAAA | 1020 |
| AAGTGTGCCT TCCGTACCCA CACGGGCAAG TACTGGACGC TGACGGCCAC CGGGGGCGTG | 1080 |
| CAGTCCACCG CCTCCAGCAA GAATGCCAGC TGCTACTTTG ACATCGAGTG GCGTGACCGG | 1140 |
| CGCATCACAC TGAGGGCGTC CAATGGCAAG TTTGTGACCT CCAAGAAGAA TGGGCAGCTG | 1200 |
| GCCGCCTCGG TGGAGACAGC AGGGGACTCA GAGCTCTTCC TCATGAAGCT CATCAACCGC | 1260 |
| CCCATCATCG TGTTCCGCGG GGAGCATGGC TTCATCGGCT GCCGCAAGGT CACGGGCACC | 1320 |
| CTGGACGCCA ACCGCTCCAG CTATGACGTC TTCCAGCTGG AGTTCAACGA TGGCGCCTAC | 1380 |
| AACATCAAAG ACTCCACAGG CAAATACTGG ACGGTGGGCA GTGACTCCGC GGTCACCAGC | 1440 |
| AGCGGCGACA CTCCTGTGGA CTTCTTCTTC GAGTTCTGCG ACTATAACAA GGTGGCCATC | 1500 |
| AAGGTGGGCG GGCGCTACCT GAAGGGCGAC CACGCAGGCG TCCTGAAGGC CTCGGCGGAA | 1560 |
| ACCGTGGACC CCGCCTCGCT CTGGGAGTAC TAGGGCCGGC CCGTCCTTCC CCGCCCCTGC | 1620 |
| CCACATGGCG GCTCCTGCCA ACCCTCCCTG CTAACCCCTT CTCCGCCAGG TGGGCTCCAG | 1680 |
| GGCGGGAGGC AAGCCCCCTT GCCTTTCAAA CTGGAAACCC CAGAGAAAAC GGTGCCCCCA | 1740 |
| CCTGTCGCCC CTATGGACTC CCCACTCTCC CCTCCGCCCG GGTTCCCTAC TCCCCTCGGG | 1800 |
| TCAGCGGCTG CGGCCTGGCC CTGGGAGGGA TTTCAGATGC CCCTGCCCTC TTGTCTGCCA | 1860 |
| CGGGGCGAGT CTGGCACCTC TTTCTTCTGA CCTCAGACGG CTCTGAGCCT TATTTCTCTG | 1920 |
| GAAGCGGCTA AGGGAGGGTT GGGGGCTGGG AGCCCTGGGC GTGTAGTGTA ACTGGAATCT | 1980 |
| TTTGCCTCTC CCAGCCACCT CCTCCCAGCC CCCCAGGAGA GCTGGGCACA TGTCCCAAGC | 2040 |
| CTGTCAGTGG CCCTCCCTGG TGCACTGTCC CCGAAACCCC TGCTTGGGAA GGGAAGCTGT | 2100 |
| CGGGAGGGCT AGGACTGACC CTTGTGGTGT TTTTTTGGGT GGTGGCTGGA AACAGCCCCT | 2160 |
| CTCCCACGTG GGAGAGGCTC AGCCTGGCTC CCTTCCCTGG AGCGGCAGGG CGTGACGGCC | 2220 |
| ACAGGGTCTG CCCGCTGCAC GTTCTGCCAA GGTGGTGGTG GCGGGCGGGT AGGGGTGTGG | 2280 |
| GGCCCGTCTT CCTCCTGTCT CTTTCCTTTC ACCCTAGCCT GACTGGAAGC AGAAAATGAC | 2340 |
| CAAATCAGTA TTTTTTTTAA TGAAATATTA TTGCTGGAGG CGTCCCAGGC AAGCCTGGCT | 2400 |
| GTAGTAGCGA GTGATCTGGC GGGGGGCGTC TCAGCACCCT CCCCAGGGGG TGCATCTCAG | 2460 |
| CCCCCTCTTT CCGTCCTTCC CGTCCAGCCC CAGCCCTGGG CCTGGGCTGC CGACACCTGG | 2520 |
| GCCAGAGCCC CTGCTGTGAT TGGTGCTCCC TGGGCCTCCC GGGTGGATGA AGCCAGGCGT | 2580 |
| CGCCCCCTCC GGGAGCCCTG GGGTGAGCCG CCGGGGCCCC CCTGCTGCCA GCCTCCCCCG | 2640 |
| TCCCCAACAT GCATCTCACT CTGGGTGTCT TGGTCTTTTA TTTTTTGTAA GTGTCATTTG | 2700 |
| TATAACTCTA AACGCCCATG ATAGTAGCTT CAAACTGGAA ATAGCGAAAT AAAATAACTC | 2760 |
| AGTCTGC | |

Seq ID No: 119 protein sequence:
Protein Accession #: NP_003079.1

| | |
|---|---|
| 1 11 21 31 41 51 | |
| \| \| \| \| \| \| | |
| MTANGTAEAV QIQFGLINCG NKYLTAEAFG FKVNASASSL KKKQIWTLEQ PPDSAGSAAV | 60 |
| CLRSHLGRYL AADKDGNVTC EREVPCPDCR FLIVAHDDGR WSLQSEAHRR YFGGTEDRLS | 120 |
| CFAQTVSPAE KWSVHIAMHP QVNIYSVTRK RYAHLSARPA DEIAVDRDVP WGVDSLITLA | 180 |
| FQDQRYSVQT ADHRFLRHDG RLVARPEPAT GYTLSFRSGK VAFRGCEGRY LAPSGPSGTL | 240 |
| KAGKATKVGK DELFALEQSC AQVVLQAANE RNVSTRQGMD LSANQDEETD QETFQLEIDR | 300 |
| DTKKCAFRTH TGKYWTLTAT GGVQSTASSK NASCYFDIEW RDRRITLRAS NGKFVTSKKN | 360 |
| GQLAASVETA GDSELFLMKL INRPIIVFRG EHGFIGCRKV TGTLDANRSS YDVFQLEFND | 420 |
| GAYNIKDSTG KYWTVGSDSA VTSSGDTPVD FFFEFCDYNK VAIKVGGRYL KGDHAGVLKA | 480 |
| SAETVDPASL WEY | |

Seq ID NO: 120 DNA sequence:
Nucleic Acid Accession #: NM_006404.1
Coding sequence: 25-741 (underlined sequences correspond to start and stop codons)

| | |
|---|---|
| 1 11 21 31 41 51 | |
| \| \| \| \| \| \| | |
| CAGGTCCGGA GCCTCAACTT CAGGATGTTG ACAACATTGC TGCCGATACT GCTGCTGTCT | 60 |
| GGCTGGGCCT TTTGTAGCCA AGACGCCTCA GATGGCCTCC AAAGACTTCA TATGCTCCAG | 120 |
| ATCTCCTACT TCCGCGACCC CTATCACGTG TGGTACCAGG GCAACGCGTC GCTGGGGGGA | 180 |
| CACCTAACGC ACGTGCTGGA AGGCCCAGAC ACCAACACCA CGATCATTCA GCTGCAGCCC | 240 |
| TTGCAGGAGC CCGAGAGCTG GGCGCGCACG CAGAGTGGCC TGCAGTCCTA CCTGCTCCAG | 300 |
| TTCCACGGCC TCGTGCGCCT GGTGCACCAG GAGCGGACCT TGGCCTTTCC TCTGACCATC | 360 |
| CGCTGCTTCC TGGGCTGTGA GCTGCCTCCC GAGGGCTCTA GAGCCCATGT CTTCTTCGAA | 420 |
| GTGGCTGTGA ATGGGAGCTC CTTTGTGAGT TTCCGGCCGG AGAGAGCCTT GTGGCAGGCA | 480 |
| GACACCCAGG TCACCTCCGG AGTGGTCACC TTCACCCTGC AGCAGCTCAA TGCCTACAAC | 540 |
| CGCACTCGGT ATGAACTGCG GGAATTCCTG GAGGACACCT GTGTGCAGTA TGTGCAGAAA | 600 |
| CATATTTCCG CGGAAAACAC GAAAGGGAGC CAAACAAGCC GCTCCTACAC TTCGCTGGTC | 660 |
| CTGGGCGTCC TGGTGGGCGG TTTCATCATT GCTGGTGTGG CTGTAGGCAT CTTCCTGTGC | 720 |
| ACAGGTGGAC GGCGATGTTA ATTACTCTCC AGCCCCGTCA GAAGGGGCTG GATTGATGGA | 780 |
| GGCTGGCAAG GGAAAGTTTC GGCTCACTGT GAAGCCAGAC TCCCCAACTG AAACACCAGA | 840 |
| AGGTTTGGAG TGACAGCTCC TTTCTTCTCC CACATCTGCC CACTGAAGAT TTGAGGGAGG | 900 |
| GGAGATGGAG AGGAGAGGTG GACAAAGTAC TTGGTTTGCT AAGAACCTAA GAACGTGTAT | 960 |
| GCTTTGCTGA ATTAGTCTGA TAAGTGAATG TTTATCTATC TTTGTGGAAA ACAGATAATG | 1020 |
| GAGTTGGGGC AGGAAGCCTA TGCGCCATCC TCCAAAGACA GACAGAATCA CCTGAGGCGT | 1080 |
| TCAAAAGATA TAACCAAATA AACAAGTCAT CCACAATCAA AATACAACAT TCAATACTTC | 1140 |
| CAGGTGTGTC AGACTTGGGA TGGGACGCTG ATATAATAGG GTAGAAAGAA GTAACACGAA | 1200 |
| GAAGTGGTGG AAATGTAAAA TCCAAGTCAT ATGGCAGTGA TCAATTATTA ATCAATTAAT | 1260 |
| AATATTAATA AATTTCTTAT ATTT | |

Seq ID No: 121 Protein sequence:
Protein Accession #: NP_006395.1

| | |
|---|---|
| 1 11 21 31 41 51 | |
| \| \| \| \| \| \| | |
| MLTTLLPILL LSGWAFCSQD ASDGLQRLHM LQISYFRDPY HVWYQGNASL GGHLTHVLEG | 60 |
| PDTNTTIIQL QPLQEPESWA RTQSGLQSYL LQFHGLVRLV HQERTLAFPL TIRCFLGCEL | 120 |
| PPEGSRAHVF FEVAVNGSSF VSFRPERALW QADTQVTSGV VTFTLQQLNA YNRTRYELRE | 180 |
| FLEDTCVQYV QKHISAENTK GSQTSRSYTS LVLGVLVGGF IIAGVAVGIF LCTGGRRC | |

Seq ID NO: 122 DNA sequence:
Nucleic Acid Accession #: none found
Coding sequence: 2-505 (underlined sequences correspond to start and
stop codons)

| | |
|---|---|
| 1 11 21 31 41 51 | |
| \| \| \| \| \| \| | |
| CGAGAAGCTG GGAGAGACAC CACTTGTCCC TGAACAAGAC AATTCAGTAA CATCTATTCC | 60 |
| TGAGATTCCT CGATGGGGAT CACAGAGCAC GATGTCTACC CTTCAAATGT CCCTTCAAGC | 120 |
| CGAGTCAAAG GCCACTATCA CCCCATCAGG GAGCGTGATT TCCAAGTTTA ATTCTACGAC | 180 |
| TTCCTCTGCC ACTCCTCAGG CTTTCGACTC CTCCTCTGCC GTGGTCTTCA TATTTGTGAG | 240 |
| CACAGCAGTA GTAGTGTTGG TGATCTTGAC CATGACAGTA CTGGGGCTTG TCAAGCTCTG | 300 |
| CTTTCACGAA AGCCCCTCTT CCCAGCCAAG GAAGGAGTCT ATGGGCCCGC CGGGCCTGGA | 360 |
| GAGTGATCCT GAGCCCGCTG CTTTGGGCTC CAGTTCTGCA CATTGCACAA ACAATGGGGT | 420 |
| GAAAGTCGGG GACTGTGATC TGCGGGACAG AGCAGAGGGT GCCTTGCTGG CGGAGTCCCC | 480 |
| TCTTGGCTCT AGTGATGCAT AGGGAAACAG GGGACATGGG CACTCCTGTG AACAGTTTTT | 540 |
| CACTTTTGAT GAAACGGGGA ACCAAGAGGA ACTTACTTGT GTAACTGACA ATTTCTGCAG | 600 |
| AAATCCCCCT TCCTCTAAAT TCCCTTTACT CCACTGAGGA GCTAAATCAG AACTGCACAC | 660 |
| TCCTTCCCTG ATGATAGAGG AAGTGGAAGT GCCTTTAGGA TGGTGATACT GGGGGACCGG | 720 |
| GTAGTGCTGG GGAGAGATAT TTTCTTATGT TTATTCGGAG AATTTGGAGA AGTGATTGAA | 780 |
| CTTTTCAAGA CATTGGAAAC AAATAGAACA CAATATAATT TACATTAAAA AATAATTTCT | 840 |
| ACCAAAATGG AAAGGAAATG TTCTATGTTG TTCAGGCTAG GAGTATATTG GTTCGAAATC | 900 |
| CCAGGGAAAA AAATAAAAAT AAAAAATTAA AGGATTGTTG ATAAAA | |

Seq ID No: 123 Protein sequence:
Protein Accession #: none found

| | |
|---|---|
| 1 11 21 31 41 51 | |
| \| \| \| \| \| \| | |
| EKLGETPLVP EQDNSVTSIP EIPRWGSQST MSTLQMSLQA ESKATITPSG SVISKFNSTT | 60 |
| SSATPQAFDS SSAVVFIFVS TAVVVLVILT MTVLGLVKLC FHESPSSQPR KESMGPPGLE | 120 |
| SDPEPAALGS SSAHCTNNGV KVGDCDLRDR AEGALLAESP LGSSDA | |

Seq ID NO: 124 DNA sequence:
Nucleic Acid Accession #: NM_006500.1
Coding sequence: 27-1967 (underlined sequences correspond to start and stop codons)

| | |
|---|---|
| 1 11 21 31 41 51 | |
| \| \| \| \| \| \| | |
| ACTTGCGTCT CGCCCTCCGG CCAAGCATGG GGCTTCCCAG GCTGGTCTGC GCCTTCTTGC | 60 |
| TCGCCGCCTG CTGCTGCTGT CCTCGCGTCG CGGGTGTGCC CGGAGAGGCT GAGCAGCCTG | 120 |
| CGCCTGAGCT GGTGGAGGTG GAAGTGGGCA GCACAGCCCT TCTGAAGTGC GGCCTCTCCC | 180 |
| AGTCCCAAGG CAACCTCAGC CATGTCGACT GGTTTTCTGT CCACAAGGAG AAGCGGACGC | 240 |
| TCATCTTCCG TGTGCGCCAG GGCCAGGGCC AGAGCGAACC TGGGGAGTAC GAGCAGCGGC | 300 |
| TCAGCCTCCA GGACAGAGGG GCTACTCTGG CCCTGACTCA AGTCACCCCC CAAGACGAGC | 360 |
| GCATCTTCTT GTGCCAGGGC AAGCGCCCTC GGTCCCAGGA GTACCGCATC CAGCTCCGCG | 420 |
| TCTACAAAGC TCCGGAGGAG CCAAACATCC AGGTCAACCC CCTGGGCATC CCTGTGAACA | 480 |
| GTAAGGAGCC TGAGGAGGTC GCTACCTGTG TAGGGAGGAA CGGGTACCCC ATTCCTCAAG | 540 |
| TCATCTGGTA CAAGAATGGC CGGCCTCTGA AGGAGGAGAA GAACCGGGTC CACATTCAGT | 600 |
| CGTCCCAGAC TGTGGAGTCG AGTGGTTTGT ACACCTTGCA GAGTATTCTG AAGGCACAGC | 660 |
| TGGTTAAAGA AGACAAAGAT GCCCAGTTTT ACTGTGAGCT CAACTACCGG CTGCCCAGTG | 720 |
| GGAACCACAT GAAGGAGTCC AGGGAAGTCA CCGTCCCTGT TTTCTACCCG ACAGAAAAAG | 780 |
| TGTGGCTGGA AGTGGAGCCC GTGGGAATGC TGAAGGAAGG GGACCGCGTG GAAATCAGGT | 840 |
| GTTTGGCTGA TGGCAACCCT CCACCACACT TCAGCATCAG CAAGCAGAAC CCCAGCACCA | 900 |
| GGGAGGCAGA GGAAGAGACA ACCAACGACA ACGGGGTCCT GGTGCTGGAG CCTGCCCGGA | 960 |
| AGGAACACAG TGGGCGCTAT GAATGTCAGG CCTGGAACTT GGACACCATG ATATCGCTGC | 1020 |
| TGAGTGAACC ACAGGAACTA CTGGTGAACT ATGTGTCTGA CGTCCGAGTG AQTCCCGCAG | 1080 |
| CCCCTGAGAG ACAGGAAGGC AGCAGCCTCA CCCTGACCTG TGAGGCAGAG AGTAGCCAGG | 1140 |
| ACCTCGAGTT CCAGTGGCTG AGAGAAGAGA CAGACCAGGT GCTGCAAAGG GGGCCTGTGC | 1200 |
| TTCAGTTGCA TGACCTGAAA CGGGAGGCAG GAGGCGGCTA TCGCTGCGTG GCGTCTGTGC | 1260 |
| CCAGCATACC CGCCCTGAAC CGCACACAGC TGGTCAAGCT GGCCATTTTT GGCCCCCCTT | 1320 |
| GGATGGCATT CAAGGAGAGG AAGGTGTGGG TGAAAGAGAA TATGGTGTTG AATCTGTCTT | 1380 |
| GTGAAGCGTC AGGGCACCCC CGGCCCACCA TCTCCTGGAA CGTCAACGGC ACGGCAAGTG | 1440 |
| AACAAGACCA AGATCCACAG CGAGTCCTGA GCACCCTGAA TGTCCTCGTG ACCCCGGAGC | 1500 |
| TGTTGGAGAC AGGTGTTGAA TGCACGGCCT CCAACGACCT GGGCAAAAAC ACCAGCATCC | 1560 |
| TCTTCCTGGA GCTGGTCAAT TTAACCACCC TCACACCAGA CTCCAACACA ACCACTGGCC | 1620 |
| TCAGCACTTC CACTGCCAGT CCTCATACCA GAGCCAACAG CACCTCCACA GAGAGAAAGC | 1680 |
| TGCCGGAGCC GGAGAGCCGG GGCGTGGTCA TCGTGGCTGT GATTGTGTGC ATCCTGGTCC | 1740 |
| TGGCGGTGCT GGGCGCTGTC CTCTATTTCC TCTATAAGAA GGGCAAGCTG CCGTGCAGGC | 1800 |
| GCTCAGGGAA GCAGGAGATC ACGCTGCCCC CGTCTCGTAA GACCGAACTT GTAGTTGAAG | 1860 |
| TTAAGTCAGA TAAGCTCCCA GAAGAGATGG GCCTCCTGCA GGGCAGCAGC GGTGACAAGA | 1920 |
| GGGCTCCGGG AGACCAGGGA GAGAAATACA TCGATCTGAG GCATTAGCCC CGAATCACTT | 1980 |
| CAGCTCCCTT CCCTGCCTGG ACCATTCCCA GCTCCCTGCT CACTCTTCTC TCAGCCAAAG | 2040 |
| CCTCCAAAGG GACTAGAGAG AAGCCTCCTG CTCCCCTCAC CTGCACACCC CCTTTCAGAG | 2100 |
| GGCCACTGGG TTAGGACCTG AGGACCTCAC TTCGCCCTGC AAGCCGCTTT TCAGGGACCA | 2160 |
| GTCCACCACC ATCTCCTCCA CGTTGAGTGA AGCTCATCCC AAGCAAGGAG CCCCAGTCTC | 2220 |
| CCGAGCGGGT AGGAGAGTTT CTTGCAGAAC GTGTTTTTTC TTTACACACA TTATGGCTGT | 2280 |
| AAATACCTGG CTCCTGCCAG CAGCTGAGCT GGGTAGCCTC TCTGAGCTGG TTTCCTGCCC | 2340 |
| CAAAGGCTGG CTTCCACCAT CCAGGTGCAC CACTGAAGTG AGGACACACC GGAGCCAGGC | 2400 |
| GCCTGCTCAT GTTGAAGTGC GCTGTTCACA CCCGCTCCGG AGAGCACCCC AGCGGCATCC | 2460 |
| AGAAGCAGCT GCAGTGTTGC TGCCACCACC CTCCTGCTCG CCTCTTCAAA GTCTCCTGTG | 2520 |
| ACATTTTTTC TTTGGTCAGA AGCCAGGAAC TGGTGTCATT CCTTAAAAGA TACGTGCCGG | 2580 |
| GGCCAGGTGT GGTGGCTCAC GCCTGTAATC CCAGCACTTT GGGAGGCCGA GGCGGGCGGA | 2640 |
| TCACAAAGTC AGGACGAGAC CATCCTGGCT AACACGGTGA AACCCTGTCT CTACTAAAAA | 2700 |
| TACAAAAAAA AATTAGCTAG GCGTAGTGGT TGGCACCTAT AGTCCCAGCT ACTCGGAAGG | 2760 |
| CTGAAGCAGG AGAATGGTAT GAATCCAGGA GGTGGAGCTT GCAGTGAGCC GAGACCGTGC | 2820 |
| CACTGCACTC CAGCCTGGGC AACACAGCGA GACTCCGTCT CGAGGAAAAA AAAAGAAAAG | 2880 |
| ACGCGTACCT GCGGTGAGGA AGCTGGGCGC TGTTTTCGAG TTCAGGTGAA TTAGCCTCAA | 2940 |
| TCCCCGTGTT CACTTGCTCC CATAGCCCTC TTGATGGATC ACGTAAAACT GAAAGGCAGC | 3000 |
| GGGGAGCAGA CAAAGATGAG GTCTACACTG TCCTTCATGG GGATTAAAGC TATGGTTATA | 3060 |
| TTAGCACCAA ACTTCTACAA ACCAAGCTCA GGGCCCCAAC CCTAGAAGGG CCCAAATGAG | 3120 |
| AGAATGGTAC TTAGGGATGG AAAACGGGGC CTGGCTAGAG CTTCGGGTGT GTGTGTCTGT | 3180 |
| CTGTGTGTAT GCATACATAT GTGTGTATAT ATGGTTTTGT CAGGTGTGTA AATTTGCAAA | 3240 |
| TTGTTTCCTT TATATATGTA TGTATATATA TATATGAAAA TATATATATA TATGAAAAAT | 3300 |
| AAAGCTTAAT TGTCCCAGAA AATCATACAT TGCTTTTTTA TTCTACATGG GTACCACAGG | 3360 |
| AACCTGGGGG CCTGTGAAAC TACAACCAAA AGGCACACAA AACCGTTTCC AGTTGGCAGC | 3420 |
| AGAGATCAGG GGTTACCTCT GCTTCTGAGC AAATGGCTCA AGCTCTACCA GAGCAGACAG | 3480 |
| CTACCCTACT TTTCAGCAGC AAAACGTCCC GTATGACGCA GCACGAAGGG CCTGGCAGGC | 3540 |
| TGTTAGCAGG AGCTATGTCC CTTCCTATCG TTTCCGTCCA CTT | |

Seq ID No: 125 Protein sequence:
Protein Accession #: NP_006491.1

| | |
|---|---|
| 1 11 21 31 41 51 | |
| \| \| \| \| \| \| | |
| MGLPRLVCAF LLAACCCCPR VAGVPGEAEQ PAPELVEVEV GSTALLKCGL SQSQGNLSHV | 60 |
| DWFSVHKEKR TLIFRVRQGQ GQSEPGEYEQ RLSLQDRGAT LALTQVTPQD ERIFLCQGKR | 120 |
| PRSQEYRIQL RVYKAPEEPN IQVNPLGIPV NSKEPEEVAT CVGRNGYPIP QVIWYKNGRP | 180 |
| LKEEKNRVNI QSSQTVESSG LYTLQSILKA QLVKEDKDAQ FYCELNYRLP SGNHMKESRE | 240 |
| VTVPVFYPTE KVWLEVEPVG MLKEGDRVEI RCLADGNPPP HFSISKQNPS TREAEEETTN | 300 |
| DNGVLVLEPA RKEHSGRYEC QAWNLDTMIS LLSEPQELLV NYVSDVRVSP AAPERQEGSS | 360 |
| LTLTCEAESS QDLEFQWLRE STDQVLERGP VLQLHDLKRE AGGGYRCVAS VPSIPGLNRT | 420 |
| QLVKLAIFGP PWMAFKERKV WVKENMVLNL SCEASGHPRP TISWNVNGTA SEQDQDPQRV | 480 |
| LSTLNVLVTP ELLETGVECT ASNDLGKNTS ILFLELVNLT TLTPDSNTTT GLSTSTASPH | 540 |
| TRANSTSTER KLPEPESRGV VIVAVIVCIL VLAVLGAVLY FLYKKGKLPC RRSGKQEITL | 600 |
| PPSRKTELVV EVKSDKLPEE MGLLQGSSGD KRAPGDQGEK YIDLRH | |

Seq ID NO: 126 DNA sequence:
Nucleic Acid Accession #: NM_001955.1
Coding sequence: 337-975 (underlined sequences correspond to start and
stop codons)

| | |
|---|---|
| 1 11 21 31 41 51 | |
| \| \| \| \| \| \| | |
| GGAGCTGTTT ACCCCCACTC TAATAGGGGT TCAATATAAA AAGCCGGCAG AGAGCTGTCC | 60 |
| AAGTCAGACG CGCCTCTGCA TCTGCGCCAG GCGAACGGGT CCTGCGCCTC CTGCAGTCCC | 120 |
| AGCTCTCCAC CACCGCCGCG TGCGCCTGCA GACGCTCCGC TCGCTGCCTT CTCTCCTGGC | 180 |
| AGGCGCTGCC TTTTCTCCCC GTTAAAGGGC ACTTGGGCTG AAGGATCGCT TTGAGATCTG | 240 |
| AGGAACCCGC AGCGCTTTGA GGGACCTGAA GCTGTTTTTC TTCGTTTTCC TTTGGGTTCA | 300 |
| GTTTGAACGG GAGGTTTTTG ATCCCTTTTT TTCAGAATGG ATTATTTGCT CATGATTTTC | 360 |
| TCTCTGCTGT TTGTGGCTTG CCAAGGAGCT CCAGAAACAG CAGTCTTAGG CGCTGAGCTC | 420 |
| AGCGCGGTGG GTGAGAACGG CGGGGAGAAA CCCACTCCCA GTCCACCCTG GCGGCTCCGC | 480 |
| CGGTCCAAGC GCTGCTCCTG CTCGTCCCTG ATGGATAAAG AGTGTGTCTA CTTCTGCCAC | 540 |
| CTGGACATCA TTTGGGTCAA CACTCCCGAG CACGTTGTTC CGTATGGACT TGGAAGCCCT | 600 |
| AGGTCCAAGA GAGCCTTGGA GAATTTACTT CCCACAAAGG CAACAGACCG TGAGAATAGA | 660 |
| TGCCAATGTG CTAGCCAAAA AGACAAGAAG TGCTGGAATT TTTGCCAAGC AGGAAAAGAA | 720 |
| CTCAGGGCTG AAGACATTAT GGAGAAAGAC TGGAATAATC ATAAGAAAGG AAAAGACTGT | 780 |
| TCCAAGCTTG GGAAAAAGTG TATTTATCAG CAGTTAGTGA GAGGAAGAAA AATCAGAAGA | 840 |
| AGTTCAGAGG AACACCTAAG ACAAACCAGG TCGGAGACCA TGAGAAACAG CGTCAAATCA | 900 |
| TCTTTTCATG ATCCCAAGCT GAAAGGCAAG CCCTCCAGAG AGCGTTATGT GACCCACAAC | 960 |
| CGAGCACATT GGTGACAGAC TTCGGGGCCT GTCTGAAGCC ATAGCCTCCA CGGAGAGCCC | 1020 |
| TGTGGCCGAC TCTGCACTCT CCACCCTGGC TGGGATCAGA GCAGGAGCAT CCTCTGCTGG | 1080 |
| TTCCTGACTG GCAAAGGACC AGCGTCCTCG TTCAAAACAT TCCAAGAAAG GTTAAGGAGT | 1140 |
| TCCCCCAACC ATCTTCACTG GCTTCCATCA GTGGTAACTG CTTTGGTCTC TTCTTTCATC | 1200 |
| TGGGGATGAC AATGGACCTC TCAGCAGAAA CACACAGTCA CATTCGAATT C | |

Seq ID No: 127 Protein sequence:
Protein Accession #: NP_001946.1

| | |
|---|---|
| 1 11 21 31 41 51 | |
| \| \| \| \| \| \| | |
| MDYLLMIFSL LFVACQGAPE TAVLGAELSA VGENCCEKPT PSPPWRLRRS KRCSCSSLMD | 60 |
| KECVYFCHLD IIWVNTPEHV VPYGLGSPRS KRALENLLPT KATDRENRCQ CASQKDKKCW | 120 |
| NFCQACKELR AEDIMEKDWN NHKKGKDCSK LGKKCIYQQL VRGRKIRRSS EEHLRQTRSE | 180 |
| TMRNSVKSSF HDPKLKGKPS RERYVTHNRA HW | |

Seq ID NO: 128 DNA sequence:
Nucleic Acid Accession #: NM_001721.1
Coding sequence: 34-2061 (underlined sequences correspond to start and
stop codons)

| | |
|---|---|
| 1 11 21 31 41 51 | |
| \| \| \| \| \| \| | |
| GCAAGCACGG AACAAGCTGA GACGGATGAT AATATGGATA CAAAATCTAT TCTAGAAGAA | 60 |
| CTTCTTCTCA AAAGATCACA GCAAAAGAAG AAAATGTCAC CAAATAATTA CAAAGAACGG | 120 |
| CTTTTTGTTT TGACCAAAAC AAACCTTTCC TACTATGAAT ATGACAAAAT GAAAAGGGGC | 180 |
| AGCAGAAAAG GATCCATTGA AATTAAGAAA ATCAGATGTG TGGAGAAAGT AAATCTCGAG | 240 |
| GAGCAGACGC CTGTAGAGAG ACAGTACCCA TTTCAGATTG TCTATAAAGA TGGGCTTCTC | 300 |
| TATGTCTATG CATCAAATGA AGAGAGCCGA AGTCAGTGGT TGAAAGCATT ACAAAAAGAG | 360 |
| ATAAGGGGTA ACCCCCACCT GCTGGTCAAG TACCATAGTG GGTTCTTCGT GGACGGGAAG | 420 |
| TTCCTGTGTT GCCAGCAGAG CTGTAAAGCA GCCCCAGGAT GTACCCTCTG GGAAGCATAT | 480 |
| GCTAATCTGC ATACTGCAGT CAATGAAGAG AAACACAGAG TTCCCACCTT CCCAGACAGA | 540 |
| GTGCTGAAGA TACCTCGGGC AGTTCCTGTT CTCAAAATGG ATGCACCATC TTCAAGTACC | 600 |
| ACTCTAGCCC AATATGACAA CGAATCAAAG AAAAACTATG GCTCCCAGCC ACCATCTTCA | 660 |
| AGTACCAGTC TAGCGCAATA TGACAGCAAC TCAAAGAAAA TCTATGGCTC CCAGCCAAAC | 720 |
| TTCAACATGC AGTATATTCC AAGGGAAGAC TTCCCTGACT GGTGGCAAGT AAGAAAACTG | 780 |
| AAAAGTAGCA GCAGCAGTGA AGATGTTGCA AGCAGTAACC AAAAAGAAAG AAATGTGAAT | 840 |
| CACACCACCT CAAAGATTTC ATGGGAATTC CCTGAGTCAA GTTCATCTGA AGAAGAGGAA | 900 |
| AACCTGGATG ATTATGACTG GTTTGCTGGT AACATCTCCA GATCACAATC TGAACAGTTA | 960 |
| CTCAGACAAA AGGGAAAAGA AGGAGCATTT ATGGTTAGAA ATTCGAGCCA AGTGGGAATG | 1020 |
| TACACAGTGT CCTTATTTAG TAAGGCTGTG AATGATAAAA AAGGAACTGT CAAACATTAC | 1080 |
| CACGTGCATA CAAATGCTGA GAACAAATTA TACCTGGCAG AAAACTACTG TTTTGATTCC | 1140 |
| ATTCCAAAGC TTATTCATTA TCATCAACAC AATTCAGCAG GCATGATCAC ACGGCTCCGC | 1200 |
| CACCCTGTGT CAACAAAGGC CAACAAGGTC CCCGACTCTG TGTCCCTGGG AAATGGAATC | 1260 |
| TGGGAACTGA AAAGAGAAGA GATTACCTTG TTGAAGGAGC TGGGAAGTGG CCAGTTTGGA | 1320 |
| GTGGTCCAGC TGGGCAAGTG GAAGGGGCAG TATGATGTTG CTGTTAAGAT GATCAAGGAG | 1380 |
| GGCTCCATGT CAGAAGATGA ATTCTTTCAG GAGGCCCAGA CTATGATGAA ACTCAGCCAT | 1440 |
| CCCAAGCTGG TTAAATTCTA TGGAGTGTGT TCAAAGGAAT ACCCCATATA CATAGTGACT | 1500 |
| GAATATATAA GCAATGGCTG CTTGCTGAAT TACCTGAGGA GTCACGGAAA AGGACTTGAA | 1560 |
| CCTTCCCAGC TCTTAGAAAT GTGCTACGAT GTCTGTGAAG GCATGGCCTT CTTGGAGAGT | 1620 |
| CACCAATTCA TACACCGGGA CTTGGCTGCT CGTAACTGCT TGGTGGACAG AGATCTCTGT | 1680 |
| GTGAAAGTAT CTGACTTTGG AATGACAAGG TATGTTCTTG ATGACCAGTA TGTCAGTTCA | 1740 |
| GTCGGAACAA AGTTTCCAGT CAAGTGGTCA GCTCCAGAGG TGTTTCATTA CTTCAAATAC | 1800 |
| AGCAGCAAGT CAGACGTATG GGCATTTGGG ATCCTGATGT GGGAGGTGTT CAGCCTGGGG | 1860 |
| AAGCAGCCCT ATGACTTGTA TGACAACTCC CAGGTGGTTC TGAAGGTCTC CCAGGGCCAC | 1920 |
| AGGCTTTACC GGCCCCACCT GGCATCGGAC ACCATCTACC AGATCATGTA CAGCTGCTGG | 1980 |
| CACGAGCTTC CAGAAAAGCG TCCCACATTT CAGCAACTCC TGTCTTCCAT TGAACCACTT | 2040 |
| CGGGAAAAAG ACAAGCATTG AAGAAGAAAT TAGGAGTGCT GATAAGAATG AATATAGATG | 2100 |
| CTGGCCAGCA TTTTCATTCA TTTTAAGGAA AGTAGGAAGG CATAAGTAAT TTTAGCTAGT | 2160 |
| TTTTAATAGT GTTCTCTGTA TTGTCTATTA TTTAGAAATG AACAAGGCAG GAAACAAAAG | 2220 |
| ATTCCCTTGA AATTTAGATC AAATTAGTAA TTTTGTTTTA TGCTGCTCCT GATATAACAC | 2280 |
| TTTCCAGCCT ATAGCAGAAG CACATTTTCA GACTGCAATA TAGAGACTGT GTTCATGTGT | 2240 |
| AAACACTGAG CAGAACTGAA AAATTACTTA TTGGATATTC ATTCTTTTCT TTATATTGTC | 2400 |
| ATTGTCACAA CAATTAAATA TACTACCAAG TACAGAAATG TGGAAAAAAA AAACCG | |

Seq ID No: 129 Protein sequence:
Protein Accession #: NP_001712.1

| | |
|---|---|
| 1 11 21 31 41 51 | |
| \| \| \| \| \| \| | |
| MDTKSILEEL LLKRSQQKKK MSPNNYKERL FVLTKTNLSY YEYDKMKRGS RKGSIEIKKI | 60 |
| RCVEKVNLEE QTPVERQYPF QIVYKDGLLY VYASNEESRS QWLKALQKEI RGNPHLLVKY | 120 |
| HSGFFVDGKF LCCQQSCKAA PGCTLWEAYA NLHTAVNEEK HRVPTFPDRV LKIPRAVPVL | 180 |
| KMDAPSSSTT LAQYDNESKK NYGSQPPSSS TSLAQYDSNS KKIYGSQPNF NMQYIPREDF | 240 |
| PDWWQVRKLK SSSSSEDVAS SNQKERNVNH TTSKISWEFP ESSSSEEEEN LDDYDWFAGN | 300 |
| ISRSQSEQLL RQKGKEGAFM VRNSSQVGMY TVSLFSKAVN DKKGTVKHYH VHTNAENKLY | 360 |
| LAENYCFDSI PKLIHYHQHN SAGMITRLRH PVSTKANKVP DSVSLGNGIW ELKREEITLL | 420 |
| KELGSGQFGV VQLGKWKGQY DVAVKMIKEG SMSEDEFFQE AQTMMKLSHP KLVKFYGVCS | 480 |
| KEYPIYIVTE YISNGCLLNY LRSHGKGLEP SQLLEMCYDV CEGMAFLESH QFIHRDLAAR | 540 |
| NCLVDRDLCV KVSDFGMTRY VLDDQYVSSV GTKFPVKWSA PEVFHYFKYS SKSDVWAEGI | 600 |
| LMWEVFSLGK QPYDLYDNSQ VVLKVSQGHR LYRPHLASDT IYQIMYSCWH ELPEKRPTFQ | 660 |
| QLLSSIEPLR EKDKH | |

Seq ID NO: 130 DNA sequence:
Nucleic Acid Accession #: NM_012072.2
Coding sequence: 149-2107 (underlined sequences correspond to start
and stop codons)

| | |
|---|---|
| 1 11 21 31 41 51 | |
| \| \| \| \| \| \| | |
| AAAGCCCTCA GCCTTTGTGT CCTTCTCTGC GCCGGAGTGG CTGCAGCTCA CCCCTCAGCT | 60 |
| CCCCTTGGGG CCCAGCTGGG AGCCGAGATA GAAGCTCCTG TCGCCGCTGG GCTTCTCGCC | 120 |
| TCCCGCAGAG GGCCACACAG AGACCGGGAT GGCCACCTCC ATGGGCCTGC TGCTGCTGCT | 180 |
| GCTGCTGCTC CTGACCCAGC CCGGGGCGGG GACGGGAGCT GACACGGAGG CGGTGGTCTG | 240 |
| CGTGGGGACC GCCTGCTACA CGGCCCACTC GGGCAAGCTG AGCGCTGCCG AGGCCCAGAA | 300 |
| CCACTGCAAC CAGAACGGGG GCAACCTGGC CACTGTGAAG AGCAAGGAGG AGGCCCAGCA | 360 |
| CGTCCAGCGA GTACTGGCCC AGCTCCTGAG GCGGGAGGCA GCCCTGACGG CGAGGATGAG | 420 |
| CAAGTTCTGG ATTGGGCTCC AGCGAGAGAA GGGCAAGTGC CTGGACCCTA GTCTGCCGCT | 480 |
| GAAGGGCTTC AGCTGGGTGG GCGGGGGGGA GGACACGCCT TACTCTAACT GGCACAAGGA | 540 |
| GCTCCGGAAC TCGTGCATCT CCAAGCGCTG TGTGTCTCTG CTGCTGGACC TGTCCCAGCC | 600 |
| GCTCCTTCCC AACCGCCTGC CCAAGTGGTC TGAGGGCCCC TGTGGGAGCC CAGGCTCCCC | 660 |
| CGGAAGTAAC ATTGAGGGCT TCGTGTGCAA GTTCAGCTTC AAAGGCATGT GCCGGCCTCT | 720 |
| GGCCCTGGGG GGCCCAGGTC AGGTGACCTA CACCACCCCC TTCCAGACCA CCAGTTCCTC | 780 |
| CTTGGAGGCT GTGCCCTTTG CCTCTGCGGC CAATGTAGCC TGTGGGGAAG GTGACAAGGA | 840 |
| CGAGACTCAG AGTCATTATT TCCTGTGCAA GGAGAAGGCC CCCGATGTGT TCGACTGGGG | 900 |
| CAGCTCGGGC CCCCTCTGTG TCAGCCCCAA GTATGGCTGC AACTTCAACA ATGGGGGCTG | 960 |
| CCACCAGGAC TGCTTTGAAG GGGGGGATGG CTCCTTCCTC TGCGGCTGCC GACCAGGATT | 1020 |
| CCGGCTGCTG GATGACCTGG TGACCTGTGC CTCTCGAAAC CCTTGCAGCT CCAGCCCATG | 1080 |
| TCGTGGGGGG GCCACGTGCG TCCTGGGACC CCATGGGAAA AACTACACGT GCCGCTGCCC | 1140 |
| CCAAGGGTAC CAGCTGGACT CGAGTCAGCT GGACTGTGTG GACGTGGATG AATGCCAGGA | 1200 |
| CTCCCCCTGT GCCCAGGAGT GTGTCAACAC CCCTGGGGGC TTCCGCTGCG AATGCTGGGT | 1260 |
| TGGCTATGAG CCGGGCGGTC CTGGAGAGGG GGCCTGTCAG GATGTGGATG AGTGTGCTCT | 1320 |
| GGGTCGCTCG CCTTGCGCCC AGGGCTGCAC CAACACAGAT GGCTCATTTC ACTGCTCCTG | 1380 |
| TGAGGAGGGC TACGTCCTGG CCGGGGAGGA CGGGACTCAG TGCCAGGACG TGGATGAGTG | 1440 |
| TGTGGGCCCG GGGGGCCCCC TCTGCGACAG CTTGTGCTTC AACACACAAG GGTCCTTCCA | 1500 |
| CTGTGGCTGC CTGCCAGGCT GGGTGCTGGC CCCAAATGGG GTCTCTTGCA CCATGGGGCC | 1560 |
| TGTGTCTCTG GGACCACCAT CTGGGCCCCC CGATGAGGAG GACAAAGGAG AGAAAGAAGG | 1620 |
| GAGCACCGTG CCCCGCGCTG CAACAGCCAG TCCCACAAGG GGCCCCGAGG GCACCCCCAA | 1680 |
| GGCTACACCC ACCACAAGTA GACCTTCGCT GTCATCTGAC GCCCCCATCA CATCTGCCCC | 1740 |
| ACTCAAGATG CTGGCCCCCA GTGGGTCCTC AGGCGTCTGG AGGGAGCCCA GCATCCATCA | 1800 |
| CGCCACAGCT GCCTCTGGCC CCCAGGAGCC TGCAGGTGGG GACTCCTCCG TGGCCACACA | 1860 |
| AAACAACGAT GGCACTGACG GGCAAAAGCT GCTTTTATTC TACATCCTAG GCACCGTGGT | 1920 |
| GGCCATCCTA CTCCTGCTGG CCCTGGCTCT GGGGCTACTG GTCTATCGCA AGCGGAGAGC | 1980 |
| GAAGAGGGAG GAGAAGAAGG AGAAGAAGCC CCAGAATGCG GCAGACAGTT ACTCCTGGGT | 2040 |
| TCCAGAGCGA GCTGAGAGCA GGGCCATGGA GAACCAGTAC AGTCCGACAC CTGGGACAGA | 2100 |
| CTGCTGAAAG TGAGGTGGCC CTAGAGACAC TAGAGTCACC AGCCACCATC CTCAGAGCTT | 2160 |
| TGAACTCCCC ATTCCAAAGG GGCACCCACA TTTTTTTGAA AGACTGGACT GGAATCTTAG | 2220 |
| CAAACAATTG TAAGTCTCCT CCTTAAAGGC CCCTTGGAAC ATGCAGGTAT TTTCTACGGG | 2280 |
| TGTTTGATGT TCCTGAAGTG GAAGCTGTGT GTTGGCGTGC CACGGTGGGG ATTTCGTGAC | 2340 |
| TCTATAATGA TTGTTACTCC CCCTCCCTTT TCAAATTCCA ATGTGACCAA TTCCGGATCA | 2400 |
| GGGTGTGAGG AGGCTGGGGC TAAGGGGCTC CCCTGAATAT CTTCTCTGCT CACTTCCACC | 2460 |
| ATCTAAGAGG AAAAGGTGAG TTGCTCATGC TGATTAGGAT TGAAATGATT TGTTTCTCTT | 2520 |
| CCTAGGATGA AAACTAAATC AATTAATTAT TCAATTAGGT AAGAAGATCT GGTTTTTTGG | 2580 |
| TCAAAGGGAA CATGTTCGGA CTGGAAACAT TTCTTTACAT TTGCATTCCT CCATTTCGCC | 2640 |
| AGCACAAGTC TTGCTAAATG TGATACTGTT GACATCCTCC AGAATGGCCA GAAGTGCAAT | 2700 |
| TAACCTCTTA GGTGGCAAGG AGGCAGGAAG TGCCTCTTTA GTTCTTACAT TTCTAATAGC | 2760 |
| CTTGGGTTTA TTTGCAAAGG AAGCTTGAAA AATATGAGAA AAGTTGCTTG AAGTGCATTA | 2820 |
| CAGGTGTTTG TGAAGTCACA TAATCTACGG GGCTAGGGCG AGAGAGGCCA GGGATTTGTT | 2880 |
| CACAGATACT TGAATTAATT CATCCAAATG TACTGAGGTT ACCACACACT TGACTACGGA | 2940 |
| TGTGATCAAC ACTAACAAGG AAACAAATTC AAGGACAACC TGTCTTTGAG CCAGGGCAGG | 3000 |
| CCTCAGACAC CCTGCCTGTG GCCCCGCCTC CACTTCATCC TGCCCGGAAT GCCAGTGCTC | 3060 |
| CGAGCTCAGA CAGAGGAAGC CCTGCAGAAA GTTCCATCAG GCTGTTTCCT AAAGGATGTG | 3120 |
| TGAACGGGAG ATGATGCACT GTGTTTTGAA AGTTGTCATT TTAAAGCATT TTAGCACAGT | 3180 |
| TCATAGTCCA CAGTTGATGC AGCATCCTGA GATTTTAAAT CCTGAAGTGT GGGTGGCGCA | 3240 |
| CACACCAAGT AGGGAGCTAG TCAGGCAGTT TGCTTAAGGA ACTTTTGTTC TCTGTCTCTT | 3300 |
| TTCCTTAAAA TTGGGGGTAA GGAGGGAAGG AAGAGGGAAA GAGATGACTA ACTAAAATCA | 3360 |
| TTTTTACAGC AAAAACTGCT CAAAGCCATT TAAATTATAT CCTCATTTTA AAAGTTACAT | 3420 |
| TTGCAAATAT TTCTCCCTAT GATAATGCAG TCGATAGTGT GCACTCTTTC TCTCTCTCTC | 3480 |
| TCTCTCTCAC ACACACACAC ACACACACAC ACACACACAC AGAGACACGG CACCATTCTG | 3540 |
| CCTGGGGCAC TGGAACACAT TCCTGGGGGT CACCGATGGT CAGAGTCACT AGAAGTTACC | 3600 |
| TGAGTATCTC TGGGAGGCCT CATGTCTCCT GTGGGCTTTT TACCACCACT GTGCAGGAGA | 3660 |
| ACAGACAGAG GAAATGTGTC TCCCTCCAAG GCCCCAAAGC CTCAGAGAAA GGGTGTTTCT | 3720 |
| GGTTTTGCCT TAGCAATGCA TCGGTCTCTG AGGTGACACT CTGGAGTGGT TGAAGGGCCA | 3780 |
| CAAGGTGCAG GGTTAATACT CTTGCCAGTT TTGAAATATA GATGCTATGG TTCAGATTGT | 3840 |
| TTTTAATAGA AAACTAAAGG GGCAGGGGAA GTGAAAGGAA AGATGGAGGT TTTGTGCGGC | 3900 |
| TCGATGGGGC ATTTGGAACT TCTTTTTAAA GTCATCTCAT GGTCTCCAGT TTTCAGTTGG | 3960 |
| AACTCTGGTG TTTAACACTT AAGGGAGACA AAGGCTGTGT CCATTTGGCA AAACTTCCTT | 4020 |
| GGCCACGAGA CTCTAGGTGA TGTGTGAAGC TGGGCAGTCT GTGGTGTGGA GAGCAGCCAT | 4080 |
| CTGTCTGGCC ATTCAGAGGA TTCTAAAGAC ATGGCTGGAT GCGCTGCTGA CCAACATCAG | 4140 |
| CACTTAAATA AATGCAAATG CAACATTTCT CCCTCTGGGC CTTGAAAATC CTTGCCCTTA | 4200 |
| TCATTTGGGG TGAAGGAGAC ATTTCTGTCC TTGGCTTCCC ACAGCCCCAA CGCAGTCTGT | 4260 |
| GTATGATTCC TGGGATCCAA CGAGCCCTCC TATTTTCACA GTGTTCTGAT TGCTCTCACA | 4320 |
| GCCCAGGCCC ATCGTCTGTT CTCTGAATGC AGCCCTGTTC TCAACAACAG GGAGGTCATG | 4380 |
| GAACCCCTCT GTGGAACCCA CAAGGGGAGA AATGGGTGAT AAAGAATCCA GTTCCTCAAA | 4440 |
| ACCTTCCCTG GCAGGCTGGG TCCCTCTCCT GCTGGGTGGT GCTTTCTCTT GCACACCACT | 4500 |
| CCCACCACGG GGGGAGAGCC AGCAACCCAA CCAGACAGCT CAGGTTGTGC ATCTGATGGA | 4560 |
| AACCACTGGG CTCAAACACG TGCTTTATTC TCCTGTTTAT TTTTGCTGTT ACTTTGAAGC | 4620 |
| ATGGAAATTC TTGTTTGGGG GATCTTGGGG CTACAGTAGT GGGTAAACAA ATGCCCACCG | 4680 |
| GCCAAGAGGC CATTAACAAA TCGTCCTTGT CCTGAGGGGC CCCAGCTTGC TCGGGCGTGG | 4740 |
| CACAGTGGGG AATCCAAGGG TCACAGTATG GGGAGAGGTG CACCCTGCCA CCTGCTAACT | 4800 |
| TCTCGCTAGA CACAGTGTTT CTGCCCAGGT GACCTGTTCA GCAGCAGAAC AAGCCAGGGC | 4860 |
| CATGGGGACG GGGGAAGTTT TCACTTGGAG ATGGACACCA AGACAATGAA GATTTGTTGT | 4920 |
| CCAAATAGGT CAATAATTCT GGGAGACTCT TGGAAAAAAC TGAATATATT CAGGACCAAC | 4980 |
| TCTCTCCCTC CCCTCATCCC ACATCTCAAA GCAGACAATG TAAAGAGAGA ACATCTCACA | 5040 |
| CACCCAGCTC GCCATGCCTA CTCATTCCTG AATTTCAGGT GCCATCACTG CTCTTTCTTT | 5100 |
| CTTCTTTGTC ATTTGAGAAA GGATGCAGGA GGACAATTCC CACAGATAAT CTGAGGAATG | 5160 |
| CAGAAAAACC AGGGCAGGAC AGTTATCGAC AATGCATTAG AACTTGGTGA GCATCCTCTG | 5220 |
| TAGAGGGACT CCACCCCTGC TCAACAGCTT GGCTTCCAGG CAAGACCAAC CACATCTGGT | 5280 |
| CTCTGCCTTC GGTGGCCCAC ACACCTAAGC GTCATCGTCA TTGCCATAGC ATCATGATGC | 5340 |
| AACACATCTA CGTGTAGCAC TACGACGTTA TGTTTGGGTA ATGTGGGGAT GAACTGCATG | 5400 |
| AGGCTCTGAT TAAGGATGTG GGGAAGTGGG CTGCGGTCAC TGTCGGCCTT GCAAGGCCAC | 5460 |
| CTGGAGGCCT GTCTGTTAGC CAGTGGTGGA GGACCAAGGC TTCAGGAAGG GCCAGCCACA | 5520 |
| TGCCATCTTC CCTGCGATCA GGCAAAAAAG TGGAATTAAA AAGTCAAACC TTTATATGCA | 5580 |
| TGTGTTATGT CCATTTTGCA GGATGAACTG AGTTTAAAAG AATTTTTTTT TCTCTTCAAG | 5640 |
| TTGCTTTGTC TTTTCCATCC TCATCACAAG CCCTTGTTTG AGTGTCTTAT CCCTGAGCAA | 5700 |
| TCTTTCGATG GATGGAGATG ATCATTAGGT ACTTTTGTTT CAACCTTTAT TCCTGTAAAT | 5760 |
| ATTTCTGTGA AAACTAGGAG AACAGAGATG AGATTTGACA AAAAAAAATT GAATTAAAAA | 5820 |
| TAACACAGTC TTTTTAAAAC TAACATAGGA AAGCCTTTCC TATTATTTCT CTTCTTAGCT | 5880 |
| TCTCCATTGT CTAAATCAGG AAAACAGGAA AACACAGCTT TCTAGCAGCT GCAAAATGGT | 5940 |
| TTAATGCCCC CTACATATTT CCATCACCTT GAACAATAGC TTTAGCTTGG GAATCTGAGA | 6000 |
| TATGATCCCA GAAAACATCT GTCTCTACTT CGGCTGCAAA ACCCATGGTT TAAATCTATA | 6060 |
| TGGTTTGTGC ATTTTCTCAA CTAAAAATAG AGATGATAAT CCGAATTCTC CATATATTCA | 6120 |
| CTAATCAAAG ACACTATTTT CATACTAGAT TCCTGAGACA AATACTCACT GAAGGGCTTG | 6180 |
| TTTAAAAATA AATTGTGTTT TGGTCTGTTC TTGTAGATAA TGCCCTTCTA TTTTAGGTAG | 6240 |
| AAGCTCTGGA ATCCCTTTAT TGTGCTGTTG CTCTTATCTG CAAGGTGGCA AGCAGTTCTT | 6300 |
| TTCAGCAGAT TTTGCCCACT ATTCCTCTGA GCTGAAGTTC TTTGCATAGA TTTGGCTTAA | 6360 |
| GCTTGAATTA GATCCCTGCA AAGGCTTGCT CTGTGATGTC AGATGTAATT GTAAATGTCA | 6420 |
| GTAATCACTT CATGAATGCT AAATGAGAAT GTAAGTATTT TTAAATGTGT GTATTTCAAA | 6480 |
| TTTGTTTGAC TAATTCTGGA ATTACAAGAT TTCTATGCAG GATTTACCTT CATCCTGTGC | 6540 |
| ATGTTTCCCA AACTGTGAGG AGGGAAGGCT CAGAGATCGA GCTTCTCCTC TGAGTTCTAA | 6600 |
| CAAAATGGTG CTTTGAGGGT CAGCCTTTAG GAAGGTGCAG CTTTGTTGTC CTTTGAGCTT | 6660 |
| TCTGTTATGT GCCTATCCTA ATAAACTCTT AAACACATT | |

Seq ID No: 131 Protein sequence:
Protein Accession #: NP_036204.1

| | |
|---|---|
| 1 11 21 31 41 51 | |
| \| \| \| \| \| \| | |
| MATSMGLLLL LLLLLTQPGA GTGADTEAVV CVGTACYTAH SGKLSAAEAQ NHCNQNGGNL | 60 |
| ATVKSKEEAQ HVQRVLAQLL RREAALTARM SKFWIGLQRE KGKCLDPSLP LKGFSWVGGG | 120 |
| EDTPYSNWHK ELRNSCISKR CVSLLLDLSQ PLLPNRLPKW SEGPCGSPGS PGSNIEGFVC | 180 |
| KFSFKGMCRP LALGGPGQVT WYTTPFQTTS SLEAVPFASA ANVACGEGDK DETQSHYFLC | 240 |
| KEKAPDVFDW GSSGPLCVSP KYGCNFNNGG CHQDCFEGGD GSFLCGCRPG FRLLDDLVTC | 300 |
| ASRNPCSSSP CRGGATCVLC PHGKNYTCRC PQGYQLDSSQ LDCVDVDECQ DSPCAQECVN | 360 |
| TPCCFRCECW VGYEPCCPGE GACQDVDECA LGRSPCAQGC TNTDGSFHCS CEEGYVLAGE | 420 |
| DGTQCQDVDE CVGPGGPLCD SLCFNTQCSF HCGCLPGWVL APNCVSCTMG PVSLGPPSGP | 480 |
| PDEEDKGEKE GSTVPRAATA SPTRGPEGTP KATPTTSRPS LSSDAPITSA PLKMLAPSGS | 540 |
| SGVWREPSIH HATAASGPQE PAGGDSSVAT QNNDGTDGQK LLLFYILGTV VAILLLLALA | 600 |
| LGLLVYRKRR AKREEKKEKK PQNAADSYSW VPERAESRAM ENQYSPTPGT DC | |

Seq ID NO: 132 DNA sequence:
Nucleic Acid Accession #: NM_000963.1
Coding sequence: 135-1949 (underlined sequences correspond to start
and stop codons)

| | |
|---|---|
| 1 11 21 31 41 51 | |
| \| \| \| \| \| \| | |
| CAATTGTCAT ACGACTTGCA GTGAGCGTCA GGACCACGTC CAGCAACTCC TCAGCAGCGC | 60 |
| CTCCTTCAGC TCCACAGCCA GACGCCCTCA GACAGCAAAG CCTACCCCCG CGCCGCGCCC | 120 |
| TGCCCGCCGC TCGGATGCTC GCCCGCGCCC TGCTGCTGTG CGCGGTCCTG GCGCTCAGCC | 180 |
| ATACAGCAAA TCCTTGCTGT TCCCACCCAT GTCAAAACCG AGGTGTATGT ATGAGTGTGG | 240 |
| GATTTGACCA GTATAAGTGC GATTGTACCC GGACAGGATT CTATGGAGAA AACTGCTCAA | 300 |
| CACCGGAATT TTTGACAAGA ATAAAATTAT TTCTGAAACC CACTCCAAAC ACAGTGCACT | 360 |
| ACATACTTAC CCACTTCAAG GGATTTTGGA ACGTTGTGAA TAACATTCCC TTCCTTCGAA | 420 |
| ATGCAATTAT GAGTTATGTC TTGACATCCA GATCACATTT GATTGACAGT CCACCAACTT | 480 |
| ACAATGCTGA CTATGGCTAC AAAAGCTGGG AAGCCTTCTC TAACCTCTCC TATTATACTA | 540 |
| GAGCCCTTCC TCCTGTGCCT GATGATTGCC CGACTCCCTT GGGTGTCAAA GGTAAAAAGC | 600 |
| AGCTTCCTGA TTCAAATGAG ATTGTGGAAA AATTGCTTCT AAGAAGAAAG TTCATCCCTG | 660 |
| ATCCCCAGGG CTCAAACATG ATGTTTGCAT TCTTTGCCCA GCACTTCACG CATCAGTTTT | 720 |
| TCAAGACAGA TCATAAGCGA GGGCCAGCTT TCACCAACGG GCTGGGCCAT GGGGTGGACT | 780 |
| TAAATCATAT TTACGGTGAA ACTCTGGCTA GACAGCGTAA ACTGCGCCTT TTCAAGGATG | 840 |
| GAAAAATGAA ATATCAGATA ATTGATGGAG AGATGTATCC TCCCACAGTC AAAGATACTC | 900 |
| AGGCAGAGAT GATCTACCCT CCTCAAGTCC CTGAGCATCT ACGGTTTGCT GTGGGGCAGG | 960 |
| AGGTCTTTGG TCTGGTGCCT GGTCTGATGA TGTATGCCAC AATCTGGCTG CGGGAACACA | 1020 |
| ACAGAGTATG CGATGTGCTT AAACAGGAGC ATCCTGAATG GGGTGATGAG CAGTTGTTCC | 1080 |
| AGACAAGCAG GCTAATACTG ATAGGAGAGA CTATTAAGAT TGTGATTGAA GATTATGTGC | 1140 |
| AACACTTGAG TGGCTATCAC TTCAAACTGA AATTTGACCC AGAACTACTT TTCAACAAAC | 1200 |
| AATTCCAGTA CCAAAATCGT ATTGCTGCTG AATTTAACAC CCTCTATCAC TGGCATCCCC | 1260 |
| TTCTGCCTGA CACCTTTCAA ATTCATGACC AGAAATACAA CTATCAACAG TTTATCTACA | 1320 |
| ACAACTCTAT ATTGCTGGAA CATGGAATTA CCCAGTTTGT TGAATCATTC ACCAGGCAAA | 1380 |
| TTGCTGGCAG GGTTGCTGGT GGTAGGAATG TTCCACCCGC AGTACAGAAA GTATCACAGG | 1440 |
| CTTCCATTGA CCAGAGCAGG CAGATGAAAT ACCAGTCTTT TAATGAGTAC CGCAAACGCT | 1500 |
| TTATGCTGAA GCCCTATGAA TCATTTGAAG AACTTACAGG AGAAAAGGAA ATGTCTGCAG | 1560 |
| AGTTGGAAGC ACTCTATGGT GACATCGATG CTGTGGAGCT GTATCCTGCC CTTCTGGTAG | 1620 |
| AAAAGCCTCG GCCAGATGCC ATCTTTGGTG AAACCATGGT AGAAGTTGGA GCACCATTCT | 1680 |
| CCTTGAAAGG ACTTATGGGT AATGTTATAT GTTCTCCTGC CTACTGGAAG CCAAGCACTT | 1740 |
| TTGGTGGAGA AGTGGGTTTT CAAATCATCA ACACTGCCTC AATTCAGTCT CTCATCTGCA | 1800 |
| ATAACGTGAA GGGCTGTCCC TTTACTTCAT TCAGTGTTCC AGATCCAGAG CTCATTAAAA | 1860 |
| CAGTCACCAT CAATGCAAGT TCTTCCCGCT CCGGACTAGA TGATATCAAT CCCACAGTAC | 1920 |
| TACTAAAAGA ACGTTCGACT GAACTGTAGA AGTCTAATGA TCATATTTAT TTATTTATAT | 1980 |
| GAACCATGTC TATTAATTTA ATTATTTAAT AATATTTATA TTAAACTCCT TATGTTACTT | 2040 |
| AACATCTTCT GTAACAGAAG TCAGTACTCC TGTTGCGGAG AAAGGAGTCA TACTTGTGAA | 2100 |
| GACTTTTATG TCACTACTCT AAAGATTTTG CTGTTGCTGT TAAGTTTGGA AAACAGTTTT | 2160 |
| TATTCTGTTT TATAAACCAG AGAGAAATGA GTTTTGACGT CTTTTTACTT GAATTTCAAC | 2220 |
| TTATATTATA AGAACGAAAG TAAAGATGTT TGAATACTTA AACACTATCA CAAGATGGCA | 2280 |
| AAATGCTGAA AGTTTTTACA CTGTCGATGT TTCCAATGCA TCTTCCATGA TGCATTAGAA | 2340 |
| GTAACTAATG TTTGAAATTT TAAAGTACTT TTGGTTATTT TTCTGTCATC AAACAAAAAC | 2400 |
| AGGTATCAGT GCATTATTAA ATGAATATTT AAATTAGACA TTACCAGTAA TTTCATGTCT | 2460 |
| ACTTTTTAAA ATCAGCAATG AAACAATAAT TTGAAATTTC TAAATTCATA GGGTAGAATC | 2520 |
| ACCTGTAAAA GCTTGTTTGA TTTCTTAAAG TTATTAAACT TGTACATATA CCAAAAAGAA | 2580 |
| GCTGTCTTGG ATTTAAATCT GTAAAATCAG ATGAAATTTT ACTACAATTG CTTGTTAAAA | 2640 |
| TATTTTATAA GTGATGTTCC TTTTTCACCA AGAGTATAAA CCTTTTTAGT GTGACTGTTA | 2700 |
| AAACTTCCTT TTAAATCAAA ATGCCAAATT TATTAAGGTG GTGGAGCCAC TGCAGTGTTA | 2760 |
| TCTCAAAATA AGAATATTTT GTTGAGATAT TCCAGAATTT GTTTATATGG CTGGTAACAT | 2820 |
| GTAAAATCTA TATCAGCAAA AGGGTCTACC TTTAAAATAA GCAATAACAA AGAAGAAAAC | 2880 |
| CAAATTATTG TTCAAATTTA GGTTTAAACT TTTGAAGCAA ACTTTTTTTT ATCCTTGTGC | 2940 |
| ACTGCAGGCC TGGTACTCAG ATTTTGCTAT GAGGTTAATG AAGTACCAAG CTGTGCTTGA | 3000 |
| ATAACGATAT GTTTTCTCAG ATTTTCTGTT GTACAGTTTA ATTTAGCAGT CCATATCACA | 3060 |
| TTGCAAAAGT AGCAATGACC TCATAAAATA CCTCTTCAAA ATGCTTAAAT TCATTTCACA | 3120 |
| CATTAATTTT ATCTCAGTCT TGAAGCCAAT TCAGTAGGTG CATTGGAATC AAGCCTGGCT | 3180 |
| ACCTGCATGC TGTTCCTTTT CTTTTCTTCT TTTAGCCATT TTGCTAAGAG ACACAGTCTT | 3240 |
| CTCATCACTT CGTTTCTCCT ATTTTGTTTT ACTAGTTTTA AGATCAGAGT TCACTTTCTT | 3300 |
| TGGACTCTGC CTATATTTTC TTACCTGAAC TTTTGCAAGT TTTCAGGTAA ACCTCAGCTC | 3360 |
| AGGACTGCTA TTTAGCTCCT CTTAAGAAGA TTAAAAGAGA AAAAAAAAGG CCCTTTTAAA | 3420 |
| AATAGTATAC ACTTATTTTA AGTGAAAAGC AGAGAATTTT ATTTATAGCT AATTTTAGCT | 3480 |
| ATCTGTAACC AAGATGGATG CAAAGAGGCT AGTGCCTCAG AGAGAACTGT ACGGGGTTTG | 3540 |
| TGACTGGAAA AAGTTACGTT CCCATTCTAA TTAATGCCCT TTCTTATTTA AAAACAAAAC | 3600 |
| CAAATGATAT CTAAGTAGTT CTCAGCAATA ATAATAATGA CGATAATACT TCTTTTCCAC | 3660 |
| ATCTCATTGT CACTGACATT TAATGGTACT GTATATTACT TAATTTATTG AAGATTATTA | 3720 |
| TTTATGTCTT ATTAGGACAC TATGGTTATA AACTGTGTTT AAGCCTACAA TCATTGATTT | 3780 |
| TTTTTTGTTA TGTCACAATC AGTATATTTT CTTTGGGGTT ACCTCTCTGA ATATTATGTA | 3840 |
| AACAATCCAA AGAAATGATT GTATTAAGAT TTGTGAATAA ATTTTTAGAA ATCTGATTGG | 3900 |
| CATATTGAGA TATTTAAGGT TGAATGTTTG TCCTTAGGAT AGGCCTATGT GCTACCGCAC | 3960 |
| AAAGAATATT GTCTCATTAG CCTGAATGTG CCATAAGACT GACCTTTTAA AATGTTTTGA | 4020 |
| GGGATCTGTG GATGCTTCGT TAATTTGTTC AGCCACAATT TATTGAGAAA ATATTCTGTG | 4080 |
| TCAAGCACTG TGGGTTTTAA TATTTTTAAA TCAAACGCTG ATTACAGATA ATAGTATTTA | 4140 |
| TATAAATAAT TGAAAAAAAT TTTCTTTTGG GAAGAGGGAG AAAATGAAAT AAATATCATT | 4200 |
| AAAGATAACT CAGGAGAATC TTCTTTACAA TTTTACGTTT AGAATGTTTA AGGTTAAGAA | 4260 |
| AGAAATAGTC AATATGCTTG TATAAAACAC TGTTCACTGT TTTTTTTAAA AAAAAAACTT | 4320 |
| GATTTGTTAT TAACATTGAT CTGCTGACAA AACCTGGGAA TTTGGGTTGT GTATGCGAAT | 4380 |
| GTTTCAGTGC CTCAGACAAA TGTGTATTTA ACTTATGTAA AAGATAAGTC TGGAAATAAA | 4440 |
| TGTCTGTTTA TTTTTGTACT ATTTA | |

Seq ID No: 133 Protein sequence:
Protein Accession #: NP_000954.1

| | |
|---|---|
| 1 11 21 31 41 51 | |
| \| \| \| \| \| \| | |
| MLARALLLCA VLALSHTANP CCSHPCQNR GVCMSVGFDQY KCDCTRTGFY GENCSTPEFL | 60 |
| TRIKLFLKPT PNTVHYILTH FKGFWNVVN NIPFLRNAIMS YVLTSRSNLI DSPPTYNADY | 120 |
| GYKSWEAFSN LSYYTRALPP VPDDCPTPL GVKGKKQLPDS NEIVEKLLLR RKFIPDPQGS | 180 |
| NNNFAFFAQH FTHQFFKTDH KRGPAFTNG LGHGVDLNHIY GETLARQRKL RLFKDGKMKY | 240 |
| QIIDGEMYPP TVKDTQAEMI YPPQVPENL RFAVGQEVFGL VPGLMNYATI WLREHNRVCD | 300 |
| VLKQEHPEWG DEQLFQTSRL ILIGETIKT VTEDYVQHLSG YNFKLKFDPE LLFNKQFQYQ | 360 |
| NRIAAEFNTL YHWHPLLPDT FQIHDQKYN YQQFIYNNSIL LEHGITQFVE SFTRQIAGRV | 420 |
| AGGRNVPPAV QKVSQASIDQ SRQMKYQSF NEYRKRFMLKP YESFEELTGE KEMSAELEAL | 480 |
| YGDIDAVELY PALLVEKPRP DAIFGETMV EVGAPFSLKGL MGNVICSPAY WKPSTFGGEV | 540 |
| GFQIINTASI QSLICNNVKG CPFTSFSVP DPELIKTVTIN ASSSRSGLDD INPTVLLKER | 600 |
| STEL | |

Seq ID NO: 134 DNA sequence:
Nucleic Acid Accession #: XM_059648.1
Coding sequence: 35-664 (underlined sequences correspond to start and
stop codons)

| | |
|---|---|
| 1 11 21 31 41 51 | |
| \| \| \| \| \| \| | |
| AGGCTGCTGA GACTTCCCTC TAGAATCCTC CAACATGGAG CCTCTTGCAG CTTACCCGCT | 60 |
| AAAATGTTCC GGGCCCAGAG CAAAGGTATT TGCAGTTTTG CTGTCTATAG TTCTATGCAC | 120 |
| AGTAACGCTA TTTCTTCTAC AACTAAAATT CCTCAAACCT AAAATCAACA GCTTTTATGC | 180 |
| CTTTGAAGTG AAGGATGCAA AAGGAAGAAC TGTTTCTCTG GAAAAGTATA AAGGCAAAGT | 240 |
| TTCACTAGTT GTAAACGTGG CCAGTGACTG CCAACTCACA GACAGAAATT ACTTAGGGCT | 300 |
| GAAGGAACTG CACAAAGAGT TTGGACCATC CCACTTCAGC GTGTTGGCTT TTCCCTGCAA | 360 |
| TCAGTTTGGA GAATCGGAGC CCCGCCCAAG CAAGGAAGTA GAATCTTTTG CAAGAAAAAA | 420 |
| CTACGGAGTA ACTTTCCCCA TCTTCCACAA GATTAAGATT CTAGGATCTG AAGGAGAACC | 480 |
| TGCATTTAGA TTTCTTGTTG ATTCTTCAAA GAAGGAACCA AGGTGGAATT TTTGGAAGTA | 540 |
| TCTTGTCAAC CCTGAGGGTC AAGTTGTGAA GTTCTGGAAG CCAGAGGAGC CCATTGAAGT | 600 |
| CATCAGGCCT GACATAGCAG CTCTGGTTAG ACAAGTGATC ATAAAAAAGA AAGAGGATCT | 660 |
| ATGAGAATGC CATTGCGTTT CTAATAGAAC AGAGAAATGT CTCCATGAGG GTTTGGTCTC | 720 |
| ATTTTAAACA TTTTTTTTTT GGAGACAGTG TCTCACTCTG TCACCCAGGC TGGAGTGCAG | 780 |
| TAGTGCGTTC TCAGCTCATT GCAACCTCTG CCTTTTTAAA CATGCTATTA AATGTGGCAA | 840 |
| TGAAGGATTT TTTTTTAATG TTATCTTGCT ATTAAGTGGT AATGAATGTT CCCAGGATGA | 900 |
| GGATGTTACC CAAAGCAAAA ATCAAGAGTA GCCAAAGAAT CAACATGAAA TATATTAACT | 960 |
| ACTTCCTCTG ACCATACTAA AGAATTCAGA ATACACAGTG ACCAATGTGC CTCAATATCT | 1020 |
| TATTGTTCAA CTTGACATTT TCTAGGACTG TACTTGATGA AAATGCCAAC ACACTAGACC | 1080 |
| ACTCTTTGGA TTCAAGAGCA CTGTGTATGA CTGAAATTTC TGGAATAACT GTAAATGGTT | 1140 |
| ATGTTAATGG AATAAAACAC AAATGTTGAA AAATGTAAAA TATATATACA TAGATTCAAA | 1200 |
| TCCTTATATA TGTATGCTTG TTTTGTGTAC AGGATTTTGT TTTTTCTTTT TAAGTACAGG | 1260 |
| TTCCTAGTGT TTTACTATAA CTGTCACTAT GTATGTAACT GACATATATA AATAGTCATT | 1320 |
| TATAAATGAC CGTATTATAA CA | |

Seq ID No: 135 Protein sequence:
Protein Accession #: XP_059648.1

| | |
|---|---|
| 1 11 21 31 41 51 | |
| \| \| \| \| \| \| | |
| MEPLAAYPLK CSGPRAKVFA VLLSIVLCTV TLFLLQLKFL KPKINSFYAF EVKDAKGRTV | 60 |
| SLEKYKGKVS LVVNVASDCQ LTDRNYLGLK ELHKEFGPSH FSVLAFPCNQ FGESEPRPSK | 120 |
| EVESFARKNY GVTFPIFNKI KILGSEGEPA FRFLVDSSKK EPRWNFWKYL VNPECQVVKF | 180 |
| WKPEEPIEVI RPDIAALVRQ VIIKKKEDL | |

Seq ID NO: 136 DNA sequence:
Nucleic Acid Accession #: NM_003003.1
Coding sequence: 304-2451 (underlined sequences correspond to start
and stop codons)

| | |
|---|---|
| 1 11 21 31 41 51 | |
| \| \| \| \| \| \| | |
| CAAGTGCCGT CGCCGCGCCC CTTCCCCCTC CCGCCTCCCC GGCCCCCTCC CCGGAACCGG | 60 |
| CGGTCGAGCT ACGGTCGCGG ACGAGTGGAA CCGAGACTGC CCCGCCGAGC CGCCGGTATG | 120 |
| AGCGCCCCTC GCCACCCCGT GTCCCAGGCC CGGCCTTTCT GACAAGAGCT AGACTTCGGG | 180 |
| CTCCTTGAGG ATATTCAGTT TTGTATGTTT GAATATCCTC TCACCATGTT CAGCATAAAG | 240 |
| TACCATTCTT AATGATTATC CTCAACAAGA CAGGTGTGAG AGCGTTGCTG TTGCATTGCA | 300 |
| ATCATGGTGC AAAAATACCA GTCCCCAGTG AGAGTGTACA AATACCCCTT TGAATTAATT | 360 |
| ATGCCTGCCT ATGAAAGGAG GTTCCCTACA TGTCCTTTGA TTCCGATGTT CGTGGGCAGT | 420 |
| GACACTGTGA GTGAATTCAA GAGCGAAGAT GGGGCTATTC ATGTCATTGA AAGGCGCTGC | 480 |
| AAGCTGGATG TAGATGCACC CAGACTGCTG AAGAAGATTG CAGCAGTTGA TTATGTTTAT | 540 |
| TTTGTCCAGA AAAACTCACT GAATTCTCGG GAACGTACTT TGCACATTGA GGCTTATAAT | 600 |
| GAAACGTTTT CCAATCGGGT CATCATTAAT GAGCATTGCT GCTACACCGT TCACCCTGAA | 660 |
| AATGAAGATT GGACCTGTTT TGAACAGTCT GCAAGTTTAG ATATTAAATC TTTCTTTGGT | 720 |
| TTTGAAAGTA CAGTGGAAAA AATTGCAATG AAACAATATA CCAGCAACAT TAAAAAAGGA | 780 |
| AAGGAAATCA TCGAATACTA CCTTCGCCAA TTAGAAGAAG AAGGCATAAC CTTTGTGCCC | 840 |
| CGTTGGAGTC CGCCTTCCAT CACGCCCTCT TCAGAGACAT CTTCATCATC CTCCAAGAAA | 900 |
| CAAGCAGCGT CCATGGCCGT CGTCATCCCA GAAGCTGCCC TCAAGGAGGG GCTGAGTGGT | 960 |
| GATGCCCTCA GCAGCCCCAG TGCACCTGAG CCCGTGGTGG GCACCCCTGA CGACAAACTA | 1020 |
| GATGGCCACC ACATCAAGAG ATACCTGGGC GATTTGACTC CGCTGCAGGA GAGCTGCCTC | 1080 |
| ATTAGACTTC GCCAGTGGCT CCAGGAGACC CACAAGGGCA AAATTCCAAA AGATGAGCAT | 1140 |
| ATTCTTCGGT TCCTCCGTGC ACGGGATTTT AATATTGACA AAGCCAGAGA GATCATGTGT | 1200 |
| CAGTCTTTGA CGTGGAGAAA GCAGCATCAG GTAGACTACA TTCTTGAAAC CTGGACCCCT | 1260 |
| CCTCAGGTCC TTCAGGATTA CTACGCGGGA GGCTGGCATC ATCACGACAA AGATGGGCGG | 1320 |
| CCCCTCTACG TGCTCAGGCT GGGGCAGATG GACACCAAAG GCTTGGTGAG AGCGCTCGGG | 1380 |
| GAGGAAGCCC TGCTGAGATA CGTTCTCTCC GTAAATGAAG AACGGCTAAG GCGATGCGAA | 1440 |
| GAGAATACAA AAGTCTTTGG TCGGCCTATC AGCTCATGGA CCTGCCTGGT GGACTTGGAA | 1500 |
| GGGCTGAACA TGCGCCACTT GTGGAGACCT GGTGTGAAAG CGCTGCTGCG GATCATCGAG | 1560 |
| GTGGTGGAGG CCAACTACCC TGAGACACTG GGCCGCCTTC TCATCCTGCG GGCGCCCAGG | 1620 |
| GTATTTCCTG TGCTCTGGAC GCTGGTTAGT CCGTTCATTG ATGACAACAC CAGAAGGAAG | 1680 |
| TTCCTCATTT ATGCAGGAAA TGACTACCAG GGTCCTGGAG GCCTGCTGGA TTACATCGAC | 1740 |
| AAAGAGATTA TTCCAGATTT CCTGAGTGGG GAGTGCATGT GCGAAGTGCC AGAGGGTGGA | 1800 |
| CTGGTCCCCA AATCTCTGTA CCGGACTGCA GAGGAGCTGG AGAACGAAGA CCTGAAGCTC | 1860 |
| TGGACTGAGA CCATCTACCA GTCTGCAAGC GTCTTCAAAG GAGCCCCACA TGAGATTCTC | 1920 |
| ATTCAGATTG TGGATGCCTC GTCAGTCATC ACTTGGGATT TCGACGTGTG CAAAGGGGAC | 1980 |
| ATTGTGTTTA ACATCTATCA CTCCAAGAGG TCGCCACAAC CACCCAAAAA GGACTCCCTG | 2040 |
| GGAGCCCACA GCATCACCTC TCCGGGTGGG AACAATGTGC AGCTCATAGA CAAAGTCTGG | 2100 |
| CAGCTGGGCC GCGACTACAG CATGGTGGAG TCGCCTCTGA TCTGCAAAGA AGGAGAAAGC | 2160 |
| GTGCAGGGTT CCCATGTGAC CAGGTGGCCG GGCTTCTACA TCCTGCAGTG GAAATTCCAC | 2220 |
| AGCATGCCTG CGTGCGCCGC CAGCAGCCTT CCCCGGGTGG ACGACGTGCT TGCGTCCCTG | 2280 |
| CAGGTCTCTT CGCACAAGTG TAAAGTGATG TACTACACCG AGGTGATCGG CTCGGAGGAT | 2340 |
| TTCAGAGGTT CCATGACGAG CCTGGAGTCC AGCCACAGCG GCTTCTCCCA GCTGAGTGCC | 2400 |
| GCCACCACCT CCTCCAGCCA GTCCCACTCC AGCTCCATGA TCTCCAGGTA GTGCCGCGCT | 2460 |
| GCCTGCACCT AGTGTGCAGA GGGGACGGCC GCCCCTCCTC GGACAGCAGC TGCACCCGCC | 2520 |
| CACCCAGCGG CGACATTGTA CAGACTCCTC TCACCTCTAG ATAGCAAATA GCTCTCAGAT | 2580 |
| GGTAAACGTA GTCGTTTGAT CCCAAAACTA CCTTGGCAGG TAGTTTTAAC TCTGATCCTA | 2640 |
| ACTTAACTCA ATAGCCATAG ATTTTGTATA CGTTGTGCAC AAAATCCAAC CAGAGCGCAA | 2700 |
| GGGCTCTCTT GAAAGAAAAG TAGTTTCTGT ACCAATTAAA GGATTGACGT GGTCTCAGAT | 2760 |
| ATTGATGCAA AAAATTTTTC CAACGAACTC CGCATTGTCC ATTAGTGAAT GAATTCCTGT | 2820 |
| GACATcCTCC AGAGATGGCC CCTCCTCACC TGGGACGGAA GCTGCCAGCT CGCTTCCCCC | 2880 |
| AAGCTGCCTC ATGGCCCGCA CGCCGCCTCA CGGCCCCCAT GCTTCCCGCC AGTCAAGATG | 2940 |
| GTCTGTGGAC TTAGGGCCAG CCCTTGAGGT CCTTATCCTC TGAGGATTCA GAGGTTGCCT | 3000 |
| GCGGAGTACC TTGTCCCAGG GCCAGACACA CCCACACCAC CCACTGTCTG CAGTGGGGCC | 3060 |
| GGGGGCTCAG GAGGGGCTCT CAGGGACTCC TGGTGACTCC AGGAAAATGC TGCCATCGTT | 3120 |
| AAACATTACT TTCTCTTTCC TCCTTTTCAA ATCTTTTTGA TACTTTTTAG AGCAGGATTT | 3180 |
| TTCTGTATGT GAACTTGGGT GGGGGGGTTC TTCCCGTTTC CTTCCGTGCG TCGCCCCTCT | 3240 |
| CACCTGCAGT CAGCTCCCAG CCCAGTGTAG GCCATCTCCT CTGTGCCCTC TGGAGGCTCA | 3300 |
| TTGTCTCAGA GCCCAGACAG TTCCAGCCAC TAGGAGGCCG TCTTGGAACC AGCAAGTCGC | 3360 |
| ATTTGCCACT TGACACTGTC CATGGGGTTT TATTAGTAGC TAAGCAGCAG CTCTCGCATC | 3420 |
| CACTTCAGGG TGGCGTGTGG CATGTAGGAG TCCTGCTTCT TTGTACATGG GAATTGTGGA | 3480 |
| CTCATGCGTG TGTGTGTGTG CATGTGCTGT GTGTGTGCAT GTGTGCATGA CGGTGGGGGT | 3540 |
| GCTGGGGGGA CGGGGTGAGT GGAAACTTAG TTTGAGTAAT GAAGGAATCT TCACAGAAGC | 3600 |
| AAATCAGAAT ATGGGATTTG TTTGCCTTTT ACATTTTGTT TAATTCCTGA TTTTAAAGCC | 3660 |
| TGCTCTATCT GGTACAGGCC CTTATTTTTT CAGCTTTTTA TGGGAAAAGC AGGTTATTTG | 3720 |
| AGAATCTGTC CAGAAGTTGC ATAGGGGATG GCCTCCACGA TAAGGACATG CAACACGTGT | 3780 |
| TTCTGTGTGC AGCAGAGGCC GTGTTTTTCA TGCCAAACCC CACGCGGCTG TCAACTGTGT | 3840 |
| GCGTGGTAGG CATGGAGATC CTGGTTGTGC CGTCTCAGCT CCGCTCTGAA GGCACTGTGT | 3900 |
| GGGTGCTGCG TGACTGGAGA GCTGTGTGGA GGCCATGTGT GCCCCGTGCA GGGATCAGGA | 3960 |
| GGGCGGGGGA GGGACCGAGC AGCCCTCTTG CCCGGTCGGG TCAGCCCTAG TGGCTGCCTG | 4020 |
| CACACTGTAG ACGTCCCAGG GCCTGTGCTG TGATCACCTG CCTTTGGACC ACATTTGTGT | 4080 |
| TTGCTCTTAG AGATCGAGCT CCTCAGTGGT ACCTGAAGCC TTTGCTTCCG GAAAGCGCGG | 4140 |
| TAGGGTTCGT AGGTAGGGCT AGTAGGTAGG GTTAGTAGGT AGGGCTAGTA GGTAGGGCTA | 4200 |
| GTAGGTAGGG TTAGTAGGTA GGGTTCGTAG GTAGGGCTGG TAGGTAGGGT TAGTAGGTAG | 4260 |
| GGCTAGTAGG TAGGGTTCGT AGGTAGGGCT AGTAGGTAGG GTTAGTAGGT AGGGCTAGTA | 4320 |
| GGTAGGGCTA GTAGGTAGGG TTAGTAGGTA GGGTTCGTAG GTAGGGCTGG TAGGTAGGGT | 4380 |
| TAGTAGGTAG GGCTAGTAGG TAGGGTTCGT AGGTAGGGCT AGTAGGTAGG GTTAGTAGGT | 4440 |
| AGGGCTAGTA GGTAGGGCTA GTAGGTAGGG TTAGTAGGTA GGGTTCGTAG GTAGGGCTGG | 4500 |
| TAGGTAGGGT TAGTAGGTAG GGCTAGTAGG TAGGGCTAGT AGGTAGGGCT AGTAGGTAGG | 4560 |
| GTTAGTAGGT AGGGCTAGTA GGTAGGGCTA GTAGGTAGGG TTAGTAGGTA GGGTTCGTAG | 4620 |
| GTAGGGCTGG TAGGTAGGGT TAGTAGGTAG GGCTAGTAGG TAGGGCTAGT AGGTAGGGCT | 4680 |
| AGTAGGTAGG GCTAGTAGGT AGGGCTAGTA GGTAGGGCTA GTAGGTAGGG CTAGTAGGTA | 4740 |
| GGGTTCGTAG GTAGGGTTCG TAGGTAGGGT TCGTAGGTAG GGTTAGTAGC GCGTCTGTGC | 4800 |
| TGCTTCCACC TGGTGCTTCC TGTTCCCAAA TCACAAGGGC CTGAAGGTGG TCCCTGCTTT | 4860 |
| CTCTTTCTCT TTCTCTGTGT CTCAGATGGC GATTTTGCTG ACAGCTGCCA AGAAAATGCT | 4920 |
| TCACTCAACA GTCCTCATGT GCCCAGAGAT GTTTATAGAA CTGTTTGAAT TGCAGCCATC | 4980 |
| CCCTGCCCCC TCCCAGGCTG AAGATCTGTT CTTTTTAAGT TGATTCGGGA GTGGCATTCT | 5040 |
| TTTATACCCA AAGACTGTAG TGCATCTTGA AGAGCTCAAA GCACATGACC GCACAAATGC | 5100 |
| TTACAGGGTT TCCTCCCGAG TAATCCAATC TCACTCCCCT TGTAAGGGAA TTCTGGGGCA | 5160 |
| GCTATGGTTT GAGTATGCAG TTTGCATCGT GTTTCTACCT TTAGTACCTT GCCACTCTTT | 5220 |
| TAAAACGCTG CTGTCATTTC CCATTTCTTA GTACTAATGA TTCTTTGATT CTCCCTCTAT | 5280 |
| TATGTCTTAA TTCACTTTCC TTCCTAAATT TGTTATTTGC ATATCAAATT CTGTAAATGT | 5340 |
| TTTGTAAACA TATTACCTCA CTTGGTAATA CAATACTGAT AGTCTTTAAA AGATTTTTTT | 5400 |
| ATTGTTATCA ATAATAAATG TGAACTATTT AAAG | |

Seq ID No: 137 Protein sequence:
Protein Accession #: NP_002994.1

| | |
|---|---|
| 1 11 21 31 41 51 | |
| \| \| \| \| \| \| | |
| MVQKYQSPVR VYKYPFELIM AAYERRFPTC PLIPMFVGSD TVSEFKSEDG AINVIERRCK | 60 |
| LDVDAPRLLK KIAGVDYVYF VQKNSLNSRE RTLHIEAYNE TFSNRVIINE HCCYTVMPEN | 120 |
| EDWTCFEQSA SLDIKSFFGF ESTVEKIANK QYTSNIKKGK EIIEYYLRQL EEEGITFVPR | 180 |
| WSPPSITPSS ETSSSSSKKQ AASMAVVIPE AALKEGLSGD ALSSPSAPEP VVGTPDDKLD | 240 |
| ADHIKRYLGD LTPLQESCLI RLRQWLQETH KGKIPKDEHI LRFLRARDFN IDKAREIMCQ | 300 |
| SLTWRKQHQV DYILETWTPP QVLQDYYAGG WHHHDKDGRP LYVLRLGQMD TKGLVRALGE | 360 |
| EALLRYVLSV NEERLRRCEE NTKVFGRPIS SWTCLVDLEG LNMRRLWRPG VKALLRIIEV | 420 |
| VEANYPETLG RLLILRAPRV FPVLWTLVSP FIDDNTRRKF LITAGNGYQG PGGLLDYIDK | 480 |
| EIIPDFLSGE CMCEVPEGGL VPKSLYRTAE ELENEDLKLW TETIYQSASV FKGAPHEILI | 540 |
| QIVDASSVIT WDFDVCKGDI VFNIYHSKRS PQPPKKDSLG AHSITSPGGN NVQLIDKVWQ | 600 |
| LGRDYSMVES PLICKEGESY QGSHVTRWPG FYILQWKFHS MPACAASSLP RVDDVLASLQ | 660 |
| VSSHKCKVMY YTEVIGSEDF RGSMTSLESS HSGFSQLSAA TTSSSQSHSS SMISR | |

Seq ID NO: 138 DNA sequence:
Nucleic Acid Accession #: NM_004181.1
Coding sequence: 32-670 (underlined sequences correspond to start and
stop codons)

| | |
|---|---|
| 1 11 21 31 41 51 | |
| \| \| \| \| \| \| | |
| GCAGAAATAG CCTAGGGAGA TCAACCCCGA GATGCTGAAC AAAGTGCTGT CCCGGCTGGG | 60 |
| GGTCGCCGGC CAGTGGCGCT TCGTGGACGT GCTGGGGCTG GAAGAGGAGT CTCTGGGCTC | 120 |
| GGTGCCAGCG CCTGCCTGCG CGCTGCTGCT GCTGTTTCCC CTCACGGCCC AGCATGAGAA | 180 |
| CTTCAGGAAA AAGCAGATTG AAGAGCTGAA GGGACAAGAA GTTAGTCCTA AAGTGTACTT | 240 |
| CATGAAGCAG ACCATTGGGA ATTCCTGTGG CACAATCGGA CTTATTCACG CAGTGGCCAA | 300 |
| TAATCAAGAC AAACTGGGAT TTGAGGATGG ATCAGTTCTG AAACAGTTTC TTTCTGAAAC | 360 |
| AGAGAAAATG TCCCCTGAAG ACAGAGCAAA ATGCTTTGAA AAGAATGAGG CCATACAGGC | 420 |
| AGCCCATGAT GCCGTGGCAC AGGAAGGCCA ATGTCGGGTA GATGACAAGG TGAATTTCCA | 480 |
| TTTTATTCTG TTTAACAACG TGGATGGCCA CCTCTATGAA CTTGATGGAC GAATGCCTTT | 540 |
| TCCGGTGAAC CATGGCGCCA GTTCAGAGGA CACCCTGCTG AAGGACGCTG CCAAGGTGTG | 600 |
| CAGAGAATTC ACCGAGCGTG AGCAAGGAGA AGTCCGCTTC TCTGCCGTGG CTCTCTGCAA | 660 |
| GGCAGCCTAA TGCTCTGTGG GAGGGACTTT GCTGATTTCC CCTCTTCCCT TCAACATGAA | 720 |
| AATATATACC CCCCATGCAG TCTAAAATGC TTCAGTACTT GTGAAACACA GCTGTTCTTC | 780 |
| TGTTCTGCAG ACACGCCTTC CCCTCAGCCA CACCCAGGCA CTTAAGCACA AGCAGAGTGC | 840 |
| ACAGCTGTCC ACTGGGCCAT TGTGGTGTGA GCTTCAGATG GTGAAGCATT CTCCCCAGTG | 900 |
| TATGTCTTGT ATCCGATATC TAACGCTTTA AATGGCTACT TTGGTTTCTG TCTGTAAGTT | 960 |
| AAGACCTTGG ATGTGGTTAT GTTGTCCTAA AGAATAAATT TTGCTGATAG TAGC | |

Seq ID No: 139 Protein sequence:
Protein Accession #: NP_004172.1

| | |
|---|---|
| 1 11 21 31 41 51 | |
| \| \| \| \| \| \| | |
| MLNKVLSRLG VAGQWRFVDV LGLEEESLGS VPAPACALLL LFPLTAQHEN FRKKQIEELK | 60 |
| CQEVSPKVYF MKQTIGNSCG TIGLIHAVAN NQDKLGFEDG SVLKQFLSET EKMSPEDRAK | 120 |
| CFEKNEAIQA AHDAVAQEGQ CRVDDRVNFH FILFNNVDGH LYELDGRMPE PVNHGASSED | 180 |
| TLLKDAAKVC REFTEREQGE VRFSAVALCK AA | |

Seq ID NO: 140 DNA sequence:
Nucleic Acid Accession #: NM_000201.1
Coding sequence: 58-1656 (underlined sequences correspond to start and stop codons)

| | |
|---|---|
| 1 11 21 31 41 51 | |
| \| \| \| \| \| \| | |
| GCGCCCCAGT CGACGCTGAG CTCCTCTGCT ACTCAGAGTT GCAACCTCAG CCTCGCTATG | 60 |
| GCTCCCAGCA GCCCCCGGCC CGCGCTGCCC GCACTCCTGG TCCTGCTCGG GGCTCTGTTC | 120 |
| CCAGGACCTG GCAATGCCCA GACATCTGTG TCCCCCTCAA AAGTCATCCT GCCCCGGGGA | 180 |
| GGCTCCGTGC TGGTGACATG CAGCACCTCC TGTGACCAGC CCAAGTTGTT GGGCATAGAG | 240 |
| ACCCCGTTGC CTAAAAAGGA GTTGCTCCTG CCTGGGAACA ACCGGAAGGT GTATGAACTG | 300 |
| AGCAATGTGC AAGAAGATAG CCAACCAATG TGCTATTCAA ACTGCCCTGA TGGGCAGTCA | 360 |
| ACAGCTAAAA CCTTCCTCAC CGTGTACTGG ACTCCAGAAC GGGTGGAACT GGCACCCCTC | 420 |
| CCCTCTTGGC AGCCAGTGGG CAAGAACCTT ACCCTACGCT GCCAGGTGGA GGGTGGGGCA | 480 |
| CCCCGGGCCA ACCTCACCGT GGTGCTGCTC CGTGGGGAGA AGGAGCTGAA ACGGGAGCCA | 540 |
| GCTGTGGGGG AGCCCGCTGA GGTCACGACC ACGGTGCTGG TGAGGAGAGA TCACCATGGA | 600 |
| GCCAATTTCT CGTGCCGCAC TGAACTGGAC CTGCGGCCCC AAGGGCTGGA GCTGTTTGAG | 660 |
| AACACCTCGG CCCCCTACCA GCTCCAGACC TTTGTCCTGC CAGCGACTCC CCCACAACTT | 720 |
| GTCAGCCCCC GGGTCCTAGA GGTGGACACG CAGGGGACCG TGGTCTGTTC CCTGGACGGG | 780 |
| CTGTTCCCAG TCTCGGAGGC CCAGGTCCAC CTGGCACTGG GGGACCAGAG GTTGAACCCC | 840 |
| ACAGTCACCT ATGGCAACGA CTCCTTCTCG GCCAAGGCCT CAGTCAGTGT GACCGCAGAG | 900 |
| GACGAGGGCA CCCAGCGGCT GACGTGTGCA GTAATACTGG GGAACCAGAG CCAGGAGACA | 960 |
| CTGCAGACAG TGACCATCTA CAGCTTTCCG GCGCCCAACG TGATTCTGAC GAAGCCAGAG | 1020 |
| GTCTCAGAAG GGACCGAGGT GACAGTGAAG TGTGAGGCCC ACCCTAGAGC CAAGGTGACG | 1080 |
| CTGAATGGGG TTCCAGCCCA GCCACTGGGC CCGAGGGCCC AGCTCCTGCT GAAGGCCACC | 1140 |
| CCAGAGGACA ACGGGCGCAG CTTCTCCTGC TCTGCAACCC TGGAGGTGGC CGGCCAGCTT | 1200 |
| ATACACAAGA ACCAGACCCG GGAGCTTCGT GTCCTGTATG GCCCCCGACT GGACGAGAGG | 1260 |
| GATTGTCCGG GAAACTGGAC GTGGCCAGAA AATTCCCAGC AGACTCCAAT GTGCCAGGCT | 1320 |
| TGGGGGAACC CATTGCCCGA GCTCAAGTGT CTAAAGGATG GCACTTTCCC ACTGCCCATC | 1380 |
| GGGGAATCAG TGACTGTCAC TCGAGATCTT GAGGGCACCT ACCTCTGTCG GGCCAGGAGC | 1440 |
| ACTCAAGGGG AGGTCACCCG CGAGGTGACC GTGAATGTGC TCTCCCCCCG GTATGAGATT | 1500 |
| GTCATCATCA CTGTGGTAGC AGCCGCAGTC ATAATGGGCA CTGCAGGCCT CAGCACGTAC | 1560 |
| CTCTATAACC GCCAGCGGAA GATCAAGAAA TACAGACTAC AACAGGCCCA AAAAGGGACC | 1620 |
| CCCATGAAAC CGAACACACA AGCCACGCCT CCCTGAACCT ATCCCGGGAC AGGGCCTCTT | 1680 |
| CCTCGGCCTT CCCATATTGG TGGCAGTGGT GCCACACTGA ACAGAGTGGA AGACATATGC | 1740 |
| CATGCAGCTA CACCTACCGG CCCTGGGACG CCGGAGGACA GGGCATTGTC CTCAGTCAGA | 1800 |
| TACAACAGCA TTTGGGGCCA TGGTACCTGC ACACCTAAAA CACTAGGCCA CGCATCTGAT | 1860 |
| CTGTAGTCAC ATGACTAAGC CAAGAGGAAG GAGCAAGACT CAAGACATGA TTGATGGATG | 1920 |
| TTAAAGTCTA GCCTGATGAG AGGGGAAGTG GTGGGGGAGA CATAGCCCCA CCATGAGGAC | 1980 |
| ATACAACTGG GAAATACTGA AACTTGCTGC CTATTGGGTA TGCTGAGGCC CACAGACTTA | 2040 |
| CAGAAGAAGT GGCCCTCCAT AGACATGTGT AGCATCAAAA CACAAAGGCC CACACTTCCT | 2100 |
| GACGGATGCC AGCTTGGGCA CTGCTGTCTA CTGACCCCAA CCCTTGATGA TATGTATTTA | 2160 |
| TTCATTTGTT ATTTTACCAG CTATTTATTG AGTGTCTTTT ATGTAGGCTA AATGAACATA | 2220 |
| GGTCTCTGGC CTCACGGAGC TCCCAGTCCA TGTCACATTC AAGGTCACCA GGTACAGTTG | 2280 |
| TACAGGTTGT ACACTGCAGG AGAGTGCCTG GCAAAAAGAT CAAATGGGGC TGGGACTTCT | 2340 |
| CATTGGCCAA CCTGCCTTTC CCCAGAAGGA GTGATTTTTC TATCGGCACA AAAGCACTAT | 2400 |
| ATGGACTGGT AATGGTTCAC AGGTTCAGAG ATTACCCAGT GAGGCCTTAT TCCTCCCTTC | 2460 |
| CCCCCAAAAC TGACACCTTT GTTAGCCACC TCCCCACCCA CATACATTTC TGCCAGTGTT | 2520 |
| CACAATGACA CTCAGCGGTC ATGTCTGGAC ATGAGTGCCC AGGGAATATG CCCAAGCTAT | 2580 |
| GCCTTGTCCT CTTGTCCTGT TTGCATTTCA CTGGGAGCTT GCACTATTGC AGCTCCAGTT | 2640 |
| TCCTGCAGTG ATCAGGGTCC TGCAAGCAGT GGGGAAGGGG GCCAAGGTAT TGGAGGACTC | 2700 |
| CCTCCCAGCT TTGGAAGGGT CATCCGCGTG TGTGTGTGTG TGTATGTGTA GACAAGCTCT | 2760 |
| CGCTCTGTCA CCCAGGCTGG AGTGCAGTGG TGCAATCATG GTTCACTGCA GTCTTGACCT | 2820 |
| TTTGGGCTCA AGTGATCCTC CCACCTCAGC CTCCTGAGTA GCTGGGACCA TAGGCTCACA | 2880 |
| ACACCACACC TGGCAAATTT GATTTTTTTT TTTTTTTTCA GAGACGGGGT CTCGCAACAT | 2940 |
| TGCCCAGACT TCCTTTGTGT TAGTTAATAA AGCTTTCTCA ACTGCC | |

Seq ID NO: 141 Protein sequence:
Protein Accession #: NP_000192.1

| | |
|---|---|
| 1 11 21 31 41 51 | |
| \| \| \| \| \| \| | |
| MLQFVRAGAR AWLRPTGSQG LSSLAEEAAR ATENPEQVAS EGLPEPVLRK VELPVPTHRR | 60 |
| PVQAWVESLR GFEQERVGLA DLHPDVFATA PRLDILHQVA MWQKNFKRIS YAKTKTRAEV | 120 |
| RGGGGKPLAA ERHWAGPAWQ HPLSALARRR CCPWPPGPTS YYYMLPMKVR ALGLKVALTV | 180 |
| KLAQDDLHIM DSLELPTGDP QYLTELAHYR RWGDSVLLVD LTHEEMPQSI VEATSRLKTF | 240 |
| NLIPAVGLNV HSMLKHQTLV LTLPTVAFLE DKLLWQDSRY RPLYPFSLPY SDFPRPLPHA | 300 |
| TQGPAATPYH C | |

Seq ID NO: 142 DNA sequence:
Nucleic Acid Accession #: NM_000270.1
Coding sequence: 110-979 (underlined sequences correspond to start and stop codons)

| | |
|---|---|
| 1 11 21 31 41 51 | |
| \| \| \| \| \| \| | |
| AACTGTGCGA ACCAGACCCG GCAGCCTTGC TCAGTTCAGC ATAGCGGAGC GGATCCGATC | 60 |
| GGATCGGAGC ACACCGGAGC AGGCTCATCG AGAAGGCGTC TGCGAGACCA TGGAGAACGG | 120 |
| ATACACCTAT GAAGATTATA AGAACACTGC AGAATGGCTT CTGTCTCATA CTAAGCACCG | 180 |
| ACCTCAAGTT GCAATAATCT GTGGTTCTGG ATTAGGAGGT CTGACTGATA AATTAACTCA | 240 |
| GGCCCAGATC TTTGACTACA GTGAAATCCC CAACTTTCCT CGAAGTACAG TGCCAGGTCA | 300 |
| TGCTGGCCGA CTGGTGTTTG GGTTCCTGAA TGGCAGGGCC TGTGTGATGA TGCAGGGCAG | 360 |
| GTTCCACATG TATGAAGGGT ACCCACTCTG GAAGGTGACA TTCCCAGTGA GGGTTTTCCA | 420 |
| CCTTCTGGGT GTGGACACCC TGGTAGTCAC CAATGCAGCA GGAGGGCTGA ACCCCAAGTT | 480 |
| TGAGGTTGGA GATATCATGC TGATCCGTGA CCATATCAAC CTACCTGGTT TCAGTGGTCA | 540 |
| GAACCCTCTC AGAGGGCCCA ATGATGAAAG GTTTGGAGAT CGTTTCCCTG CCATGTCTGA | 600 |
| TGCCTACGAC CGGACTATGA GGCAGAGGGC TCTCAGTACC TGGAAACAAA TGGGGGAGCA | 660 |
| ACGTGAGCTA CAGGAAGGCA CCTATGTGAT GGTGGCAGGC CCCAGCTTTG AGACTGTGGC | 720 |
| AGAATGTCGT GTGCTGCAGA AGCTGGGAGC AGACGCTGTT GGCATGAGTA CAGTACCAGA | 780 |
| AGTTATCGTT GCACGGCACT GTGGACTTCG AGTCTTTGGC TTCTCACTCA TCACTAACAA | 840 |
| GGTCATCATG GATTATGAAA GCCTGGAGAA GGCCAACCAT GAAGAAGTCT TAGCAGCTGG | 900 |
| CAAACAAGCT GCACAGAAAT TGGAACAGTT TGTCTCCATT CTTATGGCCA GCATTCCACT | 960 |
| CCCTGACAAA GCCAGTTGAC CTGCCTTGGA GTCGTCTGGC ATCTCCCACA CAAGACCCAA | 1020 |
| GTAGCTGCTA CCTTCTTTGG CCCCTTGCTG GAGTCATGTG CCTCTGTCCT TAGGTTGTAG | 1080 |
| CAGAAAGGAA AAGATTCCTG TCCTTCACCT TTCCCACTTT CTTCTACCAG ACCCTTCTGG | 1140 |
| TGCCAGATCC TCTTCTCAAA GCTGGGATTA CAGGTGTGAG CATAGTGAGA CCTTGGCGCT | 1200 |
| ACAAAATAAA GCTGTTCTCA TTCCTGTTCT TTCTTACACA AGAGCTGGAG CCCGTGCCCT | 1260 |
| ACCACACATC TGTGGAGATG CCCAGGATTT GACTCGGGCC TTAGAACTTT GCATAGCAGC | 1320 |
| TGCTACTAGC TCTTTGAGAT AATACATTCC GAGGGGCTCA GTTCTGCCTT ATCTAAATCA | 1380 |
| CCAGAGACCA AACAAGGACT AATCCAATAC CTCTTGGA | |

Seq ID No: 143 Protein sequence:
Protein Accession #: NP_000261.1

| | |
|---|---|
| 1 11 21 31 41 51 | |
| \| \| \| \| \| \| | |
| MENGYTYEDY KNTAEWLLSH TKHRPQVAII CGSGLGGLTD KLTQAQIFDY SEIPNFPRST | 60 |
| VPGHAGRLVF GFLNGRACVM MQORFHMYEG YPLWKVTFPV RVFHLLGVDT LVVTNAAGGL | 120 |
| NPKFEVGDIM LIRDHINLPG FSGQNPLRGP NDERFGDRFP AMSDAYDRTM RQRALSTWKQ | 180 |
| MGEQRELQEG TYVMVAGPSF ETVAECRVLQ KLGADAVGMS TYPEVIVARM CGLRVFGFSL | 240 |
| ITNKVIMDYE SLEKANHEEV LAAGKQAAQK LEQFVSILMA SIPLPDKAS | |

Seq ID ND: 144 DNA sequence:
Nucleic Acid Accession #: NM_015577.1
Coding sequence: 112-3054 (underlined sequences correspond to start and stop codons)

| | |
|---|---|
| 1 11 21 31 41 51 | |
| \| \| \| \| \| \| | |
| GAAGCGGCGG GCGGGGTGGA GCAGCCAGCT GGGTCCGGGG AGCGCCGCCG CCGCCTCGAT | 60 |
| GGGGTGTTGA AAAGTCTCCT CTAGAGCTTT GGAAGGCTGA ATGCACTAAA CATGAAGAGC | 120 |
| TTGAAAGCGA AGTTCAGGAA GAGTGACACC AATGAGTGGA ACAAGAATGA TGACCGGCTA | 180 |
| CTGCAGGCCG TGGAGAATGG AGATGCGGAG AAGGTGGCCT CACTGCTCGG CAAGAAGGGG | 240 |
| GCCAGTGCCA CCAAACACGA CAGTGAGGGC AAGACCGCTT TCCATCTTGC TGCTGCAAAA | 300 |
| GGACACGTGG AATGCCTCAG GGTCATGATT ACACATGGTG TGGATGTGAC AGCCCAAGAT | 360 |
| ACTACCGGAC ACAGCGCCTT ACATCTCGCA GCCAAGAACA GCCACCATGA ATGCATCAGG | 420 |
| AGGCTGCTTC AGTCTAAATG CCCAGCCGAA AGTGTCGACA GCTCTGGGAA AACAGCTTTA | 480 |
| CATTATGCAG CGGCTCAGGG CTGCCTTCAA GCTGTGCAGA TTCTCTGCGA ACACAAGAGC | 540 |
| CCCATAAACC TCAAAGATTT GGATGGGAAT ATACCGCTGC TTCTTGCTGT ACAAAATGGT | 600 |
| CACAGTGAGA TCTGTCACTT TCTCCTGGAT CATGGAGCAG ATGTCAATTC CAGGAACAAA | 660 |
| AGTGGAAGAA CTGCTCTCAT GCTGGCCTGT GAGATTGGCA GCTCTAACGC TGTGGAAGCC | 720 |
| TTAATTAAAA AGGGTGCAGA CCTAAACCTT GTAGATTCTC TTGGATACAA TGCCTTACAT | 780 |
| TATTCCAAAC TCTCAGAAAA TGCAGGAATT CAAAGCCTTC TATTATCAAA AATCTCTCAG | 840 |
| GATGCTGATT TAAAGACCCC AACAAAACCA AAGCAGCATG ACCAAGTCTC TAAAATAAGC | 900 |
| TCAGAAAGAA GTGGAACTCC AAAAACACGC AAAGCTCCAC CACCTCCTAT CAGTCCTACC | 960 |
| CAGTTGAGTG ATGTCTCTTC CCCAAGATCA ATAACTTCGA CTCCACTATC GGGAAAGGAA | 1020 |
| TCGGTATTTT TTGCTGAACC ACCCTTCAAG GCTGAGATCA GTTCTATACG AGAAAACAAA | 1080 |
| GACAGACTAA GTGACAGTAC TACAGGTGCT GATAGCTTAT TGGATATAAG TTCTGAAGCT | 1140 |
| GACCAACAAG ATCTTCTCTC TCTATTGCAA GCAAAAGTTG CTTCCCTTAC CTTACACAAT | 1200 |
| AAGGAGTTAC AAGATAAATT ACAGGCCAAA TCACCCAAGG AGGCGGAAGC AGACCTAACC | 1260 |
| TTTGACTCAT ACCATTCCAC CCAAACTGAC TTGGGCCCAT CCCTGGGAAA ACCTGGTGAA | 1320 |
| ACCTCTCCCC CAGACTCCAA ATCATCTCCA TCTGTCTTAA TACATTCTTT AGGTAAATCC | 1380 |
| ACTACTGACA ATGATGTCAG AATTCAGCAA CTGCAAGAGA TTTTGCAAGA TCTACAGAAG | 1440 |
| AGATTAGAGA GCTCTGAAGC AGAGAGAAAA CAGCTACAGG TCGAACTCCA ATCCCGAAGG | 1500 |
| GCAGAACTGG TATGCTTAAA CAACACTGAG ATTTCAGAGA ACAGCTCTGA CCTCAGCCAG | 1560 |
| AAACTTAAAG AAACTCAGAG CAAATACGAG GAGGCTATGA AAGAAGTCCT TAGTGTGCAG | 1620 |
| AAGCAGATGA AACTCGGTCT TGTCTCACCT GAAAGCATGG ATAATTATTC ACATTTCCAC | 1680 |
| GAGCTGAGGG TCACGGAAGA GGAAATAAAT GTGCTAAAGC AGGATCTGCA GAATGCATTA | 1740 |
| GAAGAAAGTG AAAGAAATAA AGAGAAAGTG AGAGAGTTAG AGGAAAAACT GGTAGAGAGG | 1800 |
| GAGAAAGGTA CAGTGATTAA GCCACCTGTG GAAGAGTACG AGGAAATGAA AAGTTCATAT | 1860 |
| TGCTCTGTTA TTGAGAATAT GAATAAGGAG AAAGCATTTT TGTTTGAGAA ATACCAAGAA | 1920 |
| GCCCAAGAAG AAATCATGAA ATTAAAAGAC ACACTAAAAA GTCAGATGAC ACAGGAAGCC | 1980 |
| AGTGATGAAG CTGAGGACAT GAAAGAAGCC ATGAATAGGA TGATAGATGA ACTCAATAAA | 2040 |
| CAGGTGAGCG AGCTGTCACA GCTGTACAAA GAAGCCCAGG CTGAGCTGGA GGATTACAGG | 2100 |
| AAGAGGAAAT CTCTAGAGGA TGTCACAGCT GAATATATCC ATAAAGCAGA GCATGAGAAA | 2160 |
| CTGATGCAAT TGACAAACGT GTCCAGGGCT AAAGCAGAAG ATGCACTGTC TGAAATGAAG | 2220 |
| TCTCAGTATT CAAAAGTGTT GAATGAGTTG ACCCAGCTCA AACAACTGGT GGATGCACAA | 2280 |
| AAAGAGAACT CTGTCTCTAT CACAGAACAT TTGCAAGTGA TAACCACGCT GCGGACTGCA | 2340 |
| GCAAAAGAGA TGGAAGAAAA AATAAGCAAT CTTAAGGAAC ACCTTGCAAG CAAGGAAGTG | 2400 |
| GAAGTAGCAA AGCTGGAGAA ACAACTCTTA GAAGAGAAAG CTGCTATGAC TGATGCAATG | 2460 |
| GTACCTCGGT CTTCCTATGA AAAACTCCAG TCATCCTTAG AGAGTGAAGT GAGTGTGTTG | 2520 |
| GCATCGAAAT TAAAGGAATC TGTGAAAGAG AAAGAGAAGG TCCATTCAGA GGTTGTCCAG | 2580 |
| ATTAGAAGTG AGGTCTCACA GGTGAAAAGA GAAAAGGAAA ATATTCAGAC TCTCTTGAAA | 2640 |
| TCCAAAGAGC AAGAAGTAAA TGAACTTCTG CAAAAATTCC AGCAAGCTCA GGAAGAACTT | 2700 |
| GCAGAAATGA AAAGATACGC TGAGAGCTCT TCAAAACTGG AGGAAGATAA AGATAAAAAG | 2760 |
| ATAAATGAGA TGTCGAAGGA AGTCACCAAA TTGAAGGAGG CCTTGAACAG CCTCTCCCAG | 2820 |
| CTCTCCTACT CAACAAGCTC ATCCAAAAGG CAGAGTCAGC AGCTGGAGGC GCTGCAGCAG | 2880 |
| CAAGTCAAAC AGCTCCAGAA CCAGCTGGCG GAATGCAAGA AACAACACCA GGAGGTCATA | 2940 |
| TCAGTTTACA GAATGCATCT TCTGTATGCT GTGCAGGGCC AGATGGATGA AGATGTCCAG | 3000 |
| AAAGTACTGA AGCAAATCCT TACCATGTGT AAAAACCAGT CTCAAAAGAA GTAAAGTGGA | 3060 |
| TTCCTTGGCA GGACACTGCC CCTTGTCATC TGTCTTTGTG TTAGATCCAG AGTTGTCGGC | 3120 |
| AGCCGCTGCC ATTGTTCTCA TTCGTGGTAT GCACTGTGGC CTAGCGTAGC TTCTTCCCTT | 3180 |
| TCCAAAGGTT TCTGAGGACT TCTCCCAGGA GAAGACTGCC CGCCTCAGAA CTGCTTAGAG | 3240 |
| ACTTCAAACC AGCAGAGGTG AAAGTCCCTG TCATCCCTTC AGATTCCAGA GCTGGGATCA | 3300 |
| GCCATGCCCA GAGGTCTGGT CCTGATGCTG GCAGGGGGGC CCCCTCCTCC ATCCCTGACT | 3360 |
| GGCTGAGTGG CTTTATCACC ACCGAGTGAT GTGCTGAGGC CTCCTGCAGT GAATGCTCCT | 3420 |
| TCCATTCCTG TACTCGGGCA GTGCCATTCA GCACAGGAGA GCTCTTTTTG CCTTTGGCTT | 3480 |
| TCAATTCCAA AACATGATTT AATTTCTAAC TAAATTAGTA TGGCACTAGT TATGAAGTAT | 3540 |
| CTGCTTAAAA CCCTTCATCA TGATATCCTG TGGATTTAAA AACTCTAATT CCATGTTTTC | 3600 |
| TTCCCATCTG CCTTATATAT CTCATCACCC TGCTTATCAA TATTCAGTTT GATGAGCACT | 3660 |
| ATTAACTAAA ATATGAAACT TAAAAACAAA AGCAAGTTGT CCTTAAAAGT TCTTTTTTTA | 3720 |
| AGTAAATTGT TGACATACTG CAAATTTTCT ATGCAAACTT GCCTCCTGCT GTTATCTGTG | 3780 |
| AAGCTCAGGA AATCCAAACA TTTGTGTTTC AACAAGGGAC AGTAAACTGT GTGTTTACAG | 3840 |
| CCAAAAGAAA TGCCTCATAG TTCTTAACCT CAACTTTTGT AGAAGTATTT TTTTCTCTGT | 3900 |
| AATATTTTTA TTGGCTCATA AAGATGTTTT CATATCTGAA CTCCTAAATA AGTGAAATTA | 3960 |
| CAGTAGATTA TATTAACAAA ATACTTTTTA GGTAGCCATG CTTGAGACTT TTTAAAAATA | 4020 |
| TAACTTTTTC CTTAAAGTTT TCAGCTATAG CAAAAGGTAG TTATGTATGC CAGACCTAAT | 4080 |
| ATGAGCTGCC ACCAACACCC CTAGAACTTT CAGCCATGGT GTCTTCAGAA TTGTAGCGCA | 4140 |
| TTTCTGAATC TAGCAAATCC TCCTTTTACC CGTTGAATGT TTTGAATGCC CTGACTCTAC | 4200 |
| CAGCGCCCAT AAATGATCTC TAGAAGGACT GTTAGTACCA ATCTGTTTTT CAACTTTGAA | 4260 |
| GCTAAAAACC CTGATATGGT AATATTATGG TGCATAGCAG AGGTCTCGGA AAAAAAATAT | 4320 |
| TTCTGTTCAC TTTACTTTCA GGTTAAAAAT GTTTCTAACA CGCTTGCAAC TTCCCTTATG | 4380 |
| GCATTAATCT TGTTGAGGGA GAGAGACAGA ATCCTGGACT CTCCAAAGTA TTTAACTGAA | 4440 |
| AGTAGGGCCT GCTCTGACAG GGCCCATGTC CCACAAGGCT GCTTGGCCTC AGTGGGTGCT | 4500 |
| TGGCTGTGCT GGATGATATG TTGATCTGTA TTGGATAAGG ACCAATGACA GCAAAGCAAA | 4560 |
| AATGGCTTTA AAGCTTGGTG TTACTTTTCT TAAGTTGTTT AATTATAGTT AAGCAATTTC | 4620 |
| AAAAATGCTC CAAAGAAATG TGAAAGGACC TTTTGTCACA GCACTTCAGA AAATACACAA | 4680 |
| CAGCCCCTTC TGCCCCCGCA CAGAAATGCT GCAGAGTATA TAAAACTTGA GACATTTTTG | 4740 |
| TAGGATGCCT GACGAGGTGT AGCCTTTTAT CTTGTTTCCG GATGCATATT TATTACGAGT | 4800 |
| ACTCTGGTTA AATATTGAAA AGTTATATGC TGTAGTTTTT AGTATTTTGT CTTTGTAATT | 4860 |
| TACAGAAGTT ATTGGAGAAA ATAAACTTGT TTCATTTTGC AAAAAAAAAA AAAAAAAAAA | 4920 |

Seq ID No: 145 Protein sequence;
Protein Accession #: NP_056392.1

| | |
|---|---|
| 1 11 21 31 41 51 | |
| \| \| \| \| \| \| | |
| MKSLKAKFRK SDTNEWNKND DRLLQAVENG DAEKVASLLG KKGASATKHD SEGKTAFHLA | 60 |
| AAKGHVECLR VMITHGVDVT AQDTTGHSAL HLAAKNSHHE CIRRLLQSKC PAESVDSSGK | 120 |
| TALHYAAAQG CLQAVQILCE HKSPINLKDL DGNIPLLLAV QNGHSEICHF LLDHGADVNS | 180 |
| RNKSGRTALM LACEIGSSNA VEALIKKGAD LNLVDSLGYN ALHYSKLSEN AGIQSLLLSK | 240 |
| ISQDADLKTP TKPKQHDQVS KISSERSGTP KTRKAPPPPI SPTQLSDVSS PRSITSTPLS | 300 |
| GKESVFFAEP PFKAEISSIR ENKDRLSDST TCADSLLDIS SEADQQDLLS LLQAKVASLT | 360 |
| LHNKELQDKL QAKSPKEAEA DLSFDSYHST QTDLGPSLGK PGETSPPDSK SSPSVLIHSL | 420 |
| GKSTTDNDVR IQQLQEILQD LQKRLESSEA ERKQLQVELQ SRRAELVCLN NTEISENSSD | 480 |
| LSQKLKETQS KYEEAMKEVL SVQKQMKLGL VSPESMDNYS HFHELRVTEE EINVLKQDLQ | 540 |
| NALEESERNK EKVRELEEKL VEREKGTVIK PPVEEYEEMK SSYCSVIENM NKEKAFLFEK | 600 |
| YQEAQEEIMK LKDTLKSQMT QEASDEAEDM KEAMNRMIDE LNKQVSELSQ LYKEAQAELE | 660 |
| DYRKRKSLED VTAEYIHKAE NEKLMQLTNV SRAKAEDALS EMKSQYSKVL NELTQLKQLV | 720 |
| DAQKENSVSI TEHLQVITTL RTAAKEMEEK ISNLKEHLAS KEVEVAKLEK QLLEEKAANT | 780 |
| DAMVPRSSYE KLQSSLESEV SVLASKLKES VKEKEKVHSE VVQIRSEVSQ VKREKENIQT | 840 |
| LLKSKEQEVN ELLQKFQQAQ EELAEMKRYA ESSSKLEEDK DKKINEMSKE VTKLKEALNS | 900 |
| LSQLSYSTSS SKRQSQQLEA LQQQVKQLQN QLAECKKQHQ EVISVYRMHL LYAVQGQMDE | 960 |
| DVQKVLKQIL TMCKNQSQKK | |

Seq ID NO: 146 DNA sequence:
Nucleic Acid Accession #: NM_000459.1
Coding sequence: 149-3523 (underlined sequences correspond to start and stop codons)

| | |
|---|---|
| 1 11 21 31 41 51 | |
| \| \| \| \| \| \| | |
| CTTCTGTGCT GTTCCTTCTT GCCTCTAACT TGTAAACAAG ACGTACTAGG ACGATGCTAA | 60 |
| TGGAAAGTCA CAAACCGCTG GGTTTTTGAA AGGATCCTTG GGACCTCATG CACATTTGTG | 120 |
| GAAACTGGAT GGAGAGATTT GGGGAAGCAT GGACTCTTTA GCCAGCTTAG TTCTCTGTGG | 180 |
| AGTCAGCTTG CTCCTTTCTG GAACTGTGGA AGGTGCCATG GACTTGATCT TGATCAATTC | 240 |
| CCTACCTCTT GTATCTGATG CTGAAACATC TCTCACCTGC ATTGCCTCTG GGTGGCGCCC | 300 |
| CCATGAGCCC ATCACCATAG GAAGGGACTT TGAAGCCTTA ATGAACCAGC ACCAGGATCC | 360 |
| GCTGGAAGTT ACTCAAGATG TGACCAGAGA ATGGGCTAAA AAAGTTGTTT GGAAGAGAGA | 420 |
| AAAGGCTAGT AAGATCAATG GTGCTTATTT CTGTGAAGGG CGAGTTCGAG GAGAGGCAAT | 480 |
| CAGGATACGA ACCATGAAGA TGCGTCAACA AGCTTCCTTC CTACCAGCTA CTTTAACTAT | 540 |
| GACTGTGGAC AAGGGAGATA ACGTGAACAT ATCTTTCAAA AAGGTATTGA TTAAAGAAGA | 600 |
| AGATGCAGTG ATTTACAAAA ATGGTTCCTT CATCCATTCA GTGCCCCGGC ATGAAGTACC | 660 |
| TGATATTCTA GAAGTACACC TGCCTCATGC TCAGCCCCAG GATGCTGGAG TGTACTCGGC | 720 |
| CAGGTATATA GGAGGAAACC TCTTCACCTC GGCCTTCACC AGGCTGATAG TCCGGAGATG | 780 |
| TGAAGCCCAG AAGTGGGGAC CTGAATGCAA CCATCTCTGT ACTGCTTGTA TGAACAATGG | 840 |
| TGTCTGCCAT GAAGATACTG GAGAATGCAT TTGCCCTCCT GGGTTTATGG GAAGGACGTG | 900 |
| TGAGAAGGCT TGTGAACTGC ACACGTTTGG CAGAACTTGT AAAGAAAGGT GCASTGGACA | 960 |
| AGAGGGATGC AAGTCTTATG TGTTCTGTCT CCCTGACCCC TATGGGTGTT CCTGTGCCAC | 1020 |
| AGGCTGGAAG GGTCTGCAGT GCAATGAAGC ATGCCACCCT GGTTTTTACG GGCCAGATTG | 1080 |
| TAAGCTTAGG TGCAGCTGCA ACAATGGGGA GATGTGTGAT CGCTTCCAAG GATGTCTCTG | 1140 |
| CTCTCCAGGA TGGCAGGGGC TCCAGTGTGA GAGAGAAGGC ATACCGAGGA TGACCCCAAA | 1200 |
| GATAGTGGAT TTGCCAGATC ATATAGAAGT AAACAGTGGT AAATTTAATC CCATTTGCAA | 1260 |
| AGCTTCTGGC TGGCCGCTAC CTACTAATGA AGAAATGACC CTGGTGAAGC CGGATGGGAC | 1320 |
| AGTGCTCCAT CCAAAAGACT TTAACCATAC GGATCATTTC TCAGTAGCCA TATTCACCAT | 1380 |
| CCACCGGATC CTCCCCCCTG ACTCAGGAGT TTGGGTCTGC AGTGTGAACA CAGTGGCTGG | 1440 |
| GATGGTGGAA AAGCCCTTCA ACATTTCTGT TAAAGTTCTT CCAAAGCCCC TGAATGCCCC | 1500 |
| AAACGTGATT GACACTGGAC ATAACTTTGC TGTCATCAAC ATCAGCTCTG AGCCTTACTT | 1560 |
| TGGGGATGGA CCAATCAAAT CCAAGAAGCT TCTATACAAA CCCGTTAATC ACTATGAGGC | 1620 |
| TTGGCAACAT ATTCAAGTGA CAAATGAGAT TGTTACACTC AACTATTTGG AACCTCGGAC | 1680 |
| AGAATATGAA CTCTGTGTGC AACTGGTCCG TCGTGGAGAG GGTGGGGAAG GGCATCCTGG | 1740 |
| ACCTGTGAGA CGCTTCACAA CAGCTTCTAT CGGACTCCCT CCTCCAAGAG GTCTAAATCT | 1800 |
| CCTGCCTAAA AGTCAGACCA CTCTAAATTT GACCTGGCAA CCAATATTTC CAAGCTCGGA | 1860 |
| AGATGACTTT TATGTTGAAG TGGAGAGAAG GTCTGTGCAA AAAAGTGATC AGCAGAATAT | 1920 |
| TAAAGTTCCA GGCAACTTGA CTTCGGTGCT ACTTAACAAC TTACATCCCA GGGAGCAGTA | 1980 |
| CGTGGTCCGA GCTAGAGTCA ACACCAAGGC CCAGGGGGAA TGGAGTGAAG ATCTCACTGC | 2040 |
| TTGGACCCTT AGTGACATTC TTCCTCCTCA ACCAGAAAAC ATCAAGATTT CCAACATTAC | 2100 |
| ACACTCCTCG GCTGTGATTT CTTGGACAAT ATTGGATGGC TATTCTATTT CTTCTATTAC | 2160 |
| TATCCGTTAC AAGGTTCAAG GCAAGAATGA AGACCAGCAC GTTGATGTGA AGATAAAGAA | 2220 |
| TGCCACCATC ATTCAGTATC AGCTCAAGGG CCTAGAGCCT GAAACAGCAT ACCAGGTGGA | 2280 |
| CATTTTTGCA GAGAACAACA TAGGGTCAAG CAACCCAGCC TTTTCTCATG AACTGGTGAC | 2340 |
| CCTCCCAGAA TCTCAAGCAC CAGCGGACCT CGGAGGGGGG AAGATGCTGC TTATAGCCAT | 2400 |
| CCTTGGCTCT GCTGGAATGA CCTGCCTGAC TGTGCTGTTG GCCTTTCTGA TCATATTGCA | 2460 |
| ATTGAAGAGG GCAAATGTGC AAAGGAGAAT GGCCCAAGCC TTCCAAAACG TGAGGGAAGA | 2520 |
| ACCAGCTGTG CAGTTCAACT CAGGGACTCT GGCCCTAAAC AGGAAGGTCA AAAACAACCC | 2580 |
| AGATCCTACA ATTTATCCAG TGCTTGACTG GAATGACATC AAATTTCAAG ATGTGATTGG | 2640 |
| GGAGGGCAAT TTTGGCCAAG TTCTTAAGGC GCGCATCAAG AAGGATGGGT TACGGATGGA | 2700 |
| TGCTGCCATC AAAAGAATGA AAGAATATGC CTCCAAAGAT GATCACAGGG ACTTTGCAGG | 2760 |
| AGAACTGGAA GTTCTTTGTA AACTTGGACA CCATCCAAAC ATCATCAATC TCTTAGGAGC | 2820 |
| ATGTGAACAT CGAGGCTACT TGTACCTGGC CATTGAGTAC GCGCCCCATG GAAACCTTCT | 2880 |
| GGACTTCCTT CGCAAGAGCC GTGTGCTGGA GACGGACCCA GCATTTGCCA TTGCCAATAG | 2940 |
| CACCGCGTCC ACACTGTCCT CCCAGCAGCT CCTTCACTTC GCTGCCGACG TGGCCCGGGG | 3000 |
| CATGGACTAC TTGAGCCAAA AACAGTTTAT CCACAGGGAT CTGGCTGCCA GAAACATTTT | 3060 |
| AGTTGGTGAA AACTATGTGG CAAAAATAGC AGATTTTGGA TTGTCCCGAG GTCAAGAGGT | 3120 |
| GTACGTGAAA AAGACAATGG CAAGGCTCCC AGTGCGGTGG ATGGCCATCG AGTCACTGAA | 3180 |
| TTACAGTGTG TACACAACCA ACAGTGATGT ATGGTCCTAT GGTGTGTTAC TATGGGAGAT | 3240 |
| TGTTAGCTTA GGAGGCACAC CCTACTGCGG GATGACTTGT GCAGAACTCT ACGACAAGCT | 3300 |
| GCCCCAGGGC TACAGACTGG AGAAGCCCCT GAACTGTGAT GATGAGGTGT ATGATCTAAT | 3360 |
| GAGACAATGC TGGCGGGAGA AGCCTTATCA GAGGCCATCA TTTGCCCAGA TATTGGTGTC | 3420 |
| CTTAAACAGA ATGTTAGAGG AGCGAAAGAC CTACGTGAAT ACCACGCTTT ATGAGAAGTT | 3480 |
| TACTTATGCA GGAATTGACT GTTCTGCTGA AGAAGCGGCC TAGGACAGAA CATCTGTATA | 3540 |
| CCCTCTGTTT CCCTTTCACT GCCATGGGAG ACCCTTGACA ACTGCTGAGA AAACATGCCT | 3600 |
| CTGCCAAAGG ATGTGATATA TAAGTGTACA TATGTGCTGG AATTCTAACA AGTCATAGGT | 3660 |
| TAATATTTAA GACACTGAAA AATCTAAGTG ATATAAATCA GATTCTTCTC TCTCATTTTA | 3720 |
| TCCCTCACCT GTAGCATGCC AGTCCCGTTT CATTTAGTCA TGTGACCACT CTGTCTTGTG | 3780 |
| TTTCCACAGC CTGCAAGTTC AGTCCAGGAT GCTAACATCT AAAAATAGAC TTAAATCTCA | 3840 |
| TTGCTTACAA GCCTAAGAAT CTTTAGAGAA GTATACATAA GTTTAGGATA AAATAATGGG | 3900 |
| ATTTTCTTTT CTTTTCTCTG GTAATATTGA CTTGTATATT TTAAGAAATA ACAGAAAGCC | 3960 |
| TGGGTGACAT TTGGGAGACA TGTGACATTT ATATATTGAA TTAATATCCC TACATGTATT | 4020 |
| GCACATTGTA AAAAGTTTTA GTTTTGATGA GTTGTGAGTT TACCTTGTAT ACTGTAGGCA | 4080 |
| CACTTTGCAC TGATATATCA TGAGTGAATA AATGTCTTGC CTACTCAAAA AAAAAAAA | |

Seq ID No: 147 Protein sequence:
Protein Accession #: NP_000450.1

| | |
|---|---|
| 1 11 21 31 41 51 | |
| \| \| \| \| \| \| | |
| MDSLASLVLC CVSLLLSGTV EGANDLILIN SLPLVSDAET SLTCIASCWR PHEPITIGRD | 60 |
| FEALMNQhQD PLEVTQDVTR EWAKKVVWKR EKASKINGAY FCEGRVRGEA IRIRTMKMRQ | 120 |
| QASFLPATLT MTVDKGDNVN ISFKKVLIKE EDAVIYKNGS FIHSVPRHEV PDILEVHLPH | 180 |
| AQPQDAGVYS ARYIGGNLFT SAFTRLIVRR CEAQKWGPEC NHLCTACMNN GVCHEDTGEC | 240 |
| ICPPGFMGRT CEKACELHTF GRTCKERCSG QEGCKSYVFC LPDPYGCSCA TGWKGLQCNE | 300 |
| ACHPGFYGPD CKLRCSCNNG EMCDRFQGCL CSPGWQGLQC EREGIPRMTP KIVDLPDHIE | 360 |
| VNSGKFNPIC KASGWPLPTN EEMTLVKPDG TVLHPKDFNH TDHFSVAIFT IHRILPPDSG | 420 |
| VWVCSVNTVA GMVEKPFNIS VKVLPKPLNA PNVIDTGHNF AVINISSEPY FGDGPIKSKK | 480 |
| LLYKPVNHYE AWQHIQVTNE IVTLNYLEPR TEYELCVQLV RRGEGGEGHP GPVRRFTTAS | 540 |
| IGLPPPRGLN LLPKSQTTLN LTWQPIFPSS EDDFYVEVER RSVQKSDQQN IKVPGNLTSV | 600 |
| LLNNLHPREQ YVVRARVNTK AQGEWSEDLT AWTLSDILPP QPENIKISNI THSSAVISWT | 660 |
| ILDGYSISSI TIRYKVQGKN EUQHYDYKIK NATIIQYQLK GLEPETAYQV DIFAENNIGS | 720 |
| SNPAFSHELV TLPESQAPAD LGGGKMLLIA ILGSAGMTCL TVLLAFLIIL QLKRANVQRR | 780 |
| MAQAFQNVRE EPAVQFNSGT LALNRKVKNN PDPTIYPVLD WNDIKFQDVI GECNFGQVLK | 840 |
| ARIKKDGLRM DAAIKRMKEY ASKDDHRDFA GELEVLCKLG HHPNIINLLC ACEHRGYLYL | 900 |
| AIEYAPHGNL LDFLRKSRVL ETDPAFAIAN STASTLSSQQ LLHFAADVAR GMDYLSQKQF | 960 |
| IHRDLAARNI LVGENYVAKI ADFGLSRGQE VYVKKTMGRL PVRWMAIESL NYSVYTTNSD | 1020 |
| VWSYGVLLWE IVSLGGTPYC GMTCAELYEK LPQCYRLEKP LNCDDEVYDL MRQCWREKPY | 1080 |
| ERPSFAQILV SLNRMLEERK TYVNTTLYEK FTYAGIDCSA EEAA | |

Seq ID NO: 148 DNA sequence:
Nucleic Acid Accession #: NM_000552.2
Coding sequence: 311-8752 (underlined sequences correspond to start and stop codons)

| | |
|---|---|
| 1 11 21 31 41 51 | |
| \| \| \| \| \| \| | |
| AGCTCACAGC TATTGTGGTG GGAAAGGGAG GGTGGTTGGT GGATGTCACA GCTTGGGCTT | 60 |
| TATCTCCCCC AGCAGTGGGG ACTCCACAGC CCCTGGGCTA CATAACAGCA AGACAGTCCG | 120 |
| GAGCTGTAGC AGACCTGATT GAGCCTTTGC AGCAGCTGAG AGCATGGCCT AGGGTGCGCG | 180 |
| GCACCATTGT CCAGCAGCTG AGTTTCCCAG GGACCTTGGA GATAGCCGCA GCCCTCATTT | 240 |
| GCAGGGGAAG GCACCATTGT CCAGCAGCTG AGTTTCCCAG GGACCTTGGA GATAGCCGCA | 300 |
| GCCCTCATTT ATGATTCCTG CCAGATTTGC CGGGGTGCTG CTTGCTCTGG CCCTCATTTT | 360 |
| GCCAGGGACC CTTTGTGCAG AAGGAACTCG CGGCAGGTCA TCCACGGCCC GATGCAGCCT | 420 |
| TTTCGGAAGT GACTTCGTCA ACACCTTTGA TGGGAGCATG TACAGCTTTG CGGGATACTG | 480 |
| CAGTTACCTC CTGGCAGGGG GCTGCCAGAA ACGCTCCTTC TCGATTATTG GGGACTTCCA | 540 |
| GAATGGCAAG AGAGTGAGCC TCTCCGTGTA TCTTGGGGAA TTTTTTGACA TCCATTTGTT | 600 |
| TGTCAATGGT ACCGTGACAC AGGGGGACCA AAGAGTCTCC ATGCCCTATG CCTCCAAAGG | 660 |
| GCTGTATCTA GAAACTGAGG CTGGGTACTA CAAGCTGTCC GGTGAGGCCT ATGGCTTTGT | 720 |
| GGCCAGGATC GATGGCAGCG GCAACTTTCA AGTCCTGCTG TCAGACAGAT ACTTCAACAA | 780 |
| GACCTGCGGG CTGTGTGGCA ACTTTAACAT CTTTGCTGAA GATGACTTTA TGACCCAAGA | 840 |
| AGGGACCTTG ACCTCGGACC CTTATGACTT TGCCAACTCA TGGGCTCTGA GCAGTGGAGA | 900 |
| ACAGTGGTGT GAACGGGCAT CTCCTCCCAG CAGCTCATGC AACATCTCCT CTGGGGAAAT | 960 |
| GCAGAAGGGC CTGTGGGAGC AGTGCCAGCT TCTGAAGAGC ACCTCGGTGT TTGCCCGCTG | 1020 |
| CCACCCTCTG GTGGACCCCG AGCCTTTTGT GGCCCTGTGT GAGAAGACTT TGTGTGAGTG | 1080 |
| TGCTGGGGGG CTGGAGTGCG CCTGCCCTGC CCTCCTGGAG TACGCCCGGA CCTGTGCCCA | 1140 |
| GGAGGGAATG GTGCTGTACG GCTGGACCGA CCACAGCGCG TGCAGCCCAG TGTGCCCTGC | 1200 |
| TGGTATGGAG TATAGGCAGT GTGTGTCCCC TTGCGCCAGG ACCTGCCAGA GCCTGCACAT | 1260 |
| CAATGAAATG TGTCAGGAGC GATGCGTGGA TGGCTGCAGC TGCCCTGAGG GACAGCTCCT | 1320 |
| GGATGAAGGC CTCTGCGTGG AGAGCACCGA GTGTCCCTGC GTGCATTCCG GAAAGCGCTA | 1380 |
| CCCTCCCGGC ACCTCCCTCT CTCGAGACTG CAACACCTGC ATTTGCCGAA ACAGCCAGTG | 1440 |
| GATCTGCAGC AATGAAGAAT GTCCAGGGGA GTGCCTTGTC ACTGGTCAAT CCCACTTCAA | 1500 |
| GAGCTTTGAC AACACATACT TCACCTTCAG TGGGATCTGC CAGTACCTGC TGGCCCGGGA | 1560 |
| TTGCCAGGAC CACTCCTTCT CCATTGTCAT TGAGACTGTC CAGTGTGCTG ATGACCGCGA | 1620 |
| CGCTGTGTGC ACCCGCTCCG TCACCGTCCG GCTGCCTGGC CTGCACAACA GCCTTGTGAA | 1680 |
| ACTGAAGCAT GGGGCAGGAG TTGCCATGGA TGGCCAGGAC ATCCAGCTCC CCCTCCTGAA | 1740 |
| AGGTGACCTC CGCATCCAGC ATACAGTGAC GGCCTCCGTG CGCCTCAGCT ACGGGGAGGA | 1800 |
| CCTGCAGATG GACTGGGATG GCCGCGGGAG GCTGCTGGTG AAGCTGTCCC CCGTCTACGC | 1860 |
| CGGGAAGACC TGCGGCCTGT GTGGGAATTA CAATGGCAAC CAGGGCGACG ACTTCCTTAC | 1920 |
| CCCCTCTGGG CTGGCAGAGC CCCGGGTGGA GGACTTCGGG AACGCCTGGA AGCTGCACGG | 1980 |
| GGACTGCCAG GACCTGCAGA ACCAGCACAG CGATCCCTGC GCCCTCAACC CGCGCATGAC | 2040 |
| CAGGTTCTCC GAGGAGGCGT GCGCGGTCCT GACGTCCCCC ACATTCGAGG CCTGCCATCG | 2100 |
| TGCCGTCAGC CCGCTGCCCT ACCTGCGGAA CTGCCGCTAC GACGTGTGCT CCTGCTCGGA | 2160 |
| CGGCCGCGAG TGCCTGTGCG GCGCCCTGGC CAGCTATGCC GCGGCCTGCG CGGGGAGAGG | 2220 |
| CGTGCGCGTC GCGTGGCGCG AGCCAGGCCG CTGTGAGCTG AACTGCCCGA AAGGCCAGGT | 2280 |
| GTACCTGCAG TGCGGGACCC CCTGCAACCT GACCTGCCGC TCTCTCTCTT ACCCGGATGA | 2340 |
| GGAATGCAAT GAGGCCTGCC TGGAGGGCTG CTTCTGCCCC CCAGGGCTCT ACATGGATGA | 2400 |
| GAGGGGGGAC TGCGTGCCCA AGGCCCAGTG CCCCTGTTAC TATGACGGTG AGATCTTCCA | 2460 |
| GCCAGAAGAC ATCTTCTCAG ACCATCACAC CATGTGCTAC TGTGAGGATG GCTTCATGCA | 2520 |
| CTGTACCATG AGTGGAGTCC CCGGAAGCTT GCTGCCTGAC GCTGTCCTCA GCAGTCCCCT | 2580 |
| GTCTCATCGC AGCAAAAGGA GCCTATCCTG TCGGCCCCCC ATGGTCAAGC TGGTGTGTCC | 2640 |
| CGCTGACAAC CTGCGGGCTG AAGGGCTCGA GTGTACCAAA ACGTGCCAGA ACTATGACCT | 2700 |
| GGAGTGCATG AGCATGGGCT GTCTCTCTGG CTGCCTCTGC CCCCCGGGCA TGGTCCGGCA | 2760 |
| GGAGTGCATG AGCATCGGCT GTGTCTCTGG CTGCCTCTGC CCCCCGGGCA TGGTCCGGCA | 2760 |
| TGAGAACAGA TGTGTGGCCC TGGAAAGGTG TCCCTGCTTC CATCAGGGCA AGGAGTATGC | 2820 |
| CCCTGGAGAA ACAGTGAAGA TTGGCTGCAA CACTTGTGTC TGTCGGGACC GGAAGTGGAA | 2880 |
| CTGCACAGAC CATGTGTGTG ATGCCACGTG CTCCACGATC GGCATGGCCC ACTACCTCAC | 2940 |
| CTTCGACGGG CTCAAATACC TGTTCCCCGG GGAGTGCCAG TACGTTCTGG TGCAGGATTA | 3000 |
| CTGCGGCAGT AACCCTGGGA CCTTTCGGAT CCTAGTGGGG AATAAGGGAT GCAGCCACCC | 3060 |
| CTCAGTGAAA TGCAAGAAAC GGGTCACCAT CCTGGTGGAG GGAGGAGAGA TTGAGCTGTT | 3120 |
| TGACGGGGAG GTGAATGTGA AGAGGCCCAT GAAGGATGAG ACTCACTTTG AGGTGGTGGA | 3180 |
| GTCTGGCCGG TACATCATTC TGCTGCTGGG CAAAGCCCTC TCCGTGGTCT GGGACCGCCA | 3240 |
| CCTGAGCATC TCCGTGGTCC TGAAGCAGAC ATACCAGGAG AAAGTGTGTG GCCTGTGTGG | 3300 |
| GAATTTTGAT GGCATCCAGA ACAATGACCT CACCAGCAGC AACCTCCAAG TGGAGGAAGA | 3360 |
| CCCTGTGGAC TTTGGGAACT CCTGGAAAGT GAGCTCGCAG TGTGCTGACA CCAGAAAAGT | 3420 |
| GCCTCTGGAC TCATCCCCTG CCACCTGCCA TAACAACATC ATGAAGCAGA CGATGGTGGA | 3480 |
| TTCCTCCTGT AGAATCCTTA CCAGTGACGT CTTCCAGGAC TGCAACAAGC TGGTGGACCC | 3540 |
| CGAGCCATAT CTGGATGTCT GCATTTACGA CACCTGCTCC TGTGAGTCCA TTGGGGACTG | 3600 |
| CGCCTGCTTC TGCGACACCA TTGCTGCCTA TGCCCACGTG TGTGCCCAGC ATGGCAAGGT | 3660 |
| GGTGACCTGG AGGACGGCCA CATTGTGCCC CCAGAGCTGC GAGGAGAGGA ATCTCCGGGA | 3720 |
| GAACGGGTAT GAGTGTGAGT GGCGCTATAA CAGCTGTGCA CCTGCCTGTC AAGTCACGTG | 3780 |
| TCAGCACCCT GAGCCACTGG CCTGCCCTGT GCAGTGTGTG GAGGGCTGCC ATGCCCACTG | 3840 |
| CCCTCCAGGG AAAATCCTGG ATGAGCTTTT GCAGACCTGC GTTGACCCTG AAGACTGTCC | 3900 |
| AGTGTGTGAG GTGGCTGGCC GGCGTTTTGC CTCAGGAAAG AAAGTCACCT TGAATCCCAG | 3960 |
| TGACCCTGAG CACTGCCAGA TTTGCCACTG TGATGTTGTC AACCTCACCT GTGAAGCCTG | 4020 |
| CCAGGAGCCG GGAGGCCTGG TGGTGCCTCC CACAGATGCC CCGGTGAGCC CCACCACTCT | 4080 |
| GTATGTGGAG GACATCTCGG AACCGCCGTT GCACGATTTC TACTGCAGCA GGCTACTGGA | 4140 |
| CCTGGTCTTC CTGCTGGATG GCTCCTCCAG GCTGTCCGAG GCTGAGTTTG AAGTGCTGAA | 4200 |
| GGCCTTTGTG GTGGACATGA TGGAGCGGCT GCGCATCTCC CAGAAGTGGG TCCGCGTGGC | 4260 |
| CGTGGTGGAG TACCACGACG GCTCCCACGC CTACATCGGG CTCAAGGACC GGAAGCGACC | 4320 |
| GTCAGAGCTG CGGCGCATTG CCAGCCAGGT GAAGTATGCG GGCAGCCAGG TGGCCTCCAC | 4380 |
| CAGCGAGGTC TTGAAATACA CACTGTTCCA AATCTTCAGC AAGATCGACC GCCCTGAAGC | 4440 |
| CTCCCGCATC GCCCTGCTCC TGATGGCCAG CCAGGAGCCC CAACGGATGT CCCGGAACTT | 4500 |
| TGTCCGCTAC GTCCAGGGCC TGAAGAAGAA GAAGGTCATT GTGATCCCGG TGGGCATTGG | 4560 |
| GCCCCATGCC AACCTCAAGC AGATCCGCCT CATCGAGAAG CAGGCCCCTG AGAACAAGGC | 4620 |
| CTTCGTGCTG AGCAGTGTGG ATGAGCTGGA GCAGCAAAGG GACGAGATCG TTAGCTACCT | 4680 |
| CTGTGACCTT GCCCCTGAAG CCCCTCCTCC TACTCTGCCC CCCCACATGG CACAAGTCAC | 4740 |
| TGTGGGCCCG GGGCTCTTGG GGGTTTCGAC CCTGGGGCCC AAGAGGAACT CCATGGTTCT | 4800 |
| GGATGTGGCG TTCGTCCTGG AAGGATCGGA CAAAATTGGT GAAGCCGACT TCAACAGGAG | 4860 |
| CAAGGAGTTC ATGGAGGAGG TGATTCAGCG GATGGATGTG GGCCAGGACA GCATCCACGT | 4920 |
| CACGGTGCTG CAGTACTCCT ACATGGTGAC CGTGGAGTAC CCCTTCAGCG AGGCACAGTC | 4980 |
| CAAAGGGGAC ATCCTGCAGC GGGTGCGAGA GATCCGCTAC CAGGGCGGCA ACAGGACCAA | 5040 |
| CACTGGGCTG GCCCTGCGGT ACCTCTCTGA CCACAGCTTC TTGGTCAGCC AGGGTGACCG | 5100 |
| GGAGCAGGCG CCCAACCTGG TCTACATGGT CACCGGAAAT CCTGCCTCTG ATGAGATCAA | 5160 |
| GAGGCTGCCT GGAGACATCC AGGTGGTGCC CATTGGAGTG GGCCCTAATG CCAACGTGCA | 5220 |
| GGAGCTGGAG AGGATTGGCT GGCCCAATGC CCCTATCCTC ATCCAGGACT TTGAGACGCT | 5280 |
| CCCCCGAGAG GCTCCTGACC TGGTGCTGCA GAGGTGCTGC TCCGGAGAGG GGCTGCAGAT | 5340 |
| CCCCACCCTC TCCCCTGCAC CTGACTGCAG CCAGCCCCTG GACGTGATCC TTCTCCTGGA | 5400 |
| TGGCTCCTCC AGTTTCCCAG CTTCTTATTT TGATGAAATG AAGAGTTTCG CCAAGGCTTT | 5460 |
| CATTTCAAAA GCCAATATAG GGCCTCGTCT CACTCAGGTG TCAGTGCTGC AGTATGGAAG | 5520 |
| CATCACCACC ATTGACGTGC CATGGAACGT GGTCCCGGAG AAAGCCCATT TGCTGAGCCT | 5580 |
| TGTGGACGTC ATGCAGCGGG AGGGAGGCCC CAGCCAAATC GGGGATGCCT TGGGCTTTGC | 5640 |
| TGTGCGATAC TTGACTTCAG AAATGCATGG TGCCAGGCCG GGAGCCTCAA AGGCGGTGGT | 5700 |
| CATCCTGGTC ACGGACGTCT CTGTGGATTC AGTGGATGCA GCAGCTGATG CCGCCAGGTC | 5760 |
| CAACAGAGTG ACAGTGTTCC CTATTGGAAT TGGAGATCGC TACGATGCAG CCCAGCTACG | 5820 |
| GATCTTGGCA GGCCCAGCAG GCGACTCCAA CGTGGTGAAG CTCCAGCGAA TCGAAGACCT | 5880 |
| CCCTACCATG GTCACCTTGG GCAATTCCTT CCTCCACAAA CTGTGCTCTG GATTTGTTAG | 5940 |
| GATTTGCATG GATGAGGATG GGAATGAGAA GAGGCCCGGG GACGTCTGGA CCTTGCCAGA | 6000 |
| CCAGTGCCAC ACCGTGACTT GCCAGCCAGA TGGCCAGACC TTGCTGAAGA GTCATCGGGT | 6060 |
| CAACTGTGAC CGGGGGCTGA GGCCTTCGTG CCCTAACAGC CAGTCCCCTG TTAAAGTGCA | 6120 |
| AGAGACCTGT GGCTGCCGCT GGACCTGCCC CTGCGTGTGC ACAGGCAGCT CCACTCGGCA | 6180 |
| CATCGTGACC TTTGATGGGC AGAATTTCAA GCTGACTGGC AGCTGTTCTT ATGTCCTATT | 6240 |
| TCAAAACAAG GAGCAGGACC TGGAGGTGAT TCTCCATAAT GGTGCCTGCA GCCCTGGAGC | 6300 |
| AAGGCAGGGC TGCATGAAAT CCATCGAGGT GAAGCACAGT GCCCTCTCCG TCGAGCTGCA | 6360 |
| CAGTGACATG GAGGTGACGG TGAATGGGAG ACTGGTCTCT GTTCCTTACG TGGGTGGGAA | 6420 |
| CATGGAAGTC AACGTTTATG GTGCCATCAT GCATGAGGTC AGATTCAATC ACCTTGGTCA | 6480 |
| CATCTTCACA TTCACTCCAC AAAACAATGA GTTCCAACTG CAGCTCAGCC CCAAGACTTT | 6540 |
| TGCTTCAAAG ACGTATCGTC TGTGTGGGAT GTGTGATGAG AACGGAGCCA ATGACTTCAT | 6600 |
| GCTGAGGGAT GGCACAGTCA CCACAGACTG GAAAACACTT GTTCAGGAAT GGACTGTGCA | 6660 |
| GCGGCCAGGG CAGACGTGCC AGCCCATCCT GGAGGAGCAG TGTCTTGTCC CCCACAGCTC | 6720 |
| CCACTGCCAG GTCCTCCTCT TACCACTGTT TGCTGAATGC CACAAGGTCC TGGCTCCAGC | 6780 |
| CACATTCTAT GCCATCTGCC AGCAGGACAG TTGCCACCAG GAGCAAGTGT GTGAGGTGAT | 6840 |
| CGCCTCTTAT GCCCACCTCT GTCGGACCAA CGGGGTCTGC GTTGACTGGA GGACACCTGA | 6900 |
| TTTCTGTGCT ATGTCATGCC CACCATCTCT GGTCTACAAC CACTGTGAGC ATGGCTGTCC | 6960 |
| CCGGCACTGT GATGGCAACG TGAGCTCCTG TGGGGACCAT CCCTCCGAAG GCTGTTTCTG | 7020 |
| CCCTCCAGAT AAAGTCATGT TGGAAGGCAG CTGTGTCCCT GAAGAGGCCT GCACTCAGTG | 7080 |
| CATTGGTGAG GATGGAGTCC AGCACCAGTT CCTGGAAGCC TGGCTCCCGG ACCACCAGCC | 7140 |
| CTGTCAGATC TGCACATGCC TCAGCGGGCG GAAGGTCAAC TGCACAACGC AGCCCTGCCC | 7200 |
| CACGGCCAAA CCTCCCACGT GTGGCCTCTG TGAAGTAGCC CGCCTCCGCC AGAATGCAGA | 7260 |
| CCAGTGCTGC CCCGAGTATG AGTGTGTGTG TGACCCAGTG AGCTGTGACC TGCCCCCAGT | 7320 |
| GCCTCACTGT GAACGTGGCC TCCAGCCCAC ACTGACCAAC CCTGGCGAGT GCAGACCCAA | 7380 |
| CTTCACCTGC GCCTGCAGGA AGGAGGAGTG CAAAAGAGTG TCCCCACCCT CCTGCCCCCC | 7440 |
| GCACCGTTTG CCCACCCTTC GGAAGACCCA GTGCTGTGAT GAGTATGAGT GTGCCTGCAA | 7500 |
| CTGTGTCAAC TCCACAGTGA GCTGTCCCCT TGGGTACTTG GCCTCAACCG CCACCAATGA | 7560 |
| CTGTGGCTGT ACCACAACCA CCTGCCTTCC CGACAAGGTG TGTGTCCACC GAAGCACCAT | 7620 |
| CTACCCTGTG GGCCAGTTCT GGGAGGAGGG CTGCGATGTG TGCACCTGCA CCGACATGGA | 7680 |
| GGATGGCCTG ATGGGCCTCC GCGTGGCCCA GTGCTCCCAG AAGCCCTGTG AGGACAGCTG | 7740 |
| TCGGTCGGGC TTCACTTACG TTCTGCATGA AGGCGAGTGC TGTGGAAGGT GCCTGCCATC | 7800 |
| TGCCTGTGAG GTGGTGACTG GCTCACCGCG GGGGGACTCC CAGTCTTCCT GGAAGAGTGT | 7860 |
| CGGCTCCCAG TGGGCCTCCC CGGAGAACCC CTGCCTCATC AATGAGTGTG TCCGAGTGAA | 7920 |
| GGAGGAGGTC TTTATACAAC AAAGGAACGT CTCCTGCCCC CAGCTGGAGG TCCCTGTCTG | 7980 |
| CCCCTCGGGC TTTCAGCTGA GCTGTAAGAC CTCAGCGTGC TGCCCAAGCT GTCGCTGTGA | 8040 |
| GCGCATGGAG GCCTGCATGC TCAATGGCAC TGTCATTGGG CCCGGGAAGA CTGTGATGAT | 8100 |
| CGATGTGTGC ACGACCTGCC GCTGCATGGT GCAGGTGGGG GTCATCTCTG GATTCAAGCT | 8160 |
| GGAGTGCAGG AAGACCACCT GCAACCCCTG CCCCCTGGGT TACAAGGAAG AAAATAACAC | 8220 |
| AGGTGAATGT TGTGGGAGAT GTTTGCCTAC GGCTTGCACC ATTCAGCTAA GAGGAGGACA | 8280 |
| GATCATGACA CTGAAGCGTG ATGAGACGCT CCAGGATGGC TGTGATACTC ACTTCTGCAA | 8340 |
| GGTCAATGAG AGAGGAGAGT ACTTCTGGGA GAAGAGGGTC ACAGGCTGCC CACCCTTTGA | 8400 |
| TGAACACAAG TGTCTGGCTG AGGGAGGTAA AATTATGAAA ATTCCAGGCA CCTGCTGTGA | 8460 |
| CACATGTGAG GAGCCTGAGT GCAACGACAT CACTGCCAGG CTGCAGTATG TCAAGGTGGG | 8520 |
| AAGCTGTAAG TCTGAAGTAG AGGTGGATAT CCACTACTGC CAGGGCAAAT GTGCCAGCAA | 8580 |
| AGCCATGTAC TCCATTGACA TCAACGATGT GCAGGACCAG TGCTCCTGCT GCTCTCCGAC | 8640 |
| ACGGACGGAG CCCATGCAGG TGGCCCTGCA CTGCACCAAT GGCTCTGTTG TGTACCATGA | 8700 |
| GGTTCTCAAT GCCATGGAGT GCAAATGCTC CCCCAGGAAG TGCAGCAAGT GAGGCTGCTG | 8760 |
| CAGCTGCATG GGTGCCTGCT GCTGCCTGCC TTGGCCTGAT GGCCAGGCCA GAGTGCTGCC | 8820 |
| AGTCCTCTGC ATGTTCTGCT CTTGTGCCCT TCTGAGCCCA CAATAAAGGC TGAGCTCTTA | 8880 |
| TCTTGCTGCA TGTTCTGCTC TTGTGCCCTT CTGAGCCCAC AAT | |

Seq ID No: 149 Protein sequence:
Protein Accession #: NP_000543.1

| | |
|---|---|
| 1 11 21 31 41 51 | |
| \| \| \| \| \| \| | |
| MIPARFAGVL LALALILPGT LCAEGTRGRS STARCSLFGS DFVNTFDGSM YSFAGYCSYL | 60 |
| LAGGCQKRSF SIIGDFQNGK RVSLSVYLGE FFDIHLFVNG TVTQGDQRVS MPYASKGLYL | 120 |
| ETEAGYYKLS GEAYGFVARI DGSGNFQVLL SDRYFNKTCG LCGNFNIFAE DDFMTQEGTL | 180 |
| TSDPYDFANS WALSSGEQWC ERASPPSSSC NISSGEMQKG LWEQCQLLKS TSVFARCHPL | 240 |
| VDPEPFVALC EKTLCECAGG LECACPALLE YARTCAQEGM VLYGWTDHSA CSPVCPAGME | 300 |
| YPQCVSPCAR TCQSLHINEM CQERCVDGCS CPEGQLLDEG LCVESTECPC VHSGKRYPPG | 360 |
| TSLSRDCNTC TCRNSQWTCS NEECPGECLV TGQSHFKSFD NRYFTFSGIC QYLLARDCQD | 420 |
| HSFSIVIETV QCADDRDAVC TRSVTVRLPG LHNSLVKLKH GAGVANDGQD IQLPLLKGDL | 480 |
| RIQHTVTASV RLSYGEDLQM DWDGRGRLLV KLSPVYAGKT CGLCGNYNGN QGDDFLTPSG | 540 |
| LAEPRVEDFG NAWKLHGDCQ DLQKQHSDPC ALNPRMTRFS EEACAVLTSP TFEACHRAVS | 600 |
| PLPYLRNCRY DVCSCSDGRE CLCGALASYA AACAGRGVRV AWREPGRCEL NCPKGQVYLQ | 660 |
| CGTPCNLTCR SLSYPDEECN EACLEGCFCP PGLYMDERGD CVPKAQCPCY YDGEIFQFED | 720 |
| IFSDHHTMCY CEDGFMHCTM SGVPGSLLPD AVLSSPLSHR SKRSLSCRPP MVKLVCPADN | 780 |
| LRAEGLECTK TCQNYDLECM SMGCVSGCLC PPGMVRHENR CVALERCPCF HQGKEYAPGE | 840 |
| TVKIGCNTCV CRDRKWNCTD HVCDATCSTI GMAHYLTFDG LKYLFPGECQ YVLVQDYCGS | 900 |
| NPGTFRILVG NKGCSHPSVK CKKRVTILVE GGEIELFDGE VNVKRPMKDE THFEVVESGR | 960 |
| YIILLLGKAL SVVWDRHLSI SVVLKQTYQE KVCGLCGNFD GIQNNDLTSS NLQVEEDPVD | 1020 |
| FGNSWKVSSQ CADTRKVPLD SSPATCHNNI MKQTMVDSSC RILTSDVFQD CNKLVDPEPY | 1080 |
| LDVCIYDTCS CESIGDCACF CDTIAAYAHV CAQHGKVVTW RTATLCPQSC EERNLRSNGY | 1140 |
| ECEWRYNSCA PACQVTCQNP EPLACPVQCV EGCHAHCPPG KILDELLQTC VDPEDCPVCE | 1200 |
| VAGRRFASGK KVTLNPSDPE HCQICHCGVV NLTCEACQEP GGLVVPPTDA PVSPTTLYVE | 1260 |
| DISEPPLHDF YCSRLLDLVF LLDGSSRLSE AEFEVLKAFV VDMMERLRIS QKWVRVAVVE | 1320 |
| YHDGSHAYIG LKDRKRPSEL RRIASQVKYA GSQVASTSEV LKYTLFQIFS KIGRPEASRI | 1380 |
| ALLLMASQEP QRMSRNFVRY VQGLKKKKVI VIPVGIGPHA NLKQIRLIEK QAPENKAFVL | 1440 |
| SSVDELEQQR DEIVSYLCDL APEAPPPTLP PHMAQVTVGF GLLGVSTLGP KRNSMVLDVA | 1500 |
| FVLEGSDKIG EADFNRSKEF MEEVIQRMDV GQDSIHVTVL QYSYMVTVEY PFSEAQSKGD | 1560 |
| ILQRVREIRY QGCNRTNTGL ALRYLSDHSF LVSQGDREQA PNLVYMVTGN PASDEIKRLP | 1620 |
| GDIQVVPIGV GPNANVQELE RIGWPNAPIL IQDFETLPRE APDLVLQRCC SGEGLQIPTL | 1680 |
| SPAPDCSQPL DVILLLDGSS SFPASYFDEM KSFAKAFISK ANIGPRLTQV SVLQYGSITT | 1740 |
| IDVPWNVVPE KAHLLSLVDV MQREGGPSQI GDALCFAVRY LTSEMHCARP CASKAVVILV | 1800 |
| TDVSVDSVDA AADAARSNRV TVFPICICDR YDAAQLRIEA GPAGDSNVVK LQRIEDLPTM | 1860 |
| VTLGNSFLHK LCSGFVRICM DEDGNERRPG DVWTLPDQCH TVTCQPDGQT LLKSHRVNCD | 1920 |
| RCLRPSCPNS QSPVKVEETC GCRWTCPCVC TGSSTRHIVT FDGQNFKLTG SCSYVLFQNK | 1980 |
| EQDLEVILHN GACSPGARQG CMKSIEVKHS ALSVELHSDM EVTVNGRLVS VPYVGGNMEV | 2040 |
| NVYCAIMHEV RFNHLCHIFT FTPQNNEFQL QLSPKTFASI TYGLCGICDE NGANDFMLRD | 2100 |
| GTVTTDWKTL VQEWTVQRPG QTCQPILEEQ CLVPDSSHCQ VLLLPLFAEC HKVLAPATFY | 2160 |
| AICQQDSCEQ EQVCEVIASY AHLCRTNGVC VDWRTPDFCA MSCPPSLVYN HCEHGCPRHC | 2220 |
| DGNVSSCGDH PSECCFCPPD KVMLEGSCVP EEACTQCIGE DGVQHQFLEA WVPDHQPCQI | 2280 |
| CTCLSGRKVN CTTQPCPTAK APTCGLCEVA RLRQNADQCC PEYECVCDPV SCDLPPVPHC | 2340 |
| ERGLQPTLTN PGECRPNFTC ACRKEECKRV SPPSCPPHRL PTLRKTQCCD EYECACNCVN | 2400 |
| STVSCPLGYL ASTATNDCCC TTTTCLPDKV CVHRSTIYPV GQFWEEGCDV CTCTDMEDAV | 2460 |
| MCLRVAQCSQ KPCEDSCRSG FTYVLHEGEC CGRCLPSACE VVTGSPRGDS QSSWKSVGSQ | 2520 |
| WASPENPCLI NECVRVKEEV FIQQRNVSCP QLEVPVCPSG FQLSCKTSAC CPSCRCERME | 2580 |
| ACMLNGTVIG PGKTVMIDVC TTCRCMVQVG VISGFKLECR RTTCNPCPLG YKEENNTGEC | 2640 |
| CGRCLPTACT IQLRGGQIMT LKRDETLQDG CDTHFCKVNE RGEYFWEKRV TGCPPFDEHK | 2700 |
| CLAEGGKIMK IPGTCCDTCE EPECNDITAR LQYVKVGSCK SEVEVDIHYC QGECASKAMY | 2760 |
| SIDINDVQDQ CSCCSPTRTE PMQVALHCTN GSVVYHEVLN AMECKCSPRK CSK | |

Seq ID NO: 150 DNA sequence:
Nucleic Acid Accession #: NM_001508.1
Coding sequence: 1-1362 (underlined sequences correspond to start and stop codons)

| | |
|---|---|
| 1 11 21 31 41 51 | |
| \| \| \| \| \| \| | |
| ATGGCTTCAC CCAGCCTCCC GGGCAGTGAC TGCTCCCAAA TCATTGATCA CAGTCATGTC | 60 |
| CCCGAGTTTG AGGTGGCCAC CTGGATCAAA ATCACCCTTA TTCTGGTGTA CCTGATCATC | 120 |
| TTCGTGATGG GCCTTCTGGG GAACAGCGTC ACCATTCGGG TCACCCAGGT GCTGCAGAAG | 180 |
| AAAGGATACT TGCAGAAGGA GGIGACAGAC CACATGGTGA GTTTGGCTTG CTCGGACATC | 240 |
| TTGGTGTTCC TCATCGGCAT GCCCATGCAG TTCTACAGCA TCATCTGGAA TCCCCTGACC | 300 |
| ACGTCCAGCT ACACCCTGTC CTGCAAGCTG CACACTTTCC TCTTCGAGGC CTGCAGCTAC | 360 |
| GCTACGCTGC TGCACGTGCT GACGCTCAGC TTTGAGCGCT ACATCGCCAT CTGTCACCCC | 420 |
| TTCAGGTACA AGGCTGTGTC GGGACCTTGC CAGGTGAAGC TGCTGATTGG CTTCGTCTGG | 480 |
| GTCACCTCCG CCCTGGTGGC ACTGCCCTTG CTGTTTGCCA TGGGTACTGA GTACCCCCTG | 540 |
| GTGAACGTGC CCAGCCACCG GGGTCTCACT TGCAACCGCT CCAGCACCCG CCACCACGAG | 600 |
| CAGCCCGAGA CCTCCAATAT GTCCATCTGT ACCAACCTCT CCAGCCGCTG GACCGTGTTC | 660 |
| CAGTCCAGCA TCTTCGGCGC CTTCGTGGTC TACCTCGTGG TCCTGCTCTC CGTAGCCTTC | 720 |
| ATGTGCTGGA ACATGATGCA GGTGCTCATG AAAAGCCAGA AGGGCTCGCT GGCCGGGGGC | 780 |
| ACGCGGCCTC CGCAGCTGAG GAAGTCCGAG AGCGAAGAGA GCAGGACCGC CAGGAGGCAG | 840 |
| ACCATCATCT TCCTGAGGCT GATTGTTGTG ACATTGGCCG TATGCTGGAT GCCCAACCAG | 900 |
| ATTCGGAGGA TCATGGCTGC GGCCAAACCC AAGCACGACT GGACGAGGTC CTACTTCCGG | 960 |
| GCGTACATGA TCCTCCTCCC CTTCTCGGAG ACGTTTTTCT ACCTCAGCTC GGTCATCAAC | 1020 |
| CCGCTCCTGT ACACGGTGTC CTCGCAGCAG TTTCGGCGGG TGTTCGTGCA GGTGCTGTGC | 1080 |
| TGCCGCCTGT CGCTGCAGCA CGCCAACCAC GAGAAGCGCC TGCGCGTACA TGCGCACTCC | 1140 |
| ACCACCGACA GCGCCCGCTT TGTGCAGCGC CCGTTGCTCT TCGCGTCCCG GCGCCAGTCC | 1200 |
| TCTGCAAGGA GAACTGAGAA GATTTTCTTA AGCACTTTTC AGAGCGAGGC CGAGCCCCAG | 1260 |
| TCTAAGTCCC AGTCATTGAG TCTCGAGTCA CTAGAGCCCA ACTCAGGCGC GAAACCAGCC | 1320 |
| AATTCTGCTG CAGAGAATGG TTTTCAGGAG CATGAAGTTT GA | |

Seq ID No: 151 Protein sequence:
Protein Accession #: NP_001499.1

| | |
|---|---|
| 1 11 21 31 41 51 | |
| \| \| \| \| \| \| | |
| MASPSLPGSD CSQIIDHSHV PEFEVATWIK ITLILVYLIT FVMGLLGNSV TIRVTQVLQK | 60 |
| KGYLQKEVTD HMVSLACSDI LVFLIGMPME FYSIIWNPLT TSSYTLSCKL ETFLFEACSY | 120 |
| ATLLHVLTLS FERYIAICHP FRYKAVSGPC QVKLLIGFVW VTSALVALPL LFAMGTEYPL | 180 |
| VNVPSHRGLT CNRSSTRHHE QPETSNMSIC TNLSSRWTVF QSSIFGAFVV YLVVLLSVAF | 240 |
| MCWNMMQVLM KSQKGSLAGG TRPPQLRKSE SEESRTARRQ TIIFLRLIVV TLAVCWMPNQ | 300 |
| IRRIMAAAKP KHDWTRSYFR AYMILLPFSE TFFYLSSVIN PLLYTVSSQQ FRRVFVQVLC | 360 |
| CRLSLQHANH EKRLRVHAHS TTDSARFVQR PLLFASRRQS SARRTEKIFL STFQSEAEPQ | 420 |
| SKSQSLSLES LEPNSGAKPA NSAAENGFQE HEV | |

Seq ID NO: 152 DNA sequence:
Nucleic Acid Accession #: none found
Coding sequence: 3-65 (underlined sequences correspond to start and stop codons)

| | |
|---|---|
| 1 11 21 31 41 51 | |
| \| \| \| \| \| \| | |
| TTATTATTTT GTGTAAACTA TATTCTGCTT ATAGAGAGTC TCTGAGACTA AAATTGACAA | 60 |
| CTTGAAAAGT ATTCCAAGGA ATATTATCAA AATAGGGCAA CATGGACTGT TTAAGATCTC | 120 |
| CATGTAATTG AAATTCATGC AAGGAAACAA CTCATAGAAA AGATAAATAT GGATGCCCTT | 180 |
| CACATGTTAT CAACCTCGTA ACTTTTGGTG CTTGCTGAAT CAGTCCATGA AAAGCTACAG | 240 |
| CCCGCTCTTT GGGAATGCTA CATACCCATT TCTGGTATTT AAAAAATATC TAGGAGGAGC | 300 |
| TAAATGACAA AACACAGCAG TGTTTTGAGG GAGAAAGGAC CATCATTTAT AATGCTCTGT | 360 |
| ACATACTACC AGAGCTGCTT GGAAAATTAA AGGCCACTTG TGGCTTTTTC CTACCAACTG | 420 |
| ATACGTTTAA ATTTGCCCTA GGATTSAGCT AACAGCAAAA AAAAAAAAAA AAAAAAAARA | 480 |
| GAGAGAAAGA AAGGAGKAAA CAGTGGTAAT AAAAAAATCC ATCTGTCTTC TTGCTATGTT | 540 |
| AATATTAATA AATCATAATA TGACAAGACC CTCACTGAAT AAGAGTATTT TCAGTCATCA | 600 |
| GAAGCCAGCT GTTGGTAGGC ATTAATGAGT TTAAAATTGT TCTCAATTGA AAAAACATCA | 660 |
| CACTATTTTG CCAAAACCAA AGTAATTATA ATACTGTGTC CTCCTGTAAT TTTTTGAGAA | 720 |
| GTGGTTATAA AGGGCATATT TACATAAATT CTACTTTATT CCTCAACTTC TTTGATGAAT | 780 |
| GTAACCCAAT TTTACTTCTT TAAAAAGTCT CAATTCAAGC TCGATTAGCC AGCTCAGCAT | 840 |
| AATCAACTAG ACAGTGGTTT GTTAAATTTA GCAGCATACT TCGTTCCCAT TCTAATTAAA | 900 |
| GTCATGAGTT CTTGAATCCC AGAGAAATAA TGCTTAGGAA CTTCTCTCAA TCTGCTTGGC | 960 |
| TTGGCCTAGA GAAGTGGCCA TTTTATCAAC AGGRAAAAAA AAAATTTTCT CTACTACAAC | 1020 |
| CCCGTTGCCT TCTGAAAAAC AGCAAGTTAT TTCTTTATAT AATTATCATT TTATTATTTT | 1080 |
| ATGGAAAATT AATTTATTAA TTAATAGCCT ATTATGTGTT CTCACTTGCT TCTCTAAGTA | 1140 |
| ATATTTTGAG ATAAAATGTT GAATAAAACC ATGGATTATA GAGAAAAGTC AAAATATATG | 1200 |
| TGTAATATTT AATTATTTTA TAAGTTTTAT AATAAAGTAT TCCATTTCTT TATCTT | |

Seq ID No: 153 Protein sequence:
Protein Accession #: none found

1 11 21 31 41 51
| | | | | |
IILCKLYSAY RESLRLKLTT

Seq ID NO: 154 DNA sequence:
Nucleic Acid Accession #: none found
Coding sequence: 1-36 (underlined sequences correspond to start and stop codons)

| | |
|---|---|
| 1 11 21 31 41 51 | |
| \| \| \| \| \| \| | |
| CTGGATGATA TGGAAGAAAT GGATGGGTTA AGGTAAAAGG CTGATCACAG ATGGGTTCCT | 60 |
| CTCAAGGTTA AAATAGTTTA AGTGCCAGAA GAAAAGGTGG GCACCAGCGA ATTAAGAACC | 120 |
| ATCTTTGAAT GGTCCCCTTG GTTAAATACT TAACTTTTGT CATCAGTGTC TGCATTTATG | 180 |
| AAATGAAGAG GAATTCACTA ATATGCTACG TGATCTTTTG TTTGTCATGA AAAGAGTTAC | 240 |
| TGTTGTGTAG TTCTCTGTTC CAGGGCTGCC TTTGCTCCAC AAAGCACTGA GAAGCAGTGG | 300 |
| CCCTGTACAA CCATACTGCC TCTCAACACT GTGTAATAGG CTAACACCGC CCAGCGAACC | 360 |
| TTCCTGGGAG ATATAAAATA CATAGGTTTA GGCTGGCAAA AAAAAAAAAA AAA | |

Seq ID NO: 155 Protein sequence:
Piotein Accession #: none found

1 11 21 31 41 51
| | | | | |
LDDMEEMDGL R

Seq ID NO: 156 DNA sequence:
Nucleic Acid Accession #: NM_032961.1
Coding sequence: 827-3949 (underlined sequences correspond to start and stop codons)

| | |
|---|---|
| 1 11 21 31 41 51 | |
| \| \| \| \| \| \| | |
| CAGGCTCAGA GGCTGAAGCA GGAGGAAGGA AGGACTGGAA GGAAAAAGAG ACAGGTTAGA | 60 |
| GGGAAAGAGG CTTGGGAAGA AAACAGCAGA AAAGAAACTG CTCATTACAC TTACAGAGAG | 120 |
| GCAAGTAACG GTGGAGATGA GGACAGAGGG AACCAAGACT CTGAAAGACA AAAAATACAA | 180 |
| ATAGAGCGAA AGAGGAAAAA AATGTCAAGA AGAACATCCA TCCGGAGAAA TGAAGAGAAT | 240 |
| GAAAGTTTTA AACTGCAGAG CCGTTCTGTG CTTTTCCGGC ACAAAATTAT ATCGCTGATT | 300 |
| TTAAGCCCTT TTGCATTTGC CAGCCGTTGA CATTAAGAGG CATGTTTAAC GGTGCCAACA | 360 |
| GCATCTCCTT TTCCTTCTCC TCTTCCTCTT CTTCTTCTTC CTCCTCCTCC TCCTCTTTTT | 420 |
| CCTCCTCCTC GTTCTCCTCC CATCAGCAAG AAGACAAACC GAGGACAGTC TTGAAATATC | 480 |
| GAAATTTCCT CTTTGGGATT TGCCAGCGCC AAGACTGTCG GAATAAAGGA CGCTGACTAT | 540 |
| TGTATTATTG TTATTTTATT AATTAGTCAG TGGAAAGATT ACAGATGAGG AAAGGGGACG | 600 |
| CCTGTCACCC TTCCTGTGCT AAGATTTAAA AAAAAATGAG GCTGGATTGC GGGAAGCTCT | 660 |
| AAAATGAAGC AAAAGGAGTA AGATTTTTAA AGACAGAAAG CCACAGGAGC CCCCACGTAG | 720 |
| CGCACTTTTA TTTGTATTTT TTCAGATTTT TTTTTGTTTC GTGGTGGTGG GGGAGGTGAT | 780 |
| TGGGTGGCTG ACTGGCTGCG GGAAGCTACT TCCTTTCCTT TTGGAGATGA TTGTGCTATT | 840 |
| ATTGTTTGCC TTGCTCTGGA TGGTGGAAGG AGTCTTTTCC CAGCTTCACT ACACGGTACA | 900 |
| GGAGGAGCAG GAACATGGCA CTTTCGTGGG GAATATCGCT GAAGATCTGG GTCTGGACAT | 960 |
| TACAAAACTT TCGGCTCGCG GGTTTCAGAC GGTGCCCAAC TCAAGGACCC CTTACTTAGA | 1020 |
| CCTCAACCTG GAGACAGGGG TGCTGTACGT GAACGAGAAA ATAGACCGCG AACAAATCTG | 1080 |
| CAAACAGAGC CCCTCCTGTG TCCTGCACCT GGAGGTCTTT CTGGAGAACC CCCTGGAGCT | 1140 |
| GTTCCAGGTG GAGATCGAGG TGCTGGACAT TAATGACAAC CCCCCCTCTT TCCCGGAGCC | 1200 |
| AGACCTGACG GTGGAAATCT CTGAGAGCGC CACGCCAGGC ACTCGCTTCC CCTTGGAGAG | 1260 |
| CGCATTCGAC CCAGACGTGG GCACCAACTC CTTGCGCGAC TACGAGATCA CCCCCAACAG | 1320 |
| CTACTTCTCC CTGGACGTGC AGACCCAGGG GGATGGCAAC CGATTCGCTG AGCTGGTGCT | 1380 |
| GGAGAAGCCA CTGGACCGAG AGCAGCAAGC GGTGCACCGC TACGTGCTGA CCGCGGTGGA | 1440 |
| CGGAGGAGGT GGGGGAGGAG TAGGAGAAGG AGGGGGAGGT GGCGGGGGAG CAGGCCTGCC | 1500 |
| CCCCCAGCAG CAGCGCACCG GCACGGCCCT ACTCACCATC CGAGTGCTGG ACTCCAATGA | 1560 |
| CAATGTGCCC GCTTTCGACC AACCCGTCTA CACTGTGTCC CTACCAGAGA ACTCTCCCCC | 1620 |
| AGGCACTCTC GTGATCCAGC TCAACGCCAC CGACCCGGAC GAGGGCCAGA ACGGTGAGGT | 1680 |
| CGTGTACTCC TTCAGCAGCC ACATTTCGCC CCGGGCGCGG GAGCTTTTCG GACTCTCGCC | 1740 |
| GCGCACTGGC AGACTGGAGG TAAGCGGCGA GTTGGACTAT GAAGAGAGCC CAGTGTACCA | 1800 |
| AGTGTACGTG CAAGCCAAGG ACCTGGGCCC CAACGCCGTG CCTGCGCACT GCAAGGTGCT | 1860 |
| AGTGCGAGTA CTGGATGCTA ATGACAACGC GCCAGAGATC AGCTTCAGCA CCGTGAAGGA | 1920 |
| AGCGGTGAGT GAGGCGGCGG CGCCCGGCAC TGTGGTGGCC CTTTTCAGCG TGACTGACCG | 1980 |
| CGACTCAGAG GAGAATGGGC ACGTGCAGTG CGAGCTACTG GGAGACGTGC CTTTCCGCCT | 2040 |
| CAAGTCTTCC TTTAAGAATT ACTACACCAT CGTTACCGAA GCCCCCCTGG ACCGAGAGGC | 2100 |
| GGGGGACTCC TACACCCTGA CTGTAGTGGC TCGGGACCGG GGCGAGCCTG CGCTCTCCAC | 2160 |
| CAGTAAGTCG ATCCAGGTAC AAGTGTCGGA TGTGAACGAC AACGCGCCGC GTTTCAGCCA | 2220 |
| GCCGGTCTAC GACGTGTATG TGACTGAAAA CAACGTGCCT GGCGCCTACA TCTACGCGGT | 2280 |
| GAGCGCCACC GACCGGGATG AGGGCGCCAA CGCCCAGCTT GCCTACTCTA TCCTCGAGTG | 2340 |
| CCAGATCCAG GGCATGAGCG TCTTCACCTA CGTTTCTATC AACTCTGAGA ACGGCTACTT | 2400 |
| GTACGCCCTG CGCTCCTTCG ACTATGAGCA GCTGAAGGAC TTCAGTTTTC AGGTGGAAGC | 2460 |
| CCGGGACGCT GGCAGCCCCC AGGCGCTGGC TGGTAACGCC ACTGTCAACA TCCTCATAGT | 2520 |
| GGATCAAAAT GACAACGCCC CTGCCATCGT GGCGCCTCTA CCAGGGCGCA ACGGGACTCC | 2580 |
| AGCGCGTGAG GTGCTGCCCC GCTCGGCGGA GCCGGGTTAC CTGCTCACCC GCGTGGCCGC | 2640 |
| CGTGGACGCG GACGACGGCG AGAACGCCCG GCTCACTTAC AGCATCGTGC GTGGCAACGA | 2700 |
| AATGAACCTC TTTCGCATGG ACTGGCGCAC CGGGGAGCTG CGCACAGCAC GCCGAGTCCC | 2760 |
| GGCCAAGCGC GACCCCCAGC GGCCTTATGA GCTGGTGATC GAGGTGCGCG ACCATGGGCA | 2820 |
| GCCGCCCCTT TCCTCCACCG CCACCCTGGT GGTTCAGCTG GTGGATGGCG CCGTGGAGCC | 2880 |
| CCAGGGCGGG GGCGGGAGCG GAGGCGGAGG GTCAGGAGAG CACCAGCGCC CCAGTCGCTC | 2940 |
| TGGCGGCGGG GAAACCTCGC TAGACCTCAC CCTCATCCTC ATCATCGCGT TGGGCTCGGT | 3000 |
| GTCCTTCATC TTCCTGCTGG CCATGATCGT GCTGGCCGTG CGTTGCCAAA AAGAGAAGAA | 3060 |
| GCTCAACATC TATACTTGTC TGGCCAGCGA TTGCTGCCTC TGCTGCTGCT GCTGCGGTGG | 3120 |
| CGGAGGTTCG ACCTGCTGTG GCCGCCAAGC CCGGGCGCGC AAGAAGAAAC TCAGCAAGTC | 3180 |
| AGACATCATG CTGGTGCAGA GCTCCAATGT ACCCAGTAAC CCGGCCCAGG TGCCGATAGA | 3240 |
| GGAGTCCGGG GGCTTTGGCT CCCACCACCA CAACCAGAAT TACTGCTATC AGGTATGCCT | 3300 |
| GACCCCTGAG TCCGCCAAGA CCGACCTGAT GTTTCTTAAG CCCTGCAGCC CTTCGCGGAG | 3360 |
| TACGGACACT GAGCACAACC CCTGCGGGGC CATCGTCACC GGTTACACCG ACCAGCAGCC | 3420 |
| TGATATCATC TCCAACGGAA GCATTTTGTC CAACGAGACT AAACACCAGC GAGCAGAGCT | 3480 |
| CAGCTATCTA GTTGACAGAC CTCGCCGAGT TAACAGTTCT GCATTCCAGG AAGCCGACAT | 3540 |
| AGTAAGCTCT AAGGACAGTG GTCATGGAGA CAGTGAACAG GGAGATAGTG ATCATGATGC | 3600 |
| CACCAACCGT GCCCAGTCAG CTGGTATGGA TCTCTTCTCC AATTGCACTG AGGAATGTAA | 3660 |
| AGCTCTGGGC CACTCAGATC GGTGCTGGAT GCCTTCTTTT GTCCCTTCTG ATGGACGCCA | 3720 |
| GGCTGCTGAT TATCGCAGCA ATCTGCATGT TCCTGGCATG GACTCTGTTC CAGACACTCA | 3780 |
| GGTGTTTGAA ACTCCAGAAG CCCAGCCTGG GGCAGAGCGG TCCTTTTCCA CCTTTGGCAA | 3840 |
| AGAGAAGGCC CTTCACAGCA CTCTGGAGAG GAAGGAGCTG GATGGACTGC TGACTAATAC | 3900 |
| GCGAGCGCCT TACAAACCAC CATATTTGAC ACGGAAAAGG ATATGCTAGT CAATTCTACA | 3960 |
| GGACTTACCT GAAGCAGCAT GATTTGCACA AAGTCGACCA ACAAAAGCAT CAACTTTTCA | 4020 |
| ACTTCATTAT CTTGGCCATC CAGTTAGTCA TGTGTAACTG AGTATTAGAT TTCGGATGGA | 4080 |
| GTCATCATGG CCAATTATAG GACCTAATTG CTCTCAGCAG GCCTGAGAAA TGAGTTGAAA | 4140 |
| TGTGCAGAAC TGTAGAAACT TTAGAGGCAA CAGATTTTGC CTCCCCGATC AGTGTGTGCC | 4200 |
| TGTTTACAGC ACTATCTATC TTTCTCTCTC CAAATGTCAC TCAGCCCTTT AGATGTTTAT | 4260 |
| ATTCACCACG AGAAGCCAGT CATAAAGATA AAGGAAATTT GTGCATTATA AATGCAATAT | 4320 |
| CACTGTTTTA AACTTGACTG TTTTATATTA TTTTTGTGTG ATCAAGTGTT CCGCAAGCTA | 4380 |
| TTCCAACTTT ACAAGAGAAA TTGTGATTAT GTTCTTTTCA CCTGTGGGTT ATAAAAAATG | 4440 |
| TTGTATTCTG AAGACCCACA AAATATCAAA GACATTCTGT AGTTTATACA CCGTGTTGCA | 4500 |
| AAGTGTTTAC TGTACTATTT CAAAGCTTCT AAATAAATAT AAAATATATA TATTATATTA | 4560 |
| TATAATTTTC CTAAAATGTG GTACAACTCA GTTGGTTTTT AAATGGATGC ATACAGTCCA | 4620 |
| CATCATACAA TAAAATAAAA GGTAATTCAG GGTCCCAAAG ACAAACTTAC TAAGAAAAAA | 4680 |
| TCATTAATAG TTTTCTCCCA ATTTCCATAT CTTACTCAAC CGTGTTTTTC CTTGTTTAAA | 4740 |
| AGAAAATGAT GCTCTAAGCT ACAAAATTTT GTCAAAAACT CATATTGAAT TTTCAATGCC | 4800 |
| AAAGATGTAG CTATTCATGT TATCAGACAG AGCACTGACT ATGTACTATC AAACTATCTA | 4860 |
| ACAATCTGCA TAAGTCTGAT TCTATTTCTA TGACTTTGAA TTTAGAATCA CTTAAAGCTT | 4920 |
| TTATAAAGAA TCGATAAATT CACCTGTATT TGTTGTTAGA AAAAAACTGG GTGTCTGTAC | 4980 |
| ATTTTGTGGT GTAAAATATG TAATTGAAGA TTACTATTTT AAGAAGTCAT CAGTCATATC | 5040 |
| ACTCACACAG AATTTTATTT TACATAGTTT TGTGACTTAA TTACACATGA ATATAAAATC | 5100 |
| TATAATTCTA TATGAATATA TAGAGATATA GAAACATCTG AACTGGTAAA GAATAACTAT | 5160 |
| AAAATATGAA AGCTCTAAAT TTAAAATAAA TTTAGAGATA GAATCATGGT ACATTATTGT | 5220 |
| TTCAGTATTC CATGTAAAAA TTTTATAGCT TAAATGTAGT CAGTGTTTGA TTAATGAAAA | 5280 |
| AATTCTTCAT GAGTCAGCCT TCAAAAGTTA AGCTTGCCTT TTACTTTTAT GTCAACAATA | 5340 |
| TTAATTATTA AATTTAGTAA GACGCAAAAA AAAAAAAAAA AAAA | |

Seq ID No: 157 Protein sequence:
Protein Accession #: NP_116586.1

| | |
|---|---|
| 1 11 21 31 41 51 | |
| \| \| \| \| \| \| | |
| MIVLLLFALL WMVEGVFSQL HYTVQEEQEH GTFVGNTAED LGLDITKLSA RGFQTVPNSR | 60 |
| TPYLDLNLET GVLYVNEKID REQICKQSPS CVLHLEVFLE NPLELFQVEI EVLDINDNPP | 120 |
| SFPEPDLTVE ISESATPGTR FPLESAFDPD VGTNSLRDYE ITPNSYFSLD VQTQGDGNRF | 180 |
| AELVLEKPLD REQQAVHRYV LTAVDGGGGG GVGEGGGGGG GAGLPPQQQR TGTALLTIRV | 240 |
| LDSNDNVPAF DQPVYTVSLP ENSPPGTLVI QLNATDPDEG QNGEVVYSFS SHISPRAREL | 300 |
| FGLSPRTGRL EVSGELDYEE SPVYQVYVQA KDLGPNAVPA HCKVLVRVLD ANDNAPEISF | 360 |
| STVKEAVSEG AAPGTVVALF SVTDRDSEEN GQVQCELLGD VPFRLKSSFK NYYTTVTEAP | 420 |
| LDREAGDSYT LTVVARDRGE PALSTSKSIQ VQVSDVNDNA PRFSQPVYDV YVTENNVPGA | 480 |
| YIYAVSATDR DEGANAQLAY SILECQIQGM SVFTYVSINS ENGYLYALRS FDYEQLKDFS | 540 |
| FQVEARDAGS PQALAGNATV NILIVDQNDN APAIVAPLPG RNGTPAREVL PRSAEPGYLL | 600 |
| TRVAAVDADD GENARLTYSI VRGNEMNLFR MDWRTGELRT ARRVPAKRDP QRPYELVIEV | 660 |
| RDHGQPPLSS TATLVVQLVD GAVEPQGGGG SGGGGSGEHQ RPSRSGGGET SLDLTLILII | 720 |
| ALGSVSFIFL LAMIVLAVRC QKEKKLNIYT CLASDCCLCC CCCGGGGSTC CGRQARARKK | 780 |
| KLSKSDIMLV QSSNVPSNPA QVPIEESGGF GSHHHNQNYC YQVCLTPESA KTDLMFLKPC | 840 |
| SPSRSTDTEH NPCGAIVTGY TDQQPDIISN GSILSNETKH QRAELSYLVD RPRRVNSSAF | 900 |
| QEADIVSSKD SGHGDSEQGD SDHDATNRAQ SACMDLFSNC TEECKALGNS DRCWMPSFVP | 960 |
| SDCRQAADYR SNLHVPGMDS VPDTEVFETP EAQPGAERSF STFGKEKALH STLERKELDG | 1020 |
| LLTNTRAPYK PPYLTRKRIC | |

Seq ID NO: 158 DNA sequence:
Nucleic Acid Accession #: NM_022159.1
Coding sequence: 70-1890 (underlined sequences correspond to start and stop codons)

| | |
|---|---|
| 1 11 21 31 41 51 | |
| \| \| \| \| \| \| | |
| GTGAAATTTA AACTCCAGTC CTGTGGCGAA AATGCTAATT GCACTAACAC AGAAGGAAGT | 60 |
| TATTATTGTA TGTGTGTACC TGGCTTCAGA TCCAGCAGTA ACCAAGACAG GTTTATCACT | 120 |
| AATGATGGAA CCGTCTGTAT AGAAAATGTG AATGCAAACT GCCATTTAGA TAATGTCTGT | 180 |
| ATAGCTGCAA ATATTAATAA AACTTTAACA AAAATCAGAT CCATAAAAGA ACCTGTGGCT | 240 |
| TTGCTACAAG AAGTCTATAG AAATTCTGTG ACAGATCTTT CACCAACAGA TATAATTACA | 300 |
| TATATAGAAA TATTAGCTGA ATCATCTTCA TTACTAGGTT ACAAGAACAA CACTATCTCA | 360 |
| GCCAAGGACA CCCTTTCTAA CTCAACTCTT ACTGAATTTG TAAAAACCGT GAATAATTTT | 420 |
| GTTCAAAGGG ATACATTTGT AGTTTGGGAC AAGTTATCTG TGAATCATAG GAGAACACAT | 480 |
| CTTACAAAAC TCATGCACAC TGTTGAACAA GCTACTTTAA GGATATCCCA GAGCTTCCAA | 540 |
| AAGACCACAG AGTTTGATAC AAATTCAACG GATATAGCTC TCAAAGTTTT CTTTTTTGAT | 600 |
| TCATATAACA TGAAACATAT TCATCCTCAT ATGAATATGG ATGGAGACTA CATAAATATA | 660 |
| TTTCCAAAGA GAAAAGCTGC ATATGATTCA AATGGCAATG TTGCAGTTGC ATTTTTATAT | 720 |
| TATAAGAGTA TTGGTCCTTT GCTTTCATCA TCTGACAACT TCTTATTGAA ACCTCAAAAT | 780 |
| TATGATAATT CTGAAGAGGA GGAAAGAGTC ATATCTTCAG TAATTTCAGT CTCAATGAGC | 840 |
| TCAAACCCAC CCACATTATA TGAACTTGAA AAAATAACAT TTACATTAAG TCATCGAAAG | 900 |
| GTCACAGATA GGTATAGGAG TCTATGTGCA TTTTGGAATT ACTCACCTGA TACCATGAAT | 960 |
| GGCAGCTGGT CTTCAGAGGG CTGTGAGCTG ACATACTCAA ATGAGACCCA CACCTCATGC | 1020 |
| CGCTGTAATC ACCTGACACA TTTTGCAATT TTGATGTCCT CTGGTCCTTC CATTGGTATT | 1080 |
| AAAGATTATA ATATTCTTAC AAGGATCACT CAACTAGGAA TAATTATTTC ACTGATTTGT | 1140 |
| CTTGCCATAT GCATTTTTAC CTTCTGGTTC TTCAGTGAAA TTCAAAGCAC CAGGACAACA | 1200 |
| ATTCACAAAA ATCTTTGCTG TAGCCTATTT CTTGCTGAAC TTGTTTTTCT TGTTGGGATC | 1260 |
| AATACAAATA CTAATAAGCT CTTCTGTTCA ATCATTGCCG GACTGCTACA CTACTTCTTT | 1320 |
| TTAGCTGCTT TTGCATGGAT GTGCATTGAA GGCATACATC TCTATCTCAT TGTTGTGGGT | 1380 |
| GTCATCTACA ACAAGGGATT TTTGCACAAG AATTTTTATA TCTTTGGCTA TCTAAGCCCA | 1440 |
| GCCGTGGTAG TTGGATTTTC GGCAGCACTA GGATACAGAT ATTATGGCAC AACCAAAGTA | 1500 |
| TGTTGGCTTA GCACCGAAAA CAACTTTATT TGGAGTTTTA TAGGACCAGC ATGCCTAATC | 1560 |
| ATTCTTGTTA ATCTCTTGGC TTTTGGAGTC ATCATATACA AAGTTTTTCG TCACACTGCA | 1620 |
| GGGTTGAAAC CAGAAGTTAG TTGCTTTGAG AACATAAGGT CTTGTGCAAG AGGAGCCCTC | 1680 |
| GCTCTTCTGT TCCTTCTCGG CACCACCTGG ATCTTTGGGG TTCTCCATGT TGTGCACGCA | 1740 |
| TCAGTGGTTA CAGCTTACCT CTTCACAGTC AGCAATGCTT TCCAGGGGAT GTTCATTTTT | 1800 |
| TTATTCCTGT GTGTTTTATC TAGAAAGATT CAAGAAGAAT ATTACAGATT GTTCAAAAAT | 1860 |
| GTCCCCTGTT GTTTTGGATG TTTAAGGTAA ACATAGAGAA TGGTGGATAA TTACAACTGC | 1920 |
| ACAAAAATAA AAATTCCAAG CTGTGGATGA CCAATGTATA AAAATGACTC ATCAAATTAT | 1980 |
| CCAATTATTA ACTACTAGAC AAAAAGTATT TTAAATCAGT TTTTCTGTTT ATGCTATAGG | 2040 |
| AACTGTAGAT AATAAGGTAA AATTATGTAT CATATAGATA TACTATGTTT TTCTATGTGA | 2100 |
| AATAGTTCTG TCAAAAATAG TATTGCAGAT ATTTGGAAAG TAATTGGTTT CTCAGGAGTG | 2160 |
| ATATCACTGC ACCCAAGGAA AGATTTTCTT TCTAACACGA GAAGTATATG AATGTCCTGA | 2220 |
| AGGAAACCAC TGGCTTGATA TTTCTGTGAC TCGTGTTGCC TTTGAAACTA GTCCCCTACC | 2280 |
| ACCTCGGTAA TGAGCTCCAT TACAGAAAGT GGAACATAAG AGAATGAAGG GGCAGAATAT | 2340 |
| CAAACAGTGA AAAGGGAATG ATAAGATGTA TTTTGAATGA ACTGTTTTTT CTGTAGACTA | 2400 |
| GCTGAGAAAT TGTTGACATA AAATAAAGAA TTGAAGAAAC ACATTTTACC ATTTTGTGAA | 2460 |
| TTGTTCTGAA CTTAAATGTC CACTAAAACA ACTTAGACTT CTGTTTGCTA AATCTGTTTC | 2520 |
| TTTTTCTAAT ATTCTAAAA | |

Seq ID No: 159 Protein sequence:
Protein Accession #: NP_071442.1

| | |
|---|---|
| 1 11 21 31 41 51 | |
| \| \| \| \| \| \| | |
| MCVPGFRSSS NQDRFITNDG TVCIENVNAN CHLDNVCIAA NINKTLTKIR SIKEPVALLQ | 60 |
| EVYRNSVTDL SPTDIITYIE ILAESSSLLG YKNNTISAKD TLSNSTLTEF VKTVNNFVQR | 120 |
| DTFVVWDKLS VNHRRTHLTK LMHTVEQATL RISQSFQKTT EFDTNSTDIA LKVFFFDSYN | 180 |
| MKHIHPHMNM DGDYINIFPK RKAAYDSNGN VAVAFLYYKS IGPLLSSSDN FLLKPQNYDN | 240 |
| SEEEERVISS VISVSMSSNP PTLYELEKIT FTLSHRKVTD RYRSLCAFWN YSPDTMNGSW | 300 |
| SSEGCELTYS NETHTSCRCN HLTHFAILMS SGPSIGIKGY NILTRITQLG IIISLICLAI | 360 |
| CIFTFWFFSE IQSTRTTIHK NLCCSLFLAE LVFLVGINTN TNKLFCSIIA GLLHYFFLAA | 420 |
| FAWMCIEGIH LYLIVVGVIY NKGFLHKNFY IFGYLSPAVV VGFSAALGYR YYGTTKVCWL | 480 |
| STENNFIWSF IGPACLIILV NLLAFGVIIY KVFRHTAGLK PEVSCFENIR SCARGALALL | 540 |
| FLLGTTWIFG VLHVVHASVV TAYLETYSNA FQGMFIFLFL CVLSRKIQEE YYRLFKNVPC | 600 |
| CFGCLR | |

Seq ID NO: 160 DNA sequence:
Nucleic Acid Accession #: none found
Coding sequence: 1-216 (underlined sequences correspond to start and stop codons)

| | |
|---|---|
| 1 11 21 31 41 51 | |
| \| \| \| \| \| \| | |
| TGTCTGCTTA TGCGGTGGCT CGCTGCTCAG AACAGGATGG CAGAGATGAG CACCACCATC | 60 |
| AAAAACTCAA GGACCAGTGC TGTGGGTCCA GTCATCTGTT TCATGGAATT CACCAGTCTG | 120 |
| GTATCTTCAA AATCCAGAAG GATGATGGCA GATGGCAGGA AGGAGGAAGA GGGTAATCTG | 180 |
| GAAGAGTTTC CTGACCTACT CTGCTGCTGT GATTAAACAA CCACCAGGAAATTTTGATGA | 240 |
| CACTGTTCTC CTGAGCTCCT CCCTTTCCTC GGGGAAGAAA AGCATTGAAA CTACAAAAAT | 300 |
| AAAGTGTTAT TTGGCTGGAG TGAGGTCTCA TGTCTGCTTA TGCGGTGGCT CGCTGCTCAG | 360 |
| AACAGGGAAC CATTGGAGAT ACTCATTACT CTTTGAAGGC TTACAGTGGA ATGAATTCAA | 420 |
| ATACGACTTA TTTGAGGAAT TGAAGTTGAC TTTATGGAGC TGATAAGAAT CTTCTTGGAG | 480 |
| AAAAAAAGAC TGGTACTTCT GAATTAACCA AAATCACAGT ATTCTGAAGA TGATTCTACA | 540 |
| AAGCCTGCTG TTTCTACAAA GGCTGCTGAT GATTTCTACA AAGCCTGCTG TAGTGTTGCT | 600 |
| GTGGCCTCTG CTTAAAAAAG TAGAAAACAC ATTGATGCAG CATGTTCACC CCAACCTCCC | 660 |
| TGCCTAAAGG CTCAGGGACC ATCTTGGAAG AGGAAGGCGC GTGAGATTGT AAGAGCCGAA | 720 |
| TTAGGGGGAT GGAGTGTGGA GAATAAGGAC ACTTCATCTT GGATGCTCAC CTGCCAAATT | 780 |
| GACTTCTGAT GAAAGCCAGC TCCAGAAATG TGCCTACAGT TACTACTTTC ACCTAAACCC | 840 |
| TGCCCTTAGT CAAATCCTTC TCTTCTTCTA AGCAATCAAC TTCAATTCCT TGTATAACCC | 900 |
| ACAGTATAAA AGGGCTTTTA TACCATTCTA TCCTATTGCA TGTAAGCCTT GGGTCTGGGA | 960 |
| GGTAACAGTG TGGGATTCCA CCATCTCATC TCCCTGCCAC CCAAACATGC CTGCTCTTCT | 1020 |
| TTAAGCAATA TTAAATGTTT GTACTTCA | |

Seq ID No: 161 Protein sequence:
Protein Accession #: none found

| | |
|---|---|
| 1 11 21 31 41 51 | |
| \| \| \| \| \| \| | |
| CLLMRWLAAQ NRMAEMSTTI KNSRTSAVGP VICFMEFTSL VSSKSRRMMA DGRKEEEGNL | 60 |
| EEFPDLLCCC D | |

Seq ID NO: 162 DNA sequence:
Nucleic Acid Accession #: none found
Coding sequence: 1-159 (underlined sequences correspond to start end stop codons)

| | |
|---|---|
| 1 11 21 31 41 51 | |
| \| \| \| \| \| \| | |
| GAGACCCTCC AGAGGCAGGG CCCAGGATTG AAGAGGGAAG CCCTGCTCCA CACGTGTTCA | 60 |
| TCAGGAAGGA CCCACAGACT GCTGCTCCTG GAGGCCTCTC GGTTTATGGA TGTGTGTTTG | 120 |
| TTCCATAAAC CCTCAGAGGG TCACCTGGAG ACCCGCTAAA ATGCAGGTTC TTGGGCCACA | 180 |
| TCCTAGACCT TCTGACCGAC CCAGGGAGTG GGGCCCAGGA AGCTGCATTT GACAGATATC | 240 |
| CCCGTGTGAT CATCATGCAC ACAGGAGTGA GAGAACCAGT GTTCTCCCCG GGCAGAAGGG | 300 |
| AAGCTCGTGT GCAGGACACC TCACACCTCC TTTCCCATTC CCCTGCCAGG CTCTCCCTGC | 360 |
| TGACATTGTT TTTGCGGGAG AGCTGTGAAT TCTGAAGATT AGGTTGCTTC TCACCCCAAG | 420 |
| CTCCAGAAGT CCAGGCTGAG CCAAACCAAG CTTCAAGTTG TGCCTGGACT TGGAGAACCA | 480 |
| GGAGGTGAGG GGACTGACTA CTTGAAGATC ACATGGAGGA GGAGTCTGAT CCAGGCCCAG | 540 |
| GCACCAAGGA AAGGCCATGC AAGGACACAG GGAGAAGGGC AGCTGTCTGT AAGCCAGAAA | 600 |
| GAGCCTTCAC TAGAAACCAA ATCAGCCAGA ACCTTCATCT TGGACTTTCC AGCCTTCAGA | 660 |
| GATGTGAAAA AATAAATTTC TGTTGATTAA CCTAAAAAA | |

Seq ID No: 163 Protein sequence:
Protein Accession #: none found

1 11 21 31 41 51
| | | | | |
ETLQRQGPGL KREALLHTCS SGRTHRLLLL EASRFMDVCL FHKPSEGHLE TR

Seq ID NO: 164 DNA sequence:
Nucleic Acid Accession #: NM_020241.1
Coding sequence: 4-1557 (underlined sequences correspond to start and stop codons)

| | |
|---|---|
| 1 11 21 31 41 51 | |
| \| \| \| \| \| \| | |
| GCCATGCAGA CCCCGCGAGC GTCCCCTCCC CGCCCGGCCC TCCTGCTTCT GCTGCTGcTA | 60 |
| CTGGGGGGCG CCCACGGCCT CTTTCCTGAG GAGCCGCCGC CGCTTAGCGT GGCCCCCAGG | 120 |
| GACTACCTGA ACCACTATCC CGTGTTTGTG GGCAGCGGGC CCGGACGCCT GACCCCCGCA | 180 |
| GAAGGTGCTG ACGACCTCAA CATCCAGCGA GTCCTGCGGG TCAACAGGAC GCTGTTCATT | 240 |
| GGGGACAGGG ACAACCTCTA CCGCGTAGAG TTGGAGCCCC CCACGTCCAC GGAGCTGCGG | 300 |
| TACCAGAGGA AGCTGACCTG GAGATCTAAC CCCAGCGACA TAAACGTGTG TCGGATGAAG | 360 |
| GGCAAACAGG AGGGCGAGTG TCGAAACTTC GTAAAGGTGC TGCTCCTTCG GGACGAGTCC | 420 |
| ACGCTCTTTG TGTGCGGTTC CAACGCCTTC AACCCGGTGT GCGCCAACTA CAGCATAGAC | 480 |
| ACCCTGCAGC CCGTCGGAGA CAACATCAGC GGTATGGCCC GCTGCCCGTA CGACCCCAAG | 540 |
| CACGCCAATG TTGCCCTCTT CTCTGACGGG ATGCTCTTCA CAGCTACTGT TACCGACTTC | 600 |
| CTAGCCATTG ATGCTGTCAT CTACCGCAGC CTCGGGGACA GGCCCACCCT GCGCACCGTG | 660 |
| AAACATGACT CCAAGTGGTT CAAAGAGCCT TACTTTGTCC ATGGGGTGGA GTGGGGCAGC | 720 |
| CATGTCTACT TCTTCTTCCG GGAGATTGCG ATGGAGTTTA ACTACCTGGA GAAGGTGGTG | 780 |
| GTGTCCCGCG TGGCCCGAGT GTGCAAGAAC GACGTGGGAG GCTCCCCCCG CGTGCTGGAG | 840 |
| AAGCAGTGGA CGTCCTTCCT GAAGGCGCGG CTCAACTGCT CTGTACCCGG AGACTCCCAT | 900 |
| TTCTACTTCA ACGTGCTGCA GGCTGTCACG GGCGTGGTCA GCCTCGGGGG CCGGCCCGTG | 960 |
| GTCCTGGCCG TTTTTTCCAC GCCCAGCAAC AGCATCCCTG GCTCGGCTGT CTGCGCCTTT | 1020 |
| GACCTGACAC AGGTGGCAGC TGTGTTTGAA GGCCGCTTCC GAGAGCAGAA GTCCCCCGAG | 1080 |
| TCCATCTGGA CGCCGGTGCC GGAGGATCAG GTGCCTCGAC CCCGGCCCGG GTGCTGCGCA | 1140 |
| GCCCCCGGGA TGCAGTACAA TGCCTCCAGC GCCTTGCCGG ATGACATCCT CAACTTTGTC | 1200 |
| AAGACCCACC CTCTGATGGA CGAAGCGGTG CCCTCGCTGG GCCATGCGCC CTGGATCCTG | 1260 |
| CGGACCCTGA TGAGGCACCA GCTGACTCGA GTGGCTGTGG ACGTGGGAGC CGGCCCCTGG | 1320 |
| GGCAACCAGA CCGTTGTCTT CCTGGGTTCT GAGGCGGGGA CGGTCCTCAA GTTCCTCGTC | 1380 |
| CGGCCCAATG CCAGCACCTC AGGGACGTCT GGGCGTGTGT GTCAAGTGGG CCACGCGTGC | 1440 |
| AGGGTGTGTG TCCACGAGCG ACGATCGTGG TGGCCCCAGC GGCCTGGGCG TTGGCTGAGC | 1500 |
| CGACGCTGGG GCTTCCAGAA GGCCCGGGGG CCTCCGAGGT GCCGGTTAGG AGTTTGAACC | 1560 |
| CCCCCCACTC TGCAGAGGGA AGCGGGGACA ATGCCGGGGT TTCAGGCAGG AGACACGAGG | 1620 |
| AGGGCCTGCC CGGAAGTCAC ATCGGCAGCA GCTGTCTAAA GGGCTTGGGG GCCTGGGGGG | 1680 |
| CGGCGAAGGT GGGTGGGGCC CCTCTGTAAA TACGGCCCCA GGGTGGTGAG AGAGTCCCAT | 1740 |
| GCCACCCGTC CCCTTGTGAC CTCCCCCCTC TGACCTCCAG CTGACCATGC ATGCCACGTG | 1800 |
| G | |

Seq ID No: 165 Protein sequence:
Protein Accession #: NP_064626.1

| | |
|---|---|
| 1 11 21 31 41 51 | |
| \| \| \| \| \| \| | |
| MQTPRASPPR PALLLLLLLL GGAHGLFPEE PPPLSVAPRD YLNHYPVFVG SGPGRLTPAE | 60 |
| GADDLNIQRV LRVNRTLFIG DRDNLYRVEL EPPTSTELRY QRKLTWRSNP SDINVCRMKG | 120 |
| KQEGECRNFV KVLLLRDEST LFVCGSNAFN PVCANYSIDT LQPVGDNISG MARCPYDPKH | 180 |
| ANVALFSDGM LFTATVTDFL AIDAVIYRSL GDRPTLRTVK HDSKWFKEPY FVHAVEWGSH | 240 |
| VYFFFREIAM EFNYLEKVVV SRVARVCKND VGGSPRVLEK QWTSFLKARL NCSVPGDSHF | 300 |
| YFNVLQAVTG VVSLGGRPVV LAVFSTPSNS IPGSAVCAFD LTQVAAVFEG RFREQKSPES | 360 |
| IWTPVPEDQV PRPRPGCCAA PGMQYNASSA LPDDILNFVK THPLMDEAVP SLGHAPWILR | 420 |
| TLMRHQLTRV AVDVGAGPWG NQTVVFLGSE AGTVLKFLVR PNASTSGTSG RVCQVGHACR | 480 |
| VCVHERRSWW PQRPGRWLSR RGFQKARGPP RCRLGV | |

Seq ID NO: 166 DNA sequence:
Nucleic Acid Accession #: NM_032108.1
Coding sequence: 39-2705 (underlined sequences correspond to start and stop codons)

| | |
|---|---|
| 1 11 21 31 41 51 | |
| \| \| \| \| \| \| | |
| TCCGAGGCGT CACCTCCTCC TGTCGCCTGG CCCTCGCCAT GCAGACCCCG CGAGCGTCCC | 60 |
| CTCCCCGCCC GGCCCTGCTG CTTCTGCTGC TGCTACTGGG GGGCGCCCAC GGCCTCTTTC | 120 |
| CTGAGGACCC GCCGCCGCTT AGCGTGGCCC CCAGGGACTA CCTGAACCAC TATCCCGTGT | 180 |
| TTGTGGGCAG CGGGCCCGGA CGCCTGACCC CCGCAGAAGG TGCTGACGAC CTCAACATCC | 240 |
| AGCGAGTCCT GCGGGTCAAC AGGACGCTGT TCATTGGGGA CAGGGACAAC CTCTACCGCG | 300 |
| TAGAGCTGGA GCCCCCCACG TCCACGGAGC TGCGGTACCA GAGGAAGCTG ACCTGGAGAT | 360 |
| CTAACCCCAG CGACATAAAC GTGTGTCGGA TGAAGGGCAA ACAGGAGGGC GAGTGTCGAA | 420 |
| ACTTCGTAAA GGTGCTGCTC CTTCGGGACG AGTCCACGCT CTTTGTGTGC GGTTCCAACG | 480 |
| CCTTCAACCC GGTGTGCGCC AACTACAGCA TAGACACCCT GCAGCCCGTC GGAGACAACA | 540 |
| TCAGCGGTAT GGCCCGCTGC CCGTACGACC CCAAGCACGC CAATGTTGCC CTCTTCTCTG | 600 |
| ACGGGATGCT CTTCACAGCT ACTGTTACCG ACTTCCTAGC CATTGATGCT GTCATCTACC | 660 |
| GCAGCCTCGG GGACAGGCCC ACCCTGCGCA CCGTGAAACA TGACTCCAAG TGGTTCAAAG | 720 |
| AGCCTTACTT TGTCCATGCG GTGGAGTGGG GCAGCCATGT CTACTTCTTC TTCCGGGAGA | 780 |
| TTGCGATGGA GTTTAACTAC CTGGAGAAGG TGGTGGTGTC CCGCGTGGCC CGAGTGTGCA | 840 |
| AGAACGACGT GGGAGGCTCC CCCCGCGTGC TGGAGAAGCA GTGGACGTCC TTCCTGAAGG | 900 |
| CGCGGCTCAA CTGCTCTGTA CCCGGAGACT CCCATTTCTA CTTCAACGTG CTGCAGGCTG | 960 |
| TCACGGGCGT GGTCAGCCTC GGGGGCCGGC CCGTGGTCCT GGCCGTTTTT TCCACGCCCA | 1020 |
| GCAACAGCAT CCCTGGCTCG GCTGTCTGCG CCTTTGACCT GACACAGGTG GCAGCTGTGT | 1080 |
| TTGAAGGCCG CTTCCGAGAG CAGAAGTCCC CCGAGTCCAT CTGGACGCCG GTGCCGGAGG | 1140 |
| ATCAGGTGCC TCGACCCCGG CCCGGGTGCT GCGCAGCCCC CGGGATGCAG TACAATGCCT | 1200 |
| CCAGCGCCTT GCCGGATGAC ATCCTCAACT TTGTCAAGAC CCACCCTCTG ATGGACGAGG | 1260 |
| CGGTGCCCTC GCTGGGCCAT GCGCCCTGGA TCCTGCGGAC CCTGATGAGG CACCAGCTGA | 1320 |
| CTCGAGTGGC TGTGGACGTG GGAGCCGGCC CCTGGGGCAA CCAGACCGTT GTCTTCCTGG | 1380 |
| GTTCTGAGGC GGGGACGGTC CTCAAGTTCC TCGTCCGGCC CAATGCCAGC ACCTCAGGGA | 1440 |
| CGTCTGGGCT CAGTGTCTTC CTGGAGGAGT TTGAGACCTA CCGGCCGGAC AGGTGTGGAC | 1500 |
| GGCCCGGCGG TGGCGAGACA GGGCAGCGGC TGCTGAGCTT GGAGCTGGAC GCAGCTTCGG | 1560 |
| GGGGCCTGCT GGCTGCCTTC CCCCGCTGCG TGGTCCGAGT GCCTGTGGCT CGCTGCCAGC | 1620 |
| AGTACTCGGG GTGTATGAAG AACTGTATCG GCAGTCAGGA CCCCTACTGC GGGTGGGCCC | 1680 |
| CCGACGGCTC CTGCATCTTC CTCAGCCCGG GCACCAGAGC CGCCTTTGAG CAGGACGTGT | 1740 |
| CCGGGGCCAG CACCTCAGGC TTAGGGGACT GCACAGGACT CCTGCGGGCC AGCCTCTCCG | 1800 |
| AGGACCGCGC GGGGCTGGTG TCGGTGAACC TGCTGGTAAC GTCGTCGGTG GCGGCCTTCG | 1860 |
| TGGTGGGAGC CGTGGTGTCC GGCTTCAGCG TGGGCTGGTT CGTGGGCCTC CGTGAGCGGC | 1920 |
| GGGAGCTGGC CCGGCGCAAG GACAAGGAGG CCATCCTGGC GCACGGGGCG GGCGAGGGCG | 1980 |
| TGCTGAGCGT CAGCCGCCTG GGCGAGCGCA GGGCGCAGGG TCCCGGGGGC CGGGGCGGAG | 2040 |
| GCGGTGGCGG TGGCGCCGGG GTTCCCCCGG AGGCCCTGCT GGCGCCCCTG ATGCAGAACG | 2100 |
| GCTGGGCCAA GGCCACGCTG CTGCAGGGCG CGCCCCACGA CCTGGACTCG GGGCTGCTGC | 2160 |
| CCACGCCCGA GCAGACGCCG CTGCCGCAGA AGCGCCTGCC CACTCCGCAC CCGCACCCCC | 2220 |
| ACGCCCTGGG CCCCCGCGCC TGGGACCACG GCCACCCCCT GCTCCCGGCC TCCGCTTCAT | 2280 |
| CCTCCCTCCT GCTGCTGGCG CCCGCCCGGG CCCCCGAGCA GCCCCCCGCG CCTGGGGAGC | 2340 |
| CGACCCCCGA CGGCCGCCTC TATGCTGCCC CGCCCGCCCG CGCCTCCCAC GGCGACTTCC | 2400 |
| CGCTCACCCC CCACGCCAGC CCGGACCGCC GGCGGGTGGT GTCCGCGCCC ACGGGCCCCT | 2460 |
| TGGACCCAGC CTCAGCCGCC GATGGCCTCC CGCGGCCCTG GAGCCCGCCC CCGACGGGCA | 2520 |
| GCCTGAGGAG GCCACTGGGC CCCCACGCCC CTCCGGCCGC CACCCTGCGC CGCACCCACA | 2580 |
| CGTTCAACAG CGGCCAGGCC CGGCCTGGGG ACCGCCACCG CGGCTGCCAC GCCCGGCCGG | 2640 |
| GCACAGACTT GGCCCACCTC CTCCCCTATG GGGGGGCGGA CAGGACTGCG CCCCCCGTGC | 2700 |
| CCTAGGCCGG GGGCCCCCCG ATGCCTTGGC AGTGCCAGCC ACGGGAACCA GGAGCGAGAG | 2760 |
| ACGGTGCCAG AACGCCGCGG CCCGGGGCAA CTCCGAGTGG GTGCTCAAGT CCCCCCCGCG | 2820 |
| ACCCACCCGC GGAGTGGGGG GCCCCCTCCG CCACAAGGAA GCACAACCAG CTCGCCCTCC | 2880 |
| CCCTACCCGG GGCCGCAGGA CGCTGAGACG GTTTGGGGGT GGGTGGGCGG GAGGACTTTG | 2940 |
| CTATGGATTT GAGGTTGACC TTATGCGCGT AGGTTTTGGT TTTTTTTGCA GTTTTGGTTT | 3000 |
| CTTTTGCGGT TTTCTAACCA ATTGCACAAC TCCGTTCTCG GGGTGGCGGC AGGCAGGGGA | 3060 |
| GGCTTGGACG CCGGTGGGGA ATGGGGGGCC ACAGCTGCAG ACCTAAGCCC TCCCCCACCC | 3120 |
| CTGGAAAGGT CCCTCCCCAA CCCAGGCCCC TGGCGTGTGT GGGTGTGCGT GCGTGTGCGT | 3180 |
| GCCGTGTTCG TGTGCAAGGG GCCGGGGAGG TGGGCGTGTG TGTGCGTGCC AGCGAAGGCT | 3240 |
| GCTGTGGGCG TGTGTGTCAA GTGGGCCACG CGTGCAGGGT GTGTGTCCAC GAGCGACGAT | 3300 |
| CGTGGTGGCC CCAGCGGCCT GGGCGTTGGC TGAGCCGACG CTGGGGCTTC CAGAAGGCCC | 3360 |
| GGCGGTCTCC GAGGTGCCGG TTAGGAGTTT GAACCCCCCC CACTCTGCAG AGGCAAGCGG | 3420 |
| GGACAATGCC GGGGTTTCAG GCAGGAGACA CGAGGAGGGC CTGCCCGGAA GTCACATCGG | 3480 |
| CAGCAGCTGT CTAAAGGGCT TGGGGGCCTG GGGGGCGGCG AAAG | |

Seq ID No: 167 Protein sequence:
Protein Accession #: NP_115484.1

| | |
|---|---|
| 1 11 21 31 41 51 | |
| \| \| \| \| \| \| | |
| MQTPRASPPR PALLLLLLLL GGAHGLFPED PPPLSVAPRD YLNHYPVFVG SGPGRLTPAE | 60 |
| GADDLNIQRV LRVNRTLFIG DRDNLYRVEL EPPTSTELRY QRKLTWRSNP SDINVCRMKG | 120 |
| KQECECRNFV KVLLLRDEST LFVCGSNAFN PVCANYSIDT LQPVGDNISC MARCPYDPKH | 180 |
| ANVALFSDGM LFTATVTDFL AIDAVIYRSL GDRPTLRTVK HDSKWFKEPY FVHAVEWGSH | 240 |
| VYFFFREIAM EFNYLEKVVV SRVARVCKND VGGSPRVLEK QWTSFLKARL NCSVPGDSHF | 300 |
| YFNVLQAVTG VVSLGGRPVV LAVFSTPSNS IPGSAVCAFD LTQVAAVFEG RFREQKSPES | 360 |
| IWTPVPEDQV PRPRPGCCAA PGMQYNASSA LPDDILNFVK THPLMDEAVP SLGHAPWILR | 420 |
| TLMRHQLTRV AVDVGAGPWG NQTVVFLGSE AGTVLKFLVR PNASTSGTSG LSVFLEEFET | 480 |
| YRPDRCGRPG GGETGQRLLS LELDAASGGL LAAFPRCVVR VPVARCQQYS GCMKNCIGSQ | 480 |
| DPYCGWAPDG SCIFLSPGTR AAFEQDVSGA STSGLGDCTG LLRASLSEDR AGLVSVNLLV | 600 |
| TSSVAAFVVG AVVSGFSVGW FVGLRERREL ARRKDKEAIL AHGACEAVLS VSRLGERRAQ | 660 |
| GPGGRGGGGG GGAGVPPEAL LAPLMQNGWA KATLLQGGPH DLDSGLLPTP EQTPLPQKRL | 720 |
| PTPHPHPHAL GPRAWDHGHP LLPASASSSL LLLAPARAPE QPPAPGEPTP DGRLYAARPG | 780 |
| RASHGDFPLT PHASPDRRRV VSAPTGPLDP ASAADGLPRP WSPPPTGSLR RPLGPHAPPA | 840 |
| ATLRRTHTFN SGEARPGDRH RGCHARPGTD LAHLLPYGGA DRTAPPVP | |

Seq ID NO: 168 DNA sequence:
Nucleic Acid Accession #: AW205664
Coding sequence: 1-135 (underlined sequences correspond to start and stop codons)

| | |
|---|---|
| 1 11 21 31 41 51 | |
| \| \| \| \| \| \| | |
| CGGCACGAGG AGAACAGGGG CCTCTGCCTC AGTTTGCCCG GGAGCCAGCC AGGGCCCATC | 60 |
| CTAATTTGGA GCACAGTCTT CCCGGTGCCT AGACATGCCA AGGCCCCTCC CACGTGGTAC | 120 |
| ACCCTCTCCG TTTAGTACCT GACCACCTGT TTCAAAACGC AGGTGTTTCT GGTTTAGAAA | 180 |
| CTTGGAAGGC GGAATGTGTT TTCGTGTCTT CTAGGAAGGG TCTGCTGAGG ACCAGACCAC | 240 |
| GTAAGCCTGA GTGGATCCTG ACTCAGCTGC AGCCCTTACC TGCCTCGTGC TGATGATCTA | 300 |
| TGCATGGCGT TATGTAGATC ACGTGCGGCA GAGACAGCCA CTGTCCTGTG TGCGGGTTTT | 360 |
| TAAAACAGCT GCCCTGGATG AAACGGAATA AACCAGTGAT GCTAAAAAAA AAAAAAAAAA | |

Seq ID No: 169 Protein sequence:
Protein Accession #: AW205664

1 11 21 31 41 51
| | | | | |
RHEENRGLCL SLPGSQPGPI LIWSTVFPVP RHAKAPPTWY TLSV

Seq ID NO: 170 DNA sequence:
Nucleic Acid Accession #: AB033100
Coding sequence: 32-2623 (underlined sequences correspond to start and stop codons)

| | |
|---|---|
| 1 11 21 31 41 51 | |
| \| \| \| \| \| \| | |
| AGGTCTGGGG TCCTGAGGCT GCTGGCAGAC TATGGGTACA ACGGCCAGCA CAGCCCAGCA | 60 |
| GACGGTCTCG GCAGGCACCC CATTTGAGGG CCTACAGGGC AGTGGCACGA TGGACAGTCG | 120 |
| GCACTCCGTC AGCATCCACT CCTTCCAGAG CACTAGCTTG CATAACAGCA AGGCCAAGTC | 180 |
| CATCATCCCC AACAAGGTGG CCCCTGTTGT GATCACGTAC AACTGCAAGG AGGAGTTCCA | 240 |
| GATCCATGAT GAGCTGCTCA AGGCTCATTA CACGTTGGGC CGGCTCTCGG ACAACACCCC | 300 |
| TGAGCACTAC CTGGTGCAAG GAGCTCAGGC CTTACCCCAG GGCCGCTACT TCCTGGTGCG | 360 |
| GGATGTCACT GAGAAGATGG ATGTGCTGGG CACCGTGGGA AGCTGTGGGG CCCCCAACTT | 420 |
| CCGGCAGGTG CAGGGTGGGC TCACTGTGTT CGGCATGGGA CAGCCCAGCC TCTTAGGGTT | 480 |
| CAGGCGGGTC CTCCAGAAAC TCCAGAAGGA CGGACATAGG GAGTGTGTCA TCTTCTGTGT | 540 |
| GCGGGAGGAA MCTGTGCTTT TCCTGCGTGC AGATGAGGAC TTTGTGTCCT ACACACCTCG | 600 |
| AGACAAGCAG AACCTTCATG AGAACCTCCA GGGCCTTGGA CCCGGGGTCC GGGTGGAGAG | 660 |
| CCTGGAGCTG GCCATCCGGA AAGAGATCCA CGACTTTGCC CAGCTGAGCG AGAACACATA | 720 |
| CCATGTGTAC CATAACACCG AGGACCTGTG GGGGGAGCCC CATGCTGTGG CCATCCATGG | 780 |
| TGAGGACGAC TTGCATGTGA CGGAGGAGGT GTACAAGCGG CCCCTCTTCC TGCAGCCCAC | 840 |
| CTACAGGTAC CACCGCCTGC CCCTGCCCGA GCAAGGGAGT CCCCTGGAGG CCCAGTTGGA | 900 |
| CGCCTTTGTC AGTGTTCTCC GGGAGACCCC CAGCCTGCTG CAGCTCCGTG ATGCCCACGG | 960 |
| GCCTCCCCCA GCCCTCGTCT TCAGCTGCCA GATGGGCGTG GGCAGGACCA ACCTGGGCAT | 1020 |
| GGTCCTGGGC ACCCTCATCC TGCTTCACCG CAGTGGGACC ACCTCCCAGC CAGAGGCTGC | 1080 |
| CCCCACGCAG GCCAAGCCCC TGCCTATGGA GCAGTTCCAG GTGATCCAGA GCTTTCTCCG | 1140 |
| CATGGTGCCC CAGGGAAGGA GGATGGTGGA AGAGGTGGAC AGAGCCATCA CTGCCTGTGC | 1200 |
| CGAGTTGCAT GACCTGAAAG AAGTGGTCTT GGAAAACCAG AAGAAGTTAG AAGGTATCCG | 1260 |
| ACCGGAGAGC CCAGCCCAGG GAAGCGGCAG CCGACACAGC GTCTGGCAGA GGGCGCTGTG | 1320 |
| GAGCCTGGAG CGATACTTCT ACCTGATCCT GTTTAACTAC TACCTTCATG AGCAGTACCC | 1380 |
| GCTGGCCTTT GCCCTCAGTT TCAGCCGCTG GCTGTGTGCC CACCCTGAGC TGTACCGCCT | 1440 |
| GCCCGTGACG CTGAGCTCAG CAGGCCCTGT GGCTCCGAGG GACCTCATCG CCAGGGGCTC | 1500 |
| CCTACGGGAG GACGATCTGG TCTCCCCGGA CGCGCTCAGC ACTGTCAGAG AGATGGATGT | 1560 |
| GGCCAACTTC CGGCGGGTGC CCCGCATGCC CATCTACGGC ACGGCCCAGC CCAGCGCCAA | 1620 |
| GGCCCTGGGG AGCATCCTGG CCTACCTGAC GGACGCCAAG AGGAGGCTGC GGAAGGTTGT | 1680 |
| CTGGGTGAGC CTTCGGGAGG AGGCCGTGTT GGAGTGTGAC GGGCACACCT ACAGCCTGCG | 1740 |
| GTGGCCTGGG CCCCCTGTGG CTCCTGACCA GCTGGAGACC CTGGAGGCCC AGCTGAAGGC | 1800 |
| CCATCTAAGC GAGCCTCCCC CAGGCAAGGA GGGCCCCCTG ACCTACAGGT TCCAGACCTG | 1860 |
| CCTTACCATG CAGGAGGTCT TCAGCCAGCA CCGCAGGGCC TGTCCTGGCC TCACCTACCA | 1920 |
| CCGCATCCCC ATGCCGGACT TCTGTGCCCC CCGAGAGGAG GACTTTGACC AGCTGCTGGA | 1980 |
| GGCCCTGCGG GCCGCCCTCT CCAAGGACCC AGGCACTGGC TTCGTGTTCA GCTGCCTCAG | 2040 |
| CGGCCAGGGC CGTACCACAA CTGCGATGGT GGTGGCTGTC CTGGCCTTCT GGCACATCCA | 2100 |
| AGGCTTCCCC GAGGTGGGTG AGGAGGAGCT CGTGAGTGTG CCTGATGCCA AGTTCACTAA | 2160 |
| GGGTGAATTT CAGGTAGTAA TGAAGGTGGT GCAGCTGCTA CCCGATGGGC ACCGTGTGAA | 2220 |
| GAAGGAGGTG GACGCAGCGC TGGACACTGT CAGCGAGACC ATGACGCCCA TGCACTACCA | 2280 |
| CCTGCGGGAG ATCATCATCT GCACCTACCG CCAGGCGAAG GCAGCGAAAG AGGCGCAGGA | 2340 |
| AATGCGGAGG CTGCAGCTGC GGAGCCTGCA GTACTTGGAG CGCTATGTCT GCCTGATTCT | 2400 |
| CTTCAACGCG TACCTCCACC TGGAGAAGGC CGACTCCTGG CAGAGGCCCT TCAGCACCTG | 2460 |
| GATGCAGGAG GTGGCATCGA AGGCTGGCAT CTACGAGATC CTTAACGAGC TGGGCTTCCC | 2520 |
| CGAGCTGGAG AGCGGGGAGG ACCAGCCCTT CTCCAGGCTG CGCTACCGGT CGCAGGAGCA | 2560 |
| GAGCTGCAGC CTCGAGCCCT CTGCCCCCGA GGACTTGCTG TAGGGGGCCT TACTCCCTGT | 2640 |
| GGCCCTGAGG GGTGCTCGCC TTGAAATGAT TCCCCCACTT CCTCCAGAGA CTCACCGGAG | 2760 |
| TTGGGAGCCT TTTTAGAAAG AACTTTTTAT AGGACAGGGA GACASCACAG CCATCCCTTG | 2820 |
| CAAACCACCA AGGTGTGTGG CTGACCTCCA GGGAGGAGCA CTCACTGGAG TGCTCACAAG | 2880 |
| GTGCACACTG CTGTGTGTAC CTTGCAGACA GGCCGGCGTT CAGCCTCCAA GGGGCTCACT | 2940 |
| CCCCCAGTTG CCAAACACTG TGGATCTCTC TGTCCTCTTC TCCCCTCTCT CAGATTGGCC | 3000 |
| TGGCAGCCCC TGGCACAGAG CAGACCCGGC CACTGGTAGC TCCCCACTTC CTTACTCCTG | 3060 |
| CTGCTCTGCC ATTGCCGCTC CCCTTCTTGC TGCCCAAGCA CTGCCCTCGG GCGTCTGGCA | 3120 |
| GCCTGAGGTG GGTGGAGGGG ACAGTGTTCT GGATAGATCT ATTATGTGAA AGGCAGCTTC | 3180 |
| ACCCAGTTTT CTGGACTCTC ATGCCCCCAT CTCCGACCTG GGAGACTTCA GGAATGACAA | 3240 |
| CCTACCCAGC CTGGTGGGGC TGGGAGGATG GTGGAGGTTT CTCAAGGAGC TGGAGACTTC | 3300 |
| AGGGAGCCCC TCTCATGGGG AGGAAAGAGC TTCCAGGGGG CGAACGCAGC ACAGAGGAAG | 3360 |
| AGGCCTGCTC CACTTGTCTG GGAACCTGGG CAGGAGGCAC AGAGGAAGCC AAGGCCTGGA | 3420 |
| GCTGCAGGTC CCCCGGCATC TCTCTCTGTC CCGGCAGCCC AGGATGGCCT GGTGCCCCCA | 3480 |
| CCTGCTGCAG CAGGAGCCCC AAGGAGTGCT AGCTGAGGGT GGTTGCTGGG GTGGTCCTCA | 3540 |
| TGGACAGTGA GGTGTGCAAG GGTGCACTGA GGGTGGTGGG AGGGGATCAC CTGGGTTCCA | 3600 |
| GGCCATCCTT GCTGAGCATC TTTGAGCCTG CCTTCCGGTG GGAGCAGAAA AGGCCAGACC | 3660 |
| CTGCTGAGTT AGAGGCTGCT GGGATCCACT GTTTCCACAC AGCGGGAAGG CTGCTGGGAA | 3720 |
| CAGGTGGCAG AGAAGTGCCA TGTTTGCGTT GAGCCTTGCA GCTCTTCCAG CTGGGGACTG | 3780 |
| GTGCTTGCTG AAACCCAGGA GCTGAACAGT GAGGAGGCTG TCCACCTTGC TTGGCTCACT | 3840 |
| GGGACCAGSA AAGCCTGTCT TTGGTTAGGC TCGTGTACTT CTGCAGGAAA AAAAAAAAAG | 3900 |
| GATGTGTCAT TGGTCATGAT ATTTGAAAAG GGGAGGAGGC CGAAGTTGTT CCCATTTATC | 3960 |
| CAGTATTGGA AAATATTTGA CCCCCTTGGC TGAATTCTTT TGCAGAACTA CTGTGTGTCT | 4020 |
| GTTCACTACC TTTTCAGGTT TATTGTTTTT ATTTTTGCAT GAATTAAGAC GTTTTAATTT | 4080 |
| CTTTGCAGAC AAGGTCTAGA TGCGGAGTCA GAGATGGGAC TGAATGGGGA GGGATCCTTT | 4140 |
| GTGTTCTCAT GGTTGGCTCT GACTTTCAGC TGTGTTGGGA CCACTGGCTG ATCACATCAC | 4200 |
| CTCTCTGCCT CAGTTTCCCC ATCTGTAAAA TGGGAGAATA ATACTTGCCT ACCTACCTCA | 4260 |
| CRGGGGTGTT GTGAGGATTC ATTTGTGATT TTTTTTTTTT TTTTTGTACA GAGCTTTTAA | 4320 |
| GCATTAAAAA CAGCTAAATG TG | |

Seq ID No: 171 Protein sequence:
Protein Accession #: BAA86588.1

| | |
|---|---|
| 1 11 21 31 41 51 | |
| \| \| \| \| \| \| | |
| MGTTASTAQQ TVSAGTPFEG LQGSGTMDSR HSVSIHSFQS TSLMNSKAKS IIPNKVAPVV | 60 |
| ITYNCKEEFQ IHDELLKAHY TLGRLSDNTP EHYLVQGAQA LPQGRYFLVR DVTEKNDVLG | 120 |
| TVGSCGAPNF RQVQGGLTVF GMGQPSLLGF RRVLQKLQKD GHRECVIFCV REEVLFLRAD | 180 |
| EDFVSYTPRD KQNLHENLQG LGPGVRVESL ELAIRKEIND FAQLSENTYH VYHNTEDLWG | 240 |
| EPHAVAINGE DDLHVTEEVY KRPLFLQPTY RYHRLPLPEQ GSPLEAQLDA FVSVLRETPS | 300 |
| LLQLRDAHGP PPALVESCQM GVGRTNLGMV LGTLILLERS GTTSQPEAAP TQAKPLPMEQ | 360 |
| FQVIQSFLRM VFQGRRMVEE VDRAITACAE LHDLKEVVLE NQKKLEGIRP ESPAQGSGSR | 420 |
| HSVWQRALWS LERYFYLILK NYTLEEQYPL AFALSFSRWL CAHPELYRLP VTLSSAGPVA | 480 |
| PRDLIARGSL REDDLVSPDA LSTVREMDVA NFRRVPRMPI YGTAQPSAKA LGSILAYLTD | 540 |
| AKRRLRKVVW VSLREEAVLE CUGHTYSLEW PGPPVAPDQL ETLEAQLKAH LSEPPPGKEG | 600 |
| PLTYRFQTCL TMQEVFSQHR RACPGLTYHR IPMPDFCAPR EEDFDQLLEA LRAALSKDPG | 660 |
| TGFVFSCLSG QGRTTTAMVV AVLAFWHIQG FPEVGEEELV SVPDAKFTKG EFQVVNKVVQ | 720 |
| LLPDGHRVKK EVDAALDTVS ETMTPMHYHL REIIICTYRQ AKAAKEAQEM RRLQLRSLQY | 780 |
| LERYVCLILF NAYLHLEKAD SWQRPFSTWM QEVASKAGIY EILNELGFPE LESGEDQPFS | 840 |
| RLRYRWQEQS CSLEPSAPED LL | |

Seq ID NO: 172 DNA sequence:
Nucleic Acid Accession #: AK021806.l
Coding sequence: 1-645 (underlined sequences correspond to start and stop codons)

| | |
|---|---|
| 1 11 21 31 41 51 | |
| \| \| \| \| \| \| | |
| ACTGTGCTTT TCCTGCGTGC AGATGAGGAC TTTGTGTCCT ACACACCTCG AGACAAGCAG | 60 |
| AACCTTCATG AGAACCTCCA GGGCCTTGGA CCCGGGGTCC GGGTGGAGAG CCTGGAGCTG | 120 |
| GCCATCCGGA AAGAGATCCA CGACTTTGCC CAGCTGAGCG AGAACACATA CCATGTGTAC | 180 |
| CATAACACCG AGGACCTGTG GGGGGAGCCC CATGCTGTGG CCATCCATGG TGAGGACGAC | 240 |
| TTGCATGTGA CGGAGGAGGT GTACAAGCGG CCCCTCTTCC TGCAGCCCAC CTACAGGTAC | 300 |
| CACCGCCTGC CCCTGCCCGA GCAAGGGAGT CCCCTGGAGG CCCAGTTGGA CGCCTTTGTC | 360 |
| AGTGTTCTCC GGGAGACCCC CAGCCTGCTG CAGCTCCGTG ATGCCCACGG GCCTCCCCCA | 420 |
| GCCCTCGTCT TCAGCTGCCA GATGGGCGTG GGCAGGACCA ACCTGGGCAT GGTCCTGGGC | 480 |
| ACCCTCATCC TGCTTCACCG CAGTGGGACC ACCTCCCAGC CAGAGGCTGC CCCCACGCAG | 540 |
| GCCAAGCCCC TGCCTATGGA GCAGTTCCAG GTGATCCAGA GCTTTCTCCG CATGGTGCCC | 600 |
| CAGGGAAGGA GGATGGTGGA AGAGGTGGAT AGATCTATTA TGTGAAAGGC AGCTTCACCC | 660 |
| AGTTTTCTGG ACTCTCATGC CCCCATCTCC GACCTGGGAG ACTTCAGGAA TGACAACCTA | 720 |
| CCCAGCCTGG TGGGGCTGGC AGGATGGTGG AGGTTTCTCA AGGAGCTGGA GACTTCAGGG | 780 |
| AGCCCCTCTC ATGGGGAGGA AAGAGCTTCC AGGGGGCGAA CGCAGCACAG AGGAAGAGGC | 840 |
| CTGCTCCACT TGTCTGGGAA CCTGGGCAGG AGGCACAGAG GAAGCCAAGG CCTGGAGCTG | 900 |
| CAGGTCCCCC GGCATCTCTC TCTGTCCCGG CAGCCCAGGA TGGCCTGGTG CCCCCACCTG | 960 |
| CTGCAGCACC AGCCCCAAGG AGTGCTAGCT GAGGGTGGTT GCTGGGGTGG TCCTCATGGA | 1020 |
| CAGTGAGGTG TGCAAGGGTG CACTGAGGGT GGTGGGAGGG GATCACCTGG GTTCCAGGCC | 1080 |
| ATCCTTGCTG AGCATCTTTG AGCCTGCCTT CCGGTGGGAG CAGAAAAGGC CAGACCCTGC | 1140 |
| TGAGTTAGAG GCTGCTGGGA TCCACTGTTT CCACACAGCG GGAAGGCTGC TGGGAACAGG | 1200 |
| TGGCAGAGAA GTGCCATGTT TGCGTTGAGC CTTGCAGCTC TTCCAGCTGG GGACTGGTGC | 1260 |
| TTGCTGAAAC CCAGGAGCTG AACAGTGAGG AGGCTGTCCA CCTTGCTTGG CTCACTGGGA | 1320 |
| CCAGGAAAGC CTGTCTTTGG TTAGGCTCGT GTACTTCTGC AGGAAAAAAA AAAAAGGATG | 1380 |
| TGTCATTGGT CATGATATTT GAAAAGGGGA GGAGGCCGAA GTTGTTCCCA TTTATCCAGT | 1440 |
| ATTGGAAAAT ATTTGACCCC CTTGGCTGAA TTCTTTTGCA GAACTACTGT GTGTCTGTTC | 1500 |
| ACTACCTTTT CAGGTTTATT GTTTTTATTT TTGCATGAAT TAAGACGTTT TAATTTCTTT | 1560 |
| GCAGACAAGG TCTAGATGCG GAGTCAGAGA TGGGACTGAA TGGGGAGGGA TCCTTTGTGT | 1620 |
| TCTCATGGTT GGCTCTGACT TTCAGCTGTG TTGGGACCAC TGGCTGATCA CATCACCTCT | 1680 |
| CTGCCTCAGT TTCCCCATCT GTAAAATGGG AGAATAATAC TTGCCTACCT ACCTCACGGG | 1740 |
| GGTGTTGTGA GGATTCATTT GTGATTTTTT TTTTTTTTTT TGTACAGAGC TTTTAAGCAT | 1800 |
| TAAAAACAGC TAAATGTG | |

Seq ID No: 173 Protein sequence:
Protein Accession #: AK021806.1

| | |
|---|---|
| 1 11 21 31 41 51 | |
| \| \| \| \| \| \| | |
| TVLFLRADED FVSYTPRDKQ NLHENLQGLG PCVRVESLEL AIRKEIHDFA QLSENTYHVY | 60 |
| HNTEDLWGEP HAVAINGEDD LHVTEEVYKR PLFLQPTYRY HRLPLPEQGS PLEAQLDAFV | 120 |
| SVLRETPSLL QLRDAHGPPP ALVFSCQMGV GRTNLGMVLG TLILLNRSGT TSQPEAAPTQ | 180 |
| AKPLPMEQFQ VIQSFLRMVP QGRRMVEEVD RSIM | |

Seq ID NO: 174 DNA sequence:
Nucleic Acid Accession #: NM_016580.2
Coding sequence: 1212-4766 (underlined sequences correspond to start and stop codons)

| | |
|---|---|
| 1 11 21 31 41 51 | |
| \| \| \| \| \| \| | |
| GGGAAGCGGG AGGAGAGCCA CACGGTCAAG TTGCACAGGT TCTTGCAGCT TCTGGAATCA | 60 |
| AGACCATGGG CACCCTCATA ACTCAGTGTG GGCAGGGACT GCCCCAGGGC CAATCCAAGA | 120 |
| TCCAGAGGTA GCCATAGGGT GTGACAAGTT GTGCAGATTA CAACACTCAC CCCTTGCAAT | 180 |
| AACGTCACTG CCTGTGACTC GGGGCCAGGC CCAGGCCAAA GCCCTTCCTA CATCATTTCG | 240 |
| TTTAATCCTC ACAGTTTCCT GCTGAAAGGG CTACTATTCT TACTCCCATC CCCACTCTAC | 300 |
| AGATGAGGTA ATGGAGGCCC AGGAAAGTTA AGTGACTTGT CCCAGATGAC ACCGCTGGTA | 360 |
| AGTTGCAAAG TCAGAATTTG AACTCAGGCA GTTTACCTCT GATGGCTGCT CTGTTAATCA | 420 |
| CAGCTGCTTT CCAGTGAGAC AAAAACGGGT GATCAGGGCA GAGTCAAGAC AGAGAGGTAA | 480 |
| ACAAGATTGG GAAAAAGACA GGAATGAGAG GGGAACAATG GGGGAAAAGA TAGGAACAAA | 540 |
| GAGAGTTGGG GAAGGGGAGA GAAACAGCAA ACATGACTTG CCCGGGAGGG GCATCAGTCC | 600 |
| ACGTGCAAGC AGGTGGAGGC TCAAGTTTTC TGCTCACTTG GTGATGCAGA GGCTCCCTTT | 660 |
| CCCTCAGCAG CCGCCTTGCT GCGTGGACAG CAGCTTCCCA TCTGGCCTGT CCCCGGAGCC | 720 |
| CCGGCCTCAT CCTCCTCAGC GGCAGGCCAC TTAGCTTCAC AGGAAATGCT CTTTCTCTAA | 780 |
| TTGGCATTGA AACTCACAGC CCTCCCTTTT CCTGTAGGTG GGGTTTCCAT AGGAAAAAGC | 840 |
| TGCTTCTCTG TTTCCCCAGC CTAGCAACTG TTTGGCAGTC AGAGTCCCAC ATCCTGCTCA | 900 |
| ACTGGGTCAG GTCCCTCTTA GACCAGCTCT TGTCCATCAT TTGCTGAAGT GGACCAACTA | 960 |
| GTTCCCCAGT AGGGGGTCTC CCCTGGCAAT TCTTGATCGG CGTTTGGACA TCTCAGATCG | 1020 |
| CTTCCAATGA AGATGGCCTT GCCTTGGGGT CCTGCTTGTT TCATAATCAT CTAACTATGG | 1080 |
| GACAAGGTTG TGCCGGCAGC TCTGGGGGAA GGAGCACGGG GCTGATCAAG CCATCCAGGA | 1140 |
| AACACTGGAG GACTTGTCCA GCCTTGAAAG AACTCTAGTG GTTTCTGAAT CTAGCCCACT | 1200 |
| TGGCGGTAAG CATGATGCAA CTTCTGCAAC TTCTGCTGGG GCTTTTGGGG CCAGGTGGCT | 1260 |
| ACTTATTTCT TTTAGGGGAT TGTCAGGAGG TGACCACTCT CACGGTGAAA TACCAAGTGT | 1320 |
| CAGAGGAAGT GCCATCTGGT ACAGTGATCG GGAAGCTGTC CCAGGAACTG GGCCGGGAGG | 1380 |
| AGAGGCGGAG GCAAGCTGGG GCTGCCTTCC AGGTGTTGCA GCTGCCTCAG GCGCTCCCCA | 1440 |
| TTCAGGTGGA CTCTGAGGAA GGCTTGCTCA GCACAGGCAG GCGGCTGGAT CGAGAGCAGC | 1500 |
| TGTGCCGACA GTGGGATCCC TGCCTGGTTT CCTTTGATGT GCTTGCCACA GGGGATTTGG | 1560 |
| CTCTGATCCA TGTGGAGATC CAAGTGCTGG ACATCAATGA CCACCAGCCA CGGTTTCCCA | 1620 |
| AAGGCGAGCA GGAGCTGGAA ATCTCTGAGA GCGCCTCTCT GCGAACCCGG ATCCCCCTCG | 1680 |
| ACAGAGCTCT TGACCCAGAC ACAGGCCCTA ACACCCTGCA CACCTACACT CTGTCTCCCA | 1740 |
| GTGAGCACTT TGCCTTGGAT GTCATTGTGG GCCCTGATGA GACCAAACAT GCAGAACTCA | 1800 |
| TAGTGGTGAA GGAGCTGGAC AGGGAAATCC ATTCATTTTT TGATCTGGTG TTAACTGCCT | 1860 |
| ATGACAATGG GAACCCCCCC AAGTCAGGTA CCAGCTTGGT CAAGGTCAAC GTCTTGGACT | 1920 |
| CCAATGACAA TAGCCCTGCG TTTGCTGAGA GTTCACTGGC ACTGGAAATC CAAGAAGATG | 1980 |
| CTGCACCTGG TACGCTTCTC ATAAAACTGA CCGCCACAGA CCCTGACCAA GGCCCCAATG | 2040 |
| GGGAGGTGGA GTTCTTCCTC AGTAAGCACA TGCCTCCAGA GGTGCTGGAC ACCTTCAGTA | 2100 |
| TTGATGCCAA GACAGGCCAG GTCATTCTGC GTCGACCTCT AGACTATGAA AAGAACCCTG | 2160 |
| CCTACGAGGT GGATGTTCAG GCAAGGGACC TGGGTCCCAA TCCTATCCCA GCCCATTGCA | 2220 |
| AAGTTCTCAT CAAGGTTCTG GATGTCAATG ACAACATCCC AAGCATCCAC GTCACATGGG | 2280 |
| CCTCCCAGCC ATCACTGGTG TCAGAAGCTC TTCCCAAGGA CAGTTTTATT GCTCTTGTCA | 2340 |
| TGGCAGATGA CTTGGATTCA GGACACAATG GTTTGGTCCA CTGCTGGCTG AGCCAAGAGC | 2400 |
| TGGGCCACTT CAGGCTGAAA AGAACTAATG GCAACACATA CATGTTGCTA ACCAATGCCA | 2460 |
| CACTGGACAG AGAGCAGTGG CCCAAATATA CCCTCACTCT GTTAGCCCAA GACCAAGGAC | 2520 |
| TCCAGCCCTT ATCAGCCAAG AAACAGCTCA GCATTCAGAT CAGTGACATC AACGACAATG | 2580 |
| CACCTGTGTT TGAGAAAAGC AGGTATGAAG TCTCCACGCG GGAAAACAAC TTACCCTCTC | 2640 |
| TTCACCTCAT TACCATCAAG GCTCATGATG CAGACTTGGG CATTAATGGA AAAGTCTCAT | 2700 |
| ACCGCATCCA GGACTCCCCA GTTGCTCACT TAGTAGCTAT TGACTCCAAC ACAGGAGAGG | 2760 |
| TCACTGCTCA GAGGTCACTG AACTATGAAG AGATGGCCGG CTTTGAGTTC CAGGTGATCG | 2820 |
| CAGAGGACAG CGGCCAACCC ATGCTTGCAT CCAGTGTCTC TGTGTGGGTC AGCCTCTTGG | 2880 |
| ATGCCAATGA TAATGCCCCA GAGGTGGTCC AGCCTGTGCT CAGCGATGGA AAAGCCAGCC | 2940 |
| TCTCCGTGCT TGTGAATGCC TCCACAGGCC ACCTGCTGGT GCCCATCGAG ACTCCCAATG | 3000 |
| GCTTGGGCCC AGCGGGCACT GACACACCTC CACTGGCCAC TCACAGCTCC CGGCCATTCC | 3060 |
| TTTTGACAAC CATTGTGGCA AGAGATGCAG ACTCGGGGGC AAATGGAGAG CCCCTCTACA | 3120 |
| GCATCCGCAG TGGAAATGAA GCCCACCTCT TCATCCTCAA CCCTCATACG GGGCAGCTGT | 3180 |
| TCGTCAATGT CACCAATGCC AGCAGCCTCA TTGGGAGTGA GTGGGAGCTG GAGATAGTAG | 3240 |
| TAGAGGACCA GGGAAGCCCC CCCTTACAGA CCCGAGCCCT GTTGAGGGTC ATGTTTGTCA | 3300 |
| CCAGTGTGGA CCACCTGAGG GACTCAGCCC GCAAGCCTGG GGCCTTGAGC ATGTCGATGC | 3360 |
| TGACGGTGAT CTGCCTGGCT GTACTGTTGG GCATCTTCGG GTTGATCCTG GCTTTGTTCA | 3420 |
| TGTCCATCTG CCGGACAGAA AAGAAGGACA ACAGGGCCTA CAACTGTCGG GAGGCCGAGT | 3480 |
| CCACCTACCG CCAGCAGCCC AAGAGGCCCC AGAAACACAT TCAGAAGGCA GACATCCACC | 3540 |
| TCGTGCCTGT GCTCAGGGGT CAGGCAGGTG AGCCTTGTGA AGTCGGGCAG TCCCACAAAG | 3600 |
| ATGTGGACAA GGAGGCGATG ATGGAAGCAG GCTGGGACCC CTGCCTGCAG GCCCCCTTCC | 3660 |
| ACCTCACCCC GACCCTGTAC AGGACGCTGC GTAATCAAGG CAACCAGGGA GCACCGGCGG | 3720 |
| AGAGCCGAGA GGTGCTGCAA GACACGGTCA ACCTCCTTTT CAACCATCCC AGGCAGAGGA | 3780 |
| ATGCCTCCCG GGAGAACCTG AACCTTCCCG AGCCCCAGCC TGCCACAGGC CAGCCACGTT | 3840 |
| CCAGGCCTCT GAAGGTTGCA GGCAGCCCCA CAGGGAGGCT GGCTGGAGAC CAGGGCAGTG | 3900 |
| AGGAAGCCCC ACAGAGGCCA CCAGCCTCCT CTGCAACCCT GAGACGGCAG CGACATCTCA | 3960 |
| ATGGCAAAGT GTCCCCTGAG AAAGAATCAG GGCCCCGTCA GATCCTGCGG AGCCTGGTCC | 4020 |
| GGCTGTCTGT GGCTGCCTTC GCCGAGCGGA ACCCCGTGGA GGAGCTCACT GTGGATTCTC | 4080 |
| CTCCTGTTCA GCAAATCTCC CAGCTGCTGT CCTTGCTGCA TCAGGGCCAA TTCCAGCCCA | 4140 |
| AACCAAACCA CCGAGGAAAT AAGTACTTGG CCAAGCCAGG AGGCAGCAGG AGTGCAATCC | 4200 |
| CAGACACAGA TGGCCCAAGT GCAAGGGCTG GAGGCCAGAC AGACCCAGAA CAGGAGGAAG | 4260 |
| GGCCTTTGGA TCCTGAAGAG GACCTCTCTG TGAAGCAACT GCTAGAAGAA GAGCTGTCAA | 4320 |
| GTCTGCTGGA CCCCAGCACA GGTCTGGCCC TGGACCGGCT GAGCGCCCCT GACCCGGCCT | 4380 |
| GGATGGCGAG ACTCTCTTTG CCCCTCACCA CCAACTACCG TGACAATGTG ATCTCCCCGG | 4440 |
| ATGCTGCAGC CACGGAGGAG CCAAGGACCT TCCAGACGTT CGGCAAGGCA GAGGCACCAG | 4500 |
| AGCTGAGCCC AACAGGCACG AGGCTGGCCA GCACCTTTGT CTCGGAGATG AGCTCACTGC | 4560 |
| TGGAGATGCT GCTGGAACAG CGCTCCAGCA TGCCCGTGGA GGCCGCCTCC GAGGCGCTGC | 4620 |
| GGCGGCTCTC GGTCTGCGGG AGGACCCTCA GTTTAGACTT GGCCACCAGT GCAGCCTCAG | 4680 |
| GCATGAAAGT GCAAGGGGAC CCAGGTGGAA AGACGGGGAC TGAGGGCAAG AGCAGAGGCA | 4740 |
| GCAGCAGCAG CAGCAGGTGC CTGTGAACAT ACCTCAGACG CCTCTGGATC CAAGAACCAG | 4800 |
| GGGCCTGAGG ATCTGTGGAC AAGAGCTGGT TTCTAAAATC TTGTAACTCA CTAGCTAGCG | 4860 |
| GCGGCCTGAG AACTTTAGGG TGACTGATGC TACCCCCACA GAGGAGGCAA GAGCCCCAGG | 4920 |
| ACTAACAGCT GACTGACCAA AGCAGCCCCT TGTAAGCAGC TCTGAGTCTT TTGGAGGACA | 4980 |
| GGGACGGTTT GTGGCTGAGA TAAGTGTTTC CTGGCAAAAC ATATGTGGAG CACAAAGGGT | 5040 |
| CAGTCCTCTG GCAGAACAGA TGCCACGGAG TATCACAGGC AGGAAAGGGT GGCCTTCTTG | 5100 |
| GGTAGCAGGA GTCAGGGGGC TGTACCCTGG GGGTGCCAGG AAATGCTCTC TGACCTATCA | 5160 |
| ATAAAGGAAA AGCAGTGATT CAAAAAAAAA AAAAAAAAAA AAAAAAAAAA | |

Seq ID No: 175 Protein sequence:
Protein Accession #: NP_057664.1

| | |
|---|---|
| 1 11 21 31 41 51 | |
| \| \| \| \| \| \| | |
| MMQLLQLLLG LLGPGGYLFL LGDCQEVTTL TVKYQVSEEV PSGTVIGKLS QSLGREERRR | 60 |
| QAGAAFQVLQ LPQALPTQVD SEEGTLSTGR RLDREQLCRQ WDPCLVSFDV LATGDLALIN | 120 |
| VEIQVLDIND IQPRFPKGEQ ELEISESASL RTRIPLDRAL DPDTGPNTLN TYTLSPSEHF | 180 |
| ALDVIVGPDE TKNAELIVVK ELDREIHSFF DLVLTAYDNG NPPKSGTSLV KVNVLDSNDN | 240 |
| SPAFAESSLA LEIQEDAAPG TLLIKLTATD PDQGPNGEVE FFLSKHNPPE VLDTFSIDAK | 300 |
| TGQVILRRPL DYEKNPAYEV DVQARDLGPN PIPAHCKVLI KVLDVNDNIP SIHVTWASQP | 360 |
| SLVSEALPKD SFIALVMADD LDSGHNGLVN CWLSQELGHF RLKRTNGNTY MLLTNATLDR | 420 |
| EQWPKYTLTL LAQDQGLQPL SAKKQLSIQI SDINDNAPVF EKSRYEVSTR ENNLPSLNLI | 480 |
| TIKAHDADLG INGKVSYRIQ DSPVAHLVAI DSNTGEVTAQ RSLNYEEMAG FSFQVIAEDS | 540 |
| GQPMLASSVS VWVSLLDAND NAPEVVQPVL SDGKASLSVL VNASTGHLLV PTETPNGLGP | 600 |
| AGTDTPPLAT HSSRPFLLTT IVARDADSGA NGEPLYSTRS GNEAHLFILN PHTGQLFVNV | 660 |
| TNASSLIGSE WELEIVVEDQ GSPPLQTRAL LRVMFVTSVD HLRDSARKPG ALSMSMLTVI | 720 |
| CLAVLLGIFG LILALFMSIC RTEKKDNRAY NCREAESTYR QQPKRPQKHI QKADINLVPV | 780 |
| LRGQAGEPCE VGQSHKDVDK EAMMEAGWDP CLQAPFHLTP TLYRTLRNQG NQGAPAESRE | 840 |
| VLQDTVNLLF NHPRQRNASR ENLNLPEPQP ATGQPRSRPL KVAGSPTGRL AGDQGSEEAP | 900 |
| QRPPASSATL RRQRHLNGKV SPEKESGPRQ ILRSLVRLSV AAFAERNPVE ELTVDSPPVQ | 960 |
| QISQLLSLLH QGQFQPKPNH RGNKYLAKPG GSRSAIPDTD GPSARAGGQT DPEQEEGPLD | 1020 |
| PEEDLSVKQL LEEELSSLLD PSTGLALDRL SAPDPAWMAR LSLPLTTNYR DNVISPDAAA | 1080 |
| TEEPRTFQTF GKAEAPELSP TGTRLASTFV SEMSSLLEML LEQRSSMPVE AASEALRRLS | 1140 |
| VCGRTLSLDL ATSAASGMKV QGDPGGKTGT EGKSRCSSSS SRCL | |

Seq ID NO: 176 DNA sequence:
Nucleic Acid Accession #: AL109712.1
Coding sequence: 2-128 (underlined sequences correspond to start and stop codons)

| | |
|---|---|
| 1 11 21 31 41 51 | |
| \| \| \| \| \| \| | |
| GAGTCTCTTT GGGCCAGCCG GGCTGCTGCA GACAGACAGG AAGCACGCCT GACGCTCCTC | 60 |
| TACCCTCGGG CAGCACAGCG GGGCTGGGAC TCACTCTAGC TTGCCCAGCA ACTTGCTTTC | 120 |
| CTGTGTGAAC TCTGGCAGCC TGCCCTCTCT GTGCAAAGCT GCCACTGGGG CCTGCTCAGG | 180 |
| GTGGCCTGGA ACTTGGAGGT GGGCAGTCAG GGCCTAGGAT GGGCCTGTGT CACCAGGGCA | 240 |
| TGTGCCCTTG GGCCAGTTAC TTCCTCTCAG AGCCTTGGGC TCCTCCTCTG AGGATGGGGC | 300 |
| TTGTTGGTGT GAAATGAGGT GAGCATGTTG AGTTGGGGAG CAGCAGGACA CGCACCTGCA | 360 |
| GGCAGCCGCC CTGGCCACGC TCCCTCCCTA CCTTCCGAGT CCTGGGACAG ACACAGTAGA | 420 |
| GCACAGCGGG CCAGCCTGCT CTCTTCTCTG TCTACTTTTT GCAGAAGAGT CAACAGATAC | 480 |
| AACAGGCCCA GGGAGGTGCC CCTGGGGGCC CCAGTCCCCA TCACTCCAAC GGGCAGTCCT | 540 |
| GCAAGTGACA AGGTGGGCCC AATCCCTGTG GAACAGGTCT CTGAGGACCA CAGAGTGGGG | 600 |
| CCCCAGGGAA AGCTGGGAGC CGAGCTAGAG GCAGGCAGCA AGTAAGGGCA AAGCTGTGCC | 660 |
| CCTGCCCGGA AGACCTTCCT GCCCCCAGAA CCCGACCCTC CGCAGATAGC CCTCCCTGGG | 720 |
| CAGCAGCCCC CCAGCTTCCA AGGCCCGTGC CTCACCAGAC GCCATGCTCT CACGGACTTG | 780 |
| TTTGCTGCTC TGTACCCTGC AGATCTGCCC CAGAGGAGCA GGTGAAAAGC CGCGCCTGCC | 840 |
| GAGGTGCTGT GGCGGTGGAG TTTTGGGCAG AGGAGTGGGG GGAAGAGTTT CTCACTTTTA | 900 |
| AGATTCTCCA AATCCAAGAT GAAGTCATGC TGTGCTTTGG AATGGTAGAT GCTCATTTAT | 960 |
| GTAAAATCAT AATAAATGTT ACACAAACTG TTAAAAAAAA AAAAAAAAAA AAAAAA | |

Seq ID NO: 177 Protein sequence:
Protein Accession #: AL109712.1

1 11 21 31 41 51
| | | | | |
VSLGQPCCCR QTGSTPDAPL PSGSTAGLGL TLACPATCFP V

Seq ID NO: 178 DNA sequence:
Nucleic Acid Accession #: none found
Coding sequence: 3-107 (underlined sequences correspond to start and stop codons)

| | |
|---|---|
| 1 11 21 31 41 51 | |
| \| \| \| \| \| \| | |
| AATGGAGCAC TCCAAAGAAC GATTTGACCA ATAGCATTTC TTCTCTGGGG GTTGTATTTC | 60 |
| AAAGCATGCA ACTCTCCAGG GAACCAGAAC TAAATTGCTT AAAATGAAGT CATTCCTCAG | 120 |
| ATTAACTTCC TCAGATAAAG TGTCAGCGGT CTGCAGAAAC GAAGAAGACA AAACTGAGAT | 180 |
| TATCACTCAT AATTCTCTTA CTTACTATGT CAGTGAAACA ATGACTTTGC ATTTTTGCAA | 240 |
| TCCTAGAACA TTCTTCATTA GCCCTGGGTC ATGACCTCTT CCAGTTAATT CTCTTTCACA | 300 |
| CCTTTAGGAA AGATTTAAGA TGAACCTTCA ATAGGATATT AACATAACTC ATAGCCAATA | 360 |
| CCACAGCTGC CTTTCAAATT AATGAGGTTA ATTGTTCTCC AGCAAACATG AGTTTGTCTT | 420 |
| TGGCATTTTA AATGCTTCCC ATTGATCTGA CATTTTGCTG TTTCAAGTTT TAAAGGGCTC | 480 |
| AAATCAAAGA CTATTGATAA CTGAGCAAAG AGCGAAGATC CAGAAATACG AAAACATTGT | 540 |
| CTTTTTTTTT CCATGAAAAA CAATCATAGC CTTTTGAATT CAATCGAAGT TTCTACATTA | 600 |
| GCCATCTAAG ACTTATTTAA TTATTTCTGT TCTCAGTCAA GCTAATTCAA GTGAATGAAC | 660 |
| AGTATTGACT TTTAAAATCT TTTTTAAATT TTTTTAAATC TTTAGTTTAT TAAGTTTGTA | 720 |
| GAAAAGCTCT GGGGCCATGA CCACTTACGT AAATGTTTCA GTTTAAAAAC AAAAGATTCA | 780 |
| GGCCTCTAAT TTGAGCCAAA TCCAGGTGAT CTTGTTTGAA ATTTTTGATG AATTTGAAAA | 840 |
| GATGAAAGTG GAACTTTTAA CATTCATGTT CCCCAAATTT TTCACTGGGA AGGGATGCTA | 900 |
| ATTGCCTACT TAAGATATAA GTTCAAGAAT AACATTTTCA TACAAAATTC AGAAAACTGC | 960 |
| TTGACACAGC AGTGACATAG TTAGATGTGG CTCAGATGCC TTCCAAACCT GAGGGTCCCC | 1020 |
| AAAGATTTCT TTACCAGTTG TTTTTAACTA TGAATCTTAA TCTTGTTCAT TCCCCTGCCA | 1080 |
| AAACAAATTT AAAAG | |

Seq ID No: 179 Protein sequence:
Protein Accession #: none found

1 11 21 31 41 51
| | | | | |
WSTPKNDLTN SISSLGVVFQ SMQLSREPEL NCLK

Seq ID NO: 180 DNA sequence:
Nucleic Acid Accession #: none found
Coding sequence: 2-176 (underlined sequences correspond to start and stop codons)

| | |
|---|---|
| 1 11 21 31 41 51 | |
| \| \| \| \| \| \| | |
| CCGGGTGGGG CCTCGGGATG CAGGCGCCGG TGCCCGCCCC CCTGGGCCTG CTGGACCCCG | 60 |
| CACAAGGGCT TTCGAGGAGG AAGAAGACGT CGCTCTGGTT TGTGCGGTCT CTGCTGCTCG | 120 |
| TGTCCGTCCT CATAGTCACC CTCGGGCTGG CTGCATCAGC AGGACGGAGA ATGTGACCGT | 180 |
| TGGGGGCTAC TACCCAGGGA TCATTCTCGG CTTTGGATCT TTCTTAGGAA TTATTGGCAT | 240 |
| CAACTTGGTC GACAATACAA GGCAAATGCT CCTGCCAGCC ATCGTGTTTA TCAGTTTTGG | 300 |
| CGTGGTGGCC GCCTTCTGCT GCGCCATCGT GGACGGCGTA TTTGCACCAC AGCACATTGA | 360 |
| ACCGAGGCCC CTCACCACGG GAAGATGCCA GTTTTACTCC AGTGGGGTGG GGTACTTGTA | 420 |
| CGATGTCTAC CAGACAGAGG TGAGCAGGAG CACTGAGATT CATGTGGCTT TTGCTCAGCT | 480 |
| AACCCCGCCG ACCCCACGCG GTTTTCCCTG CACATAGGCG TGCTCTGAAT ATTTGGATTC | 540 |
| TAATAGTTCC TGGGGGTCAC CCCTGCAGCT GGTGAACCGT TGATGCCCCC TGTGTAAGGG | 600 |
| ACCTTGACAT TTCGATGTGC TGTATTTCAC TCTGGAGTCA CAGTTCTCGA CTTGCTTCAT | 660 |
| TAAATCACAA CAGTCTCAGA AAACAACCGC ACCACCCCGC AATCCCACCA AAGGGCCGCG | 720 |
| CCGTCCCTAA GAGTTATCCC | |

Seq ID No: 181 Protein sequence:
Protein Accession #: none found

1 11 21 31 41 51
| | | | | |
RVCPRDAGAG ARAPGPAGPR RRAFEEEEDV ALVCGVSAAG VRPHSHRRAG CISRTENV

Seq ID NO: 182 DNA sequence:
Nucleic Acid Accession #: AK001579.1
Coding sequence: 1150-2637 (underlined sequences correspond to start and stop codons)

| | |
|---|---|
| 1 11 21 31 41 51 | |
| \| \| \| \| \| \| | |
| TTTTCTCTGC TTTTCGCTAC CCCGGTCACT CTCATTTCTC TCCCCTATTC CTTGTCTCTT | 60 |
| CCCCCATCCC CCTTTCTCCT GTCCTCCCCC TGCCTCTACA GTGGTTCTCC CCGCTGAGCT | 120 |
| GCCACCAGCT GCTGGGCCCC GGGCTGCTGC GGCTGGGCCG CCTATGGCTG CGGTCCCCCT | 180 |
| CCCATACAGC CCCGGCCCCT GGTCTCTGGC TGTCAGGGTT TGGCCTCCTT CGTGGTGACC | 240 |
| ACCTCTTCCT GTGCTCAGCG CCGGGCCCAG GCCCCCCAGC CCCTGAGGAC ATGGTGCATC | 300 |
| TGCGGCGGCT ACAGGAGATC AGTGTGGTTT CTGCAGCTGA CACCCCAGAT AAGAAAGAGC | 360 |
| ATTTGGTCCT GGTGGAGACA GGAAGGACCC TGTATCTGCA AGGAGAGGGC CGGCTGGACT | 420 |
| TCACGGCATG GAACGCAGCC ATTGGGGGCG CGGCTGGTGG GGGCGGCACA GGGCTGCAGG | 480 |
| AGCAGCAGAT GAGCCGGGGT GACATCCCCA TCATCGTGGA TGCCTGCATC AGTTTTGTTA | 540 |
| CCCAGCATGG GCTCCGGCTG GAAGGTGTAT ACCGGAAAGG GGGCGCTCGT GCCCGCAGCC | 600 |
| TGAGACTCCT GGCTGAGTTC CGTCGGGATG CCCGGTCGGT GAAGCTCCGA CCAGGGGAGC | 660 |
| ACTTTGTGGA GGATGTCACT GACACACTCA AACGCTTCTT TCGTGAGCTC GATGACCCTG | 720 |
| TGACCTCTGC ACGGTTGCTG CCTCGCTGGA GGGAGGCTGC TGGTATTCCT AACATCCCTG | 780 |
| AGAGCCAAGG CCCAACCAGG ATCTCTGCCT TCCCCCACCA GAATCCATGG TTTGGCAGCC | 840 |
| CTCCGCCCCA TCACTTCCCA CCCTGGGGGA TCATCCAGAG ACTTGGCTCA GGGGGAGGTG | 900 |
| GGAAGGGGGC AGAGACACAT CCATCCTGCA TTTGTGCCTA AAAATCCCTC CCTCTGTACC | 960 |
| AGCTGCCACT CTTTCTTCCC GGGTCCTCCC CAACCCTCCT CCATTCCATC CCCAGAGCTG | 1020 |
| CCCCAGAAGA ATCAGCGCCT GGAGAAATAT AAAGATGTGA TTGGCTGCCT GCCGCGGGTC | 1080 |
| AACCGCCGCA CACTGGCCAC CCTCATTGGG CATCTCTATC GGGTGCAGAA ATGTGCGGCT | 1140 |
| CTAAACCAGA TGTGCACGCG GAACTTGGCT CTGCTGTTTG CACCCAGCGT GTTCCAGACG | 1200 |
| GATGGGCGAG GGGAGCACGA GGTGCGAGTG CTGCAAGAGC TCATTGATGG CTACATCTCT | 1260 |
| GTCTTTGATA TCGATTCTGA CCAGGTAGCT CAGATTGACT TGGAGGTCAG TCTTATCACC | 1320 |
| ACCTGGAAGG ACGTGCAGCT GTCTCAGGCT GGAGACCTCA TCATGGAAGT TTATATAGAG | 1380 |
| CAGCAGCTCC CAGACAACTG TGTCACCCTG AAGGTGTCCC CAACCCTGAC TGCTGAGGAG | 1440 |
| CTGACTAACC AGGTACTGGA GATCGGGGGG ACAGCAGCTG GGATGGACTT GTGGGTGACT | 1500 |
| TTTGAGATTC GCGAGCATGG GGAGCTGGAG CGGCCACTGC ATCCCAAGGA AAAGGTCTTA | 1560 |
| GAGCAGGCTT TACAATGGTG CCAGCTCCCA GAGCCCTgCT CAGCTTCCCT GCTCTTGAAA | 1620 |
| AAAGTCCCCC TGGCCCAAGC TGGCTGCCTC TTCACAGGTA TCCGACGTGA GAGCCCACGG | 1680 |
| GTGGGGCTGT TGCGGTGTCG TGAGGAGCCA CCTCGCTTGC TGGGAAGCCG CTTCCAGGAG | 1740 |
| AGGTTCTTTC TGCTGCGTGG CCGCTGCCTG CTGCTGCTCA AGGAGAAGAA AAGCTCTAAA | 1800 |
| CCAGAACGGG AGTGGCCTTT GGAAGGTGCC AAGGTCTACC TGGGAATCCG CAAGAAGTTA | 1860 |
| AAGCCCCCAA CACCGTGGGG CTTCACATTG ATACTAGAGA AGATGCACCT CTACTTGTCC | 1920 |
| TGCACTGACG AGGATGAAAT GTGGGATTGG ACCACCAGCA TCCTTAAAGC CCAGCACGAT | 1980 |
| GACCAGCAGC CAGTGGTCTT ACGACGCCAT TCCTCCTCTG ACCTTGCCCG TCAGAAGTTT | 2040 |
| GGCACTATGC CTTTGCTGCC TATCCGTGGG GATGACAGTG GAGCCACCCT CCTCTCTGCC | 2100 |
| AATCAGACCC TGCGGCGACT ACACAACCGG AGGACCCTGT CCATGTTCTT TCCAATGAAG | 2160 |
| TCATCCCAGG GGTCTGTGGA GGAGCAAGAG GAGCTGGAGG AGCCTGTGTA CGAGGAGCCA | 2220 |
| GTGTATGAGG AAGTAGGGGC CTTCCCTGAG TTGATCCAGG ACACTTCTAC CTCCTTCTCC | 2280 |
| ACCACACGGG AGTGGACAGT GAAGCCAGAG AACCCCCTCA CCAGCCAGAA GTCATTGGAT | 2340 |
| CAACCCTTTC TCTCCAAGTC AAGCACCCTT GGCCAGGAGG AGAGGCCACC TGAGCCCCCT | 2400 |
| CCAGGCCCCC CTTCAAAGAG CAGTCCCCAG GCACGGGGGT CCCTAGAGGA ACAGCTGCTC | 2460 |
| CAGGAGCTCA GCAGCCTCAT CCTGAGGAAA GGAGAGACCA CTGCAGGCCT GGGAAGTCCT | 2520 |
| TCCCAGCCAT CCAGCCCCCA ATCCCCCAGC CCCACTGGCC TTCCAACACA GACACCTGGC | 2580 |
| TTCCCCACCC AACCCCCATG CACTTCCAGT CCACCCTCCA GCCAGCCCCT CACATGACCC | 2640 |
| TAGGACCAGC AGTCTGAGAG GGTAGGTACC AGAAGACCCA GAAACTCTTA TCGTGGCACT | 2700 |
| GTTGCAGCTT CCTCTGCCCT GGCTGGAAAG ACTCCAGAAT CCAGTGTGGT GCTGTGGAAG | 2760 |
| GAGCACTGGA CTAAAGGCTT CAGTGGCTGC GTGTCCCAGG ACAGGTCATG GCCCCTCTCT | 2820 |
| GGGCCCAGCC CATTTATCTA TACCATGAGG TAACTGAAGT AAGGAGAGCA GTGAATGTCA | 2880 |
| AACTGTGTTT CTTAGAGCCA TAAGCCCCAC ATATTATCCC TGAACAAGGG CAGCTCCTGC | 2940 |
| TTTATATATT TGATACGTAG GGGTTCCATG AGAGATTTTG GGTTTTAAAG GAATGGTTTT | 3000 |
| ACTGCATTAA AGAAAAAAAA TGCTTTGGAA ACCAGAGGCC TGGGTGATGT TAAAGTCTAT | 3060 |
| CCTGTCCCAC TTCCTACATT CTGGGACTAC CGTGAAGCCT GGAGTAGGGA GAGCGAGTTT | 3120 |
| GGGAGCTGCG ACTCGGGGAG TCAAAAATAG ATGAGTAATT GTCAATAAAC CTGGGAACC | |

Seq ID No: 183 Protein sequence:
Protein Accession #: AK001579.1

| | |
|---|---|
| 1 11 21 31 41 51 | |
| \| \| \| \| \| \| | |
| MSLTESNASF VSSMTLPLHG CCLAGGRLLV FLRSLRAKAQ PCSLPSPTRI HGLAALRPIT | 60 |
| SHPGGSSRDL AQGEVGRGQR HIHPAFVPKN PSLCTSCNSF FPGPPQPSSI PSPELPQKNQ | 120 |
| RLEKYKDVIG CLPRVNRRTL ATLIGHLYRV QKCAALNQMC TRNLALLFAP SVFQTDGRGE | 180 |
| HEVRVLQELI DGYISVFDID SDQVAQIDLE VSLITTWKDV QLSQAGDLIM EVYIEQQLPD | 240 |
| NCVTLKVSPT LTAEEILNQV LEMRGTAAGM DLWVTFEIRE EGELERPLHP KEKVLEQALQ | 300 |
| WCQLPEPCSA SLLLKKVPLA QAGCLFTGTR RESPRVGLLR CREEPPRLLG SRFQERFFLL | 360 |
| RGRCLLLLKE KKSSKPEREW PLEGAKVYLG IRKKLKPPTP WGFTLILEKM NLYLSCTDED | 420 |
| EMWDWTTSIL KAQNDDQQPV VLRRHSSSDL ARQKFGTMPL LPIRGDDSGA TLLSANQTLR | 480 |
| RLHNRRTLSM FFPMKSSQGS VEEQEELEEP VYEEPVYEEV GAFPELIQDT STSFSTTREW | 540 |
| TVKPENPLTS QKSLDQPFLS KSSTLGQEER PPEPPPGPPS KSSPQARGSL EEQLLQELSS | 600 |
| LILRKGETTA GLGSPSQPSS PQSPSPTGLP TQTPGFPTQP PCTSSPPSSQ PLT | |

Seq ID NO: 184 DNA sequence:
Nucleic Acid Accession #: none found
Coding sequence: 1-81 (underlined sequences correspond to start and stop codons)

| | |
|---|---|
| 1 11 21 31 41 51 | |
| \| \| \| \| \| \| | |
| GTAGAGTTAG TGTCAATGTG CTTAGAATAT ACCAAATTCA TAAACATTTT CTCTAAAAAA | 60 |
| GTATTAAGCT TAAAAAGTTA ATTCAGTTTA AGGAATATAA ACCAAATTAT TTTATATTTG | 120 |
| AATCTCAACA TAAGAAGTCA AAATGTAATG CTGCCAGATA ACAATATCAA AGGTATTTTT | 180 |
| CTTTCTCTAT AATTTCATCA GTATGTCCTC TCCCTTTTCT CCTATTTGTC AAATTTTAGC | 240 |
| AACCCTAACT CTGCTAATTA TAAGCTAGGC AAGTAATCTT GGACAAGTTA TTTGACCTCT | 300 |
| CACTGCACCA GCTTTGTTAT CTGTAAAATG ATGATAATAC CAACACCTTC TTCTTGGGGT | 360 |
| ACTGAAGATG AGAGAACATG ATATGTGTAA AGTGCCTTCC ACAATACCCA GAACATAGCA | 420 |
| AACATGTAAT GAATGTAGTA ATAGTAATTA TTTTATTTTC TTTTGATTCA GTTGGGACTA | 480 |
| TGTTCAGCTG TAACAGAATA CCCAAAATAA CAGTTTTAAA CAAATTAAAG TTTTGTTGTG | 540 |
| AAGTTTTGTT ACGAATTCAG ACAATCCAGG GCTTTTATAG ATGCACCAGG ATCAGCAGGT | 600 |
| ACAAAGGCAT CTTTCCTGAT TTCTGCCAGT CTCAATGCAT GGGTTGCAAT CCAGAGTCCA | 660 |
| GGATGGCAGT TCCAGCCCTG GTTACGCCCA TATTAGCACA CAGAAAGAAA GAGAAAGGGA | 720 |
| TGTGCCTCTT CACTTTAATC ATAGCTCCCA CTAGATGCAC CCACTACTTC TGCTGATACT | 780 |
| CCATTAGCTA ATGCTTGCTT ACATGGTCAC ACTTAGTTTC CAGAGAGACA TGTGTGGACA | 840 |
| GTCATGTGCT CAATTAATAT CCAAGTGTCC AATTACTGAG AAAAAAAGAA ACTAGCACCT | 900 |
| TTGCTTGGTT GCATTCTTCT TAGCATAAGC CACATTCTTT TTATGAAGTT GTCCTCAGTT | 960 |
| ACTTGGATGC CTCAGTTGTC CTTTCATTTA GAAATGCTCC TTGGACATCC TGAATCTGAC | 1020 |
| TTCTTTTGTC ATCAGCACCA TCACTACCAC TGCCTTCTTC AAAGCCACCA CGTTCTGTCC | 1080 |
| CAGGATGGTT GCAACAACCA CCATAGGGAC TTTTTGCTTC TACTTCCACA CAATAGCCAG | 1140 |
| AGTAAGCTTT TGAAAATGTA GGTCAGATCA TGTCTCTCTC TTCTCTTCAA AACCCTCCGA | 1200 |
| TGGCTTTTCA TATTACTCAA AAGAAAACCT AAAACTTTGC TGTGAGATCT ATGTGACCCG | 1260 |
| GCTTATTCTT CCTCTTACTT TATCTCTGTA TTGCTCTTCC TCACTCTACT CCAGCCATCC | 1320 |
| CACCTCCTTG CTGCTTGTCC TATACTCCTA AAAGAAGTTC AGTCTTCCCT TATGATATTT | 1380 |
| GCACTTAAAA TAGAAAAAAA AAAAAAAAAA AGCTCAGAGA GGCTGAGTTG TCCAAGGTCA | 1440 |
| TGCAGGTTAG AAGTCATGGA GCTGGGATCT AAATCCATGT CAGTCTGACT ATGAGTTCTG | 1500 |
| CACCGTTCTA TTCAACCCCA TTGCCTAGAG GTGCTTGATT GCTCAATAAT AGATTCCATG | 1560 |
| GACACAGTCA GCTCTTTCTG AGAAAAGGCA GCTCAGCATT TCCATGAGAT CCGCACATCC | 1620 |
| TTTTGCAGAA GAAAAC | |

Seq ID No: 185 Protein sequence:
Protein Accession #: none found

1 11 21 31 41 51
| | | | | |
VELVSMCLEY TKFINIFSKK VLSLKS

Seq ID NO: 186 DNA sequence:
Nucleic Acid Accession #: NM_002203.2
Coding sequence: 43-3588 (underlined sequences correspond to start and stop codons)

| | |
|---|---|
| 1 11 21 31 41 51 | |
| \| \| \| \| \| \| | |
| CTGCAAACCC AGCGCAACTA CGGTCCCCCG GTCAGACCCA GGATGGGGCC AGAACGGACA | 60 |
| GGGGCCGCGC CGCTGCCGCT GCTGCTGGTG TTAGCGCTCA GTCAAGGCAT TTTAAATTGT | 120 |
| TGTTTGGCCT ACAATGTTGG TCTCCCAGAA GCAAAAATAT TTTCCGGTCC TTCAAGTGAA | 180 |
| CAGTTTGGGT ATGCAGTGCA GCAGTTTATA AATCCAAAAG GCAACTGGTT ACTGGTTGGT | 240 |
| TCACCCTGGA GTGGCTTTCC TGAGAACCGA ATGGGAGATG TGTATAAATG TCCTGTTGAC | 300 |
| CTATCCACTG CCACATGTGA AAAACTAAAT TTGCAAACTT CAACAAGCAT TCCAAATGTT | 360 |
| ACTGAGATGA AAACCAACAT GAGCCTCGGC TTGATCCTCA CCAGGAACAT GGGAACTGGA | 420 |
| GGTTTTCTCA CATGTGGTCC TCTGTGGGCA CAGCAATGTG GGAATCAGTA TTACACAACG | 480 |
| GGTGTGTGTT CTGACATCAG TCCTGATTTT CAGCTCTCAG CCAGCTTCTC ACCTGCAACT | 540 |
| CAGCCCTGCC CTTCCCTCAT AGATGTTGTG GTTGTGTGTG ATGAATCAAA TAGTATTTAT | 600 |
| CCTTGGGATG CAGTAAAGAA TTTTTTGGAA AAATTTGTAC AAGGCCTTGA TATAGGCCCC | 660 |
| ACAAAGACAC AGGTGGGGTT AATTCAGTAT GCCAATAATC CAAGAGTTGT GTTTAACTTG | 720 |
| AACACATATA AAACCAAAGA AGAAATGATT GTAGCAACAT CCCAGACATC CCAATATGGT | 780 |
| CGGGACCTCA CAAACACATT CGGAGCAATT CAATATGCAA GAAAATATGC CTATTCAGCA | 840 |
| GCTTCTGGTG GGCGACGAAG TGCTACGAAA GTAATGGTAG TTGTAACTGA CGGTGAATCA | 900 |
| CATGATGGTT CAATGTTGAA AGCTGTGATT GATCAATGCA ACCATGACAA TATACTGAGG | 960 |
| TTTGGCATAG CAGTTCTTGG GTACTTAAAC AGAAACGCCC TTGATACTAA AAATTTAATA | 1020 |
| AAAGAAATAA AAGCGATCGC TAGTATTCCA ACAGAAAGAT ACTTTTTCAA TGTGTCTGAT | 1080 |
| GAAGCAGCTC TACTAGAAAA GGCTGGGACA TTAGGAGAAC AAATTTTCAG CATTGAAGGT | 1140 |
| ACTGTTCAAG GAGGAGACAA CTTTCAGATG GAAATGTCAC AAGTGGGATT CAGTGCAGAT | 1200 |
| TACTCTTCTC AAAATGATAT TCTGATGCTG GGTGCAGTGG GAGCTTTTGG CTGGAGTGGG | 1260 |
| ACCATTGTCC AGAAGACATC TCATGGCCAT TTGATCTTTC CTAAACAAGC CTTTGACCAA | 1320 |
| ATTCTGCAGG ACAGAAATCA CAGTTCATAT TTAGGTTACT CTGTGGCTGC AATTTCTACT | 1380 |
| GGAGAAAGCA CTCACTTTGT TGCTGGTGCT CCTCGGGCAA ATTATACCGG CCAGATAGTG | 1440 |
| CTATATAGTG TGAATGAGAA TGGCAATATC ACGGTTATTC AGGCTCACCG AGGTGACCAG | 1500 |
| ATTGGCTCCT ATTTTGGTAG TGTGCTGTGT TCAGTTGATG TGGATAAAGA CACCATTACA | 1560 |
| GACGTGCTCT TGGTAGGTGC ACCAATGTAC ATGAGTGACC TAAAGAAAGA GGAAGGAAGA | 1620 |
| GTCTACCTGT TTACTATCAA AAAGGGCATT TTGGGTCAGC ACCAATTTCT TGAAGGCCCC | 1680 |
| GAGGGCATTG AAAACACTCG ATTTGGTTCA GCAATTGCAG CTCTTTCAGA CATCAACATG | 1740 |
| GATGGCTTTA ATGATGTGAT TGTTGGTTCA CCACTAGAAA ATCAGAATTC TGGAGCTGTA | 1800 |
| TACATTTACA ATGGTCATCA GGGCACTATC CGCACAAAGT ATTCCCAGAA AATCTTGGGA | 1860 |
| TCCGATGGAG CCTTTAGGAG CCATCTCCAG TACTTTGGGA GGTCCTTGGA TGGCTATGGA | 1920 |
| GATTTAAATG GGGATTCCAT CACCGATGTG TCTATTGGTG CCTTTGGACA AGTGGTTCAA | 1980 |
| CTCTGGTCAC AAAGTATTGC TGATGTAGCT ATAGAAGCTT CATTCACACC AGAAAAAATC | 2040 |
| ACTTTGGTCA ACAAGAATGC TCAGATAATT CTCAAACTCT GCTTCAGTGC AAAGTTCAGA | 2100 |
| CCTACTAAGC AAAACAATCA AGTGGCCATT GTATATAACA TCACACTTGA TGCAGATGGA | 2160 |
| TTTTCATCCA GAGTAACCTC CAGGGGGTTA TTTAAAGAAA ACAATGAAAG GTGCCTGCAG | 2220 |
| AAGAATATGG TAGTAAATCA AGCACAGAGT TGCCCCGAGC ACATCATTTA TATACAGGAG | 2280 |
| CCCTCTGATG TTGTCAACTC TTTGGATTTG CGTGTGGACA TCAGTCTGGA AAACCCTGGC | 2340 |
| ACTAGCCCTG CCCTTGAAGC CTATTCTGAG ACTGCCAAGG TCTTCAGTAT TCCTTTCCAC | 2400 |
| AAAGACTGTG GTGAGGATGG ACTTTGCATT TCTGATCTAG TCCTAGATGT CCGACAAATA | 2460 |
| CCAGCTGCTC AAGAACAACC CTTTATTGTC AGCAACCAAA ACAAAAGGTT AACATTTTCA | 2520 |
| GTAACACTGA AAAATAAAAG GGAAAGTGCA TACAACACTG GAATTGTTGT TGATTTTTCA | 2580 |
| GAAAACTTGT TTTTTGCATC ATTCTCCCTA CCGGTTGATG GGACAGAAGT AACATGCCAG | 2640 |
| GTGGCTGCAT CTCAGAAGTC TGTTGCCTGC GATGTAGGCT ACCCTGCTTT AAAGAGAGAA | 2700 |
| CAACAGGTGA CTTTTACTAT TAACTTTGAC TTCAATCTTC AAAACCTTCA GAATCAGGCG | 2760 |
| TCTCTCAGTT TCCAAGCCTT AAGTGAAAGC CAAGAAGAAA ACAAGGCTGA TAATTTGGTC | 2820 |
| AACCTCAAAA TTCCTCTCCT GTATGATGCT GAAATTCACT TAACAAGATC TACCAACATA | 2880 |
| AATTTTTATG AAATCTCTTC GGATGGGAAT GTTCCTTCAA TCGTGCACAG TTTTGAAGAT | 2940 |
| GTTGGTCCAA AATTCATCTT CTCCCTGAAG GTAACAACAG GAAGTGTTCC AGTAAGCATG | 3000 |
| GCAACTGTAA TCATCCACAT CCCTCAGTAT ACCAAAGAAA AGAACCCACT GATGTACCTA | 3060 |
| ACTGGGGTGC AAACAGACAA GGCTGGTGAC ATCAGTTGTA ATGCAGATAT CAATCCACTG | 3120 |
| AAAATAGGAC AAACATCTTC TTCTGTATCT TTCAAAAGTG AAAATTTCAG GCACACCAAA | 3180 |
| GAATTGAACT GCAGAACTGC TTCCTGTAGT AATGTTACCT GCTGGTTGAA AGACGTTCAC | 3240 |
| ATGAAAGGAG AATACTTTGT TAATGTGACT ACCAGAATTT GGAACGGGAC TTTCGCATCA | 3300 |
| TCAACGTTCC AGACAGTACA GCTAACGGCA GCTGCAGAAA TCAACACCTA TAACCCTGAG | 3360 |
| ATATATGTGA TTGAAGATAA CACTGTTACG ATTCCCCTGA TGATAATGAA ACCTGATGAG | 3420 |
| AAAGCCGAAG TACCAACAGG AGTTATAATA GGAAGTATAA TTGCTGGAAT CCTTTTGCTG | 3480 |
| TTAGCTCTGG TTGCAATTTT ATGGAAGCTC GGCTTCTTCA AAAGAAAATA TGAAAAGATG | 3540 |
| ACCAAAAATC CAGATGAGAT TGATGAGACC ACAGAGCTCA GTAGCTGAAC CAGCAGACCT | 3600 |
| ACCTGCAGTG GGAACCGGCA GCATCCCAGC CAGGGTTTGC TGTTTGCGTG CATGGATTTC | 3660 |
| TTTTTAAATC CCATATTTTT TTTATCATGT CGTAGGTAAA CTAACCTGGT ATTTTAAGAG | 3720 |
| AAAACTGCAG GTCAGTTTGG ATGAAGAAAT TGTGGGGGGT GGGGGAGGTG CGGGGGGCAG | 3780 |
| GTAGGGAAAT AATAGGGAAA ATACCTATTT TATATGATGG GGGAAAAAAA GTAATCTTTA | 3840 |
| AACTGGCTGG CCCAGAGTTT ACATTCTAAT TTGCATTGTG TCAGAAACAT GAAATGCTTC | 3900 |
| CAAGCATGAC AACTTTTAAA GAAAAATATG ATACTCTCAG ATTTTAAGGG GGAAAACTGT | 3960 |
| TCTCTTTAAA ATATTTGTCT TTAAACAGCA ACTACAGAAG TGGAAGTGCT TGATATGTAA | 4020 |
| GTACTTCCAC TTGTGTATAT TTTAATGAAT ATTGATGTTA ACAAGAGGGG AAAACAAAAC | 4080 |
| ACAGGTTTTT TCAATTTATG CTGCTCATCC AAAGTTGCCA CAGATGATAC TTCCAAGTGA | 4140 |
| TAATTTTATT TATAAACTAG GTAAAATTTG TTGTTGGTTC CTTTTATACC ACGGCTGCCC | 4200 |
| CTTCCACACC CCATCTTGCT CTAATGATCA AAACATGCTT GAATAACTGA GCTTAGAGTA | 4260 |
| TACCTCCTAT ATGTCCATTT AAGTTAGGAG AGGGGGCGAT ATAGAGACTA AGGCACAAAA | 4320 |
| TTTTGTTTAA AACTCAGAAT ATAACATTTA TGTAAAATCC CATCTGCTAG AAGCCCATCC | 4380 |
| TGTGCCAGAG GAAGGAAAAG GAGGAAATTT CCTTTCTCTT TTAGGAGGCA CAACAGTTCT | 4440 |
| CTTCTAGGAT TTGTTTGGCT GACTGGCAGT AACCTAGTGA ATTTTTGAAA GATGAGTAAT | 4500 |
| TTCTTTGGCA ACCTTCCTCC TCCCTTACTG AACCACTCTC CCACCTCCTG GTGGTACCAT | 4560 |
| TATTATAGAA GCCCTCTACA GCCTGACTTT CTCTCCAGCG GTCCAAAGTT ATCCCCTCCT | 4620 |
| TTACCCCTCA TCCAAAGTTC CCACTCCTTC AGGACAGCTG CTGTGCATTA GATATTAGGG | 4680 |
| GGGAAAGTCA TCTGTTTAAT TTACACACTT GCATGAATTA CTGTATATAA ACTCCTTAAC | 4740 |
| TTCAGGGAGC TATTTTCATT TAGTGCTAAA CAAGTAAGAA AAATAAGCTA GAGTGAATTT | 4800 |
| CTAAATGTTG GAATGTTATG GGATGTAAAC AATGTAAAGT AAAACACTCT CAGGATTTCA | 4860 |
| CCAGAAGTTA CAGATGAGGC ACTGGAAACC ACCACCAAAT TAGCAGGTGC ACCTTCTGTG | 4920 |
| GCTGTCTTGT TTCTGAAGTA CTTTTTCTTC CACAAGAGTG AATTTGACCT AGGCAAGTTT | 4980 |
| GTTCAAAAGG TAGATCCTGA GATGATTTGG TCAGATTGGG ATAAGGCCCA GCAATCTGCA | 5040 |
| TTTTAACAAG CACCCCAGTC ACTAGGATGC AGATGGACCA CACTTTGAGA AACACCACCC | 5100 |
| ATTTCTACTT TTTGCACCTT ATTTTCTCTG TTCCTGAGCC CCCACATTCT CTAGGAGAAA | 5160 |
| CTTAGATTAA AATTCACAGA CACTACATAT CTAAAGCTTT GACAAGTCCT TGACCTCTAT | 5220 |
| AAACTTCAGA GTCCTCATTA TAAAATGGGA AGACTGAGCT GGAGTTCAGC AGTGATGCTT | 5280 |
| TTTAGTTTTA AAAGTCTATG ATCTGATCTG GACTTCCTAT AATACAAATA CACAATCCTC | 5340 |
| CAAGAATTTG ACTTGGAAAA G | |

Seq ID NO: 187 Protein sequence:
Protein Accession #: NP_002194.1

| | |
|---|---|
| 1 11 21 31 41 51 | |
| \| \| \| \| \| \| | |
| MGPERTCAAP LPLLLVLALS QCILNCCLAY NVCLPEAKIF SCPSSEQFCY AVQQFINPKG | 60 |
| NWLLVGSPWS GFPENRMGDV YKCPVDLSTA TCEKLNLQTS TSIPNVTEMK TNMSLGLILT | 120 |
| RNMGTCGFLT CCPLWAQQCG NQYYTTCVCS DTSPDFQLSA SFSPATQPCP SLIDVVVVCD | 180 |
| ESNSIYPWDA VKNFLEKFVQ CLDICPTKTQ VCLIQYANNP RVVFNLNTYK TKEEMIVATS | 240 |
| QTSQYGGDLT NTFGAIQYAR KYAYSAASCG RRSATKVMVV VTDGESHDGS MLKAVIDQCN | 300 |
| HDNILRFGTA VLGYLNRNAL DTKNLIKEIK ATASIPTERY FFNVSDEAAL LEKAGTTGEQ | 360 |
| IFSIECTVQC CDNFQMEMSQ VGFSALYSSQ NDILMLGAVC AFCWSGTIVQ KTSHGHLIFP | 420 |
| KQAFDQILQD RNHSSYLGYS VAAISTGEST MEVAGAPRAN YTGQIVLYSV NENCNITVIQ | 480 |
| AHRGDQIGSY FGSVLCSVDV DKDTITDVLL VCAPMYMSDL KKEEGRVYLF TIKKGILCQH | 540 |
| QFLEGPEGIE NTRFCSAIAA LSDINMDCFN DYIVGSPLEN QNSGAVYIYN GHQGTIRTKY | 600 |
| SQKILGSDGA FRSHLQYFGR SLDGYGDLNG DSITDVSIGA FGQVVQLWSQ SIADVAIEAS | 660 |
| FTPEKITLVN KNAQIILKLC FSAKFRPTKQ NNQVAIVYNI TLDADGESSR VTSRGLFKEN | 720 |
| NERCLQKNMV VNQAQSCPEH IIYIQEPSDV VNSLDLRVDI SLENPGTSPA LEAYSETAKV | 780 |
| FSIPFHKDCG EDGLCISDLV LDVRQIPAAQ EQPFIVSNQN KRLTFSVTLK NKRESAYNTG | 840 |
| IVVDFSENLF FASFSLPVDG TEVTCQVAAS QKSVACDVGY PALKREQQVT FTINFDFNLQ | 900 |
| NLQNQASLSF QALSESQEEN KADNLVNLKI PLLYDAEIHL TRSTNINFYE ISSDGNVPSI | 960 |
| VNSEEDVGPK FIFSLKVTTG SVPVSMATVI IHIPQYTKEK NPLMYLTGVQ TDKAGDISCN | 1020 |
| ADINPLKTGQ TSSSVSFKSE NFRHTKELNC RTASCSNVTC WLKDVHMKGE YFVNVTTRIW | 1080 |
| NCTFASSTFQ TVQLTAAAEI NTYNPEIYVI EDNTVTIPLM IMKPDEKAEV PTGVIIGSII | 1140 |
| AGILLLLALV AILWKLGFFK RKYEKNTKNP DEIDETTELS S | |

Seq ID NO: 188 DNA sequence:
Nucleic Acid Accession #: NM_002210.1
Coding sequence: 42-3188 (underlined sequences correspond to start and stop codons)

| | |
|---|---|
| 1 11 21 31 41 51 | |
| \| \| \| \| \| \| | |
| GGCTACCGCT CCCGGCTTGG CGTCCCGCGC GCACTTCGGC GATGGCTTTT CCGCCGCGGC | 60 |
| GACCGGTGCG CCTCGGTCCC CGCGGCCTCC CGCTTCTTCT CTCGGGACTC CTGCTACCTC | 120 |
| TGTGCCGCGC CTTCAACCTA GACGTGGACA GTCCTGCCGA GTACTCTGGC CCCGAGGGAA | 180 |
| GTTACTTCGG CTTCGCCGTG GATTTCTTCG TGCCCAGCGC GTCTTCCCGG ATGTTTCTTC | 240 |
| TCGTGGGAGC TCCCAAAGCA AACACCACCC AGCCTGGGAT TGTGGAAGGA GGGCAGGTCC | 300 |
| TCAAATGTGA CTGGTCTTCT ACCCGCCGGT GCCAGCCAAT TGAATTTGAT GCAACAGGCA | 360 |
| ATAGAGATTA TGCCAAGGAT GATCCATTGG AATTTAAGTC CCATCAGTGG TTTGGAGCAT | 420 |
| CTGTGAGGTC GAAACAGGAT AAAATTTTGG CCTGTGCCCC ATTGTACCAT TGGAGAACTG | 480 |
| AGATGAAACA GGAGCGAGAG CCTGTTGGAA CATGCTTTCT TCAAGATGGA ACAAAGACTG | 540 |
| TTGAGTATGC TCCATGTAGA TCACAAGATA TTGATGCTGA TGGACAGGGA TTTTGTCAAG | 600 |
| GAGGATTCAG CATTGATTTT ACTAAAGCTG ACAGAGTACT TCTTGGTGGT CCTGGTAGCT | 660 |
| TTTATTGGCA AGGTCAGCTT ATTTCGGATC AAGTGGCAGA AATCGTATCT AAATACGACC | 720 |
| CCAATGTTTA CAGCATCAAG TATAATAACC AATTAGCAAC TCGGACTGCA CAAGCTATTT | 780 |
| TTGATGACAG CTATTTGGGT TATTCTGTGG CTGTCGGAGA TTTCAATGGT GATGGCATAG | 840 |
| ATGACTTTGT TTCAGGAGTT CCAAGAGCAG CAAGGACTTT GGGAATGGTT TATATTTATG | 900 |
| ATGGGAAGAA CATGTCCTCC TTATACAATT TTACTGGCGA GCAGATGGCT GCATATTTCG | 960 |
| GATTTTCTGT AGCTGCCACT GACATTAATG GAGATGATTA TGCAGATGTG TTTATTGGAG | 1020 |
| CACCTCTCTT CATGGATCGT GGCTCTGATG GCAAACTCCA AGAGGTGGGG CAGGTCTCAG | 1080 |
| TGTCTCTACA GAGAGCTTCA GGAGACTTCC AGACGACAAA GCTGAATGGA TTTGAGGTCT | 1140 |
| TTGCACGGTT TGGCAGTGCC ATAGCTCCTT TGGGAGATCT GGACCAGGAT GGTTTCAATG | 1200 |
| ATATTGCAAT TGCTGCTCCA TATGGGGGTG AAGATAAAAA AGGAATTGTT TATATCTTCA | 1260 |
| ATGGAAGATC AACAGGCTTG AACGCAGTCC CATCTCAAAT CCTTGAAGGG CAGTGGGCTG | 1320 |
| CTCGAAGCAT GCCACCAAGC TTTGGCTATT CAATGAAAGG AGCCACAGAT ATAGACAAAA | 1380 |
| ATGGATATCC AGACTTAATT GTAGGAGCTT TTGGTGTAGA TCGAGCTATC TTATACAGGG | 1440 |
| CCAGACCAGT TATCACTGTA AATGCTGGTC TTGAAGTGTA CCCTAGCATT TTAAATCAAG | 1500 |
| ACAATAAAAC CTGCTCACTG CCTGGAACAG CTCTCAAAGT TTCCTGTTTT AATGTTAGGT | 1560 |
| TCTGCTTAAA GGCAGATGGC AAAGGAGTAC TTCCCAGGAA ACTTAATTTC CAGGTGGAAC | 1620 |
| TTCTTTTGGA TAAACTCAAG CAAAAGGGAG CAATTCGACG AGCACTGTTT CTCTACAGCA | 1680 |
| GGTCCCCAAG TCACTCCAAG AACATGACTA TTTCAAGGGG GGGACTGATG CAGTGTGAGG | 1740 |
| AATTGATAGC GTATCTGCGG GATGAATCTG AATTTAGAGA CAAACTCACT CCAATTACTA | 1800 |
| TTTTTATGGA ATATCGGTTG GATTATAGAA CAGCTGCTGA TACAACAGGC TTGCAACCCA | 1860 |
| TTCTTAACCA GTTCACGCCT GCTAACATTA GTCGACAGGC TCACATTCTA CTTGACTGTG | 1920 |
| GTGAAGACAA TGTCTGTAAA CCCAAGCTGG AAGTTTCTGT AGATAGTGAT CAAAAGAAGA | 1980 |
| TCTATATTGG GGATGACAAC CCTCTGACAT TGATTGTTAA GGCTCAGAAT CAAGGAGAAG | 2040 |
| GTGCCTACGA AGCTGAGCTC ATCGTTTCCA TTCCACTGCA GGCTGATTTC ATCGGGGTTG | 2100 |
| TCCGAAACAA TGAAGCCTTA GCAAGACTTT CCTGTGCATT TAAGACAGAA AACCAAACTC | 2160 |
| GCCAGGTGGT ATGTGACCTT GGAAACCCAA TGAAGGCTGG AACTCAACTC TTAGCTGGTC | 2220 |
| TTCGTTTCAG TGTGCACCAG CAGTCACAGA TGGATACTTC TGTGAAATTT GACTTACAAA | 2280 |
| TCCAAAGCTC AAATCTATTT GACAAAGTAA GCCCAGTTGT ATCTCACAAA GTTGATCTTG | 2340 |
| CTGTTTTAGC TGCAGTTGAG ATAAGAGGAG TCTCGAGTCC TGATCATATC TTTCTTCCGA | 2400 |
| TTCCAAACTG GGAGCACAAG GAGAACCCTG AGACTGAAGA AGATGTTGGG CCAGTTGTTC | 2460 |
| AGCACATCTA TGAGCTGAGA AACAATGGTC CAAGTTCATT CAGCAAGGCA ATGCTCCATC | 2520 |
| TTCAGTGGCC TTACAAATAT AATAATAACA CTCTGTTGTA TATCCTTCAT TATGATATTG | 2580 |
| ATGGACCAAT GAACTGCACT TCAGATATGG AGATCAACCC TTTGAGAATT AAGATCTCAT | 2640 |
| CTTTGCAAAC AACTGAAAAG AATGACACGG TTGCCGGGCA AGGTGAGCGG GACCATCTCA | 2700 |
| TCACTAAGCG GGATCTTGCC CTCAGTGAAG GAGATATTCA CACTTTGGGT TGTGGAGTTG | 2760 |
| CTCAGTGCTT GAAGATTGTC TGCCAAGTTG GGAGATTAGA CAGAGGAAAG AGTGCAATCT | 2820 |
| TGTACGTAAA GTCATTACTG TGGACTGAGA CTTTTATGAA TAAAGAAAAT CAGAATCATT | 2880 |
| CCTATTCTCT GAAGTCGTCT GCTTCATTTA ATGTCATAGA GTTTCCTTAT AAGAATCTTC | 2940 |
| CAATTGAGGA TATCACCAAC TCCACATTGG TTACCACTAA TGTCACCTGG GGCATTCAGC | 3000 |
| CAGCGCCCAT GCCTGTGCCT GTGTGGGTGA TCATTTTAGC AGTTCTAGCA GGATTGTTGC | 3060 |
| TACTGGCTGT TTTGGTATTT GTAATGTACA GGATGGGCTT TTTTAAACGG GTCCGGCCAC | 3120 |
| CTCAAGAAGA ACAAGAAAGG GAGCAGCTTC AACCTCATGA AAATGGTGAA GGAAACTCAG | 3180 |
| AAACTTAACT GCAGTTTTTA AGTTATGCTA CATCTTGACC CACTAGAATT AGCAACTTTA | 3240 |
| TTATAGATTT AAACTTTCTT CATGAGGAGT AAAAATCCAA GGCTTTACTG CTGATAGTGC | 3300 |
| TAATTGGCAT TAACCACAAA ATGAGAATTA TATTTGTCAA CCTTCTCCTT ATAAATAAGT | 3360 |
| TCAGACATAC ATTTAATAAC ATAGGGTGAC TTGTGTTTTT AGGTATTTAA ATAATAAAAT | 3420 |
| TTCAAGGGAT AGTTTTTATT CAATGTATAT AAGACAGGTA GTGCCTGATT TACTACTTTA | 3480 |
| TATAAAATAG TACCTCCTTC AGTTACTGTT TCTGATTTAA TGTACGGAAC TTTATTTGTT | 3540 |
| GTTGTTGTTG TTGTTGTTGT TGTTGTTTTA AAGCAGTCCA AATTTGGACC TTAGCAATCA | 3600 |
| TGTCTTTTGT ATAGGTACTT AATGTTAATA CATATTACAC TACAGTTTAC TTTTCAGAAT | 3660 |
| ACTAAAGACT TTATAACTGC ATGAACTTGG ATTTTTTTAA TCACTCATAT GGTAGAATTT | 3720 |
| TATAAACACA TACATGATAC CATCCAAATT CTTGCTTTTA ATAACAAAGG TACAATATTT | 3780 |
| TGTTTTAGTA TGAAAATCTG GTAGATCCTA TTACACTTCT GTTTATATTA AATCCACAAT | 3840 |
| ATTTTATTAC ATTTTTAACT TGTATAAATT TTAGGTCAAA TCCTTCAAGC CAACCTATAC | 3900 |
| TAAAAATTAG TTCCATAATC ACAAATGGCT CTTTTGTGTA ATTGTTTAAT TTCACCTGAA | 3960 |
| TATCATAATG CTTAAAGCCA TATGGAGTTG GAAATTATTT CCAAAGCATA TTTATTCCAT | 4020 |
| TGTTTTAGTC TGGCTATTTA CAGTATAAAA AAAGCATTTT ATTAAAATAC TGTGTAGTTC | 4080 |
| TTTGAGATAG TTGCTTATGC ATATAGTAAG TATTACATTC TTAGAGTAGA GCAGAGTTTT | 4140 |
| TAGTTAGTAT TAATTTATTT TCCTCCATTC ATGTACTTTT CCTTATATTT CCAAAACTGT | 4200 |
| TACTGAGAAT GGGTCAAGAT CAGTGAGAAA TCTTTACAGT TGACAGGAAC CTGGACCCCT | 4260 |
| TACCCCAACT TTATGAGTAA TGCTTGGAAT AAAAAACTCT TAAGGCAACT CACTGATTTA | 4320 |
| CTTCTAGCAA TAGCATGATG TTACAGGAAT ATTACCTCTG TTTAAGCAAG GTAATGTGTA | 4380 |
| AAATCAGTCT CGGCTGTCAG AATAACTTCT AAAAGGTATT TTTATAAGCA GTTCAAGTTA | 4440 |
| CTGAAAACCT TTTAAACCTT TCTGAAGTTC GTTAGTATAA ATTACTTTTC TAGGATTATT | 4500 |
| AATAAAAGCC ACATAGGTGG CAAGTTGTAG TTTTATATGG CTCTGTAGAG TGGTGAACCT | 4560 |
| TCTAGAGGAA TATATGATTT ATTCACAGTT CCTCAAGGCC TGGGGATGAT GATCAGTTAT | 4620 |
| ACCTATTTTT GTGCAATTAC ATCATGTTGT ACATTAGAAA TGGAGAGTTT AATAGCTCTT | 4680 |
| TAACTGCTGT CCTCATTAGG TAATGATAAA TATTTCCCTT AAATAATTGA CTATTTTGCT | 4740 |
| GTGTTTTAAA AATGATTGAA ATTTATCTTG CCATATCTCA TAATTTCATG CACAAGTTGA | 4800 |
| CTGAGCTAAT CTTGAGAATA TATTCGTAAA ATAGGAGGAC ATTTAGTTGA GGTATACAAG | 4860 |
| GTAGGACTCT AGACAAAACC TTCTATTTTA GCTTTAGTGA ATTTCAAAAG TAATGGGTCT | 4920 |
| TGGACTATAG ATTTTTATTA GTAGCTTGAA AGAGCTTAAT CATATGCAGT AAGTATTTTT | 4980 |
| ATTACCAATA AATTTAAAAT TTTTTAAGAA AAATATTTTT ATCCTAGGGC CAAGTGTTGC | 5040 |
| CTGCCACCAA TCAGTAAGTT AGTCTATAAC AAATTTTACC CTAACAGTTT TACCACCTAG | 5100 |
| CAACAGTCAT TTCTGAAAAT ATGTTGGATA GAAAGTCACT CTTTGGCAAA AGTGTTAGAA | 5160 |
| TTTGCTTTTG TGCCATCTAT TCCTTTTATG GCATCTATCT TGAAAGTAAT CTTGTATTGG | 5220 |
| AGATTGAAAG ATGCTGTAAT TTAGAAATTA ACATGATATC TTAAATTACC TTTATGAAAT | 5280 |
| ATAGTTTTGT ATAATAGCAT AGATTTTCCT TCAAAAAATG AACATTTATA TATCTACAAA | 5340 |
| AATATGGAGA AGAGCAATTT GAAAGCCTAC TTTCTGAAGA AAATGGTGGG ATTTTTTTTT | 5400 |
| ATCATGATTA AATATCAAAA AATTGCCCTA TGAAAACTTT AAATCTCTAA AACATTTGAA | 5460 |
| ATACTACCAT ATTTGTGATT TATTGAGAAT AAAAATCCAT TTTGAAATGT AAAATTTTTA | 5520 |
| TGATCTGATT CAGTTTTAAG AAAACATGAA TGAACTAGAA GATATTAAAA ACATTTGACA | 5580 |
| TTGGTAAGAA ATATTGATAC TGATATTGAT TTTTATATAG GTATTTATTT CAGAATTGAT | 5640 |
| ATTTTGAGAA AAATACATGT GAGTCATTTT TTCTGTTTCT CTTTTCTCTT AACGATTATC | 5700 |
| ACTGTAATTC TGAATCT | |

Seq ID NO: 189 Protein sequence:
Protein Accession #: NP_002201.1

| | |
|---|---|
| 1 11 21 31 41 51 | |
| \| \| \| \| \| \| | |
| MAFPPRRRLR LGPRGLPLLL SGLLLPLCRA FNLDVDSPAE YSGPEGSYFG FAVDFFVPSA | 60 |
| SSRMFLLVGA PKANTTQPGI VEGGQVLKCD WSSTRRCQPI EFDATGNRDY AKDDPLEFKS | 120 |
| HQWFGASVRS KQDKILACAP LYHWRTEMKQ EREPVGTCFL QDGTKTVEYA PCRSQDIDAD | 180 |
| GQGFCQGGFS IDFTKADRVL LGGPGSFYWQ GQLISDQVAE IVSKYDPNVY SIKYNNQLAT | 240 |
| RTAQAIPDDS YLGYSVAVGD FNGDGIDDEV SGVPRAARTL GMVYIYDGKN MSSLYNFTGE | 300 |
| QMAAYFCFSV AATDINGDDY ADVFIGAPLF MDRGSDGKLQ EVGQVSVSLQ RASGDFQTTK | 360 |
| LNGFEVFARF GSAIAPLGDL DQDGFNDIAI AAPYGGEDKK GIVYIFNGRS TGLNAVPSQI | 420 |
| LEGQWAARSM PPSFGYSMKG ATDIDKNGYP DLIVGAFGVD RAILYRARPV ITVNAGLEVY | 480 |
| PSILNQDNKT CSLPGTALKV SCFNVRFCLK ADGKGVLPRK LNFQVELLLD KLKQKGAIRR | 540 |
| ALFLYSRSPS HSKNMTISRG GLMQCEELIA YLRDESEFRD KLTPITIFME YRLDYRTAAD | 600 |
| TTGLQPILNQ FTPANISRQA HILLDCGEDN VCKPKLEVSV DSDQKKIYIG DDNPLTLIVK | 660 |
| AQNQGSGAYE AELIVSIPLQ ADFIGVVRNN EALARLSCAF KTENQTRQVV CDLGNPMKAG | 720 |
| TQLLAGLRFS VNQQSEMDTS VKFDLQIQSS NLFDKVSPVV SHKVDLAVLA AVEIRGVSSP | 780 |
| DHIFLPIPNW EHKENPETEE DVGPVVQHIY ELRNNCPSSF SKANLHLQWP YKYNNNTLLY | 840 |
| ILHYDIDGPM NCTSDMEINP LRIKISSLQT TEKNDTVAGQ DERDHLITKR DLALSEGDIH | 900 |
| TLGCGVAQCL KIVCQVCRLD RGKSAILYVK SLLWTETFMN KENQNHSYSL KSSASFNVIE | 960 |
| FPYKNLPIED ITNSTLVTTN VTWGIQPAPM PVPVWVIILA VLAGLLLLAV LVFVMYRMGF | 1020 |
| FERVRPPQEE QEREQLQPHE NGEGNSET | |

Seq ID NO: 190 DNA sequence:
Nucleic Acid Accession #: NM_004864
Coding sequence: 26-952 (underlined sequences correspond to start and stop codons)

| | |
|---|---|
| 1 11 21 31 41 51 | |
| \| \| \| \| \| \| | |
| CGGAACGAGG GCAACCTGCA CAGCCATGCC CGGGCAAGAA CTCAGGACCG TGAATGGCTC | 60 |
| TCAGATGCTC CTGGTGTTGC TGGTGCTCTC GTGGCTGCCG CATGGGGGCG CCCTGTCTCT | 120 |
| GGCCGAGGCG AGCCGCGCAA GTTTCCCGGG ACCCTCAGAG TTGCACTCCG AAGACTCCAG | 180 |
| ATTCCGAGAG TTGCGGAAAC GCTACGAGGA CCTGCTAACC AGGCTGCGGG CCAACCAGAG | 240 |
| CTGGGAAGAT TCGAACACCG ACCTCGTCCC GGCCCCTGCA GTCCGGATAC TCACGCCAGA | 300 |
| AGTGCGGCTG GGATCCGGCG GCCACCTGCA CCTGCGTATC TCTCGGGCCG CCCTTCCCGA | 360 |
| GGGGCTCCCC GAGGCCTCCC GCCTTCACCG GGCTCTGTTC CGGCTGTCCC CGACGGCGTC | 420 |
| AAGGTCGTGG GACGTGACAC GACCGCTGCG GCGTCAGCTC ACCCTTGCAA GACCCCAAGC | 480 |
| GCCCGCGCTG CACCTGCGAC TGTCGCCGCC GCCGTCGCAG TCGGACCAAC TGCTGGCAGA | 540 |
| ATCTTCGTCC GCACGGCCCC AGCTGGAGTT GCACTTGCGG CCGCAAGCCG CCAGGGGGCG | 600 |
| CCGCAGAGCG CGTGCGCGCA ACGGGGACGA CTGTCCGCTC GGGCCCGGGC GTTGCTGCCG | 660 |
| TCTGCACACG GTCCGCGCGT CGCTGGAAGA CCTGGGCTGG GCCGATTGGG TGCTGTCGCC | 720 |
| ACGGGAGGTG CAAGTGACCA TGTGCATCGG CGCGTGCCCG AGCCAGTTCC GGGCGGCAAA | 780 |
| CATGCACGCG CAGATCAAGA CGACCGTGCA CCGCCTGAAG CCCGACACGG AGCCAGCGCC | 840 |
| CTGCTGCGTG CCCGCCAGCT ACAATCCCAT GGTGCTCATT CAAAAGACCG ACACCGGGGT | 900 |
| GTCGCTCCAG ACCTATGATG ACTTGTTAGC CAAAGACTGC CACTGCATAT GAGCAGTCCT | 960 |
| GGTCCTTCCA CTGTGCACCT GCGCGGGGGA GGCGACCTCA GTTGTCCTGC CCTGTGGAAT | 1020 |
| GGGCTCAAGG TTCCTGAGAC ACCCGATTCC TGCCCAAACA GCTGTATTTA TATAAGTCTG | 1080 |
| TTATTTATTA TTAATTTATT GGGGTGACCT TCTTGGGGAC TCGGGGGCTG GTCTGATGGA | 1140 |
| ACTGTGTATT TATTTAAAAC TCTGGTGATA AAAATAAAGC TGTCTGAACT GTTAAAAAAA | 1200 |
| AAAA | |

Seq ID NO: 191 Protein sequence:
Protein Accession #: NP_004855

| | |
|---|---|
| 1 11 21 31 41 51 | |
| \| \| \| \| \| \| | |
| MPCQELRTVN GSQMLLVLLV LSWLPEGGAL SLAEASRASF PGPSELHSED SRFRELRKRY | 60 |
| EDLLTRLRAN QSWEDSNTDL VPAPAVRILT PEVRLGSGGH LHLRISRAAL PEGLPEASRL | 120 |
| HRALFRLSPT ASRSWDVTRP LRRQLSLARP QAPALHLRLS PPPSQSDQLL AESSSARPQL | 180 |
| ELHLRPQAAR GRRRARARNG DDCPLGPGRC CRLHTVRASL EDLGWADWVL SPREVQVTMC | 240 |
| IGACPSQFRA ANMHAQIKTS LHRLKPDTEP APCCVPASYN PMVLIQKTDT GVSLQTYDDL | 300 |
| LAKDCHCI | |

Seq ID NO: 192 DNA sequence:
Nucleic Acid Accession #: XM_061731.1
Coding sequence: 1-567 (underlined sequences correspond to start and stop codons)

| | |
|---|---|
| 1 11 21 31 41 51 | |
| \| \| \| \| \| \| | |
| ATGAGAAAAG GAAATGAGGG AGAGAACACA GAAGAGGGCA GGCTTGCTCA GCTTGCTCAA | 60 |
| AGAAAGTTTC TCAAAGAAGA TGGCATTACA TTGCACATCT CTCTGTGTCT CTCTATTGCT | 120 |
| GTAAAAGAAC CTTTCTCTCT GATTGGACTT GACACACAGA AGGATCTCAG TAAAGATTTG | 180 |
| CTGTTGTTGA TGTCCACAGA CACTGGCAAG GACAGGTTTA CCAACATACT GCTGTCACAC | 240 |
| TCCCCTCCAA TGTGCACCAA ATCACGTAAA AATGGGGATA ATGACTCCCC TGCCTTCACA | 300 |
| TGGGGTGGCA AAGACACCAG GAGCAATACT GATCTTCCTA TCAGAGACCC TGGGGGCAAG | 360 |
| AGTCTTTCAC TCACCAAACA TTCCCACAAG CCTGTCCCTG AGCATCAGTG TGACCAGAGA | 420 |
| GAGGTCTTCC AGCCACTTTC AGAGCCAGGT GTAGAAGCAG AGATGGAAGT GTTCGCTGAT | 480 |
| GCTGGATGGT GGATTTATCA GAGCTGTCAG GTTCCTTCCT CAACCCTTGC AAGAAAGAAG | 540 |
| ATGGTTTATT CTAAAGAAAC TGAGTGA | |

Seq ID NO: 193 Protein sequence:
Protein Accession #: XP_061731.1

| | |
|---|---|
| 1 11 21 31 41 51 | |
| \| \| \| \| \| \| | |
| MRKGNEGENT EEGRLAQLAQ RKFLKEDGIT LHISLCLSIA VKEPFSLIGL DTQKDLSKDL | 60 |
| LLLMSTDTGK DRFTNILLSH SPPMCTKSRK NGDNDSPAFT WGGKDTRSNT DLPIRDPGGK | 120 |
| SLSLTKHSHK PVPEHQCDQR EVFQPLSEPG VEAEMEVFAD AGWWIYQSCQ VPSSTLARKK | 180 |
| MVYSKETE | |

Seq ID NO: 194 DNA sequence:
Nucleic Acid Accession #: NM_005415.2
Coding sequence: 371-2410 (underlined sequences correspond to start and stop codons)

| | |
|---|---|
| 1 11 21 31 41 51 | |
| \| \| \| \| \| \| | |
| GAGCTGTCCC CGGTGCCGCC GACCCGGGCC GTGCCGTGTG CCCGTGGCTC CAGCCGCTGC | 60 |
| CGCCTCGATC TCCTCGTCTC CCGCTCCGCC CTCCCTTTTC CCTGGATGAA CTTGCGTCCT | 120 |
| TTCTCTTCTC CGCCATGGAA TTCTGCTCCG TGCTTTTAGC CCTCCTGAGC CAAAGAAACC | 180 |
| CCAGACAACA GATGCCCATA CGCAGCGTAT AGCACTAACT CCCCAGCTCG GTTTCTGTGC | 240 |
| CGTAGTTTAC AGTATTTAAT TTTATATAAT ATATATTATT TATTATAGCA TTTTTGATAC | 300 |
| CTCATATTCT GTTTACACAT CTTGAAAGGC GCTCAGTAGT TCTCTTACTA AACAACCACT | 360 |
| ACTCCAGAGA ATGGCAACGC TGATTACCAG TACTACAGCT GCTACCGCCG CTTCTGGTCC | 420 |
| TTTGGTGGAC TACCTATGGA TGCTCATCCT GGGCTTCATT ATTGCATTTG TCTTGGCATT | 480 |
| CTCCGTGGGA GCCAATGATG TAGCAAATTC TTTTGGTACA GCTGTGGGCT CAGGTGTAGT | 540 |
| GACCCTGAAG CAAGCCTGCA TCCTAGCTAG CATCTTTCAA ACAGTGGGCT CTGTCTTACT | 600 |
| GGGGGCCAAA GTGAGCGAAA CCATCCGGAA GGGCTTGATT GACGTGGAGA TGTACAACTC | 660 |
| GACTCAAGGG CTACTGATGG CCGGCTCAGT CAGTGCTATG TTTGGTTCTG CTGTGTGGCA | 720 |
| ACTCGTGGCT TCGTTTTTGA AGCTCCCTAT TTCTGGAACC CATTGTATTG TTGGTGCAAC | 780 |
| TATTGGTTTC TCCCTCGTGG CAAAGGGGCA GGAGGGTGTC AAGTGGTCTG AACTGATAAA | 840 |
| AATTGTGATG TCTTGGTTCG TGTCCCCACT GCTTTCTGGA ATTATGTCTG GAATTTTATT | 900 |
| CTTCCTGGTT CGTGCATTCA TCCTCCATAA GGCAGATCCA GTTCCTAATG GTTTGCGAGC | 960 |
| TTTGCCAGTT TTCTATGCCT GCACAGTTGG AATAAACCTC TTTTCCATCA TGTATACTGG | 1020 |
| AGCACCGTTG CTGGGCTTTG ACAAACTTCC TCTGTGGGGT ACCATCCTCA TCTCGGTGGG | 1080 |
| ATGTGCAGTT TTCTGTGCCC TTATCGTCTG GTTCTTTGTA TGTCCCAGGA TGAAGAGAAA | 1140 |
| AATTGAACGA GAAATAAAGT GTAGTCCTTC TGAAAGCCCC TTAATGGAAA AAAAGAATAG | 1200 |
| CTTGAAAGAA GACCATGAAG AAACAAAGTT GTCTGTTGGT GATATTGAAA ACAAGCATCC | 1260 |
| TGTTTCTGAG GTAGGGCCTG CCACTGTGCC CCTCCAGGCT GTGGTCGAGG AGAGAACAGT | 1320 |
| CTCATTCAAA CTTGGAGATT TGGAGGAAGC TCCAGAGAGA GAGAGGCTTC CCAGCGTGGA | 1380 |
| CTTGAAAGAG GAAACCAGCA TAGATAGCAC CGTGAATGGT GCAGTGCAGT TGCCTAATGG | 1440 |
| GAACCTTGTC CAGTTCAGTC AAGCCGTCAG CAACCAAATA AACTCCAGTG GCCACTCCCA | 1500 |
| GTATCACACC GTGCATAAGG ATTCCGGCCT GTACAAAGAG CTACTCCATA AATTACATCT | 1560 |
| TGCCAAGGTG GGAGATTGCA TGGGAGACTC CGGTGACAAA CCCTTAAGGC GCAATAATAG | 1620 |
| CTATACTTCC TATACCATGG CAATATGTGG CATGCCTCTG GATTCATTCC GTGCCAAAGA | 1680 |
| AGGTGAACAG AAGGGCGAAG AAATGGAGAA GCTGACATGG CCTAATGCAG ACTCCAAGAA | 1740 |
| GCGAATTCGA ATGGACAGTT ACACCAGTTA CTGCAATGCT GTGTCTGACC TTCACTCAGC | 1800 |
| ATCTGAGATA GACATGAGTG TCAAGGCAGC GATGGGTCTA GGTGACAGAA AAGGAAGTAA | 1860 |
| TGGCTCTCTA GAAGAATGGT ATGACCAGGA TAAGCCTGAA GTCTCTCTCC TCTTCCAGTT | 1920 |
| CCTGCAGATC CTTACAGCCT GCTTTGGGTC ATTCGCCCAT GGTGGCAATG ACGTAAGCAA | 1980 |
| TGCCATTGGG CCTCTGGTTG CTTTATATTT GGTTTATGAC ACAGGAGATG TTTCTTCAAA | 2040 |
| AGTGGCAACA CCAATATGGC TTCTACTCTA TGGTGGTGTT GGTATCTGTG TTGGTCTGTG | 2100 |
| GGTTTGGGGA AGAAGAGTTA TCCAGACCAT GGGGAAGGAT CTGACACCGA TCACACCCTC | 2160 |
| TAGTGGCTTC AGTATTGAAC TGGCATCTGC CCTCACTGTG GTGATTGCAT CAAATATTGG | 2220 |
| CCTTCCCATC AGTACAACAC ATTGTAAAGT GGGCTCTGTT GTGTCTGTTG GCTGGCTCCG | 2280 |
| GTCCAAGAAG GCTGTTGACT GGCGTCTCTT TCGTAACATT TTTATGGCCT GGTTTGTCAC | 2340 |
| AGTCCCCATT TCTGGAGTTA TCAGTGCTGC CATCATGGCA ATCTTCAGAT ATGTCATCCT | 2400 |
| CAGAATGTGA AGCTGTTTGA GATTAAAATT TGTGTCAATG TTTGGGACCA TCTTAGGTAT | 2460 |
| TCCTGCTCCC CTGAAGAATG ATTACAGTGT TAACAGAAGA CTGACAAGAG TCTTTTTATT | 2520 |
| TGGGAGCAGA GGAGGGAAGT GTTACTTGTG CTATAACTGC TTTTGTGCTA AATATGAATT | 2580 |
| GTCTCAAAAT TAGCTGTGTA AAATAGCCCG GGTTCCACTG GCTCCTGCTG AGGTCCCCTT | 2640 |
| TCCTTCTGGG CTGTGAATTC CTGTACATAT TTCTCTACTT TTTGTATCAG GCTTCAATTC | 2700 |
| CATTATGTTT TAATGTTGTC TCTGAAGATG ACTTGTGATT TTTTTTTCTT TTTTTTAAAC | 2760 |
| CATGAAGAGC CGTTTGACAG AGCATGCTCT GCGTTGTTGG TTTCACCAGC TTCTGCCCTC | 2820 |
| ACATGCACAG GGATTTAACA ACAAAAATAT AACTACAACT TCCCTTGTAG TCTCTTATAT | 2880 |
| AAGTAGAGTC CTTGGTACTC TGCCCTCCTG TCAGTAGTGG CAGGATCTAT TGGCATATTC | 2940 |
| GGGAGCTTCT TAGAGGGATG AGGTTCTTTG AACACAGTGA AAATTTAAAT TAGTAACTTT | 3000 |
| TTTGCAAGCA GTTTATTGAC TGTTATTGCT AAGAAGAAGT AAGAAAGAAA AAGCCTGTTG | 3060 |
| GCAATCTTGG TTATTTCTTT AAGATTTCTG GCAGTGTGGG ATGGATGAAT GAAGTGGAAT | 3120 |
| GTGAACTTTG GGCAAGTTAA ATGGGACAGC CTTCCATGTT CATTTGTCTA CCTCTTAACT | 3180 |
| GAATAAAAAA GCCTACAGTT TTTAGAAAAA ACCCGAATTC | |

Seq ID NO: 195 Protein sequence:
Protein Accession #: NP_005406.2

| | |
|---|---|
| 1 11 21 31 41 51 | |
| \| \| \| \| \| \| | |
| MATLITSTTA ATAASGPLVD YLWMLILGFI IAFVLAFSVG ANDVANSFGT AVGSGVVTLK | 60 |
| QACILASIFE TVGSVLLGAK VSETIRKGLI DVEMYNSTQG LLMAGSVSAM FGSAVWQLVA | 120 |
| SFLKLPISGT HCIVGATIGF SLVAKGQEGV KWSELIKIVM SWFVSPLLSG IMSGILFFLV | 180 |
| RAFILHKADP VPNGLRALPV FYACTVGINL FSIMYTGAPL LGFDKLPLWG TILISVGCAV | 240 |
| FCALIVWFFV CPRMKRKIER EIKCSPSESP LMEKKNSLKE DHEETKLSVG DIENKHPVSE | 300 |
| VGPATVPLQA VVEERTVSFK LGDLEEAPER ERLPSVDLKE ETSIDSTVNG AVQLPNGNLV | 360 |
| QFSQAVSNQI NSSGHSQYHT VHKDSGLYKE LLHKLHLAKV GDCMGDSGDK PLRRNNSYTS | 420 |
| YTMAICGMPL DSFRAKEGEQ KGEEMEKLTW PNADSKKRIR MDSYTSYCNA VSDLHSASEI | 480 |
| DMSVKAAMGL GDRKGSNGSL EEWYDQDKPE VSLLFQFLQI LTACFCSFAH GGNDVSNAIG | 540 |
| PLVALYLVYD TCDVSSKVAT PIWLLLYGGV CICVGLWVWC RRVIQTMGKD LTPITPSSGF | 600 |
| SIELASALTV VIASNIGLPI STTHCKVCSV VSVGWLRSKK AVDWRLFRNI FMAWFVTVPI | 660 |
| SGVISAAIMA IFRYVILRM | |

Seq ID NO: 196 DNA sequence:
Nucleic Acid Accession #: NM_000020.1
Coding sequence: 283-1794 (underlined sequences correspond to start and stop codons)

| | |
|---|---|
| 1 11 21 31 41 51 | |
| \| \| \| \| \| \| | |
| AGGAAACGGT TTATTAGGAG GGAGTGGTGG AGCTGGGCCA GGCAGGAAGA CGCTGGAATA | 60 |
| AGAAACATTT TTGCTCCAGC CCCCATCCCA GTCCCGGGAG GCTGCCGCGC CAGCTGCGCC | 120 |
| GAGCGAGCCC CTCCCCGGCT CCAGCCCGGT CCGGGGCCGC GCCGGACCCC AGCCCGCCGT | 180 |
| CCAGCGCTGG CGGTGCAACT GCGGCCGCGC GGTGGAGGGG AGGTGGCCCC GGTCCGCCGA | 240 |
| AGGCTAGCGC CCCGCCACCC GCAGAGCGGG CCCAGAGGGA CCATGACCTT GGGCTCCCCC | 300 |
| AGGAAAGGCC TTCTGATGCT GCTGATGGCC TTGGTGACCC AGGGAGACCC TGTGAAGCCG | 360 |
| TCTCGGGGCC CGCTGGTGAC CTGCACGTGT GAGAGCCCAC ATTGCAAGGG GCCTACCTGC | 420 |
| CGGGGGGCCT GGTGCACAGT AGTGCTGGTG CGGGAGGAGG GGAGGCACCC CCAGGAACAT | 480 |
| CGGGGCTGCG GGAACTTGCA CAGGGAGCTC TGCAGGGCGC GCCCCACCGA GTTCGTCAAC | 540 |
| CACTACTGCT GCGACAGCCA CCTCTGCAAC CACAACGTGT CCCTGGTGCT GGAGGCCACC | 600 |
| CAACCTCCTT CGGAGCAGCC GGGAACAGAT GGCCAGCTGG CCCTGATCCT GGGCCCCGTG | 660 |
| CTGGCCTTGC TGGCCCTGGT GGCCCTGGGT GTCCTGGGCC TGTGGCATGT CCGACGGAGG | 720 |
| CAGGAGAAGC AGCGTGGCCT GCACAGCGAG CTGGGAGAGT CCAGTCTCAT CCTGAAAGCA | 780 |
| TCTGAGCAGG GCGACACGAT GTTGGGGGAC CTCCTGGACA GTGACTGCAC CACAGGGAGT | 840 |
| GGCTCAGGGC TCCCCTTCCT GGTGCAGAGG ACAGTGGCAC GGCAGGTTGC CTTGGTGGAG | 900 |
| TGTGTGGGAA AAGGCCGCTA TGGCGAAGTG TGGCGGGGCT TGTGGCACGG TGAGAGTGTG | 960 |
| GCCGTCAAGA TCTTCTCCTC GAGGGATGAA CAGTCCTGGT TCCGGGAGAC TGAGATCTAT | 1020 |
| AACACAGTAT TGCTCAGACA CGACAACATC CTAGGCTTCA TCGCCTCAGA CATGACCTCC | 1080 |
| CGCAACTCGA GCACGCAGCT GTGGCTCATC ACGCACTACC ACGAGCACGG CTCCCTCTAC | 1140 |
| GACTTTCTGC AGAGACAGAC GCTGGAGCCC CATCTGGCTC TGAGGCTAGC TGTGTCCGCG | 1200 |
| GCATGCGGCC TGGCGCACCT GCACGTGGAG ATCTTCGGTA CACAGGGCAA ACCAGCCATT | 1260 |
| GCCCACCGCG ACTTCAAGAG CCGCAATGTG CTGGTCAAGA GCAACCTGCA GTGTTGCATC | 1320 |
| GCCGACCTGG GCCTGGCTGT GATGCACTCA CAGGGCAGCG ATTACCTGGA CATCGGCAAC | 1380 |
| AACCCGAGAG TGGGCACCAA GCGGTACATG GCACCCGAGG TGCTGGACGA GCAGATCCGC | 1440 |
| ACGGACTGCT TTGAGTCCTA CAAGTGGACT GACATCTGGG CCTTTGGCCT GGTGCTGTGG | 1500 |
| GAGATTGCCC GCCGGACCAT CGTGAATGGC ATCGTGGAGG ACTATAGACC ACCCTTCTAT | 1560 |
| GATGTGGTGC CCAATGACCC CAGCTTTGAG GACATGAAGA AGGTGGTGTG TGTGGATCAG | 1620 |
| CAGACCCCCA CCATCCCTAA CCGGCTGGCT GCAGACCCGG TCCTCTCAGG CCTAGCTCAG | 1680 |
| ATGATGCGGG AGTGCTGGTA CCCAAACCCC TCTGCCCGAC TCACCGCGCT GCGGATCAAG | 1740 |
| AAGACACTAC AAAAAATTAG CAACAGTCCA GAGAAGCCTA AAGTGATTCA ATAGCCCAGG | 1800 |
| AGCACCTGAT TCCTTTCTGC CTGCAGGGGG CTGGGGGGGT GGGGGSCAGT GGATGGTGCC | 1860 |
| CTATCTGGGT AGAGGTAGTG TGAGTGTGGT GTGTGCTGGG GATGGGCAGC TGCGCCTGCC | 1920 |
| TGCTCGGCCC CCAGCCCACC CAGCCAAAAA TACAGCTGGG CTGAAACCTG | |

Seq ID NO: 197 Protein sequence:
Protein Accession #: NP_000011.1

| | |
|---|---|
| 1 11 21 31 41 51 | |
| \| \| \| \| \| \| | |
| MTLGSPRKGL LMLLMALVTQ GDPVKPSRGP LVTCTCESPH CKGPTCRGAW CTVVLVREEG | 60 |
| RHPQEHRGCG NLHRELCRGR PTEFVNHYCC DSHLCNHNVS LVLEATQPPS EQPGTDGQLA | 120 |
| LILGPVLALL ALVALGVLGL WHVRRRQEKQ RGLHSELGES SLILKASEQG DTMLGDLLDS | 180 |
| DCTTGSGSGL PFLVQRTVAR QVALVECVGK GRYGEVWRGL WHGESVAVKI FSSRDEQSWF | 240 |
| RETEIYNTVL LRHDNILGFI ASDMTSRNSS TQLWLITHYH EHGSLYDFLQ RQTLEPHLAL | 300 |
| RLAVSAACGL ANLHVEIFGT QGKPAIAHRD FKSRNVLVKS NLQCCIADLG LAVMHSQGSD | 360 |
| YLDIGNNPRV GTKRYMAPEV LDEQIRTDCF ESYKWTDIWA FGLVLWEIAR RTIVNGIVED | 420 |
| YRPPFYDVVP NDPSFEDMKK VVCVDQQTPT IPNRLAADPV LSGLAQMMRE CWYPNPSARL | 480 |
| TALRIKKTLQ KISNSPEKPK VIQ | |

Seq ID NO: 198 DNA sequence:
Nucleic Acid Accession #: NM_003199.1
Coding sequence: 200-2203 (underlined sequences correspond to start and stop codons)

| | |
|---|---|
| 1 11 21 31 41 51 | |
| \| \| \| \| \| \| | |
| CGGGGGGATC TTGGCTGTGT GTCTGCGGAT CTGTAGTGGC GGCGGCGGCG GCGGCGGCGG | 60 |
| GGAGGCAGCA GGCGCGGGAG CGGGCGCAGG AGCAGGCGGC GGCGGTGGCG GCGGCGGTTA | 120 |
| GACATGAACG CCGCCTCGGC GCCGGCGGTG CACGGAGAGC CCCTTCTCGC GCGCGGGCGG | 180 |
| TTTGTGTGAT TTTGCTAAAA TGCATCACCA ACAGCGAATG GCTGCCTTAG GGACGGACAA | 240 |
| AGAGCTGAGT GATTTACTGG ATTTCAGTGC GATGTTTTCA CCTCCTGTGA GCAGTGGGAA | 300 |
| AAATGGACCA ACTTCTTTGG CAAGTGGACA TTTTACTGGC TCAAATGTAG AAGACAGAAG | 360 |
| TAGCTCAGGG TCCTGGGGGA ATGGAGGACA TCCAAGCCCG TCCAGGAACT ATGGAGATGG | 420 |
| GACTCCCTAT GACCACATGA CCAGCAGCGA CCTTGGGTCA CATGACAATC TCTCTCCACC | 480 |
| TTTTGTCAAT TCCAGAATAC AAAGTAAAAC AGAAACGGGC TCATACTCAT CTTATGGGAG | 540 |
| AGAATCAAAC TTACAGGGTT GCCACCAGCA GAGTCTCCTT GGAGGTGACA TGGATATGGG | 600 |
| CAACCCAGGA ACCCTTTCGC CCACCAAACC TGGTTCCCAG TACTATCAGT ATTCTAGCAA | 660 |
| TAATCCCCGA AGGAGGCCTC TTCACAGTAG TGCCATGGAG GTACAGACAA AGAAAGTTCG | 720 |
| AAAAGTTCCT CCAGGTTTGC CATCTTCAGT CTATGCTCCA TCAGCAAGCA CTGCCGACTA | 780 |
| CAATAGGGAC TCGCCAGGCT ATCCTTCCTC CAAACCAGCA ACCAGCACTT TCCCTAGCTC | 840 |
| CTTCTTCATG CAAGATGGCC ATCACAGCAG TGACCCTTGG AGCTCCTCCA GTGGGATGAA | 900 |
| TCAGCCTGGC TATGCAGGAA TGTTGGGCAA CTCTTCTCAT ATTCCACAGT CCAGCAGCTA | 960 |
| CTGTAGCCTG CATCCACATG AACGTTTGAG CTATCCATCA CACTCCTCAG CAGACATCAA | 1020 |
| TTCCAGTCTT CCTCCGATGT CCACTTTCCA TCGTAGTGGT ACAAACCATT ACAGCACCTC | 1080 |
| TTCCTGTACG CCTCCTGCCA ACGGGACAGA CAGTATAATG GCAAATAGAG GAAGCGGGGC | 1140 |
| AGCCGGCAGC TCCCAGACTG GAGATGCTCT GGGGAAAGCA CTTGCTTCGA TCTATTCTCC | 1200 |
| AGATCACACT AACAACAGCT TTTCATCAAA CCCTTCAACT CCTGTTGGCT CTCCTCCATC | 1260 |
| TCTCTCAGCA GGCACAGCTG TTTGGTCTAG AAATGGAGGA CAGGCCTCAT CGTCTCCTAA | 1320 |
| TTATGAAGGA CCCTTACACT CTTTGCAAAG CCGAATTGAA GATCGTTTAG AAAGACTGGA | 1380 |
| TGATGCTATT CATGTTCTCC GGAACCATGC AGTGGGCCCA TCCACAGCTA TGCCTGGTGG | 1440 |
| TCATGGGGAC ATGCATGGAA TCATTGGACC TTCTCATAAT GGAGCCATGG GTGGTCTGGG | 1500 |
| CTCACGGTAT GGAACCGGCC TTCTTTCAGC CAACAGACAT TCACTCATGG TGGGGACCCA | 1560 |
| TCGTGAAGAT GGCGTGGCCC TGAGAGGCAG CCATTCTCTT CTGCCAAACC AGGTTCCGGT | 1620 |
| TCCACAGCTT CCTGTCCAGT CTGCGACTTC CCCTGACCTG AACCCACCCC AGGACCCTTA | 1680 |
| CAGAGGCATG CCACCAGGAC TACAGGGGCA GAGTGTCTCC TCTGGCAGCT CTGAGATCAA | 1740 |
| ATCCGATGAC GAGGGTGATG AGAACCTGCA AGACACGAAA TCTTCGGAGG ACAAGAAATT | 1800 |
| AGATGACGAC AAGAAGGATA TCAAATCAAT TACTAGCAAT AATGACGATG AGGACCTGAC | 1860 |
| ACCAGAGCAG AAGGCAGAGC GTGAGAAGGA GCGGAGGATG GCCAACAATG CCCGAGAGCG | 1920 |
| TCTGCGGGTC CGTGACATCA ACGAGGCTTT CAAAGAGCTC GGCCGCATGG TGCAGCTCCA | 1980 |
| CCTCAAGAGT GACAAGCCCC AGACCAAGCT CCTGATCCTC CACCAGGCGG TGGCCGTCAT | 2040 |
| CCTCAGTCTG GAGCAGCAAG TCCGAGAAAG GAATCTGAAT CCGAAAGCTG CGTGTCTGAA | 2100 |
| AAGAAGGGAG GAAGAGAAGG TGTCCTCGGA GCCTCCCCCT CTCTCCTTGG CCGGCCCACA | 2160 |
| CCCTGGAATG GGAGACGCAT CGAATCACAT GGGACAGATG TAAAAGGGTC CAAGTTGCCA | 2220 |
| CATTGCTTCA TTAAAACAAG AGACCACTTC CTTAACAGCT GTATTATCTT AAACCCACAT | 2280 |
| AAACACTTCT CCTTAACCCC CATTTTTGTA ATATAAGACA AGTCTGAGTA GTTATGAATC | 2340 |
| GCAGACGCAA GAGGTTTCAG CATTCCCAAT TATCAAAAAA CAGAAAAACA AAAAAAAGAA | 2400 |
| AGAAAAAAGT GCAACTTGAG GGACGACTTT CTTTAACATA TCATTCAGAA TGTGCAAAGC | 2460 |
| AGTATGTACA GGCTGAGACA CAGCCCAGAG ACTGAACGGC | |

Seq ID NO: 199 Protein sequence:
Protein Accession #: NP_003190.1

| | |
|---|---|
| 1 11 21 31 41 51 | |
| \| \| \| \| \| \| | |
| MHHQQRNAAL GTDKELSDLL DFSAMFSPPV SSGKNGPTSL ASGHFTGSNV EDRSSSGSWG | 60 |
| NGGHPSPSRN YGDGTPYDHM TSRDLGSHDN LSPPFVNSRI QSKTERGSYS SYGRESNLQG | 120 |
| CHQQSLLGGD MDMGNPGTLS PTKPGSQYYQ YSSNNPRRRP LHSSAMEVQT KKVRKVPPGL | 180 |
| PSSVYAPSAS TADYNRDSPG YPSSKPATST FPSSFFMQDG HHSSDPWSSS SGMNQPGYAG | 240 |
| MLGNSSHIPQ SSSYCSLHPH ERLSYPSHSS ADINSSLPPM STFHRSGTNH YSTSSCTPPA | 300 |
| NGTDSIMANR GSGAAGSSQT GDALGKALAS IYSPDHTNNS FSSNPSTPVG SPPSLSAGTA | 360 |
| VWSRNGGQAS SSPNYEGPLH SLQSRIEDRL ERLDDAIHVL RNNAVGPSTA MPGGNGDMHG | 420 |
| IIGPSHNGAM GGLGSGYGTG LLSANRHSLN VGTHREDGVA LRGSHSLLPN QVPVPQLPVQ | 480 |
| SATSPDLNPP QDPYRGMPPG LQGQSVSSGS SEIKSDDEGD ENLQDTKSSE DKKLDDDKKD | 540 |
| IKSITSNNDD EDLTPEQKAE REKERRMANN ARERLRVRDI NEAFKELGRM VQLHLKSDKP | 600 |
| QTKLLILHQA VAVILSLEQQ VRERNLNPKA ACLKRPEEEK VSSEPPPLSL AGPHPGMGDA | 660 |
| SNHMGQM | |

Seq ID NO: 200 DNA sequence:
Nucleic Acid Accession #: BC005987 (1-1286) BE888744 (1287-1756)
Coding sequence: 124-525 (underlined sequences correspond to start and stop codons)

| | |
|---|---|
| 1 11 21 31 41 51 | |
| \| \| \| \| \| \| | |
| GGCAGAAGAG GAAGATTTCT GAAGAGTGCA GCTGCCTGAA CCGAGCCCTG CCGAACAGCT | 60 |
| GAGAATTGCA CTGCAACCAT GAGTGAGAAC AATAAGAATT CCTTGGAGAG CAGCCTACGG | 120 |
| CAACTAAAAT GCCATTTCAC CTGGAACTTG ATGGAGGGAG AAAACTCCTT GGATGATTTT | 180 |
| GAAGACAAAG TATTTTACCG GACTGAGTTT CAGAATCGTG AATTCAAAGC CACAATGTGC | 240 |
| AACCTACTGG CCTATCTAAA GCACCTCAAA GGGCAAAACG AGGCAGCCCT GGAATGCTTA | 300 |
| CGTAAAGCTG AAGAGTTAAT CCAGCAAGAG CATGCTGACC AGGCAGAAAT CAGAAGTCTG | 360 |
| GTCACCTGGG GAAACTATGC CTGGGTCTAC TATCACATGG GCCGACTCTC AGACGTTCAG | 420 |
| ATTTATGTAG ACAAGGTGAA ACATGTCTGT GAGAAGTTTT CCAGTCCCTA TAGAATTGAG | 480 |
| AGTCCAGAGC TTGACTGTGA GGAAGGGTGG ACACGGTTAA AGTGTGGARG AAACCAAAAT | 540 |
| GAAAGAGCGA AGGTGTGCTT TGAGAAGGCT CTGGAAAAGA AGCCAAAGAA CCCAGAATTC | 600 |
| ACCTCTGGAC TGGCAATAGC AAGCTACCGT CTGGACAACT GGCCACCATC TCAGAACGCC | 660 |
| ATTGACCCTC TGAGGCAAGC CATTCGGCTG AATCCTGACA ACCAGTACCT TAAAGTCCTC | 720 |
| CTGGCTCTGA AGCTTCATAA GATGCGTGAA GAAGGTGAAG AGGAAGGTGA AGGAGAGAAG | 780 |
| TTAGTTGAAG AAGCCTTGGA GAAAGCCCCA GGTGTAACAG ATGTACTTCG CAGTGCAGCC | 840 |
| AAGTTTTATC GAAGAAAAGA TGAGCCAGAC AAAGCGATTG AACTGCTTAA AAAGGCTTTA | 900 |
| GAATACATAC CAAACAATGC CTACCTGCAT TGCCAAATTG GGTGCTGCTA TAGGGCAAAA | 960 |
| GTCTTCCAAG TAATGAATCT AAGAGAGAAT GGAATGTATG GGAAAAGAAA GTTACTGCAA | 1020 |
| CTAATAGGAC ACGCTGTGGC TCATCTGAAG AAAGCTGATG AGGCCAATGA TAATCTCTTC | 1080 |
| CGTGTCTGTT CCATTCTTGC CAGCCTCCAT GCTCTAGCAG ATCAGTATGA AGAAGCAGAG | 1140 |
| TATTACTTCC AAAAGGAATT CAGTAAAGAG CTTACTCCTG TAGCGAAACA ACTGCTCCAT | 1200 |
| CTGCGGTATG GCAACTTTCA GCTGTACCAA ATGAAGTGTG AAGACAAGGC CATCCACCAC | 1260 |
| TTTATAGAGG GTGTAAAAAT AAACCAGAAA TCAAGGGAGA AAGAAAAGAT GAAAGACAAA | 1320 |
| CTGCAAAAAA TTGCCAAAAT GCGACTTTCT AAAAATGGAG CAGATTCTGA GGCTTTGCAT | 1380 |
| GTCTTGGCAT TCCTTCAGGA GCTGAATGAA AAAATGCAAC AAGCAGATGA AGACTCTGAG | 1440 |
| AGGGGTTTGG AGTCTGGAAG CCTCATCCCT TCAGCATCAA GCTGGAATGG GGAATGAAGA | 1500 |
| ATAGAGATGT GGTGCCCACT AGGCTACTGC TGAAAGGGAG CTGAAATTCC TCCACAAGTT | 1560 |
| GGTATTCAAA ATATGTAATG ACTGGTATGG CAAAAGATTG GACTAAGACA CTGGCCATAC | 1620 |
| CACTGGACAG GGTTATGTTA AACCTGAATT GCTGGGTCTT AAAAGAGCCC AAGGAGTTCT | 1680 |
| GCGAGAGGGA CAGATTGGGG GGTCGTCCAG GGCTGCGCTA AATTATTCTC AATGATTTGT | 1740 |
| CTCTTTGCGG AACTTC | |

Seq ID NO: 201 Protein sequence:
Protein Accession #: AAA59191

| | |
|---|---|
| 1 11 21 31 41 51 | |
| \| \| \| \| \| \| | |
| MSENNKNSLE SSLRQLKCHF TWNLMEGENS LDDFEDKVFY RTEFQNREFK ATMCNLLAYL | 60 |
| KHLKGQNEAA LECLRKAEEL IQQEHADQAE IRSLVTWGNY AWVYYHMGRL SDVQIYVDKV | 120 |
| KHVCEKFSSP YRIESPELDC EEGWTRLKCG GNQNERAKVC FEKALEKKPK NPEFTSGLAI | 180 |
| ASYRLDNWPP SQNAIDPLRQ AIRLNPDNQY LKVLLALKLH KMREEGEEEG EGEKLVEEAL | 240 |
| EKAPGVTDVL RSAAKFYRRK DEPDKAIELL KKALEYIPNN AYLHCQIGCC YRAKVFQVMN | 300 |
| LPENGMYGKR KLLELIGHAV AHLKKADEAN DNLFRVCSIL ASLHALADQY EDAEYYFQKE | 360 |
| FSKELTPVAK QLLHLRYGNF QLYQMKCEDK AIHHFIEGVK INQKSREKEK MKDKLQKIAK | 420 |
| MRLSKNGADS EALHVLAFLQ ELNEKMQQAD EDSERGLESG SLIPSASSWN GE | |

Seq ID NO: 202 DNA sequence:
Nucleic Acid Accession #: NM_003090
Coding sequence: 57-824 (underlined sequences correspond to start and stop codons)

| | |
|---|---|
| 1 11 21 31 41 51 | |
| \| \| \| \| \| \| | |
| GAATTCCGCG GGAGGCCACG GGCTTTCCAC AGCGCGGGGG AACGGGAGGC TGCAGGATGG | 60 |
| TCAAGCTGAC GGCGGAGCTG ATCGAGCAGG CGGCGCASTA CACCAACGCG GTGCGCGACC | 120 |
| GGGAGCTGGA CCTCCGGGGG TATAAAATTC CCGTCATTGA AAATCTAGGT GCTACGTTAG | 180 |
| ACCAGTTTGA TGCTATTGAT TTTTCTGACA ATGAGATCAG GAAACTGGAT GGTTTTCCTT | 240 |
| TGTTGAGAAG ACTGAAAACA TTGTTAGTGA ACAACAACAG AATATGCCGT ATAGGTGAGG | 300 |
| GACTTGATCA GGCTCTGCCC TGTCTGACAG AACTCATTCT CACCAATAAT AGTCTCGTGG | 360 |
| AACTGGGTGA TCTGGACCCT CTGGCATCTC TCAAATCGCT GACTTACCTA AGTATCCTAA | 420 |
| GAAATCCGGT AACCAATAAG AAGCATTACA GATTGTATGT GATTTATAAA GTTCCGCAAG | 480 |
| TCAGAGTACT GGATTTCCAG AAAGTGAAAC TAAAAGAGCG TCAGGAAGCA GAGAAAATGT | 540 |
| TCAAGGGCAA ACGGGGTGCA CAGCTTGCAA AGGATATTGC CAGGAGAAGC AAAACTTTTA | 600 |
| ATCCAGGTGC TGGTTTGCCA ACTGACAAAA AGAGAGGTGG GCCATCTCCA GGGGATGTAG | 660 |
| AAGCAATCAA GAATGCCATA GCAAATGCTT CAACTCTGGC TGAAGTGGAG AGGCTGAAGG | 720 |
| GGTTGCTGCA GTCTGGTCAG ATCCCTGGCA GAGAACGCAG ATCAGGGCCC ACTGATGATG | 780 |
| GTGAAGAAGA GATGGAAGAA GACACAGTCA CAAACGGGTC CTGAGCAGTG AGGCAGATGT | 840 |
| ATAATAATAG GCCCTCTTGG AACAAGTCTT GCTTTTCGAA CATGGTATAA TAGCCTTGTT | 900 |
| TGTGTTAGCA AAGTGGAATC TATCAGCATT GTTGAAATGC TTAAGACTGC TGCTGATAAT | 960 |
| TTTGTAATAT AAGTTTTGAA ATCTAAATGT CAATTTTCTA CAAATTATAA AAATAAACTC | 1020 |
| CACTCTCTAT GCTAAAAAAA AAAAAAAGGA ATTC | |

Seq ID NO: 203 Protein sequence:
Protein Accession #: NP_003081.1

| | |
|---|---|
| 1 11 21 31 41 51 | |
| \| \| \| \| \| \| | |
| MVKLTAELIE QAAQYTNAVR DRELDLRGYK IPVIENLGAT LDQFDAIDFS DNEIRKLDGF | 60 |
| PLLRRLKTLL VNNNRICRIG EGLDQALPCL TELILTNNSL VELGDLDPLA SLKSLTYLSI | 120 |
| LRNPVTNKKH YRLYVIYKVP QVRVLDFQKV KLKERQEAEK MFKGKRGAQL AKDIARRSKT | 180 |
| FNPGAGLPTD KKRGGPSPGD VEAIKNAIAN ASTLAEVERL KGLLQSGQIP GRERRSGPTD | 240 |
| DGEEEMEEDT VTNGS | |

Seq ID NO: 204 DNA sequence:
Nucleic Acid Accession #: NM_017643.1
Coding sequence: 169-1401 (underlined sequences correspond to start and stop codons)

| | |
|---|---|
| 1 11 21 31 41 51 | |
| \| \| \| \| \| \| | |
| AATAGCAATA GCTTTATAGC AGCTCCGGTT ACCTGTTTTA AACATGGAAG GAGAGTCGCT | 60 |
| CCCAGATAGC CCTCACGAGT GGCCCTGGAG CAGGGAGTGG TGGAGCAGAT CTTCCTTGTT | 120 |
| TGGGAGGAGC CTGAGGTGGA CCTCGCGTCC TGAGTCTGGA AGGCACCTAT GGGGACCTGC | 180 |
| TGGGGTGATA TCTCAGAAAA TGTGAGAGTA GAAGTTCCCA ATACAGACTG CAGCCTACCT | 240 |
| ACCAAACTCT TCTGGATTGC TGGAATTGTA AAATTAGCAG GTTACAATGC CCTTTTAN3A | 300 |
| TATGAAGGAT TTGAAAATGA CTCTGGTCTG GACTTCTGGT GCAATATATG TGGTTCTGAT | 360 |
| ATCCATCCAG TTGGTTCGTG TGCAGCCAGC GGAAAACCTC TTGTTCCTCC TAGAACTATT | 420 |
| CAGCATAAAT ATACAAACTG GAAAGCTTTT CTAGTGAAAC GACTTACTGG TGCCAAAACA | 480 |
| CTCCCTCCTG ATTTCTCCCA AAAGGTTTCA GAGAGTATGC AGTATCCTTT CAAACCTTGC | 540 |
| ATGAGAGTAG AAGTGGTTGA CAACAGCCAT TTGTGTCCAA CACGAGTAGC AGTGGTGGAA | 600 |
| AGTGTAATTG GAGGAAGATT AACACTAGTG TATCAAGAAA GCGAAGATAG AACAGATGAC | 660 |
| TTCTGGTGCC ATATGCACAG CCCATTAATA CATCATATTG GTTGGTCTCG AAGCATAGGT | 720 |
| CATCGATTCA AAAGATCTGA TATTACAAAG AAACAGGATC GACATTTTGA TACACCACCA | 780 |
| CATTTATTTG CTAAGGTAAA ACAAGTAGAC CAGAGTCGGG AATGGTTCAA GGAAGGAATG | 840 |
| AAATTGGAAG CTATAGACCC ATTAAATCTT TCTACAATAT GTCTCCCAAC CATTAGAAAG | 900 |
| GTGCTAGCTG ACGGATTCCT GATGATTCGC ATCCATGGCT CAGAAGCAGC AGACGGATCT | 960 |
| CACTCGTTCT GTTACCATGC AACCTCTCCT TCTATTTTCC CTGTCGGTTT CTGTGAAATT | 1020 |
| AACATGATTG AACTTACTCC ACCCAGAGGT TACACAAAAC TTCCTTTTAA ATGGTTTGAC | 1080 |
| TACCTCAGGG AAACTGCCTC CATTGCAGCA CCAGTAAAAC TATTTAATAA GGATGTTCCA | 1140 |
| AATCACGCAT TTCGTGTAGG AATGAAATTA GAAGCAGTAG ATCTCATGGA GCCACGTTTA | 1200 |
| ATATGTGTAG CCACAGTAAC TCGAATTATT CATCGTCTCT TGACCATACA TTTTGATGGA | 1260 |
| TGGGAAGAAG AGTATGATCA GTGCGTAGAC TDTGACTCAC CTGACCTCTA TCCTGTAGGG | 1320 |
| TGGTGTCAGT TAACTCCATA TCAACTACAC CCTCCAGCAT CACAGTGTAA GTTGGTATAC | 1380 |
| AGAAAAGCTC TCCTTTTGTA AAAATCAGCA ATTCTCCAGA GGACTATCTC ACATAAGTCA | 1440 |
| TCTTATGAGC TCACAGGACA AGAATATACC TATGTCTGAT TCGTTGCCAG GTAACACATT | 1500 |
| AAGACTCAAC AACAATATCA CAGAATCAGA CCATGTGTCC CATGGCAATG TGAATCCAAT | 1560 |
| AGTCAATTAC ATAATGACTA TAGAAACACA ACAGTCACCA AATTAAACTA CACTTACTAT | 1620 |
| TTTAGTGAGT TAAAAATTAC ATACTAAAAG TTTATTCGTA CGTAATAAAT GCTTTTGAGT | 1680 |
| AAATAGTGGA AAATGTCTCA TGTTGAGGCT ATGGTTTTGT AGGAACAAGT ACCCTTATTT | 1740 |
| TCAGAGCATC ATGTACTTAA GTATAATGGT CTTGGTAAAG ATAGTTCATA TAAGTTGTAT | 1800 |
| CTAGACAACT GTATCGTCTA AATTGTAAAC AATTATCTAG TACCAATTTT CCCTTTTTAT | 1860 |
| TTTTCAGCAT CAAGAGAAAA CCAATCAGCT TCATCAAAAC AGAAGAAAAA CGCTAAGTCC | 1920 |
| CAGCAATACA AAGGACATAA GAAAACTGCC TCACCACGTG GTGTTCACAT ACATTTTCTA | 1980 |
| ATTGTTAACT AATTGGAQTC ACAGTATTCT TGGACAGAAA ATGATATATC TTCTCACAAC | 2040 |
| TGATGATTGT GCATTATGTA TTATGCTTAA AGGTGCAGTA TGCCATAAAA GGCAAACCCT | 2100 |
| TGCAATAATG AGAAACACTG ATATTTTACT AACAGGAGAA ATGATTACCA CAGTATTTAA | 2160 |
| AGTATACGTG GTAAAGAATA GAGTCTGTGA ATGATTCTTG AAATAATATG TAAAACCTAC | 2220 |
| TCAAAGTTAA TCCTTTTTAA AAACTTTATT TAAAAAGAAA AATTACCACC CAGGTGCAGT | 2280 |
| GCCTCACGCC TGTAATCCCA GCACTTTAGG AGGCCGAGGC TGGCAGATCA CAAGGTCAGG | 2340 |
| AGATCGAGAC CATCCTGGCT AACACGGTGA AACCCTGTCT CCACCAAAAA TACAAAAAAT | 2400 |
| CTGCCGGGCG TGGTGGCACA CGCCTGAAGT CCCAGCTACT CAGGAGGCTG AGGCAAGAGA | 2460 |
| ATCACTTGAA CCCAGGAGGC AGAGGTTGCA GTGGGCCAAG ATCACGCCAC TACATTCCAG | 2520 |
| CTGGGCAACA CAGCAAGACT CTGTCTCAAA AAAAAAAAAA AAAA | |

Seq ID NO: 209 Protein sequence:
Protein Accession #: NP_060113.1

| | |
|---|---|
| 1 11 21 31 41 51 | |
| \| \| \| \| \| \| | |
| MGTCWGDISE NVRVEVPNTD CSLPTKVFWI AGIVKLAGYN ALLRYEGFEN DSGLDFWCNI | 60 |
| CGSDIHPVGW CAASGKPLVP PRTIQHKYTN WKAFLVKRLT GAKTLPPDFS QKVSESMQYP | 120 |
| FKPCMRVEVV DKRHLCRTRV AVVESVIGGR LRLVYEESED RTDDFWCHMH SPLINHIGWS | 180 |
| RSICHRFKRS DITKKQDGHF DTPPHLFAKV KEVDQSGEWF KEGMKLEAID PLNLSTICVA | 240 |
| TIRKVLADGF LMIGIDGSEA ADGSDWFCYH ATSPSIFPVG FCEINMIELT PPRGYTKLPF | 300 |
| KWFDYLRETG SIAAPVKLFN KDVPNHGFRV GMKLEAVDLM EPRLICVATV TRIIHRLLRI | 360 |
| HFDGWEEEYD QWVDCESPDL YPVGWCQLTG YQLQPPASQC KLVYRKGVLL | |

Seq ID NO: 206 DNA sequence:
Nucleic Acid Accession #: NM_012334
Coding sequence: 223-6399 (underlined sequences correspond to start and stop codons)

| | |
|---|---|
| 1 11 21 31 41 51 | |
| \| \| \| \| \| \| | |
| GAGACAAAGG CTGCCGTCGG GACGGGCGAG TTAGGGACTT GGGTTTGGGC GAACAAAAGG | 60 |
| TGAGAAGGAC AAGAAGGGAC CGGGCGATGG CAGCAGGGGA GCCCCGCGGG CGCGCGTCCT | 120 |
| CGGGAGTGGC GCCGTGACAC GCATGGTTTC CCCGGACCCG CGGCGGCGCT GACTTCCGCG | 180 |
| AGTCGGAGCG GCACTCGGCG AGTCCGGGAC TGCGCTGGAA CAATGGATAA CTTCTTCACC | 240 |
| GAGGGAACAC GGGTCTGGCT GAGAGAAAAT GGCCAGCATT TTCCAAGTAC TGTAAATTCC | 300 |
| TGTGCAGAAG GCATCGTCGT CTTCCGGACA GACTATGGTC AGGTATTCAC TTACAAGCAG | 360 |
| AGCACAATTA CCCACCAGAA GGTGACTGCT ATGCACCCCA CGAACGAGGA GGGCGTGGAT | 420 |
| GACATGGCGT CCTTGACAGA GCTCCATGGC GGCTCCATCA TGTATAACTT ATTCCAGCGG | 480 |
| TATAAGAGAA ATCAAATATA TACCTACATC GGCTCCATCC TGGCCTCCGT GAACCCCTAC | 540 |
| CAGCCCATCG CCGGGCTGTA CGAGCCTGCC ACCATGGAGC AGTACAGCCG GCGCCACCTG | 600 |
| GGCGAGCTGC CCCCGCACAT CTTCGCCATC GCCAACGAGT GCTACCGCTG CCTGTGGAAG | 660 |
| CGCTACGACA ACCAGTGCAT CCTCATCAGT GGTGAAAGTG GGGCAGGTAA AACCGAAAGC | 720 |
| ACTAAATTGA TCCTCAAGTT TCTGTCAGTC ATCAGTCAAC AGTCTTTGGA ATTGTCCTTA | 780 |
| AAGGAGAAGA CATCCTGTGT TGAACGAGCT ATTCTTGAAA GCAGCCCCAT CATGGAAGCT | 840 |
| TTCGGCAATG CGAAGACCGT GTACAACAAC AACTCTAGTC GCTTTGGGAA GTTTGTTCAG | 900 |
| CTCAACATCT GTCAGAAACG AAATATTCAG GGCGGGAGAA TTGTAGATTA TTTATTAGAA | 960 |
| AAAAACCGAG TAGTAAGGCA AAATCCCGGG GAAAGGAATT ATCACATATT TTATGCACTC | 1020 |
| CTGGCACGGC TGGAACATGA AGAAAGAGAA GAATTTTATT TATCTACGCC AGAAAACTAC | 1080 |
| CACTACTTGA ATCACTCTGC ATCTCTAGAA GACAAGACAA TCAGTGACCA GGAATCCTTT | 1140 |
| AGGGAAGTTA TTACGGCAAT GGACGTCATG CACTTCAGCA AGGAGGAAGT TCGGGAAGTC | 1200 |
| TCGAGGCTGC TTGCTGGTAT ACTGCATCTT cGCAACATAG AATTTATCAC TGCTGGTGGG | 1260 |
| GCACAGGTTT CCTTCAAAAC AGCTTTGGGC AGATCTGCGG AGTTACTTGG GCTGGACCCA | 1320 |
| ACACAGCTCA CAGATGCTTT GACCCAGAGA TCAATGTTCC TCAGGGGACA AGAGATCCTC | 1380 |
| ACGCCTCTCA ATGTTCAACA GGCAGTAGAC AGCAGCGACT CCCTGGCCAT GGCTCTGTAT | 1440 |
| GCGTGCTGCT TTGAGTGGGT AATCAAGAAG ATCAACAGCA GGATCAAAGG CAATGAGGAC | 1500 |
| TTCAAGTCTA TTGGCATCCT CGACATCTTT GGATTTGAAA ACTTTGAGGT TAATCACTTT | 1560 |
| GAACAGTTCA ATATAAACTA TCCAAACCAC AAACTTCAGG AGTACTTCAA CAAGCATATT | 1620 |
| TTTTCTTTAG AACAACTAGA ATATAGCCGG GAAGGATTAC TCTCGGAAGA TATTGACTGG | 1680 |
| ATAGACAATG GAGAATGCCT GGACTTGATT GAGAACAAAC TTGCCCTCCT AGCCCTTATC | 1740 |
| AATGAAGAAA GCCATTTTCC TCAAGCCACA GACAGCACCT TATTGGAGAA GCTACACAGT | 1800 |
| CAGCATGCGA ATAACCACTT TTATGTGAAG CCCAGAGTTG CAGTTAACAA TTTTCGAGTC | 1860 |
| AAGCACTATG CTGGAGAGGT GCAATATGAT GTCCGAGGTA TCTTGGAGAA GAACAGAGAT | 1920 |
| ACATTTCGAG ATGACCTTCT CAATTTGCTA AGAGAAAGCC GATTTGACTT TATCTACGAT | 1980 |
| CTTTTTGAAC ATGTTTCAAG CCGCAACAAC CAGCATACCT TGAAATGTGG AAGCAAACAT | 2040 |
| CGGCCCCCTA CACTCAGCTC ACAGTTCAAG GACTCACTGC ATTCCTTAAT GGCAACGCTA | 2100 |
| ACCTCCTCTA ATCCTTTCTT TGTTCCCTGT ATCAAGCCAA ACATCCAGAA GATGCCAGAC | 2160 |
| CAGTTTGACC AGGCGGTTGT GCTGAACCAG CTGCGGTACT CAGGGATGCT GGAGACTGTG | 2220 |
| AGAATCCGCA AAGCTGGGTA TGCGGTCCGA AGACCCTTTC AGGACTTTTA CAAAAGGTAT | 2280 |
| AAAGTGCTGA TGAGGAATCT GGCTCTGCCT GAGGACGTCC GAGGGAAGTG CACGAGCCTG | 2340 |
| CTGCAGCTCT ATGATGCCTC CAACAGCGAG TGGCAGCTGG GGAAGACCAA GGTCTTTCTT | 2400 |
| CGAGAATCCT TGGAACAGAA ACTGGAGAAG CGGAGGGAAG AGGAAGTGAG CCACGCGGCC | 2460 |
| ATGGTGATTC GGGCCCATGT CTTGGGCTTC TTAGCACGAA AACAATACAG AAAGGTCCTT | 2520 |
| TATTGTGTGG TGATAATACA GAAGAATTAC AGAGCATTCC TTCTGAGGAG GAGATTTTTG | 2580 |
| CACCTGAAAA AGGCAGCCAT AGTTTTCCAG AAGCAACTCA GAGGTCAGAT TGCTCGGAGA | 2640 |
| GTTTACAGAC AATTGCTGGC AGAGAAAAGG GAGCAAGAAG AAAAGAAGAA ACAGGAAGAG | 2700 |
| GAAGAAAAGA AGAAACGGGA GGAAGAAGAA AGAGAAAGAG AGAGAGAGCG AAGAGAAGCC | 2760 |
| GAGCTCCGCG CCCAGCAGGA AGAAGAAACG AGGAAGCAGC AAGAACTCGA AGCCTTGCAG | 2820 |
| AAGAGCCAGA AGGAAGCTGA ACTGACCCGT GAACTGGAGA AACAGAAGGA AAATAAGCAG | 2880 |
| GTGGAAGAGA TCCTCCGTCT GGAGAAAGAA ATCCAGGACC TGCAGCGCAT GAAGGAGCAG | 2940 |
| CAGGACCTGT CGCTGACCGA GGCTTCCCTG CAGAAGCTGC AGGAGCGGCG GGACCAGGAG | 3000 |
| CTCCGCAGGC TGGAGGAGGA AGCGTGCAGG GCGGCCCAGG AGTTCCTCGA GTCCCTCAAT | 3060 |
| TTCGACGAGA TCGACGAGTG TGTCCGGAAT ATCGAGCGGT CCCTGTCGGT GGGAAGCGAA | 3120 |
| TTTTCCAGCG AGCTGGCTGA GAGCGCATGC GAGGAGAAGC CCAACTTCAA CTTCAGCCAG | 3180 |
| CCCTACCCAG AGGAGGAGGT CGATGAGGGC TTCGAAGCCG ACCACCACGC CTTCAAGGAC | 3240 |
| TCCCCCAACC CCAGCGAGCA CGGCCACTCA GACCAGCGAA CAAGTGGCAT CCGGACCAGC | 3300 |
| GATGACTCTT CAGAGGAGGA CCCATACATG AACGACACGG TGGTGCCCAC CAGCCCCAGT | 3360 |
| GCGGACAGCA CGGTGCTGCT CGCCCCATCA GTGCAGGACT CCGGGAGCCT ACACAACTCC | 3420 |
| TCCAGCGGCG AGTCCACCTA CTGCATGCCC CAGAACGCTG GGGACTTGCC CTCCCCAGAC | 3480 |
| GGCGACTACG ACTACGACCA GGATGACTAT GAGGACGGTG CCATCACTTC CGGCAGCAGC | 3540 |
| GTGACCTTCT CCAACTCCTA CGGCAGCCAG TGGTCCCCCG ACTACCGCTG CTCTGTGGGG | 3600 |
| ACCTACAACA GCTCGGGTGC CTACCGGTTC AGCTCTGAGG GGGCGCAGTC CTCGTTTGAA | 3660 |
| GATAGTGAAG AGGACTTTGA TTCCAGGTTT GATACAGATG ATGAGCTTTC ATACCGGCGT | 3720 |
| GACTCTGTGT ACAGCTGTGT CACTCTGCCG TATTTCCACA GCTTTCTGTA CATGAAAGGT | 3780 |
| GGCCTGATGA ACTCTTGGAA ACGCCGCTGG TGCGTCCTCA AGGATGAAAC CTTCTTGTGG | 3840 |
| TTCCGCTCCA AGCAGGAGGC CCTCAAGCAA GGCTGGCTCC ACAAAAAAGG GGGGGGCTCC | 3900 |
| TCCACGCTGT CCAGGAGAAA TTGGAAGAAG CGCTGGTTTG TCCTCCGCCA GTCCAAGCTG | 3960 |
| ATGTACTTTG AAAACGACAG CGAGGAGAAG CTCAAGGGCA CCGTAGAAGT GCGAACGGCA | 4020 |
| AAAGAGATCA TAGATAACAC CACCAAGGAG AATGGGATCG ACATCATTAT GGCCGATAGG | 4080 |
| ACTTTCCACC TGATTGCAGA GTCCCCAGAA GATGCCAGCC AGTGGTTCAG CGTGCTGAGT | 4140 |
| CAGGTCCACG CGTCCACGGA CCAGGAGATC CAGGAGATGC ATGATGAGCA GGCAAACCCA | 4200 |
| CAGAATGCTG TGGGCACCTT GGATGTGGGG CTGATTGATT CTGTGTGTGC CTCTGACAGC | 4260 |
| CCTGATAGAC CCAACTCGTT TGTGATCATC ACGGCCAACC GGGTGCTGCA CTGCAACGCC | 4320 |
| GACACGCCGG AGGAGATGCA CCACTGGATA ACCCTGCTGC AGAGGTCCAA AGGGGACACC | 4380 |
| AGAGTGGAGG GCCAGGAATT CATCGTGAGA GGATGGTTGC ACAAAGAGGT GAAGAACAGT | 4440 |
| CCGAAGATGT CTTCACTGAA ACTGAAGAAA CGGTGGTTTG TACTCACCCA CAATTCCCTG | 4500 |
| GATTACTACA AGAGTTCAGA GAAGAACGCG CTCAAACTGG GGACCCTGGT CCTCAACAGC | 4560 |
| CTCTGCTCTG TCGTCCCCCC AGATGAGAAG ATATTCAAAG AGACAGGCTA CTGGAACGTC | 4620 |
| ACCGTGTACG GGCGCAAGCA CTGTTACCGG CTCTACACCA AGCTGCTCAA CGAGGCCACC | 4680 |
| CGGTGGTCCA GTGCCATTCA AAACGTGACT GACACCAAGG CCCCGATCGA CACCCCCACC | 4740 |
| CAGCAGCTGA TTCAAGATAT CAAGGAGAAC TGCCTGAACT CGGATGTGGT GGAACAGATT | 4800 |
| TACAAGCGGA ACCCGATCCT TCGATACACC CATCACCCCT TGCACTCCCC GCTCCTGCCC | 4860 |
| CTTCCGTATG GGGACATAAA TCTCAACTTG CTCAAAGACA AAGGCTATAC CACCCTTCAG | 4920 |
| GATGAGGCCA TCAAGATATT CAATTCCCTG CAGCAACTGG AGTCCATGTC TGACCCAATT | 4980 |
| CCAATAATCC AGGGCATCCT ACAGACAGGG CATGACCTGC GACCTCTGCG GGACGAGCTG | 5040 |
| TACTGCCAGC TTATCAAACA GACCAACAAA GTGCCCCACC CCGGCAGTGT GGGCAACCTG | 5100 |
| TACAGCTGGC AGATCCTGAC ATGCCTGAGC TGCACCTTCC TGCCGAGTCG AGGGATTCTC | 5160 |
| AAGTATCTCA AGTTCCATCT GAAAAGGATA CGGGAACAGT TTCCAGGAAC CGAGATGGAA | 5220 |
| AAATACGCTC TCTTCACTTA CGAATCTCTT AAGAAAACCA AATGCCGAGA GTTTGTGCCT | 5280 |
| TCCCGAGATG AAATAGAAGC TCTGATCCAC AGGCAGGAAA TGACATCCAC GGTCTATTGC | 5340 |
| CATGGCGGCG GCTCCTGCAA GATCACCATC AACTCCCACA CCACTGCTGG GGAGGTGGTG | 5400 |
| GAGAAGCTGA TCCGAGGCCT GGCCATGGAG GACAGCAGGA ACATGTTTGC TTTGTTTGAA | 5460 |
| TACAACGGCC ACGTCCACAA AGCCATTGAA AGTCGAACCG TCGTAGCTGA TGTCTTAGCC | 5520 |
| AAGTTTGAAA AGCTGGCTGG CACATCCGAG GTTGGGGACC TGCCATGGAA ATTCTACTTC | 5580 |
| AAACTTTACT GCTTCCTGGA CACAGACAAC GTGCCAAAAG ACAGTGTGGA GTTTGCATTT | 5640 |
| ATGTTTGAAC AGGCCCACGA AGCGGTTATC CATCGCCACC ATCCAGCCCC GGAAGAAAAC | 5700 |
| CTCCACGTTC TTGCTGCCCT GCGACTCCAG TATCTCCAGC GGGATTATAC TCTGCACGCT | 5760 |
| GCCATCCCAC CTCTCGAAGA GGTTTATTCC CTGCAGAGAC TCAAGGCCCG CATCAGCCAG | 5820 |
| TCAACCAAAA CCTTCACCCC TTGTGAACGG CTGGAGAAGA GGCGGACGAG CTTCCTAGAG | 5880 |
| GGGACCCTGA GGCGGAGCTT CCGGACAGGA TCCGTGGTCC GGCAGAACGT CGAGGAGGAG | 5940 |
| CAGATGCTGG ACATGTGGAT TAAGGAAGAA GTCTCCTCTG CTCGAGCCAG TATCATTGAC | 6000 |
| AAGTGGAGGA AATTTCAGGG AATGAACCAG GAACAGGCCA TGGCCAAGTA CATGGCCTTG | 6060 |
| ATCAAGGAGT GGCCTGGCTA TGCCTCCACG CTGTTTCATC TGGAGTGCAA GGAAGGTGGC | 6120 |
| TTCCCTCAGG AACTCTGGTT GGGTGTCAGC GCGGACGCCG TCTCCGTCTA CAAGCGTGGA | 6180 |
| CAGGGAAGAC CACTGGAAGT CTTCCAGTAT GAACACATCC TCTCTTTTGG GGCACCCCTG | 6240 |
| GCGAATACGT ATAAGATCGT GGTCGATGAG AGGGAGCTGC TCTTTGAAAC CAGTGAGGTG | 6300 |
| GTGGATGTGG CCAAGCTCAT GAAAGCCTAC ATCAGCATGA TCGTGAACAA GCGCTACAGC | 6360 |
| ACGACACCCT CCGCCAGCAG CCAGCCCACC TCCACGTCAA GGCGGGACAG AGCCCACCTG | 6420 |
| TCTTTGCTAC CTGAACGCAC CACCCTCTGG CCTAGGCTGG CTCCAGTGTG CCATGCCCAG | 6480 |
| CCAAAACAAA CACAGAGCTG CCCAGGCTTT CTGGAAGCTT CTGGTCTGAG GGAGGTGTCT | 6540 |
| CCGAGGATCC TTTTGCCTGC CGCCTTCATT GATCCTGTAT TAAGCTGTCA ACTTTAACAG | 6600 |
| TCTCCACACT TTCCAAACCT TTACTACTCT TAGAGGACAC ATGCCTTAAA AAAGGAGGGG | 6660 |
| AGGAACCACG CTGCCACCAA AGCAGCCGGA AGTGCCTTAA CTTGTGGAAC CAACACTAAT | 6720 |
| CCACCGTAAC TGTGCTACTG AAGGCAACTG CCTTTCCCCC TTCTCGGGGA GACTTAACAG | 6780 |
| AGCGTGGAAG GGGGGCATTC TCTGTCAATG ATGCACTAAC CTCCCAACCT GATTTCCCCG | 6840 |
| AATCTGAGGG AAGGTGAGGG ACTCCCAAGG CGGATGGAGA GCTCGAGGGG ACAGTGTGTT | 6900 |
| TGAGCTGCAC TGCTGCCCCC AGCCTTTCTC ATGGAATGAC ATGAATCAAC TTTTTTCTTT | 6960 |
| GTTTCATCTT TTAAGTGTAC GTGCTTGCCT GTTCGTGCAT GTGTTCATAA ACTCAACACT | 7020 |
| TTAATCATCC TTTCATGAGC ATTAAAAACC AAAGGGAAAA AGGATGTGTA ATGGTGTACA | 7080 |
| CAGTCTGTAT ATTTTAATAA TGCAGAGCTA TAGTCTCAAT TGTTACTTTA TAAGGTGGTT | 7140 |
| TTATTAACAA ACCCAAATCC TGGATTTTCC TGTCTTTGCT GTATTTTGAA AAACACGTCT | 7200 |
| TGACTCCATT GTTTTACATG TAGCAAAGTC TGCCATCTGT GTCTGCTGTA TTATAAACAG | 7260 |
| ATAAGCAGCC TACAAGATAA CTGTATTTAT AAACCACTCT TCAACAGCTG GCTCCAGTGC | 7320 |
| TGGTTTTAGA ACAAGAATGA AGTCATTTTG GAGTCTTTCA TGTCTAAAAG ATTTAAGTTA | 7380 |
| AAAACAAAGT GTTACTTGGA AGGTTAGCTT CTATCATTCT GGATAGATTA CAGATATAAT | 7440 |
| AACCATGTTG ACTATGGGGG AGAGACGCTG CATTCCAGAA ACGTCTTAAC ACTTGAGTGA | 7500 |
| ATCTTCAAAG GACCCTGACA TTAAATGCTG AGGCTTTAAT ACACACATAT TTTATCCCAA | 7560 |
| GTTTATAATG GTGGTCTGAA CAAGGCACCT GTAAATAAAT CAGCATTTAT GACCAGAAGA | 7620 |
| AAAATAATCT GGTCTTGGAC TTTTTATTTT TATATGGAAA AGTTTTAAGG ACTTGGGCCA | 7680 |
| ACTAAGTCTA CCCACACGAA AAAAGAAATT TGCCTTGTCC CTTTGTGTAC AACCATGCAA | 7740 |
| AACTGTTTGT TGGCTCACAG AAGTTCTGAC AATAAAAGAT ACTAGCT | |

Seq ID NO: 207 Protein sequence:
Protein Accession #: NP_036466

| | |
|---|---|
| 1 11 21 31 41 51 | |
| \| \| \| \| \| \| | |
| MDNFFTEGTR VWLRENGQNF PSTVNSCAEG IVVFRTDYGQ VFTYKQSTIT HQKVTAMHPT | 60 |
| NEEGVDDMAS LTELHGGSIM YNLFQRYKRN QIYTYIGSIL ASVNPYQPIA GLYEPATMEQ | 120 |
| YSRRHLGELP PHIFAIANEC YRCLWKRYDN QCILISGESG AGKTESTKLI LKFLSVISQQ | 180 |
| SLELSLKEKT SCVERAILES SPIMEAFGNA KTVYNNNSSR FGKFVQLNIC QEGNIQGGRI | 240 |
| VDYLLEKNRV VRQNPGERNY HIFYALLAGL EHEEREEFYL STPENYHYLN QSGCVEDKTI | 300 |
| SDQESFREVI TAMDVMQFSK EEVREVSRLL AGILHLGNIE FITAGGAQYS FKTALGRSAE | 360 |
| LLGLDPTQLT DALTQRSMFL RGEEILTPLN VQQAVDSRDS LAMALYACCF EWVIKKINSR | 420 |
| IKGNEDFKSI GILDIFGFEN FEVNHFEQFN INYANEKLQE YFNKHIFSLE QLEYSREGLV | 480 |
| WEDIDWIDNG ECLDLIEKKL GLLALINEES HFPQATDSTL LEKLHSQHAN NHFYVKPRVA | 540 |
| VNNFGVKHYA GEYQYDYRGI LEKNRDTFRD DLLNLLRESR FDFIYDLFEH VSSRNNQDTL | 600 |
| KCGSKHRRPT VSSQFKDSLH SLMATLSSSN PFFVRCIKPN MQKMPDQFDQ AVVLNQLRYS | 660 |
| GMLETVRIRK AGYAVRRPFQ DFYKRYKVLM ENLALPEDYR GKCTSLLQLY DASNSEWQLG | 720 |
| KTKVFLRESL EQKLEKRREE EVSHAAMVIR AHVLGFLARK QYRKVLYCVV IIQKNYRAFL | 780 |
| LRRRFLHLKK AAIVFQKQLR GQIARRVYRQ LLAEKREQEE KKKQEEEEKK KREEEERERE | 840 |
| RERREAELRA QQEEETRKQQ ELEALQKSQK EAELTRELEK QKENKQVEEI LRLEKEIEDL | 900 |
| QRMKEQQELS LTEASLQKLQ ERRDQELRRL EEEACRAAQE FLESLNFDEI DECVRNIERS | 960 |
| LSVGSEFSSE LAESACEEKP NFNFSQPYPE EEVDEGFEAD DDAFKDSPNP SEHGHSDQRT | 1020 |
| SGIRTSDDSS EEDPYMNDTV VPTSPSADST VLLAPSVQDS GSLHNSSSGE STYCMPQNAG | 1080 |
| DLPSPDGDYD YDQDDYEDGA ITSGSSVTFS NSYGSQWSPD YRCSVGTYNS SGAYRFSSEG | 1140 |
| AQSSFEDSEE DFDSRFDTDD ELSYRRDSVY SCVTLPYFHS FLYMKGGLMN SWKRRWCVLK | 1200 |
| DETFLWFRSK QEALKQGWLH KKGGGSSTLS RRNWKKRWFV LRQSKLMYFE NDSEEKLKGT | 1260 |
| VEVRTAKEII DNTTKENGID IIMADRTFHL IAESPEDASQ WFSVLSQVHA STDQEIQEMH | 1320 |
| DEQANPQNAV GTLDVGLIDS VCASDSPDRP NSFVIITANR VLHCNADTPE EMHHWITLLQ | 1380 |
| RSKGDTRVEG QEFIVRGWLH KEVKNSPKMS SLKLKKRWFV LTHNSLDYYK SSEKNALKLG | 1440 |
| TLVLNSLCSV VPPDEKIFKE TGYWNVTVYG RKHCYRLYTK LLNEATRWSS AIQNVTDTKA | 1500 |
| PIDTPTQQLI QDIKENCLNS DVVEQIYKRN PILRYTHHPL HSPLLPLPYG DINLNLLKDK | 1560 |
| GYTTLQDEAI KIFNSLQQLE SMSDPIPIIQ GILQTGHDLR PLRDELYCQL IKQTNKVPHP | 1620 |
| GSVGNLYSWQ ILTCLSCTFL PSRGILKYLK FHLKRIREQF PGTEMEKYAL FTYESLKKTK | 1680 |
| CREFVPSRDE TEALIHRQEM TSTVYCHGGG SCKITINSHT TAGEYVEFLI RGLANEDSRN | 1740 |
| MFALFEYNGH VDKAIESRTV VADVLAKFEK LAATSEVGDL PWKFYFKLYC FLDTDNVPKD | 1800 |
| SVEFAFMFEQ AHEAVIHGHH PAPEENLQVL AALRLQYLQG DYTLHAAIPP LEEVYSLQRL | 1860 |
| KARISQSTKT FTPCERLEKR RTSFLEGTLR RSFRTCSVVR QKVEEEQMLD MWIKEEVSSA | 1920 |
| RASIIDKWRK FQGMNQEQAN AKYMALIKEW PGYGSTLFDV ECKEGGFPQE LWLGVSADAV | 1980 |
| SVYKRGEGRP LEVFQYEHIL SFGAPLANTY KIVVDERELL PETSEVVDVA KLMKAYISMI | 2040 |
| VKKRYSTTRS ASSQGSSR | |

Seq ID NO: 208 DNA sequence:
Nucleic Acid Accession #: XM_059761.1
Coding sequence: 124-925 (underlined sequences correspond to start and stop codons)

| | |
|---|---|
| 1 11 21 31 41 51 | |
| \| \| \| \| \| \| | |
| CGAAGATCTA TCCAAAATCA AGAAGCCTTT GATTTAGATG TTGCTGTAAA AGAAAATAAA | 60 |
| GATGATCTCA ATCATGTGGA TTTGAATGTG TGTACAAGCT TTTCGGGCCC GGGTAGGAGT | 120 |
| CCCATGGCTC TTATGGAAGT TAACCTATTA AGTCCCTTTA TGGTGCCTTC AQAACCAATT | 180 |
| TCTCTGAGCC ACACACTCAA GAAAGTGGAA TATGATCATG GAAAACTCAA CCTCTATTTA | 240 |
| GATTCTGTAA ATGAAACCCA GTTTTCTGTT AATATTCCTC CTGTGAGAAA CTTTAAAGTT | 300 |
| TCAAATACCC AAGATGCTTC AGTGTCCATA GTGGATTACT ATGAGCCAAG GAGACAGGCG | 360 |
| GTGASAAGTT ACAACTCTGA AGTGAAGCTG TCCTCCTGTG ACCTTTGCAC TGATGTCCAG | 420 |
| GGCTGCCGTC CTTGTGAGGA TGGAGCTTCA GGCTCCCATC ATCACTCTTC AGTCATTTTT | 480 |
| ATTTTCTGTT TCAAGCTTCT GTACTTTATG GAACTTTGGC TGTGATTTAT TTTTAAAGGA | 540 |
| CTCTGTGTAA CACTAACATT TCCAGTAGTC ACATCTGATT CTTTTGTTTT CGTAGAAGAA | 600 |
| TACTGCTTCT ATTTTGAAAA AAGAGTTTTT TTTCTTTCTA TGGGGTTGCA GGGATCGTGT | 660 |
| ACAACAGGTC CTACCATGTA TAGCTGCATA GATTTCTTCA CCTGATCTTT GTGTGGAACA | 720 |
| TCAGAATGAA TGCAGTTGTG TGTCTATATT TTCCCCTCTC AAAATCTTTT AGAATTTTTT | 780 |
| TGGAGGTGTT TGTTTTCTCC AGAATAAAGG TATTACTTTA C | |

Seq ID NO: 209 Protein sequence:
Protein Accession #: XP_059761.1

| | |
|---|---|
| 1 11 21 31 41 51 | |
| \| \| \| \| \| \| | |
| MALMEVNLLS GFMVPSEAIS LSETVKKVEY DHGKLNLYLD SVNETQFCVN IPAVRNFKVS | 60 |
| NTQDASVSIV DYYEPRRQAV RSYNSEVKLS SCDLCSDVQG CRPCEDGASC SHHHSSVIFI | 120 |
| FCFKLLYFME LWL | |

Seq ID NO: 210 DNA sequence:
Nucleic Acid Accession #: NM_015472
Coding sequence: 258-1460 (underlined sequences correspond to start and stop codons)

| | |
|---|---|
| 1 11 21 31 41 51 | |
| \| \| \| \| \| \| | |
| CACACACTCC TCTACAACAC CACAGACTCC CAAACACAAG CCCTTATATT GACTCATTTC | 60 |
| AGCTCACATC CTGGCGACTC TCAAGACAGA AACCTCAGAG TGACTAAAAT CTCCATAATG | 120 |
| ACAACACATG TACATTCACT ATCTATTTTG GCATTTTCCC CAATACATCT CTGCTCATCT | 180 |
| GACTCTTATC TTGGCATCTG CTTCCTCCTG GATCTGAACT GACCCATAAG CCACGCTTAC | 240 |
| TGCTCATTTT CCAGAAGATG AATCCCCCCT CCGCGCCCCC TCCCCTCCCG CCGCCTGGGC | 300 |
| ACCAAGTGAT CCACCTCACG CAGCACCTAG ACACAGACCT CGAAGCCCTC TTCAACTCTG | 360 |
| TCATGAATCC GAAGCCTAGC TCGTGCCGCA ACAACATCCT GCCGGAGTCT TTCTTTAAGG | 420 |
| AGCCTCATTC CGGCTCGCAC TCGCGCCAGT CCACCACCGA CTCGTCGGCC CCCCACCCGG | 480 |
| GGCCTCGACT GGCTCGGGGT GCCCAGCATG TCCGCTCGCA CTCGTCGCCC GCGTCCCTGC | 540 |
| AGCTGGGCAC CCCCGCGGGT GCTCCGGCTA CCCCCGCGCA GCAGCACGCC CACCTCCCCC | 600 |
| ACCACTCCTA CGACGTGACC GACGAGCTGC CACTGCCCCC CCGCTCGGAC ATCACCTTCA | 660 |
| CGGCCACTGC CCAGAGGTAC TTCCTCAATC ACATAGAAAA AATCACCACA TGCCAAGACC | 720 |
| CTAGGAAGGC GATGAATCAG CCTCTGAATC ATATGAACCT CCACCCTGCC GTCAGTTCCA | 780 |
| CACCAGTGCC TCAGAGGTCC ATCCCACTAT CCCACCCAAA TCTCGTCATG AATCACCAAC | 840 |
| ACCAGCAGCA GATGGCCCCC ACTACCCTCA GCCAGCAGAA CCACCCCACT CAGAACCCAC | 900 |
| CCGCAGGGCT CATGAGTATG CCCAATCCCC TCACCACTCA GCAGCAGCAG CAGCACAAAC | 960 |
| TCCGGCTTCA CAGAATCCAG ATGGAGAGAG AAAGGATTCG AATGCCCCAA GAGGAGCTCA | 1020 |
| TGACGCACCA ACCTCCCCTC TGTCGACAGC TCCCCATCCA AGCTGAGACT CTTGCCCCAG | 1080 |
| TTCAGCCTGC TGTCAACCCA CCCACGATGA CCCCACACAT CAGATCCATC ACTAATAATA | 1140 |
| CCTCACATCC TTTCCTCAAT CGAGGGCCAT ATCATTCCAG GGAGCAGAGC ACTGACAGTG | 1200 |
| GCCTGGGGTT AGGGTGCTAC ACTCTCCCCA CAACTCCCGA GGACTTCCTC ACCAATGTGC | 1260 |
| ATGACATGGA TACAGGACAA AACGCAGGAC AAACACCCAT GAACATCAAT CCCCAACAGA | 1320 |
| CCCGTTTCCC TGATTTCCTT CACTGTCTTC CACCAACAAA CGTTGACTTA CGAACTTTGC | 1380 |
| AATCTGAAGA CCTGATCCCC CTCTTCAATG ATGTAGACTC TGCTCTCAAC AAAAGTCACC | 1440 |
| CCTTTCTAAC CTCGCTGTAA TCACTACCAT TGTAACTTGG ATGTAGCCAT GACCTTACAT | 1500 |
| TTCCTCGCCC TCTTGCAAAA ACTGATGCAG CACAGCAAGT CTCCAGGTGC ACCACTTCCC | 1560 |
| GCCTCCATGA CTCGTGCTCC CTCCTTTTTA TGTTGCCAGT TTAATCATTC CCTGGTTTTG | 1620 |
| ATTCAGAGTA ACTTAAGTTA AACATAAATA AATATTCTAT TTTCATTTTC | |

Seq ID NO: 211 Protein sequence:
Protein Accession #: NP_056287.1

| | |
|---|---|
| 1 11 21 31 41 51 | |
| \| \| \| \| \| \| | |
| MNPASAPPPL PPPGQQVIHV TQDLDTDLEA LFNSVMNPKP SSWRKKILPE SFFKEPDSGS | 60 |
| HSRQSSTDSS GGHPGPRLAG GAQHVRSHSS PASLQLGTGA CAAGSPAQQH AHLRQQSYDV | 120 |
| TDELPLPPGW EMTFTATGQR YFLNHIEKIT TWQDPRKAMN QPLNHMNLHP AVSSTPVPQR | 180 |
| SMAVSQPNLV MNHQHQQQMA PSTLSQQNHP TQNPPAGLMS MPNALTTQQQ QQQKLRLQRI | 240 |
| QMERERIRMR QEELMRQEAA LCRQLPMEAE TLAPVQAAVN PPTMTPDMRS ITNNSSDPFL | 300 |
| NGGPYHSREQ STDSGLGLGC YSVPTTPEDF LSNVDEMDTC ENAGQTPMNI NPQQTRFPDF | 360 |
| LDCLPGTNVD LGTLESEDLI PLFNDVESAL NKSEPFLTWL | |

| | |
|---|---|
| Seq ID NO: 212 DNA sequence: | |
| Nucleic Acid Accession #: NM_018174 | |
| Coding sequence: 176-2194 (underlined sequences correspond to start and stop codons) | |
| CATCTCCCCC AACCTGCGGG TCGTGTTCTT CAACGCCTGC GAGGCCGCGT CGCGGCTGGC | 60 |
| GCGCGGCGAG GATGAGCCGG AGCTGGCGCT GAGCCTCCTG CCCCAGCTGG GCATCACGCC | 120 |
| TCTGCCACTC AGCCGCGGCC CCGTGCCACC CAAACCCACC GTGCTCTTCG AGAAGATGGG | 180 |
| CGTCGGCCGG CTGGACATGT ATGTGCTGCA CCCGCCCTCC GCCGGCGCCG AGCGCACGCT | 240 |
| GGCCTCTGTG TGCGCCCTGC TGGTGTGGCA CCCCGCCGGC CCCGGCGAGA AGGTCGTGCC | 300 |
| CGTGCTGTTC CCCGGTTGCA CCCCGCCCGC CTGCCTCCTG CACGGCCTGG TCCGCCTGCA | 360 |
| GCACTTGACG TTCCTGCGAG AGCCCCTCCT GACGCCCCAG GACCTGGAGG GGCCGGGGCG | 420 |
| AGCCGACACC AAAGAGAGCG TCGGCTCCCG GGACAGCTCG AAGAGAGACG GCCTCCTGGC | 480 |
| CACCCACCCT AGACCTGGCC AGGACCGCCC TGGCGTGGCC CGCAAGCACC CAGCACGGGC | 540 |
| TGAGGCCCCA CGCAACACTG AGAAAGAAGC CAAGACCCCC CGGGAGTTGA AGAAAGACCC | 600 |
| CAAACCGAGT GTCTCCCGGA CCCAGCCGCG GGAGGTGCGC CGCGCAGCCT CTTCTSTGCC | 660 |
| CAACCTCAAG AAGACGAATG CCCAGGCGGC ACCCAACCCC CGCAAAGCGC CCAGCACGTC | 720 |
| CCACTCTGCC TTCCCGCCGG TGGCAAATGG ACCCCGCAGC CCGCCCAGCC TCCGATGTGG | 780 |
| AGAAGCCAGC CCCCCCAGTG CAGCCTGCGG CTCTCCGGCC TCCCAGCTGG TGGCCACGCC | 840 |
| CAGCCTGGAG CTGGGGCCCA TCCCACCCGG GGArnAGAAC GCACTGGAGC TGCCTTTGGC | 900 |
| CGCCACCTCA ATCCCAAGSC CACCCACACC CTCCCCTGAG TCCCACCGGA GCCCCGCAGA | 960 |
| GGGCAGCGAG CGGCTGTCGC TGAGCCCACT GCGGGGCGGC GACGCCGGGC CAGACGCCTC | 1020 |
| ACCCACAGTG ACCACACCCA CGGTGACCAC CCCCTCACTA CCCGCAGAGC TGGGCTCCCC | 1080 |
| GCACTCGACC GAGGTCCACG AGTCCCTGTC CGTGTCCTTT GAGCAGGTGC TGCCGCCATC | 1140 |
| CGCCCCCACC ACTGAGGCTG GGCTGACCCT CCCGCTGCGT GGCCCCCGGG CGCGGCGCTC | 1200 |
| GGCTTCCCCA CACGATGTGG ACCTGTGCCT GGTGTCACCC TCTCAATTTG AGCATCGCAA | 1260 |
| GGCGGTCCCA ATGGCACCGG CACCTGCGTC CCCCGGCAGC TCGAATGACA GCAGTGCCCG | 1320 |
| GTCACAGGAA CGGGCAGGTG GGCTGGGGGC CGAGGACACG CCACCCACAT CGGTCAGCGA | 1380 |
| GTCCCTGCCC ACCCTGTCTG ACTCGGATCC CGTGCCCCTG GCCCCCGGTG CGGCAGACTC | 1440 |
| AGACGAAGAC ACAGAGGGCT TTGGAGTCCC TCGCCACGAC CCTTTGCCTG ACCCCCTCAA | 1500 |
| GGTCCCCCCA CCACTGCCTG ACCCATCCAG CATCTGCATG STGGACCCCG AGATGCTGCC | 1560 |
| CCCCAAGACA GCACGGCAAA CGGAGAACGT CAGCCGCACC CGGAAGCCCC TGGCCCGCCC | 1620 |
| CAACTCACGC GCTGCCGCCC CCAAAGCCAC TCCAGTGGCT GCTGCCAAAA CCAAGGGGCT | 1680 |
| TGCTGGTGGG GACCGTGCCA GCCGACCACT CAGTGCCCGG AGTGAGCCCA GTGAGAAGGG | 1740 |
| AGGCCGGGCA CCCCTGTCCA GAAAGTCCTC AACCCCCAAG ACTGCCACTC GAGGCCCGTC | 1800 |
| GGGGTCAGCC AGCAGCCGGC CCGGGGTGTC AGCCACCCCA CCCAAGTCCC CGGTCTACCT | 1860 |
| GGACCTGGCC TACCTGCCCA GCGGGAGCAG CGCCCACCTG GTGGATGAGG AGTTCTTCCA | 1920 |
| GCGCGTGCGC GCGCTCTGCT ACGTCATCAG TGGCCAGGAC CAGCGCAAGG AGGAAGGCAT | 1980 |
| GCGGGCCGTC CTGGACGCGC TACTGGCCAG CAAGCAGCAT TGGGACCGTG ACCTGCAGGT | 2040 |
| GACCCTGATC CCCACTTTCG ACTCGGTGGC CATGCATACG TGGTACGCAG AGACGCACGC | 2100 |
| CCGGCACCAG GCGCTGGGCA TCACGGTGTT GGGCAGCAAC GGCATGGTGT CCATGCAGGA | 2160 |
| TGACGCCTTC CCGGCCTGCA AGGTGGAGTT CTAGCCCCAT CGCCGACACG CCCCCCACTC | 2220 |
| AGCCCAGCCC GCCTGTCCCT AGATTCAGCC ACATCAGAAA TAAACTGTGA CTACACTTG | |
| Seq ID NO: 213 Protein sequence: | |
| Protein Accession #: NP_060644.1 | |
| MGVGRLDMYV LHPPSAGAER TLASVCALLV WHPAGPGEKV VRVLFPGCTP PACLLDGLVR | 60 |
| LQHLRFLREP VVTPQDLEGP GRAESKESVG SRDSSKREGL LATHPRPGQE RPGVARKEPA | 120 |
| RAEAPRKTEK EAKTPRELKK DPKPSVSRTQ PREVRRAASS VPNLKKTNAQ AAPKPRKAPS | 180 |
| TSHSGFPPVA NGPRSPPSLR CGEASPPSAA CGSPASQLVA TPSLELGPIP AGEEKALELP | 240 |
| LAASSIPRPR TPSPESHRSP AEGSERLSLS PLRGGEAGPD ASPTVTTPTV TTPSLPAEVG | 300 |
| SPHSTEVDES LSVSFEQVLP PSAPTSEAGL SLPLRGPRAR RSASPHDVDL CLVSPCEFEH | 360 |
| RKAVPMAPAP ASPGSSNDSS ARSQERAGGL GAEETPPTSV SESLPTLSDS DPVPLAPGAA | 420 |
| DSDEDTEGFG VPRHDPLPDP LKVPPPLPDP SSICMVDPEM LPPKTARQTE NVSRTRKPLA | 480 |
| RPNSRAAAPK ATPVAAAKTK GLAGGDRASR PLSARSEPSE KGGRAPLSRK SSTPKTATRG | 540 |
| PSGSASSRPG VSATPPKSPV YLDLAYLPSG SSAHLVDEEF FQRVRALCYV ISGQDQRKEE | 600 |
| GMRAVLDALL ASKQHWDRDL QVTLIPTFDS VAMHTWYAET HARHQALGIT VLGSNGMVSM | 660 |
| QDDAFPACKV EF | |
| Seq ID NO: 214 DNA sequence: | |
| Nucleic Acid Accession #: NM_002019.1 | |
| Coding sequence: 250-4266 (underlined sequences correspond to start and stop codons) | |

| | |
|---|---|
| 1 11 21 31 41 51 | |
| \| \| \| \| \| \| | |
| GCGGACACTC CTCTCGGCTC CTCCCCGGCA GCGGCGGCGG CTCGGAGCGG GCTCCGGGGC | 60 |
| TCGGGTGCAG CGGCCAGCGG GCCTGGCGGC GAGGATTACC CGGGGAAGTG GTTGTCTCCT | 120 |
| GGCTGGAGCC GCGAGACGCG CGCTCAGGGC CCGGGGCCGC CGGCGCCGAA CGAGAGGACG | 180 |
| GACTCTGGCG CCCGCGTCCT TGGCCGGGGG AGCGCGGGCA CCGGGCGAGC AGGCCGCGTC | 240 |
| GCGCTCACCA TGGTCAGCTA CTGGGACACC GGGGTCCTGC TCTGCGCGCT GCTCAGCTGT | 300 |
| CTGCTTCTCA CAGGATCTAG TTCAGGTTCA AAATTAAAAG ATCCTGAACT CAGTTTAAAA | 360 |
| GGCACCCAGC ACATCATGCA AGCAGGCCAG ACACTGCATC TCCAATGCAG GGGGGAAGCA | 420 |
| GCCCATAAAT GGTCTTTGCC TGAAATGGTG AGTAAGGAAA GCGAAAGGCT GAGCATAACT | 480 |
| AAATCTGCCT GTGGAAGAAA TGGCAAACAA TTCTGCAGTA CTTTAACCTT GAACACAGCT | 540 |
| CAACCAAACC ACACTGQCTT CTACAGCTGC AAATATCTAG CTGTACCTAC TTCAAAGAAG | 600 |
| AAGGAAACAG AATCTGCAAT CTATATATTT ATTACTGATA CAGGTAGACC TTTCGTAGAG | 660 |
| ATGTACAGTG AAATCCCCGA AATTATACAC ATGACTGAAG GAAGGGAGCT CGTCATTCCC | 720 |
| TGCCGGGTTA CGTCACCTAA CATCACTGTT ACTTTAAAAA AGTTTCCACT TGACACTTTC | 780 |
| ATCCCTGATG CAAAACGCAT AATCTGGGAC AGTAGAAAGG GCTTCATCAT ATCAAATGCA | 840 |
| ACGTACAAAG AAATAGGGCT TCTGACCTGT GAAGCAACAC TCAATGGGCA TTTGTATAAG | 900 |
| ACAAACTATC TCACACATCG ACAAACCAAT ACAATCATAG ATGTCCAAAT AAGCACACCA | 960 |
| CGCCCAGTCA AATTACTTAG AGGCCATACT CTTGTCCTCA ATTGTACTGC TACCACTCCC | 1020 |
| TTGAACACGA GAGTTCAAAT GACCTGGAGT TACCCTCATC AAAAAAATAA GAGAGCTTCC | 1080 |
| GTAACCCCAC GAATTGACCA AAGCAATTCC CATGCCAACA TATTCTACAG TGTTCTTACT | 1140 |
| ATTGACAAAA TGCAGAACAA ACACAAAGGA CTTTATACTT GTCGTGTAAG GAGTGGACCA | 1200 |
| TCATTCAAAT CTGTTAACAC CTCAGTGCAT ATATATGATA AAGCATTCAT CACTGTCAAA | 1260 |
| CATCGAAAAC AGCAGGTGCT TGAAACCGTA GCTGGCAAGC CGTCTTACCG CCTCTCTATG | 1320 |
| AAAGTGAAGG CATTTCCCTC CCCGGAAGTT GTATGGTTAA AAGATGGGTT ACCTGCGACT | 1380 |
| CACAAATCTG CTCGCTATTT GACTCGTGGC TACTCGTTAA TTATCAAGGA CGTAACTGAA | 1440 |
| GAGGATGCAG GGAATTATAC AATCTTGCTC AGCATAAAAC AGTCAAATGT GTTTAAAAAC | 1500 |
| CTCACTGCCA CTCTAATTGT CAATGTGAAA CCCCAGATTT ACGAAAAGGC CGTGTCATCG | 1560 |
| TTTCCAGACC CGGCTCTCTA CCCACTGGGC AGCAGACAAA TCCTGACTTG TACCGCATAT | 1620 |
| GGTATCCCTC AACCTACAAT CAAGTGGTTC TGGCACCCCT CTAACCATAA TCATTCCGAA | 1680 |
| GCAAGGTGTG ACTTTTGTTC CAATAATGAA GAGTCCTTTA TCCTGGATGC TGACAGCAAC | 1740 |
| ATCGCAAACA GAATTGACAG CATCACTCAG CGCATGGCAA TAATACAACG AAAGAATAAG | 1800 |
| ATGGCTAGCA CCTTGGTTGT GGCTGACTCT AGAATTTCTG CAATCTACAT TTGCATAGCT | 1860 |
| TCCAATAAAG TTGCCACTGT GGGAAGAAAC ATAAGCTTTT ATATCACAGA TGTGCCAAAT | 1920 |
| CGGTTTCATG TTAACTTGGA AAAAATGCCG ACGGAAGCAG AGCACCTGAA ACTGTCTTGC | 1980 |
| ACAGTTAACA AGTTCTTATA CACAGACCTT ACTTGGATTT TACTGCGGAC AGTTAATAAC | 2040 |
| AGAACAATGC ACTACAGTAT TAGCAAGCAA AAAATGGCCA TCACTAAGGA GCACTCCATC | 2100 |
| ACTCTTAATC TTACCATCAT GAATGTTTCC CTGCAAGATT CACCCACCTA TGCCTGCAGA | 2160 |
| CCCAGGAATG TATACACACG GGAAGAAATC CTCCAGAAGA AAGAAATTAC AATCAGAGAT | 2220 |
| CAGGAAQCAC CATACCTCCT GCGAAACCTC AGTGATCACA CAGTGGCCAT CACCAGTTCC | 2280 |
| ACCACTTTAG ACTGTCATGC TAATGCTGTC CCCGAGCCTC ACATCACTTG GTTTAAAAAC | 2340 |
| AACCACAAAA TACAACAAGA GCCTGGAATT ATTTTAGGAC CAGGAAGCAG CACGCTGTTT | 2400 |
| ATTCAAAGAG TCACAGAAGA GGATGAAGGT GTCTATCACT GCAAAGCCAC CAACCAGAAG | 2460 |
| GGCTCTGTGG AAAGTTCAGC ATACCTCACT GTTCAAGGAA CCTCGCACAA CTCTAATCTG | 2520 |
| GAGCTGATCA CTCTAACATG CACCTGTGTC GCTCCGACTC TCTTCTGGCT CCTATTAACC | 2580 |
| CTCCTTATCC GAAAAATGAA AAGGTCTTCT TCTGAAATAA AGACTGACTA CCTATCAATT | 2640 |
| ATAATGGACC CAGATGAAGT TCCTTTGGAT GAGCAGTGTG AGCGGCTCCC TTATGATCCC | 2700 |
| AGCAAGTGGC ACTTTGCCCG GGAGAGACTT AAACTGGGCA AATCACTTGG AAGAGGGGCT | 2760 |
| TTTGGAAAAG TGGTTCAAGC ATCAGCATTT GGCATTAAGA AATCACCTAC GTGCCGCACT | 2820 |
| GTGGCTGTGA AAATGCTGAA AGAGGGGGCC ACGCCCACCC AGTACAAAGC TCTGATGACT | 2880 |
| GAGCTAAAAA TCTTGACCCA CATTGGCCAC CATCTGAACG TGGTTAACCT GCTGGGAGCC | 2940 |
| TGCACCAACC AAGGAGGGCC TCTGATGCTG ATTGTTGAAT ACTCCAAATA TGGAAATCTC | 3000 |
| TCCAACTACC TCAAGAGCAA ACGTGACTTA TTTTTTCTCA ACAAGGATGC AGCACTACAC | 3060 |
| ATGGAGCCTA AGAAAGAAAA AATGGAGCCA CGCCTGGAAC AAGGCAAGAA ACCAAGACTA | 3120 |
| GATAGCGTCA CCAGCACCCA AAGCTTTGCG AGCTCCGGCT TTCAGGAAGA TAAAAGTCTG | 3180 |
| AGTGATGTTG AGGAAGAGGA GGATTCTGAC GGTTTCTACA AGGAGCCCAT CACTATGGAA | 3240 |
| GATCTGATTT CTTACAGTTT TCAAGTGGCC AGAGGCATGG AGTTCCTGTC TTCCAGAAAG | 3300 |
| TGCATTCATC GGCACCTGGC AGCCAGAAAC ATTCTTTTAT CTGAGAACAA CGTGGTGAAG | 3360 |
| ATTTCTCATT TTGGCCTTGC CCGGGATATT TATAAGAACC CCGATTATGT GAGAAAAGGA | 3420 |
| GATACTCGAC TTCCTCTGAA ATGGATGCCT CCCGAATCTA TCTTTGACAA AATCTACAGC | 3480 |
| ACCAAGAGCG ACGTGTGGTC TTACGGAGTA TTGCTGTGGG AAATCTTCTC CTTAGGTGGG | 3540 |
| TCTCCATACC CACGAGTACA AATGGATGAG GACTTTTGCA GTCGCCTGAG GGAAGCCATG | 3600 |
| AGGATGAGAG CTCCTGAGTA CTCTACTCCT GAAATCTATC ACATCATGCT GGACTGCTGG | 3660 |
| CACAGAGACC CAAAACAAAC GCCAACATTT CCAGAACTTG TGGAAAAACT AGGTGATTTG | 3720 |
| CTTCAAGCAA ATGTACAACA GGATGGTAAA GACTACATCC CAATCAATGC CATACTGACA | 3780 |
| GGAAATAGTG GGTTTACATA CTCAACTCCT GCCTTCTCTG AGGACTTCTT CAAGGAAAGT | 3840 |
| ATTTCAGCTC CGAAGTTTAA TTCAGGAAGC TCTGATGATG TCAGATATGT AAATCCTTTC | 3900 |
| AAGTTCATGA GCCTGGAAAG AATCAAAACC TTTGAAGAAC TTTTACCCAA TGCCACCTCC | 3960 |
| ATGTTTGATG ACTACCAGGG CGACACCAGC ACTCTGTTGG CCTCTCCCAT GCTGAAGCCC | 4020 |
| TTCACCTGCA CTGACAGCAA ACCCAAGGCC TCGCTCAAGA TTGACTTGAC AGTAACCAGT | 4080 |
| AAAAGTAAGG AGTCGGGGCT GTCTGATGTC AGCAGGCCCA GTTTCTGCCA TTCCAGCTGT | 4140 |
| GGGCACGTCA GCGAAGGCAA GCGCAGGTTC ACCTACCACC ACGCTGAGCT GGAAAGGAAA | 4200 |
| ATCGCGTGCT GCTCCCCGCC CCCAGACTAC AACTCGGTGG TCCTGTACTC CACCCCACCC | 4260 |
| ATCTAGAGTT TGACACGAAG CCTTATTTCT AGAAGCACAT GTGTATTTAT ACCCCCAGGA | 4320 |
| AACTAGCTTT TGCCAGTATT ATGCATATAT AAGTTTACAC CTTTATCTTT CCATGGGAGC | 4380 |
| CAGCTGCTTT TTGTCATTTT TTTAATAGTG CTTTTTTTTT TTGACTAACA AGAATGTAAC | 4440 |
| TCCAGATAGA CAAATACTCA CAAGTGAACA ACACTACTGC TAAATCCTCA TGTTACTCAG | 4500 |
| TGTTAGAGAA ATCCTTCCTA AACCCAATGA CTTCCCTGCT CCAACCCCCG CCACCTCAGG | 4560 |
| GCACGCAGGA CCAGTTTGAT TGAGGAGCTG CACTCATCAC CCAATGCATC ACGTACCCCA | 4620 |
| CTGGGCCAGC CCTGCAGCCC AAAACCCAGG GCAACAAGCC CGTTAGCCCC AGGGGATCAC | 4680 |
| TGGCTGGCCT CACCAACATC TCGGGAGTCC TCTAGCAGGC CTAAGACATG TGAGGAGCAA | 4740 |
| AAGGAAAAAA AGCAAAAAGC AAGGGAGAAA AGAGAAACCG GGAGAAGGCA TGACAAAGAA | 4800 |
| TTTGAGACGC ACCATGTGGG cACGGAGGGG GACGGGGCTC AGCAATCCCA TTTCAGTGGC | 4860 |
| TTCCCACCTC TGACCCTTCT ACATTTCACC GCCCACCCAC CACCACATGG ACAGCCATGA | 4920 |
| GGGGACATTT TCTGGATTCT GGGAGGCAAG AAAAGGACAA ATATCTTTTT TGGAACTAAA | 4980 |
| GCAAATTTTA GACCTTTACC TATGCAACTG CTTCTATCTC CATTCTCATT CGTGGCATGT | 5040 |
| TTTGATTTGT ACCACTGACG CTCGCACTCA ACTCTCAGCC CATACTTTTG CCTCCTCTAG | 5100 |
| TAAGATGCAC TGAAAACTTA GCCAGAGTTA GGTTGTCTCC AGGCCATGAT GGCCTTACAC | 5160 |
| TGAAAATCTC ACATTCTATT TTCCCTATTA ATATATACTC CACACACTTA ACTCAATTTC | 5220 |
| TTGCTATTAT TCTGTTTTCC ACAGTTAGTT GTCAAACAAA CCTCACAAGA ATCAAAATCC | 5280 |
| ACTCCTGAGG AGAGTTTTCT CCATATCAAA ACGAGGGCTG ATGGAGGAAA AAGCTCAATA | 5340 |
| ACCTCAAGGC AAGACCCCGT CTCTATACCA ACCAAACCAA TTCACCAACA CACTTCCCAC | 5400 |
| CCAAAACACA GGAAGTCAGT CACGTTTCCT TTTCATTTAA TGGGGATTCC ACTATCTCAC | 5460 |
| ACTAATCTCA AAGGATGTGG AAGACCATTA GCTGCCGCAT ATTAACCACT TTAAGCTCCT | 5520 |
| TGAGTAAAAA GGTGGTATGT AATTTATGCA ACCTATTTCT CCACTTCCCA CTCAGCATAT | 5580 |
| TACTTAATCA CCCATCACTA CAAGAAAACC CCATTTTCAA CTCCTTTGAA ACTTCCCTCC | 5640 |
| GGTCTGAGCA TGATGGGAAT AGGGAGACAG GGTAGGAAAG GGCGCCTACT CTTCAGGGTC | 5700 |
| TAAACATCAA CTCCCCCTTG CATCCCTAAC CTCCCTCTCT TTCATCCTAT TTATCCAACT | 5760 |
| TACCCTCTAT CTATTTACCA TGCCCCTACT CTTCACCCTC TAAACATCAA CTCCCCCTTG | 5820 |
| CATCCCTAAC CTCCCTCTCT TTCATCCTAT TTATCCAACT TACCCTCTAT CTATTTAGGA | 5880 |
| TGTCTGCACC TTCTCCACCC ACTCACAAGC TGCAGACGCA ACACTCCATT GCTCCTTCTT | 5940 |
| GCCCACAACA CTATCCTTCC TTTTATCCAT CTAATTTAAC TCTACAACCT CACCTCTAAG | 6000 |
| TAACCGAAGA ATGTATGCCT CTGTTCTTAT GTGCCACATC CTTGTTTAAA GGCTCTCTGT | 6060 |
| ATCAACACAT GCCACCCTCA TCACCACATT CCCTAGTCAG CCTACTGGCT CCTGCCAGCG | 6120 |
| CCTTTTCTCC AAGACTCACT ACCCACAACA CAGGACTGGC ACAGTCCTCT CCACCAACAT | 6180 |
| CTAAATCCAA ACAAAACCAC GCTAGAGCCA CAACACACCA CAAATCTTTC TTCTTCCTCT | 6240 |
| TCTTTACACA TACCCAAACC ACCTCTCACA CCTCCCAATT TTATAAATCA CCTAACTCCA | 6300 |
| AGGAGGTTAA ACTCAGAAAA AAGAAGACCT CAGTCAATTC TCTACTTTTT TTTTTTTTTT | 6360 |
| TCCAAATCAC ATAATACCCC AGCAAATAGT GATAACAAAT AAAACCTTAG CTGTTCATGT | 6420 |
| CTTCATTTCA ATAATTAATT CTTAATCATT AACAGACCAT AATAAATACT CCTTTTCAAC | 6480 |
| ACAAAACCAA AACCATTAGA ATTGTTACTC AGCTCCTTCA AACTCAGGTT TGTAGCATAC | 6540 |
| ATCACTCCAT CCATCACTCA AACAATCGTT CCATCTCGAG TCTTAATGTA GAAAGAAAAA | 6600 |
| TGCACACTTC TAATAATCAC CTACTTACAA ACTCCTTCTT CATTAAAATA CCACTCAAAA | 6660 |
| TTGAAACATG AATTAACTGA TAATATTCCA ATCATTTGCC ATTTATGACA AAAATGGTTG | 6720 |
| GCACTAACAA AGAACCACCA CTTCCTTTCA GACTTTCTCA CATAATGTAC CTCCAACAGT | 6780 |
| CTCCCTCCAA TCCGCCTCAA ACCATCTCCA ACTCTGTCTC TTGTCACTCC AACAACTCAC | 6840 |
| ACCGAGATGT TAATTTTAGG GACCCGTGCC TTGTTTCCTA GCCCACAAGA ATGCAAACAT | 6900 |
| CAAACACATA CTCCCTACCC TCATTTAAAT TCATTAAACC ACCACTGCAT CTTTCCCCCA | 6960 |
| CACTCCTCTA ACTCTCTCTC TGTGTCTGTC TGTGTGTGTG TGTGTGTGTC TGTCGCTGTG | 7020 |
| CGTGTATCTG TGTTTTCTCC ATAACTATTT AAGGAAACTG GAATTTTAAA GTTACTTTTA | 7080 |
| TACAAACCAA CAATATATCC TACACATATA ACACACACAT CCTTTCCTCC TATATTTCTA | 7140 |
| CTCATCATCA ATGTATTTTC TATACCATCT TCATATAATA TACTTAAAAA TATTTCTTAA | 7200 |
| TTCCGATTTC TAATCCTACC AACTTAATTC ATAAACTTCG CAACTGCTTT TATGTTCTCT | 7260 |
| CTCCTTCCAT AAATTTTTCA AAATACTAAT TCAACAAACA AAAACCTCTT TTTTTTCCTA | 7320 |
| AAATAAACTC AAATTTATCC TTCTTTAGAC CAGACAAAAA TTAACAAAAA CTTTCAAATC | 7380 |
| CTCTCAAAAA ATTCCTAAAT ATTTTCAATC CAAAACTAAA TGTTACTTTA CCTCATTGTA | 7440 |
| TCCCCTTTTC CAACCTTTCA CTTTTTGTTT CTTTTACCTA TTTCACAACT CTCTAAATTC | 7500 |
| CCAATAATTC CTCTCCATCA AAATCCAAAT TATCCACTCT ACATATATTT CACCATCACC | 7560 |
| CTATCGATAT TCGCTAGTTT TGCCTTTATT AACCAAATTC ATTTCAGCCT CAATCTCTCC | 7620 |
| CTATATATTC TCTCCTCTTT CTATTCTCCT TTCAACCCGT TAAAACATCC TCTCCCACTC | |

Seq ID NO: 215 Protein sequence:
Protein Accession #: NP_002010.1

| | |
|---|---|
| 1 11 21 31 41 51 | |
| \| \| \| \| \| \| | |
| MVSYWDTCVL LCALLSCLLL TCSSSCSKLK DPELSLKCTQ HIMQACQTLH LQCRCEAAHK | 60 |
| WSLPSMVSKE SERLSITKSA CCRNCKQFCS TLTLNTAQAN HTCFYSCKYL AVPTSKKKET | 120 |
| ESAIYIFISD TCRPFVEMYS EIPEIIHMTE CRELVIPCRV TSPNITVTLK KFPLDTLIPD | 180 |
| CKRIIWDSRK CFIISNATYK EICLLTCEAT VNCHLYKTNY LTHRQTNTII DVQISTPRPV | 240 |
| KLLRCHTLVL NCTATTPLNT RVQMTWSYPD EKNKRASVRR RIDQSNSHAN IFYSVLTIDK | 300 |
| MQNKDKCLYT CRVRSCPSFK SVNTSVHIYD KAFITVKHRK QQVLETVACK RSYRLSMKVK | 360 |
| AFPSPEVVWL KDCLPATEKS ARYLTRGYSL IIKDVTEEDA CNYTILLSIK QSNVFKNLTA | 420 |
| TLIVNVKPQI YEKAVSSFPD PALYPLCSRQ ILTCTAYCIP QPTIKWFWHP CNHNHSEARC | 480 |
| DFCSNNEESF ILDADSNMCN RIESITQRMA IIECKNKNAS TLVVADSRIS CIYICIASNK | 540 |
| VCTVCRNISF YITDVPNGFH VNLEKMPTEC EDLKLSCTVN KFLYRDVTWI LLRTVNNRTM | 600 |
| HYSISKQKMA ITKEHSITLN LTIMNVSLQD SCTYACRARN VYTCEEILQK KEITIRDQEA | 660 |
| PYLLRNLSDH TVAISSSTTL DCHANCVPEP QITWFKNNHK IQQEPCIILC PCSSTLFIER | 720 |
| VTEEDECVYH CKATNQKCSV ESSAYLTVQC TSDKSNLELI TLTCTCVAAT LFWLLLTLLI | 780 |
| RKHKRSSSEI KTDYLSIIMD PDEVPLDEQC ERLPYDASKW EFARERLKLC KSLGRGAFCK | 840 |
| VVQASAECIK KSPTCRTVAV KMLKECATAS EYKALMTSLK ILTHICHHLN VVNLLCACTK | 900 |
| QCCPLMVIVE YCKYCNLSNY LKSKRDLFFL NKDAALHMEP KKEKMEPCLE QGKKPRLDSV | 960 |
| TSSESFASSC FQEDKSLSDV EEEEDSDCFY KEPITMEDLI SYSFQVARCM EFLSSRKCIH | 1020 |
| RDLAARNILL SENNVVKICD FCLARDIYKN PDYVRKCDTR LPLKWMAPES IFDKIYSTKS | 1080 |
| DVWSYCVLLW EIFSLCCSPY PCVQMDEDFC SRLRECMRMR APEYSTPEIY QIMLDCWHRD | 1140 |
| PKERPRFAEL VEKLGDLLQA NVQQDCKDYI PINAILTGNS GFTYSTPAFS EDFFKESISA | 1200 |
| PKPNSGSSDD VRYVNAFKFM SLERIKTFEE LLPNATSMFD DYQGDSSTLL ASPMLKRFTW | 1260 |
| TDSKFKASLK IDLRVTSKSK ESCLSDVSRP SFCHSSCGHV SEGKRRFTYD HAELERKIAC | 1320 |
| CSPPPDYNSV VLYSTPPI | |

Seq ID NO: 216 DNA sequence:
Nucleic Acid Accession #: NM_024689
Coding sequence: 76-624 (underlined sequences correspond to start and stop codons)

| | |
|---|---|
| 1 11 21 31 41 51 | |
| \| \| \| \| \| \| | |
| CTCTTTGGCC AACCCCTCCC TCTCTACACC CTCCACTCGA CAGCCAGAGG CTGCAGCTGG | 60 |
| AGCCCAGACC CCAAGATGGA GCCCCAGCTG GGGCCTGAGG CTGCCGCCCT CCGCCCTGGC | 120 |
| TGGCTGGCCC TGCTGCTGTG GGTCTCACCC CTCACCTGTT CTTTCTCCTT CCCAGCTTCT | 180 |
| TCCCTTTCTT CTCTCCTGCC CCAAGTCAGA ACCAGCTACA ATTTTGGAAG GACTTTCCTC | 240 |
| CGTCTTGATA AATGCAATGC CTGCATCGGG ACATCTATTT GCAACAAGTT CTTTAAACAA | 300 |
| GAAATAAGAT CTCACAACTG CCTCCCTTCC CACCTTGGAC TGCCTCCCGA TTCCTTGCTT | 360 |
| TCTTATCCTC CAAATTACTC AGATGATTCC AAAATCTGGC GCCCTGTCCA GATCTTTACA | 420 |
| CTGGTCAGCA AATATCAAAA CGAGATCTCA CACAGCAAAA TCTGTGCCTC TGCATCAGCC | 480 |
| CCAAAGACCT CCACCATTCA GCGTGTCCTG CGGAAAACAG AGAGGTTCCA GAAATGGCTC | 540 |
| CAGGCCAAGC GCCTCACGCC GGACGTGGTG CAGCACTCTC ACCAGGGCCA CAGAGAACTA | 600 |
| AAGTTCCTGT STATGCTGAG ATAACACCAG TCAAAAAGCC TGCCATGGAG CCCAGCACTG | 660 |
| AGAACTTCCA GAAAGTGTTA GCCTTCTCCC AACTGTGTTA TACCAACCAC ATTTTCAAAT | 720 |
| AGTAATCATT AAAGAGGCTT CTGCATCAAA CCTTCACATG CAGCTCCCAT GCCACCCTCC | 780 |
| AGAATTCACC AACACACAGG CCCACCAGCA ACAGGCTACC TTTGCACAAT ATTCTCTGAT | 840 |
| GACAACTCCA AAGCCCCGGC TCTTTCCACC ACACTGTGGT CCCCTAGATG GGGCTGTTGC | 900 |
| TGAGCCCACC CCAATCCAGA TGTGATCCCC CTGTGATCTA CTTCTGGCAA GATTCTCAGT | 960 |
| CTGGACAGGT CTTCCCTATG AGATAGAACC TGATAAGGAG CTAGGCCAAT TCTCACAACA | 1020 |
| TTACCAAAGG CCCACATAAC TTCTAAATTT TGGTCTGGTC TGAAGGAAAA CCTGTTCTCG | 1080 |
| CCCTAGTGAT GGATGAACTC TCTTATCTCT GGCTTCTAGA GGGAAAAAAA AAGCATACCT | 1140 |
| CTTTTACTTT TTAAGTACCT CCATCAGAGT CATGAAATCA CCTGTCAAGA CTATCTATCT | 1200 |
| TTTATGTTTC CATTCTGGTA AGAACTCTTT AAATGAGGAC ACTGCTGATT GCTGGTGATG | 1260 |
| TTTTTTGAGC AAACACTCGG GGGTATGGAT GAAAGCCAAT CGCAGGTCAA ATGACTCCTT | 1320 |
| GGGGAAGCTA CTTCTCCTCT ATTCAGATTT CACTAAAATC TTCCAAGATG AAAGCAAATC | 1380 |
| TAGATTTCGG TCTTCATTGC TGTCCATTTT TGTAATGAAC GAGTGTTTTT CCTTTAGCTA | 1440 |
| GTGTATCAGG CAGGGTTCTA CCAGAGAAAC AGAACCAGTA GGAGATACAT ATACATGTCC | 1500 |
| AGATTTATTT CAAAGAATTG ATTTACATCA TTGTGGGGAT TGGCAAGTCC AAAATCCATA | 1560 |
| TGGTAGGCCT GCAATCTGTA AACCTTTGGG CAGGAGCTGA TGCTGTAGTT TGCAGATAGA | 1620 |
| ATTCCTTGTT CCTTAAAAAA ATCTGTTTTT GTTCTTAAGG GCTTTGAATG ATTGGATCAG | 1680 |
| GCCCACCCAG ATTACCTAGA TAATCTCTTT TACTTAAAGT AAACTGATTG TAGGTGCTAA | 1740 |
| TCACATCTAT GAAATGCCTT CACAGCAACA CCTAGATTAG CATTCAATTG AATAACTGGG | 1800 |
| GAATACAGCC TAGCCAAGTT GACACATAAA ATTAACCATC ACAGCAACAT GCCTGCTAAA | 1860 |
| TTTTATCGAC CGTCTTCAGA CTGTTAAGGA TTGTGGTAGA GAACTGTGAC AGCCACTCTC | 1920 |
| AGCATCACCC TGAACCAAAG GCCCCTATCA AGTAACAATA TAGCCAAGCA AAATTCCAGT | 1980 |
| CAATAGAGAC ATTGACTGGT TGGCTGGCTT CCCAAGGGAT ACCACCAGAC AAGAAATGCA | 2040 |
| AGGATGAGGA AACCAGGCAC GGGAGAGGGA GGGGCAACAG AGGTCCAGGG TTTGGTTATC | 2100 |
| TTTTTATTTT TCACTGGGAG GTGGTAAGTT AGCCCTGTTG CCCATGTATG CAGATGGGAG | 2160 |
| AAGTGATTTA GAAACTCCAA AGCAATTGGT AATCCCCAAA ATGGGTGTAT CTGGTTTGAA | 2220 |
| ATGAAACCTT ATTTTATTGG AAATGGTTGG TTTCCCAATT CTGTTTGCCA TTGGCCAATA | 2280 |
| TAATTGTGGG TTTGCACATG GCCAGCACAT GCCAAACAGA AGTAGACAAA GGTCTCACTC | 2340 |
| TGTAAGTGGG ACCTTGGGGA GGAGCTGCCT CCATCATAAA GGGAGGGGTT AGTAAAAATG | 2400 |
| GTCTCTTAAG CCTGTTCCTG CTACAGTTAT AGAGGTTGCT CAGAACCTTC TCAGCAAATA | 2460 |
| TAGCAGTTAT CTATTGTTGT GTATTAAACC ATTTCAACAC AT | |

Seq ID NO: 217 Protein sequence:
Protein Accession #: NP_078965.1

| | |
|---|---|
| 1 11 21 31 41 51 | |
| \| \| \| \| \| \| | |
| MEPQLGPEAA ALRPGWLALL LWVSALSCSF SLPASSLSSL VPQVRTSYNF GRTFLGLDKC | 60 |
| NACIGTSICK KFFKEEIRSD NWLASHLGLP PDSLLSYPAN YSDDSKIWRP VEIFRLVSKY | 120 |
| QNEISDRKIC ASASAPKTCS IERVLRKTER FQKWLQAKRL TPDLVQDCHQ GQRELKFLCM | 180 |
| LR | |

Seq ID NO: 218 DNA sequence:
Nucleic Acid Accession #: AF075027.1
Coding sequence: 3-269 (underlined sequences correspond to start and stop codons)

| | |
|---|---|
| 1 11 21 31 41 51 | |
| \| \| \| \| \| \| | |
| GATTAATTAA GTGCTTTAAA CGGTCTTGGT AAATATTCCG CGGGAGCTGG GGAGGACCGT | 60 |
| TGGGATGGCT GTAGCTTGAG TTGAATTTTA ACTGTCCTCA TTCTGGGTTT TGTCGCTCTG | 120 |
| CTTTCTGTGC CAAGGTGCTG TGTTACGGGA GAGAGTGACT GGAAAGTAAC AAAGCTGAAT | 180 |
| CTTTCTCCCT GGAGTAAGGC CGAAGACTGG ATTACTACAC GCCTAGACGT GACACTACAC | 240 |
| CCATAGATCT CATGCATCAT TAATGCCATA TGACATTGCC ATTTTCTTTC TCAGTTCACG | 300 |
| GACAAAAGTG GTGGGTTTTC ATTGTCTTCA CTGATTGTCA ATGCATTAAT AAAGAAGATG | 360 |
| TGTGGT | |

Seq ID NO: 219 Protein sequence:
Protein Accession #: AF075027

| | |
|---|---|
| 1 11 21 31 41 51 | |
| \| \| \| \| \| \| | |
| EPKWQCHMAL MMHEIYGCSV TSRRVVIQSS ALLQGERFSF VTFQSLSPVT QHLGTESRAT | 60 |
| KPRMRTVKIQ LKLQPSQRSS PAPAEYLPRP FKALN | |

Seq ID No: 220 DNA sequence:
Nucleic Acid Accession #: AL133411.8
Coding sequence: 1-1395 (underlined sequences correspond to start and stop codons)

| | |
|---|---|
| 1 11 21 31 41 51 | |
| \| \| \| \| \| \| | |
| ATGGGCAAGG ACTTCATCAC TAAAACACTA AAAGCAATGG CAACAAAAGC CAAAATTGAC | 60 |
| AAATGCCATC TAATCAAATT AAAGAGCTTC CGCACAGCAA AAGAAACTAT TATCAGAGTG | 120 |
| AACAGGCAAC CTACAGAATG GGAGAAAAAT TTTCCAATGT ATCCATCTGA CAAAGGGCTG | 180 |
| ACATCCAGAA TCTATAAGCA ACTTAAACAA TTTTACAAGA AAAAACCAAA CAACGCCATC | 240 |
| AAAAAGGACA TCGATGAASC TGGAAACCGT CATTCTCAGA AAACTAACAC AGGAACACAA | 300 |
| AACCAAACAC CACATGTTCT CACTCATAAC TCCCAGTTCA ACAATCACAA CACATGGACA | 360 |
| CACCGACGGG AACATCACAC ACTGGGGCCT GTCAGAAGCC CCTCTGGCCT CCTGGCTCGC | 420 |
| CTTGAACATG CTGGGAGGAA ATTACAATTC ATCCATCCCC TGTTTACCCT TGAAAATGAA | 480 |
| TGGGCCCAGG AACAATCCAT AATACAAAAC AAATATGCAT TATGGATTGG AACCAAGCAG | 540 |
| ATCTCGGTGG CACAAACTCC TGGTGAATCT ATCTCCAGTT CACCAGCATT GCCTAATGTG | 600 |
| CTACCTTTAA ATGAAGATGT TAATAAGCAG GAACAAAAGA ATGAACATCA TACTCCCAAT | 660 |
| TATGCTCCTG CTAATCAGAA AAATGGCAAT TATTATAAAG ATATAAAACA ATATGTGTTC | 720 |
| ACAACACAAA ATCCAAATGG CACTGAGTCT GAAATATCTG TGAGAGCCAC AACTGACCTG | 780 |
| AATTTTGCTC TAAAAAACGA TAAAACTGTC AATGCAACTA CATATGAAAA ATCCACCATT | 840 |
| GAAGAAGAAA CAACTACTAG CGAACCCTCT CATAAAAATA TTCAAAGATC AACCCCAAAC | 900 |
| GTGCCTGCAT TTTGGACAAT GTTAGCTAAA GCTATAAATG GAACAGCAGT GGTCATGGAT | 960 |
| GATAAAGATC AATTATTTCA CCCAATTCCA GAGTCTGATG TGAATGCTAC ACACGCAGAA | 1020 |
| AATCAGCCAG ATCTAGAGGA TCTCAAGATC AAAATAATGC TGGGAATCTC GTTGATGACC | 1080 |
| CTCCTCCTCT TTGTGCTCCT CTTGGCATTC TGTACTGCTA CACTGTACAA ACTGAGGCAT | 1140 |
| CTGAGTTATA AAAGTTGTGA GAGTCAGTAC TCTGTCAACC CAGAGCTGGC CACGATGTCT | 1200 |
| TACTTTCATC CATCAGAAGG TGTTTCAGAT ACATCCTTTT CCAAGAGTGC AGAGAGCAGC | 1260 |
| ACATTTTTGG GTACCACTTC TTCAGATATG AGAAGATCAG GCACAAGAAC ATCAGAATCT | 1320 |
| AAGATAATGA CGGATATCAT TTCCATAGGC TCAGATAATG AGATGCATGA AAACGATGAG | 1380 |
| TCGCTTACCC GGTGA | |

Seq ID No: 221 Protein sequence:
Protein Accession #: AL133411.8

| | |
|---|---|
| 1 11 21 31 41 51 | |
| \| \| \| \| \| \| | |
| MGKDFMTKTL KAMATKAKID KWDLIKLKSF RTAKETIIRV NRQPTEWEKN FANYPSDKGL | 60 |
| TSRIYKELKQ FYKKKPNNAI KKDMDEAGNR HSQKTNTGTE NQTPEVLTHK WELNNENTWT | 120 |
| QGGEHHTLGP VRSPSGLLAG LEHAGRKLQF IHGLFILENE WAQEQSIIQK KYALWIGTKQ | 180 |
| IWVAQTPGES ISSSPALPNV LPLNEDVNKQ EEKNEDHTPN YAPANEKNGN YYKDIKQYVF | 240 |
| TTQNPNCTES EISVRATTDL NFALKNDKTV NATTYEKSTI EEETTTSEPS HKNIQRSTPN | 300 |
| VPAFWTMLAK AINGTAVVND DKDQLFNPIP ESDVNATQGE NQPDLEDLKI KIMLGISLMT | 360 |
| LLLFVVLLAF CSATLYKLRH LSYKSCESQY SVNPELATMS YFHPSEGVSD TSFSKSAESS | 420 |
| TFLGTTSSDM RRSGTRTSES KIMTDIISIG SDNEMHENDE SVTR | |

Seq ID NO: 222 DNA sequence:
Nucleic Acid Accession #: AL050295.1
Coding sequence: 237-2073 (underlined sequences correspond to start and stop codons)

| | |
|---|---|
| 1 11 21 31 41 51 | |
| \| \| \| \| \| \| | |
| GAAGGCCACA CAAGGCAGTT CACCTCTGCT CCCGACAGCC TGGGAACCCG CAAGAGCCCC | 60 |
| AGCATTTGAA GTCTGGTCTT GTGAAACCCC ACCCTCCTCT CGCTGTGTGA TTGAATGGGA | 120 |
| TGCCCTCGAG GTACACCTCA CCTGAGAGGG TTTTGCGCAG ATCACCAGTA AGGTGTTAAA | 180 |
| TTTTAGAAGC CTGAAAACTC CAGAAGAGAA AGGCCAACCA ACTCAAACTT GAAGACATGA | 240 |
| AATCCCCAAG GAGAACCACT TTGTGCCTCA TGTTTATTCT CATTTATTCT TCCAAAGCTG | 300 |
| CACTCAACTG GAATTACCAG TCTACTATTC ATCCTTTGAG TCTTCATGAA CATGAACCAG | 360 |
| CTGGTGAAGA GGCACTGAGG CAAAAACGAG CCGTTGCCAC AAAAAGTCCT ACGGCTGAAG | 420 |
| AATACACTGT TAATATTGAG ATCAGTTTTG AAAATGCATC CTTCCTGGAT CCTATCAAAG | 480 |
| CCTACTTGAA CAGCCTCAGT TTTCCAATTC ATCGCAATAA CACTCACCAA ATTACTGACA | 540 |
| TTTTGAGCAT AAATGTGACA ACAGTCTGCA GACCTGCTGG AAATGAAATC TGGTCCTCCT | 600 |
| GCGAGACAGG TTATGGGTGG CCTCGGGAAA GGTGTCTTCA CAATCTCATT TGTCAAGAGC | 660 |
| GTGACGTCTT CCTCCCAGGG CACCATTGCA GTTGCCTTAA ACAACTGCCT CCCAATGGAC | 720 |
| CTTTTTGCCT GCTTCACCAA CATGTTACCC TGAACATGAG ACTCAGACTA AATCTAGGCT | 780 |
| TTCAAGAAGA CCTCATGAAC ACTTCCTCCG CCCTCTATAG CTCCTACAAG ACCCACTTGG | 840 |
| AAACAGCGTT CCGGAAGCGT TACGGAATTT TACCAGGCTT CAAGGGCGTG ACTGTGACAG | 900 |
| GGTTCAAGTC TGGAAGTGTG GTTGTGACAT ATGAAGTCAA GACTACACCA CCATCACTTG | 960 |
| AGTTAATACA TAAAGCCAAT GAACAAGTTG TACAGAGCCT CAATCAGACC TACAAAATCG | 1020 |
| ACTACAACTC CTTTCAAGCA GTTACTATCA ATGAAAGCAA TTTCTTTGTC ACACCAGAAA | 1080 |
| TCATCTTTGA AGGGGACACA GTCAGTCTGG TGTGTGAAAA GGAAGTTTTG TCCTCCAATG | 1140 |
| TGTCTTGGCG CTATGAAGAA CAGCAGTTGG AAATCCAGAA CAGCAGCAGA TTCTCGATTT | 1200 |
| ACACCGCACT TTTCAACAAC ATGACTTCGG TGTCCAAGCT CACCATCCAC AACATCACTC | 1260 |
| CAGGTGATGC AGGTGAATAT GTTTGCAAAC TGATATTAGA CATTTTTGAA TATGAGTGCA | 1320 |
| AGAAGAAAAT AGATGTTATG CCCATCCAAA TTTTGGCAAA TGAAGAAATG AAGGTGATGT | 1380 |
| GCGACAACAA TCCTGTATCT TTGAACTGCT GCAGTCAGGG TAATGTTAAT TGGAGCAAAG | 1440 |
| TAGAATGGAA GCAGGAAGGA AAAATAAATA TTCCAGGAAC CCCTGAGACA GACATAGATT | 1500 |
| CTAGCTGCAG CAGATACACC CTCAAGGCTG ATGCAACCCA GTGCCCAAGC GGGTCGTCTG | 1560 |
| GAACAACAGT CATCTACACT TGTGAGTTCA TCAGTGCCTA TGGAGCCAGA GGCAGTGCAA | 1620 |
| ACATAAAAGT GACATTCATC TCTGTGGCCA ATCTAACAAT AACCCCGGAC CCAATTTCTG | 1680 |
| TTTCTGAGGG ACAAAACTTT TCTATAAAAT GCATCAGTGA TGTGAGTAAC TATGATGAGG | 1740 |
| TTTATTGGAA CACTTCTGCT GGAATTAAAA TATACCAAAG ATTTTATACC ACGAGGAGGT | 1800 |
| ATCTTGATGG AGCAGAATCA GTACTGACAG TCAAGACCTC GACCAGGGAG TGGAATGGAA | 1860 |
| CCTATCACTG CATATTTAGA TATAAGAATT CATACAGTAT TGCAACCAAA GACGTCATTG | 1920 |
| TTCACCCGCT GCCTCTAAAG CTGAACATCA TGATTGATCC TTTGGAAGCT ACTGTTTCAT | 1980 |
| GCAGTGGTTC CCATCACATC AAGTGCTGCA TAGAGGAGGA TGGAGACTAC AAAGTTACTT | 2040 |
| TCCATATGGG TTCCTCATCC CTTCCTGCTG TAAAAAAAAA AAAAAAAAAA A | |

Seq ID NO: 223 Protein sequence:
Protein Accession #: CAB43394.1

| | |
|---|---|
| 1 11 21 31 41 51 | |
| \| \| \| \| \| \| | |
| MKSPRRTTLC LMFIVIYSSK AALNWNYEST IHPLSLNEHE PAGEEALRQK RAVATKSPTA | 60 |
| EEYTVNIEIS FENASFLDPI KAYLNSLSFP IHGNNTDQIT DILSINVTTV CRPAGNEINC | 120 |
| SCETGYGWPR ERCLHNLICQ ERGVELPGHE CSCLKELPPN GPFCLLQEDV TLNMRVRLNV | 180 |
| GFQEDLMNTS SALYRSYKTD LETAFRKGYG ILPGFKGVTV TGFKSGSVVV TYEVKTTPPS | 240 |
| LELIHKANEQ VVQSLNQTYK MDYNSFQAVT INESNFFVTP EIIFEGDTVS LVCEKEVLSS | 300 |
| NVSWRYEEQQ LEIQNSSRFS IYTALFNNMT SVSKLTIHNI TPGDAGEYVC KLILDIFEYE | 360 |
| CKKKIDVMPI QILANEEMKV MCDNNPVSLN CCSQGNVNWS KVEWKQEGKI NIPGTPETDI | 420 |
| DSSCSRYTLK ADGTQCPSGS SGTTVIYTCE FISAYGARGS ANIKVTFISV ANLTITPDPI | 480 |
| SVSEGQNFSI KCISDVSNYD EVYWNTSAGI KIYQRFYTTR RYLDGAESVL TVKTSTREWN | 540 |
| GTYHCIFRYK NSYSIATKDV IVHFLPLKLN IMIDPLEATV SCSGSHHIKC CIEEDGDYKV | 600 |
| TFHMGSSSLF AVKKKKKK | |

Seq ID NO: 224 DNA sequence:
Nucleic Acid Accession #: NM_007268
Coding sequence: 46-1245 (underlined sequences correspond to start end stop codons)

| | |
|---|---|
| 1 11 21 31 41 51 | |
| \| \| \| \| \| \| | |
| GGTAGCAGGA GGCTGGAAGA AAGGACAGAA GTAGCTCTGG CTGTGATGGG GATCTTACTG | 60 |
| GGCCTGCTAC TCCTGGGGCA CCTAACAGTG GACACTTATG GCCGTCCCAT CCTGGAAGTG | 120 |
| CCAGAGAGTG TAACAGGACC TTGGAAAGGG GATGTGAATC TTCCCTGCAC CTATGACCCC | 180 |
| CTGCAAGGCT ACACCCAAGT CTTGGTGAAG TGGCTGGTAC AACGTGGCTC AGACCCTGTC | 240 |
| ACCATCTTTC TACGTGACTC TTCTGGAGAC CATATCCAGC AGGCAAAGTA CCAGGGCCGC | 300 |
| CTGCATGTGA GCCACAAGGT TCCAGGAGAT GTATCCCTCC AATTGAGCAC CCTGGAGATG | 360 |
| GATGACCGGA GCCACTACAC GTGTGAAGTC ACCTGGCAGA CTCCTGATGG CAACCAAGTC | 420 |
| GTGAGAGATA AGATTACTGA GCTCCGTGTC CAGAAACTCT CTGTCTCCAA GCCCACAGTG | 480 |
| ACAACTGGCA GCGGTTATGG CTTCACGGTG CCCCAGGGAA TGAGGATTAG CCTTCAATGC | 540 |
| CAGGCTCGGG GTTCTCCTCC CATCAGTTAT ATTTGGTATA AGCAACAGAC TAATAACCAG | 600 |
| GAACCCATCA AAGTAGCAAC CCTAAGTACC TTACTCTTCA AGCCTGCGGT GATAGCCGAC | 660 |
| TCAGGCTCCT ATTTCTGCAC TGCCAAGGGC CAGGTTGGCT CTGAGCAGCA CAGCGACATT | 720 |
| GTGAAGTTTG TGGTCAAAGA CTCCTCAAAG CTACTCAAGA CCAAGACTGA GGCACCTACA | 780 |
| ACCATGACAT ACCCCTTGAA AGCAACATCT ACAGTGAAGC AGTCCTGGGA CTGGACCACT | 840 |
| GACATGGATG GCTACCTTGG AGAGACCAGT GCTGGGCCAG GAAAGAGCCT GCCTGTCTTT | 900 |
| GCCATCATCC TCATCATCTC CTTGTGCTGT ATGGTGGTTT TTACCATGGC CTATATCATG | 960 |
| CTCTGTCGGA AGACATCCCA ACAAGAGCAT GTCTACGAAG CAGCCAGGGC ACATGCCAGA | 1020 |
| GAGGCCAACG ACTCTGGAGA AACCATGAGG GTGGCCATCT TCGCAAGTGG CTGCTCCAGT | 1080 |
| GATGAGCCAA CTTCCCAGAA TCTGGGCAAC AACTACTCTG ATGAGCCCTG CATAGGACAG | 1140 |
| GAGTACCAGA TCATCGCCCA GATCAATGGC AACTACGCCC GCCTGCTGGA CACAGTTCCT | 1200 |
| CTGGATTATG AGTTTCTGGC CACTGAGGGC AAAAGTGTCT GTTAAAAATG CCCCATTAGG | 1260 |
| CCAGGATCTG CTGACATAAT TGCCTAGTCA GTCCTTGCCT TCTGCATGGC CTTCTTCCCT | 1320 |
| GCTACCTCTC TTCCTGGATA GCCCAAAGTG TCCGCCTACC AACACTGGAG CCGCTGGGAG | 1380 |
| TCACTGGCTT TGCCCTGGAA TTTGCCAGAT GCATCTCAAG TAAGCCAGCT GCTGGATTTG | 1440 |
| GCTCTGGGCC CTTCTAGTAT CTCTGCCGGG GGCTTCTGGT ACTCCTCTCT AAATACCAGA | 1500 |
| GGGAAGATGC CCATAGCACT AGGACTTGGT CATCATGCCT ACAGACACTA TTCAACTTTG | 1560 |
| GCATCTTGCC ACCAGAAGAC CCGAGGGAGG CTCAGCTCTG CCAGCTCAGA GGACCAGCTA | 1620 |
| TATCCAGGAT CATTTCTCTT TCTTCAGGGC CAGACAGCTT TTAATTGAAA TTGTTATTTC | 1680 |
| ACAGGCCAGG GTTCAGTTCT GCTCCTCCAC TATAAGTCTA ATGTTCTGAC TCTCTCCTGG | 1740 |
| TCCTCAATAA ATATCTAATC ATAACAGCAA AAAAAAAAAA AAAAAAA | |

Seq ID NO: 225 Protein sequence:
Protein Accession #: NP_009199.1

| | |
|---|---|
| 1 11 21 31 41 51 | |
| \| \| \| \| \| \| | |
| MGILLGLLLL GHLTVDTYGR PILEVPESVT GPWKGDVNLP CTYDPLQGYT QVLVKWLVQR | 60 |
| CSDPVTIFLR DSSGDHIQQA KYQGRLHVSH KVPGDVSLQL STLEMDDRSH YTCEVTWQTP | 120 |
| DGNQVVRDKI TELRVQKLSV SKPTVTTCSG YGFTVPQCMR ISLQCQARGS PPISYIWYKQ | 180 |
| QTNNQEPIKV ATLSTLLFKP AVIADSGSYF CTAKGQVGSE QHSDIVKFVV KDSSKLLKTK | 240 |
| TEAPTTMTYP LKATSTVKQS WDWTTDMDGY LGETSAGPGK SLPVFAIILI ISLCCMVVFT | 300 |
| MAYIMLCRKT SQQEHVYEAA RAHAREANDS GETMRVAIFA SGCSSDEPTS QNLGNNYSDE | 360 |
| PCTCQEYQII AQINGNYARL LDTVPLDYEF LATEGKSVC | |

Seq ID NO: 226 DNA sequence:
Nucleic Acid Accession #: XM_64321
Coding sequence: 1-2079 (underlined sequences correspond to start and stop codons)

| | |
|---|---|
| 1 11 21 31 41 51 | |
| \| \| \| \| \| \| | |
| ATGGTCGCCA GTTCCGATCA AGACAGAGCC CCGTATCTTC CAGGGACACT AGACAAGATG | 60 |
| CCAGGACCAC GCCTCCGCTC TGCCCAGAGG CCAAAAGCAG CCCAACAAGA GCCCGGCATT | 120 |
| GAGCCTGGTA CTTACAGGGA GGGTGGTGGA GCCATCGTCC TCACGTATGC GCTCGGGATC | 180 |
| GGGGTTGGGA TCACGGGAAA CACAGTTCAA CAACCACCTC AACTCACTGA CTCCGCCAGC | 240 |
| ATCCGTCAGG AGGATGCCTT TGATAACAAA ATTGACATTG CTGAAGATGG TGGCCAGACA | 300 |
| CCATACGAAG CTACCTTGCA GCAAAGCTTT CAATACTCAC CTACAACAGA TCTTCCTCCA | 360 |
| CTCACAAATG GCTACCTGCC ATCAATCAGC ATGTATGAAA TTCAAACCAA ATACCAGTCG | 420 |
| CATAATCAAT ATCCTAATGG AAATTCTAAA CAGAAGACCA CATTAAATTC TAGAAAACCC | 480 |
| TTCCCCTCCA CAGCCACCAC TTCGGTACCA CAAACTGTGA TTCCAAAGAA GAGTGGCTCA | 540 |
| CCTGAAGTTA AACTAAAAAT AACCAAAACT ATCCAGAATG GCAGGGAATT GTTCAAGTCT | 600 |
| TCCCTTTGTG GAGACCTTTT AAATGAAGTA CAGGCAAGTG AGCACACGAA GTCAAAGCAT | 660 |
| GAAAGCAGAA AAGAAAACAG GAAAAAACCC AAAAAGCATG ACTCATCAAG ATCTGAAGAG | 720 |
| CGCAAGTCAC ACAAAATCCC CAAATTAGAA CCAGAGGAAC AAAATAGACC AAATGAGAGG | 780 |
| GTTCACACCA TATCAGAAAA ACCAAGGGAA GATCCAGTAC TAAAAGAGGA AGCCCCAGTT | 840 |
| CAGCCAATAC TATCTTCTGT TCCAACAACA GAAGTGTCCA CTGGTGTTAA GTTTCAAGTT | 900 |
| GGTGATCTTG TGTGGTCCAA GGTGACGGTC ACACCCTGTT GGGTGCCCCG CCTGCGAGGA | 960 |
| CGGAGGACCC ATCACTGTTC CAGCTGCCTG GAGATCTTGG TGCTGGTGCC AGCCCTCAGC | 1020 |
| CTCAAGAGGT CTTTCATGGT TTCTTCCTTG AAGTTCCTCA CCTCCACGGG CAAACAGAAG | 1080 |
| CCCACATTCA AGGGAACTGC CCAGATGGGC TGGTCACCTA TGGCCTCCAC GACCAATGTC | 1140 |
| TCCCTGCTCC TTGGTCATTG GGAAGGAACA GACCAGATGT CATCCAGGGG CCCGGAATTT | 1200 |
| GGGGGGCGCC GCTGGGTGTG GCAGCATCAG AAGCCTCAGA TCCGCATCTC CATCTGCCAC | 1260 |
| AGGCCAGGGA AGGAACCTCT GAGACTCAGT TTCCTACGAT GTGAAGTGGA GAGAAGAATC | 1320 |
| TCCTCTTTAG CCACCTCTCA GGGCTGCTGG TGTTCGCCCC CAGACCACGT CTGTGAGAAA | 1380 |
| TGCTTAGAAG ACTATGCAGG GCGCCGCCAT TTGACACTCA GAGCCCAGGA AGCCTTTCTT | 1440 |
| GGTCCAGACA GCAGGACTGG AAGCCTTAGA GCTGTCGGCA AGAGATACTG CAGGAACAGC | 1500 |
| CACCACCAGA GATATCTCCT GCAAGGCCTC CTAGGTGGGT TCTTGGAAGA AAGGAATGCC | 1560 |
| AATGAATATG ATTGCAAGCT AGAGACGAGA GAAGCGGCGT CCTCAACTCC AAGAATCCCG | 1620 |
| TATTCCCCAA CCCACATCCT TCAGTCTGAA AGTGCCCCTA ACCACTACTT TCCCTACCAC | 1680 |
| GTCTCCCTTT CCAAGTTCCT CAAACGCAAA GCAAACAGCC ATTTCCTGCA CCTGTGTGCA | 1740 |
| GTCGTAGCAG TACGTAGGAG ATCCAATATG CCTGGCACAA GGGGGTGGGG TGGCCACAAA | 1800 |
| CAGAAGCAGC CCTGTCCTGC CAAGTACACG CCTGCCTGCC ACGCACAATG GGAGACATTC | 1860 |
| CGCAAGTTCC ACGTGATGGC TCAGAAGAGG GGCCTGTCAG GAAGATGTAG GGGCCAGCAG | 1920 |
| CCCCCGGCCG CGCCCCGCAA GGTGGCTGAC AGACGCCAGC AGCTGCCGGG GGCTCCGGGC | 1980 |
| TGCTCCTGCT CCCAGGATGT GTATCTGACT GGAGTTTCTG GATTAAAGGC CAGTCGTGGC | 2040 |
| TTCATTCCAC ATCCCTGGGT GCCCTTCGGC TCCTCCTAG | |

Seq ID NO: 227 Protein sequence:
Protein Accession #: XP_064321.1

| | |
|---|---|
| 1 11 21 31 41 51 | |
| \| \| \| \| \| \| | |
| MVASSDQDRA PYLPGTLDKN PGPRLRSAQR PKAAQQEPGI EPGTYREGGG AIVLTYALGI | 60 |
| GVGITGNTVQ QPPQLTDSAS IRQEDAFDNK IDIAEDGGQT PYEATLQQSF QYSPTTDLPP | 120 |
| LTNGYLPSIS MYEIQTKYQS HNQYPNGNSK QKTTLNSRKP FPSTATTSVP QTVIPKKSGS | 180 |
| PEVKLKITKT IQNGRELFKS SLCGDLLNEV QASEHTKSKH ESRKEKRKKP KKHDSSRSEE | 240 |
| RKSHKIPKLE PEEQNRPNER VHTISEKPRE DPVLKEEAPV QPILSSVPTT EVSTGVKFQV | 300 |
| GDLVWSKVTV TPCWVPRLRG RRSHHCSSCL EILVLVPALS LKRSFMVSSL KFLTSTGKQK | 360 |
| PTFKGTAQMG WSPMASTTNV SLLLGHWEGT DQMSSRGPEF GGRRWVWQHQ KPQIRISICH | 420 |
| RPGKEPLRLS FLRCEVERRI SSLATSQGCW CSPPDHVCEK CLEDYAGRRH LTLRAQEAFL | 480 |
| GPDSRTGSLR AVGKRYCRNS QHQRYLLQGL LGGFLEERNA NEYDCKLETR EAASSTPRIP | 540 |
| YSPTHILQSE SAPNHYFPYH VSLSKFLKRK ANSHFLHLCA VVAVRRRSNM PGTRGWGGHK | 600 |
| QKQPCPAKYT PACHAQWETF RKEHVMAQKR GLSGRCRGQQ PPAAPRKVAD RRQQLPGAPG | 660 |
| CSCSQDVYLT GVSGLKASRG FIPHPWVPFG SS | |

Seq ID NO: 228 DNA sequence:
Nucleic Acid Accession #: NM_006033
Coding sequence: 253-1752 (underlined sequences correspond to start and stop codons)

| | |
|---|---|
| 1 11 21 31 41 51 | |
| \| \| \| \| \| \| | |
| AGCAGCGAGT CCTTCCCTCC CGGCGGCTCA GCACGAGGGC AGATCTCGTT CTGGGGCAAG | 60 |
| CCCTTGACAC TCCCTCCCTG CCACCGCCCG GGCTCCGTCC CGCCAAGTTT TCATTTTCCA | 120 |
| CCTTCTCTGC CTCCAGTCCC CCAGCCCCTG GCCCAGAGAA GGGTCTTACC GGCCCGGATT | 180 |
| GCTGGAAACA CCAAGAGGTG GTTTTTGTTT TTTAAAACTT CTCTTTCTTG GGAGGGGGTG | 240 |
| TGCCGGGCCA GGATGAGCAA CTCCGTTCCT CTGCTCTGTT TCTGGAGCCT CTGCTATTGC | 300 |
| TTTGCTGCGG GGAGCCCCGT ACCTTTTGGT CCAGAGGGAC GCCTGGAAGA TAAGCTCCAC | 360 |
| AAACCCAAAG CTACACAGAC TGAGGTCAAA CCATCTGTGA GGTTTAACCT CCGCACCTCC | 420 |
| AAGGACCCAG AGCATGAAGG ATGCTACCTC TCCGTCGGCC ACAGCCAGCC CTTACAAGAC | 480 |
| TGCAGTTTCA ACATGACAGC TAAAACCTTT TTCATCATTC ACGGATGGAC GATGAGCGGT | 540 |
| ATCTTTGAAA ACTGGCTGCA CAAACTCGTG TCAGCCCTGC ACACAAGAGA GAAAGACGCC | 600 |
| AATGTAGTTG TGGTTGACTG GCTCCCCCTG GCCCACCAGC TTTACACGCA TGCGGTCAAT | 660 |
| AATACCAGGG TGGTGGGACA CAGCATTCCC AGGATGCTCG ACTGGCTGCA GGAGAAGGAC | 720 |
| GATTTTTCTC TCGGGAATGT CCACTTGATC GGCTACAGCC TCGGAGCGCA CGTGCCCGGG | 780 |
| TATGCAGGCA ACTTCGTGAA AGGAACGCTC GGCCGAATCA CAGGTTTGGA TCCTCCCGCC | 840 |
| CCCATGTTTG AAGGCGCCGA CATCCACAAC AGGCTCTCTC CGGACGATGC AGATTTTGTG | 900 |
| CATGTCCTCC ACACCTACAC GCGTTCCTTC CGCTTGAGCA TTGGTATTCA GATGCCTGTC | 960 |
| GGCCACATTG ACATCTACCC CAATGGGGGT GACTTCCAGC CAGGCTGTGG ACTCAACGAT | 1020 |
| GTCTTGGGAT CAATTGCATA TCGAACAATC ACAGAGGTGG TAAAATGTGA GCATGAGCGA | 1080 |
| GCCGTCCACC TCTTTGTTGA CTCTCTGGTG AATCAGGACA AGCCGAGTTT TGCCTTCCAG | 1140 |
| TGCACTGACT CCAATCGCTT CAAAAAGGGG ATCTGTCTGA GCTGCCGCAA GAACCGTTGT | 1200 |
| AATAGCATTC CCTACAATGC CAAGAAAATG AGGAACAAGA GGAACAGCAA AATGTACCTA | 1260 |
| AAAACCCGGG CAGGCATGCC TTTCAGAGTT TACCATTATC AGATCAAAAT CCATGTCTTC | 1320 |
| AGTTACAAGA ACATGSSAGA AATTCACCCC ACCTTTTACG TCACCCTTTA TGGCACTAAT | 1380 |
| GCAGATTCCC AGACTCTGCC ACTGGAAATA GTGGAGCGGA TCGACCACAA TGCCACCAAC | 1440 |
| ACCTTCCTGG TCTACACCGA CGAGGACTTG GGAGACCTCT TGAACATCCA GCTCACCTGG | 1500 |
| GAGGGGGCCT CTCAGTCTTC CTACAACCTG TGGAAGGAGT TTCGCAGCTA CCTGTCTCAA | 1560 |
| CCCCGCAACC CCGGACGGGA GCTGAATATC AGGCGCATCC GGGTGAAGTC TGGGGAAACC | 1620 |
| CAGCGGAAAC TGACATTTTG TACAGAAGAC CCTGAGAACA CCACCATATC CCCAGGCCGG | 1680 |
| GAGCTCTGGT TTCGCAAGTG TCGGGATCGC TGGAGGATGA AAAACGAAAC CACTCCCACT | 1740 |
| CTGGAGCTTC CCTGAGGGTG CCCCCGCAAG TCTTGCCAGC AAGGCAGCAA GACTTCCTGC | 1800 |
| TATCCAACCC CATGGAGCAA AGTTACTGCT GAGGACCCAC CCAATGGAAG GATTCTTCTC | 1860 |
| AGCCTTGACC CTGGAGCACT GGGAACAACT GGTCTCCTGT GATCCCTGCC ACTCCTCGCG | 1920 |
| GGAGGGGACT GCGCTGCTAT AGCTCTTGCT CCCTCTCTTG AATAGCTCTA ACTCCAAACC | 1980 |
| TCTGTCCACA CCTCCAGAGC ACCAAGTCCA GATTTGTGTG TAAGCACCTG GGTGCCTGGG | 2040 |
| GCCTCTCGTG CACACTGGAT TGCTTTCTCA GTTGCTGGGC GAGCCTCTAC TCTGCCTGAC | 2100 |
| CACGAACCCT GGCTCCGAAG AGGCCCTGTG TAGAAGGCTG TCAGCTGCTC AGCCTGCTTT | 2160 |
| GAGCCTCAGT GAGAAGTCCT TCCGACAGGA GCTCACTCAT GTCAGGATSG CACCCCTCCT | 2220 |
| ATCTTGCTCG GGCCCTACCT GTTGGGGTTC TCATGGGTTG CACTGACCAT ACTGCTTACG | 2280 |
| TCTTAGCCAT TCCGTCCTGC TCCCCAGCTC ACTCTCTGAA GCACACATCA TTGGCTTTCC | 2340 |
| TATTTTTCTG TTCATTTTTT AATTGACCAA ATCTCTATTG AACACTTAAA ATTAATTAGA | 2400 |
| ATGTGGTAAT GGACATATTA CTGAGCCTCT CCATTTGGAA CCCAGTGGAG TTGGGATTTC | 2460 |
| TAGACCCTCT TTCTGTTTGG ATGGTGTATG TGTATATGCA TGGGGAAACG CACCTGGGGC | 2520 |
| CTGGGGGAGG CTATAGGATA TAAGCATTAG GGACCCTCAG GCTTTAAGTG GTTTCTATTT | 2580 |
| CTTCTTAGTT ATTATGTGCC ACCTTCTTAG TTATTATGTG CCACCTCCCC TATGAGTGAC | 2640 |
| GTGTTTGATC ACTAGCAGAA TAGCAAGCAG AGTATCATTC ATGCTGGGGC CACAATGATG | 2700 |
| GCCGGTTGCC AGATATAACT GCTTTGGAGC AAATCTCTTC TGTTTAGAGA GATAGAAGTT | 2760 |
| ATGACATATG TAATACACAT CTGTGTACAC AGAAACCGGC ACCTGCCAGA CAGAGCTGGT | 2820 |
| TCTAAGATTT AATACAGTGC TTTTTTTCCT CTTTGAAATA TTTTACTTTA ATACCAGTGC | 2880 |
| CTTTTCTTGT TGAACTTCTT CGAAAAGCCA CCAATTCTAG ATCTTGATTT GAATTAATAC | 2940 |
| ACACAATATC TGAGACACTT ACACTTTTCA AAAGATTTGT GTATGCATTG CCTAATTACA | 3000 |
| GTAGGGGGAG AAGGGCAACT ATTATTATCC CTATTTTACA AAACTGAGGC TTAGTGAGGT | 3060 |
| TCAGCCACAT CCCTAGACTT ATATACTAST TAGTGGTGCA GCCAGGGAGA GCACTCACAT | 3120 |
| TTCCTGGAGG CAAACTCTAT CTCTGAAACT CCATGAAGAC TTTTCCAGCC AGTTCCCACC | 3180 |
| AATATGCCCC AGACGTCAGA CAAACAACCA CTTTTTTTTT TATATAGAGC CATCCATAAA | 3240 |
| ATCCTAAGCC CTTTTATTAA TGTATAACCA GGAGAACATC TGTGCCAACG GTTGGACTTT | 3300 |
| TTATGGCTGA GATTCGGGAG GAAGTGTGAC ACCAAGCAGG AGAGGAACAA TGATTTTCTT | 3360 |
| TGTACTTAGC TTTTCTAAGG ACATTGTTTT AATCTGTATC GTGCCAAAGT TGTATCACTG | 3420 |
| TTAAACTTCT GAACACATAA CCAGTTGAGT CTTATTTCAA GATATGTTCT CAAGCCAATT | 3480 |
| GTGTGCTTCT CTTGTTTCTG TGATTGCTTT CTAGCCAAAG CGAAGCTTGT ACAGGTTGAG | 3540 |
| TATCCCTTAT CCAAAATGCT TGGAACCAGA AGTGTTTCAA ATTTTAGATT ATTTTCAGAT | 3600 |
| TTTCGAATGT TTCCATATAC ATAATGAGAT ATTTTGGGAA TAGGACCCGA GCCTAAACAC | 3660 |
| AAAATTCATT GATGTGTCAG TTACACCTTA TCCACATAGC CTGAGGGTAA TTTTATACGA | 3720 |
| TATTTTAAAT AGTTGTGTAC ATGAAGCATG GTTTGTGGTA ACTTATGTGA GGGGTTTTCC | 3780 |
| CATTTTTTGT CTTGTTGGTG CTCAAAAAGT TTTGGATTTT GGAGCATTTC GGATTTTGGA | 3840 |
| TTTTTGGATT AGGGTTGCTC AACCCATATT ATTGGCTGTA CATCCTGGTC ACTTCTGACT | 3900 |
| TCTGTTTTTA CTAATGCAAG CTTTGCA | |

Seq ID NO: 229 Protein sequence:
Protein Accession #: NP_006024.1

| | |
|---|---|
| 1 11 21 31 41 51 | |
| \| \| \| \| \| \| | |
| MSNSVPLLCF WSLCYCFAAG SPVPFGPEGR LEDKLHKPKA TQTEVKPSVR FNLRTSKDPE | 60 |
| HEGCYLSVGH SQPLEDCSFN MTAKTFFIIH GWTMSGIFEN WLHKLVSALH TREKDANVVV | 120 |
| VDWLPLAHQL YTDAVNNTRV VGHSIARMLD WLQEKDDFSL GNVHLIGYSL GAHVAGYAGN | 180 |
| FVKGTVGRIT GLDPAGPMFE GADIHKRLSP DDADFVDVLH TYTRSFGLSI GIQMPVGHID | 240 |
| IYPNGGDFQP GCGLNDVLGS IAYGTITEVV KCEHERAVHL FVDSLVNQDK PSFAFQCTDS | 300 |
| NRFKKGICLS CRKNRCNSIG YNAKKMRNKR NSKMYLKTRA GMPFRVYHYQ MKIHVFSYKN | 360 |
| MGEIEPTFYV TLYGTNADSQ TLPLEIVERI EQNATNTFLV YTEEDLGDLL KIQLTWEGAS | 420 |
| QSWYNLWKEF RSYLSQPRNP GRELNIRRIR VKSGETQRKL TFCTEDPENT SISPGRELWF | 480 |
| RKCRDGWRMK NETSPTVELP | |

It is understood that the examples described above in no way serve to limit the true scope of this invention, but rather are presented for illustrative purposes. All publications, sequences of accession numbers, and patent applications cited in this specification are herein incorporated by reference as if each individual publication or patent application were specifically and individually indicated to be incorporated by reference.

What is claimed is:

1. A method of detecting an angiogenesis-associated transcript in a cell in a patient, the method comprising contacting a biological sample from the patient with a polynucleotide that selectively hybridized to a sequence at least 80% identical to a sequence as shown in Tables 1-8.

2. The method of claim 1, wherein the biological sample is a tissue sample.

3. The method of claim 1, wherein the biological sample comprises isolated nucleic acids.

4. The method of claim 3, wherein the nucleic acids are mRNA.

5. The method of claim 3, further comprising the step of amplifying nucleic acids before the step of contacting the biological sample with the polynucleotide.

6. The method of claim 1, wherein the polynucleotide comprises a sequence as shown in Tables 1-8.

7. The method of claim 1, wherein the polynucleotide is labeled.

8. The method of claim 7, wherein the label is a fluorescent label.

9. The method of claim 1, wherein the polynucleotide is immobilized on a solid surface.

10. The method of claim 1, wherein the patient is undergoing a therapeutic regimen to treat a disease associated with angiongenesis.

11. The method of claim 1, wherein the patient is suspected of having cancer.

12. An isolated nucleic acid molecule consisting of a polynucleotide sequence as shown in Tables 1-8.

13. The nucleic acid molecule of claim 12, which is labeled.

14. The nucleic acid of claim 13, wherein the label is a fluorescent label

15. An expression vector comprising the nucleic acid of claim 12.

16. A host cell comprising the expression vector of claim 15.

17. An isolated polypeptide which is encoded by a nucleic acid molecule having polynucleotide sequence as shown in Tables 1-8

18. An antibody that specifically binds a polypeptide of claim 17.

19. The antibody of claim 18, further conjugated or fused to an effector component.

20. The antibody of claim 19, wherein the effector component is a fluorescent label.

21. The antibody of claim 19, wherein the effector component is a radioisotope.

22. The antibody of claim 19, which is an antibody fragment.

23. The antibody of claim 19, which is a humanized antibody

24. A method of detecting a cell undergoing angiogenesis in a biological sample from a patient, the method comprising contacting the biological sample with an antibody of claim 18.

25. The method of claim 24, wherein the antibody is further conjugated or fused to an effector component.

26. The method of claim 25, wherein the effector component is a fluorescent label.

27. The method of detecting antibodies specific to angiogenesis in a patient, the method comprising contacting a biological sample from the patient with a polypeptide which is encoded by a nucleotide sequence of Tables 1-8.
